SEQUENCE LISTING (47 sequences)

SEQ ID NO: 1 - protein, 120 aa, synthetic construct [sequence omitted]

SEQ ID NO: 2 - protein, 107 aa, synthetic construct [sequence omitted]

SEQ ID NO: 3 - protein, 119 aa, synthetic construct [sequence omitted]

SEQ ID NO: 4 - protein, 107 aa, synthetic construct [sequence omitted]

SEQ ID NO: 5 - protein, 7 aa, synthetic construct
  source 1..7: note = Trastuzumab Chothia CDR H1
GFNIKDT

SEQ ID NO: 6 - protein, 6 aa, synthetic construct
  source 1..6: note = Trastuzumab Chothia CDR H2
YPTNGY

SEQ ID NO: 7 - protein, 11 aa, synthetic construct
  source 1..11: note = Trastuzumab Chothia/kabat/AbM CDR H3
WGGDGFYAMDY

SEQ ID NO: 8 - protein, 11 aa, synthetic construct
  source 1..11: note = Trastuzumab Chothia/Kabat/AbM CDR L1
RASQDVNTAVA

SEQ ID NO: 9 - protein, 7 aa, synthetic construct
  source 1..7: note = Trastuzumab Chothia/Kabat/AbM CDR L2
SASFLYS

SEQ ID NO: 10 - protein, 9 aa, synthetic construct
  source 1..9: note = Trastuzumab Chothia/Kabat/IMGT/AbM CDR L3
QQHYTTPPT

SEQ ID NO: 11 - protein, 5 aa, synthetic construct
  source 1..5: note = Trastuzumab Kabat CDR H1
DTYIH

SEQ ID NO: 12 - protein, 17 aa, synthetic construct
  source 1..17: note = Trastuzumab Kabat CDR H2
RIYPTNGYTRYADSVKG

SEQ ID NO: 13 - protein, 8 aa, synthetic construct
  source 1..8: note = Trastuzumab IMGT CDR H1
GFNIKDTY

SEQ ID NO: 14 - protein, 8 aa, synthetic construct
  source 1..8: note = Trastuzumab IMGT CDR H2
IYPTNGYT

SEQ ID NO: 15 - protein, 13 aa, synthetic construct
  source 1..13: note = Trastuzumab IMGT CDR H3
SRWGGDGFYAMDY

SEQ ID NO: 16 - protein, 6 aa, synthetic construct
  source 1..6: note = Trastuzumab IMGT CDR L1
QDVNTA

SEQ ID NO: 17 - skipped

SEQ ID NO: 18 - protein, 10 aa, synthetic construct
  source 1..10: note = Trastuzumab AbM CDR H1
GFNIKDTYIH

SEQ ID NO: 19 - protein, 10 aa, synthetic construct
  source 1..10: note = Trastuzumab AbM CDR H2
RIYPTNGYTR

SEQ ID NO: 20 - protein, 7 aa, synthetic construct
  source 1..7: note = Pertuzumab Chothia CDR H1
GFTFTDY

SEQ ID NO: 21 - protein, 6 aa, synthetic construct
  source 1..6: note = Pertuzumab Chothia CDR H2
NPNSGG

SEQ ID NO: 22 - protein, 10 aa, synthetic construct
  source 1..10: note = Pertuzumab Chothia/Kabat/AbM CDR H3
NLGPSFYFDY

SEQ ID NO: 23 - protein, 11 aa, synthetic construct
  source 1..11: note = Pertuzumab Chothia/Kabat/AbM CDR L1
KASQDVSIGVA

SEQ ID NO: 24 - protein, 7 aa, synthetic construct
  source 1..7: note = Pertuzumab Chothia/Kabat/AbM CDR L2
SASYRYT

SEQ ID NO: 25 - protein, 9 aa, synthetic construct
  source 1..9: note = Pertuzumab Chothia/Kabat/IMGT/AbM CDR L3
QQYYIYPYT

SEQ ID NO: 26 - protein, 5 aa, synthetic construct
  source 1..5: note = Pertuzumab Kabat CDR H1
DYTMD

SEQ ID NO: 27 - protein, 17 aa, synthetic construct
  source 1..17: note = Pertuzumab Kabat CDR H2
DVNPNSGGSIYNQRFKG

SEQ ID NO: 28 - protein, 8 aa, synthetic construct
  source 1..8: note = Pertuzumab IMGT CDR H1
GFTFTDYT

SEQ ID NO: 29 - protein, 8 aa, synthetic construct
  source 1..8: note = Pertuzumab IMGT CDR H2
VNPNSGGS

SEQ ID NO: 30 - protein, 12 aa, synthetic construct
  source 1..12: note = Pertuzumab IMGT CDR H3
ARNLGPSFYFDY

SEQ ID NO: 31 - protein, 6 aa, synthetic construct
  source 1..6: note = Pertuzumab IMGT CDR L1
QDVSIG

SEQ ID NO: 32 - skipped

SEQ ID NO: 33 - protein, 10 aa, synthetic construct
  source 1..10: note = Pertuzumab AbM CDR H1
GFTFTDYTMD

SEQ ID NO: 34 - protein, 10 aa, synthetic construct
  source 1..10: note = Pertuzumab AbM CDR H2
DVNPNSGGSI

SEQ ID NO: 35 - protein, 330 aa, synthetic construct
  source 1..330: note = CH of Trastuzumab/Pertuzumab
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 107 aa, synthetic construct [sequence omitted]

SEQ ID NO: 37 - protein, 450 aa, synthetic construct
  source 1..450: note = heavy chain of Trastuzumab
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
  source 1..214: note = light chain of Trastuzumab
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 449 aa, synthetic construct
  source 1..449: note = heavy chain of Pertuzumab
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, synthetic construct
  source 1..214: note = light chain of Pertuzumab
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 4 aa, synthetic construct
  source 1..4: note = oligopeptide
GGFG

SEQ ID NO: 42 - protein, 4 aa, synthetic construct
  source 1..4: note = oligopeptide
GGVA

SEQ ID NO: 43 - protein, 4 aa, synthetic construct
  source 1..4: note = oligopeptide
GFLG

SEQ ID NO: 44 - protein, 4 aa, synthetic construct
  source 1..4: note = oligopeptide
EAAA

SEQ ID NO: 45 - protein, 5 aa, synthetic construct
  source 1..5: note = oligopeptide
GGGGG

SEQ ID NO: 46 - protein, 5 aa, synthetic construct
  source 1..5: note = Linker subunit
GGGGS

SEQ ID NO: 47 - protein, 20 aa, synthetic construct
  source 1..20: note = (GGGGS)4
GGGGSGGGGSGGGGSGGGGS

(19) United States
(12) Patent Application Publication
(10) Pub. No.: US 2026/0241022 A1
(43) Pub. Date: Aug. 20, 2026

(54) Conjugate having targeting effect, and preparation method therefor and use thereof

(71) Applicant: SICHUAN KELUN-BIOTECH BIOPHARMACEUTICAL CO., LTD., Chengdu, Sichuan (CN)

(72) Inventors: Qiang Tian, Chengdu, Sichuan (CN); Yitao Zhang, Chengdu, Sichuan (CN); Jian Ye, Chengdu, Sichuan (CN); Dong Long, Chengdu, Sichuan (CN); Qin Zhou, Chengdu, Sichuan (CN); Hu Long, Chengdu, Sichuan (CN); Yu Yang, Chengdu, Sichuan (CN); Xiaoxi Yuan, Chengdu, Sichuan (CN); Hongmei Song, Chengdu, Sichuan (CN); Junyou Ge, Chengdu, Sichuan (CN)

(73) Assignee: SICHUAN KELUN-BIOTECH BIOPHARMACEUTICAL CO., LTD., Chengdu, Sichuan (CN)

(21) Appl. No.: 19/469,644

(22) PCT Filed: May 10, 2024
(86) PCT No.: PCT/CN2024/092439

§ 371 Date: Sep. 26, 2025

(30) Foreign Application Priority Data
May 12, 2023 (CN) 202310538792.7
Sep. 27, 2023 (CN) 202311265152.X
Feb. 6, 2024 (CN) 202410173714.6

(51) Int. Cl. A61K 47/68; A61P 35/00; C07K 16/32

(52) U.S. Cl. CPC A61K 47/68037; A61K 47/6855; A61K 47/6889; A61P 35/00; C07K 16/32

(57) ABSTRACT

A conjugate having a targeting effect, and a preparation method therefor and a use thereof. The conjugate has a structure shown in formula Ab-[M-L-E-D]ₓ. A drug is selected from a DNA topoisomerase inhibitor. An antibody drug conjugate prepared has a better drug-to-antibody ratio, and has an excellent targeted killing effect on solid tumors such as gastric cancer, breast cancer, lung cancer and urothelial carcinoma.

The present application is a National Phase application filed under 35 USC § 371 of International Application No. PCT/CN2024/092439, filed May 10, 2024, which is based on and claims priority to the Chinese application with application number CN 202310538792.7 (filed on May 12, 2023), the Chinese application with application number CN 202311265152.X (filed on Sep. 27, 2023), and the Chinese application with application number CN 202410173714.6 (filed on Feb. 6, 2024). All the contents of these applications are hereby incorporated into the present application in their entirety.

### TECHNICAL FIELD

The present application relates to the field of targeted therapy, and specifically to a conjugate having targeting effect, preparation method therefor and use thereof.

### BACKGROUND ART

Tumor treating antibody-drug conjugate (ADC) is typically composed of a monoclonal antibody, a bioactive molecule (primarily a tumor-killing cytotoxin) and a linker. The bioactive molecule is covalently coupled to the antibody via the linker. The antibody recognizes a specific target on the surface of tumor cells, directing the ADC to the tumor microenvironment and cancer cell surfaces. Through endocytosis, the ADC enters the cancer cells, where the bioactive molecule is released. By inhibiting the tubulin of the cancer cells or damaging the DNA of the cancer cells, the bioactive molecule kills the cancer cells while minimizing damage to normal tissue cells.

The ErbB family of receptor tyrosine kinases are important mediators of cell growth, differentiation, and survival. This family includes four members: epidermal growth factor receptor (EGFR or ErbB1), Her2 (ErbB2), Her3 (ErbB3) and Her4 (ErbB4). In clinical practice, the anti-ErbB2 antibody Trastuzumab (trade name Herceptin) is often used to treat breast cancer with high expression of ErbB2, but the clinical response rate remains low. In order to improve the therapeutic efficacy, in recent years, conjugates (e.g., Trastuzumab emtansine, Disitamab vedotin, Trastuzumab deruxtecan) combining anti-ErbB2 antibodies with tubulin inhibitors such as maytansines (e.g., DM1), auristatins (e.g., MMAE) or DNA topoisomerase I inhibitors (e.g., Dxd) have been used for clinical treatment.

With the widespread clinical application of the aforementioned ADC drugs in the treatment of Her2-expressing tumors, safety issues including neurotoxicity, hematotoxicity, hepatotoxicity, and interstitial pneumonia and drug resistance issues have gradually emerged (Pharmacology & Therapeutics 2019, 200, 110-125; Breast Cancer Research and Treatment 2020, 183, 23-39; JAMA Oncol. 2021, 7, 1873-1881; Drug Deliv. 2022, 29, 1335-1344; Cancers 2023, 15, 1130; Cancers 2023, 15, 1278).

Specifically, both Disitamab vedotin and Trastuzumab deruxtecan utilize the maleimide joint MC in their linker moieties. This joint has been reported to be susceptible to reversible Michael addition and thiol exchange reactions under physiological conditions, resulting in reduced efficacy and increased toxicity (Nat Biotechnol. 2012, 32, 184-189; Bioconjugate Chem. 2015, 26, 145-152). In terms of the hydrophilicity of the linker, Disitamab vedotin and Trastuzumab deruxtecan adopt the more hydrophobic valine-citrulline (Val-Cit) and glycine-glycine-phenylalanine-glycine (Gly-Gly-Phe-Gly) linker, respectively. Literature reports that the hydrophilicity of linkers significantly affects the hydrophilicity of ADCs, thereby affecting the aggregation, pharmacokinetic properties and toxicity of ADCs (Chemical Linkers in Antibody-Drug Conjugates (ADCs), Drug Discovery Series No. 81, Chapter 3). As for the bioactive molecule moieties that play a role in killing cancer cells, Trastuzumab emtansine incorporate the microtubule inhibitor DM1 as a cytotoxin, which leads to a weak bystander effect after being matched with a non-cleavable linker; Disitamab vedotin employs the auristatin toxin MMAE as its bioactive molecule, which may associates with problems such as cumulative neurotoxicity upon repeated administration; and both of the aforementioned ADC drugs utilize non-site-specific random conjugation, resulting relatively poor homogeneity.

### CONTENTS OF THE PRESENT INVENTION

The present invention aims to improve the above problems in the drug technology in the art, specifically to provide a class of anti-Her2 antibody-drug conjugates that can be used for the treatment of Her2-expressing tumors. The antibody-drug conjugates have good stability, uniformity, hydrophilicity, efficacy and safety.

The present application relates to an antibody-drug conjugate, and exemplarily discloses an antibody-drug conjugate with a structure represented by the general formula Ab-[M-L-E-D]ₓ with Trastuzumab as a targeting part. The results show that the conjugate has a relatively good drug-to-antibody conjugation ratio, and the conjugate has good binding activity and proliferation inhibition on Her2-positive cells, and has an excellent targeted killing effect on Her2-positive tumors (e.g., breast cancer, lung cancer or gastric cancer). Therefore, the present application provides an antibody-drug conjugate for treating Her2-expressing cancer, a pharmaceutical composition containing the antibody-drug conjugate, and their use in treating Her2-expressing cancer.

### Antibody-Drug Conjugate

In one aspect, the present application provides an antibody-drug conjugate having a structure represented by the formula Ab-[M-L-E-D]ₓ, wherein:

Ab represents an antibody or antigen-binding fragment thereof capable of specifically binding to epidermal growth factor receptor 2 (Her2), a member of the ErbB family of receptor tyrosine kinases:
M represents a joint connecting the antibody or antigen-binding fragment thereof;
L represents a structural fragment connecting M and E;
E represents a structural fragment connecting L and D;
D represents a cytotoxic drug fragment;
x is 1 to 10.

In the antibody-drug conjugate, the cytotoxic drug can be connected to the antibody or antigen-binding fragment thereof through the “M-L-E” fragment as shown in the present application.

In some embodiments, M is selected from the group consisting of the following substituted or unsubstituted structural fragments:

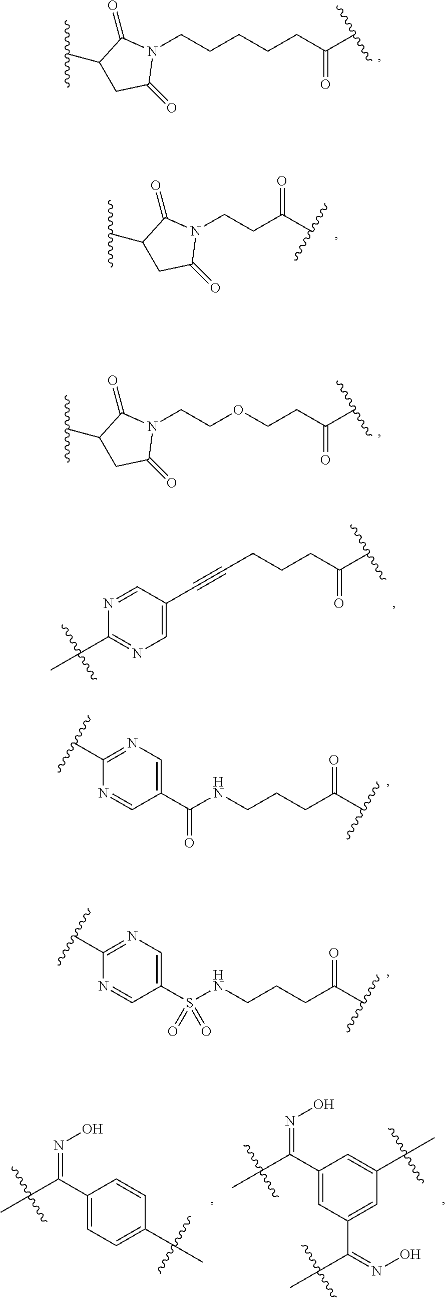

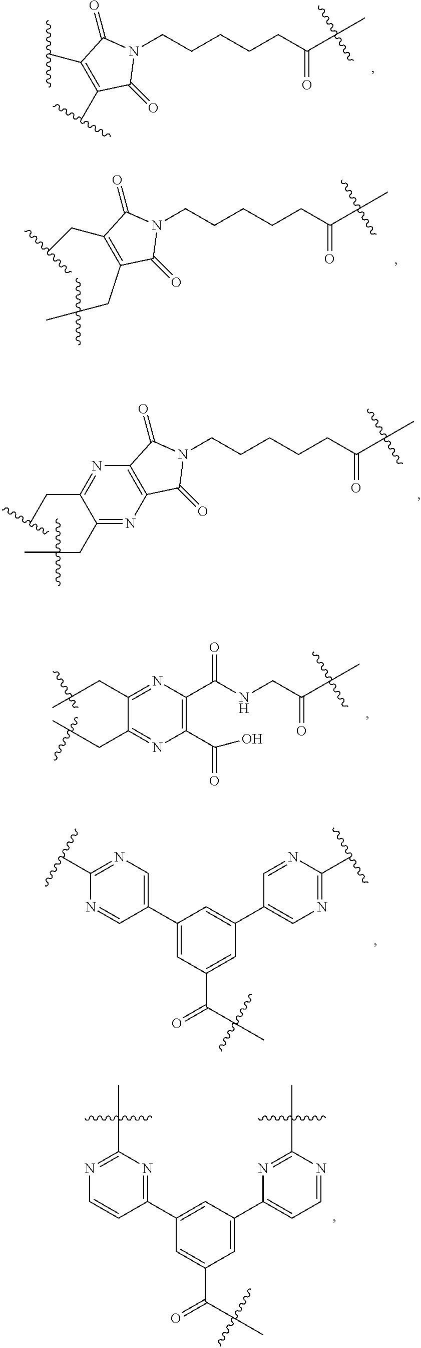 [SMILES: *C(=O)CCCCCN1C(=O)C(*)=C(*)C1=O ; *CC1=C(C*)C(=O)N(CCCCCC(*)=O)C1=O ; *Cc1nc2c(nc1C*)C(=O)N(CCCCCC(*)=O)C2=O ; *Cc1nc(C(=O)O)c(C(=O)NCC(*)=O)nc1C* ; *C(=O)c1cc(-c2cnc(*)nc2)cc(-c2cnc(*)nc2)c1 ; *C(=O)c1cc(-c2ccnc(*)n2)cc(-c2ccnc(*)n2)c1]

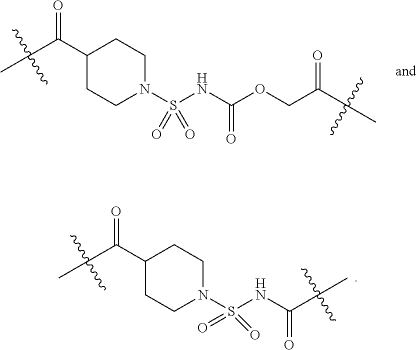 [SMILES: *C(=O)COC(=O)NS(=O)(=O)N1CCC(C(*)=O)CC1 ; *C(=O)NS(=O)(=O)N1CCC(C(*)=O)CC1]

In some embodiments, M is selected from the group consisting of the following substituted or unsubstituted structural fragments:

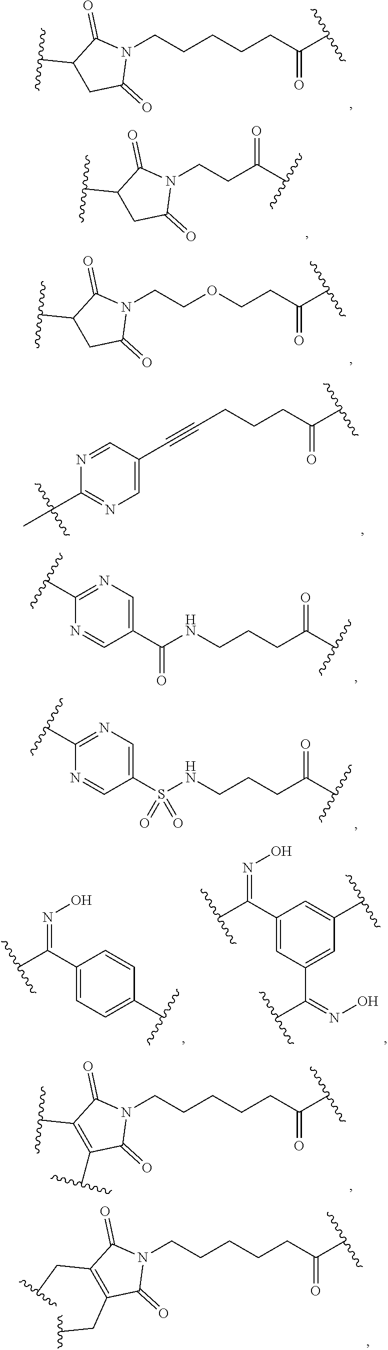

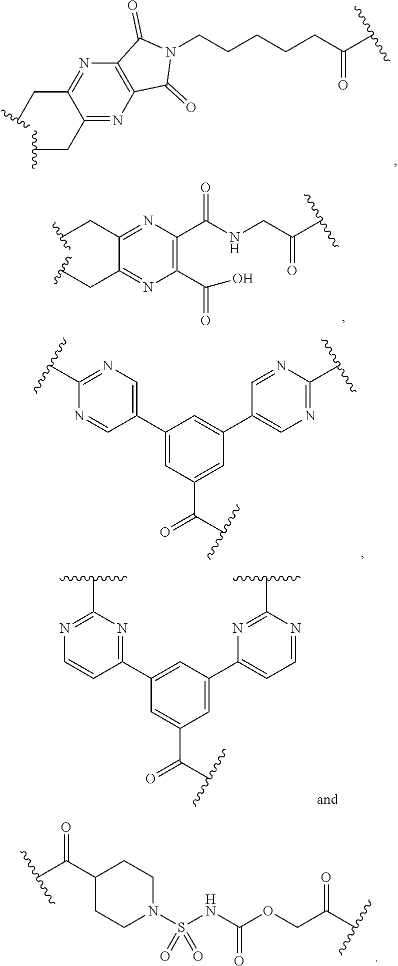 [SMILES: *Cc1nc2c(nc1C*)C(=O)N(CCCCCC(*)=O)C2=O ; *Cc1nc(C(=O)O)c(C(=O)NCC(*)=O)nc1C* ; *C(=O)c1cc(-c2cnc(*)nc2)cc(-c2cnc(*)nc2)c1 ; *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the group consisting of the following substituted or unsubstituted structural fragments:

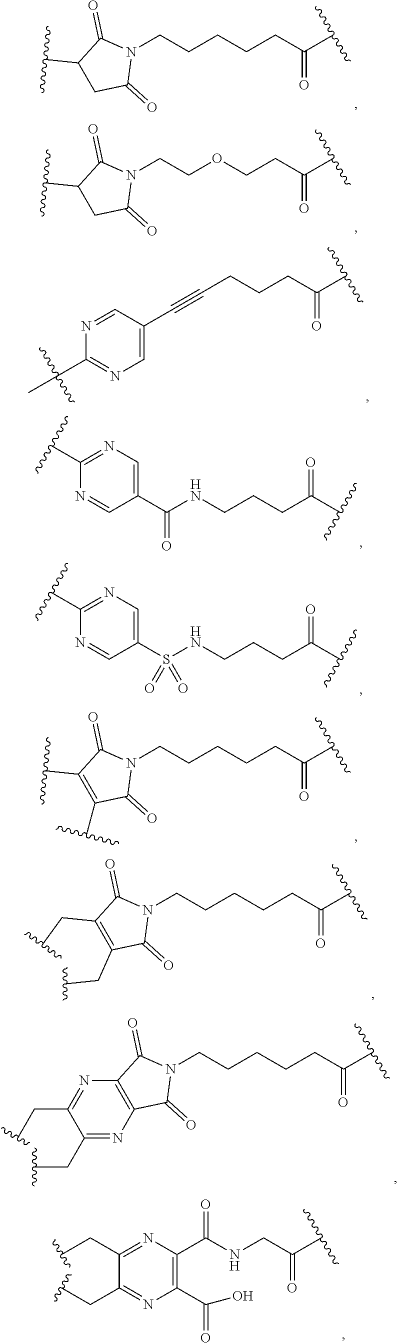

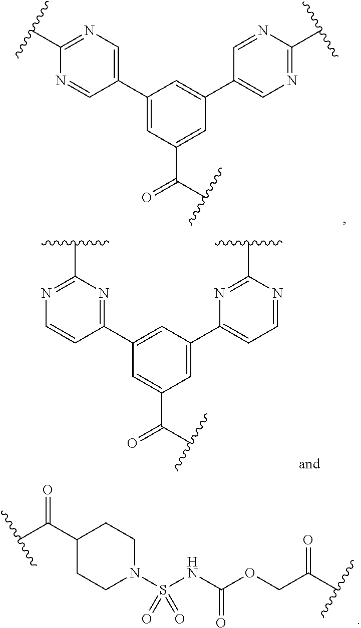 [SMILES: *C(=O)c1cc(-c2cnc(*)nc2)cc(-c2cnc(*)nc2)c1 ; *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the group consisting of the following substituted or unsubstituted structural fragments:

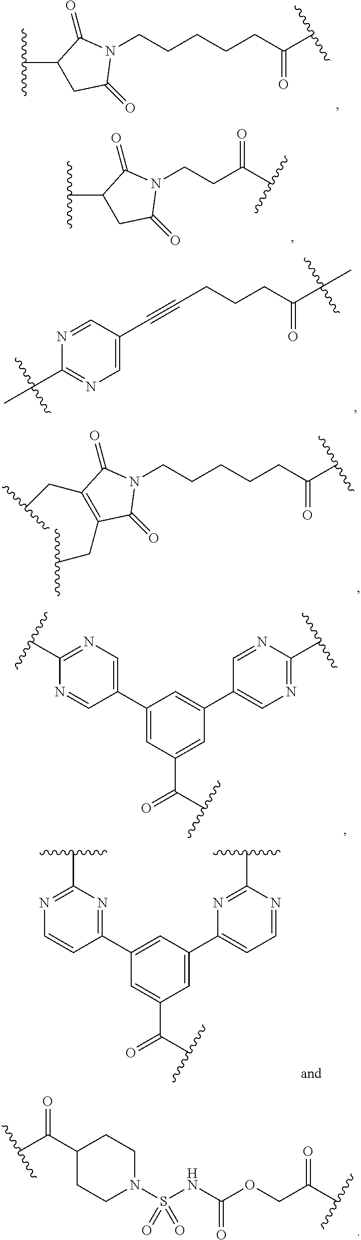

In some embodiments, M is selected from the group consisting of the following substituted or unsubstituted structural fragments:

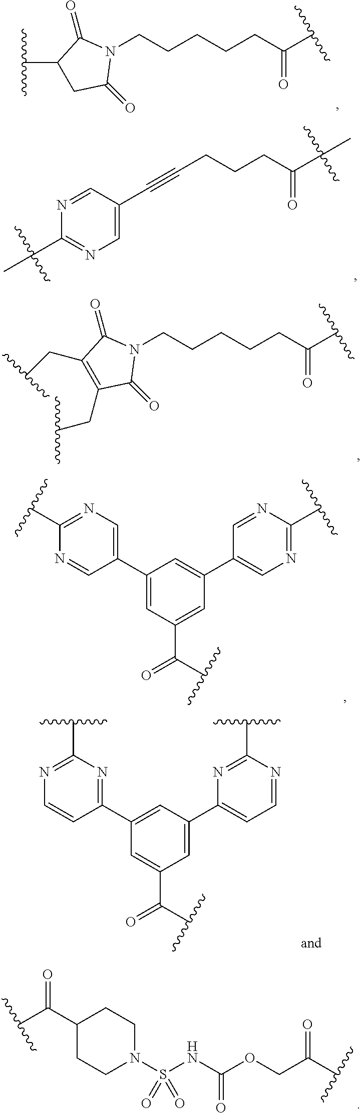 [SMILES: *C(=O)CCCCCN1C(=O)CC(*)C1=O ; *C(=O)CCCC#Cc1cnc(*)nc1 ; *CC1=C(C*)C(=O)N(CCCCCC(*)=O)C1=O ; *C(=O)c1cc(-c2cnc(*)nc2)cc(-c2cnc(*)nc2)c1 ; *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the group consisting of the following substituted or unsubstituted structural fragments:

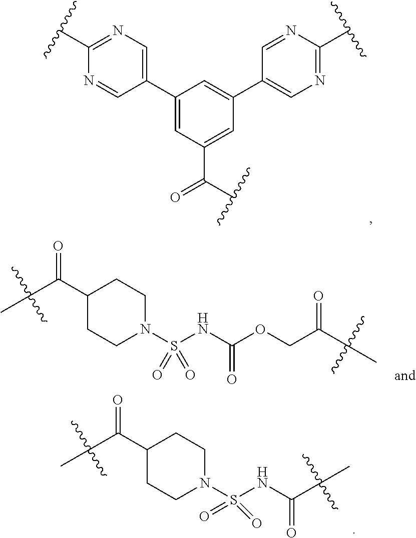

In some embodiments, M is selected from the group consisting of the following substituted or unsubstituted structural fragments:

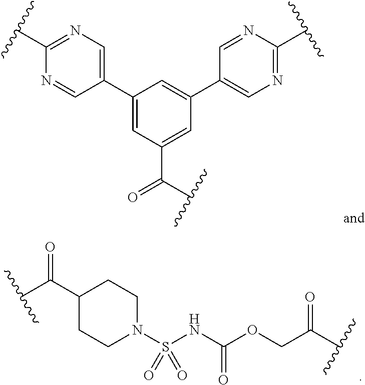 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1]

In some embodiments, M is selected from the group consisting of the following substituted or unsubstituted structural fragments:

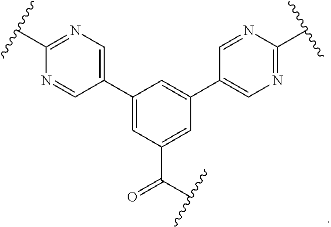 [SMILES: *C(=O)c1cc(-c2cnc(*)nc2)cc(-c2cnc(*)nc2)c1]

In some embodiments, M is selected from the group consisting of the following substituted or unsubstituted structural fragments:

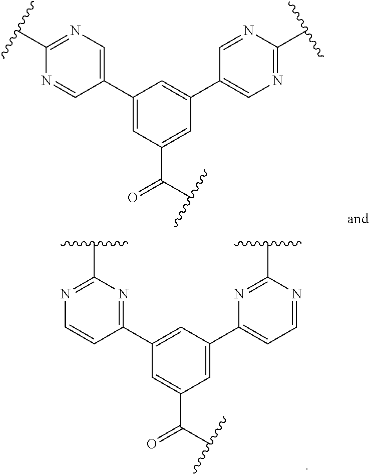 [SMILES: *C(=O)c1cc(-c2cnc(*)nc2)cc(-c2cnc(*)nc2)c1 ; *C(=O)c1cc(-c2ccnc(*)n2)cc(-c2ccnc(*)n2)c1]

In some embodiments, M is selected from the group consisting of the following substituted or unsubstituted structural fragments:

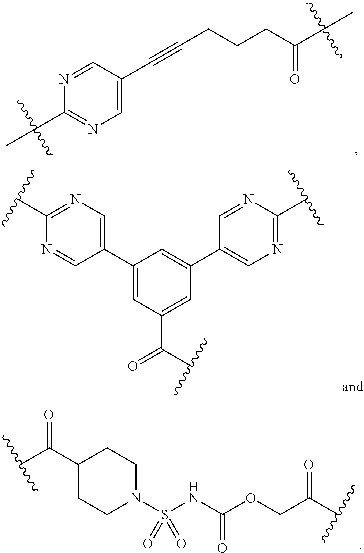 [SMILES: *C(=O)CCCC#Cc1cnc(*)nc1 ; *C(=O)c1cc(-c2cnc(*)nc2)cc(-c2cnc(*)nc2)c1 ; *C(=O)COC(=O)NS(=O)(=O)N1CCC(C(*)=O)CC1]

In some embodiments, L is selected from the group consisting of the substituted or unsubstituted structural fragments composed of one or more of the following groups: C₁₋₆ alkylene, 6- to 10-membered aryl, 5- to 6-membered heteroaryl, 9- to 12-membered nitrogen-containing heterocyclyl, —N(R′)—, —NH(R′), —N(R′)₂, —NHCH(R′)—C(═O)—, carbonyl group, —O—, natural amino acids or unnatural amino acids or analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Glu(R′), Lys(R′), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly (GGFG, SEQ ID NO: 41), Gly-Gly-Val-Ala (GGVA, SEQ ID NO: 42), Gly-Phe-Leu-Gly (GFLG, SEQ ID NO: 43), Glu-Ala-Ala-Ala (EAAA, SEQ ID NO: 44), Gly-Gly-Gly-Gly-Gly (GGGGG, SEQ ID NO: 45)),

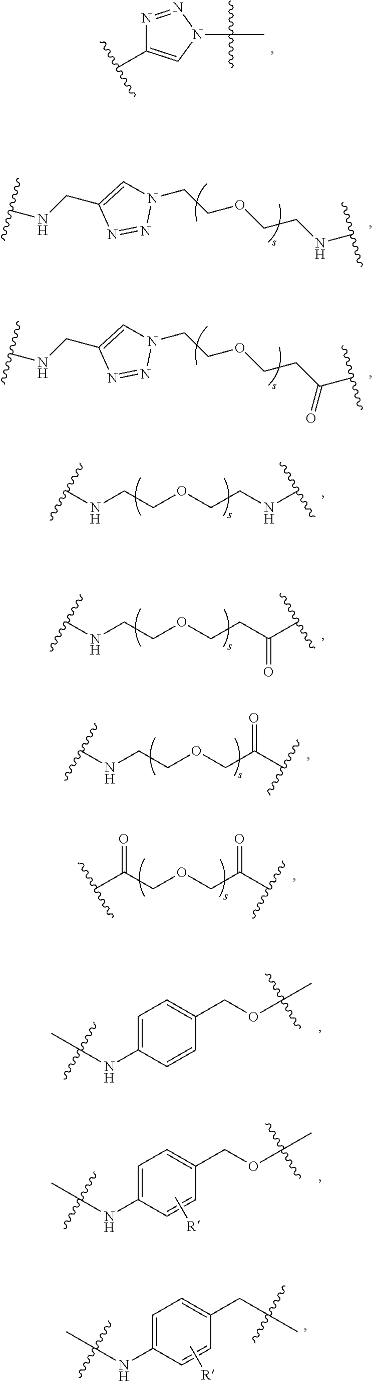

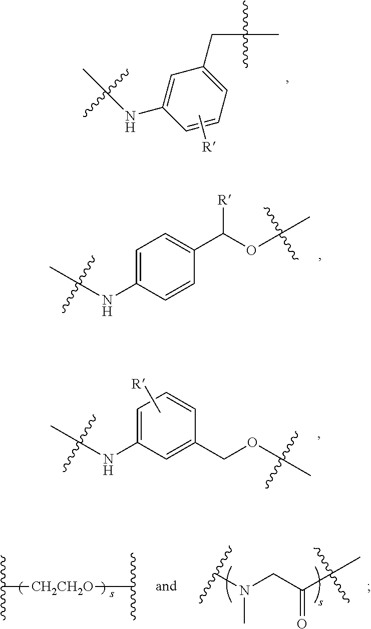

wherein R′ is composed of one or more of the following groups, including but not limited to: hydrogen, C₁₋₆ alkyl, C₁₋₆ alkylene, amino, hydroxyl, carboxyl, acyl, —O—, —C₁₋₆ alkylene-CO₂H, —C₁₋₆ alkylene-SO₃H, —SO₃H, —PO₃H₂, —C₁₋₆ alkylene-NHC₁₋₆ alkyl, —C₁₋₆ alkylene-N(C₁₋₆ alkyl)₂, —C₁₋₆ alkylene-heterocycle, —CH₂N(C₁₋₆ alkyl)-C(═O)C₁₋₆ alkylene-heterocycle, —CH₂NH—SO₃H, —CH₂N(C₁₋₆ alkyl)-SO₃H, —CH₂NHC₁₋₆ alkylene-SO₃H, —CH₂N(C₁₋₆ alkyl)C₁₋₆ alkylene-SO₃H, —CH₂N(C₁₋₆ alkylene-SO₃H)₂, —CH₂N*(C₁₋₆ alkylene-SO₃H)₃, —CH₂N⁺(C₁₋₆ alkyl)₂-C₁₋₆ alkylene-SO₃H, —CH₂N(C₁₋₆ alkyl)-C(═O)C₁₋₆ alkylene-N⁺(C₁₋₆ alkylene-SO₃H)₃, —CH₂NH—C(═O)C₁₋₆ alkylene-N⁺(C₁₋₆ alkylene-SO₃H)₃, —CH₂N(C₁₋₆ alkyl)-C(═O)C₁₋₆ alkylene-N⁺(C₁₋₆ alkyl)₃, —CH₂NH—C(═O)C₁₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₂-CH₂CO₂H, —CH₂N(C₁₋₆ alkyl)-C₁₋₆ alkylene-CO₂H, —CH₂N⁺(C₁₋₆ alkyl)₂-C₁₋₆ alkylene-CO₂H, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—(OCH₂CH₂)ᵣ—OC₁₋₆ alkyl, —(CH₂N(Me)-C(═O))ᵣ—C₁₋₆ alkyl, polyethylene glycol fragment containing 1 to 10 EO units (i.e. —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-a-propionyl), NOTA (1,4,7-triazacyclononane-N,N′,N″-triacetic acid residue), —C₁₋₆ alkylene-N(C₁₋₆ alkyl)-DOTA, —C₁₋₆ alkyl-N(C₁₋₆ alkyl)-DOTAGA, or —C₁₋₆ alkyl-N(C₁₋₆ alkyl)-NOTA, wherein r is an integer selected from 1 to 20; s is an integer selected from 1 to 20.

In some embodiments, r is an integer selected from 1 to 15, such as an integer of 1 to 12, 3 to 12, 1 to 10, 1 to 8, 3 to 8, 1 to 6, 1 to 4, 1 to 2, for example, r is 1, 2, 3, 4, 5, 6, 7, 8, 9, 10, 11, 12, 13, 14 or 15.

In some embodiments, s is an integer selected from 1 to 15, such as an integer of 1 to 12, 3 to 12, for example, s is 1, 2, 3, 4, 5, 6, 7, 8, 9, 10, 11, 12, 13, 14 or 15.

In some embodiments, s is denoted as n.

In some embodiments, “—NOTA” refers to

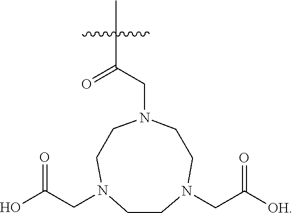 [SMILES: *C(=O)CN1CCN(CC(=O)O)CCN(CC(=O)O)CC1]

In some embodiments, “-DOTA” refers to

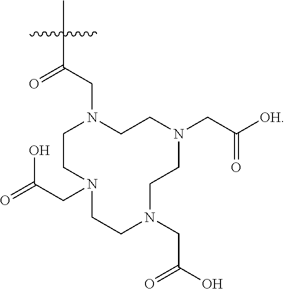 [SMILES: *C(=O)CN1CCN(CC(=O)O)CCN(CC(=O)O)CCN(CC(=O)O)CC1]

In some embodiments, “-DOTAGA” refers to

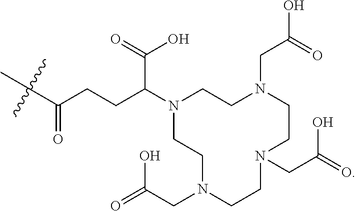 [SMILES: *C(=O)CCC(C(=O)O)N1CCN(CC(=O)O)CCN(CC(=O)O)CCN(CC(=O)O)CC1]

In some embodiments, L is selected from the group consisting of the substituted or unsubstituted structural fragments composed of one or more of the following groups: C₁₋₆ alkylene, 6- to 10-membered aryl, 5- to 6-membered heteroaryl, 9- to 12-membered nitrogen-containing heterocyclyl, —N(R′)—, —NH(R′), —N(R′)₂, carbonyl group, —O—, natural amino acids or unnatural amino acids or analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

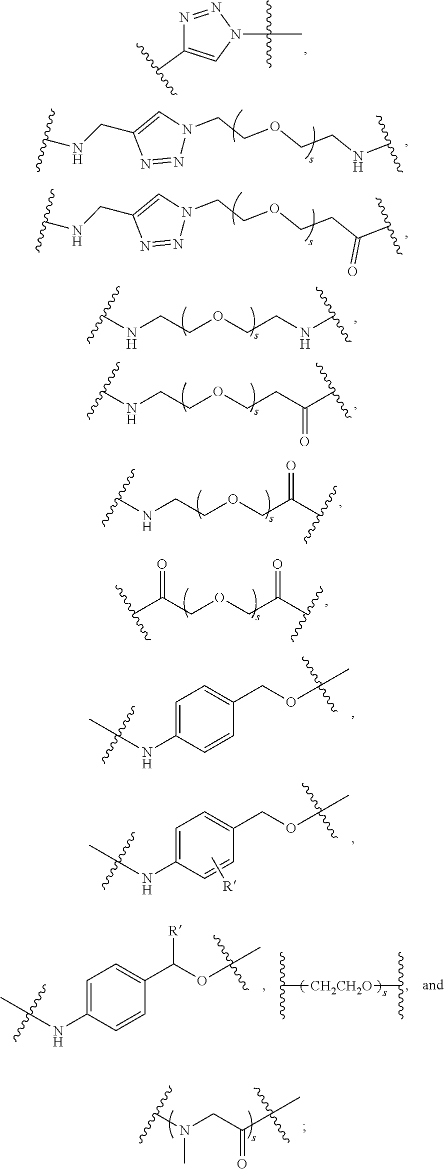

Wherein R′ represents hydrogen, C₁₋₆ alkyl, —C₁₋₆ alkylene-CO₂H, —C₁₋₆ alkylene-SO₃H, —SO₃H, —PO₃H₂, —C₁₋₆ alkylene-NHC₁₋₆ alkyl, —C₁₋₆ alkylene-N(C₁₋₆ alkyl)₂, —CH₂N(C₁₋₆ alkyl)-C(═O)C₁₋₆ alkylene-heterocycle, —CH₂NH—SO₃H, —CH₂N(C₁₋₆ alkyl)-SO₃H, —CH₂NHC₁₋₆ alkylene-SO₃H, —CH₂N(C₁₋₆ alkyl)C₁₋₆ alkylene-SO₃H, —CH₂N(C₁₋₆ alkylene-SO₃H)₂, —CH₂N⁺(C₁₋₆ alkylene-SO₃H)₃, —CH₂N⁺(C₁₋₆ alkyl)₂-C₁₋₆ alkylene-SO₃H, —CH₂N(C₁₋₆ alkyl)-C(═O)C₁₋₆ alkylene-N⁺(C₁₋₆ alkylene-SO₃H)₃, —CH₂NH—C(═O)C₁₋₆ alkylene-N⁺(C₁₋₆ alkylene-SO₃H)₃, —CH₂N(C₁₋₆ alkyl)-C(═O)C₁₋₆ alkylene-N⁺(C₁₋₆ alkyl)₃, —CH₂NH—C(═O)C₁₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₂-CH₂CO₂H, —CH₂N(C₁₋₆ alkyl)-C₁₋₆ alkylene-CO₂H, —CH₂N⁺(C₁₋₆ alkyl)₂-C₁₋₆ alkylene-CO₂H, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—(OCH₂CH₂)ᵣ—OC₁₋₆ alkyl, —(CH₂N(Me)-C(═O))ᵣ—C₁₋₆ alkyl, or polyethylene glycol fragment containing 1 to 10 EO units (i.e., —(CH₂CH₂O)ᵣ—C₁₋₆ alkyl), wherein r is an integer selected from 1 to 20; s is an integer selected from 1 to 20; in some embodiments, L is selected from the group consisting of the substituted or unsubstituted structural fragments composed of one or more of the following groups: C₁₋₆ alkylene, 6- to 10-membered aryl, 5- to 6-membered heteroaryl, —N(R′)—, carbonyl group, —O—, natural amino acids or unnatural amino acids or analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly (GGFG, SEQ ID NO: 41), Gly-Gly-Val-Ala (GGVA, SEQ ID NO: 42), Gly-Phe-Leu-Gly (GFLG, SEQ ID NO: 43), Glu-Ala-Ala-Ala (EAAA, SEQ ID NO: 44), Gly-Gly-Gly-Gly-Gly (GGGGG, SEQ ID NO: 45)),

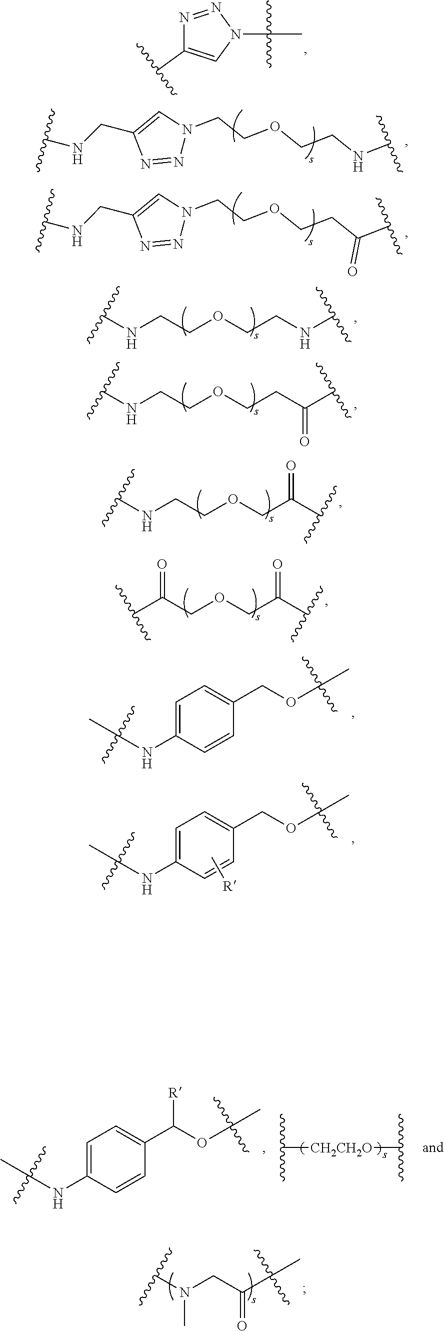

wherein R′ is composed of one or more of the following groups, including but not limited to: hydrogen, C₁₋₆ alkyl, C₁₋₆ alkylene, amino, hydroxyl, carboxyl, acyl, —O—, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, polyethylene glycol fragment containing 1 to 10 EO units (e.g., —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-a-propionyl), or NOTA (1,4,7-triazacyclononane-N,N′,N″-triacetic acid residue), wherein r is an integer selected from 1 to 20; s is an integer selected from 1 to 20.

In some embodiments, L is selected from the group consisting of the substituted or unsubstituted structural fragments composed of one or more of the following groups: C₁₋₆ alkylene, 6- to 10-membered aryl, 5- to 6-membered heteroaryl, —N(R′)—, carbonyl group, —O—, natural amino acids or unnatural amino acids or analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

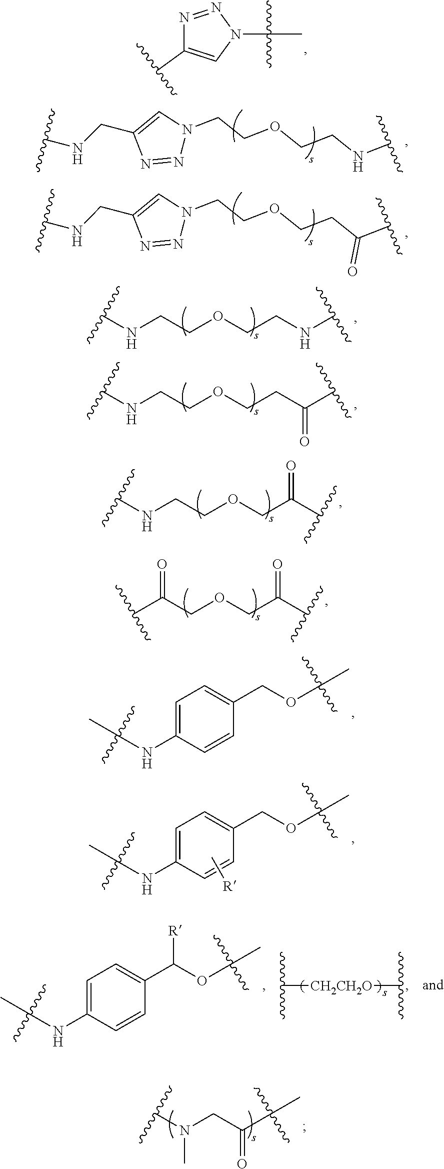

wherein R′ represents hydrogen, C₁₋₆ alkyl, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, or polyethylene glycol fragment containing 1 to 10 EO units (i.e. —(CH₂CH₂O)ᵣ—C₁₋₆ alkyl), wherein r is an integer selected from 1 to 20; s is an integer selected from 1 to 20.

In some embodiments, L is selected from the group consisting of the substituted or unsubstituted structural fragments composed of one or more of the following groups: C₁₋₆ alkylene, carbonyl group, 9- to 12-membered nitrogen-containing heterocyclyl, —NH—, Ala-Ala, Ala-Lys, Ala-Pro, Gly-Glu, Gly-Gly, Phe-Lys, Val-Ala, Val-Cit, Val-Lys, Ala-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly,

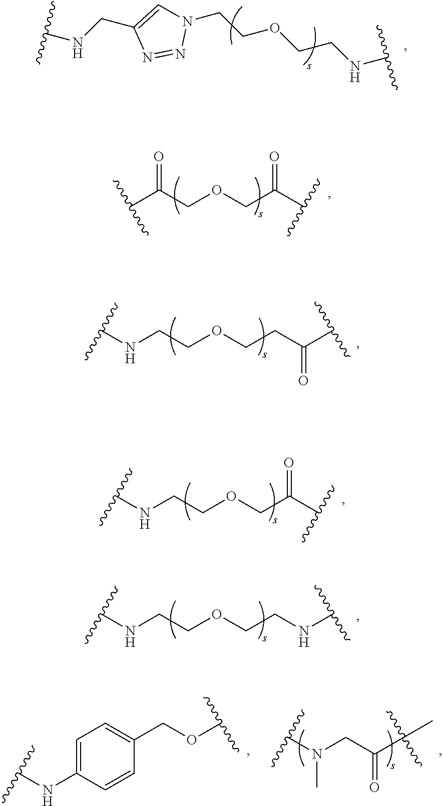

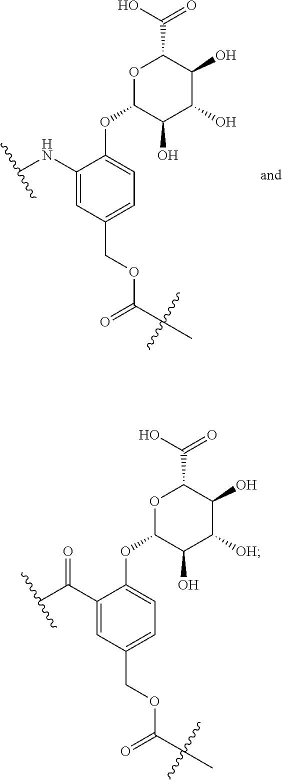

wherein s is an integer selected from 1 to 20.

In some embodiments, L is selected from the group consisting of the substituted or unsubstituted structural fragments composed of one or more of the following groups: C₁₋₆ alkylene, carbonyl group, —NH—, Ala-Ala, Ala-Lys, Ala-Pro, Gly-Glu, Gly-Gly, Phe-Lys, Val-Ala, Val-Cit, Val-Lys, Ala-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly,

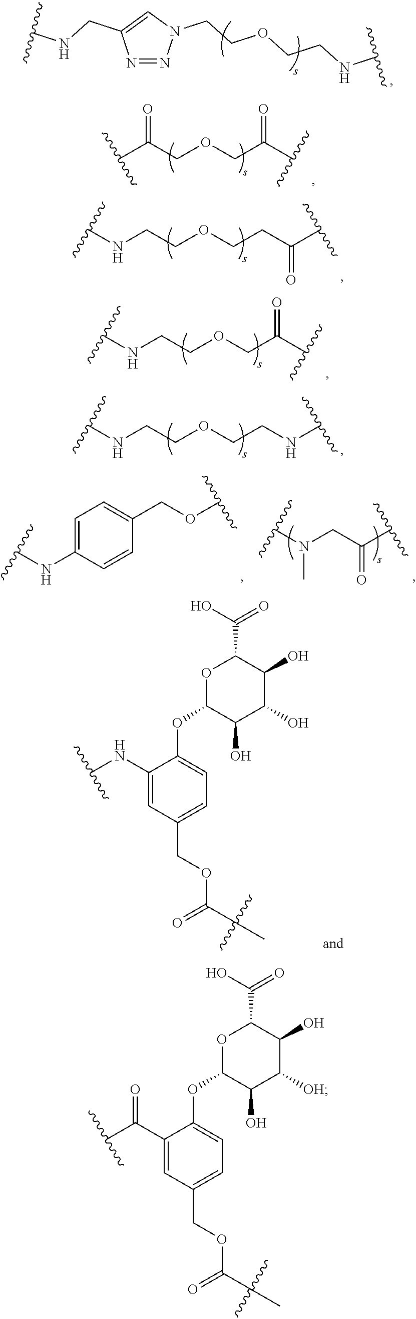

wherein s is an integer selected from 1 to 20.

In some embodiments, L is selected from the group consisting of the substituted or unsubstituted structural fragments composed of one or more of the following groups:

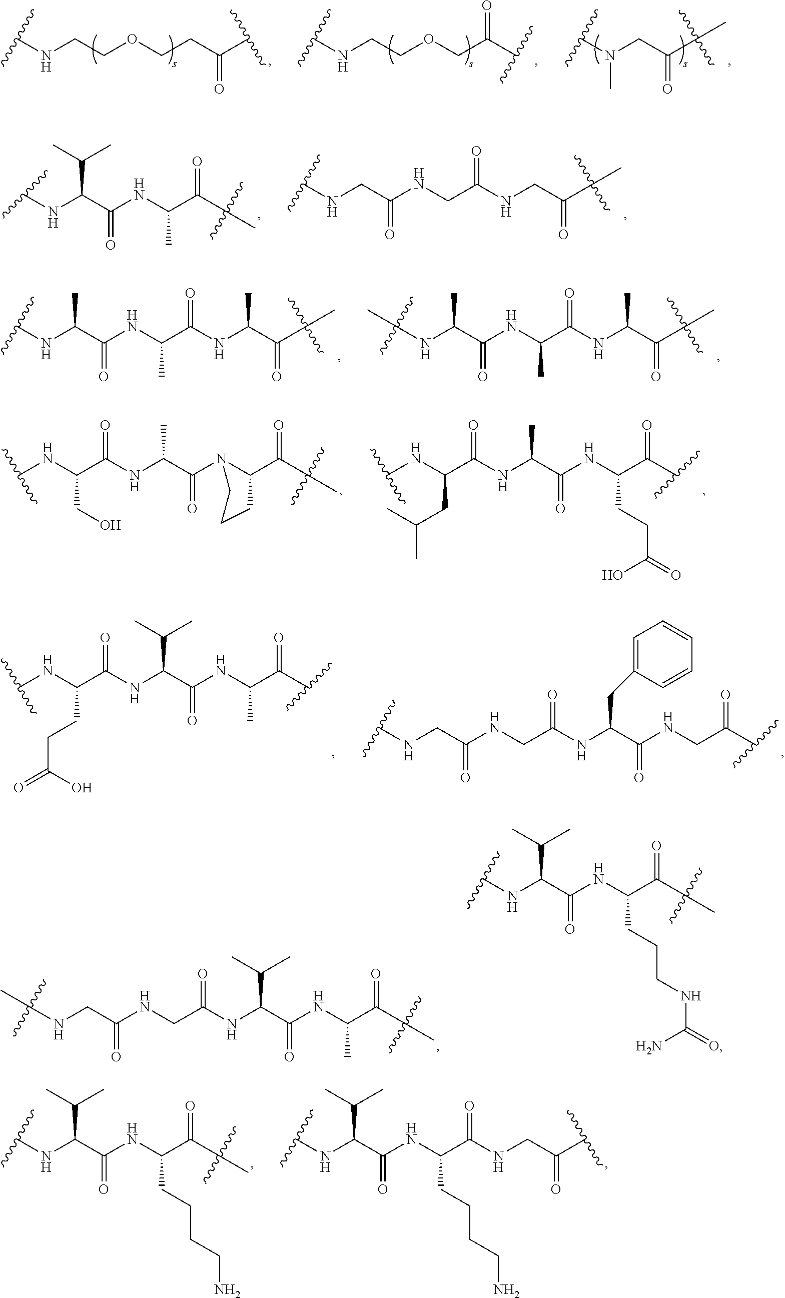

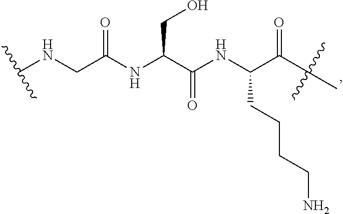

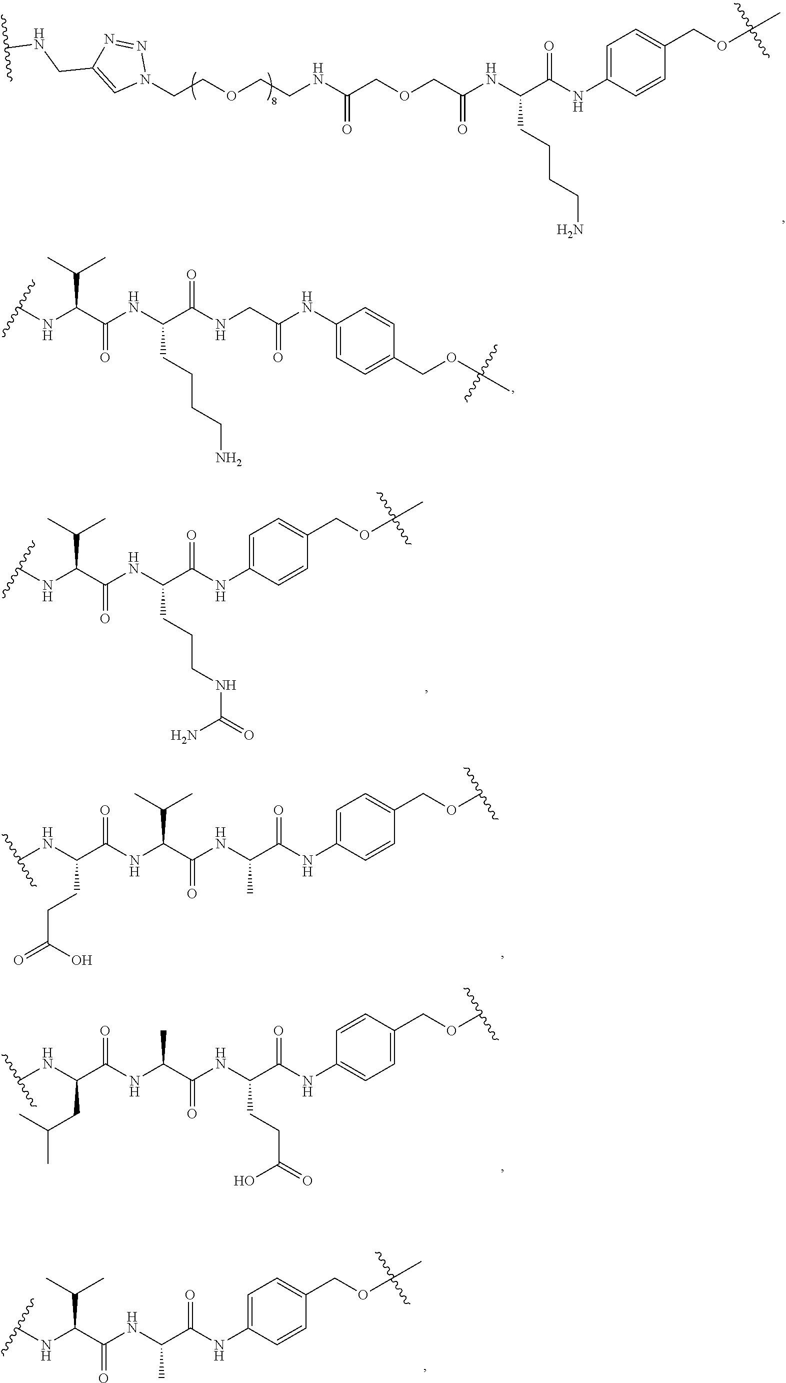

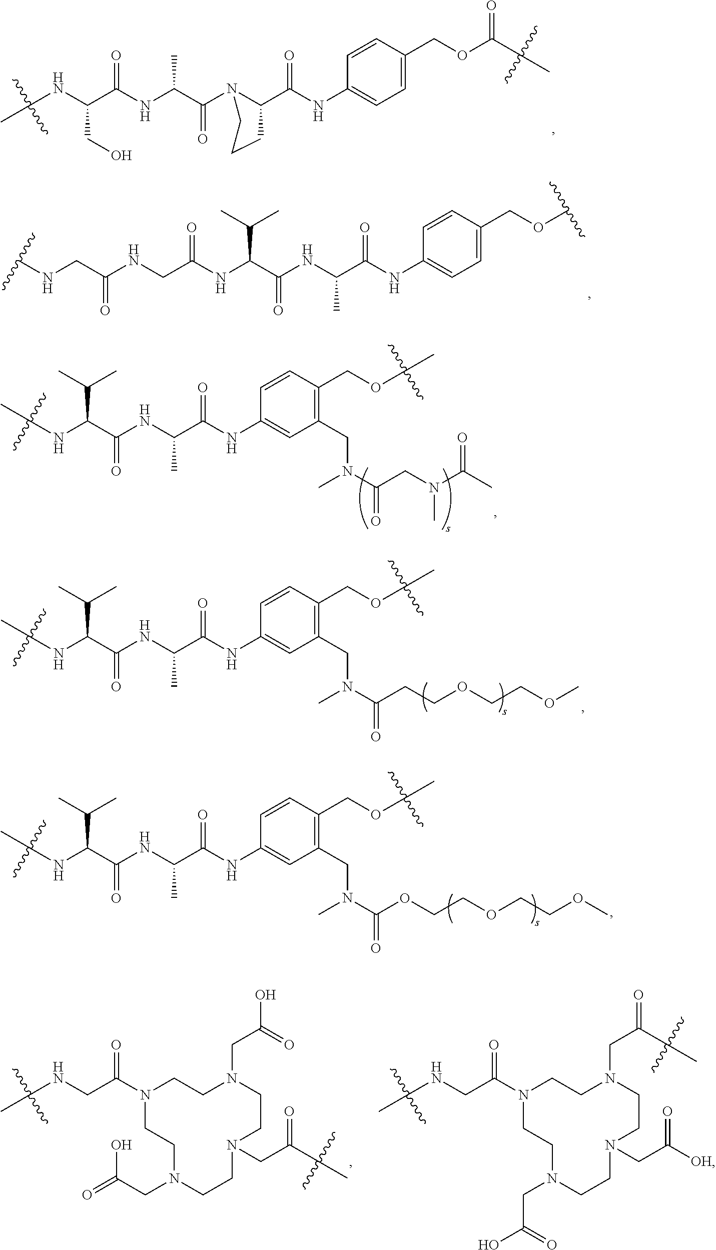

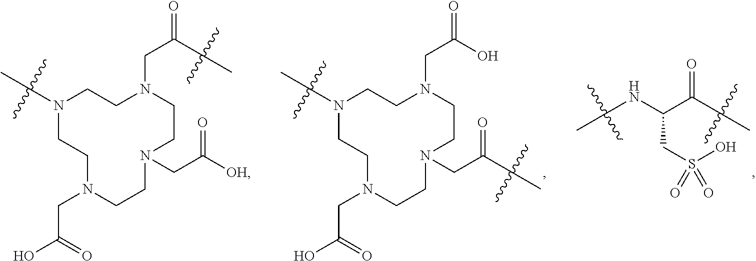

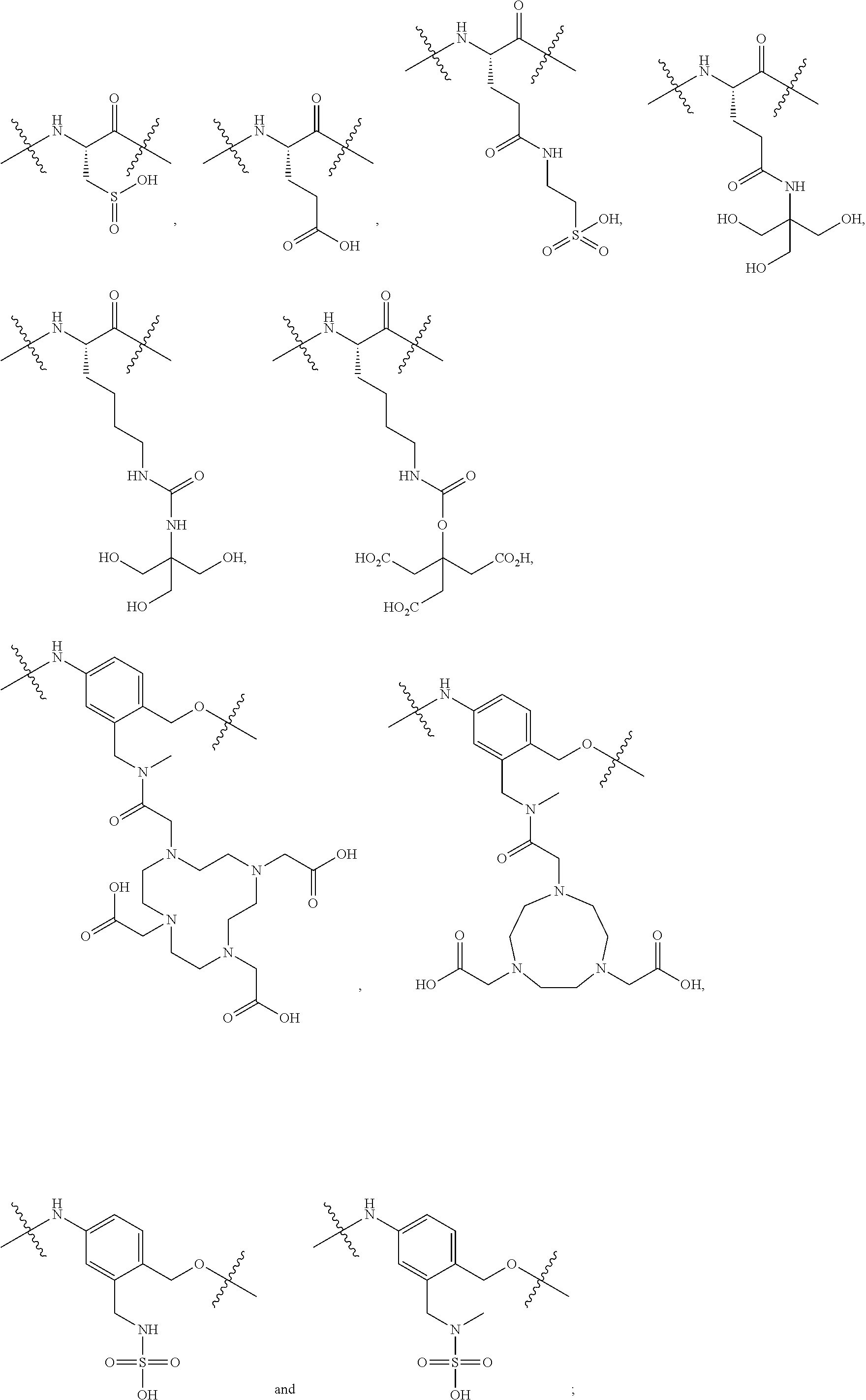

wherein s is an integer selected from 1 to 20.

In some embodiments, L is selected from the group consisting of the substituted or unsubstituted structural fragments composed of one or more of the following groups:

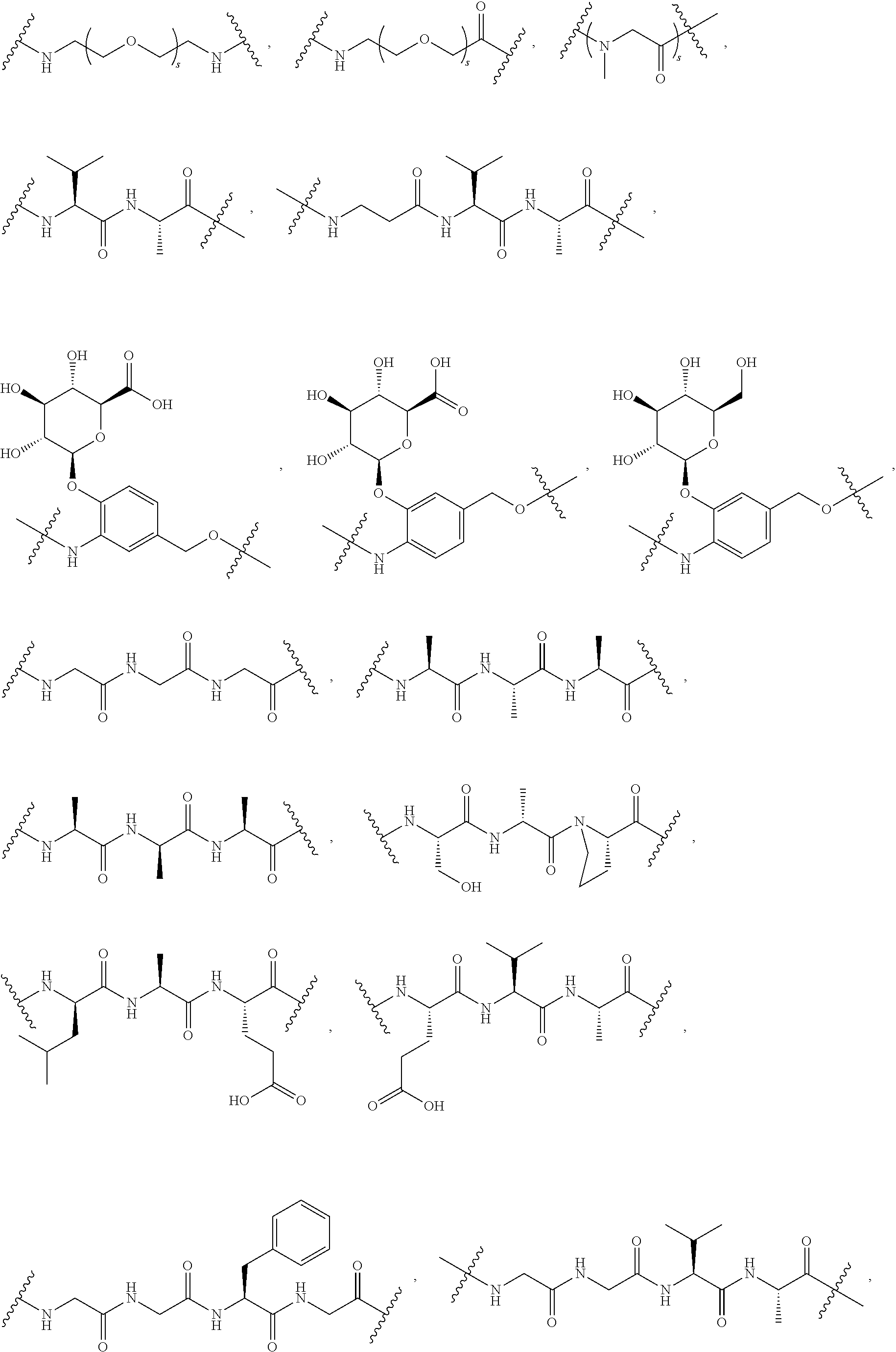

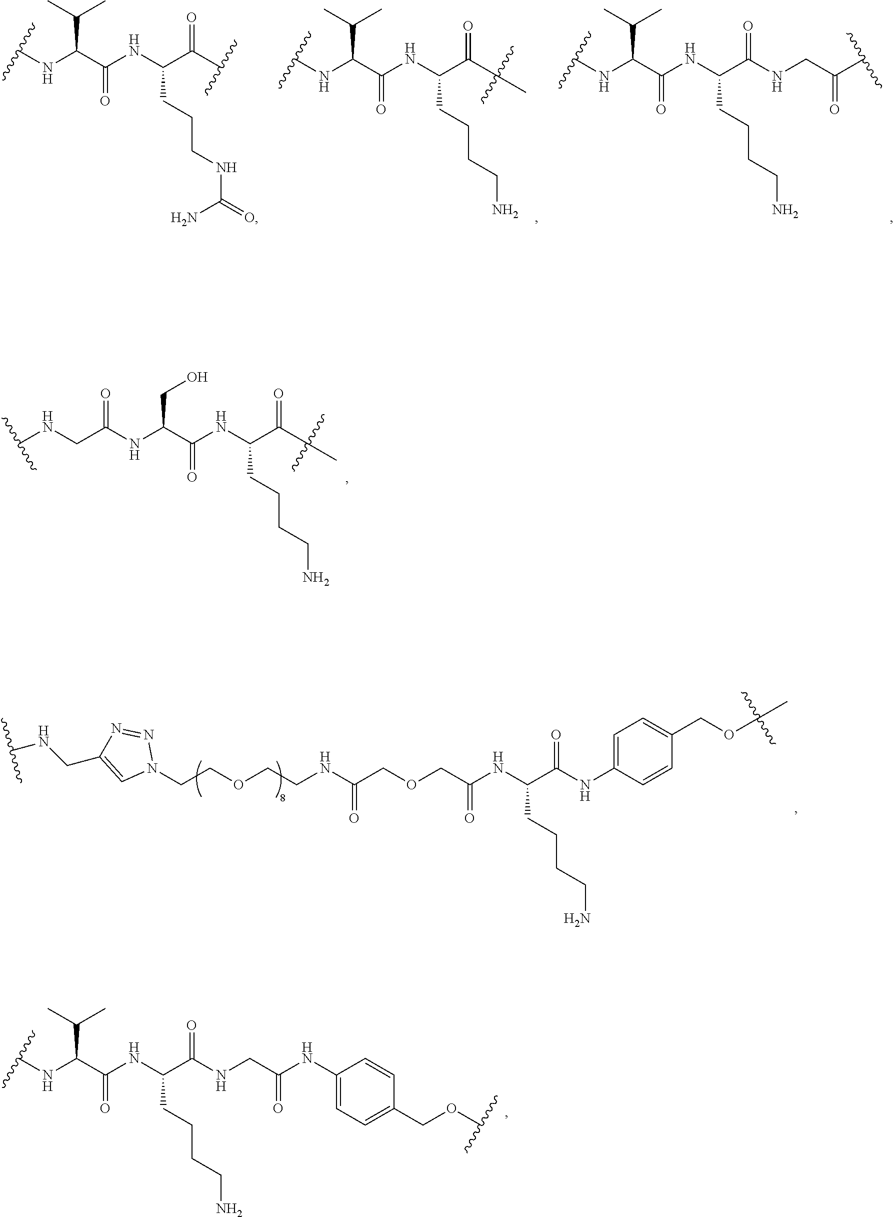

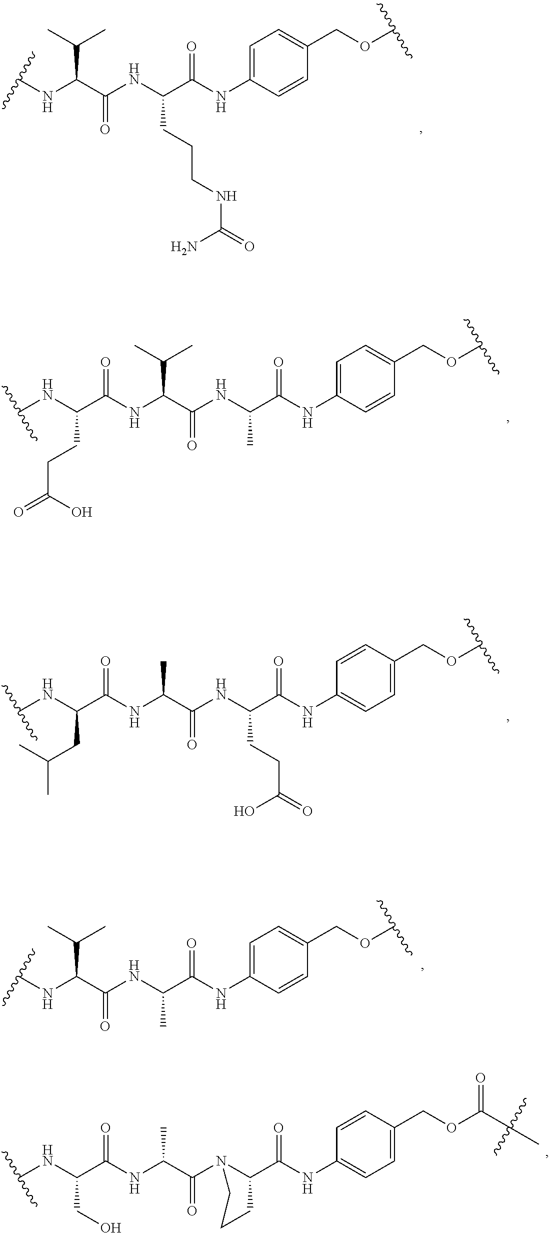

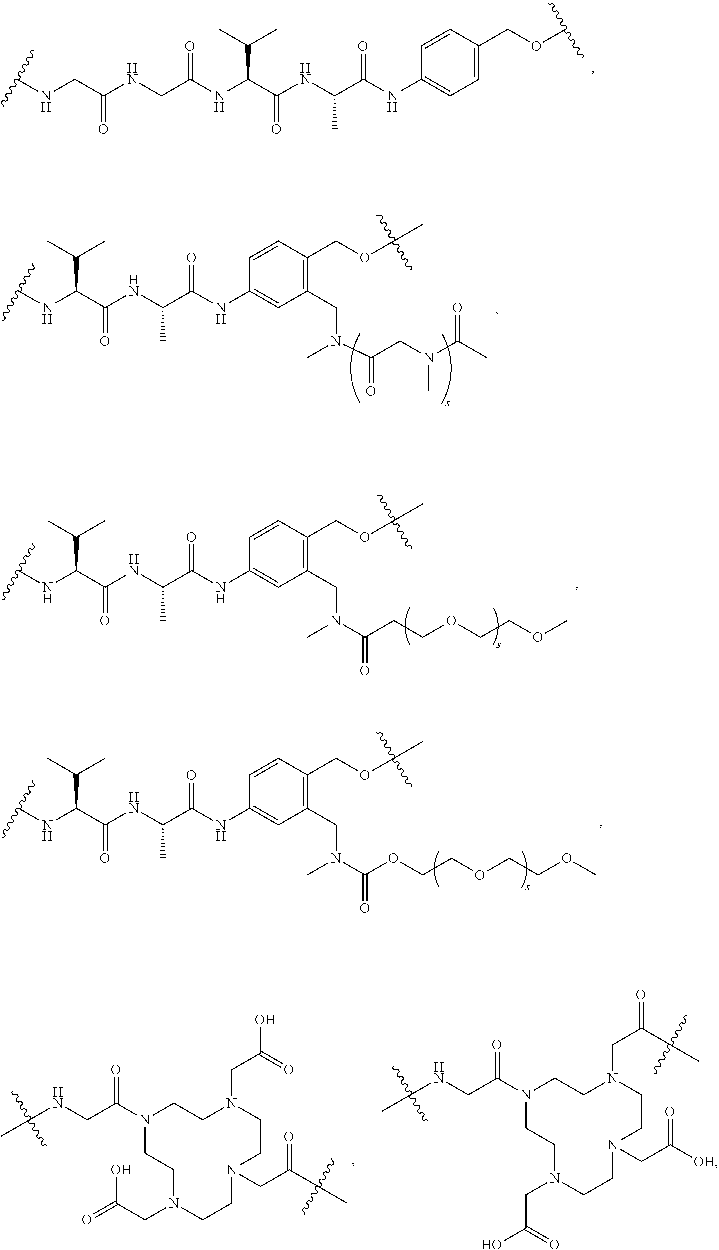

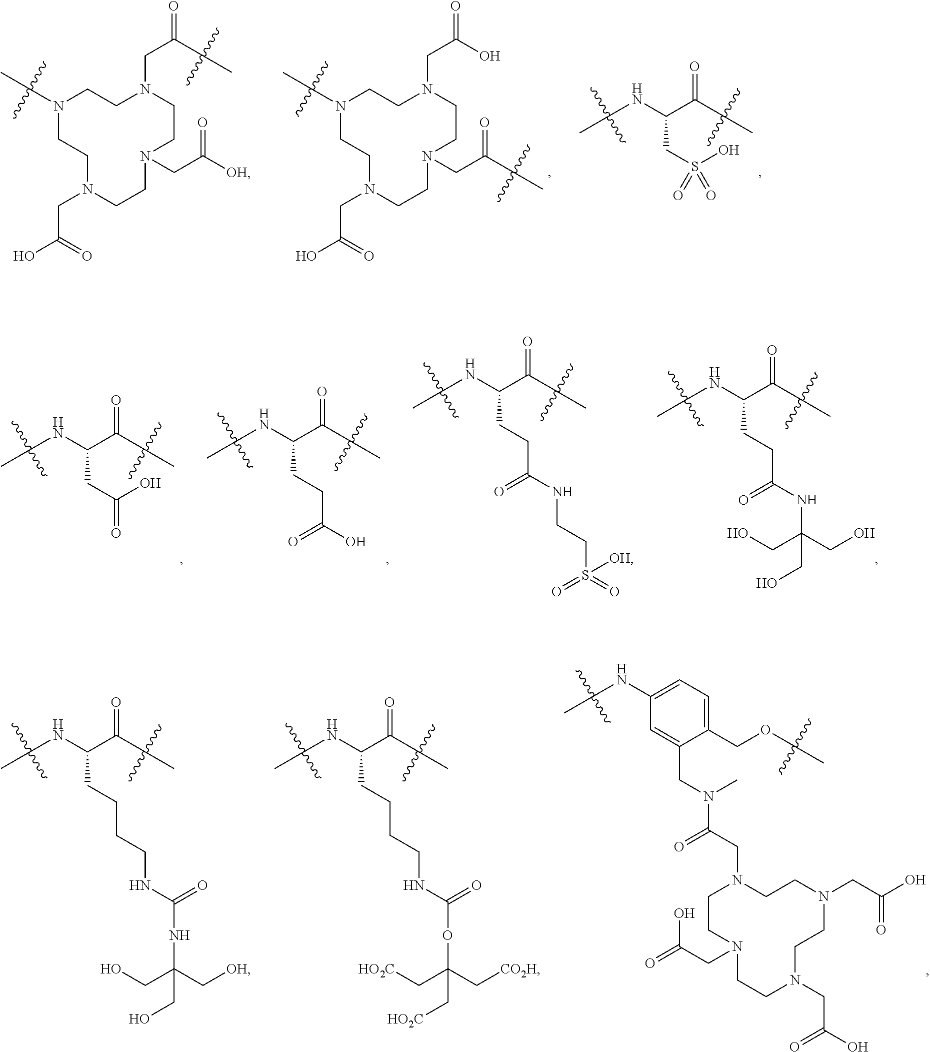

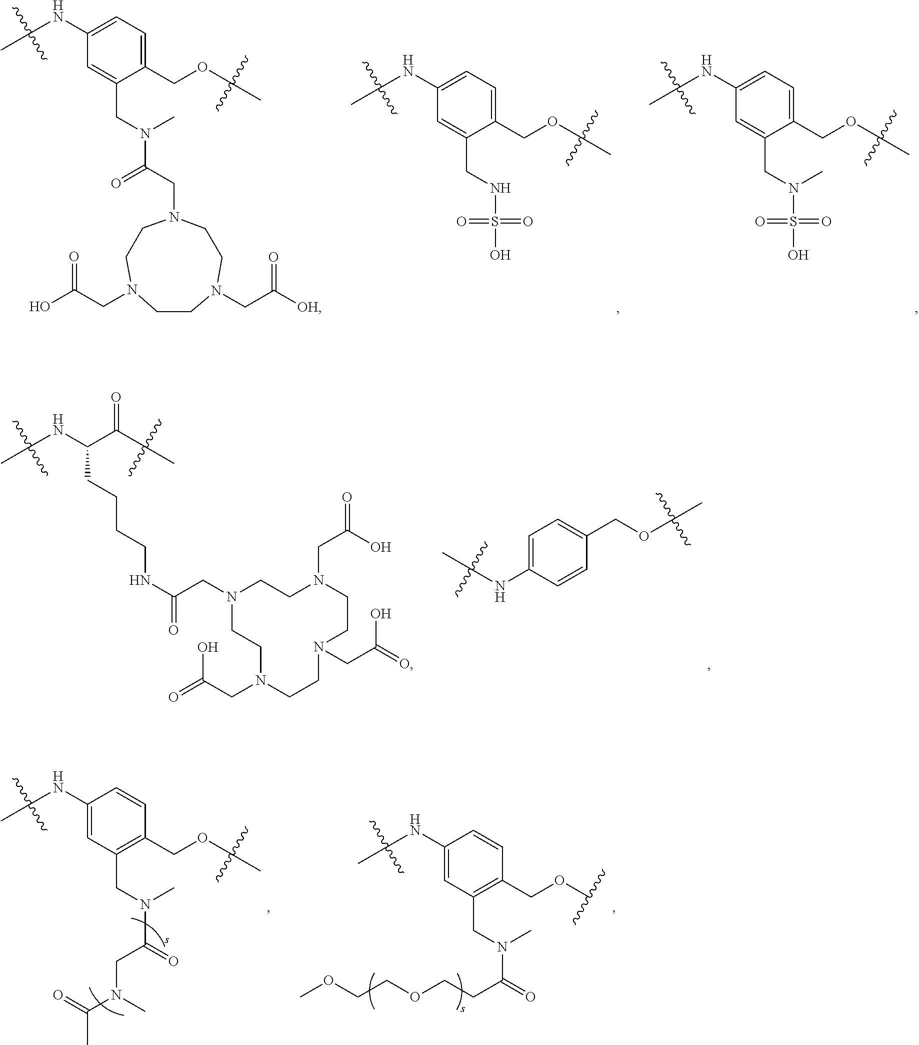

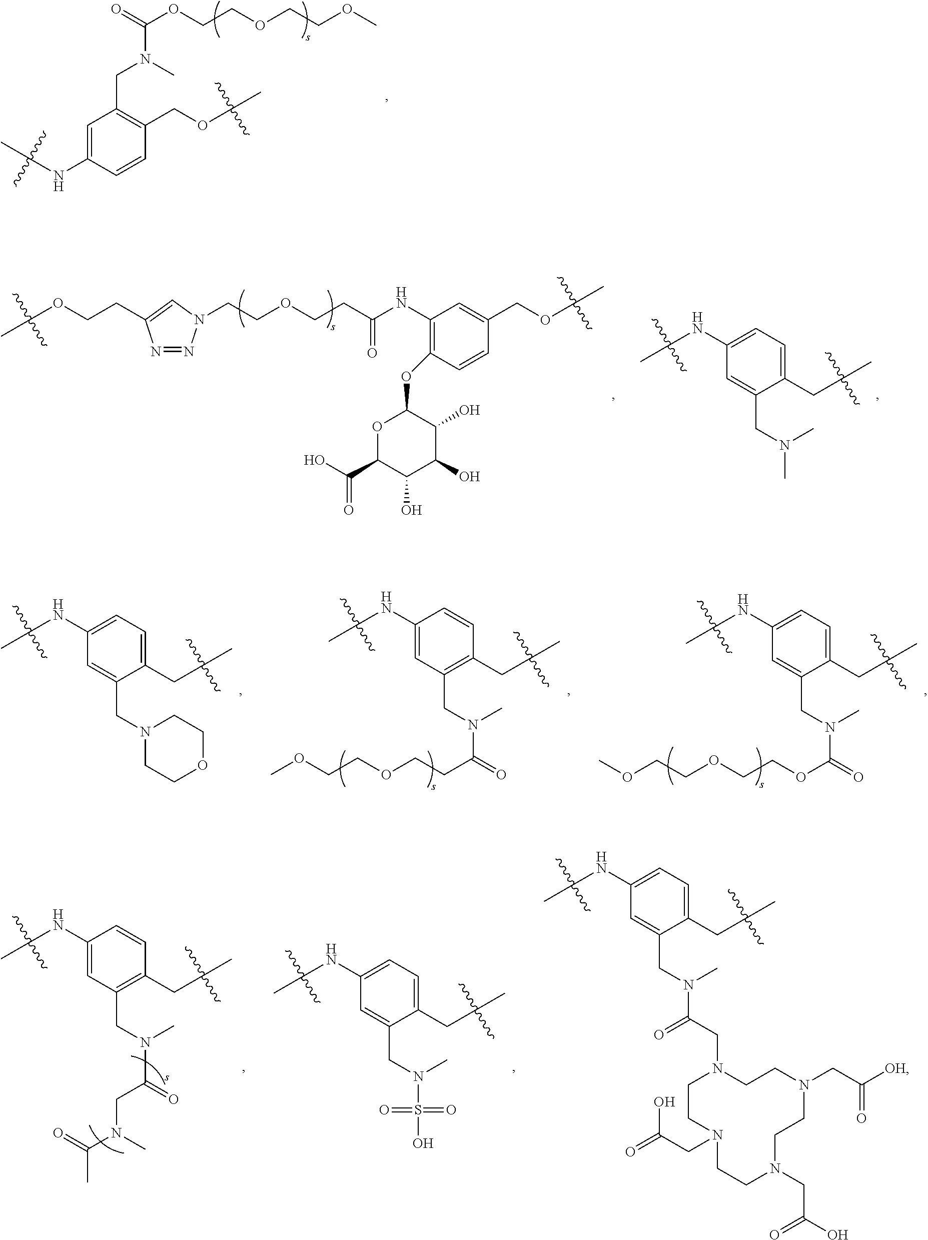

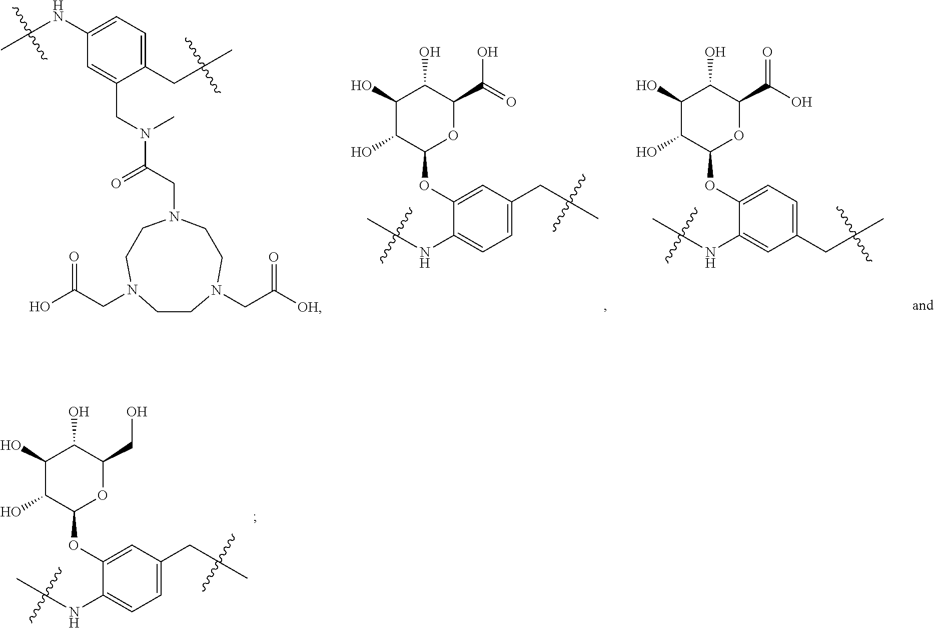 [SMILES: *Cc1ccc(N*)cc1CN(C)C(=O)CN1CCN(CC(=O)O)CCN(CC(=O)O)CC1 ; *Cc1ccc(N*)c(O[C@@H]2O[C@H](C(=O)O)[C@@H](O)[C@H](O)[C@H]2O)c1 ; *Cc1ccc(O[C@@H]2O[C@H](C(=O)O)[C@@H](O)[C@H](O)[C@H]2O)c(N*)c1 ; *Cc1ccc(N*)c(O[C@@H]2O[C@H](CO)[C@@H](O)[C@H](O)[C@H]2O)c1]

s is and integer selected from 1 to 20, preferably, s is an integer selected from 1 to 15, for example, 1 to 12, 3 to 12, for example, s is 1, 2, 3, 4, 5, 6, 7, 8, 9, 10, 11, 12, 13, 14 or 15.

In some embodiments, L is selected from the group consisting of the substituted or unsubstituted structural fragments composed of one or more of the following groups:

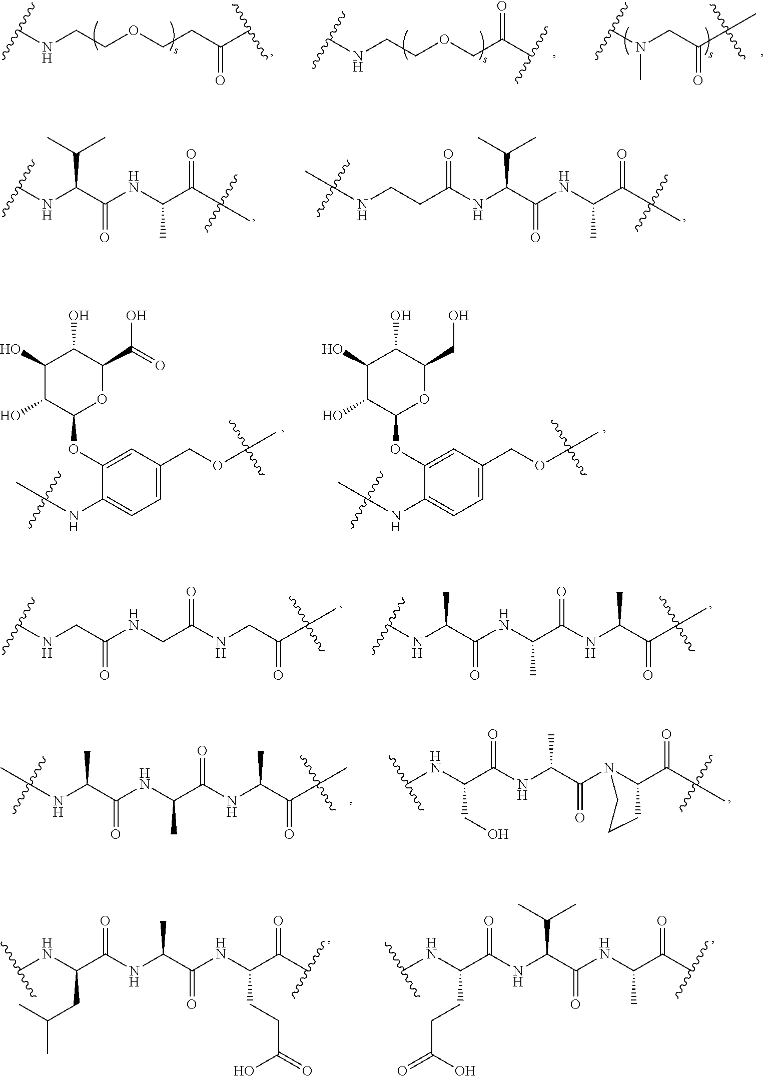

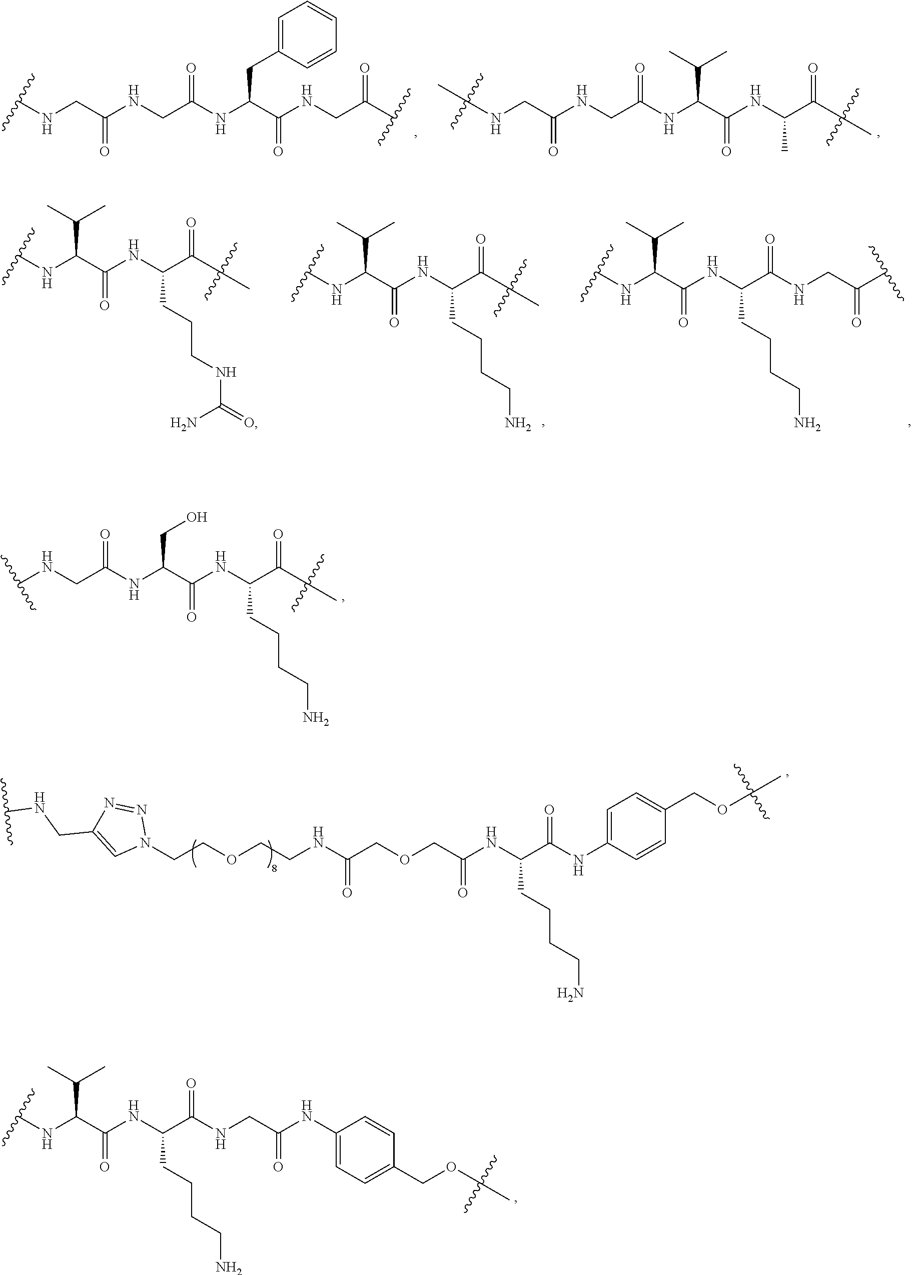

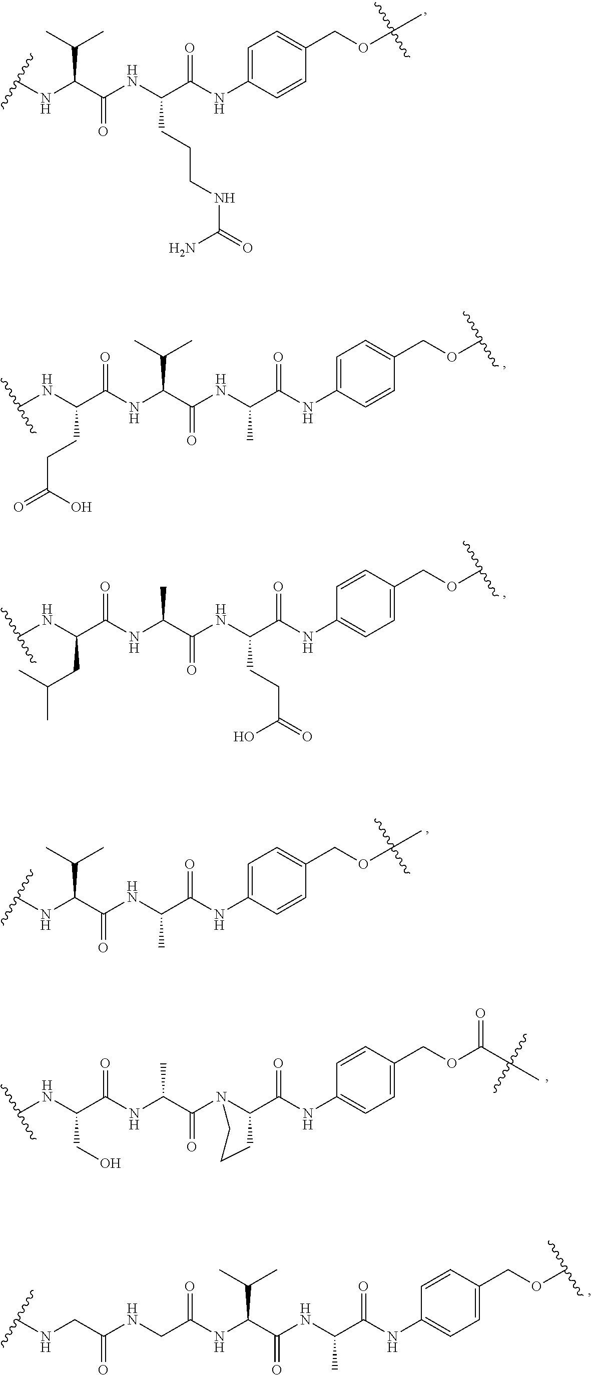

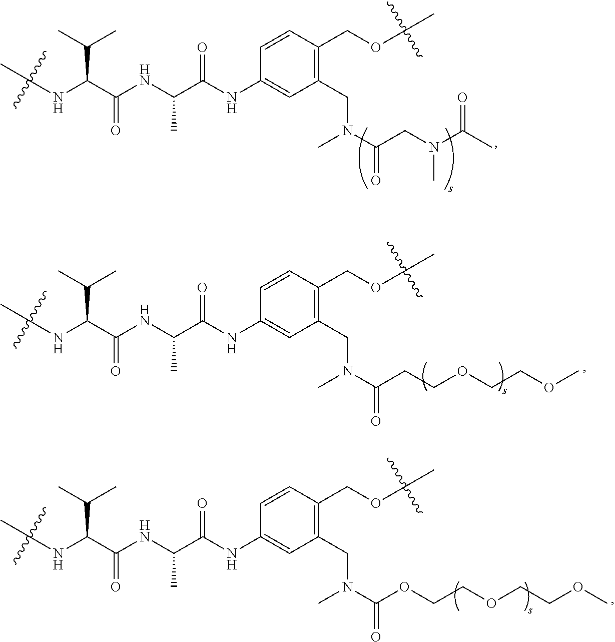

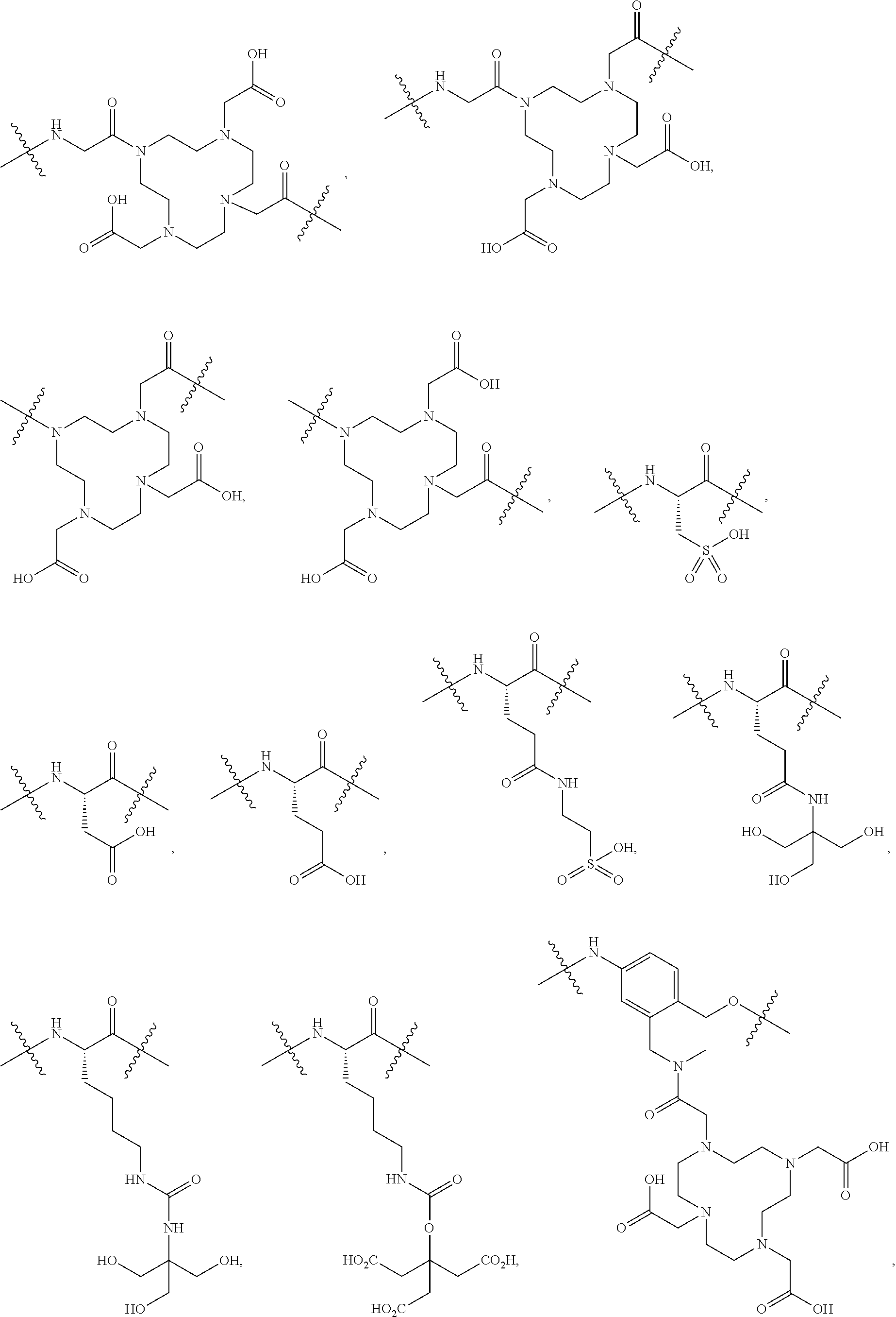

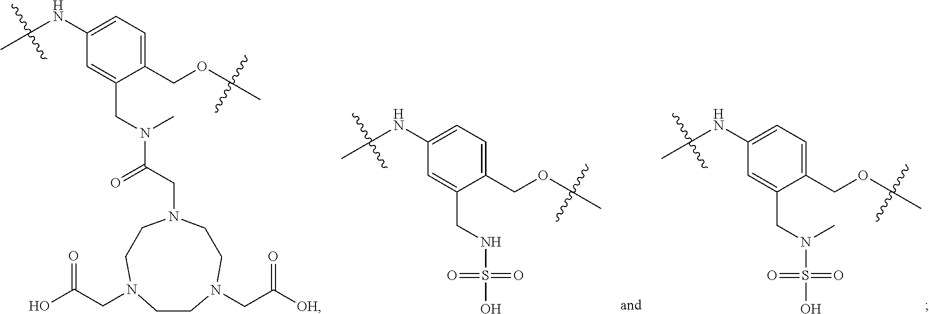 [SMILES: *Nc1ccc(CO*)c(CN(C)C(=O)CN2CCN(CC(=O)O)CCN(CC(=O)O)CC2)c1 ; *Nc1ccc(CO*)c(CNS(=O)(=O)O)c1 ; *Nc1ccc(CO*)c(CN(C)S(=O)(=O)O)c1]

s is an integer selected from 1 to 20.

In some embodiments, L is selected from the group consisting of the substituted or unsubstituted structural fragments composed of one or more of the following groups:

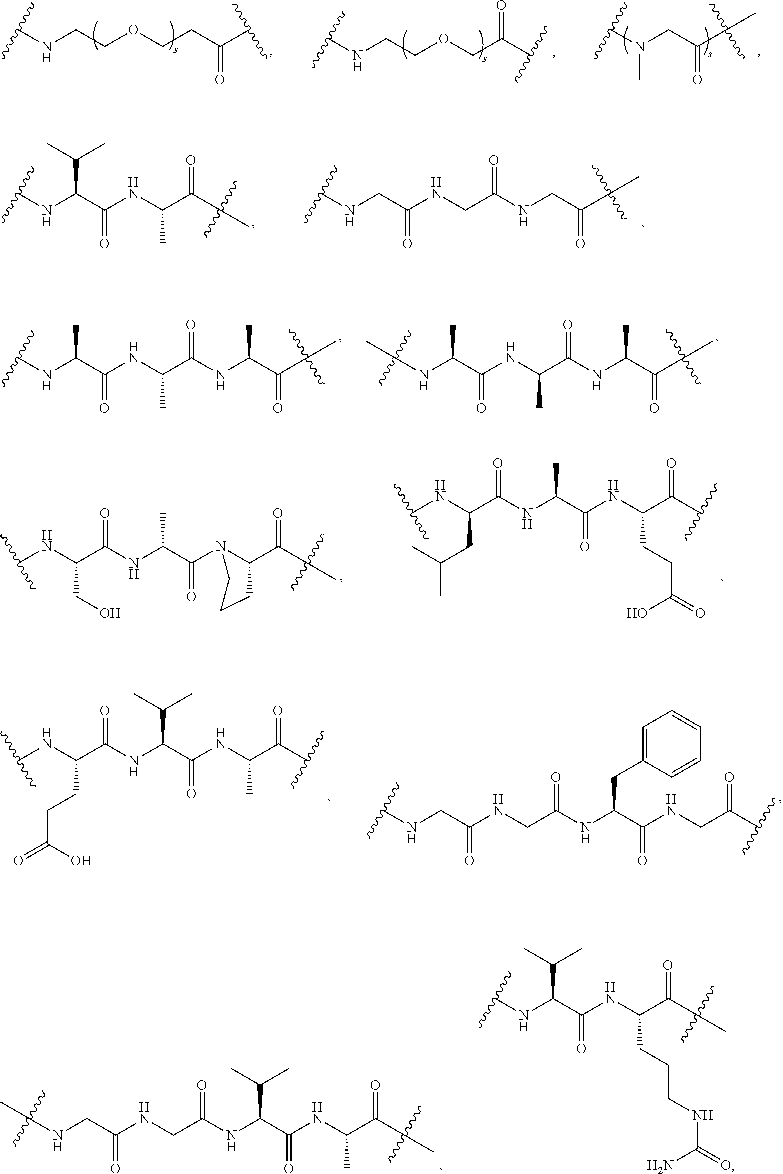

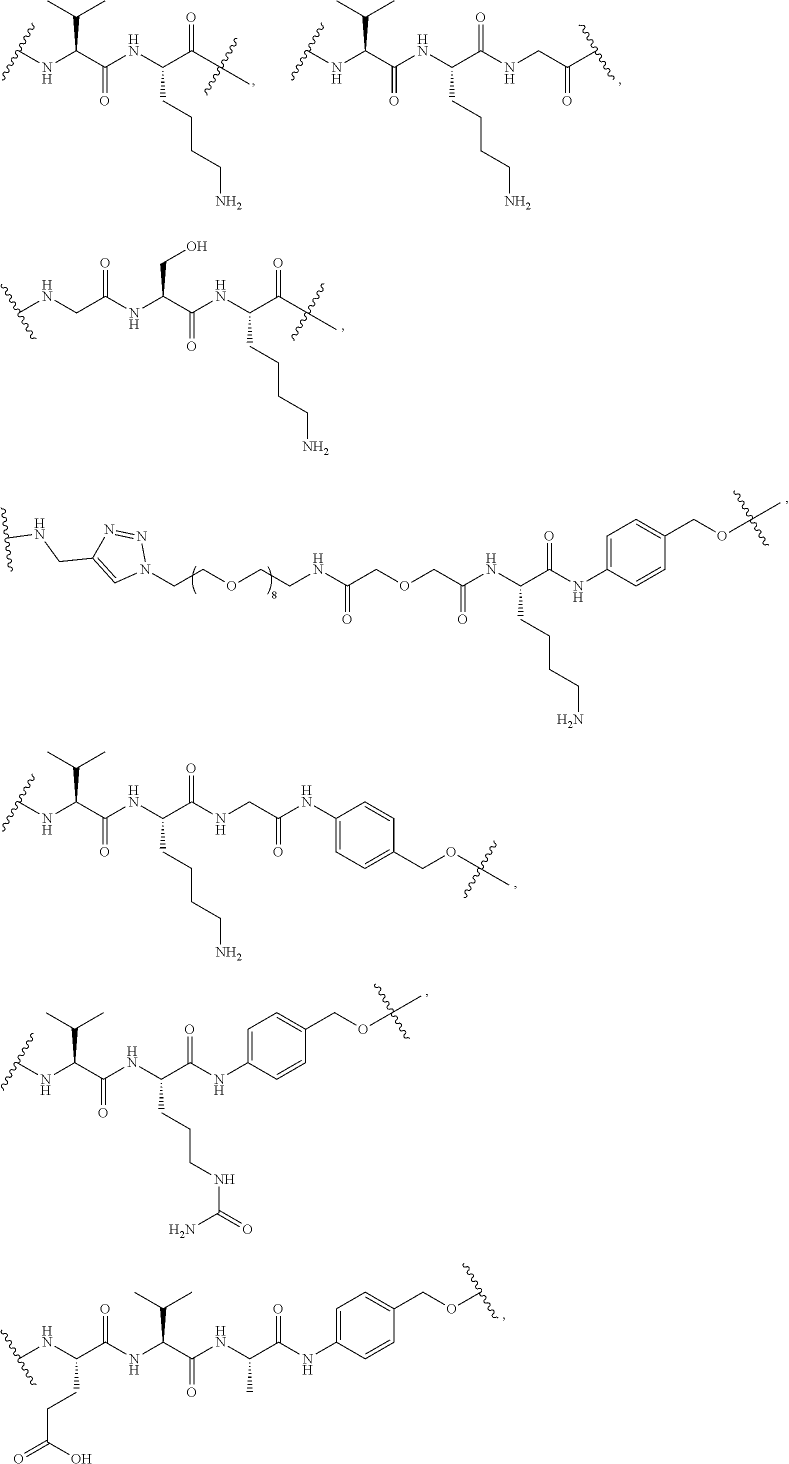

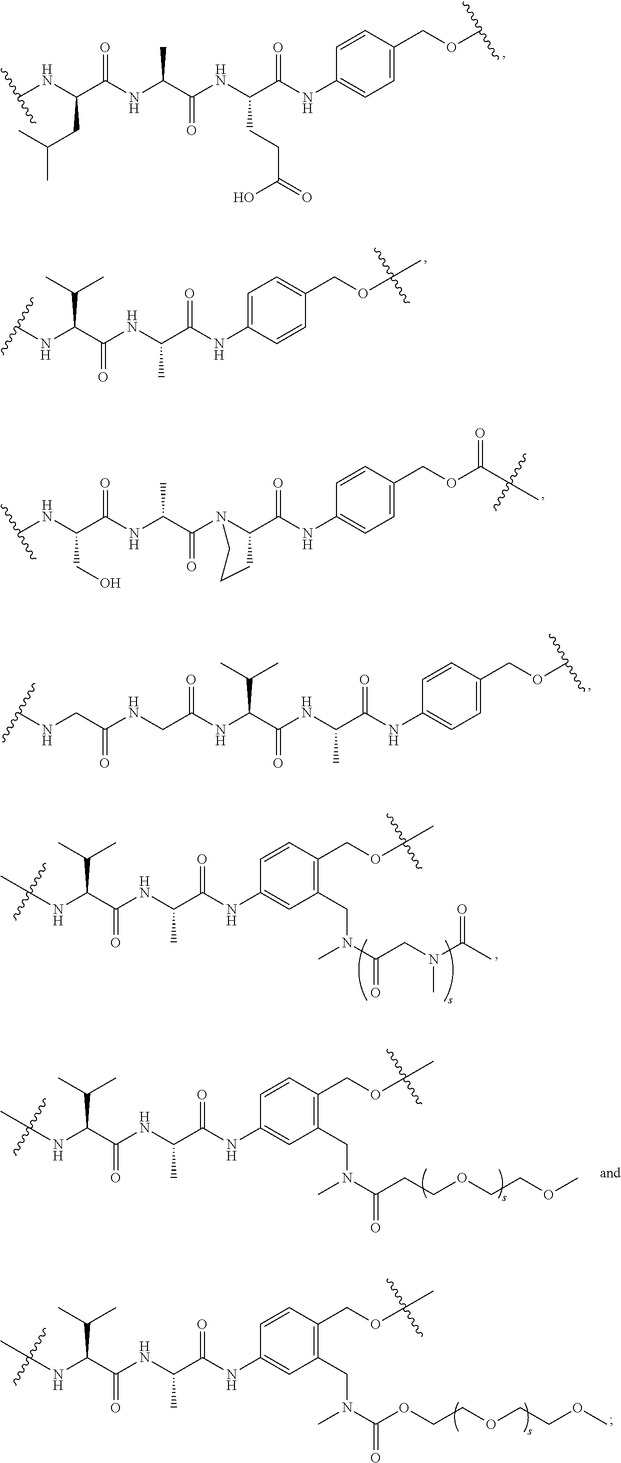

s is an integer selected from 1 to 20.

In some embodiments, L is formed by connecting one or more substituted or unsubstituted structural fragments selected from the following Group I and one or more substituted or unsubstituted structural fragments selected from the following Group IL:

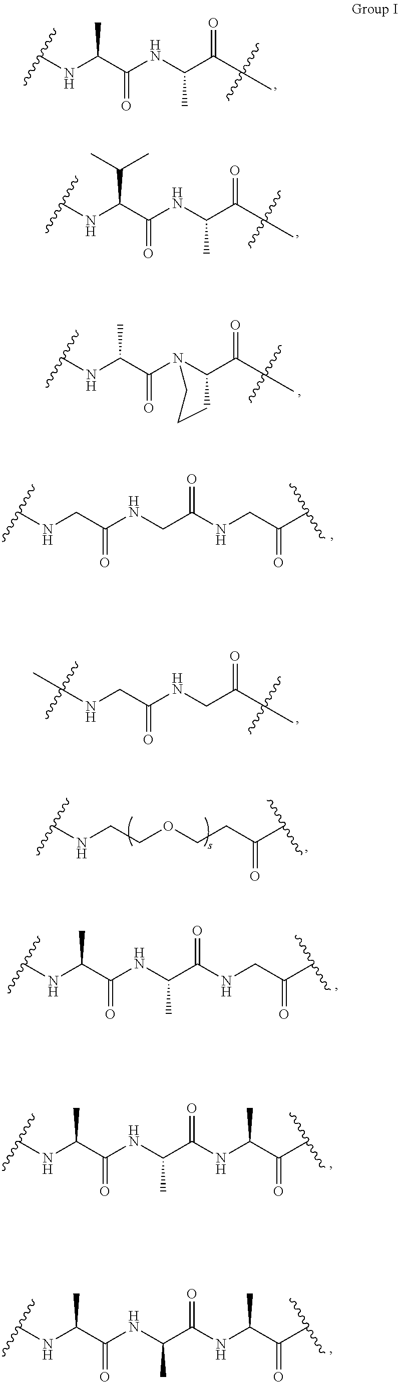

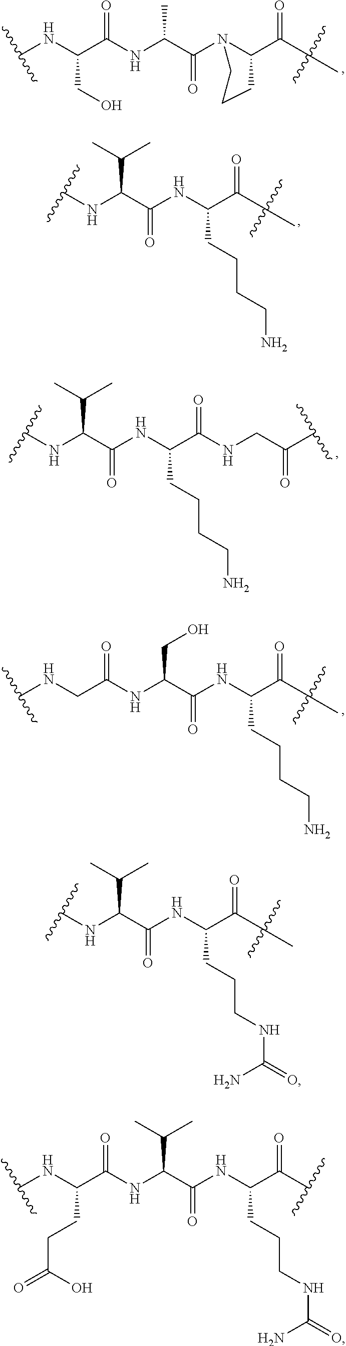

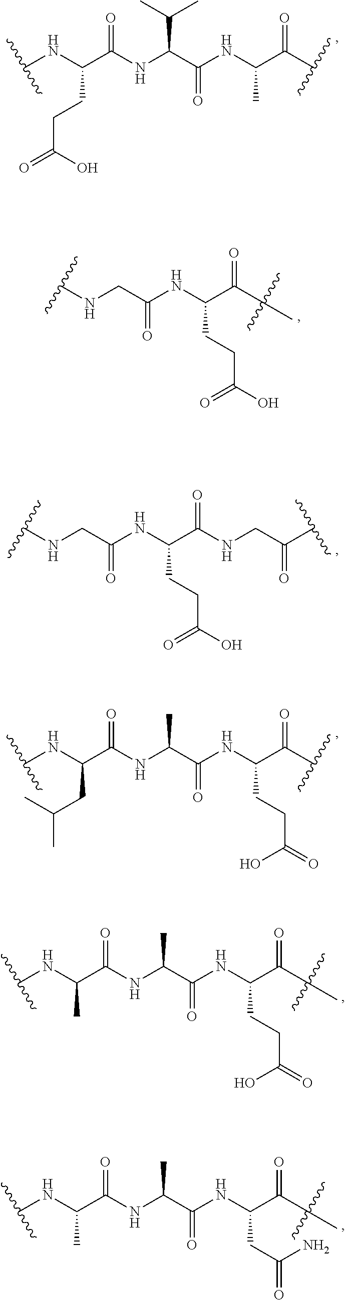

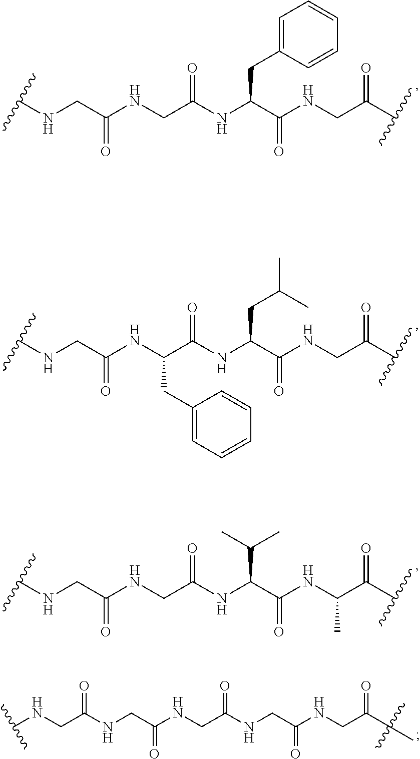

wherein s is an integer selected from 1 to 20;

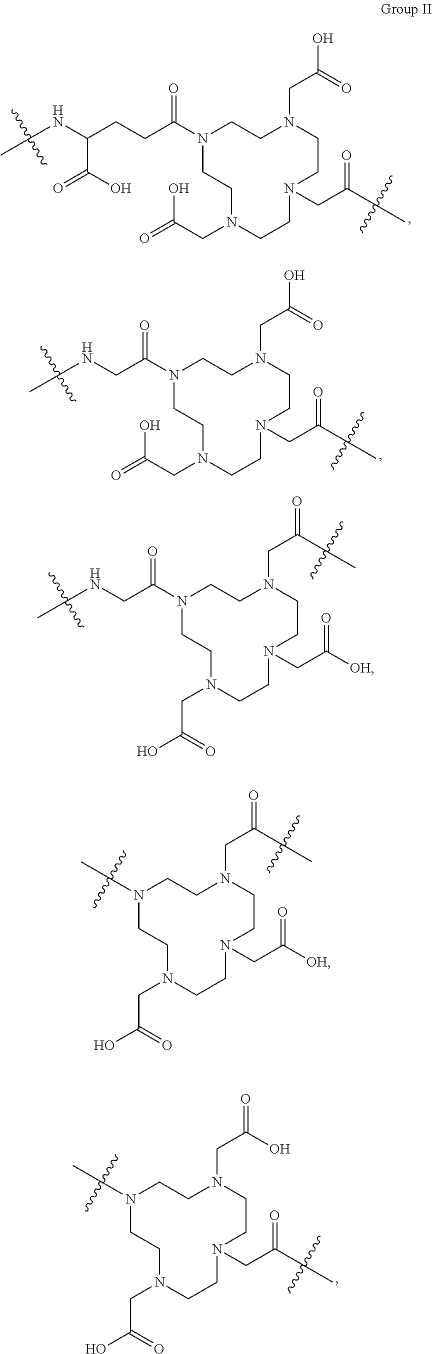

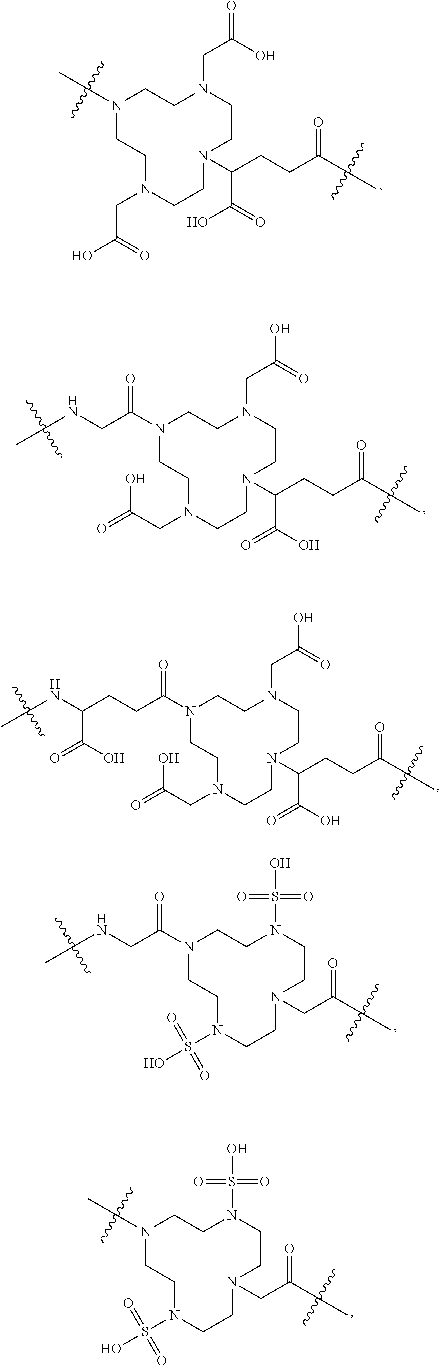

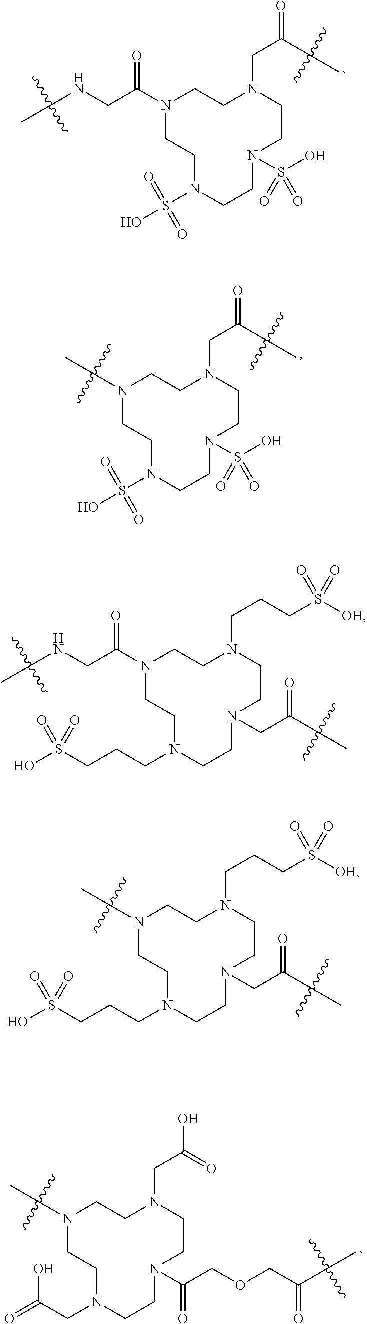

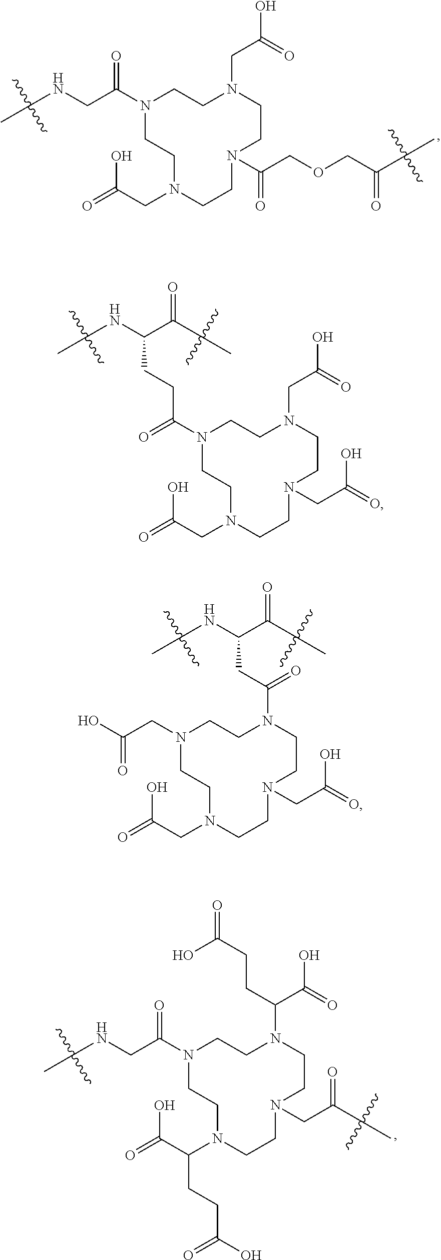 [SMILES: *NCC(=O)N1CCN(CC(=O)O)CCN(C(=O)COCC(*)=O)CCN(CC(=O)O)CC1 ; "," ; *N[C@@H](CCC(=O)N1CCN(CC(=O)O)CCN(CC(=O)O)CCN(CC(=O)O)CC1)C(*)=O ; *N[C@@H](CC(=O)N1CCN(CC(=O)O)CCN(CC(=O)O)CCN(CC(=O)O)CC1)C(*)=O ; *NCC(=O)N1CCN(C(CCC(=O)O)C(=O)O)CCN(CC(*)=O)CCN(C(CCC(=O)O)C(=O)O)CC1 ; ","]

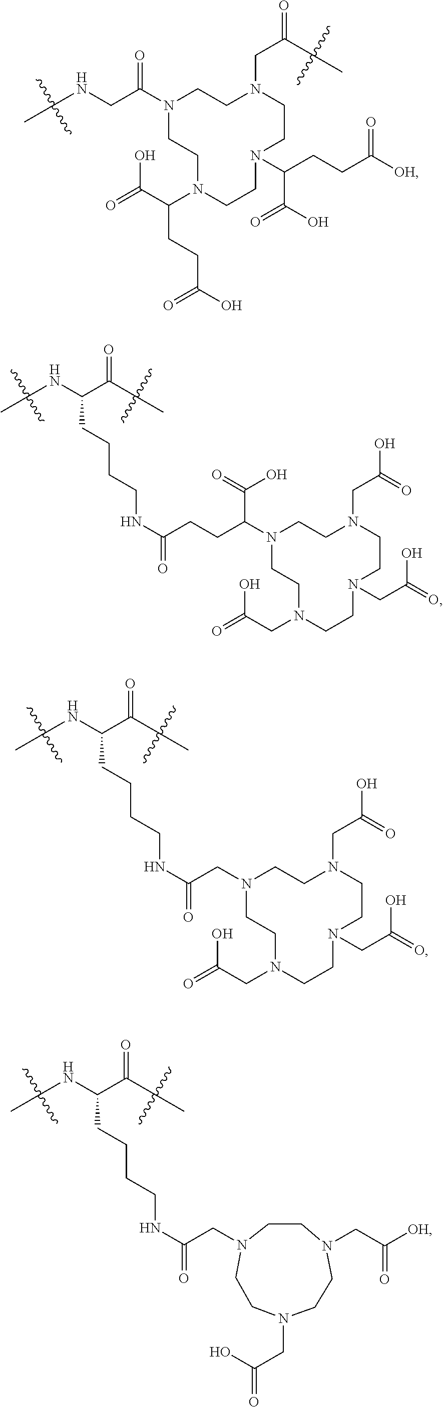 [SMILES: *NCC(=O)N1CCN(CC(*)=O)CCN(C(CCC(=O)O)C(=O)O)CCN(C(CCC(=O)O)C(=O)O)CC1 ; *N[C@@H](CCCCNC(=O)CCC(C(=O)O)N1CCN(CC(=O)O)CCN(CC(=O)O)CCN(CC(=O)O)CC1)C(*)=O ; *N[C@@H](CCCCNC(=O)CN1CCN(CC(=O)O)CCN(CC(=O)O)CCN(CC(=O)O)CC1)C(*)=O ; *N[C@@H](CCCCNC(=O)CN1CCN(CC(=O)O)CCN(CC(=O)O)CC1)C(*)=O]

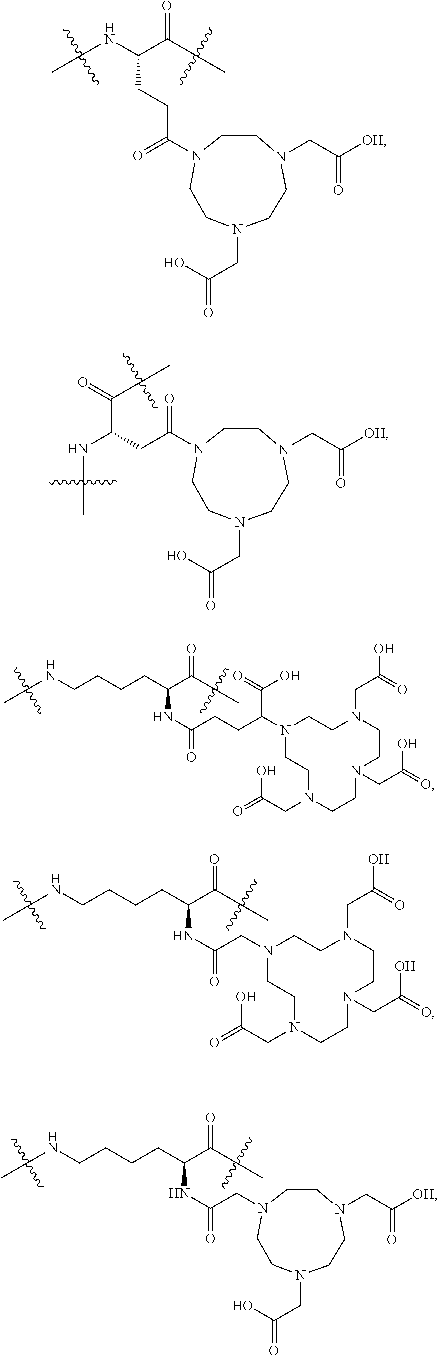 [SMILES: *N[C@@H](CCC(=O)N1CCN(CC(=O)O)CCN(CC(=O)O)CC1)C(*)=O ; *N[C@@H](CC(=O)N1CCN(CC(=O)O)CCN(CC(=O)O)CC1)C(*)=O ; *NCCCC[C@H](NC(=O)CCC(C(=O)O)N1CCN(CC(=O)O)CCN(CC(=O)O)CCN(CC(=O)O)CC1)C(*)=O ; *NCCCC[C@H](NC(=O)CN1CCN(CC(=O)O)CCN(CC(=O)O)CCN(CC(=O)O)CC1)C(*)=O ; *NCCCC[C@H](NC(=O)CN1CCN(CC(=O)O)CCN(CC(=O)O)CC1)C(*)=O]

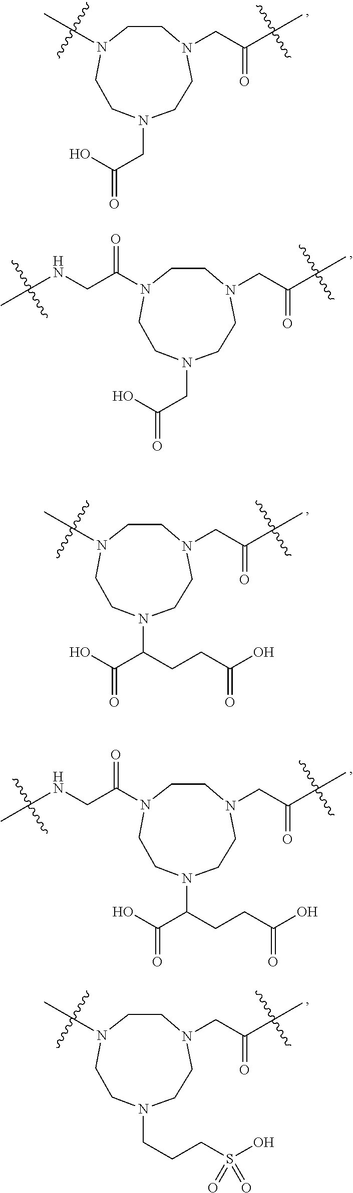

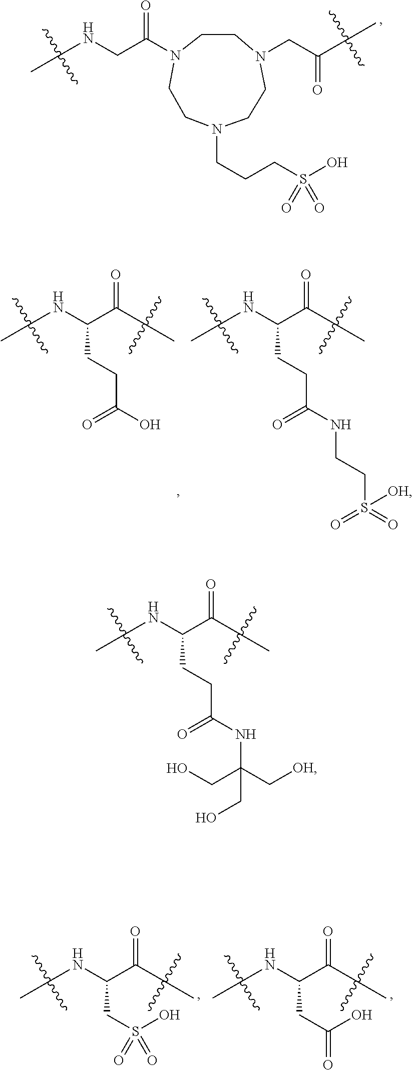

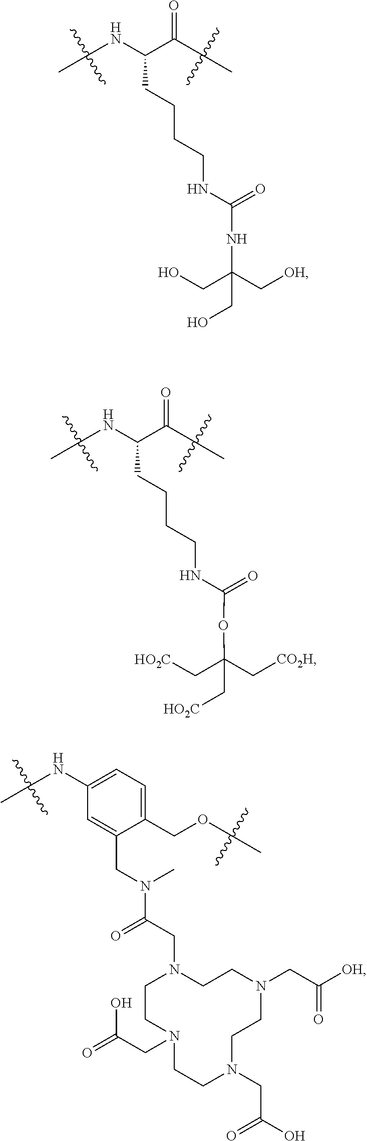 [SMILES: *N[C@@H](CCCCNC(=O)NC(CO)(CO)CO)C(*)=O ; *N[C@@H](CCCCNC(=O)OC(CC(=O)O)(CC(=O)O)CC(=O)O)C(*)=O ; *Nc1ccc(CO*)c(CN(C)C(=O)CN2CCN(CC(=O)O)CCN(CC(=O)O)CCN(CC(=O)O)CC2)c1]

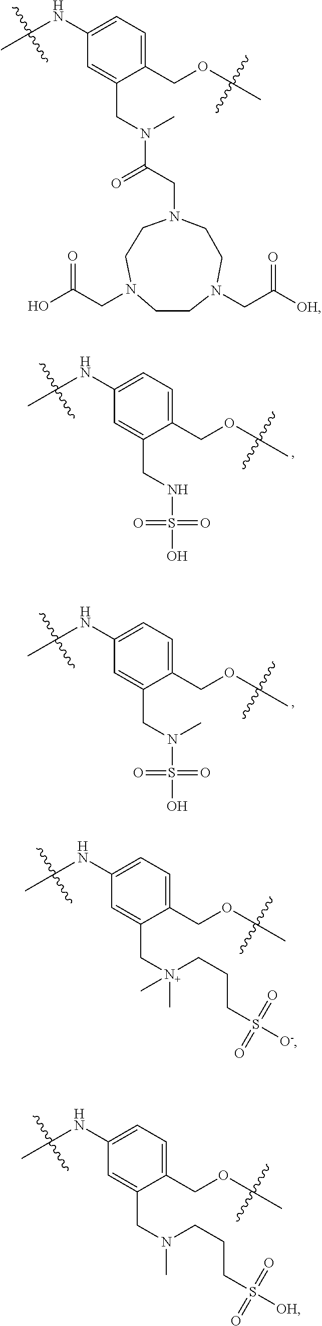

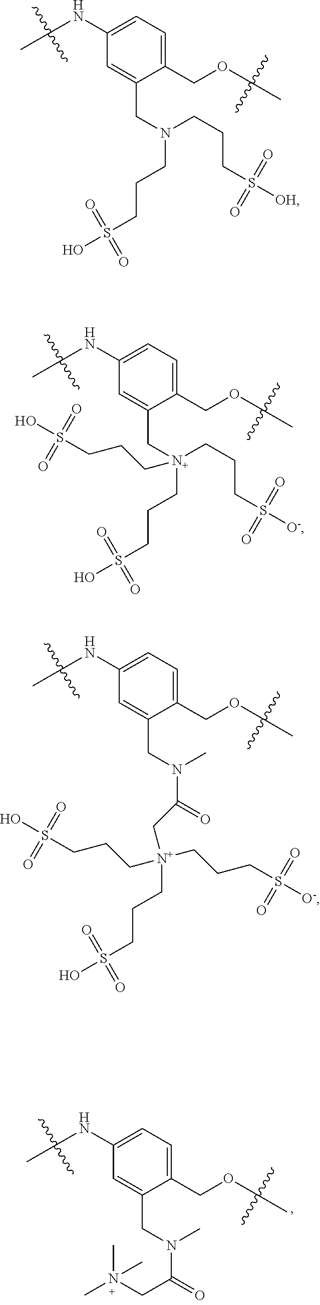

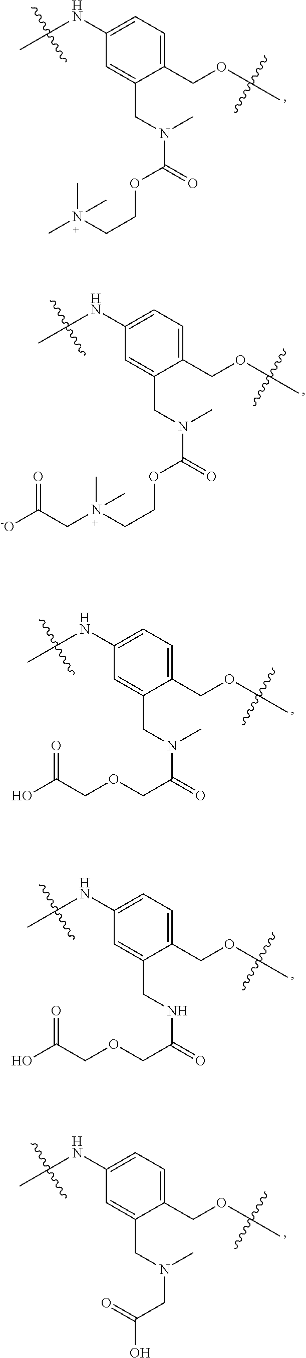

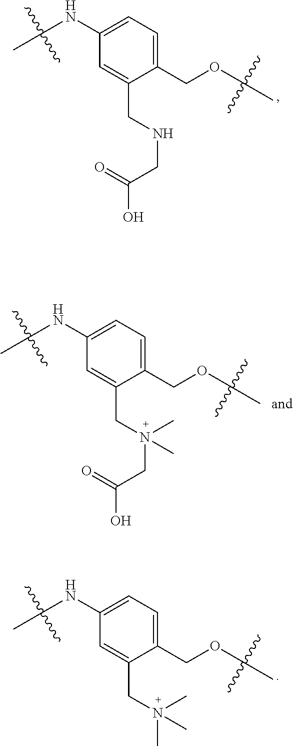

In some embodiments, L is selected from the group consisting of the substituted or unsubstituted structural fragments composed of one or more of the following groups:

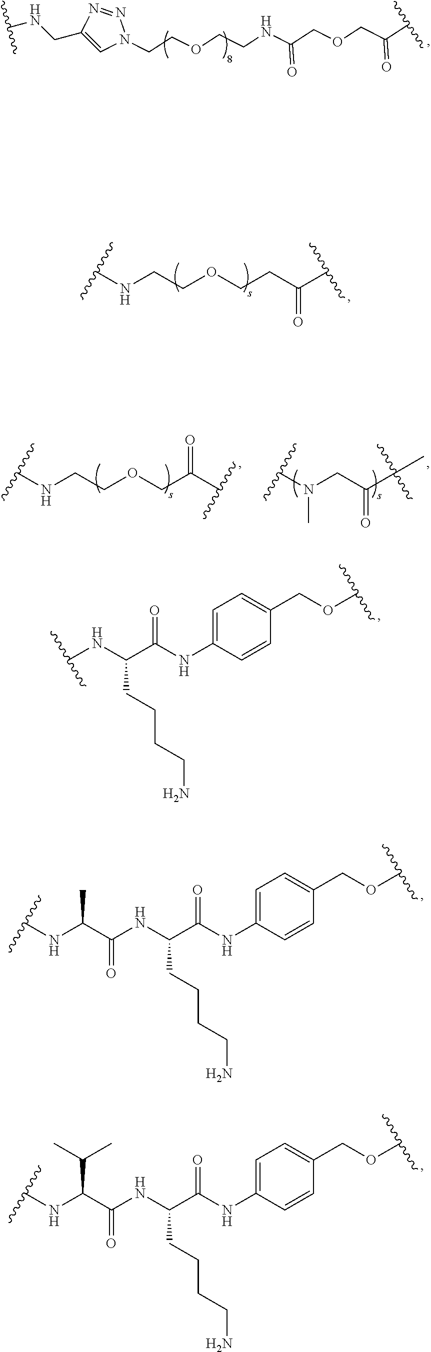

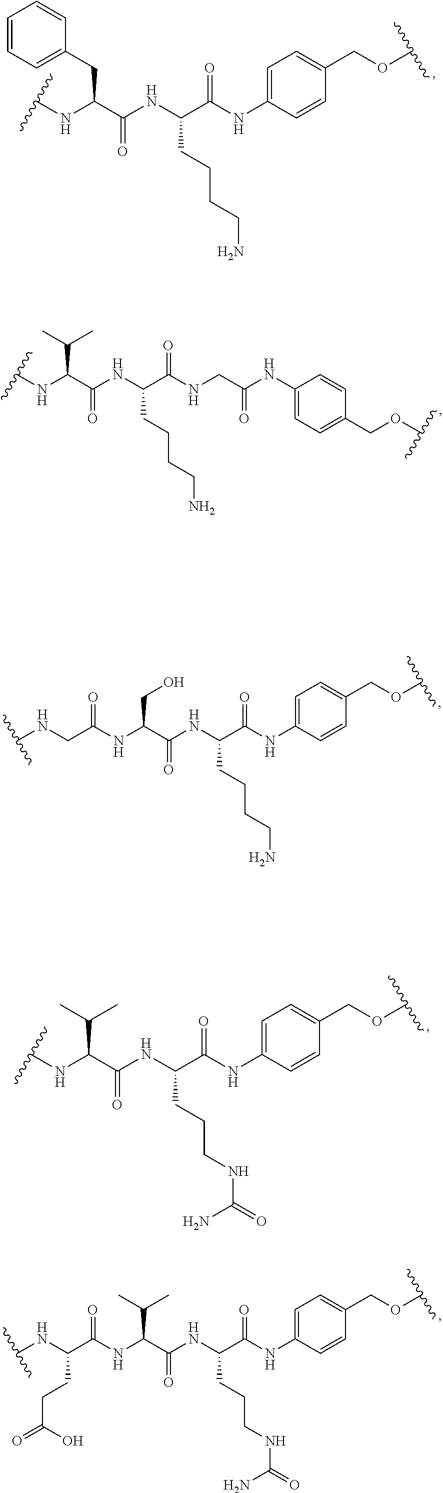

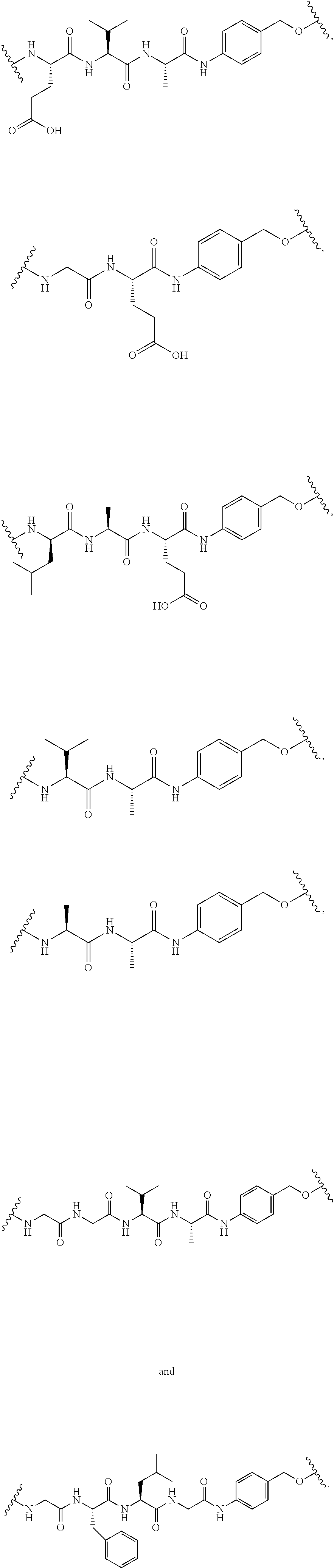

In some embodiments, L comprises one or more of the following groups:

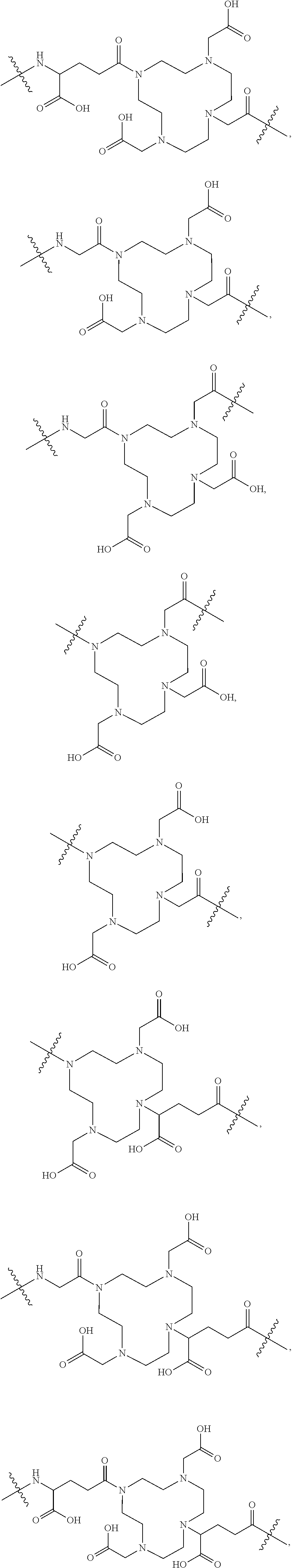

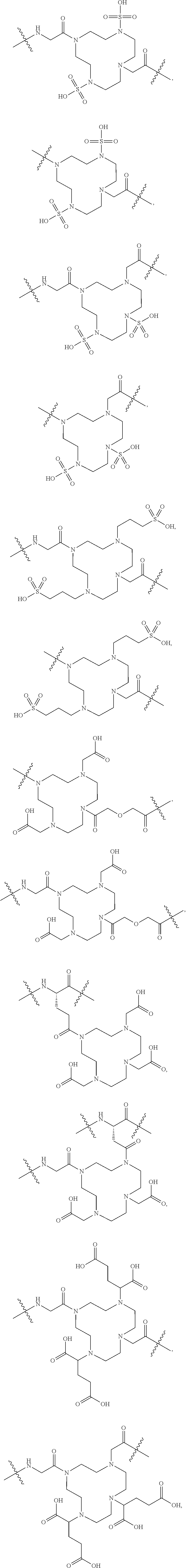

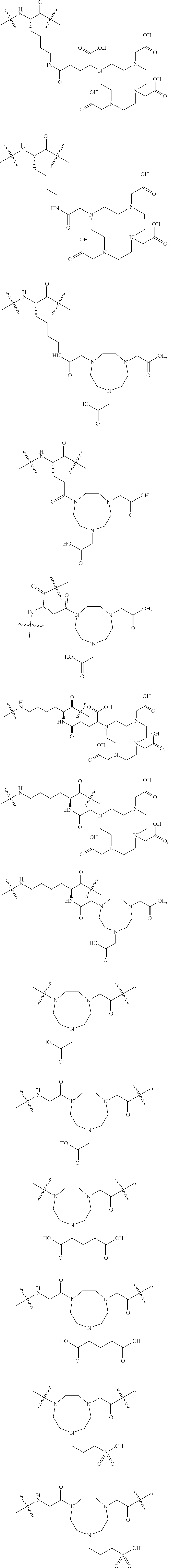

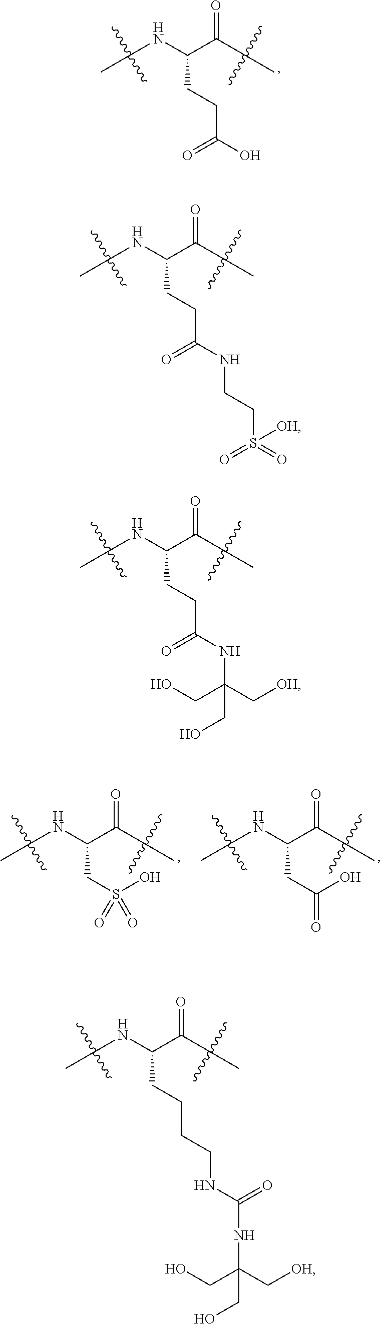 [SMILES: *N[C@@H](CCC(=O)O)C(*)=O ; *N[C@@H](CCC(=O)NCCS(=O)(=O)O)C(*)=O ; *N[C@@H](CCC(=O)NC(CO)(CO)CO)C(*)=O ; *N[C@@H](CS(=O)(=O)O)C(*)=O ; *N[C@@H](CC(=O)O)C(*)=O ; *N[C@@H](CCCCNC(=O)NC(CO)(CO)CO)C(*)=O]

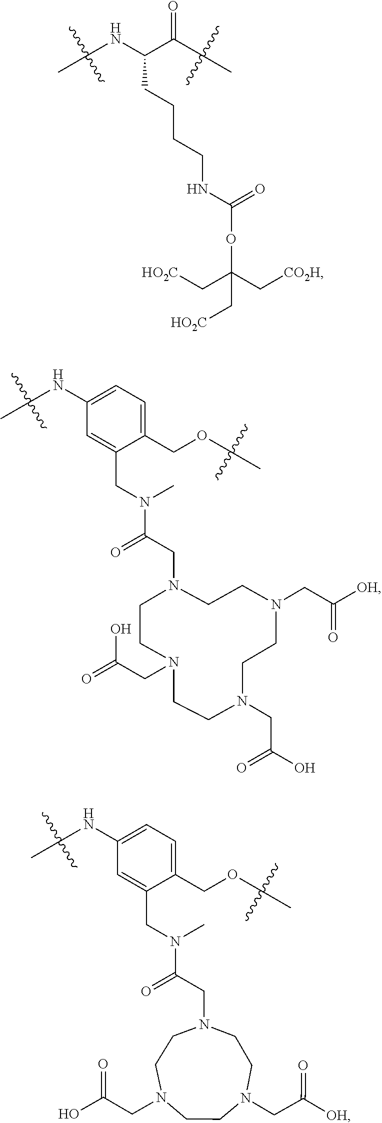 [SMILES: *N[C@@H](CCCCNC(=O)OC(CC(=O)O)(CC(=O)O)CC(=O)O)C(*)=O ; *Nc1ccc(CO*)c(CN(C)C(=O)CN2CCN(CC(=O)O)CCN(CC(=O)O)CCN(CC(=O)O)CC2)c1 ; *Nc1ccc(CO*)c(CN(C)C(=O)CN2CCN(CC(=O)O)CCN(CC(=O)O)CC2)c1]

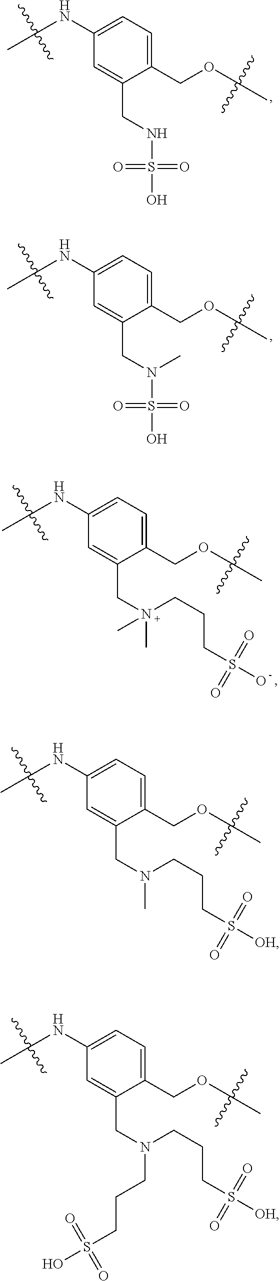

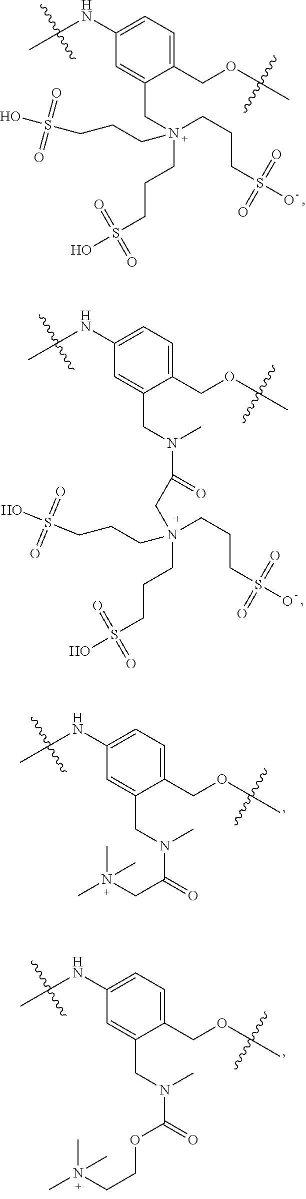

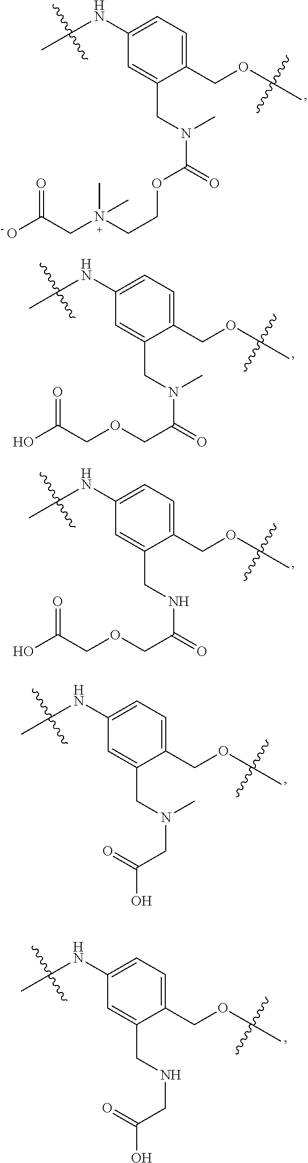

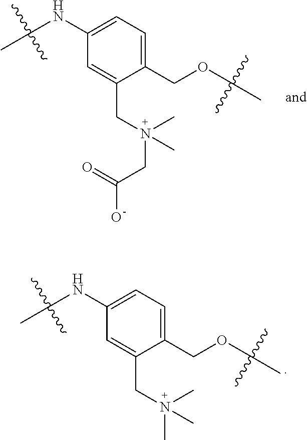

In some embodiments, L is selected from the group consisting of the substituted or unsubstituted structural fragments composed of one or more of the following groups:

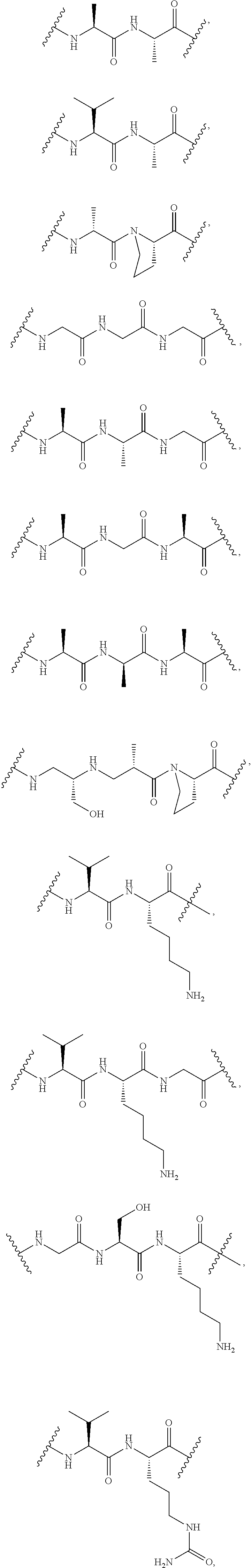

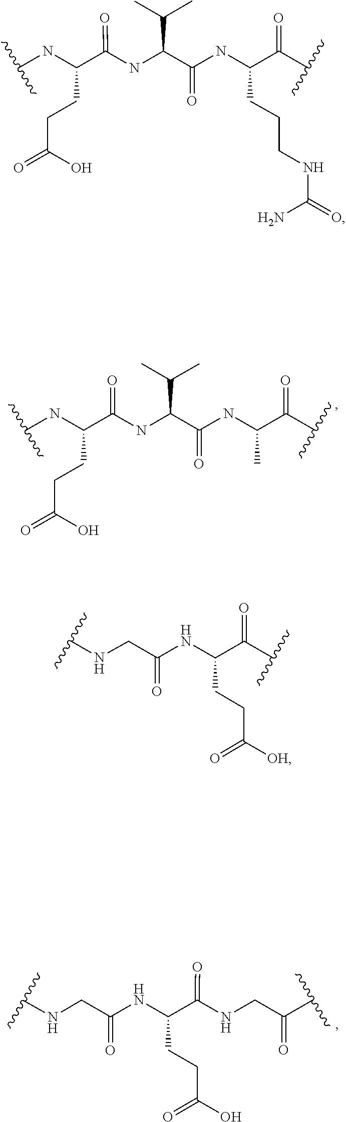

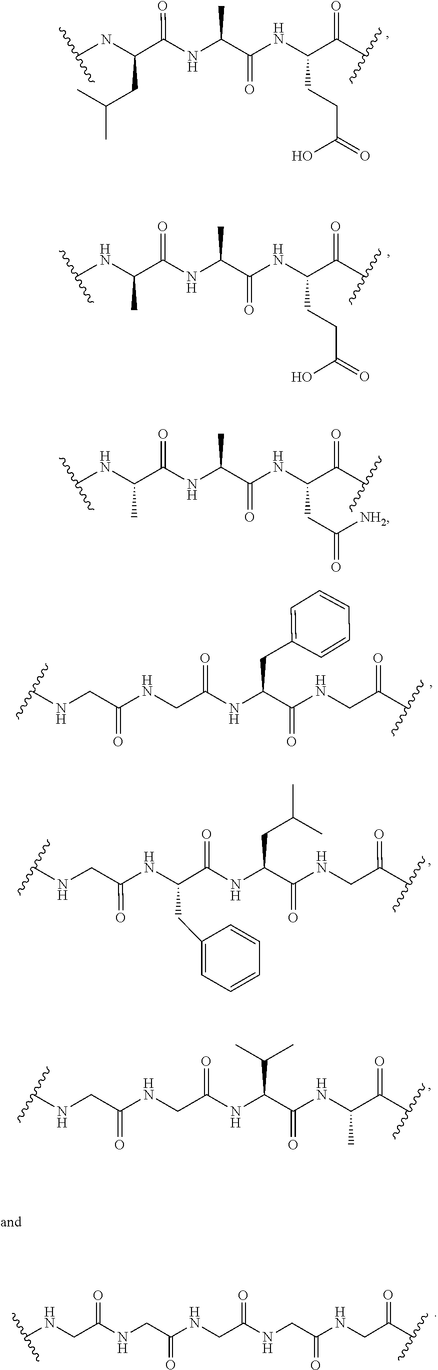

In some embodiments, L is selected from the group consisting of the following substituted or unsubstituted structural fragments:

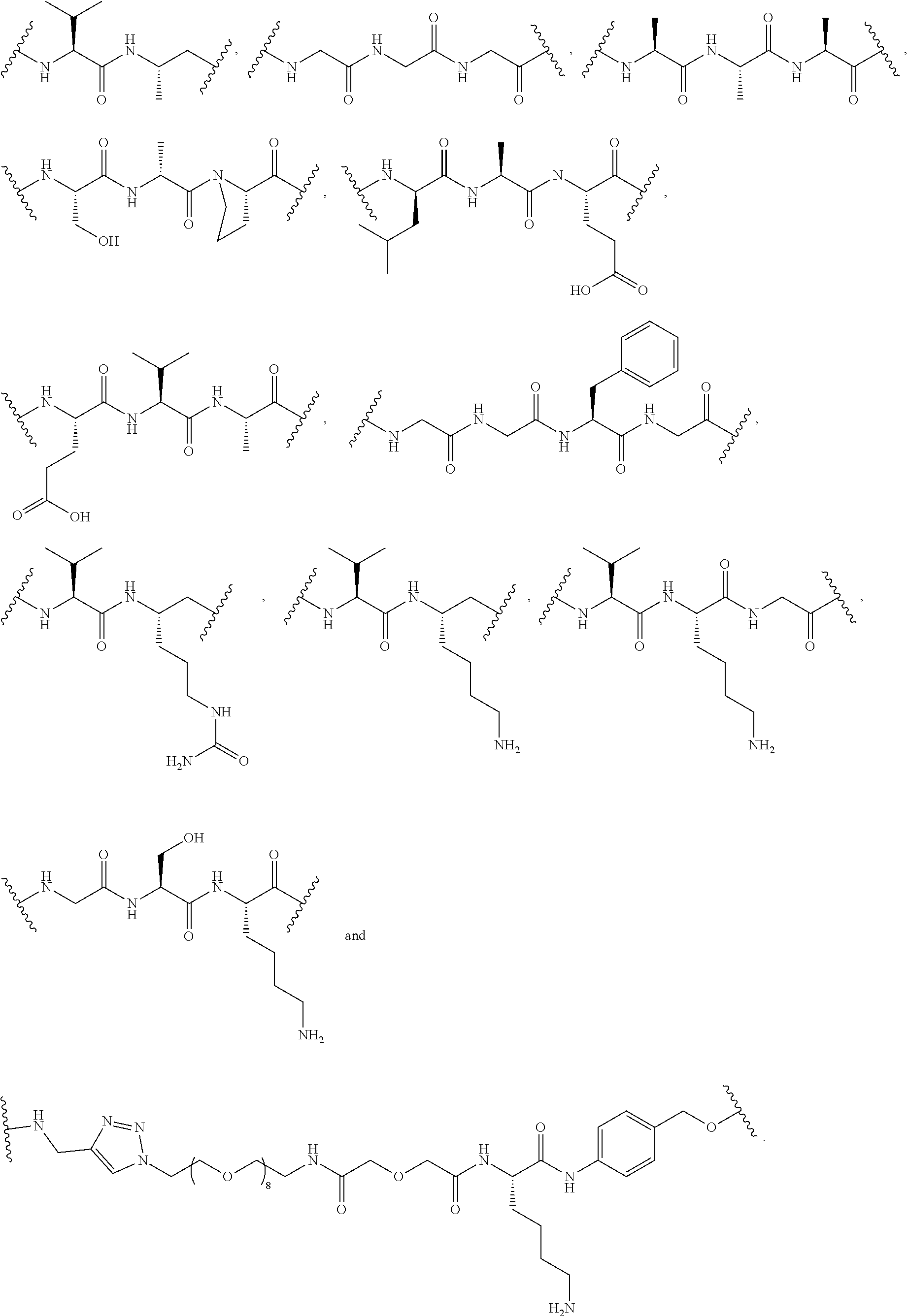

In some embodiments, L is selected from the group consisting of the substituted or unsubstituted structural fragments composed of one or more of the following groups:

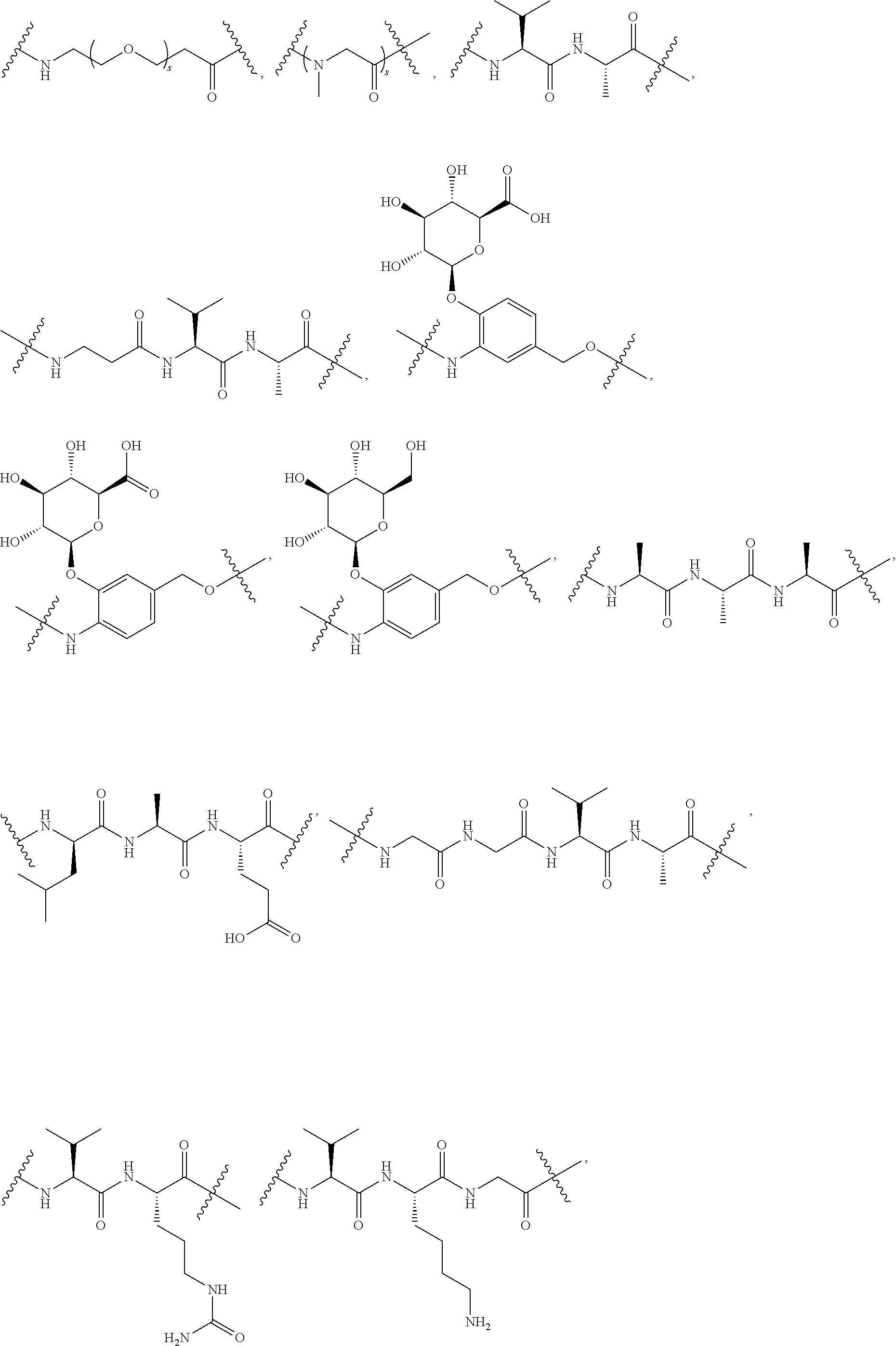

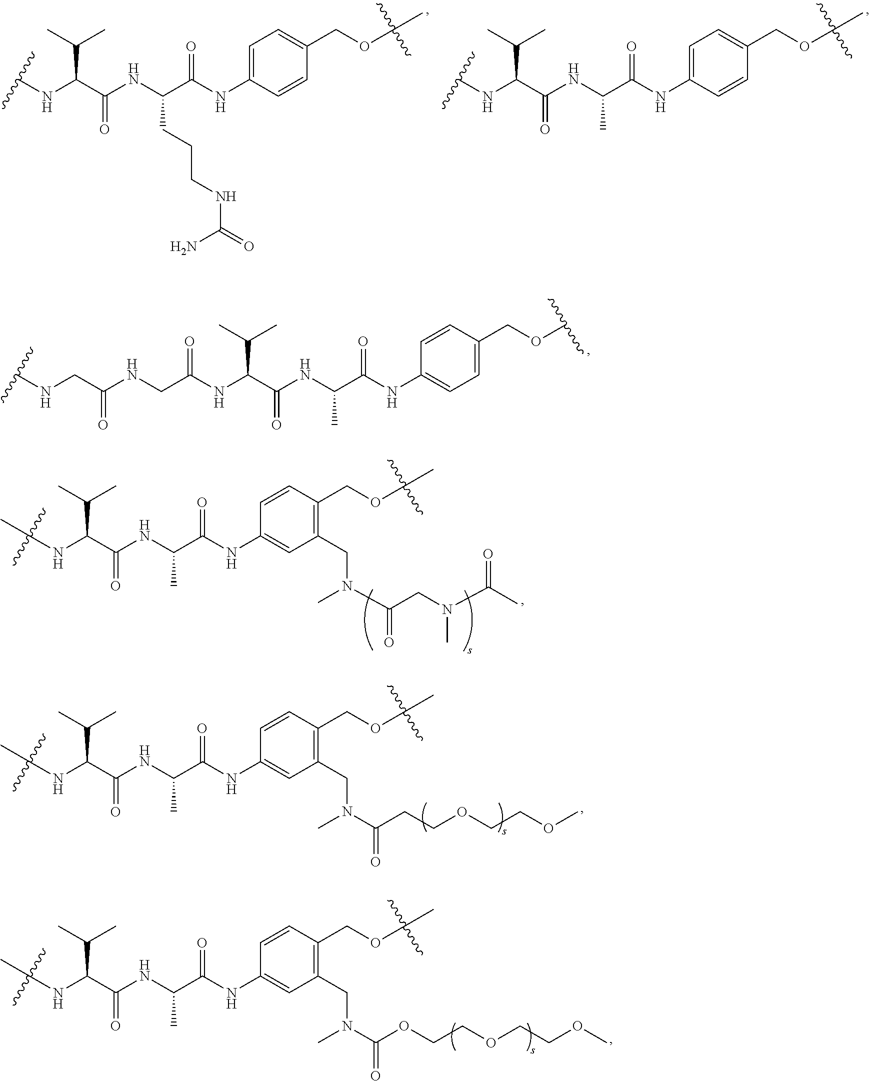

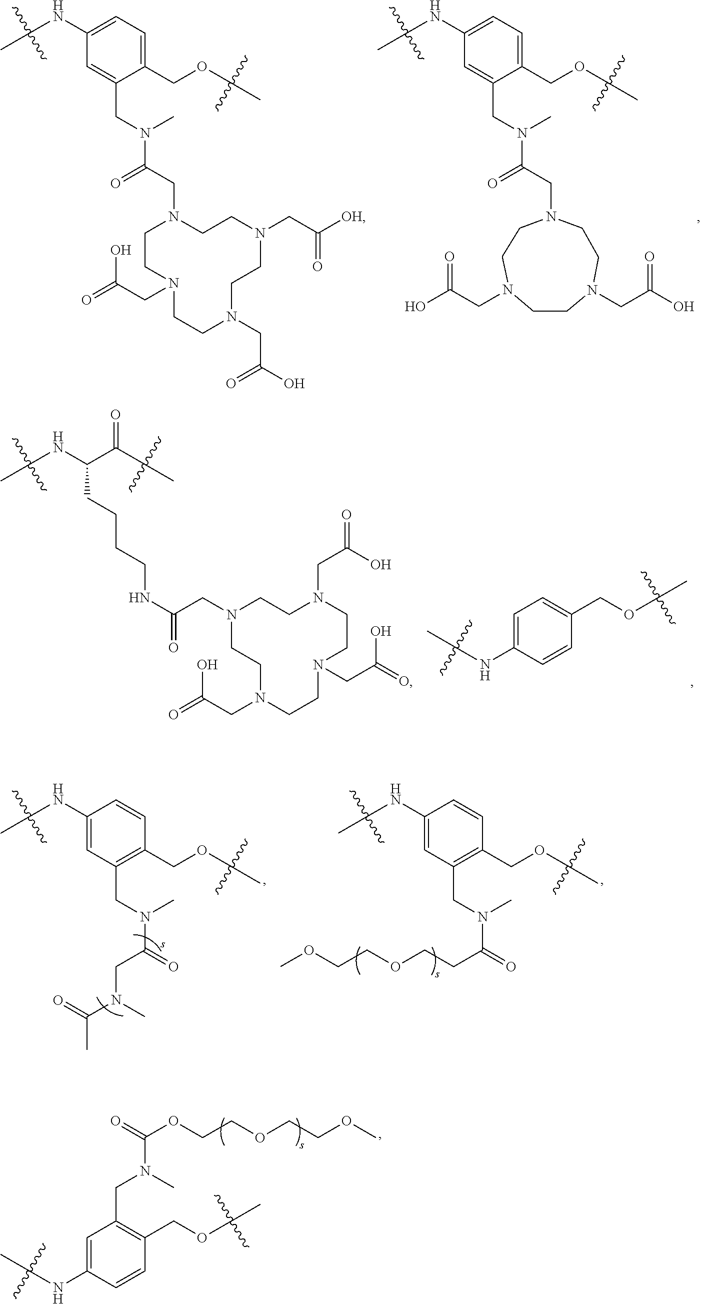

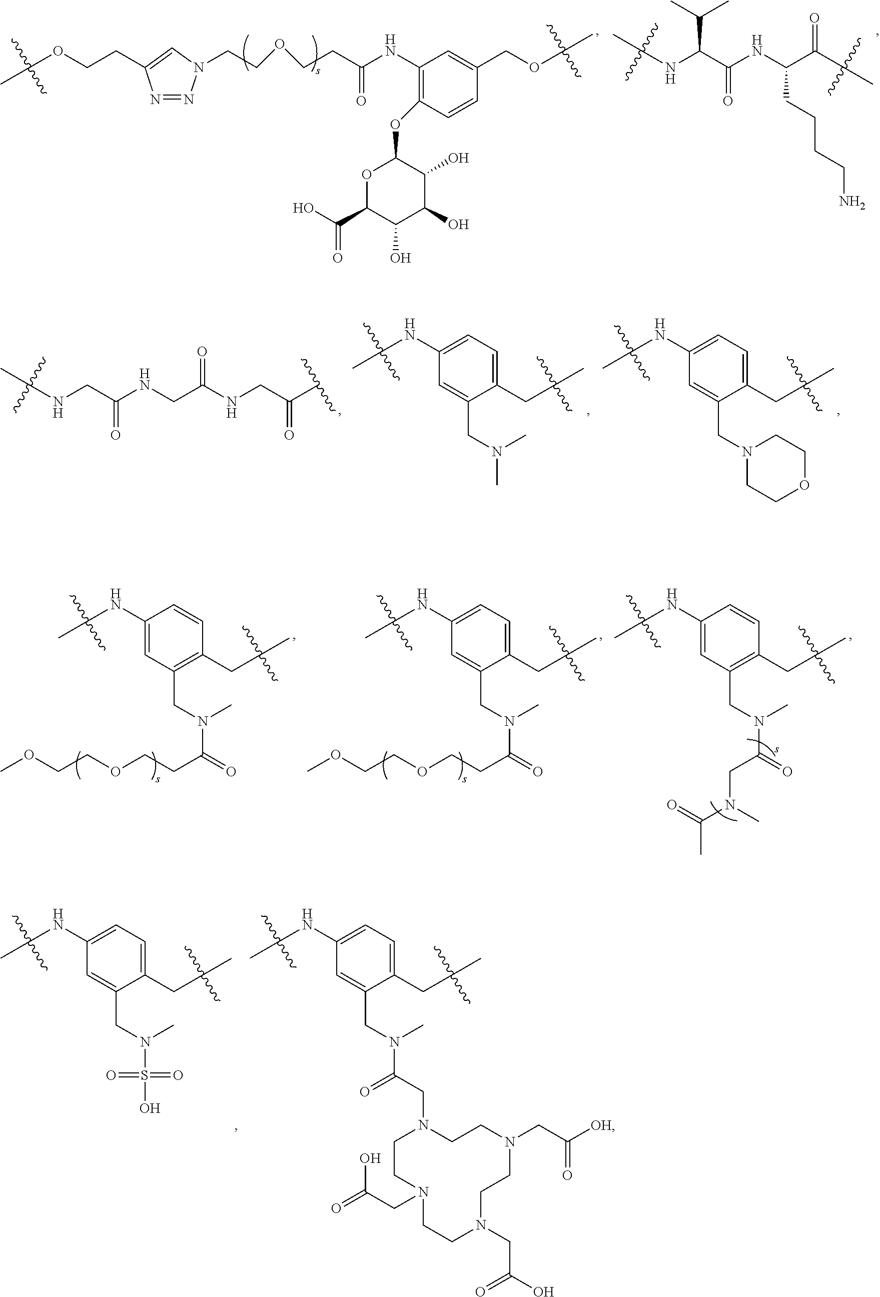

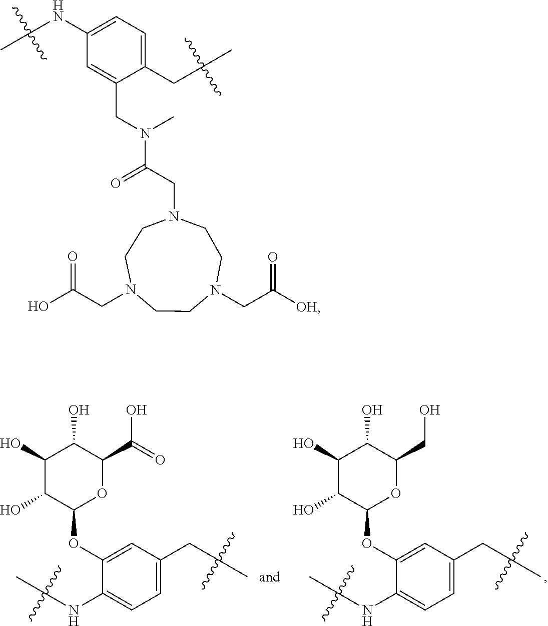

wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15.

In some embodiments, L is selected from the group consisting of the substituted or unsubstituted structural fragments composed of one or more of the following groups:

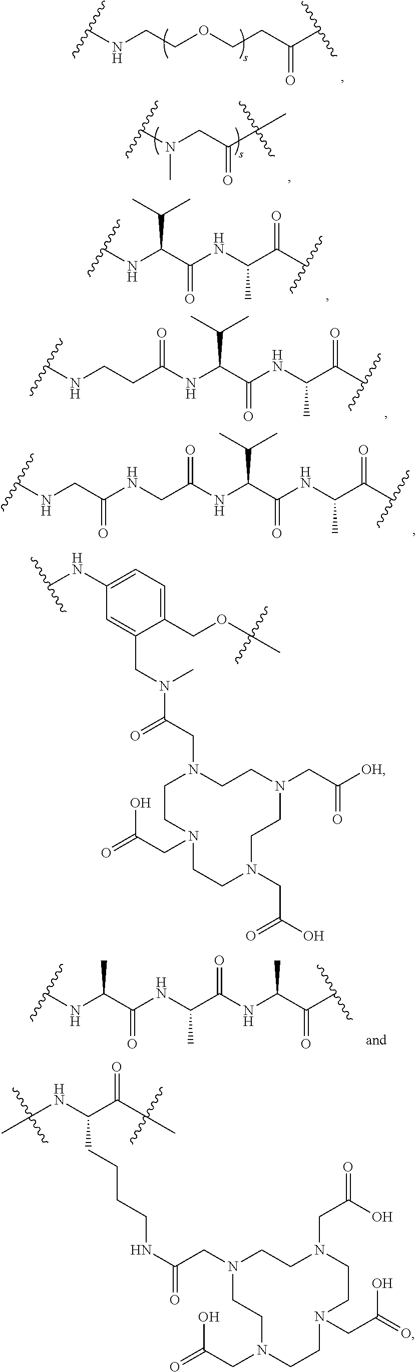

wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably, s is selected from 5, 8, 10.

In some embodiments, L is selected from the group consisting of the substituted or unsubstituted structural fragments composed of one or more of the following groups:

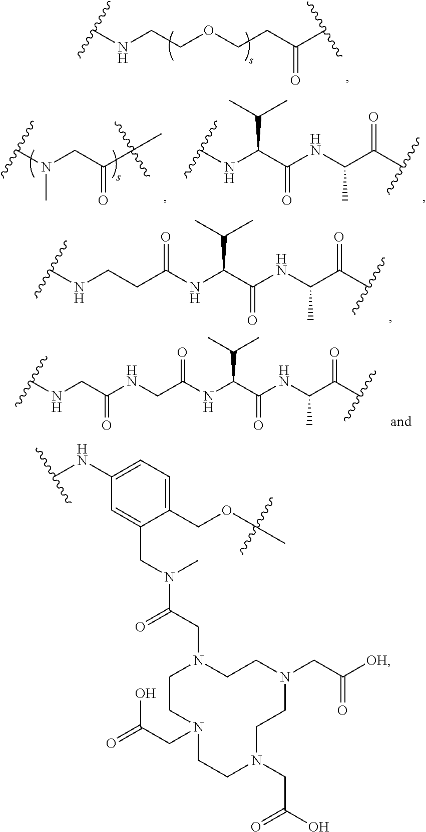

wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably, s is selected from 5, 8, 10.

In some embodiments, L is selected from the group consisting of the substituted or unsubstituted structural fragments composed of one or more of the following groups:

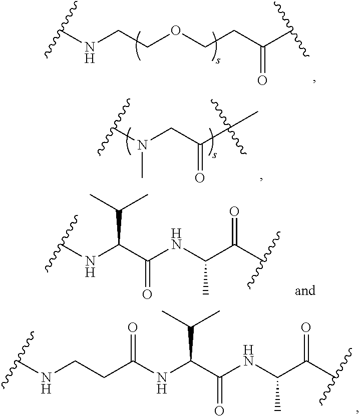

wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably, s is selected from 5, 8, 10.

In some embodiments, L comprises the following structural fragment

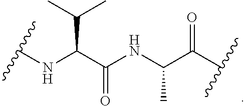 [SMILES: *N[C@H](C(=O)N[C@@H](C)C(*)=O)C(C)C]

In some embodiments, L is formed by connecting the structural fragment of Group I to one or more substituted or unsubstituted structural fragments selected from the Group II.

Group I is

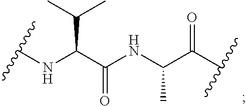 [SMILES: *N[C@H](C(=O)N[C@@H](C)C(*)=O)C(C)C]

Group II is composed of

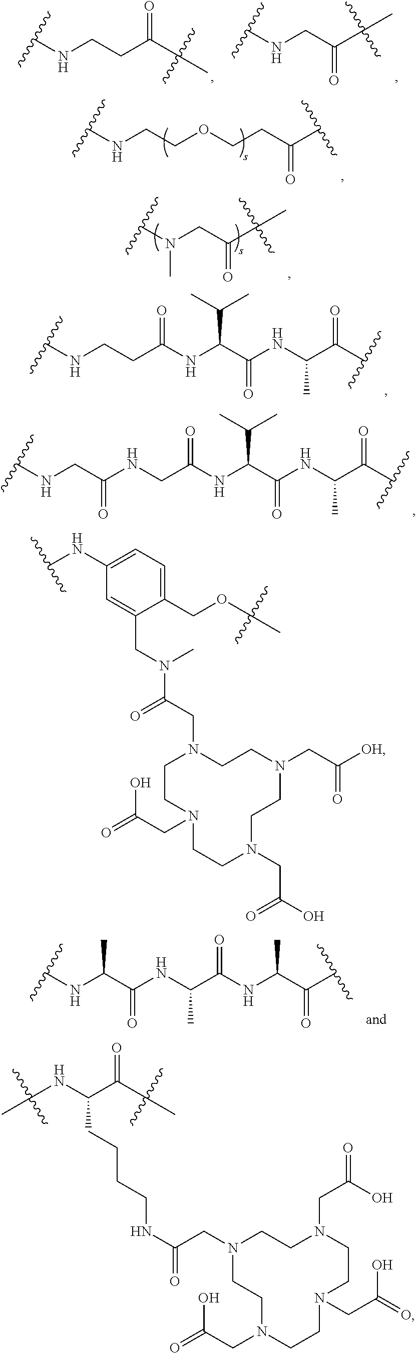

wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably s is selected from 5, 8, 10.

In some embodiments, group II is composed of

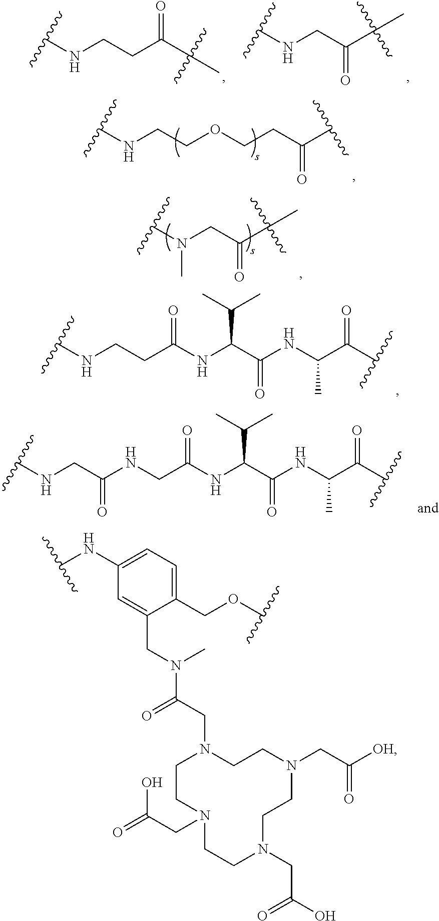

wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably s is selected from 5, 8, 10.

In some embodiments

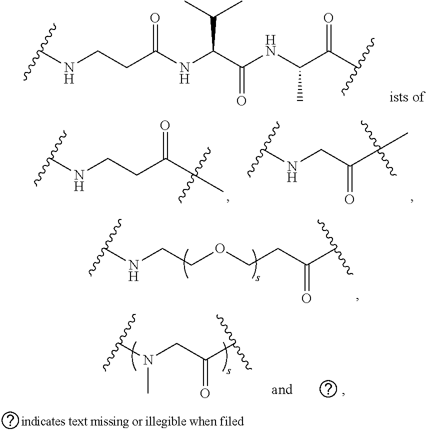

wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably s is selected from 5, 8, 10.

In some embodiments, E is a single bond, substituted or unsubstituted —NH—CH₂—, or selected from the group consisting of the following substituted or unsubstituted structural fragments:

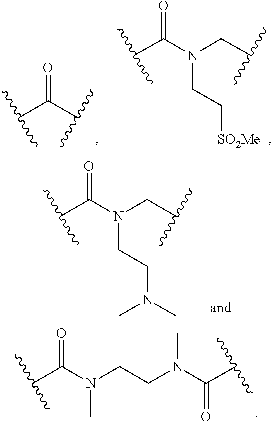

In some embodiments, E is a single bond, substituted or unsubstituted —NH—CH₂— or

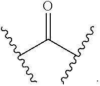 [SMILES: *C(*)=O]

In some embodiments, E is substituted or unsubstituted —NH—CH₂— or

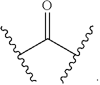 [SMILES: *C(*)=O]

In some embodiments,

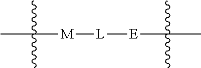

is selected from the group consisting of the following substituted or unsubstituted structures:

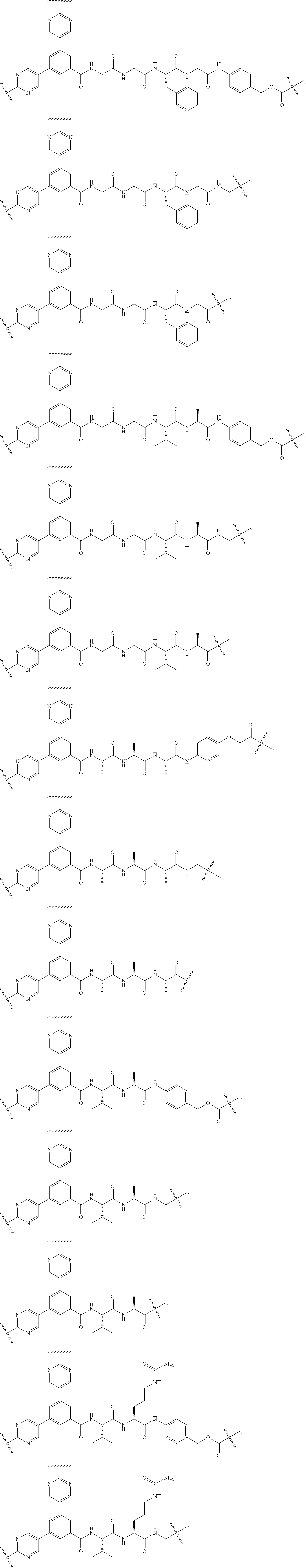

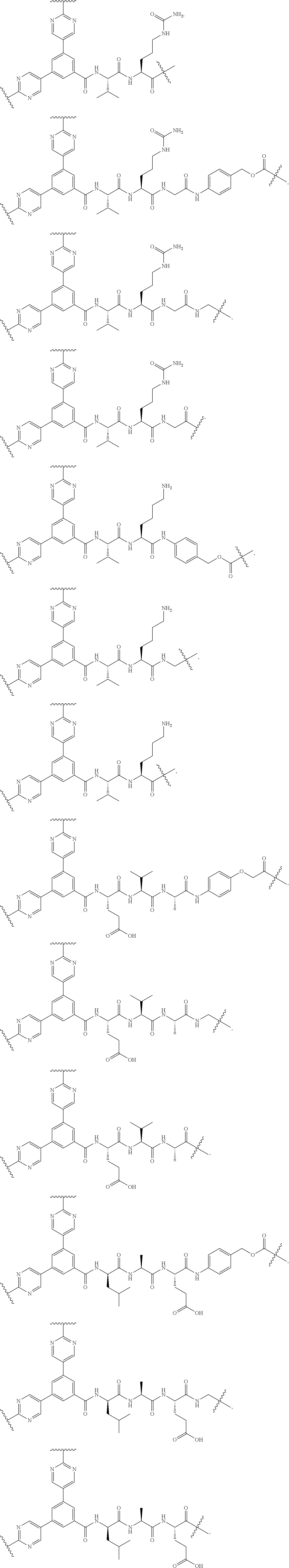

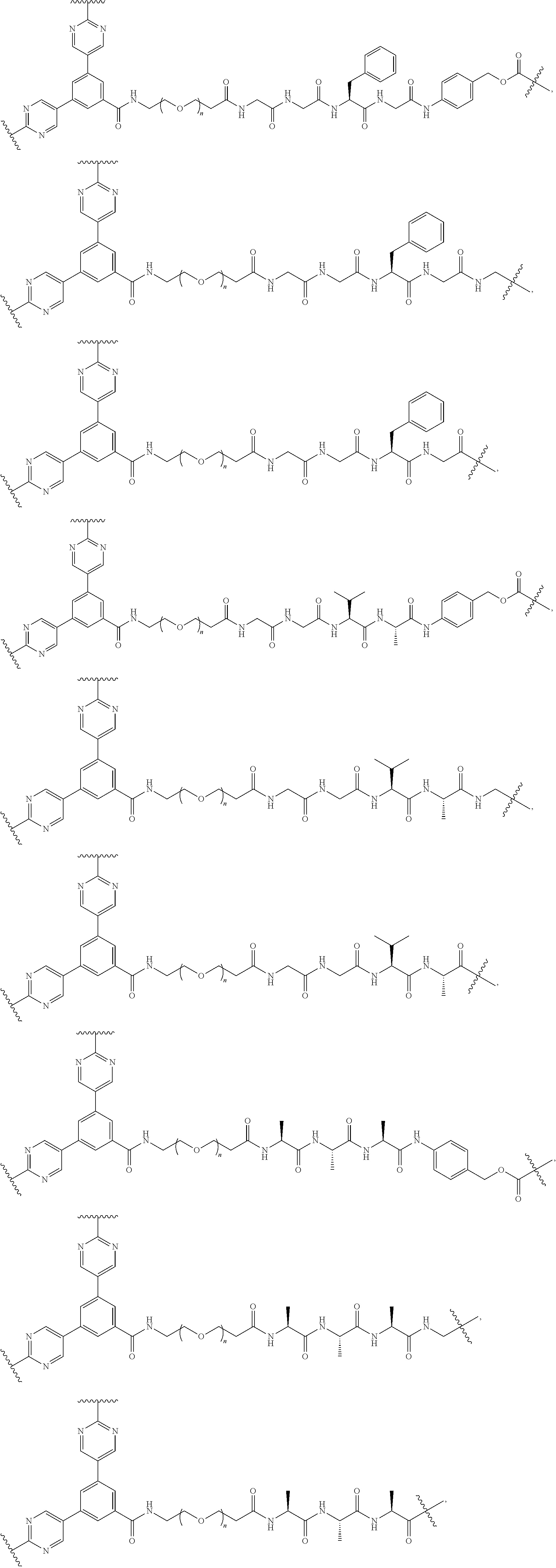

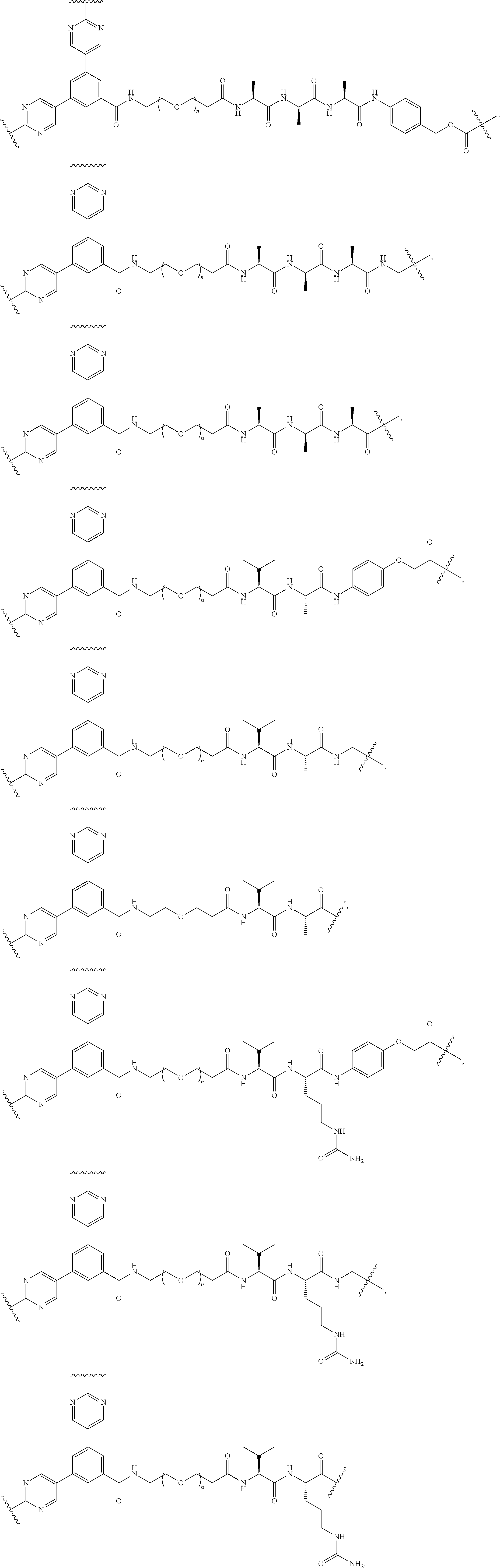

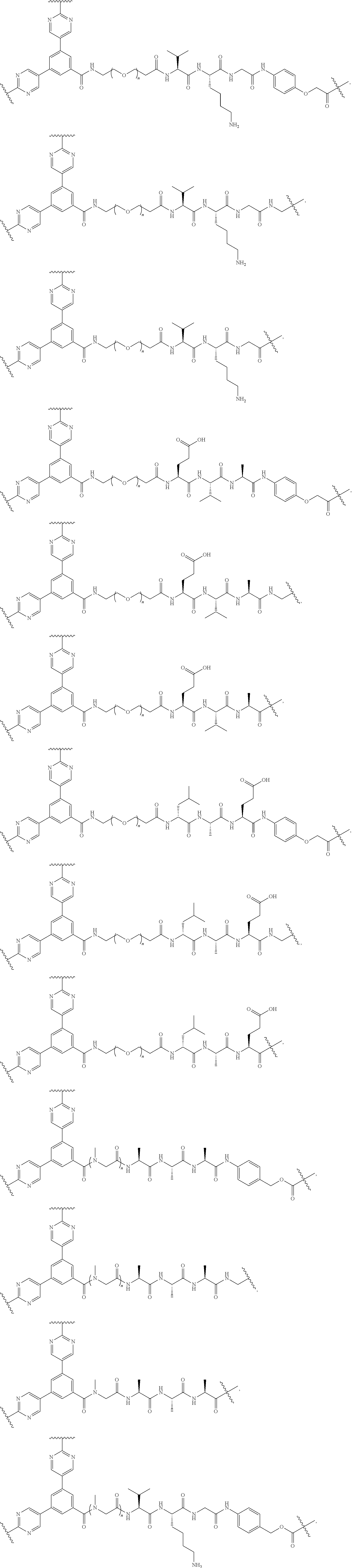

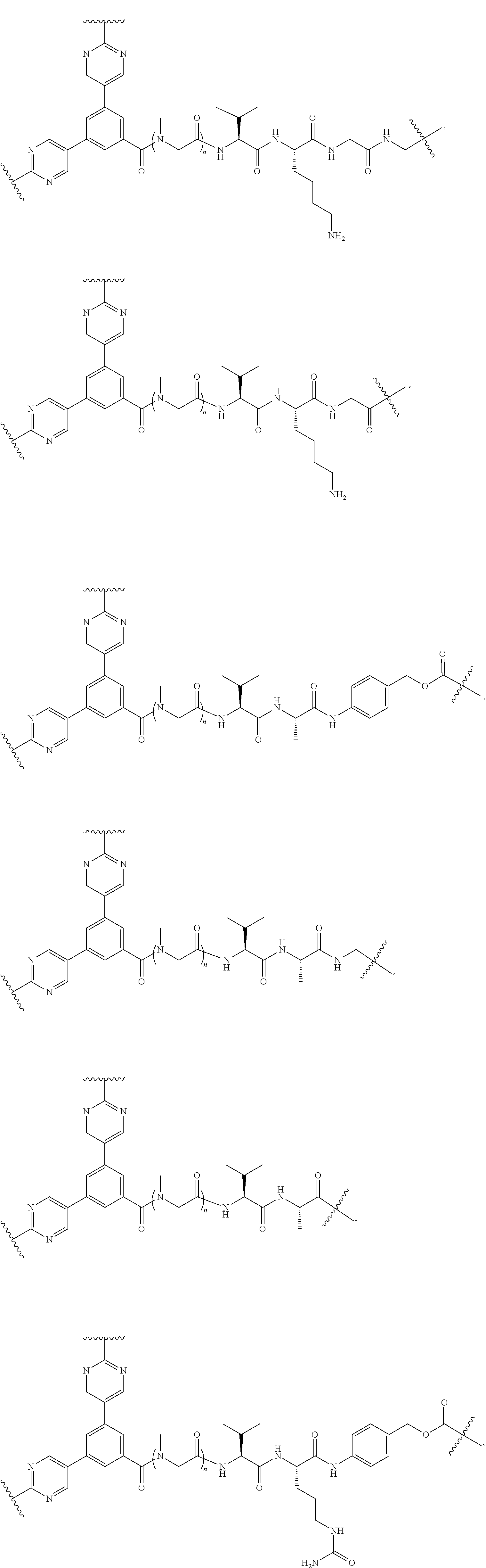

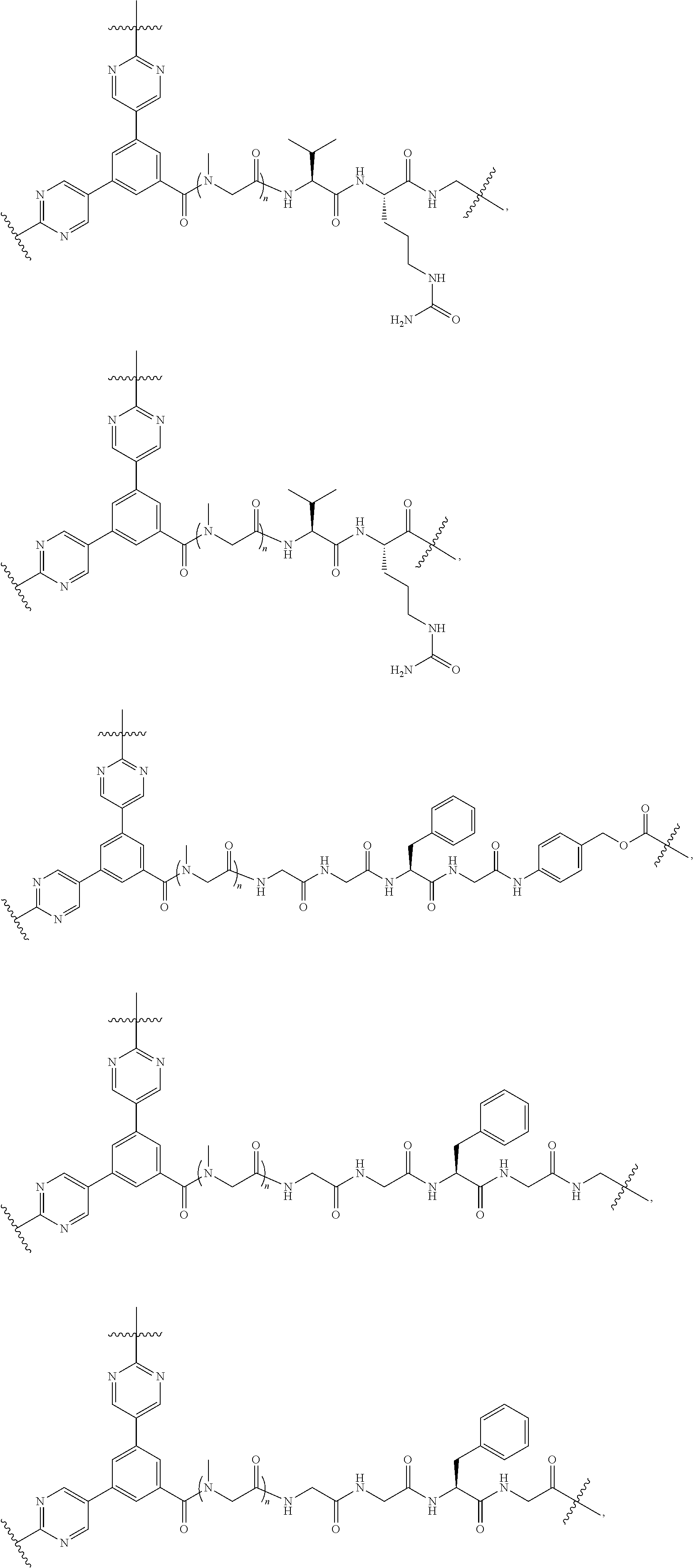

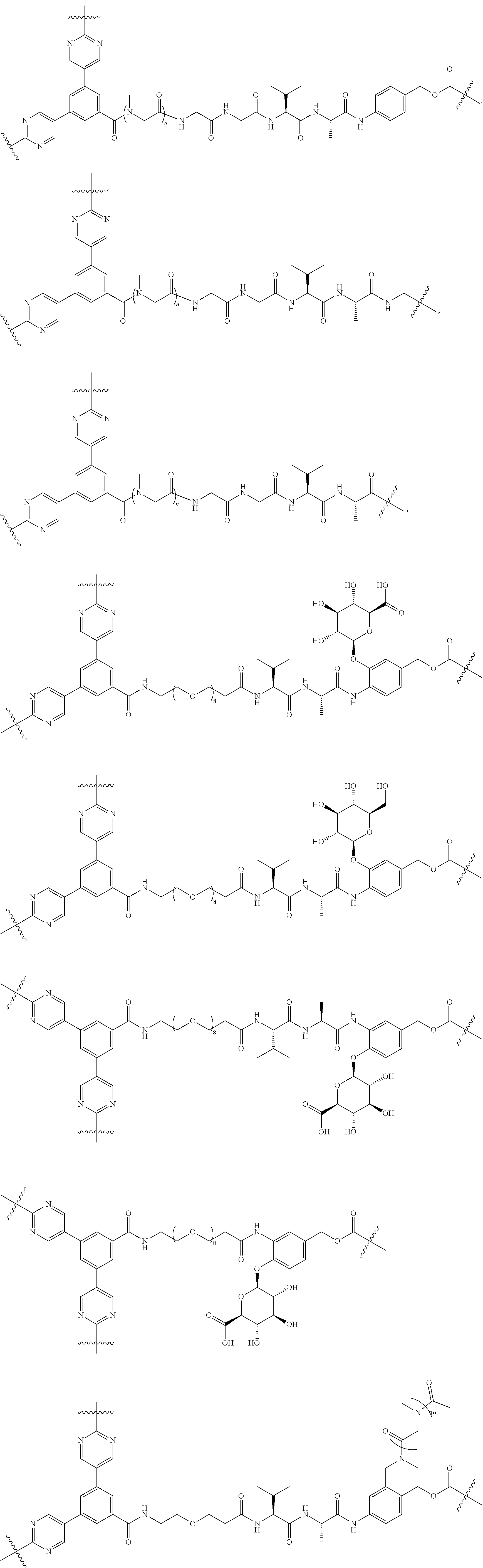

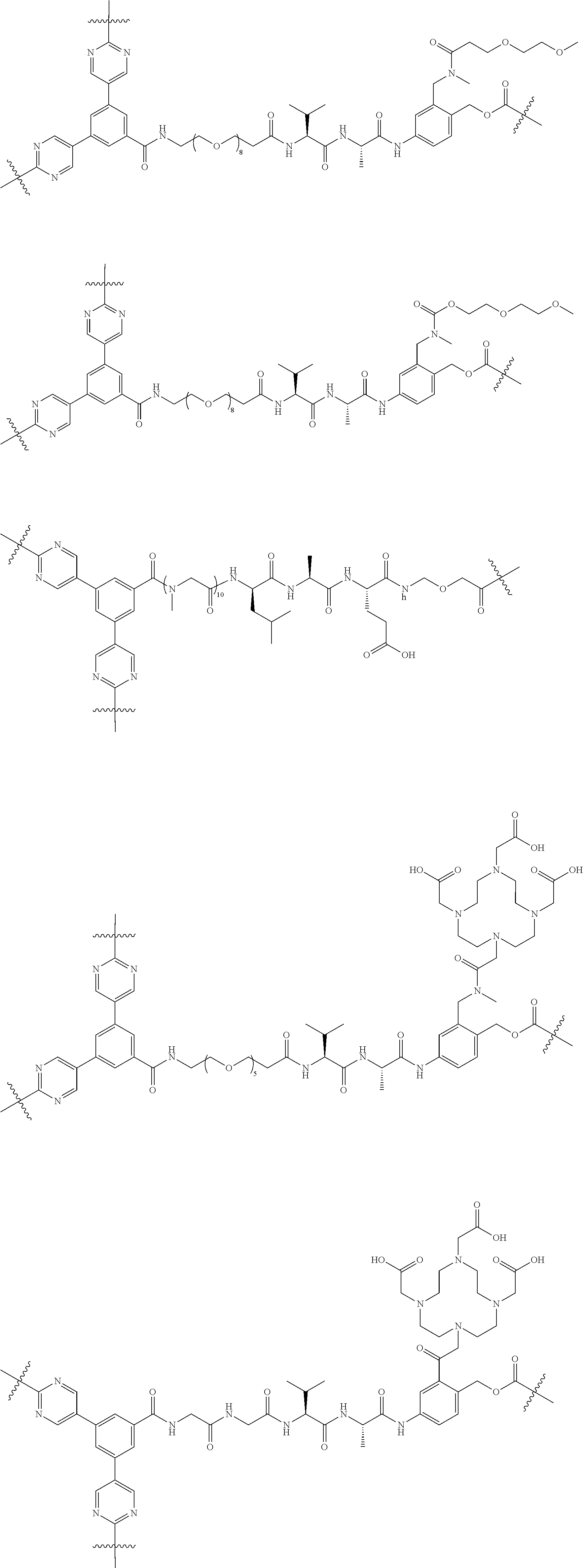

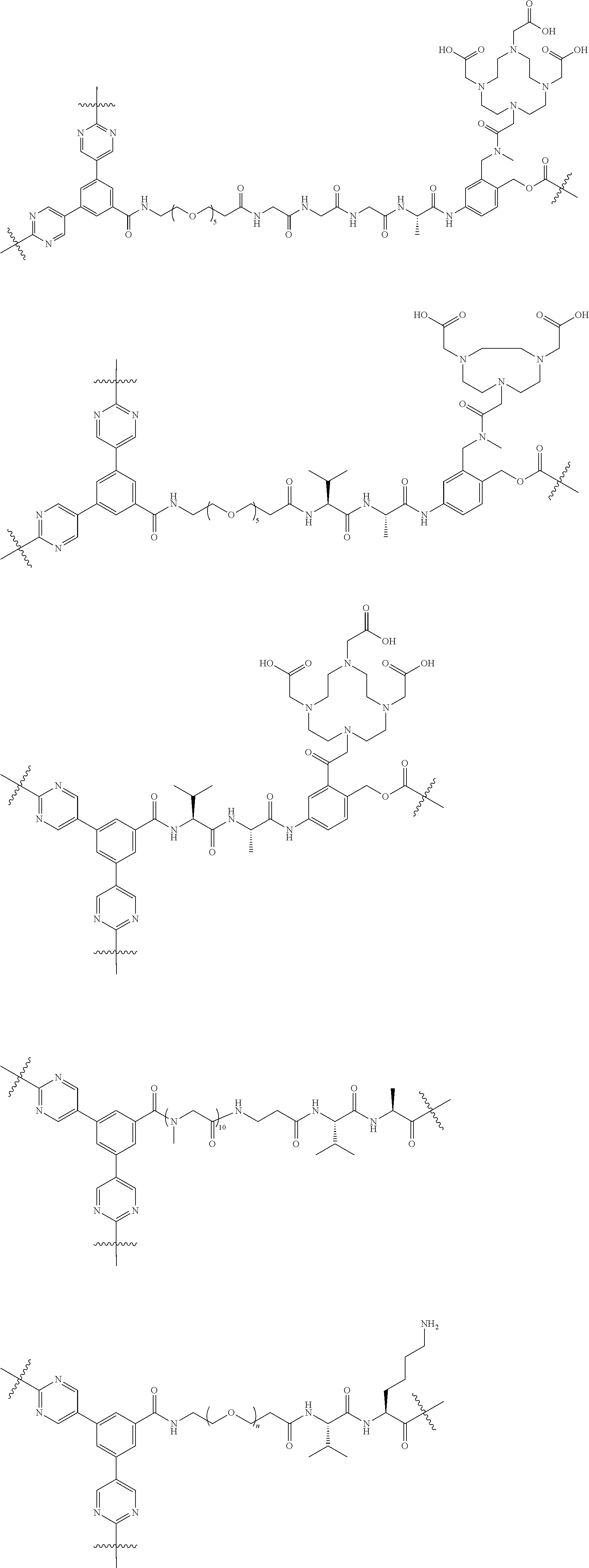

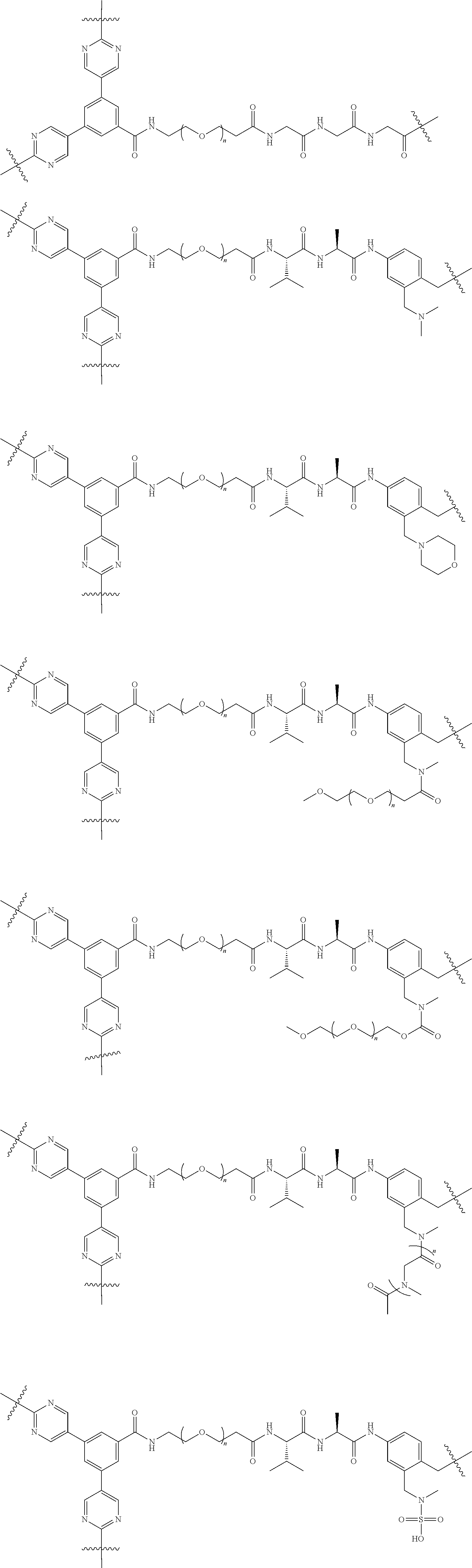

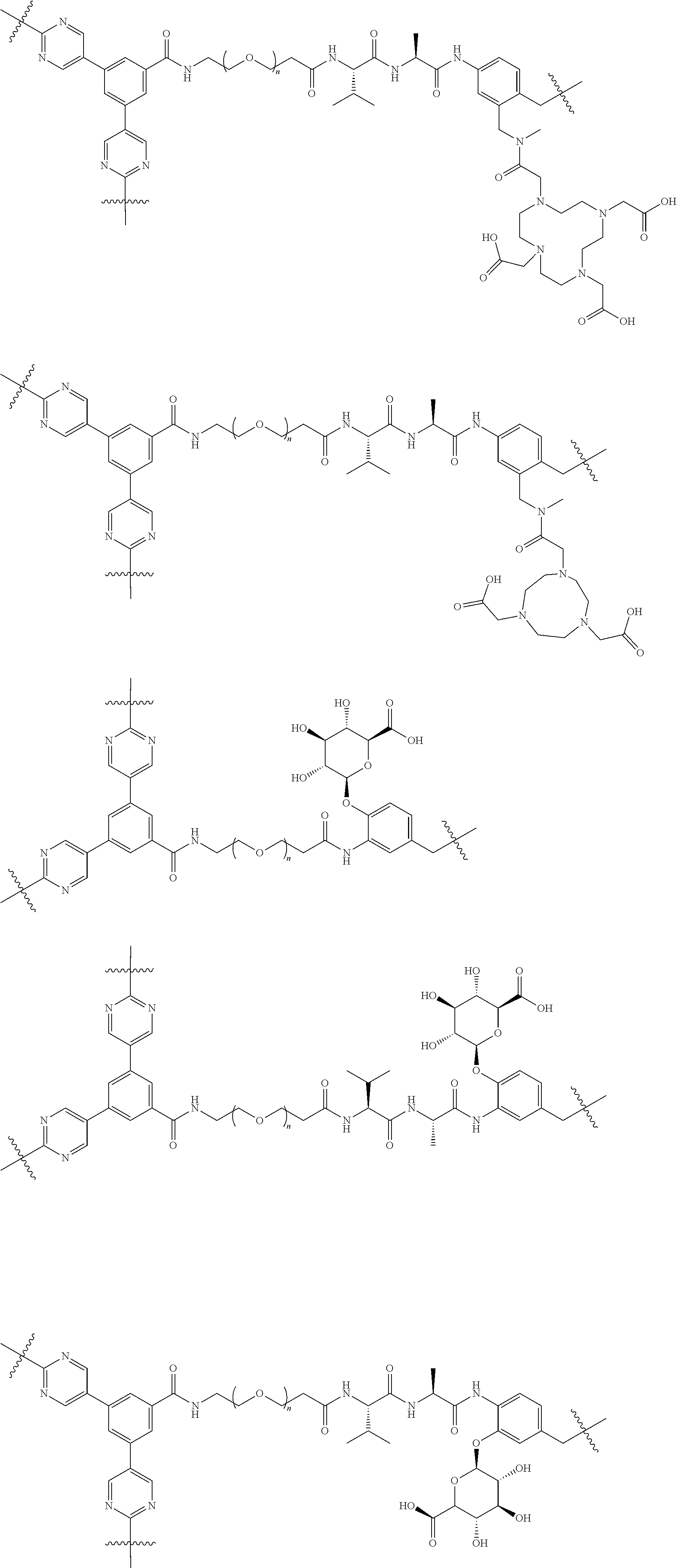

n is an integer selected from 1 to 20. In some embodiments, n is an integer selected from 1 to 15, such as an integer of 1 to 12, 3 to 12, such as 1, 2, 3, 4, 5, 6, 7, 8, 9, 10, 11, 12, 13, 14 or 15.

In some embodiments,

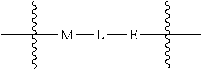

is selected from the group consisting of the following substituted or unsubstituted structures:

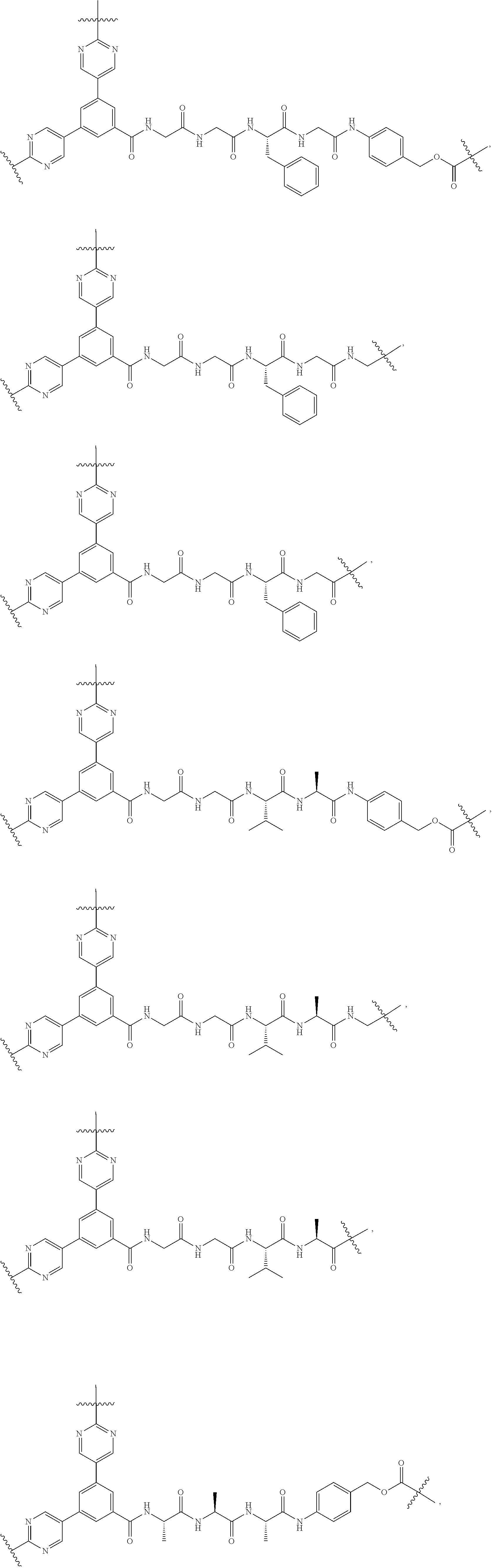

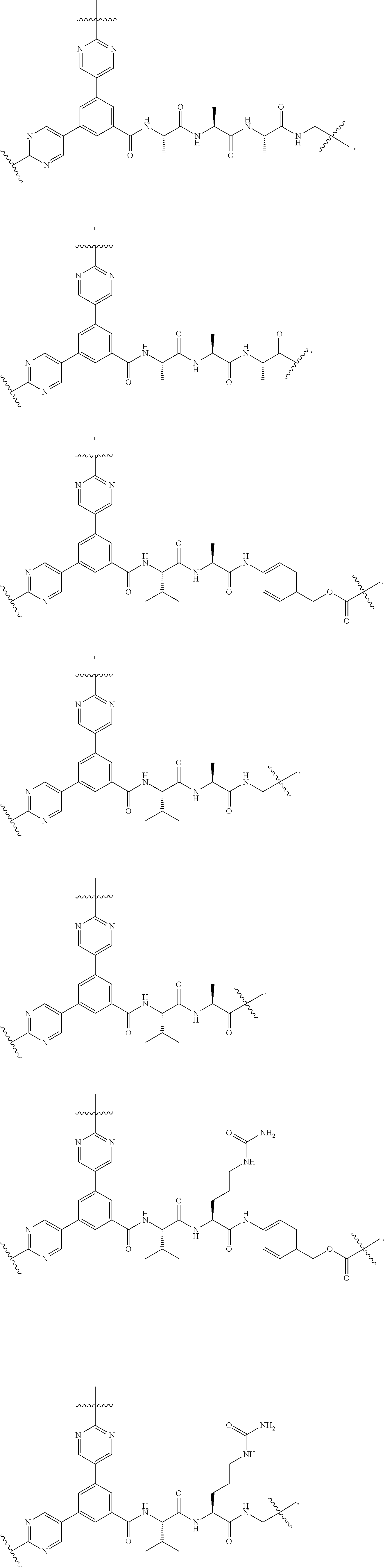

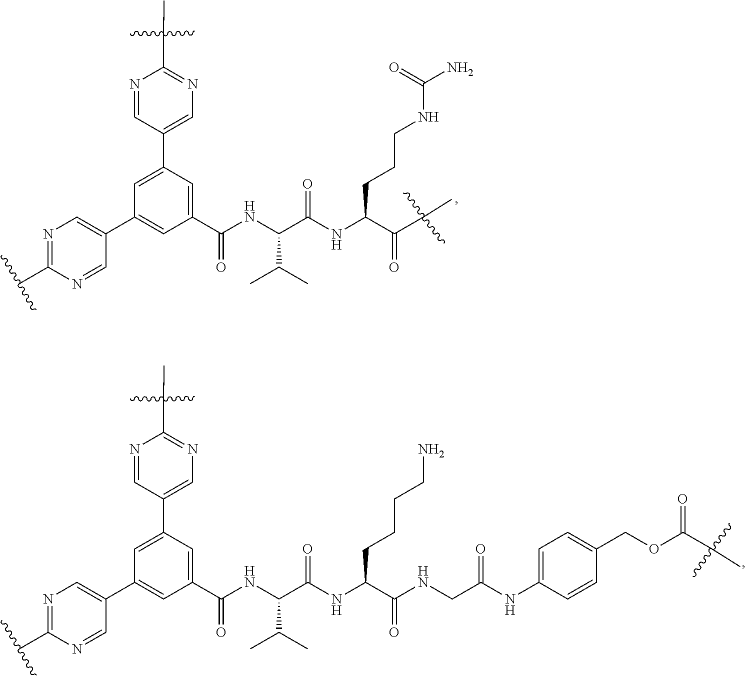 [SMILES: *C(=O)[C@H](CCCNC(N)=O)NC(=O)[C@@H](NC(=O)c1cc(-c2cnc(*)nc2)cc(-c2cnc(*)nc2)c1)C(C)C ; *C(=O)OCc1ccc(NC(=O)CNC(=O)[C@H](CCCCN)NC(=O)[C@@H](NC(=O)c2cc(-c3cnc(*)nc3)cc(-c3cnc(*)nc3)c2)C(C)C)cc1]

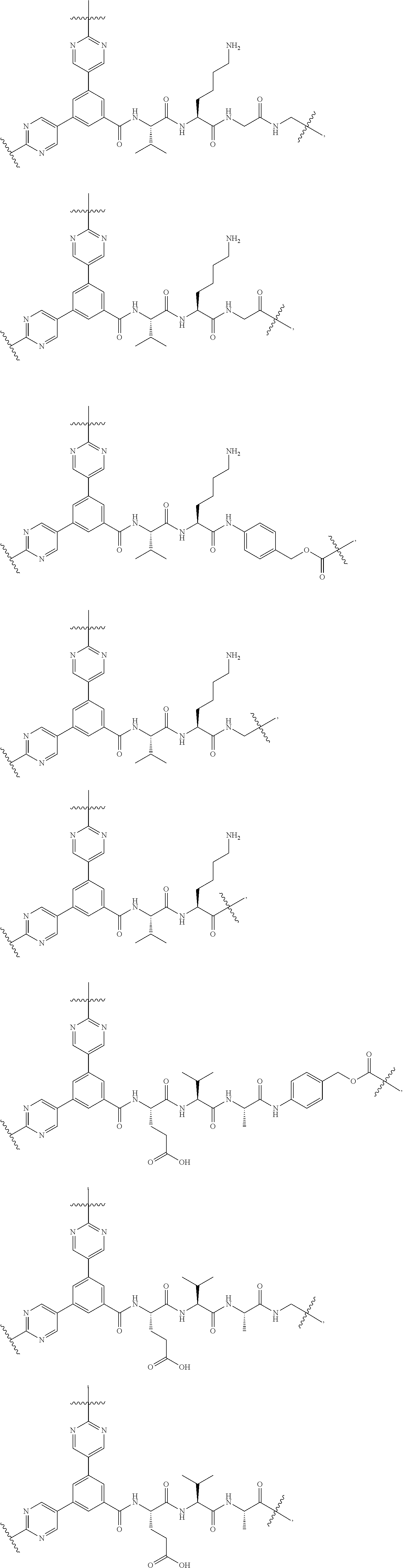

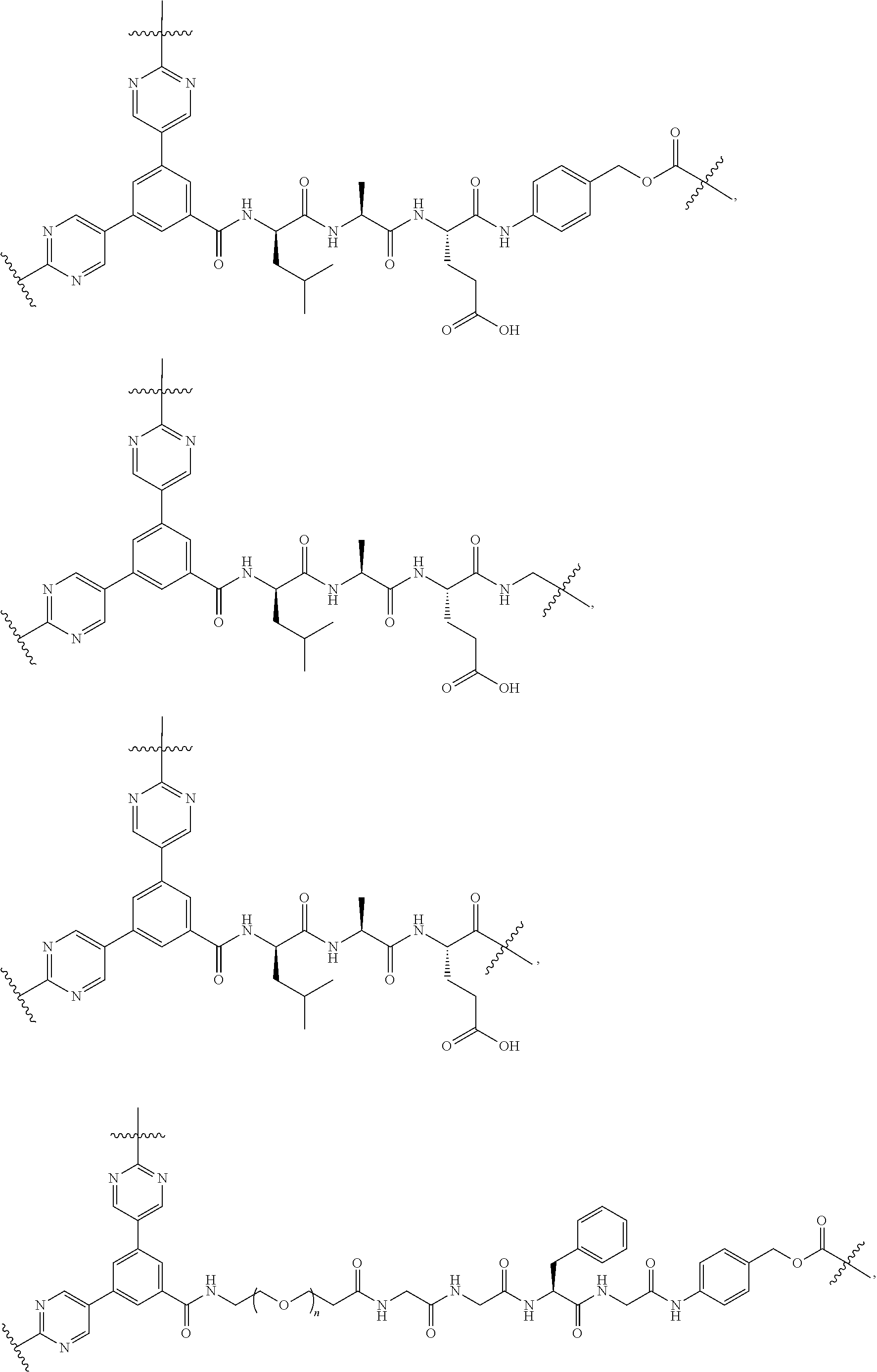

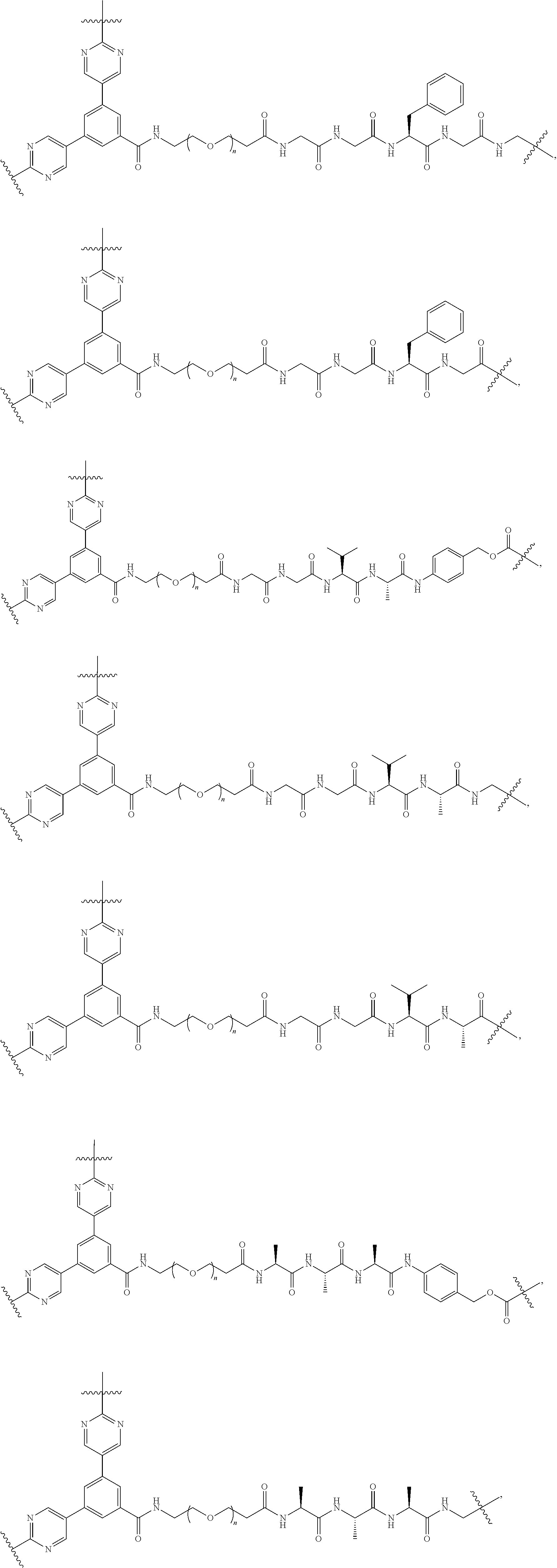

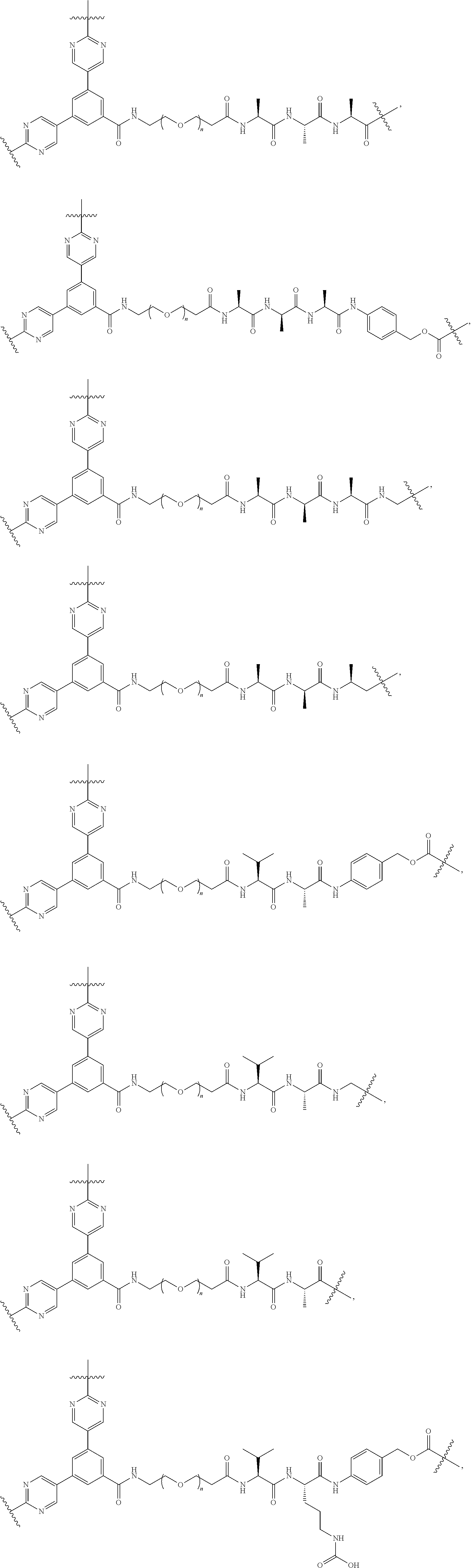

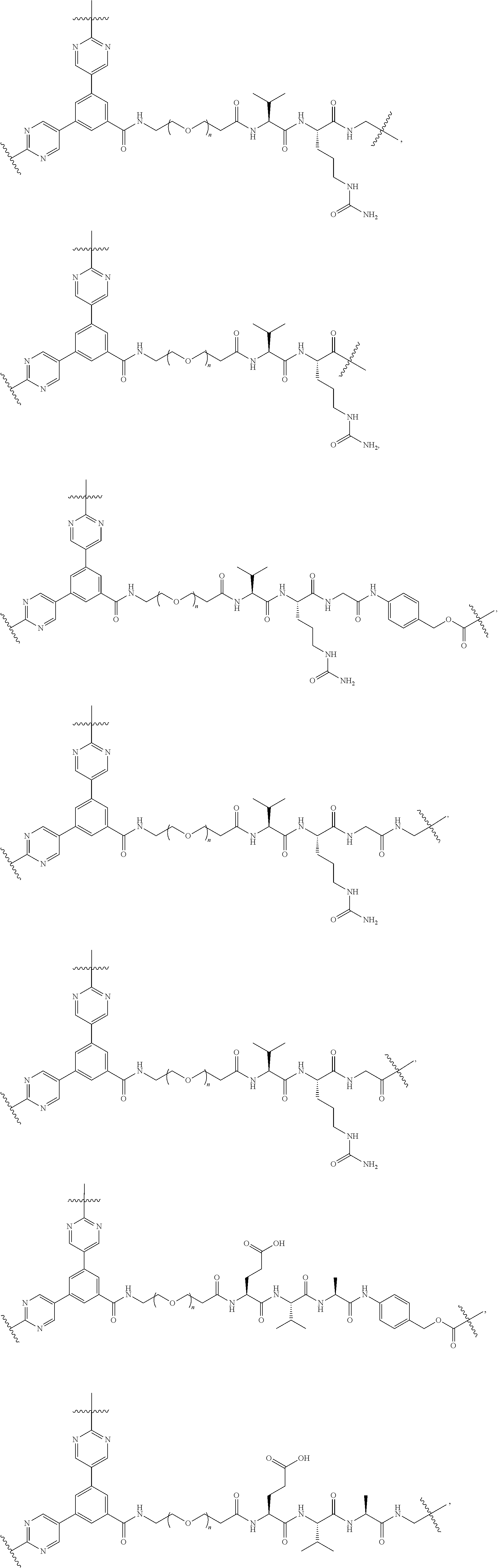

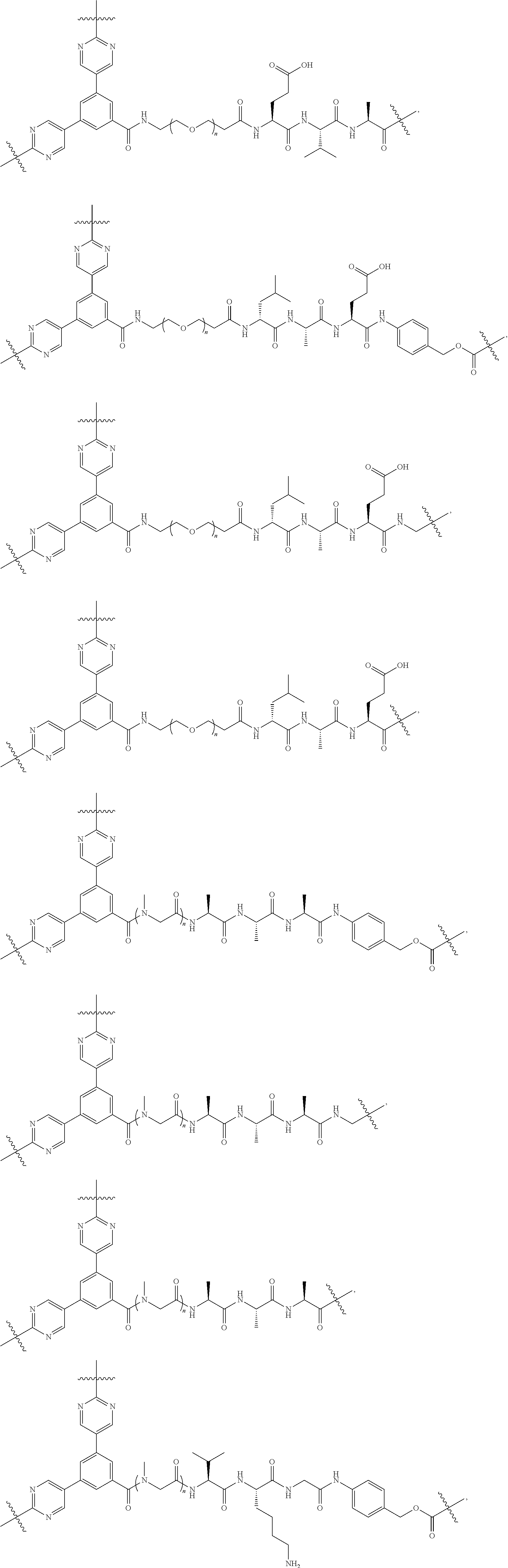

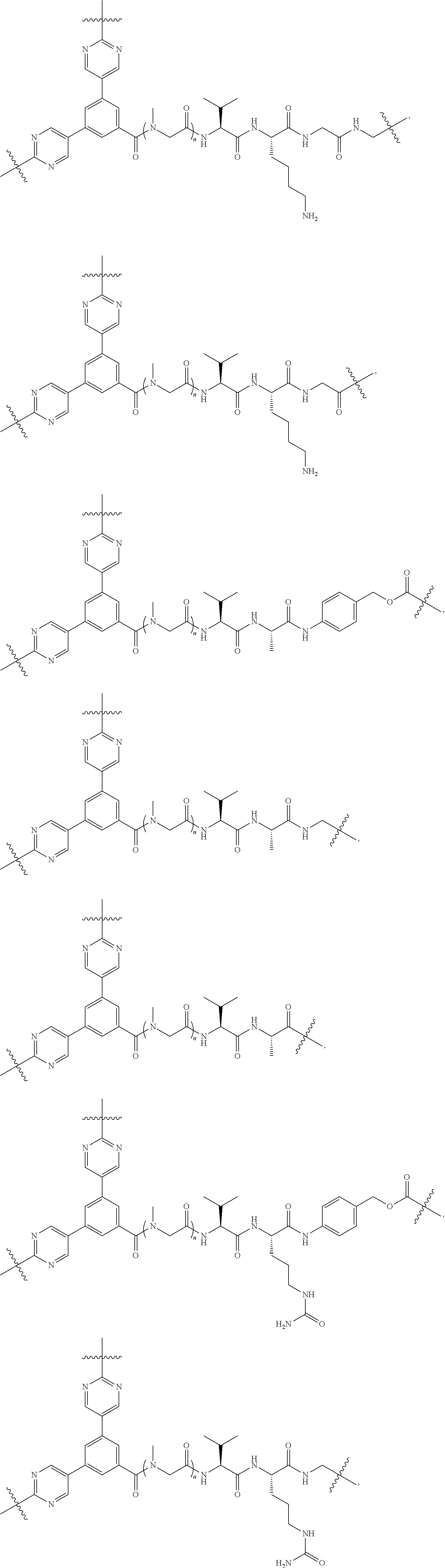

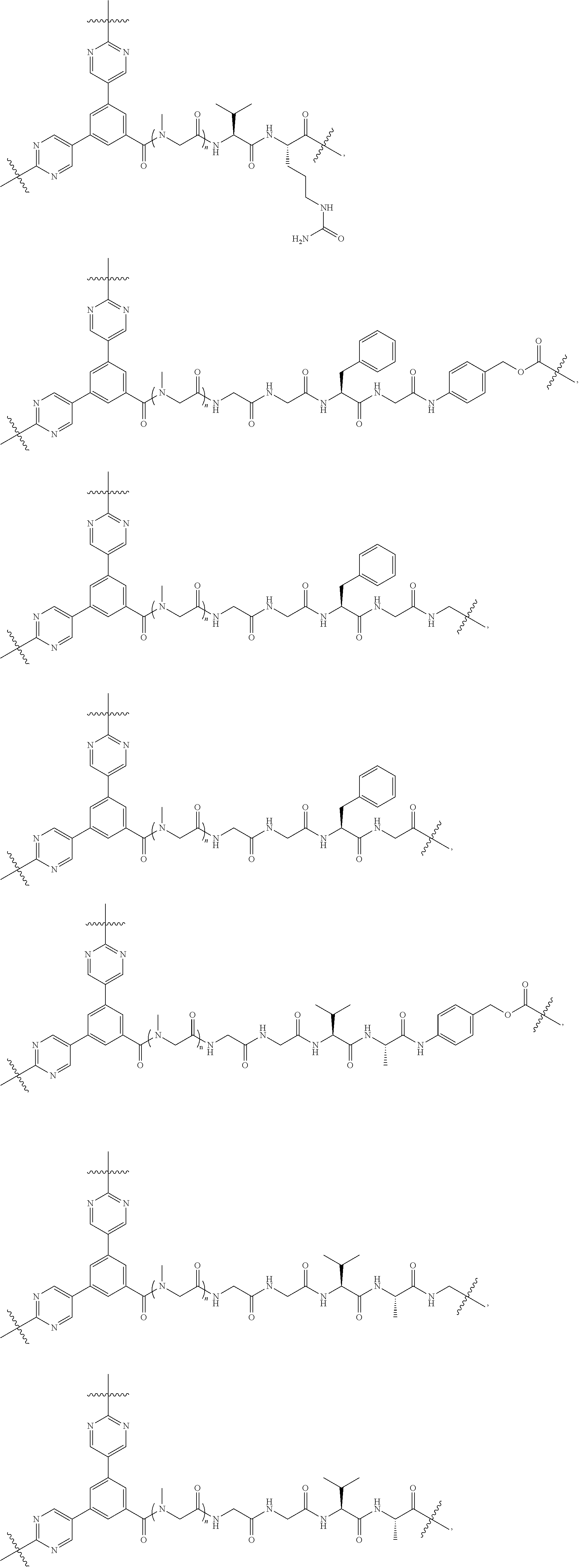

n is an integer selected from 1 to 20. In some embodiments, n is an integer selected from 1 to 15, such as an integer of 1 to 12, 3 to 12, such as 1, 2, 3, 4, 5, 6, 7, 8, 9, 10, 11, 12, 13, 14 or 15.

In some embodiments,

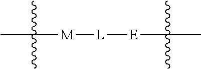

is selected from the group consisting of the following substituted or unsubstituted structures:

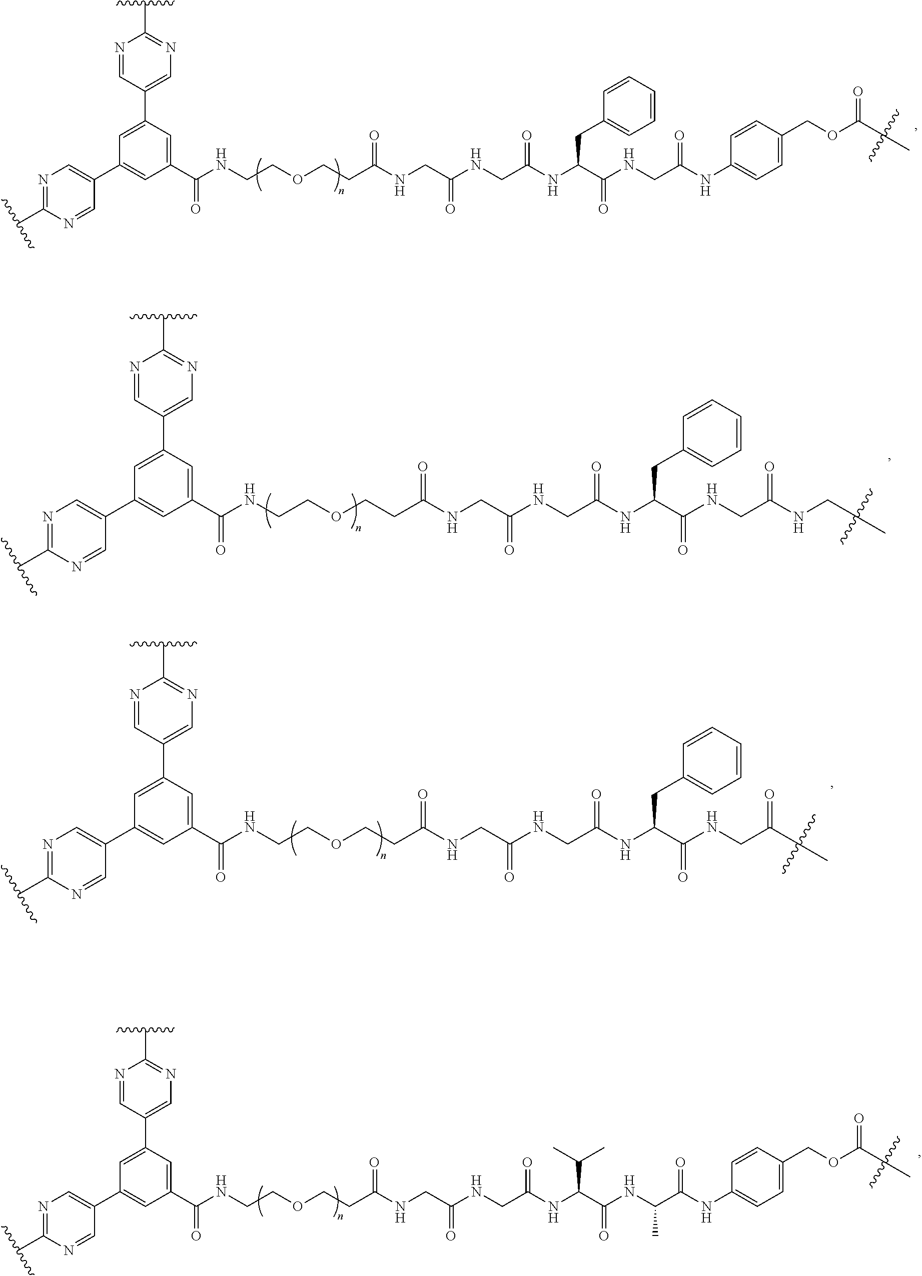

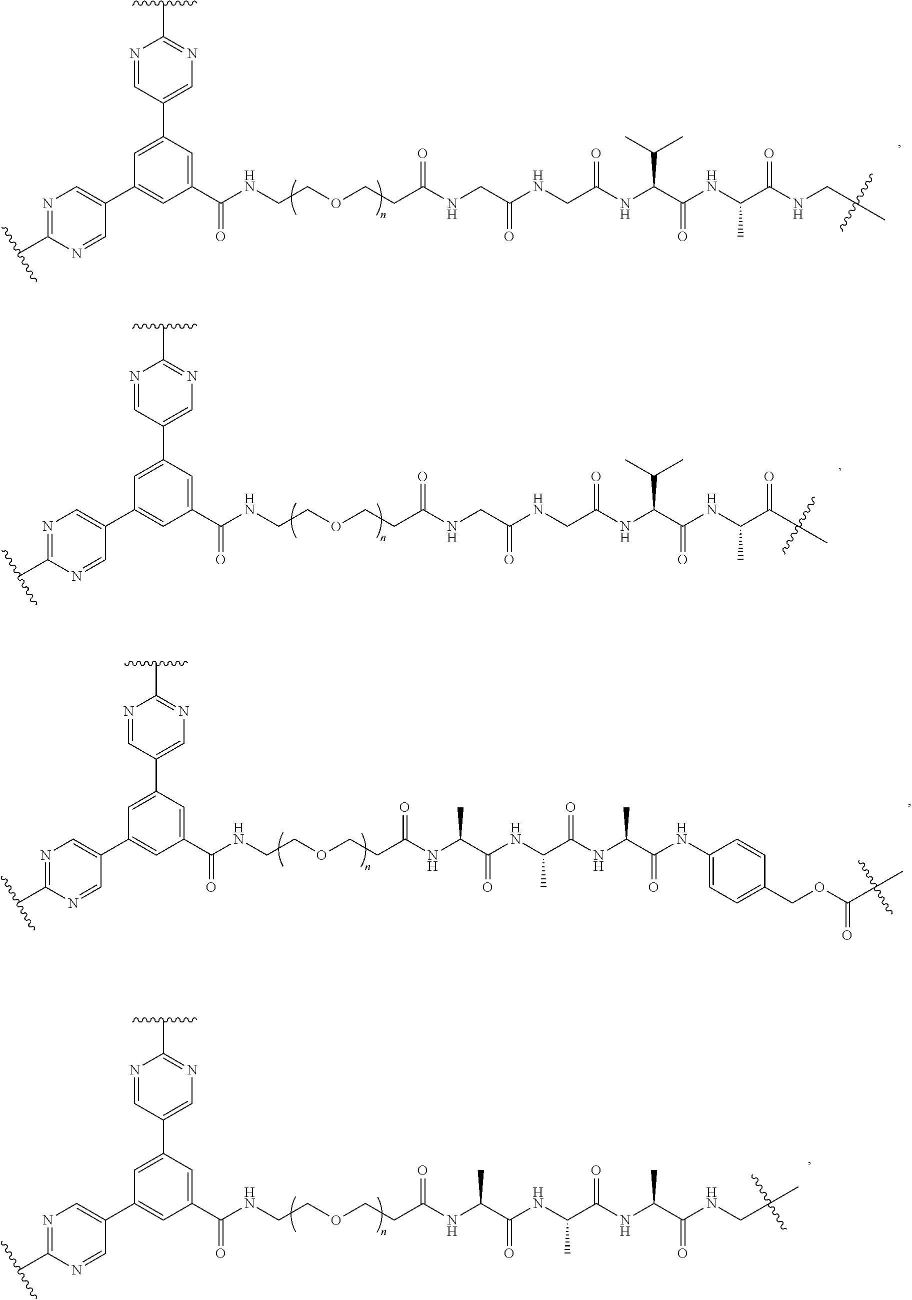

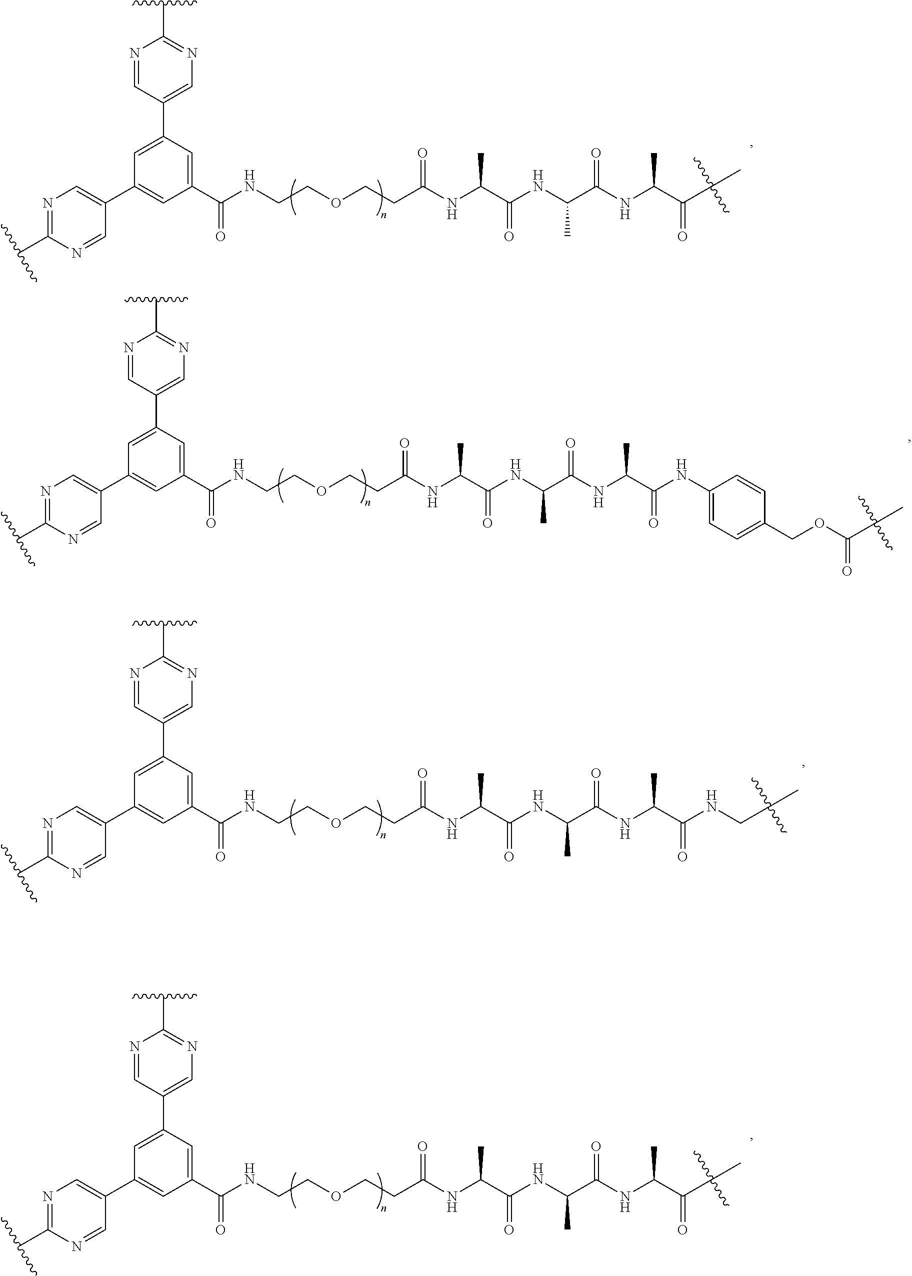

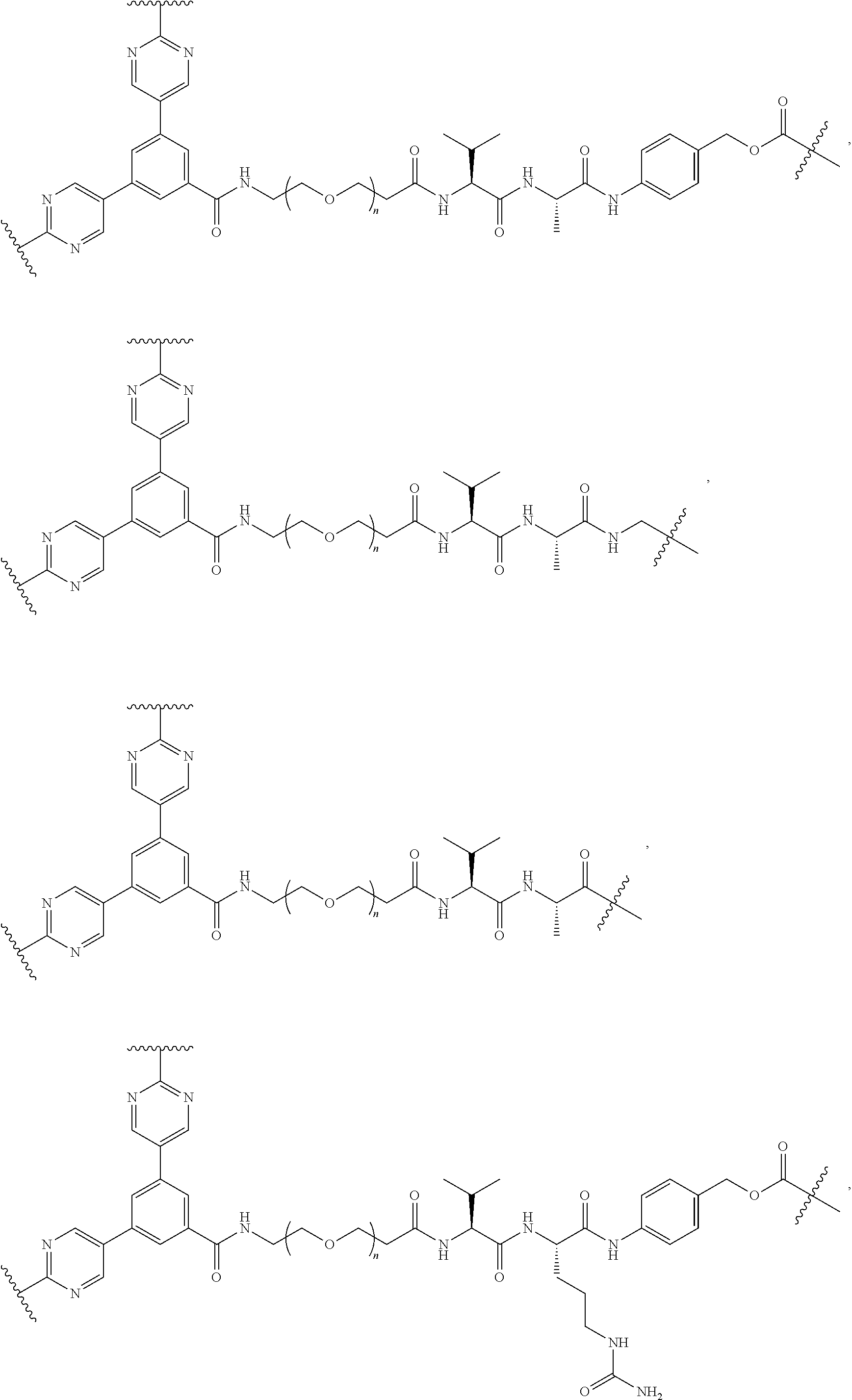

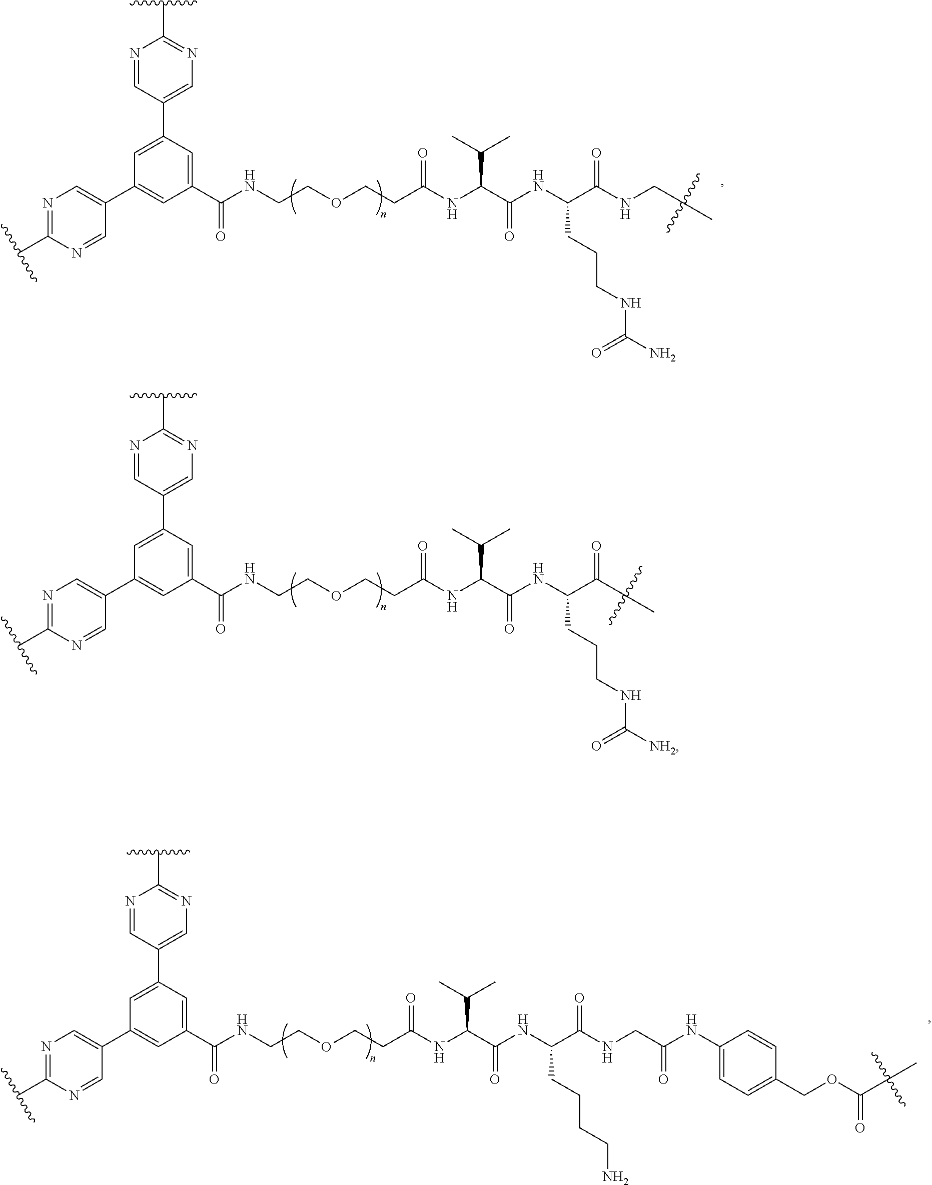

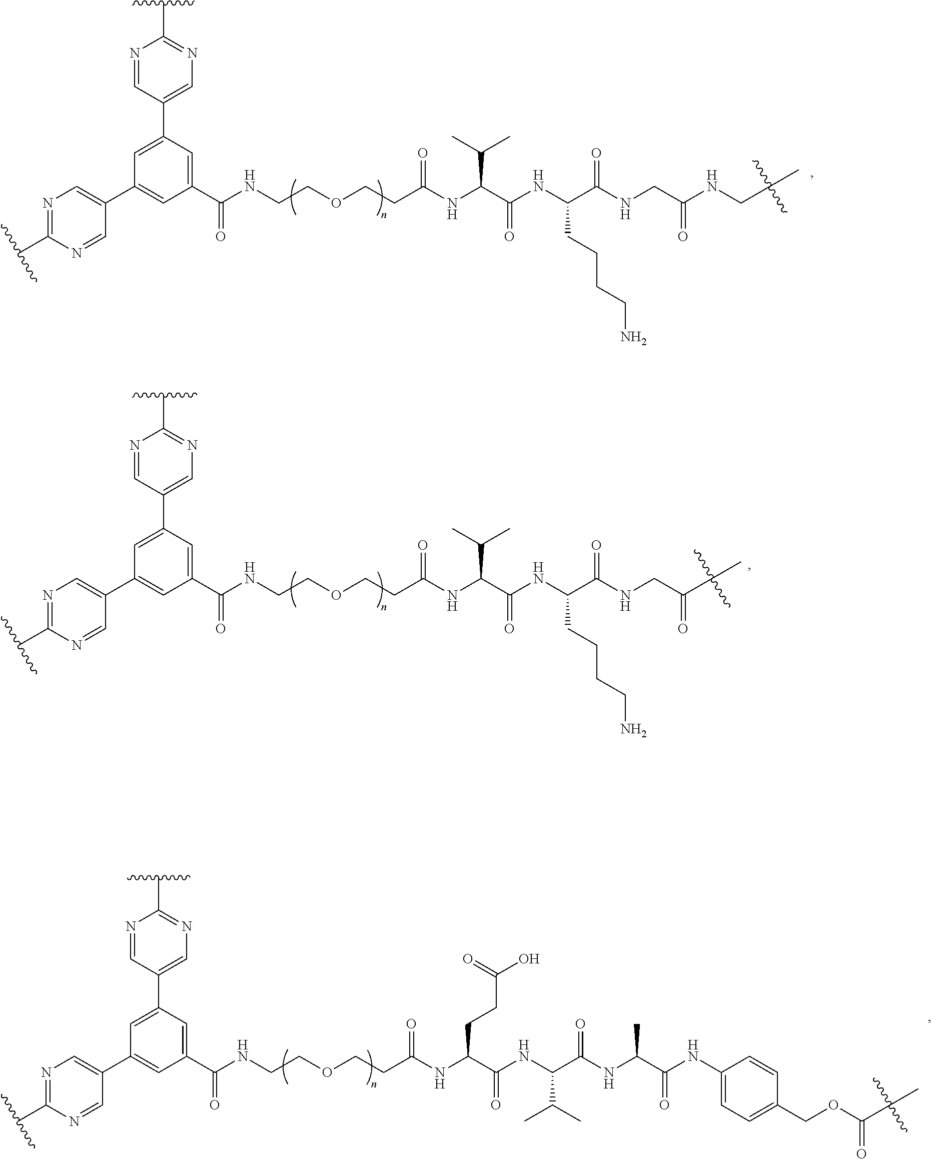

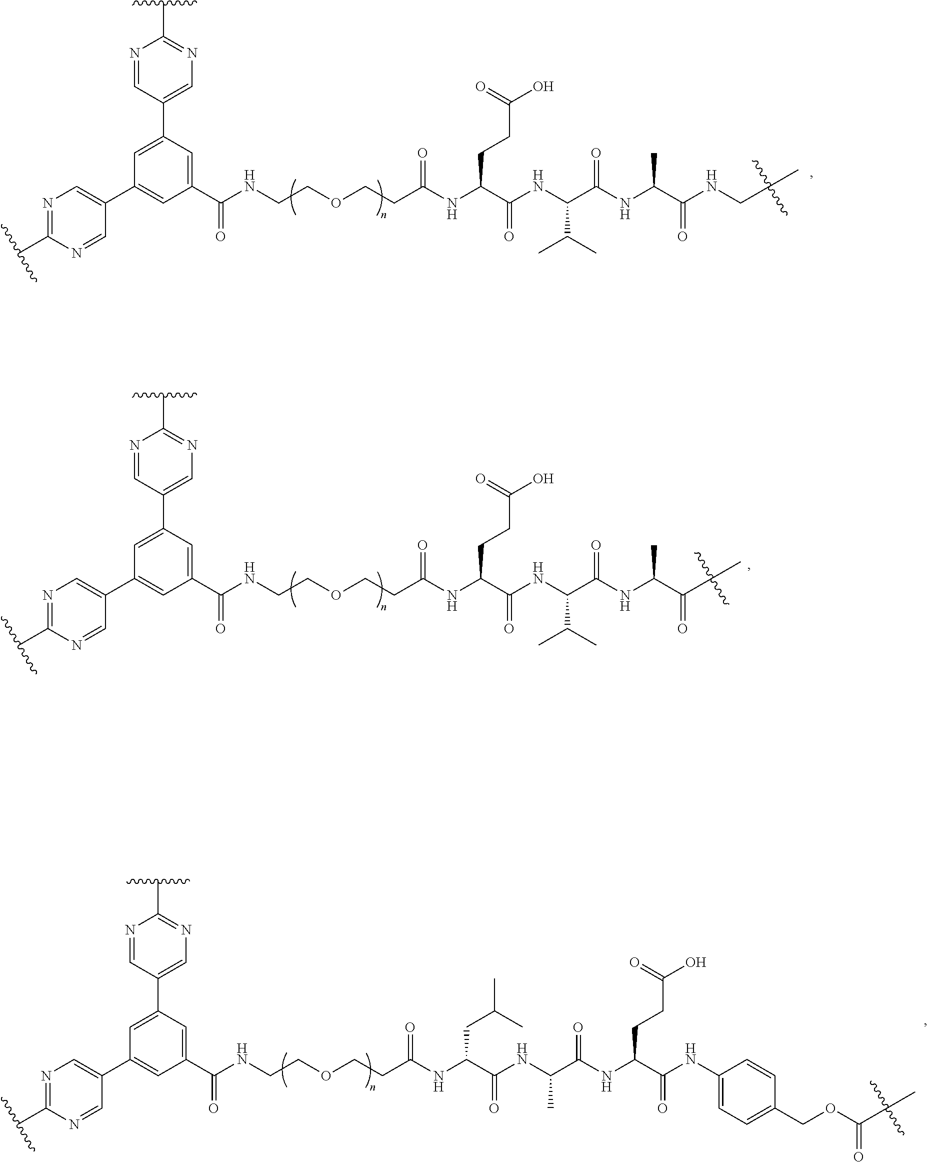

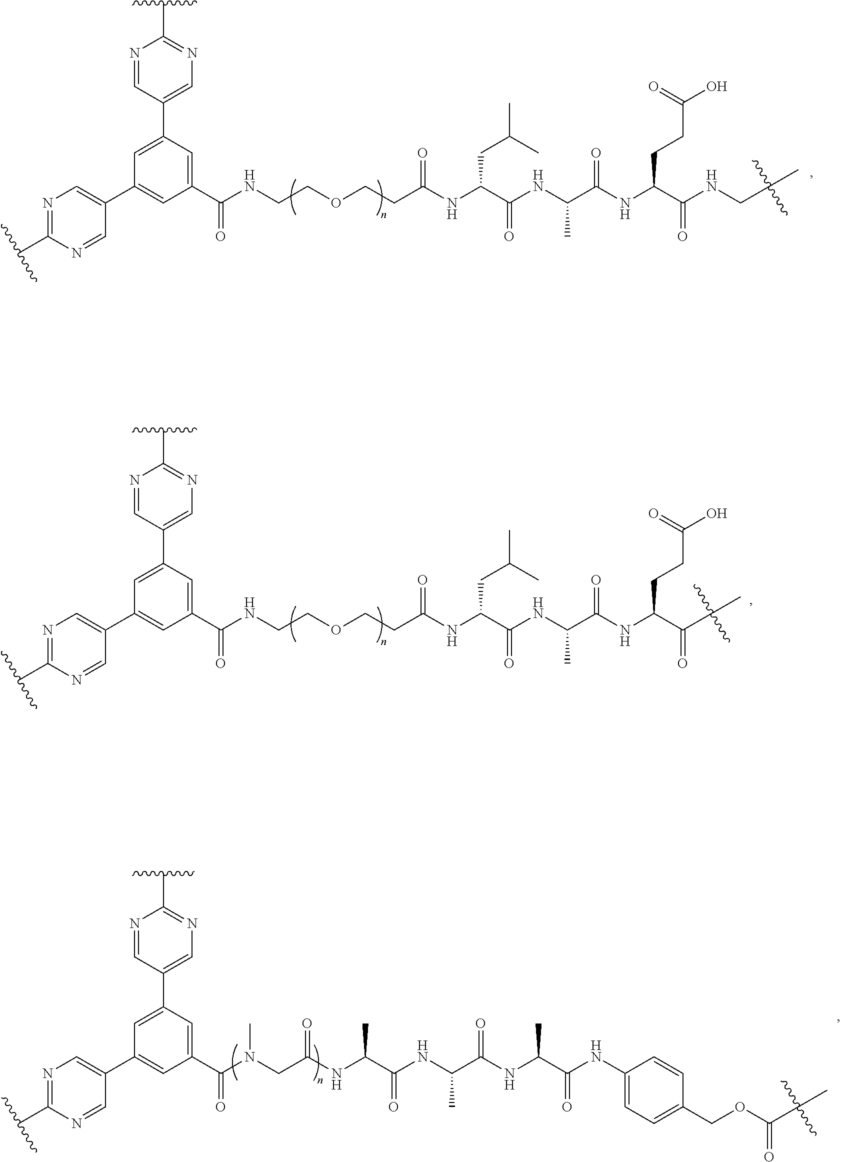

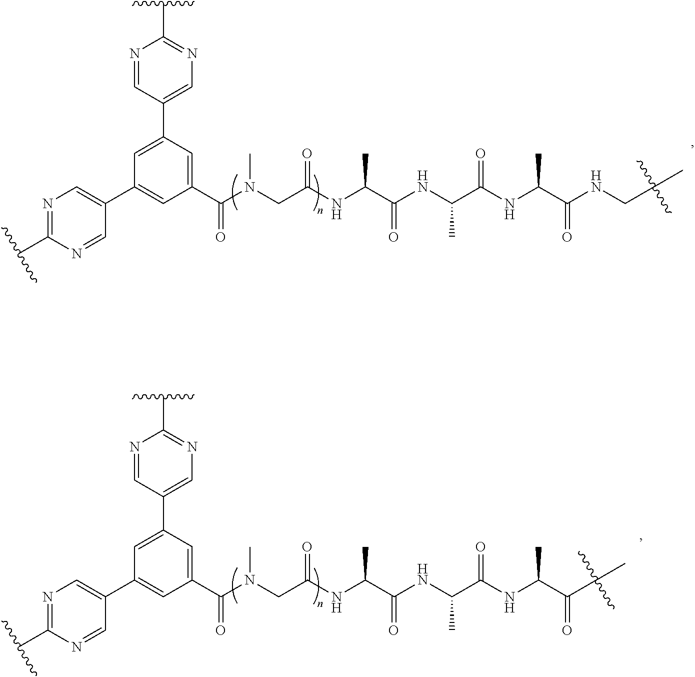

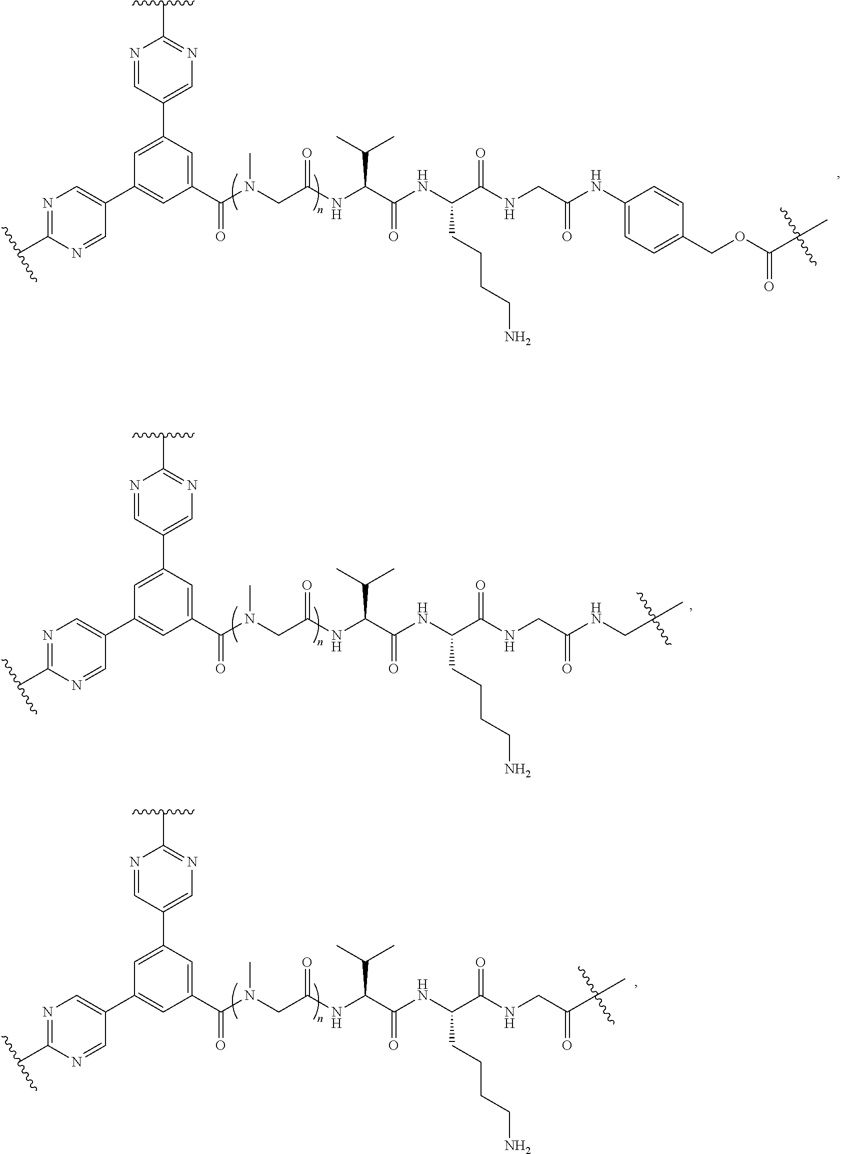

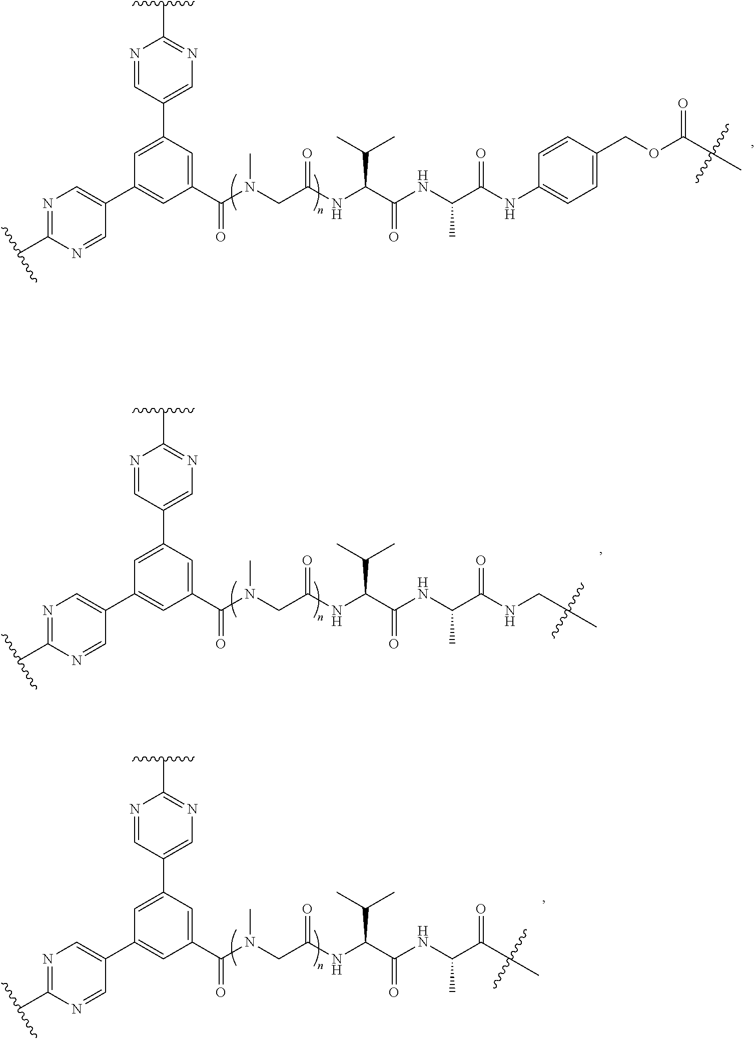

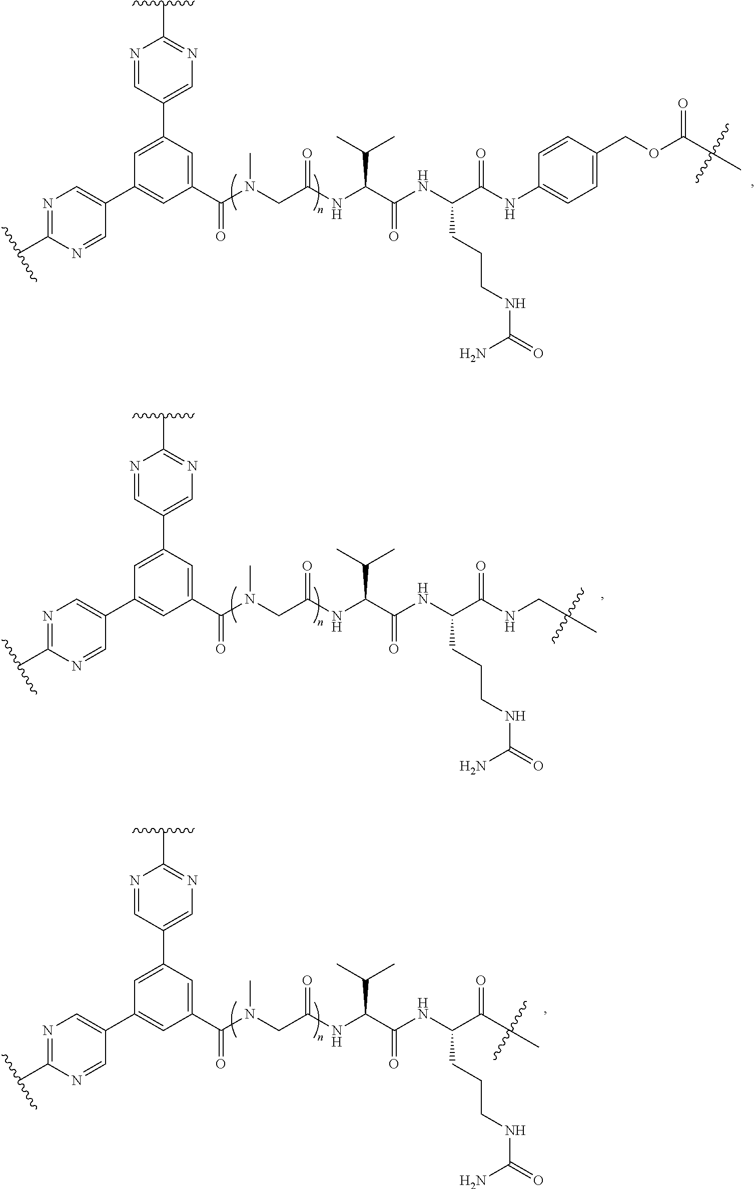

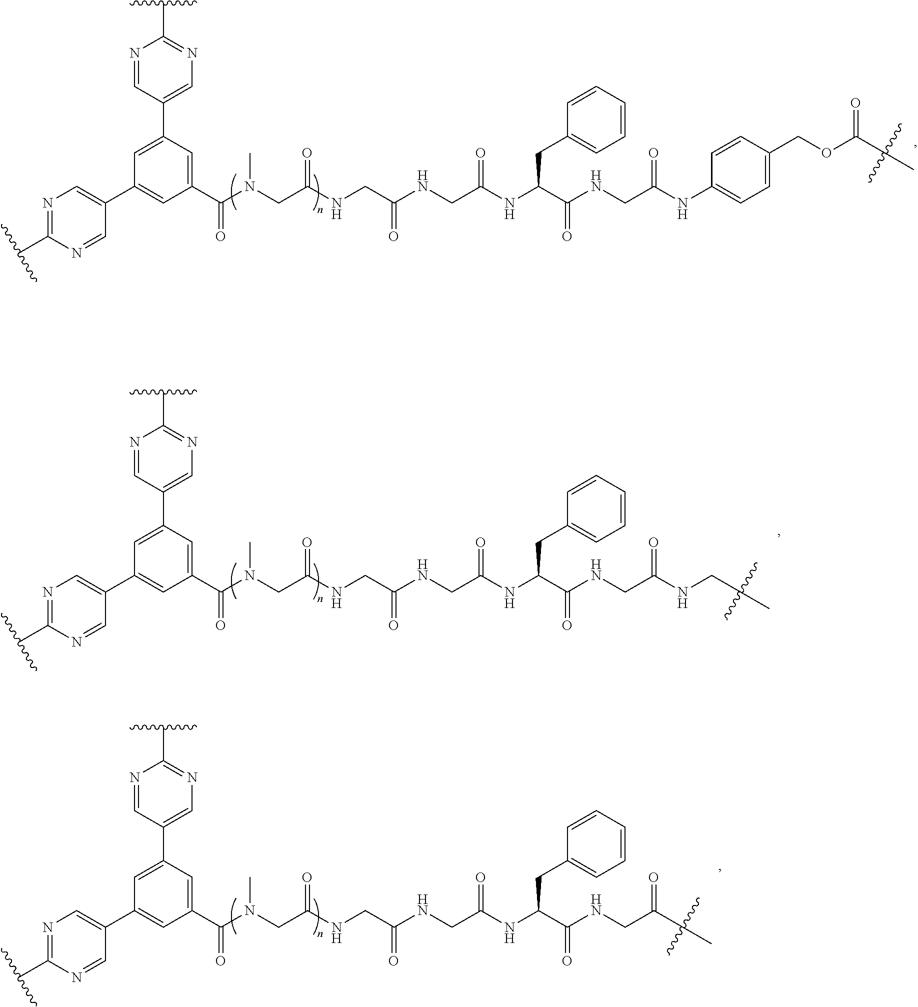

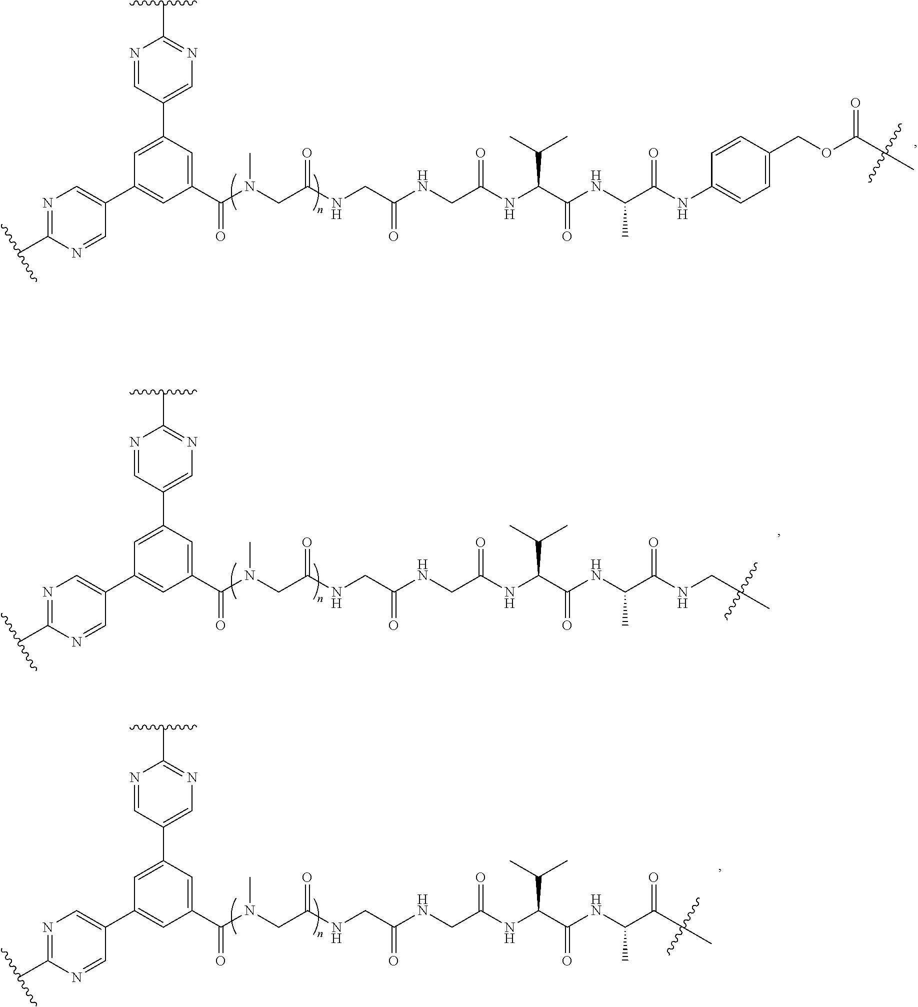

n is an integer selected from 1 to 20. In some embodiments, n is an integer selected from 1 to 15, such as an integer of 1 to 12, 3 to 12, for example, n is 1, 2, 3, 4, 5, 6, 7, 8, 9, 10, 11, 12, 13, 14 or 15.

In some embodiments,

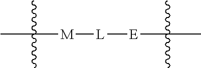

is selected from the group consisting of the following substituted or unsubstituted structures:

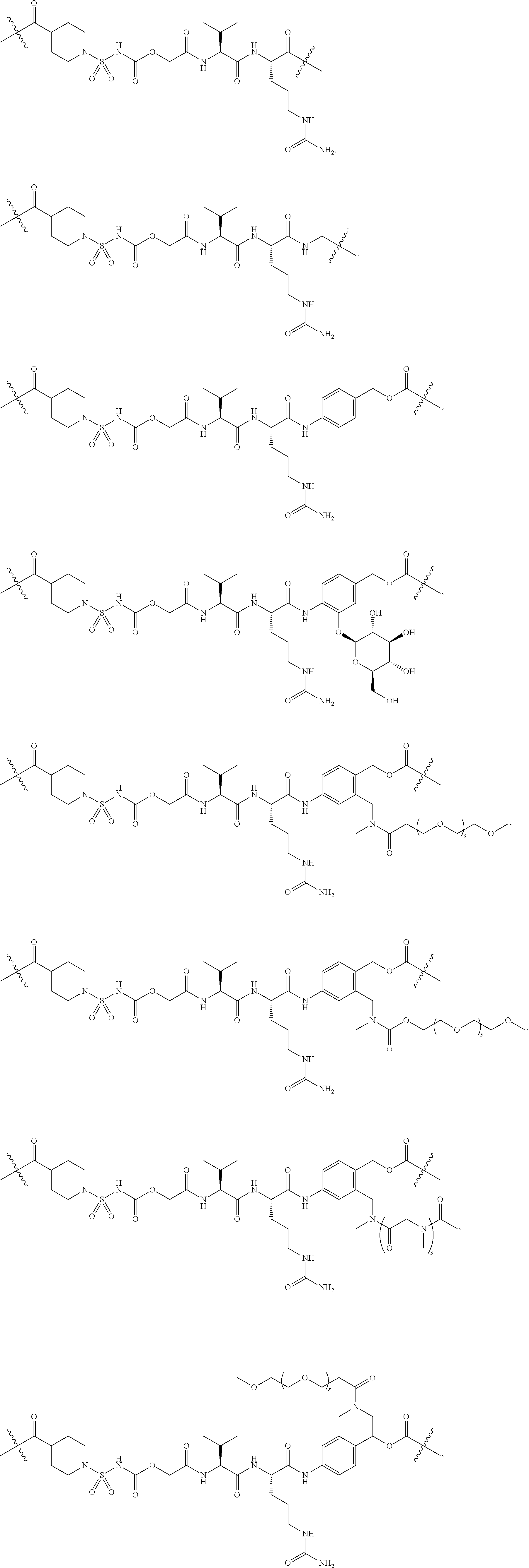

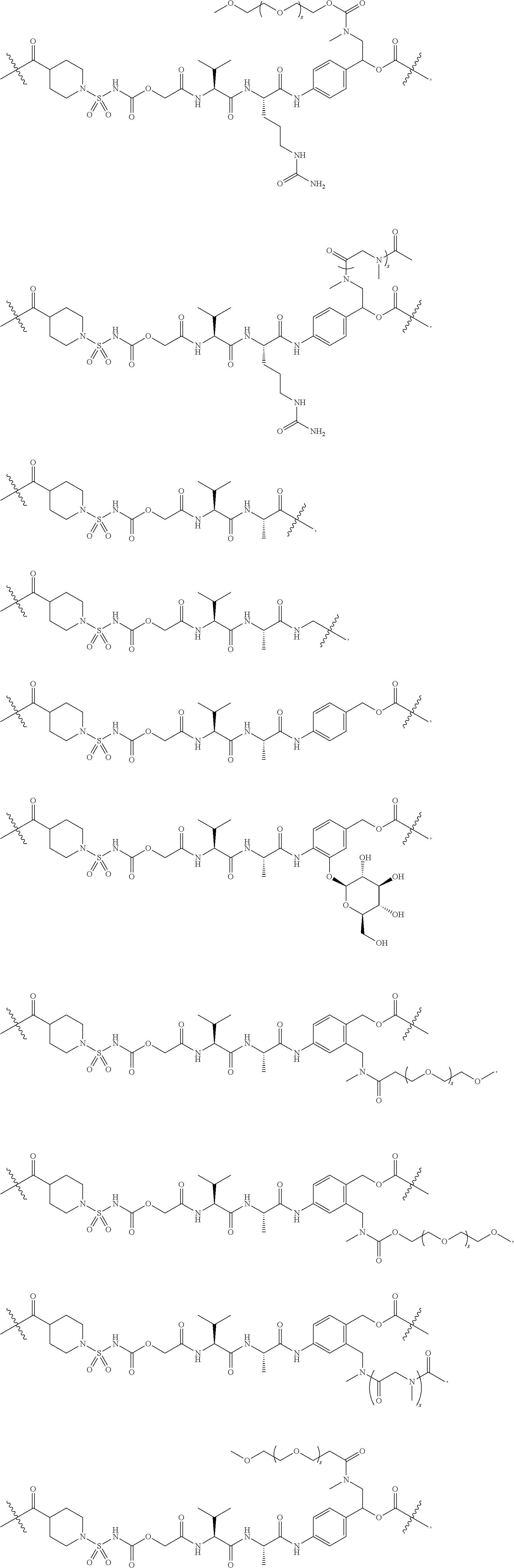

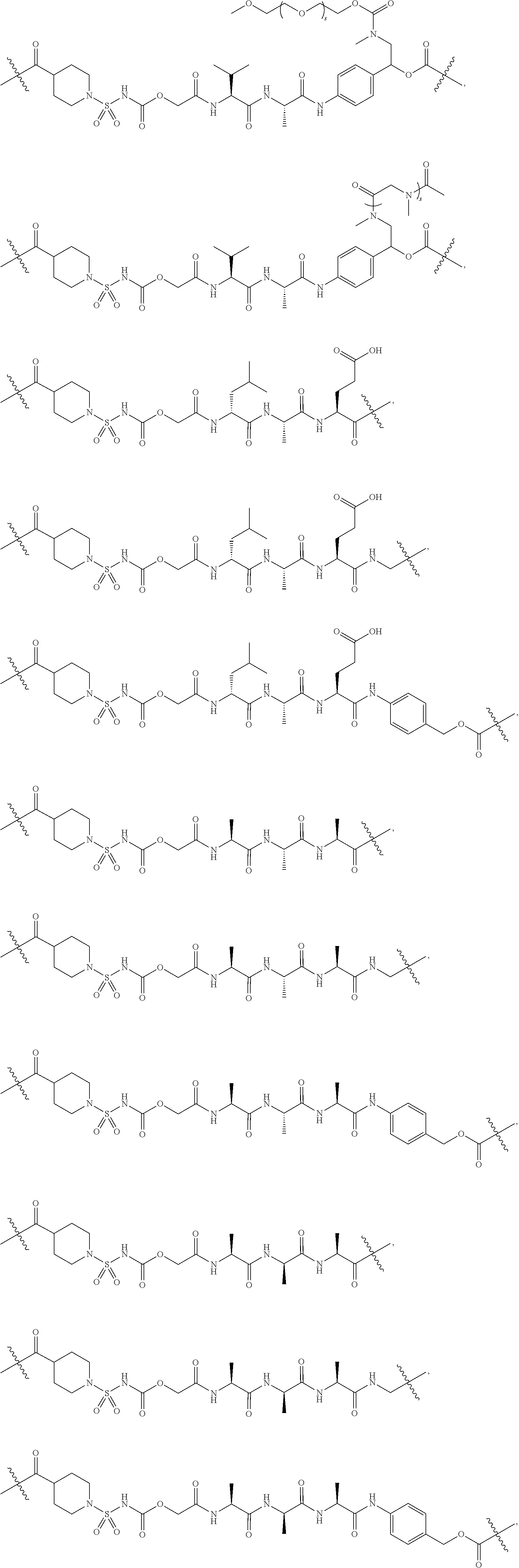

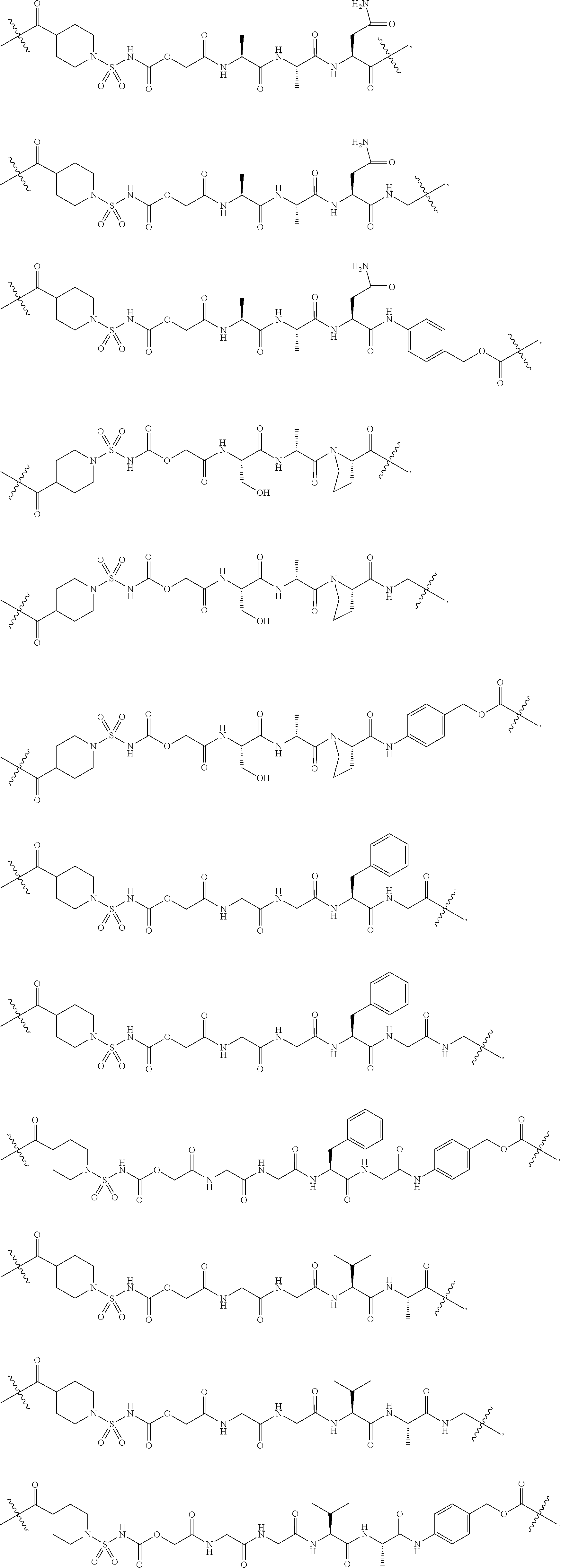

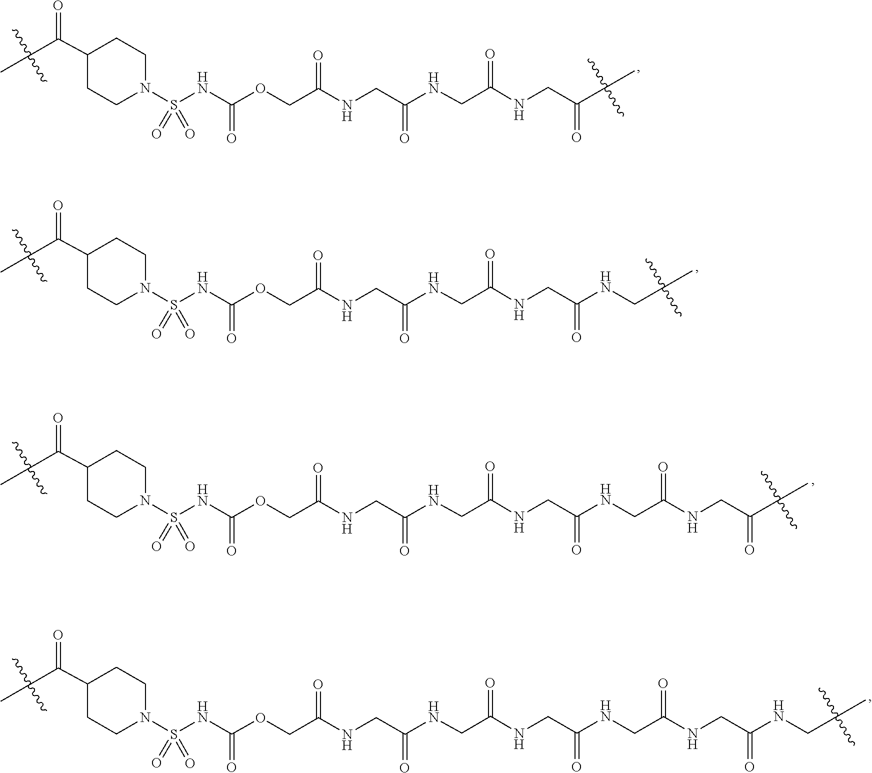

s is an integer selected from 1 to 20. In some embodiments, s is an integer selected from 1 to 15, such as an integer of 1 to 12, 3 to 12, for example, s is 1, 2, 3, 4, 5, 6, 7, 8, 9, 10, 11, 12, 13, 14 or 15.

In some embodiments,

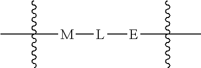

is selected from the group consisting of the following substituted or unsubstituted structures:

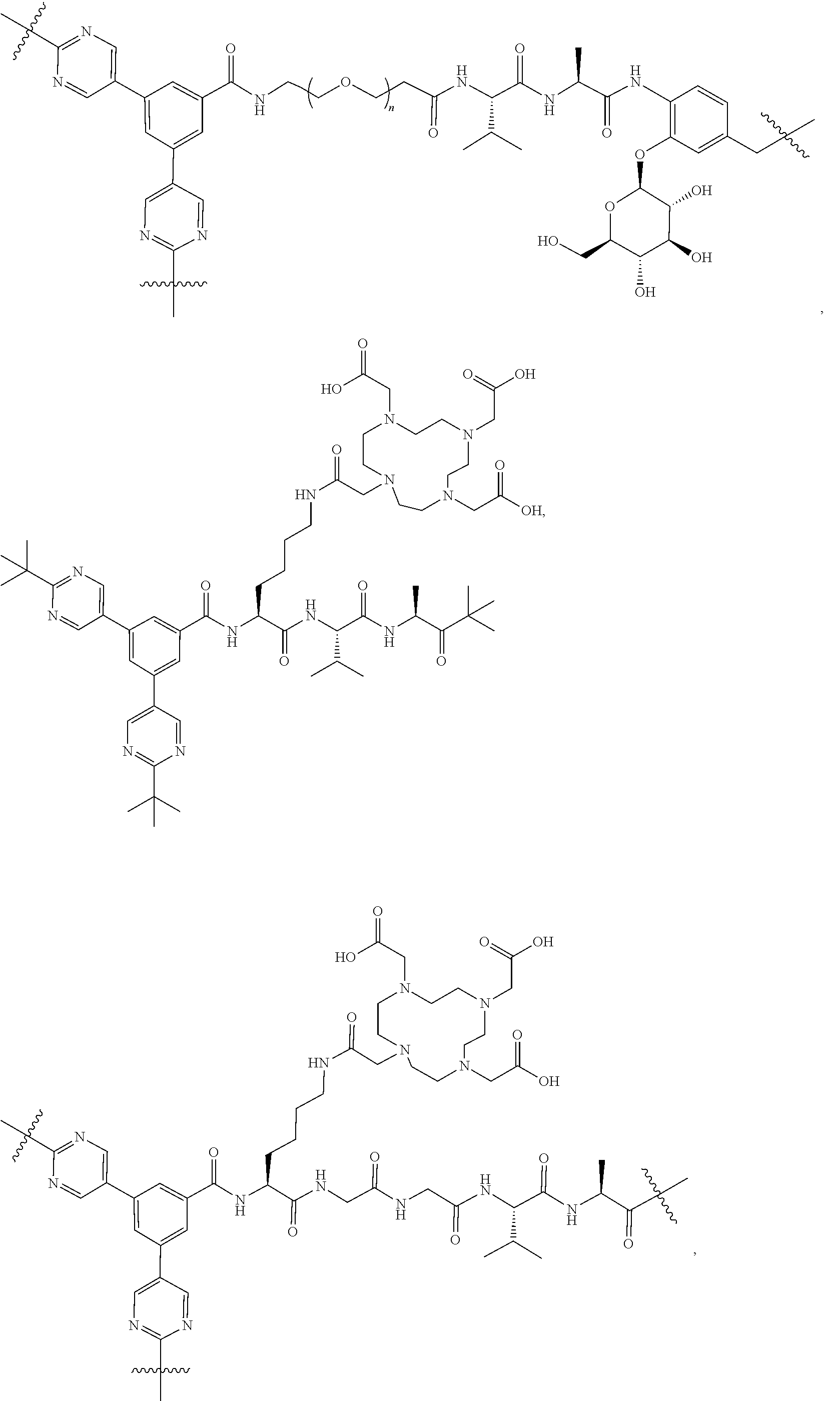

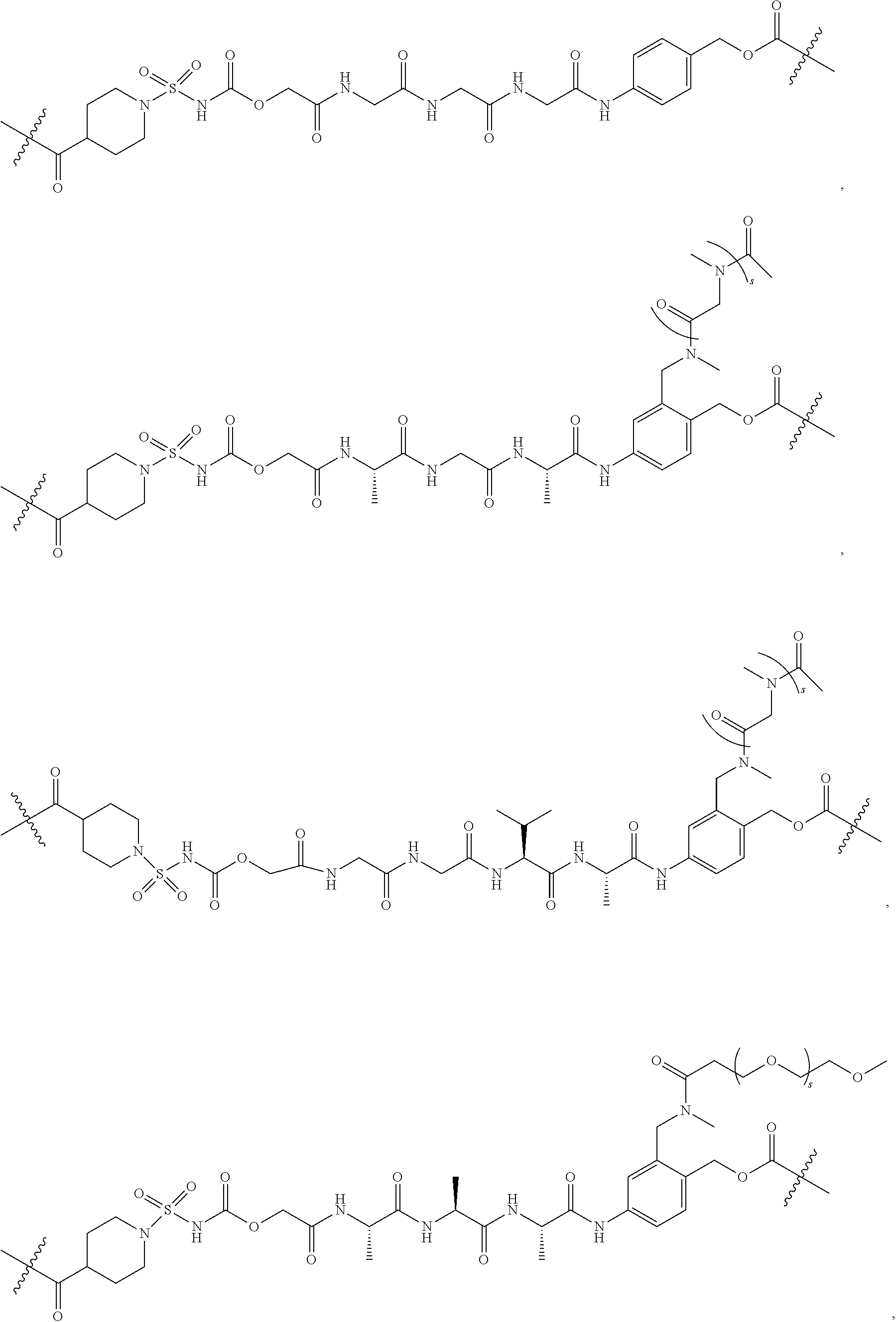

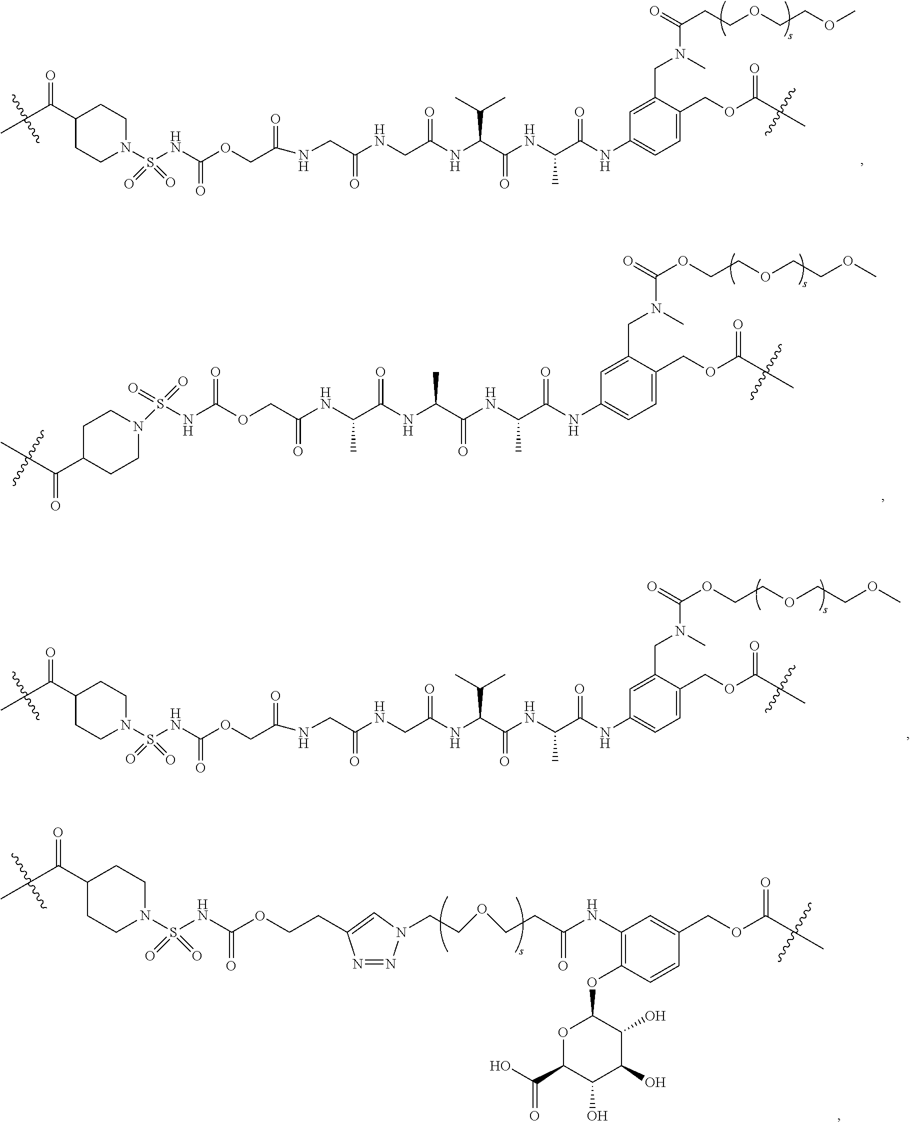

n and s are each independently an integer selected from 1 to 20. In some embodiments, n and s are each independently an integer selected from 1 to 15, such as an integer of 1 to 12, 3 to 12, for example, 1, 2, 3, 4, 5, 6, 7, 8, 9, 10, 11, 12, 13, 14 or 15.

In some embodiments,

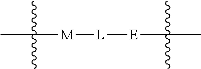

is selected from the group consisting of the following substituted or unsubstituted structures:

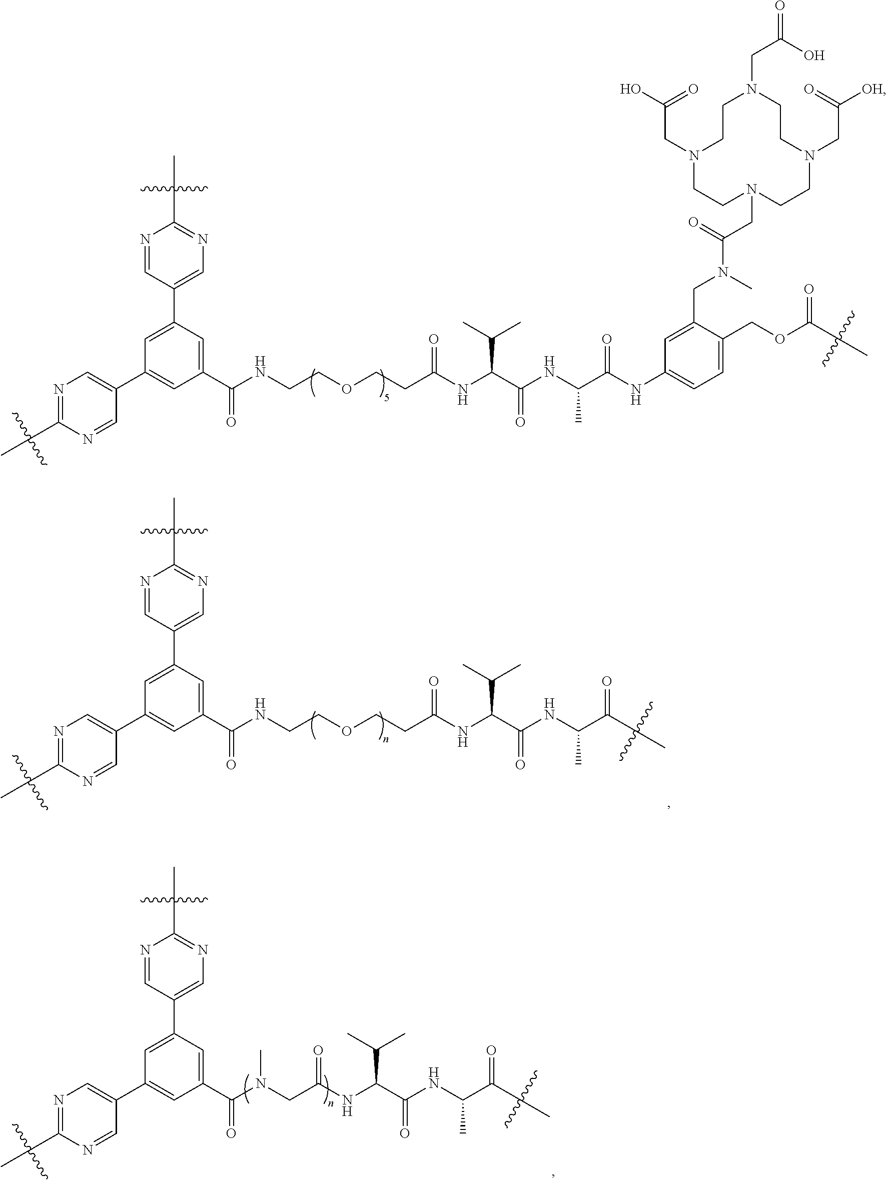

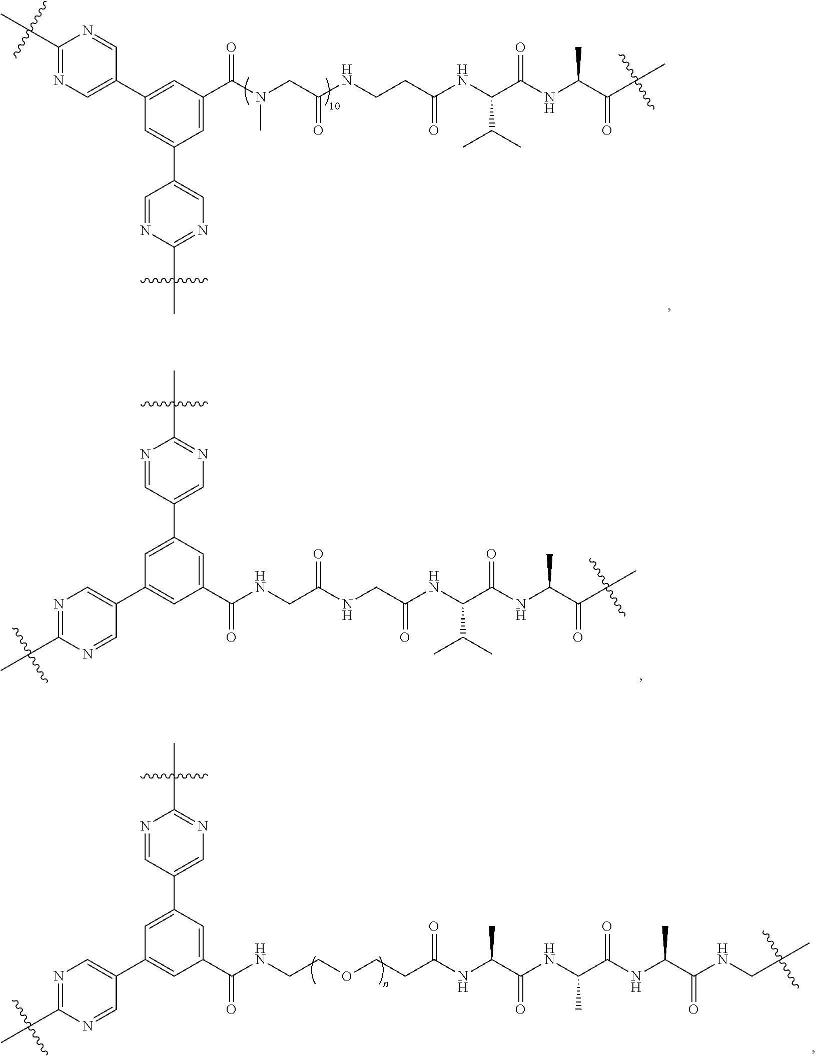

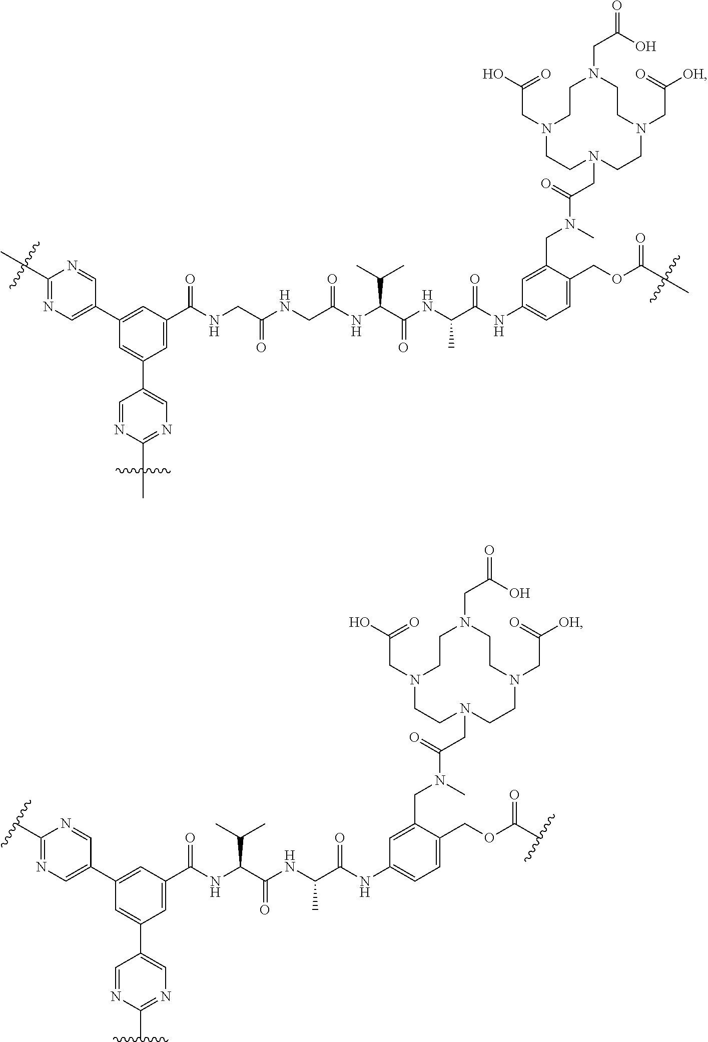 [SMILES: *C(=O)OCc1ccc(NC(=O)[C@H](C)NC(=O)[C@@H](NC(=O)CNC(=O)CNC(=O)c2cc(-c3cnc(*)nc3)cc(-c3cnc(*)nc3)c2)C(C)C)cc1CN(C)C(=O)CN1CCN(CC(=O)O)CCN(CC(=O)O)CCN(CC(=O)O)CC1 ; *C(=O)OCc1ccc(NC(=O)[C@H](C)NC(=O)[C@@H](NC(=O)c2cc(-c3cnc(*)nc3)cc(-c3cnc(*)nc3)c2)C(C)C)cc1CN(C)C(=O)CN1CCN(CC(=O)O)CCN(CC(=O)O)CCN(CC(=O)O)CC1]

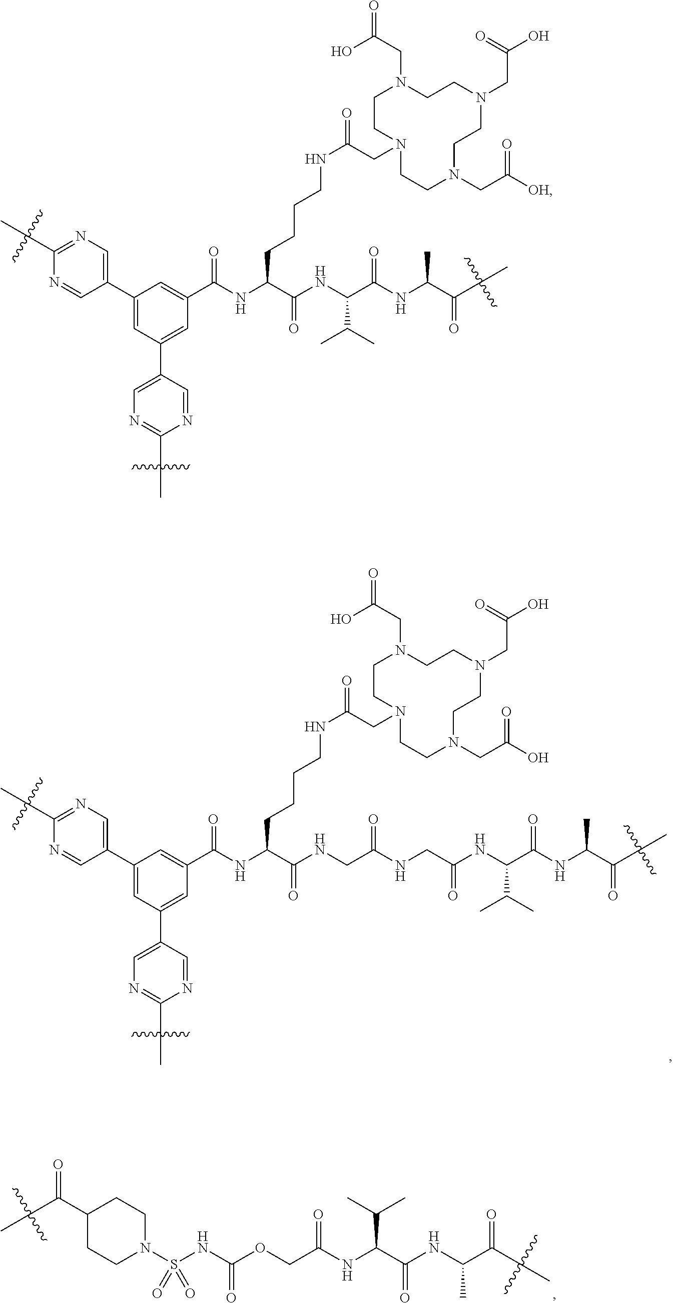 [SMILES: *C(=O)[C@H](C)NC(=O)[C@@H](NC(=O)[C@H](CCCCNC(=O)CN1CCN(CC(=O)O)CCN(CC(=O)O)CCN(CC(=O)O)CC1)NC(=O)c1cc(-c2cnc(*)nc2)cc(-c2cnc(*)nc2)c1)C(C)C ; *C(=O)[C@H](C)NC(=O)[C@@H](NC(=O)CNC(=O)CNC(=O)[C@H](CCCCNC(=O)CN1CCN(CC(=O)O)CCN(CC(=O)O)CCN(CC(=O)O)CC1)NC(=O)c1cc(-c2cnc(*)nc2)cc(-c2cnc(*)nc2)c1)C(C)C ; *C(=O)C1CCN(S(=O)(=O)NC(=O)OCC(=O)N[C@H](C(=O)N[C@@H](C)C(*)=O)C(C)C)CC1]

n is an integer selected from 1 to 20, preferably an integer selected from 1 to 15, such as an integer of 1 to 12, 3 to 12, such as 1, 2, 3, 4, 5, 6, 7, 8, 9, 10, 11, 12, 13, 14 or 15, preferably 5, 8, 10.

In some embodiments,

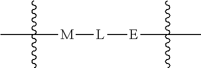

is selected from the group consisting of the following substituted or unsubstituted structures:

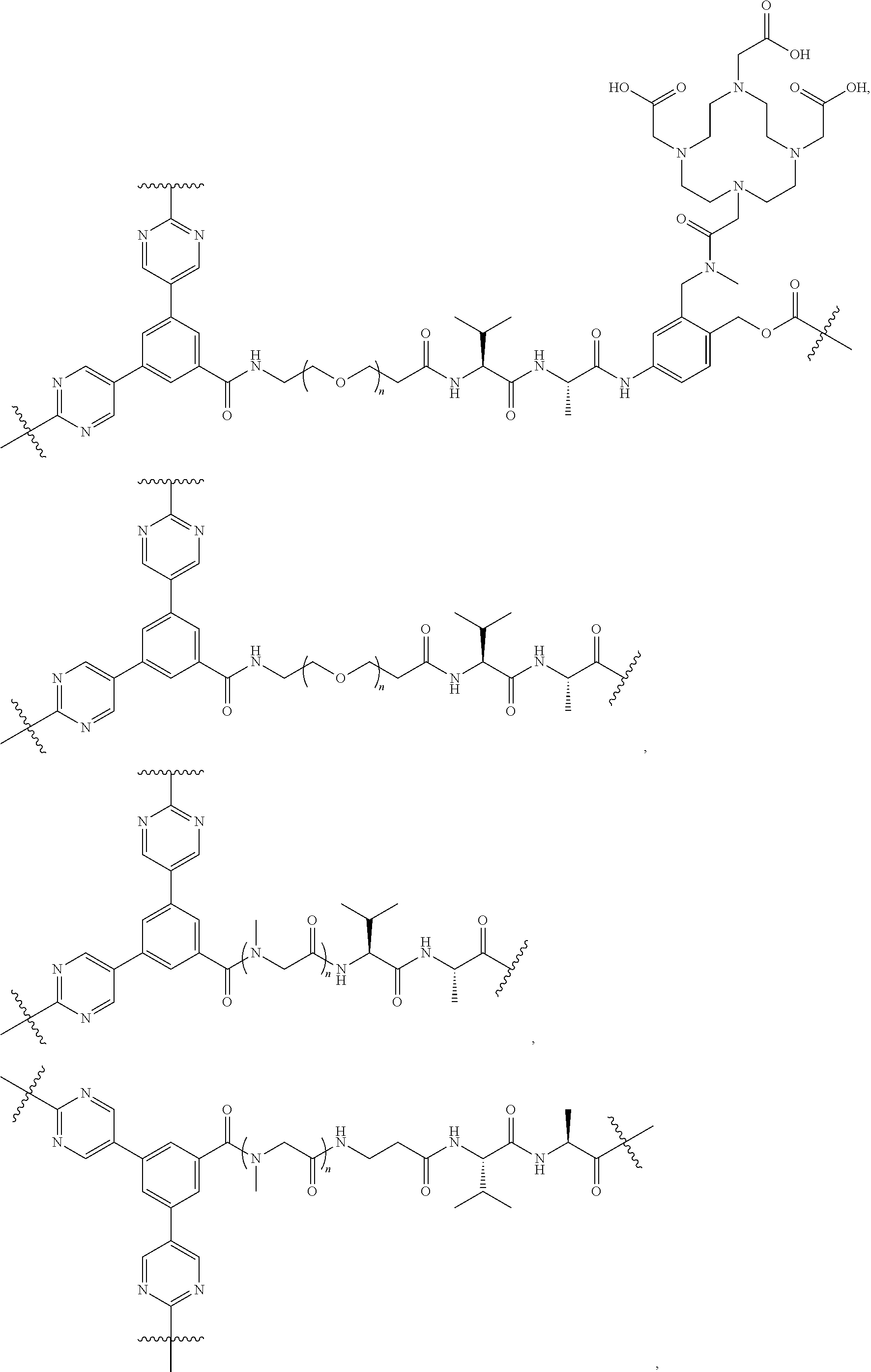

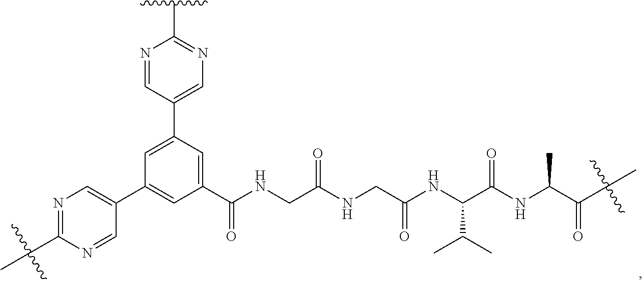 [SMILES: *C(=O)[C@H](C)NC(=O)[C@@H](NC(=O)CNC(=O)CNC(=O)c1cc(-c2cnc(*)nc2)cc(-c2cnc(*)nc2)c1)C(C)C]

n is an integer selected from 1 to 20, such as an integer of 1 to 12, 3 to 12, for example, n is 1, 2, 3, 4, 5, 6, 7, 8, 9, 10, 11, 12, 13, 14 or 15, preferably 5, 8, 10.

In some embodiments,

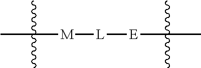

is selected from the group consisting of the following substituted or unsubstituted structures:

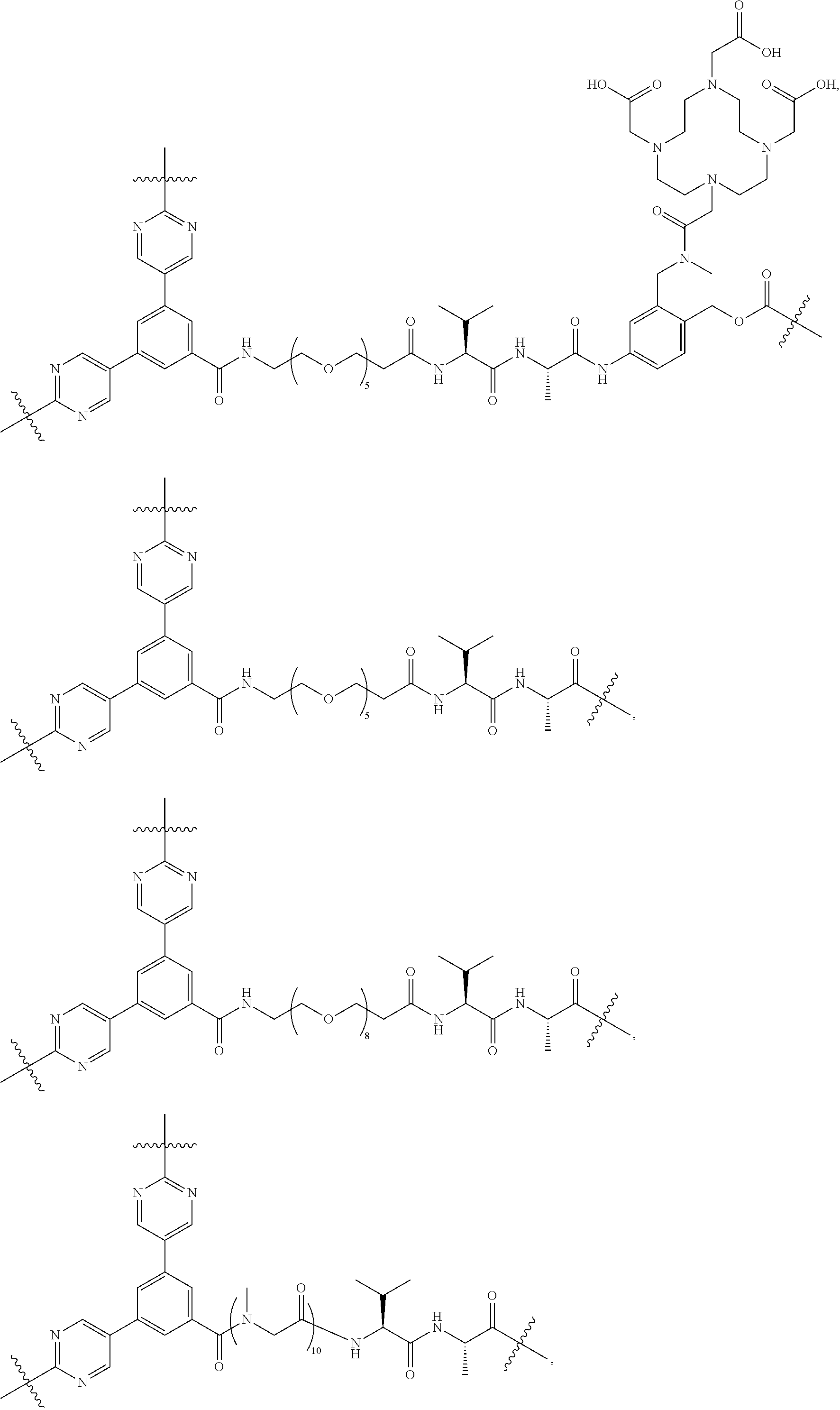

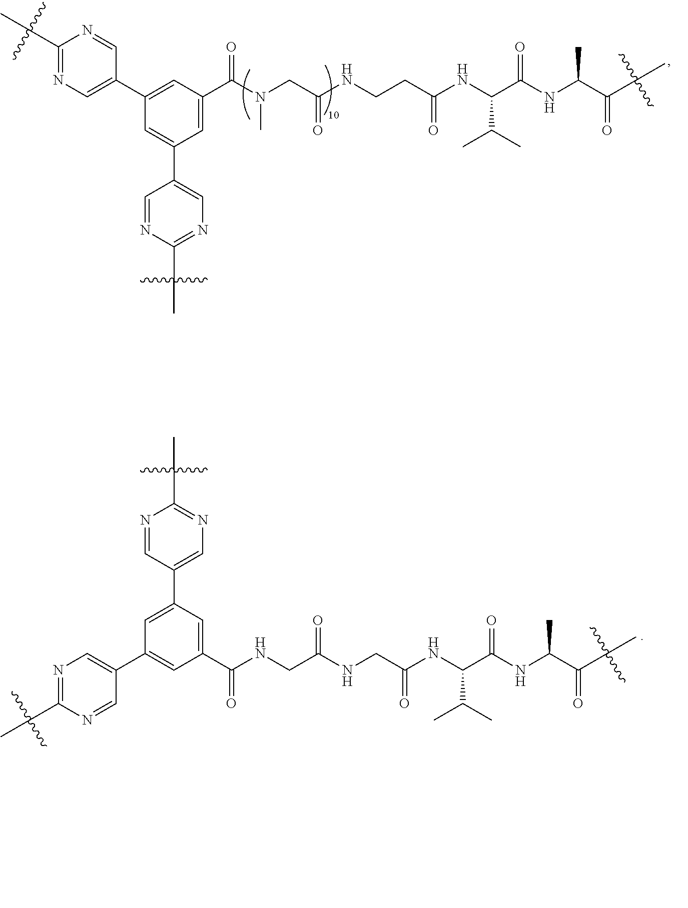

In some embodiments,

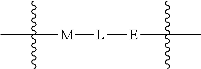

is selected from the group consisting of the following substituted or unsubstituted structures:

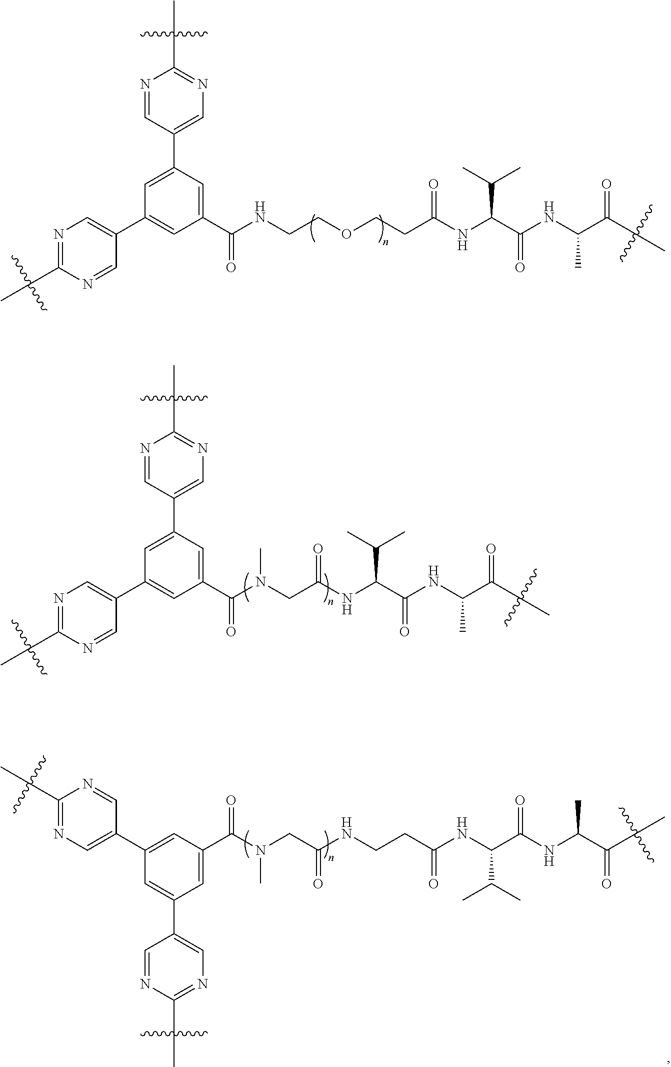

wherein n is an integer selected from 1 to 20, such as an integer of 1 to 12, 3 to 12, for example, n is 1, 2, 3, 4, 5, 6, 7, 8, 9, 10, 11, 12, 13, 14 or 15, preferably 5, 8, 10.

In some embodiments,

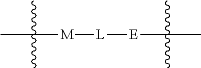

is selected from the group consisting of the following substituted or unsubstituted structures:

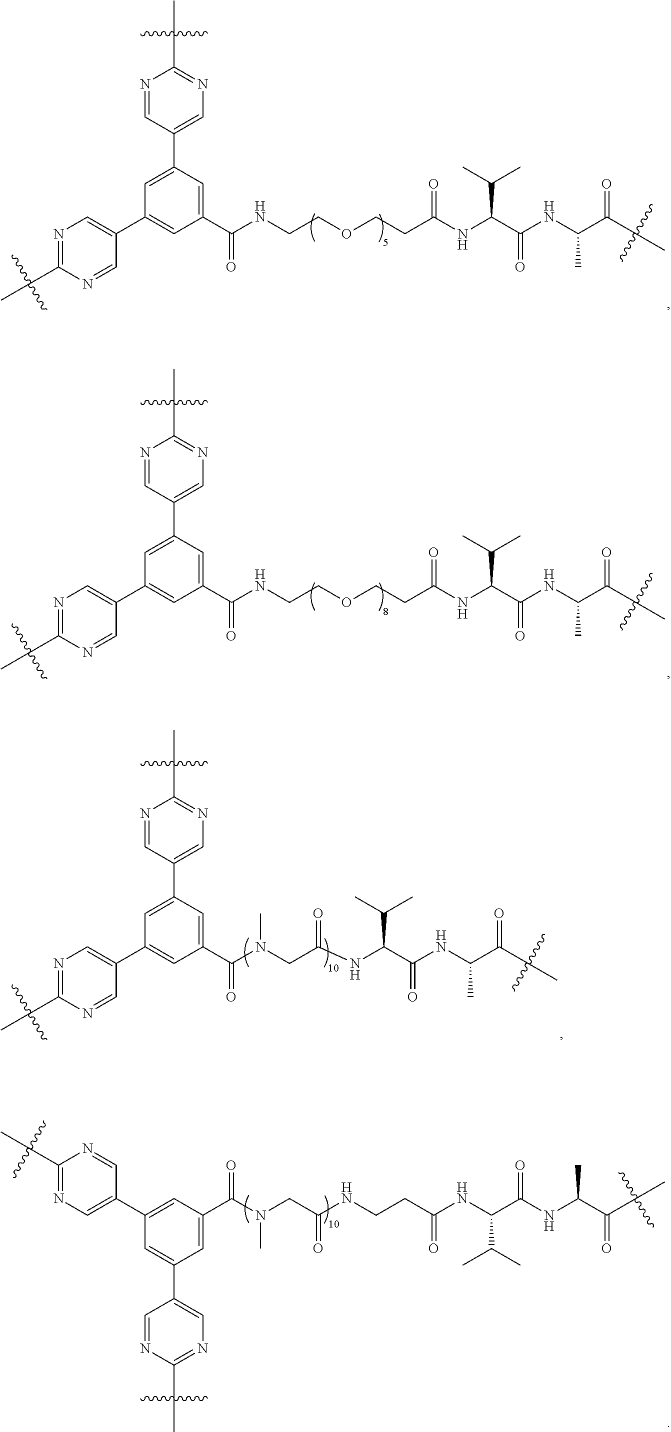

In some embodiments, the cytotoxic drug is selected from the group consisting of anti-tubulin agents, DNA intercalators, DNA topoisomerase inhibitors, RNA polymerase inhibitors and gene transcription inhibitors. In some embodiments, the tubulin inhibitor is an auristatin compound, a maytansine compound, or an eribulin compound. In some embodiments, the DNA intercalator is a pyrrolobenzodiazepine (PB3D) compound, trabectedin, or lurbinectedin. In some embodiments, the DNA topoisomerase inhibitor is a topoisomerase I inhibitor (e.g., camptothecin, hydroxycamptothecin, 9-aminocamptothecin, SN-38, irinotecan, exatecan, topotecan, belotecan, rubitecan, diflomotecan, lurtotecan, karenitecin, gimatecan, namitecan, simmitecan, chimmitecan, silatecan, or elomotecan) or a topoisomerase II inhibitor (e.g., adriamycin, doxorubicin, PNU-159682 and analogs thereof, duocarmycin, daunorubicin, mitoxantrone, podophyllotoxin, or etoposide). In some embodiments, the RNA polymerase inhibitor is a-amanitin. In some embodiments, the gene transcription inhibitor is triptolide and pharmaceutically acceptable salts, esters, and analogs thereof.

In some embodiments, the cytotoxic drug is a topoisomerase I inhibitor (e.g., camptothecin, hydroxycamptothecin, 9-aminocamptothecin, SN-38, irinotecan, exatecan, topotecan, belotecan, rubitecan, diflomotecan, lurtotecan, karenitecin, gimatecan, namitecan, simmitecan, chimmitecan, silatecan, or elomotecan).

The cytotoxic drug disclosed in the present application typically comprises a variety of functional groups, such as hydroxyl (—OH), carboxyl (—COOH), primary amino (—NH₂), secondary amino (—NR₁H), tertiary amino (—NR₂R₃), wherein R₁, R₂, R₃ here represent only non-hydrogen substituents on N, or sulfhydryl (—SH), and the functional group can react with a suitable functional group in the rest of the conjugate to achieve connection.

In some embodiments, the cytotoxic drug is connected through —OH, primary amino, secondary amino or tertiary amino, or —SH thereon to E in the antibody-drug conjugate. In some embodiments, D is a monovalent structure obtained by losing one H from —OH, —NH₂ or secondary amino on the cytotoxic drug.

In some embodiments, the cytotoxic drug is selected from the group consisting of the following compounds or pharmaceutically acceptable salts, isotopically labeled compounds, solvates, hydrates, or isomers thereof, or any crystalline forms or racemates thereof:

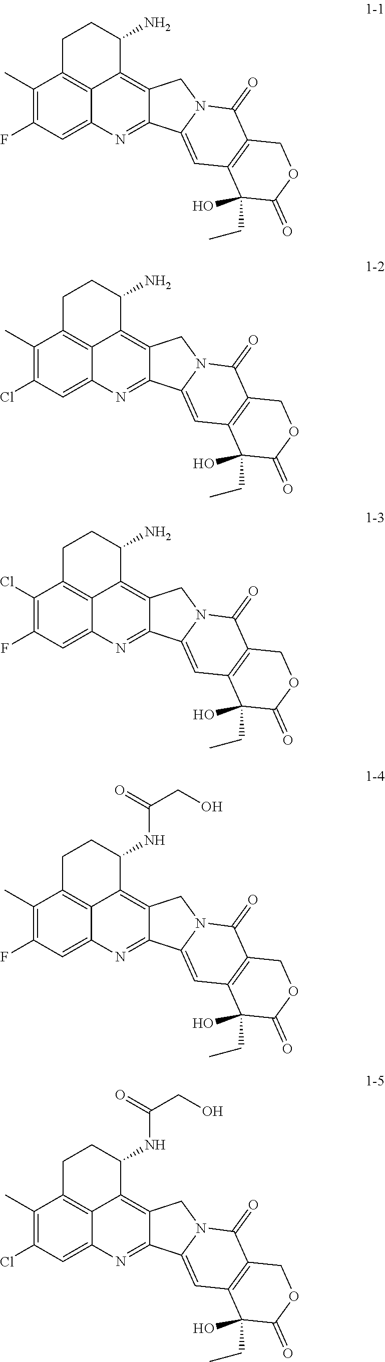 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

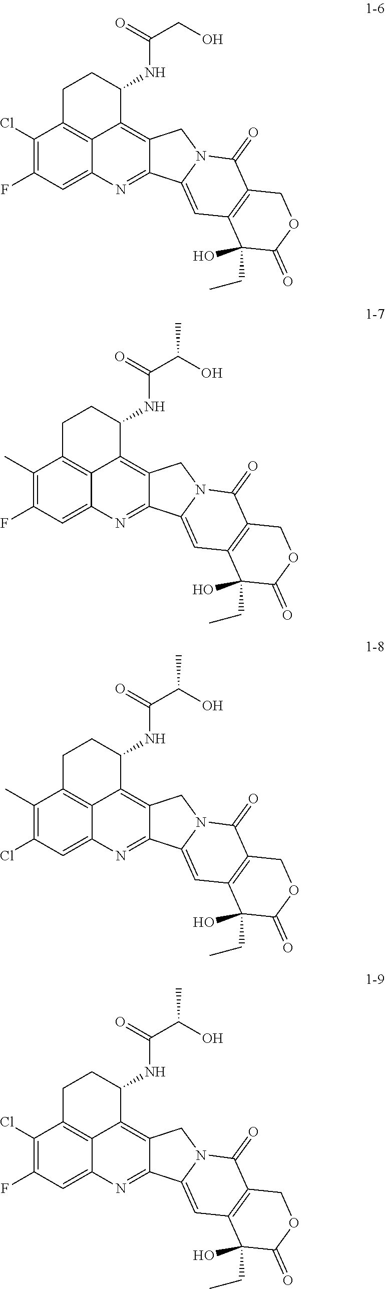

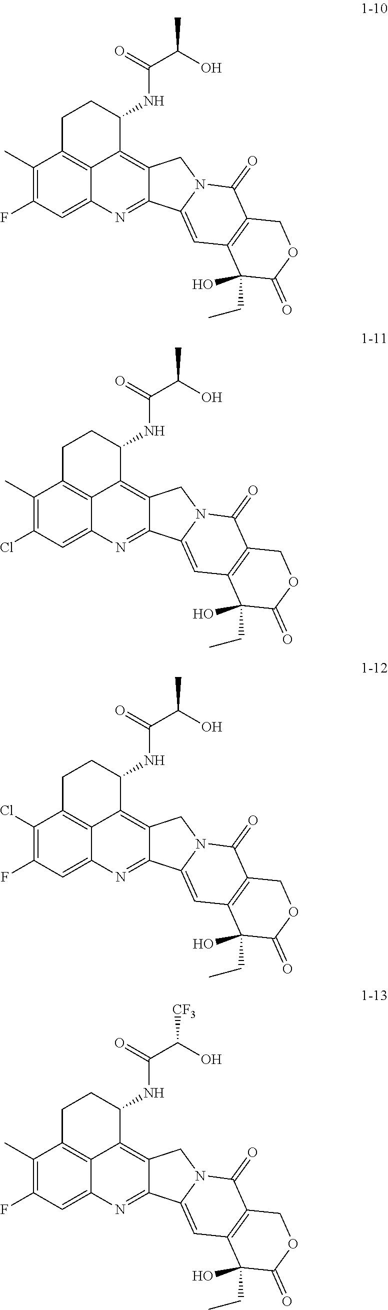

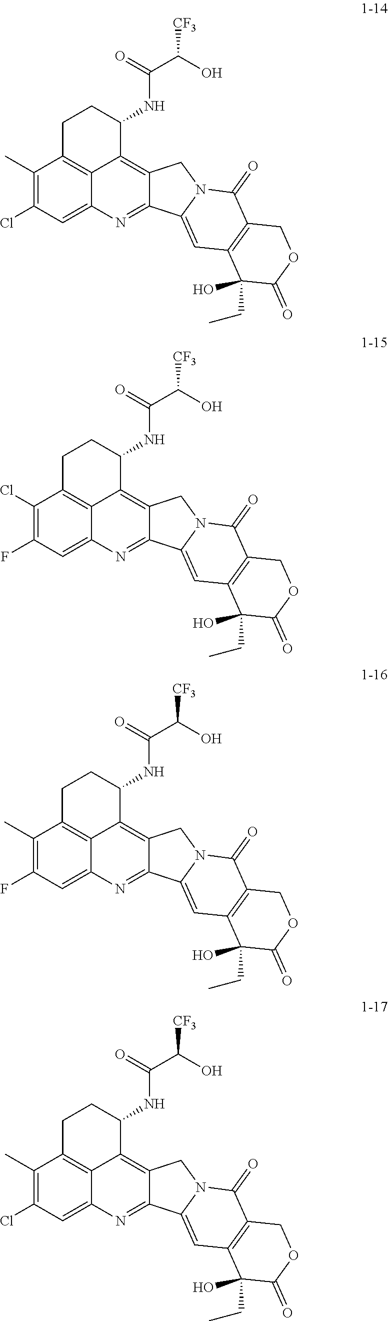

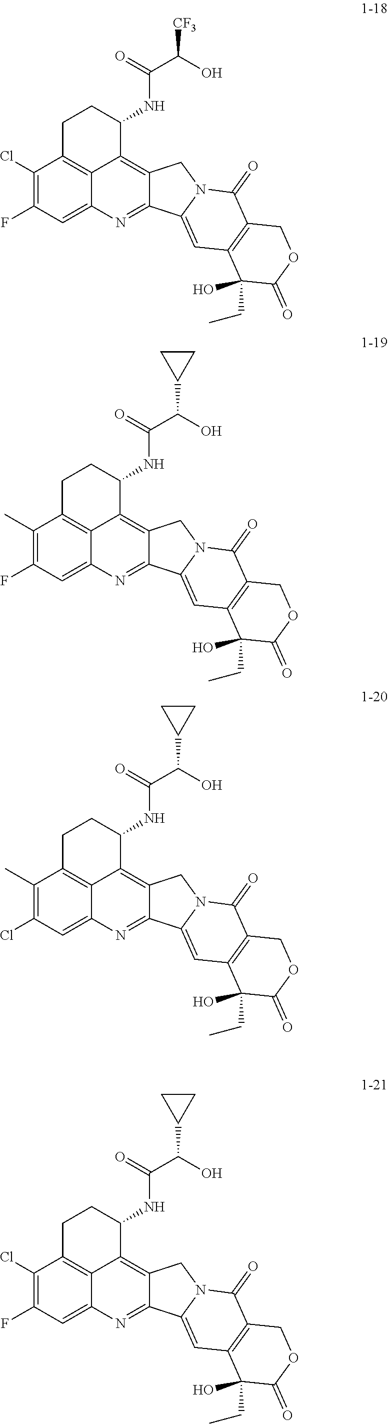

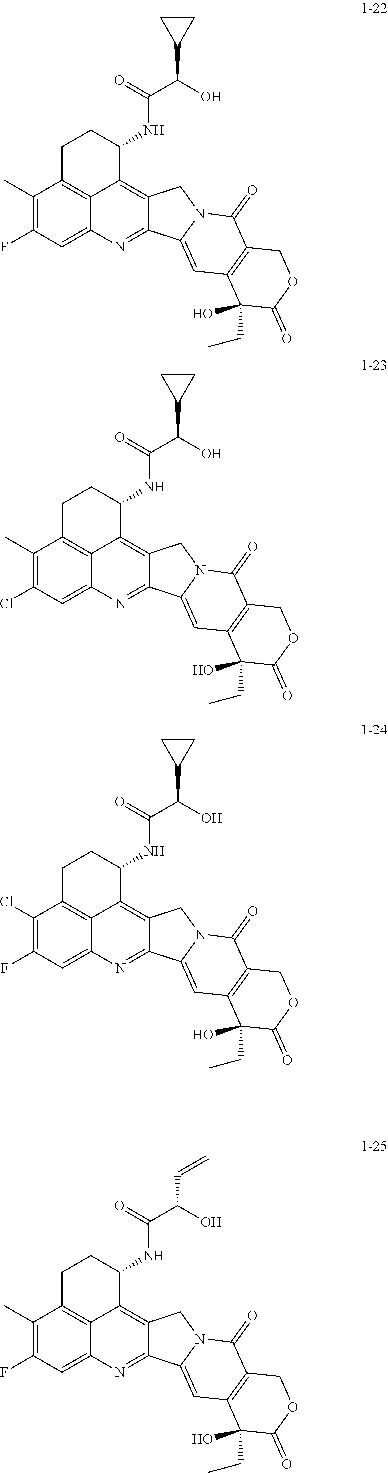

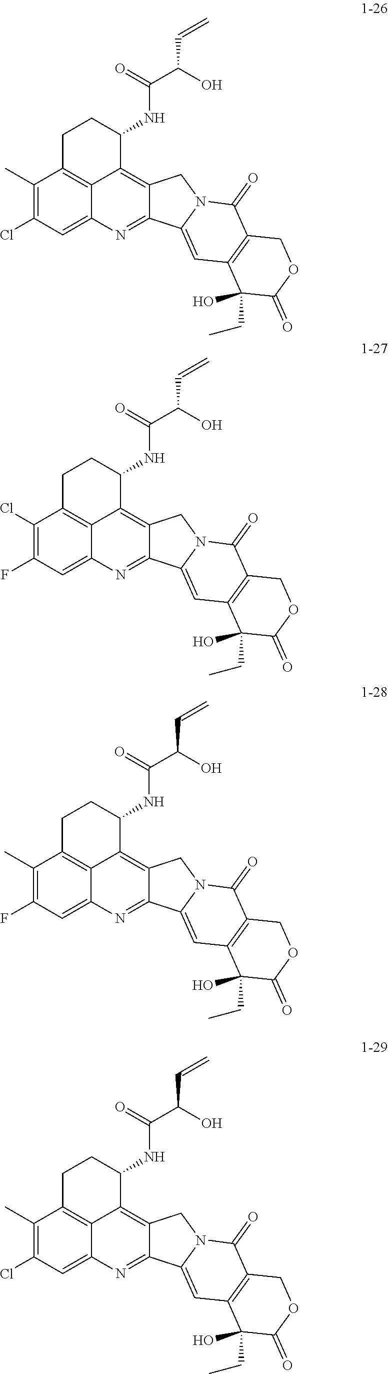

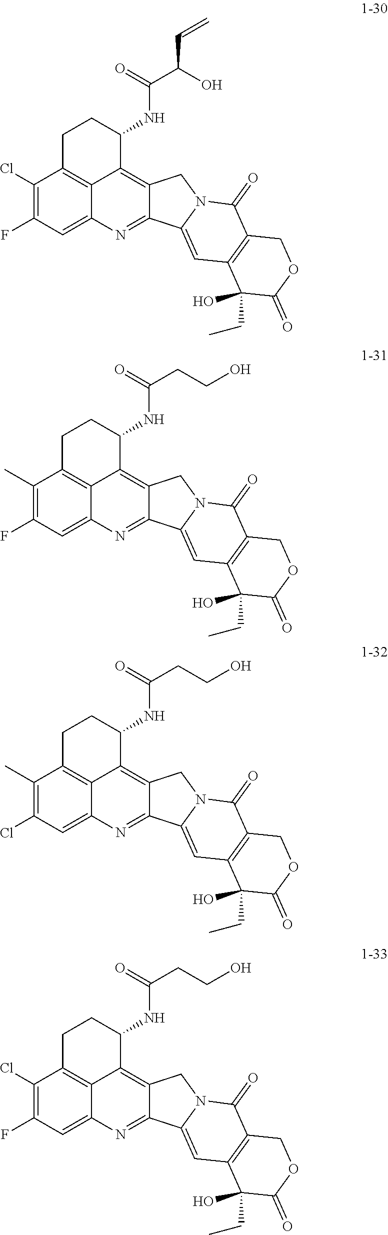

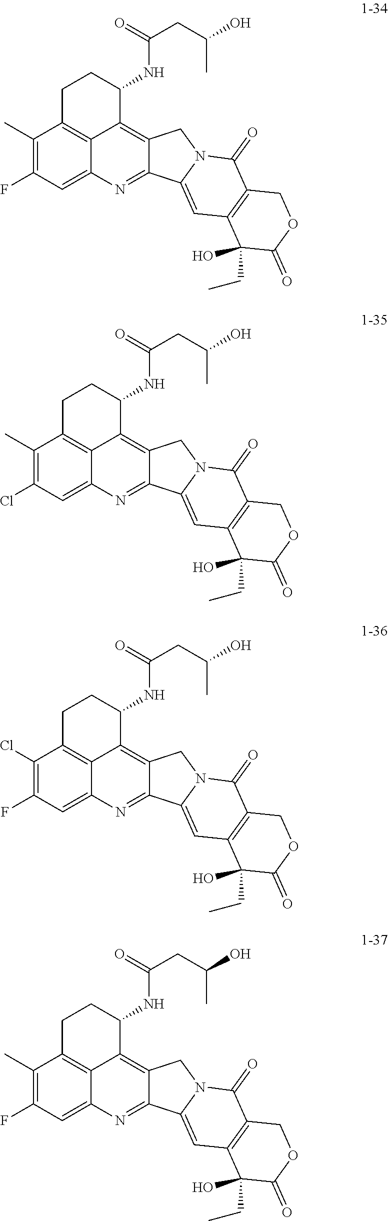 [SMILES: CC[C@@]1(O)C(=O)OCc2c1cc1n(c2=O)Cc2c-1nc1cc(F)c(C)c3c1c2[C@@H](NC(=O)C[C@@H](C)O)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(C)c3c1c2[C@@H](NC(=O)C[C@H](C)O)CC3]

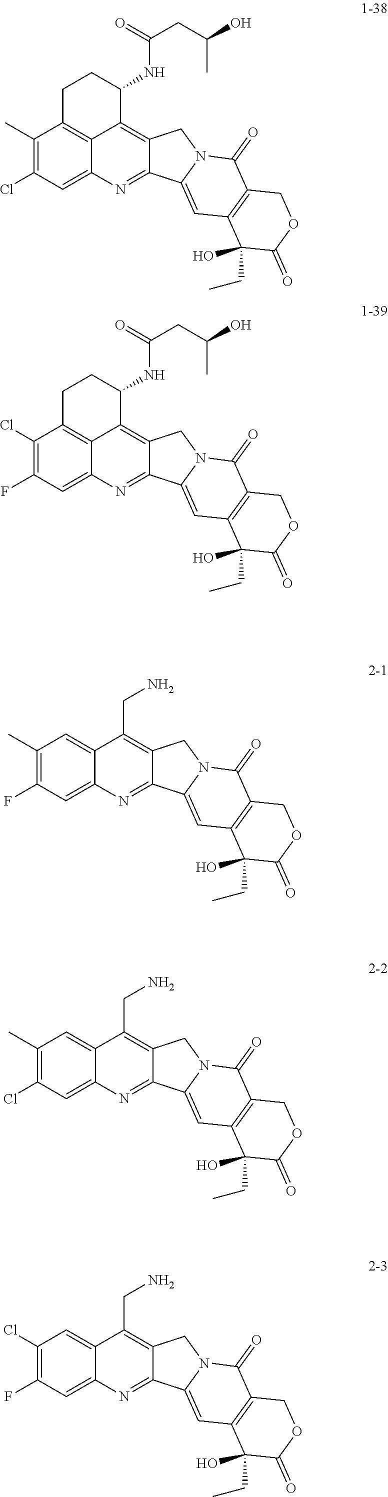

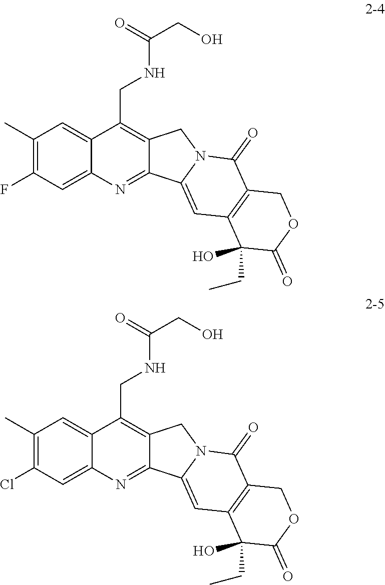

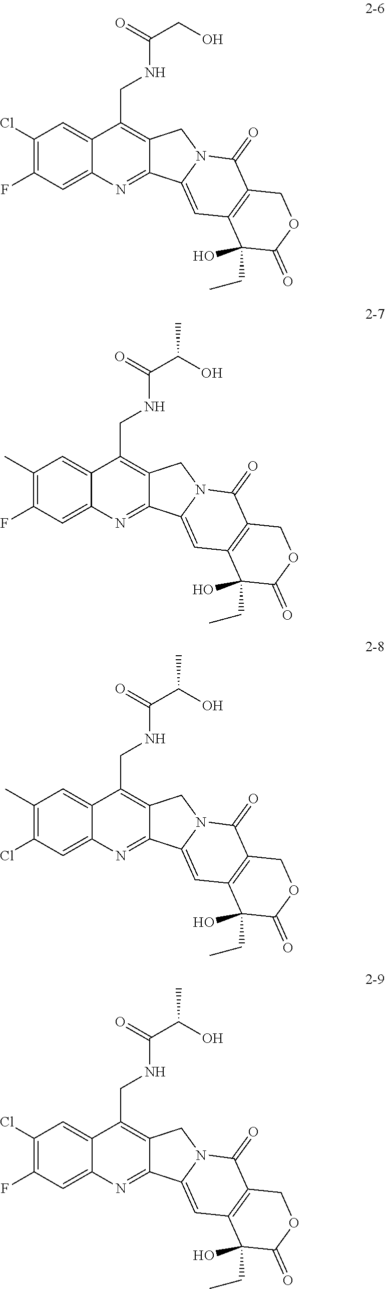

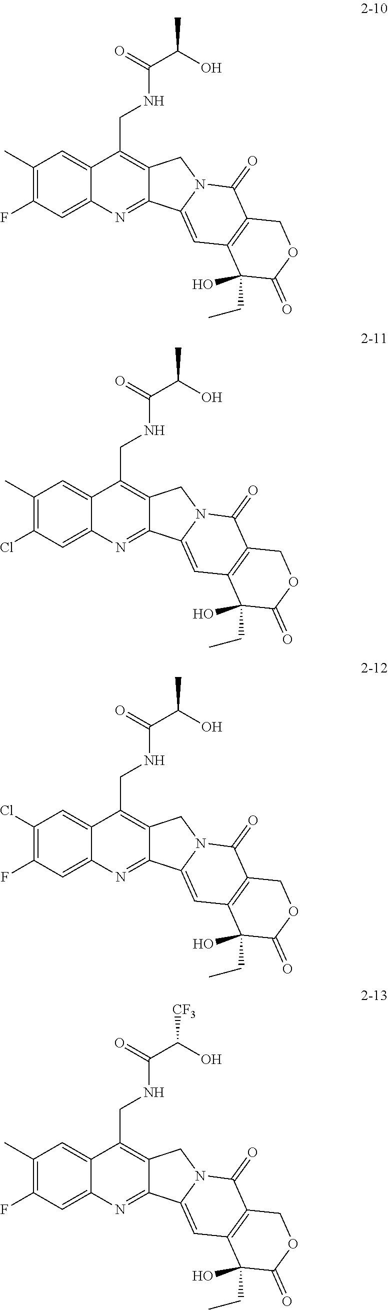

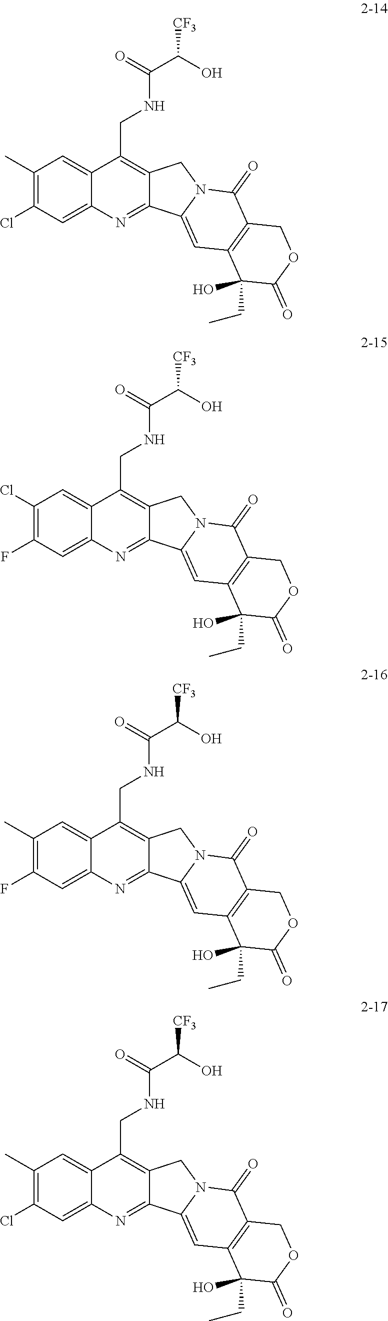

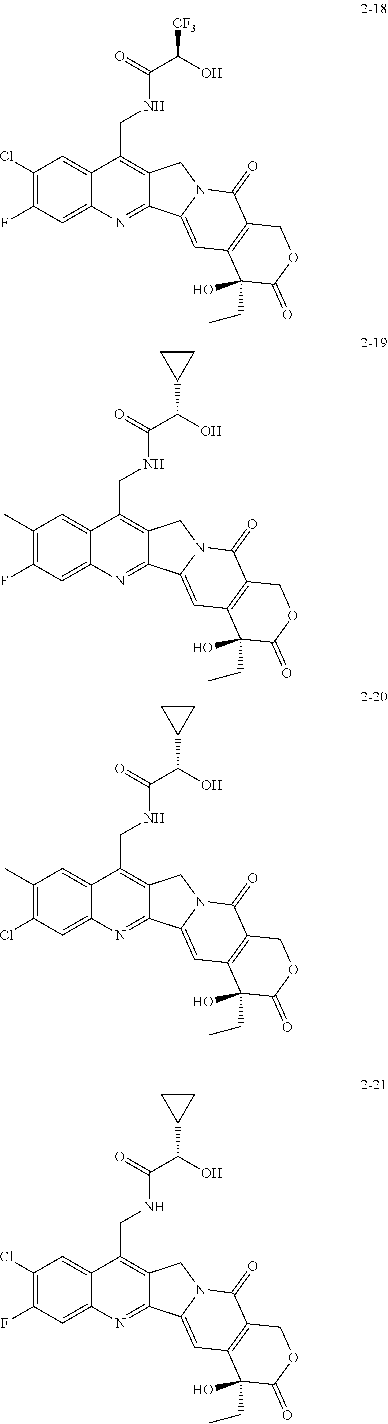

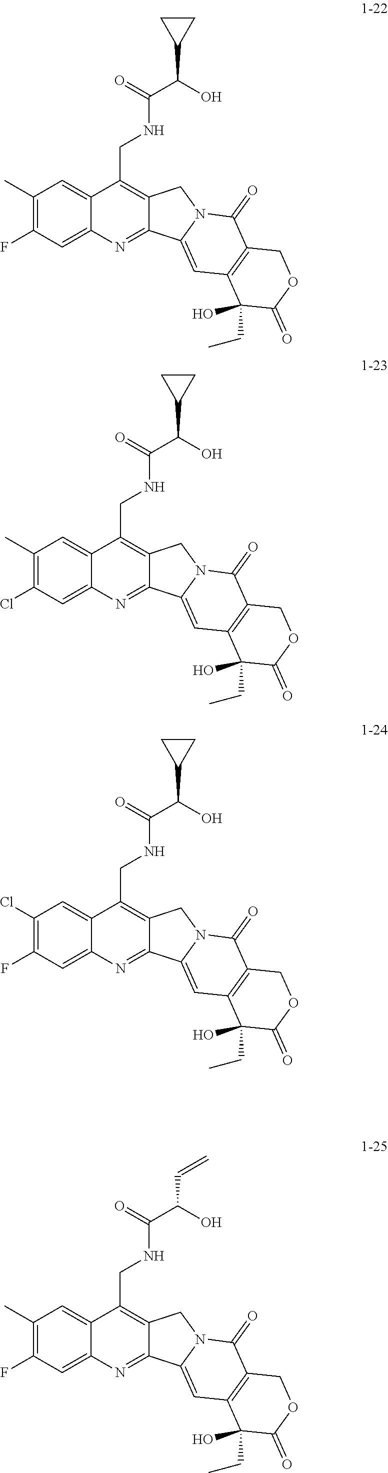

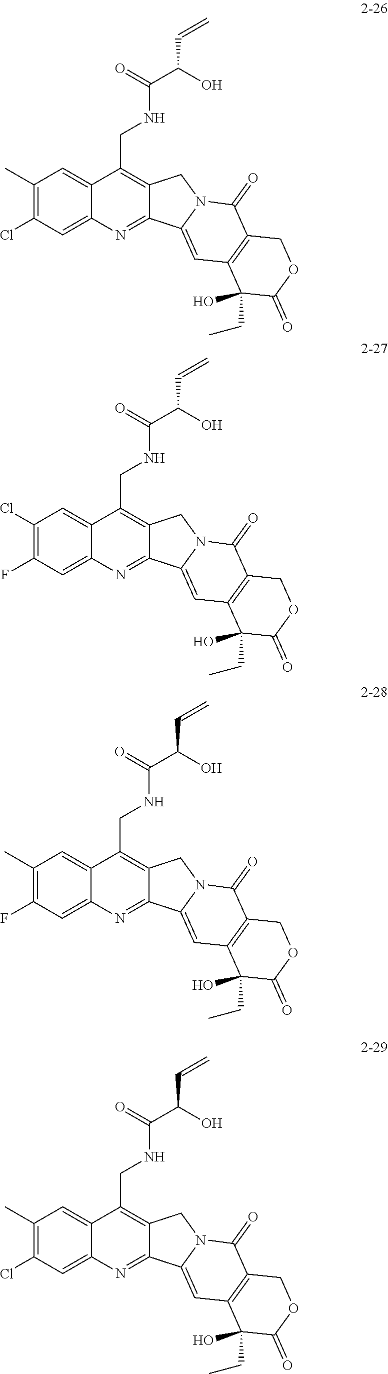

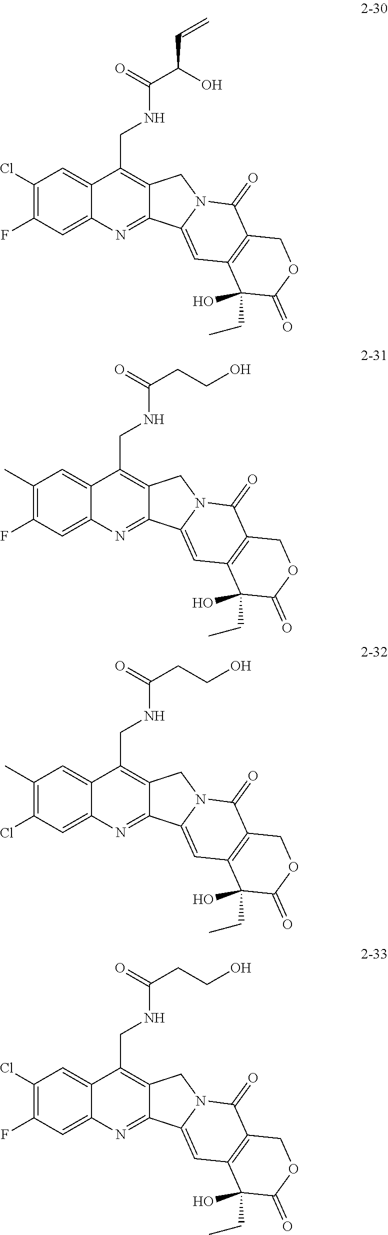

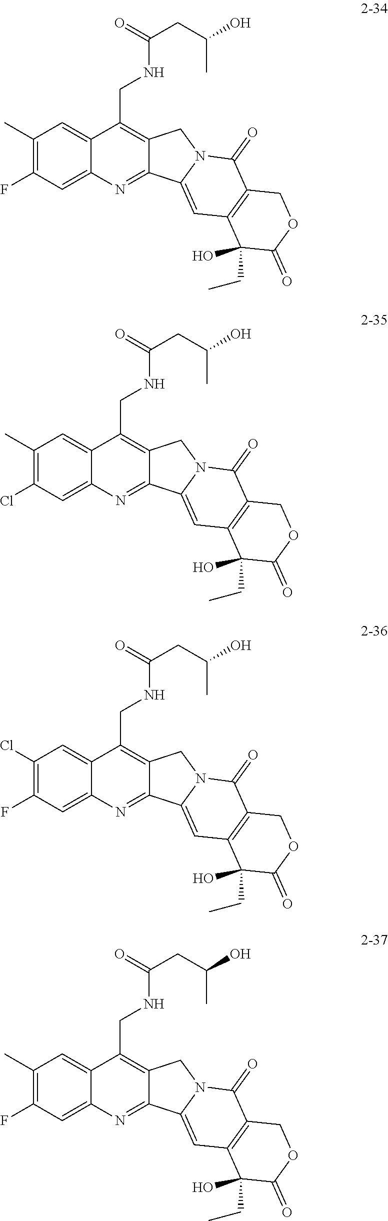 [SMILES: CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O]

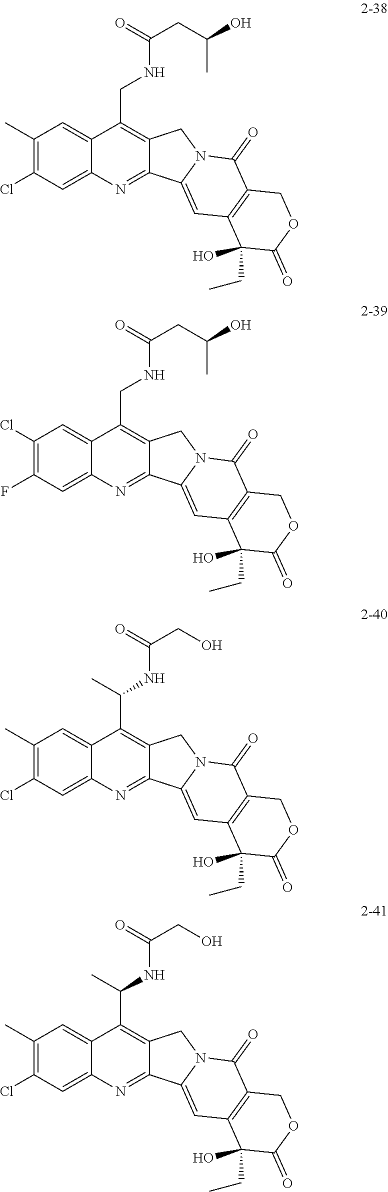

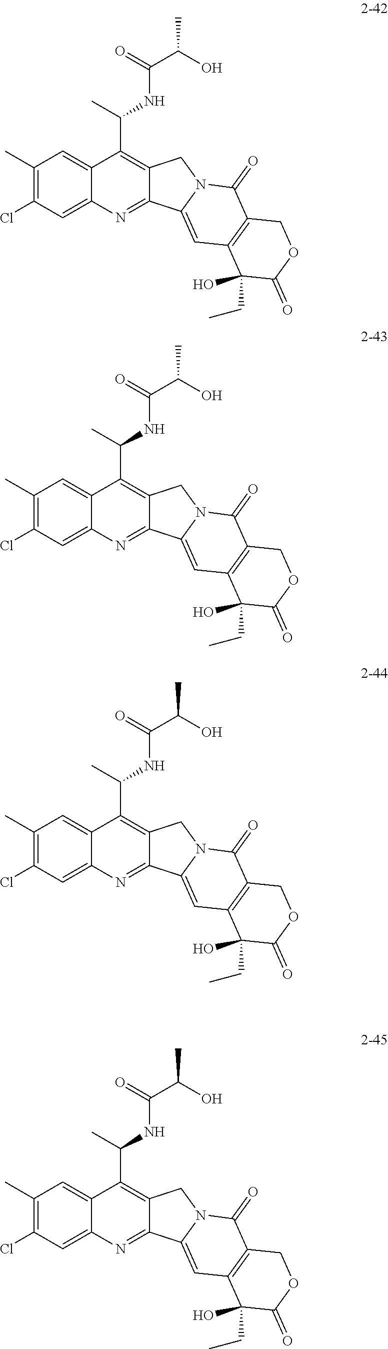

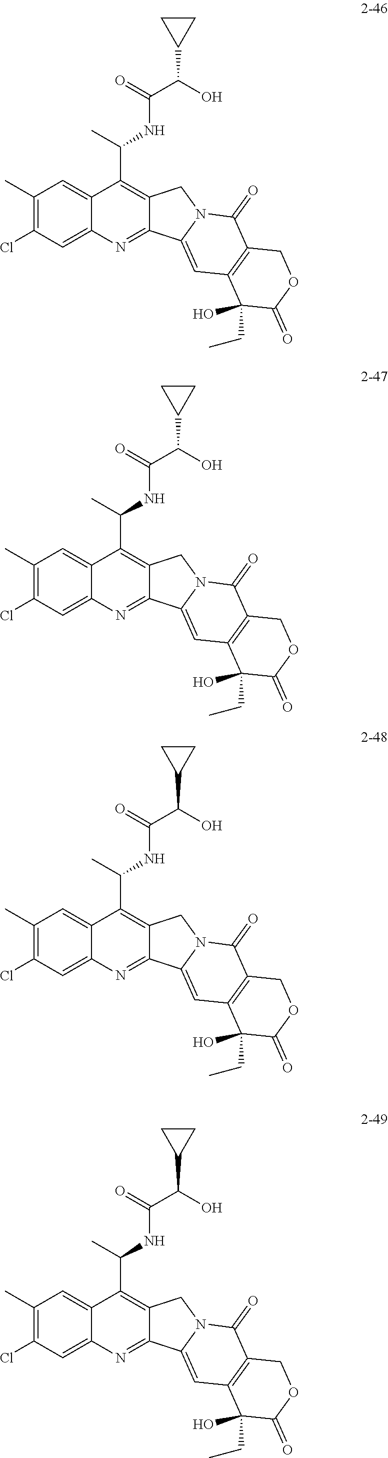

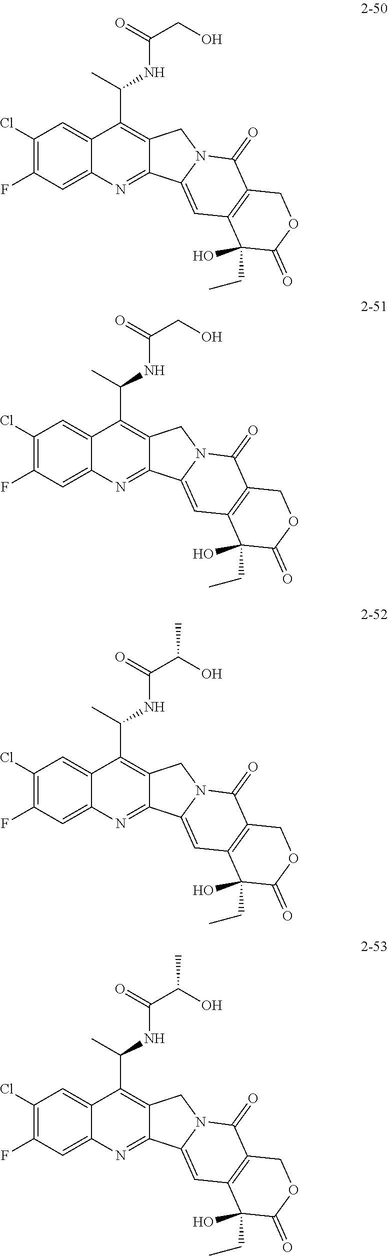

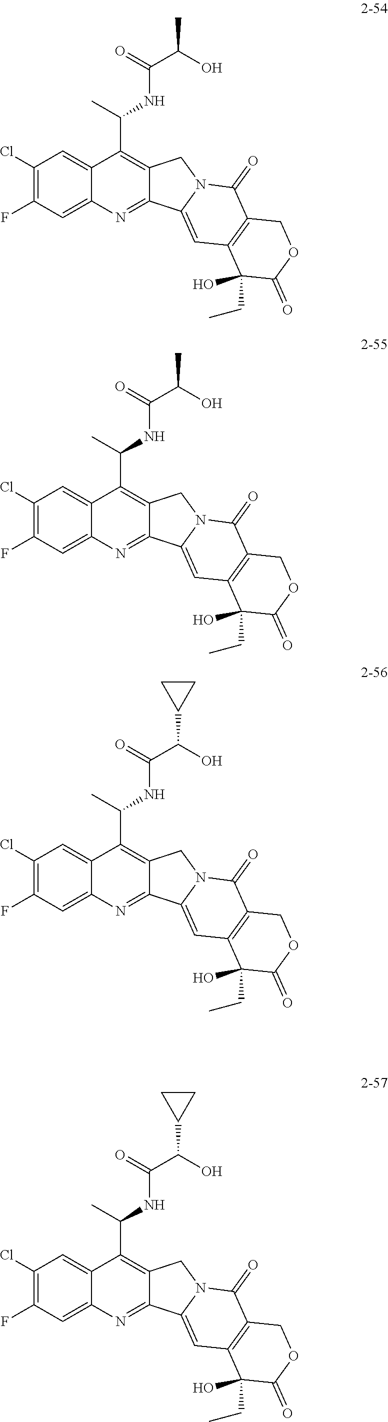

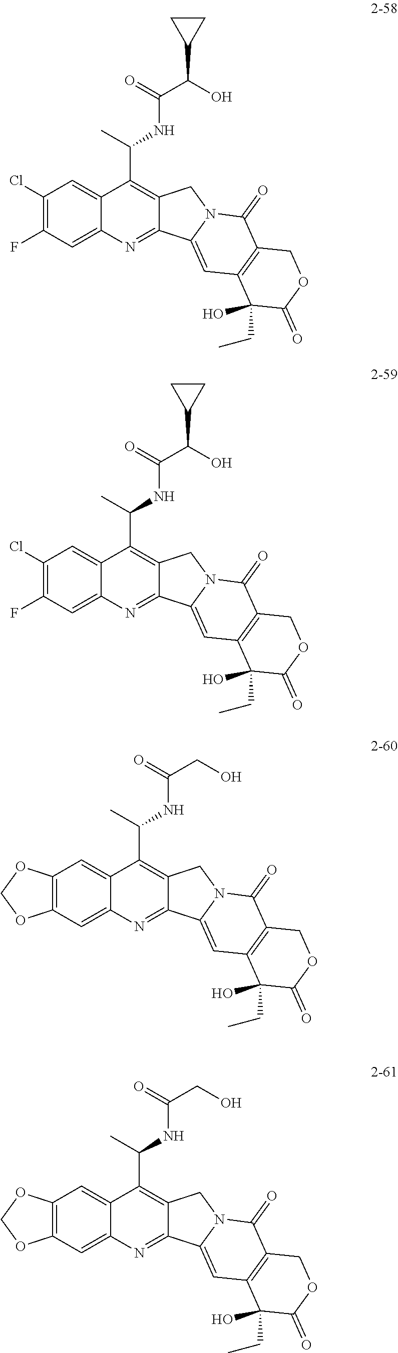

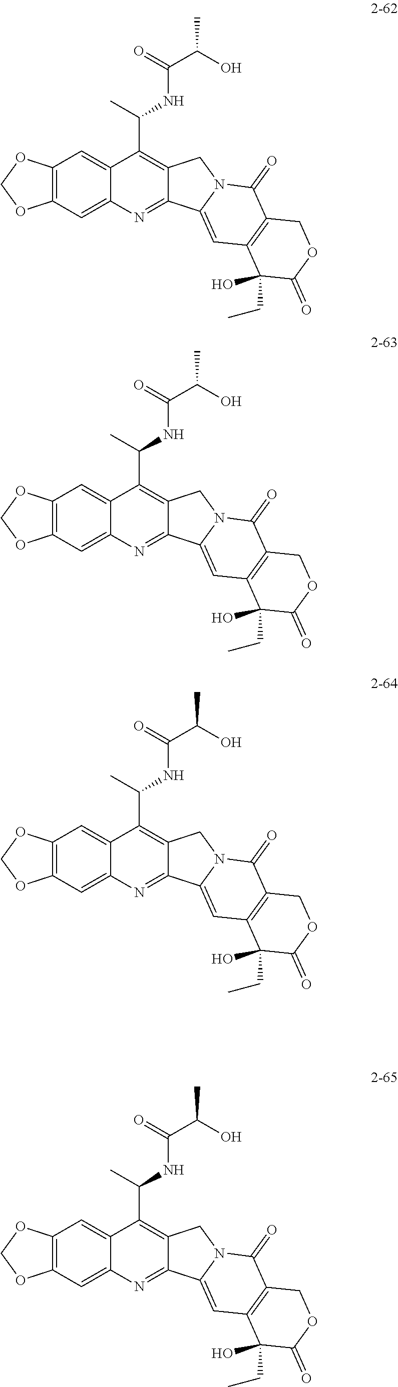

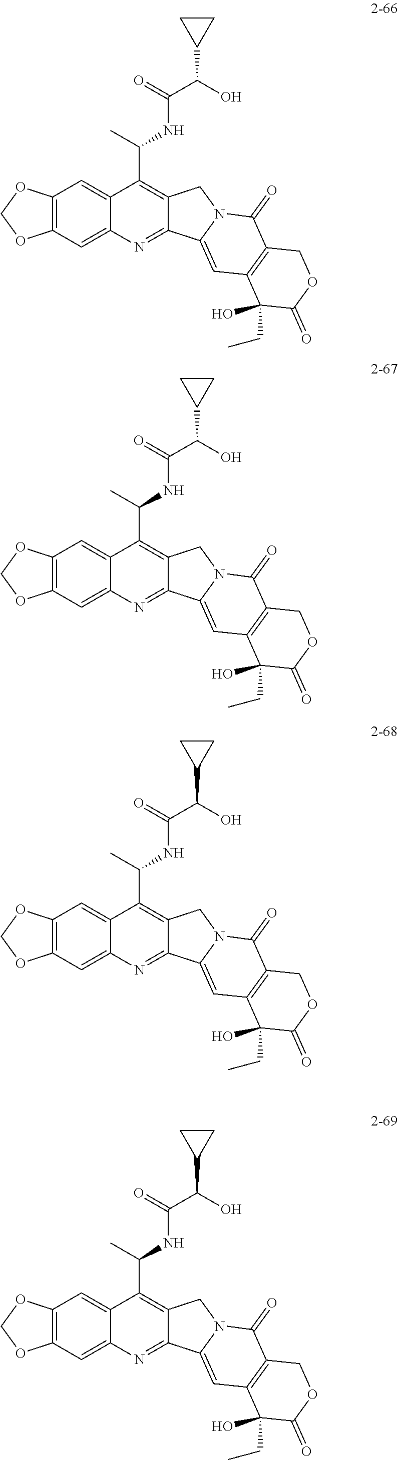

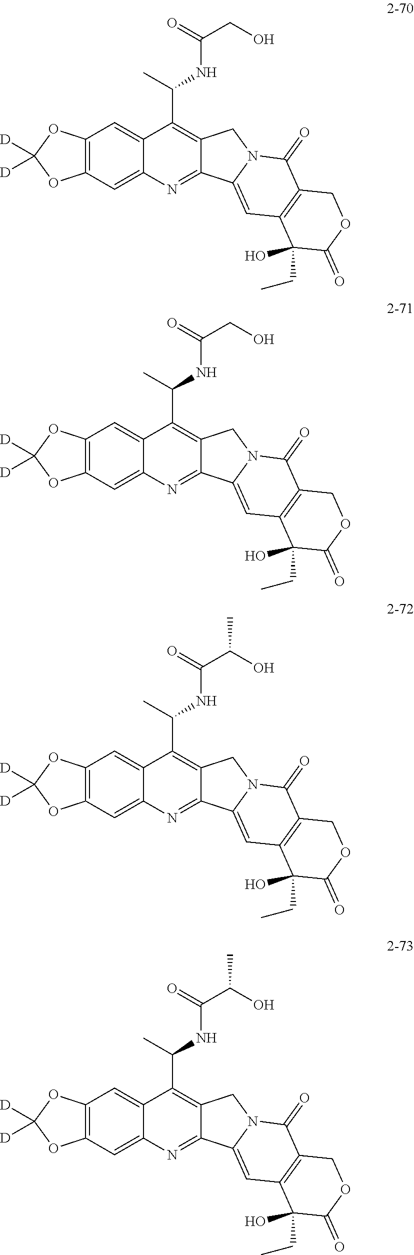

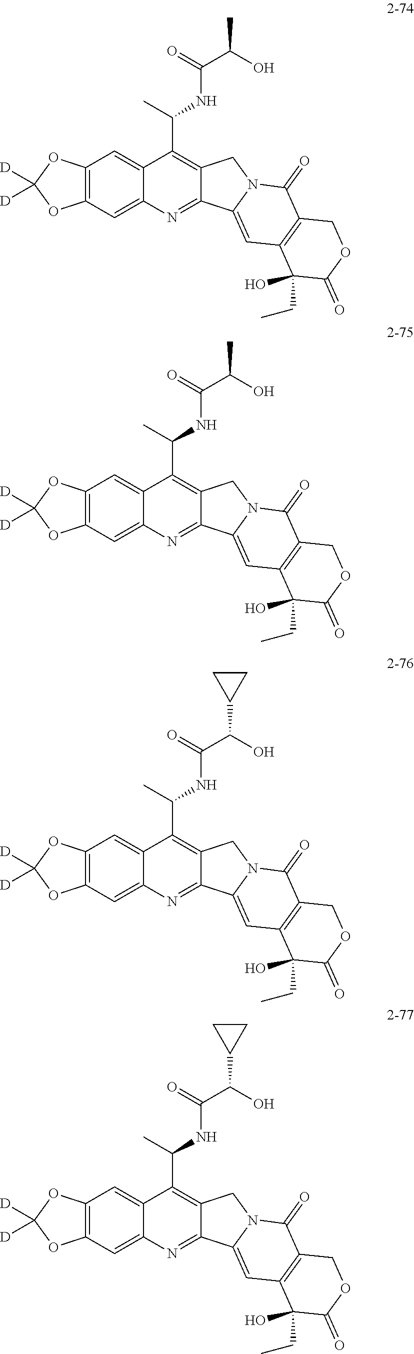

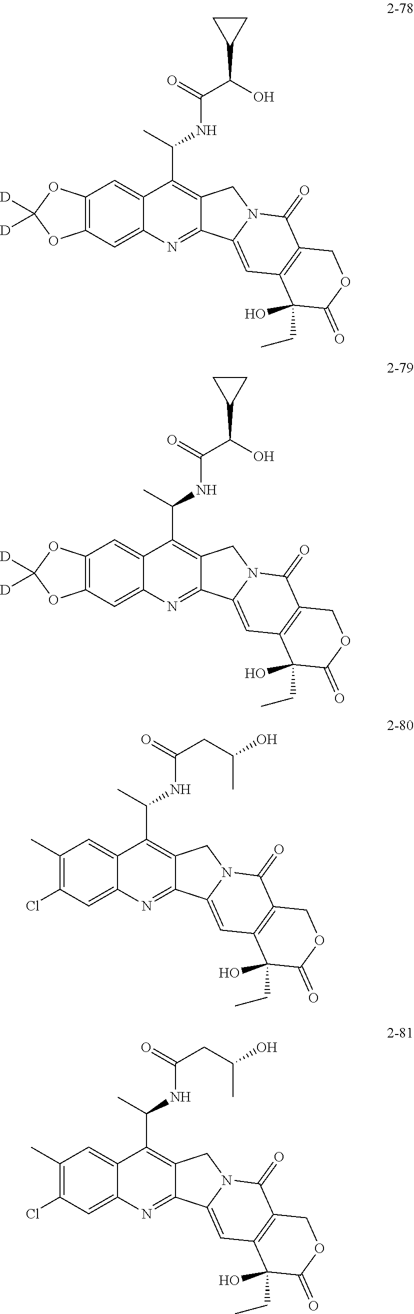

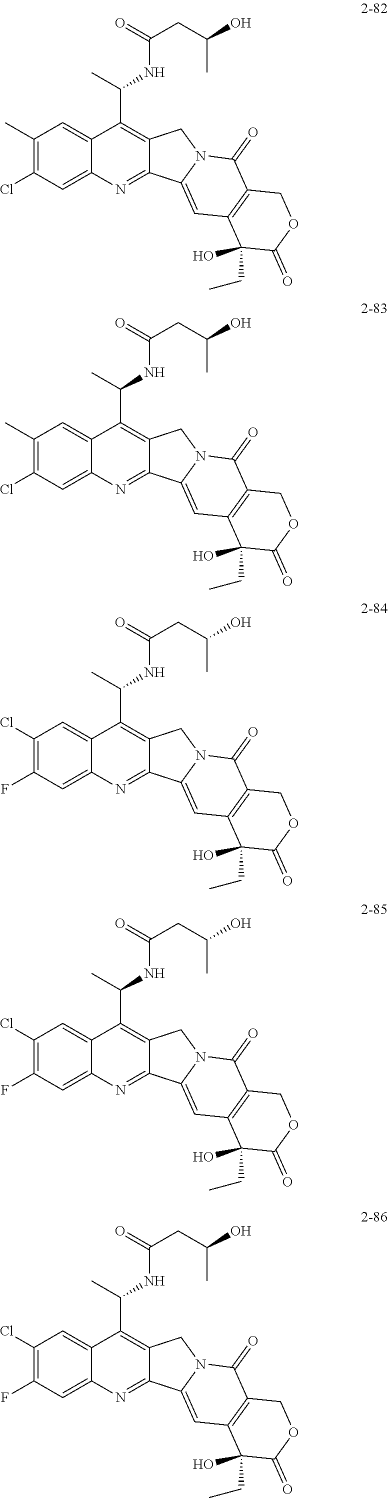

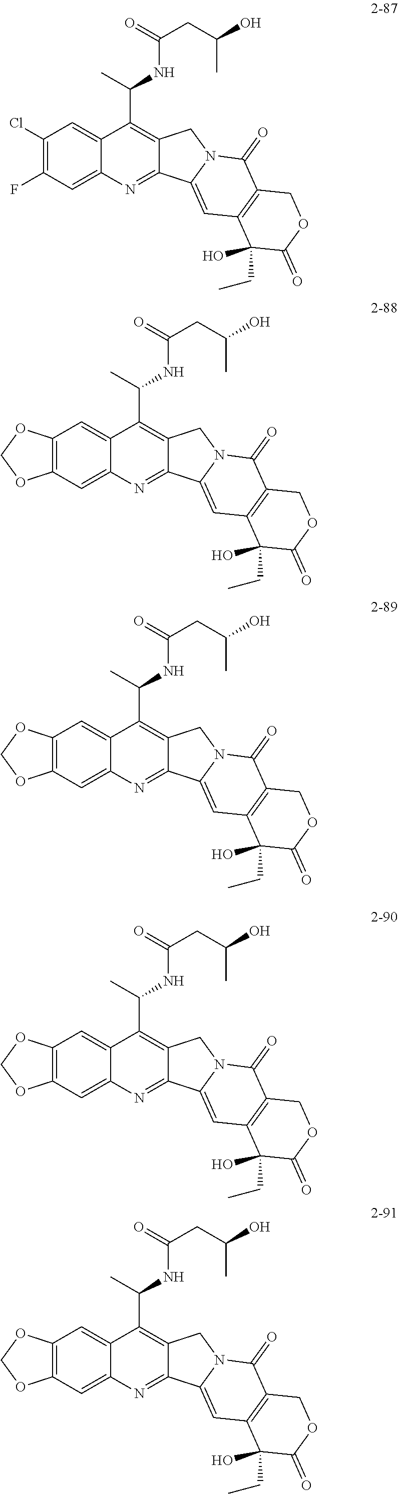

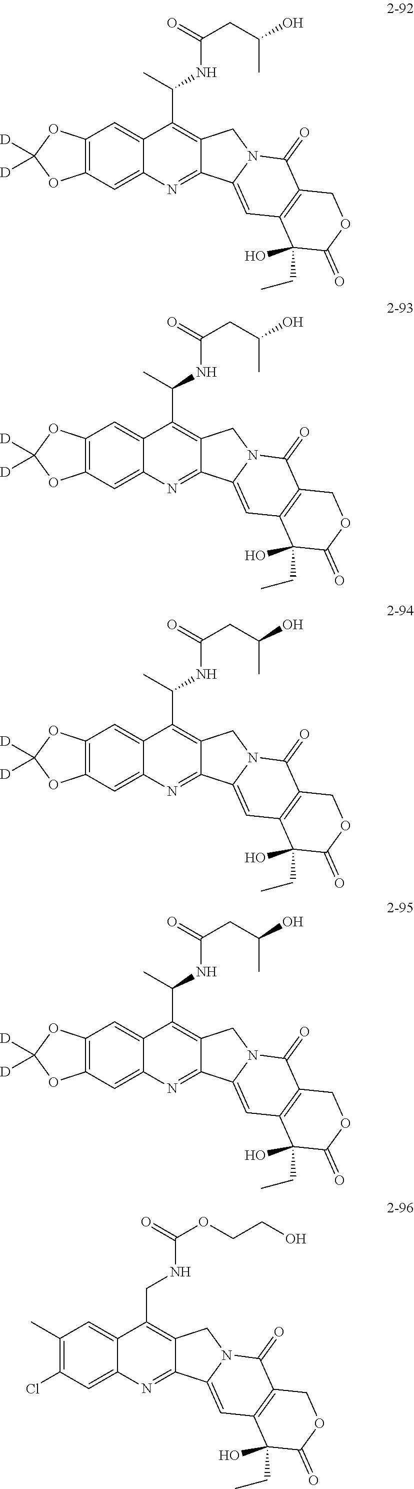

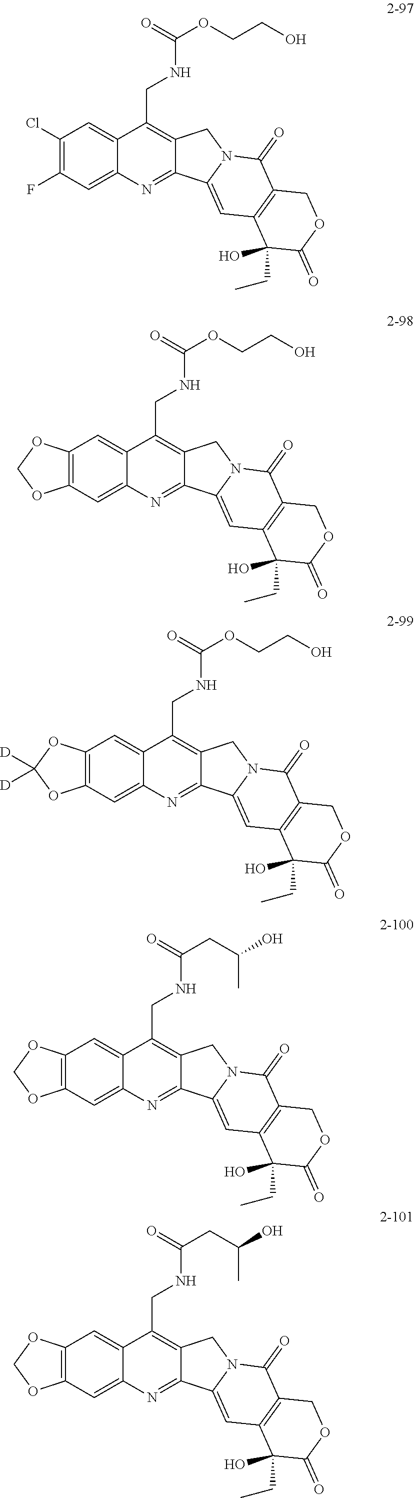

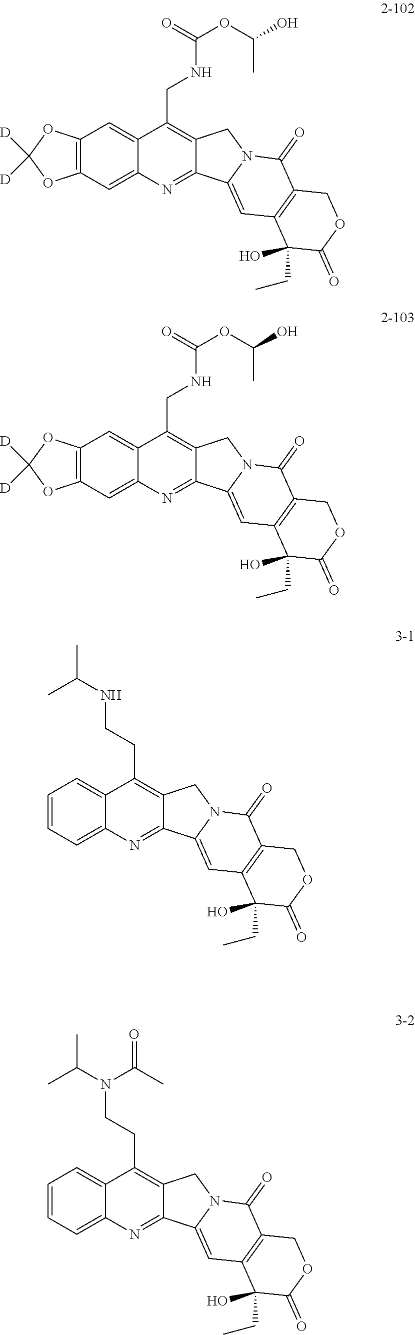 [SMILES: [2H]C1([2H])Oc2cc3nc4c(c(CNC(=O)O[C@@H](C)O)c3cc2O1)Cn1c-4cc2c(c1=O)COC(=O)[C@]2(O)CC ; [2H]C1([2H])Oc2cc3nc4c(c(CNC(=O)O[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C]

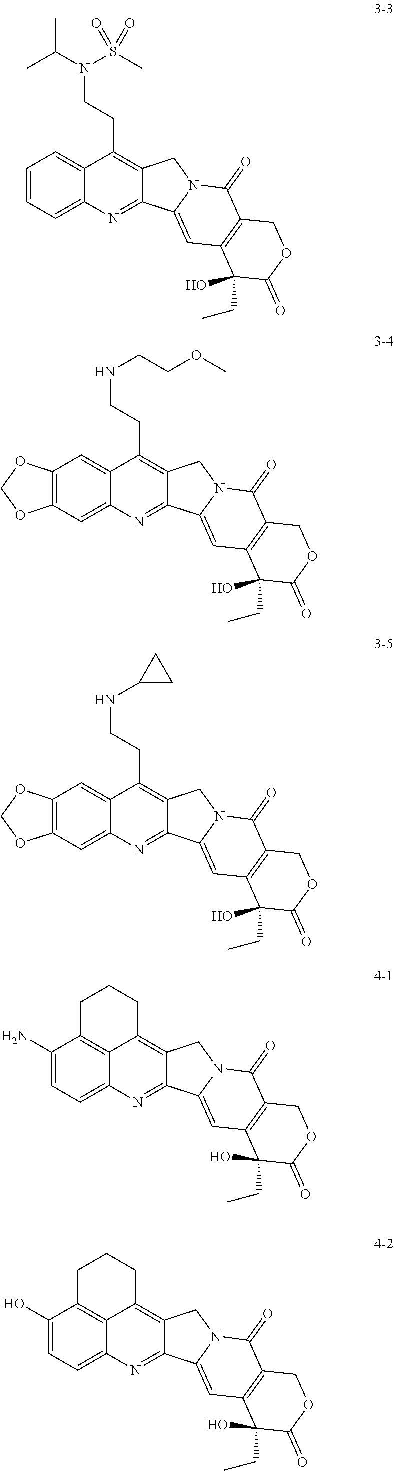

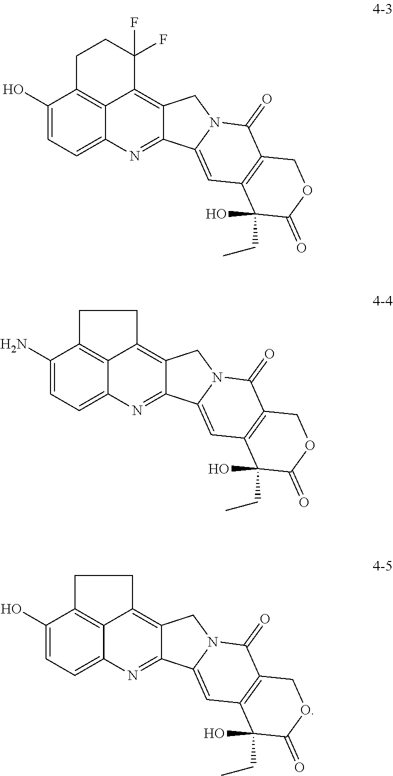 [SMILES: CC[C@@]1(O)C(=O)OCc2c1cc1n(c2=O)Cc2c-1nc1ccc(O)c3c1c2C(F)(F)CC3 ; CC[C@@]1(O)C(=O)OCc2c1cc1n(c2=O)Cc2c-1nc1ccc(N)c3c1c2CC3 ; CC[C@@]1(O)C(=O)OCc2c1cc1n(c2=O)Cc2c-1nc1ccc(O)c3c1c2CC3]

In some embodiments, the cytotoxic drug is selected from the group consisting of the following compounds or isotopically labeled compounds thereof:

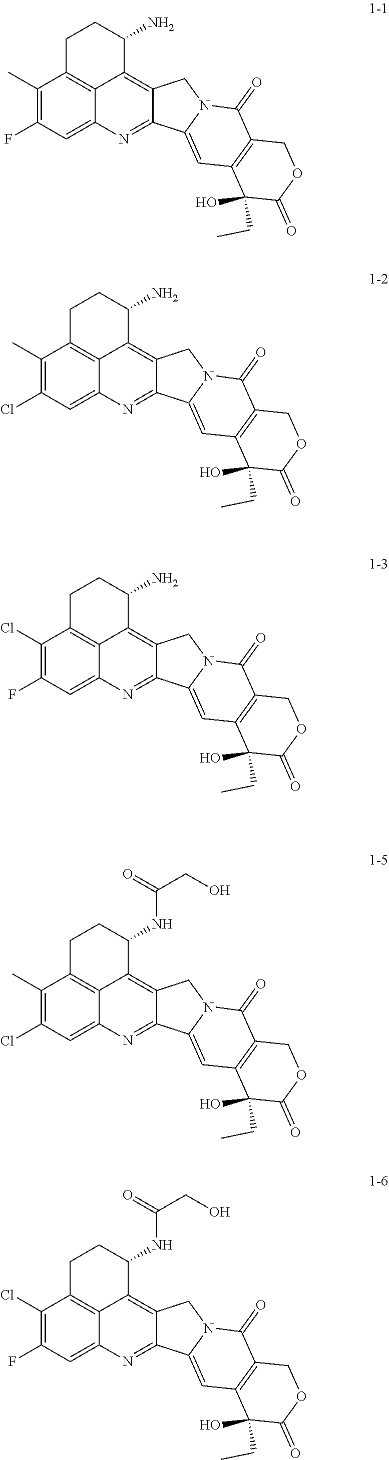 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3]

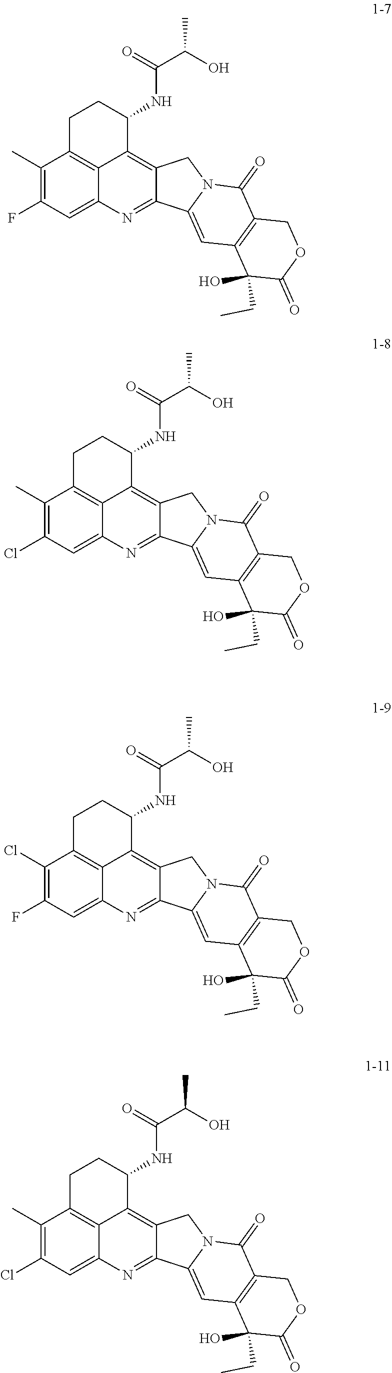 [SMILES: CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3]

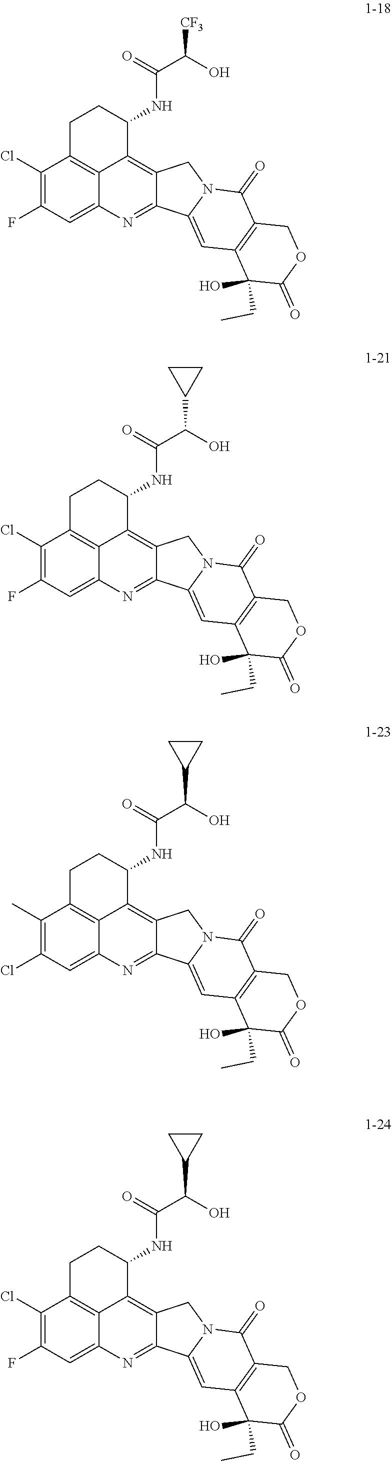 [SMILES: CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3]

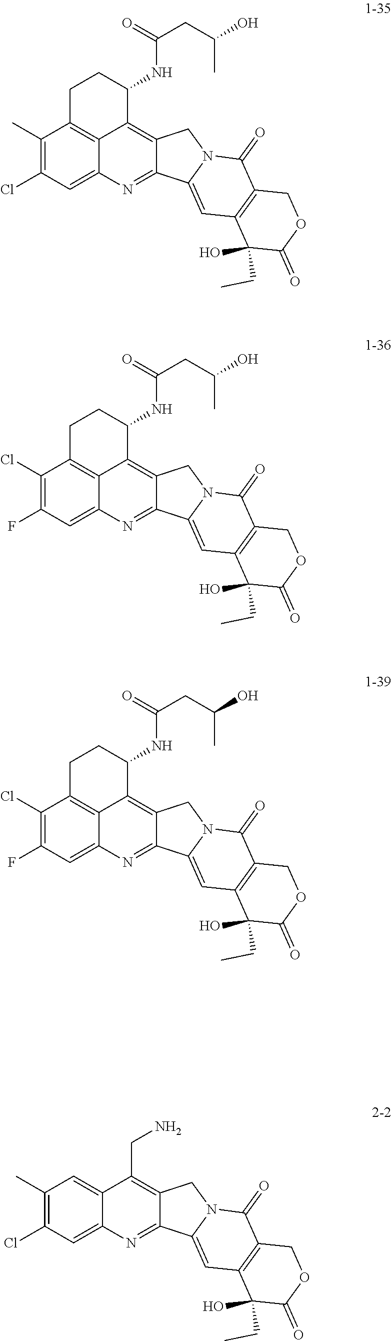 [SMILES: CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3 ; CC[C@@]1(O)C(=O)OCc2c1cc1n(c2=O)Cc2c-1nc1cc(Cl)c(C)cc1c2CN]

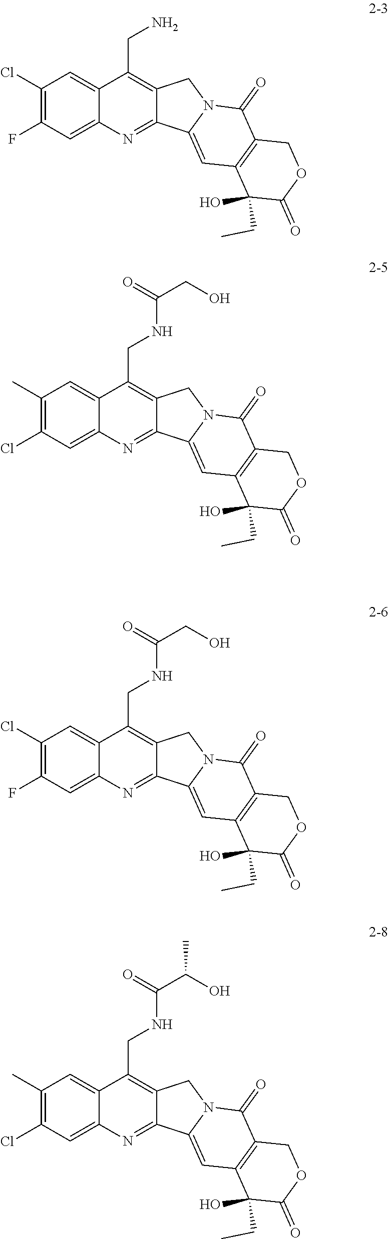 [SMILES: CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(Cl)c(C)cc1c2CNC(=O)[C@H](C)O]

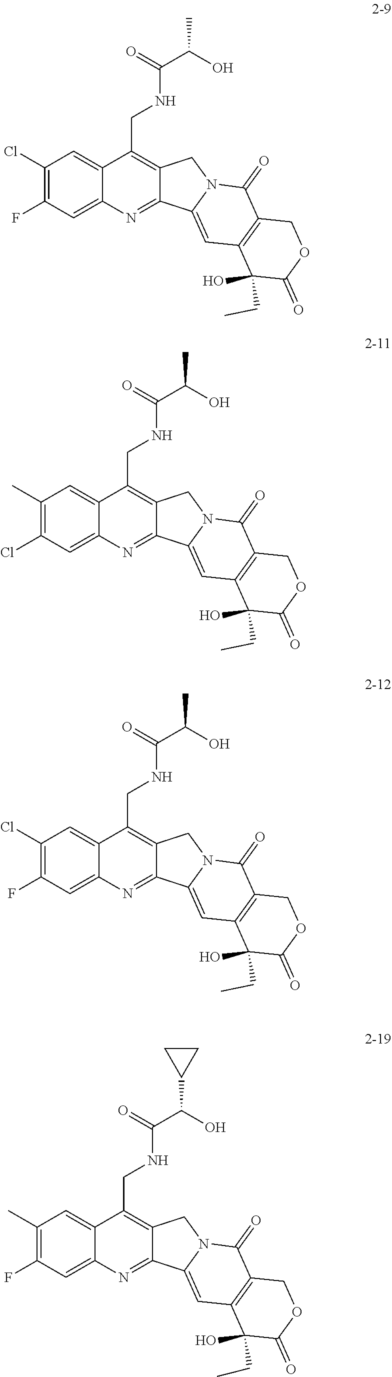 [SMILES: CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](O)C1CC1]

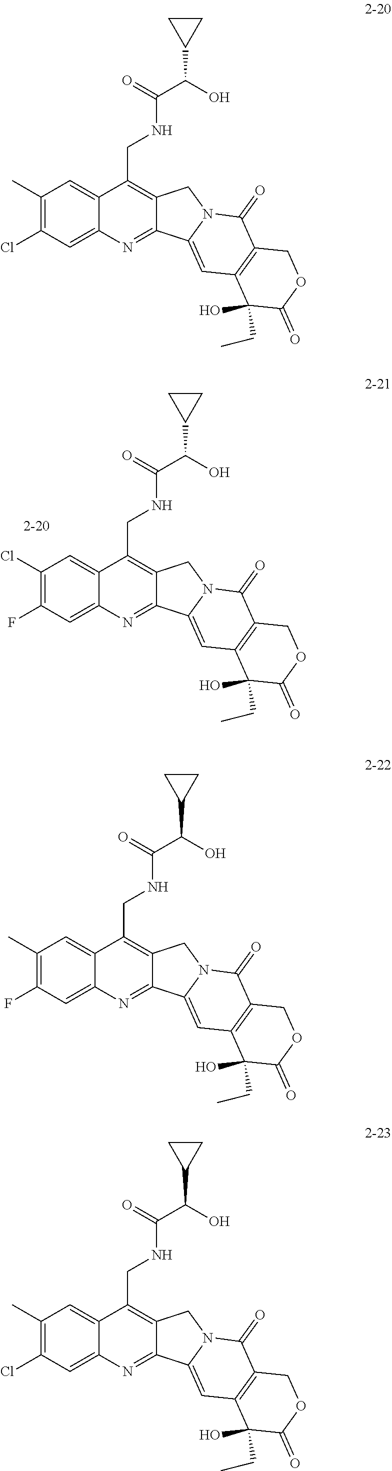

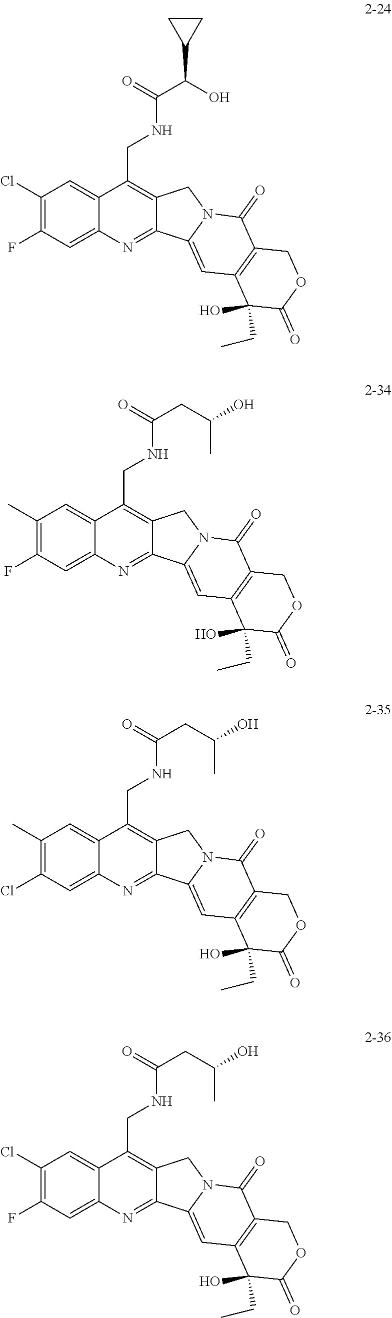 [SMILES: CC[C@@]1(O)C(=O)OCc2c1cc1n(c2=O)Cc2c-1nc1cc(F)c(Cl)cc1c2CNC(=O)[C@H](O)C1CC1 ; CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O]

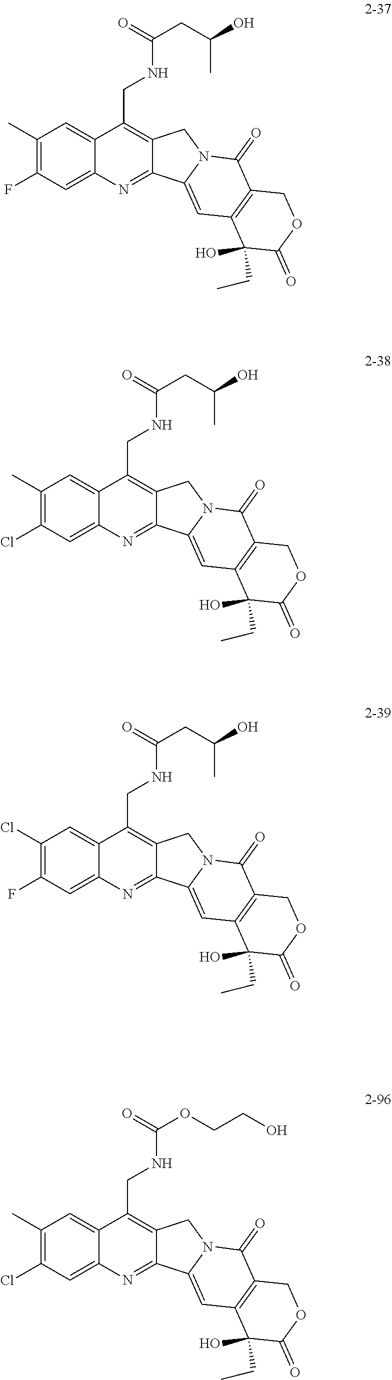 [SMILES: CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2CNC(=O)OCCO]

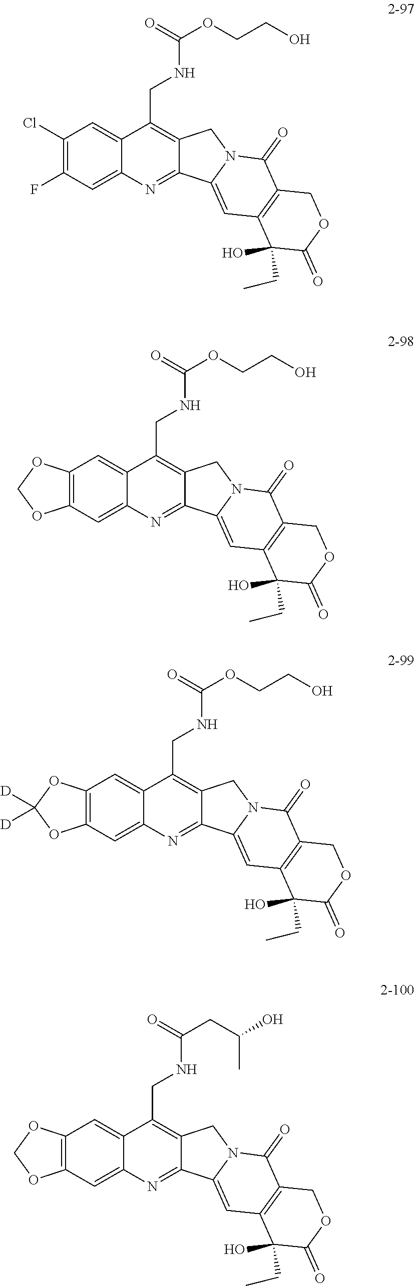 [SMILES: CC[C@@]1(O)C(=O)OCc2c1cc1n(c2=O)Cc2c-1nc1cc(F)c(Cl)cc1c2CNC(=O)OCCO ; CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC ; CC[C@@]1(O)C(=O)OCc2c1cc1n(c2=O)Cc2c-1nc1cc3c(cc1c2CNC(=O)C[C@@H](C)O)OCO3]

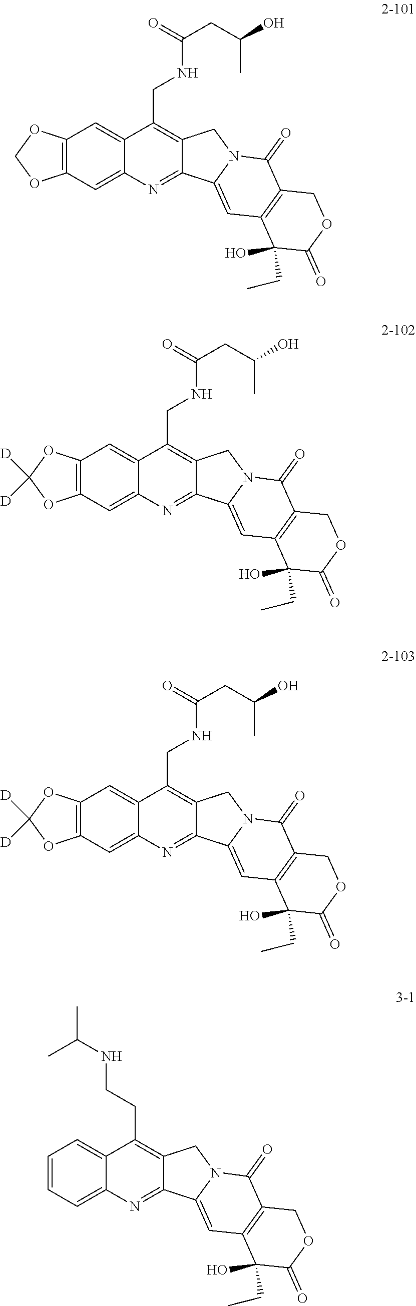 [SMILES: CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C]

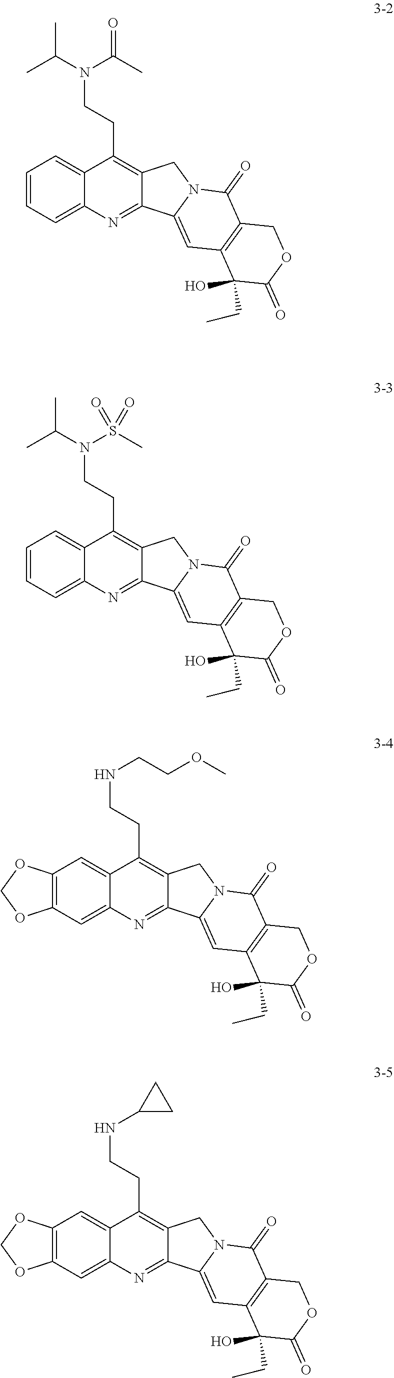 [SMILES: CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1cc3c(cc1c2CCNC1CC1)OCO3]

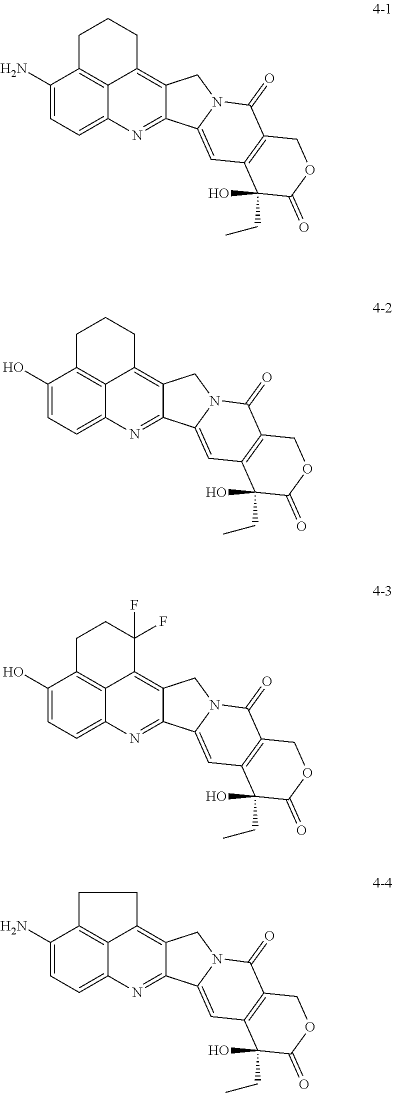 [SMILES: CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2C(F)(F)CC3 ; CC[C@@]1(O)C(=O)OCc2c1cc1n(c2=O)Cc2c-1nc1ccc(N)c3c1c2CC3]

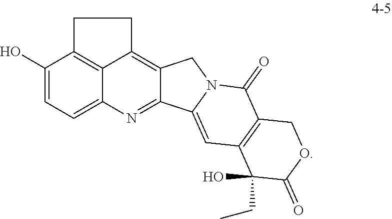 [SMILES: CC[C@@]1(O)C(=O)OCc2c1cc1n(c2=O)Cc2c-1nc1ccc(O)c3c1c2CC3]

In some embodiments, the cytotoxic drug is selected from the group consisting of the following compounds or isotopically labeled compounds thereof:

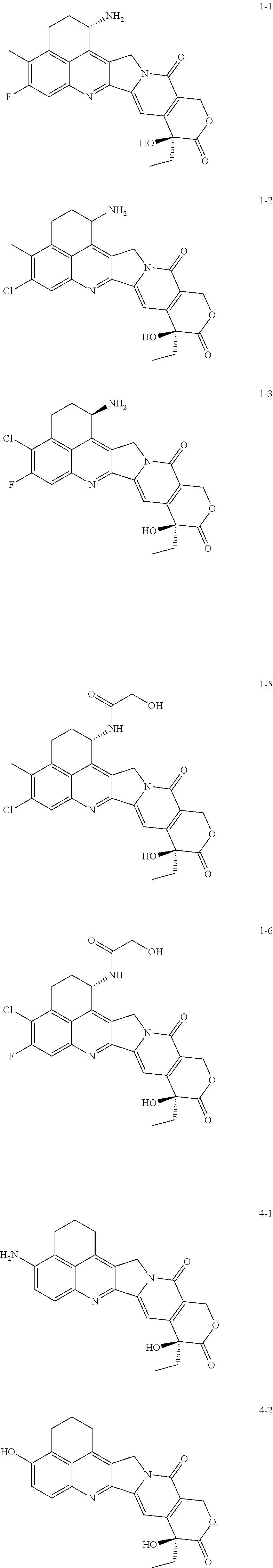

In some embodiments, the cytotoxic drug is selected from the group consisting of the following compounds or isotopically labeled compounds thereof:

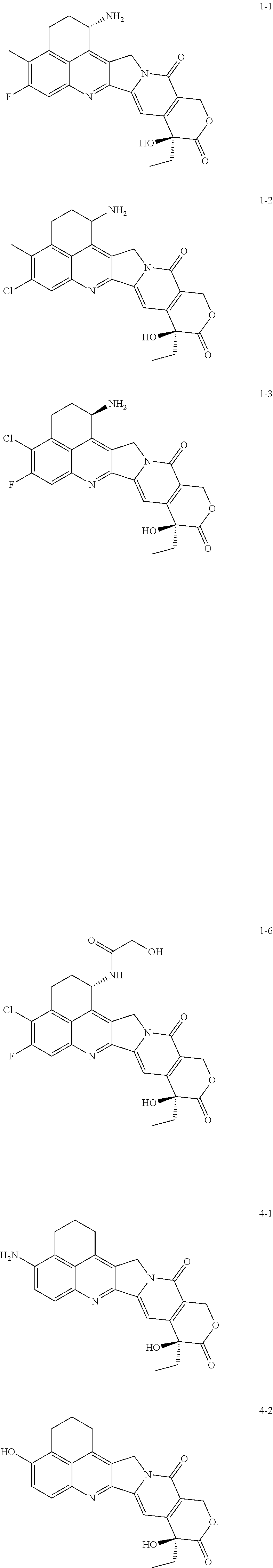 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3]

In some embodiments, the cytotoxic drug is selected from the group consisting of the following compounds or isotopically labeled compounds thereof:

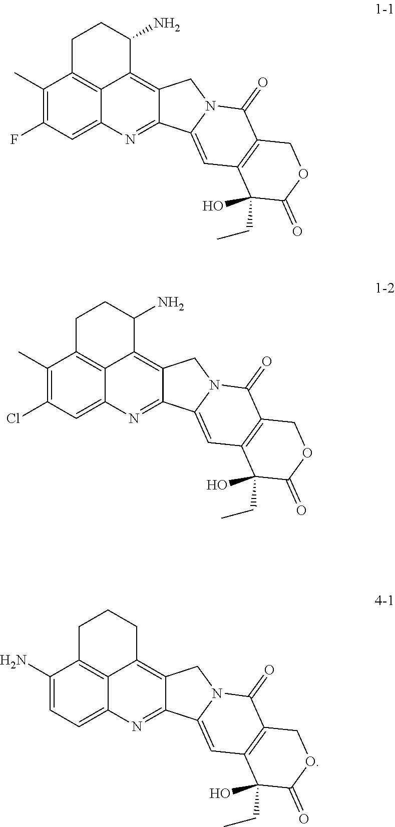 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1ccc(N)c3c1c2CCC3]

In some embodiments, the cytotoxic drug is selected from the group consisting of the following compound or isotopically labeled compounds thereof:

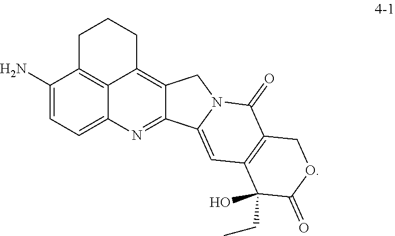 [SMILES: CC[C@@]1(O)C(=O)OCc2c1cc1n(c2=O)Cc2c-1nc1ccc(N)c3c1c2CCC3]

In some embodiments, the cytotoxic drug is selected from the group consisting of the following Formula III and Formula IV

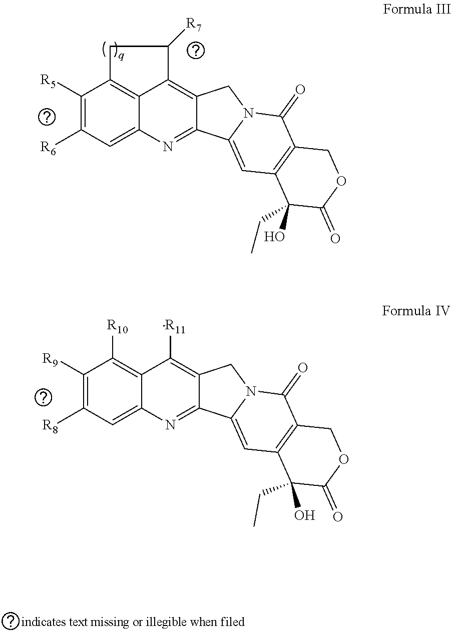

wherein, R₅, R₆ are each independently selected from the group consisting of OH, —NH₂, —NH(C₁₋₆ alkyl), C₁₋₆ alkyl and halogen; the C₁₋₆ alkyl is optionally further substituted with one or more substituents selected from the group consisting of halogen, hydroxyl, C₁₋₆ haloalkyl, C₃₋₆ cycloalkyl;
R₇ is selected from the group consisting of H, —OH, —NH₂, —NH(C₁₋₆ alkyl), and —NH—CO—(C₁₋₆ alkylene)-OH; the C₁₋₆ alkyl is optionally further substituted with one or more substituents selected from the group consisting of halogen, hydroxyl, C₁₋₆ haloalkyl, C₃₋₆ cycloalkyl;
q is 1 or 2;
R₈ and R₉ are each independently selected from the group consisting of H, halogen and hydroxyl; or R₄ and R₅ are connected to the carbon atom connected thereto to form a 5- to 6-membered oxygen-containing heterocycle; the 5- to 6-membered oxygen-containing heterocycle is optionally substituted with one or more substituents selected from the group consisting of deuterium (D), halogen, hydroxyl, C₁₋₆ haloalkyl, C₃₋₆ cycloalkyl.
R₁₀ is selected from the group consisting of hydrogen or —C₁₋₄ alkylene-NRₐR_{b};
R₁₁ is selected from the group consisting of C₁₋₆ alkyl and —C₁₋₄ alkylene-NRₐR_{b}; and
wherein Rₐ, R_{b} are each independently selected from the group consisting of H, C₁₋₆ alkyl, C₃₋₆ cycloalkyl, —SO₂—C₁₋₆ alkyl and —CO—C₁₋₆ alkyl at each occurrence; the C₁₋₆ alkyl and C₁₋₄ alkylene are optionally further substituted with one or more substituents selected from the group consisting of halogen, hydroxyl, C₁₋₆ haloalkyl, C₃₋₆ cycloalkyl.

In some embodiments, R₅, R₆ are each independently selected from the group consisting of OH, —NH₂, C₁₋₄ alkyl and halogen; the C₁₋₄ alkyl is optionally further substituted with one or more substituents selected from the group consisting of halogen, hydroxyl, C₁₋₄ haloalkyl, C₃₋₆ cycloalkyl.

In some embodiments, R₇ is selected from the group consisting of —H, —NH₂, —NH(C₁₋₄ alkyl), and —NH—CO—(C₁₋₄ alkylene)-OH; the C₁₋₄ alkyl is optionally further substituted with one or more substituents selected from the group consisting of halogen, hydroxyl, C₁₋₄ haloalkyl, and C₃₋₆ cycloalkyl.

In some embodiments, Rₐ, R_{b} are independently selected from the group consisting of H, C₁₋₄ alkyl, C₃₋₆ cycloalkyl, —SO₂—C₁₋₄ alkyl, and —CO—C₁₋₄ alkyl at each occurrence; the C₁₋₄ alkyl is optionally further substituted with one or more substituents selected from the group consisting of halogen, hydroxyl, C₁₋₆ haloalkyl, and C₃₋₆ cycloalkyl.

In some embodiments, R₅, R₆ are independently selected from the group consisting of OH, —NH₂, C₁₋₆ alkyl, and halogen;
R₇ is selected from the group consisting of —H, —NH₂, —NH(C₁₋₆ alkyl), and —NH—CO—(C₁₋₆ alkylene)-OH; and
q is 2.

In some embodiments, R₅, R₆ are each independently selected from the group consisting of OH, —NH₂, C₁₋₄ alkyl and halogen. In some embodiments, R₅, R₆ are each independently selected from the group consisting of OH, —NH₂, methyl, ethyl, N-propyl, isopropyl and halogen. In some embodiments, R₅, R₆ are each independently selected from the group consisting of OH, —NH₂, methyl, fluorine and chlorine.

In some embodiments, R₇ is selected from the group consisting of —H, —NH₂, —NH(C₁₋₄ alkyl), and —NH—CO—(C₁₋₄ alkylene)-OH. In some embodiments, R₇ is selected from the group consisting of —H, —NH₂, —NH(CH₃), —NH(CH₂CH₃), —NH(CH(CH₃)₂), —NH—CO—CH₂—OH, —NH—CO—CH₂CH₂—OH, —NH—CO—CH₂(CH₃)—OH. In some embodiments, R_{7A} is selected from the group consisting of —H, —NH₂, and —NH—CO—CH₂—OH.

In some embodiments, the cytotoxic drug fragment D has a structure represented by Formula III′:

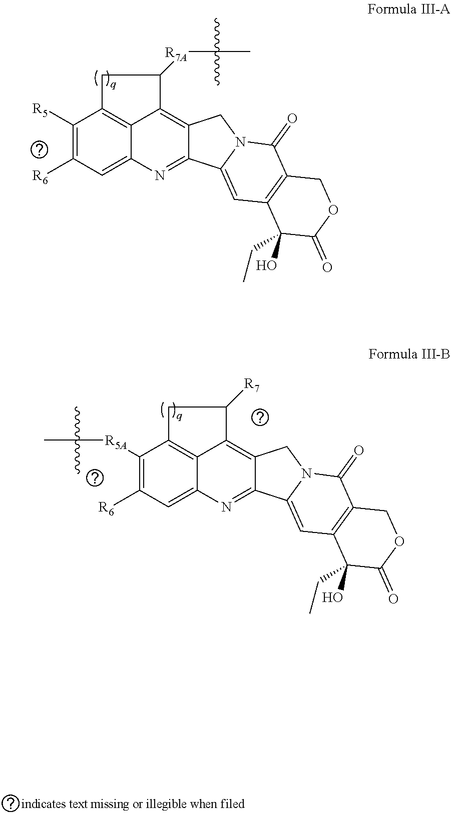

wherein, R_{5A} is selected from the group consisting of —O— or —N—; R_{7A} is selected from the group consisting of a chemical bond, —O—, —NH—, —N(C₁₋₆ alkyl)-, and —NH—CO—(C₁₋₆ alkylene)-O—; the C₁₋₆ alkyl is optionally further substituted with one or more substituents selected from the group consisting of halogen, hydroxyl, C₁₋₆ haloalkyl, and C₃₋₆ cycloalkyl;
R₅ to R₇, q are as described above.

In some embodiments, R_{7A} is selected from the group consisting of a chemical bond, —O—, —NH—, —N(C₁₋₄ alkyl)-, and —NH—CO—(C₁₋₄ alkylene)-O—; the C₁₋₄ alkyl is optionally further substituted with one or more substituents selected from the group consisting of halogen, hydroxyl, C₁₋₄ haloalkyl, and C₃₋₆ cycloalkyl. In some embodiments, R_{7A} is selected from the group consisting of a chemical bond, —O—, —NH—, —N(CH₃)—, —N(CH₂CH₃)—, —N(CH(CH₃)₂)—, —NH—CO—CH₂—O—, —NH—CO—CH₂CH₂—O—, —NH—CO—CH₂(CH₃)—O—. In some embodiments, R_{7A} is selected from the group consisting of —NH—, and —NH—CO—CH₂—O—.

In some embodiments, D is selected from the group consisting of the following structures:

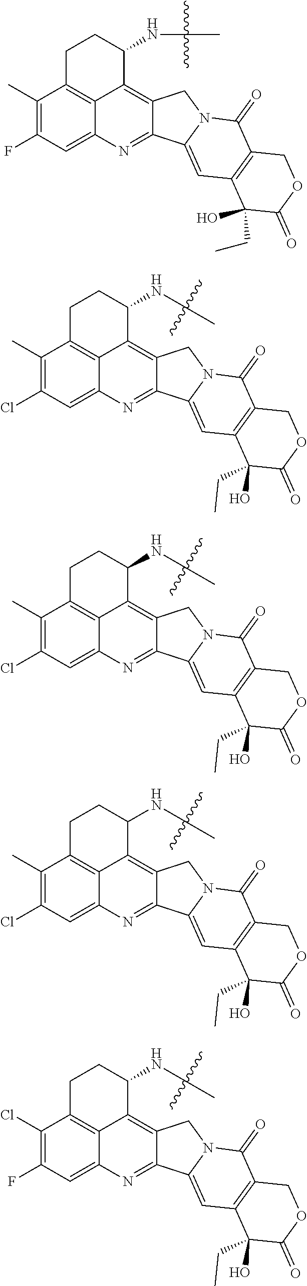 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC ; *N[C@H]1CCc2c(Cl)c(F)cc3nc4c(c1c23)Cn1c-4cc2c(c1=O)COC(=O)[C@]2(O)CC]

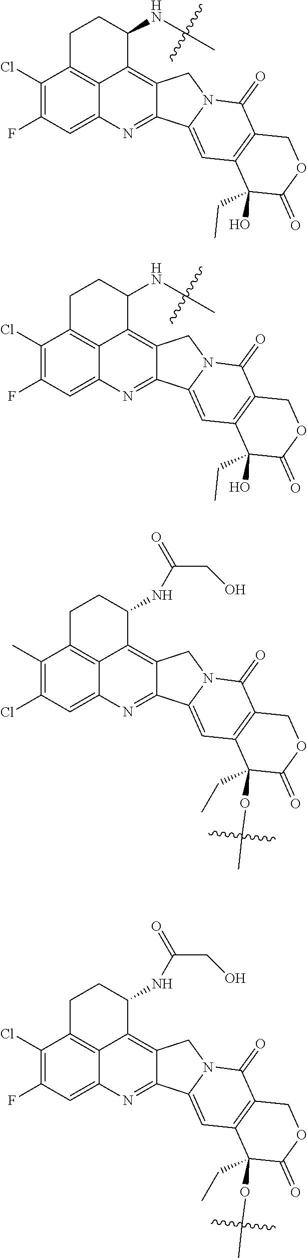 [SMILES: *N[C@@H]1CCc2c(Cl)c(F)cc3nc4c(c1c23)Cn1c-4cc2c(c1=O)COC(=O)[C@]2(O)CC ; *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3]

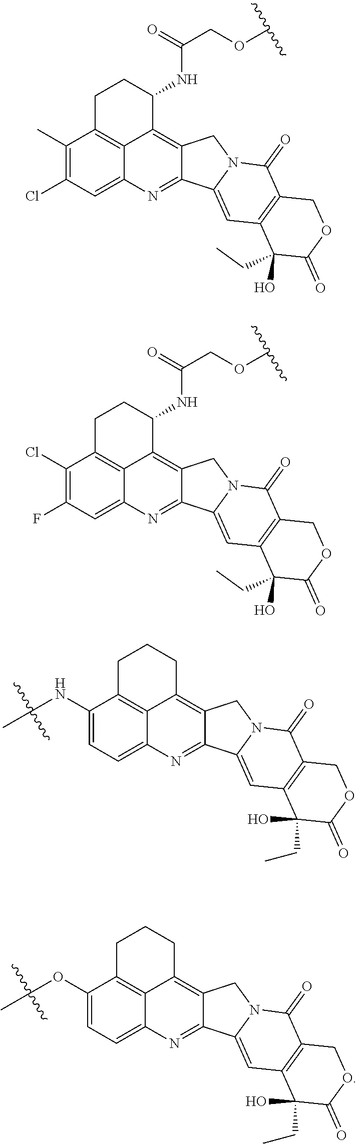 [SMILES: *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC ; *Nc1ccc2nc3c(c4c2c1CCC4)Cn1c-3cc2c(c1=O)COC(=O)[C@]2(O)CC ; *Oc1ccc2nc3c(c4c2c1CCC4)Cn1c-3cc2c(c1=O)COC(=O)[C@]2(O)CC]

In some embodiments, the cytotoxic drug is selected from the group consisting of the following compounds or isotopically labeled compounds thereof: 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 2-1, 2-2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 2-80, 2-81, 2-82, 2-83, 2-84, 2-85, 2-86, 2-87, 2-88, 2-89, 2-90, 2-91, 2-92, 2-93, 2-94, 2-95, 2-96, 2-97, 2-98, 2-99, 2-100, 2-101, 2-102, 2-103, 3-1, 3-2, 3-3, 3-4, 3-5.

In some embodiments, the cytotoxic drug is selected from the group consisting of the following compounds or isotopically labeled compounds thereof:

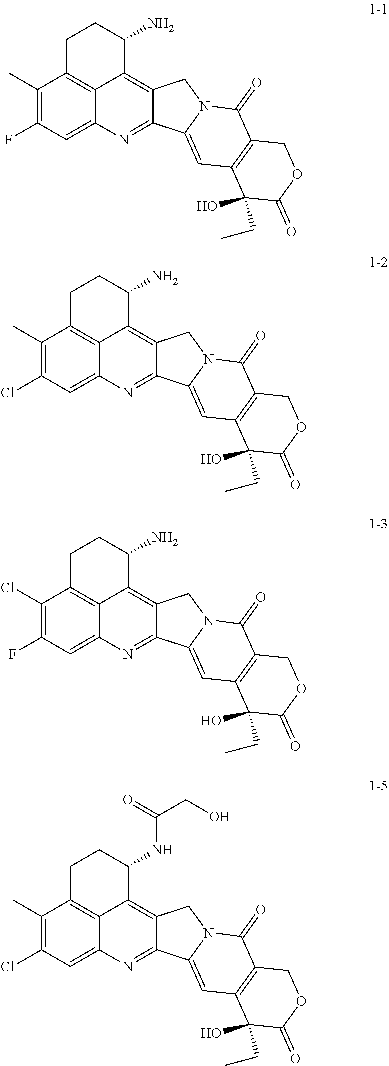 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3]

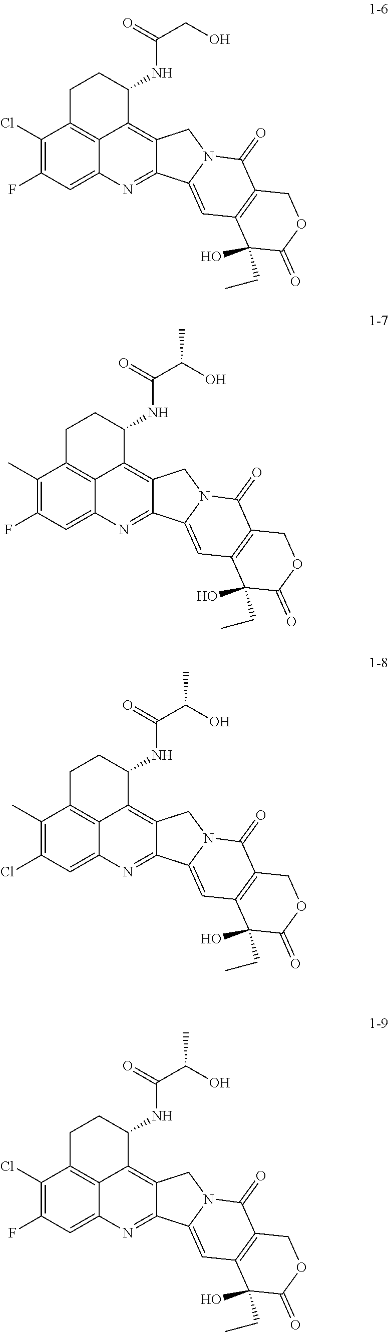 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3]

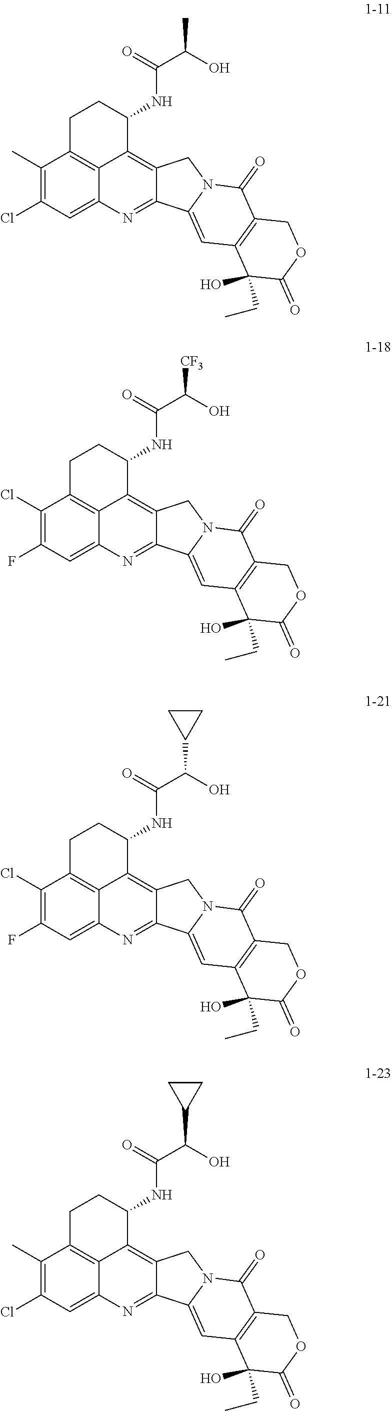 [SMILES: CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3]

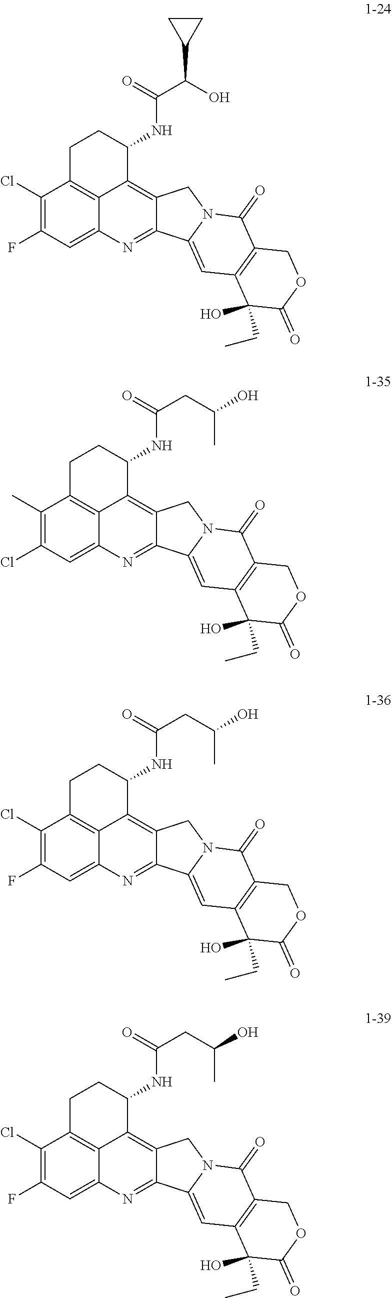 [SMILES: CC[C@@]1(O)C(=O)OCc2c1cc1n(c2=O)Cc2c-1nc1cc(F)c(Cl)c3c1c2[C@@H](NC(=O)[C@H](O)C1CC1)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3]

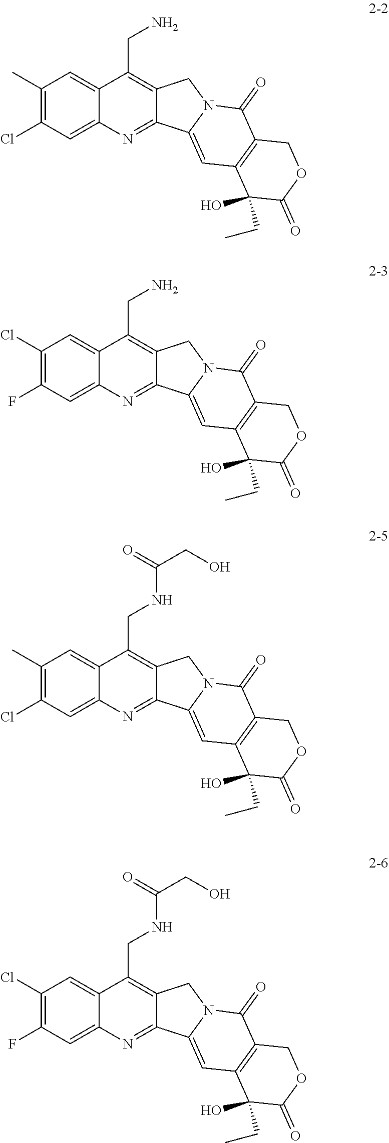 [SMILES: CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO]

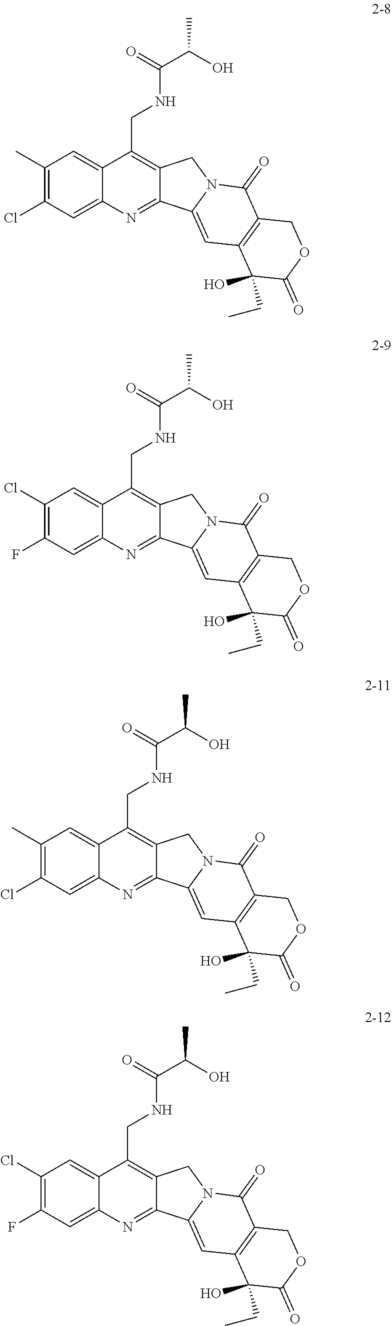 [SMILES: CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O]

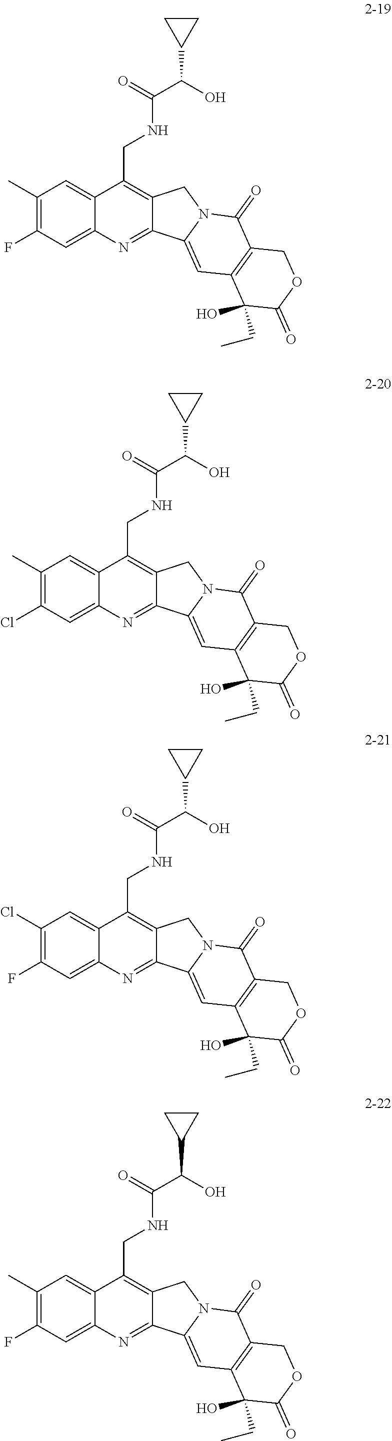

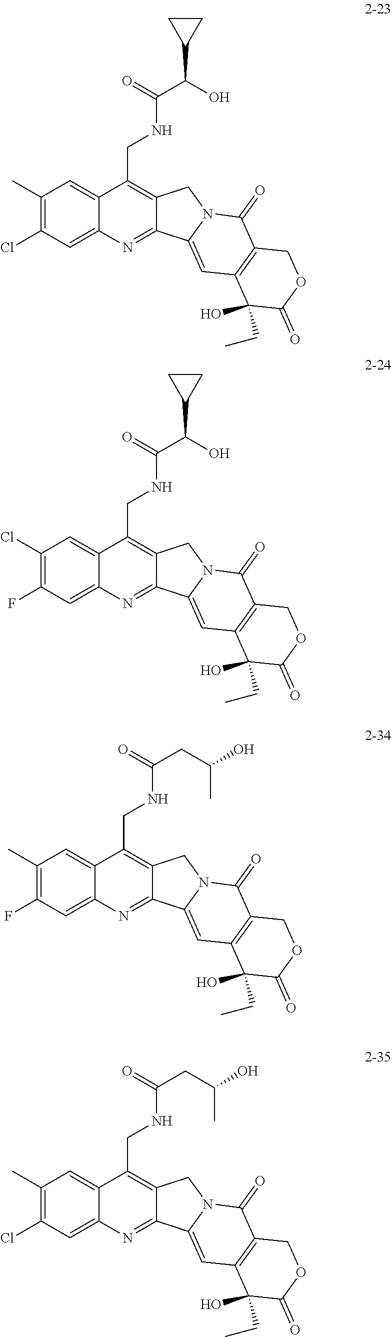

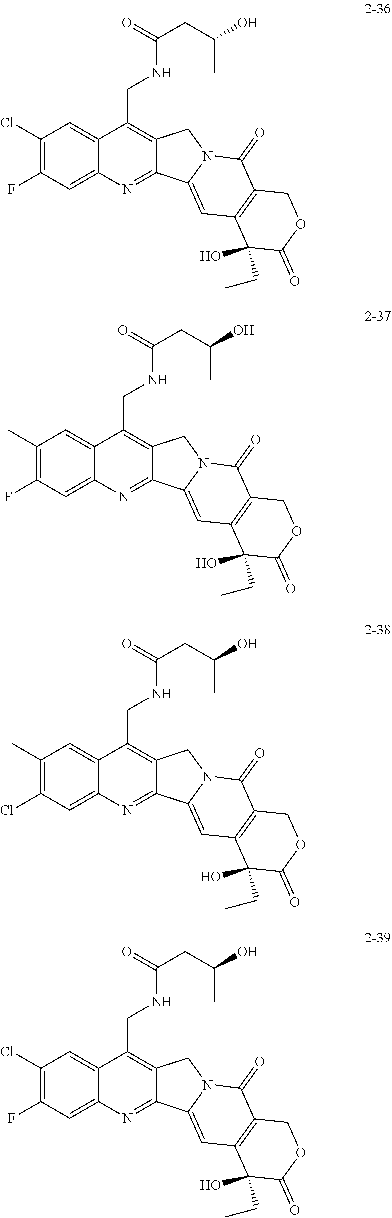

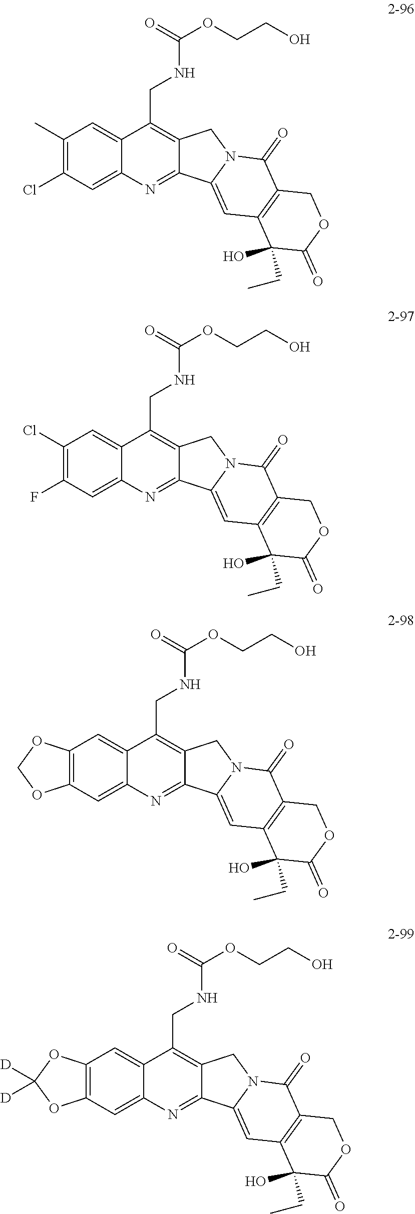

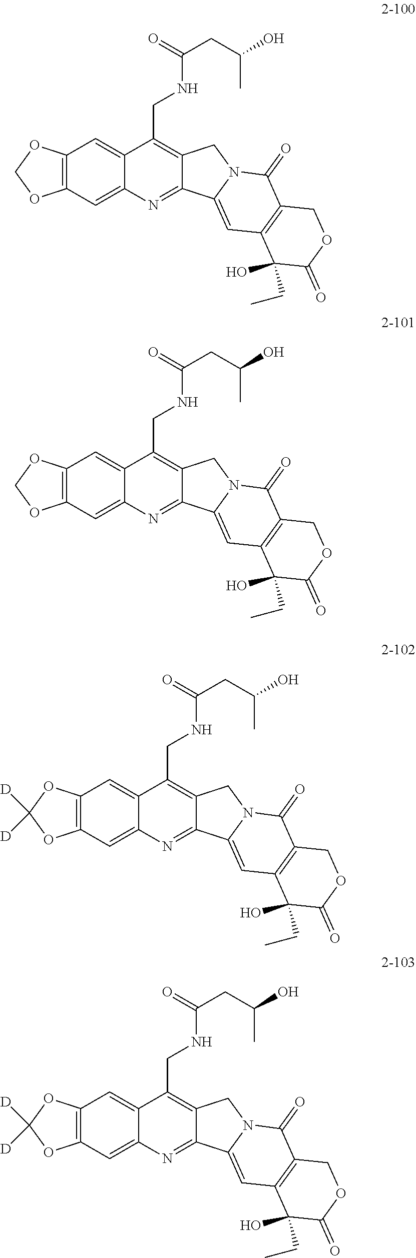 [SMILES: CC[C@@]1(O)C(=O)OCc2c1cc1n(c2=O)Cc2c-1nc1cc3c(cc1c2CNC(=O)C[C@@H](C)O)OCO3 ; CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC]

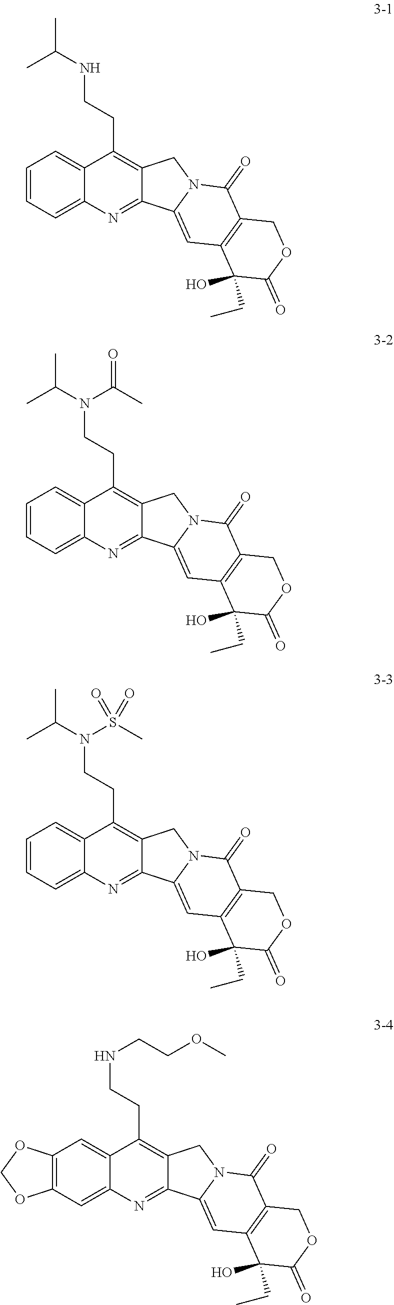 [SMILES: CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3]

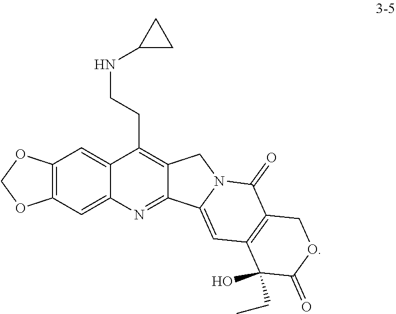 [SMILES: CC[C@@]1(O)C(=O)OCc2c1cc1n(c2=O)Cc2c-1nc1cc3c(cc1c2CCNC1CC1)OCO3]

In some embodiments, the cytotoxic drug is selected from the group consisting of the following compounds or isotopically labeled compounds thereof: 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 2-1, 2-2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 3-1, 3-2, 3-3, 3-4, 3-5.

In some embodiments, the cytotoxic drug is selected from the group consisting of the following compounds or isotopically labeled compounds thereof:

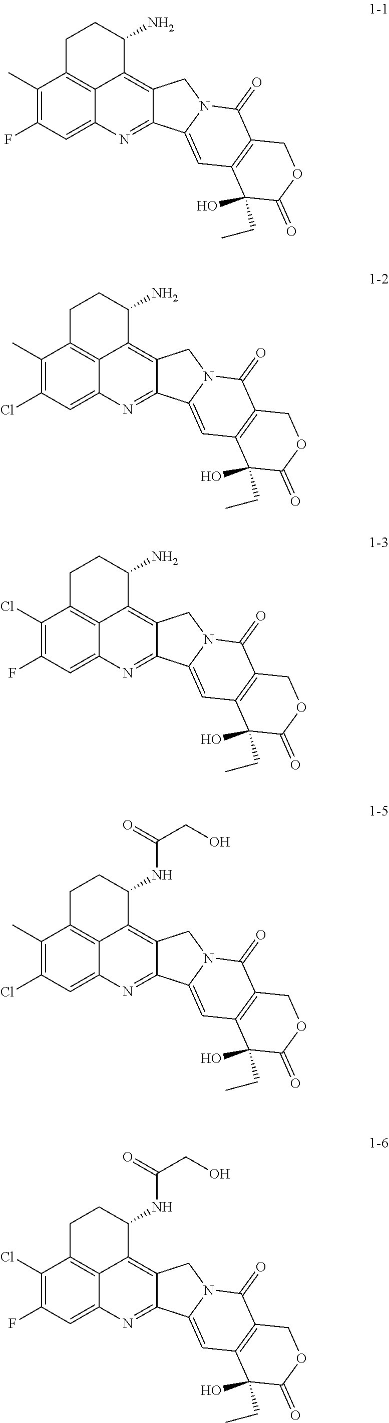 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3]

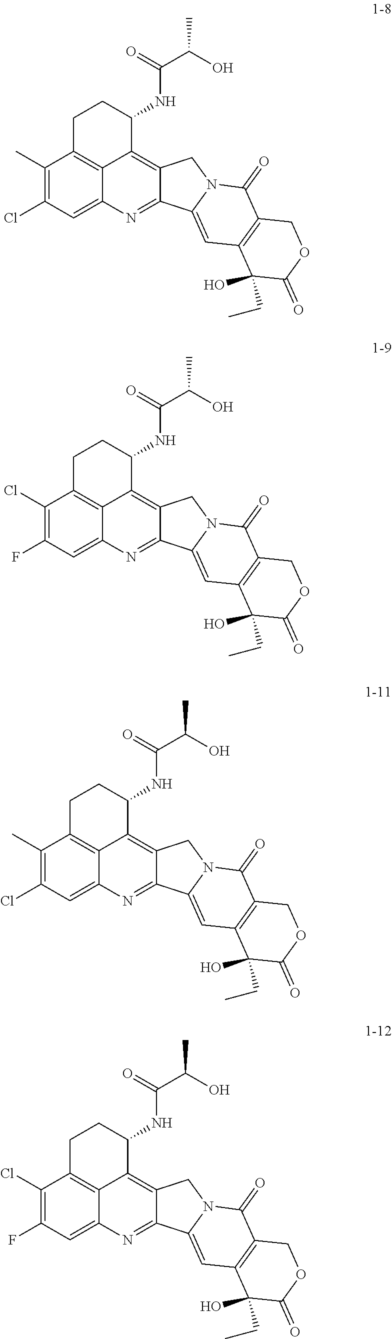 [SMILES: CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@@H](C)O)CC3]

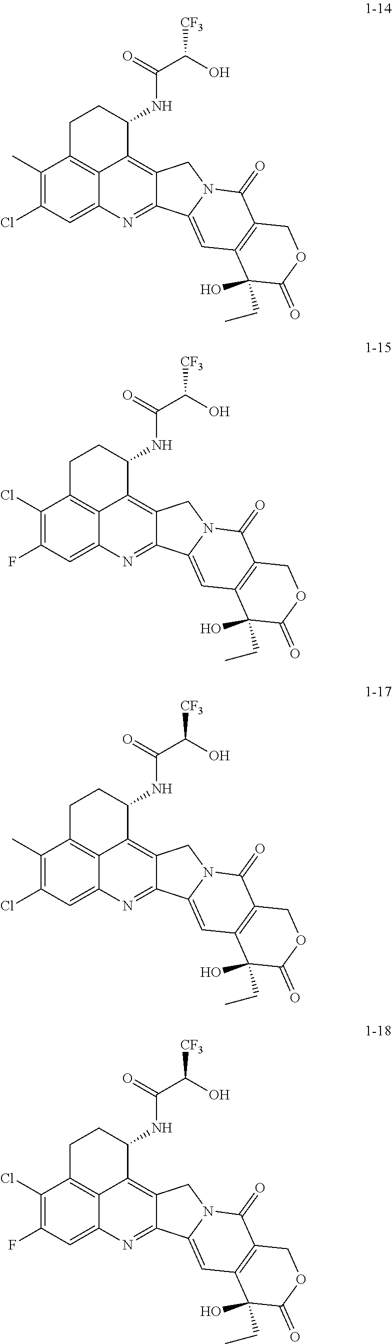 [SMILES: CC[C@@]1(O)C(=O)OCc2c1cc1n(c2=O)Cc2c-1nc1cc(Cl)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3 ; CC[C@@]1(O)C(=O)OCc2c1cc1n(c2=O)Cc2c-1nc1cc(Cl)c(C)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3]

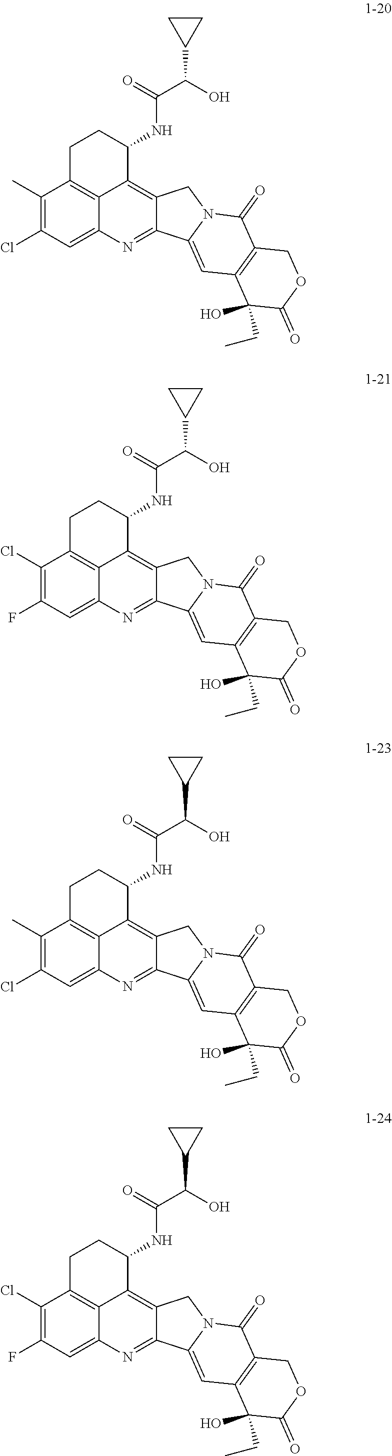 [SMILES: CC[C@@]1(O)C(=O)OCc2c1cc1n(c2=O)Cc2c-1nc1cc(Cl)c(C)c3c1c2[C@@H](NC(=O)[C@@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3]

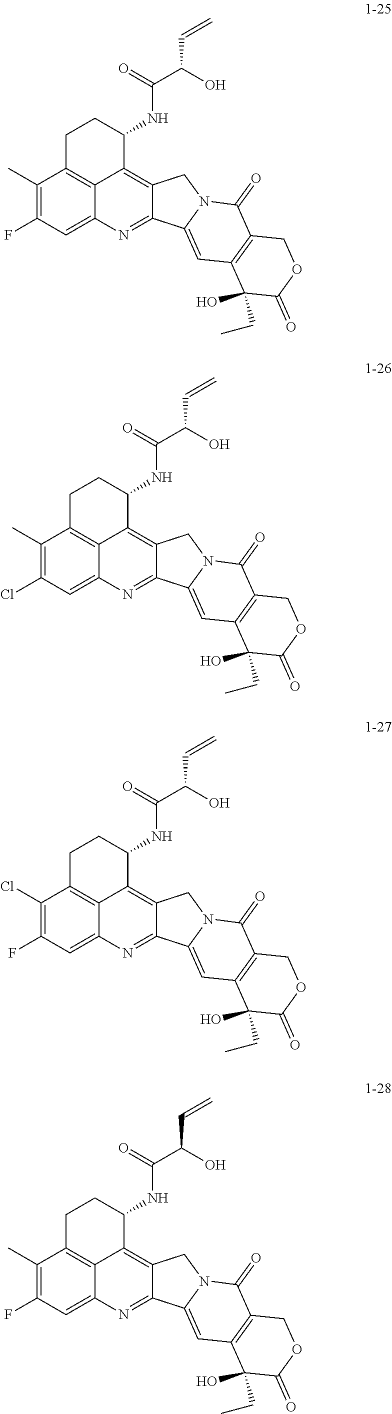

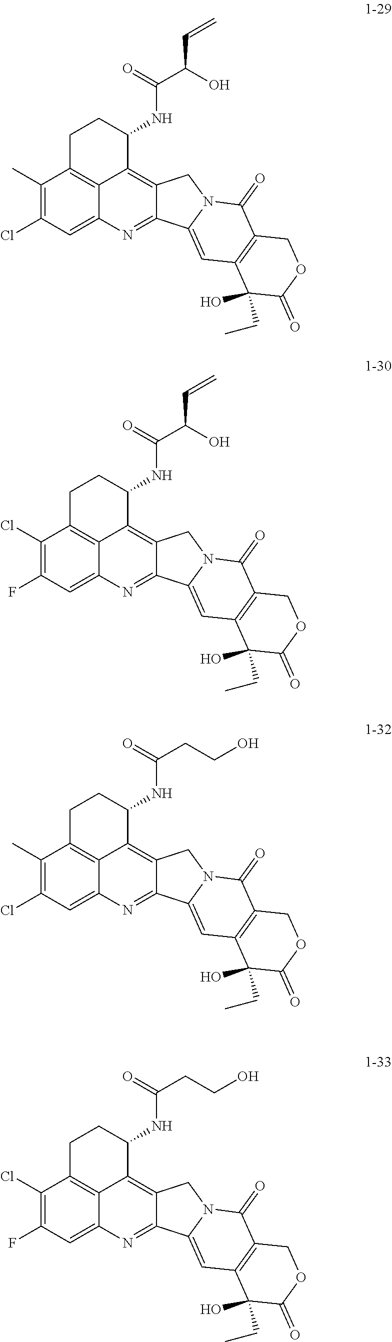

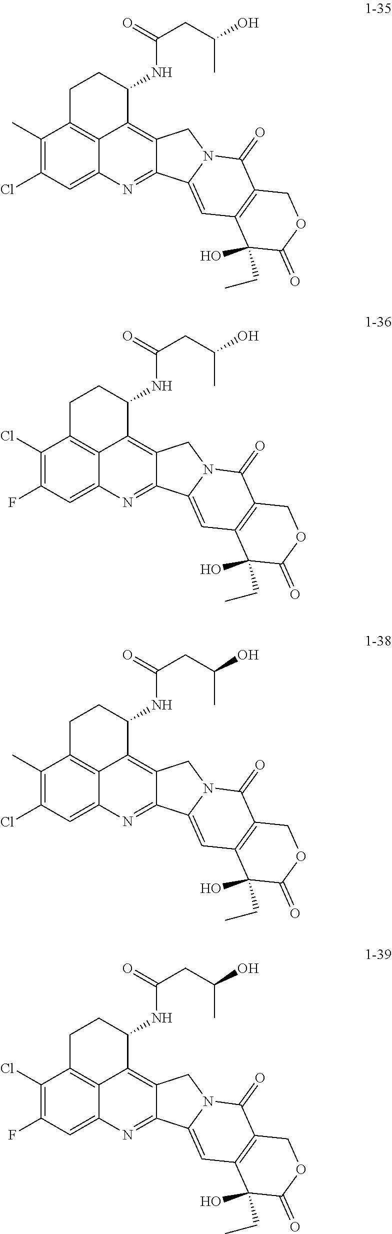 [SMILES: CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(Cl)c(C)c3c1c2[C@@H](NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3]

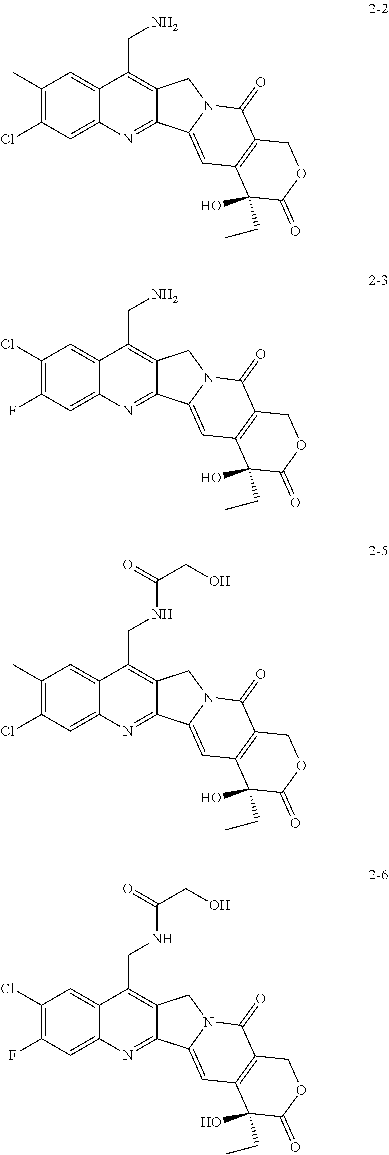 [SMILES: CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO]

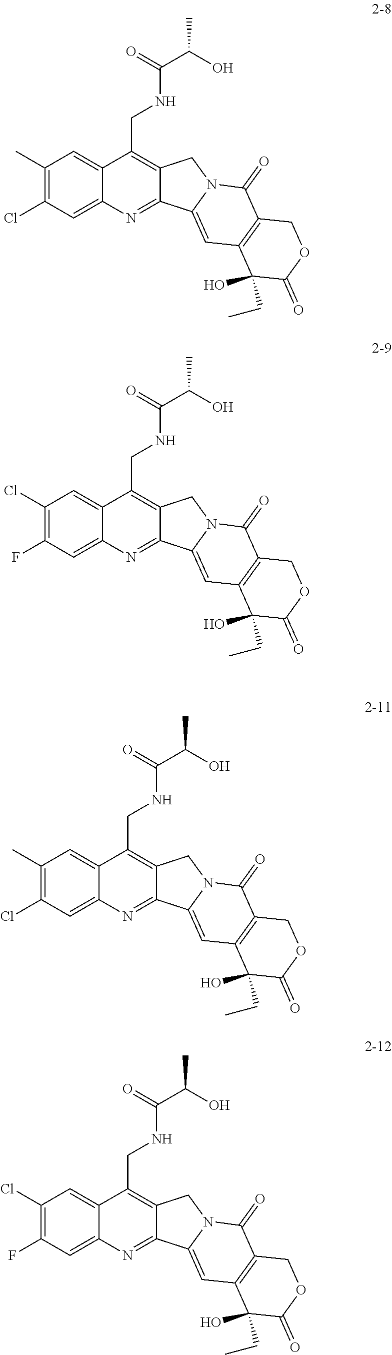 [SMILES: CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O]

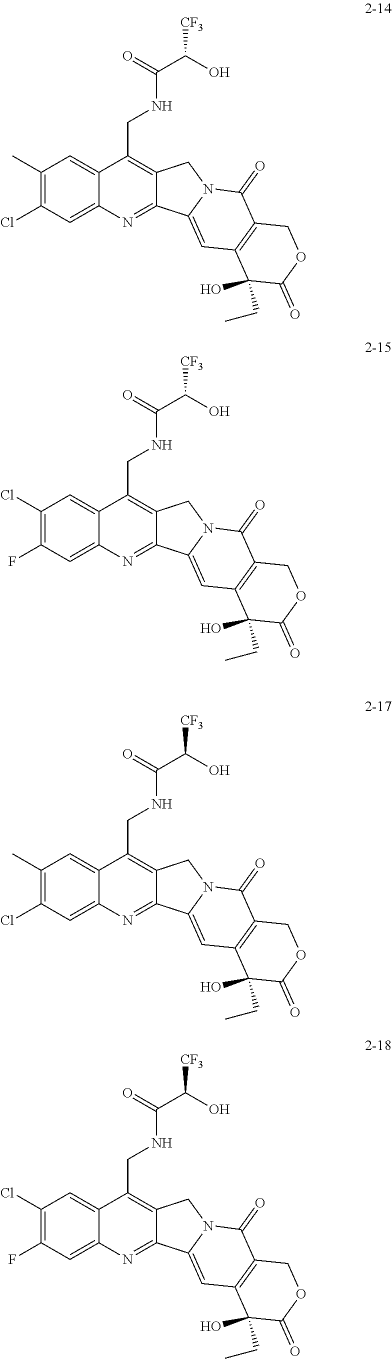 [SMILES: CC[C@@]1(O)C(=O)OCc2c1cc1n(c2=O)Cc2c-1nc1cc(Cl)c(C)cc1c2CNC(=O)[C@@H](O)C(F)(F)F ; CC[C@@]1(O)C(=O)OCc2c1cc1n(c2=O)Cc2c-1nc1cc(F)c(Cl)cc1c2CNC(=O)[C@@H](O)C(F)(F)F ; CC[C@@]1(O)C(=O)OCc2c1cc1n(c2=O)Cc2c-1nc1cc(Cl)c(C)cc1c2CNC(=O)[C@H](O)C(F)(F)F ; CC[C@@]1(O)C(=O)OCc2c1cc1n(c2=O)Cc2c-1nc1cc(F)c(Cl)cc1c2CNC(=O)[C@H](O)C(F)(F)F]

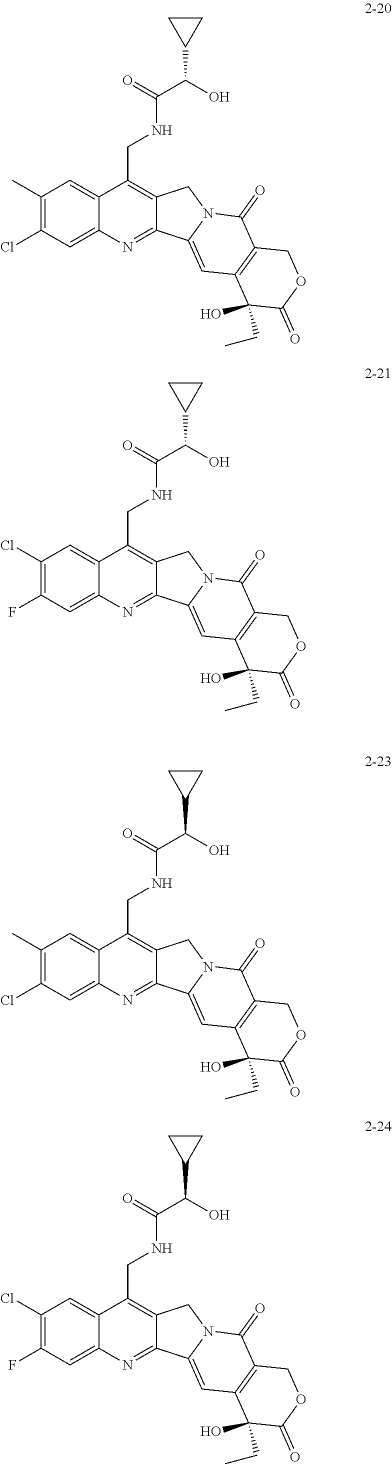

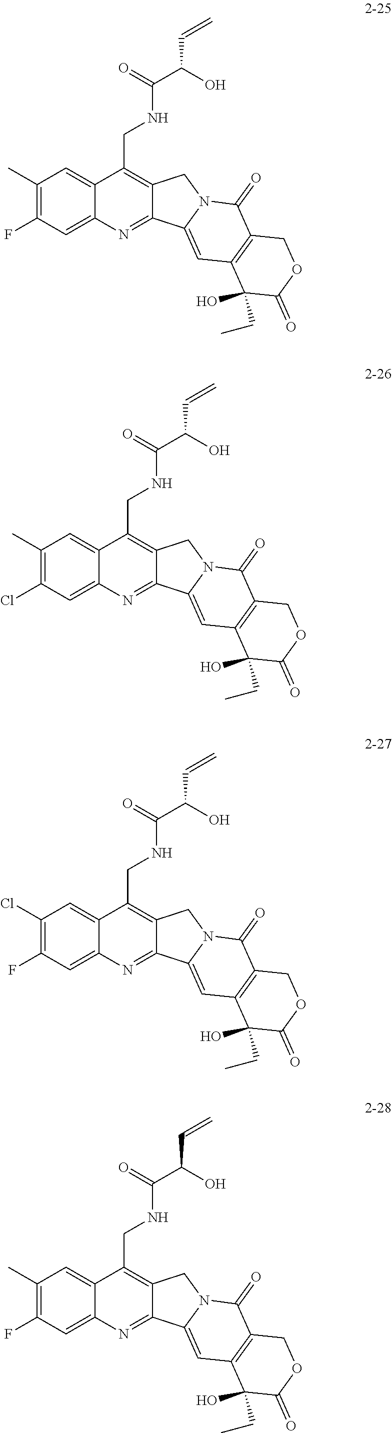 [SMILES: C=C[C@H](O)C(=O)NCc1c2c(nc3cc(F)c(C)cc13)-c1cc3c(c(=O)n1C2)COC(=O)[C@]3(O)CC ; C=C[C@H](O)C(=O)NCc1c2c(nc3cc(Cl)c(C)cc13)-c1cc3c(c(=O)n1C2)COC(=O)[C@]3(O)CC ; C=C[C@H](O)C(=O)NCc1c2c(nc3cc(F)c(Cl)cc13)-c1cc3c(c(=O)n1C2)COC(=O)[C@]3(O)CC ; C=C[C@@H](O)C(=O)NCc1c2c(nc3cc(F)c(C)cc13)-c1cc3c(c(=O)n1C2)COC(=O)[C@]3(O)CC]

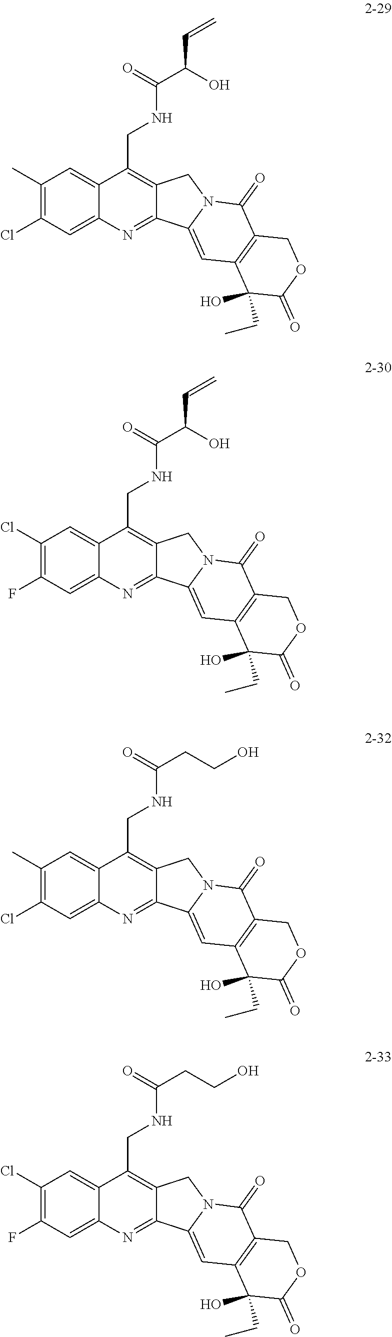

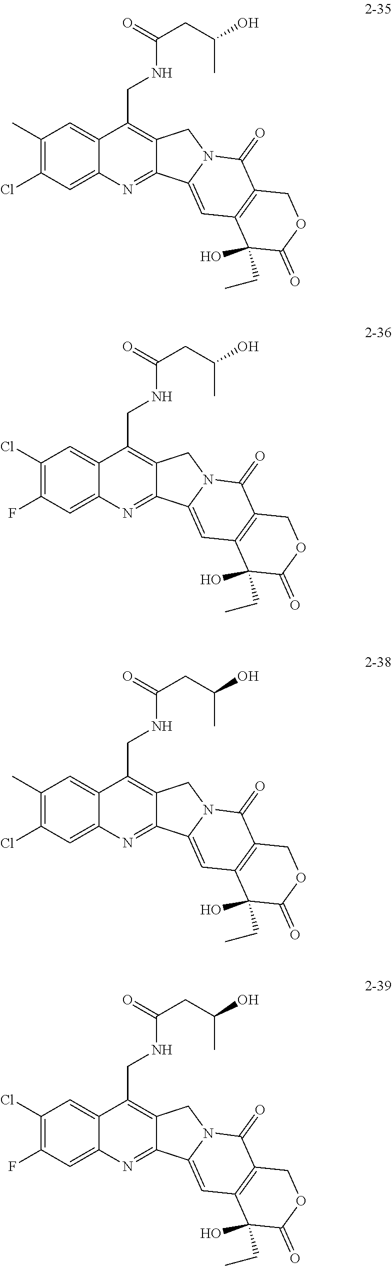 [SMILES: CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O]

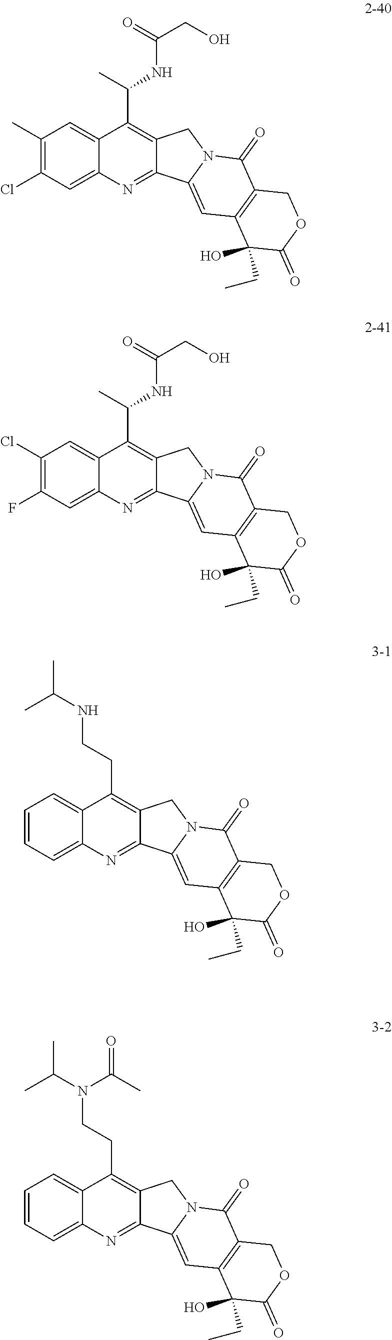 [SMILES: CC[C@@]1(O)C(=O)OCc2c1cc1n(c2=O)Cc2c-1nc1cc(Cl)c(C)cc1c2[C@H](C)NC(=O)CO ; CC[C@@]1(O)C(=O)OCc2c1cc1n(c2=O)Cc2c-1nc1cc(F)c(Cl)cc1c2[C@H](C)NC(=O)CO ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C]

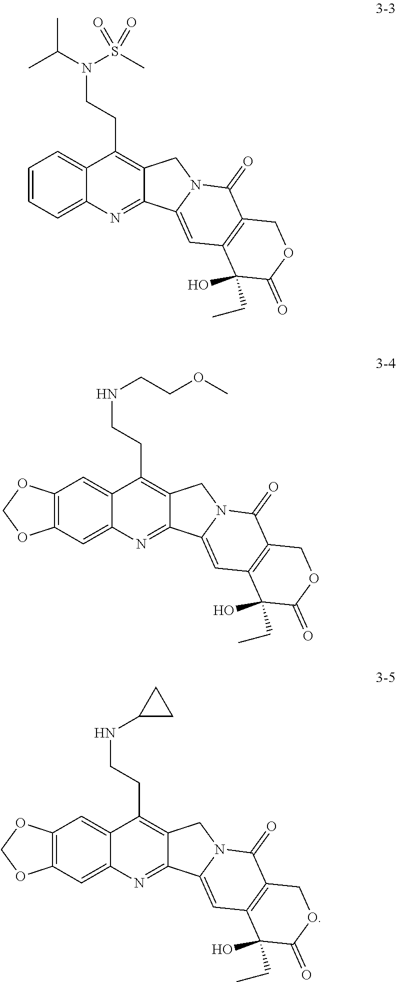 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1cc3c(cc1c2CCNC1CC1)OCO3]

Those skilled in the art should understand that the antibody-drug conjugate described in the present application can be prepared modularly. For example, a compound of a free form of a “drug-linker” (which can be understood as M′-L-E-D, wherein M′ represents the structural form of M before covalently connected to the antibody or antigen-binding fragment thereof) is first obtained, and then it is covalently connected to the antibody or antigen-binding fragment thereof to obtain the antibody-drug conjugate as described in the present application. Accordingly, M′ in the free form of the “drug-linker” is connected to one or more sulfhydryl groups (—SH) or amino groups (—NH₂) on the antibody or antigen-binding fragment thereof by a substitution reaction (e.g., removal of a structure such as —SO₂Me or pentafluorophenol thereon) or by an addition reaction.

In one aspect, the present application provides a compound or pharmaceutically acceptable salt thereof, which has a structure represented by formula D-E-L-M′, wherein:
M′ represents -M-Lg, wherein Lg is a leaving group for a nucleophilic substitution reaction, and M is a structural fragment capable of binding to an antibody or antigen-binding fragment thereof;
L represents a structural fragment connecting M and E;
E represents a structural fragment connecting L and D;
D represents a cytotoxic drug fragment.

In some embodiments, Lg is selected from the group consisting of halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfonate group, C₁₋₆ alkylsulfoxide group, halogenated phenoxy, hydroxyl (—OH), sulfhydryl (—SH), amino (—NH₂), nitro, azido, cyano, alkenyl, alkynyl, and alkynyl-containing structural fragment, wherein the halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfonate group, C₁₋₆ alkylsulfoxide group, halogenated phenoxy, alkenyl, alkynyl, and alkynyl-containing structural fragment are optionally substituted with one or more suitable substituents.

In some embodiments, Lg is selected from the group consisting of halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfonate group, C₁₋₆ alkylsulfoxide group, halogenated phenoxy, hydroxyl (—OH), sulfhydryl (—SH), amino (—NH₂), nitro, azido, cyano, alkenyl, alkynyl, and alkynyl-containing structural fragment.

In some embodiments, Lg is selected from the group consisting of halogen, substituted or unsubstituted C₁₋₆ alkylsulfonyl (C₁₋₆ alkyl-SO₂—), halogenated phenoxy, hydroxyl (—OH), sulfhydryl (—SH), or amino (—NH₂).

In some embodiments, Lg is selected from the group consisting of halogen, substituted or unsubstituted methylsulfonyl, halogenated phenoxy, hydroxyl (—OH), sulfhydryl (—SH), or amino (—NH₂).

In some embodiments, Lg is selected from the group consisting of C₁₋₆ alkylsulfonyl and halogenated phenoxy.

In some embodiments, Lg is selected from the group consisting of methylsulfonyl or pentafluorophenoxy.

In some embodiments, the compound or pharmaceutically acceptable salt thereof is selected from the group consisting of E-1 to E-50, F-1 to F-14, G-1 to G-26, I-1 to I-50 as shown below:

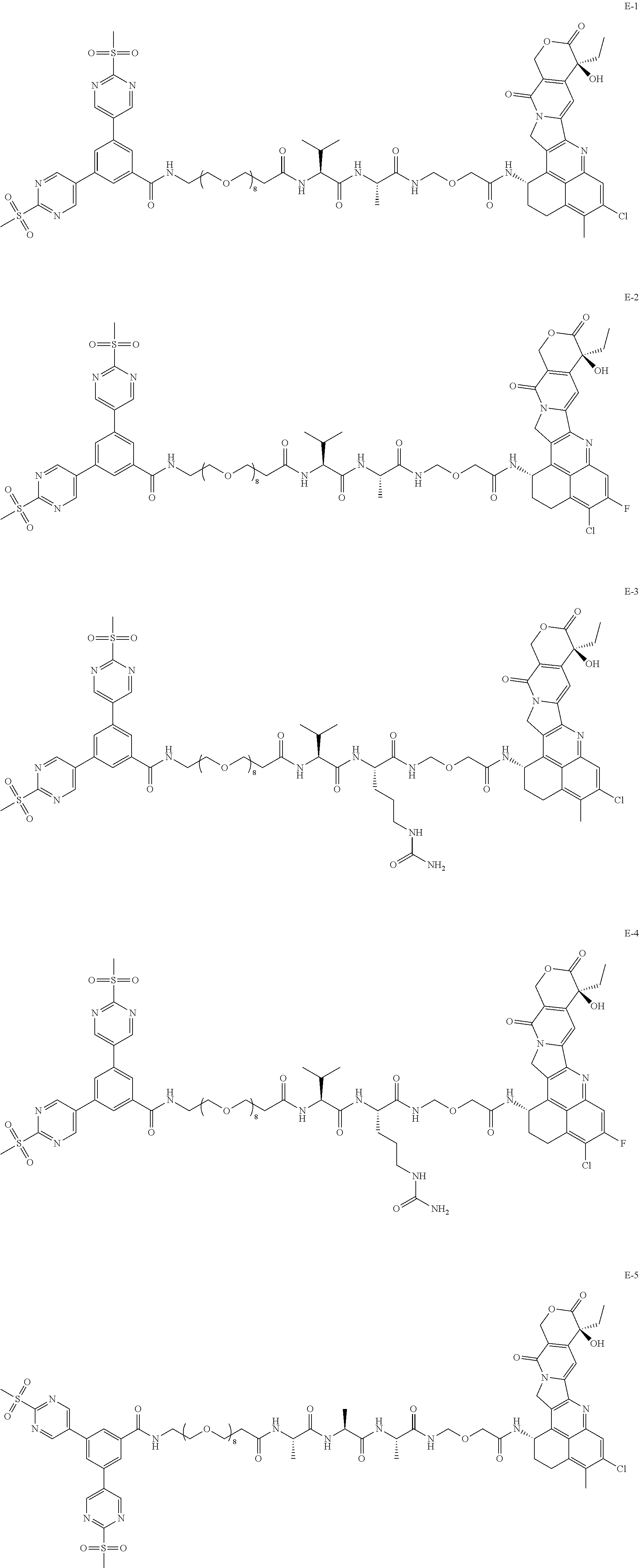

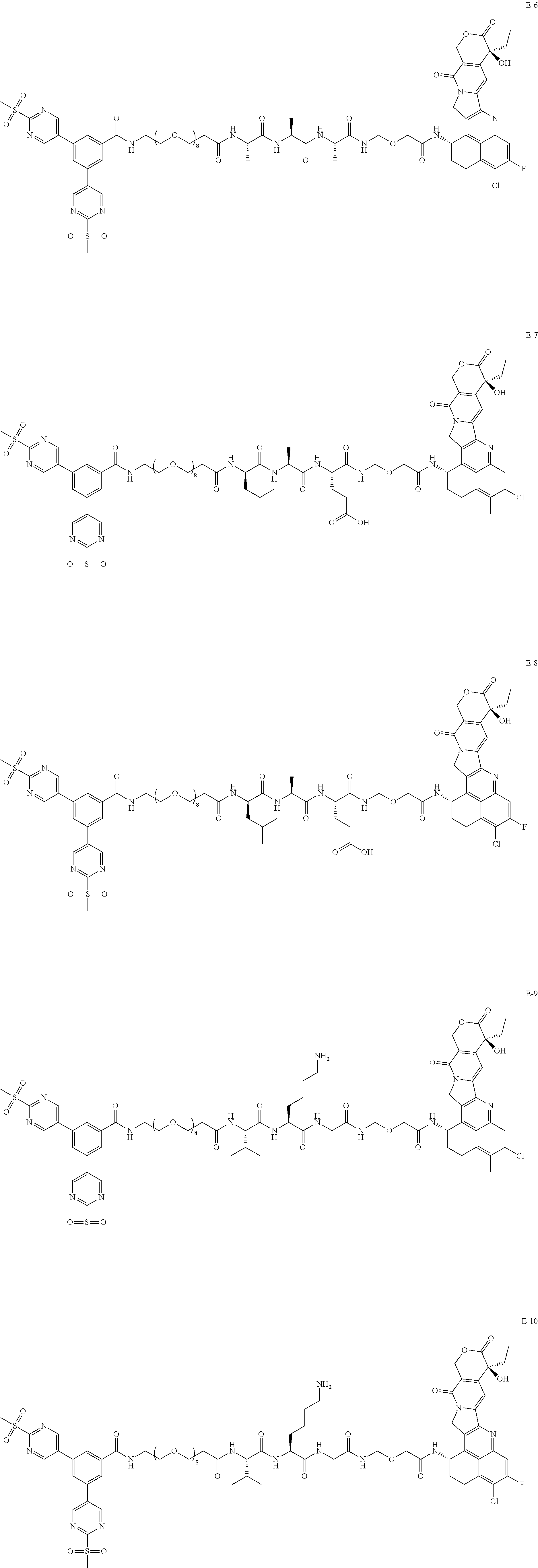

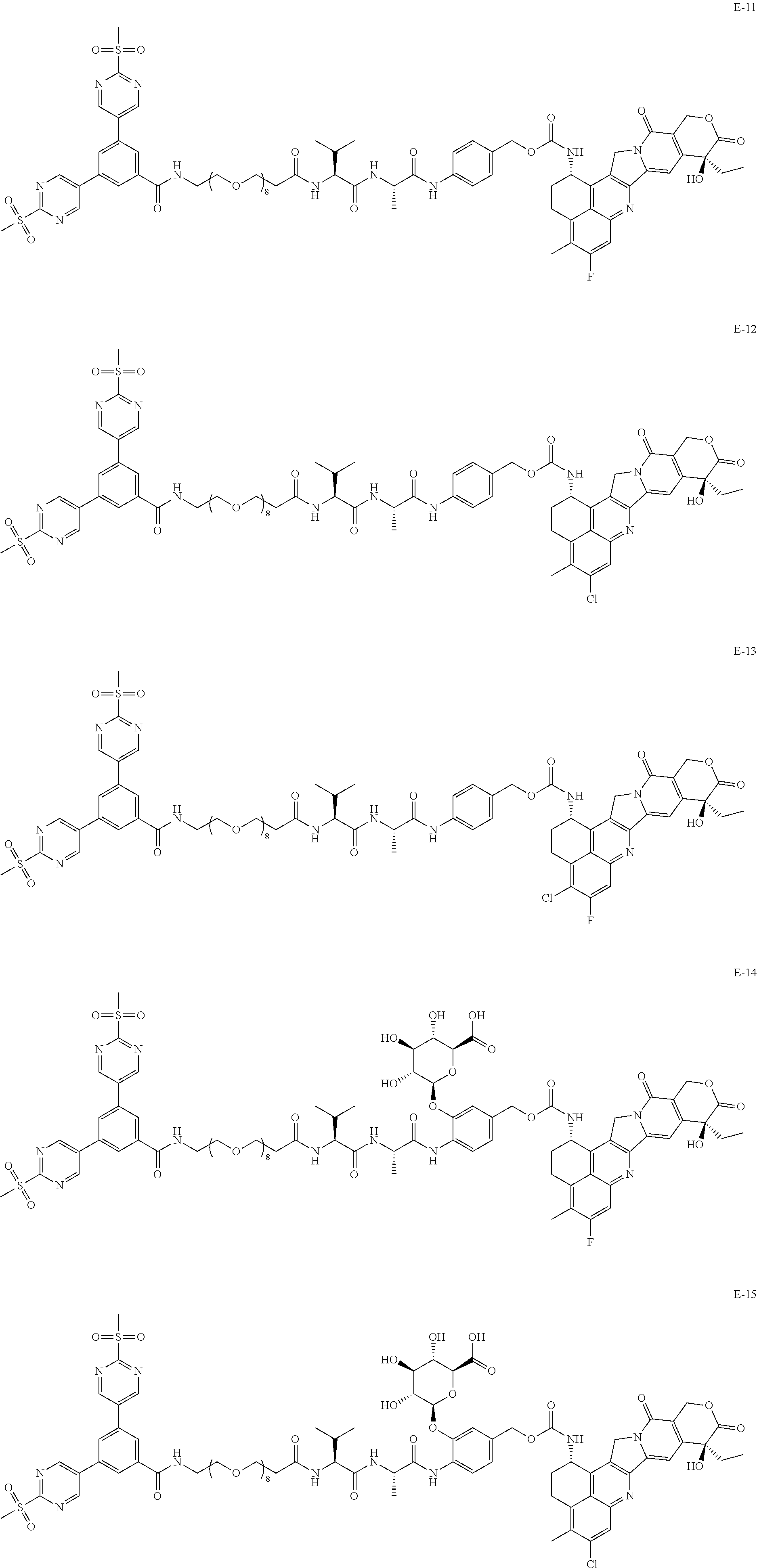

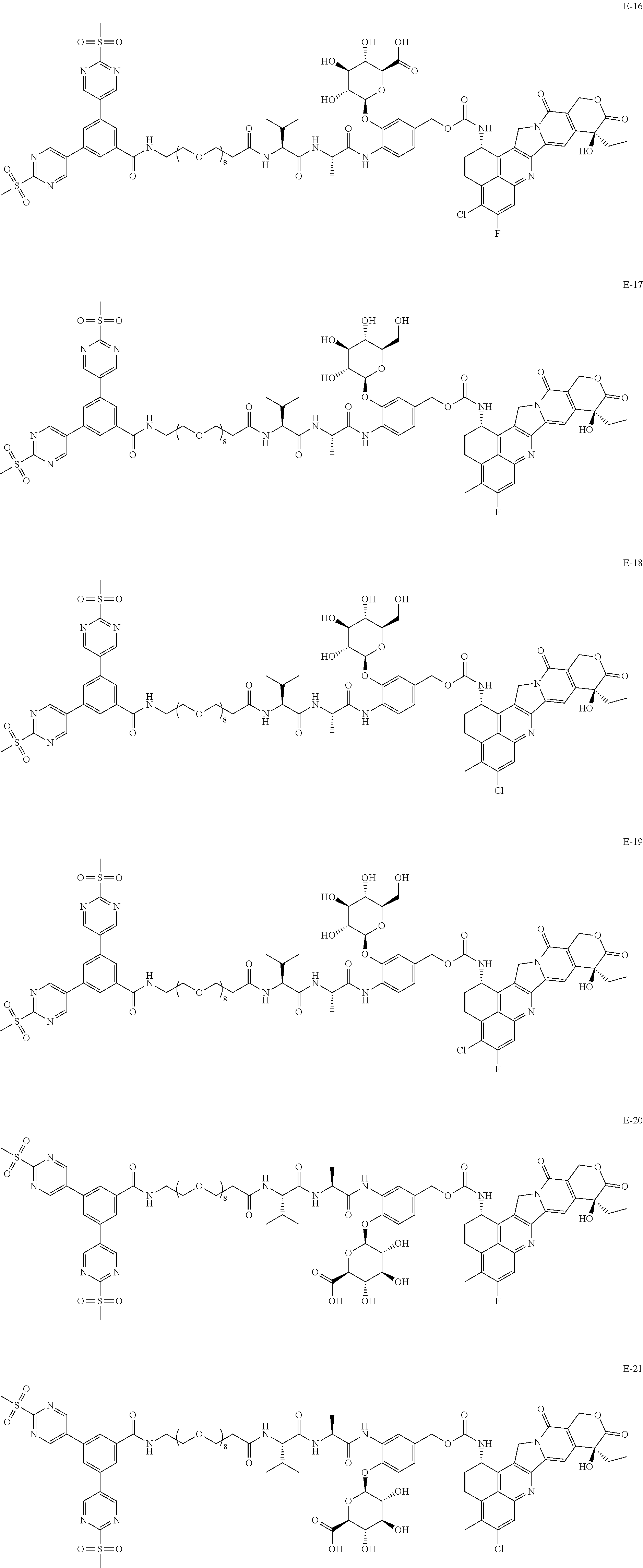

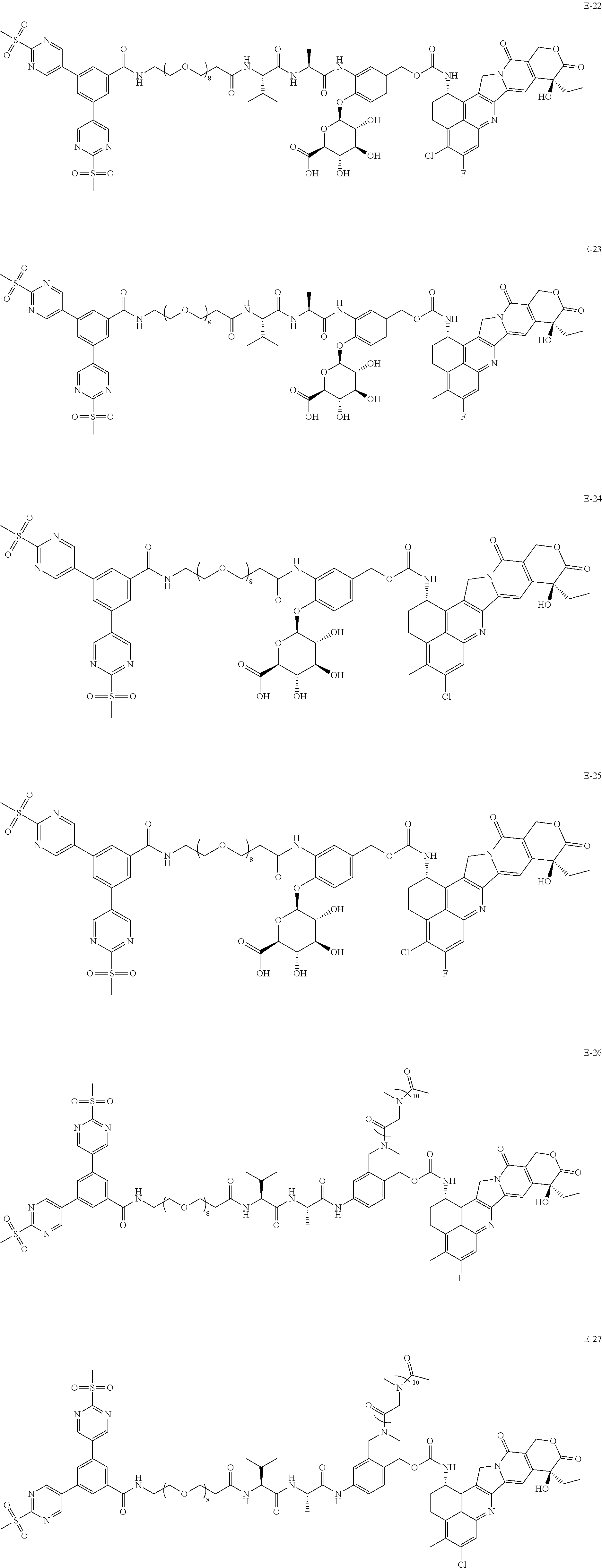

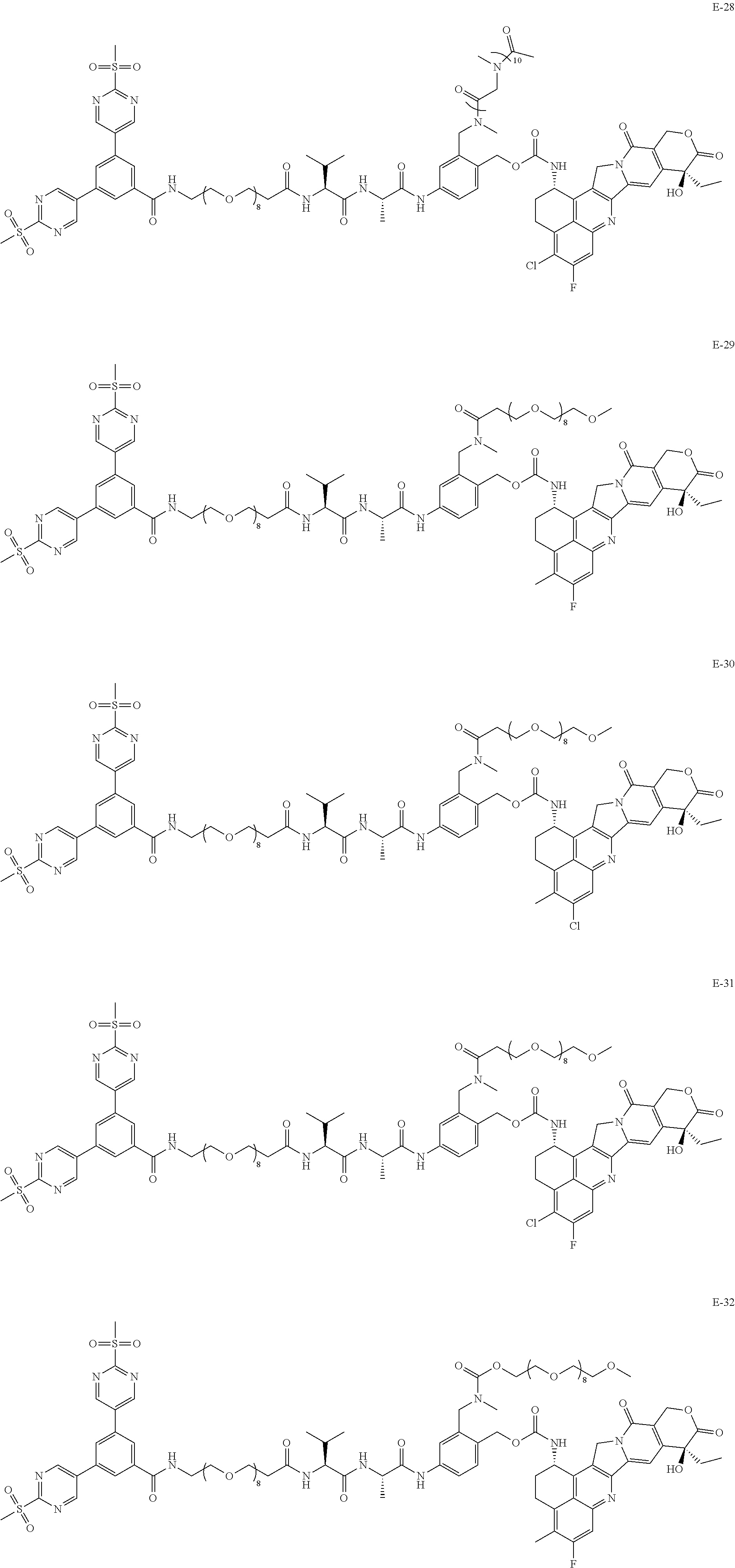

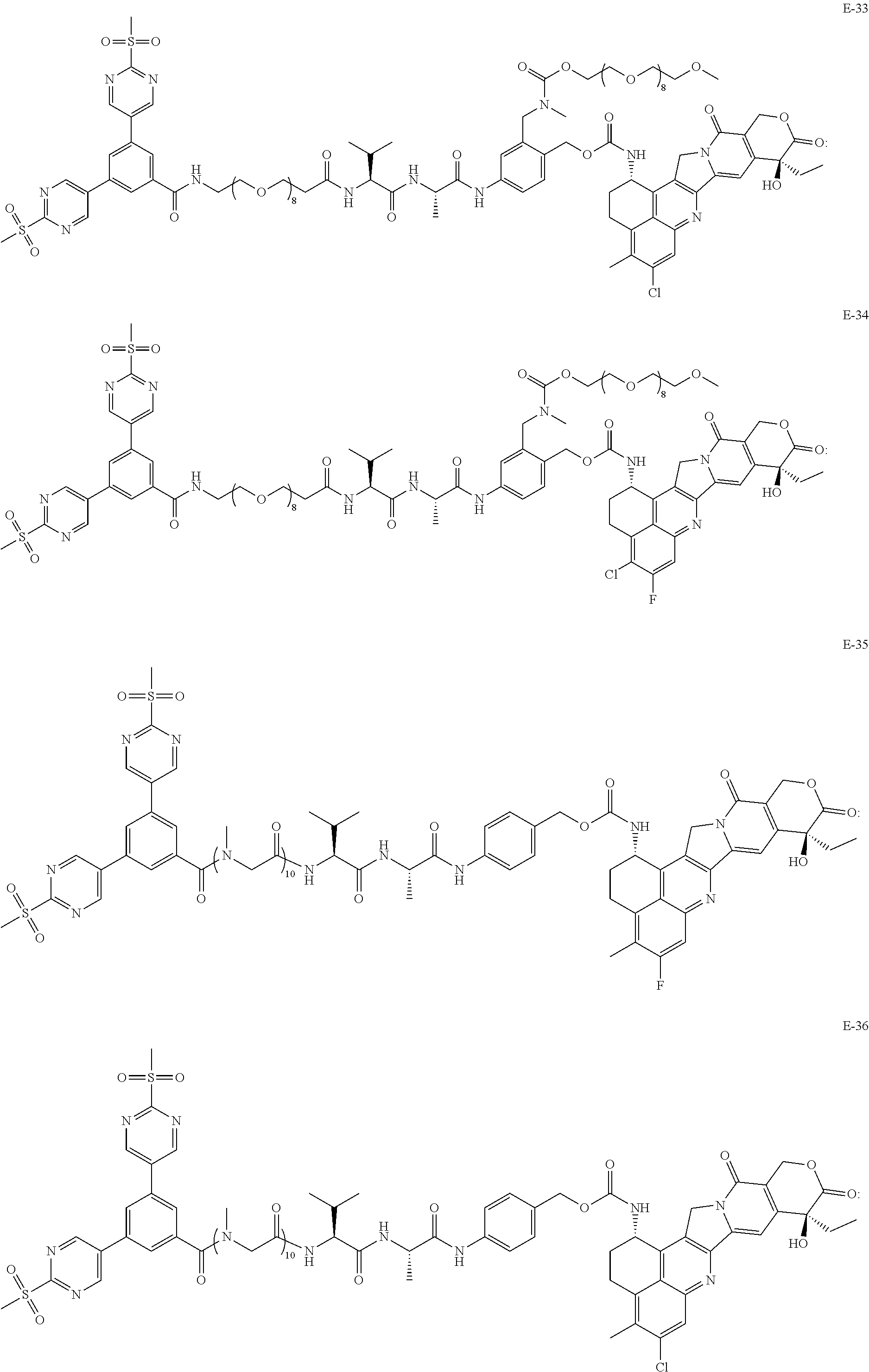

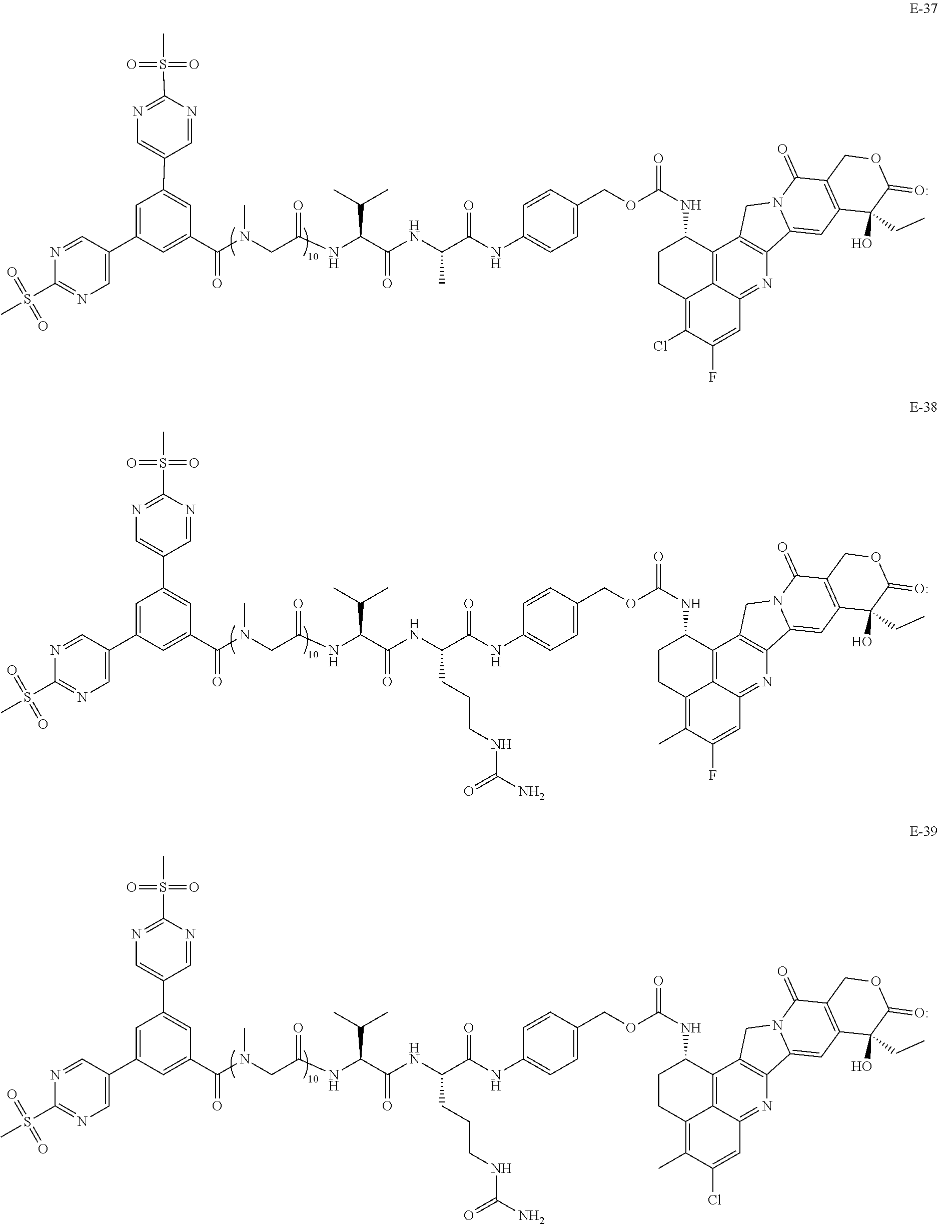

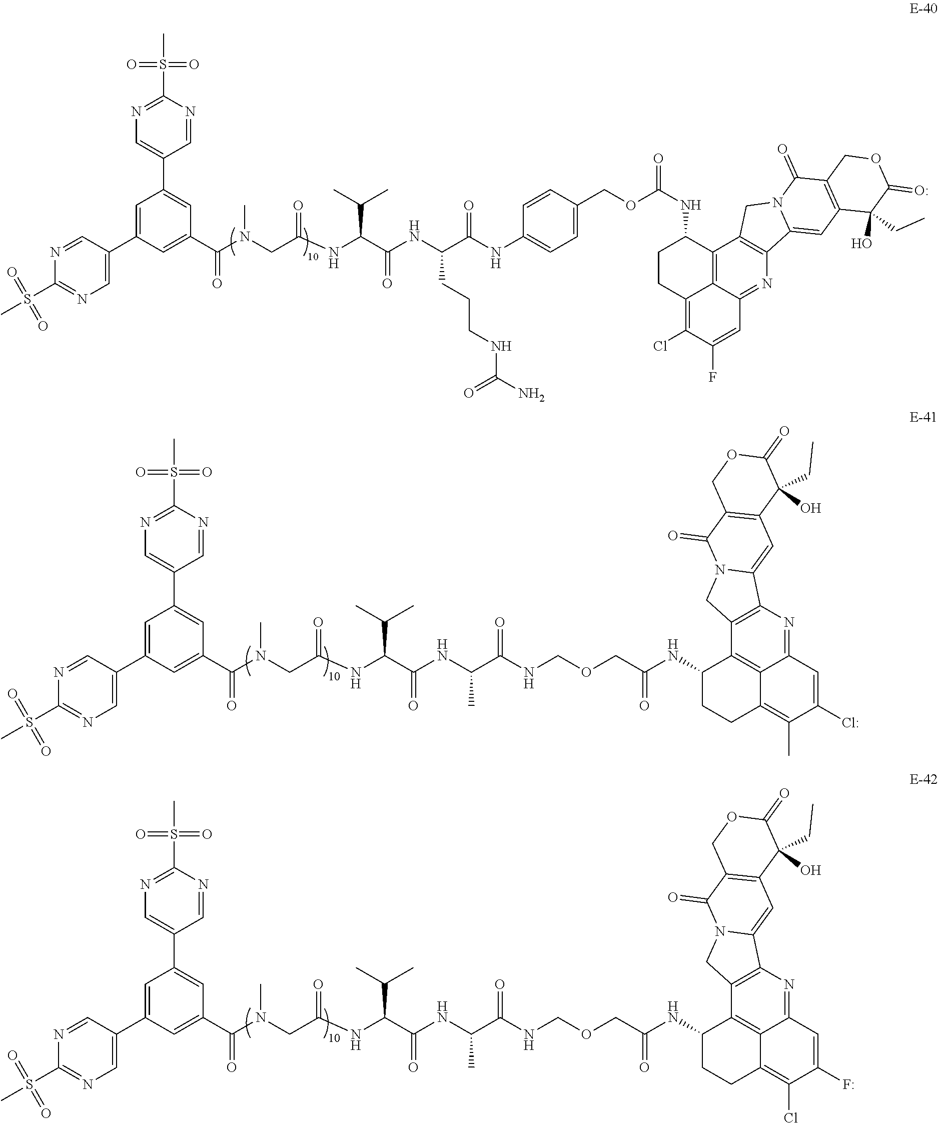

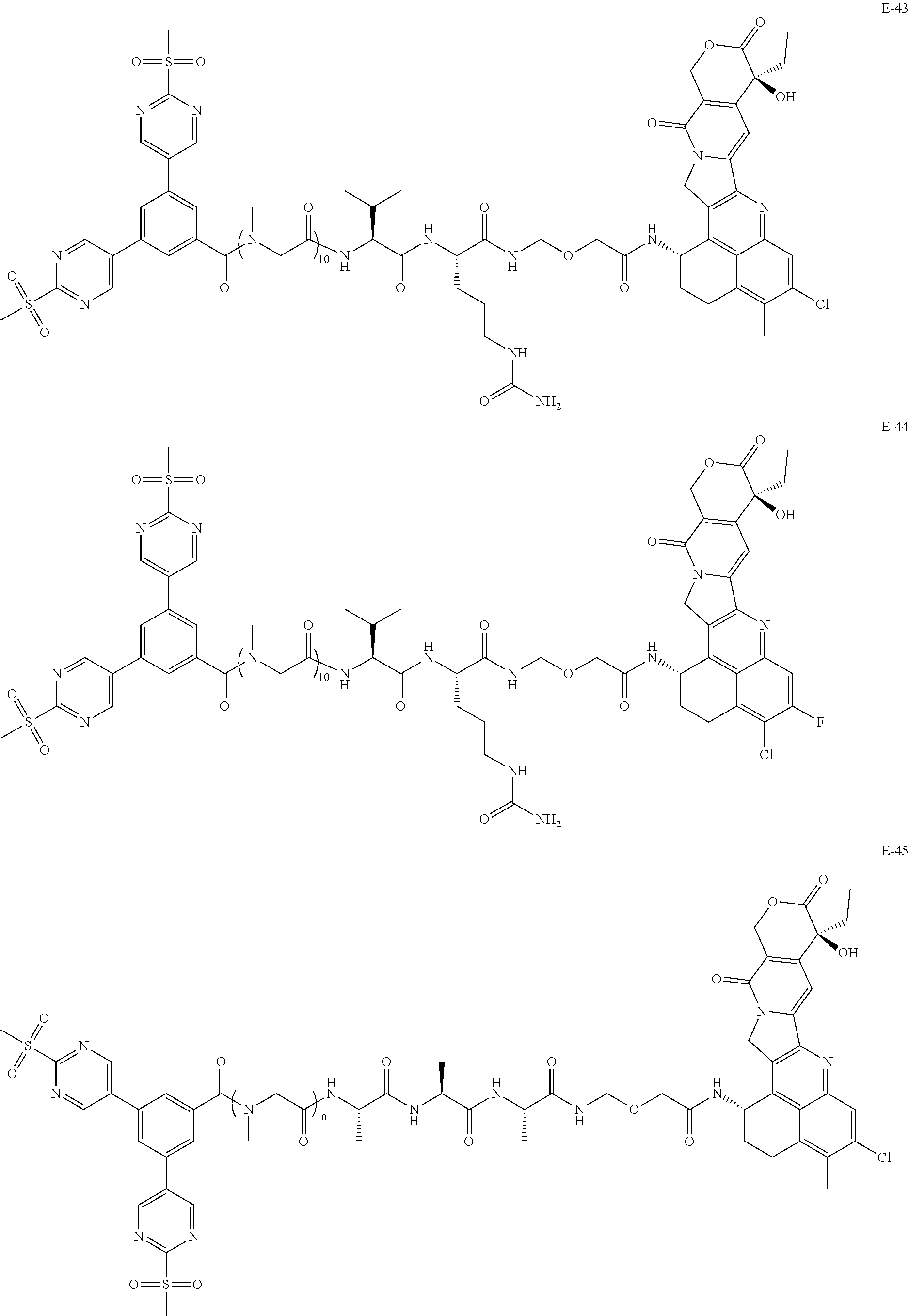

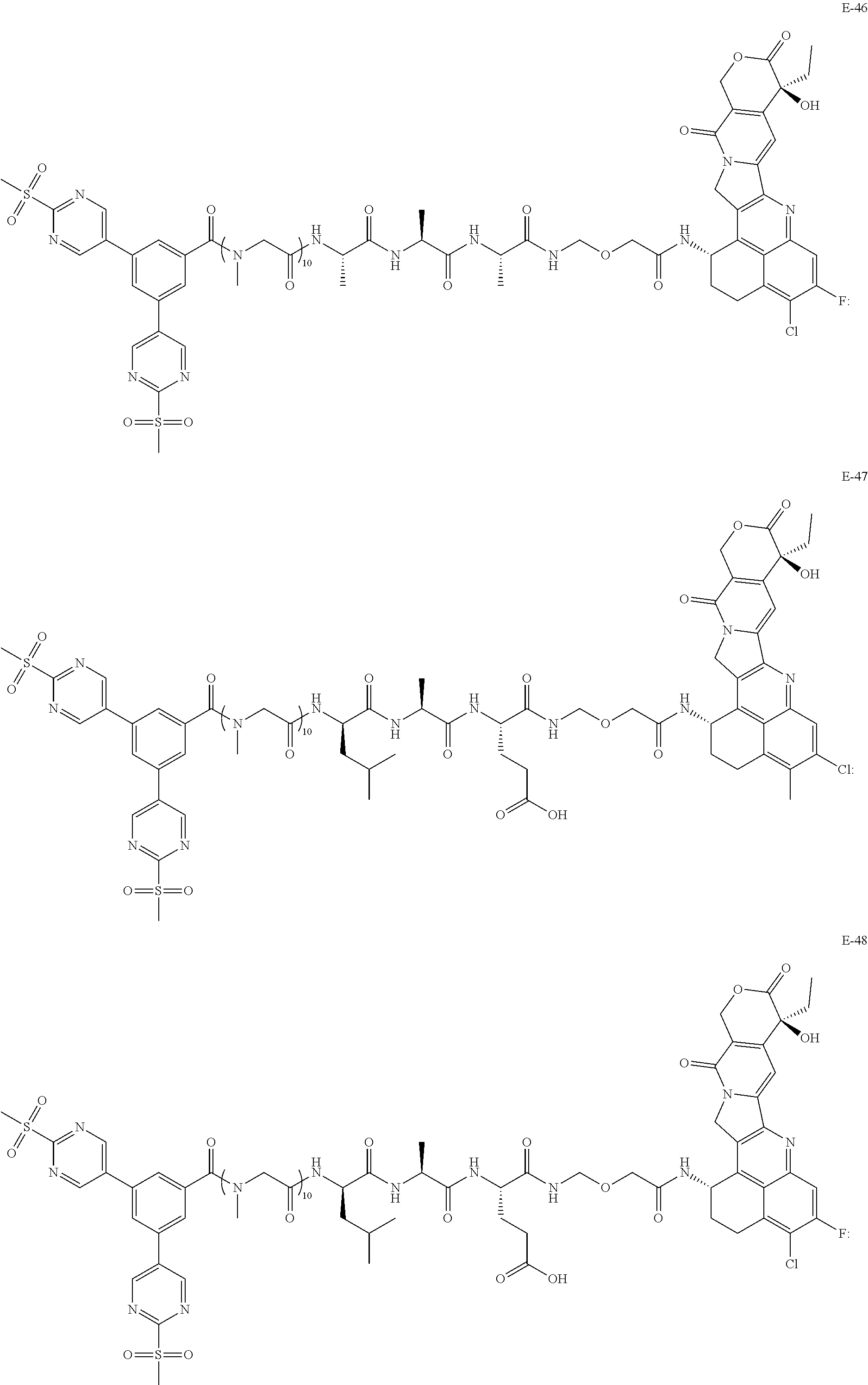

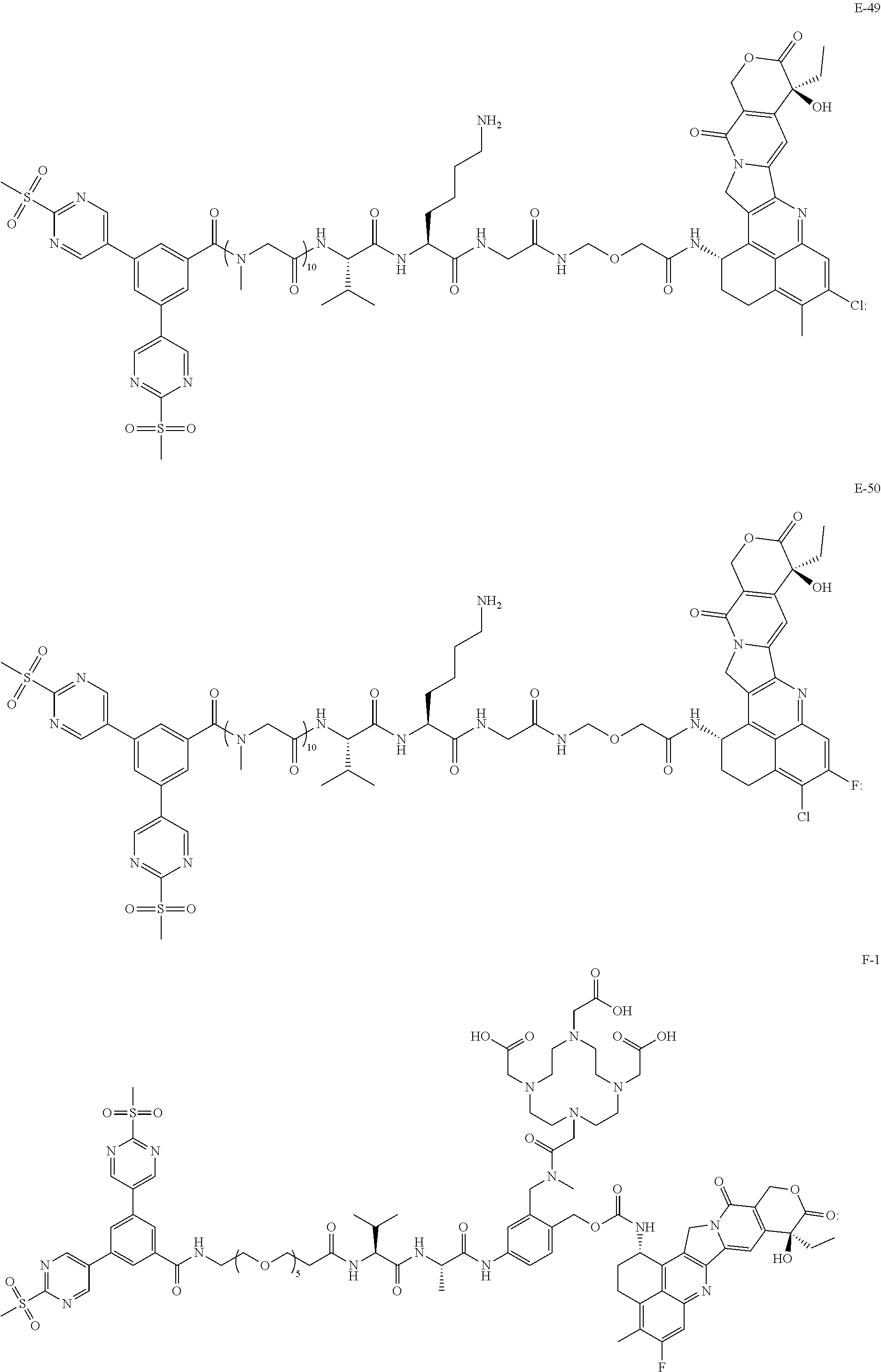

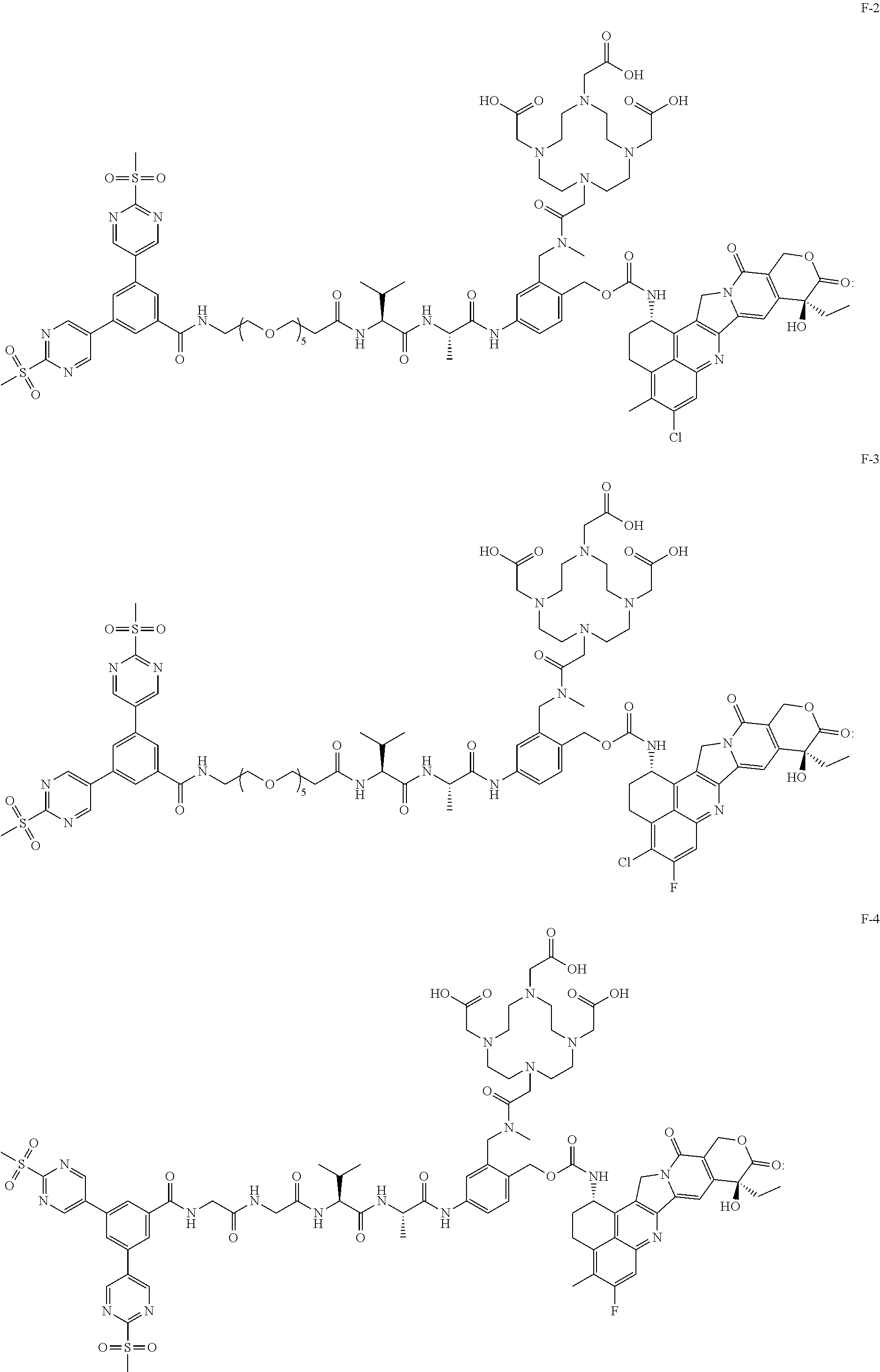

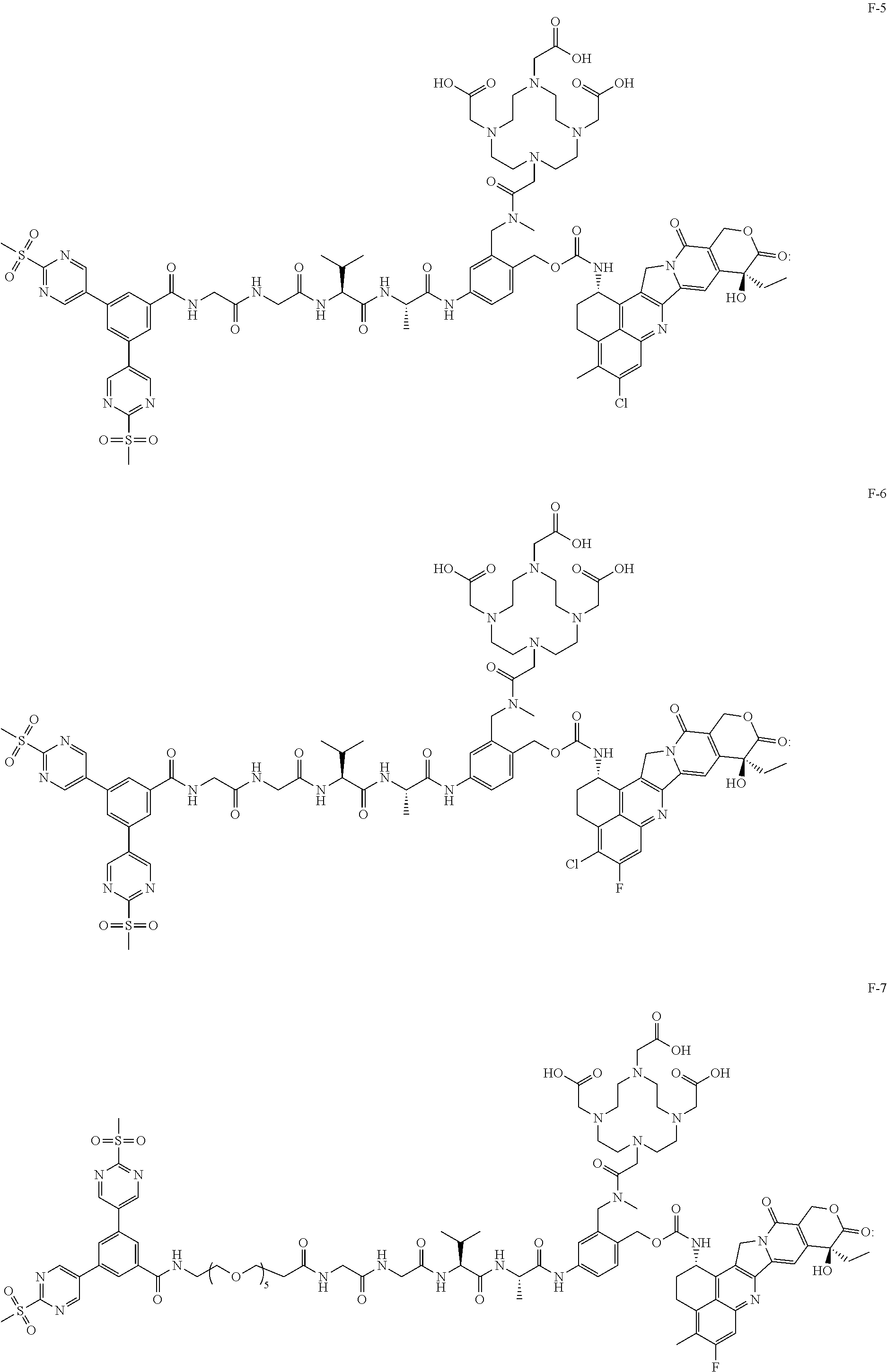

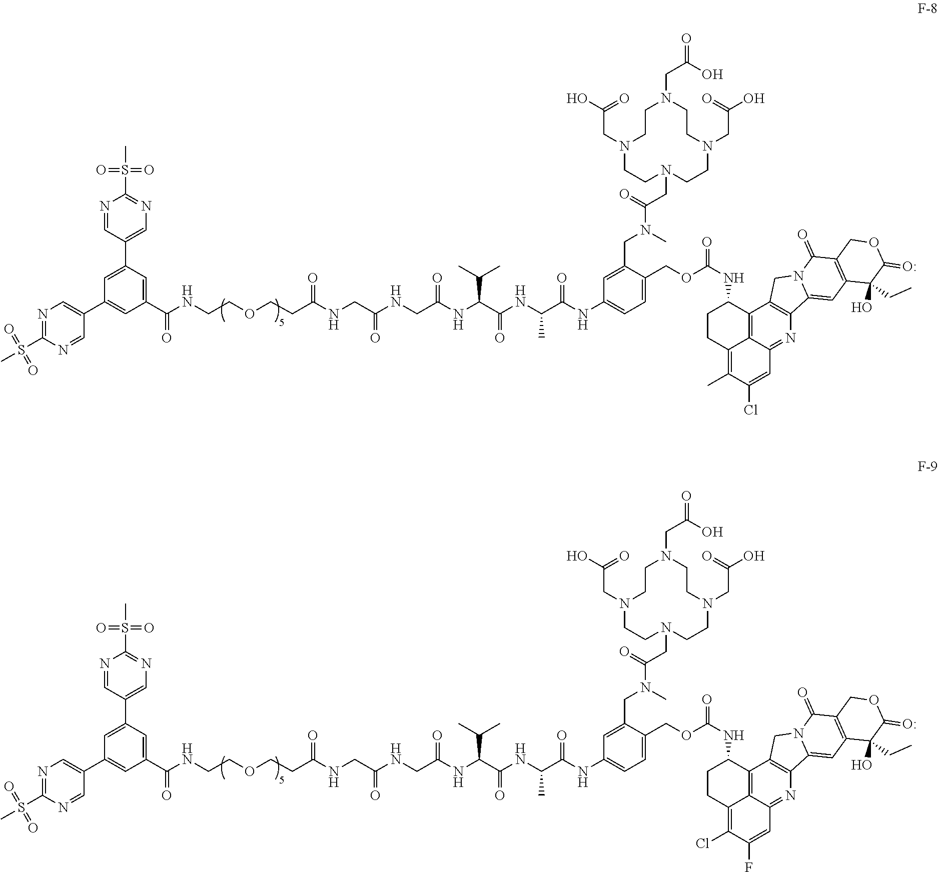

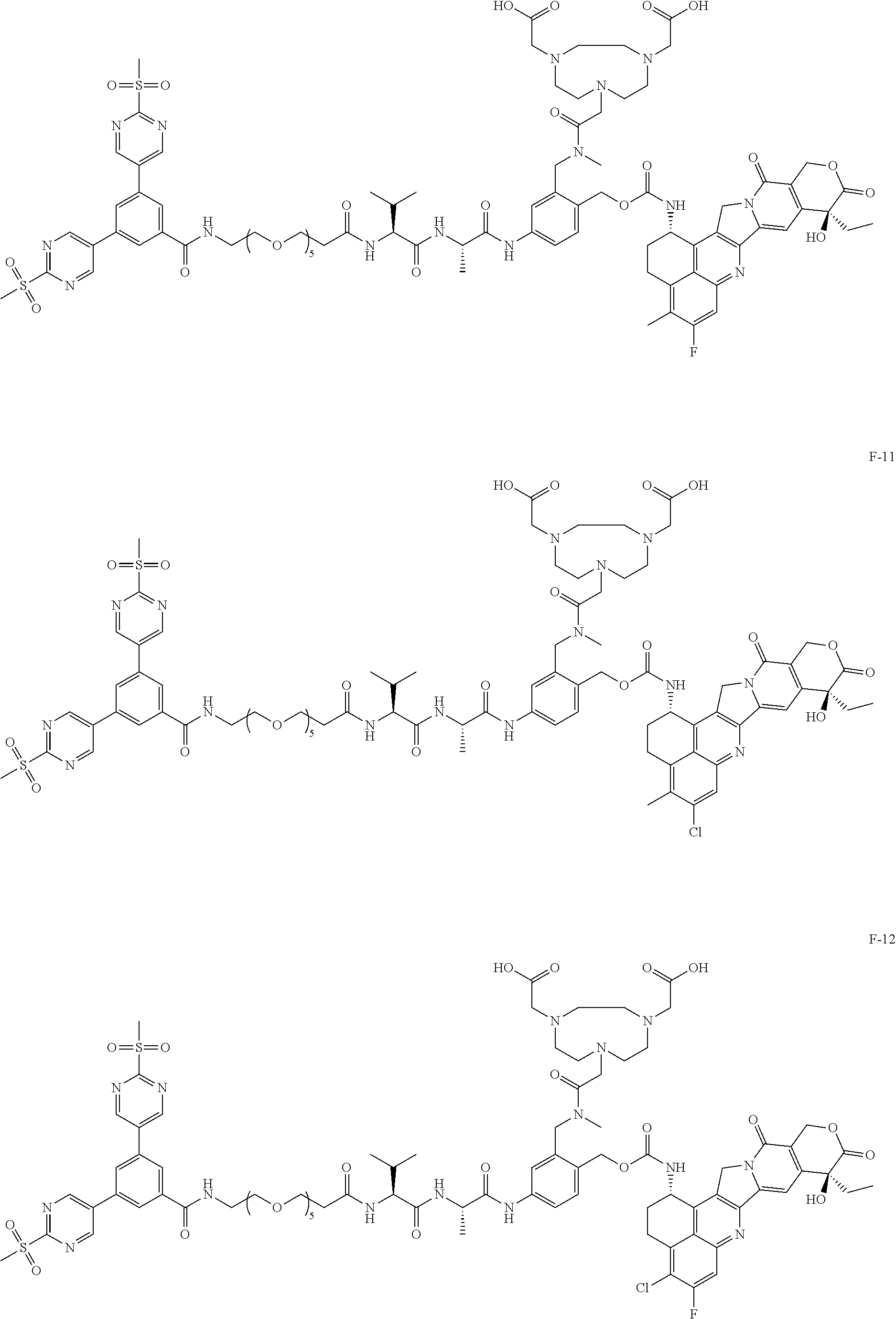

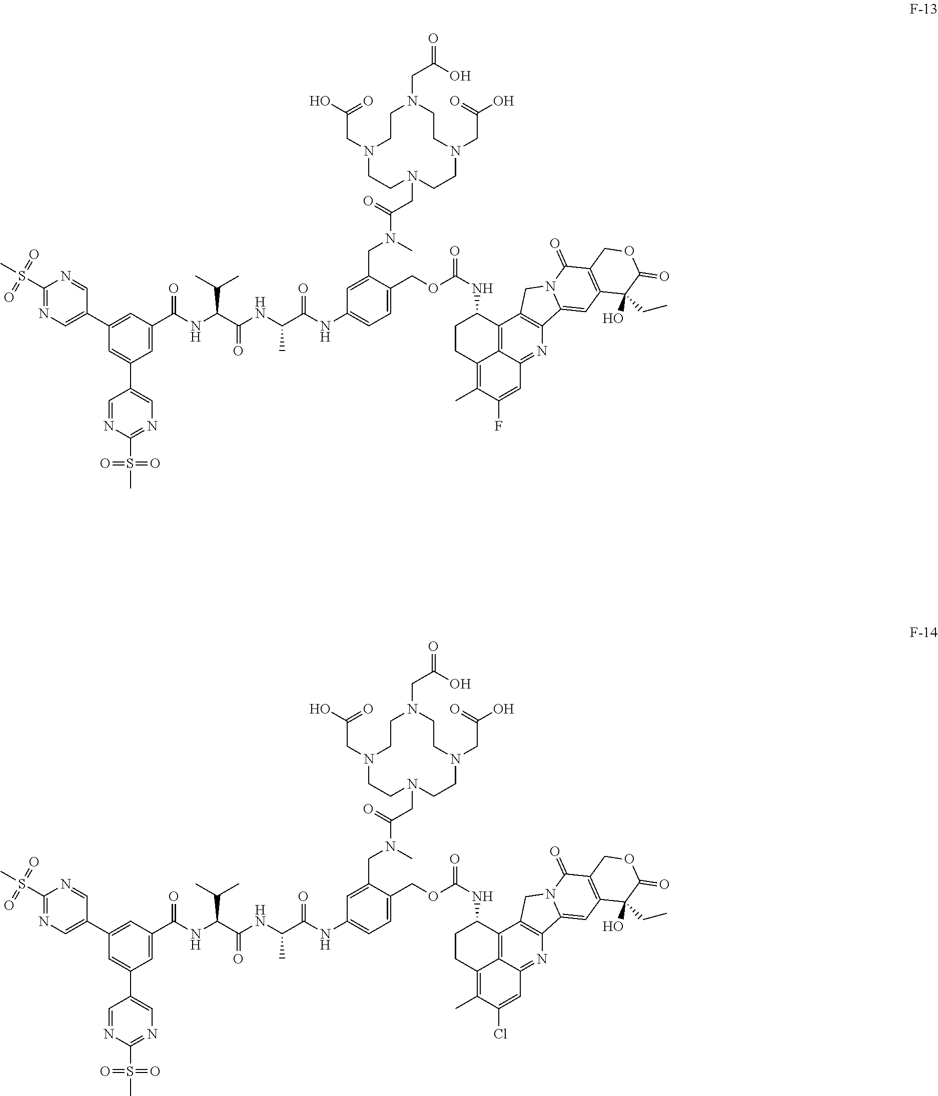

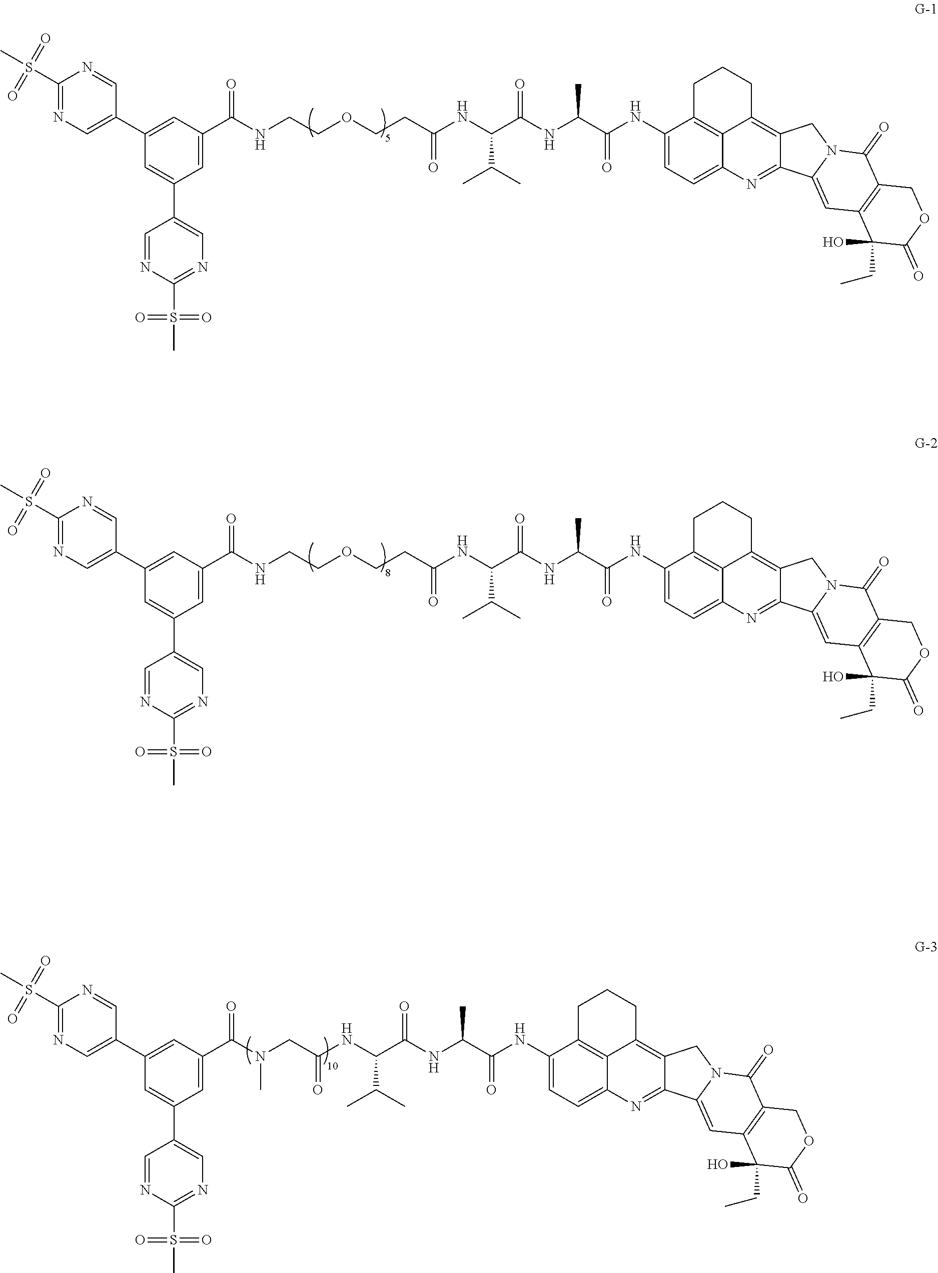

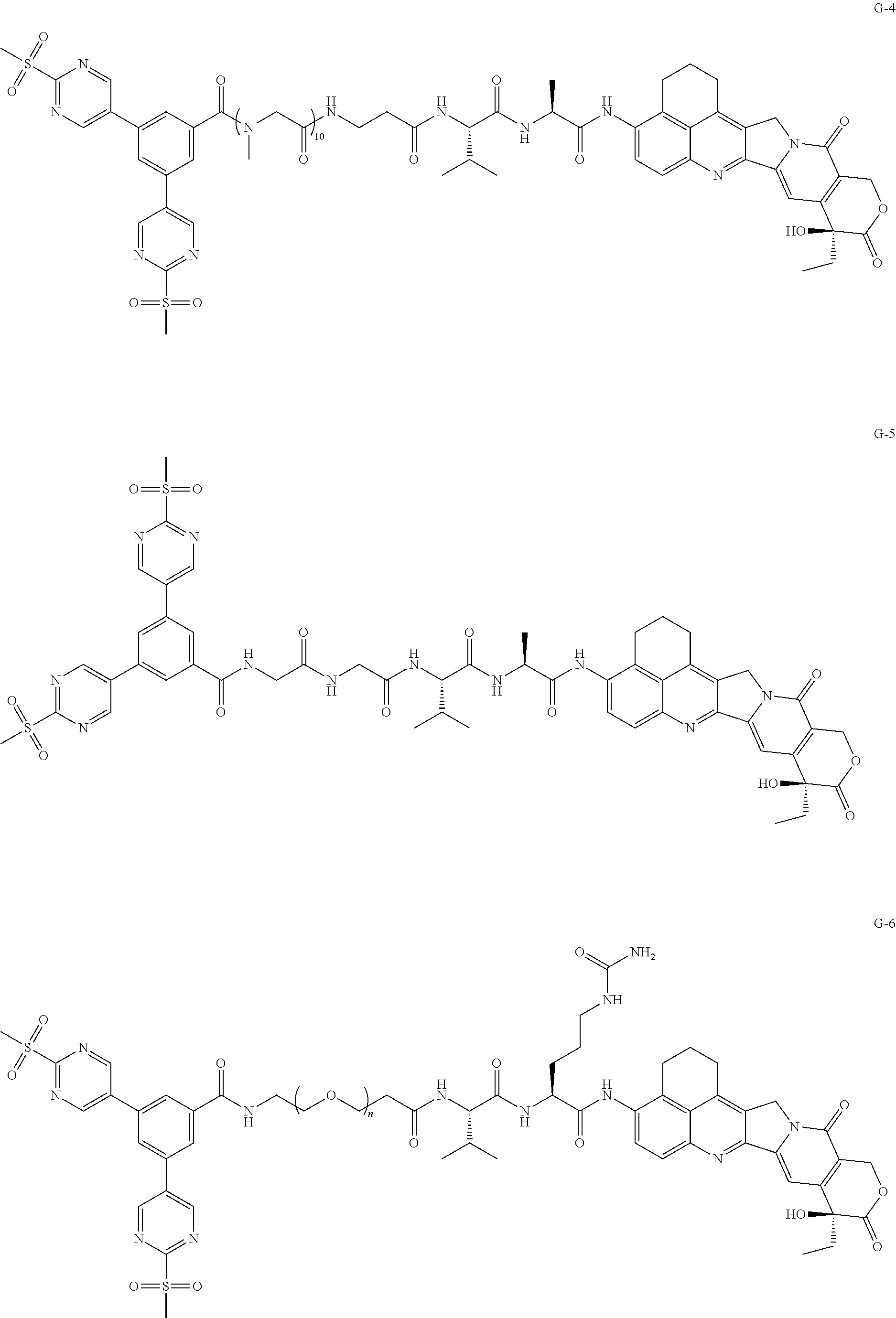

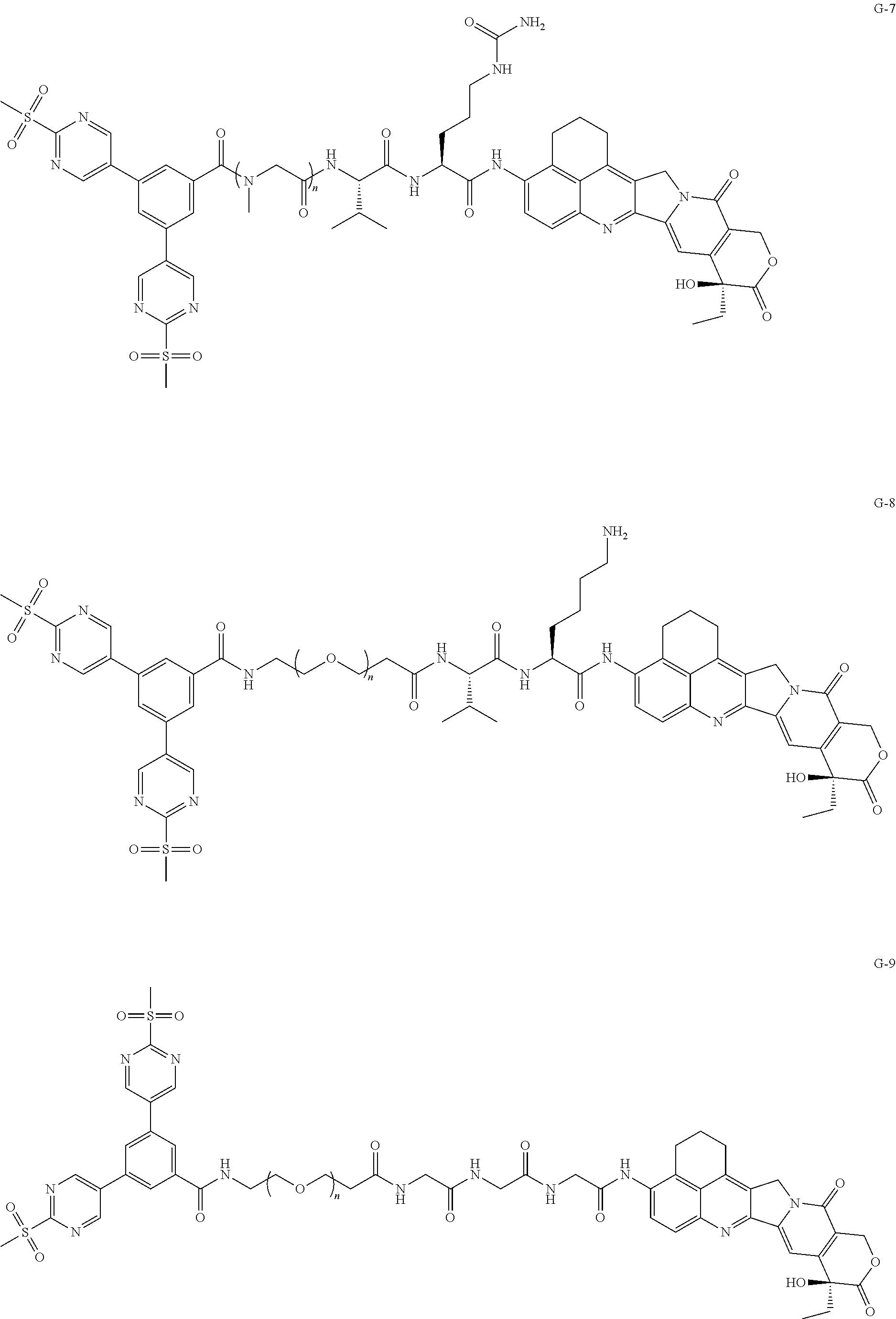

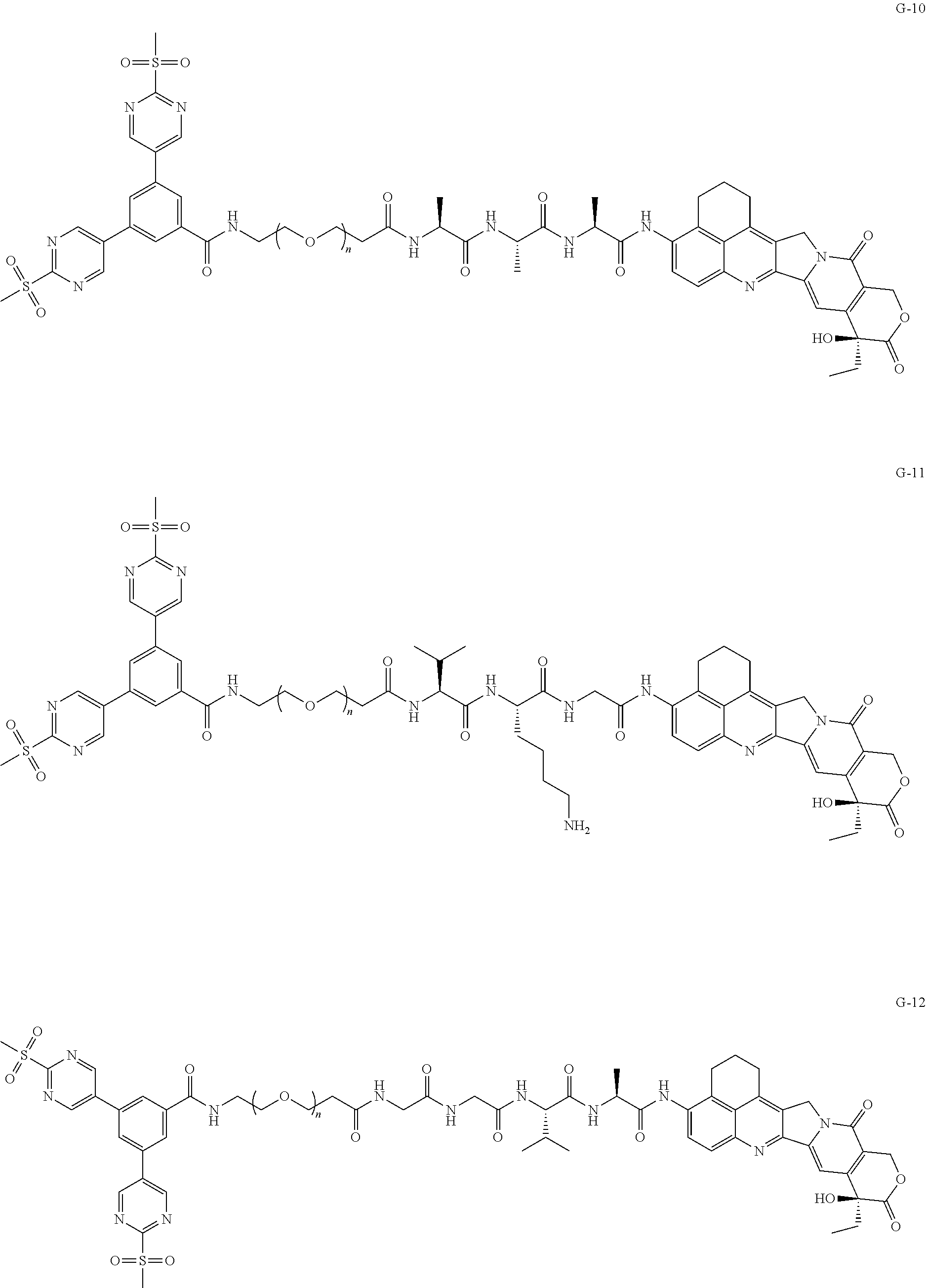

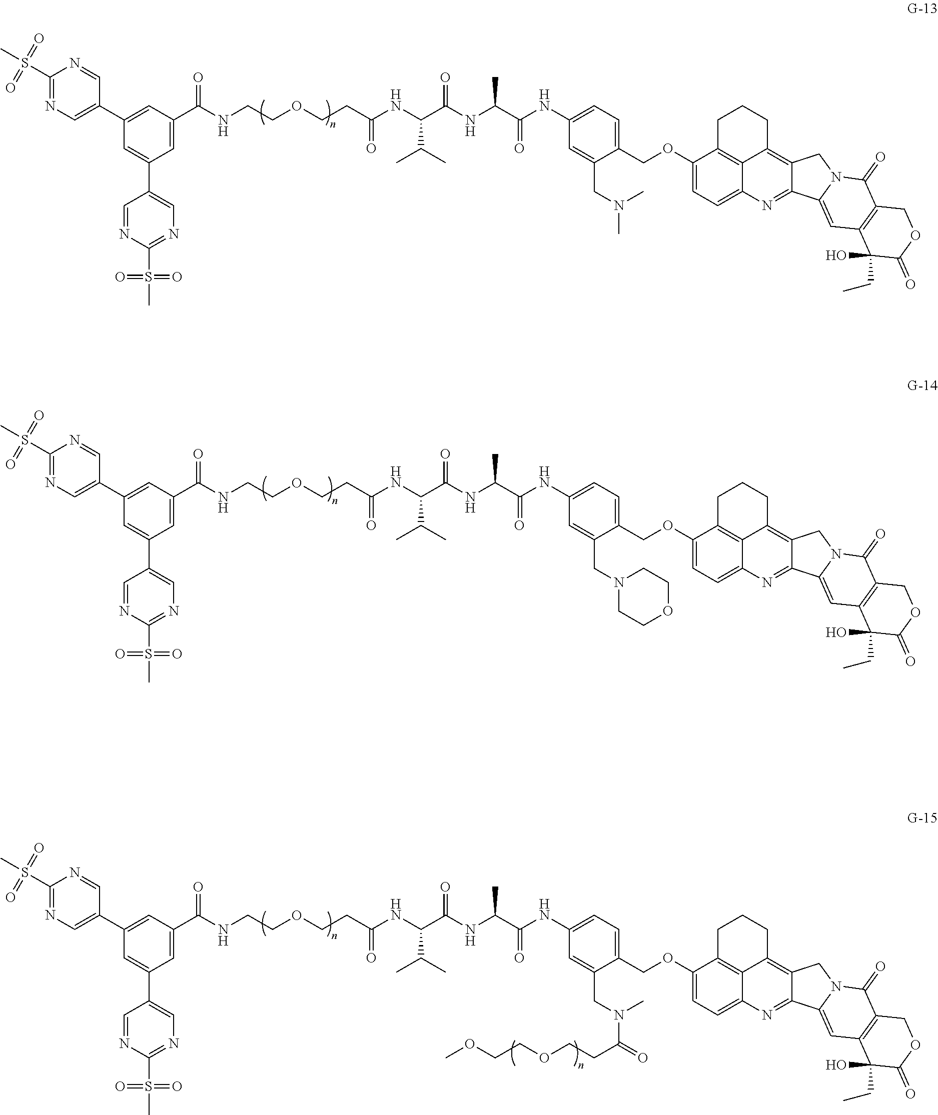

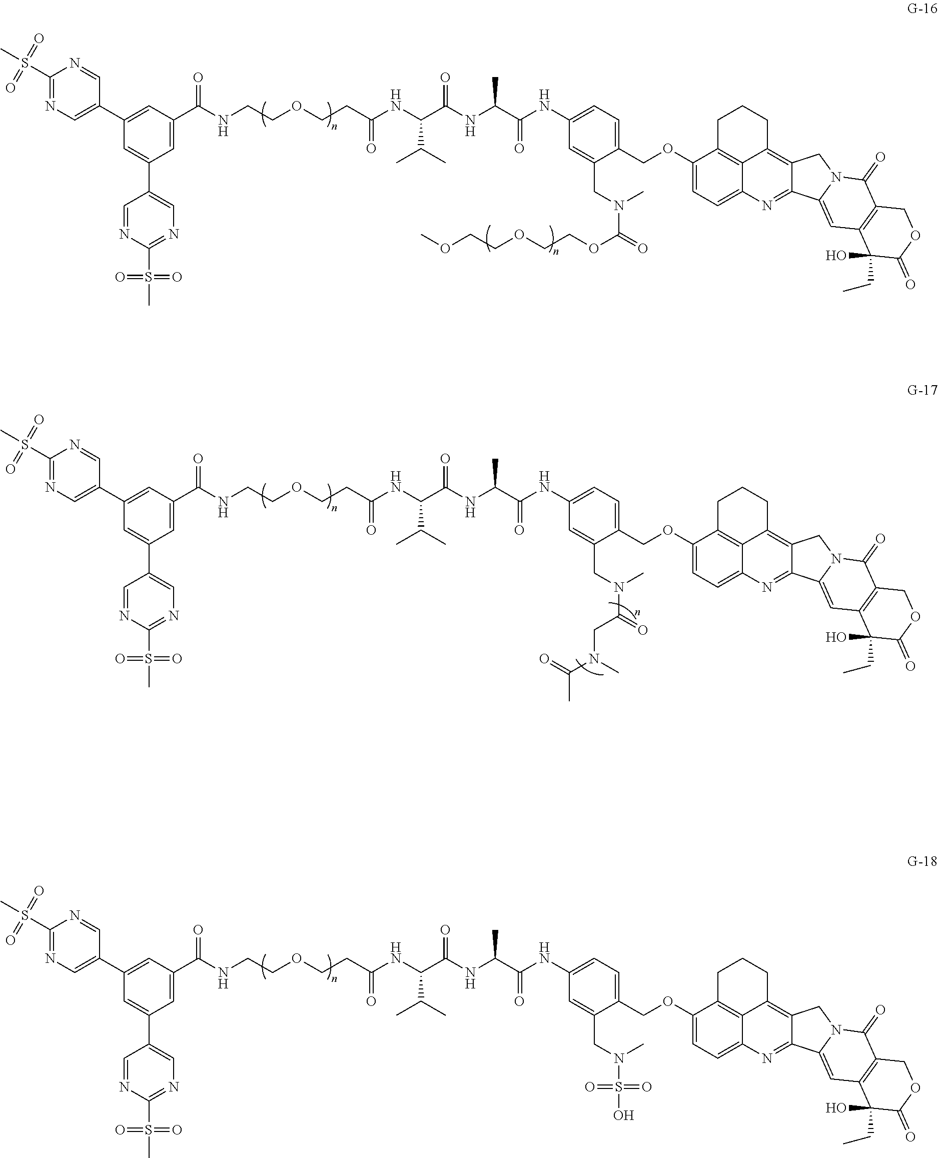

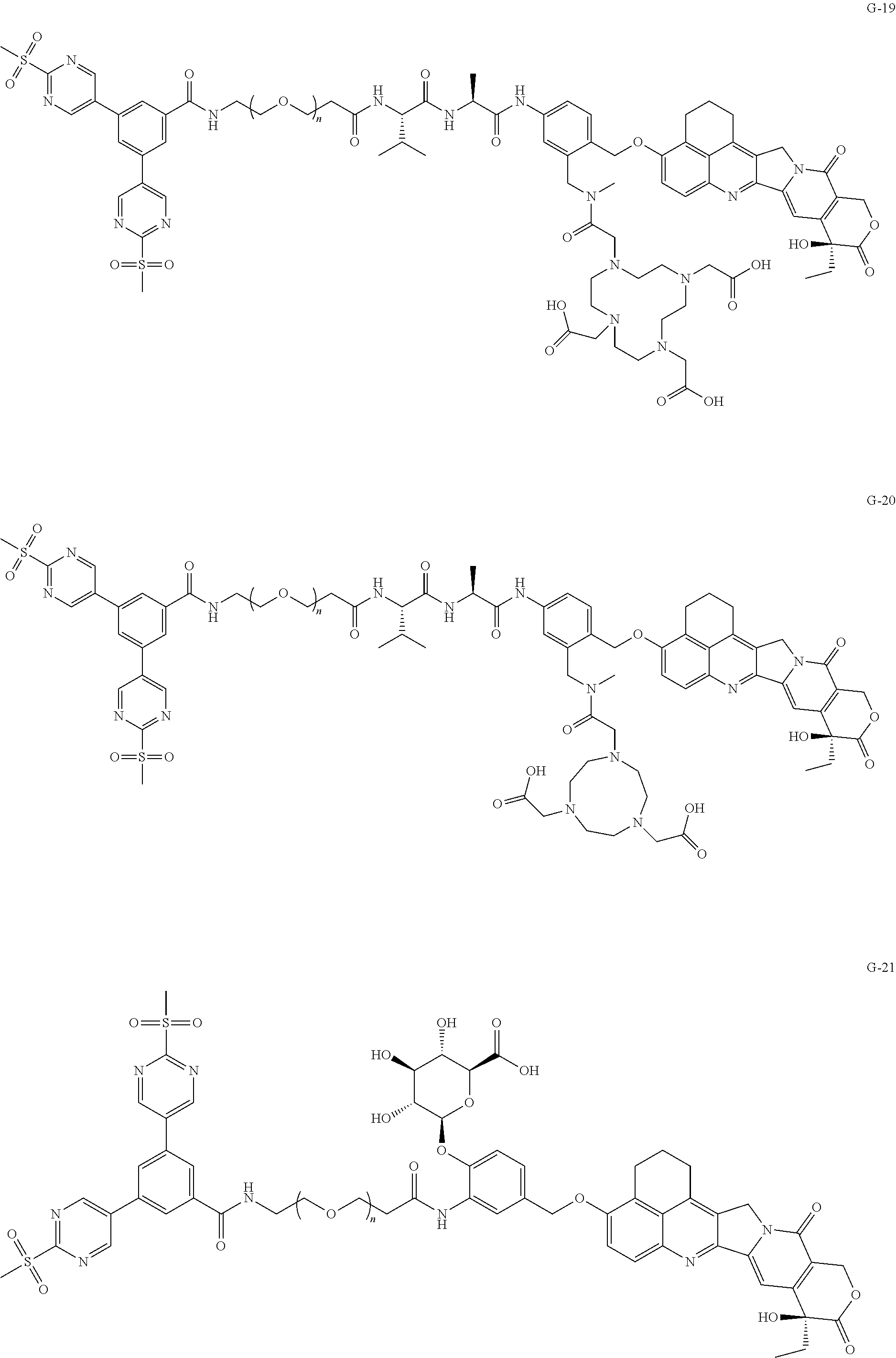

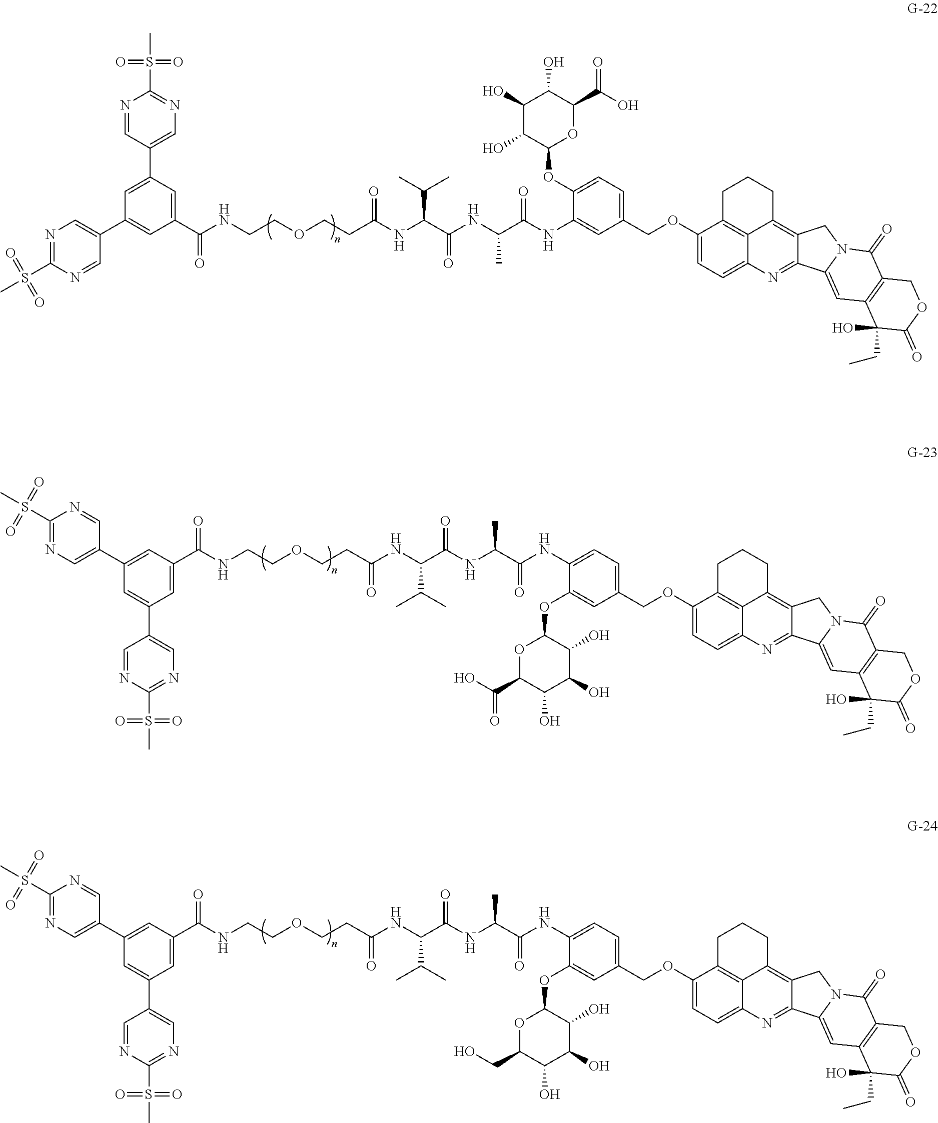

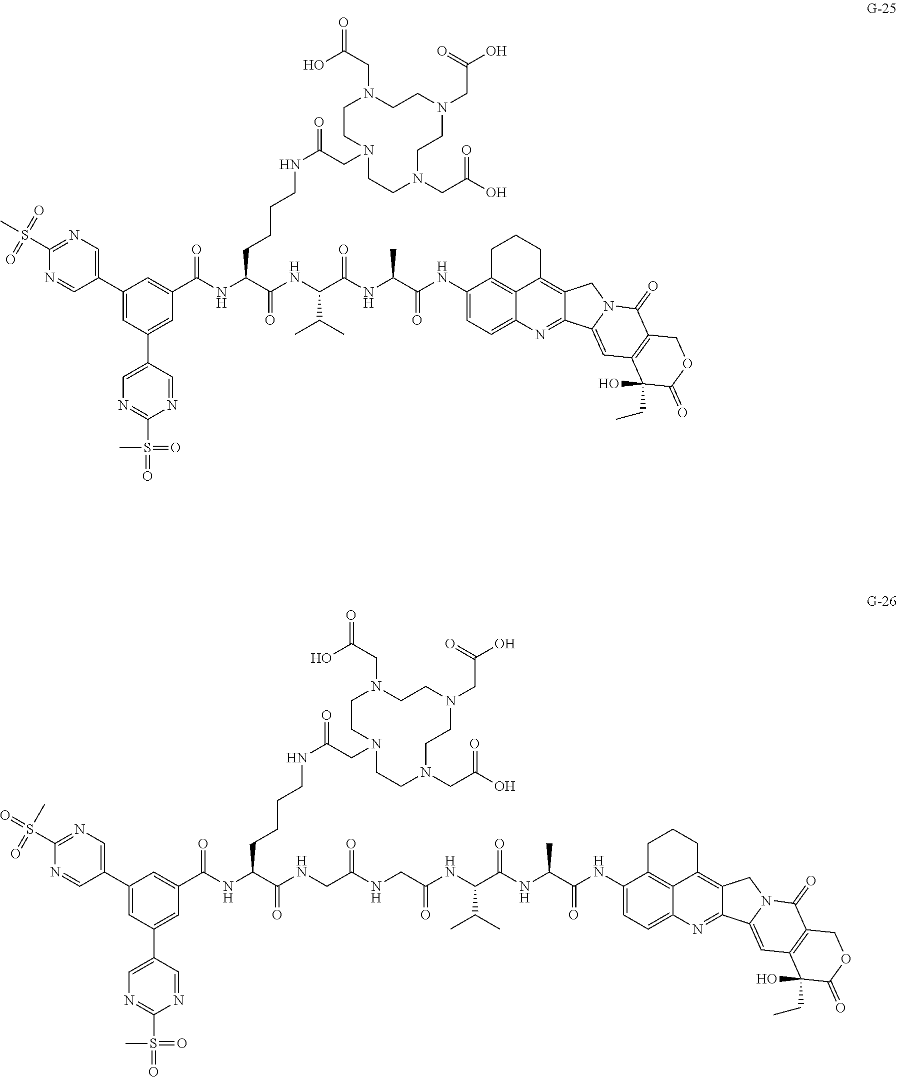

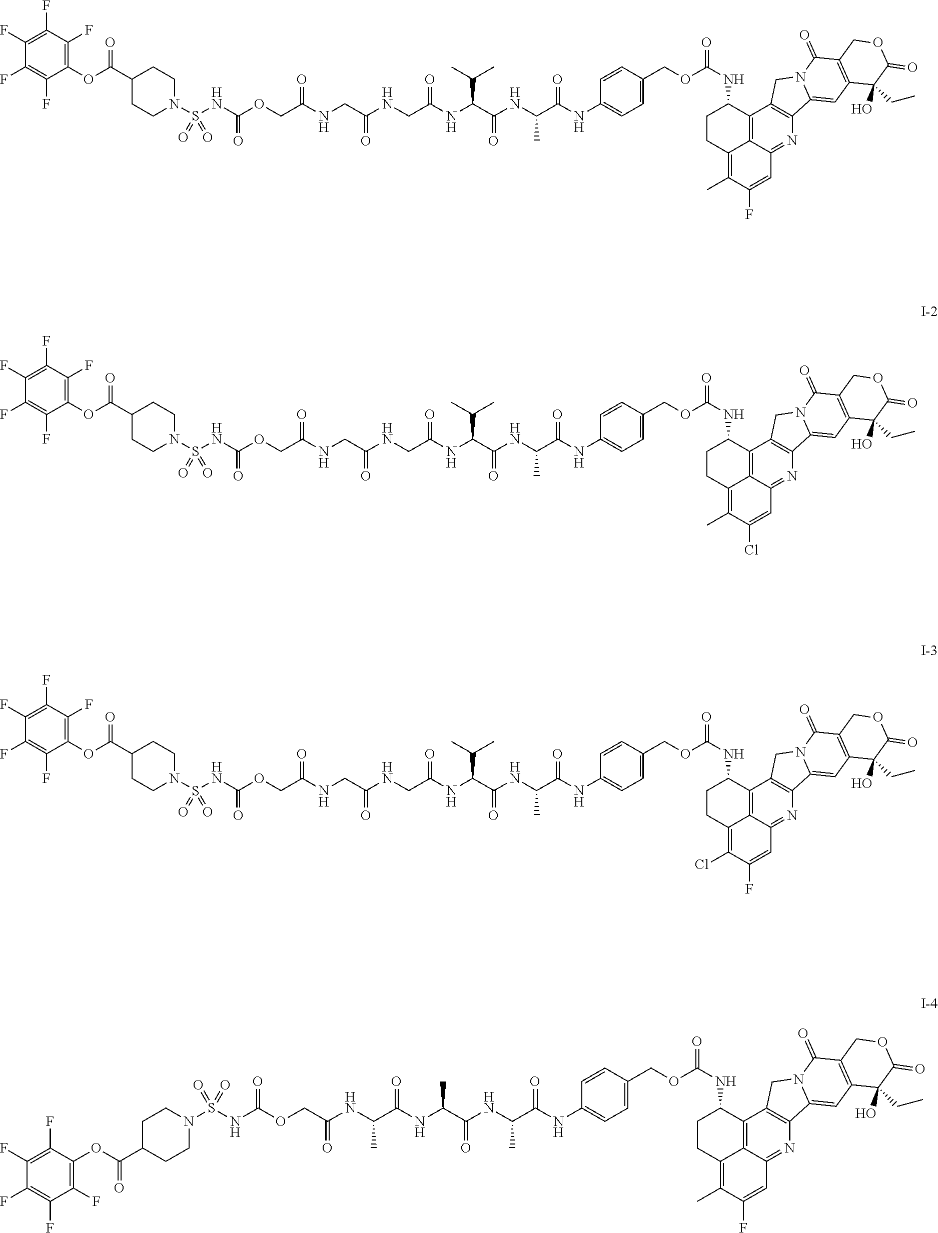

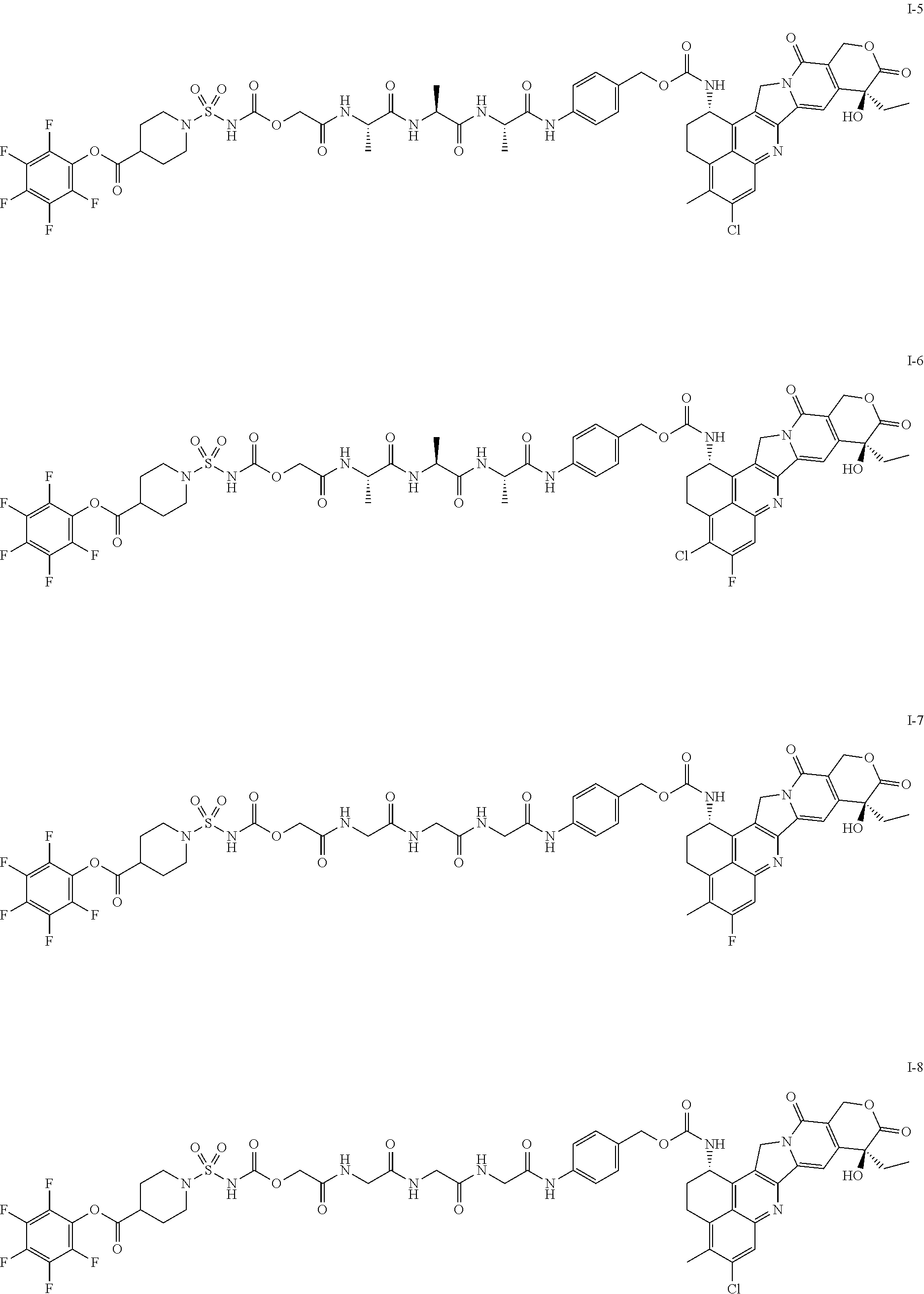

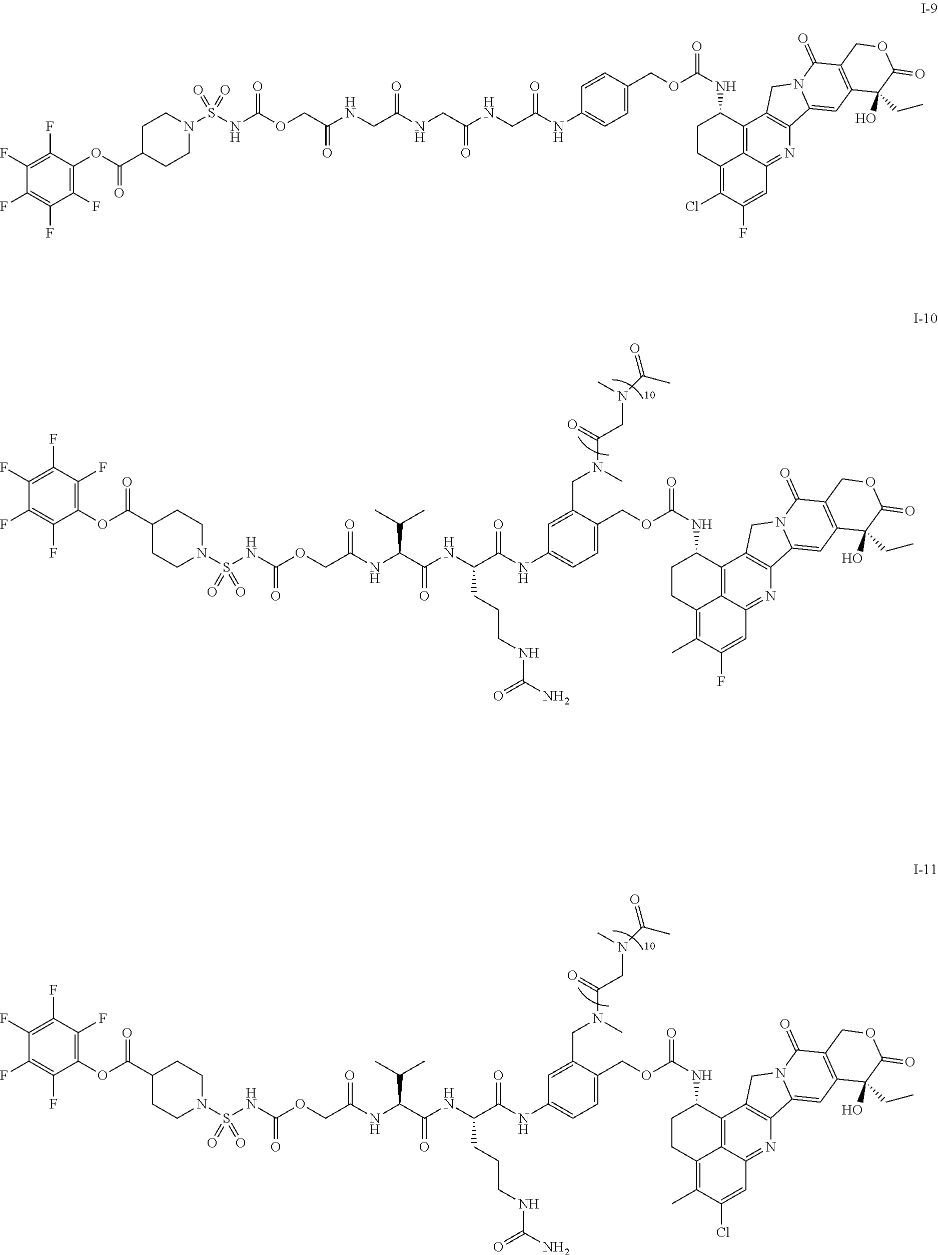

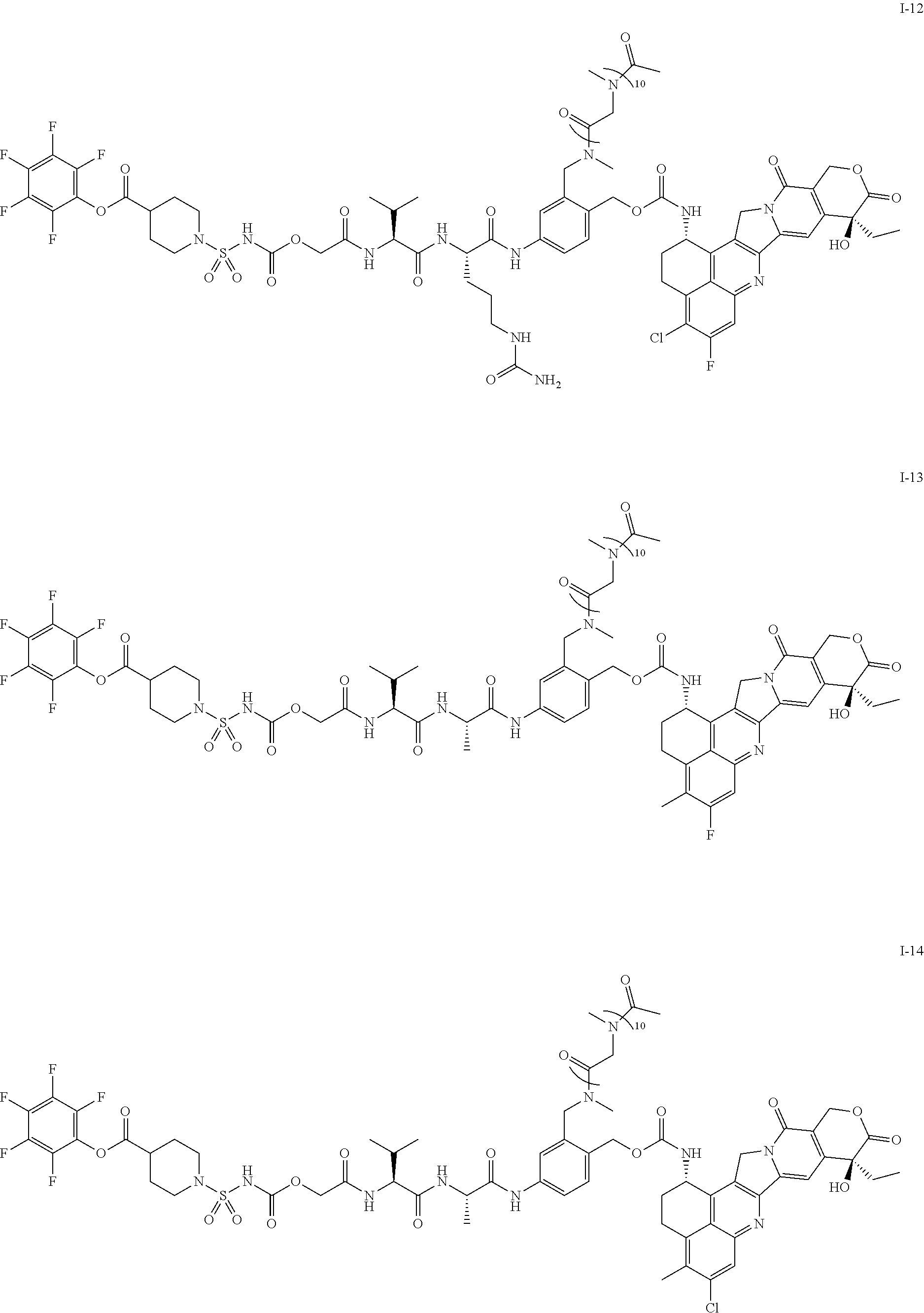

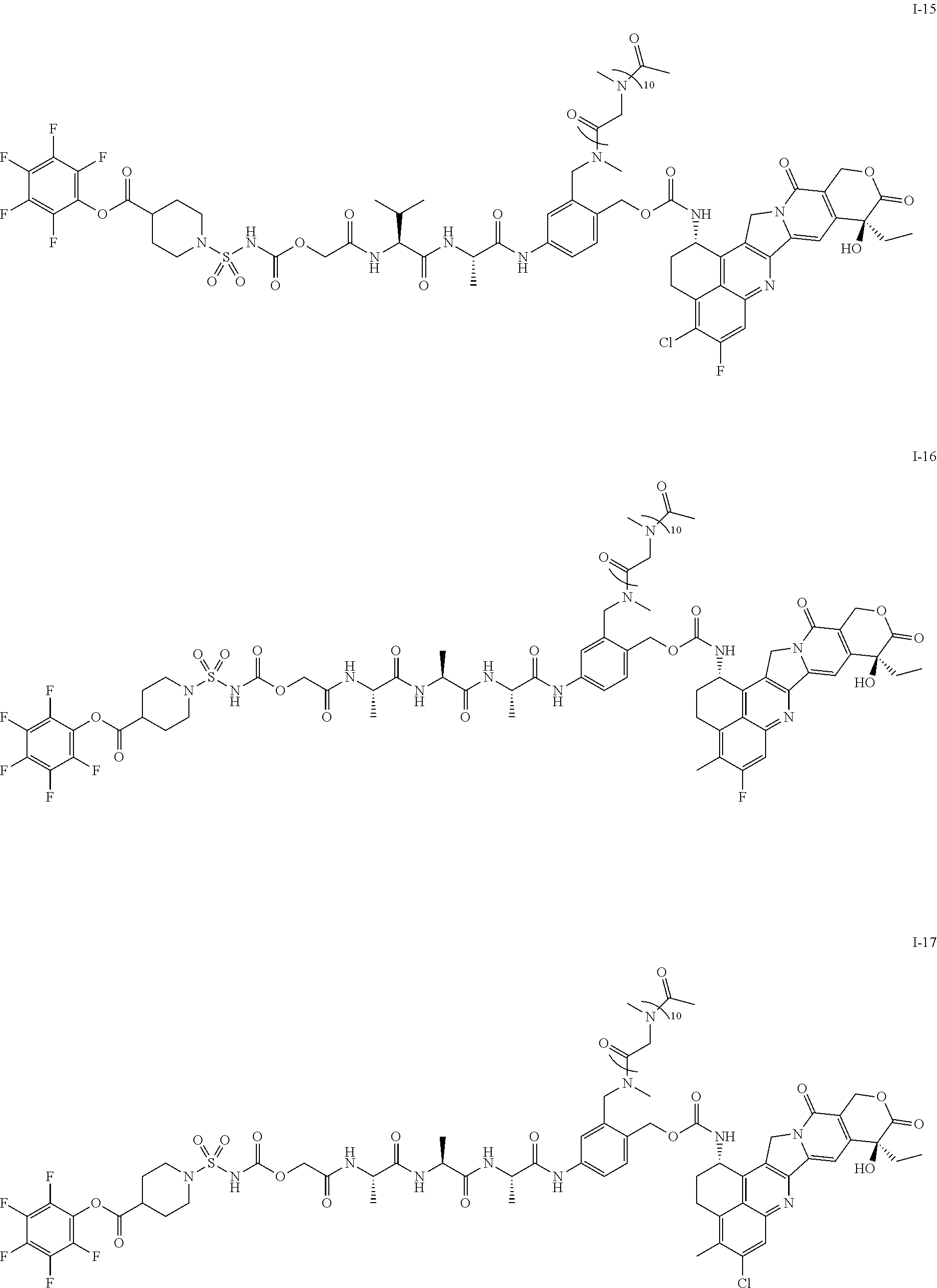

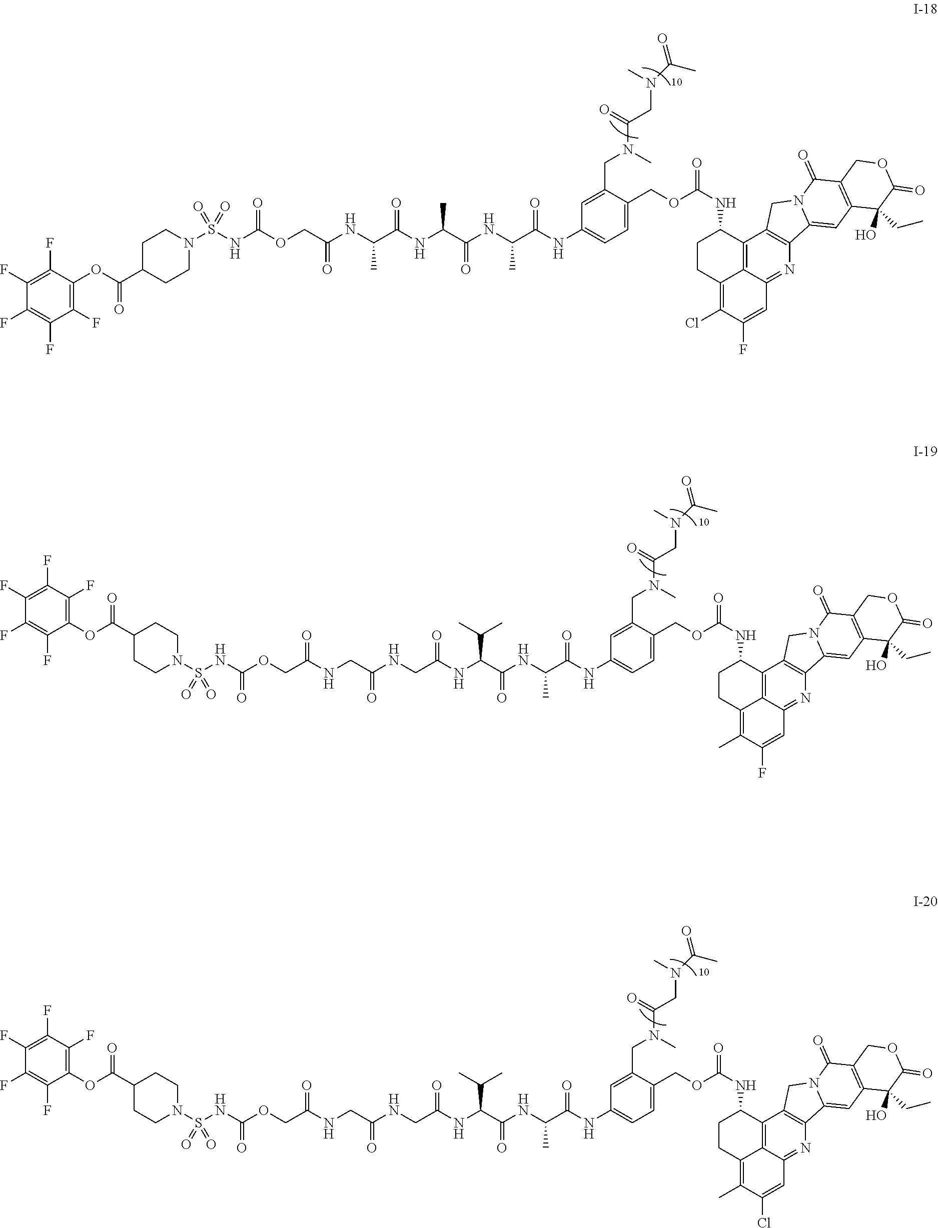

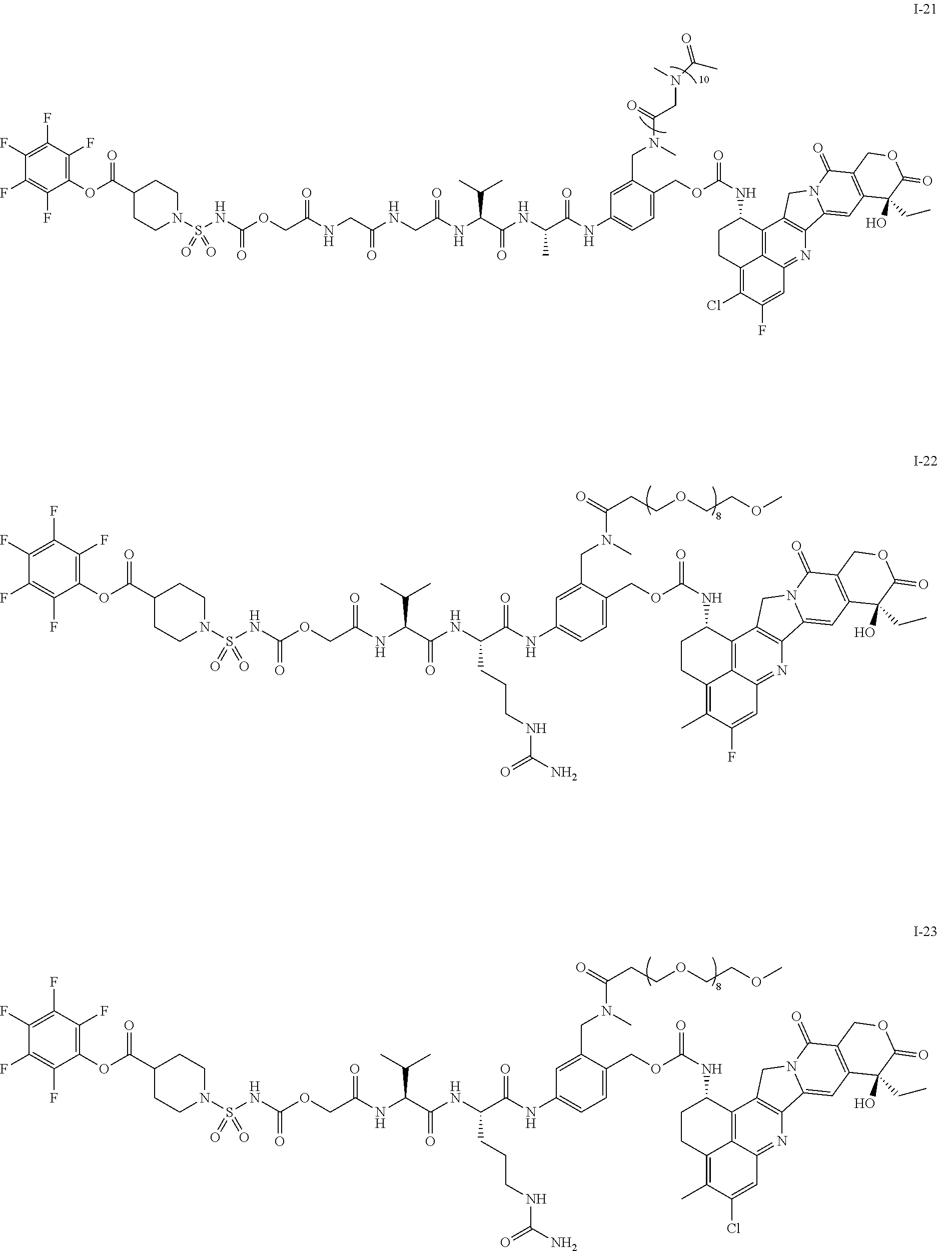

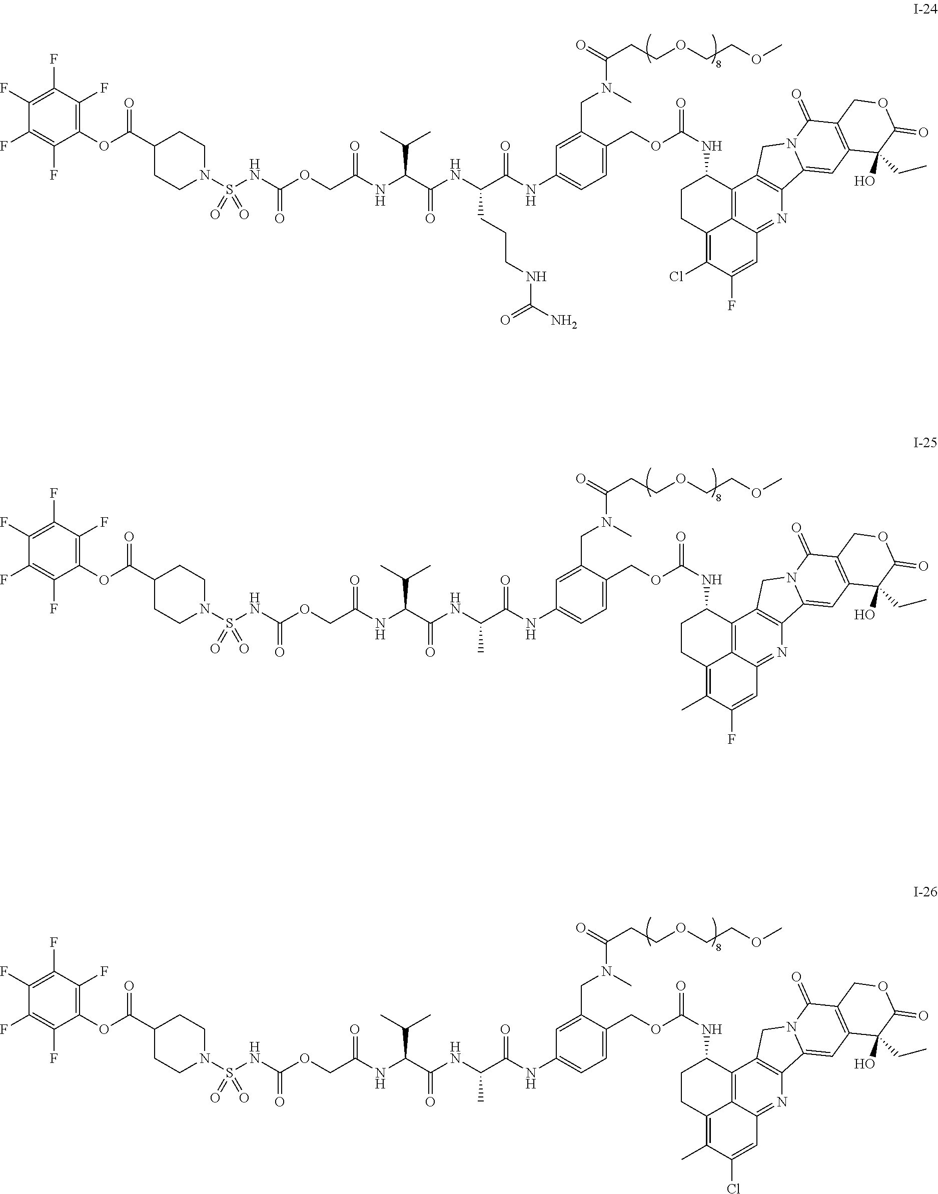

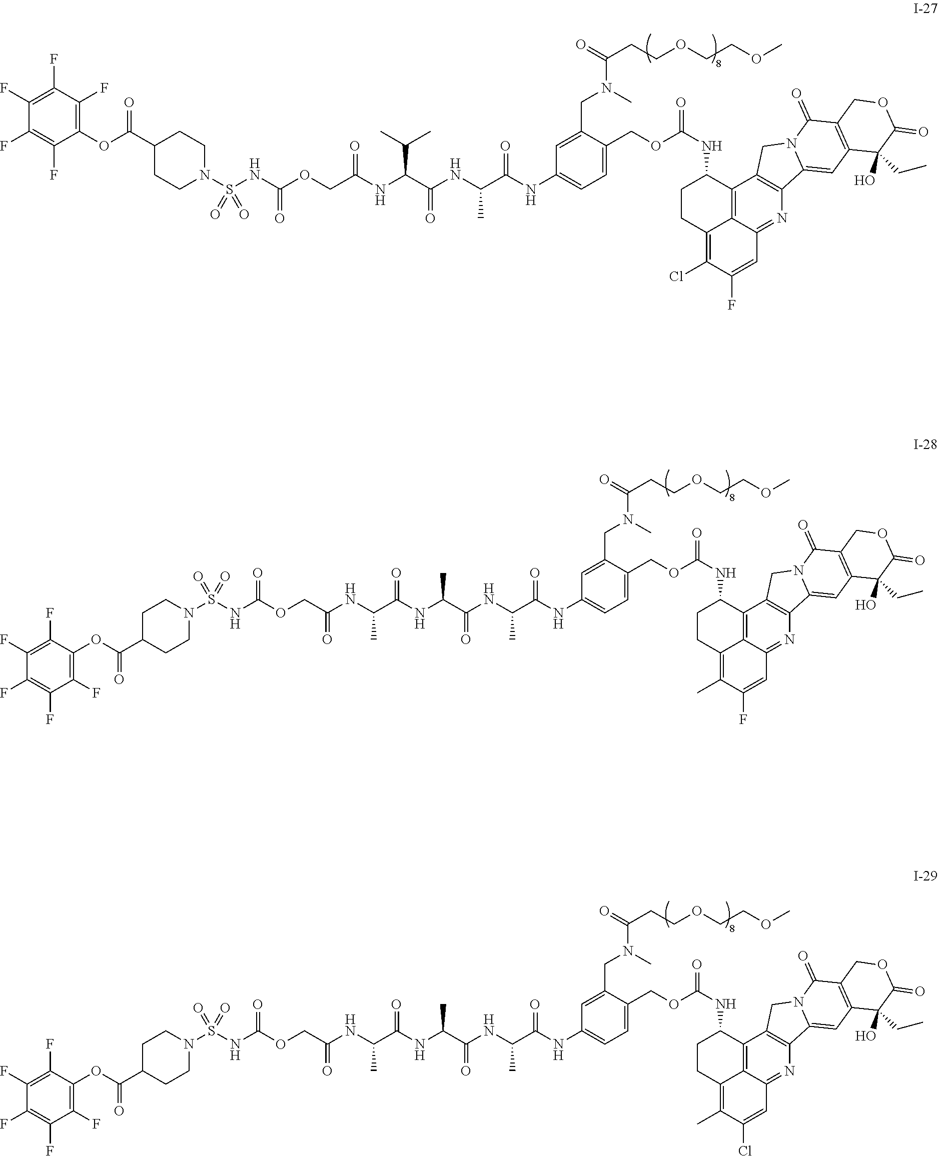

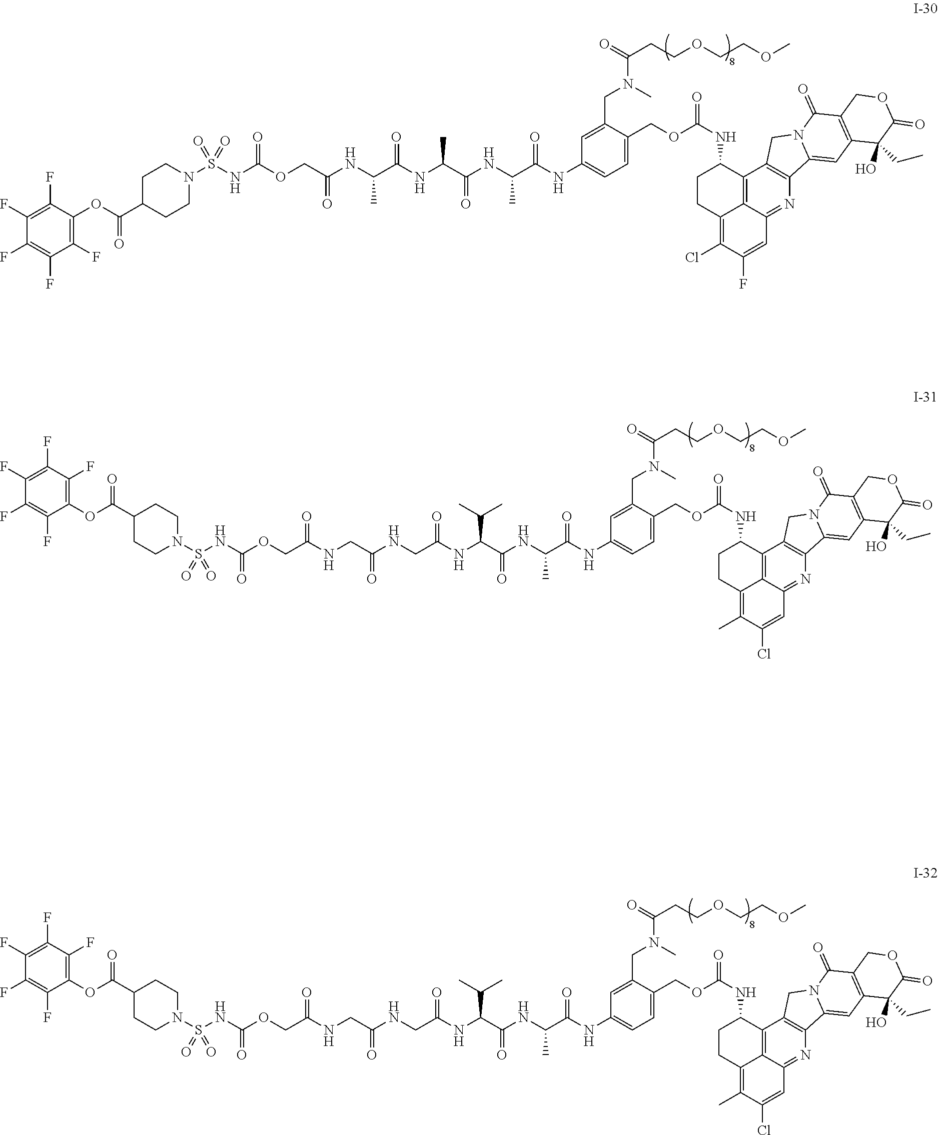

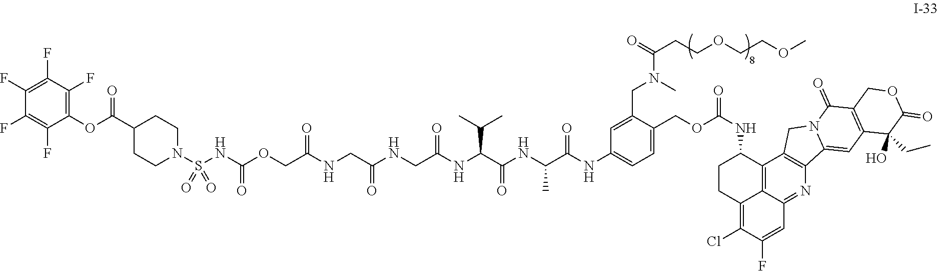

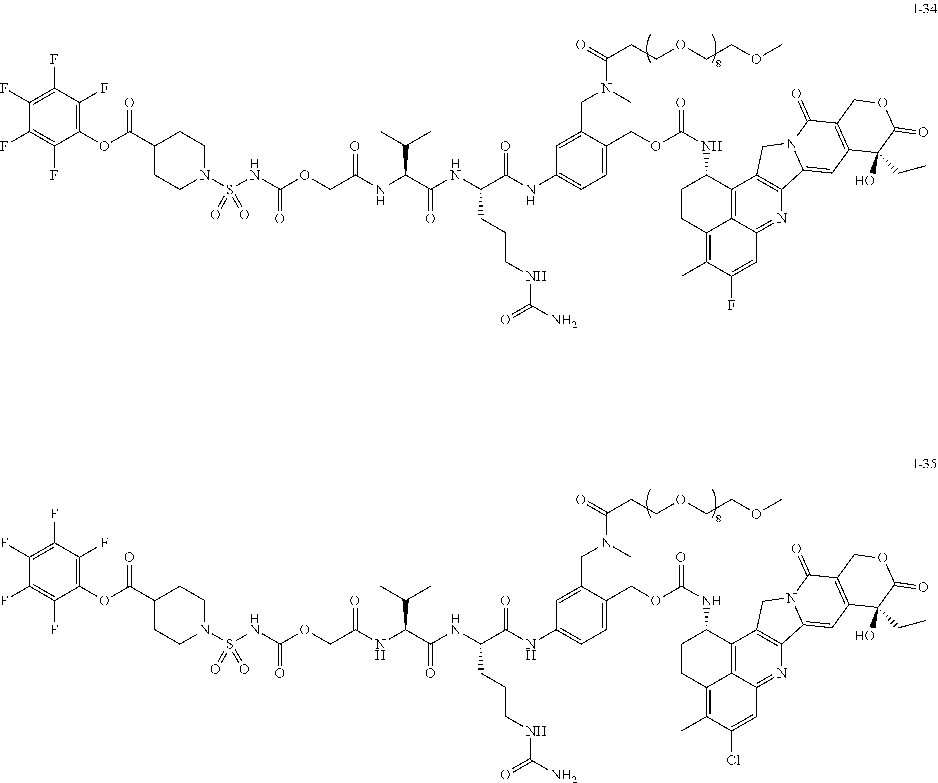

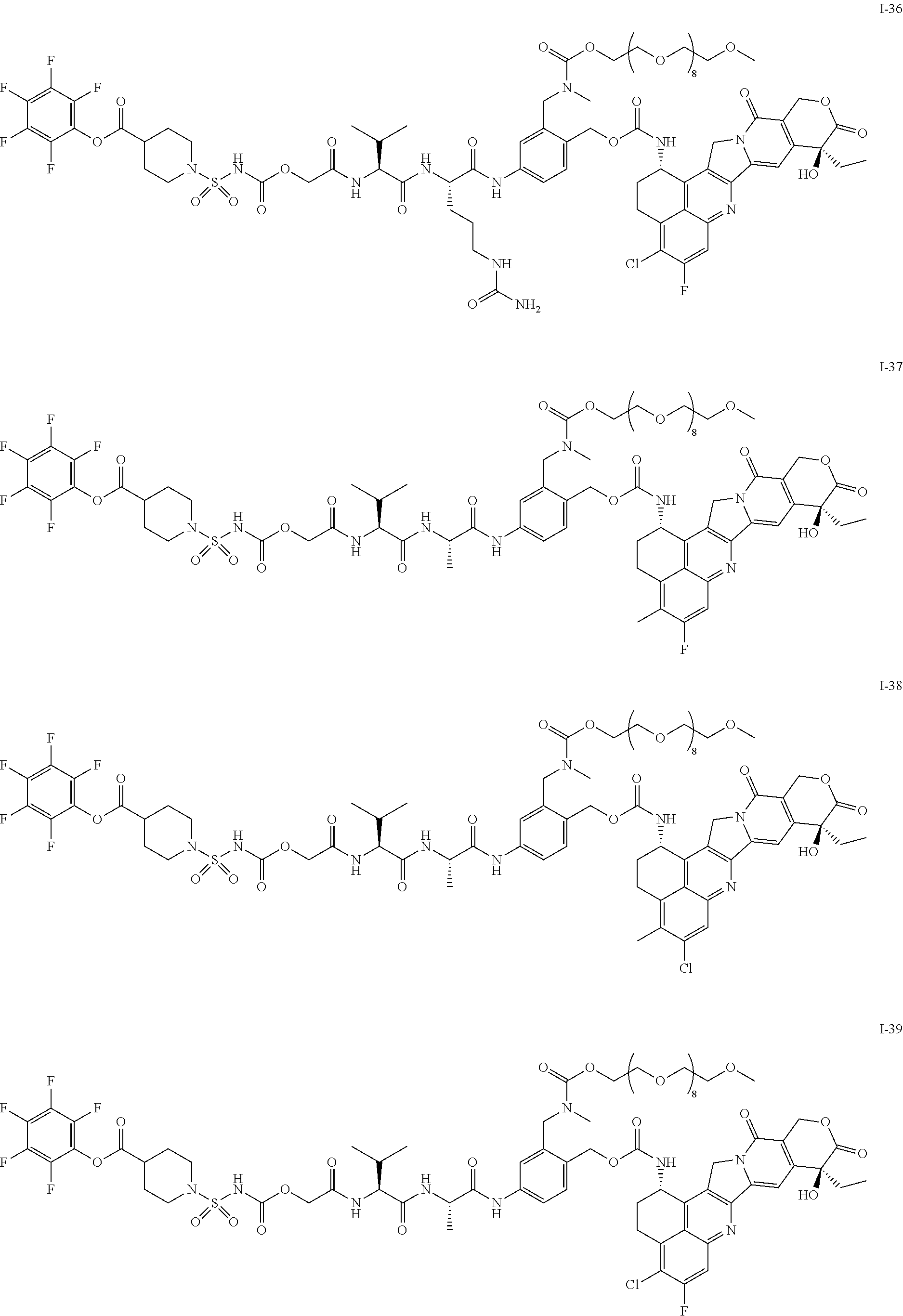

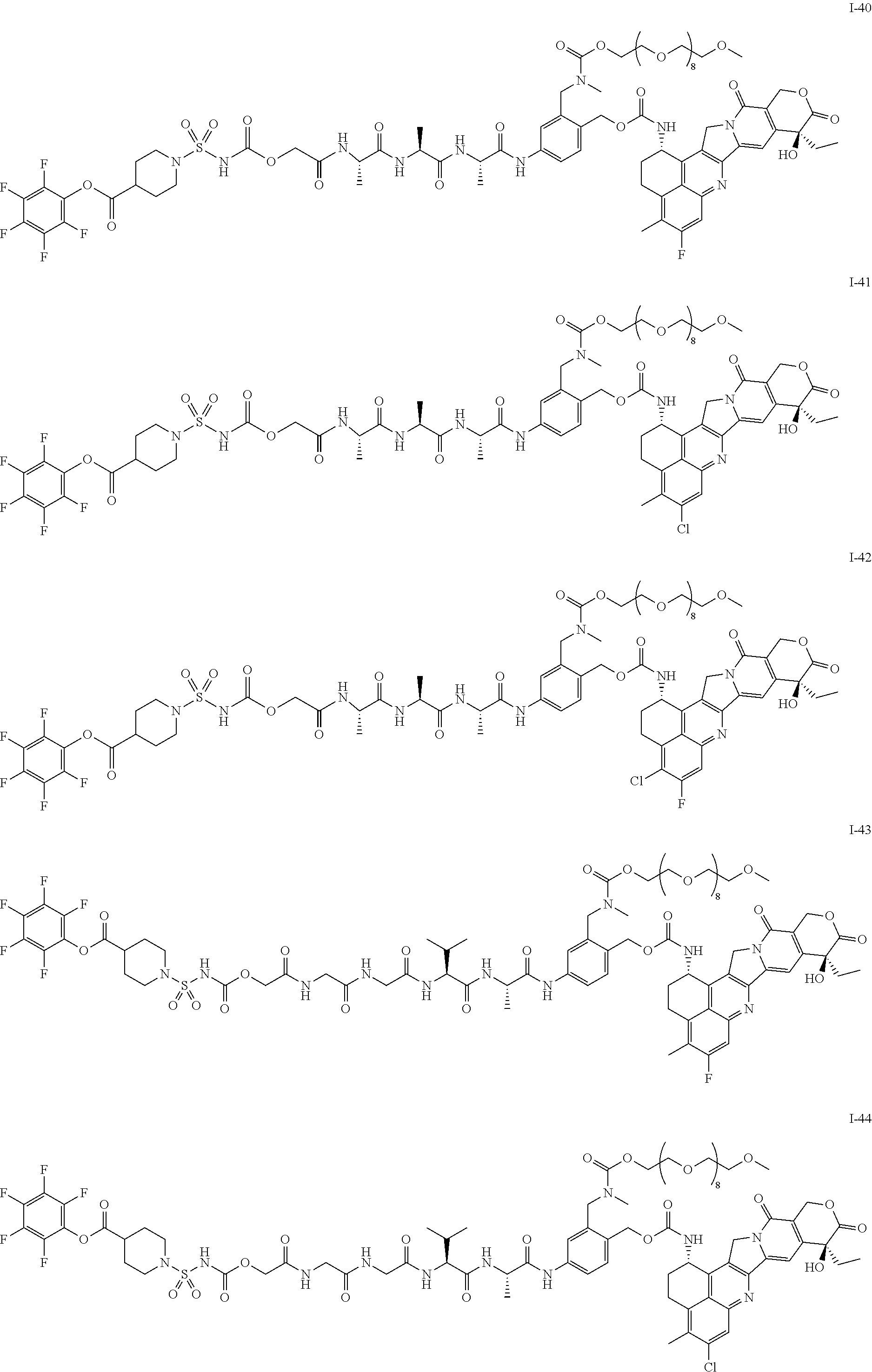

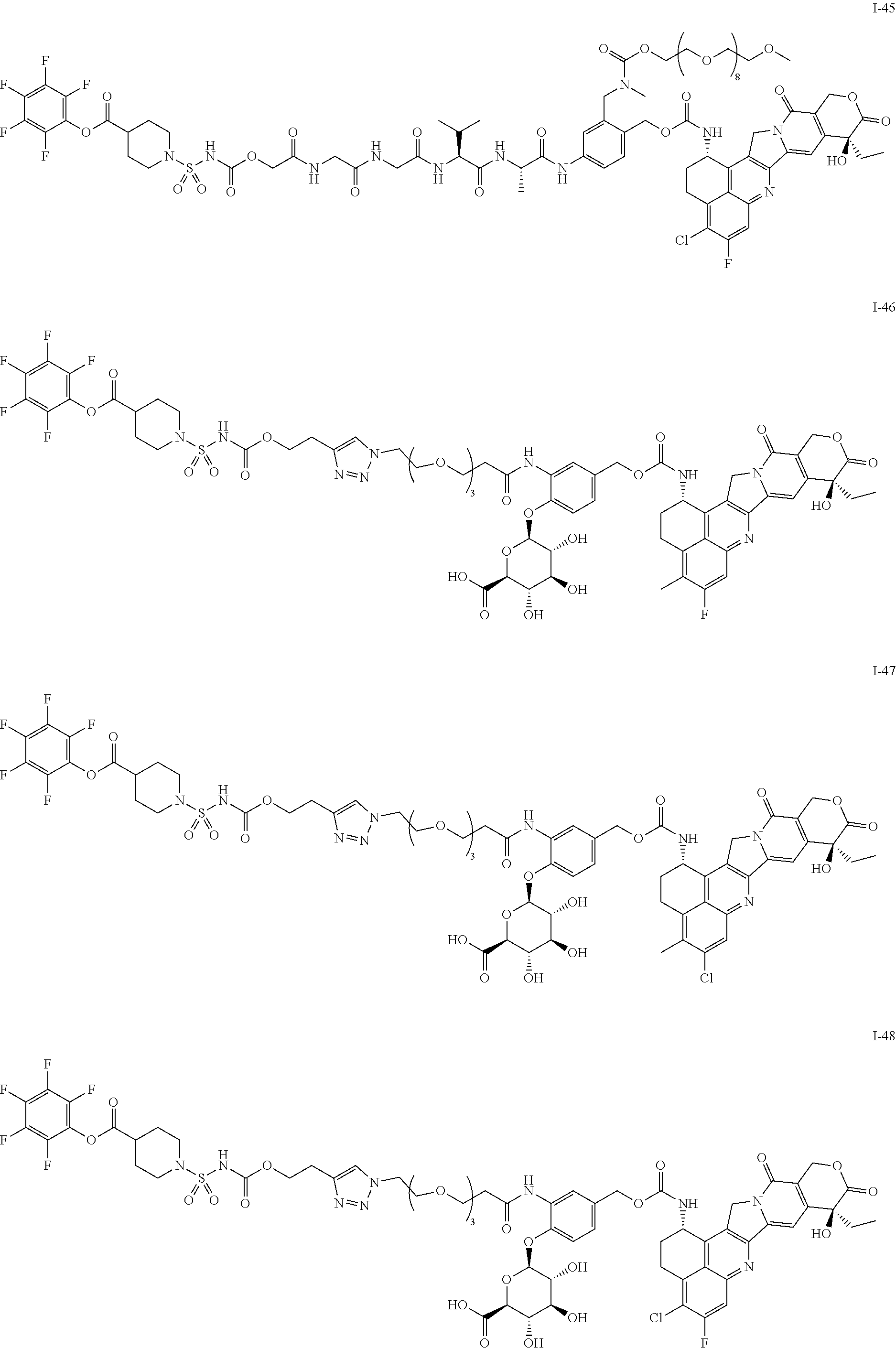

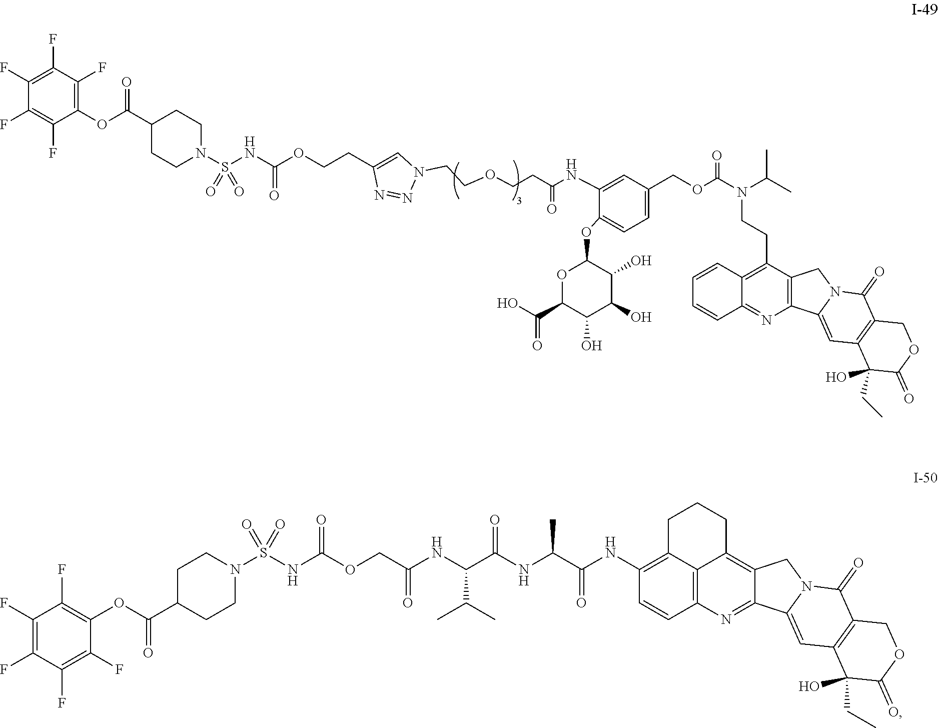

wherein n is an integer selected from 1 to 20, such as an integer of 1 to 12, 3 to 12, for example, n is 1, 2, 3, 4, 5, 6, 7, 8, 9, 10, 11, 12, 13, 14 or 15.

In some embodiments, the present application provides a compound or pharmaceutically acceptable salt thereof as shown below:

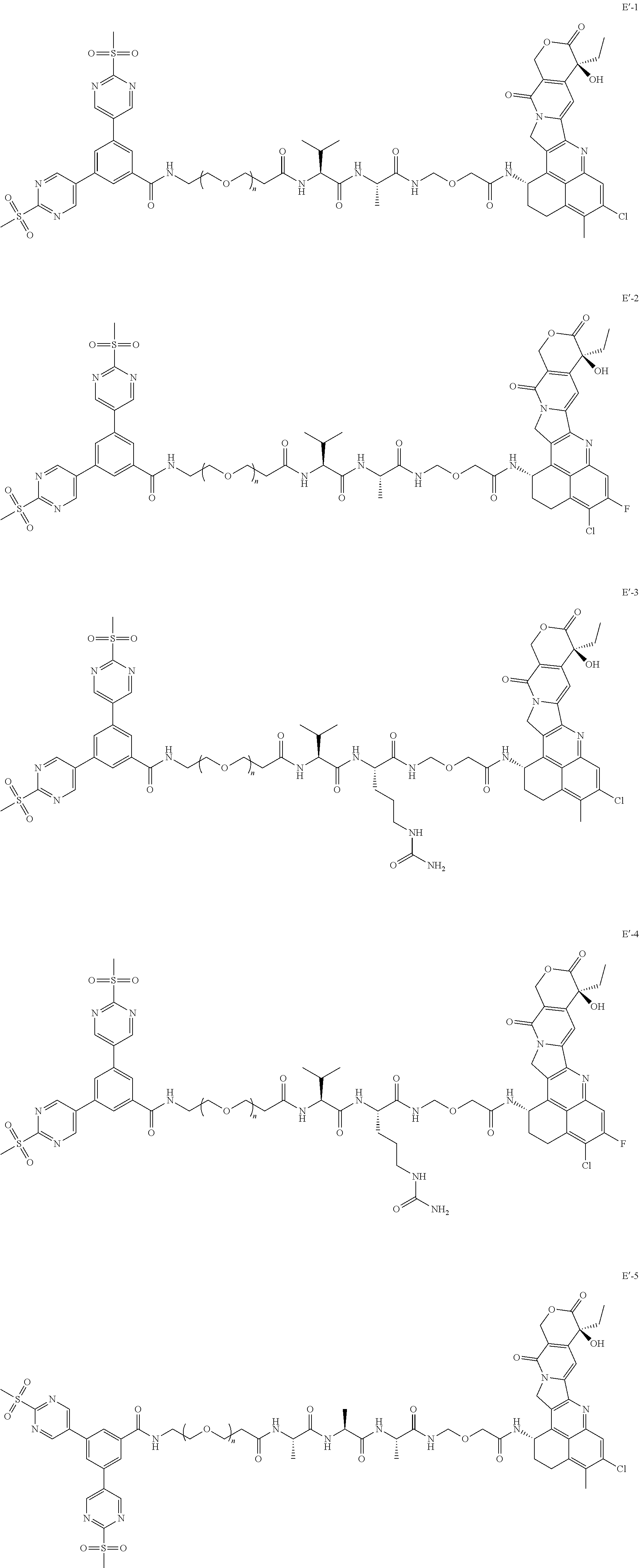

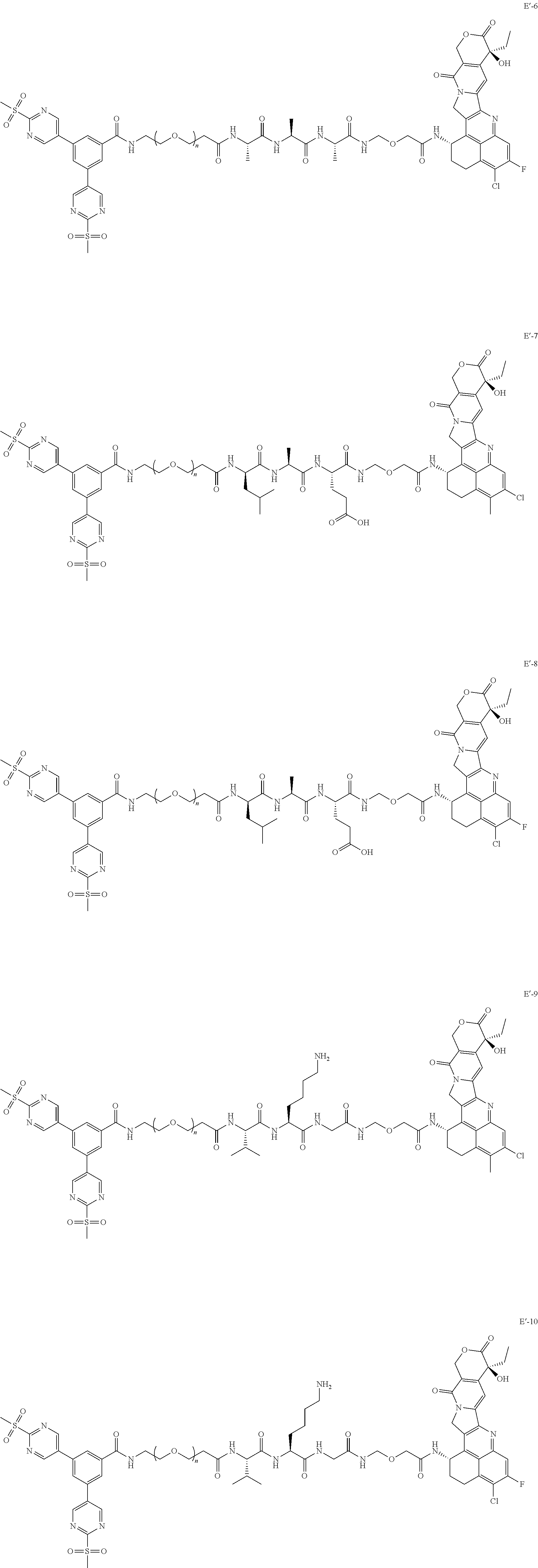

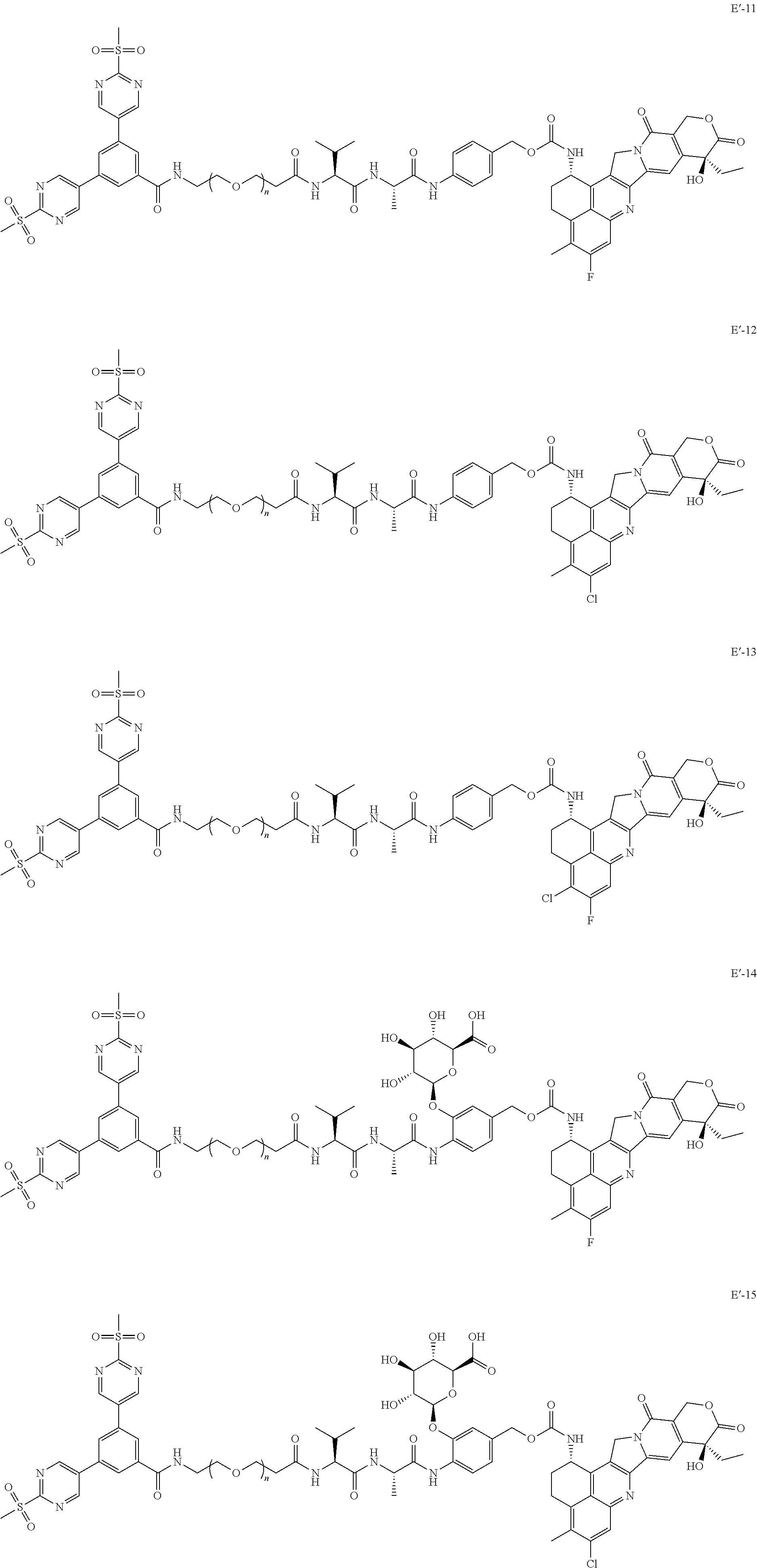

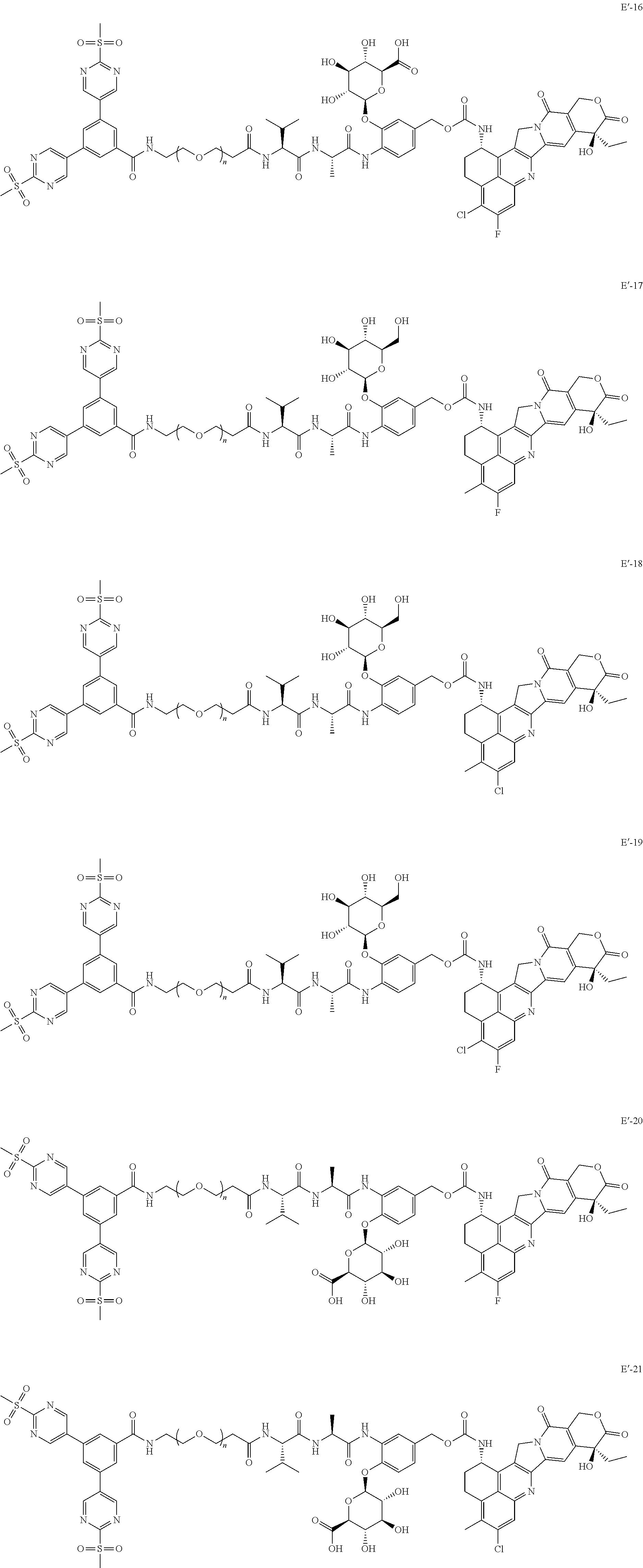

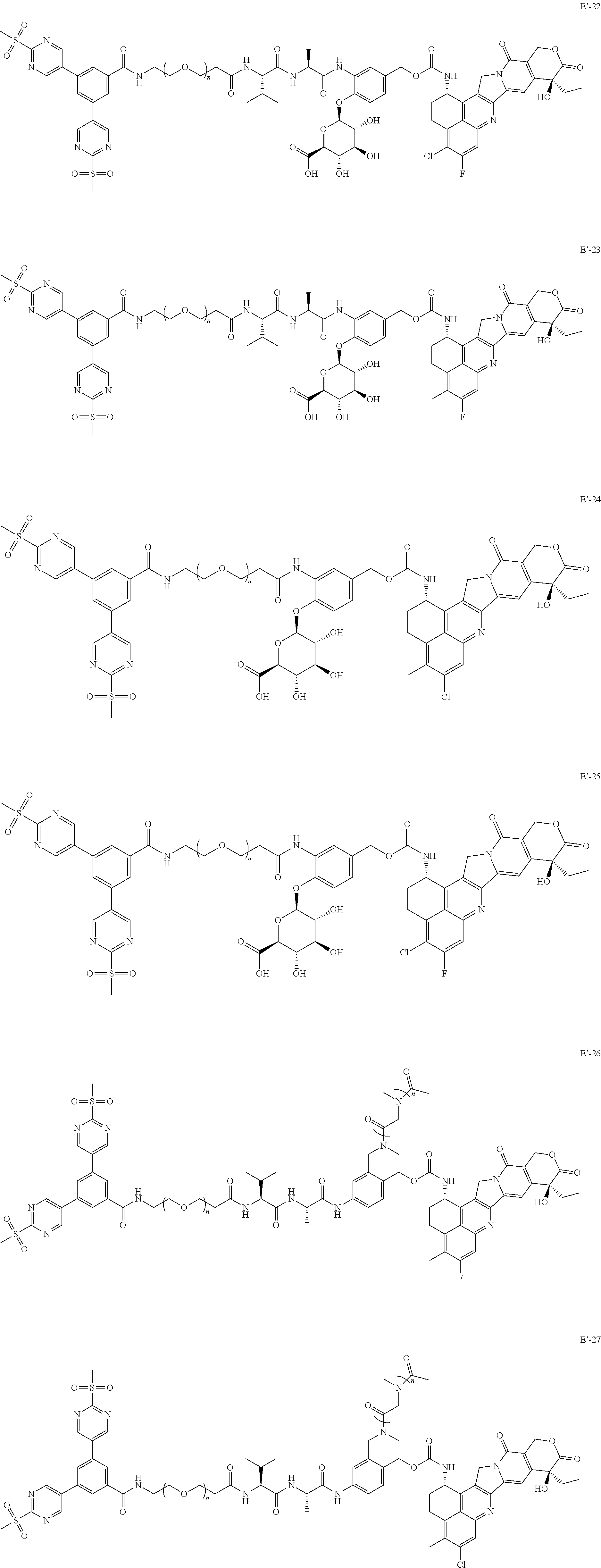

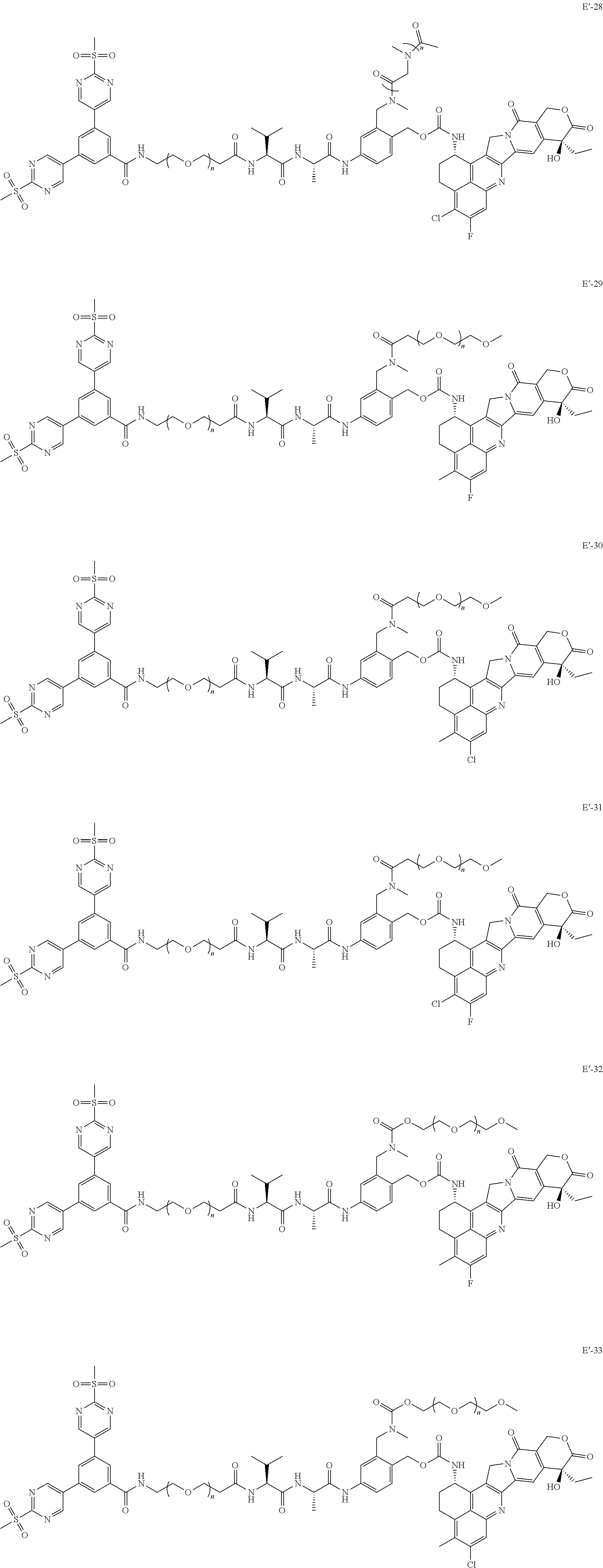

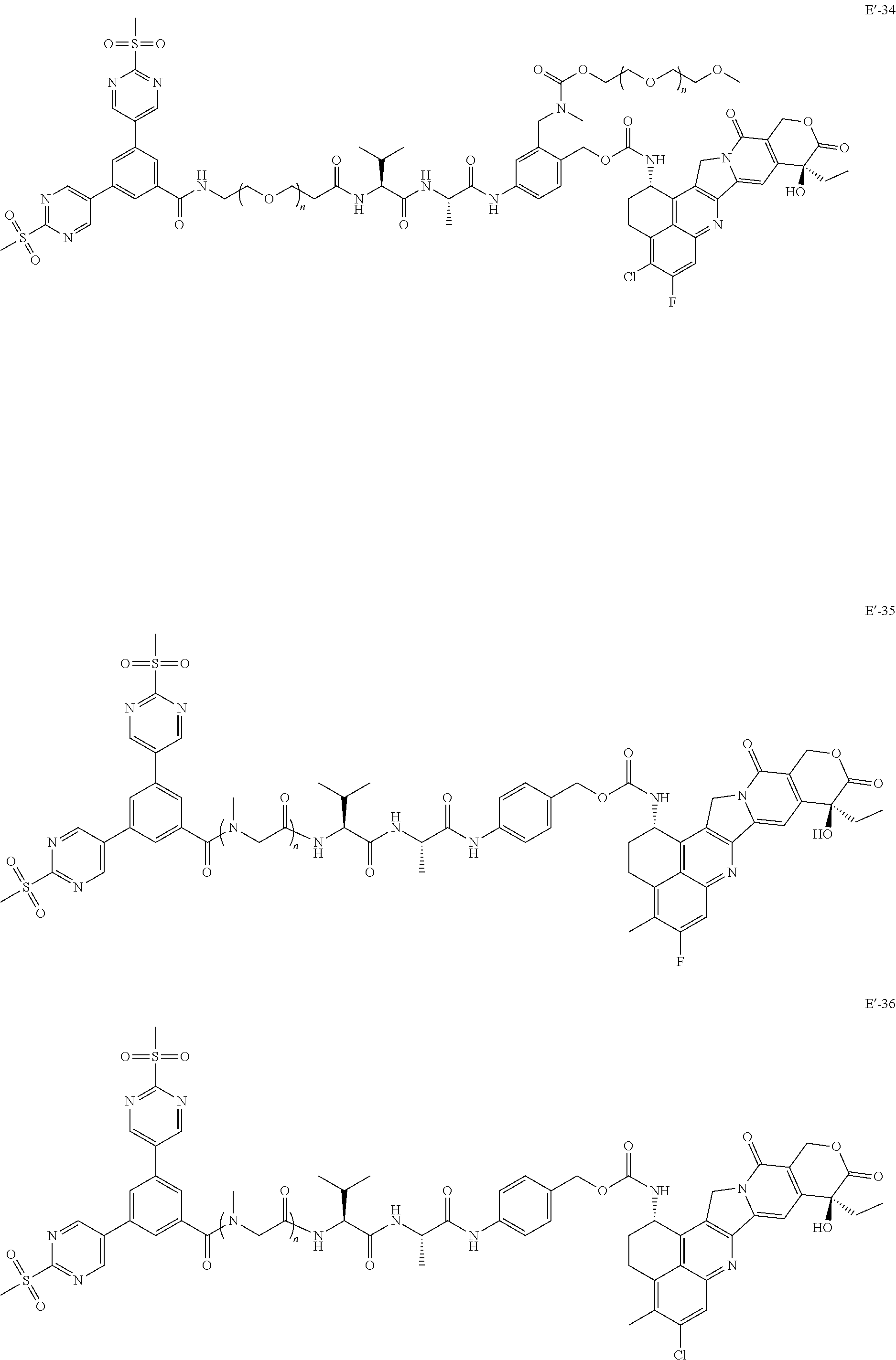

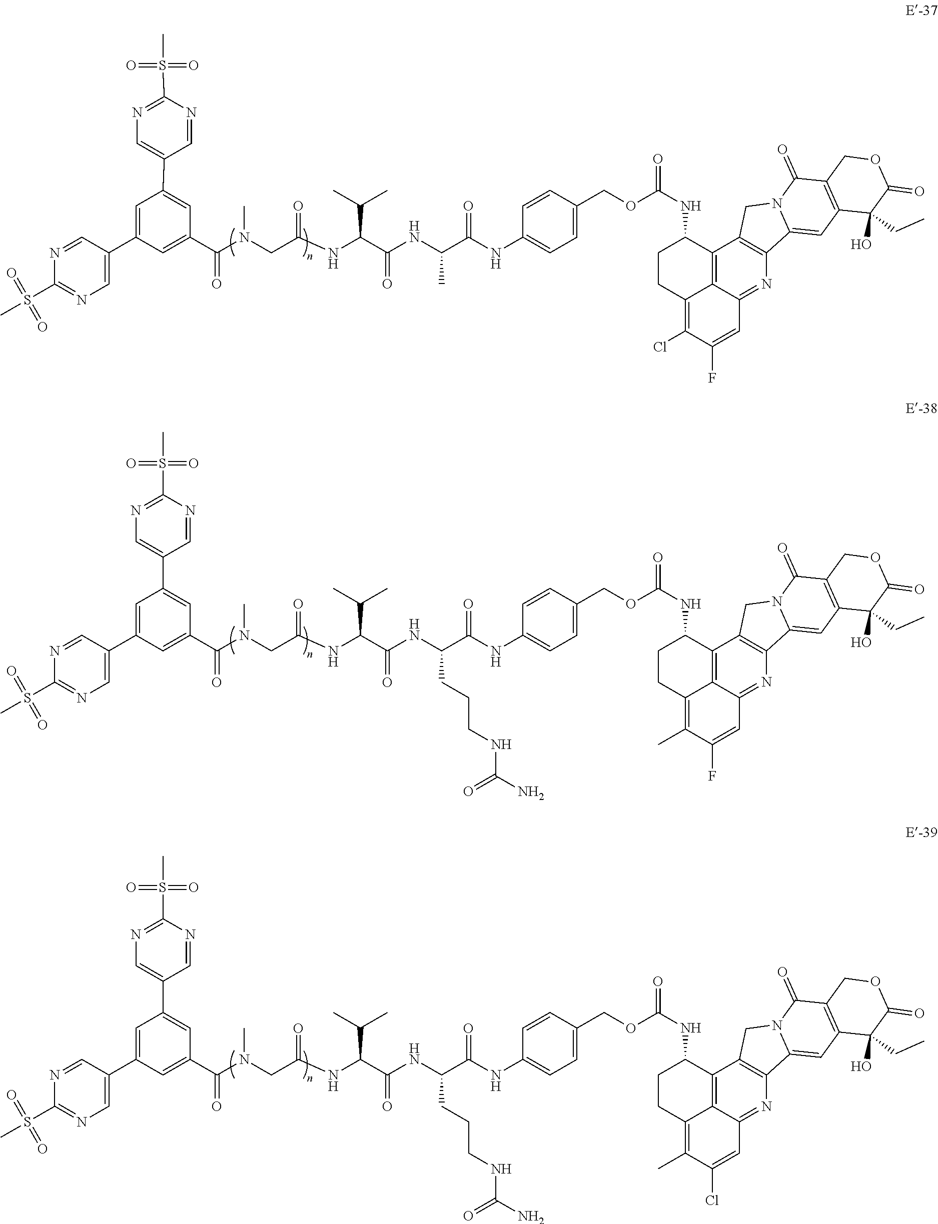

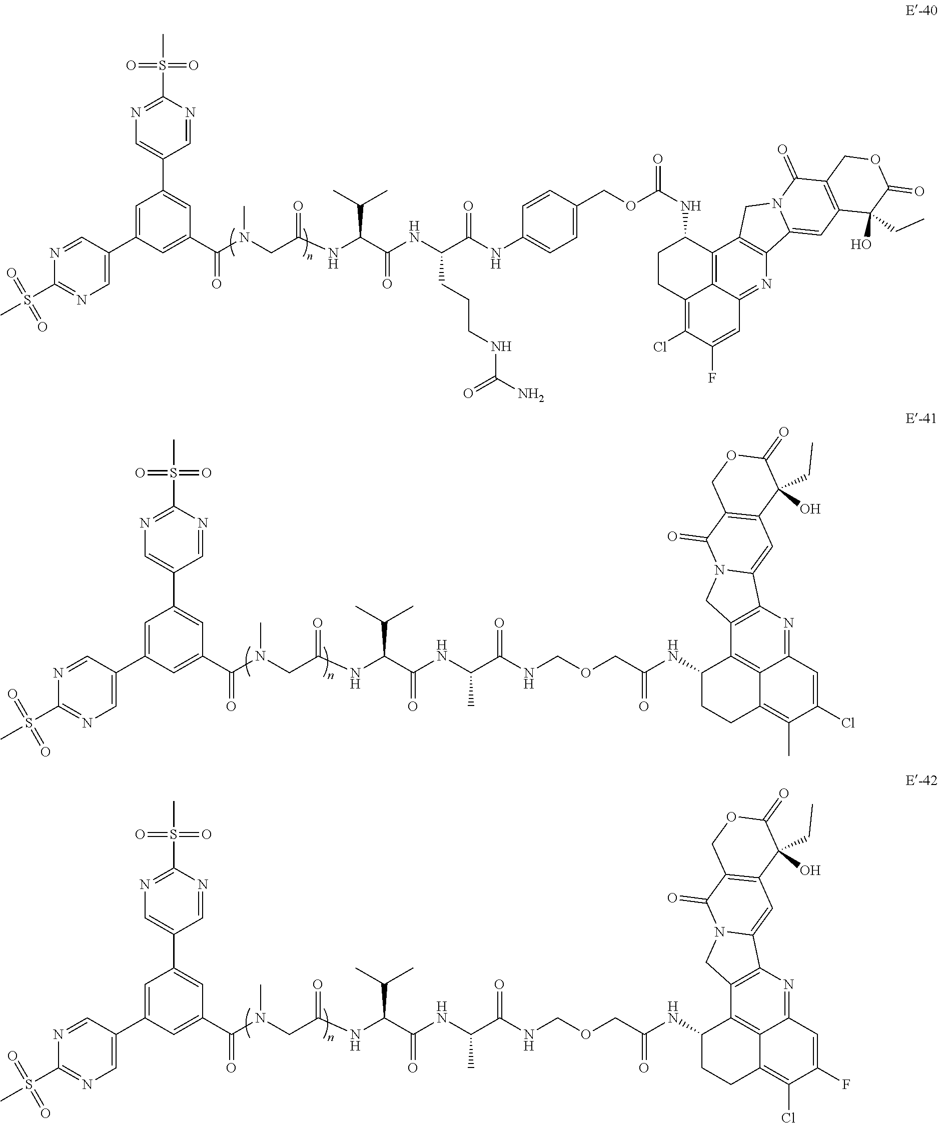

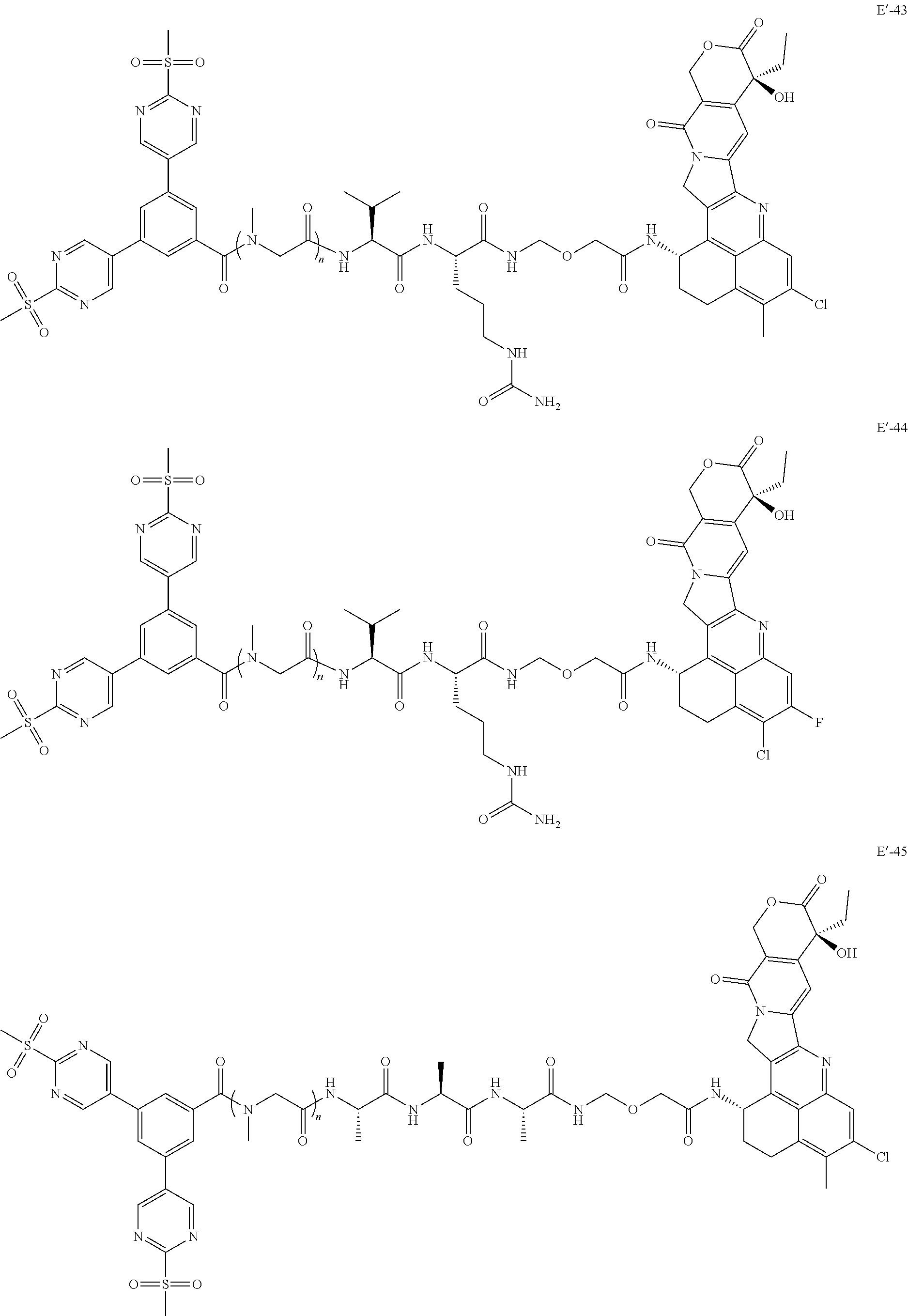

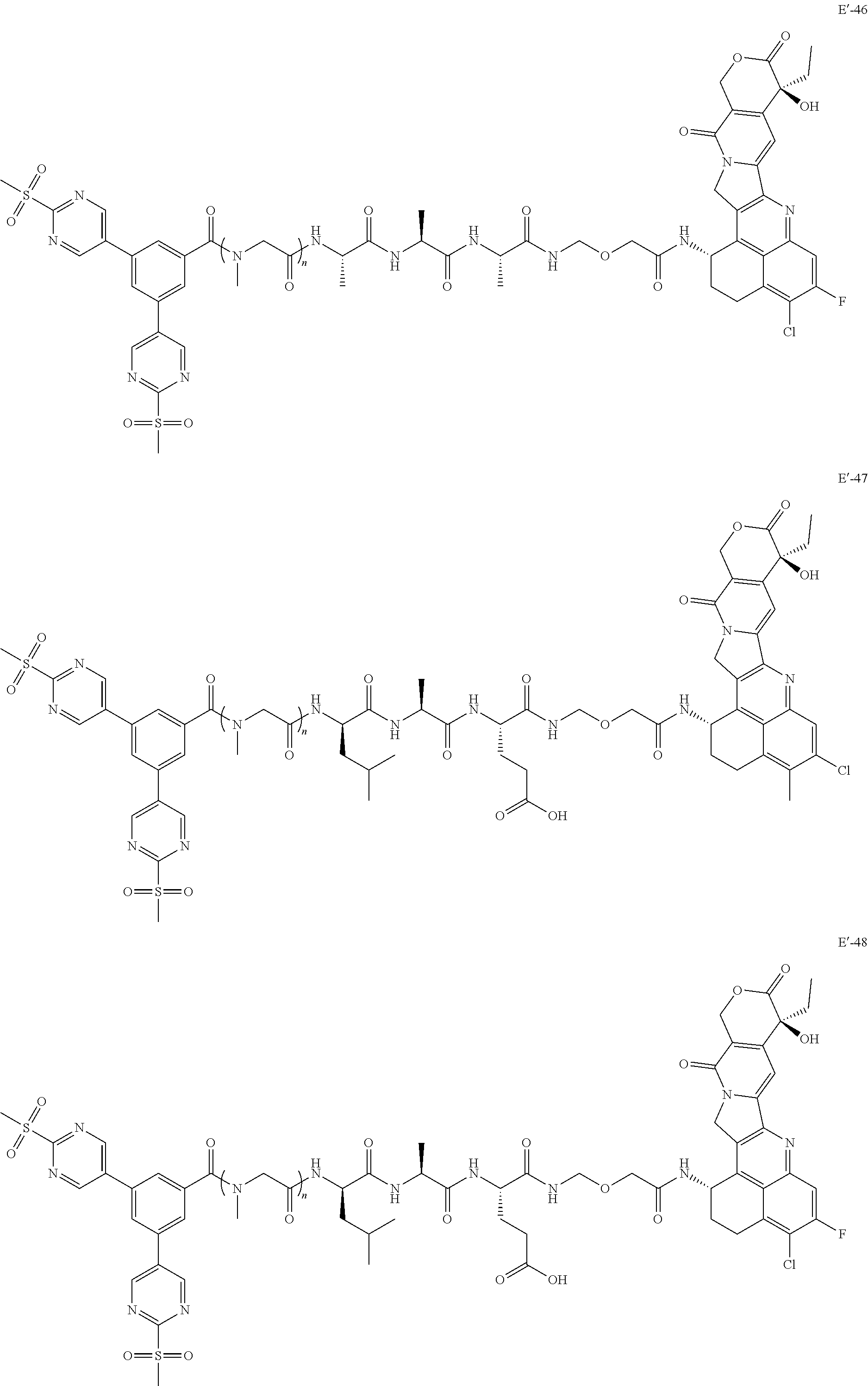

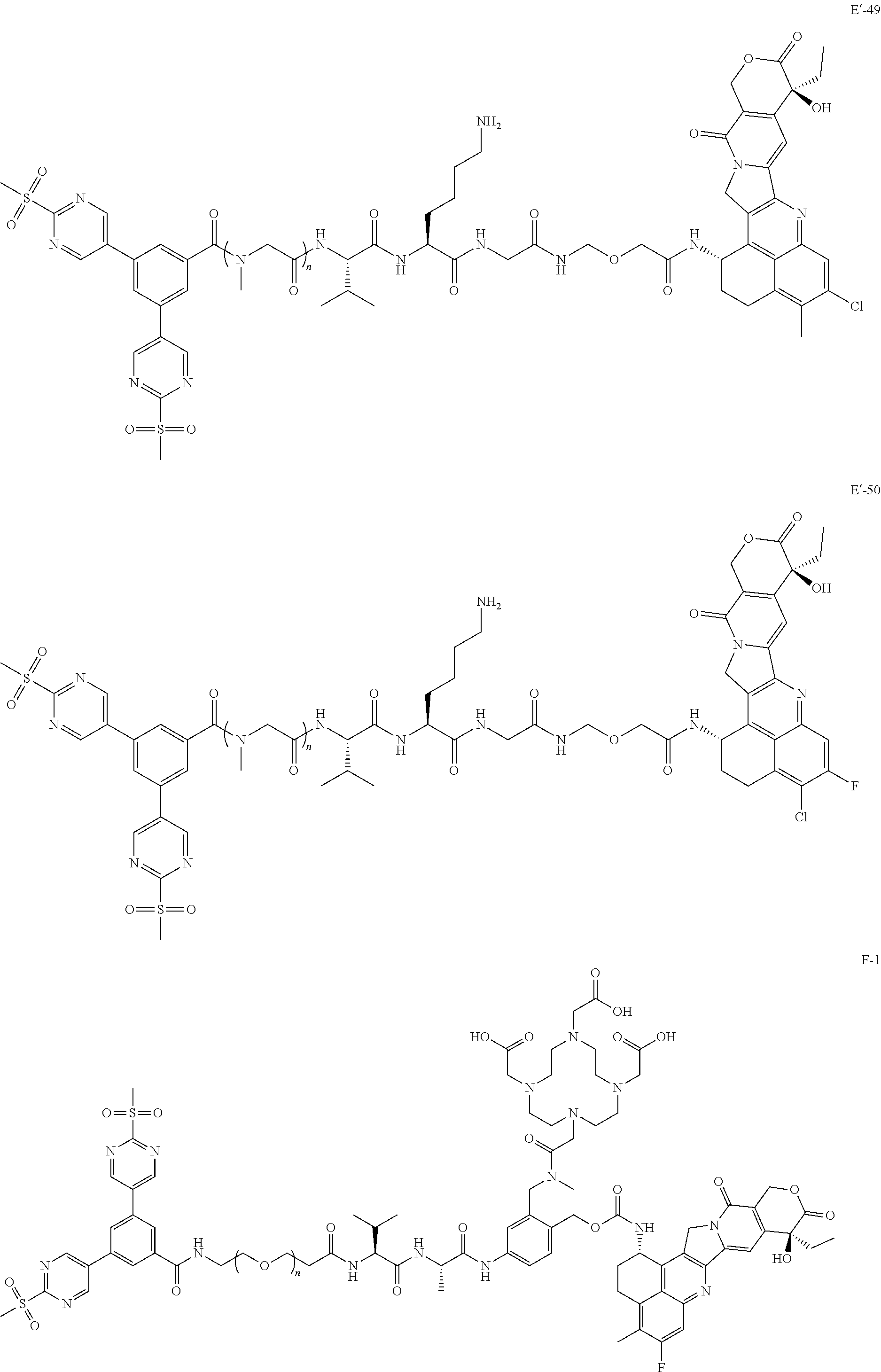

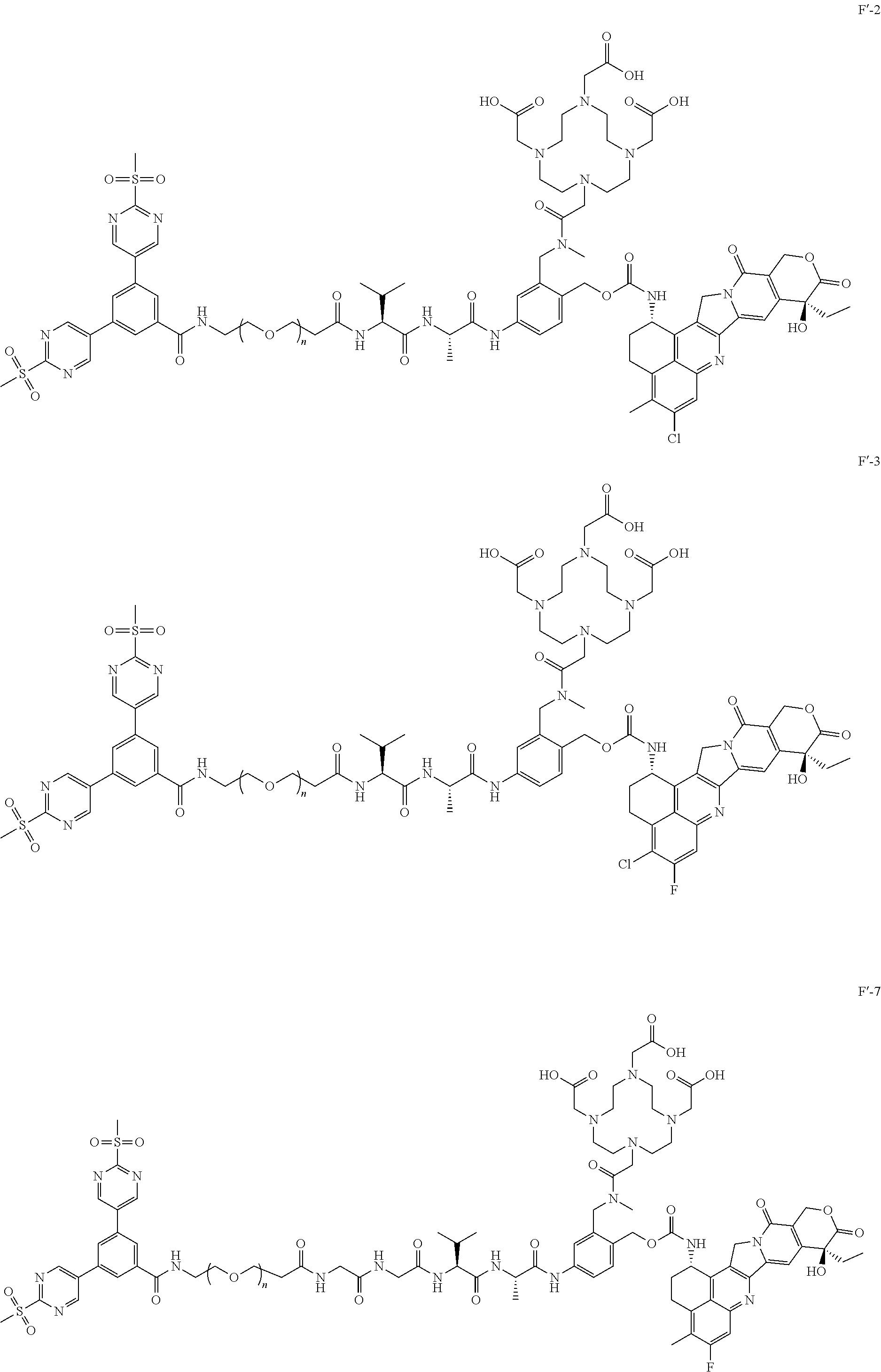

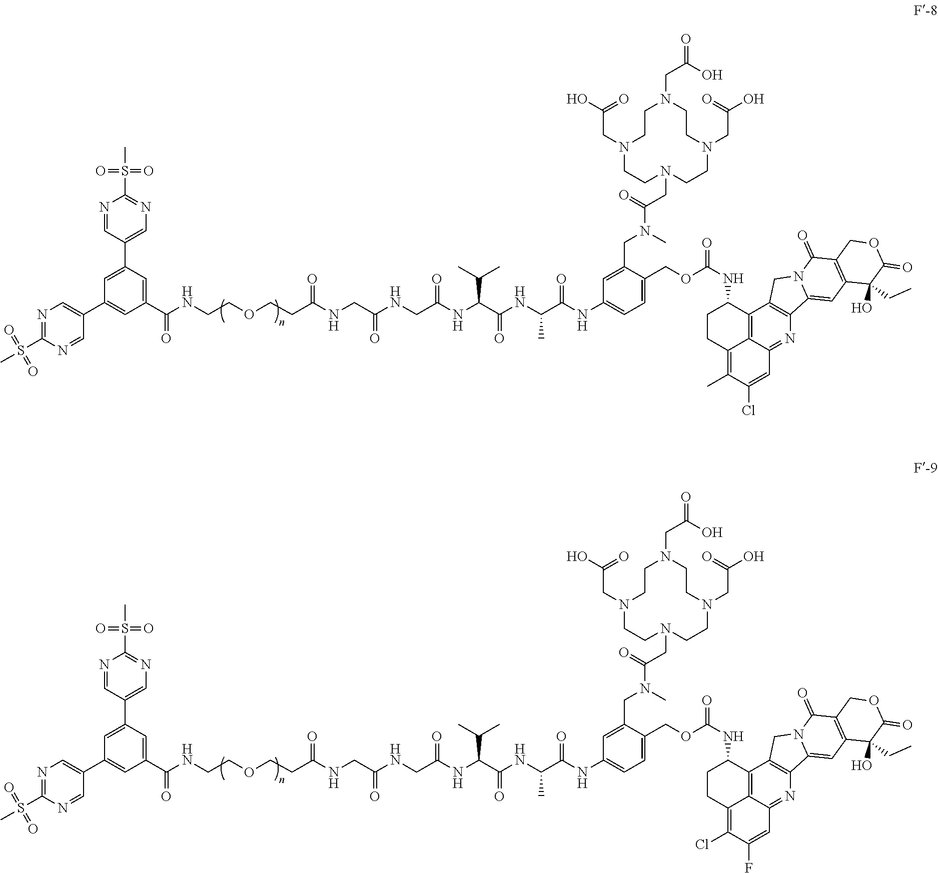

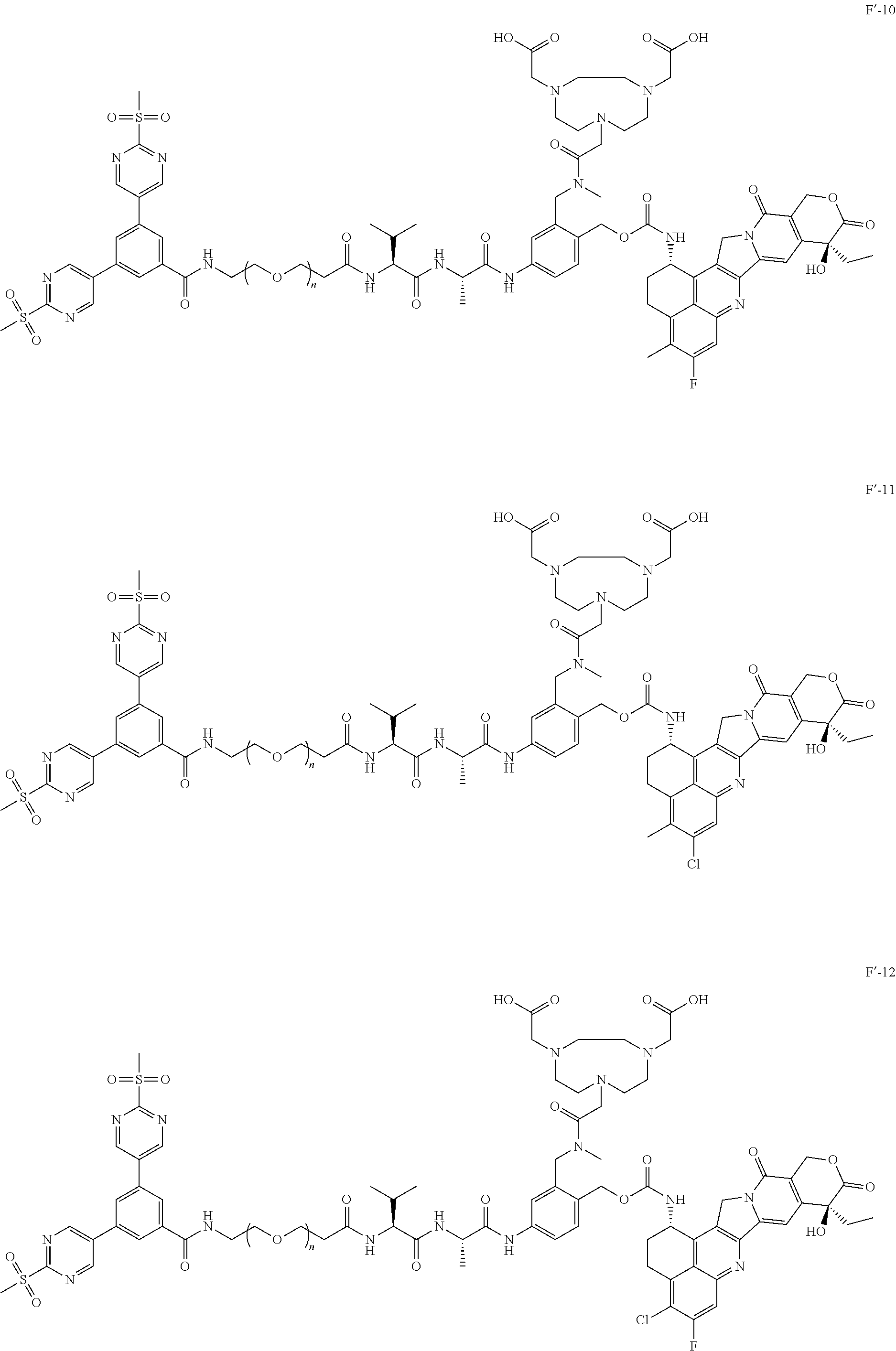

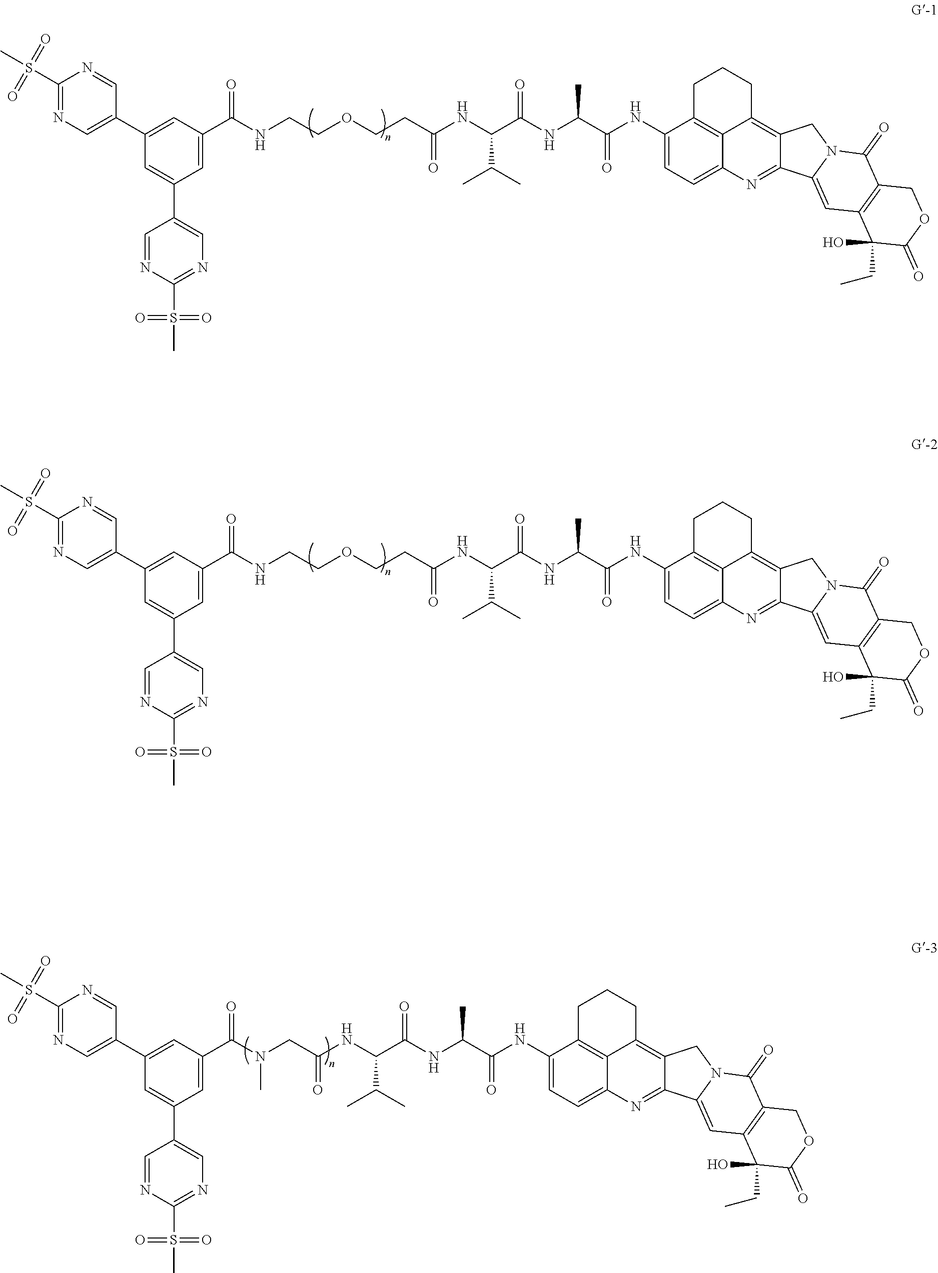

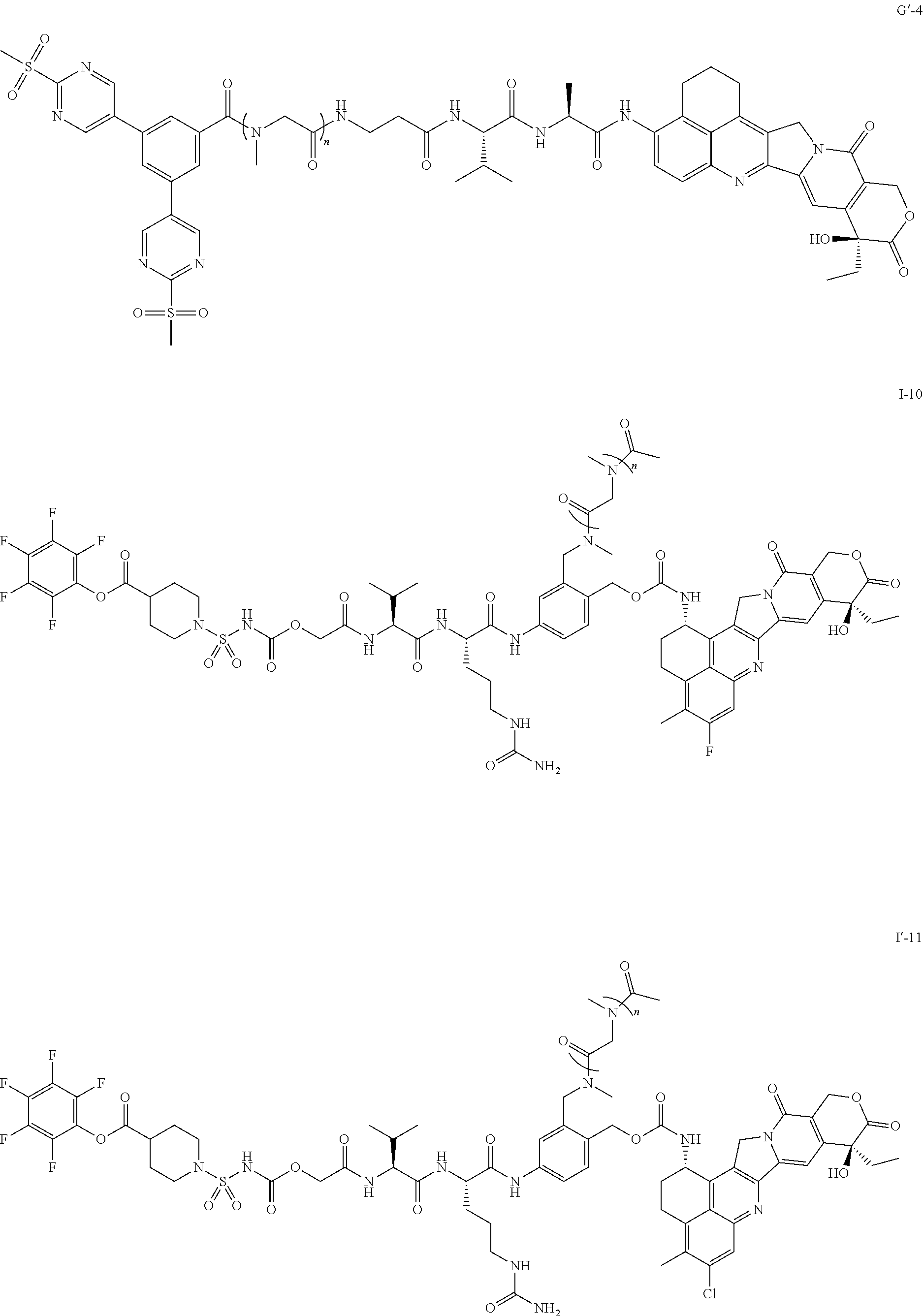

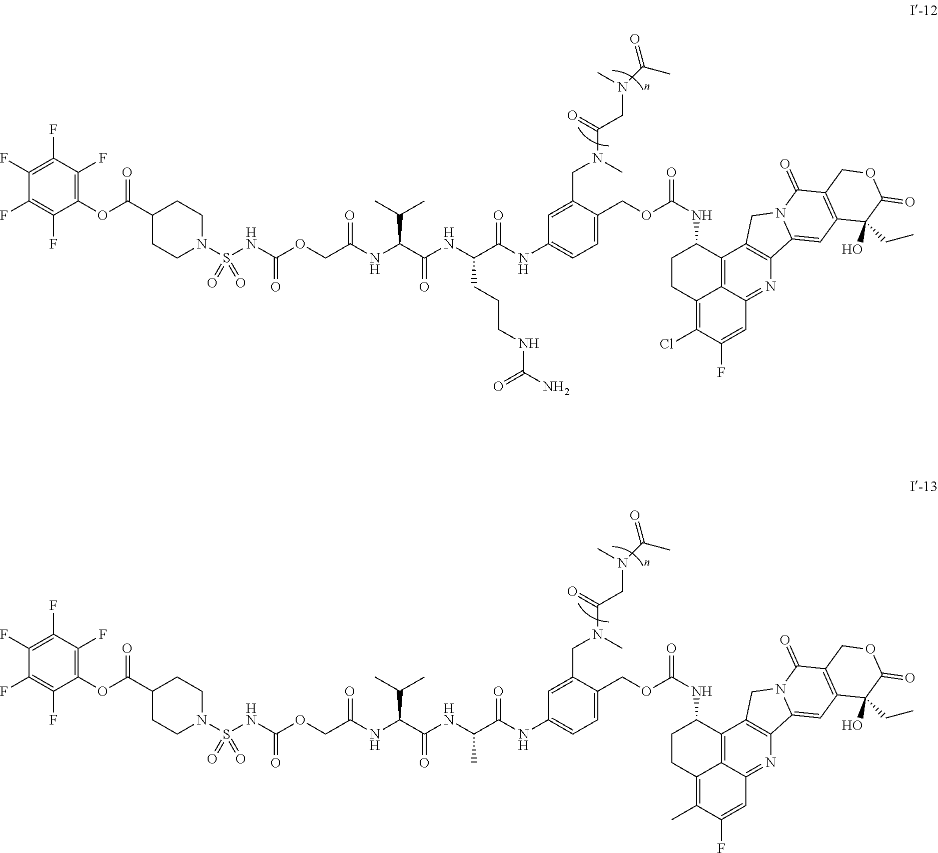

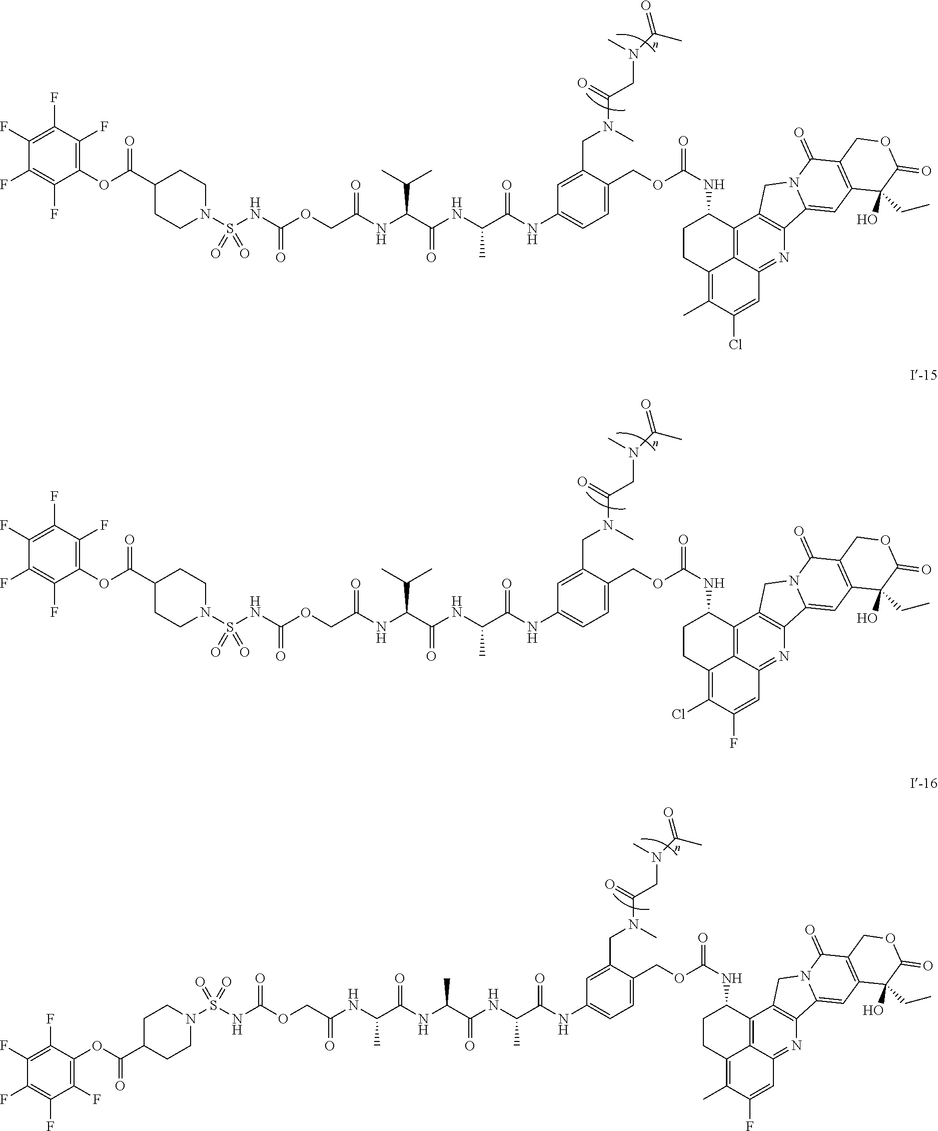

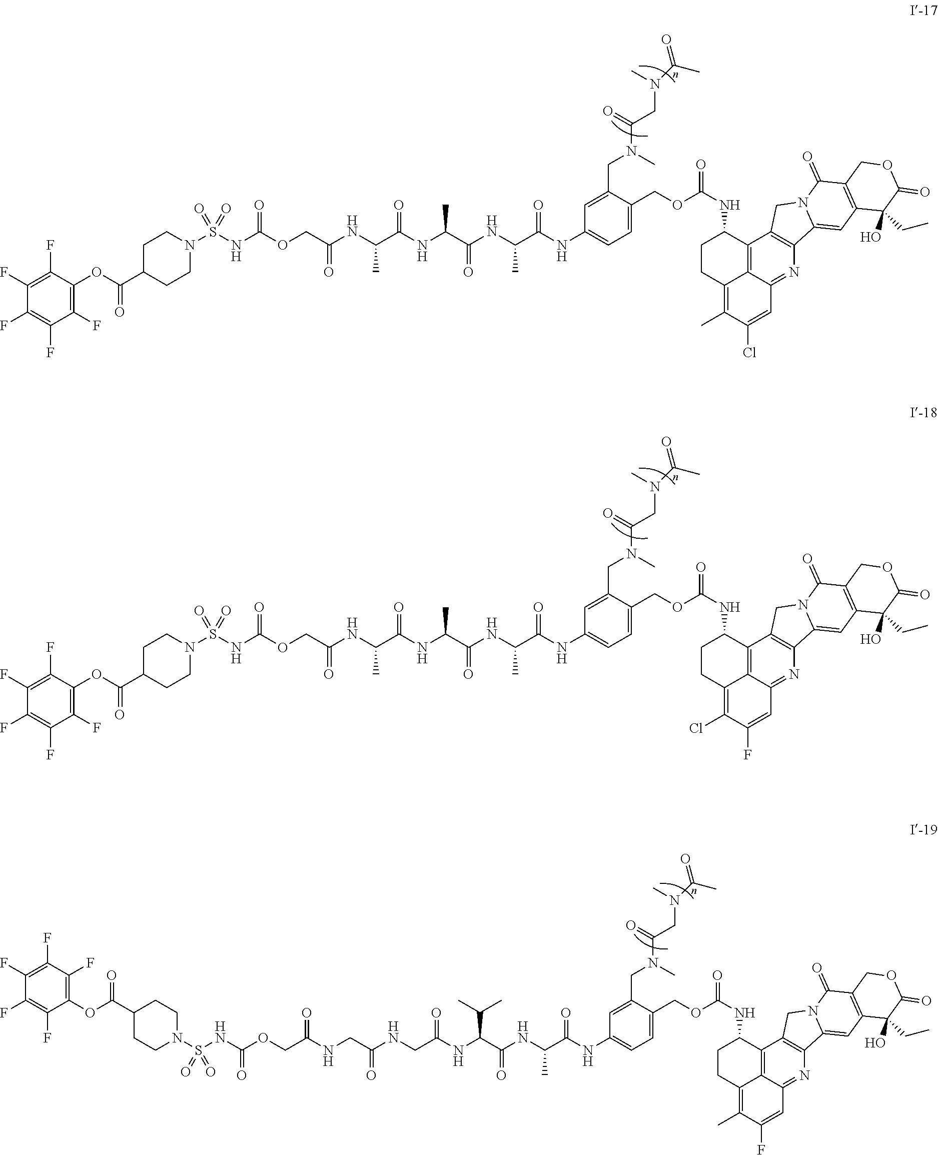

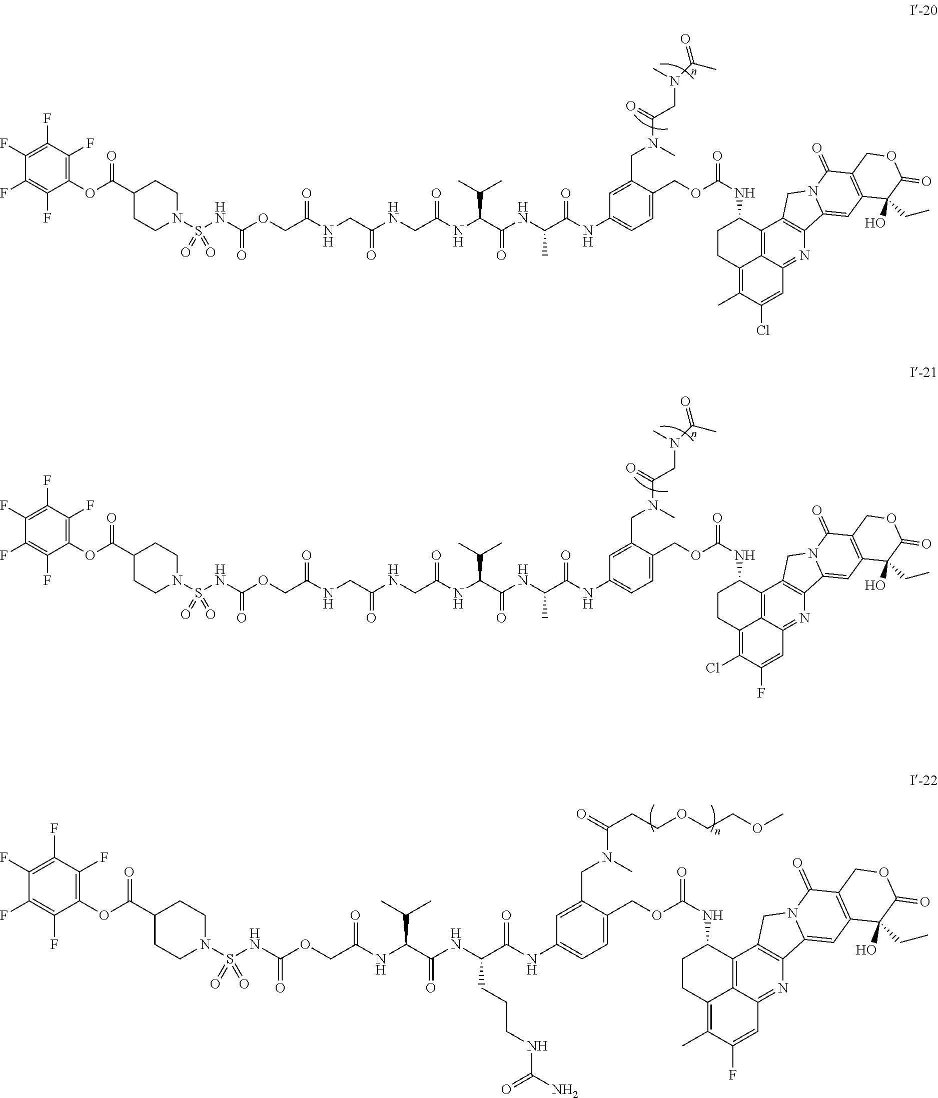

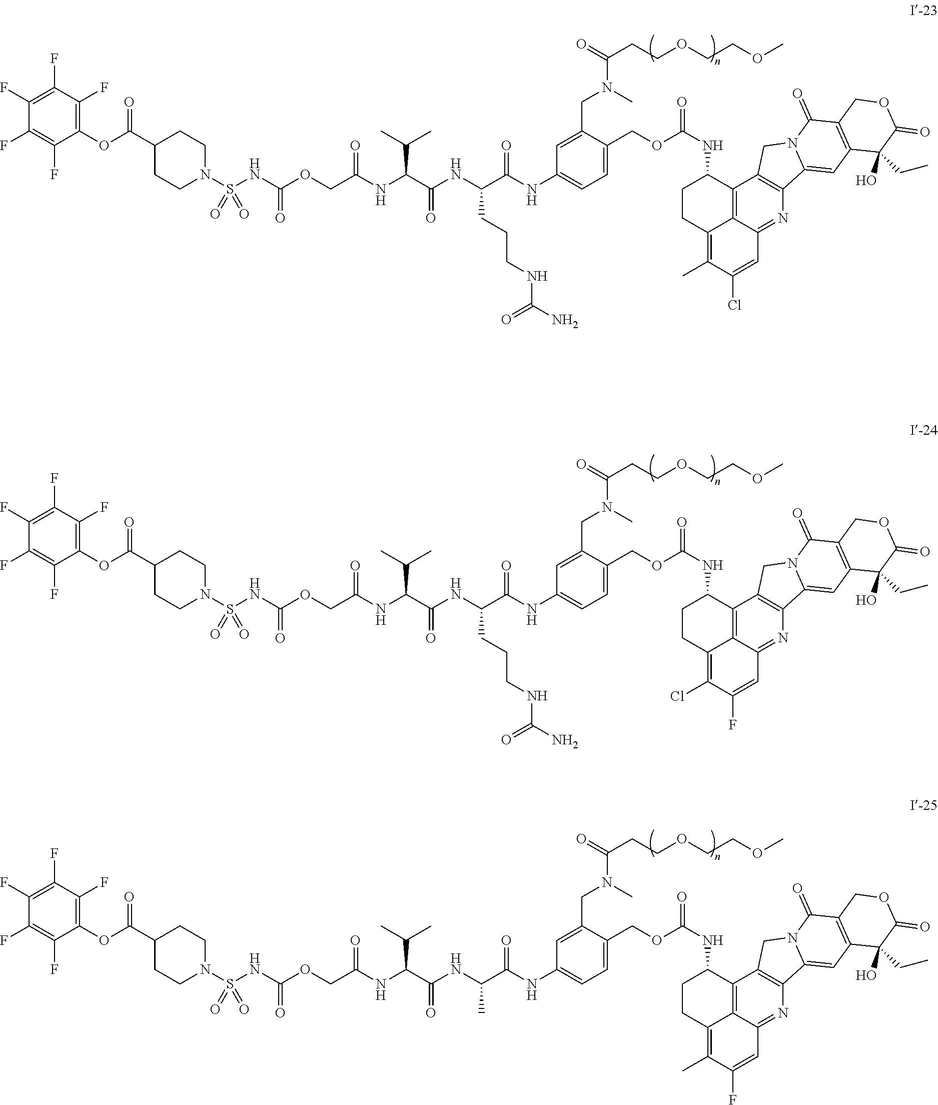

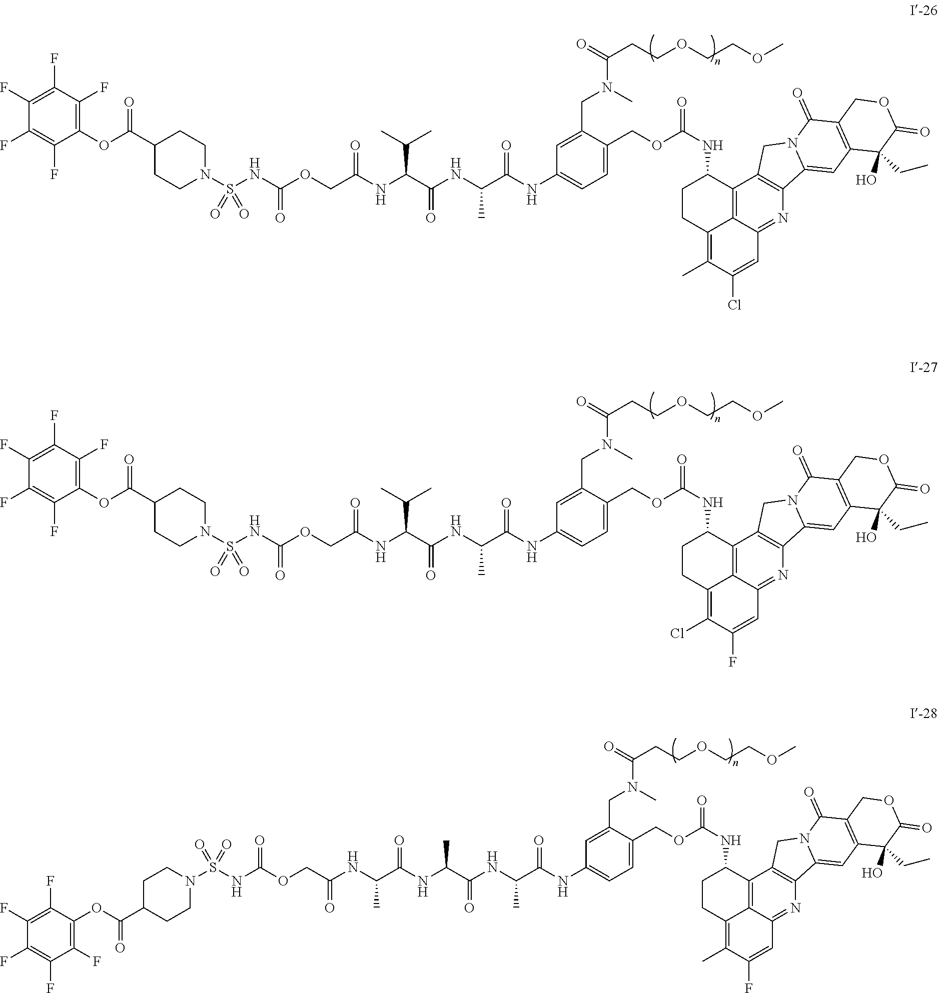

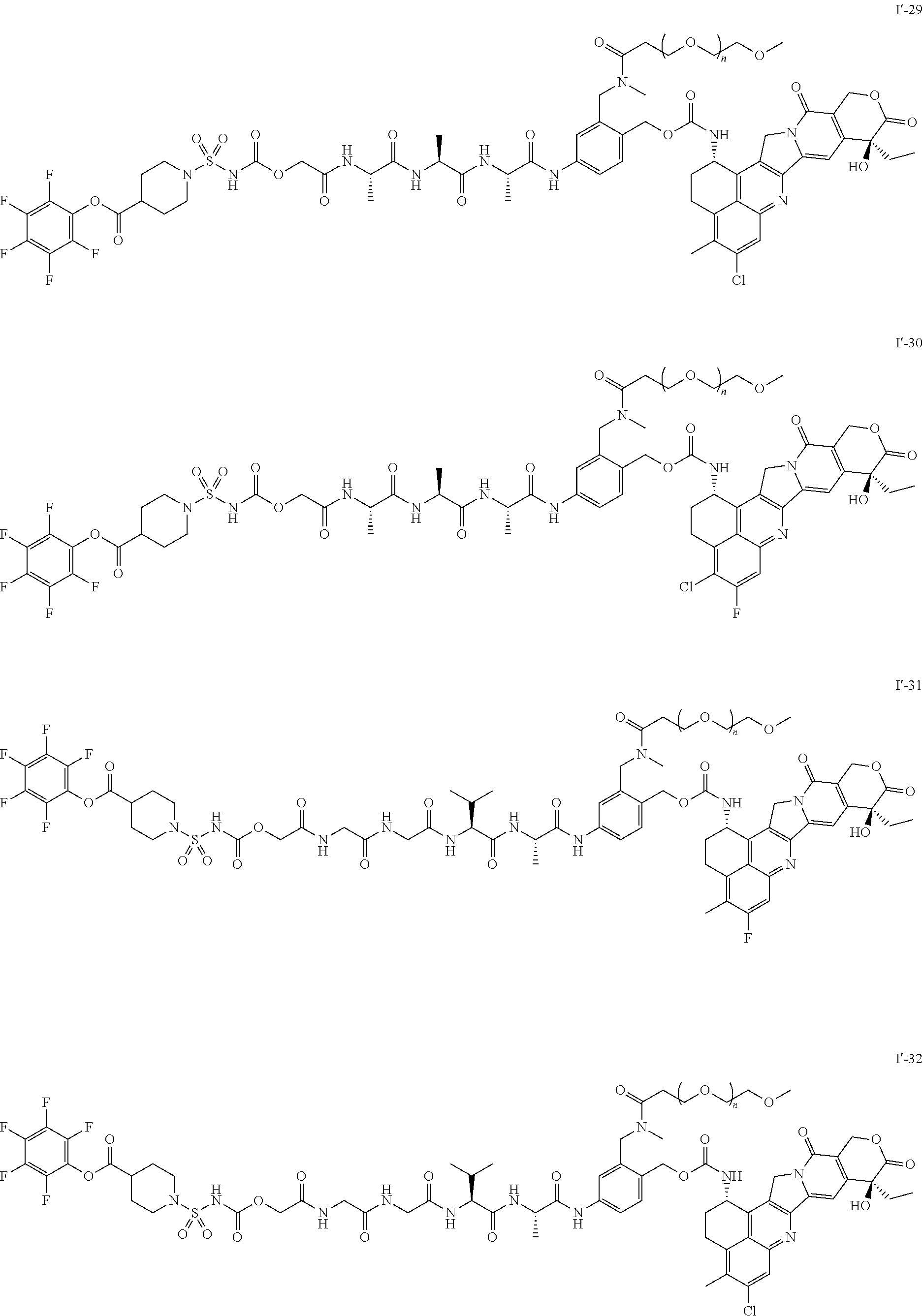

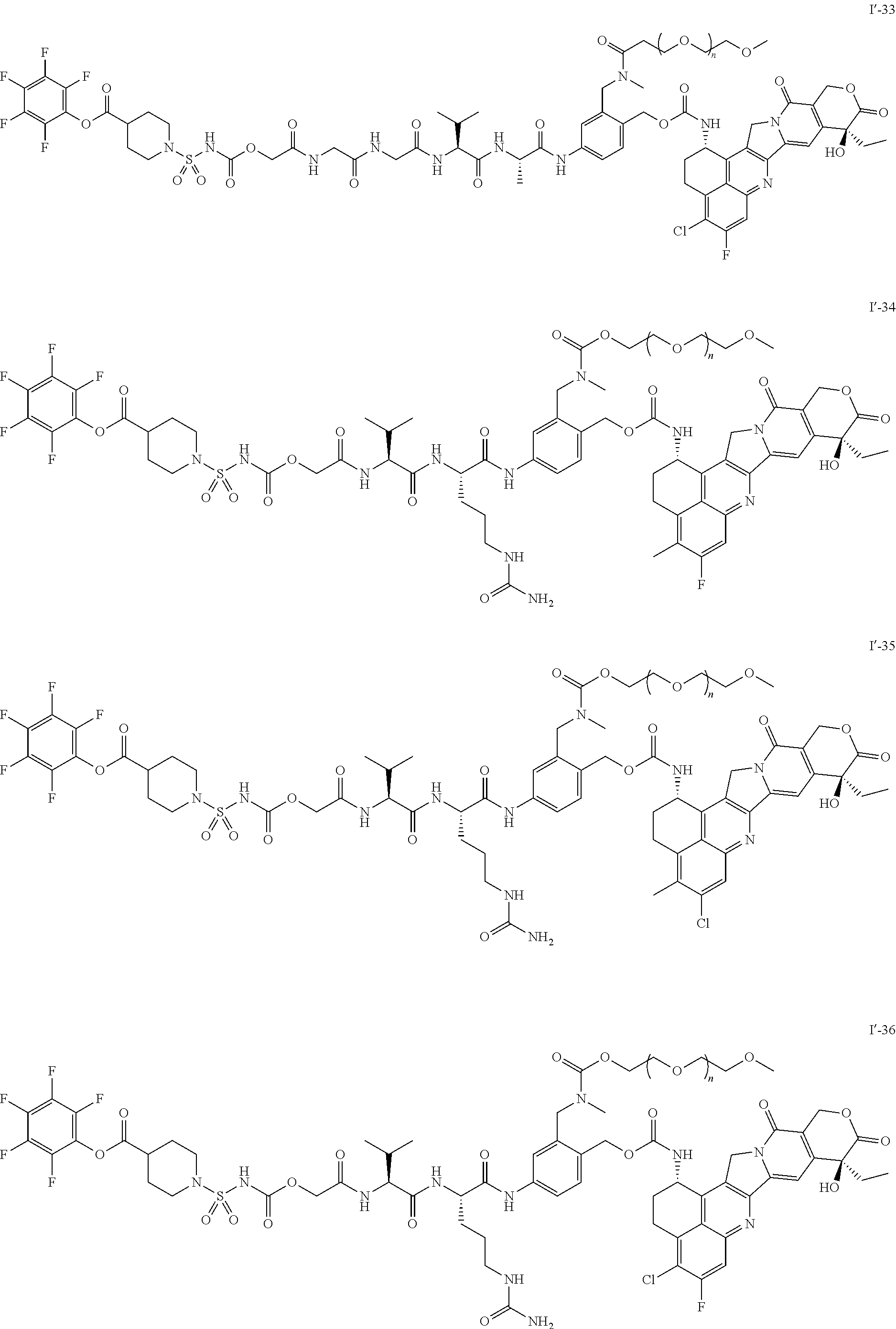

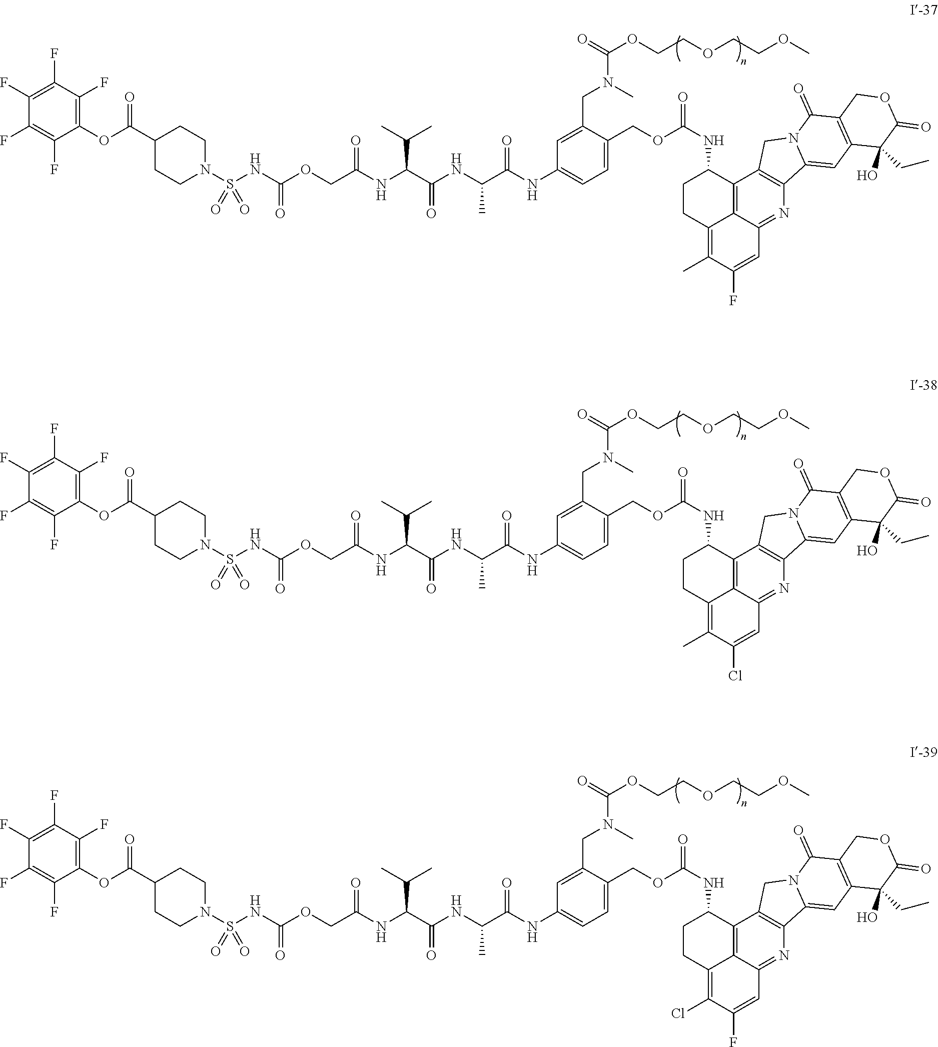

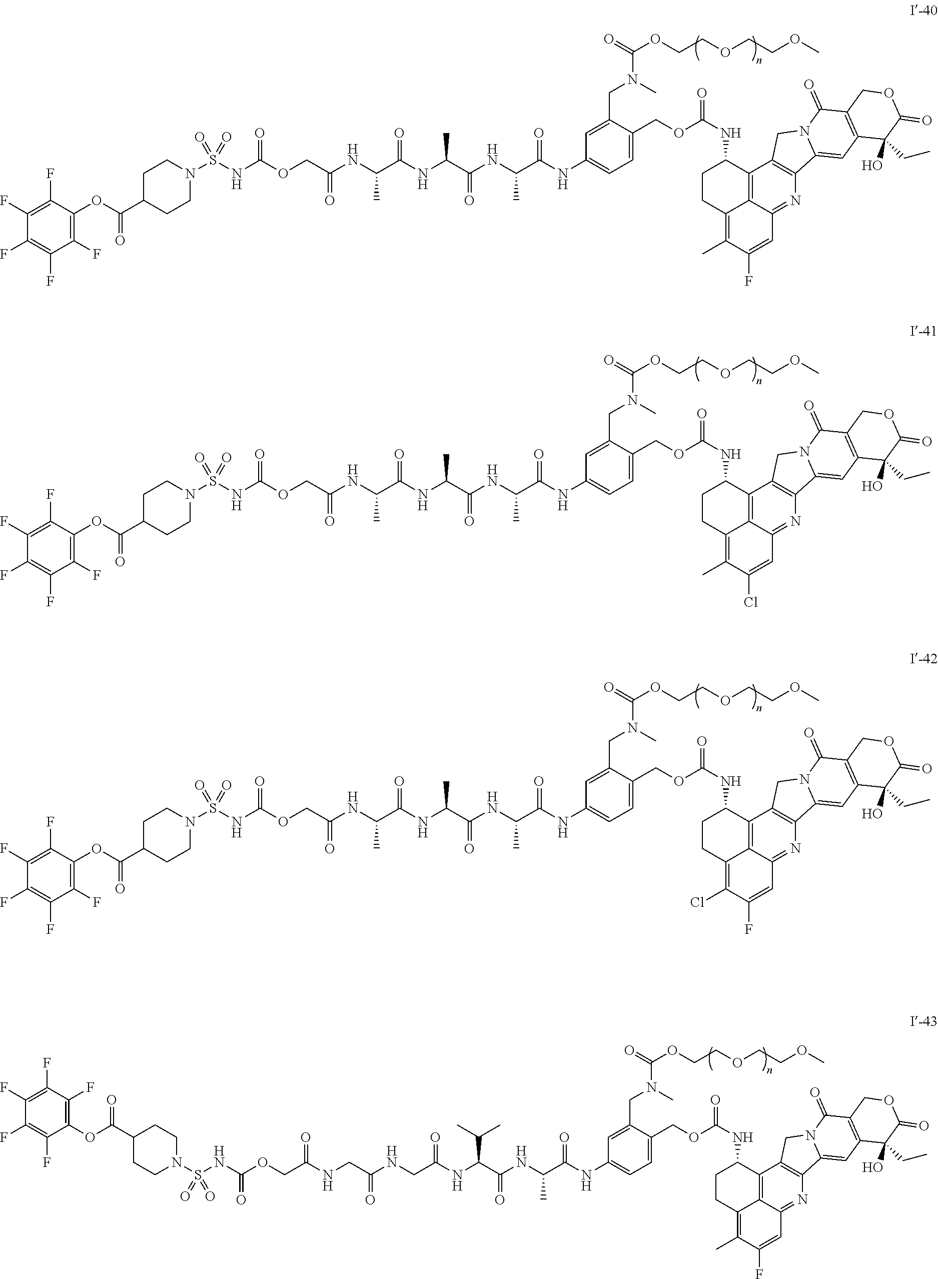

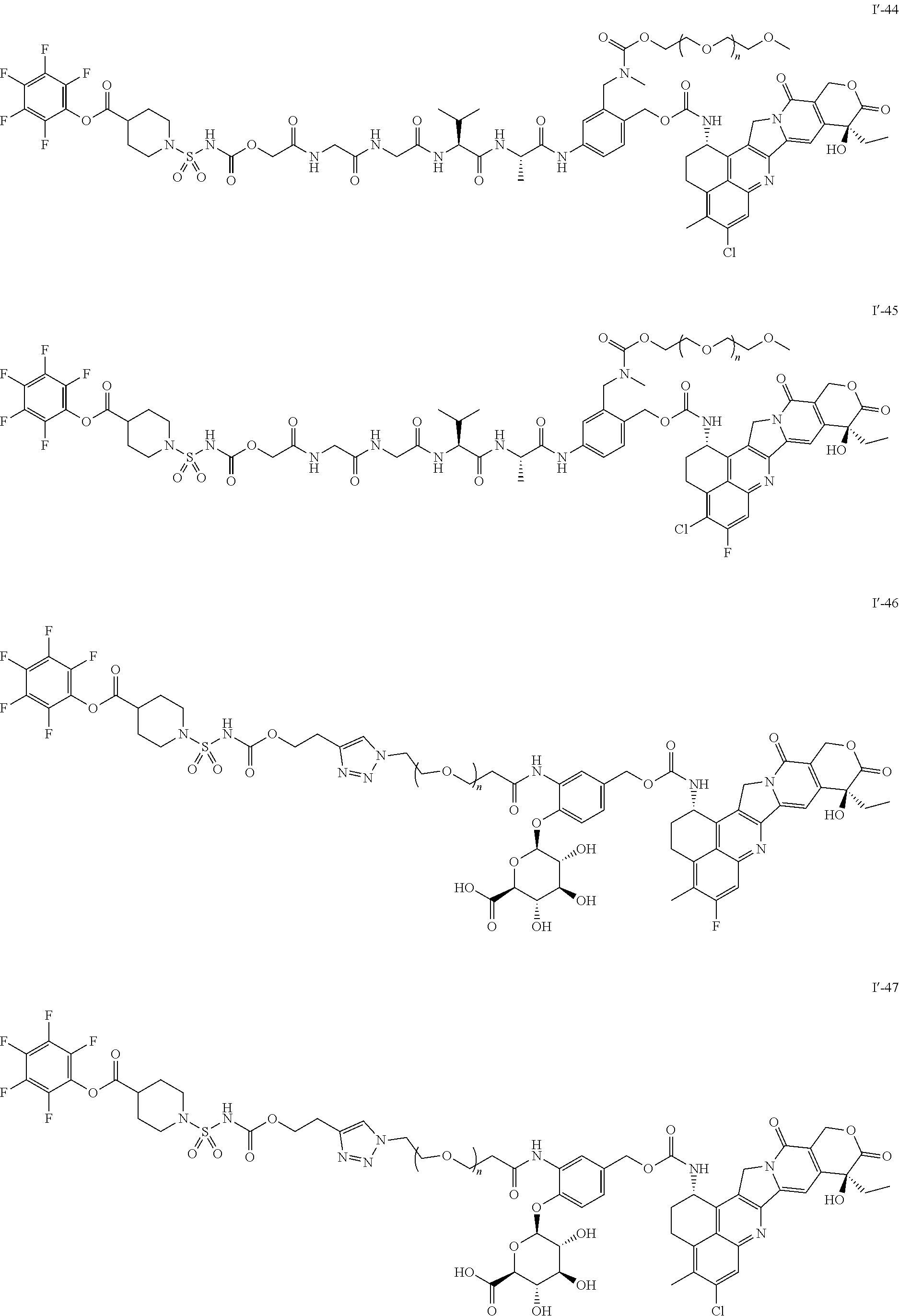

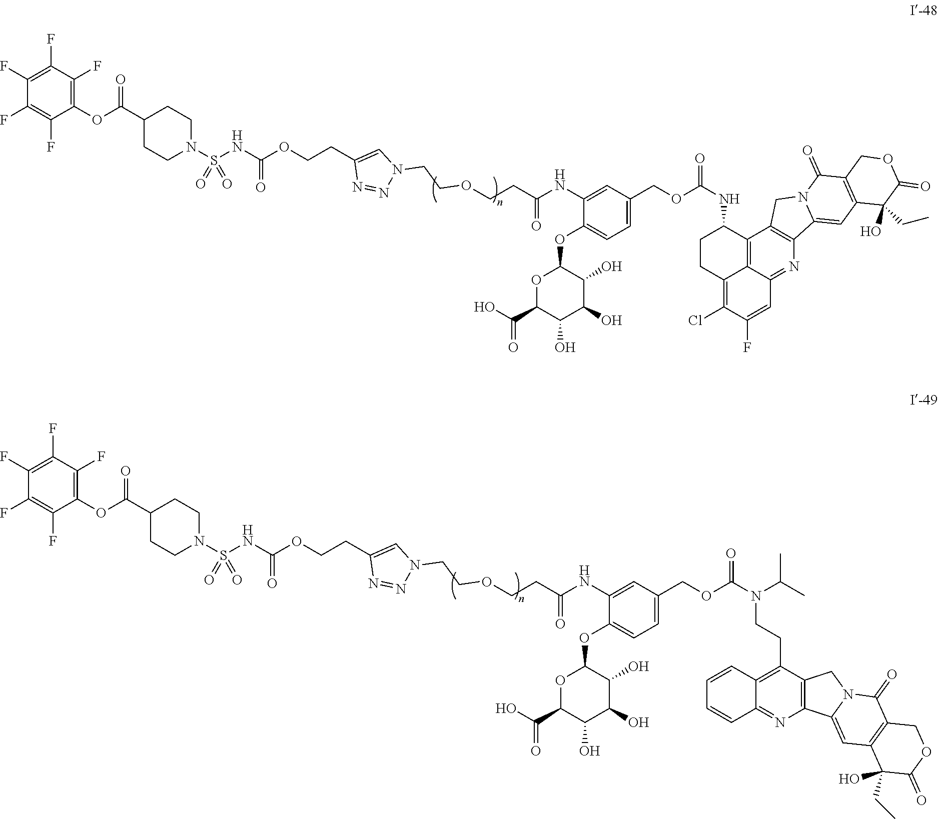

wherein n is an integer selected from 1 to 20, such as an integer of 1 to 12, 3 to 12, for example, n is 1, 2, 3, 4, 5, 6, 7, 8, 9, 10, 11, 12, 13, 14 or 15.

In some embodiments, the provided is a compound selected from the group consisting of E′-1 to E′-50, F′-1 to F′-3, F′-7 to F′-12, G′-1 to G′-4, I′-10 to I′-49, E-1 to E-50, F-1 to F-14, G-1 to G-26, I-1 to I-50 or pharmaceutically acceptable salts thereof, wherein n is an integer selected from 1 to 20, such as an integer of 1 to 12, 3 to 12, for example, n is 1, 2, 3, 4, 5, 6, 7, 8, 9, 10, 11, 12, 13, 14 or 15.

In some embodiments, the provided is a compound selected from the group consisting of E-1 to E-50, F-1 to F-14, G-1 to G-24 or pharmaceutically acceptable salts thereof, wherein n is an integer selected from 1 to 20, such as an integer of 1 to 12, 3 to 12, for example, n is 1, 2, 3, 4, 5, 6, 7, 8, 9, 10, 11, 12, 13, 14 or 15.

In some embodiments, the provided is a compound selected from the group consisting of E-1 to E-50, F-1 to F-12 or pharmaceutically acceptable salts thereof.

In some embodiments, the provided is a compound selected from the group consisting of E-1 to E-50, I-1 to I-49 or pharmaceutically acceptable salts thereof.

In some embodiments, the compound or pharmaceutically acceptable salt thereof described above may be optionally substituted with one or more suitable substituents.

In some embodiments, the antibody-drug conjugate of the present application has a structure represented by the formula Ab-[M-L-E-D]ₓ, wherein:
Ab represents an antibody or antigen-binding fragment capable of specifically binding to epidermal growth factor receptor 2 (Her2), a member of the ErbB family of receptor tyrosine kinases;
x is 1 to 10, preferably, x is 1 to 4, and further preferably, x is 2 or 4;
-M-L-E-D is formed from the compounds E-1 to E-50, F-1 to F-14, G-1 to G-26, or I-1 to I-50 as described above.

In some embodiments, the antibody-drug conjugate of the present application has a structure represented by the formula Ab-[M-L-E-D]ₓ, wherein:
Ab represents an antibody or antigen-binding fragment thereof capable of specifically binding to epidermal growth factor receptor 2 (Her2), a member of the ErbB family of receptor tyrosine kinases;
x is 1 to 10, preferably, x is 1 to 4, and more preferably, x is 4;
-M-L-E-D is formed by removing the methylsulfonyl group from the aforementioned compounds E-1 to E-50, F-1 to F-14 or G-1 to G-26.

In some embodiments, the antibody-drug conjugate of the present application has a structure represented by the formula Ab-[M-L-E-D]ₓ, wherein:
Ab represents an antibody or antigen-binding fragment thereof capable of specifically binding to epidermal growth factor receptor 2 (Her2), a member of the ErbB family of receptor tyrosine kinases;
x is 1 to 10, preferably, x is 1 to 4, and further preferably, x is 2;
-M-L-E-D is formed by removing the pentafluorophenoxy group from the compounds I-1 to I-50 as described above.

In some embodiments, the antibody or antigen-binding fragment thereof comprises:
(1) the following heavy chain variable region (VH) and/or light chain variable region (VL):
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL):
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL):
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a) and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL):
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following three CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof, or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some embodiments, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof, and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof, and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a) and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable regions (VH) and/or light chain variable regions (VL), wherein the CDRs are defined according to the IMGT numbering system:
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof, and/or, a light chain variable region (VL) comprising the following three CDRs: a CDR-L1 having a sequence as set forth in SEQ ID NO: 16 or variant thereof, and a CDR-L2 having a sequence as set forth in SEQ ID NO: 17 or variant thereof, and a CDR-L3 having a sequence as set forth in SEQ ID NO: 10 or variant thereof, or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof,
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some embodiments, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and/or light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and/or, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and/or, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25.

In some embodiments, the antibody or antigen-binding fragment thereof comprises:
(1) the following heavy chain variable region (VH) and light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25.

In some embodiments, the antibody or antigen-binding fragment thereof comprises:
(a) a VH as set forth in SEQ ID NO: 1 or variant thereof, and/or, a VL as set forth in SEQ ID NO: 2 or variant thereof; or
(b) a VH as set forth in SEQ ID NO: 3 or variant thereof, and/or, a VL as set forth in SEQ ID NO: 4 or variant thereof;
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution.

In some embodiments, the antibody or antigen-binding fragment thereof comprises:
(a) a VH as set forth in SEQ ID NO: 1, and/or, a VL as set forth in SEQ ID NO: 2; or
(b) a VH as set forth in SEQ ID NO: 3, and/or, a VL as set forth in SEQ ID NO: 4.

In some embodiments, the antibody or antigen-binding fragment thereof comprises:
(a) a VH as set forth in SEQ ID NO: 1 or variant thereof, and, a VL as set forth in SEQ ID NO: 2 or variant thereof; or
(b) a VH as set forth in SEQ ID NO: 3 or variant thereof, and, a VL as set forth in SEQ ID NO: 4 or variant thereof;
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution.

In some embodiments, the antibody or antigen-binding fragment thereof comprises:
(a) a VH as set forth in SEQ ID NO: 1, and a VL as set forth in SEQ ID NO: 2; or
(b) a VH as set forth in SEQ ID NO: 3, and a VL as set forth in SEQ ID NO: 4.

In some embodiments, the antibody or antigen-binding fragment thereof further comprises:
(a) a heavy chain constant region (CH) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids); and
(b) a light chain constant region (CL) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids).

In some embodiments, the heavy chain constant region is an IgG heavy chain constant region, such as an IgG1, IgG2, IgG3 or IgG4 heavy chain constant region, such as a human IgG1 heavy chain constant region or a human IgG4 heavy chain constant region.

In some embodiments, the antibody or antigen-binding fragment thereof comprises a heavy chain constant region (CH) as set forth in SEQ ID NO: 35 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 35.

In some embodiments, the antibody or antigen-binding fragment thereof comprises a heavy chain constant region (CH) as set forth in SEQ ID NO: 35.

In some embodiments, the antibody or antigen-binding fragment thereof comprises a light chain constant region (CL) as set forth in SEQ ID NO: 36 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 36.

In some embodiments, the antibody or antigen-binding fragment thereof comprises a light chain constant region (CL) as set forth in SEQ ID NO: 36.

In some embodiments, the antibody or antigen-binding fragment thereof comprises a heavy chain constant region (CH) as set forth in SEQ ID NO: 35 and a light chain constant region (CL) as set forth in SEQ ID NO: 36.

In some embodiments, the antibody or antigen-binding fragment thereof comprises:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36.

In some embodiments, the antibody or antigen-binding fragment thereof comprises:
(1) a heavy chain comprising the sequence as set forth in SEQ ID NO: 37, and, a light chain comprising the sequence as set forth in SEQ ID NO: 38; or
(2) a heavy chain comprising the sequence as set forth in SEQ ID NO: 39, and, a light chain comprising the sequence as set forth in SEQ ID NO: 40.

In certain embodiments, the antibody or antigen-binding fragment disclosed herein has a heavy chain constant region that may comprise a C-terminal lysine lack a C-terminal lysine or a C-terminal glycine-lysine dipeptide. In some embodiments, the N-terminal amino acid of the antibody or antigen-binding fragment thereof may be cyclized to pyroglutamic acid.

As known to those skilled in the art, pyroglutamic acid is the conjugate acid of pyroglutamate and is in equilibrium with pyroglutamate in solution.

In certain embodiments, provided herein is a composition comprising the antibody or antigen-binding fragments disclosed herein, wherein each antibody or antigen-binding fragment thereof may independently comprise a C-terminal lysine, lack a C-terminal lysine, lack a C-terminal glycine-lysine and/or comprise a N-terminal glutamine or glutamic acid, or a N-terminal amino acid cyclized to pyroglutamic acid or pyroglutamate.

In certain embodiments, the antibody or antigen-binding fragment disclosed herein comprises those capable of specifically binding to an antigen and may comprise post-translational modifications thereof (e.g., cleavage of the C-terminal lysine in the heavy chain, conversion of the N-terminal glutamine or glutamic acid in the heavy or light chain to pyroglutamic acid or pyroglutamate), which may occur during recombinant expression in a host cell (e.g., CHO cell) or arise during purification/storage.

In certain embodiments, the N-terminal glutamine of the VH having the sequence as set forth in SEQ ID NO: 1 or 3 or variant thereof or the heavy chain having the sequence as set forth in SEQ ID NO: 37 or 39 or variant thereof undergoes cyclization to form pyroglutamic acid or pyroglutamate.

In certain embodiments, the heavy chain constant region (CH) as set forth in SEQ ID NO: 35 or variant thereof or the heavy chain having the sequence as set forth in SEQ ID NO: 37 or 39 or variant thereof lacks C-terminal lysine.

In some embodiments, the antibody or antigen-binding fragment thereof is selected from Trastuzumab or Pertuzumab. The query accession number (IMGT/mAb-DB ID) for the amino acid sequence of Trastuzumab in the IMGT database is: 97. The query accession number (IMGT/mAb-DB ID) for the amino acid sequence of Pertuzumab in the IMGT database is: 80.

In some embodiments, M is connected to the sulfydryl group (—SH) or amino group (—NH₂) on Ab.

In some embodiments, M is connected to the sulfydryl group (—SH) on Ab.

In some embodiments, in the antibody-drug conjugate having a structure represented by Ab-[M-L-E-D]ₓ, M-L-E-D is formed from a compound represented by D-E-L-M′, preferably by removing Lg from D-E-L-M′, wherein the compound represented by D-E-L-M′ is as defined above, Ab is as defined above, and x is 1 to 10.

In some embodiments, in the antibody-drug conjugate having a structure represented by Ab-[M-L-E-D]ₓ, M-L-E-D is formed from a compound as shown in any one of E′-1 to E′-50, F′-1 to F′-3, F′-7 to F′-12, G′-1 to G′-4, I′-10 to I′-49, E-1 to E-50, F-1 to F-14, G-1 to G-26, I-1 to I-50, preferably formed by removing —SO₂Me or pentafluorophenoxy from the compound, Ab is as defined above, and x is 1 to 10.

In certain embodiments, the antibody in the antibody-drug conjugate is conjugated to 1 to 4 (e.g., 1, 2, 3 or 4) of the following structure:

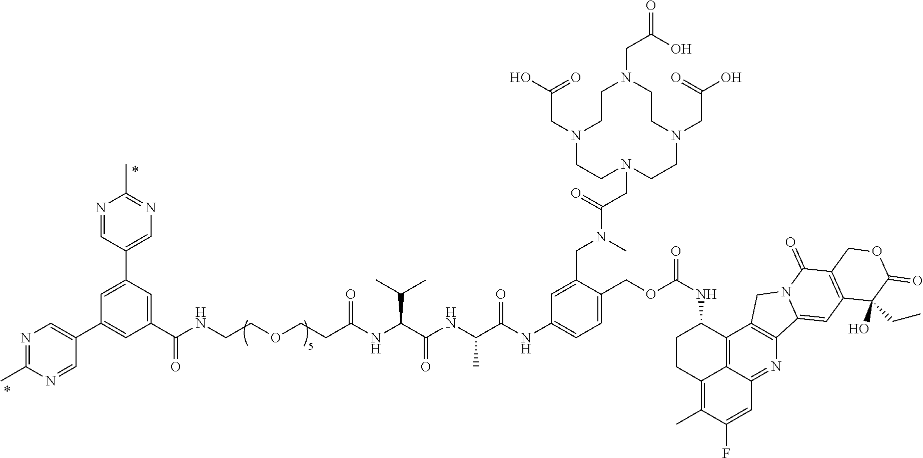

wherein * is the connection site of the sulfhydryl group of the cysteine of the conjugated antibody.

In certain embodiments, the antibody in the antibody-drug conjugate is conjugated to 1 to 4 (e.g., 1, 2, 3, or 4) of the following structures:

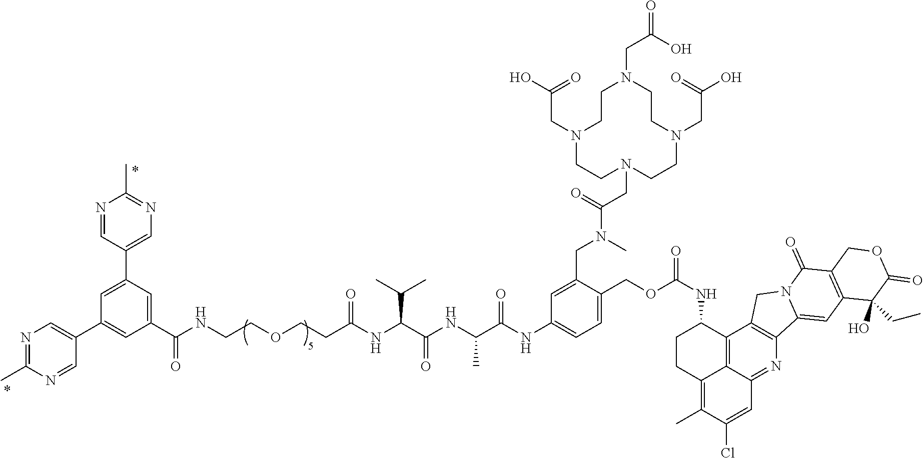

wherein * is the connection site of the sulfhydryl group of the cysteine of the conjugated antibody.

In certain embodiments, the antibody in the antibody-drug conjugate is conjugated to 1 to 4 (e.g., 1, 2, 3, or 4) of the following structures:

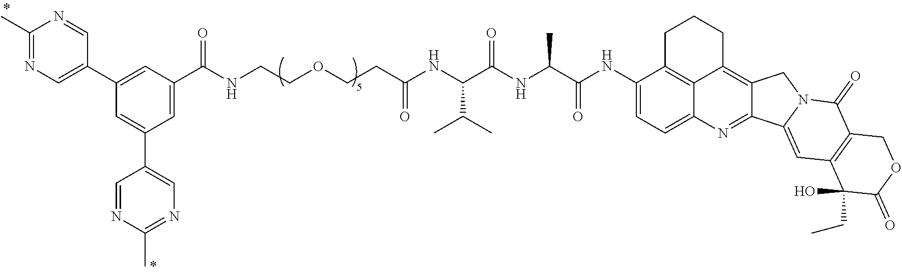

wherein * is the connection site of the sulfhydryl group of the cysteine of the conjugated antibody.

In certain embodiments, the antibody in the antibody-drug conjugate is conjugated to 1 to 4 (e.g., 1, 2, 3, or 4) of the following structures:

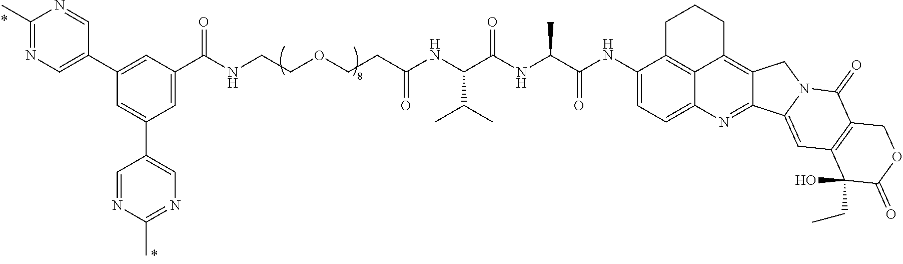

wherein * is the connection site of the sulfhydryl group of the cysteine of the conjugated antibody.

In certain embodiments, the antibody in the antibody-drug conjugate is conjugated to 1 to 4 (e.g., 1, 2, 3, or 4) of the following structures:

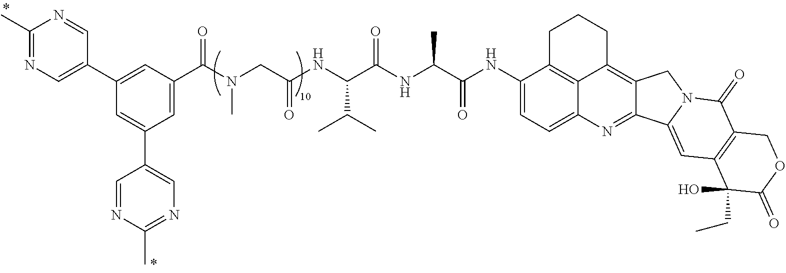

wherein * is the connection site of the sulfhydryl group of the cysteine of the conjugated antibody.

In certain embodiments, the antibody in the antibody-drug conjugate is conjugated to 1 to 4 (e.g., 1, 2, 3, or 4) of the following structures:

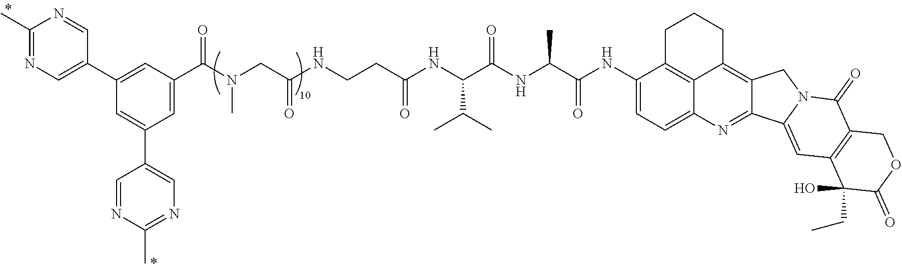

wherein * is the connection site of the sulfhydryl group of the cysteine of the conjugated antibody.

In certain embodiments, the antibody in the antibody-drug conjugate is conjugated to 1 to 4 (e.g., 1, 2, 3, or 4) of the following structures:

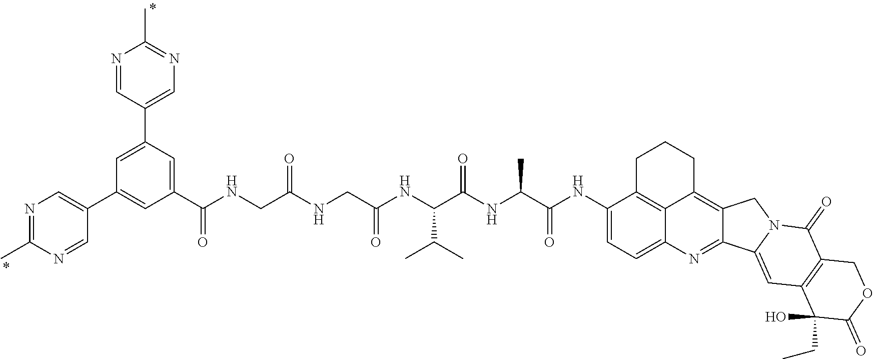 [SMILES: CC[C@@]1(O)C(=O)OCc2c1cc1n(c2=O)Cc2c-1nc1ccc(NC(=O)[C@H](C)NC(=O)[C@@H](NC(=O)CNC(=O)CNC(=O)c3cc(-c4cnc(C)nc4)cc(-c4cnc(C)nc4)c3)C(C)C)c3c1c2CCC3]

wherein * is the connection site of the sulfhydryl group of the cysteine of the conjugated antibody.

In some embodiments, the antibody-drug conjugate is selected from the group consisting of ADC E-1 to ADC E-50, ADC F-1 to ADC F-14, ADC G-1 to G-26, ADC I-1 to I-50 as shown below:

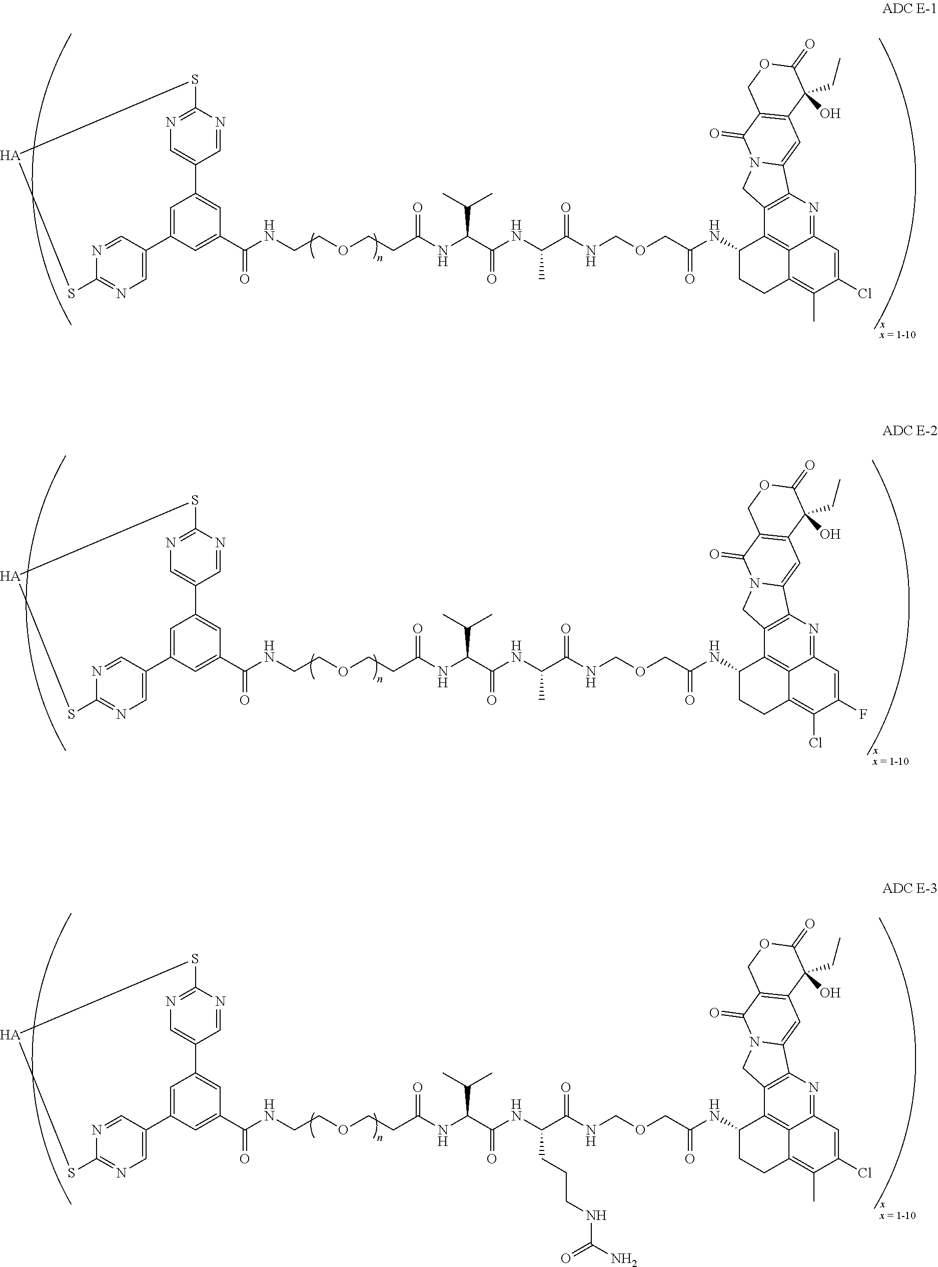

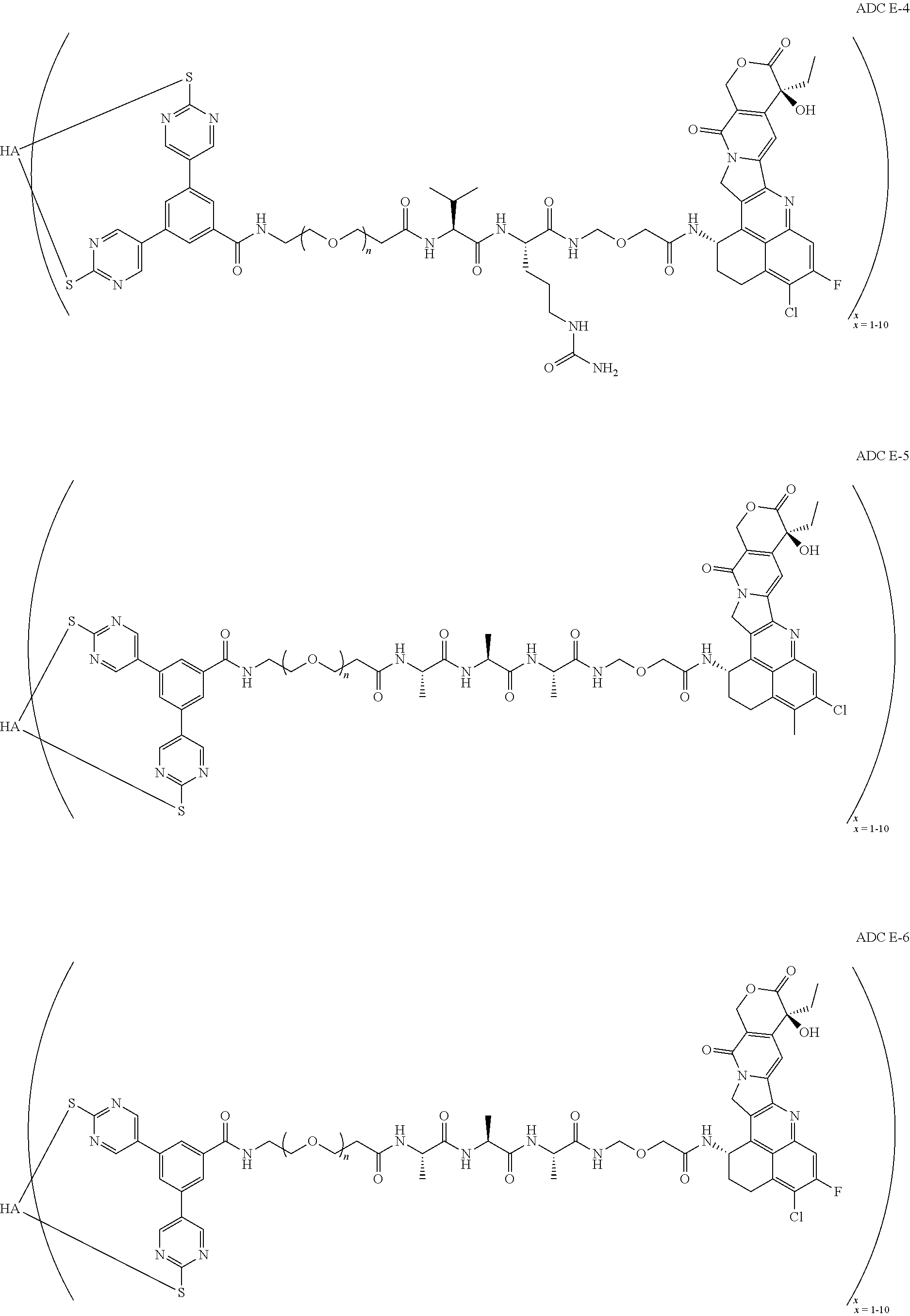

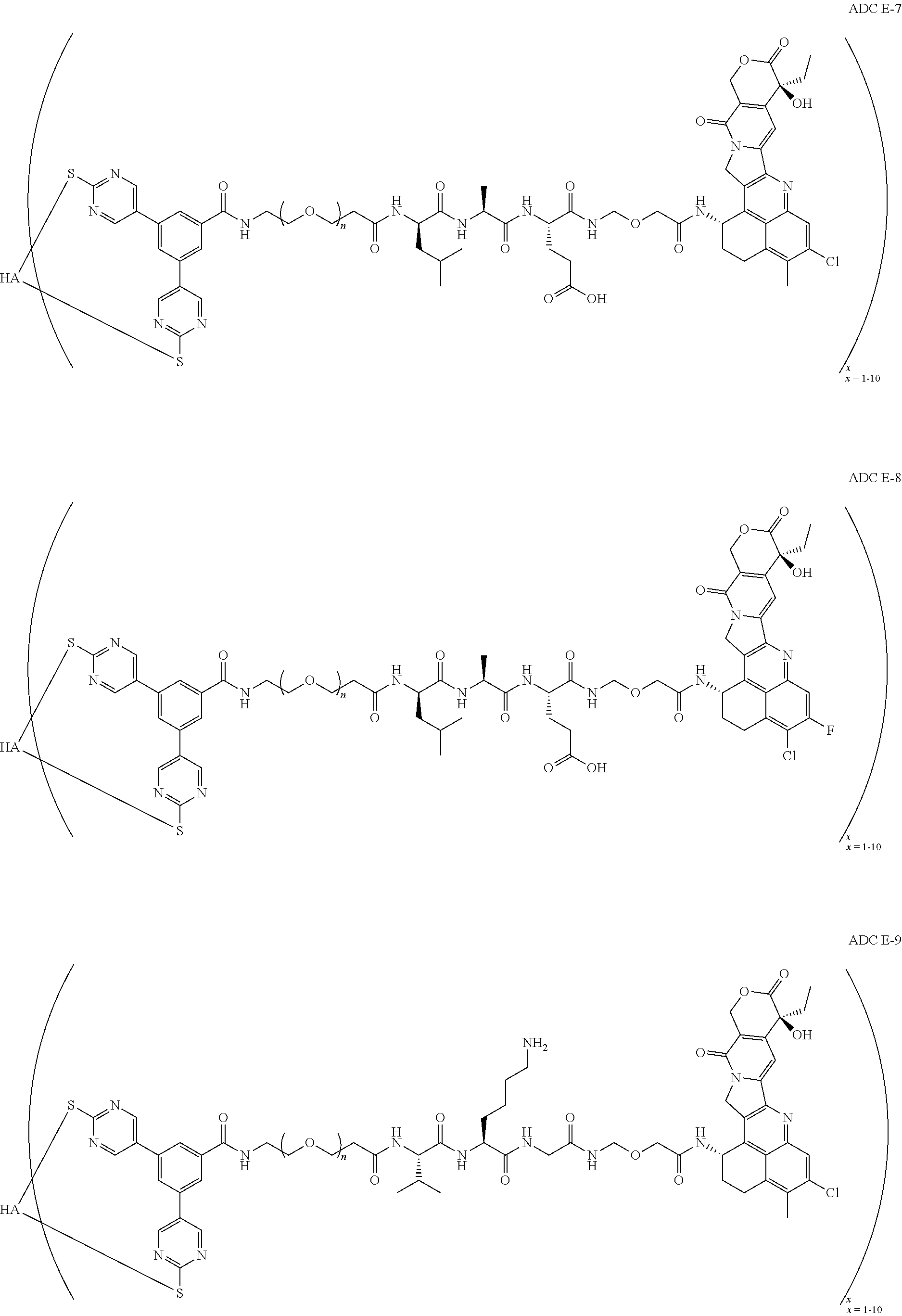

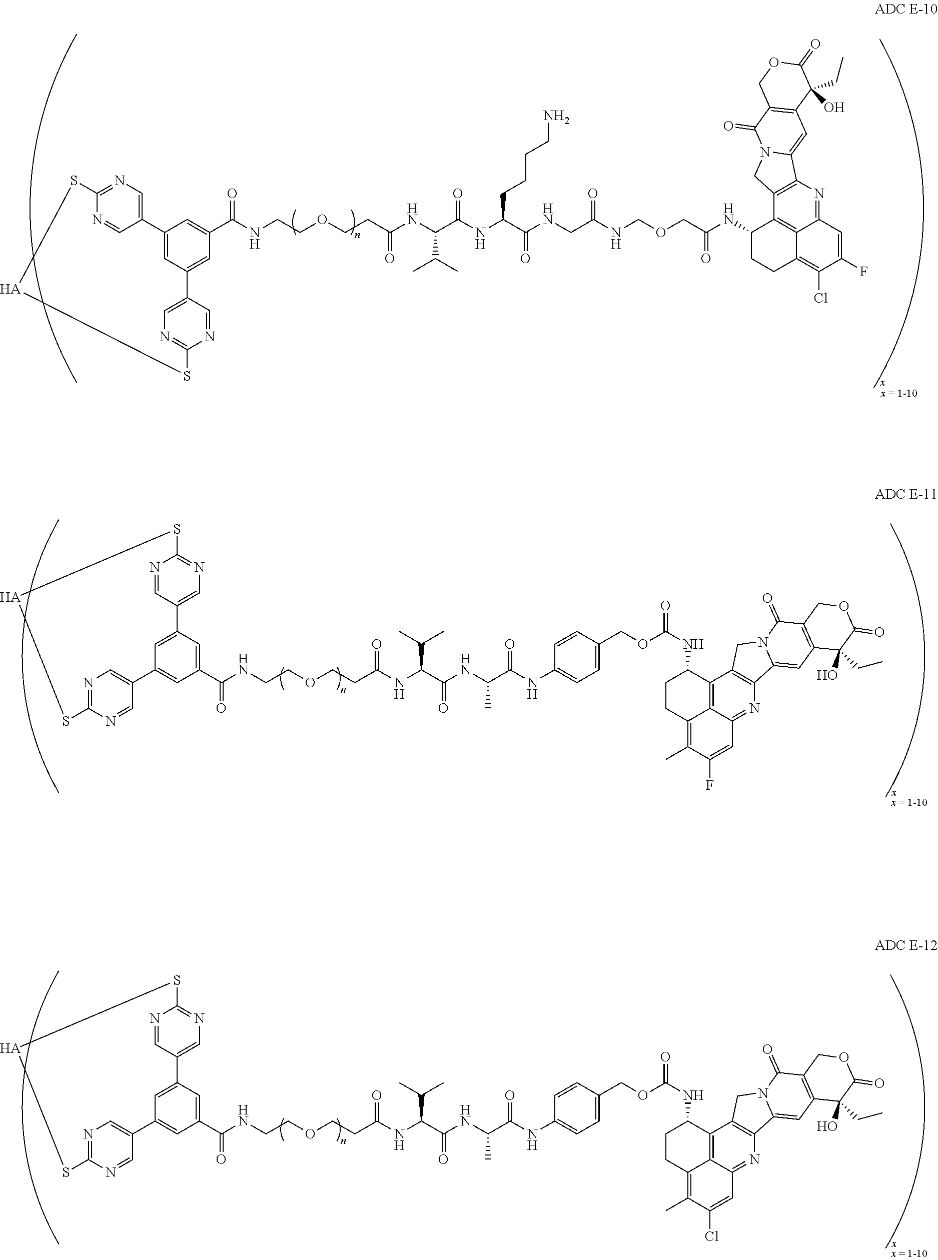

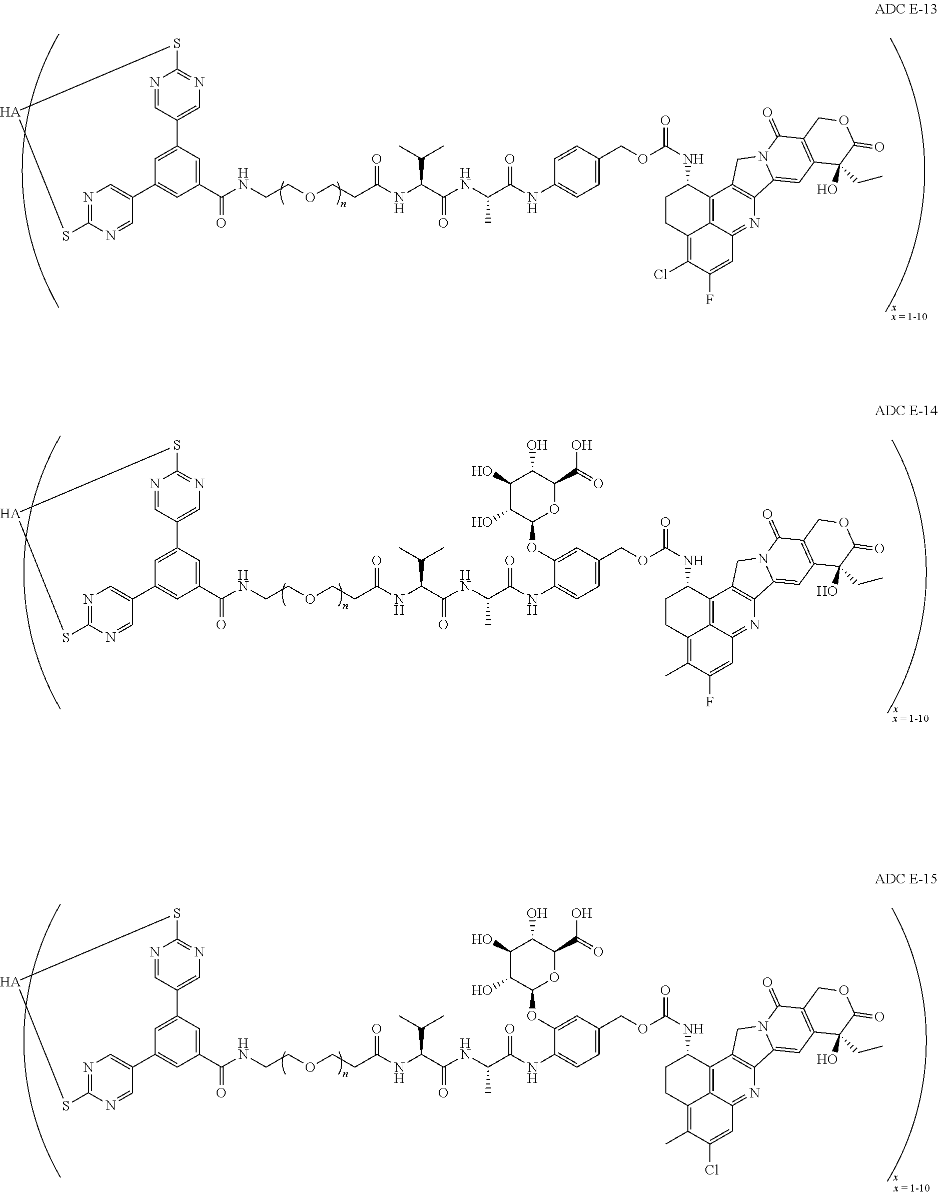

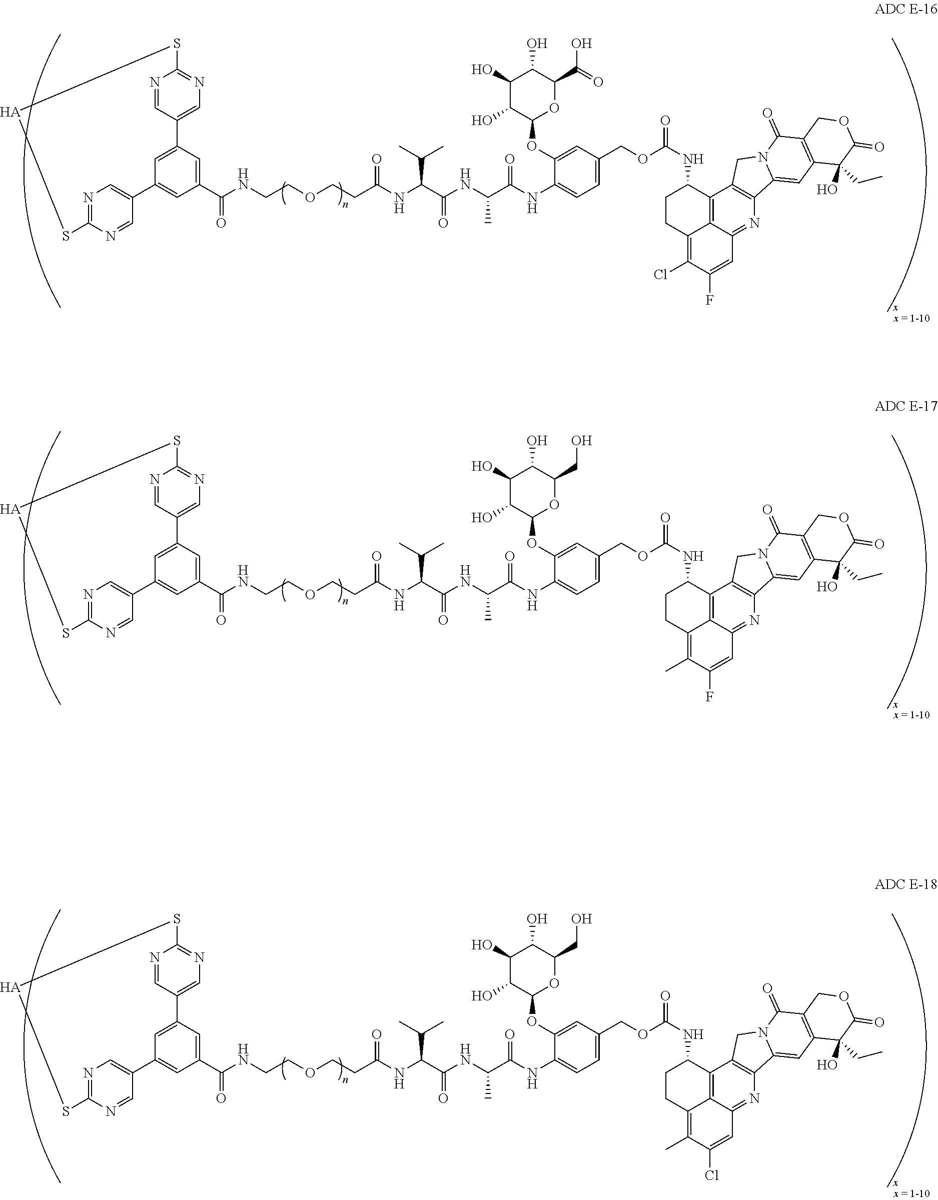

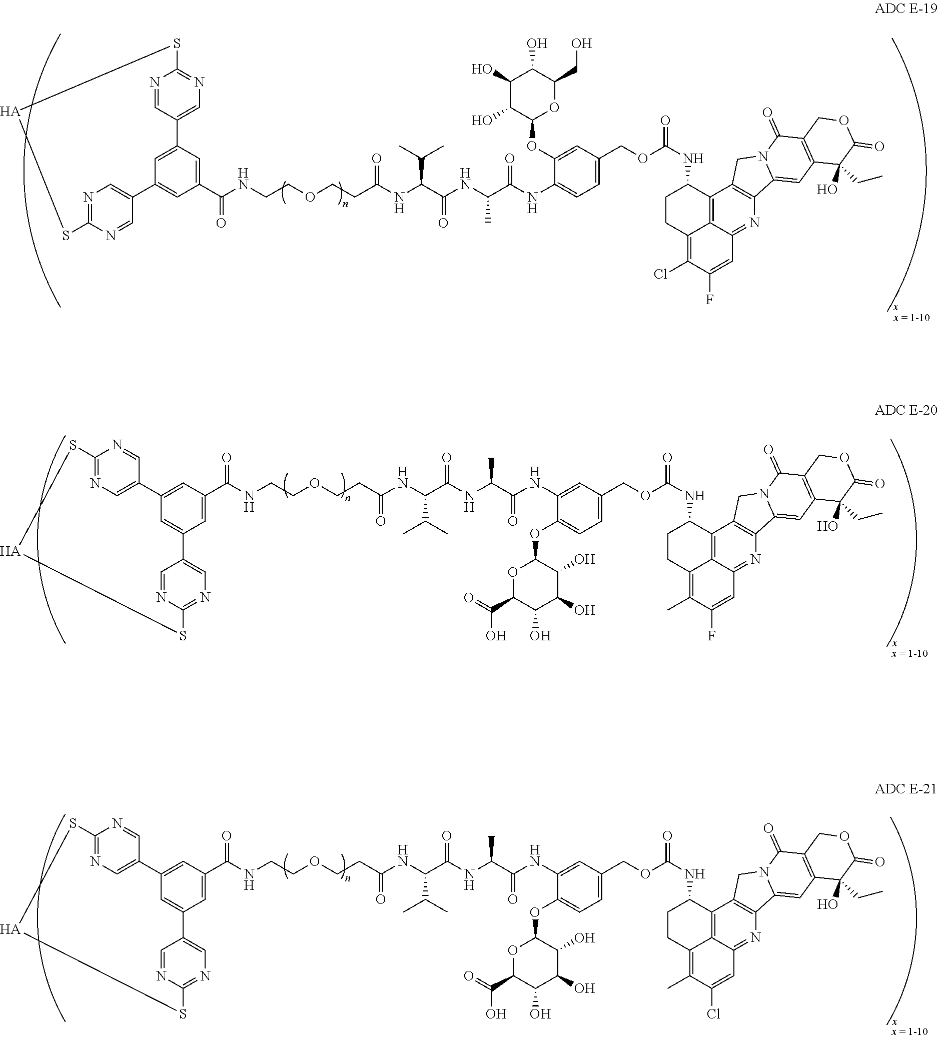

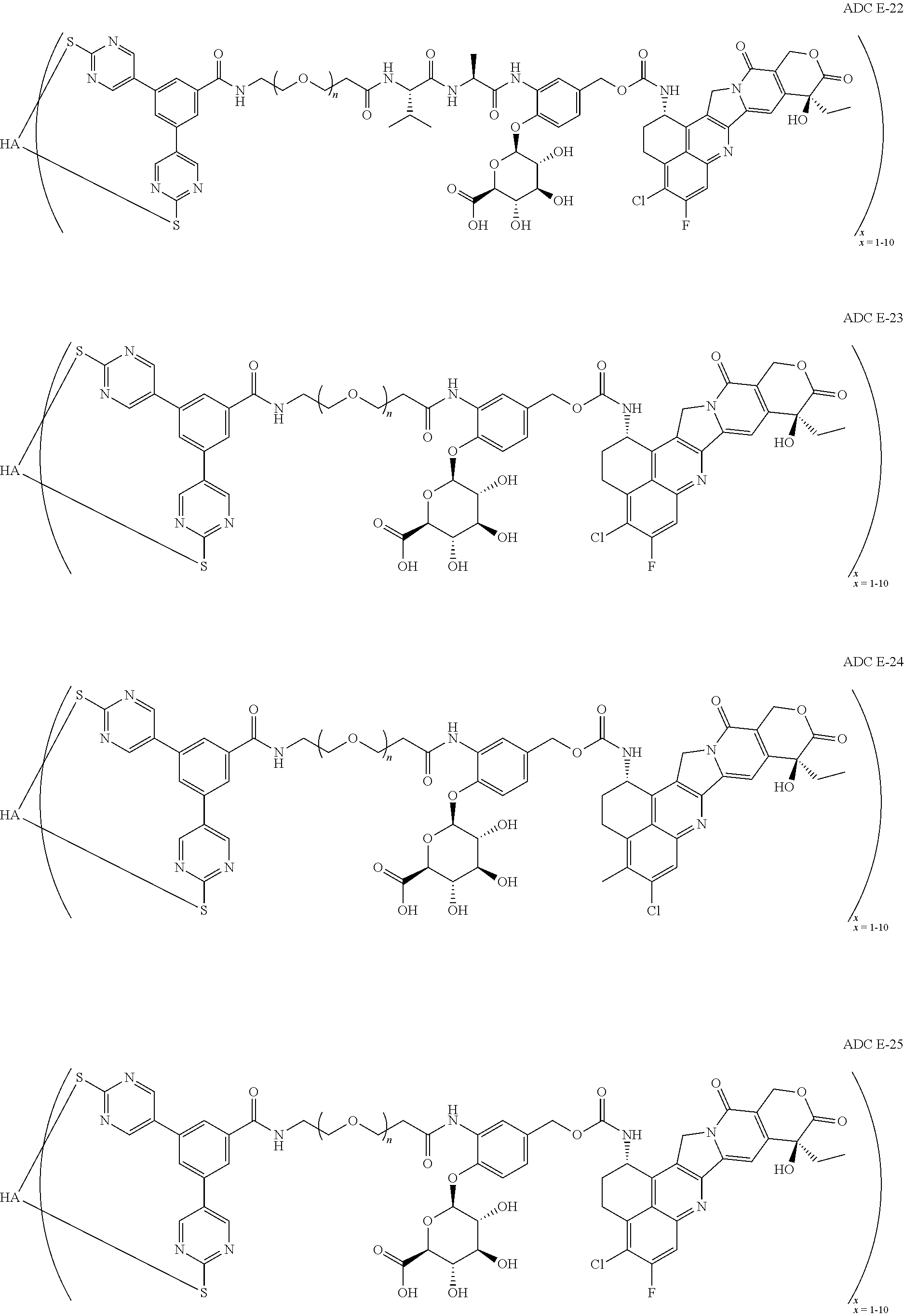

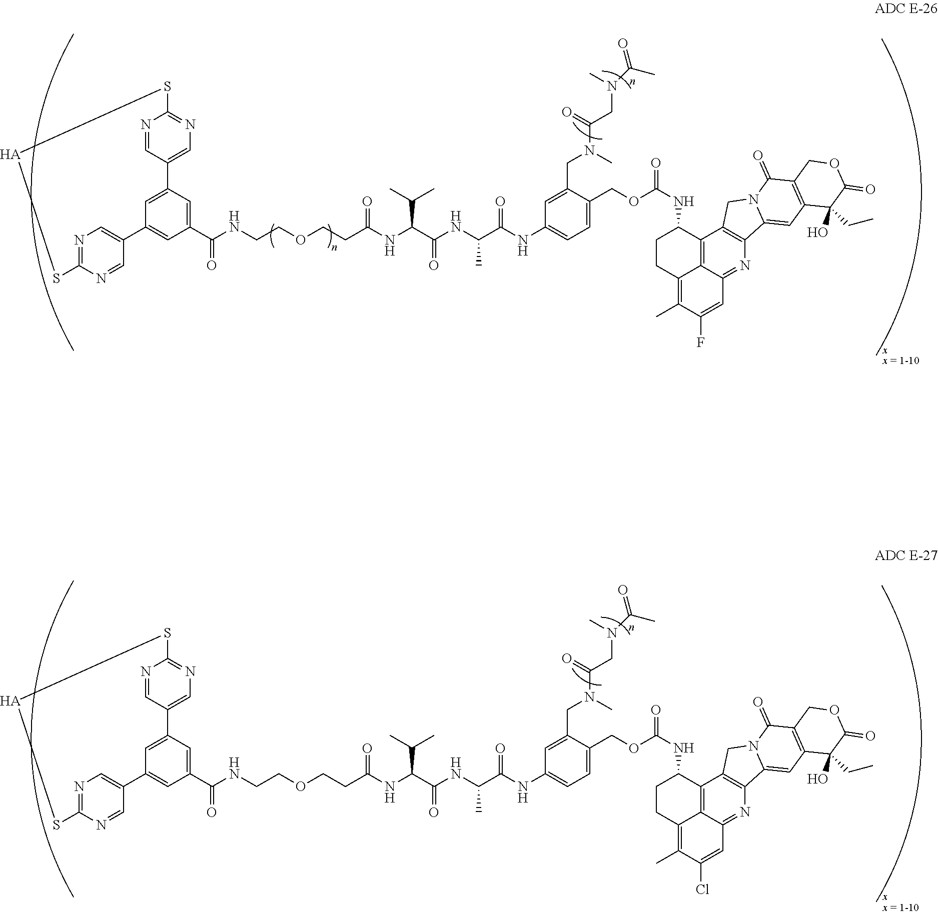

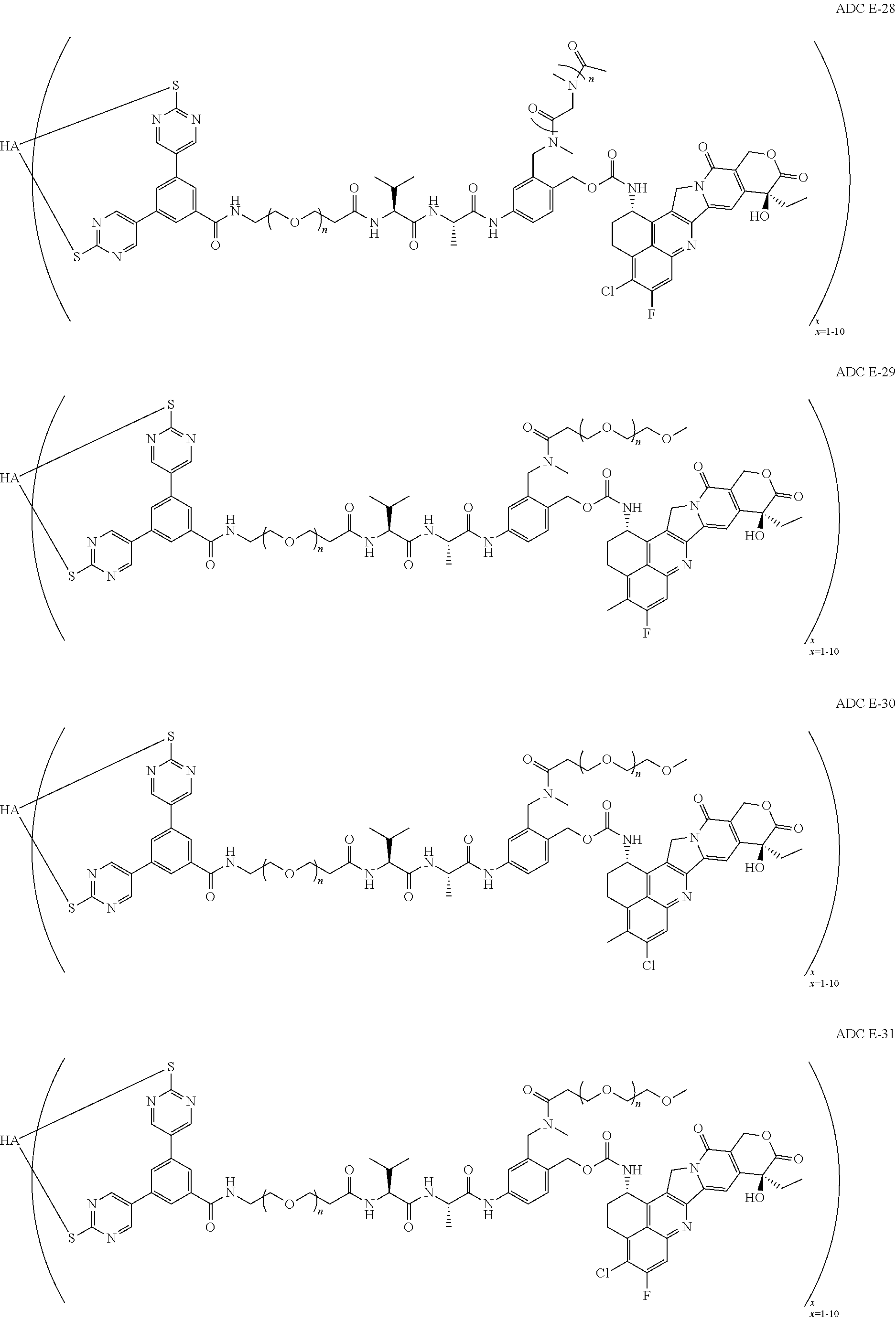

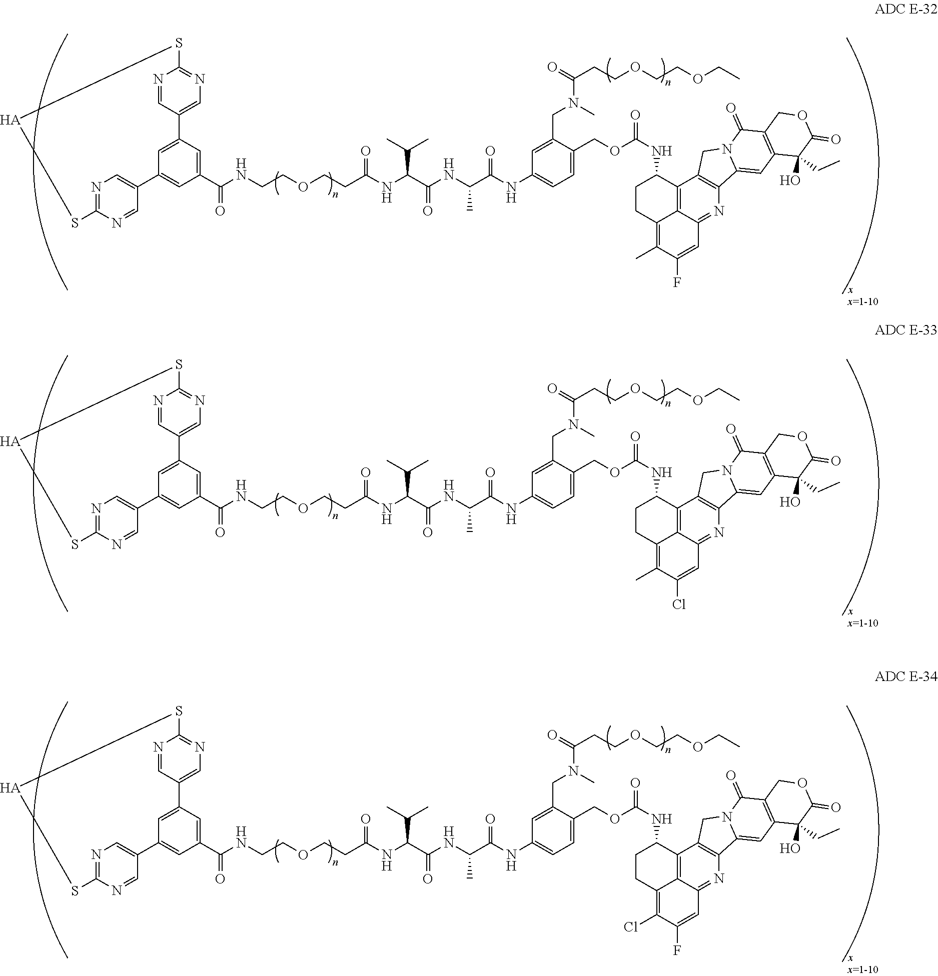

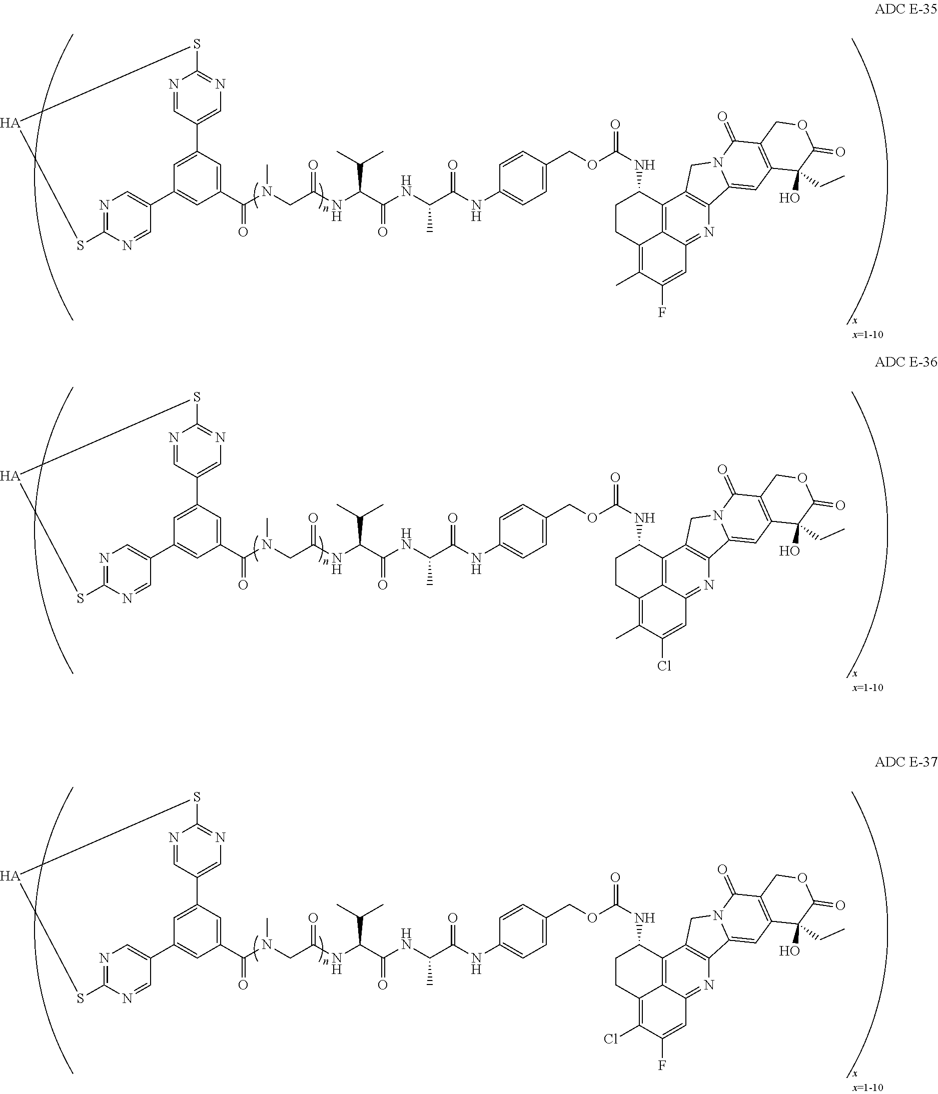

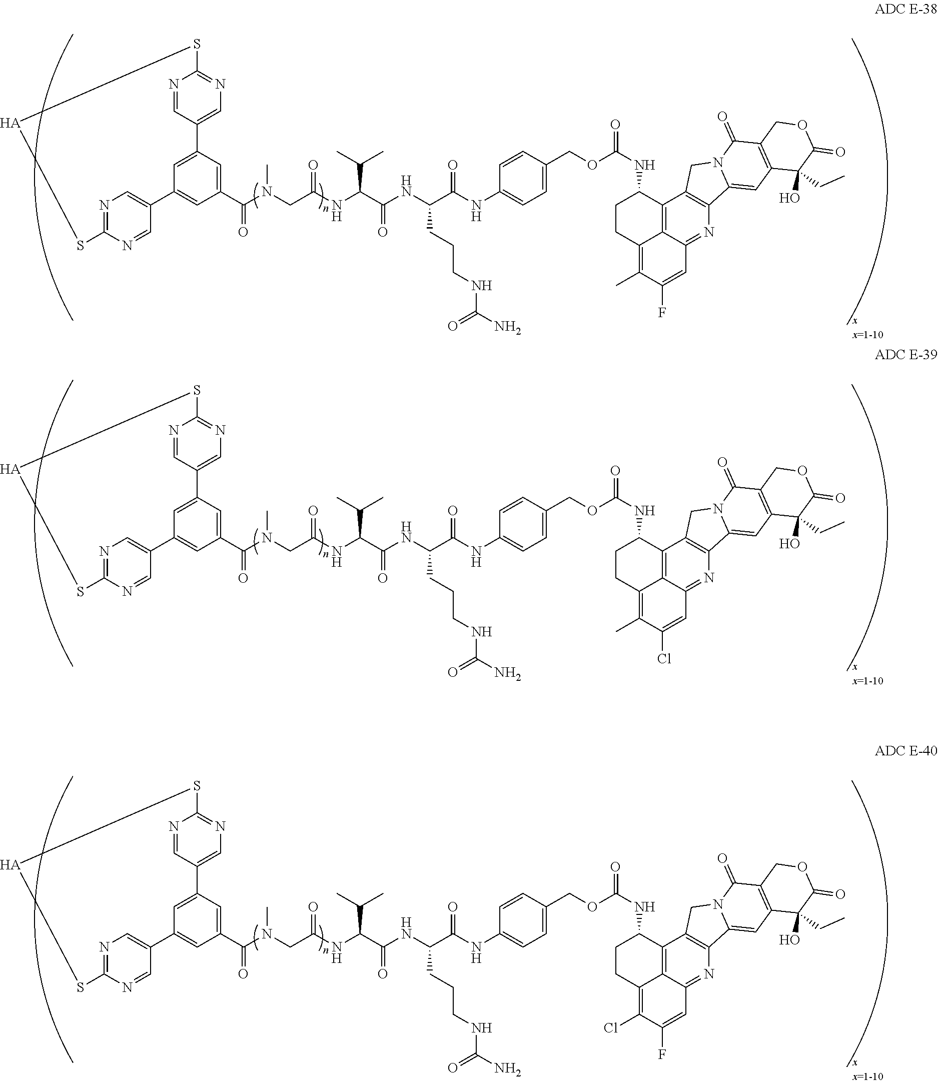

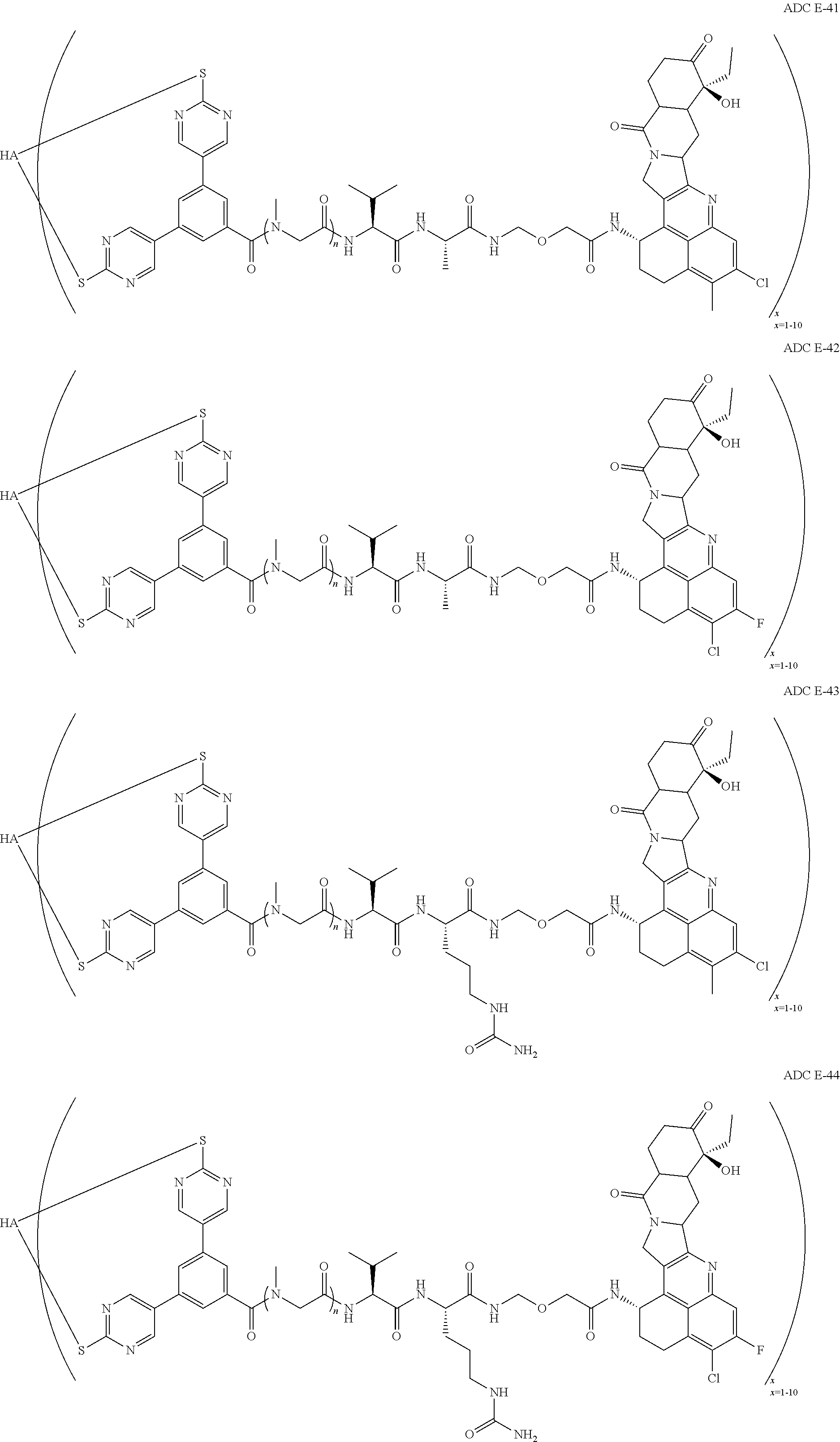

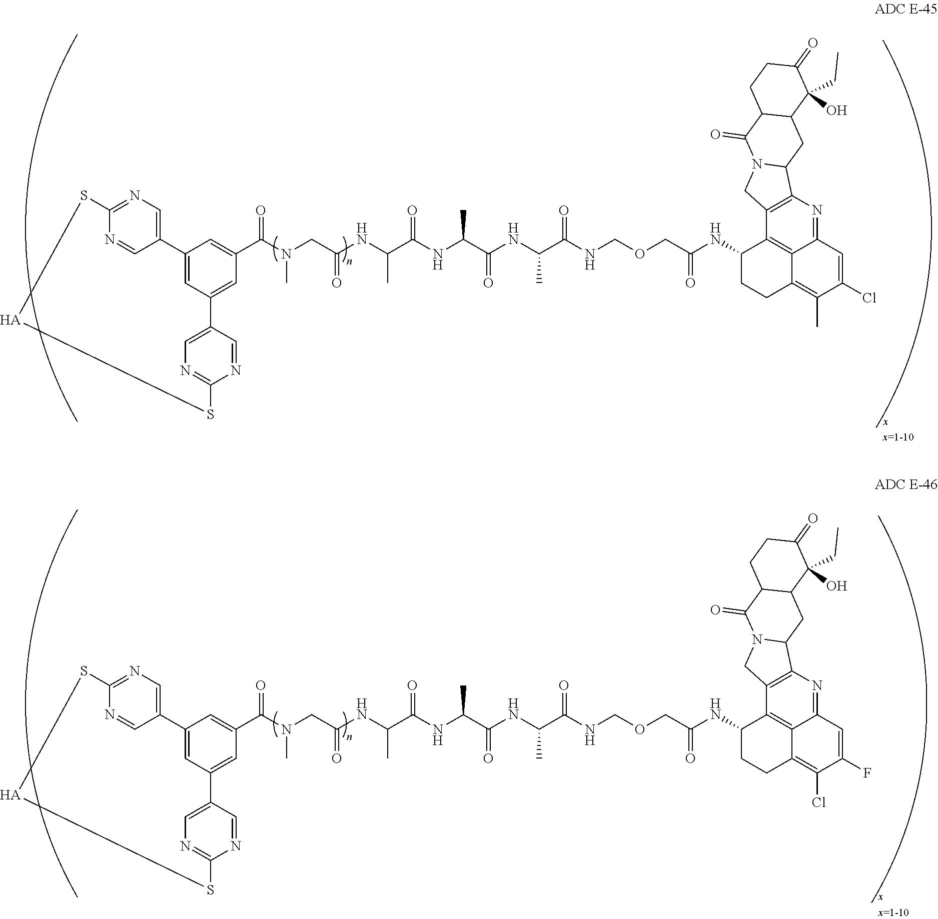

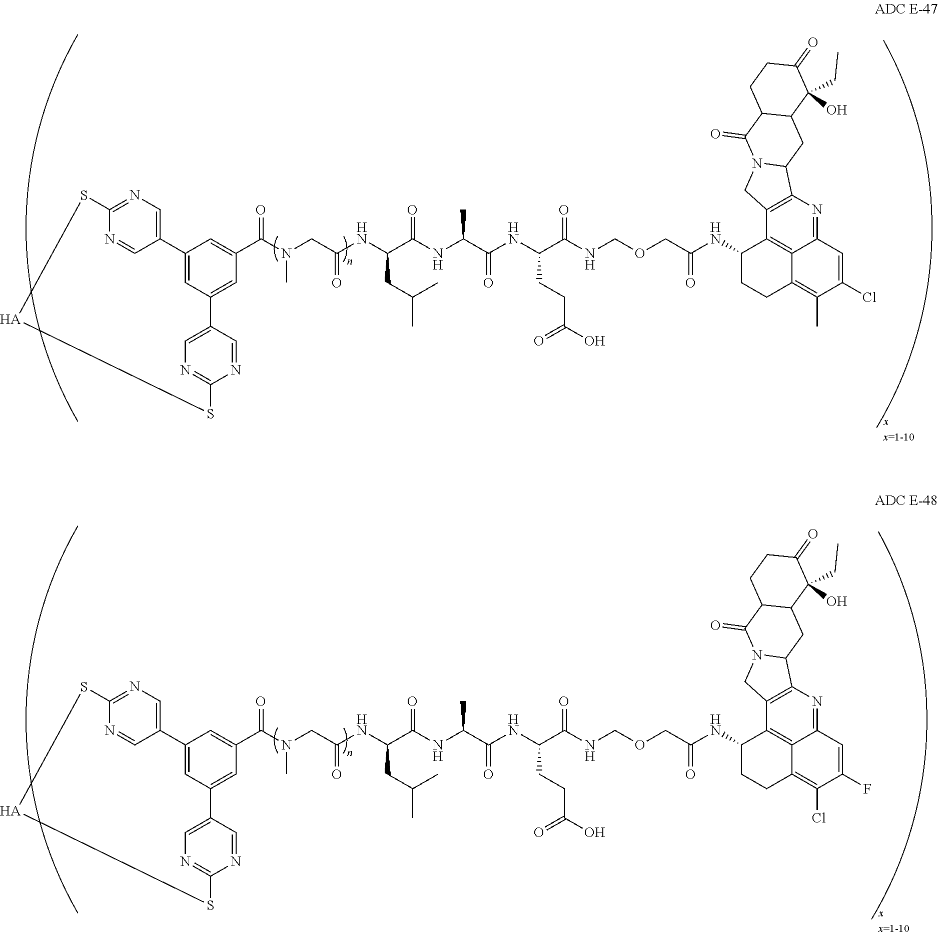

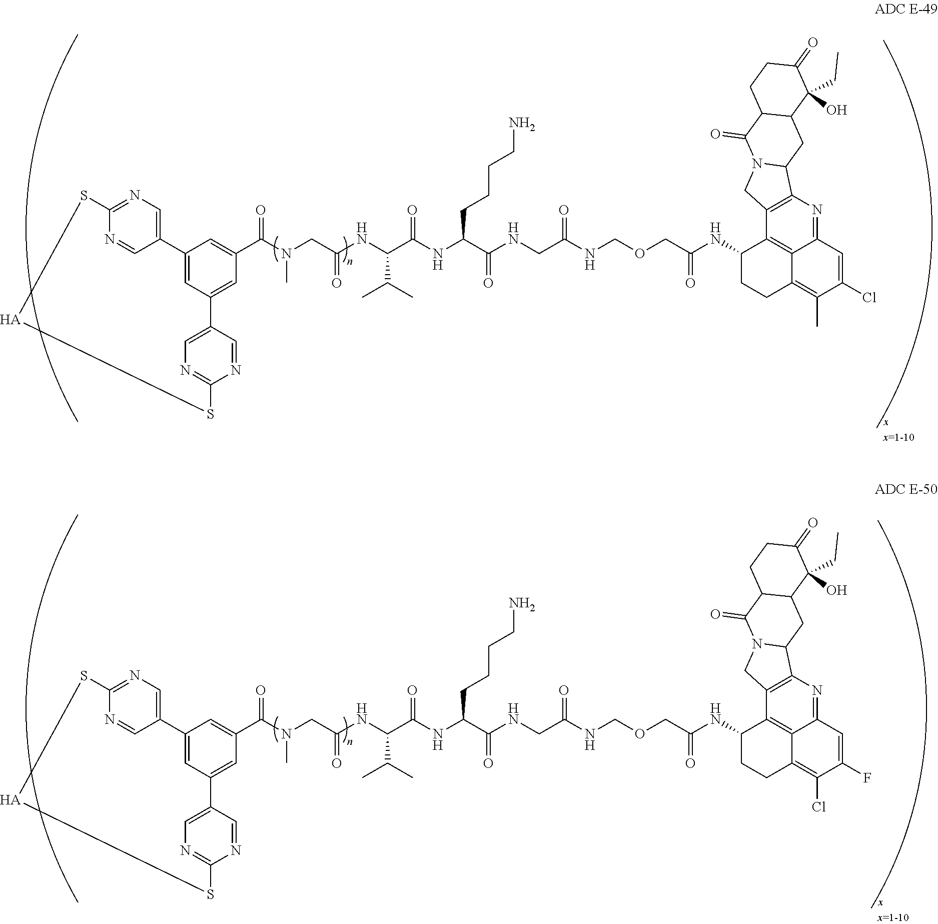

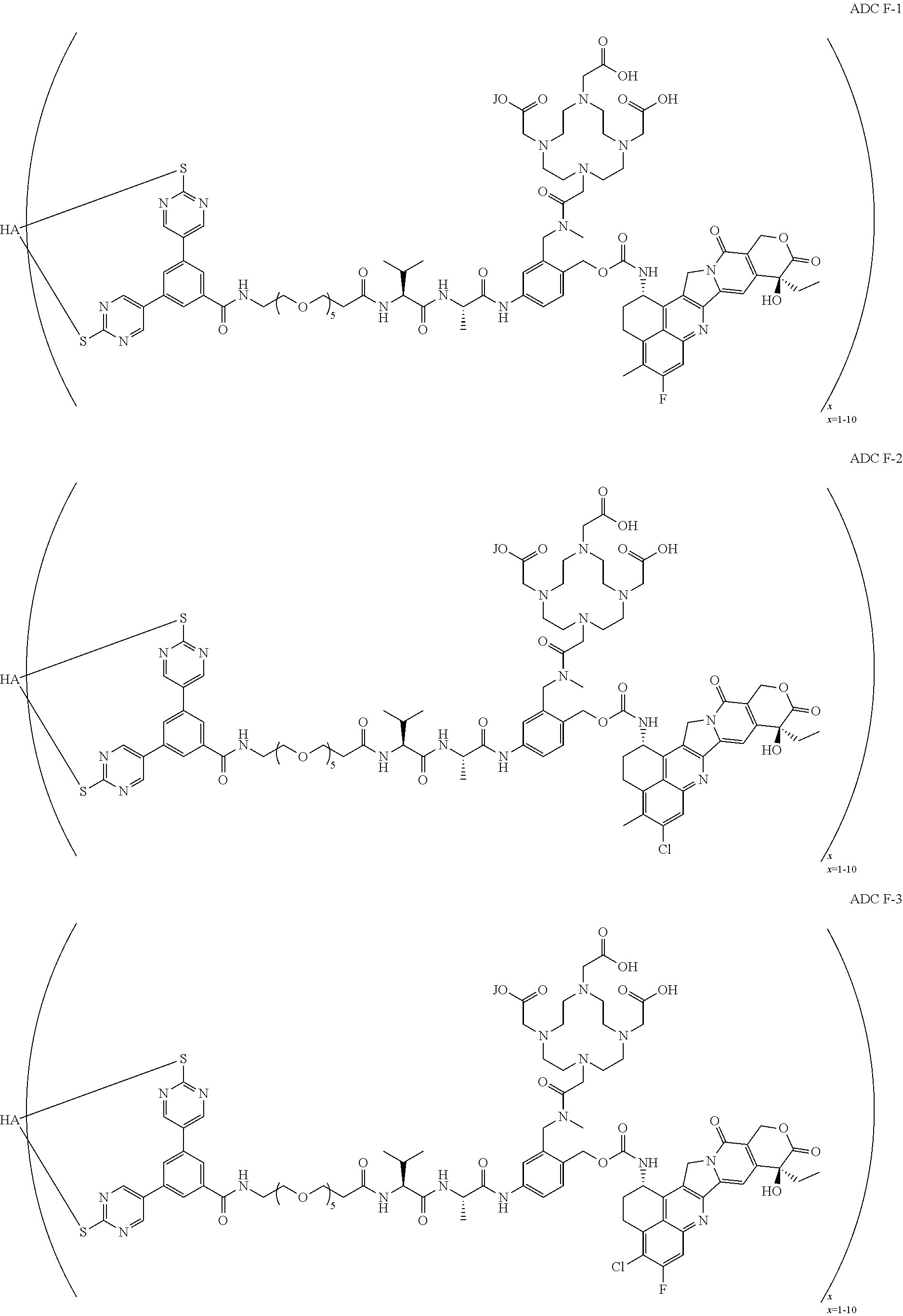

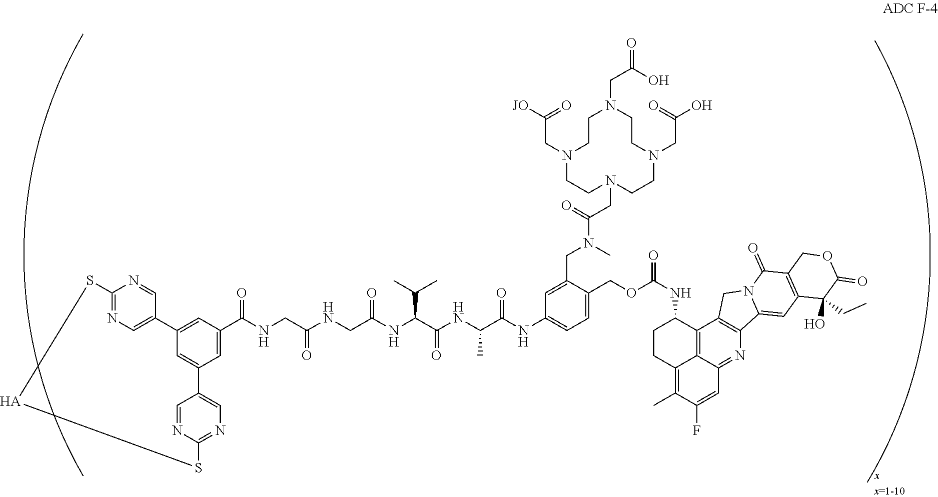

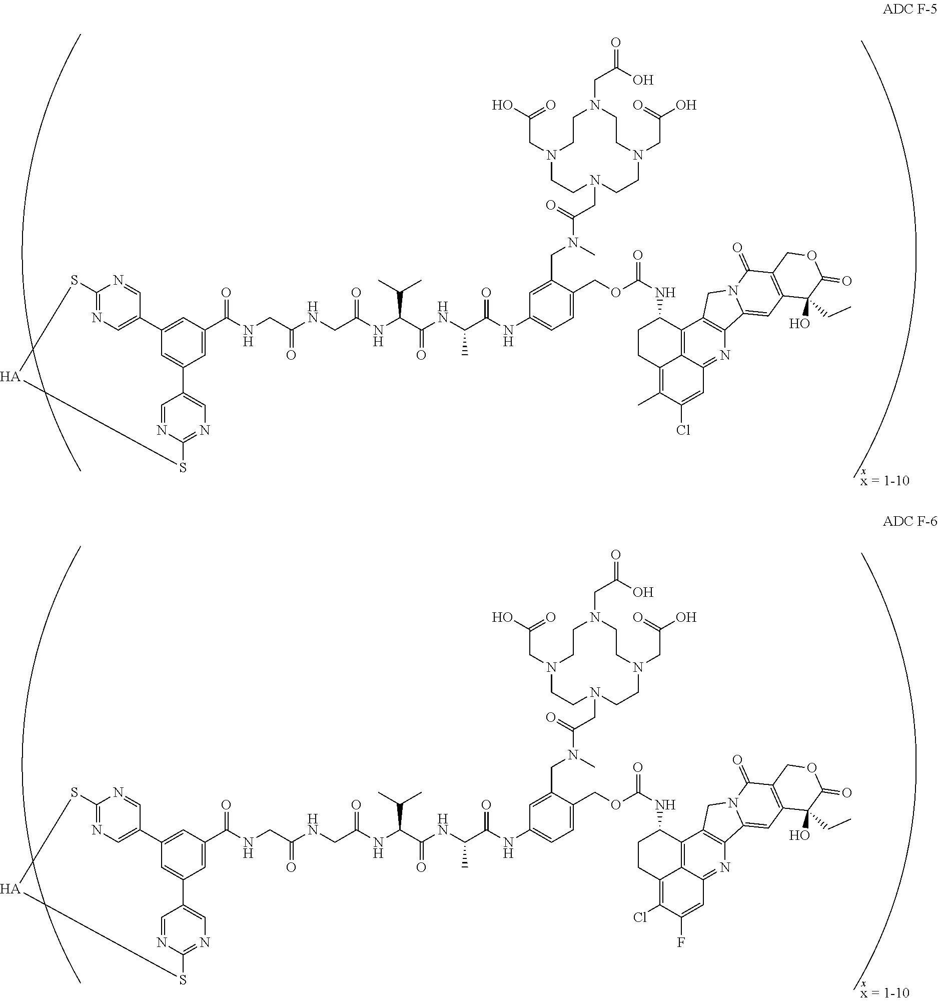

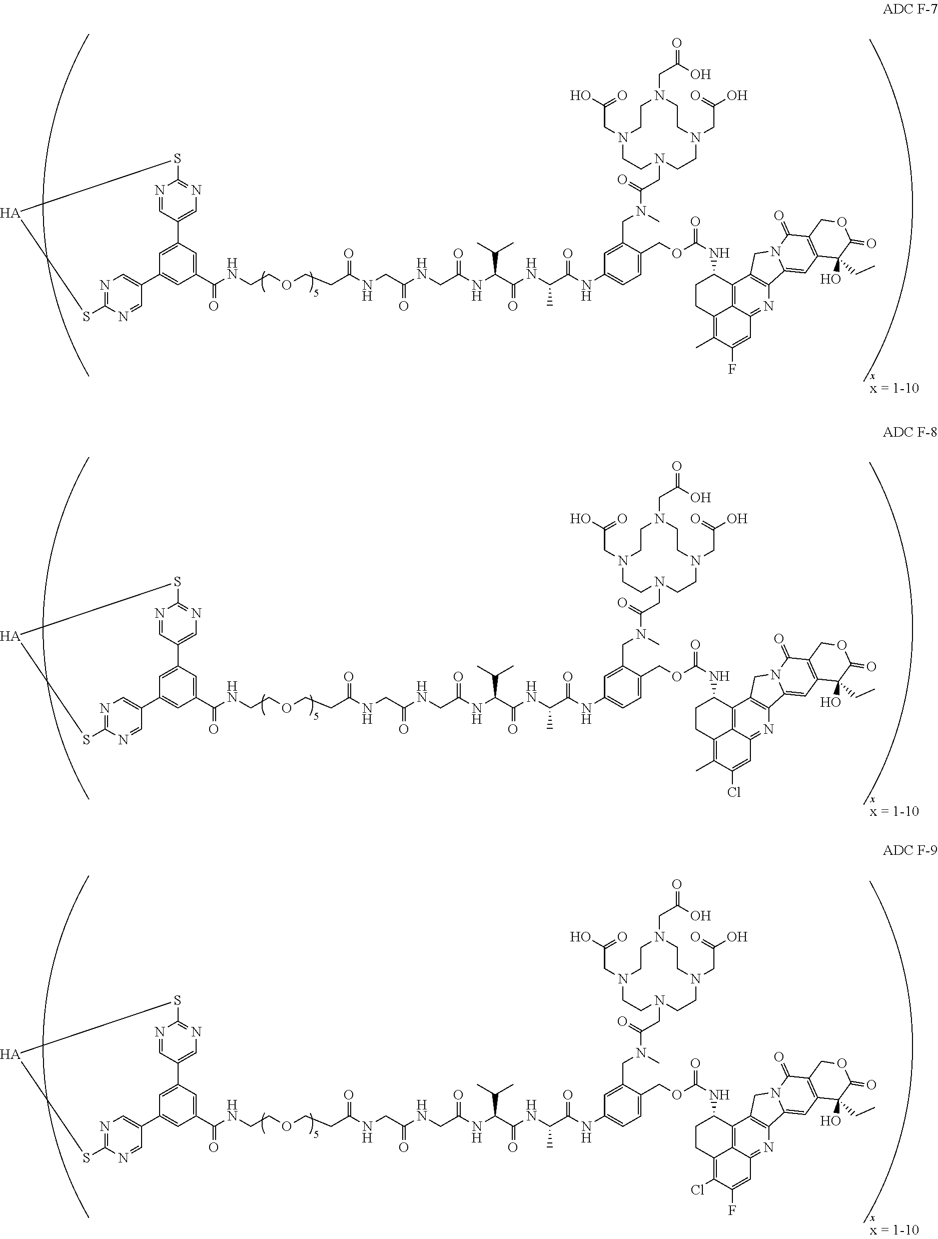

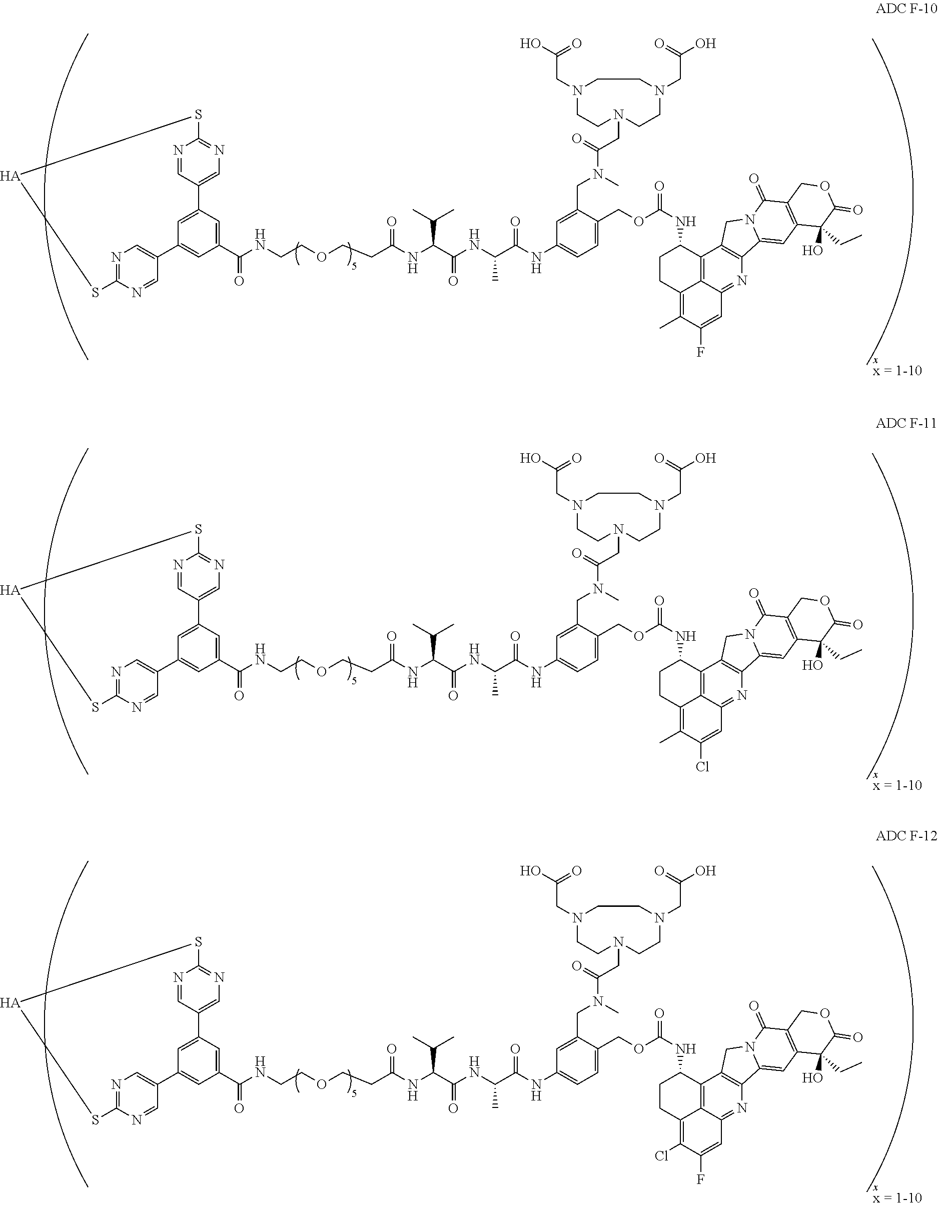

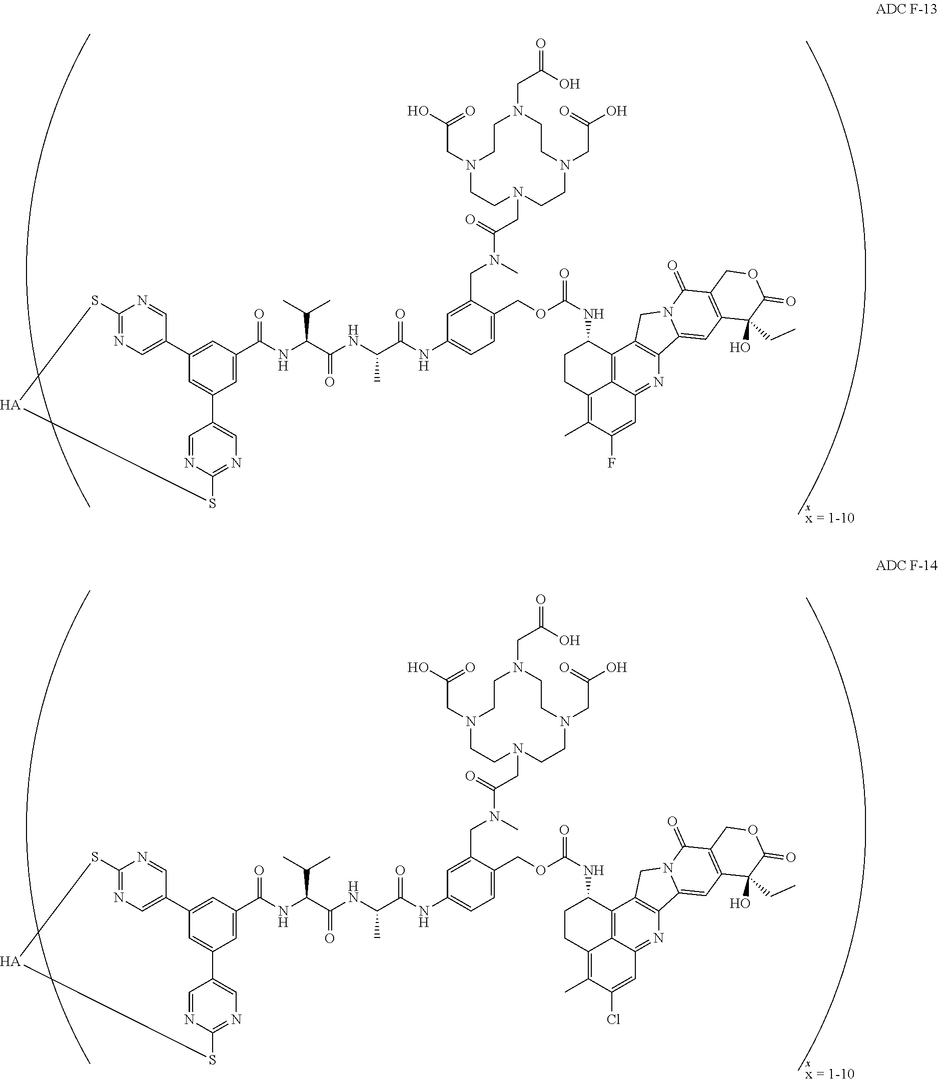

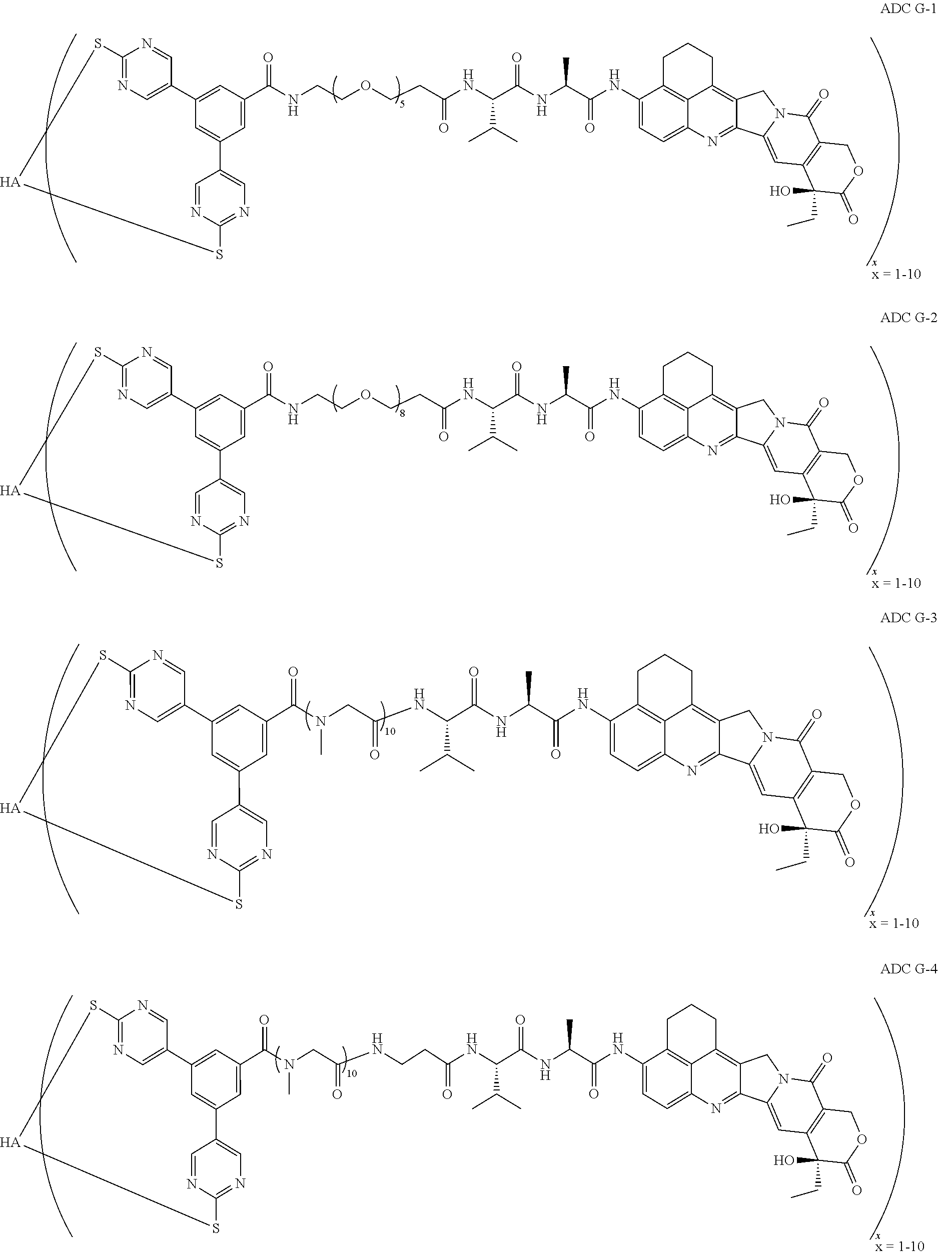

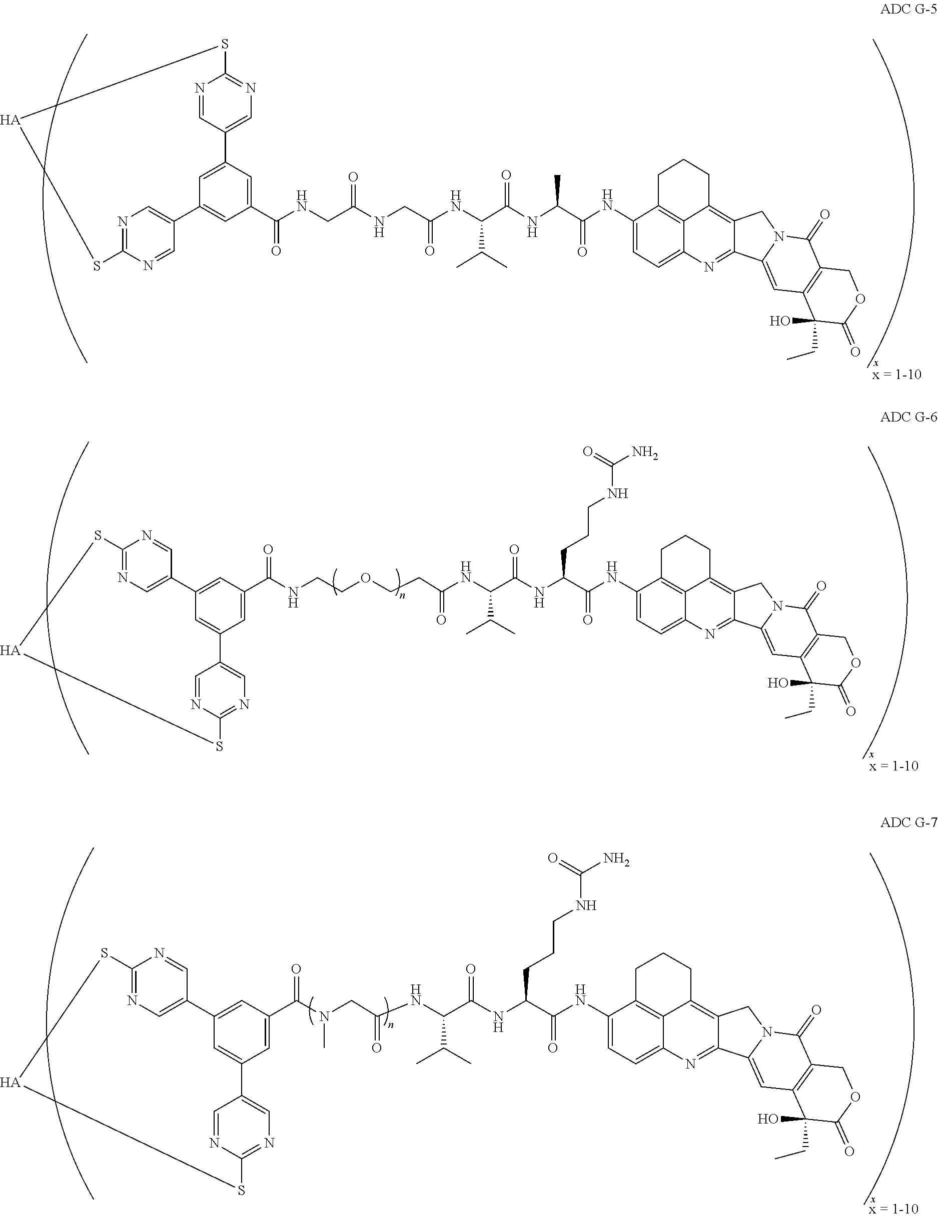

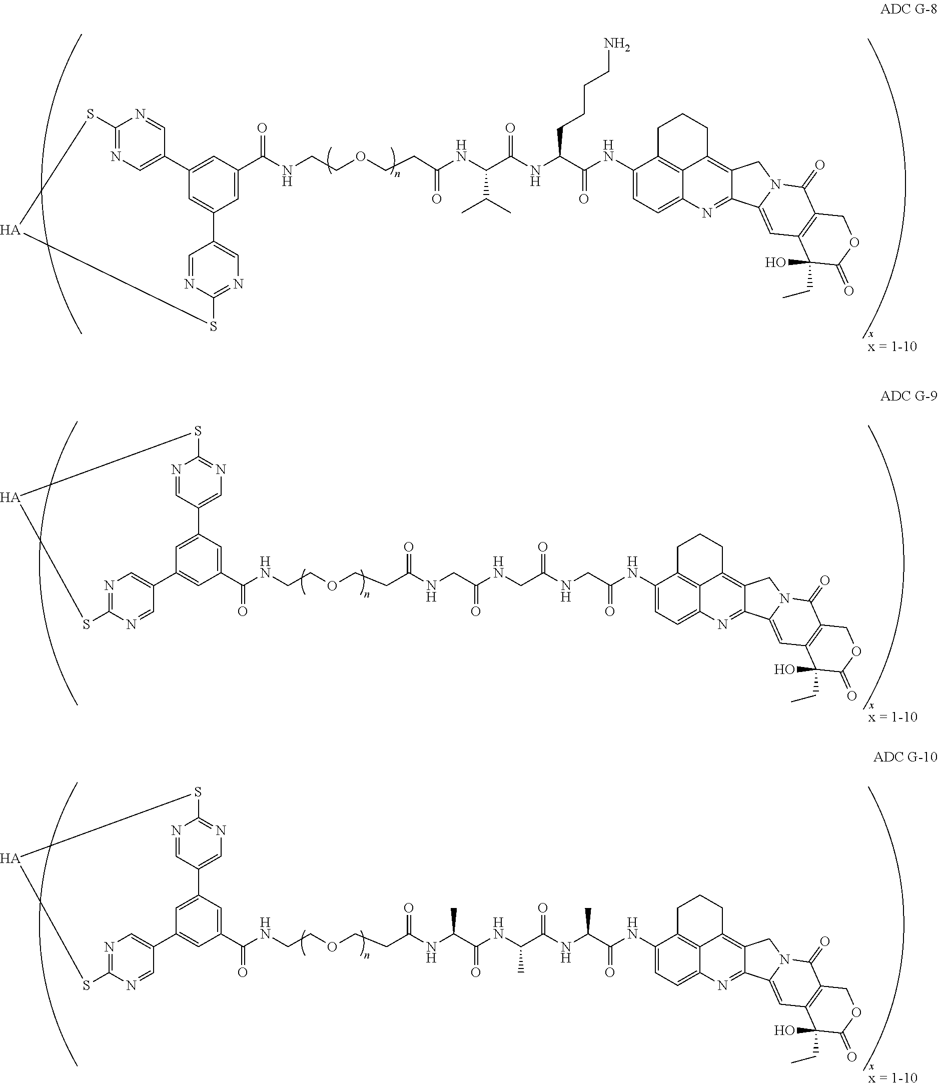

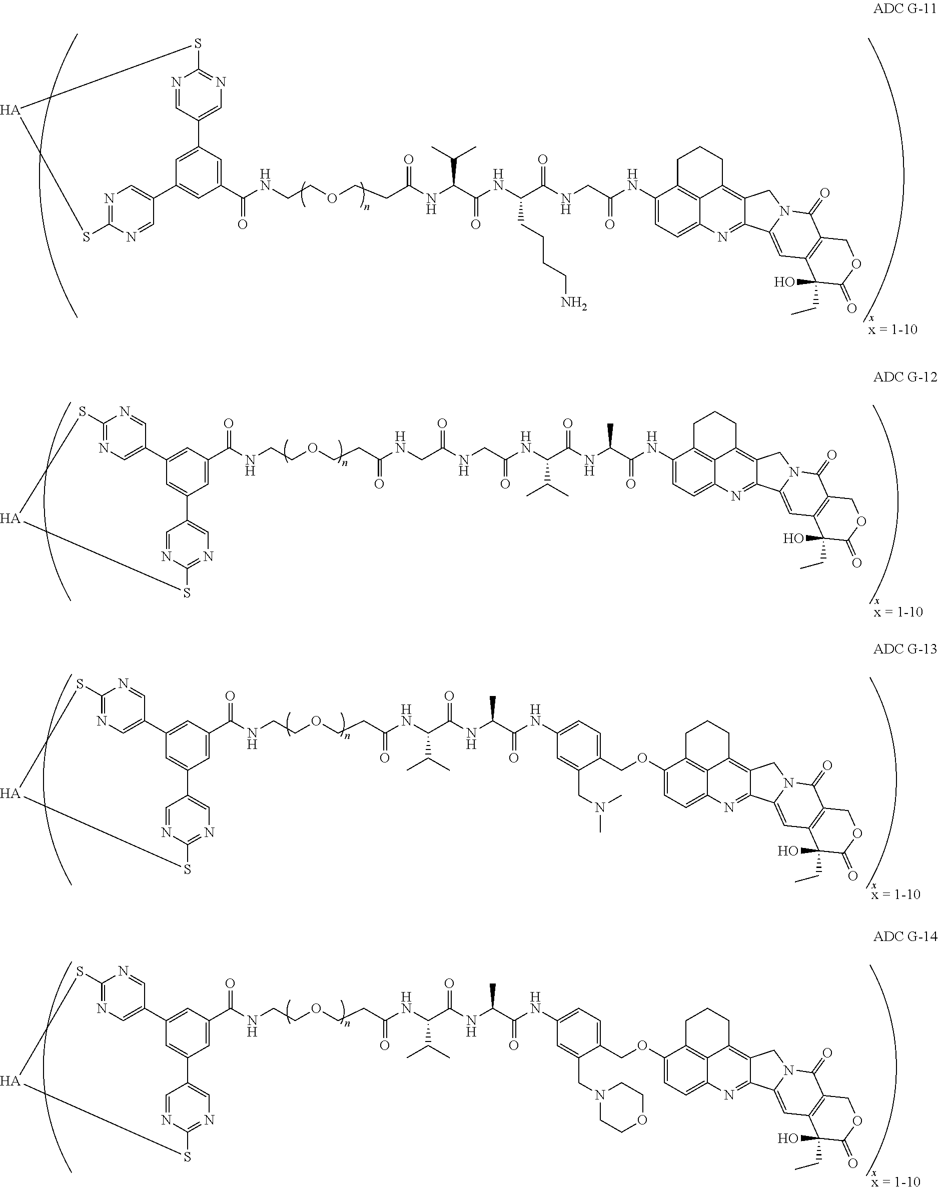

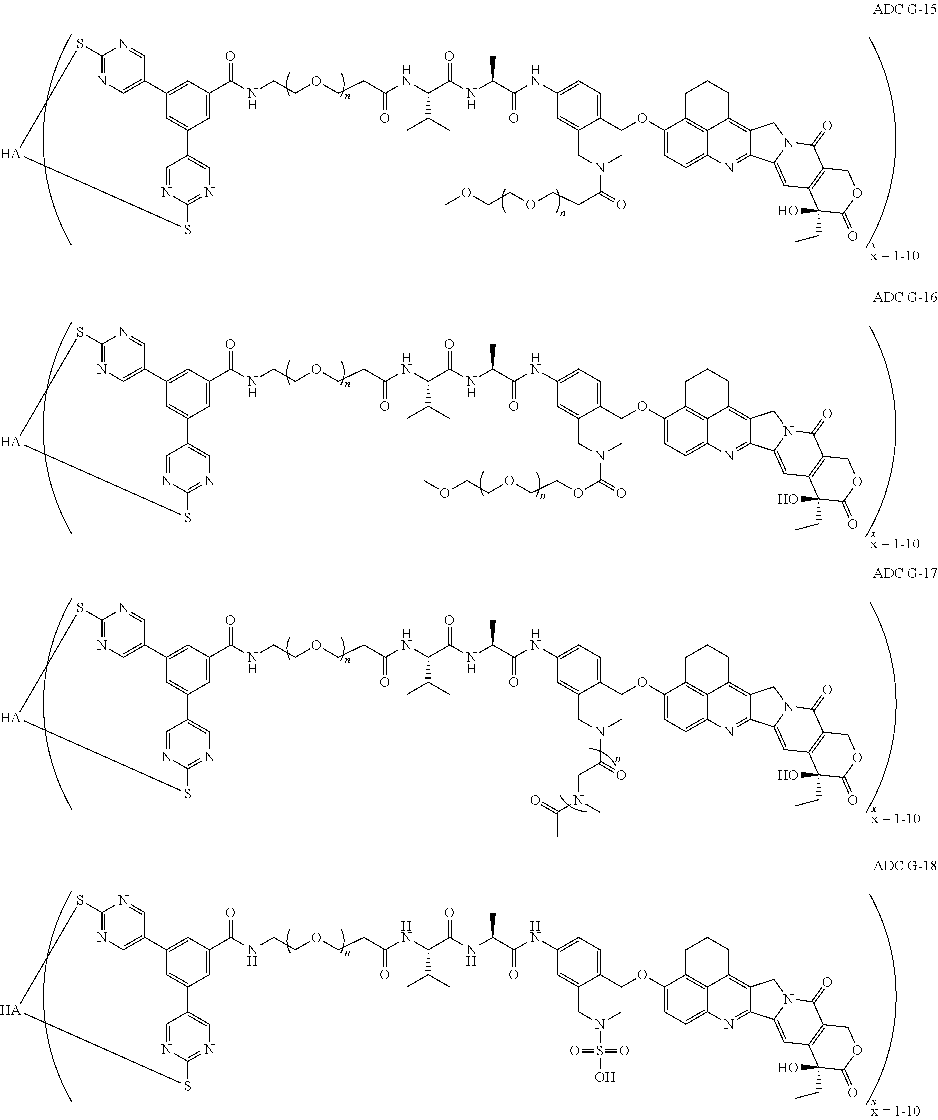

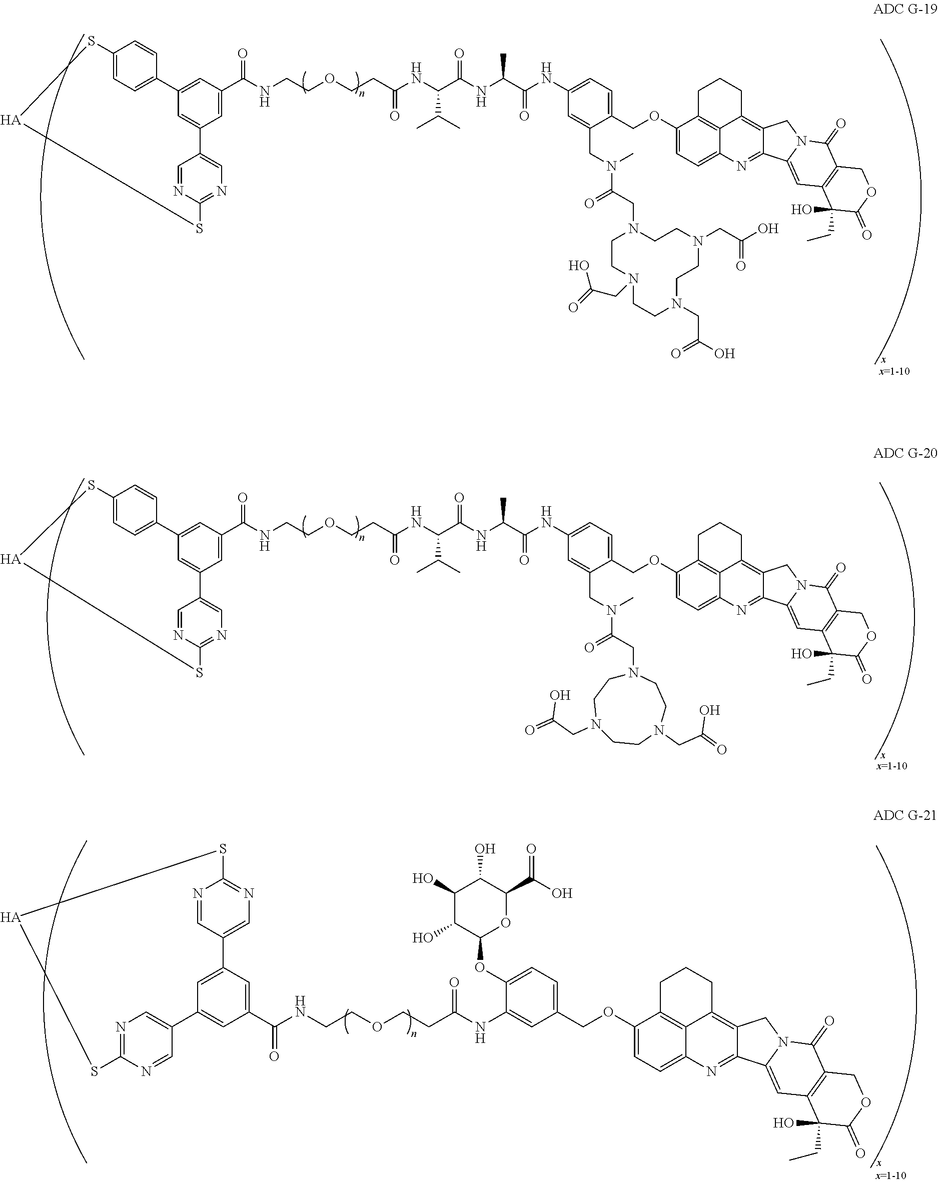

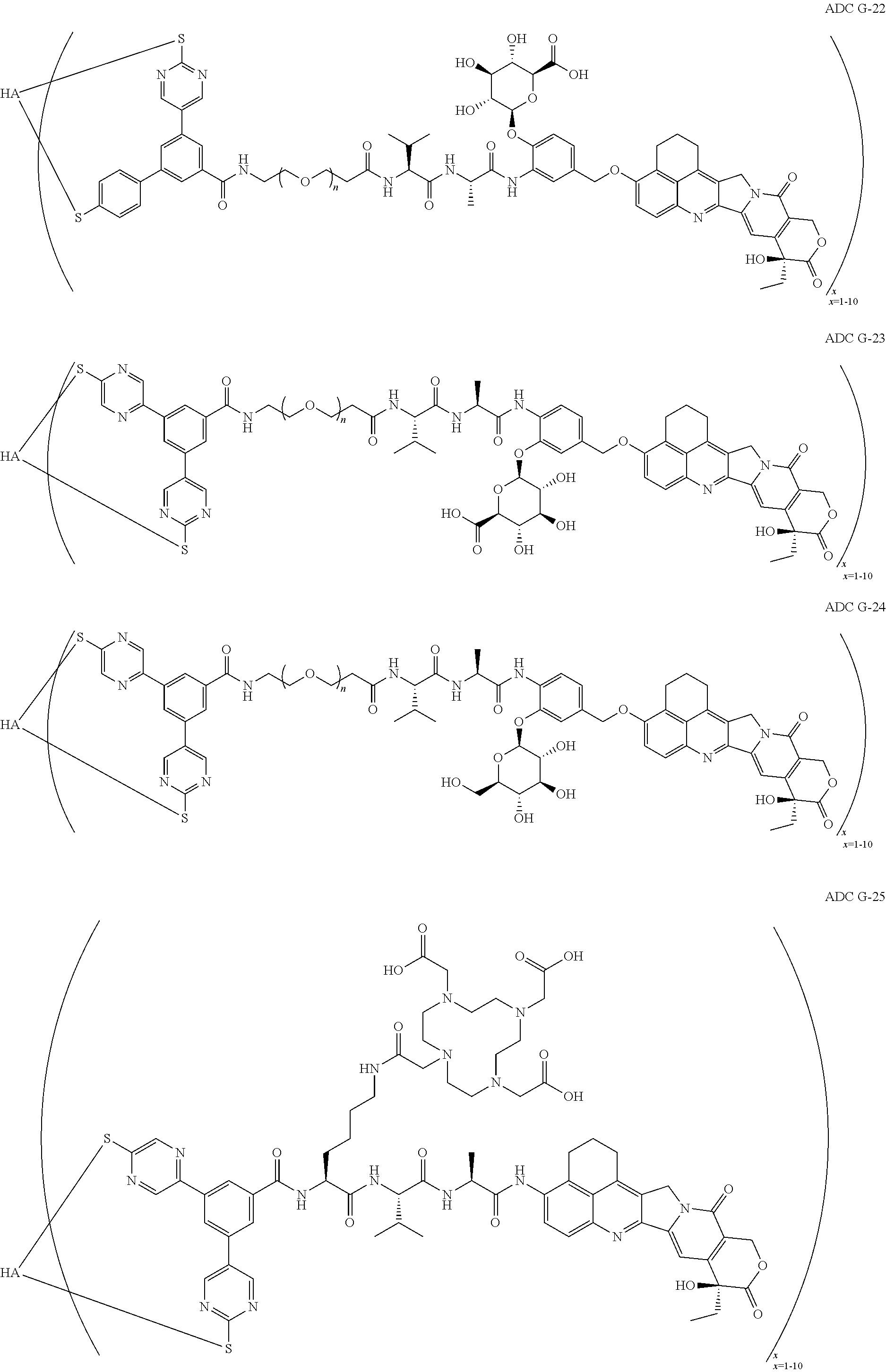

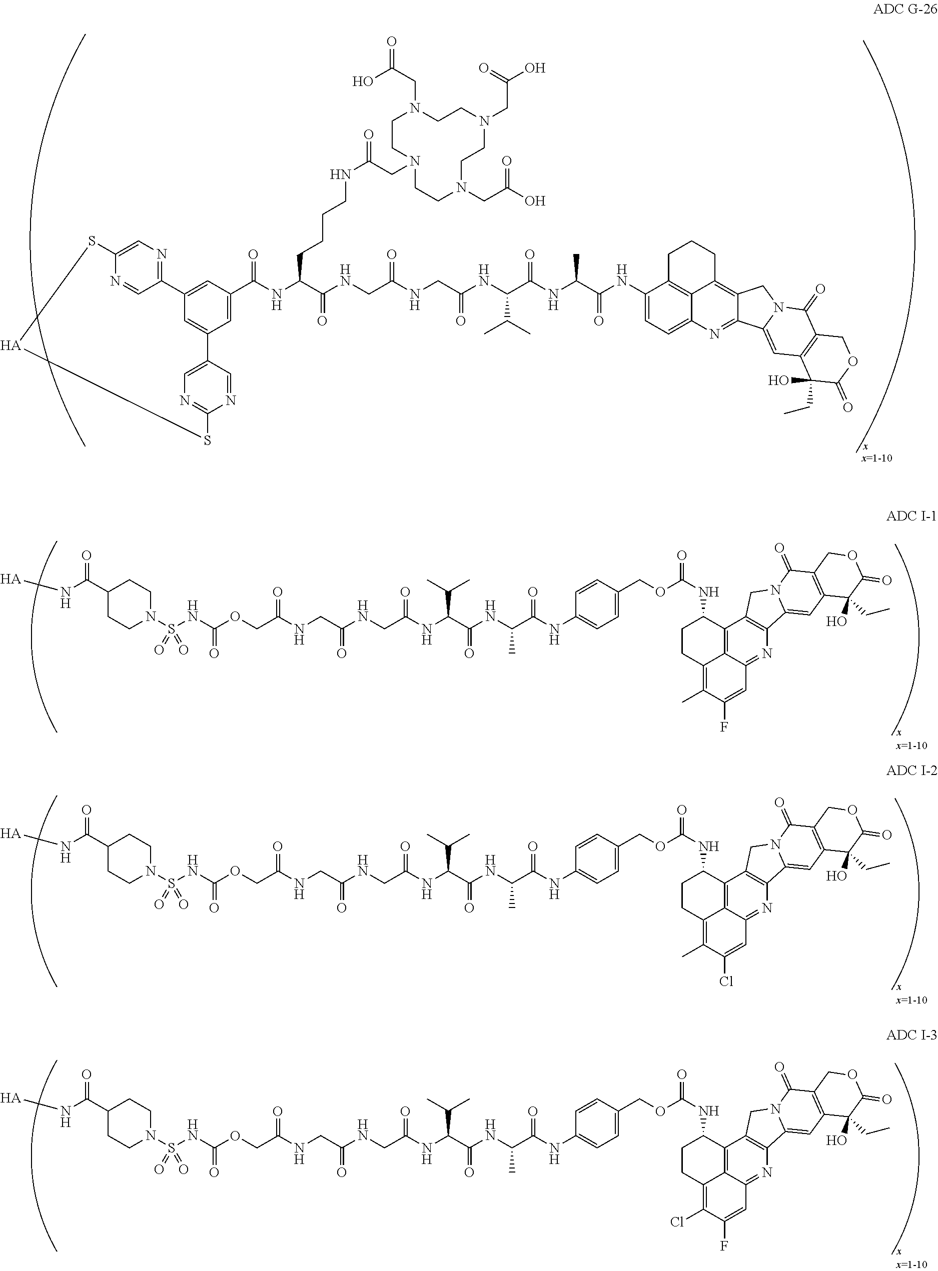

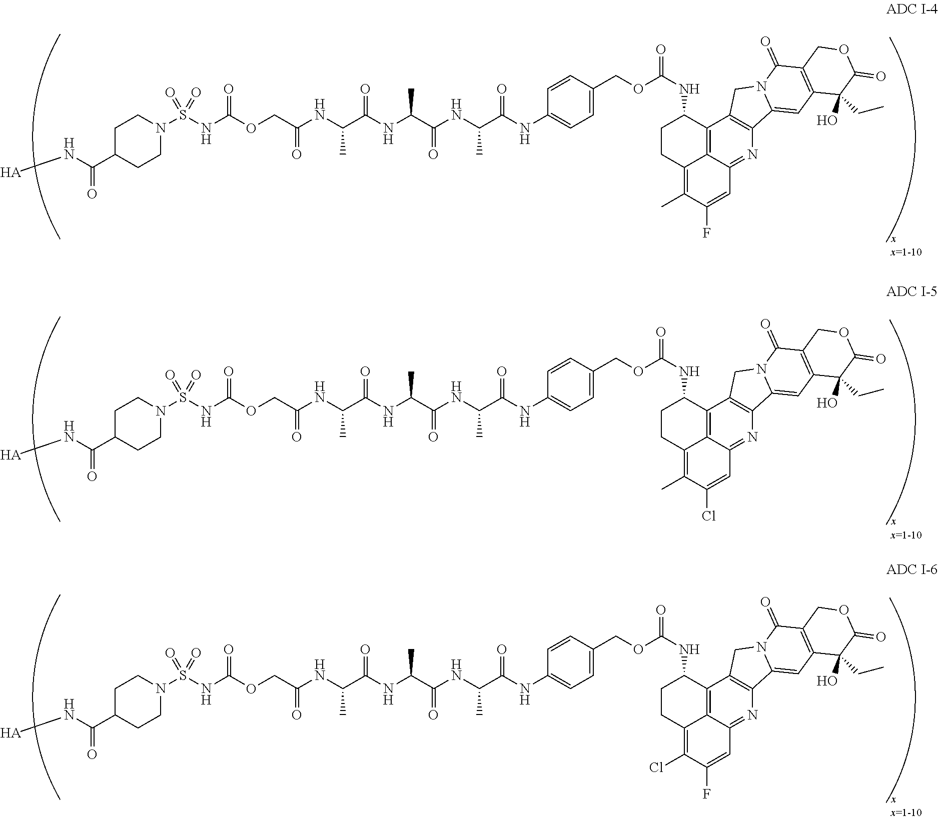

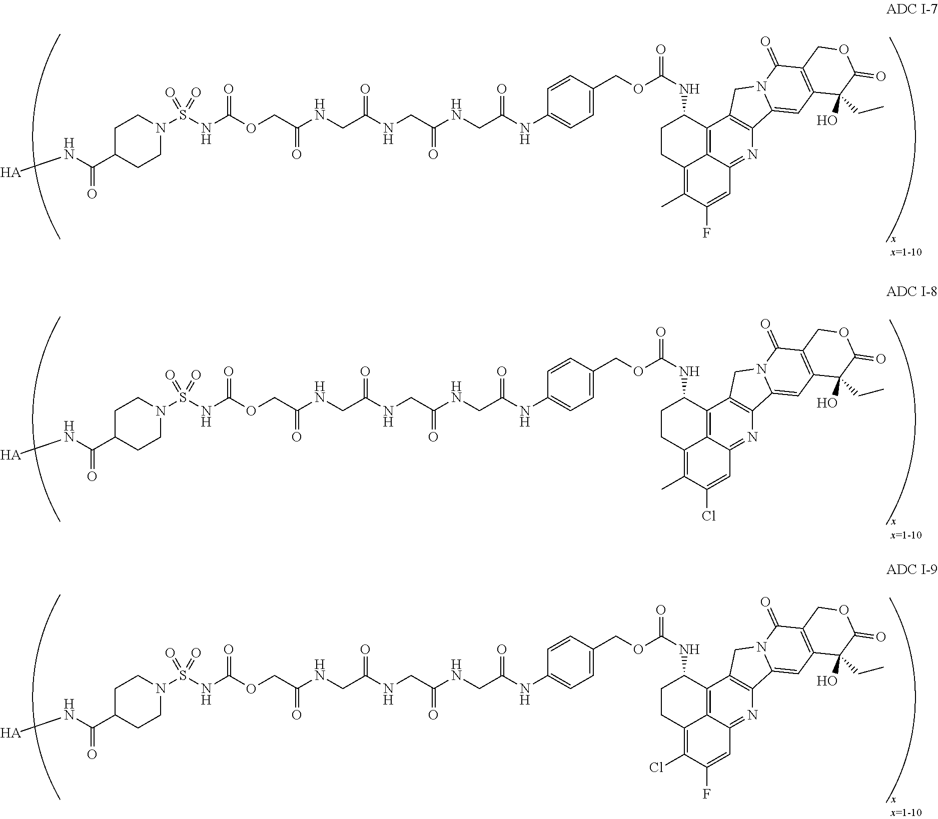

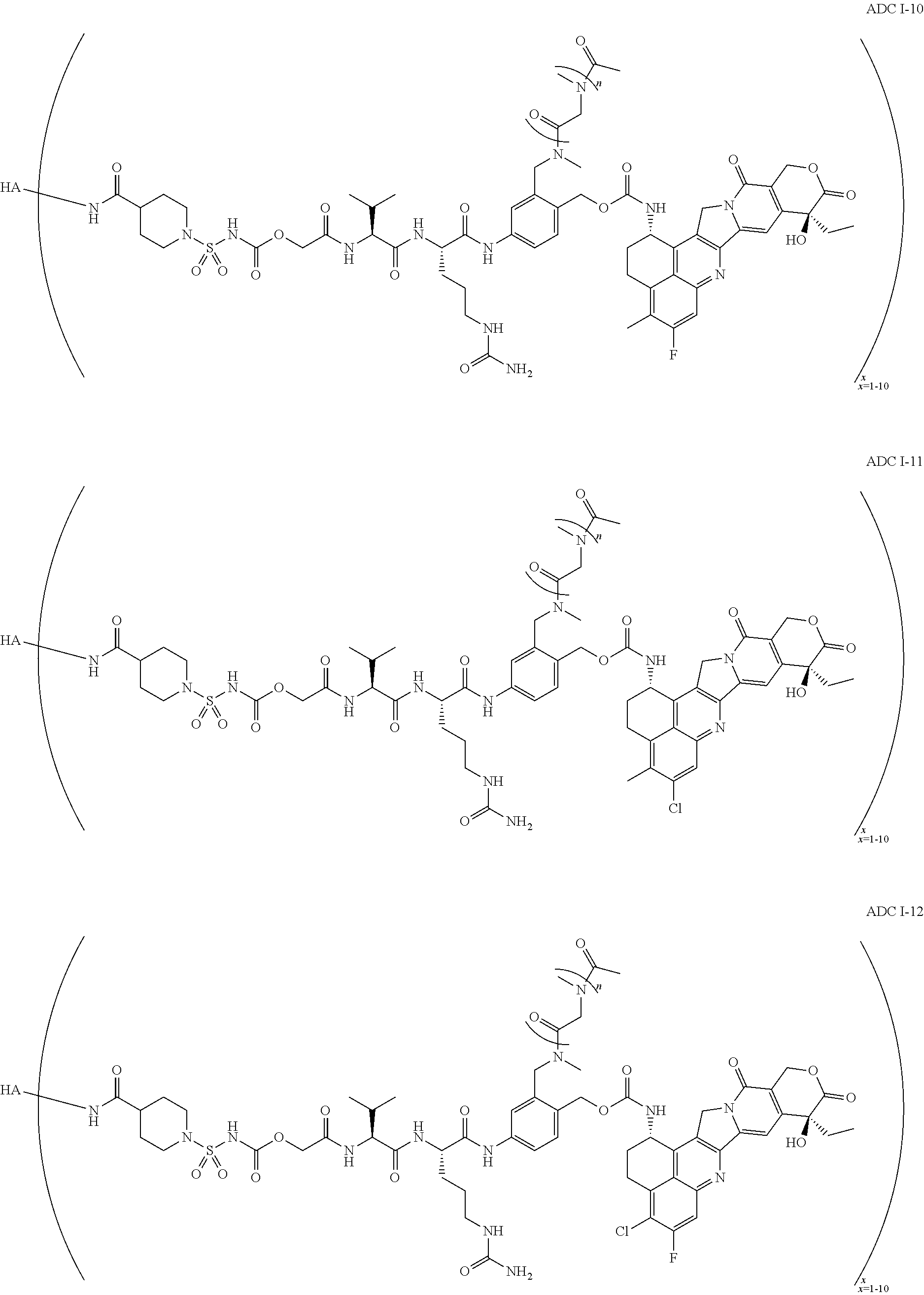

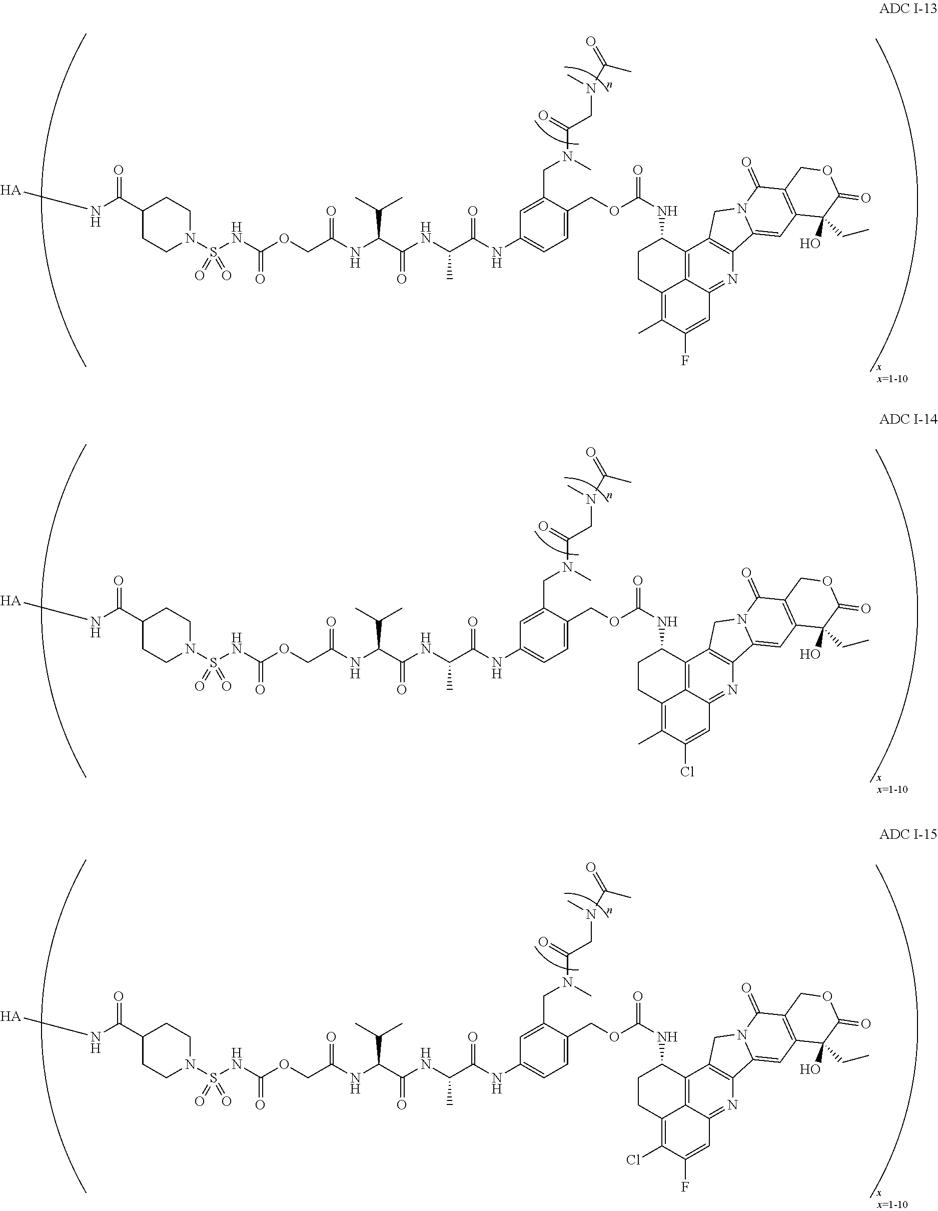

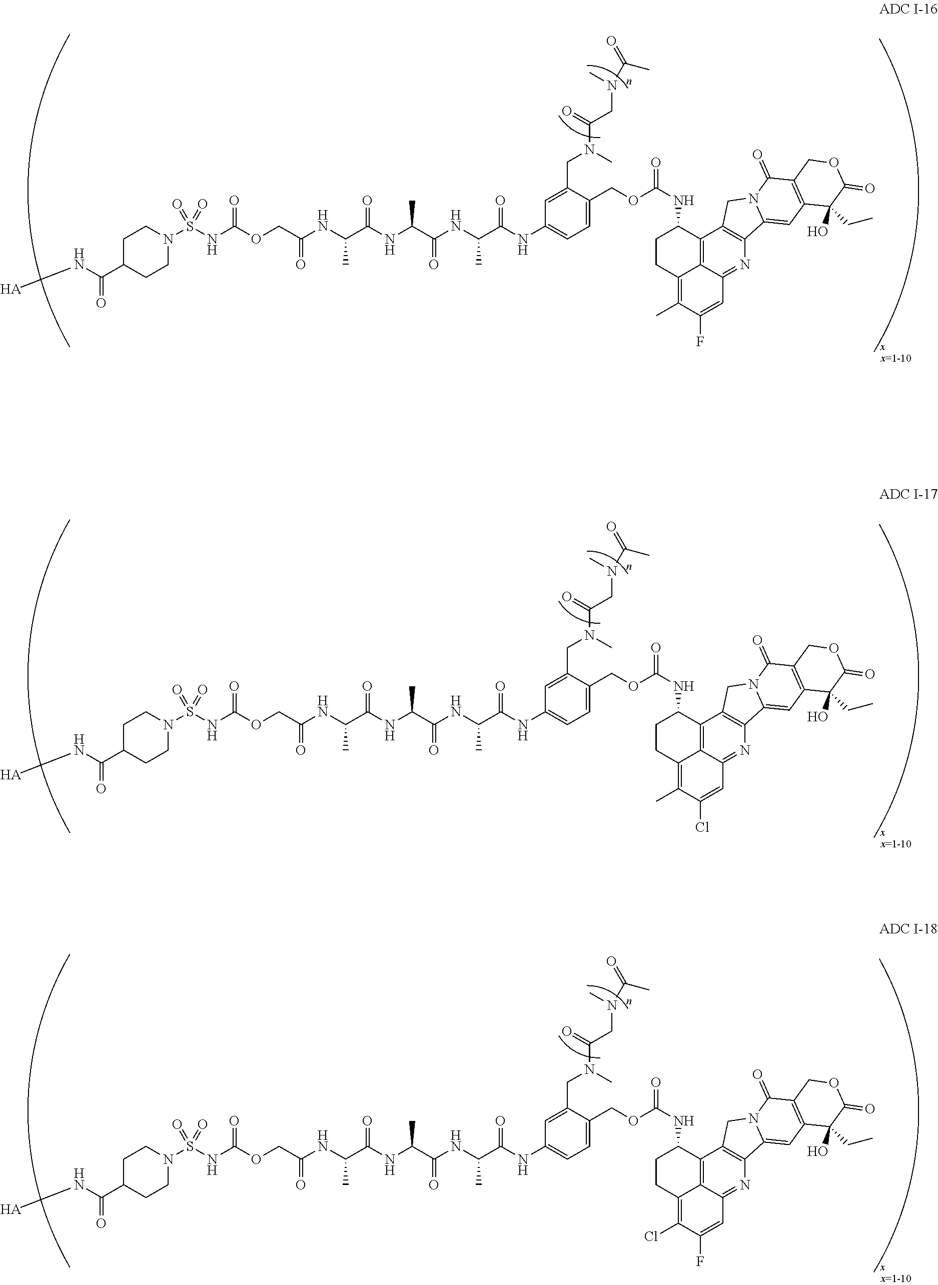

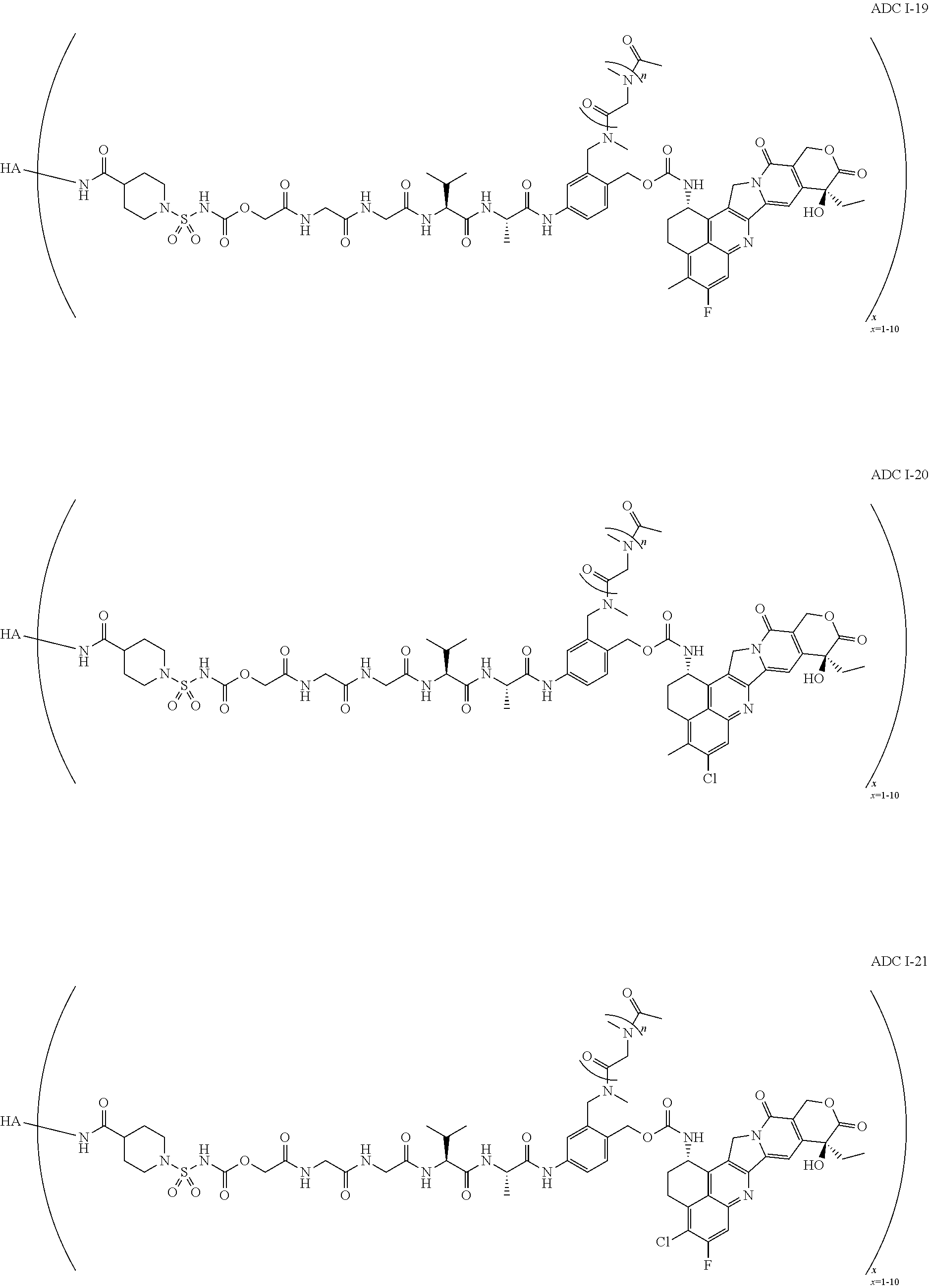

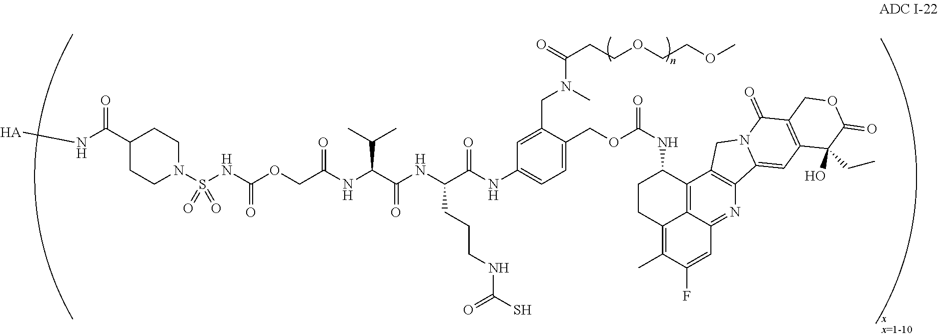

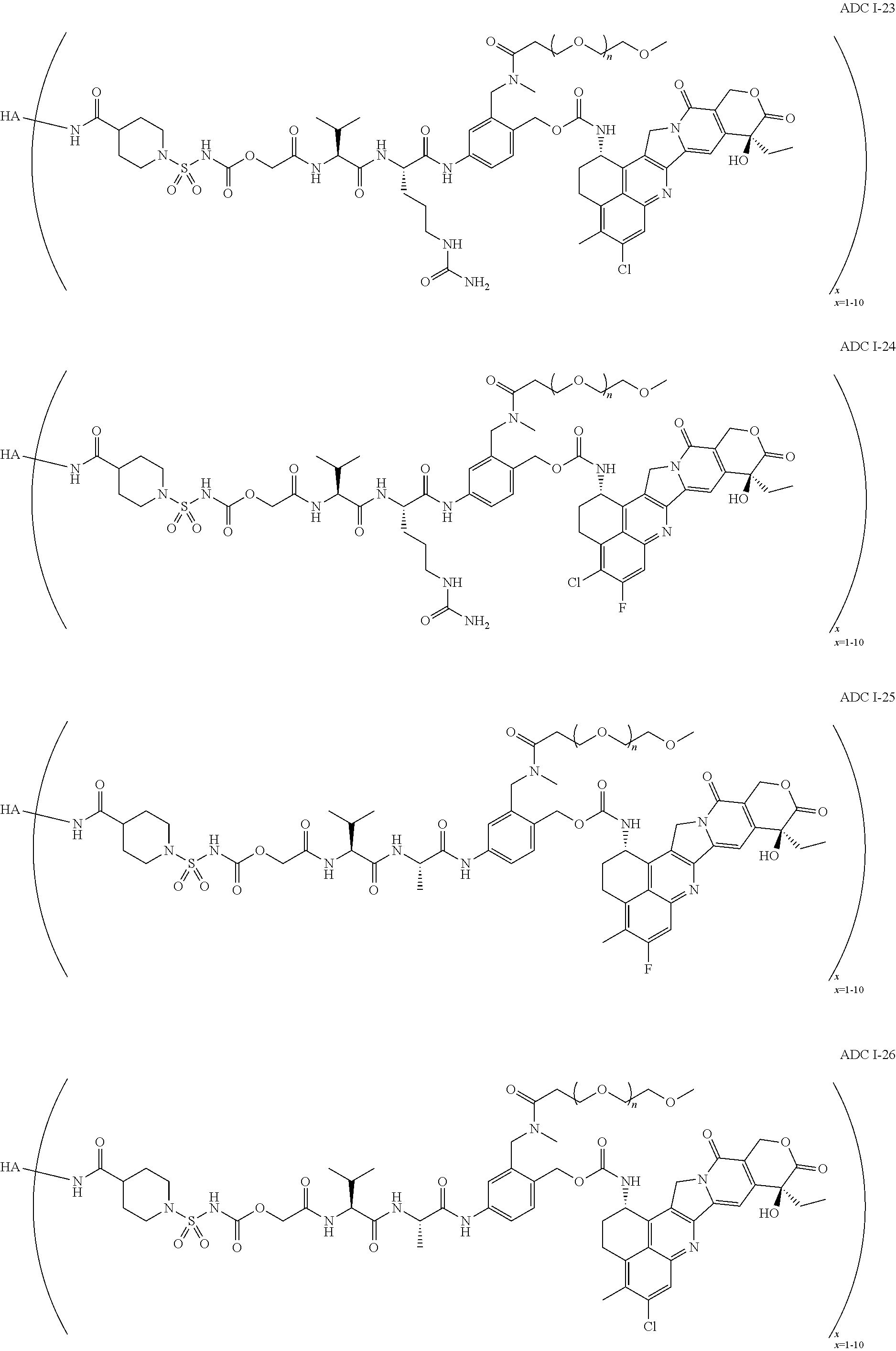

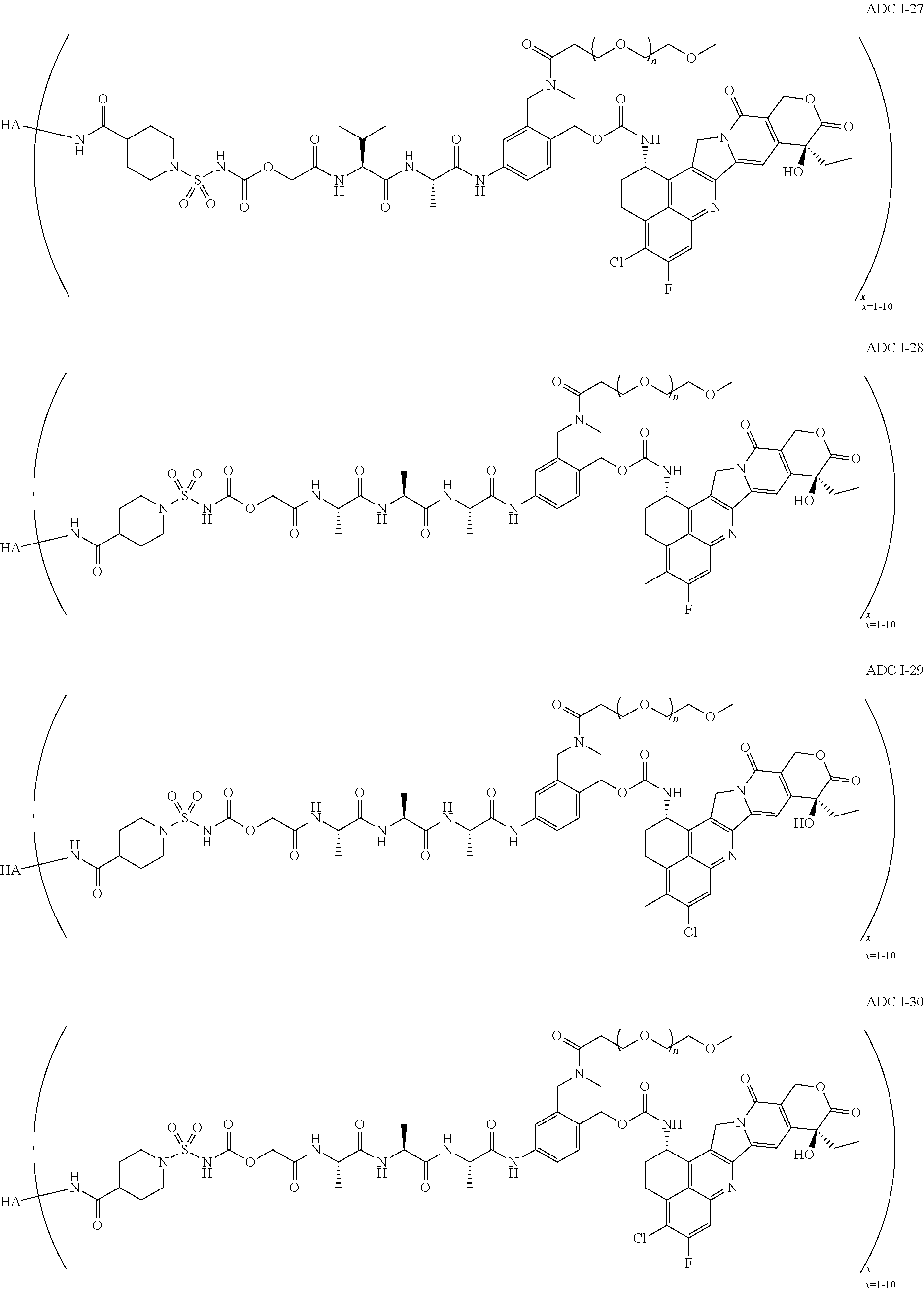

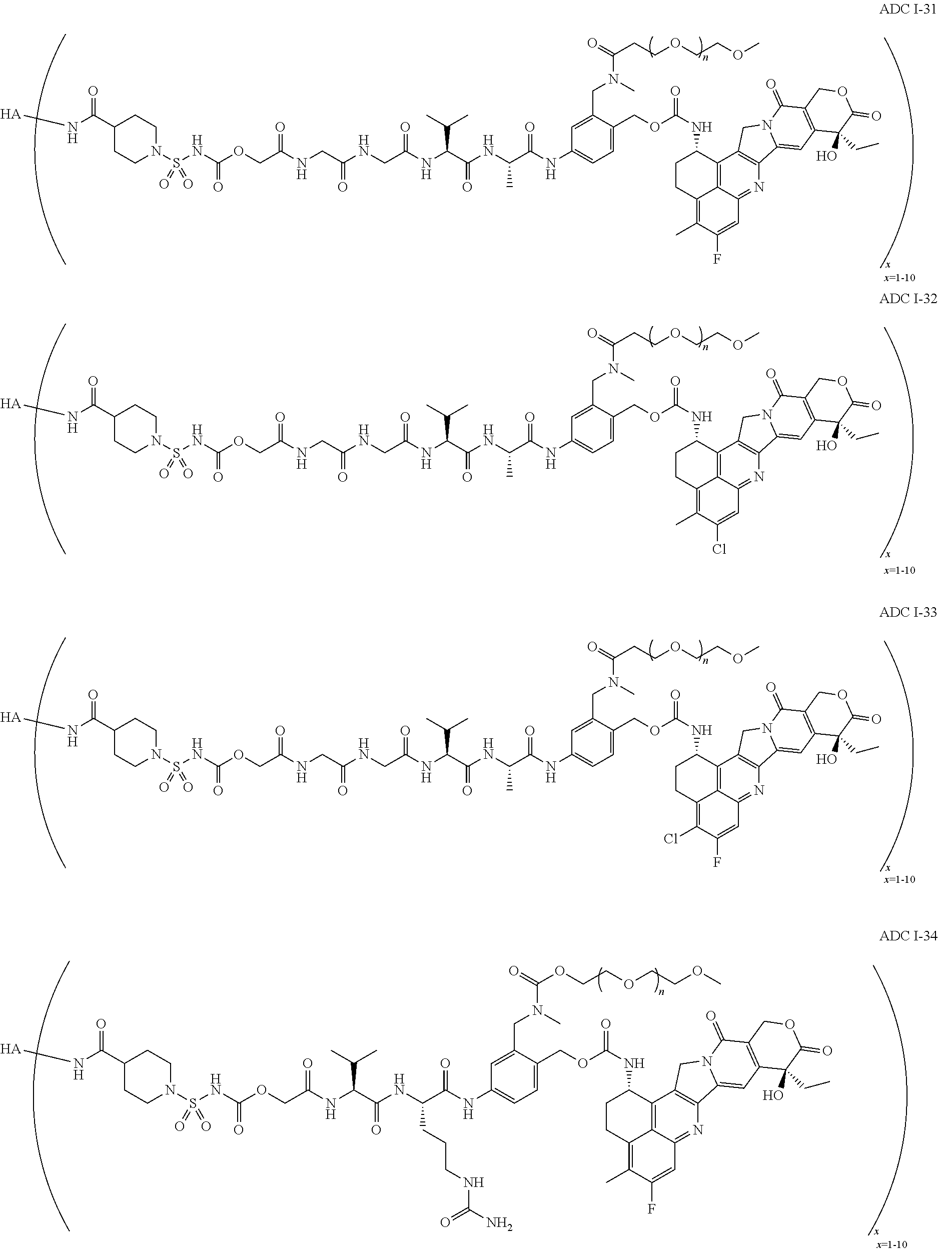

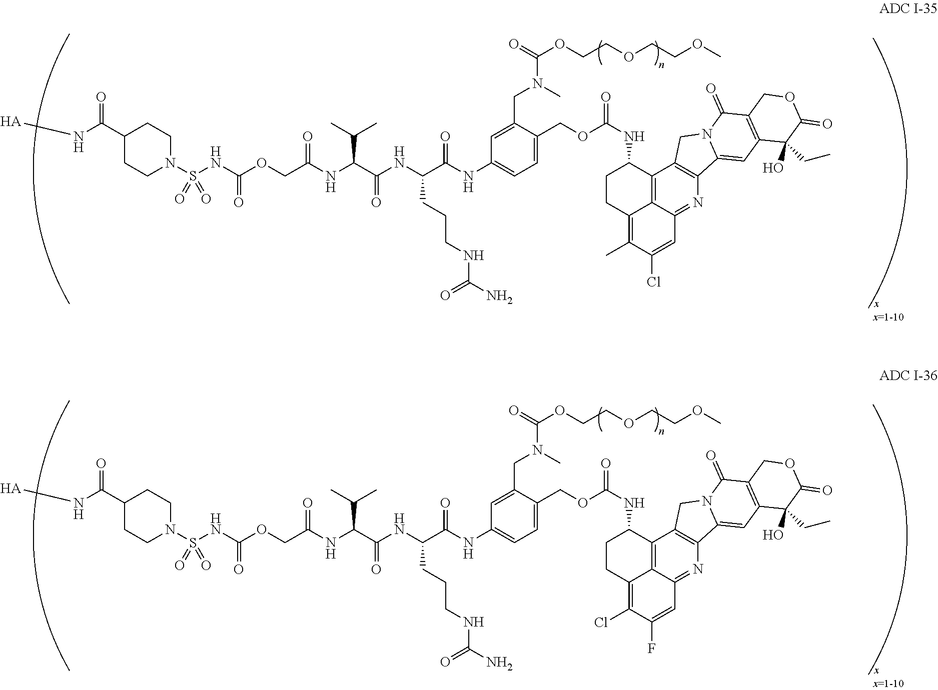

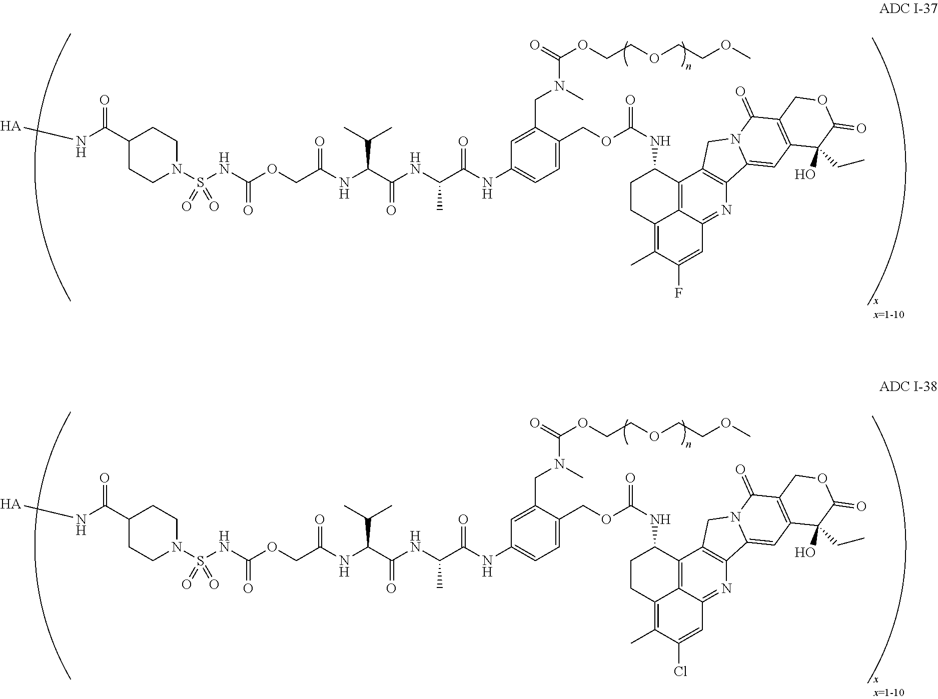

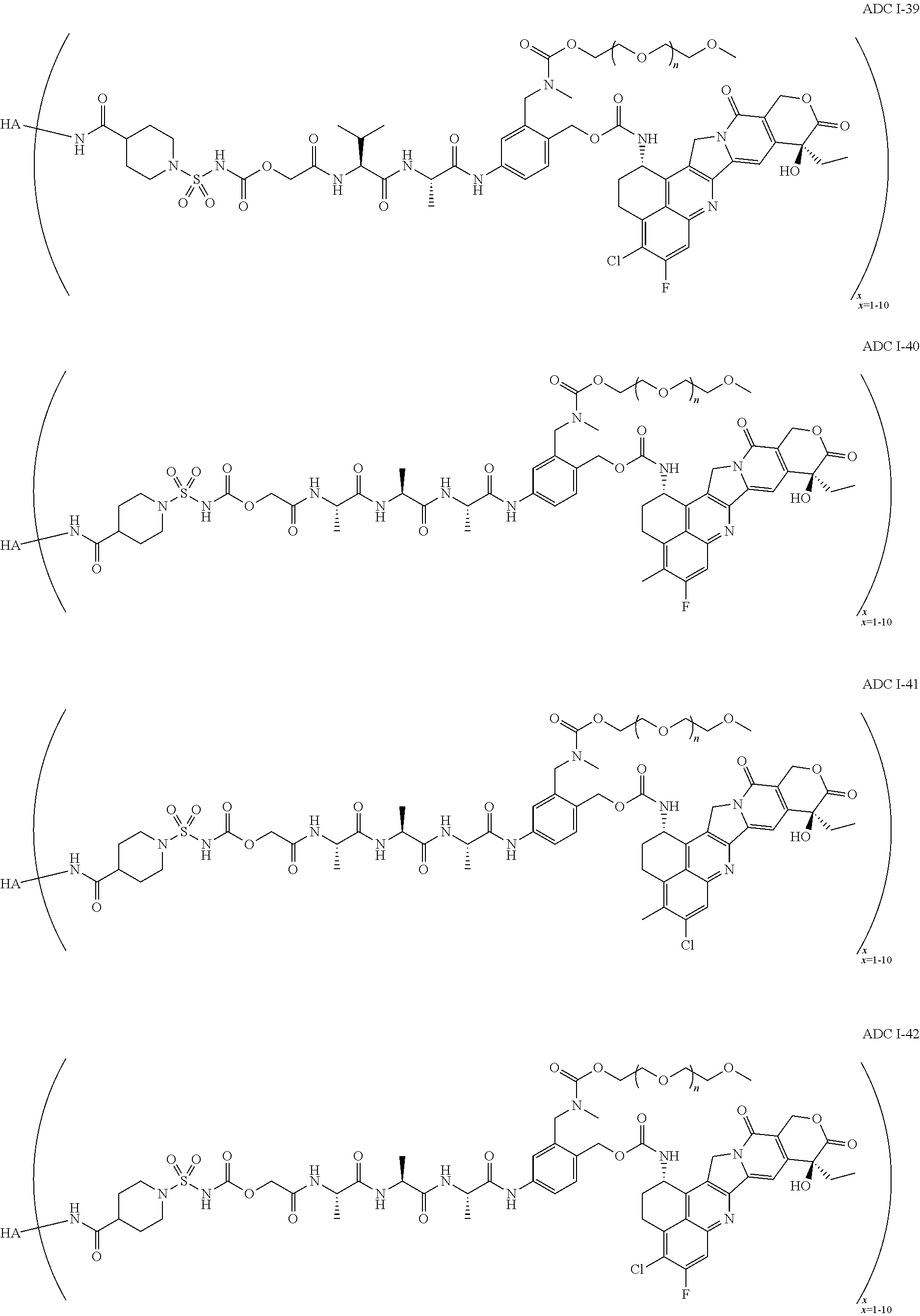

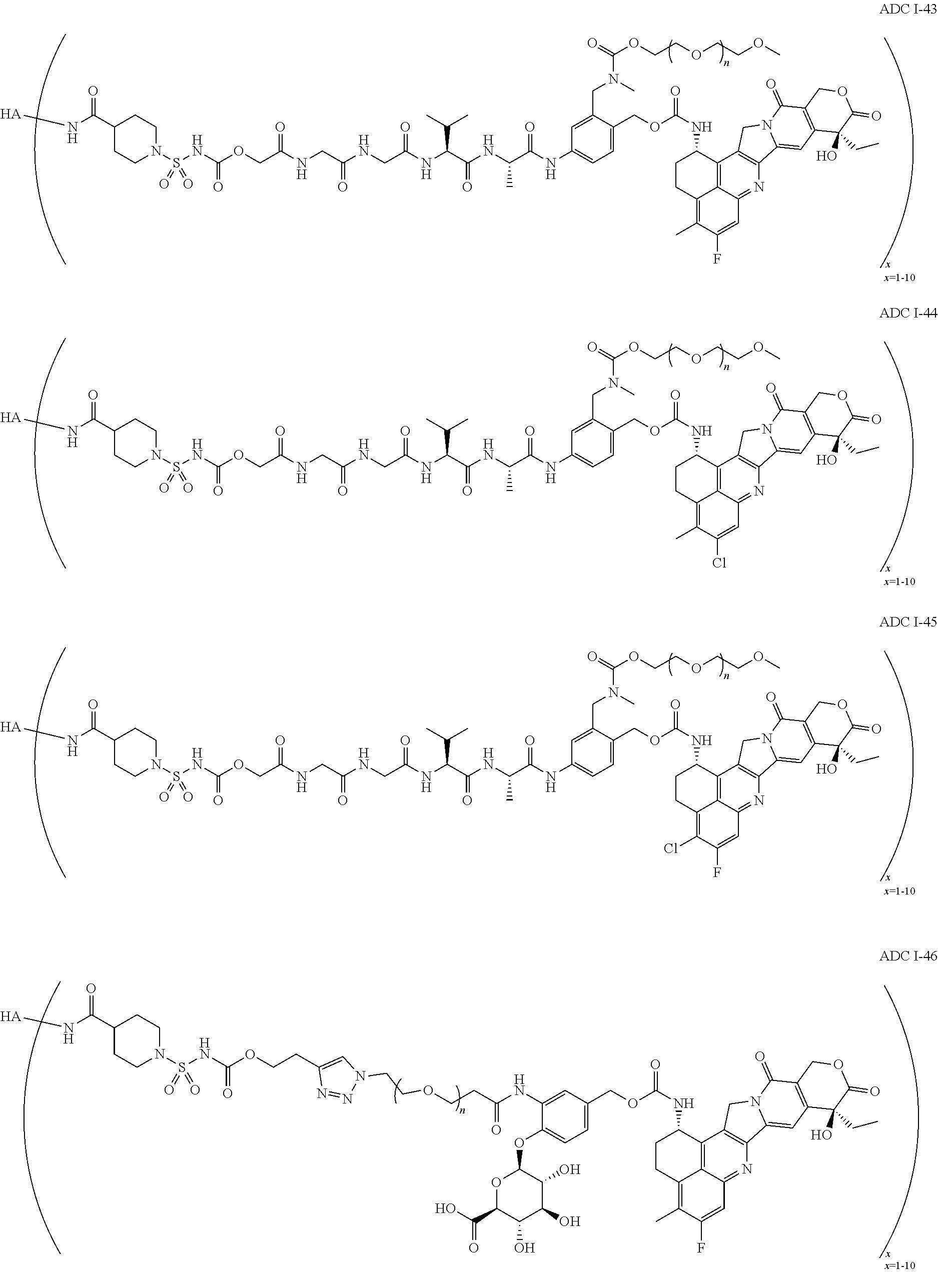

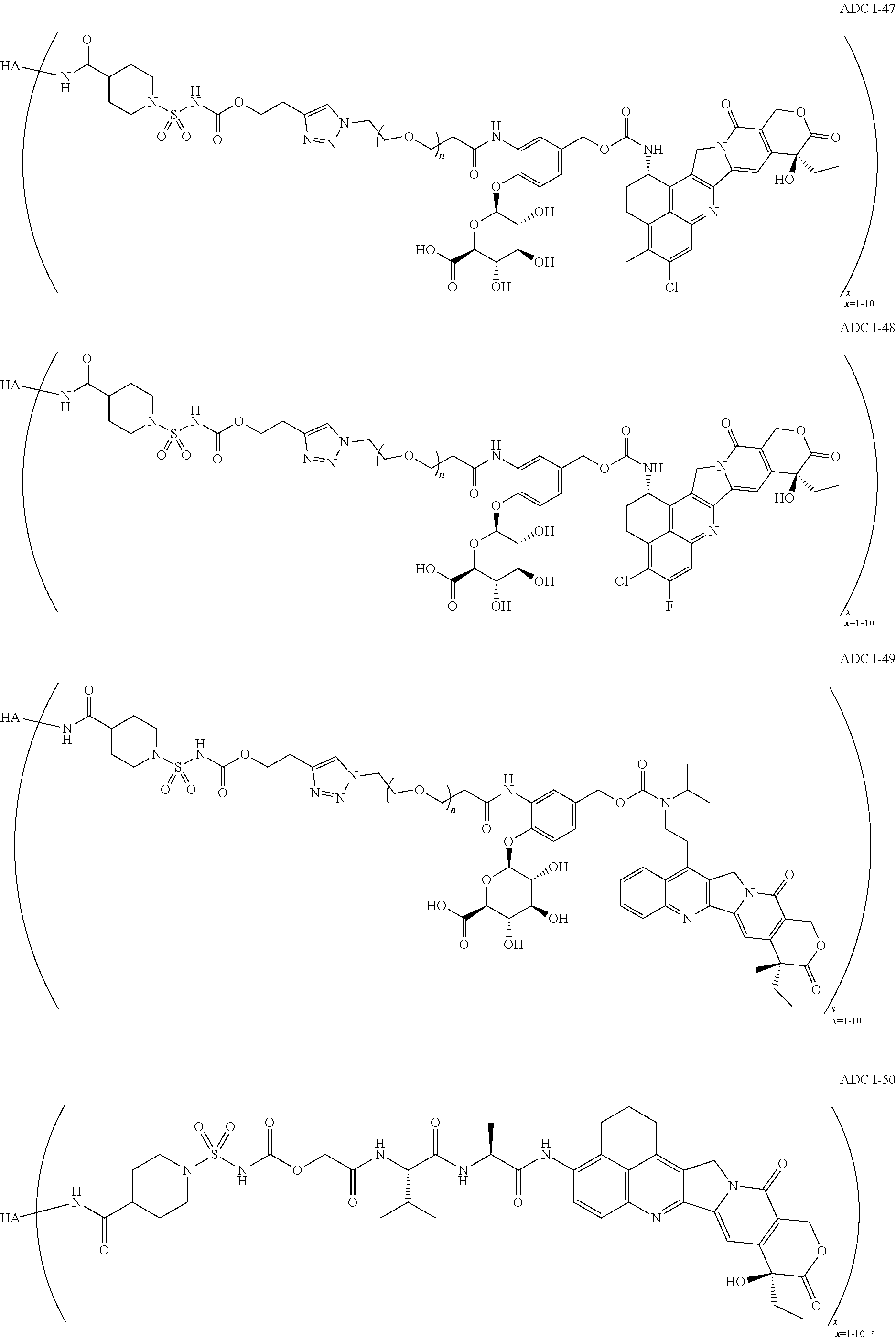

wherein, HA in each of the antibody-drug conjugate represents an antibody or antigen-binding fragment thereof capable of specifically binding to epidermal growth factor receptor 2 (Her2), a member of the ErbB family of receptor tyrosine kinases; n is an integer selected from 1 to 20, such as an integer of 1 to 12, 3 to 12, for example, n is 1, 2, 3, 4, 5, 6, 7, 8, 9, 10, 11, 12, 13, 14 or 15;
wherein,

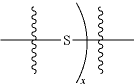 [SMILES: *S*]

represents a specific linkage between a sulfhydryl group in the antibody or antigen-binding fragment thereof and the M fragment;

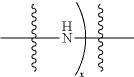 [SMILES: *N*]

represents a specific linkage between an amino group in the antibody or antigen-binding fragment thereof and the M fragment.

In some embodiments, the antibody-drug conjugate is selected from the group consisting of ADC E′-1 to ADC E′-50, ADC F′-1 to ADC F′-3, ADC F′-7 to ADC F′-12, ADC G′-1 to G′-4, ADC I′-10 to I′-49 as shown in below:

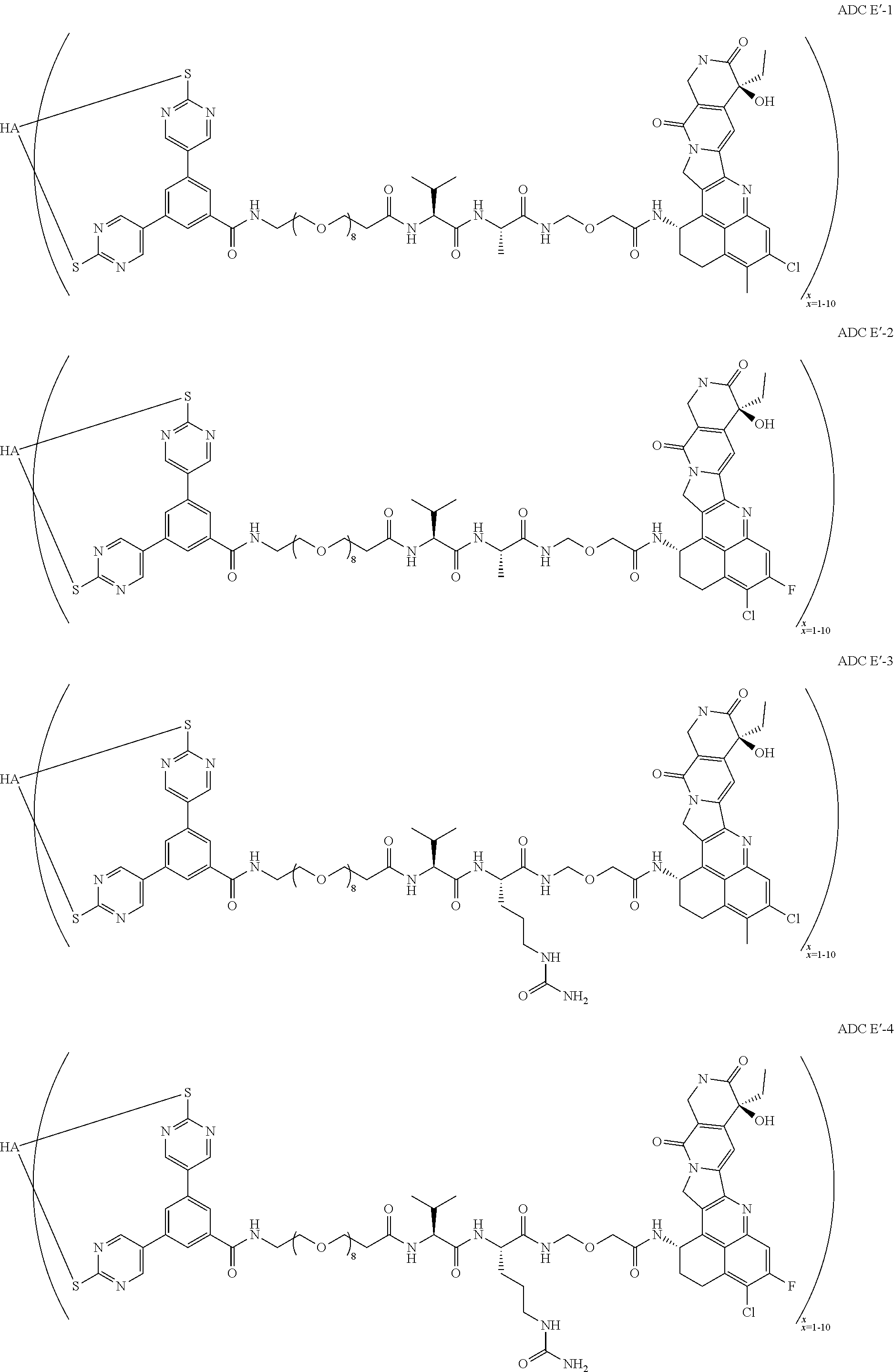

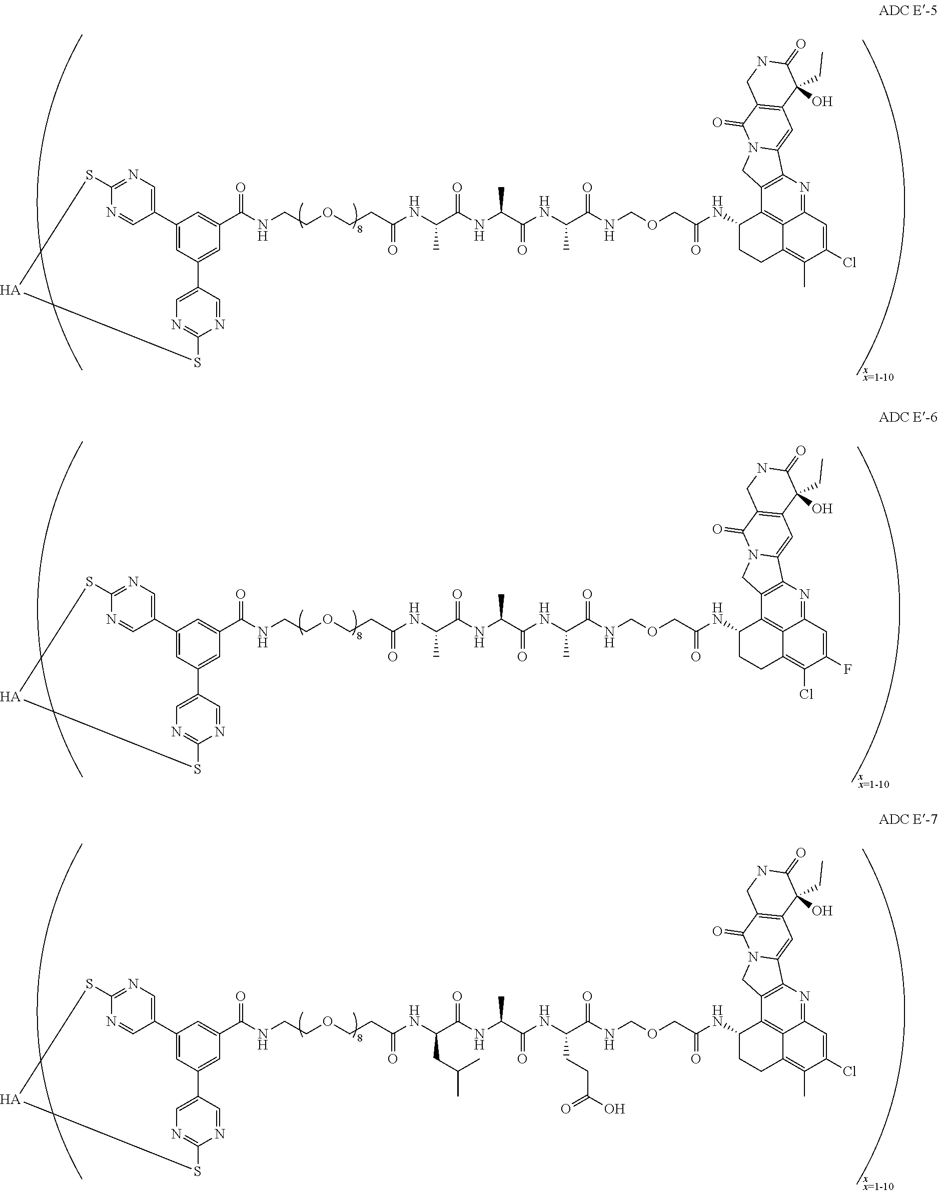

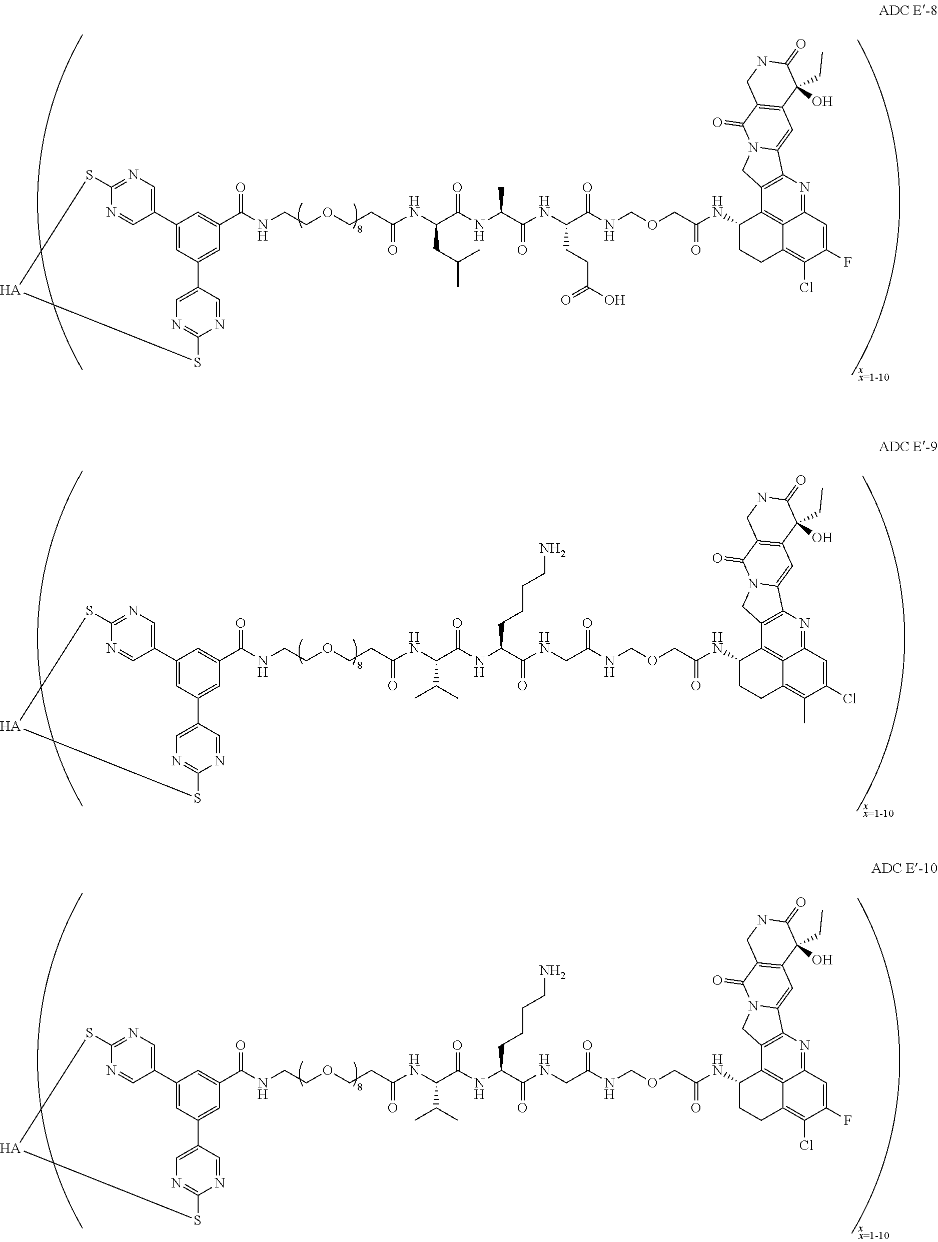

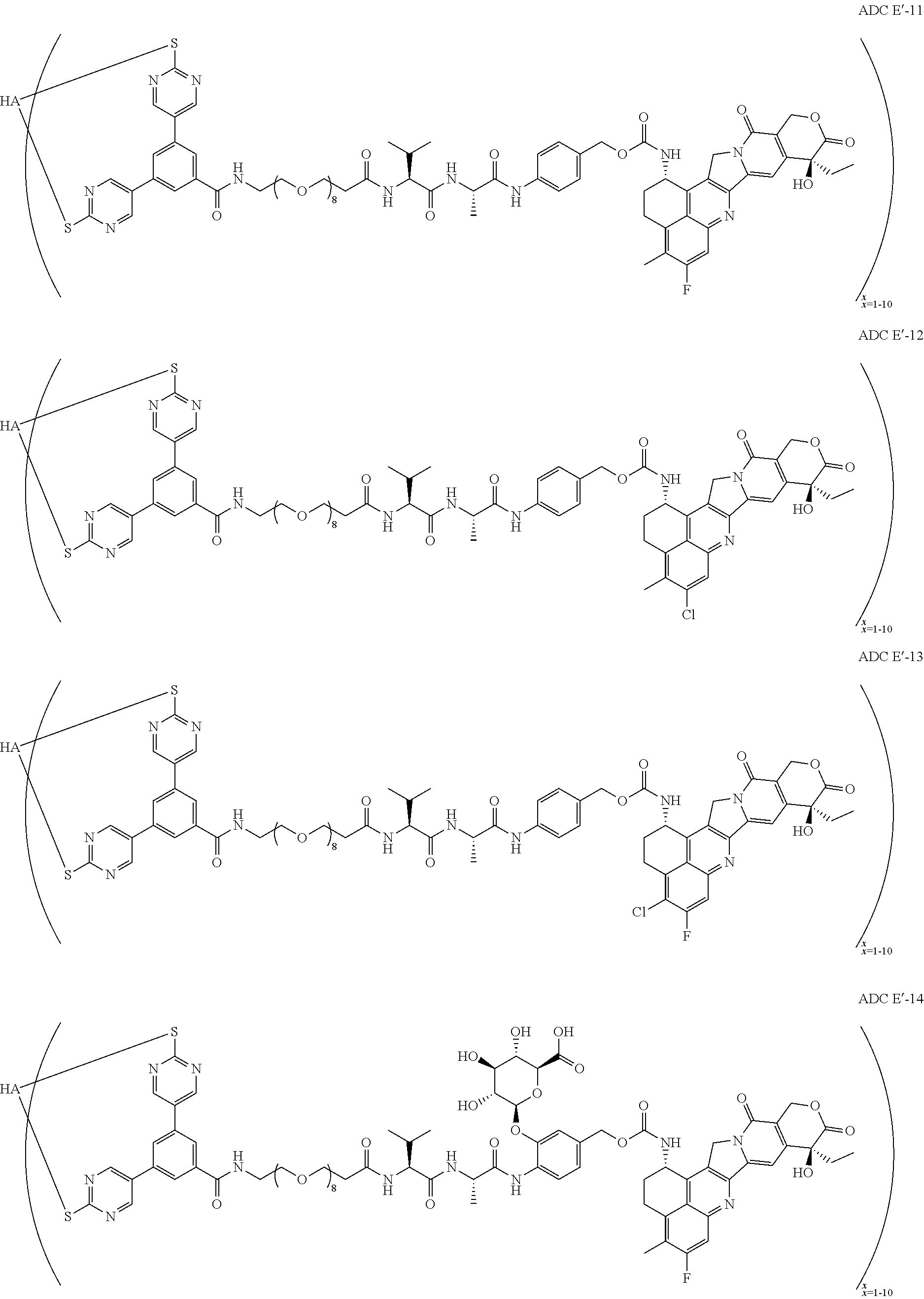

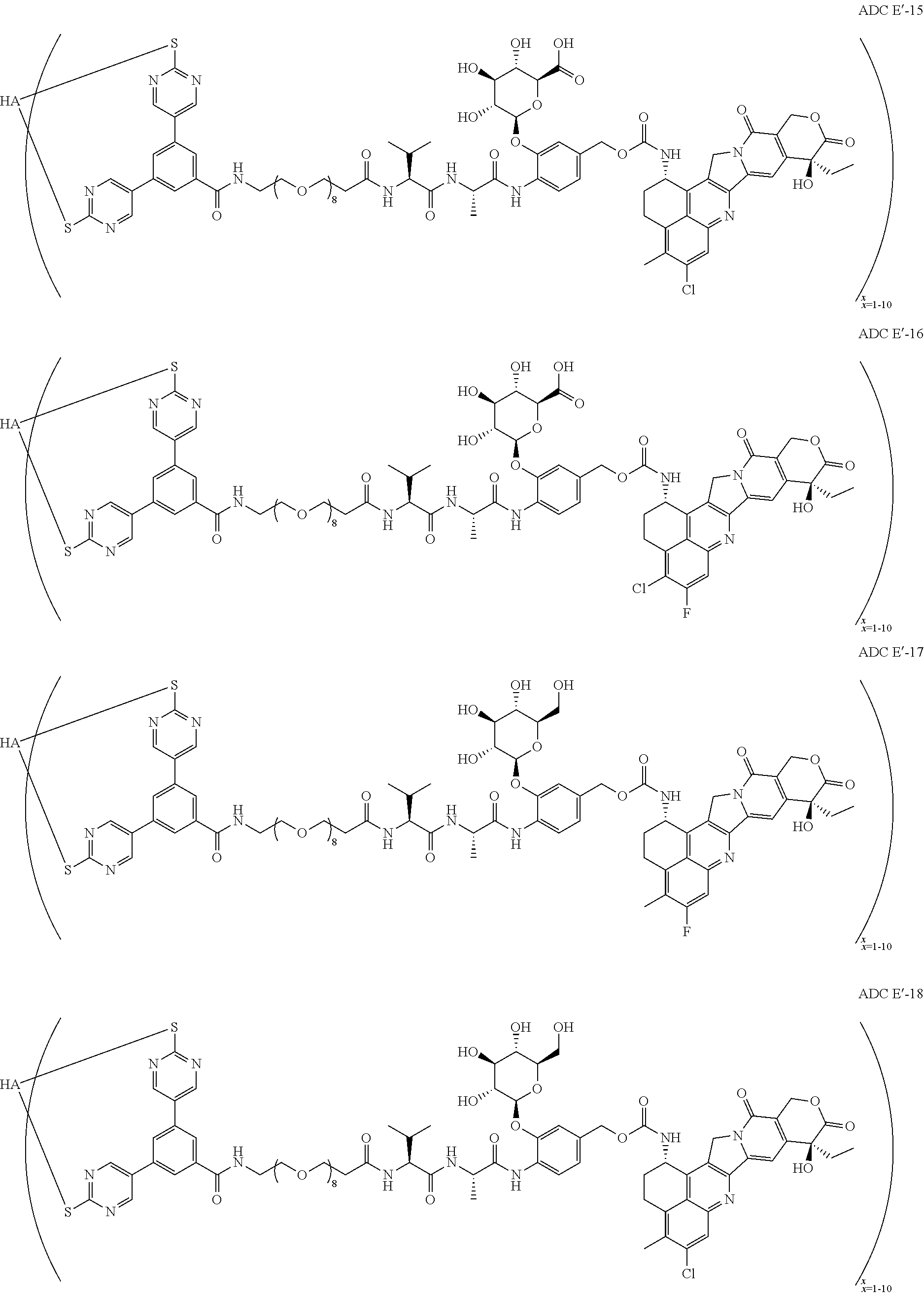

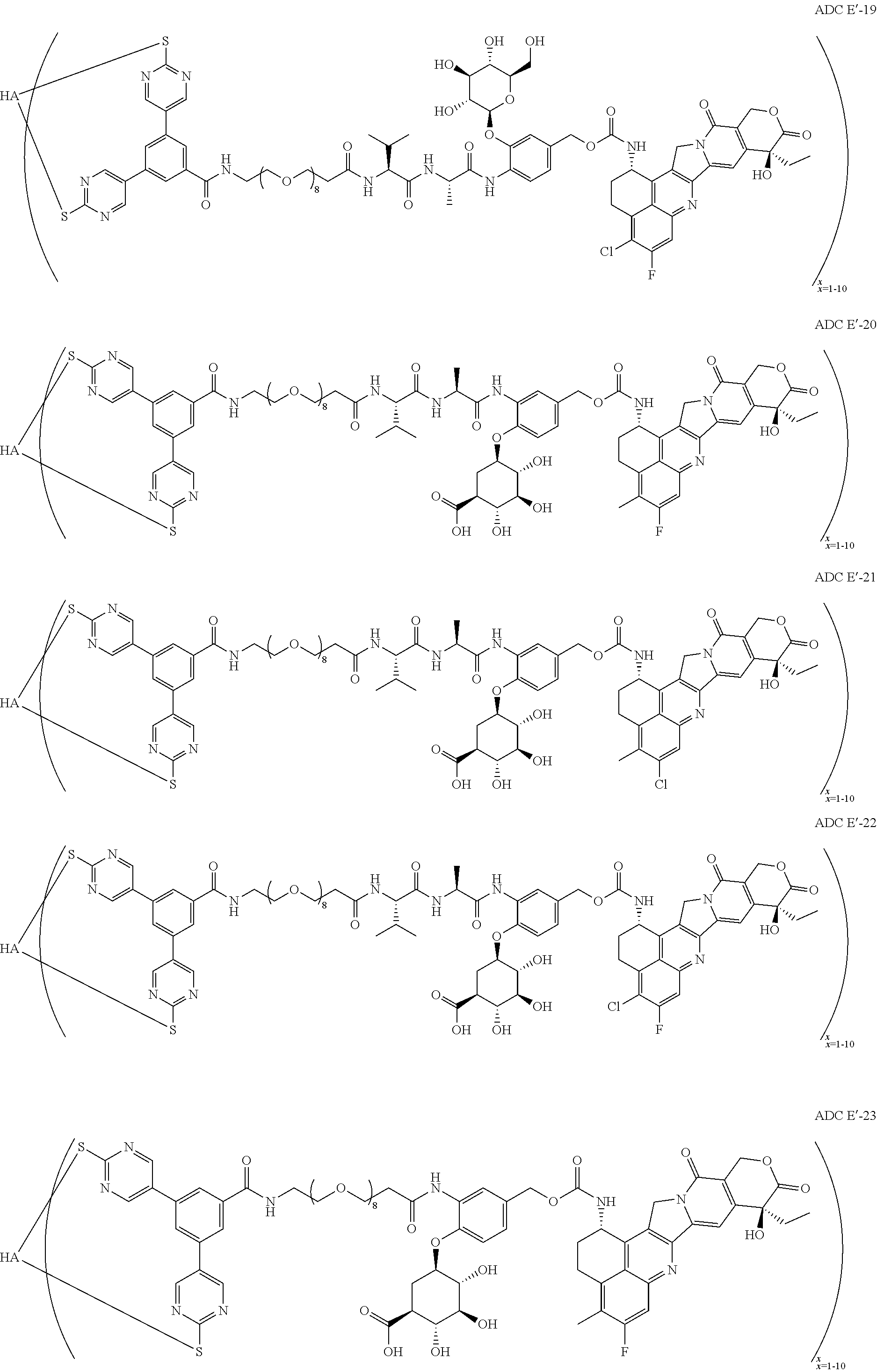

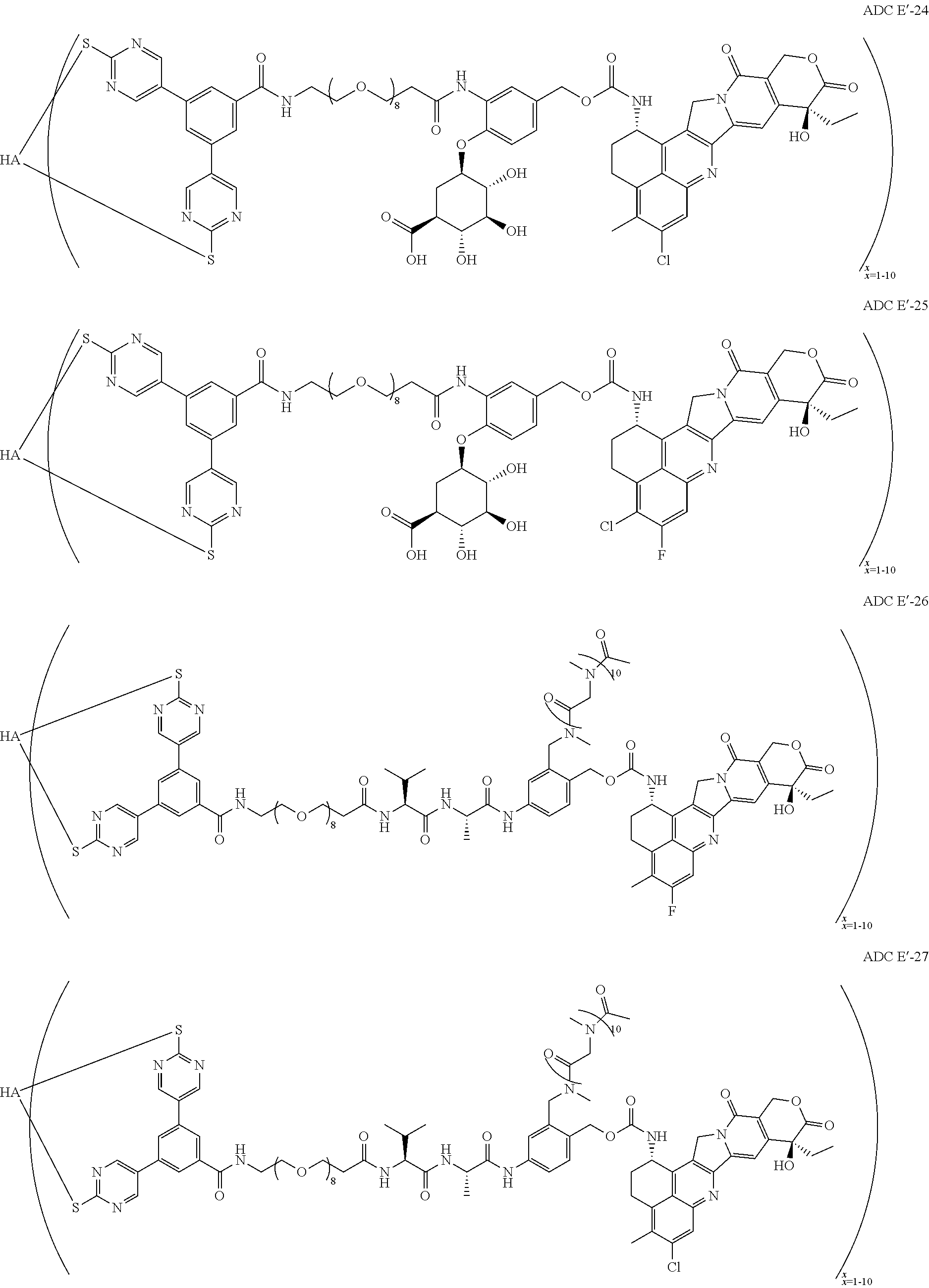

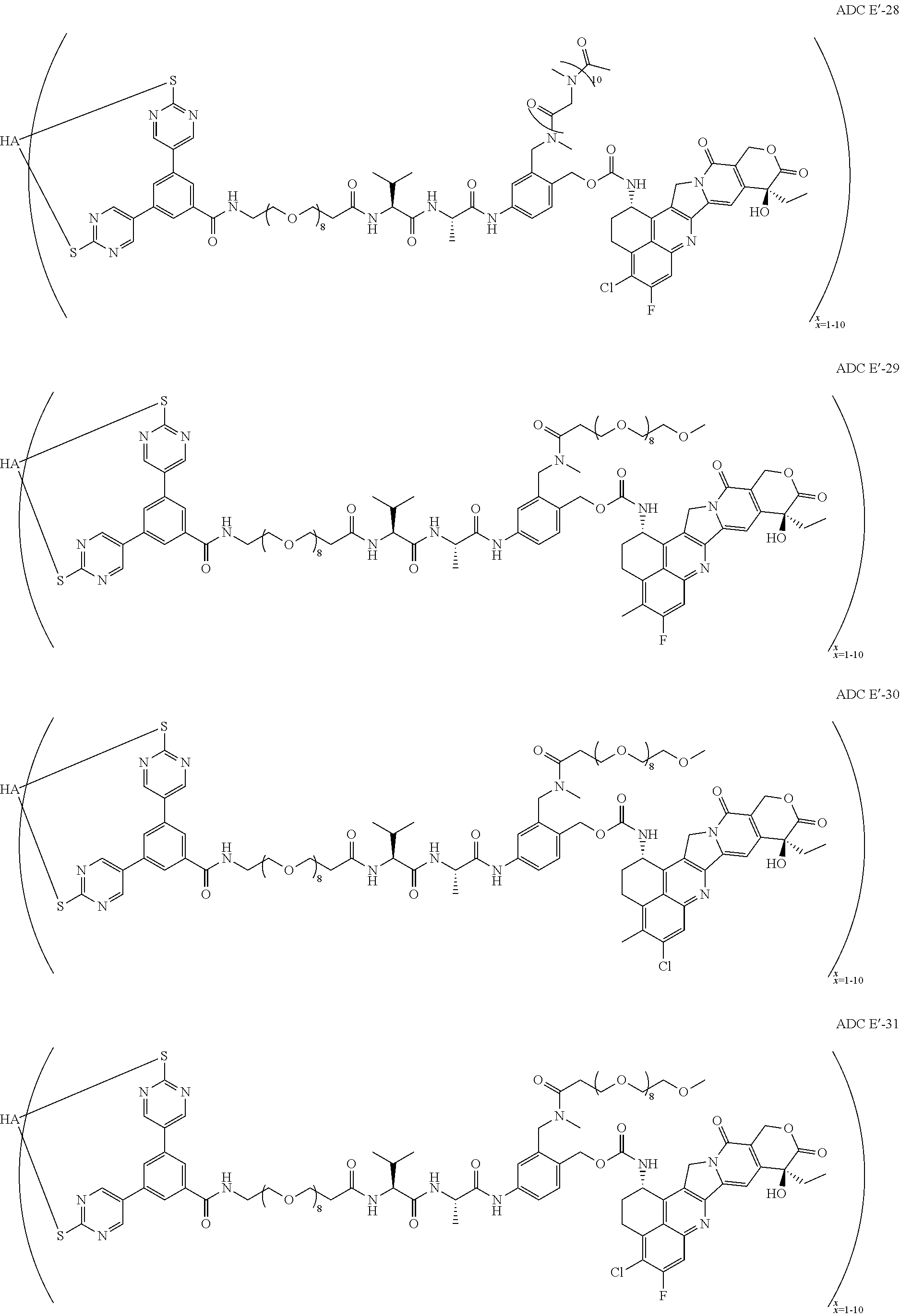

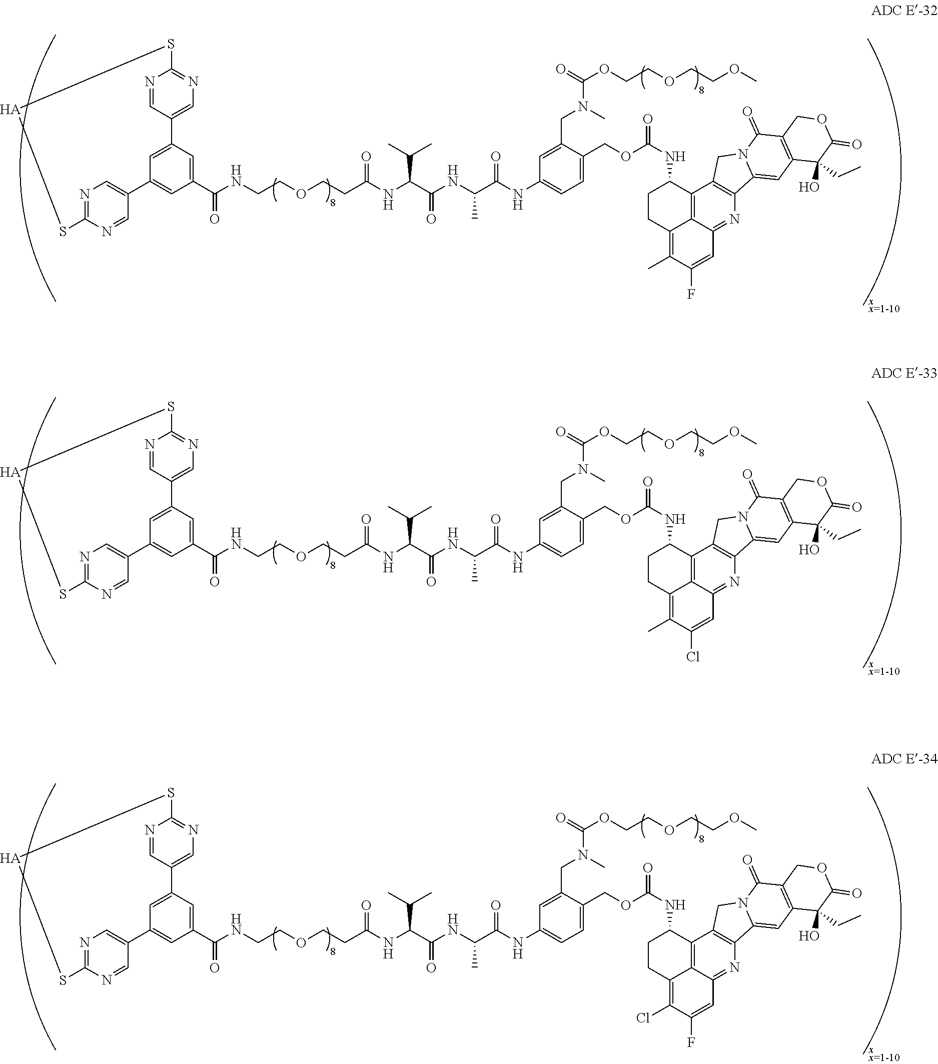

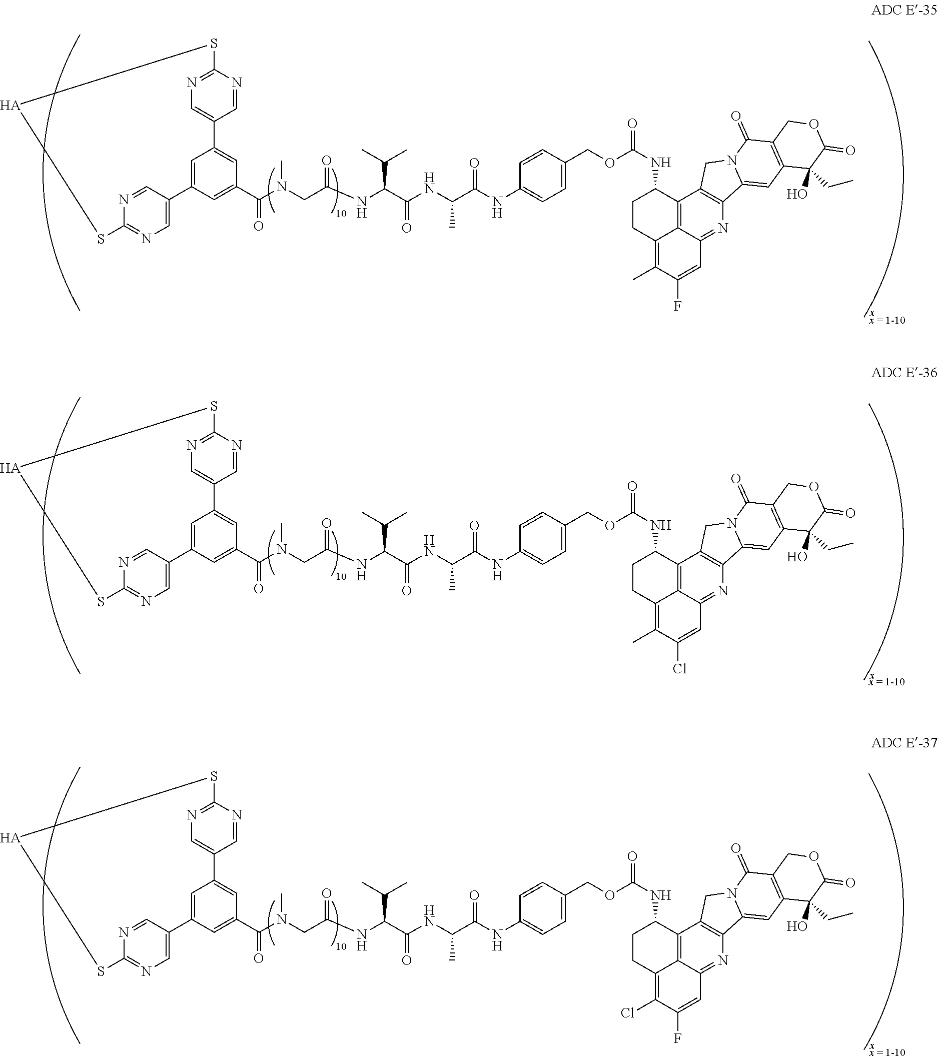

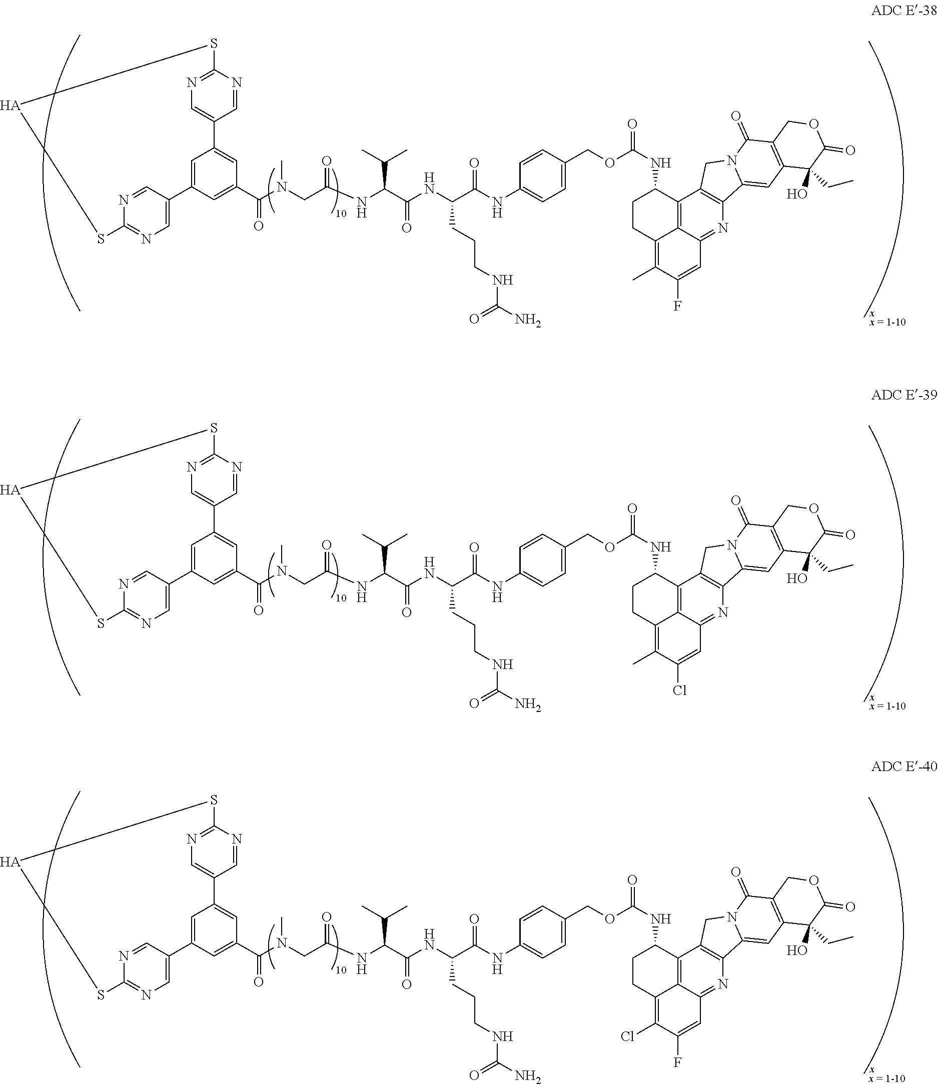

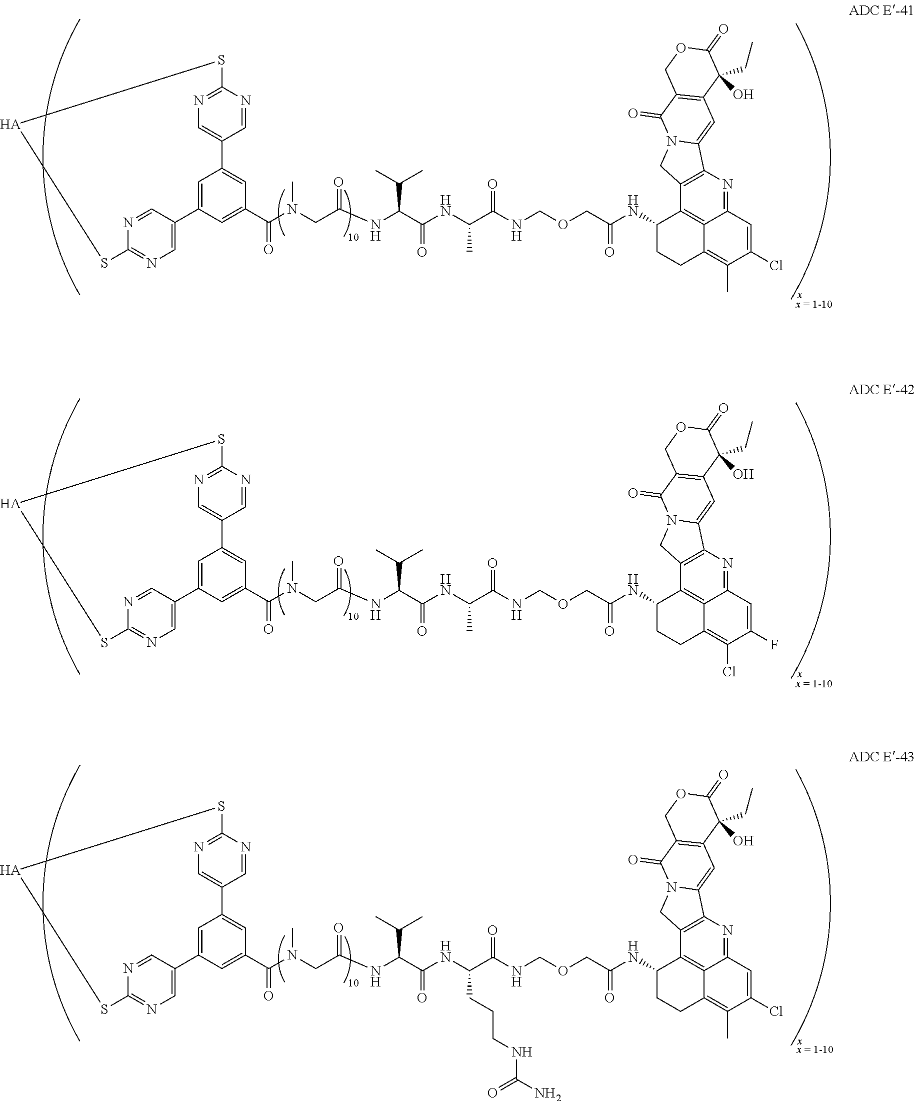

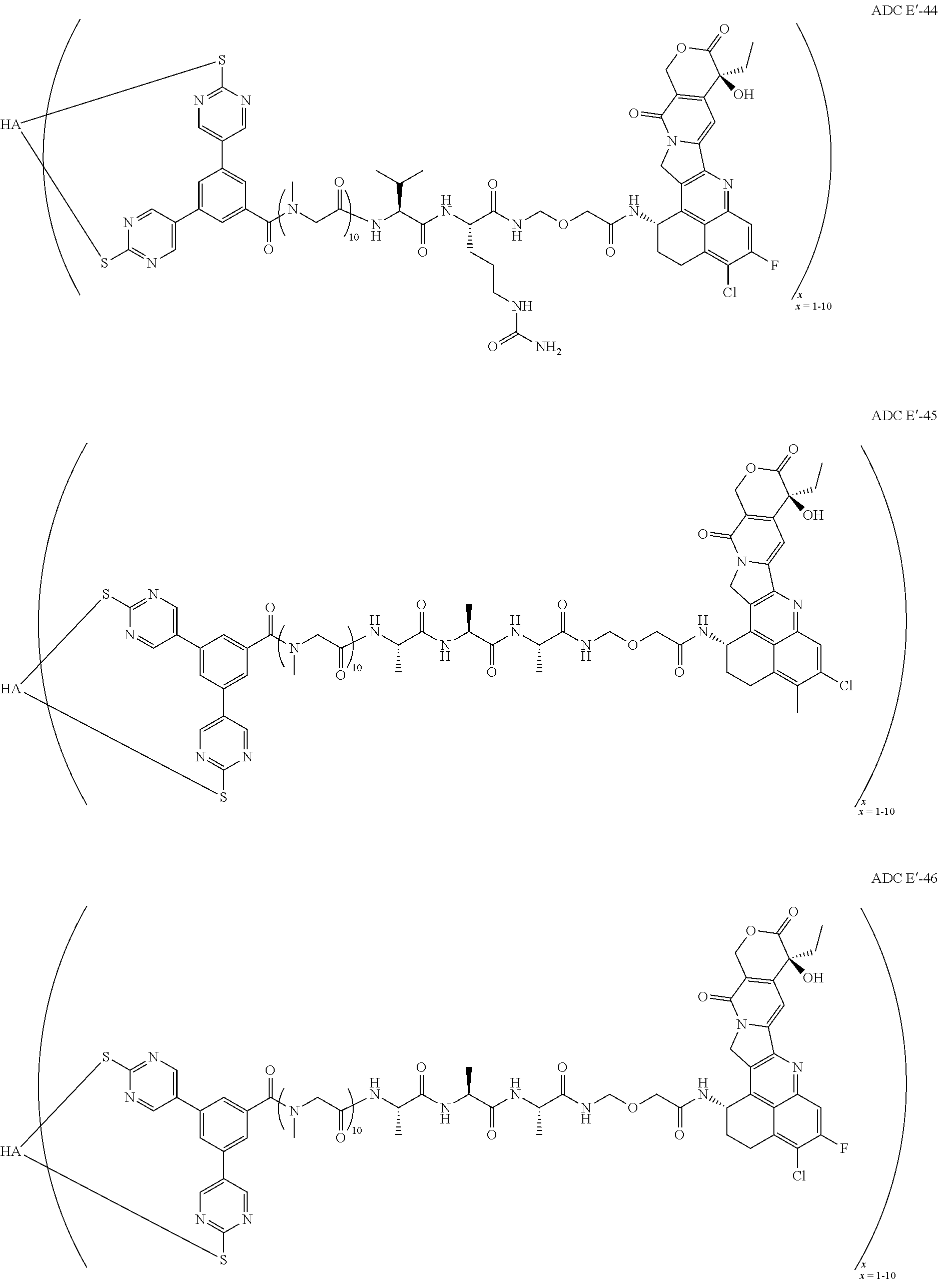

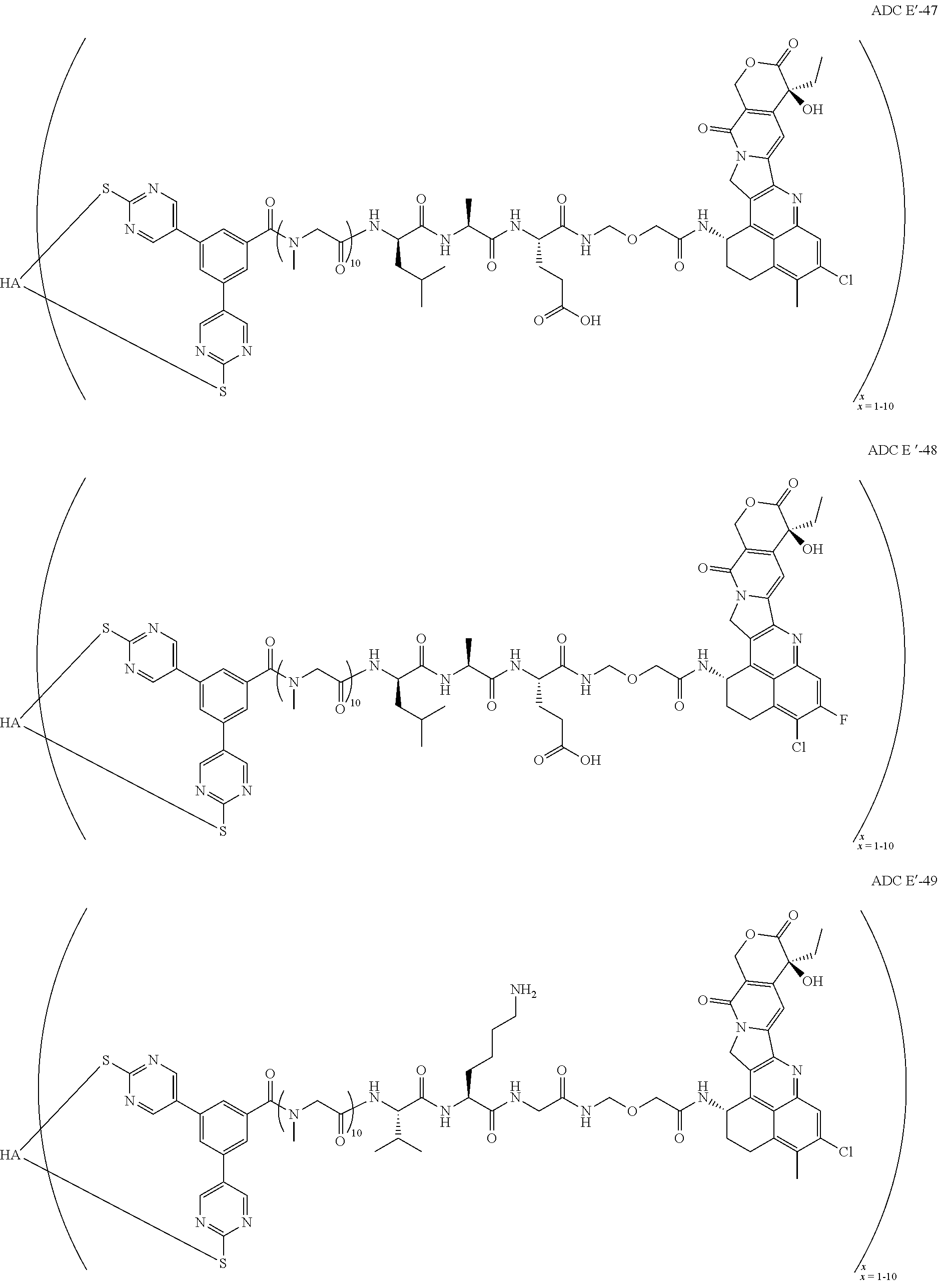

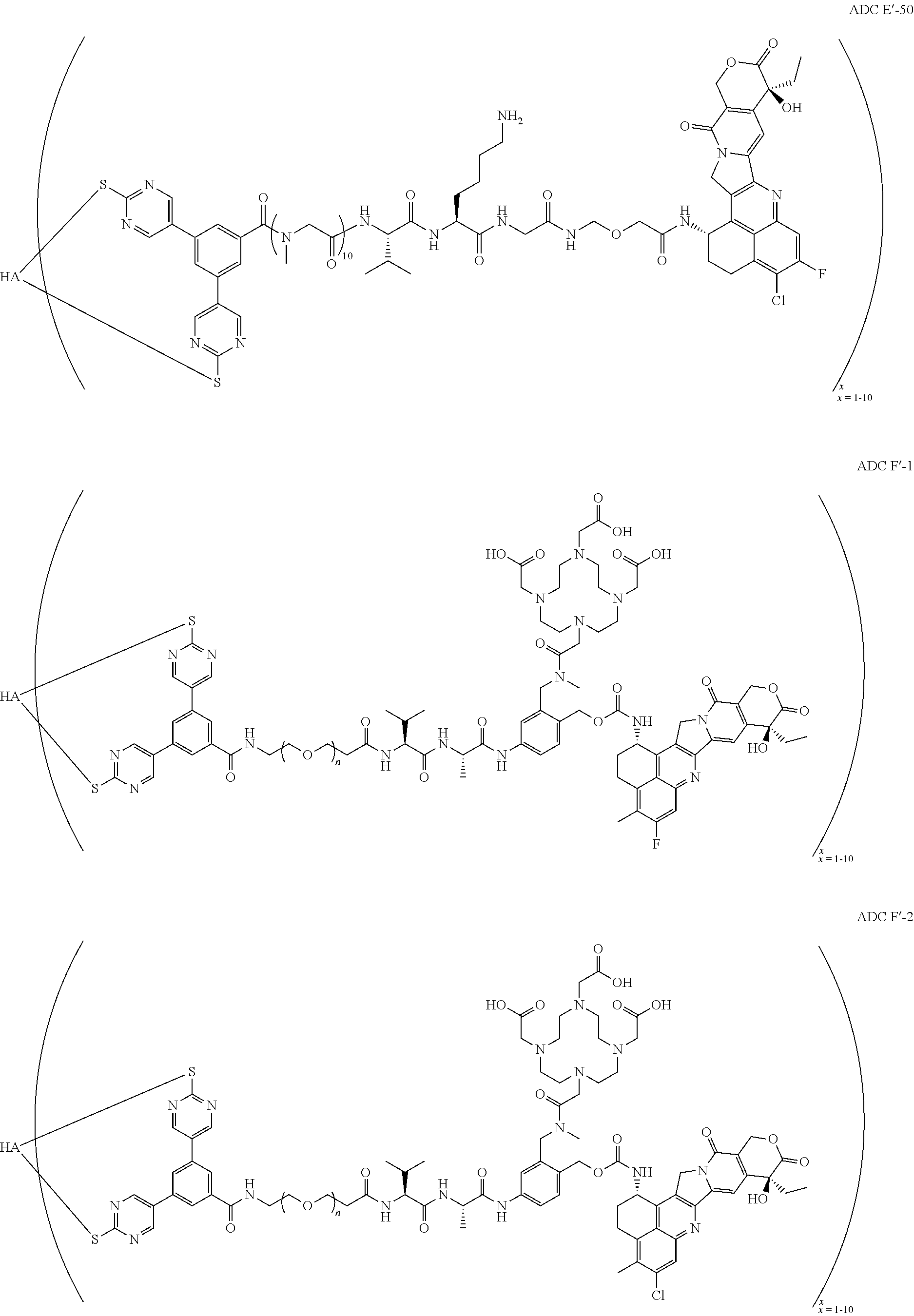

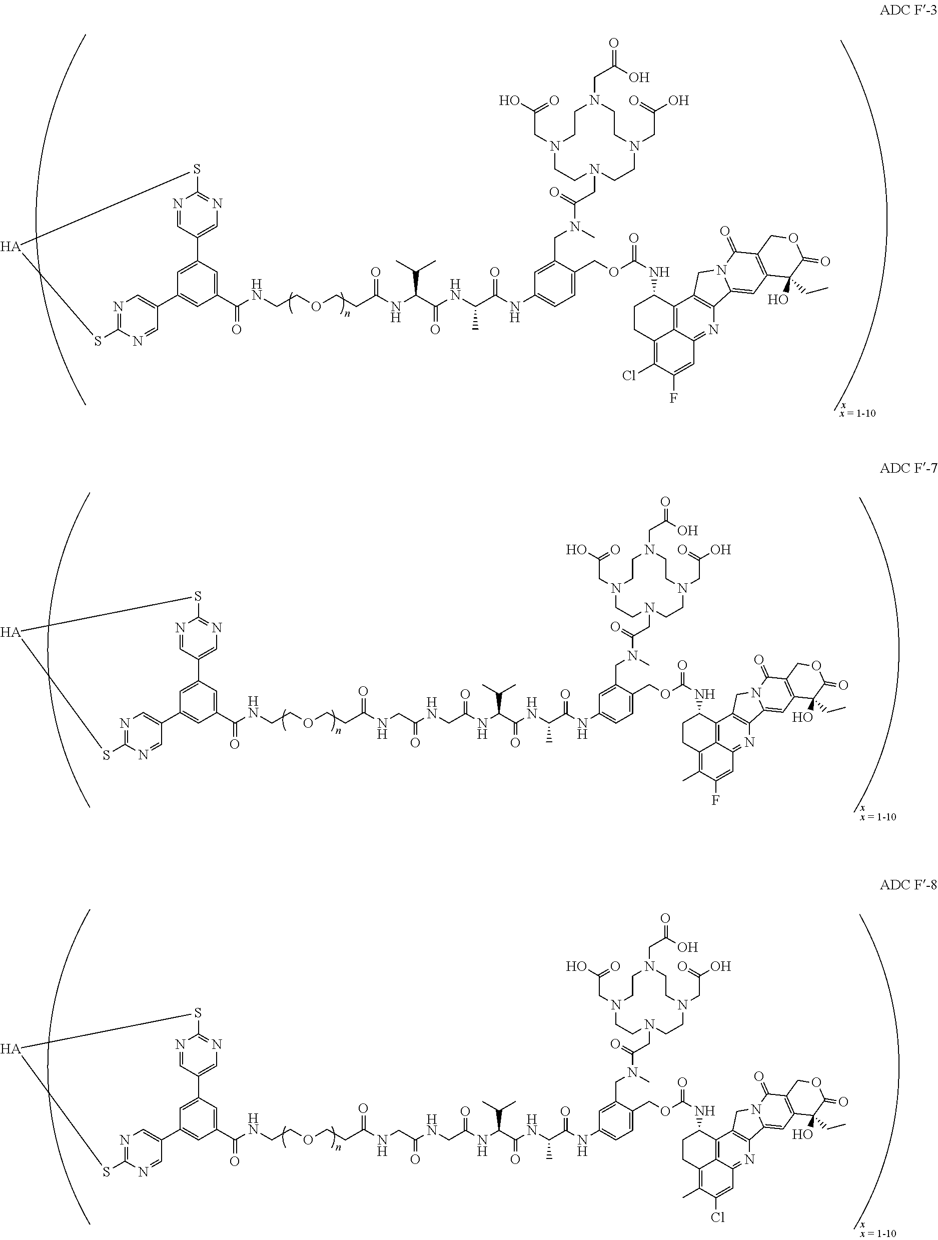

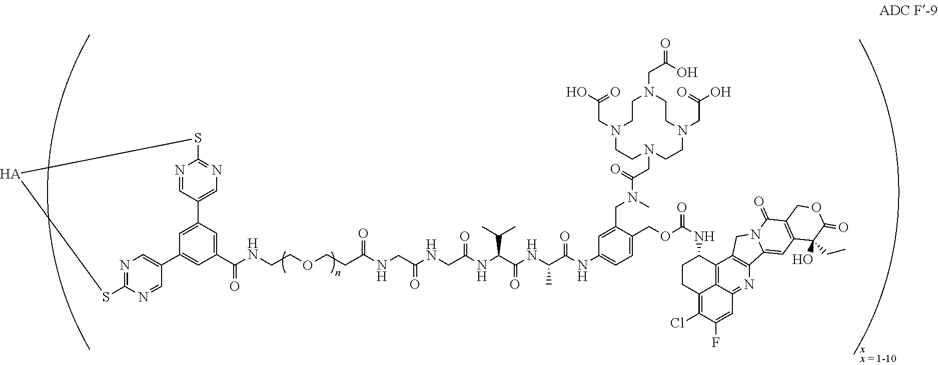

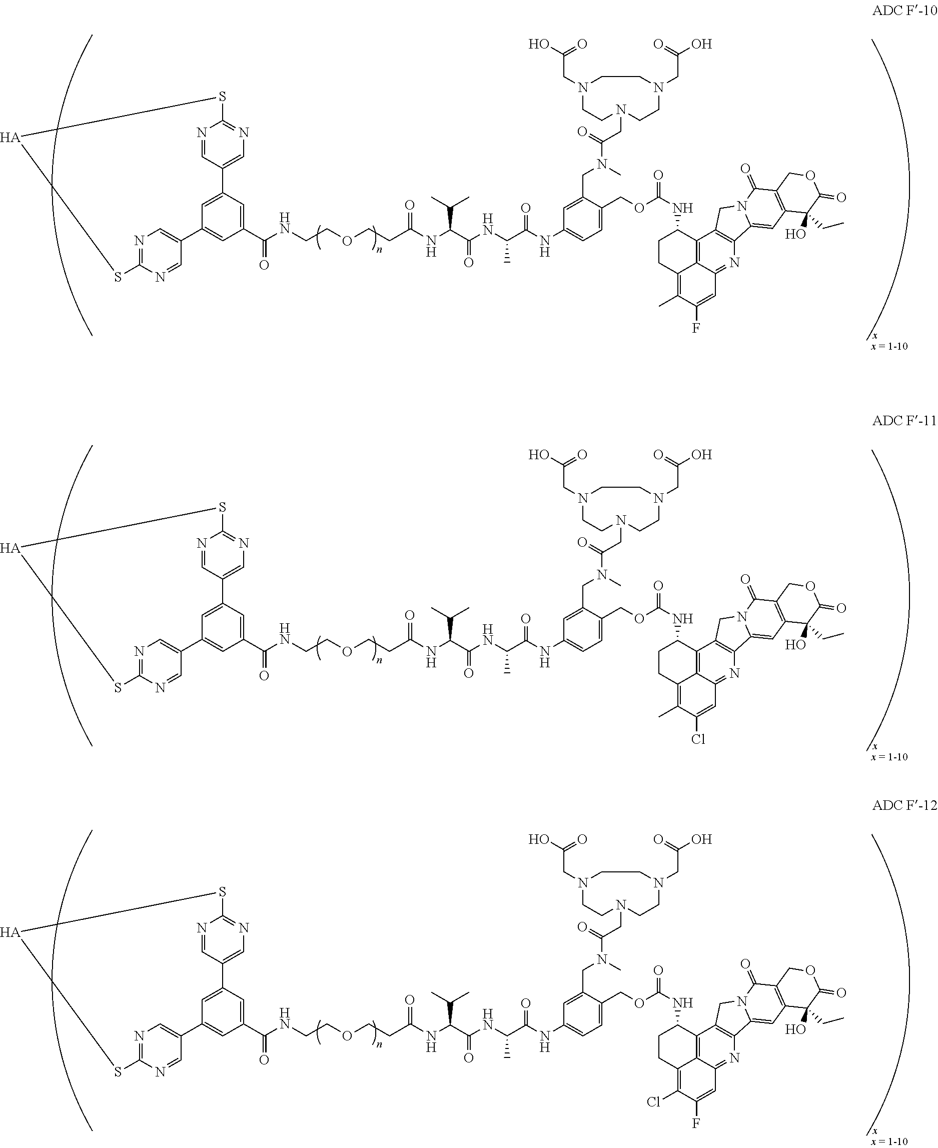

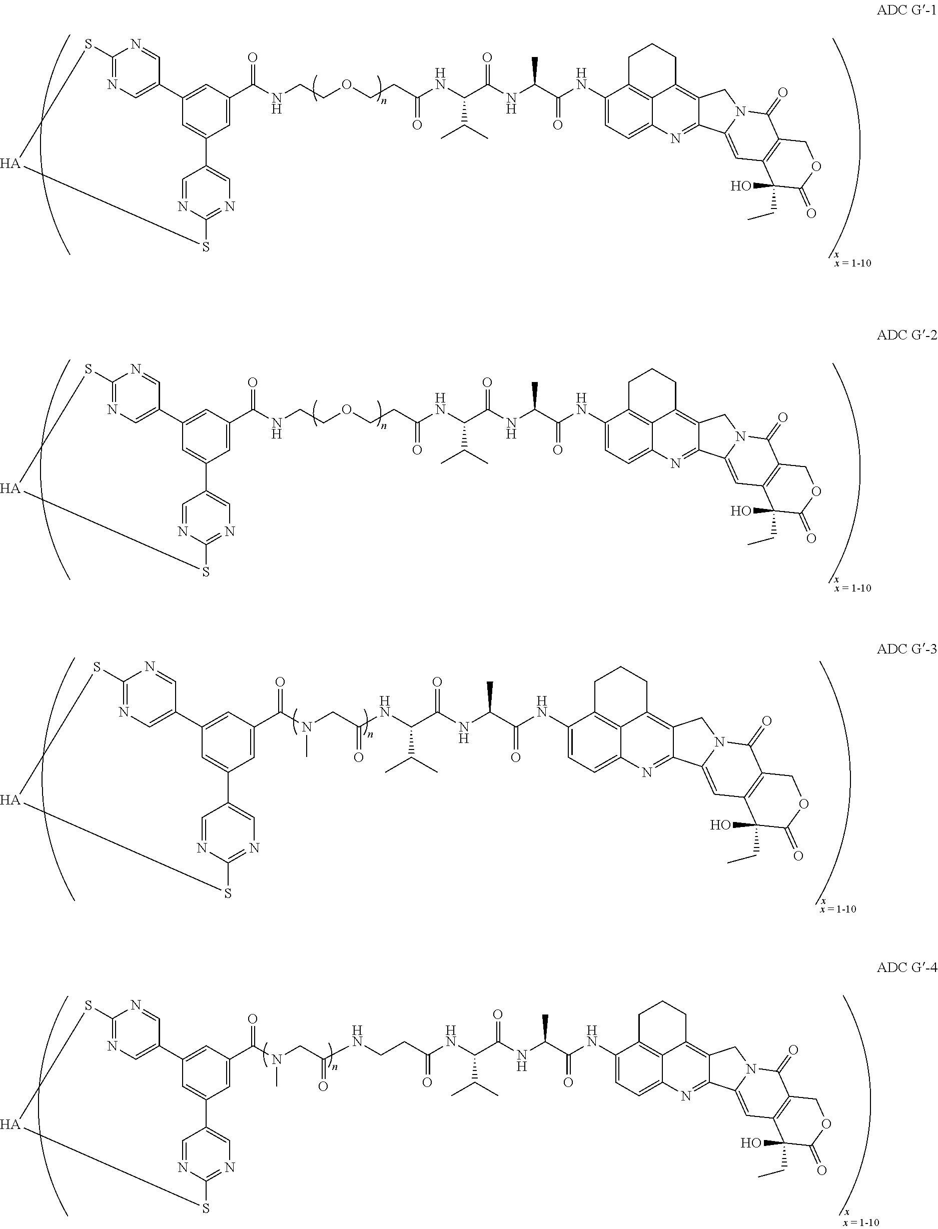

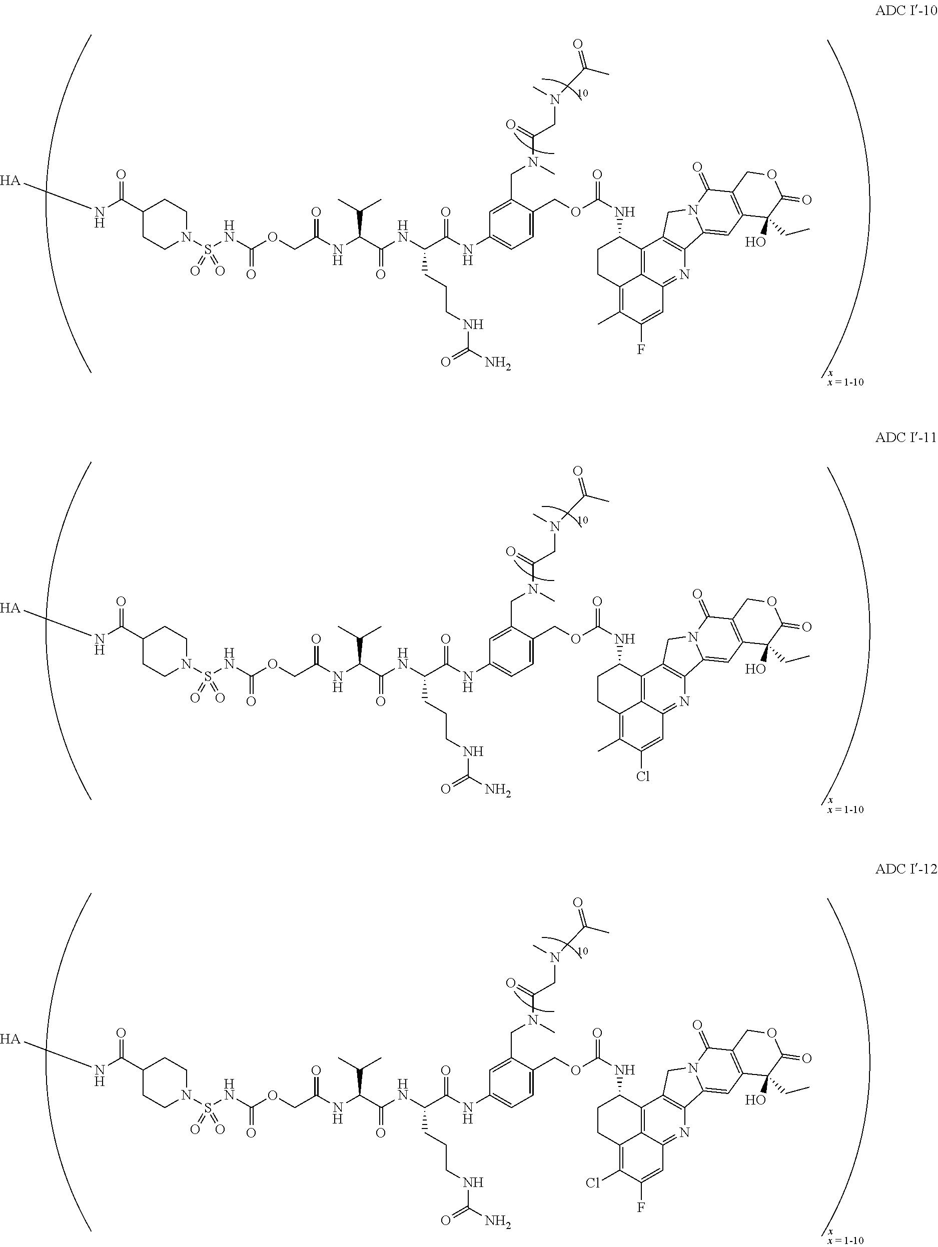

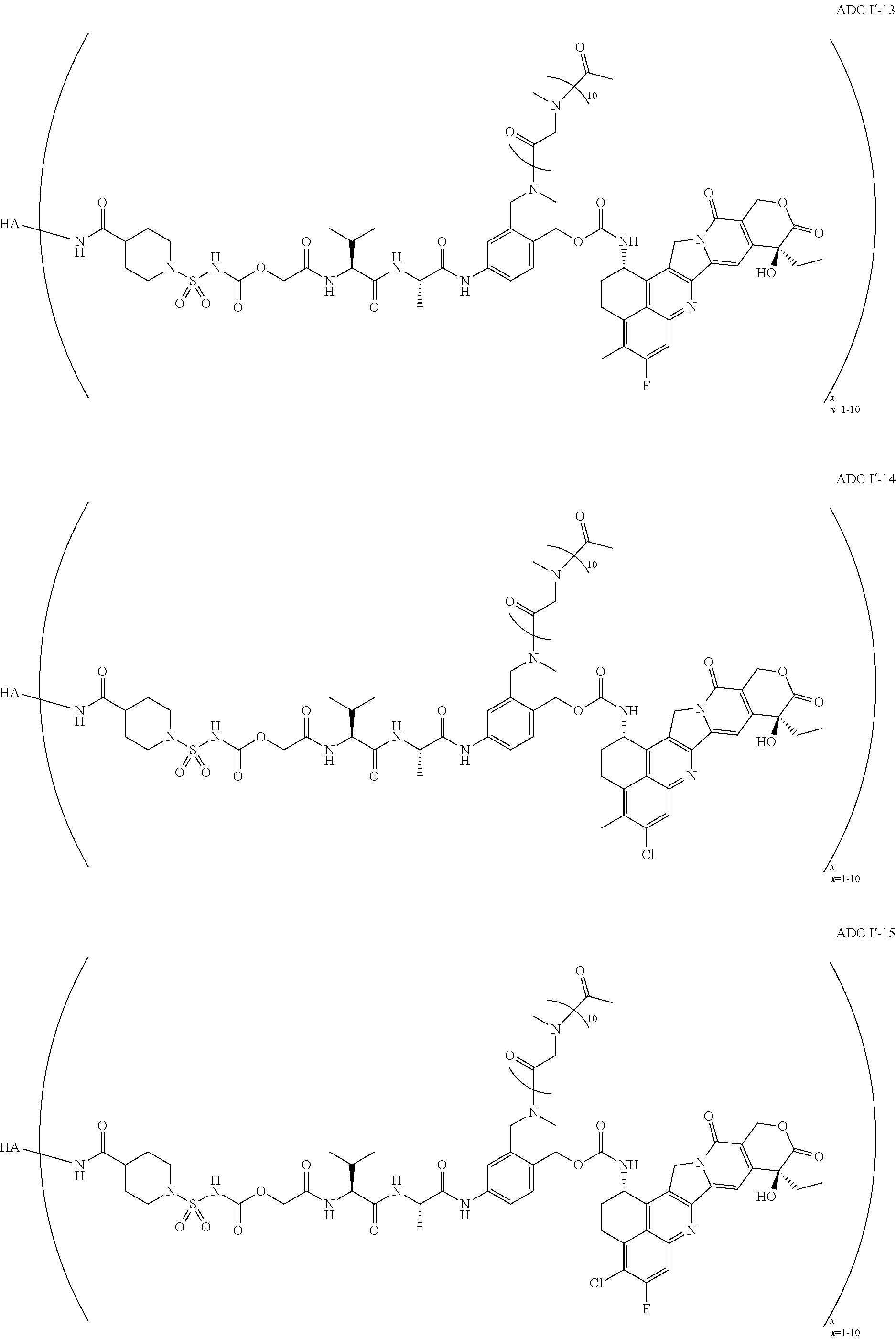

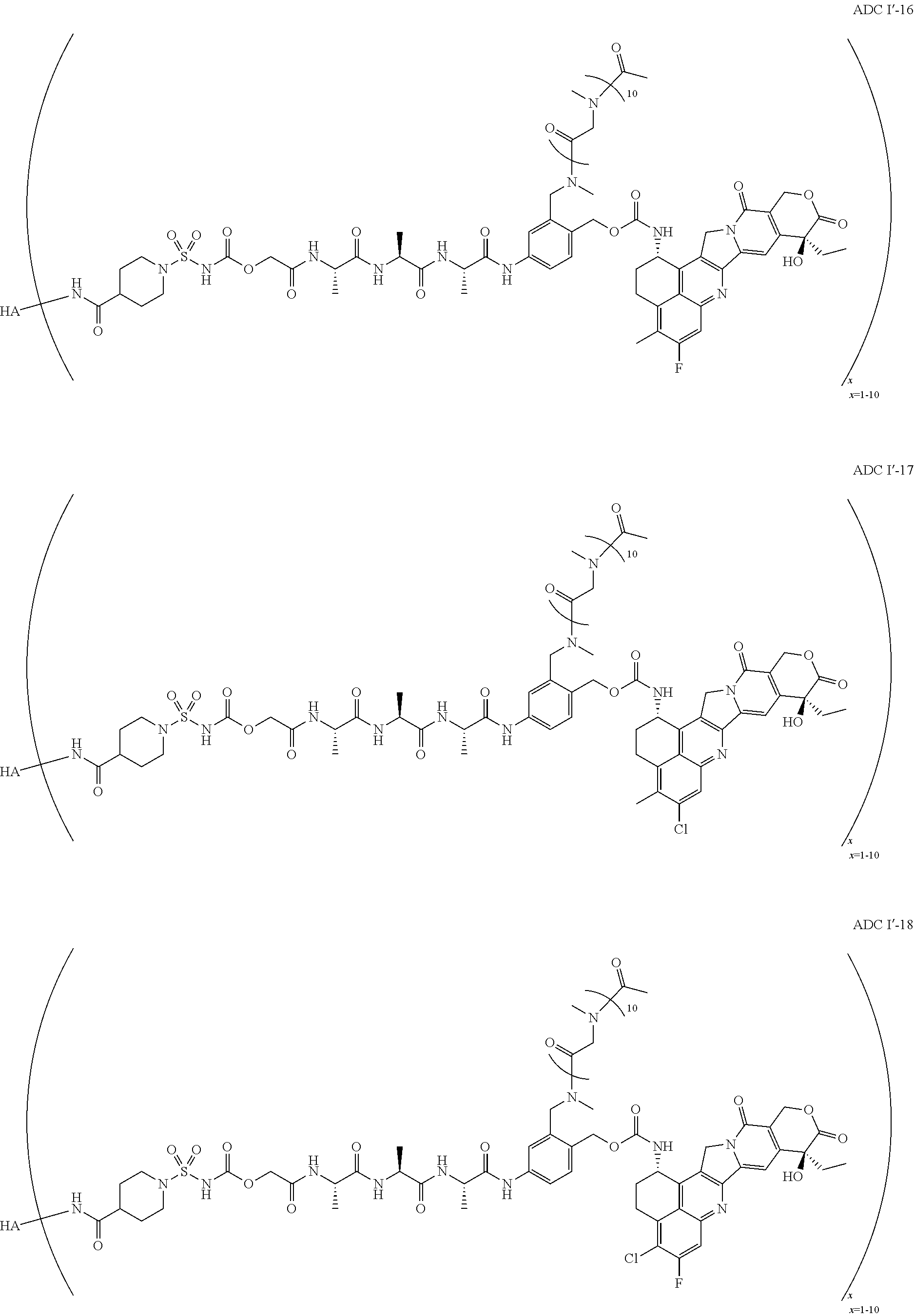

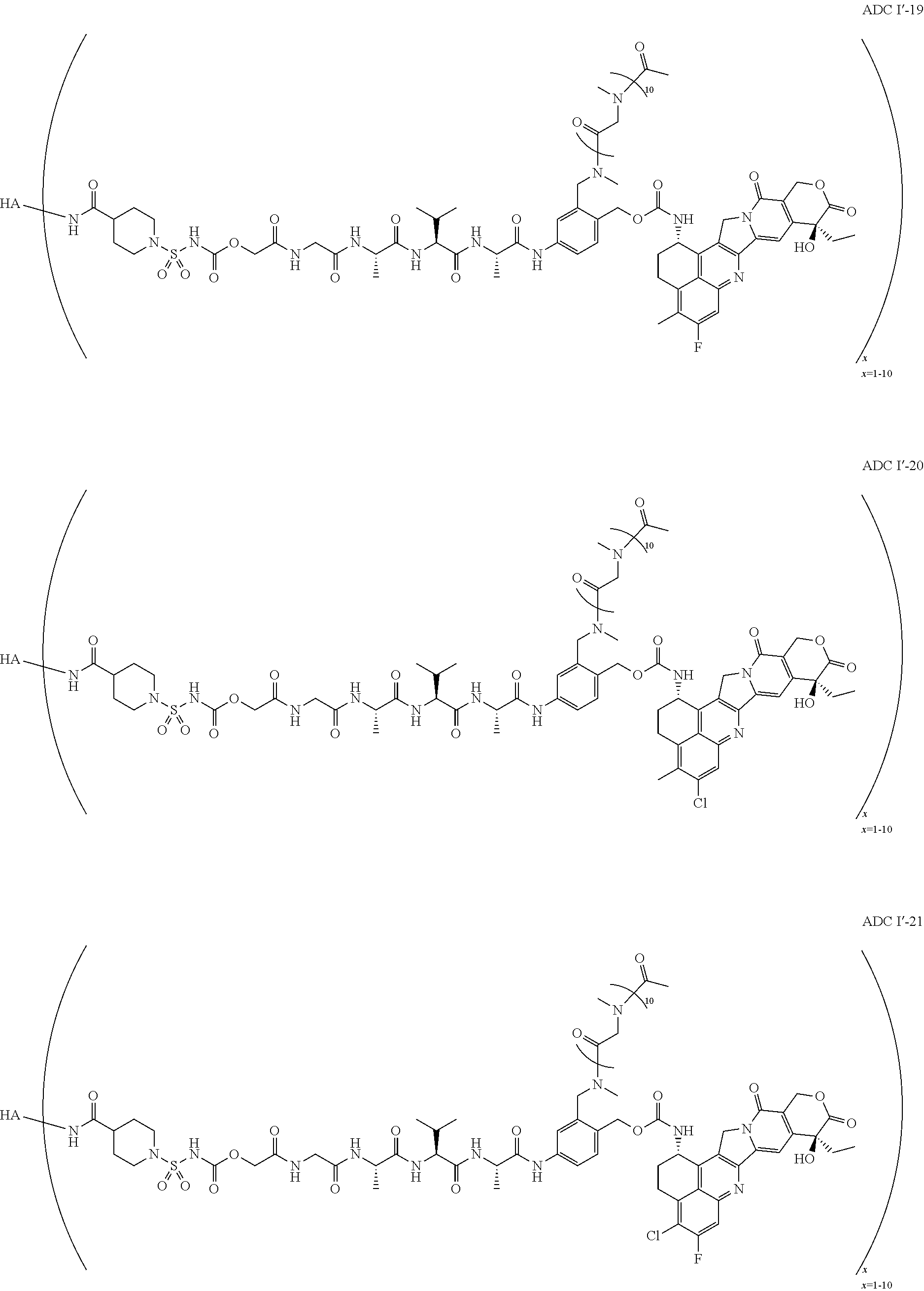

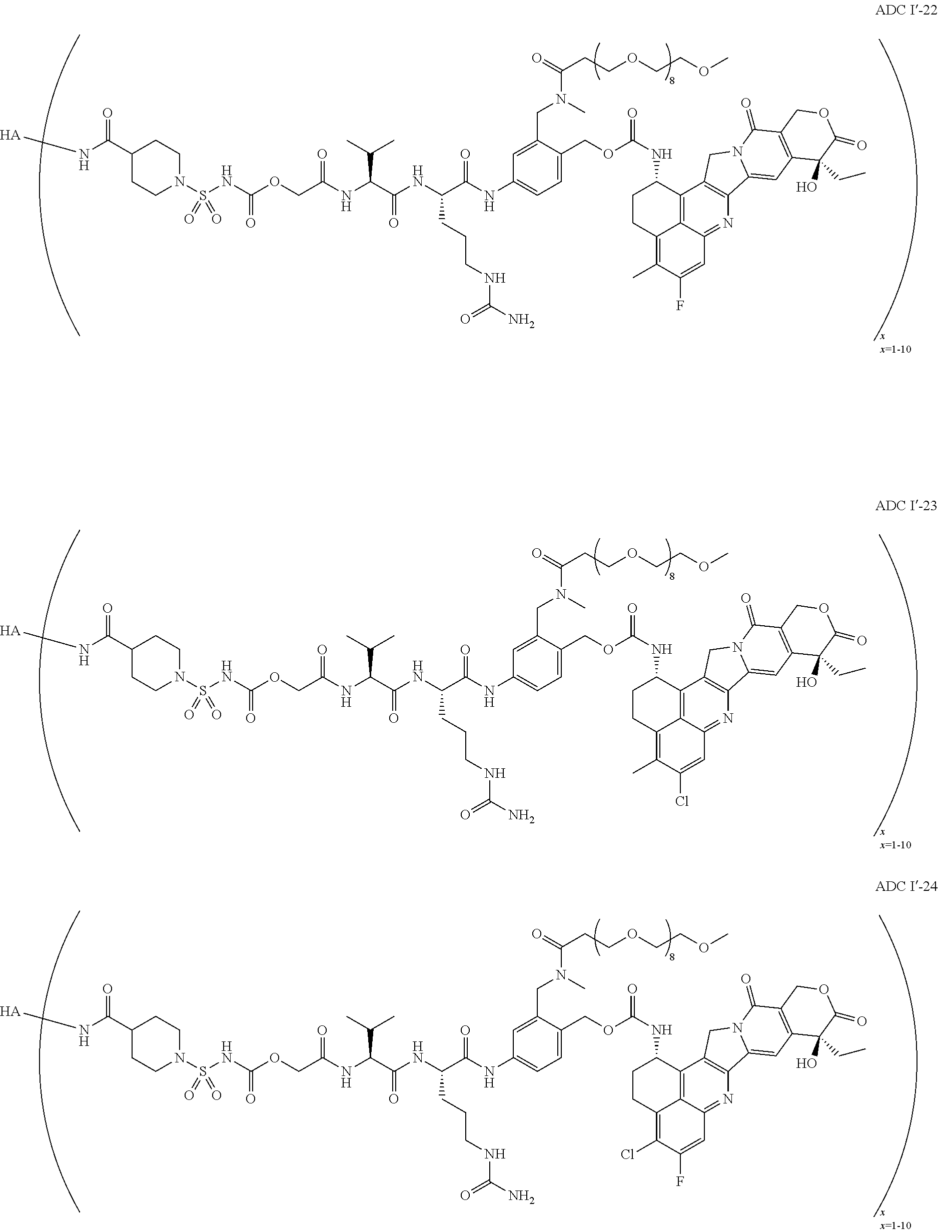

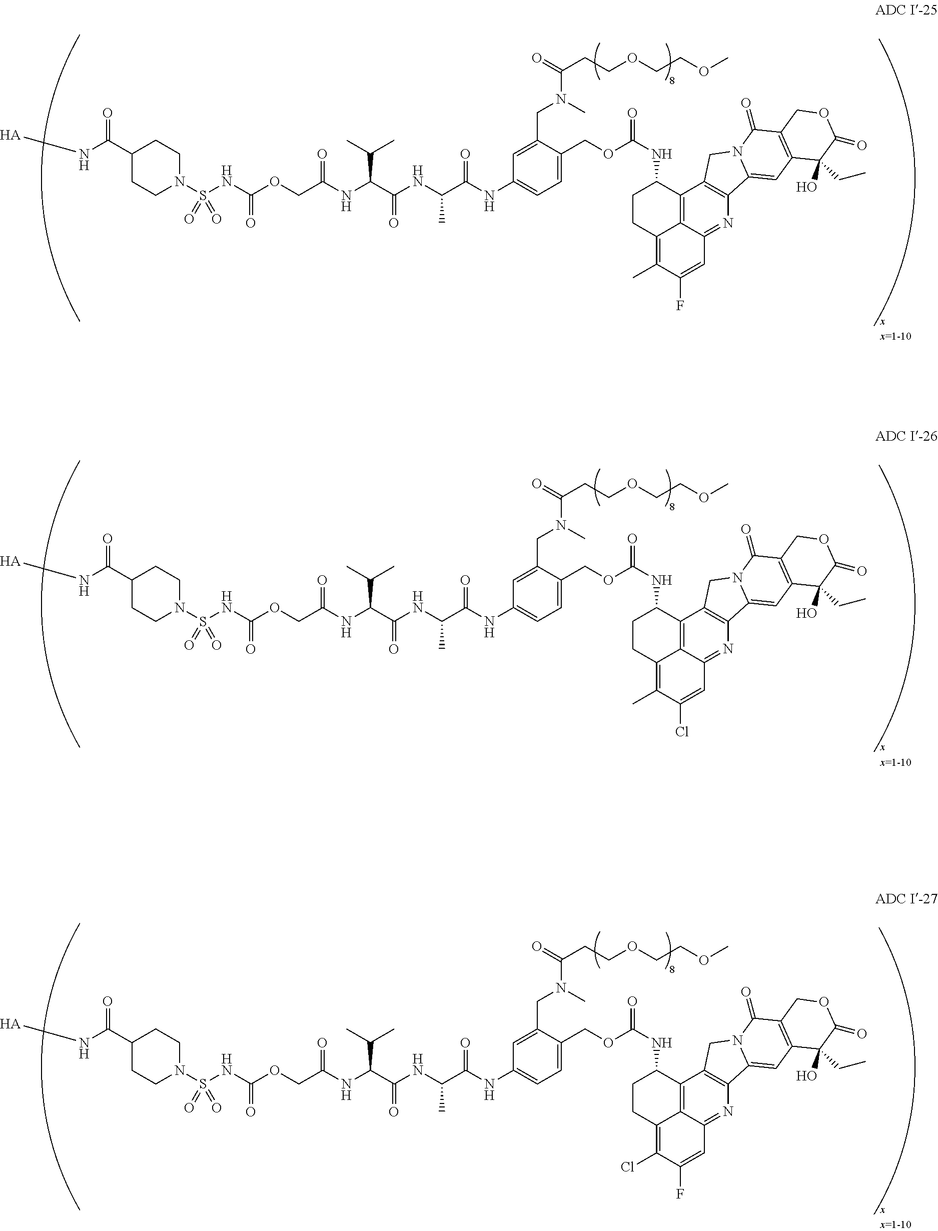

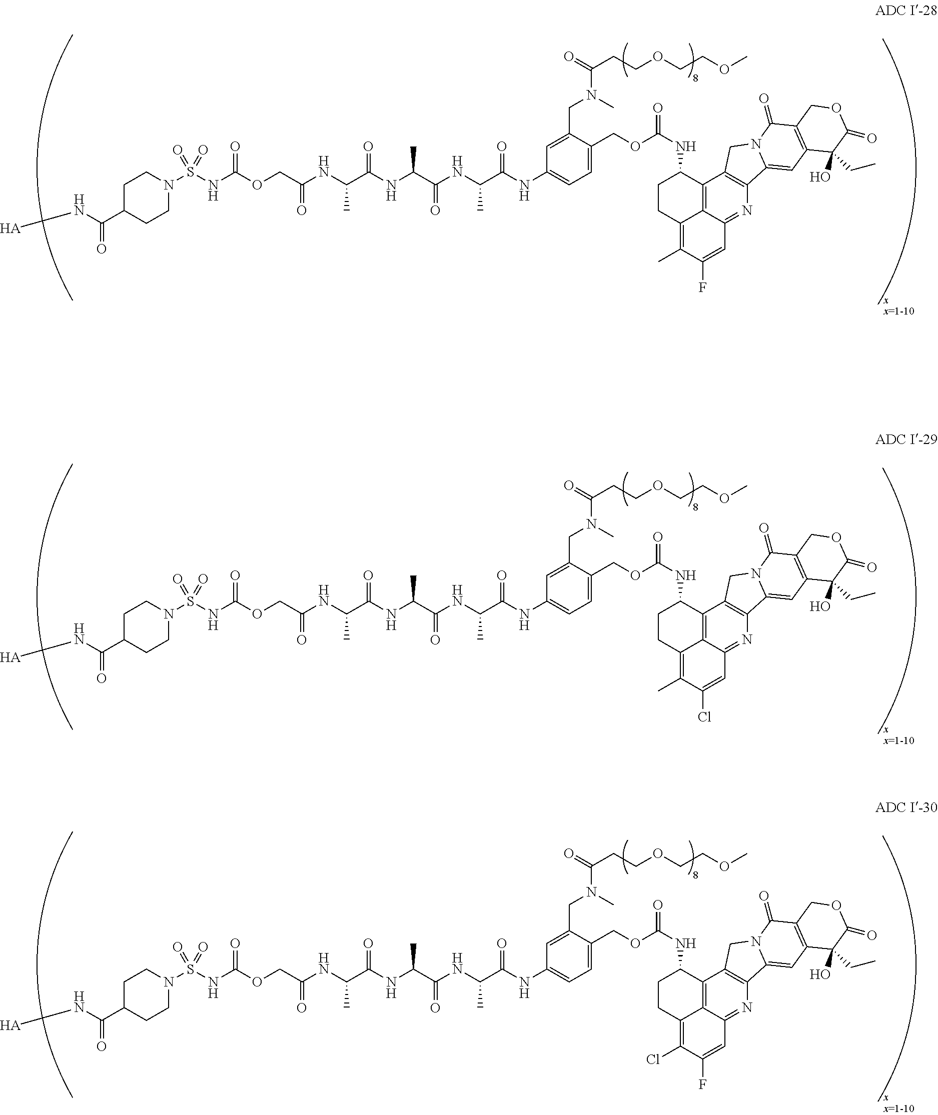

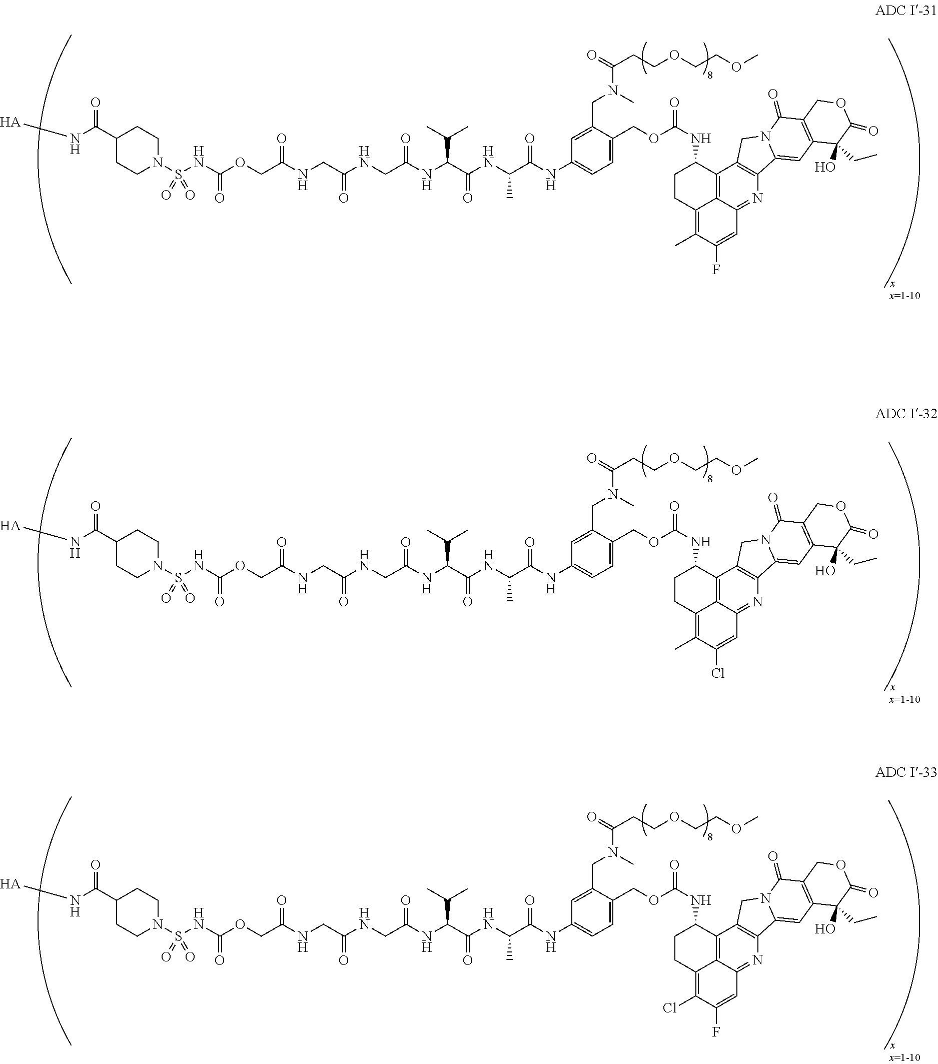

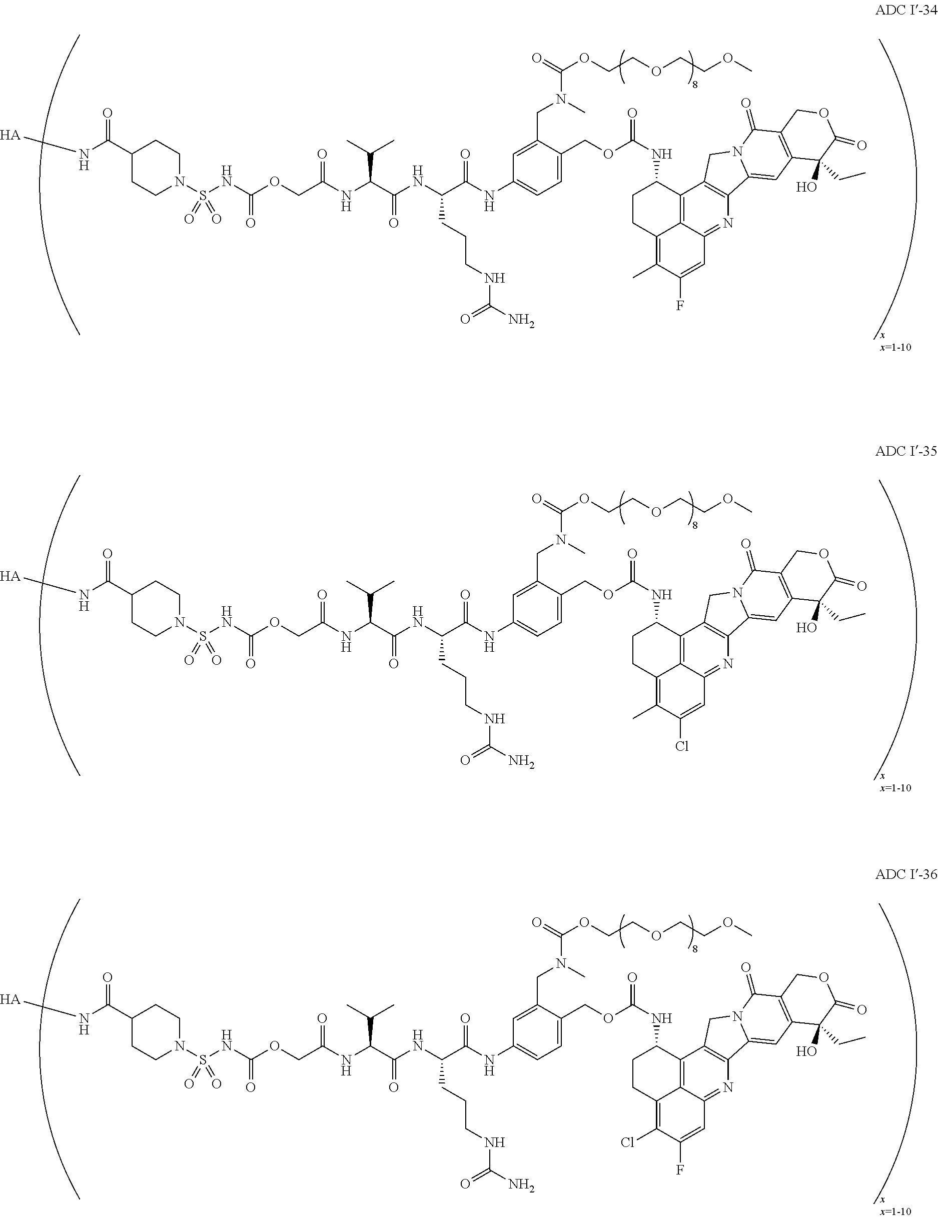

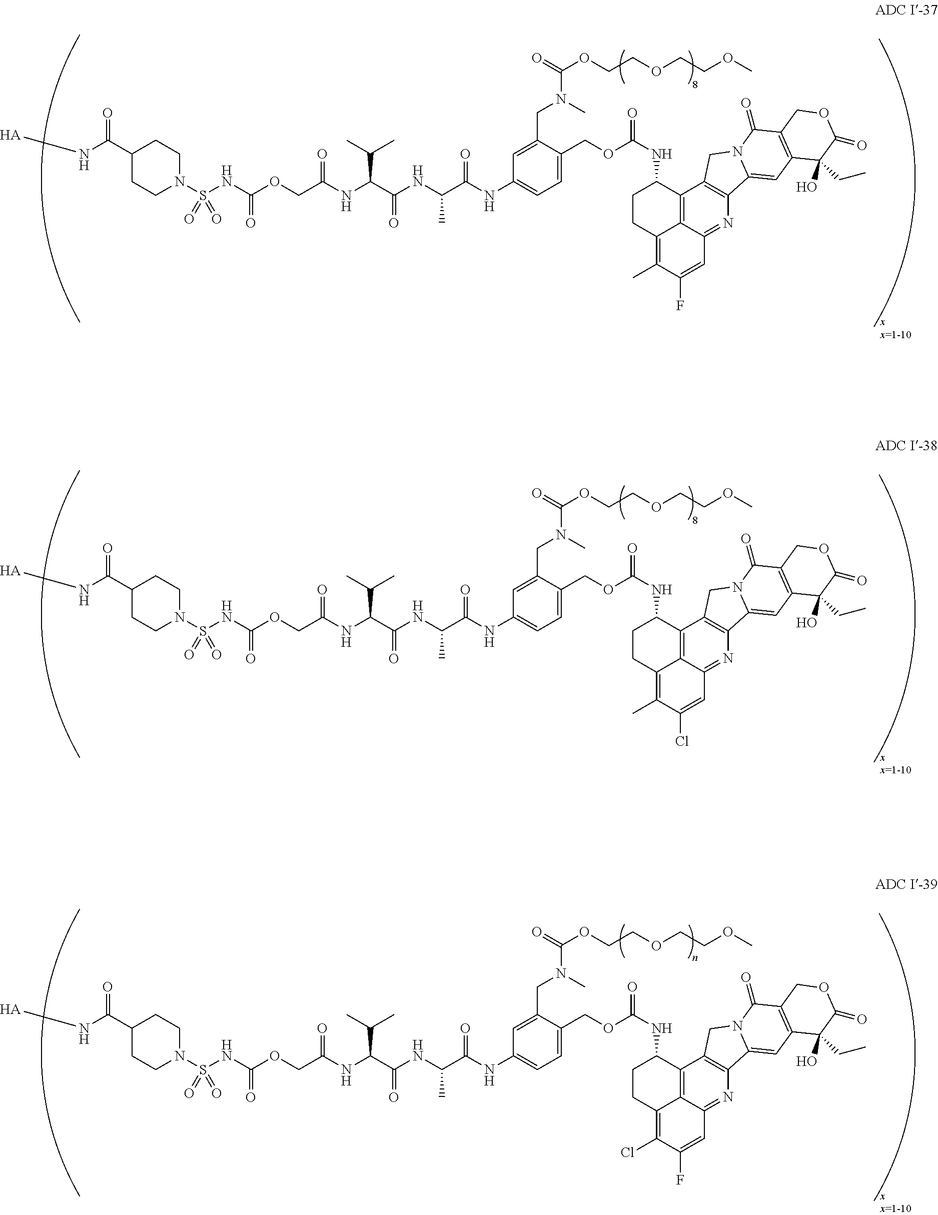

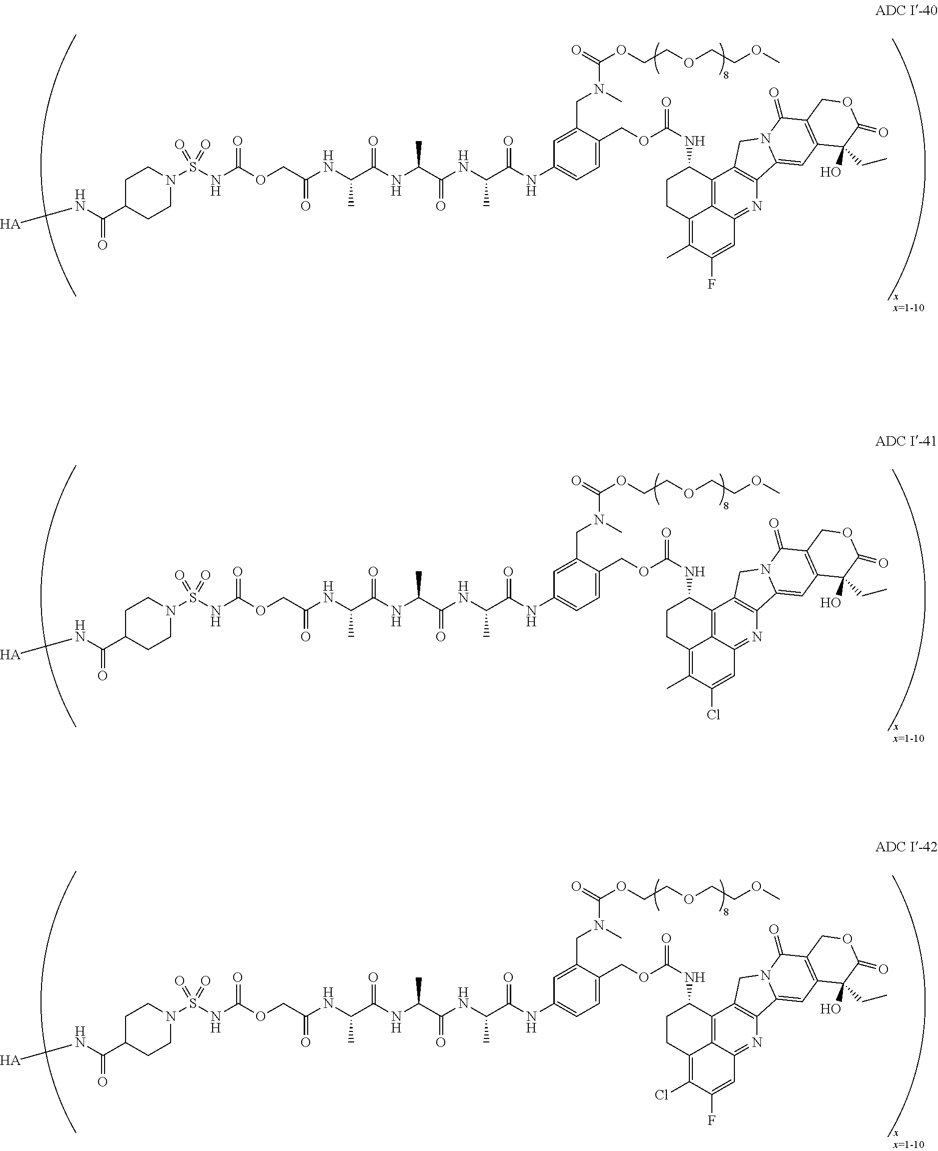

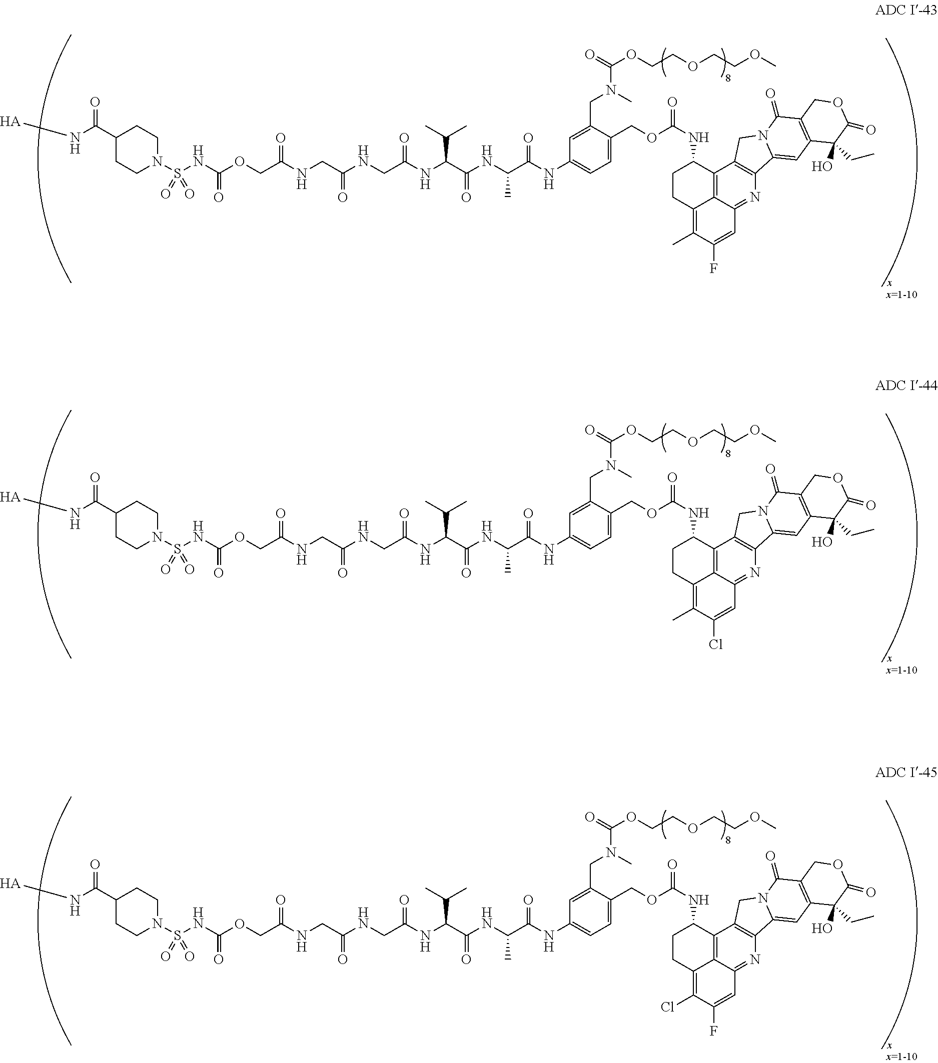

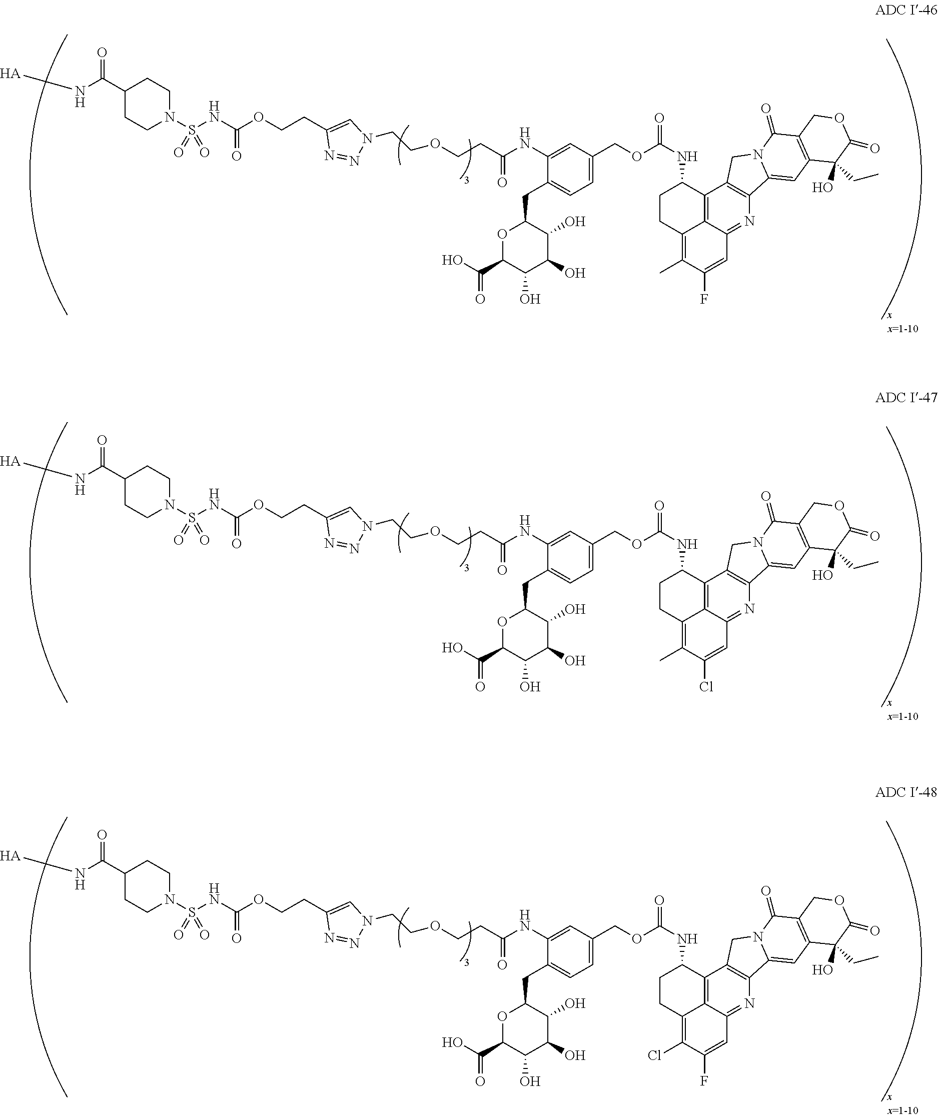

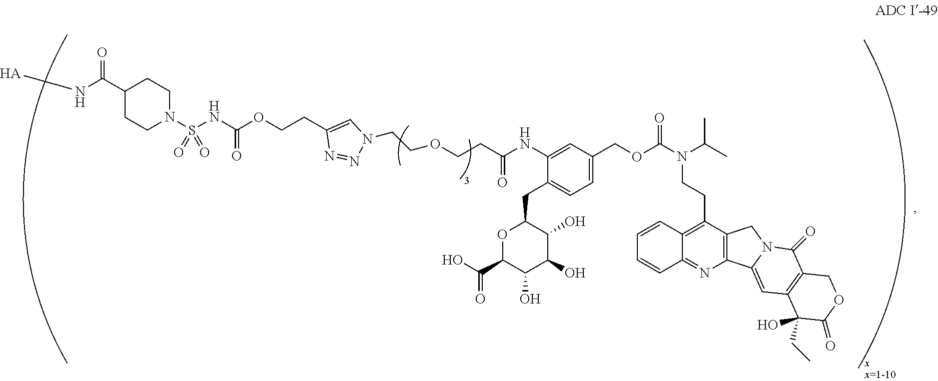

wherein, HA in each of the antibody-drug conjugate represents an antibody or antigen-binding fragment thereof capable of specifically binding to epidermal growth factor receptor 2 (Her2), a member of the ErbB family receptor tyrosine kinase; n is an integer selected from 1 to 20, such as an integer of 1 to 12, 3 to 12, for example, n is 1, 2, 3, 4, 5, 6, 7, 8, 9, 10, 11, 12, 13, 14 or 15;
wherein,

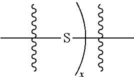 [SMILES: *S*]

represents a specific linkage between a sulfhydryl group in the antibody or antigen-binding fragment thereof and the M fragment;

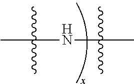 [SMILES: *N*]

represents a specific linkage between an amino group in the antibody or antigen-binding fragment thereof and the M fragment.

In some embodiments, the antibody or antigen-binding fragment thereof is as defined above.

In some embodiments, HA in each antibody-drug conjugate represents Trastuzumab, Pertuzumab, or antigen-binding fragment thereof.

In some embodiments, HA in each antibody-drug conjugate comprises represents following antibody or antigen-binding fragment:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36.

In some embodiments, x in the antibody-drug conjugate represented by Ab-[M-L-E-D]ₓ is 1 to 10, such as 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10 or 9 to 10.

In some embodiments, x in the antibody-drug conjugate represented by Ab-[M-L-E-D]ₓ is 1, 2, 3, 4, 5, 6, 7, 8, 9 or 10. In some embodiments, x in the antibody-drug conjugate represented by Ab-[M-L-E-D]ₓ is about 2. In some embodiments, x in the antibody-drug conjugate represented by Ab-[M-L-E-D]ₓ is about 4.

The antibody-drug conjugate described in the present invention is optionally substituted with one or more suitable substituents.

### Intermediates

In some embodiments, the present application provides intermediate compounds having the following structures or salts, stereoisomers, tautomers or isotopically labeled compounds thereof:

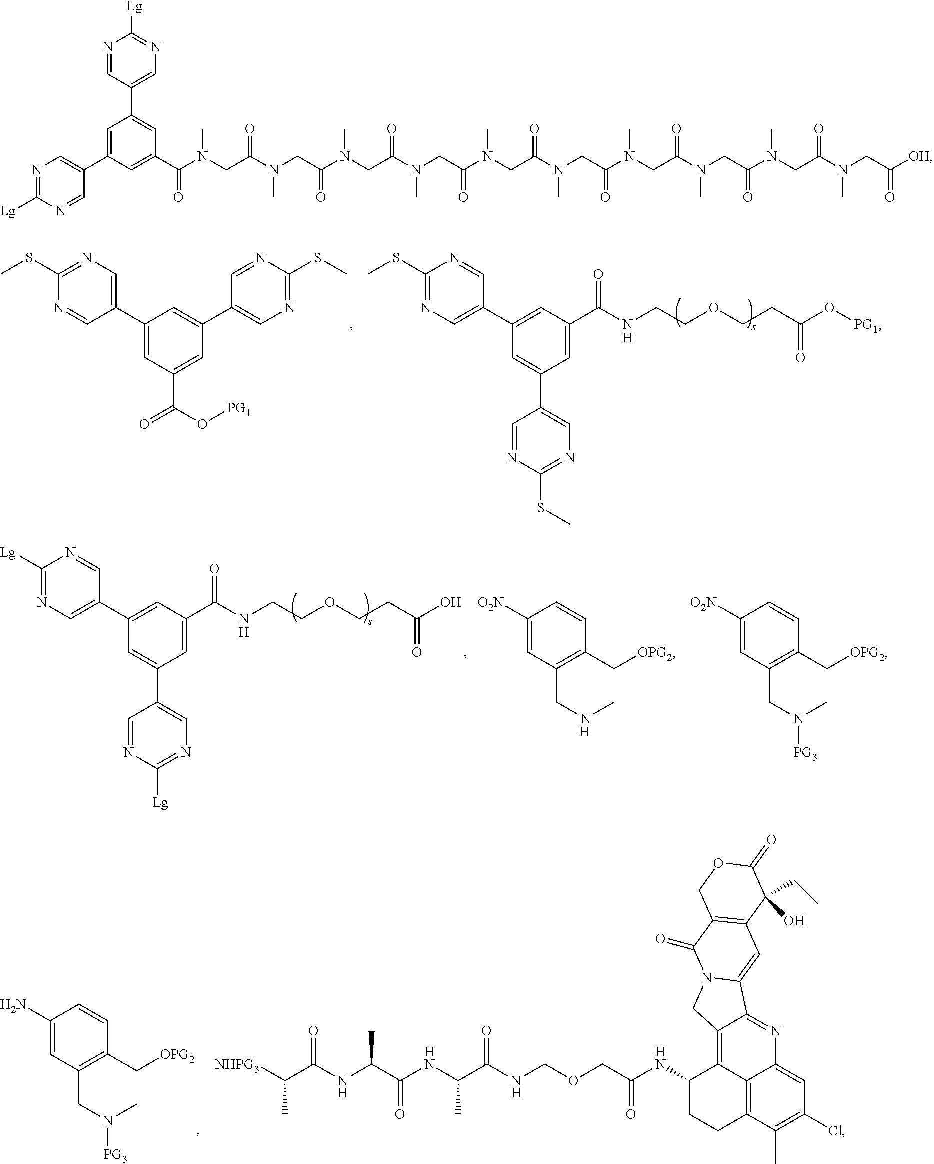

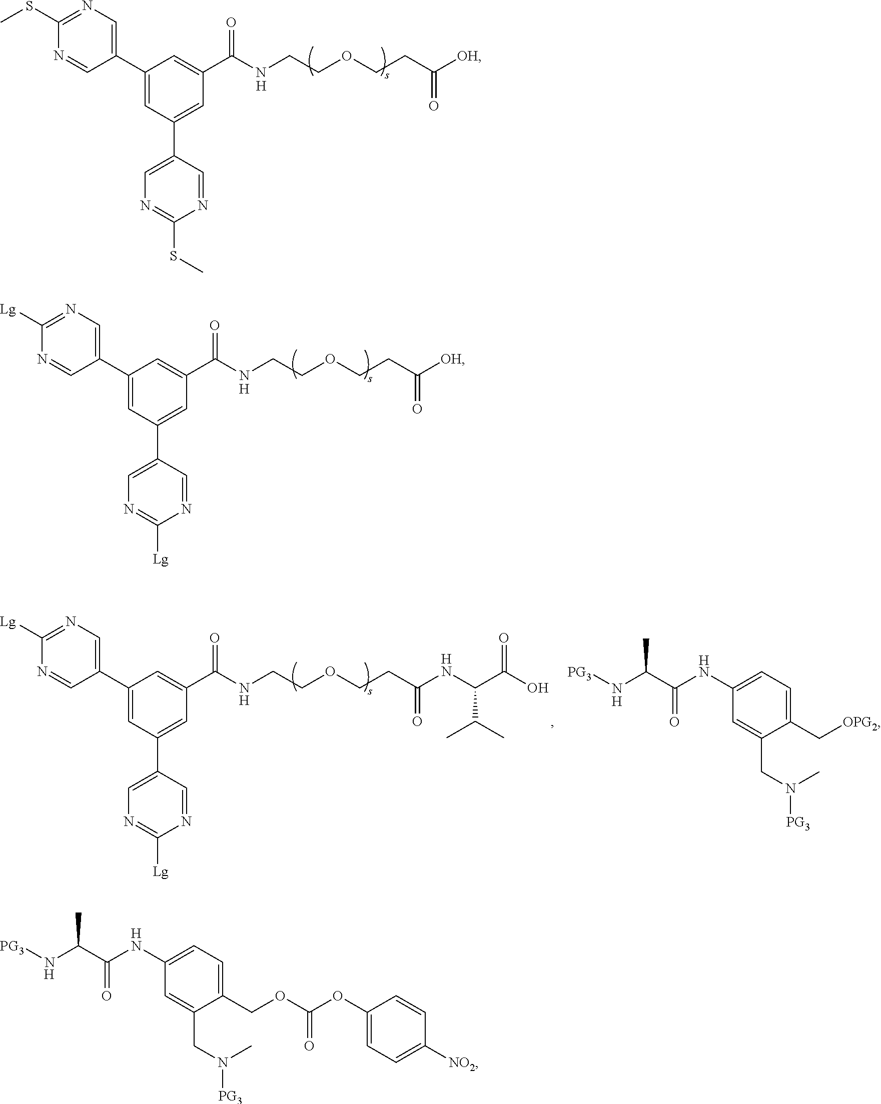

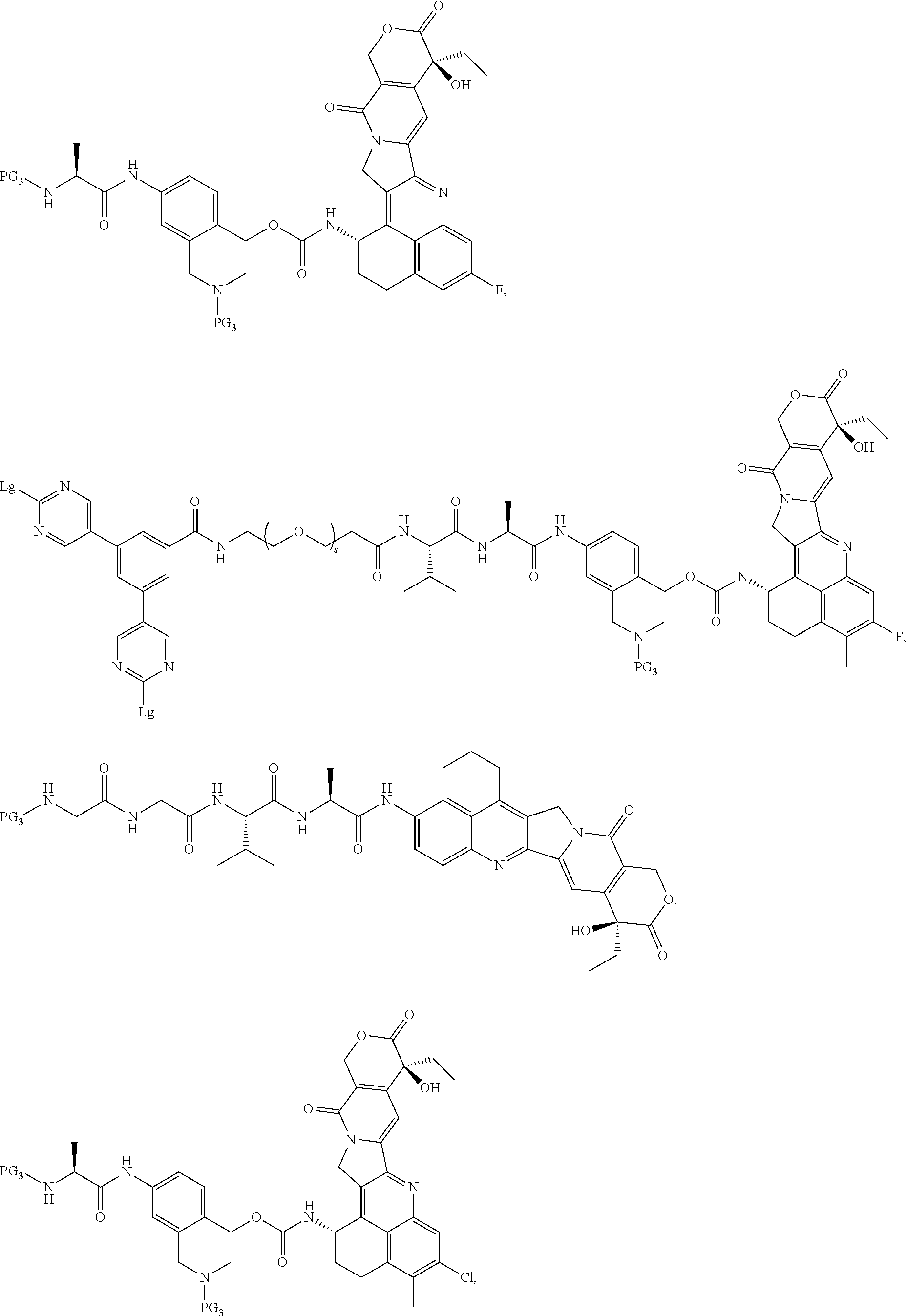

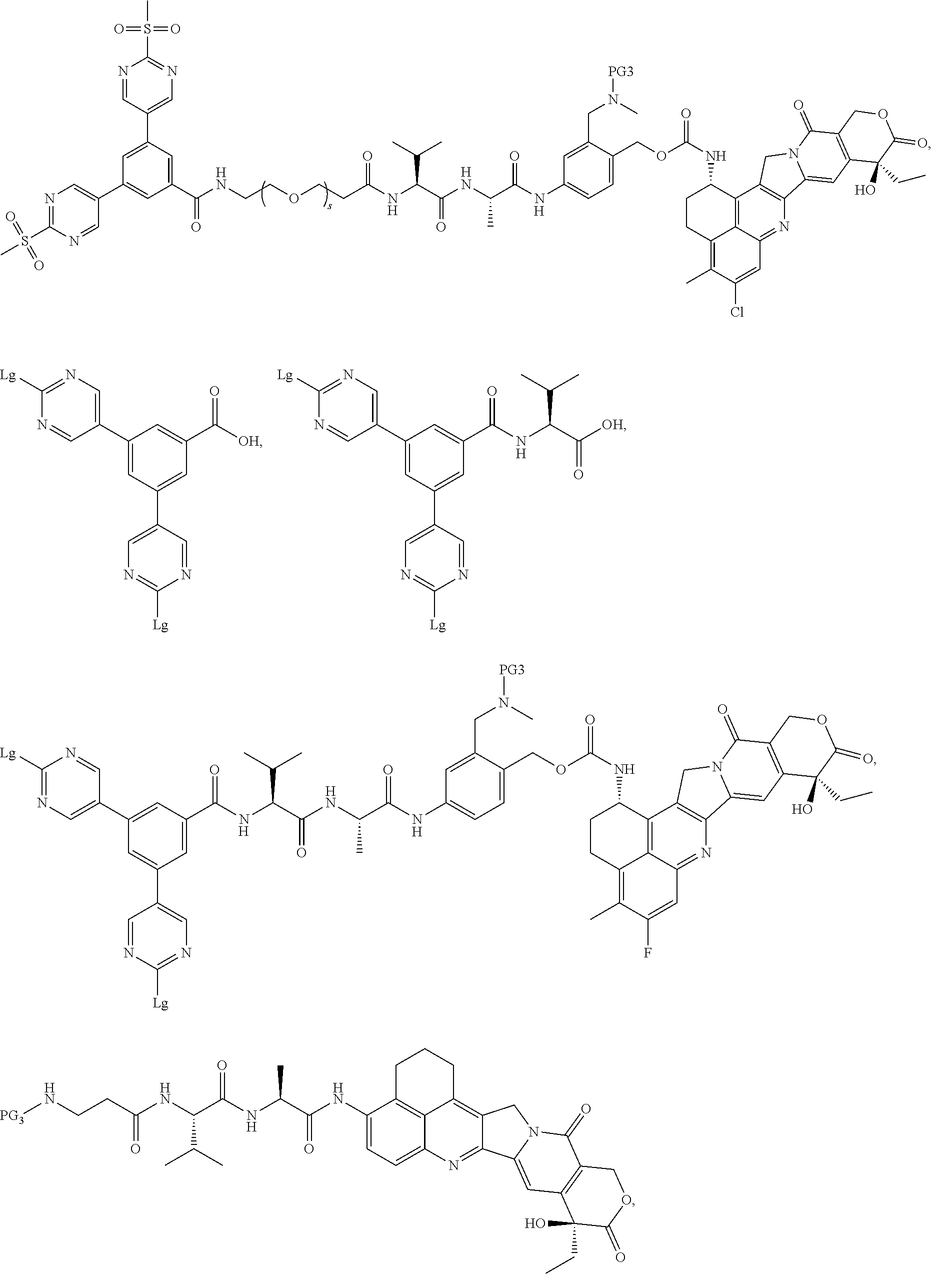

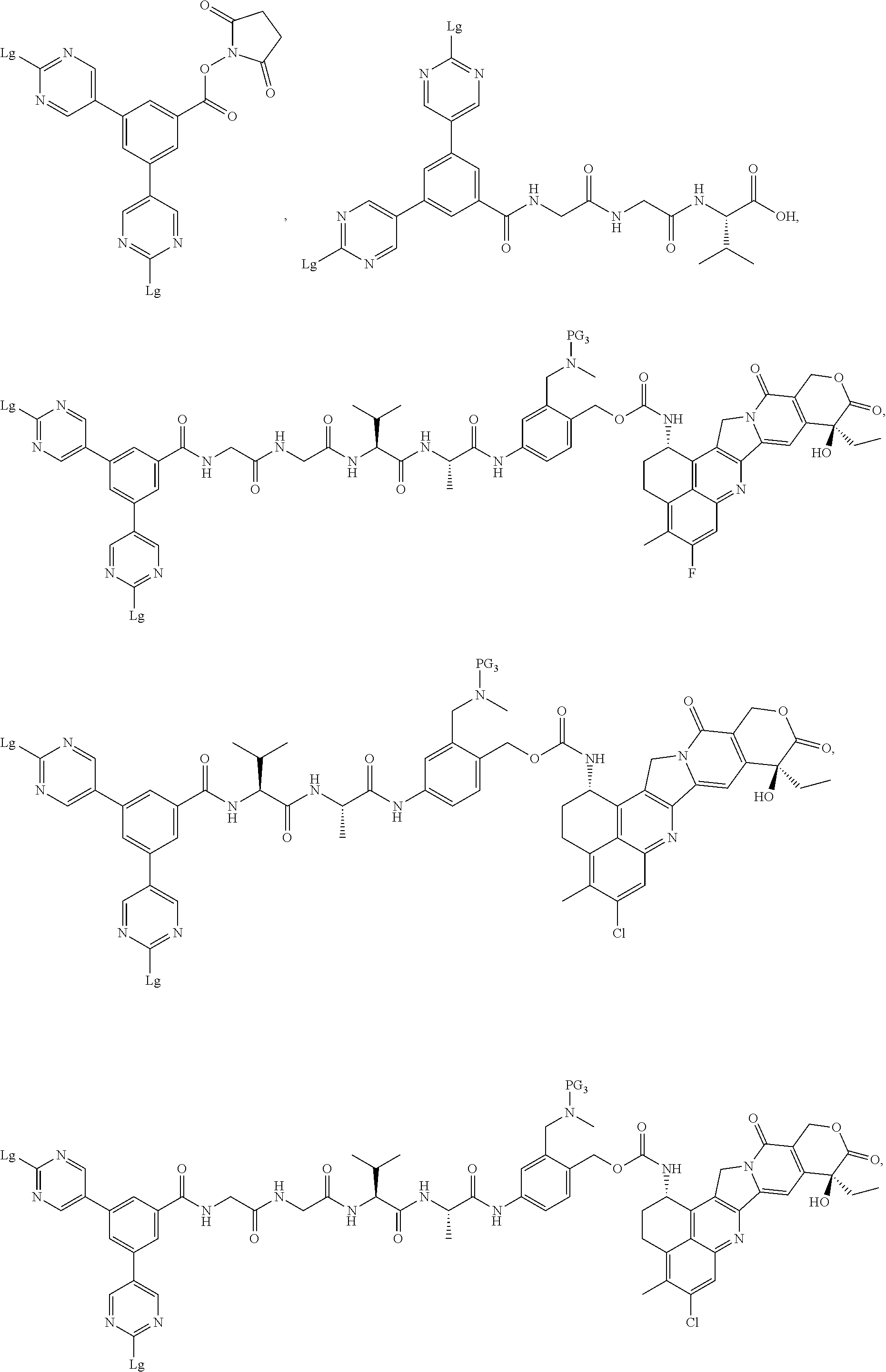

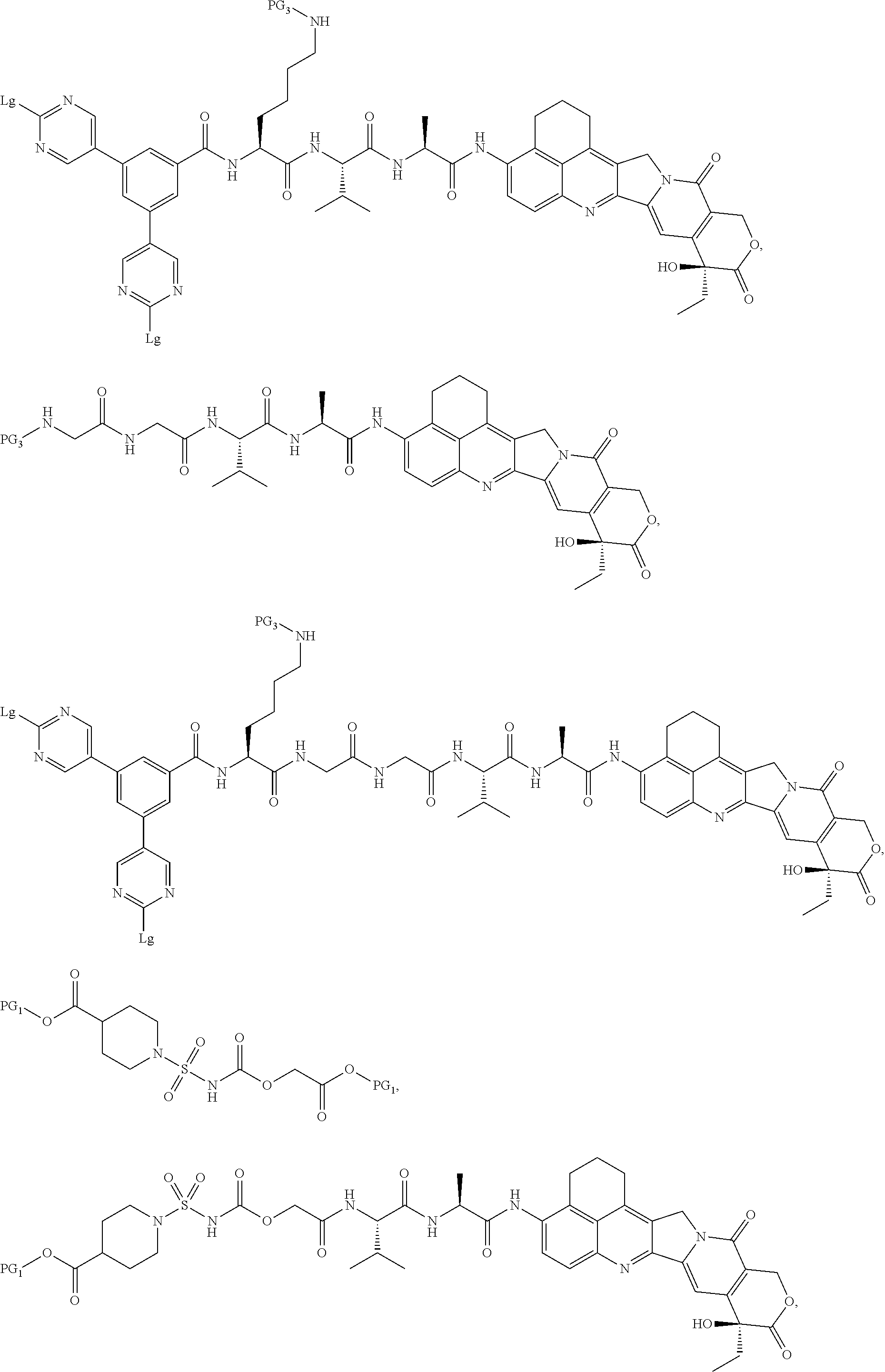

wherein
PG₁ is each independently H or a carboxyl-protecting group, and the carboxyl-protecting group is, for example, C₁₋₆ alkyl, allyl, benzyl, 2,4-dimethoxybenzyl, p-methoxybenzyl, methoxyethoxymethyl, pentafluorophenyl, 4-p-methylbenzyloxybenzyl;
PG₂ is each independently H or a hydroxyl-protecting group, and the hydroxyl-protecting group is, for example, trimethylsilyl (TMS), triethylsilyl (TES), triisopropylsilyl (TIPS), tert-butyldimethylsilyl (TBS), tert-butyldiphenylsilyl (TBDPS), methyl, tert-butyl, allyl, benzyl, methoxymethyl (MOM), ethoxyethyl, 2-tetrahydropyranyl (THP), formyl, acetyl, benzoyl, or p-nitrobenzoyl;
PG₃ is each independently H or an amino-protecting group, and the amino-protecting group is an alkoxycarbonyl amino-protecting group, such as benzyloxycarbonyl group (Cbz), tert-butyloxycarbonyl group (Boc), fluorenylmethyloxycarbonyl group (Fmoc), allyloxycarbonyl group (Alloc), trimethylsilylethoxycarbonyl group (Teoc), methyl(or ethyl)oxycarbonyl group; an acyl amino-protecting group, such as phthaloyl (Pht), p-toluenesulfonyl (Tos), trifluoroacetyl (Tfa), o-(p-)nitrobenzenesulfonyl (Ns), pivaloyl, benzoyl, tert-butyloxycarbonyl group, 9-fluorenylmethoxycarbonyl group, allyloxycarbonyl group, trichloroethoxycarbonyl group, trimethylsilylethoxycarbonyl group, benzyloxycarbonyl group, p-toluenesulfonyl, p-nitrobenzenesulfonyl, trifluoroacetyl, methoxycarbonyl group, or ethoxycarbonyl; an alkyl amino-protecting group, such as trityl (Trt), 2,4-dimethoxybenzyl (Dmb), 4-methoxybenzyl (PMB), benzyl (Bn);
Lg is a leaving group, as defined above;
s is an integer selected from 1 to 20, preferably an integer of 1 to 15, such as an integer of 1 to 12, 3 to 12, such as 1, 2, 3, 4, 5, 6, 7, 8, 9, 10, 11, 12, 13, 14 or 15, preferably 5, 8, 10.

In some embodiments, the present application provides intermediate compounds or salts, stereoisomers, tautomers or isotopically labeled compounds thereof having the structures as shown below:

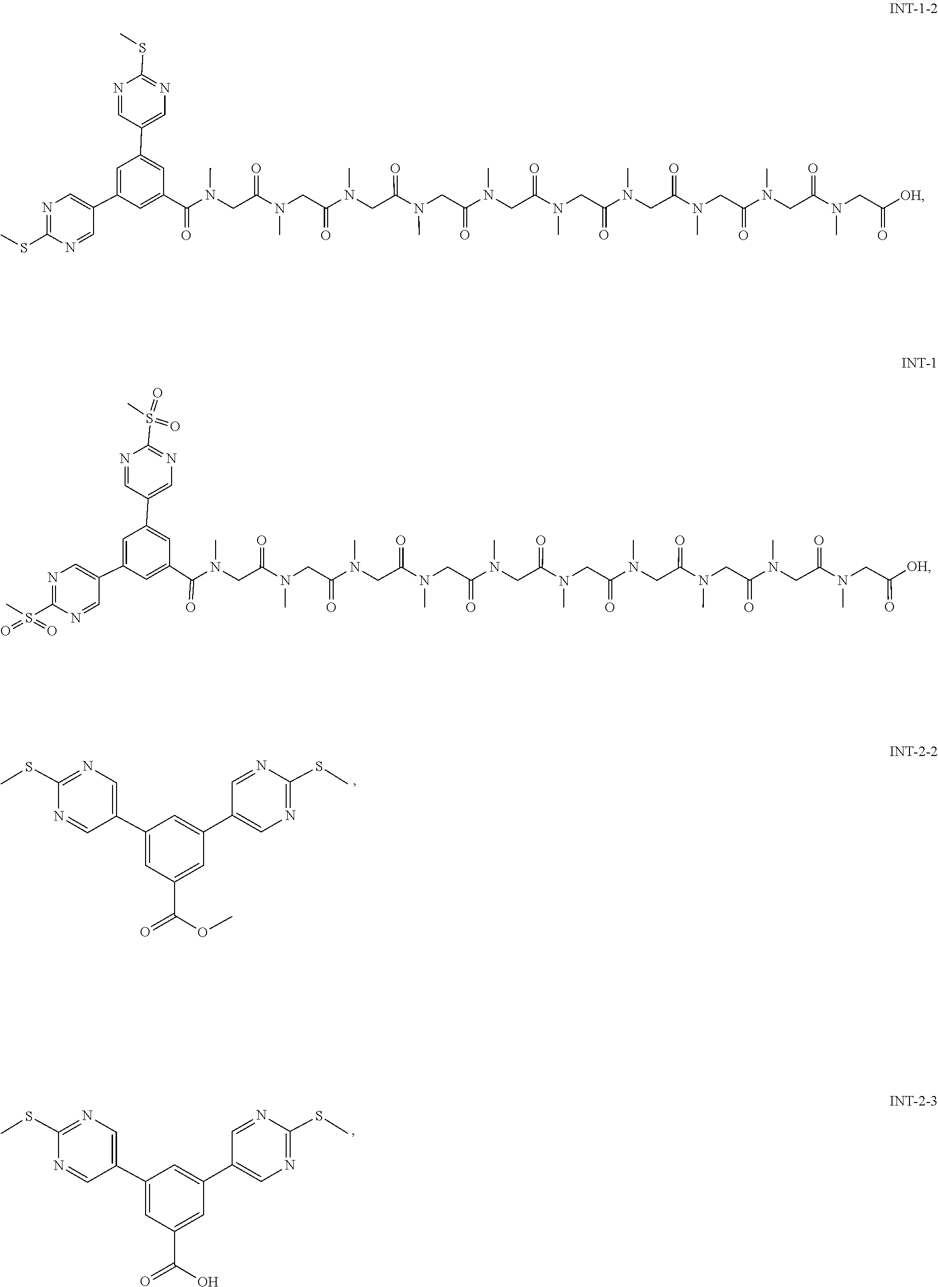

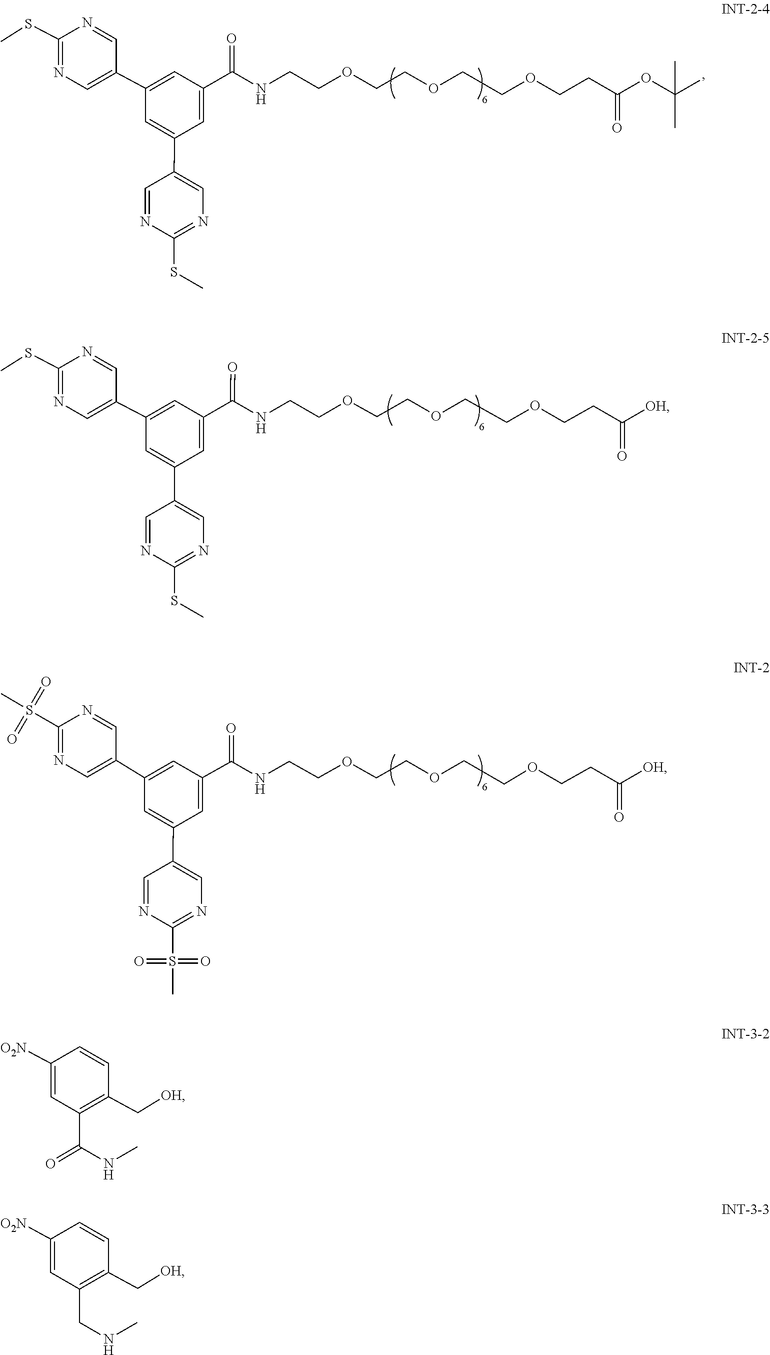

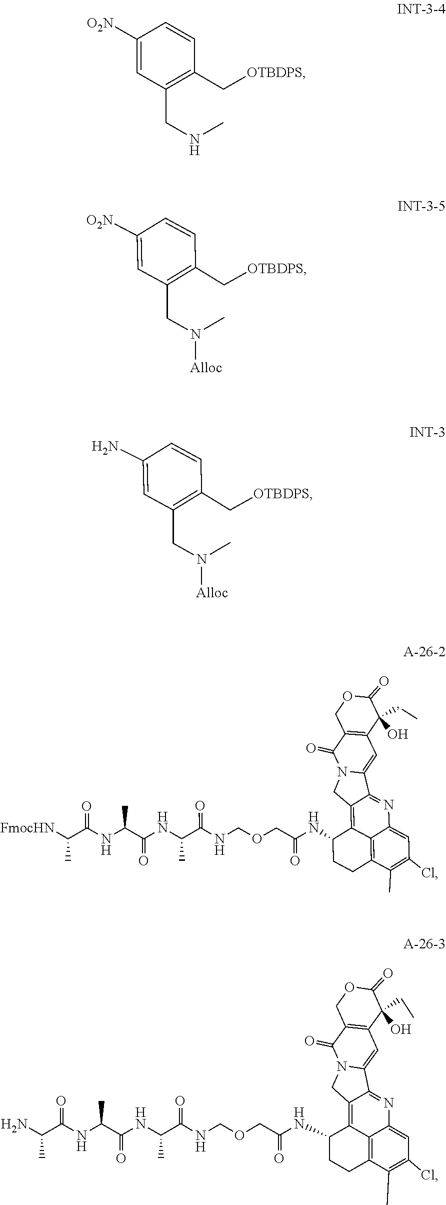

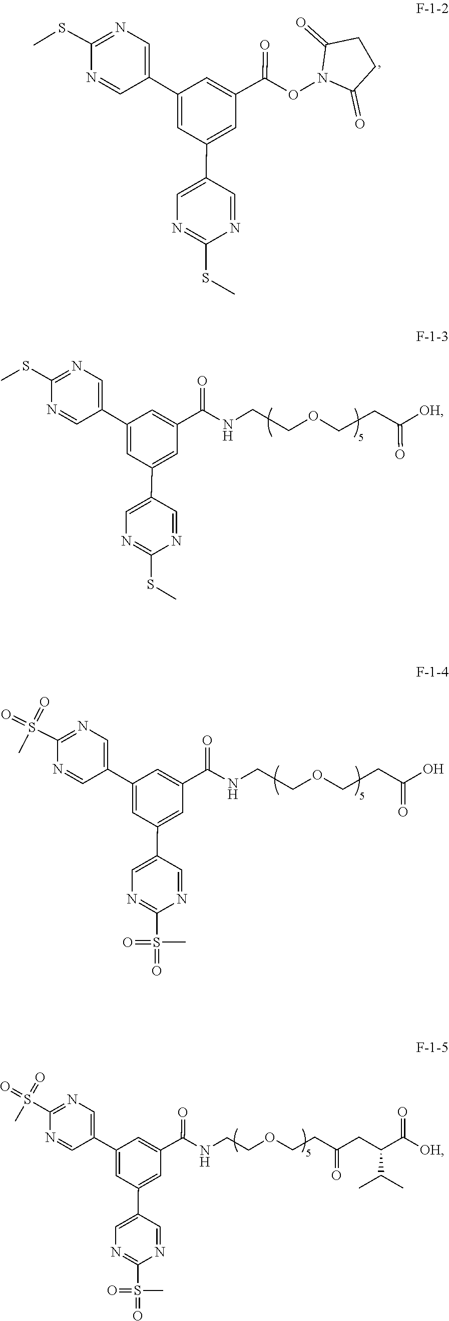

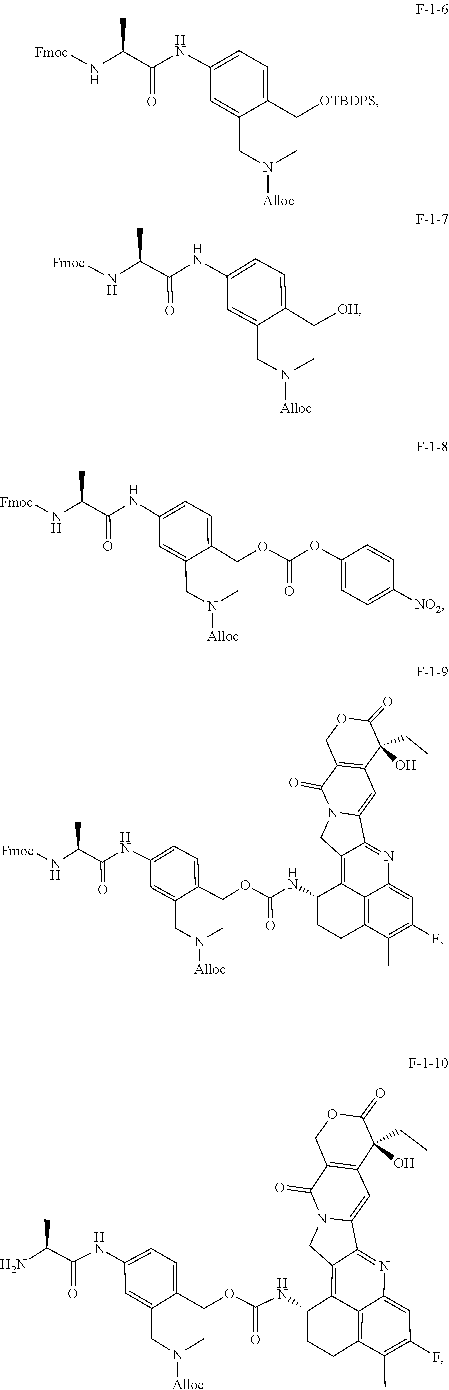

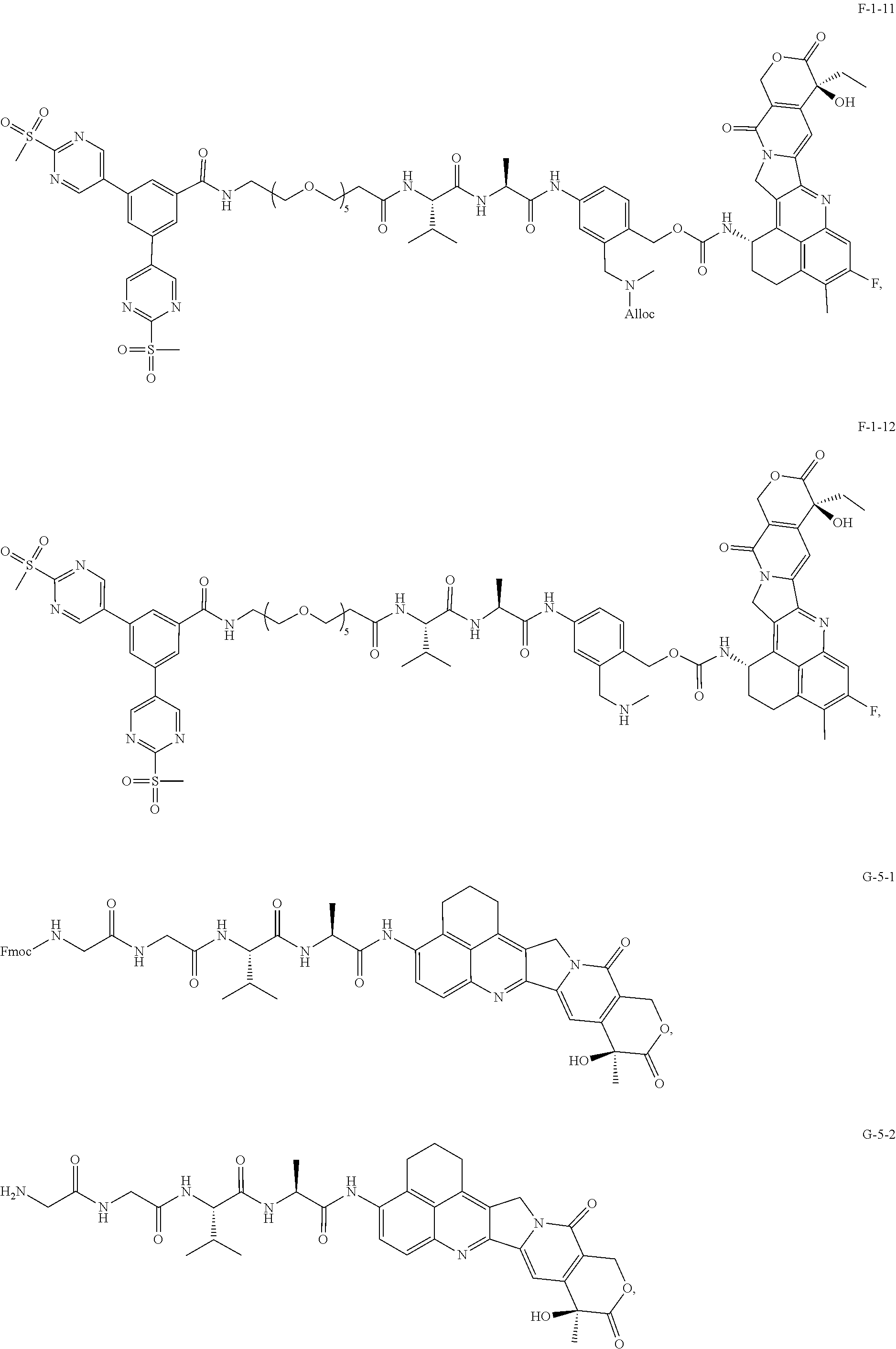

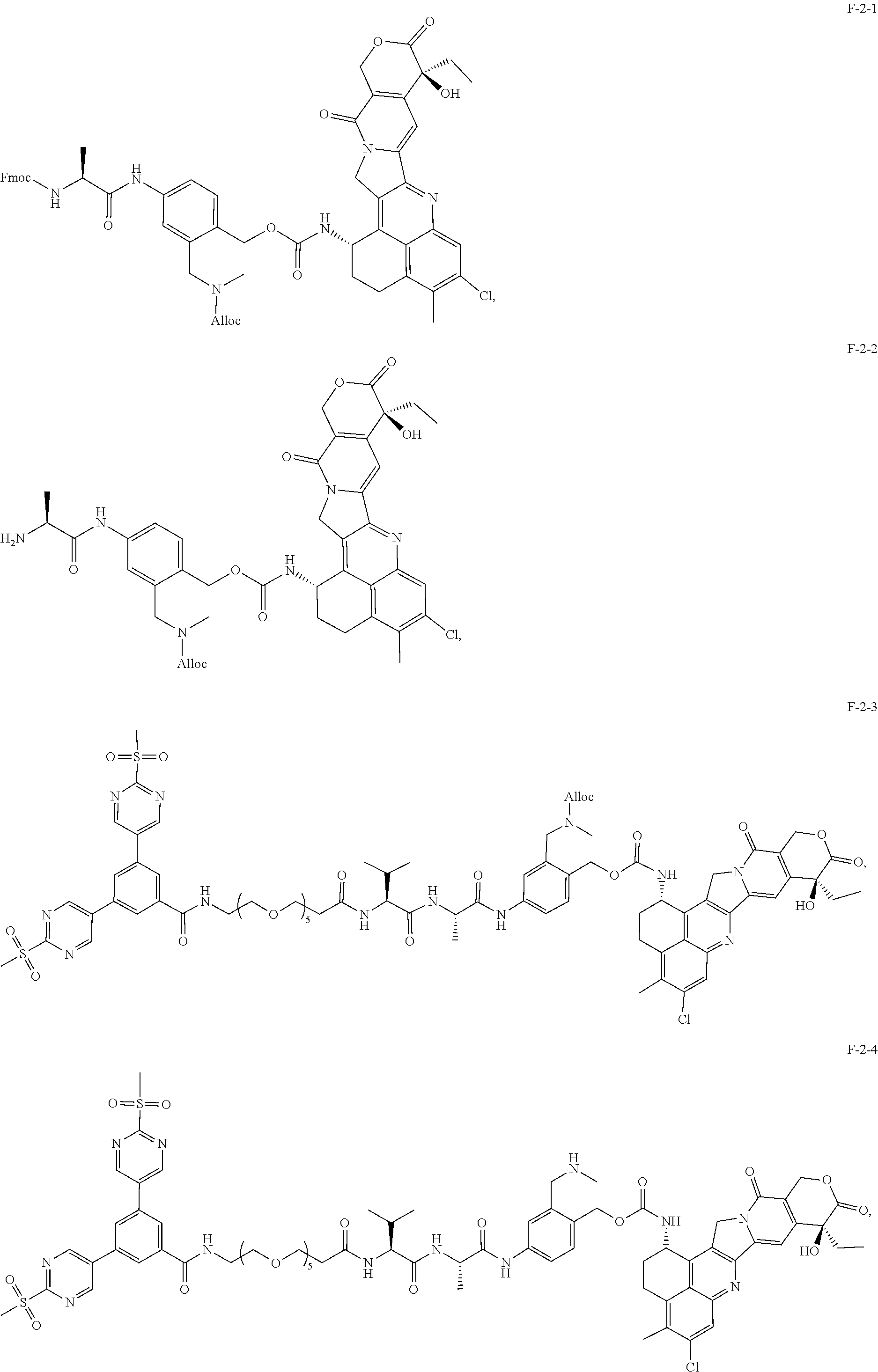

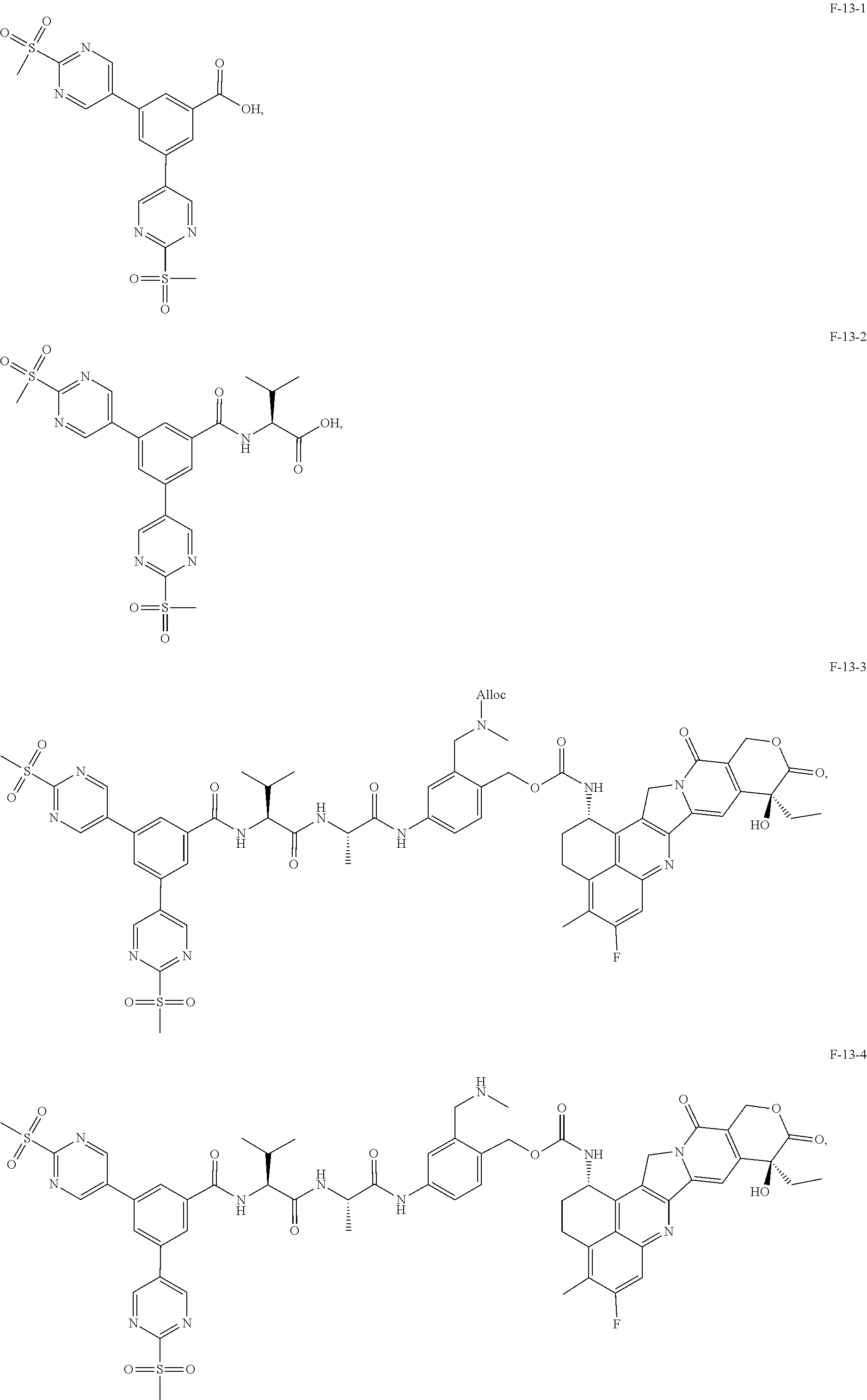

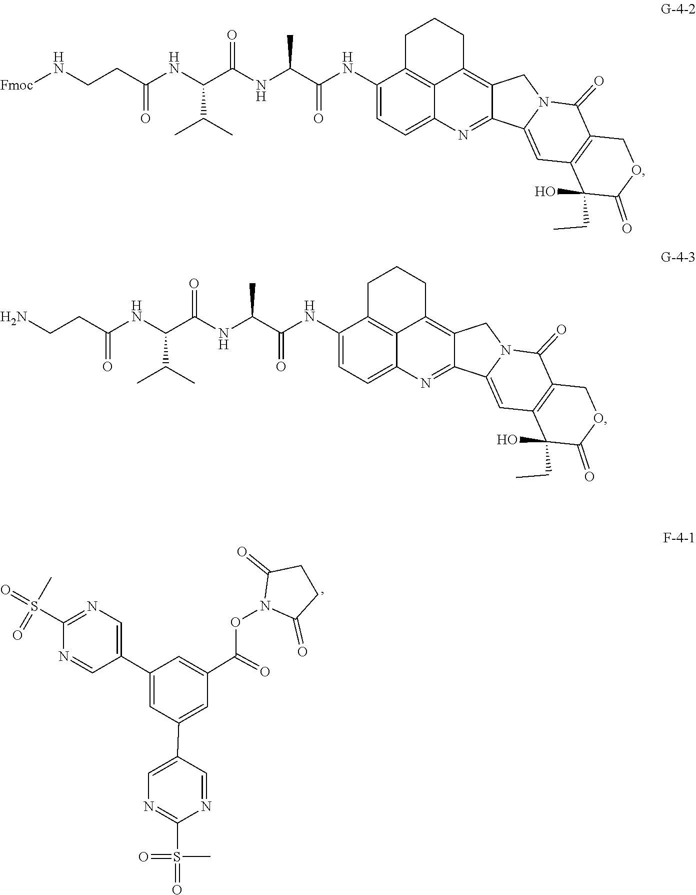

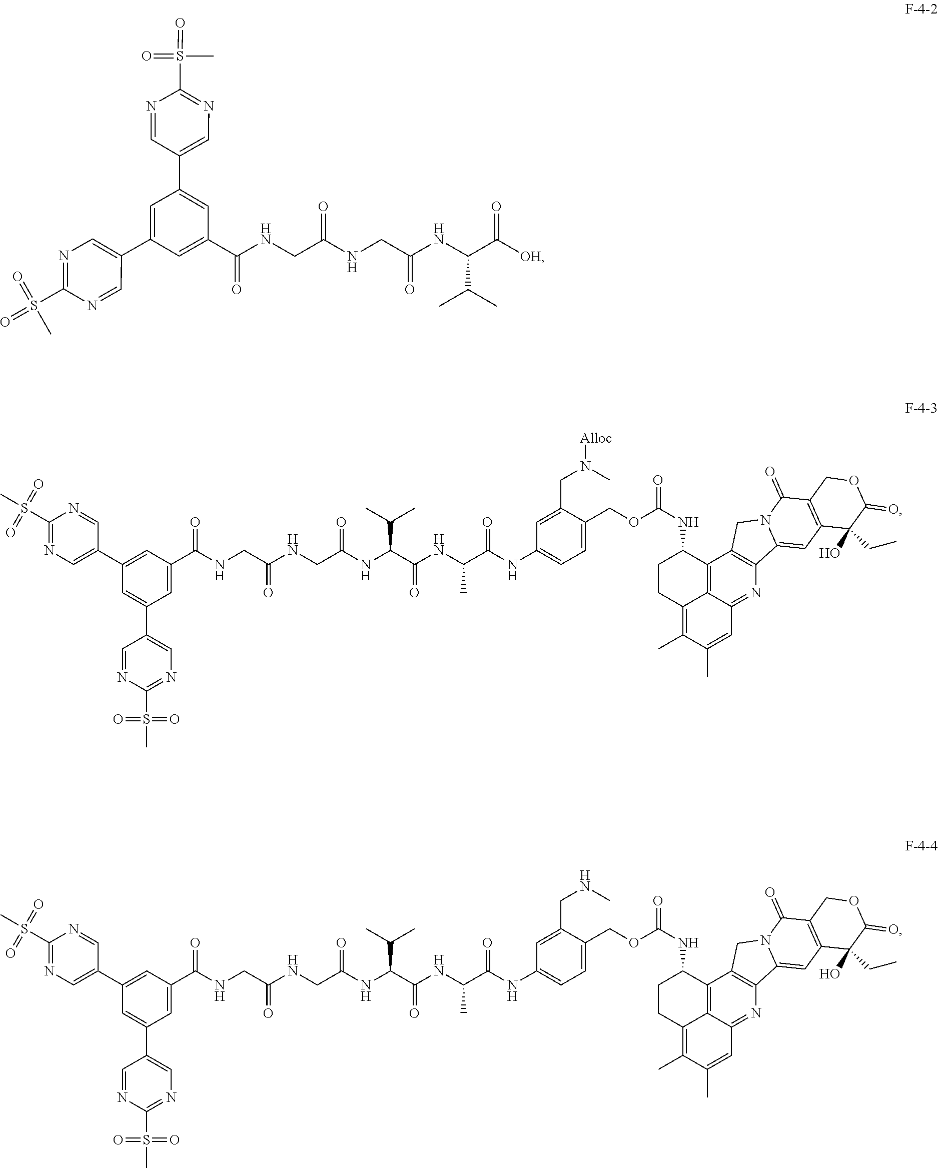

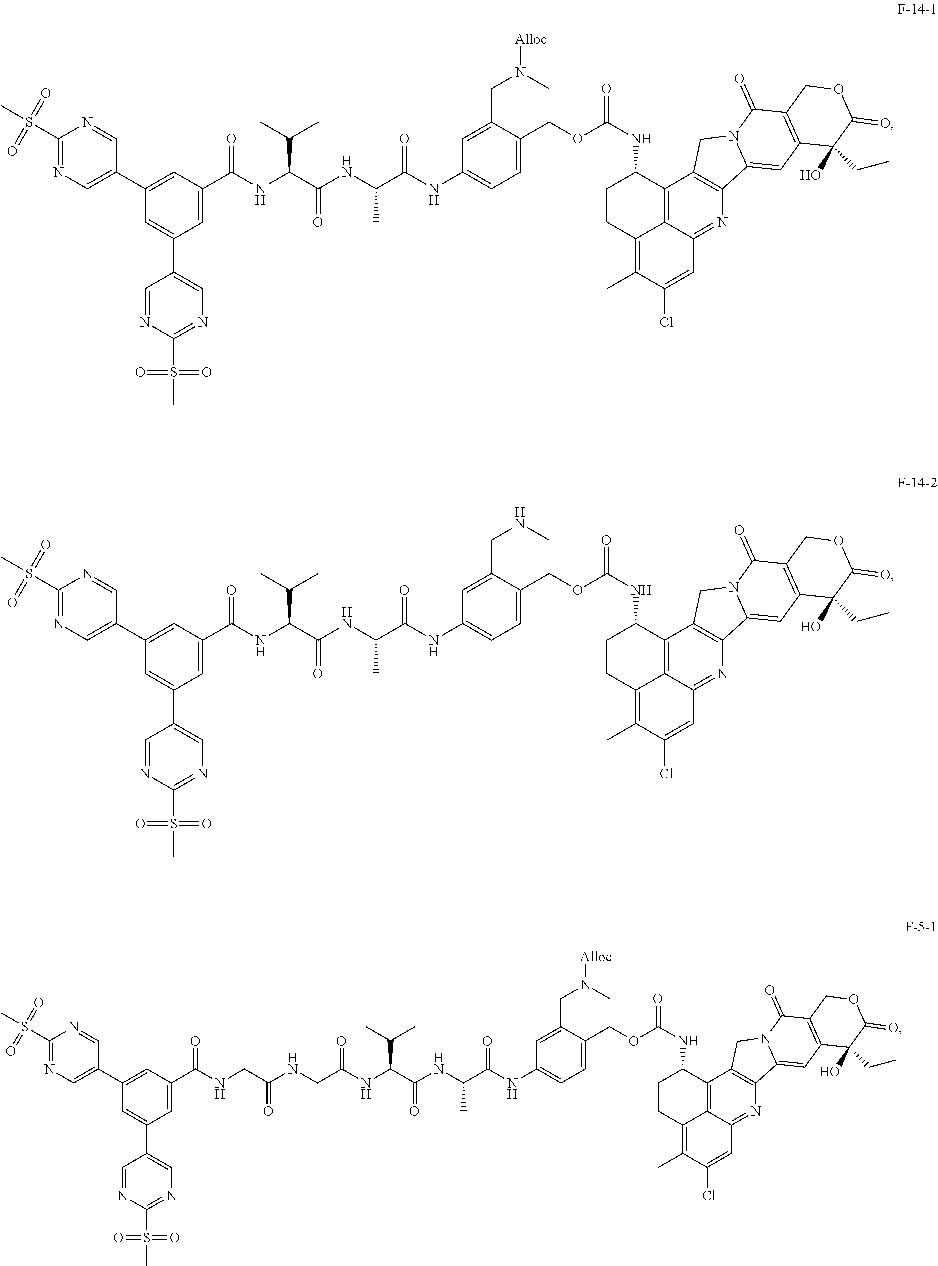

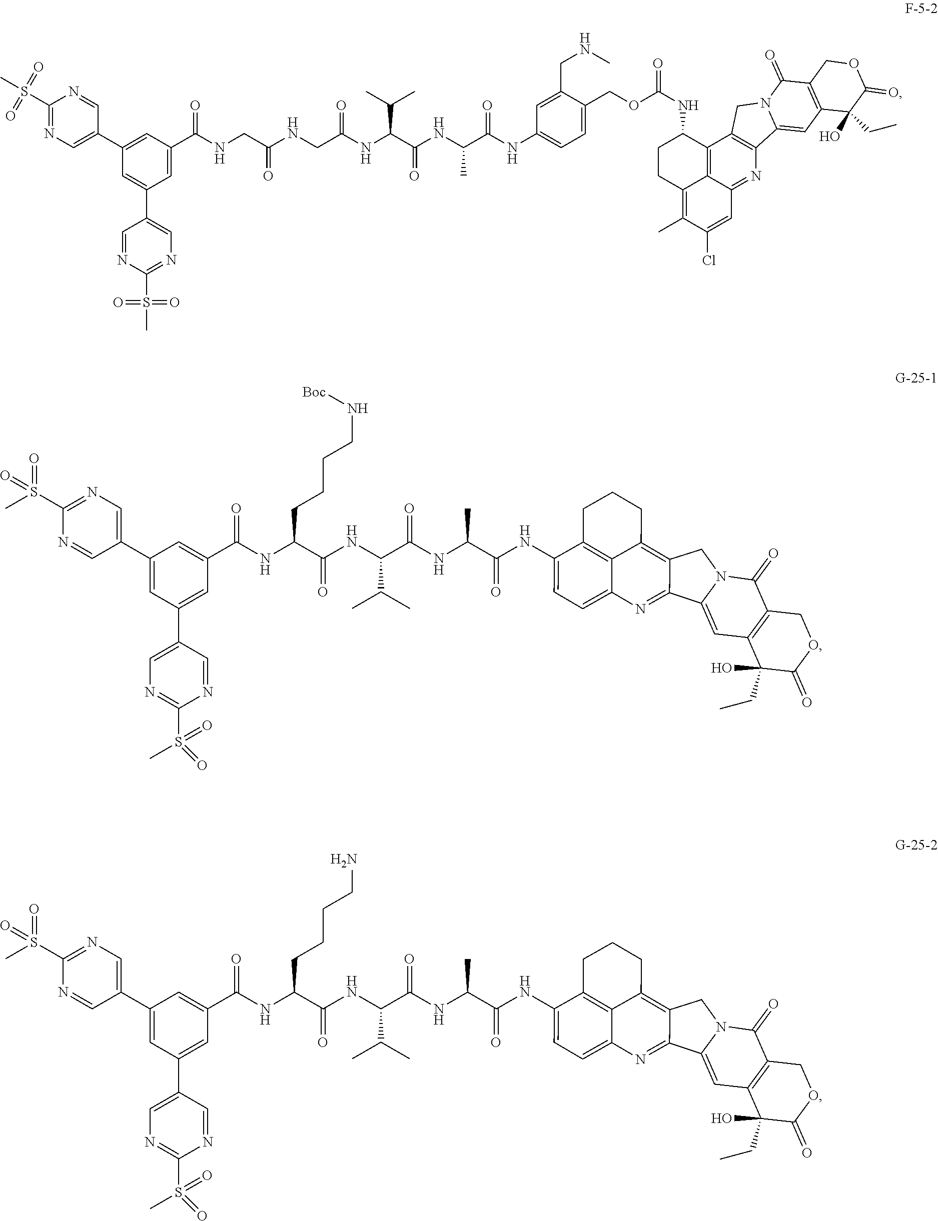

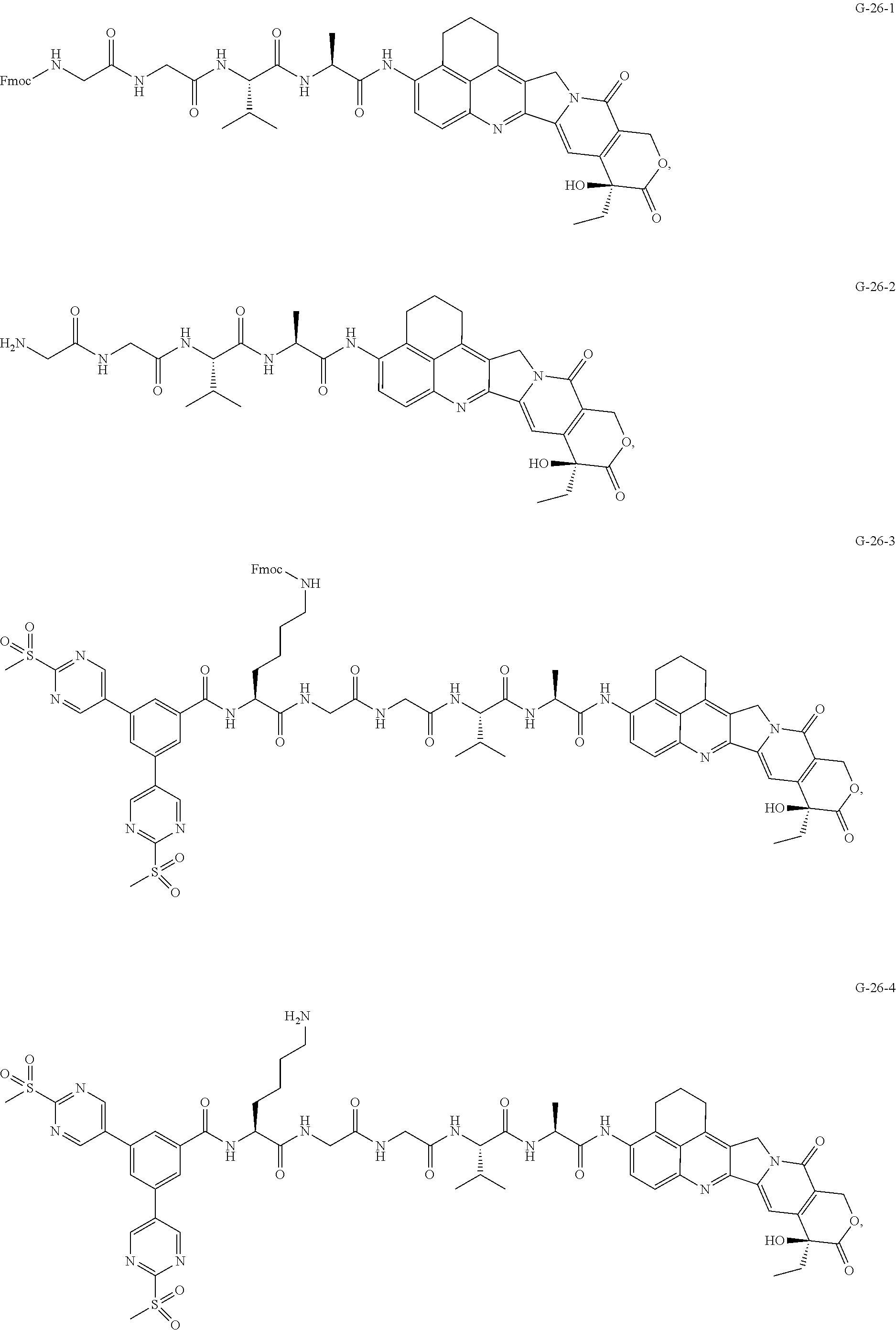

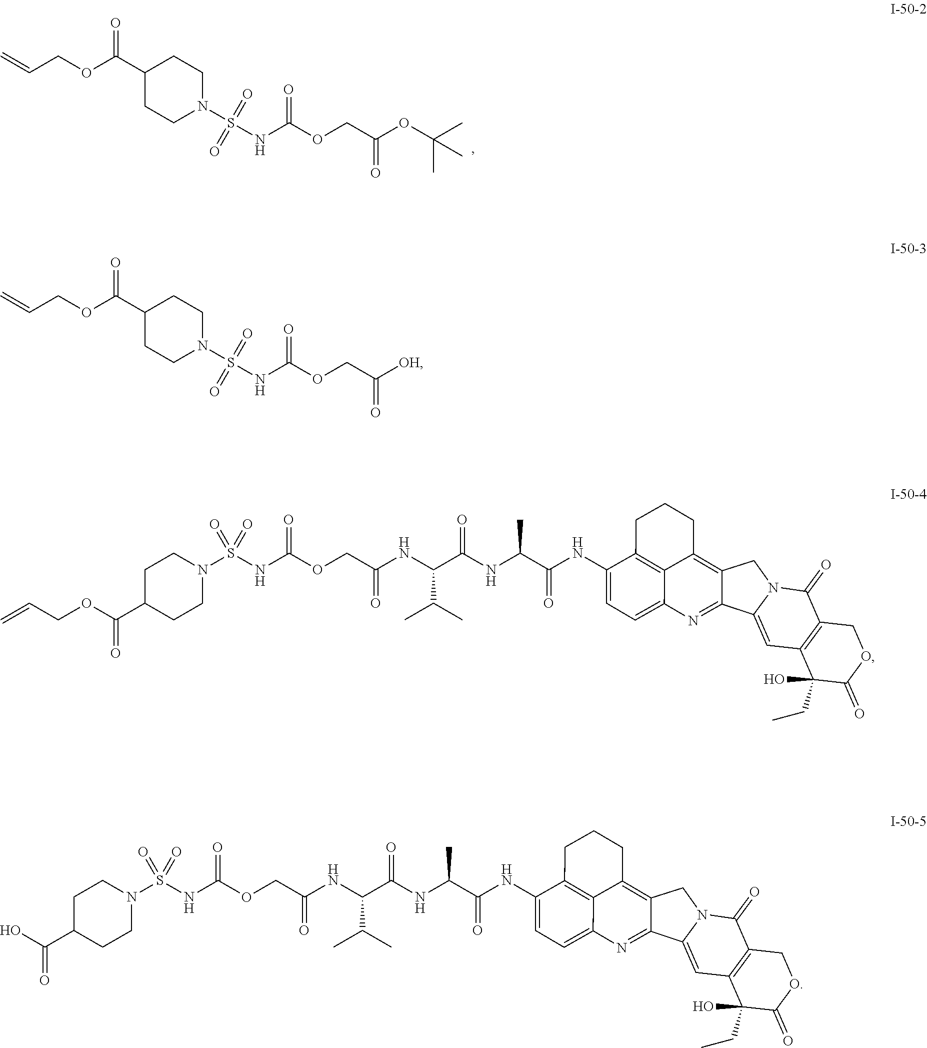 [SMILES: C=CCOC(=O)C1CCN(S(=O)(=O)NC(=O)OCC(=O)OC(C)(C)C)CC1 ; C=CCOC(=O)C1CCN(S(=O)(=O)NC(=O)OCC(=O)O)CC1 ; C=CCOC(=O)C1CCN(S(=O)(=O)NC(=O)OCC(=O)N[C@H](C(=O)N[C@@H](C)C(=O)Nc2ccc3nc4c(c5c3c2CCC5)Cn2c-4cc3c(c2=O)COC(=O)[C@]3(O)CC)C(C)C)CC1 ; CC[C@@]1(O)C(=O)OCc2c1cc1n(c2=O)Cc2c-1nc1ccc(NC(=O)[C@H](C)NC(=O)[C@@H](NC(=O)COC(=O)NS(=O)(=O)N3CCC(C(=O)O)CC3)C(C)C)c3c1c2CCC3]

In another aspect, the present application provides a use of the intermediate compound or salt, stereoisomer, tautomer or isotopically labeled compound thereof as described above in the manufacture of the compound or pharmaceutically acceptable salt thereof of the present invention.

### Synthesis Method

The present invention provides a method for synthesizing the following compound:

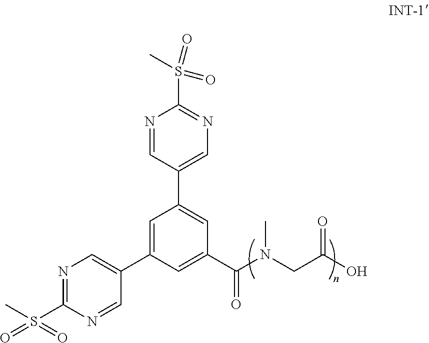

the method comprises step A of subjecting the below compound to an oxidization reaction:

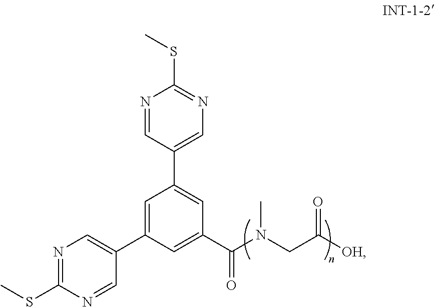

wherein n is as defined in any one of the above embodiments.

In some embodiments, the oxidization reaction is carried out in the presence of sodium periodate and/or ruthenium trichloride hydrate.

In some embodiments, n is 10.

In some embodiments, the method further comprise step B of obtaining compound INT-1-2′ by N-(((9H-fluoren-9-yl)methoxy)carbonyl)-N-methylglycine.

In some embodiments, step B is a method of solid phase synthesis.

In some embodiments, step B comprises steps of resin preparation, conjugation, deprotection, peptide cleavage and purification.

In some embodiments, the reaction materials of step B are N-(((9H-fluoren-9-yl)methoxy)carbonyl)-N-methylglycine and 3,5-bis(2-(methylthio)pyrimidin-5-yl)benzoic acid.

In some embodiments, 2-CTC resin is used in step B.

In some embodiments, the conjugation is carried out in the presence of HATU and/or DIPEA reagents.

In some embodiments, the deprotection is carried out in the presence of a piperidine reagent.

The present invention provides a method for synthesizing the following compound A-26-3′:

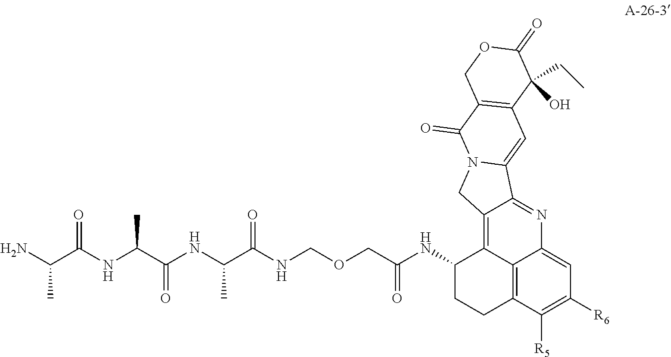

wherein R₅ and R₆ are as described in any one of the above embodiments; the method comprises a step of subjecting the following compound to a deprotection reaction:

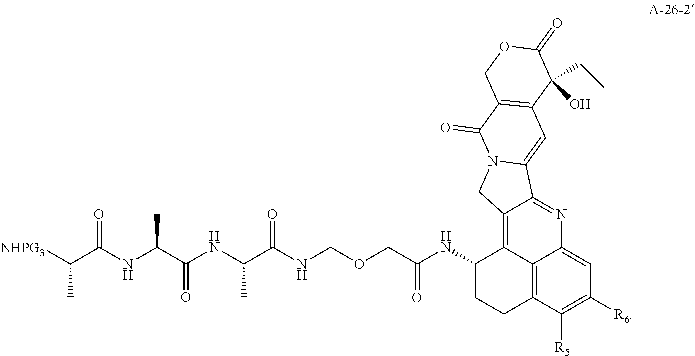

In some embodiments, the R₅ is selected from the group consisting of methyl, and R₆ is selected from the group consisting of Cl;

In some embodiments, the PG₃ is selected from the group consisting of Fmoc.

In some embodiments, the method further comprises a step of preparing compound A-26-3′ by compound 1-4′ and compound A-26-1′:

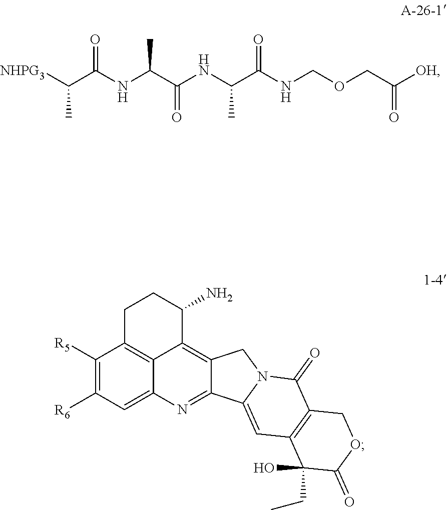

In some embodiments, the method is carried out in the presence of HATU and/or DIPEA reagent.

### Composition

In another aspect, the present application provides a composition of the antibody-drug conjugate (ADC) as described herein. Such a composition may comprise a plurality of ADCs as described herein, wherein each ADC comprises the drug-linker as described herein, wherein x is independently 1, 2, 3, 4, 5, 6, 7, 8, 9 or 10. In other words, each antibody molecule in the composition may be conjugated with 1, 2, 3, 4, 5, 6, 7, 8, 9 or 10 drug-linker. Therefore, the composition is characterized in that the “drug-to-antibody” ratio (DAR) is in the range of about 1 to about 10. Methods for determining DAR are well known to technicians, including methods using reverse phase chromatography or HPLC-MS.

For example, in any embodiment, the composition as described herein has a DAR of about 1 to about 10 or any subrange therebetween, for example, about 1, about 2, about 3, about 4, about 5, about 6, about 7, about 8, about 9, about 10, about 1 to 2, about 1 to 3, about 1 to 4, about 1 to 5, about 1 to 6, about 1 to 7, about 1 to 8, about 1 to 9, about 1 to 10, about 2 to 3, about 2 to 4, about 2 to 5, about 2 to 6, about 2 to 7, about 2 to 8, about 2 to 9, about 2 to 10, about 3 to 4, about 3 to 5, about 3 to 6, about 3 to 7, about 3 to 8, about 3 to 9, about 3 to 10, about 4 to 5, about 4 to 6, about 4 to 7, about 4 to 8, about 4 to 9, about 4 to 10, about 5 to 6, about 5 to 7, about 5 to 8, About 5 to 9, about 5 to 10, about 6 to 7, about 6 to 8, about 6 to 9, about 6 to 10, about 7 to 8, about 7 to 9, about 7 to 10, about 8 to 9, about 8 to 10, or about 9 to 10.

In certain embodiments, the DAR of the ADC composition as described herein is about 3 to 9, for example, about 3.0 to 3.5, about 3.0 to 4.0, about 3.0 to 4.5, about 3.0 to 5.0, about 3.0 to 6.0, about 3.5 to 4.0, about 3.5 to 4.5, about 3.5 to 5.0, about 3.5 to 5.5, about 3.5 to 6.0, about 3.5 to 6.5, about 4.0 to 4.5, about 4.0 to 5.0, about 4.0 to 5.5, about 4.0 to 6.0, about 4.0 to 6.5, about 4.0 to 7.0, about 4.0 to 8.0, about 4.5 to 5.0, about 4.5 to 5.5, about 4.5 to 6.0, about 4.5 to 6.5, about 4.5 to 7.0, about 4.5 to 7.5 About 5.0 to 8.0, about 5.5 to 6.0, about 5.5 to 6.5, about 5.5 to 7.0, about 5.5 to 7.5, about 5.5 to 8.0, about 6.0 to 6.5, about 6.0 to 7.0, about 6.0 to 7.5, about 6.0 to 8.5, about 6.5 to 7.0, about 6.5 to 7.5, about 6.5 to 8.5, about 7.0 to 7.5.

In certain embodiments, the DAR of the ADC composition as described herein is about 3.5 to 5.0, such as about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0.

In certain embodiments, the DAR of the ADC composition as described herein is about 1.0 to 6.0, for example, about 1.0 to 5.5, about 1.0 to 5.0, about 1.5 to 6.0, about 1.5 to about 5.5, about 1.5 to 5.0, 2.0 to 5.5, about 2.0 to about 5.0, for example, about 2.0, about 2.01, about 2.02, about 2.03, about 2.04, about 2.05, about 2.06, about 2.07, about 2.08, about 2.09, about 2.1, about 2.11, about 2.12, about 2.13, about 2.14, about 2.15, about 2.16, about 2.17, about 2.18, about 2.19, about 2.2, about 2.21, about 2.22, about 2.23, about 2.24, about 2.25, about 2.26, about 2.27, about 2.28, about 2.29, about 2.3, about 2.31, about 2.32, about 2.33, about 2.34, about 2.35, about 2.36, about 2.37, about 2.38, about 2.39, about 2.4, about 2.41, about 2.42, about 2.43, about 2.44, about 2.45, about 2.46, about 2.47, about 2.48, about 2.49, about 2.5, about 2.51, about 2.52, about 2.53, about 2.54, about 2.55, about 2.56, about 2.57, about 2.58, about 2.59, about 2.6, about 2.61, about 2.62, about 2.63, about 2.64, about 2.65, about 2.66, about 2.67, about 2.68, about 2.69, about 2.7, about 2.71, about 2.72, about 2.73, about 2.74, about 2.75, about 2.76, about 2.77, about 2.78, about 2.79, about 2.8, about 2.81, about 2.82, about 2.83, about 2.84, about 2.85, about 2.86, about 2.87, about 2.88, about 2.89, about 2.9, about 2.91, about 2.92, about 2.93, about 2.94, about 2.95, about 2.96, about 2.97, about 2.98, about 2.99, about 3.0, about 3.01, about 3.02, about 3.03, about 3.04, about 3.05, about 3.06, about 3.07, about 3.08, about 3.09, about 3.1, about 3.11, about 3.12, about 3.13, about 3.14, about 3.15, about 3.16, about 3.17, about 3.18, about 3.19, about 3.2, about 3.21, about 3.22, about 3.23, about 3.24, about 3.25, about 3.26, about 3.27, about 3.28, about 3.29, about 3.3, about 3.31, about 3.32, about 3.33, about 3.34, about 3.35, about 3.36, about 3.37, about 3.38, about 3.39, about 3.4, about 3.41, about 3.42, about 3.43, about 3.44, about 3.45, about 3.46, about 3.47, about 3.48, about 3.49, about 3.5, about 3.51, about 3.52, about 3.53, about 3.54, about 3.55, about 3.56, about 3.57, about 3.58, about 3.59, about 3.6, about 3.61, about 3.62, about 3.63, about 3.64, about 3.65, about 3.66, about 3.67, about 3.68, about 3.69, about 3.7, about 3.71, about 3.72, about 3.73, about 3.74, about 3.75, about 3.76, about 3.77, about 3.78, about 3.79, about 3.8, about 3.81, about 3.82, about 3.83, about 3.84, about 3.85, about 3.86, about 3.87, about 3.88, about 3.89, about 3.9, about 3.91, about 3.92, about 3.93, about 3.94, about 3.95, about 3.96, about 3.97, about 3.98, about 3.99, about 4.0, about 4.01, about 4.02, about 4.03, about 4.04, about 4.05, about 4.06, about 4.07, about 4.08, about 4.09, about 4.1, about 4.11, about 4.12, about 4.13, about 4.14, about 4.15, about 4.16, about 4.17, about 4.18, about 4.19, about 4.2, about 4.21, about 4.22, about 4.23, about 4.24, about 4.25, about 4.26, about 4.27, about 4.28, about 4.29, about 4.3, about 4.31, about 4.32, about 4.33, about 4.34, about 4.35, about 4.36, about 4.37, about 4.38, about 4.39, about 4.4, about 4.41, about 4.42, about 4.43, about 4.44, about 4.45, about 4.46, about 4.47, about 4.48, about 4.49, about 4.5, about 4.51, about 4.52, about 4.53, about 4.54, about 4.55, about 4.56, about 4.57, about 4.58, about 4.59, about 4.6, about 4.61, about 4.62, about 4.63, about 4.64, about 4.65, about 4.66, about 4.67, about 4.68, about 4.69, about 4.7, about 4.71, about 4.72, about 4.73, about 4.74, about 4.75, about 4.76, about 4.77, about 4.78, about 4.79, about 4.8, about 4.81, about 4.82, about 4.83, about 4.84, about 4.85, about 4.86, about 4.87, about 4.88, about 4.89, about 4.9, about 4.91, about 4.92, about 4.93, about 4.94, about 4.95, about 4.96, about 4.97, about 4.98, about 4.99, about 5.0.

In certain embodiments, the DAR of the ADC composition as described herein is about 4.41, about 4.34, about 4.51, about 4.36, about 4.63, about 4.28, about 4.38, about 3.86, about 4.16, about 3.99, or about 2.45.

In certain embodiments, the DAR of the ADC composition as described herein is 4.41, 4.34, 4.51, 4.36, 4.63, 4.28, 4.38, 3.86, 4.16, 3.99 or 2.45.

### Pharmaceutical Composition

In another aspect, the present application provides a pharmaceutical composition, which comprises the antibody-drug conjugate as described in any one of the foregoing embodiments or the composition as described in any one of the foregoing embodiments, and one or more pharmaceutical excipients.

The compound or conjugate as described herein is usually formulated together with a pharmaceutically acceptable parenteral vehicle into a unit injectable form such as bolus injection, intravenous injection, intratumoral injection for parenteral use. Optionally, the antibody-drug conjugate in the form of lyophilized agent or solution with the desired purity is mixed with a pharmaceutically acceptable diluent, carrier, excipient or stabilizer (Remington's Pharmaceutical Sciences (1980) 16^{th} edition, Osol, A. Ed.). The antibody-drug conjugate described herein or the pharmaceutical composition containing the antibody-drug conjugate can be administered via any route suitable for an individual to be treated.

### Use

The antibody-drug conjugate, composition, or pharmaceutical composition thereof described herein can be used for treating a variety of diseases or conditions, such as Her2-expressing cancer, including solid tumors or hematological malignancies, such as urothelial carcinoma, gastric cancer, breast cancer, lung cancer (e.g., non-small cell lung cancer, specifically lung adenocarcinoma), or lymphoma.

Therefore, the present application provides a use of the antibody-drug conjugate, composition, or pharmaceutical composition containing the same as described in any one of the foregoing embodiments in the manufacture of a medicament for treating Her2-expressing cancer.

At the same time, the present application provides the antibody-drug conjugate, composition, or pharmaceutical composition containing the same as described in any one of the foregoing embodiments, for use in treating Her2-expressing cancer.

At the same time, the present application also provides a method for treating Her2-expressing cancer, which comprises a step of administering to a subject in need thereof an effective amount of the antibody-drug conjugate, composition, or pharmaceutical composition containing the same as described in any one of the foregoing embodiments.

In some embodiments, the antibody-drug conjugate, composition or pharmaceutical composition containing the same is sufficient (e.g., in a subject) to:
(1) inhibit the proliferation of cells (e.g., tumor cells);
(2) inhibit tumor growth;
(3) induce and/or increase an antibody-dependent cellular cytotoxicity activity;
(4) inhibit signal transduction;
(5) prevent and/or treat a cancer; or
(6) any combination of the above (1) to (5).

In some embodiments, the cancer disease is selected from the group consisting of solid tumors or hematological malignancies, for example, gastric cancer, breast cancer, lung cancer (e.g., non-small cell lung cancer, such as lung adenocarcinoma) or urothelial carcinoma.

For all technical features disclosed herein, such as the definitions of groups, etc., except for mutually exclusive technical features, all embodiments thereof can be combined in any way to obtain different general formula ranges or specific schemes. These ranges and schemes are all within the scope of the present invention.

### Definition

Unless otherwise defined below, the meanings of all technical and scientific terms used herein are intended to be the same as those generally understood by a skilled person in the art. References to the technology used herein is shall mean the technology generally recognized in the art, including technological modifications or equivalent substitutions apparent to those skilled in the art. In addition, the laboratory procedures in genomics, nucleic acid chemistry, molecular biology, etc. used herein are all routine steps widely adopted in the respective fields. Although it is believed that the following terms are well understood by those skilled in the art, the following definitions are still provided herein to better explain the present invention.

The term “antibody” refers to an immunoglobulin molecule that is typically composed of two pairs of polypeptide chains, each pair having one light chain (LC) and one heavy chain (HC).

Antibody light chains can be categorized into κ (kappa) and λ (lambda) types. Heavy chains may be categorized into μ, δ, γ, α or ε, with corresponding antibody isotypes defined as IgM, IgD, IgG, IgA and IgE, respectively. In the light chain and heavy chain, the variable region and the constant region are connected by a “J” region of about 12 or more amino acids, and the heavy chain also contains a “D” region of about 3 or more amino acids. Each heavy chain consists of a heavy chain variable region (V_{H}) and a heavy chain constant region (CH). The heavy chain constant region consists of 3 domains (CHI, CH₂ and CH₃). Each light chain consists of a light chain variable region (V_{L}) and a light chain constant region (CL). The light chain constant region consists of one domain CL. The constant domain does not directly participate in the binding of antibodies to antigens, but exhibits a variety of effector functions, such as mediating the binding of immunoglobulins to host tissues or factors, including various cells of the immune system (e.g., effector cells) and the first component (C1q) of the classical complement system. The V_{H} and V_{L} regions may also be subdivided into highly variable regions (called complementarity determining regions (CDRs)), interspersed with more conserved regions called framework regions (FRs). Each of V_{H} and V_{L} consists of three CDRs and four FRs arranged from the amino terminus to the carboxyl terminus in the following order: FR1, CDR1, FR2, CDR2, FR3, CDR3, FR4. The variable regions (V_{H} and V_{L}) of each heavy chain/light chain pair form antigen binding sites, respectively. Amino acid numbering in each region or domain may follow various numbering systems known in the art.

The term “antibody” further includes variants where the heavy chain constant region has a C-terminal lysine, lacks a C-terminal lysine, or lacks a C-terminal glycine-lysine dipeptide. It also includes variants where the N-terminal amino acid of the antibody variable region is cyclized to pyroglutamate. Thus, in a composition comprising the disclosed antibodies, each antibody may independently have a C-terminal lysine, lack a C-terminal lysine or C-terminal glycine-lysine, and/or have a N-terminal glutamine or glutamic acid, or pyroglutamate resulting from cyclization of the N-terminal amino acid.

The term “complementarity determining region” or “CDR” refers to the amino acid residues in the variable region of an antibody that are responsible for antigen binding. The variable regions of the heavy chain and light chain each contains three CDRs, designated CDR1, CDR2, and CDR3. The precise boundaries of these CDRs may be defined according to various numbering systems known in the art, for example according to the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883), the IMGT numbering system (Lefranc et al., Dev. Comparat. Immunol. 27:55-77, 2003), or the AbM numbering system (Martin A C R, Cheetham J C, Rees A R (1989) Modelling antibody hypervariable loops: A combined algorithm. Proc Natl Acad Sci USA 86:9268-9272). For a given antibody, a person skilled in the art will readily identify the CDRs defined by each numbering system. Furthermore, the correspondence between different numbering systems is well known to a person skilled in the art (e.g., see Lefranc et al., Dev. Comparat. Immunol. 27:55-77, 2003).

In the present invention, the CDRs comprised in an antibody or antigen-binding fragment thereof may be determined according to various numbering systems known in the art, such as by KLabat, Chothia, IMGT or AbM numbering systems. In certain embodiments, the CDRs comprised in an antibody or antigen-binding fragment thereof are determined by the Chothia numbering system.

The following general rules disclosed in www.bioinf.org.uk: Professor Andrew C. R. Martin's research group may be used to define CDRs in an antibody sequence that includes those amino acids that specifically interact with the amino acids comprising the epitope in the antigen to which the antibody binds. There are rare examples where these generally constant features do not occur; however, the Cys residues are the most conserved features.

| Loop | Kabat | AbM | Chothia ¹ | Contact ² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat numbering | | | | |
| | system)³ | | | | |
| H1 | H31--H35 | H26--H35 | H26--H32 | H30--H35 | H26--H33 |
| | (Chothia numbering | | | | |
| | system) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹ Some of these numbering systems (particularly for Chothia numbering system) vary depending on the individual publication examined.
² Any of the numbering system can be used for these CDR definitions, except the Contact numbering system uses the Chothia or Martin (Enhanced Chothia) definition.
³The end of the Chothia CDR-H1 loop when numbered using the Kabat numbering system varies between H32 and H34 depending on the length of the loop. (This is because the Kabat numbering system places insertions at H35A and H35B).
If neither H35A nor H35B is present, the loop ends at H32;
If only H35A is present, the loop ends at H33;
If both H35A and H35B are present, the loop ends at H34.

The entire amino acid sequence of V_{H} is commonly numbered according to Kabat, while the three CDRs within the variable region may be defined according to any of the aforementioned numbering systems. In particular embodiments, the numbering of the amino acid positions in V_{H} may be sequential beginning with amino acid position 1 continuing sequentially to the end of the sequence or according to Kabat. Unless specified otherwise, the amino acid positions in V_{H} and V_{L} described herein are defined according to sequential numbering.

The numbering of amino acid positions in the heavy chain constant region may be sequential beginning with amino acid position 1 and continuing sequentially to the end of the sequence or according to Eu numbering. The IgG1 heavy chain constant region amino acid sequence has 330 amino acids sequentially numbered 1 to 330. The corresponding sequence numbered according to Eu begin with position number 118 and ends with position number 447. Unless specified otherwise, the amino acid positions in the heavy and light chains described herein are defined according to sequential numbering.

The term “framework region” or “FR” residues refers to those amino acid residues in the antibody variable region other than the CDR residues as defined above.

The term “antibody” is not limited to any particular method of producing the antibody. This includes, for example, recombinant antibodies, monoclonal antibodies, and polyclonal antibodies. The antibodies may be of different isotypes, for example, IgG (e.g., IgG1, IgG2, IgG3 or IgG4 subtypes), IgAQ1, IgA2, IgD, IgE or IgM antibodies.

The term “antigen-binding fragment” of an antibody refers to a polypeptide of a fragment of an antibody, such as a polypeptide of a fragment of a full-length antibody, which retains the ability to specifically bind to the same antigen bound by the full-length antibody and/or competes with the full-length antibody for specific binding to the antigen, which is also referred to as an “antigen-binding portion”. See generally, Fundamental Immunology, Ch. 7 (Paul, W., ed., 2nd ed., Raven Press, N.Y. (1989), which is incorporated herein by reference in its entirety for all purposes. An antigen-binding fragment of an antibody may be produced by a recombinant DNA technique or by an enzymatic or chemical cleavage of the intact antibody. Non-limiting examples of antigen-binding fragment include Fab fragment, Fab′ fragment, F(ab)′₂ fragment, F(ab)′₃ fragment, Fd, Fv, scFv, di-scFv, (scFv)₂, disulfide-stabilized Fv protein (“dsFv”), single domain antibody (sdAb, nanobody), and a polypeptide that comprises at least a portion of an antibody sufficient to confer specific antigen binding ability to the polypeptide. Engineered antibody variants are reviewed in Holliger et al., 2005; Nat Biotechnol, 23: 1126-1136.

The term “Fd” refers to an antibody fragment consisting of the V_{H} and CHI domains; the term “dAb fragment” refers to an antibody fragment consisting of the V_{H} domain (Ward et al., Nature 341:544 546 (1989)); the term “Fab fragment” refers to an antibody fragment consisting of the VL, VH, CL and CHI domains; the term “F(ab′)₂ fragment” refer to an antibody fragment comprising two Fab fragments connected by a disulfide bridge on the hinge region; the term “Fab′ fragment” refers to a fragment obtained after reducing the disulfide bonds connecting the two heavy chain fragments in the F(ab′)₂ fragment, which consists of a complete light chain and the Fd fragment of the heavy chain (consisting of the VH and CHI domains).

The term “Fv” refers to an antibody fragment consisting of the V_{L} and V_{H} domains of a single arm of an antibody. The Fv fragment is generally considered to be the smallest antibody fragment that may form a complete antigen binding site. It is generally believed, six CDRs confer antigen-binding specificity to an antibody. However, even a single variable region (e.g., an Fd fragment, which contains only three CDRs specific for an antigen) is able to recognize and bind to an antigen, although its affinity may be lower than that of the complete binding site.

The term “Fc” refers to an antibody fragment formed by disulfide bonding of the second and third constant regions of the first heavy chain of an antibody to the second and third constant regions of the second heavy chain. The Fc fragment of an antibody has a variety of different functions but is not involved in antigen binding.

The term “scFv” refers to a single polypeptide chain containing V_{L} and V_{H} domains connected by a linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, Roseburg and Moore, eds., Springer-Verlag, New York, pp. 269-315 (1994)). Such scFv molecules may have a general structure: NH₂-VL-linker-VH—COOH or NH₂—VH-linker-VL-COOH. A suitable prior art peptide linker consists of the repeated GGGGS (SEQ ID NO: 46) amino acid sequence or its variants. For example, a linker having the amino acid sequence (GGGGS)₄ (SEQ ID NO: 47) may be used, but variants thereof may also be used (Holliger et al. (1993), Proc. Natl. Acad. Sci. USA 90: 6444-6448). Other linkers that may be used in the present invention are described by Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol. In some cases, a disulfide bond may also exist between the V_{H} and V_{L} of the scFv. In certain embodiments, the V_{H} and V_{L} domains may be positioned relative to each other in any suitable arrangement. For example, a scFv may comprise NH₂—VH—VH—COOH, NH₂-VL-VL-COOH.

The term “single-domain antibody (sdAb)” has the meaning commonly understood by those skilled in the art, which refers to an antibody fragment composed of a single monomeric variable antibody domain (e.g., a single heavy chain variable region) that retains the ability to specifically bind to the same antigen bound by a full-length antibody (Holt, L. et al., Trends in Biotechnology, 21(11):484-490, 2003). Single-domain antibody is also called nanobody.

The above-mentioned antibody fragments all retain the ability to specifically bind to the same antigen bound by a full-length antibody, and/or compete with the full-length antibody for specific binding to the antigen.

As used therein, unless the context clearly indicates otherwise, when referring to the term “antibody”, it includes not only an intact antibody but also an antigen-binding fragment of the antibody.

An antigen-binding fragment of antibody (e.g., the above-mentioned antibody fragment) may be obtained from a given antibody (e.g., the antibody provided by the present invention) using a conventional technique known to a skilled person in the art (e.g., recombinant DNA technology or enzymatic or chemical cleavage method), and the antigen-binding fragment of antibody may be screened for specificity in the same manner as for the intact antibody.

The term “murine antibody” refers to an antibody obtained by the following method: fusing B cells of immunized mice with myeloma cells, screening for murine hybrid fusion cells that may both proliferate indefinitely and secrete antibodies, followed by screening, antibody preparation and antibody purification; or refers to an antibody secreted by plasma cells formed by differentiation and proliferation of B cells after an antigen invades mice.

The term “humanized antibody” refers to a non-human antibody modified by genetic engineering, of which amino acid sequence is modified to increase the homology with the sequence of a human antibody. Generally speaking, all or part of the CDR region of a humanized antibody comes from a non-human antibody (donor antibody), and all or part of the non-CDR region (e.g., variable region FR and/or constant region) comes from a human immunoglobulin (recipient antibody). The humanized antibody generally retains the expected properties of the donor antibody, including, but not limited to, antigen specificity, affinity, reactivity, ability to increase immune cell activity, ability to enhance immune response, etc. The donor antibody may be a mouse, rat, rabbit or non-human primate (e.g., cynomolgus monkey) antibody with the expected properties (e.g., antigen specificity, affinity, reactivity, ability to increase immune cell activity and/or ability to enhance immune response).

The term “identity” is used to refer to a match of sequences between two polypeptides or between two nucleic acids. When a position in both sequences being compared is occupied by the same base or amino acid monomer subunit (e.g., a position in each of two DNA molecules is occupied by adenine, or a position in each of two polypeptides is occupied by lysine), then the molecules are identical at that position. “Percent identity” between two sequences is a function of the number of matching positions common to the two sequences divided by the number of positions compared×100. For example, if 6 out of 10 positions of two sequences match, then the two sequences are 60% identical. For example, the DNA sequences CTGACT and CAGGTT share 50% identity (3 positions match out of 6 total positions). Typically, comparisons are made when two sequences are aligned to yield maximum identity. Such alignments can be achieved using, for example, the method of Needleman et al. (1970) J. Mol. Biol. 48: 443-453, which can be conveniently performed by computer programs such as the Align program (DNAstar, Inc.). The algorithm of E. Meyers and W. Miller (Comput. Appl Biosci., 4:11-17 (1988)) which has been incorporated into the ALIGN program (version 2.0), can be used to determine the percent identity between two amino acid sequences by using the PAM120 weight residue table, a gap length penalty of 12, and a gap penalty of 4. Alternatively, the algorithm of Needleman and Wunsch (J MoI Biol. 48:444-453 (1970)) which has been integrated into the GAP program of the GCG software package (available at www.gcg.com) can be used to determine the percent identity between two amino acid sequences by using the Blossum 62 matrix or PAM250 matrix with a gap weight of 16, 14, 12, 10, 8, 6, or 4, and a length weight of 1, 2, 3, 4, 5, or 6.

The term “conservative substitution” refers to an amino acid substitution that does not adversely affect or change the expected properties of the protein/polypeptide comprising the amino acid sequence. For example, a conservative substitution may be introduced by a standard technique known in the art such as site-directed mutagenesis and PCR-mediated mutagenesis. Conservative amino acid substitutions include substitutions in which amino acid residues are substituted with amino acid residues having similar side chains, such as substitutions with residues that are physically or functionally similar to the corresponding amino acid residues (e.g., having similar size, shape, charge, chemical properties, including the ability to form covalent bonds or hydrogen bonds, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), P-branched side chains (e.g., threonine, valine, isoleucine), and aromatic side chains (e.g., tyrosine, phenylalanine, tryptophan, histidine). Thus, it is preferred to replace a corresponding amino acid residue with another amino acid residue from the same side chain family. Methods for identifying conservative substitutions of amino acids are well known in the art (see, for example, Brummell et al., Biochem. 32:1180-1187 (1993); Kobayashi et al. Protein Eng. 12(10):879-884 (1999); and Burks et al. Proc. Natl Acad. Set USA 94:412-417 (1997), which are incorporated herein by reference).

The twenty conventional amino acids used herein are written in accordance with conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, amino acids are generally represented by single-letter and three-letter abbreviations known in the art. For example, alanine may be represented by A or Ala.

The term “linker” refers to a structural fragment that connects a cytotoxic drug to an antibody or antigen-binding fragment thereof. For example, it refers to the -M-L-E- structural fragment in the formula Ab-[M-L-E-D]ₓ.

The term “drug-linker” refers to a structure of the cytotoxic drug and linker described in the present invention before being connected to an antibody or antigen-binding fragment thereof. For example, “drug-linker” refers to M′-L-E-D, wherein M′ is the structural form of M before covalently connecting to an antibody or antigen-binding fragment thereof. “Drug-linker” is covalently connected to an antibody or antigen-binding fragment thereof to obtain the antibody-drug conjugate described in the present application.

The “drug-linker” also includes all pharmaceutically acceptable isotopically labeled compounds thereof, which are the same as the “drug-linker” compound of the present invention, except that one or more atoms are replaced by atoms having the same atomic number but an atomic mass or mass number different from the atomic mass or mass number that predominates in nature. Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O, and ¹⁸O); and isotopes of sulfur (e.g., ³⁵S).

The terms “include,” “comprises,” “have,” “contain,” or “involve,” and other variations thereof as used herein, are inclusive or open-ended and do not exclude other unlisted elements or method steps.

The term “alkyl” refers to a group, such as “C₁₋₂₀ alkyl”, “C₁₋₁₀ alkyl”, “C₁₋₆ alkyl”, “C₁₋₄ alkyl”, “C₁₋₃ alkyl”, etc., which is obtained by removing one hydrogen atom from a straight or branched hydrocarbonyl. Its specific examples include, but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “alkylene” refers to a group obtained by removing two hydrogen atoms from a straight or branched hydrocarbonyl group, such as “C₁₋₆ alkylene”, “C₁₋₅ alkylene”, “C₁₋₄ alkylene”, “C₁₋₃ alkylene”, and its specific examples include but are not limited to: methylene, ethylene, propylene, butylene, etc.

The term “aryl” refers to an unsaturated carbocyclic group with a conjugated π electron system, such as “6- to 10-membered aryl”, and its specific examples include but are not limited to: phenyl, naphthyl.

The term “heteroaryl” refers to an unsaturated group with a conjugated π electron system composed of ring atoms, in which at least one (e.g. 1, 2, 3 or 4) of the ring atoms is a heteroatom, such as N, O and S, wherein the nitrogen atom is optionally quaternized, and the nitrogen and sulfur heteroatoms are optionally oxidized. For example, it can be “5- to 12-membered heteroaryl”, “5- to 11-membered heteroaryl”, “5- to 10-membered heteroaryl”, “5- to 9-membered heteroaryl”, “5- to 6-membered heteroaryl”, and its specific examples include but are not limited to: phenyl, naphthyl, furanyl, pyrrolyl, thienyl, pyrazolyl, imidazolyl, thiazolyl, isothiazolyl, thiadiazolyl, oxazolyl, isoxazolyl, oxadiazolyl, pyridyl, pyrimidinyl, pyridazinyl, pyrazinyl, indazolyl, indolyl, quinolyl, isoquinolyl.

The term “heterocyclyl” refers to a saturated or partially unsaturated cyclic group composed of ring atoms, in which at least one of the ring atoms (e.g., 1, 2, 3, 4 or 5 heteroatoms) is a heteroatom, such as N, O and S, wherein the nitrogen atom is optionally quaternized, the nitrogen and sulfur heteroatoms are optionally oxidized, and the carbon atom is optionally oxidized. The heterocyclyl comprises monocyclic ring, bicyclic ring or polycyclic ring, including spirocyclic ring, fused cyclic ring or bridged cyclic ring. The term “nitrogen-containing heterocyclyl” means that at least one heteroatom is N, such as “5- to 12-membered nitrogen-containing heterocyclyl”, “5- to 9-membered nitrogen-containing heterocyclyl”, “9- to 12-membered nitrogen-containing heterocyclyl”, and its specific examples include but are not limited to: pyrrolidinyl, piperidinyl, piperazinyl, morpholinyl, 1,2,3,4-tetrahydroquinolinyl.

The term “isotopically labeled compound” means that the compound is identical in structure to the compound of the present invention except that one or more atoms are replaced by atoms having the same atomic number but an atomic mass or mass number different from the atomic mass or mass number that predominates in nature. Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O, and ¹⁸O); and isotopes of sulfur (e.g., ³⁵S).

As used herein, the term “suitable substituent” refers to a modification that a person skilled in the art can make to a compound according to the need of the compound substituent. “Suitable substituent” includes oxo (═O), halogen, cyano, NR⁸R⁹, carboxyl, sulfhydryl group, hydroxyl, ester (e.g., —C₁₋₆ alkyl-C(═O)—OC₁₋₆ alkyl), C₁₋₆ alkyl, C₂₋₆ alkenyl, C₂₋₆ alkynyl, C₁₋₆ alkyl-O—C₁₋₆ alkyl, C₁₋₆ haloalkyl, C₁₋₆ alkoxy, C₃₋₆ cycloalkyl, 3- to 10-membered heterocyclyl, 5- to 10-membered heteroaryl, C₆₋₁₀ aryl, benzyl, hydroxy-substituted benzyl, indolylmethylene and C₁₋₆ haloalkoxy, wherein R⁸, R⁹ are each independently selected from the group consisting of H, C₁₋₆ alkyl, C₃₋₆ cycloalkyl, 3- to 10-membered heterocyclyl, 5- to 10-membered heteroaryl, C₆₋₁₀ aryl, C₁₋₆ alkoxy, C₁₋₆ haloalkyl, C₁₋₆ haloalkoxy, halogen, hydroxyl, carboxyl and ester (e.g. —C₁₋₆ alkyl-C(═O)—OC₁₋₆ alkyl).

The term “substituted” means that one or more (e.g. 1, 2, 3, 4 or 5) hydrogen atoms on the specified compound or structural fragment are replaced by a substituent, provided that the normal valence of the specified atom in the current situation is not exceeded and the substitution forms a stable compound. Combinations of substituents and/or variables are permissible only if they yield stable compounds. In some embodiments, the substituent is each independently composed of one or more of the following structures: —O—, —S—, —NR′—, halogen, —CN, —OH, —NH₂, —NO₂, —CN, ═O, C₁-C₆ alkyl(ene), C₁-C₆ haloalkyl(ene), C₁-C₆ alkoxy, C₂-C₆ alkenyl(ene), C₂-C₆ alkynyl(ene), C₃-C₈ cycloalkyl(ene), 3- to 8-membered heterocyclyl(ene), C₆-C₁₀ aryl(ene) and 5- to 10-membered heteroaryl(ene), etc. In some embodiments, the substituent is each independently composed of one or more of the following structures: NR⁸R⁹, —O—, —S—, —NR′—, halogen, —CN, —OH, —SH, —NH₂, —NO₂, —C(O)—, —CN, ═O, C₁-C₆ alkyl(ene), C₁-C₆ haloalkyl(ene), C₁-C₆ alkoxy, C₂-C₆ alkenyl(ene), C₂-C₆alkynyl(ene), C₃-C₈ cycloalkyl(ene), 3- to 10-membered heterocyclyl(ene), C₆-C₁₀ aryl(ene), and 5- to 10-membered heteroaryl(ene), etc., wherein R⁸, R⁹ and R′ are as defined above. For example, the substituents may be suitable substituents as described above.

If a functional group or structural fragment is described as “substituted or unsubstituted”, the functional group or structural fragment may be (1) unsubstituted or (2) substituted.

Whether or not explicitly indicated, all numerical values in the present application are modified by the term “about”. The term “about” refers to a range of ±20% of the stated value, ±10% of the stated value, preferably ±5% of the stated value, and more preferably ±2% of the stated value.

### BRIEF DESCRIPTION OF THE DRAWINGS

FIG. 1 shows the efficacy results of anti-human HER2 antibody-drug conjugate ADCs on NCI-N87 cell subcutaneous tumor-bearing mouse model.

FIG. 2 shows the changes in body weight of the mice of each group in the human gastric cancer cell NCI-N87 CDX model.

FIG. 3 shows the efficacy results of anti-human HER2 antibody-drug conjugate ADCs on NCI-N87 cell subcutaneous tumor-bearing mouse model.

FIG. 4 shows the efficacy results of anti-human HER2 antibody-drug conjugate ADCs on NCI-N87 cell subcutaneous tumor-bearing mouse model.

FIG. 5 shows the changes in body weight of the mice of each group in the human gastric cancer cell NCI-N87 CDX model.

### SPECIFIC MODELS FOR CARRYING OUT THE PRESENT INVENTION

The present invention is further described below through the description of specific embodiments, but this is not a limitation of the present invention. Based on the teachings of the present invention, those skilled in the art can make various modifications or improvements without departing from the basic concept and scope of the present invention.

The information of the sequences involved in the present invention is described in the following table:

| SEQ ID | | |
|---|---|---|
| NO: | Sequence Name | Sequence Information |
| 1 | Trastuzumab VH | EVQLVESGGGLVQPGGSLRLSCAASGFNIKDTYIHWVRQAPGKGLE |
| | amino acid | WVARIYPTNGYTRYADSVKGRFTISADTSKNTAYLQMNSLRAEDTA |
| | sequence | VYYCSRWGGDGFYAMDYWGQGTLVTVSS |
| 2 | Trastuzumab VL | DIQMTQSPSSLSASVGDRVTITCRASQDVNTAVAWYQQKPGKAPKL |
| | amino acid | LIYSASFLYSGVPSRFSGSRSGTDFTLTISSLQPEDFATYYCQQHYTTP |
| | sequence | PTFGQGTKVEIK |
| | Pertuzumab VH | EVQLVESGGGLVQPGGSLRLSCAASGFTFTDYTMDWVRQAPGKGL |
| 3 | amino acid | EWVADVNPNSGGSIYNQRFKGRFTLSVDRSKNTLYLQMNSLRAED |
| | sequence | TAVYYCARNLGPSFYFDYWGQGTLVTVSS |
| 4 | Pertuzumab VL | DIQMTQSPSSLSASVGDRVTITCKASQDVSIGVAWYQQKPGKAPKL |
| | amino acid | LIYSASYRYTGVPSRFSGSGSGTDFTLTISSLQPEDFATYYCQQYYIY |
| | sequence | PYTFGQGTKVEIK |
| 5 | Trastuzumab | GFNIKDT |
| | Chothia CDR H1 | |
| 6 | Trastuzumab | YPTNGY |
| | Chothia CDR H2 | |
| 7 | Trastuzumab | WGGDGFYAMDY |
| | Chothia/kabat/AbM | |
| | CDR H3 | |
| 8 | Trastuzumab | RASQDVNTAVA |
| | Chothia/Kabat/AbM | |
| | CDR L1 | |
| 9 | Trastuzumab | SASFLYS |
| | Chothia/Kabat/AbM | |
| | CDR L2 | |
| 10 | Chothia/Kabat/ | QQHYTTPPT |
| | IMGT/AbM CDR L3 | |
| 11 | Trastuzumab | DTYIH |
| | Kabat CDR H1 | |
| 12 | Trastuzumab | RIYPTNGYTRYADSVKG |
| | Kabat CDR H2 | |
| 13 | Trastuzumab | GFNIKDTY |
| | IMGT CDR H1 | |
| 14 | Trastuzumab | IYPTNGYT |
| | IMGT CDR H2 | |
| 15 | Trastuzumab | SRWGGDGFYAMDY |
| | IMGT CDR H3 | |
| 16 | Trastuzumab | QDVNTA |
| | IMGT CDR L1 | |
| 17 | Trastuzumab | SAS |
| | IMGT CDR L2 | |
| 18 | Trastuzumab AbM | GFNIKDTYIH |
| | CDR H1 | |
| 19 | Trastuzumab AbM | RIYPTNGYTR |
| | CDR H2 | |
| 20 | Pertuzumab | GFTFTDY |
| | Chothia CDR H1 | |
| 21 | Pertuzumab | NPNSGG |
| | Chothia CDR H2 | |
| 22 | Pertuzumab | NLGPSFYFDY |
| | Chothia/Kabat/AbM | |
| | CDR H3 | |
| 23 | Pertuzumab | KASQDVSIGVA |
| | Chothia/Kabat/AbM | |
| | CDR L1 | |
| 24 | Pertuzumab | SASYRYT |
| | Chothia/Kabat/AbM | |
| | CDR L2 | |
| 25 | Pertuzumab | QQYYIYPYT |
| | Chothia/Kabat/ | |
| | IMGT/AbM CDR L3 | |
| 26 | Pertuzumab Kabat | DYTMD |
| | CDR H1 | |
| 27 | Pertuzumab Kabat | DVNPNSGGSIYNQRFKG |
| | CDR H2 | |
| 28 | Pertuzumab IMGT | GFTFTDYT |
| | CDR H1 | |
| 29 | Pertuzumab IMGT | VNPNSGGS |
| | CDR H2 | |
| 30 | Pertuzumab IMGT | ARNLGPSFYFDY |
| | CDR H3 | |
| 31 | Pertuzumab IMGT | QDVSIG |
| | CDR L1 | |
| 32 | Pertuzumab IMGT | SAS |
| | CDR L2 | |
| 33 | Pertuzumab AbM | GFTFTDYTMD |
| | CDR H1 | |
| 34 | Pertuzumab AbM | DVNPNSGGSI |
| | CDR H2 | |
| 35 | Heavy chain | ASTKGPSVFPLAPSSKSTSGGTAALGCLVKDYFPEPVTVSWNSGALT |
| | constant region | SGVHTFPAVLQSSGLYSLSSVVTVPSSSLGTQTYICNVNHKPSNTKV |
| | amino acid | DKKVEPKSCDKTHTCPPCPAPELLGGPSVFLFPPKPKDTLMISRTPEV |
| | sequence of | TCVVVDVSHEDPEVKFNWYVDGVEVHNAKTKPREEQYNSTYRVV |
| | Trastuzumab or | SVLTVLHQDWLNGKEYKCKVSNKALPAPIEKTISKAKGQPREPQVY |
| | Pertuzumab | TLPPSREEMTKNQVSLTCLVKGFYPSDIAVEWESNGQPENNYKTTPP |
| | | VLDSDGSFFLYSKLTVDKSRWQQGNVFSCSVMHEALHNHYTQKSL |
| | | SLSPGK |
| 36 | Light chain | RTVAAPSVFIFPPSDEQLKSGTASVVCLLNNFYPREAKVQWKVDNA |
| | constant region | LQSGNSQESVTEQDSKDSTYSLSSTLTLSKADYEKHKVYACEVTHQ |
| | amino acid | GLSSPVTKSFNRGEC |
| | sequence of | |
| | Trastuzumab or | |
| | Pertuzumab | |
| 37 | Heavy chain | EVQLVESGGGLVQPGGSLRLSCAASGFNIKDTYIHWVRQAPGKGLE |
| | amino acid | WVARIYPTNGYTRYADSVKGRFTISADTSKNTAYLQMNSLRAEDTA |
| | sequence of | VYYCSRWGGDGFYAMDYWGQGTLVTVSSASTKGPSVFPLAPSSKS |
| | Trastuzumab | TSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEY |
| | | KCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVD |
| | | KSRWQQGNVFSCSVMHEALHNHYTQKSLSLSPGK |
| 38 | Light chain amino | DIQMTQSPSSLSASVGDRVTITCRASQDVNTAVAWYQQKPGKAPKL |
| | acid sequence of | LIYSASFLYSGVPSRFSGSRSGTDFTLTISSLQPEDFATYYCQQHYTTP |
| | Trastuzumab | PTFGQGTKVEIKRTVAAPSVFIFPPSDEQLKSGTASVVCLLNNFYPRE |
| | | AKVQWKVDNALQSGNSQESVTEQDSKDSTYSLSSTLTLSKADYEK |
| | | HKVYACEVTHQGLSSPVTKSFNRGEC |
| 39 | Heavy chain | EVQLVESGGGLVQPGGSLRLSCAASGFTFTDYTMDWVRQAPGKGL |
| | amino acid | EWVADVNPNSGGSIYNQRFKGRFTLSVDRSKNTLYLQMNSLRAED |
| | sequence of | TAVYYCARNLGPSFYFDYWGQGTLVTVSSASTKGPSVFPLAPSSKS |
| | Pertuzumab | TSGGTAALGCLVKDYFPEPVTVSWNSGALTSGVHTFPAVLQSSGLY |
| | | SLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEY |
| | | KCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVD |
| | | KSRWQQGNVFSCSVMHEALHNHYTQKSLSLSPGK |
| 40 | Light chain amino | DIQMTQSPSSLSASVGDRVTITCKASQDVSIGVAWYQQKPGKAPKL |
| | acid sequence of | LIYSASYRYTGVPSRFSGSGSGTDFTLTISSLQPEDFATYYCQQYYIY |
| | Pertuzumab | PYTFGQGTKVEIKRTVAAPSVFIFPPSDEQLKSGTASVVCLLNNFYPR |
| | | EAKVQWKVDNALQSGNSQESVTEQDSKDSTYSLSSTLTLSKADYEK |
| | | HKVYACEVTHQGLSSPVTKSFNRGEC |
| 41 | Short peptide | GGFG |
| 42 | Short peptide | GGVA |
| 43 | Short peptide | GFLG |
| 44 | Short peptide | EAAA |
| 45 | Short peptide | GGGGG |
| 46 | Linker subunit | GGGGS |
| 47 | (GGGGS)₄ | GGGGSGGGGSGGGGSGGGGS |

The abbreviations used herein have the following meanings:

| Abbreviation | Meaning | Abbreviation | Meaning |
|---|---|---|---|
| HATU | N,N,N′,N′-Tetramethyl-O-(7- | EDCI | 1-(3-Dimethylaminopropyl)-3- |
| | azabenzotriazol-1-yl)uronium | | ethylcarbodiimide hydrochloride |
| | hexafluorophosphate | | |
| EEDQ | 2-Ethoxy-1-ethoxycarbonyl- | HOBt | 1-hydroxybenzotriazole |
| | 1,2-dihydroquinoline | | |
| PyBOP | 1H-Benzotriazol-1- | DMTMM | 4-(4,6-Dimethoxytriazin-2-yl)- |
| | yloxytripyrrolidinyl | | 4-methylmorpholine |
| | hexafluorophosphate | | hydrochloride |
| DIPEA | Diisopropylethylamine | DBU | 1,8-Diazobispiro[5.4.0]undec-7- |
| | | | ene |
| DMAP | 4-Dimethylaminopyridine | DCC | N,N′-dicyclohexylcarbodiimide |
| HOSu | N-hydroxysuccinimide | PPTS | Pyridinium p-toluenesulfonate |
| TsOH | p-Toluenesulfonic acid | TFA | Trifluoroacetic acid |
| AcOH | Acetic acid | LiOH•H₂O | Lithium hydroxide monohydrate |
| NaHCO₃ | Sodium bicarbonate | Pd(PPh₃)₄ | Tetra(triphenylphosphine) |
| | | | palladium |
| Pb(OAc)₄ | Lead tetraacetate | MMAE | Monomethyl auristatin E |
| AE | Auristatin E | Cit | Citrulline |
| Val | Valine | Boc | tert-Butyloxycarbonyl |
| PABC | p-Aminobenzyl alcohol | Pt/C | Platinum on carbon |
| Pd/C | Palladium on carbon | HPLC | High performance liquid |
| | | | chromatography |
| LCMS | Liquid chromatography-mass | Rf | Retardation factor |
| | spectrometry | | |
| Pre-TLC | Preparative thin layer | DMSO | Dimethyl sulfoxide |
| | chromatography | | |
| PE | Petroleum ether | EA | Ethyl acetate |
| THF | Tetrahydrofuran | DMF | N,N-dimethylformamide |
| MeOH | Methanol | DCM | Dichloromethane |
| IPA | Isopropanol | MeCN | Acetonitrile |
| MTBE | Methyl tert-butyl ether | H₂O | Water |
| PB | Phosphate buffer | EDTA | Ethylenediaminetetraacetic acid |
| Na₂HPO₄ | Disodium hydrogen phosphate | TCEP | Tris(2-carbonylethyl)phosphine |
| | | | hydrochloride |
| DAR | Drug-to-antibody ratio | | |

The structures of the compounds described in the following examples were determined by nuclear magnetic resonance (¹H NMR) or mass spectrometry (MS).

Nuclear magnetic resonance (¹H NMR) was measured using a Bruker 400 MHz nuclear magnetic resonance instrument; the deuterated reagent was hexadeuterated dimethyl sulfoxide (DMSO-d6); and the internal standard substance was tetramethylsilane (TMS).

The abbreviations in the nuclear magnetic resonance (NMR) spectra used in the examples are shown below.

s: singlet, d: doublet, t: triplet, q: quartet, m: multiplet, br: broad, J: coupling constant, Hz: Hertz, DMSO-d6: deuterated dimethylsulfoxide. δ values are expressed in ppm.

The mass spectrometer (MS) was determined using an Agilent (ESI) mass spectrometer, model: Agilent 6120B.

### Preparation Example 1: Preparation of (R)—N—((1S,9S)-4-chloro-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxybutanamide (1-36)

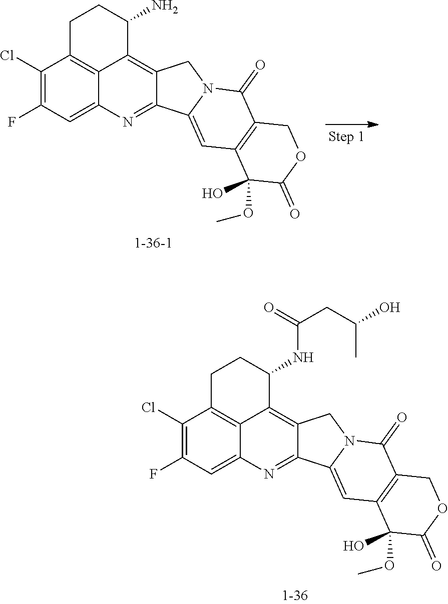 [SMILES: CO[C@@]1(O)C(=O)OCc2c1cc1n(c2=O)Cc2c-1nc1cc(F)c(Cl)c3c1c2[C@@H](N)CC3 ; CO[C@@]1(O)C(=O)OCc2c1cc1n(c2=O)Cc2c-1nc1cc(F)c(Cl)c3c1c2[C@@H](NC(=O)C[C@@H](C)O)CC3]

(1S,9S)-1-Amino-4-chloro-9-ethyl-5-fluoro-9-hydroxy-1,2,3,9,12,15-hexahydro-10H,13H-benzo[d]pyrano[3′,4′:6,7]indolizino[1,2-b]quinolin-10,13-dione (30 mg, 52.64 μmol), (R)-3-hydroxybutyric acid (10.96 mg, 105.28 μmol), HATU (30.02 mg, 78.96 μmol) and DIPEA (20.41 mg, 157.93 μmol) were dissolved in DMF (2 mL), and the reaction system was stirred at 25° C. for 2 hours. The reaction solution was purified by high performance liquid chromatography and freeze-dried to obtain the title compound (17 mg, 31.42 μmol).

Its separation and purification method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 30 |
| 2.00 | 20 | 80 | 30 |
| 18.00 | 85 | 15 | 30 |

Its structural characterization data are as follows:

ESI-MS (m/z): 542.1 [M+H]⁺

¹H NMR (400 MHz, DMSO-d6) δ 8.43 (d, J=8.8 Hz, 1H), 8.06 (d, J=10.4 Hz, 1H), 7.33 (s, 1H), 6.54 (s, 1H), 5.63-5.55 (m, 1H), 5.43 (s, 2H), 5.27 (d, J=18.8 Hz, 1H), 5.21 (d, J=18.8 Hz, 1H), 4.66 (d, J=4.8 Hz, 1H), 4.09-3.98 (m, 1H), 3.32-3.26 (m, 2H), 2.32-2.25 (m, 1H), 2.24-2.12 (m, 3H), 1.93-1.78 (m, 2H), 1.08 (d, J=6.0 Hz, 3H), 0.87 (t, J=7.2 Hz, 3H).

### Preparation Example 2: Preparation of (S)—N-((1S,9S)-4-chloro-9-ethyl-5-fluoro-9-hydroxy-10,13-dioxo-2,3,9,10,13,15-hexahydro-1H,12H-benzo[d]pyrano[3′,4′:6,7]indolizino[1,2-b]quinolin-1-yl)-3-hydroxybutanamide (1-39)

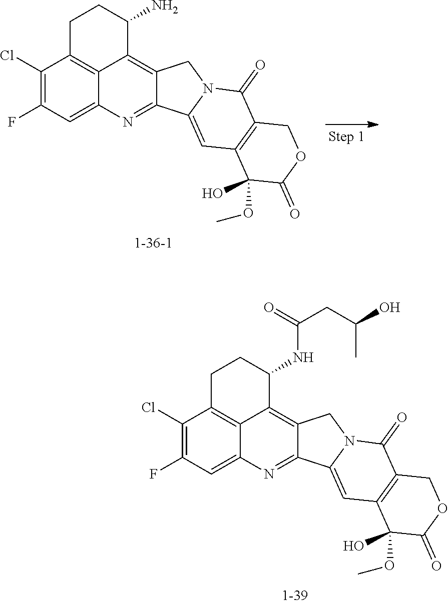 [SMILES: CO[C@@]1(O)C(=O)OCc2c1cc1n(c2=O)Cc2c-1nc1cc(F)c(Cl)c3c1c2[C@@H](N)CC3 ; CO[C@@]1(O)C(=O)OCc2c1cc1n(c2=O)Cc2c-1nc1cc(F)c(Cl)c3c1c2[C@@H](NC(=O)C[C@H](C)O)CC3]

(1S,9S)-1-Amino-4-chloro-9-ethyl-5-fluoro-9-hydroxy-1,2,3,9,12,15-hexahydro-10H,13H-benzo[d]pyrano[3′,4′:6,7]indolizino[1,2-b]quinolin-10,13-dione (30 mg, 0.053 mmol, trifluoroacetate) and (S)-3-hydroxybutyric acid (16.5 mg, 0.158 mmol) were dissolved in DMF (0.5 mL), then added with HATU (40.0 mg, 0.105 mmol) and DIPEA (20.4 mg, 0.158 mmol), and then reacted at room temperature for 2 hours. The reaction solution was directly purified by preparative high performance liquid chromatography and freeze-dried to obtain the title compound (13.5 mg, 0.024 mmol).

Its structural characterization data are as follows:

MS m/z (ESI): 542.1 [M+H]⁺

¹H NMR (400 MHz, DMSO-d6) δ 8.44 (d, J=8.8 Hz, 1H), 8.09 (d, J=10.4 Hz, 1H), 7.34 (s, 1H), 6.56 (m, 1H), 5.64-5.60 (m, 1H), 5.44 (s, 2H), 5.32-5.25 (m, 2H), 4.63 (d, J=4.8H z, 1H), 4.06-4.01 (m, 1H), 3.31-3.26 (m, 2H), 2.28-2.23 (m, 1H), 2.20-2.13 (m, 3H), 1.90-1.82 (m, 2H), 1.08 (d, J=6.4 Hz, 3H), 0.87 (t, J=7.2 Hz, 3H).

Its separation and purification method was as follows:

Chromatographic column: Waters SunFire Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 30 | 70 | 28 |
| 2.00 | 30 | 70 | 28 |
| 18.00 | 90 | 10 | 28 |

### Intermediate Example 1: Preparation of 1-(3,5-bis(2-(methylsulfonyl)pyrimidin-5-yl) phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid (INT-1)

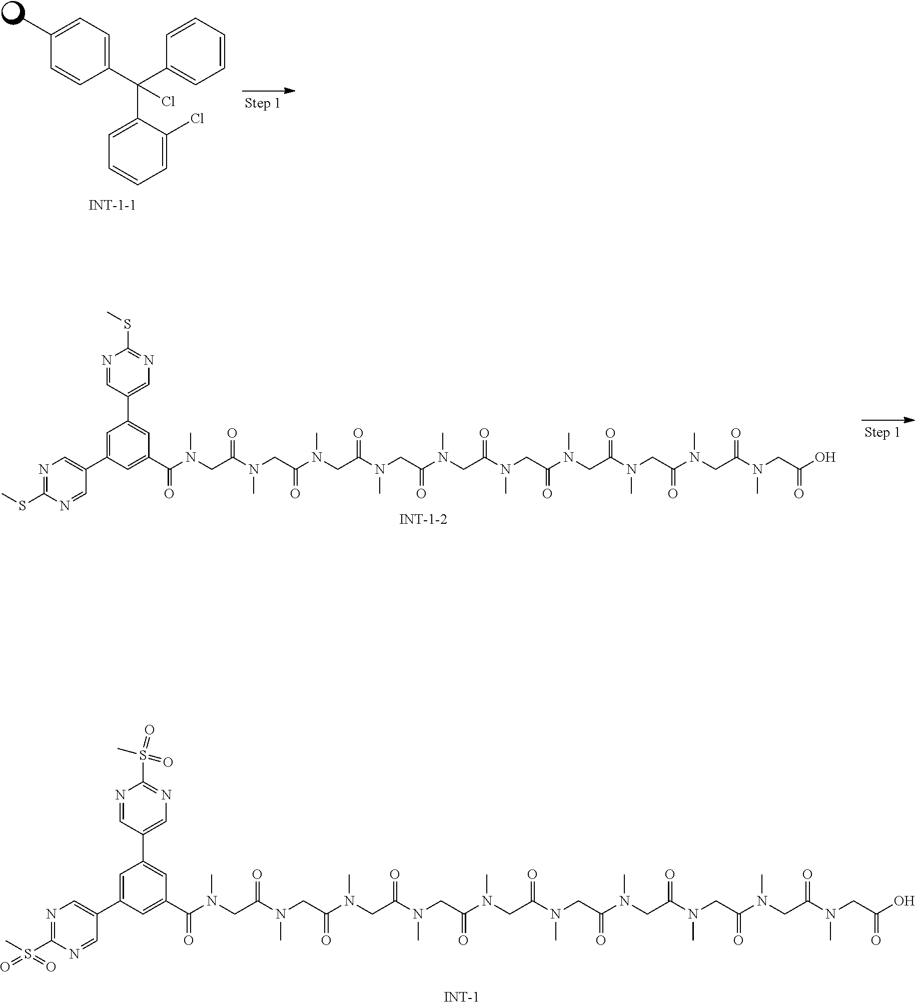 [SMILES: Cc1ccc(C(Cl)(c2ccccc2)c2ccccc2Cl)cc1 ; CSc1ncc(-c2cc(C(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)O)cc(-c3cnc(SC)nc3)c2)cn1 ; CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)c1cc(-c2cnc(S(C)(=O)=O)nc2)cc(-c2cnc(S(C)(=O)=O)nc2)c1 ; "INT-1"]

### Step 1: Preparation of 1-(3,5-bis(2-(methylthio)pyrimidin-5-yl)phenyl)-(2,5,8,11,14,17,20,23,26,29-decamethyl-1,4,7,10,13,16,19,22,25,28-decaoxo-2,5,8,11,14,17,20,23,26,29-decaazahentriacontan-31-oic acid (INT-1-2)

Using standard solid phase synthesis method:

1) Resin preparation: 2-CTC resin (3.00 mmol, 3.70 g, 0.81 mmol/g) and N-(((9H-fluoren-9-yl)methoxy)carbonyl)-N-methylglycine (3.00 mol, 933 mg, 3.00 equivalents) and DIPEA (4.00 equivalents) were added to dichloromethane (10.0 mL), and reacted under nitrogen atmosphere for 2 hours. Then MeOH (1.0 mL) was added to the resin under nitrogen bubbling atmosphere within 30 minutes, and the resin was obtained by filtration.

2) Coupling: under nitrogen bubbling, a DMF (10.0 mL) solution of N-(((9H-fluoren-9-yl)methoxy)carbonyl)-N-methylglycine (5.60 g, 6.00 equivalents) and HATU (6.58 g, 5.70 equivalents) was added to the resin, added dropwise with DIPEA (6.00 equivalents), and then subjected to nitrogen-bubbling at 20° C. for 30 minutes. The resin was washed with DMF (30.0 mL×5) before proceeding to the next step.

3) Deprotection: 20% piperidine DMF solution (30.0 mL) was added to the resin and subjected to nitrogen-bubbling at 20° C. for 30 minutes. The resin was then washed with DMF (30.0 mL×5).

4) Steps 2 and 3 were repeated using the materials and coupling reagents numbered 2 to 10 in Table 1.

5) The resin was then washed with DMF (30.0 mL×5), MeOH (30.0 mL×5), and then dried under vacuum.

TABLE 1

| # | Material | Coupling agent |
|---|---|---|
| 2 | N-(((9H-fluoren-9-yl)methoxy)carbonyl)-N- | HATU (5.70 equiv.) and DIPEA (6.00 |
| | methylglycine (6.00 equiv.) | equiv.) |
| 3 | N-(((9H-fluoren-9-yl)methoxy)carbonyl)-N- | HATU (5.70 equiv.) and DIPEA (6.00 |
| | methylglycine (6.00 equiv.) | equiv.) |
| 4 | N-(((9H-fluoren-9-yl)methoxy)carbonyl)-N- | HATU (5.70 equiv.) and DIPEA (6.00 |
| | methylglycine (6.00 equiv.) | equiv.) |
| 5 | N-(((9H-fluoren-9-yl)methoxy)carbonyl)-N- | HATU (5.70 equiv.) and DIPEA (6.00 |
| | methylglycine (6.00 equiv.) | equiv.) |
| 6 | N-(((9H-fluoren-9-yl)methoxy)carbonyl)-N- | HATU (5.70 equiv.) and DIPEA (6.00 |
| | methylglycine (6.00 equiv.) | equiv.) |
| 7 | N-(((9H-fluoren-9-yl)methoxy)carbonyl)-N- | HATU (5.70 equiv.) and DIPEA (6.00 |
| | methylglycine (6.00 equiv.) | equiv.) |
| 8 | N-(((9H-fluoren-9-yl)methoxy)carbonyl)-N- | HATU (5.70 equiv.) and DIPEA (6.00 |
| | methylglycine (6.00 equiv.) | equiv.) |
| 9 | N-(((9H-fluoren-9-yl)methoxy)carbonyl)-N- | HATU (5.70 equiv.) and DIPEA (6.00 |
| | methylglycine (6.00 equiv.) | equiv.) |
| 10 | 3,5-bis(2-(methylthio)pyrimidin-5- | HATU (1.23 equiv.) and DIPEA (2.60 |
| | yl)benzoic acid (1.30 equiv.) | equiv.) |

Cleavage and purification of peptide:

1) At room temperature, a cleavage solution (TFA/DCM, 1/100, v/v, 200.0 mL) was added to a flask containing the dry resin obtained in 5) of the above solid phase synthesis, stirred for 3 minutes, and filtered, and this step was repeated twice.

2) After filtration, the filtrate was combined and concentrated.

3) The crude product was purified by HPLC, and then freeze-dried to obtain the title compound (1117.1 mg, TFA salt).

Its structural characterization data are as follows:

ESI-MS (m/z): 1010.4 (M+H)⁺

Its purification method was as follows:

Separation condition

| | |
|---|---|
| Dissolution condition | DMF |
| Instrument | Auno DAC-100 |
| Mobile phases | A: water (0.1% TFA in H₂O) |
| | B: acetonitrile |
| Gradient | 20-50%-42 min. Retention time: 21 min |
| Preparative column | Phenomenex Luna C18 250*100 mm, |
| | 10 μm, 100 Å |
| Flow rate | 250 mL/min |
| Detection wavelength | 220/254 nm |
| Temperature | 25° C. |

### Step 2: Preparation of 1-(3,5-bis(2-(methylsulfonyl)pyrimidin-5-yl)phenyl)-2, 5,8,11,14,17,20,23,26,29-decamethyl-1,4,7,10,13,16,19,22,25,28-decaoxo-2,5,8,11,14,17,20,23,26,29-decaazahentriacontan-31-oic acid (INT-1)

1-(3,5-Bis(2-(methylthio)pyrimidin-5-yl)phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid (665 mg, 615.04 μmol) was added to water (7.5 mL) and acetonitrile (15 mL), added with sodium periodate (1.32 g, 6.15 mmol) and ruthenium trichloride hydrate (51.03 mg, 246.02 μmol), and then reacted at 25° C. for 1 hour. The reaction solution was directly purified by reverse phase column (acetonitrile/water (0.05% formic acid)=0-25%) and freeze-dried to obtain the title compound (515 mg, 449.69 μmol).

Its structural characterization data are as follows:

ESI-MS (m/z): 1074.4 (M+H)⁺.

### Intermediate Example 2: Preparation of 1-(3,5-bis(2-(methylsulfonyl)pyrimidin-5-yl) phenyl)-1-oxo-5,8,11,14,17,20,23,26-octaoxa-2-azanonan-29-oic acid (INT-2)

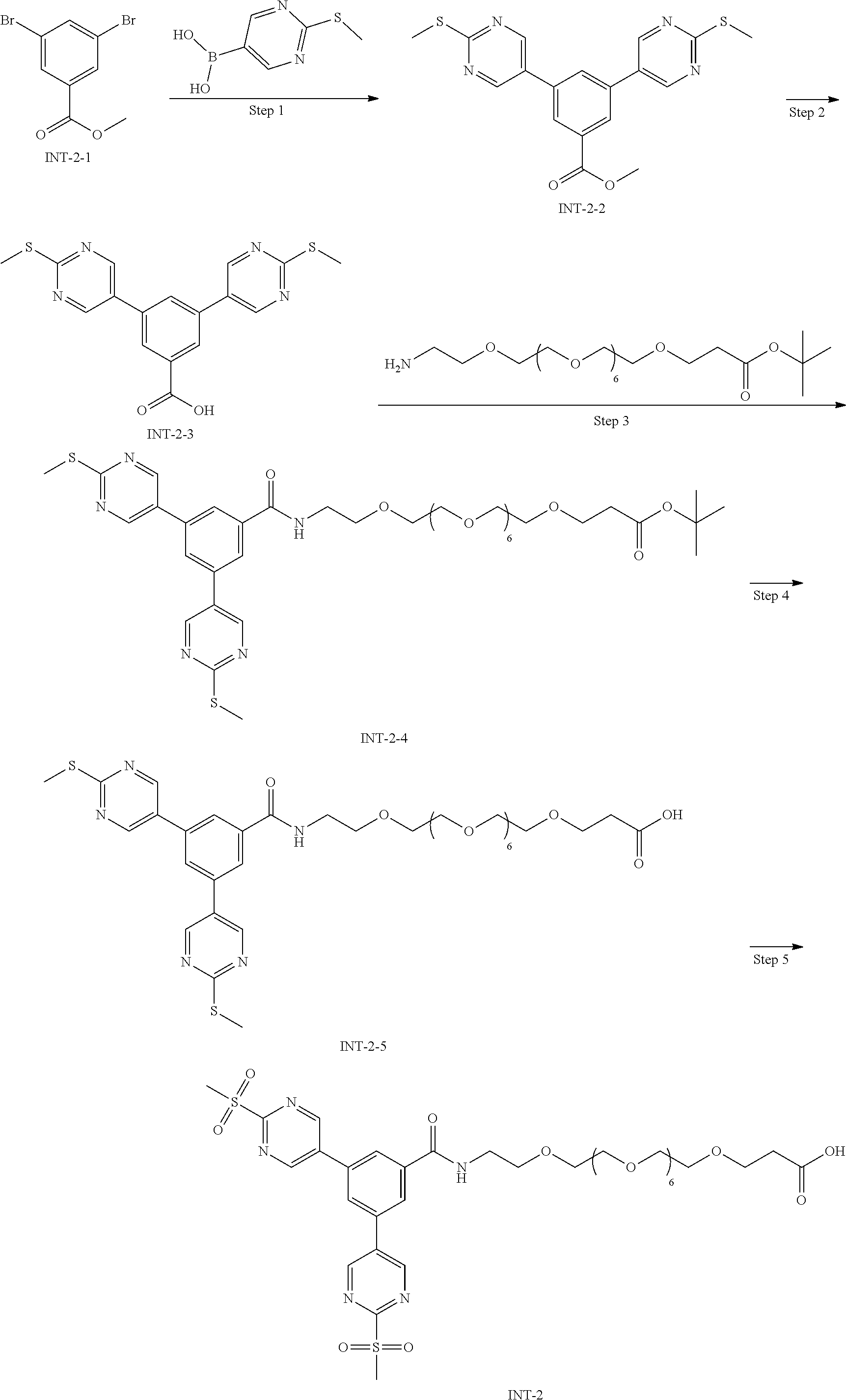

### Step 1: Preparation of methyl 3,5-bis(2-(methylthio)pyrimidin-5-yl)benzoate (INT-2-2)

The raw materials methyl 3,5-dibromobenzoate (720 mg, 2.45 mmol), 2-methylthiopyrimidin-5-boric acid (874 mg, 5.14 mmol), XPhosPd G3 (207 mg, 245 μmol), K₃PO₄ (1.56 g, 7.35 mmol) were added to dioxane (12 mL) and water (4 mL), the reaction system was stirred at 90° C. for 3 hours under nitrogen atmosphere, and LC-MS was used to monitor the reaction. The reaction solution was filtered with diatomaceous earth, the filtrate was added with water and ethyl acetate for extraction, and concentrated to obtain a crude product, which was purified by column chromatography (EA/PE=0-25%) to obtain 710 mg of methyl 3,5-bis(2-(methylthio)pyrimidin-5-yl)benzoate.

Its structural characterization data are as follows:

ESI-MS (m/z): 385.1 [M+H]⁺.

### Step 2: Preparation of 3,5-bis(2-(methylthio)pyrimidin-5-yl)benzoic acid (INT-2-3)

Methyl 3,5-bis(2-(methylthio)pyrimidin-5-yl)benzoate (650 mg, 1.69 mol) and lithium hydroxide (121 mg, 5.07 mmol) were dissolved in THF (2 mL), MeOH (2 mL) and H₂O (2 mL), and reacted under stirring at 25° C. for 2 hours, and the reaction was monitored by LC-MS. The system was adjusted to pH of about 2 with 1N HCl, and a large amount of solid was precipitated and filtered. The filter cake was collected and dried to obtain 560 mg of 3,5-bis(2-(methylthio)pyrimidin-5-yl)benzoic acid.

Its structural characterization data are as follows:

ESI-MS (m/z): 371.1 [M+H]⁺.

### Step 3: Preparation of tert-butyl 1-(3,5-bis(2-(methylthio)pyrimidin-5-yl)phenyl)-1-oxo-5,8,11,14,17,20,23,26-octaoxa-2-azanonacosan-29-oate (INT-2-4)

3,5-Bis(2-(methylthio)pyrimidin-5-yl)benzoic acid (3.00 g, 8.10 mmol) and tert-butyl 1-amino-3,6,9,12,15,18,21,24-octaoxaheptacosan-27-oate (4.03 g, 8.10 mmol) were added to DMF (40 mL), then added with HOBt (3.28 g, 24.3 mmol), EDCI (4.66 g, 24.3 mmol) and DIPEA (4.19 g, 32.4 mmol, 5.64 mL) in sequence, and the reaction system was stirred at 60° C. for 2 hours. The reaction solution was added with water (100 mL) and ethyl acetate for extraction (60 mL×3), and the organic phases were combined and dried over anhydrous sodium sulfate, filtered, and concentrated to obtain tert-butyl 1-(3,5-bis(2-(methylthio)pyrimidin-5-yl) phenyl)-1-oxo-5,8,11,14,17,20,23,26-octaoxa-2-azanonacosan-29-oate (4.20 g, 4.14 mmol), which was used directly in the next step without purification.

### Step 4: Preparation of 1-(3,5-bis(2-(methylthio)pyrimidin-5-yl)phenyl)-1-oxo-5,8,11,14,17,20,23,26-octaoxa-2-azanonacosan-29-oic acid (INT-2-5)

Tert-butyl 1-(3,5-bis(2-(methylthio)pyrimidin-5-yl)phenyl)-1-oxo-5,8,11,14,17,20,23,26-octaoxa-2-azanonacosan-29-oate (3.60 g, 4.24 mmol) was dissolved in dichloromethane (30 mL), added with TFA (15.3 g, 134 mmol, 10 mL), and reacted under stirring at 25° C. for 6 hours. The reaction solution was added with water (60 mL) and ethyl acetate (40 mL×3) for extraction. The organic phases were combined and dried over anhydrous sodium sulfate, filtered, and concentrated to obtain a crude product, which was purified by preparative high performance liquid chromatography and freeze-dried to obtain 1-(3,5-bis(2-(methylthio)pyrimidin-5-yl)phenyl)-1-oxo-5,8,11,14,17, 20,23,26-octaoxa-2-azanonacosan-29-oic acid (2.93 g, 3.63 mmol).

Its structural characterization data are as follows:

ESI-MS (m/z): 794.3 [M+H]⁺.

Its purification method was as follows:

Chromatographic column: Phenomenex luna C18 (250 mm*70 mm*10 μm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 30 | 70 | 30 |
| 15.00 | 60 | 40 | 30 |

### Step 5: Preparation of 1-(3,5-bis(2-(methylsulfonyl)pyrimidin-5-yl)phenyl)-1-oxo-5,8,11,14,17,20,23,26-octaoxa-2-azanonacosan-29-oic acid (INT-2)

1-(3,5-Bis(2-(methylthio)pyrimidin-5-yl)phenyl)-1-oxo-5,8,11,14,17,20,23,26-octaoxa-2-azanonacosan-29-oic acid (148 mg, 0.186 mmol) was added to acetonitrile (15 mL) and water (7.5 mL), then added with sodium periodate (398.71 mg, 1.86 mmol) and ruthenium trichloride hydrate (15.47 mg, 74.56 μmol), and reacted under stirring at 25° C. for 30 minutes. The reaction system was extracted with water and ethyl acetate, and concentrated to obtain 1-(3,5-bis(2-(methylsulfonyl)pyrimidin-5-yl)phenyl)-1-oxo-5,8,11,14,17,20,23,26-octaoxa-2-azanonacosan-29-oic acid (155 mg).

Its structural characterization data are as follows:

ESI-MS (m/z): 858.3 [M+H]⁺.

### Intermediate Example 3: Preparation of allyl (5-amino-2-(((tert-butyldiphenylsilyl)oxy) methyl)benzyl)(methyl)carbamate (INT-3)

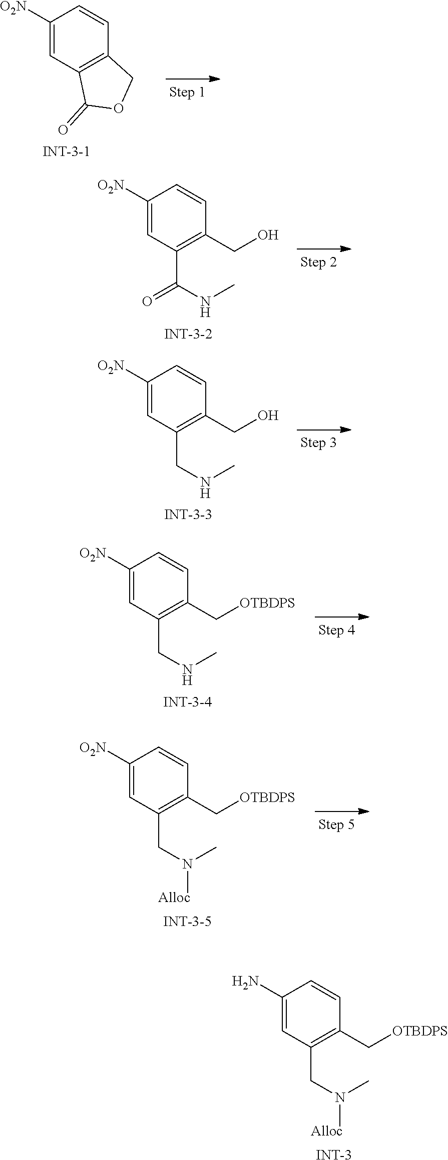

### Step 1: Preparation of 2-hydroxymethyl-N-methyl-5-nitrobenzamide (INT-3-2)

6-Nitroisobenzofuran-1(3H)-one (50.0 g, 279 mmol) was added to a tetrahydrofuran solution of methylamine (2 M, 500 mL), heated to 75° C. and stirred for 5 hours. The reaction solution was directly concentrated to obtain the crude title compound (58.0 g, crude), which was used directly in the next step without purification.

Its structural characterization data are as follows:

ESI-MS (m/z): 211.0 [M+H]⁺.

### Step 2: Preparation of (2-((methylamino)methyl)-4-nitrophenyl)methanol (INT-3-3)

2-Hydroxymethyl-N-methyl-5-nitrobenzamide (25.0 g, 119 mmol) was dissolved in tetrahydrofuran (500 mL). After cooling to 0° C., borane-dimethyl sulfide solution (10 M, 89.3 mL) was added dropwise to the reaction system. After the addition was completed, the temperature was raised to 70° C. and stirred for 5 hours. After the temperature was lowered to 0° C. again, hydrogen chloride-methanol solution (2 M, 100 mL) was added dropwise to the above reaction solution, then heated to 65° C. and stirred for 12 hours. The reaction solution was filtered, and the filtrate was directly concentrated to obtain the crude title compound (44.3 g, crude), which was used directly in the next step without purification.

Its structural characterization data are as follows:

ESI-MS (m/z): 197.0 [M+H]⁺.

### Step 3: Preparation of 1-(2-(((tert-butyldiphenylsilyl)oxy)methyl)-5-nitrophenyl)-N-methylmethylamine (INT-3-4)

(2-((Methylamino)methyl)-4-nitrophenyl)methanol (40.3 g, 205 mmol) was dissolved in dichloromethane (800 mL), cooled to 0° C., added with imidazole (55.9 g, 822 mmol) and tert-butyldiphenylsilyl chloride (84.0 g, 308 mmol, 78.8 mL), then heated to 25° C. and stirred continuously for 1 hour. The reaction system was added with water (200 mL) to quench the reaction, and extracted with dichloromethane 3 times (300 mL×3). The organic phases were combined and washed with saturated sodium chloride aqueous solution (200 mL), dried over anhydrous sodium sulfate, filtered and concentrated to obtain a crude product, which was purified by silica gel column (dichloromethane/methanol=20/1 to 5/1), and concentrated again to obtain the title compound (59.7 g, 137 mmol).

Its structural characterization data are as follows:

ESI-MS (m/z): 435.1 [M+H]⁺.

### Step 4: Preparation of allyl (2-(((tert-butyldiphenylsilyl)oxy)methyl)-5-nitrobenzyl)(methyl)carbamate (INT-3-5)

1-(2-(((Tert-butyldiphenylsilyl)oxy)methyl)-5-nitrophenyl)-N-methylmethylamine (54.9 g, 126 mmol) was dissolved in dichloromethane (550 mL), cooled to 0° C., added with DIPEA (48.9 g, 379 mmol, 66.0 mL) and allyl chloroformate (30.5 g, 253 mmol, 26.8 mL), then heated to 25° C. and continuously stirred for 1 hour. The reaction system was added with water (300 mL) to quench the reaction, and extracted with dichloromethane three times (200 mL×3). The organic phases were combined, washed with saturated sodium chloride aqueous solution (300 mL), dried over anhydrous sodium sulfate, filtered and concentrated to obtain a crude product, which was purified by a silica gel column (petroleum ether/ethyl acetate=20/1~3/1) and concentrated again to obtain the title compound (65.5 g, 126 mmol).

Its structural characterization data are as follows:

ESI-MS (m/z): 519.1 [M+H]⁺.

### Step 5: Preparation of allyl (5-amino-2-(((tert-butyldiphenylsilyl)oxy)methyl) benzyl)(methyl)carbamate (INT-3)

Allyl (2-(((tert-butyldiphenylsilyl)oxy)methyl)-5-nitrobenzyl)(methyl)carbamate (62.8 g, 121 mmol) was dissolved in a mixed solvent of ethanol (300 mL) and water (300 mL), added with iron powder (33.8 g, 605 mmol) and ammonium chloride (64.8 g, 1.21 mol), then heated to 80° C. and stirred for 2 hours. The reaction solution was filtered, and the filtrate was extracted with dichloromethane 3 times (100 mL×3). The organic phases were combined and washed with saturated sodium chloride aqueous solution (300 mL), dried over anhydrous sodium sulfate, then filtered and concentrated to obtain the crude title compound (49.3 g).

Its structural characterization data are as follows:

ESI-MS (m/z): 511.7 [M+H]⁺.

### Intermediate Example 4: (S)-2-amino-N—((S)-1-(((S)-1-)((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)amine)-1-propanamide (A-26-3)

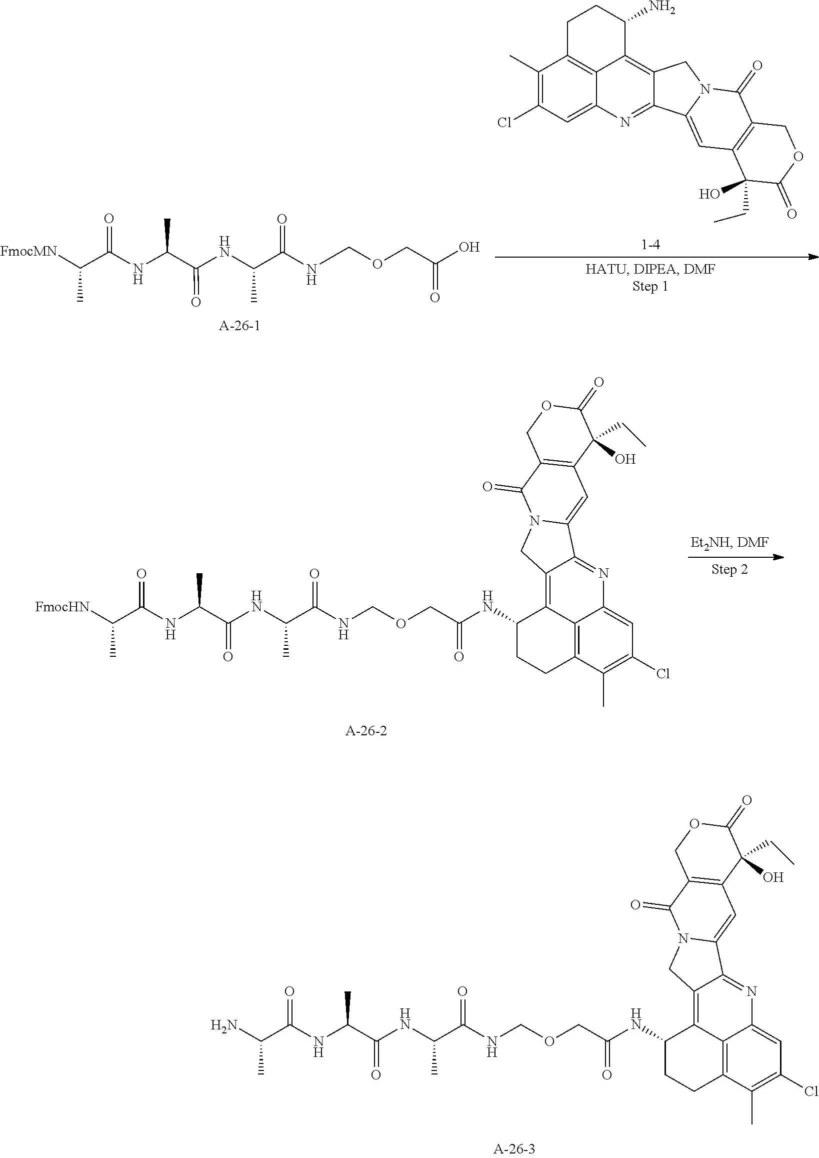

### Step 1

Compound A-26-1 (657 mg, 1.22 mmol) and compound 1-4 (500 mg, 1.11 mmol) were dissolved in N,N-dimethylformamide (10 mL), then added with HATU (630.67 mg, 1.66 mmol) and N,N-diisopropylethylamine (428 mg, 3.32 mmol), and stirred at room temperature for 1 hour.

After the reaction was completed, the reaction solution was directly purified by preparative high performance liquid chromatography and freeze-dried to obtain 700 mg of compound A-26-2.

Its preparation method was as follows:

Chromatographic column: Waters SunFire Prep C18 OBD (5 μm*19 mm*150 mm)

Mobile-phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 30 | 70 | 28 |
| 2.00 | 30 | 70 | 28 |
| 18.00 | 90 | 10 | 28 |

### Step 2

Compound A-26-2 (500 mg, 0.513 mmol) was dissolved in N,N-dimethylformamide (2 mL), then added with diethylamine (75.05 mg, 1.03 mmol), and reacted at room temperature for 1 hour. After the reaction was completed, the reaction solution was directly purified by preparative high performance liquid chromatography and freeze-dried to obtain 307 mg of compound A-26-3.

Its preparation method was as follows:

Chromatographic column: Waters SunFire Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 24 |
| 2.00 | 20 | 80 | 24 |
| 18.00 | 80 | 20 | 24 |

### Example 1: Preparation of N-((7S,10S,13S)-1-(((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-7,1,13-trimethyl-1,6,9,12,15-pentaoxo-3,18,21,24,27,30,33,36,39-nonaoxy-5,8,11,14-tetraazahentetracontan-41-yl)-3,5-bis(2-(methylsulfonyl)pyrimidin-5-yl)benzamide (E-5)

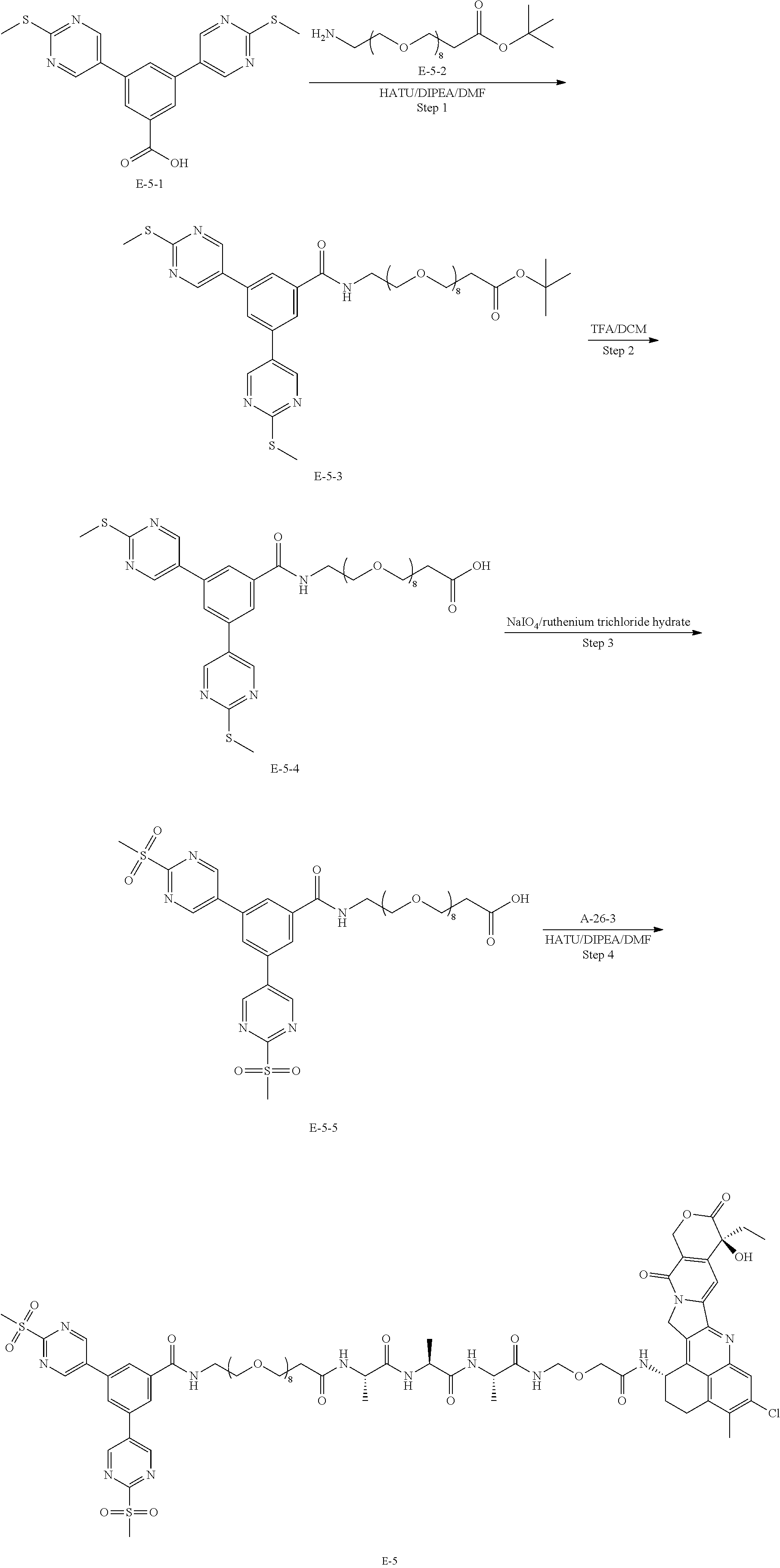

### Step 1

3,5-Bis(2-methylsulfonylpyrimidin-5-yl)benzoic acid (3.00 g, 8.10 mmol) and tert-butyl 1-amino-3,6,9,12,15,18,21,24-octaoxaheptacosan-27-oate (4.03 g, 8.10 mmol) were added to DMF (40 mL), then added with HOBt (3.28 g, 24.3 mmol), EDCI (4.66 g, 24.3 mmol) and DIEA (4.19 g, 32.4 mmol, 5.64 mL) in sequence, and stirred at 60° C. for 2 hours. The reaction solution was added with water (100 mL), and extracted with ethyl acetate (60 mL×3). The organic phases were combined, dried over anhydrous sodium sulfate, filtered and concentrated to obtain compound E-5-3 (4.20 g, 4.14 mmol), which was used directly in the next step without purification.

### Step 2

Tert-butyl 1-(3,5-bis(2-(methylthio)pyrimidin-5-yl)phenyl)-1-oxo-5,8,11,14,17,20,23,26-octaoxa-2-azanonacosan-29-oate (E-5-3, 3.60 g, 4.24 mmol) was dissolved in dichloromethane (30 mL), added with TFA (15.3 g, 134 mmol, 10 mL), and stirred at 25° C. for 6 hours. The reaction solution was added with water (60 mL) and extracted with ethyl acetate (40 mL×3). The organic phases were combined, dried over anhydrous sodium sulfate, filtered and concentrated to obtain a crude product, which was purified by preparative high performance liquid chromatography and freeze-dried to obtain compound E-5-4 (2.93 g, 3.63 mmol).

Its separation and purification method was as follows:

Chromatographic column: Phenomenex luna C18 (250 mm*70 mm*10 μm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 30 | 70 | 30 |
| 15.00 | 60 | 40 | 30 |

### Step 3

1-(3,5-Bis(2-(methylthio)pyrimidin-5-yl)phenyl)-1-oxo-5,8,11,14,17,20,23,26-octaoxa-2-azanonacosan-29-oic acid (E-5-4, 148 mg, 186.41 μmol) was added to acetonitrile (15 mL) and water (7.5 mL), then added with sodium periodate (398.71 mg, 1.86 mmol) and ruthenium trichloride hydrate (15.47 mg, 74.56 μmol), and reacted under stirring at 25° C. for 30 minutes. The reaction was monitored by LC-MS. The reaction solution was added with water and EA for extraction, and concentrated to obtain 155 mg of pale yellow solid compound E-5-5.

### Step 4

(S)-2-Amino-N—((S)-1-(((S)-1-)((2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethoxy)methyl)amino)-1-oxopropan-2-yl)amino)-1-propanamide (A-26-3, 27.91 mg, 34.97 μmol), E-5-5 (30 mg, 34.97 μmol), HATU (26.59 mg, 69.93 μmol) and DIPEA (22.60 mg, 174.84 μmol) were added to DMF (3 mL), and the reaction system was reacted at 25° C. for 1 hour. The reaction solution was purified by preparative high performance liquid chromatography and freeze-dried to obtain 15 mg of the title compound.

Its structural characterization data are as follows:

ESI-MS (m/z): 1591.7 [M+H]⁺

Its separation and purification method was as follows:

Chromatographic column: Waters XBridge Prep C18OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 30 |
| 2.00 | 20 | 80 | 30 |
| 18.00 | 90 | 10 | 30 |

### Example 2: 2,2′,2″-(10-(2-((5-((22S,25S)-1-(3,5-bis(2-(methylsulfonyl)pyrimidin-5-yl) phenyl)-22-isopropyl-25-methyl-1,20,23-trioxo-5,8,11,14,17-pentaoxa-2,21,24-triazahexacosan-26-amido)-2-((((1S,9S)-9-ethyl-5-fluoro-9-hydroxy-4-methyl-10,13-dioxo-2,3,9,10,13,15-hexahydro-1H,12H-benzo[de]pyranol[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-1)

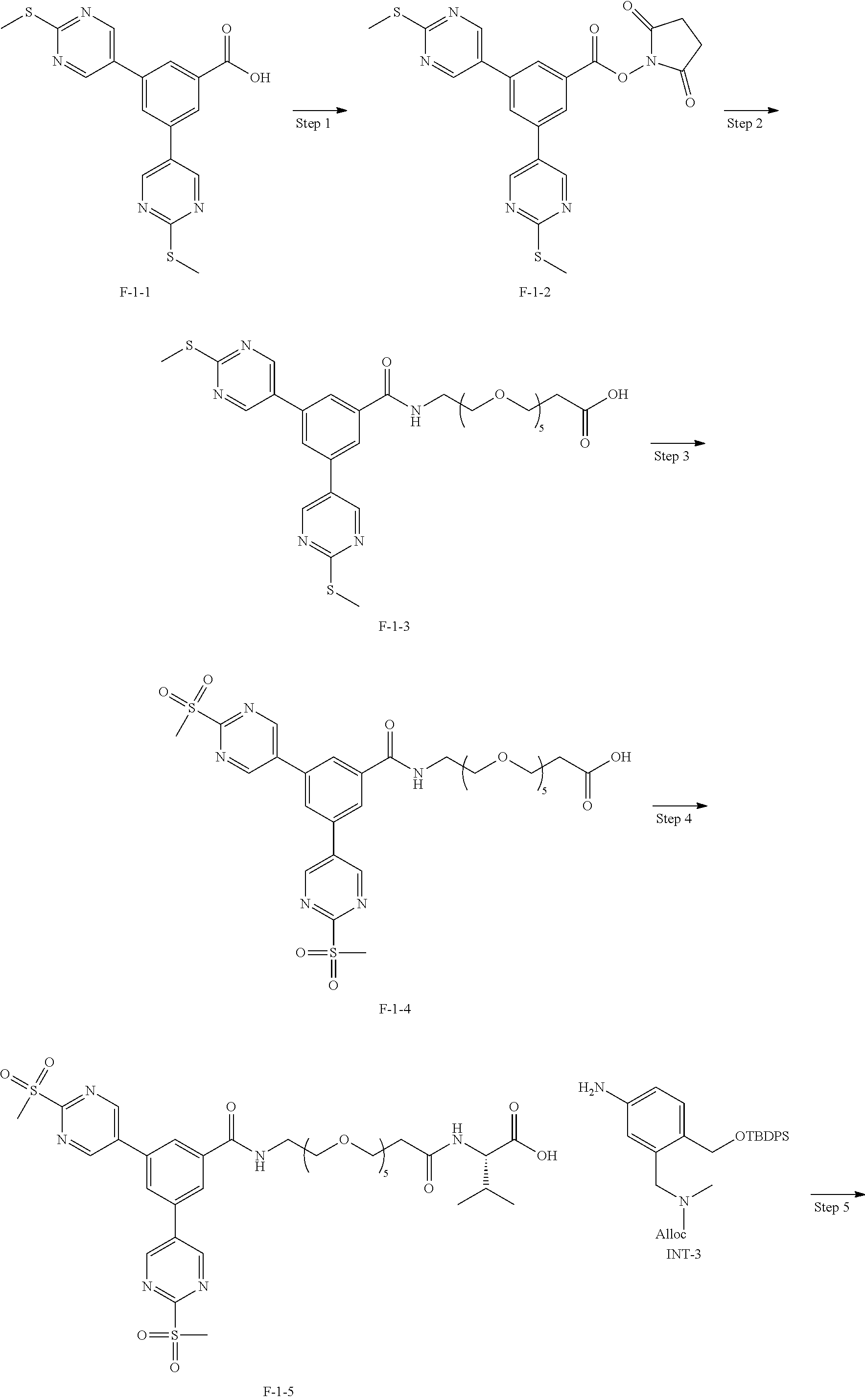

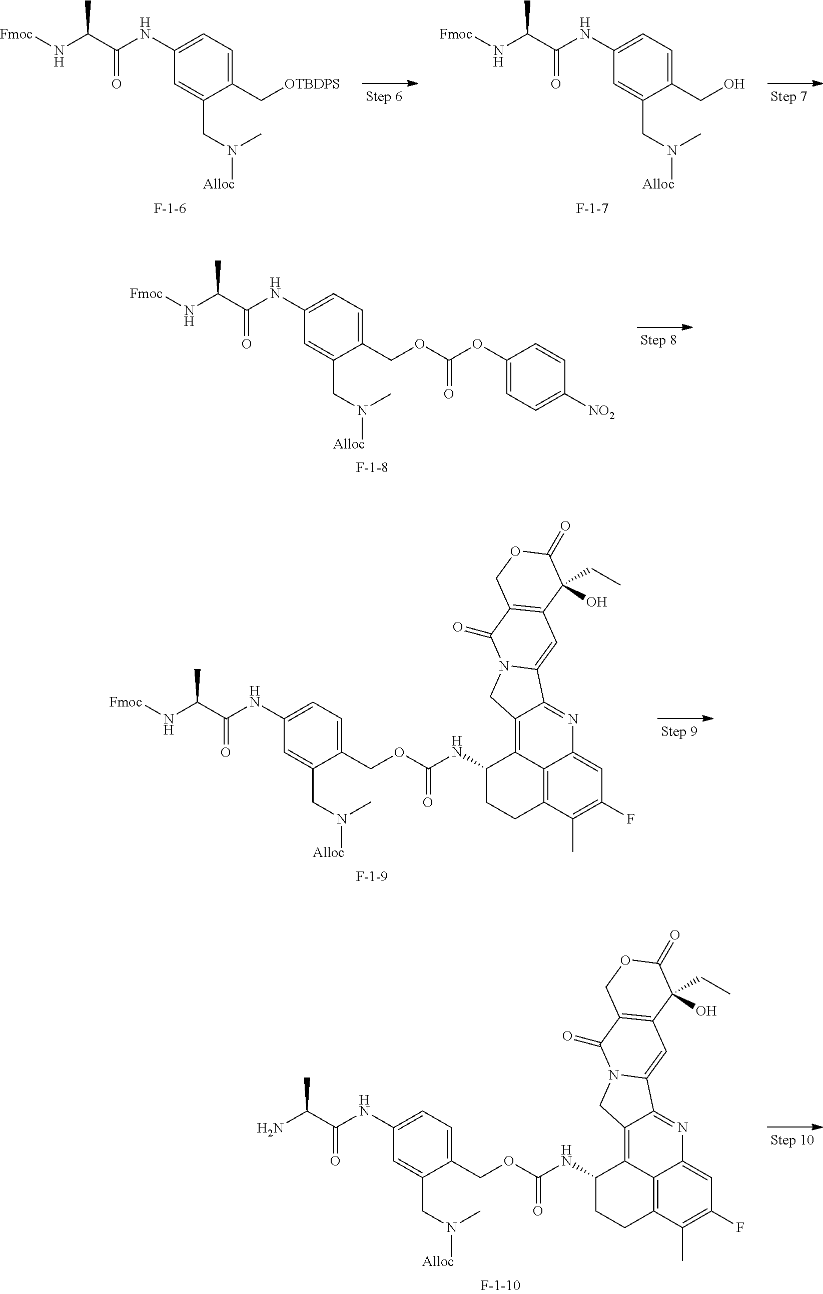

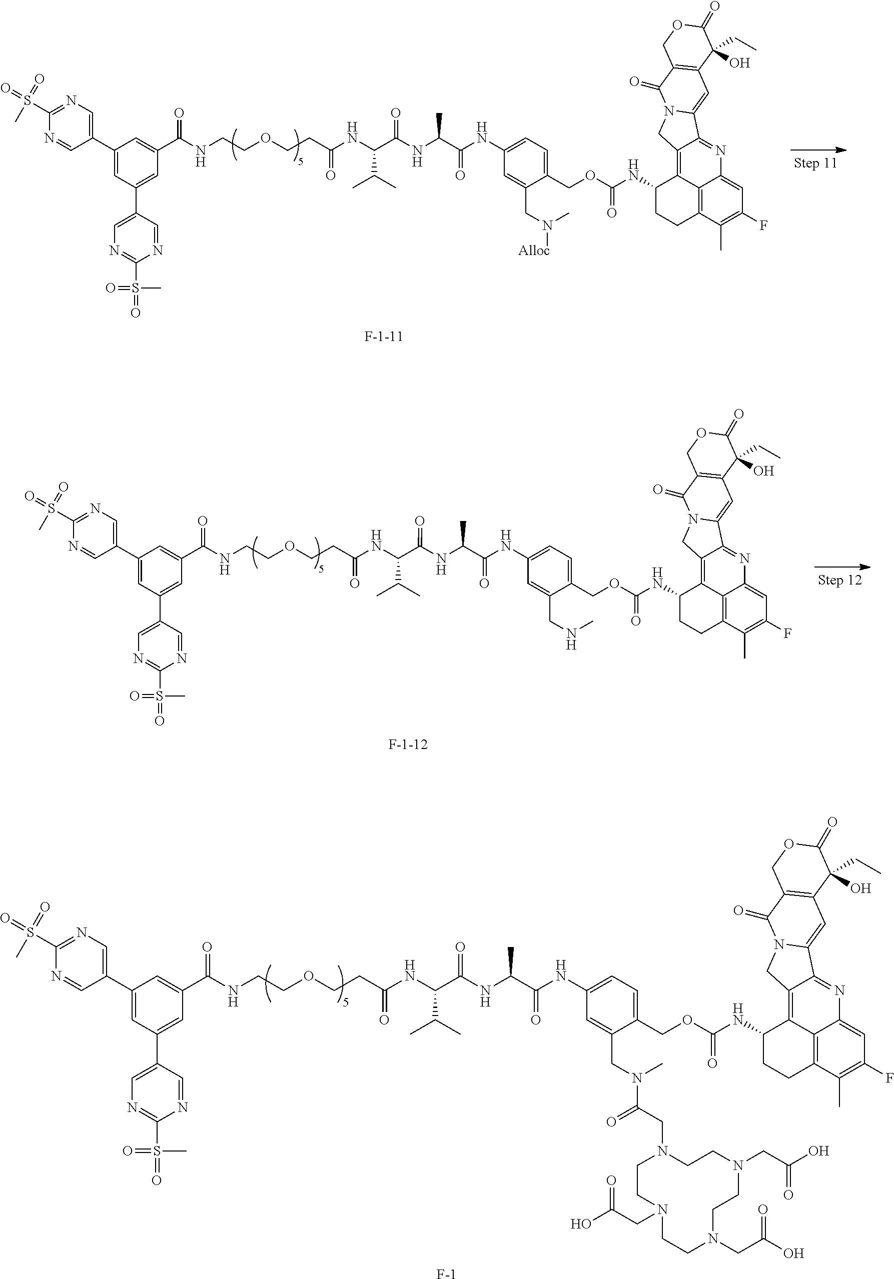

### Step 1: Preparation of 2,5-dioxopyrrolidin-1-yl 3,5-bis(2-(methylthio)pyrimidin-5-yl)benzoate (F-1-2)

3,5-Bis(2-(methylthio)pyrimidin-5-yl)benzoic acid (0.6 g, 1.62 mmol) was dissolved in THF (15 mL), added with N-hydroxysuccinimide (278.89 mg, 2.43 mmol), then added with dicyclohexylcarbodiimide (0.4 g, 1.94 mmol), and stirred at room temperature for 2 hours. After the reaction was completed, the reaction solution was suction filtered, the filtrate was collected and concentrated under reduced pressure to obtain a crude product of the title compound (1.5 g, 3.42 mmol), which was directly used in the next reaction without purification.

### Step 2: Preparation of 1-(3,5-bis(2-(methylthio)pyrimidin-5-yl)phenyl)-1-oxo-5,8,11,14,17-pentaoxa-2-azaeicosan-20-oic acid (F-1-3)

2,5-Dioxopyrrolidin-1-yl 3,5-bis(2-(methylthio)pyrimidin-5-yl)benzoate (331.5 mg, 0.99 mmol) was dissolved in DMF (4 mL), added with 1-amino-3,6,9,12,15-pentaoxaoctadecan-18-oic acid (0.3 g, 0.67 mmol) and DIPEA (588.24 mg, 4.56 mol), and stirred at room temperature for 2 hours. After the reaction was completed, the product was purified by flash column chromatography (C18, water/acetonitrile=0.5) to obtain the title compound (315.50 mg, 0.44 mmol).

Its structural characterization data are as follows:

MS m/z (ESI): 662.2 [M+H]⁺

### Step 3: Preparation of 1-(3,5-bis(2-(methylsulfonyl)pyrimidin-5-yl)phenyl)-1-oxo-5,8,11,14,17-pentaoxa-2-azaeicosan-20-oic acid (F-1-4)

1-(3,5-Bis(2-(methylthio)pyrimidin-5-yl)phenyl)-1-oxo-5,8,11,14,17-pentaoxa-2-azaeicosan-20-oic acid (315.50 mg, 0.44 mmol) was dissolved in acetonitrile (3 mL) and water (1.5 mL), added with sodium periodate (470.50 mg, 2.20 mmol) and ruthenium trichloride monohydrate (9.11 mg, 0.04 mmol), and stirred at room temperature for 0.5 hours. After the reaction was completed, the product was purified by flash column chromatography (C18, water/acetonitrile=2/1) and freeze-dried to obtain the title compound (115.50 mg, 0.16 mmol).

Its structural characterization data are as follows:

MS m/z (ESI): 726.1 [M+H]⁺

### Step 4: Preparation of (1-(3,5-bis(2-(methylsulfonyl)pyrimidin-5-yl)phenyl)-1-oxo-5,8,11,14,17-pentaoxa-2-azaeicosan-20-oyl)-L-valine (F-1-5)

1-(3,5-Bis(2-(methylsulfonyl)pyrimidin-5-yl)phenyl)-1-oxo-5,8,11,14,17-pentaoxa-2-azaeicosan-20-oic acid (115.50 mg, 0.16 mmol) was dissolved in DMF (4 mL), added with 2-(7-azobenzotriazole)-N,N,N′,N′-tetramethyluronium hexafluorophosphate (122.50 mg, 0.32 mmol) and DIPEA (62.0 mg, 0.48 mmol), stirred at room temperature for 0.5 hours, then added with L-valine (50.0 mg, 0.48 mmol), and stirred at room temperature for 15 hours. After the reaction was completed, the product was purified by flash column chromatography (C18, water/acetonitrile=2/1) and freeze-dried to obtain the title compound (30.50 mg, 0.04 mmol).

Its structural characterization data are as follows:

MS m/z (ESI): 825.2 [M+H]⁺

### Step 5: Preparation of allyl (S)-(5-(2-((((9H-fluoren-9-yl)methoxy)carbonyl)amino) propanamido)-2-(((tert-butyldiphenylsilyl)oxy)methyl)benzyl)(methyl)carbamate (F-1-6)

Allyl (5-amino-2-(((tert-butyldiphenylsilyl)oxy)methyl)benzyl)(methyl)carbamate (150.0 mg, 0.31 mmol), (((9H-fluoren-9-yl)methoxy)carbonyl)-L-alanine (115.1 mg, 0.37 mmol) were dissolved in DCM (8 mL) and MeOH (2 mL), added with 2-ethoxy-1-ethoxycarbonyl-1,2-dihydroquinoline (229.7 mg, 0.93 mmol), and stirred at room temperature for 15 hours. The reaction solution was concentrated under reduced pressure to obtain a crude product, which was purified by silica gel column chromatography(ethyl acetate/petroleum ether=0~90%) and then concentrated under reduced pressure again to obtain the title compound (194.2 mg, 0.25 mmol).

Its structural characterization data are as follows:

MS m/z (ESI): 782.2 [M+H]⁺

### Step 6: Preparation of (S)-(5-(2-((((9H-fluoren-9-yl)methoxy)carbonyl)amino) propanamido)-2-(hydroxymethyl)benzyl)(methyl)carbamate (F-1-7)

Allyl (S)-(5-(2-((((9H-fluoren-9-yl)methoxy)carbonyl)amino)propanamido)-2-(((tert-butyldiphenylsilyl)oxy)methyl)benzyl)(methyl)carbamate (194.2 mg, 0.25 mmol) was dissolved in DMF (5 mL), added with pyridine hydrofluoride (390.2 mg, 3.93 mmol), and stirred at room temperature for 15 hours. After the reaction was completed, the reaction solution was added with 20 mL of water, extracted with ethyl acetate 3 times (10 mL×3), washed with 10 mL of brine, dried over anhydrous sodium sulfate and concentrated under reduced pressure to obtain the crude product of the title compound, which was purified by flash column chromatography (C18, water/acetonitrile=2/1) and freeze-dried to obtain the title compound (109.3 mg, 0.21 mmol).

Its structural characterization data are as follows:

MS m/z (ESI): 566.1 [M+Na]+

### Step 7: Preparation of allyl (S)-(5-(2-((((9H-fluoren-9-yl)methoxy)carbonyl)amino) propanamido)-2-((((4-nitrophenoxy)carbonyl)oxy)methyl)benzyl)(methyl)carbamate (F-1-8)

(S)-(5-(2-((((9H-Fluoren-9-yl)methoxy)carbonyl)amino)propanamido)-2-(hydroxymethyl)benzyl) (methyl)carbamate (109.3 mg, 0.21 mmol) was dissolved in DMF (5 mL), added with DIPEA (81.3 mg, 0.63 mmol), then added with p-nitrophenyl chloroformate (50.8 mg, 0.25 mmol), and stirred at room temperature for 2 hours. After the reaction was completed, the product was purified by flash column chromatography (C18, water/acetonitrile=2/1) and freeze-dried to obtain the title compound (134.50 mg, 0.19 mmol).

Its structural characterization data are as follows:

MS m/z (ESI): 731.2 [M+Na]⁺

### Step 8: Preparation of allyl (5-((S)-2-((((9H-fluoren-9-yl)methoxy)carbonyl)amino) propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-1-9)

Allyl (S)-(5-(2-((((9H-fluoren-9-yl)methoxy)carbonyl)amino)propanamido)-2-((((4-nitrophenoxy)carbonyl)oxy)methyl)benzyl)(methyl)carbamate (134.50 mg, 0.19 mmol), (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinolin-10,13-dione (165.50 mg, 0.38 mmol) were dissolved in DMF (5 mL), added with 1-hydroxybenzotriazole (30.80 mg, 0.23 mmol), pyridine (16.5 mg, 0.21 mmol) and DIPEA (73.5 mg, 0.57 mmol), and stirred at room temperature for 2 hours. After the reaction was completed, the product was purified by flash column chromatography (C18, water/acetonitrile=0.7) and freeze-dried to obtain the title compound (114.70 mg, 0.12 mmol).

Its structural characterization data are as follows:

MS m/z (ESI): 1005.2 [M+H]⁺

### Step 9: Preparation of allyl (5-((S)-2-aminopropanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-1-10)

Allyl (5-((S)-2-((((9H-fluoren-9-yl)methoxy)carbonyl)amino)propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (114.70 mg, 0.12 mmol) was dissolved in DMF (3 mL), added with diethylamine (87.8 mg, 1.2 mmol), and stirred at room temperature for 1 hour. After the reaction was completed, the product was purified by flash column chromatography (C18, water/acetonitrile=0.7) and freeze-dried to obtain the title compound (62.6 mg, 0.08 mmol).

Its structural characterization data are as follows:

MS m/z (ESI): 783.1 [M+H]⁺

### Step 10: Preparation of allyl (5-((22S,25S)-1-(3,5-bis(2-(methylsulfonyl)pyrimidin-5-yl) phenyl)-22-isopropyl-25-methyl-1,20,23-trioxo-5,8,11,14,17-pentaoxa-2,21,24-triazahexacosan-26-amido)-2-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)methyl)carbamate (F-1-11)

(1-(3,5-Bis(2-(methylsulfonyl)pyrimidin-5-yl)phenyl)-1-oxo-5,8,11,14,17-pentaoxa-2-azaeicosan-20-oyl)-L-valine (30.50 mg, 0.04 mmol), allyl (5-((S)-2-aminopropanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (39.10 mg, 0.05 mmol) were dissolved in DMF (4 mL), added with 2-(7-azobenzotriazolyl)-N,N,N′,N′-tetramethyluronium hexafluorophosphate (22.80 mg, 0.06 mmol) and DIPEA (15.5 mg, 0.12 mmol), and stirred at room temperature for 1 hour. After the reaction was completed, the product was purified by flash column chromatography (C18, water/acetonitrile=0.6) and freeze-dried to obtain the title compound (31.7 mg, 0.02 mmol).

Its structural characterization data are as follows:

MS m/z (ESI): 1589.3 [M+H]⁺

### Step 11: Preparation of 4-((22S,25S)-1-(3,5-bis(2-(methylsulfonyl)pyrimidin-5-yl)phenyl)-22-isopropyl-25-methyl-1,20,23-trioxo-5,8,11,14,17-pentaoxa-2,21,24-triazahexacosan-26-amido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (F-1-12)

Allyl (5-((22S,25S)-1-(3,5-bis(2-(methylsulfonyl)pyrimidin-5-yl)phenyl)-22-isopropyl-25-methyl-1,20,23-trioxo-5,8,11,14,17-pentaoxa-2,21,24-triazahexacosan-26-amido)-2-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)methyl)carbamate (31.7 mg, 0.02 mmol) was dissolved in DMF (2 mL), added with tetra(triphenylphosphine) palladium (5.2 mg, 4.2 μmol), then added with 15 μL of formic acid and 30 μL of N-methylmorpholine, and stirred at room temperature for 1 hour under nitrogen protection. After the reaction was completed, the product was purified by flash column chromatography (C18, water/acetonitrile=0.6) and freeze-dried to obtain the title compound (12.1 mg, 0.008 mmol).

Its structural characterization data are as follows:

MS m/z (ESI): 1505.2 [M+H]⁺

### Step 12: Preparation of 2,2′,2″-(10-(2-((5-((22S,25S)-1-(3,5-bis(2-(methylsulfonyl) pyrimidin-5-yl)phenyl)-22-isopropyl-25-methyl-1,20,23-trioxo-5,8,11,14,17-pentaoxa-2,21,24-triazahexacosan-26-amido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-1)

4-((22S,25S)-1-(3,5-Bis(2-(methylsulfonyl)pyrimidin-5-yl)phenyl)-22-isopropyl-25-methyl-1,20,23-trioxo-5,8,11,14,17-pentaoxa-2,21,24-triazahexacosan-26-amido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (12.1 mg, 0.008 mmol) was dissolved in DMF (1 mL), added with DIPEA (5.2 mg, 0.04 mmol), and then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (20.1 mg, 0.04 mmol). After the reaction was completed, the reaction solution was directly purified by preparative high performance liquid chromatography and freeze-dried to obtain the title compound (1.0 mg, 0.5 μmol).

Its structural characterization data are as follows:

MS m/z (ESI): 1892.2 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 32 |
| 4.00 | 10 | 90 | 32 |
| 24.00 | 90 | 10 | 32 |

### Example 3: Preparation of N-((20S,23S)-24-(((S)-9-ethyl-9-hydroxy-10,13-dioxo-2,3,9, 10,13,15-hexahydro-1H,12H-benzo[de]pyrano[3′,4′:6,7]indolizino[1,2-b]quinolin-4-yl)amino)-20-isopropyl-23-methyl-18,21,24-trioxo-3,6,9,12,15-pentaoxa-19,22-diazatetracosyl)-3,5-bis(2-(methylsulfonyl)pyrimidin-5-yl)benzamide (G-1)

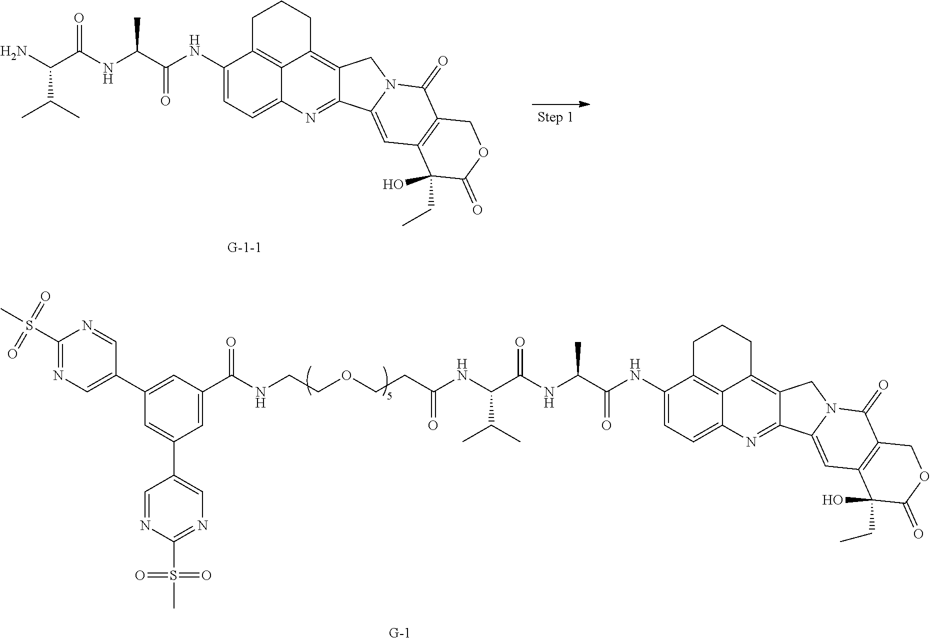

F-1-4 (25.3 mg, 0.03 mmol), (S)-2-amino-N—((S)-1-(((S)-9-ethyl-9-hydroxy-10,13-dioxo-2,3,9,10,13,15-hexahydro)-1H,12H-benzo[de]pyrano[3′,4′:6,7]indolizino[1,2-b]quinolin-4-yl)amino)-1-oxopropan-2-yl)-3-methylbutanamide (20.0 mg, 0.03 mmol) were dissolved in DMF (2 mL), added with 2-(7-azobenzotriazolyl)-N,N,N′,N′-tetramethyluronium hexafluorophosphate (19.9 mg, 0.05 mmol) and DIPEA (13.5 mg, 0.1 mmol), and stirred at room temperature for 1 hour. After the reaction was completed, the reaction solution was directly purified by preparative high performance liquid chromatography and freeze-dried to obtain the title compound (26.9 mg, 0.02 mmol).

Its structural characterization data are as follows:

MS m/z (ESI): 1281.3 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 30 |
| 2.00 | 20 | 80 | 30 |
| 18.00 | 80 | 20 | 30 |

### Example 4: Preparation of N-((29S,32S)-33-(((S)-9-ethyl-9-hydroxy-10,13-dioxo-2,3,9, 10,13,15-hexahydro-1H,12H-benzo[de]pyrano[3′,4′:6,7]indolizino[1,2-b]quinolin-4-yl)amino)-29-isopropyl-32-methyl-27,30,33-trioxo-3,6,9,12,15,18,21,24-octaoxa-28,31-diazatritriacontyl)-3,5-bis(2-(methylsulfonyl)pyrimidin-5-yl)benzamide (G-2)

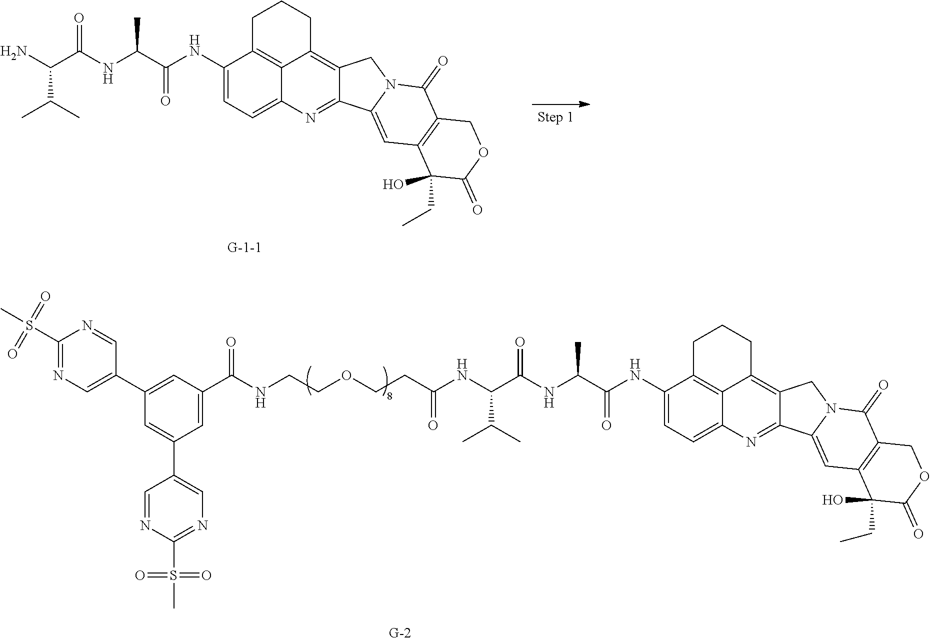

E-5-5 (25.7 mg, 0.03 mmol) and G-1-1 (20.0 mg, 0.03 mmol) were dissolved in DMF (2 mL), added with 2-(7-azobenzotriazole)-N,N,N′,N′-tetramethyluronium hexafluorophosphate (19.9 mg, 0.05 mmol) and DIPEA (13.5 mg, 0.1 mmol), and stirred at room temperature for 1 hour. After the reaction was completed, the reaction solution was directly purified by preparative high performance liquid chromatography and freeze-dried to obtain the title compound (24.5 mg, 0.02 mmol).

Its structural characterization data are as follows:

MS m/z (ESI): 1413.3 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 30 |
| 2.00 | 10 | 90 | 30 |
| 20.00 | 90 | 10 | 30 |

### Example 5: Preparation of N-(2-((2-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizinol[1,2-b]quinolin-4-yl)amino)-1-oxopropan-2-yl)amino)-3-methyl-1-oxobutan-2-yl)amino)-2-oxoethyl)amino)-2-oxoethyl)-3,5-bis(2-(methylsulfonyl)pyrimidin-5-yl)benzamide (G-5)

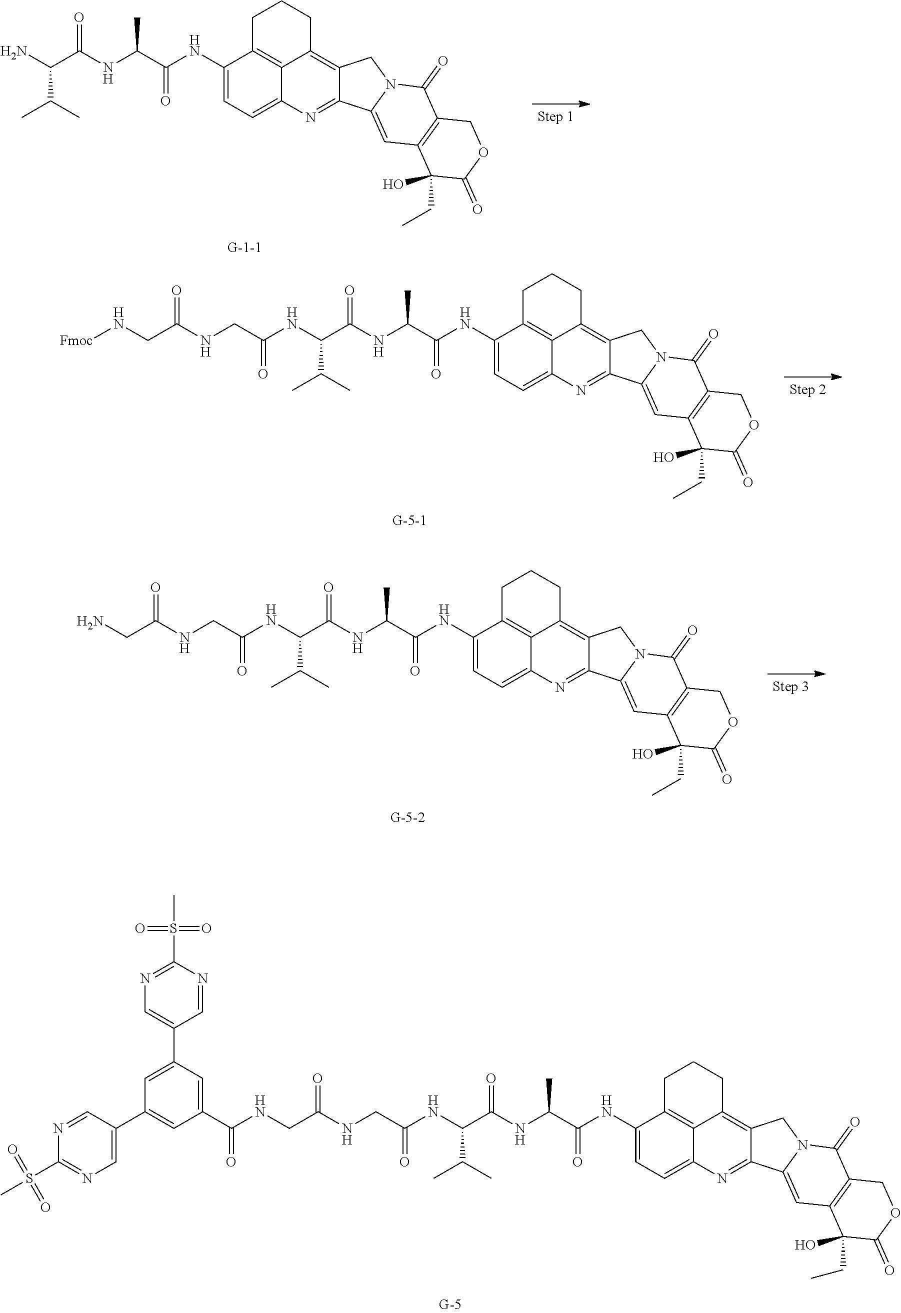

### Step 1: Preparation of (9H-fluoren-9-yl)methyl (2-((2-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-2-oxoethyl)amino)-2-oxoethyl)carbamate (G-5-1)

(((9H-Fluoren-9-yl)methoxy)carbonyl)glycylglycine (10.6 mg, 0.03 mmol) and G-1-1 (20.0 mg, 0.03 mmol) were dissolved in DMF (2 mL), added with 2-(7-azobenzotriazole)-N,N,N′,N′-tetramethyluronium hexafluorophosphate (19.9 mg, 0.05 mmol) and DIPEA (13.5 mg, 0.1 mmol), and stirred at room temperature for 1 hour. After the reaction was completed, 10 mL of water was added, and a solid was precipitated and filtered. The filter cake was collected, freeze-dried to remove water to obtain the title compound (23.5 mg, 0.025 mmol).

Its structural characterization data are as follows:

MS m/z (ESI): 910.3 [M+H]⁺

### Step 2: Preparation of (S)-2-(2-(2-aminoacetamido)acetamido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (G-5-2)

G-5-1 (23.5 mg, 0.025 mmol) was dissolved in DMF (2 mL), added with diethylamine (18.3 mg, 0.25 mmol), and stirred at room temperature for 1 hour. After the reaction was completed, the product was purified by flash column chromatography (C18, water/acetonitrile=0.5) and freeze-dried to obtain the title compound (8.2 mg, 0.012 mmol).

Its structural characterization data are as follows:

MS m/z (ESI): 688.2 [M+H]⁺

### Step 3: Preparation of N-(2-((2-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-2-oxoethyl)amino)-2-oxoethyl)-3,5-bis(2-(methylsulfonyl)pyrimidin-5-yl)benzamide (G-5)

G-5-2 (8.2 mg, 0.012 mmol) was dissolved in DMF (1 mL), added with DIPEA (7.8 mg, 0.06 mmol), and then added with 2,5-dioxopyrrolidin-1-yl 3,5-bis(2-(methylsulfonyl)pyrimidin-5-yl)benzoate (19.1 mg, 0.036 mmol). After the reaction was completed, the reaction solution was directly purified by preparative high performance liquid chromatography and freeze-dried to obtain the title compound (1.45 mg, 1.3 μmol).

Its structural characterization data are as follows:

MS m/z (ESI): 1104.2 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 32 |
| 4.00 | 10 | 90 | 32 |
| 24.00 | 90 | 10 | 32 |

### Example 6: Preparation of N-((31S,34S)-35-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-31-isopropyl-3,6,9,12,15,18,21,24,27,34-decamethyl-2,5,8,11,14,17,20,23,26,29,32,35-dodecaoxo-3,6,9,12,15,18,21,24,27,30,33-undecaazapentatriacontyl)-N-methyl-3,5-bis(2-(methylsulfonyl)pyrimidin-5-yl)benzamide (G-3)

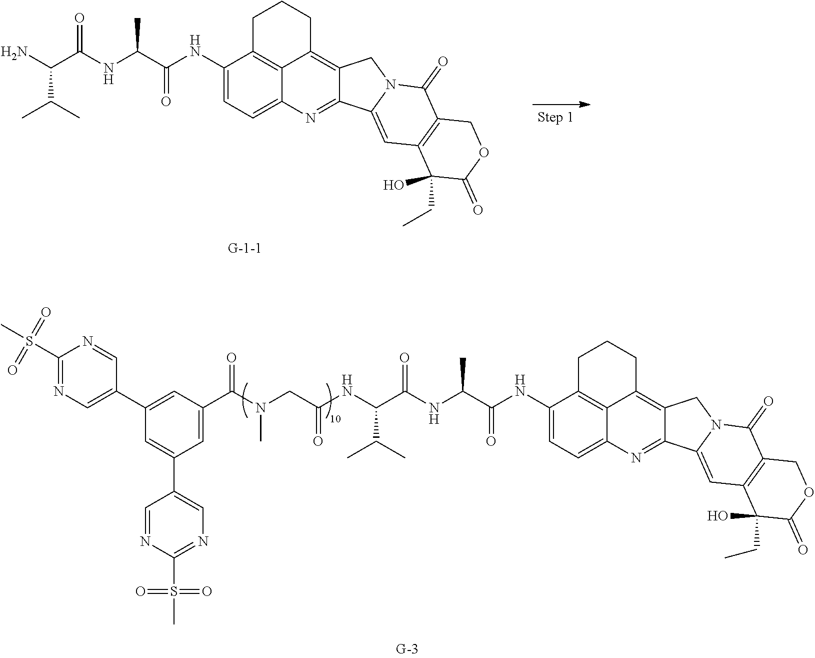

INT-1 (20.0 mg, 17.46 μmol) and G-1-1 (10.0 mg, 17.46 μmol) were dissolved in DMF (1 mL), added with 2-(7-azobenzotriazole)-N,N,N′,N′-tetramethyluronium hexafluorophosphate (8.0 mg, 20.0 μmol) and DIPEA (6.8 mg, 52.39 μmol), and stirred at room temperature for 1 hour. After the reaction was completed, the reaction solution was directly purified by preparative high performance liquid chromatography and freeze-dried to obtain the title compound (3.2 mg, 1.86 μmol).

Its structural characterization data are as follows:

MS m/z (ESI): 1700.6 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 30 |
| 2.00 | 10 | 90 | 30 |
| 20.00 | 90 | 10 | 30 |

### Example 7: Preparation of 2,2′,2″-(10-(2-((5-((22S,25S)-1-(3,5-bis(2-(methylsulfonyl) pyrimidin-5-yl)phenyl)-22-isopropyl-25-methyl-1,20,23-trioxo-5,8,11,14,17-pentaoxa-2,21,24-triazahexacosan-26-amido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-2)

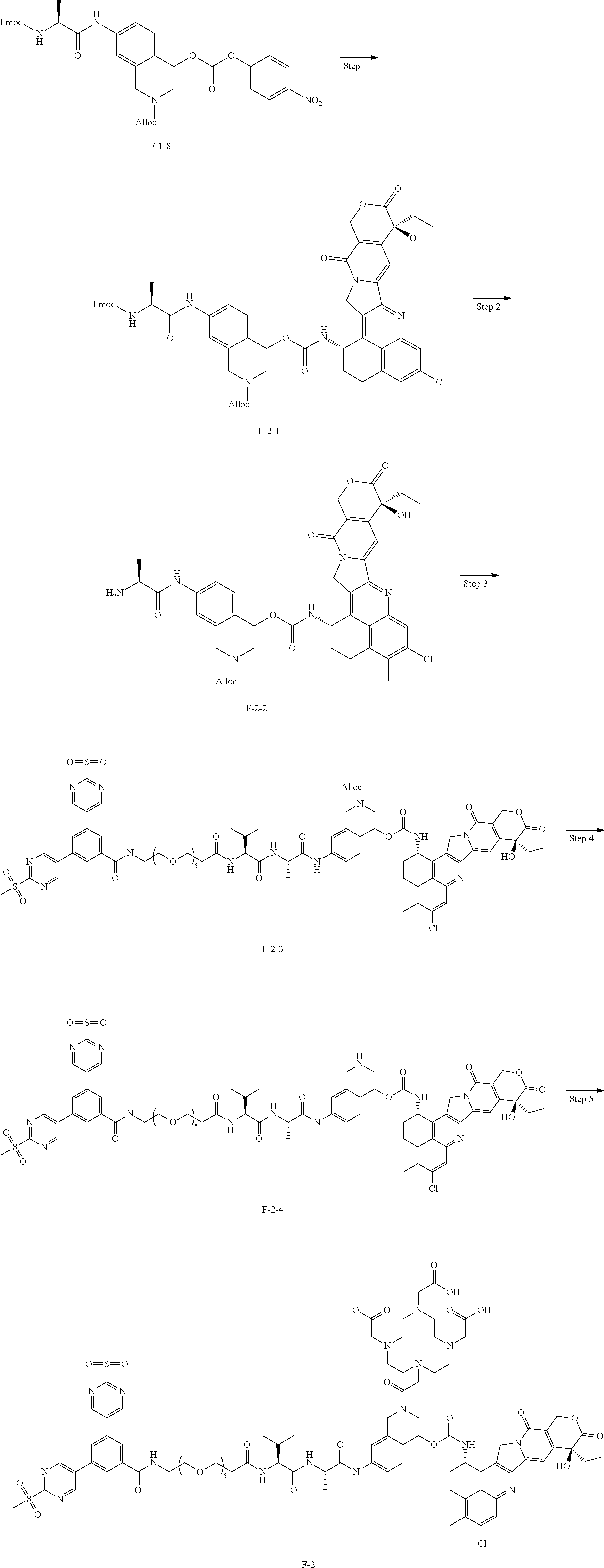

### Step 1: Preparation of allyl (5-((S)-2-((((9H-Fluoren-9-yl)methoxy)carbonyl)amino) propanamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-2-1)

F-1-8 (200.0 mg, 0.28 mmol) and (1S,9S)-1-amino-9-ethyl-5-chloro-9-hydroxy-4-methyl-1,2,3,9,12,15-hexahydro-10H,13H-benzo[de]pyrano[3′,4′:6,7]indolizino[1,2-b]quinolin-10,13-dione (253.0 mg, 0.56 mmol) were dissolved in DMF (6 mL), added with 1-hydroxybenzotriazole (45.5 mg, 0.34 mmol), pyridine (24.5 mg, 0.31 mmol) and DIPEA (108.5 mg, 0.57 mmol), and stirred at room temperature for 2 hours. After the reaction was completed, the product was purified by flash column chromatography (C18, water/acetonitrile=0.7) and freeze-dried to obtain the title compound (183.70 mg, 0.18 mmol).

Its structural characterization data are as follows:

MS m/z (ESI): 1022.2 [M+H]⁺

### Step 2: Preparation of allyl (5-((S)-2-aminopropanamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-2-2)

F-2-1 (183.70 mg, 0.18 mmol) was dissolved in DMF (3 mL), added with diethylamine (131.8 mg, 1.8 mmol), and stirred at room temperature for 1 hour. After the reaction was completed, the product was purified by flash column chromatography (C18, water/acetonitrile=0.6) and freeze-dried to obtain the title compound (136.6 mg, 0.17 mmol).

Its structural characterization data are as follows:

MS m/z (ESI): 800.1 [M+H]⁺

### Step 3: Preparation of allyl (5-((22S,25S)-1-(3,5-bis(2-(methylsulfonyl)pyrimidin-5-yl) phenyl)-22-isopropyl-25-methyl-1,20,23-trioxo-5,8,11,14,17-pentaoxa-2,21,24-triazahexacosan-26-amido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)methyl)carbamate (F-2-3)

F-1-5 (140.13 mg, 0.17 mmol) and F-2-2 (136.6 mg, 0.17 mmol) were dissolved in DMF (5 mL), added with 2-(7-azobenzotriazole)-N,N,N′,N′-tetramethyluronium hexafluorophosphate (97.40 mg, 0.26 mmol) and DIPEA (531.9 mg, 0.51 mmol), and stirred at room temperature for 1 hour. After the reaction was completed, the product was purified by flash column chromatography (C18, water/acetonitrile=0.6) and freeze-dried to obtain the title compound (81.7 mg, 0.05 mmol).

Its structural characterization data are as follows:

MS m/z (ESI): 1606.3 [M+H]⁺

### Step 4: Preparation of 4-((22S,25S)-1-(3,5-bis(2-(methylsulfonyl)pyrimidin-5-yl)phenyl)-22-isopropyl-25-methyl-1,20,23-trioxo-5,8,11,14,17-pentaoxa-2,21,24-triazahexacosan-26-amido)-2-((methylamino)methyl)benzyl ((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (F-2-4)

F-2-3 (81.7 mg, 0.05 mmol) was dissolved in DMF (3 mL), added with tetra(triphenylphosphine) palladium (11.6 mg, 10.0 μmol), then added with 30 μL of formic acid and 60 μL of N-methylmorpholine, and stirred at room temperature for 1 hour under nitrogen protection. After the reaction was completed, the product was purified by flash column chromatography (C18, water/acetonitrile=0.5) and freeze-dried to obtain the title compound (32.1 mg, 0.02 mmol).

Its structural characterization data are as follows:

MS m/z (ESI): 1522.3 [M+H]

### Step 5: Preparation of 2,2′,2″-(10-(2-((5-((22S,25S)-1-(3,5-bis(2-(methylsulfonyl) pyrimidin-5-yl)phenyl)-22-isopropyl-25-methyl-1,20,23-trioxo-5,8,11,14,17-pentaoxa-2,21,24-triazahexacosan-26-amido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-2)

F-2-4 (32.1 mg, 0.02 mmol) was dissolved in DMF (2 mL), added with DIPEA (12.9 mg, 0.1 mmol), and then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (30.1 mg, 0.06 mmol). After the reaction was completed, the reaction solution was directly purified by preparative high performance liquid chromatography and freeze-dried to obtain the title compound (5.91 mg, 3.1 μmol).

Its structural characterization data are as follows:

MS m/z (ESI): 1908.1 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 32 |
| 4.00 | 20 | 80 | 32 |
| 24.00 | 90 | 10 | 32 |

### Example 8: Preparation of 2,2′,2″-(10-(2-((5-((S)-2-((S)-2-(3,5-bis(2-(methylsulfonyl) pyrimidin-5-yl))benzoyl)-3-methylbutanamido)propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-13)

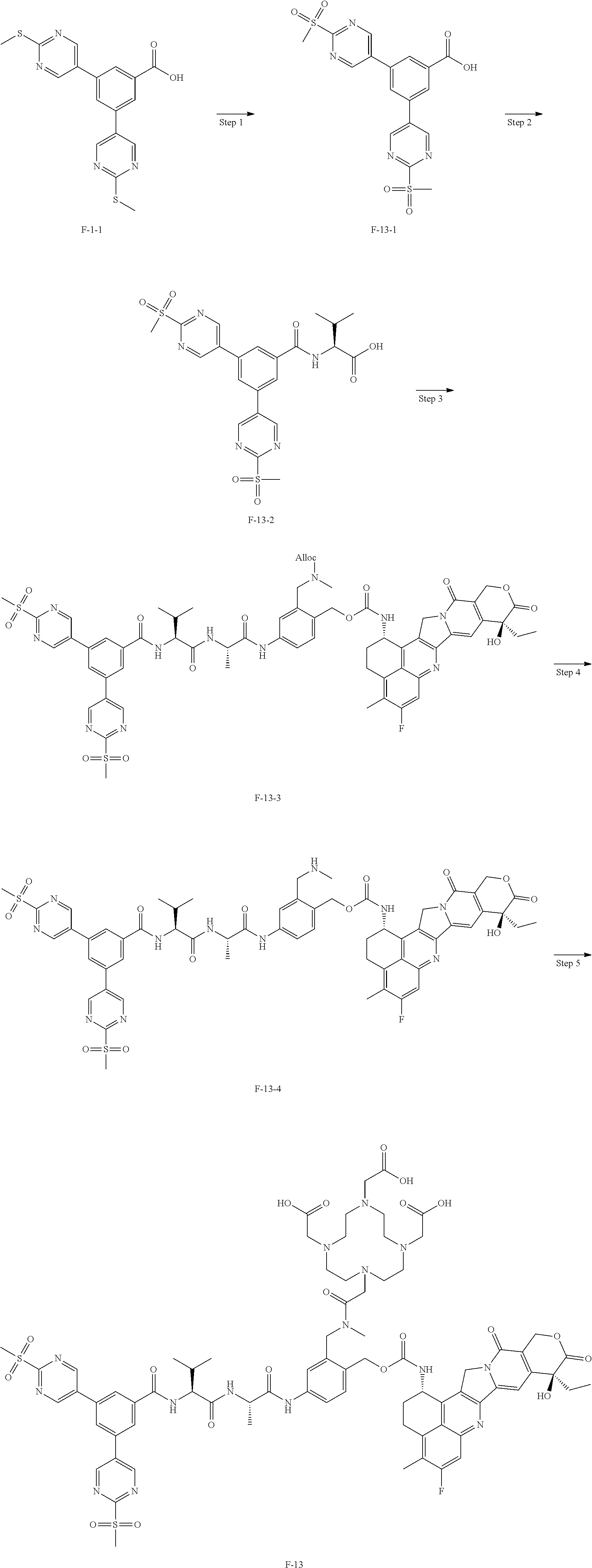

### Step 1: Preparation of 3,5-bis(2-(methylsulfonyl)pyrimidin-5-yl)benzoic acid (F-13-1)

3,5-Bis(2-(methylthio)pyrimidin-5-yl)benzoic acid (0.5 g, 1.35 mmol) was dissolved in a mixed solvent of ACN (20 mL) and water (10 mL), added with sodium periodate (470.50 mg, 2.20 mol) and ruthenium trichloride monohydrate (9.11 mg, 0.04 mmol), and stirred at room temperature for 0.5 hours. After the reaction was completed, the product was purified by flash column chromatography (C18, water/acetonitrile=5/1) and freeze-dried to obtain the title compound (350.50 mg, 0.81 mmol).

Its structural characterization data are as follows:

MS m/z (ESI): 435.0[M+H]⁺

### Step 2: Preparation of (3,5-bis(2-(methylsulfonyl)pyrimidin-5-yl)benzoyl)-L-valine (F-13-2)

F-13-1 (350.50 mg, 0.81 mmol) was dissolved in DMF (3 mL), added with 2-(7-azobenzotriazole)-N,N,N′,N′-tetramethyluronium hexafluorophosphate (342 mg, 0.9 mmol) and DIPEA (313.5 mg, 2.43 mmol), reacted for 10 minutes, then added with L-valine (189.5 mg, 1.62 mmol), and stirred at room temperature for 1 hour. After the reaction was completed, the product was purified by flash column chromatography (C18, water/acetonitrile=0.5) and freeze-dried to obtain the title compound (120.2 mg, 0.22 mmol).

Its structural characterization data are as follows:

MS m/z (ESI): 534.1 [M+H]⁺

### Step 3: Preparation of allyl (5-((S)-2-((S)-2-(3,5-bis(2-(methylsulfonyl)pyrimidin-5-yl) benzamido)-3-methylbutanamido)propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-13-3)

F-13-2 (50.0 mg, 0.094 mmol) and F-1-10 (86.0 mg, 0.11 mmol) were dissolved in DMF (2 mL), added with 2-(7-azobenzotriazole)-N,N,N′,N′-tetramethyluronium hexafluorophosphate (41.8 mg, 0.11 mmol) and DIPEA (36.8 mg, 0.28 mmol), and stirred at room temperature for 1 hour. After the reaction was completed, the product was purified by flash column chromatography (C18, water/acetonitrile=2:1) and freeze-dried to obtain the title compound (62.3 mg, 0.047 mmol).

Its structural characterization data are as follows:

MS m/z (ESI): 1298.4 [M+H]⁺

### Step 4: Preparation of 4-((S)-2-((S)-2-(3,5-bis(2-(methylsulfonyl)pyrimidin-5-yl) benzamido)-3-methylbutanamido)propanamido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (F-13-4)

F-13-3 (62.3 mg, 0.047 mmol) was dissolved in DMF (3 mL), added with tetra(triphenylphosphine) palladium (11.6 mg, 10.0 μmol), then added with 30 μL of formic acid and 60 μL of N-methylmorpholine, and stirred at room temperature for 1 hour under nitrogen protection. After the reaction was completed, the product was purified by flash column chromatography (C18, water/acetonitrile=0.5) and freeze-dried to obtain the title compound (40.6 mg, 0.033 mmol).

Its structural characterization data are as follows:

MS m/z (ESI): 1214.4 [M+H]⁺

### Step 5: Preparation of 2,2′,2″-(10-(2-((5-((S)-2-((S)-2-(3,5-bis(2-(methylsulfonyl) pyrimidin-5-yl))benzamido)-3-methylbutanamido)propanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-13)

F-13-4 (40.6 mg, 0.033 mmol) was dissolved in DMF (2 mL), added with DIPEA (12.9 mg, 0.1 mmol), then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (30.1 mg, 0.06 mmol). After the reaction was completed, the reaction solution was directly purified by preparative high performance liquid chromatography and freeze-dried to obtain the title compound (1.37 mg, 0.85 μmol).

Its structural characterization data are as follows:

MS m/z (ESI): 1600.6 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 32 |
| 4.00 | 20 | 80 | 32 |
| 24.00 | 90 | 10 | 32 |

### Example 9: Preparation of N-((35S,38S)-39-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-35-isopropyl-3,6,9,12,15,18,21,24,27,38-decamethyl-2,5,8,11,14,17,20,23,26,29,33,36,39-tridecaoxy-3,6,9,12,15,18,21,24,27,30,34,37-dodecaazanonatriacontyl)-N-methyl-3,5-bis(2-(methylsulfonyl)pyrimidin-5-yl)benzamide (G-4)

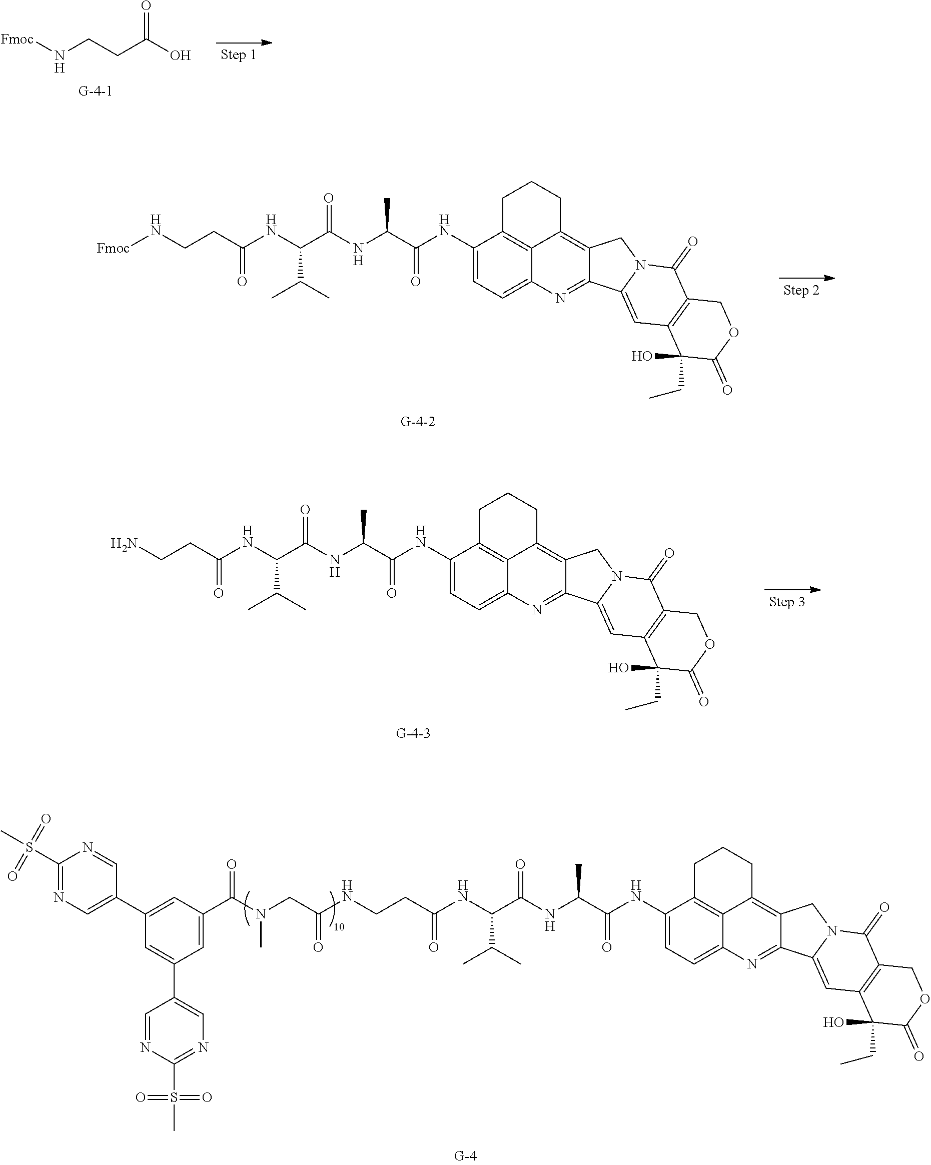

### Step 1: Preparation of (9H-fluoren-9-yl)methyl (3-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutan-2-yl)amino)-3-oxopropyl)carbamate (G-4-2)

3-((((9H-Fluoren-9-yl)methoxy)carbonyl)amino)propionic acid (13.03 mg, 41.84 μmol) and (S)-2-amino-N—((S)-1-(((S)-9-ethyl-9-hydroxy-10,13-dioxo-2,3,9,10,13,15-hexahydro)-1H,12H-benzo[de]pyrano[3′,4′:6,7]indolizino[1,2-b]quinolin-4-yl)amino)-1-oxopropan-2-yl)-3-methylbutanamide (20.0 mg, 34.87 μmol) were dissolved in DMF (2 mL), added with 2-(7-azobenzotriazolyl)-N,N,N′,N′-tetramethyluronium hexafluorophosphate (15.90 mg, 41.84 μmol) and DIPEA (13.52 mg, 104.60 μmol), and stirred at room temperature for 1 hour. After the reaction was completed, the product was purified by flash column chromatography (C18, water/acetonitrile=1:1) and freeze-dried to obtain the title compound (26.0 mg, 29.99 μmol).

Its structural characterization data are as follows:

MS m/z (ESI): 867.4[M+H]⁺

### Step 2: Preparation of (S)-2-(3-aminopropanamido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (G-4-3)

(9H-Fluoren-9-yl)methyl (3-(((S)-1-(((S)-1-(((S)-9-ethyl-9-hydroxy-10,13-dioxo-2,3,9, 10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutan-2-yl)amino)-3-oxopropyl)carbamate (30.0 mg, 34.60 μmol) was dissolved in DMF (1 mL), added with diethylamine (0.5 mL), and stirred at room temperature for 1 hour. After the reaction was completed, the product was purified by flash column chromatography (C18, water/acetonitrile=2:1) and freeze-dried to obtain the title compound (22.31 mg, 34.60 μmol).

Its structural characterization data are as follows:

MS m/z (ESI): 645.6 [M+H]+

### Step 3: Preparation of N-((35S,38S)-39-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-35-isopropyl-3,6,9,12,15,18,21,24,27,38-decamethyl-2,5,8,11,14,17,20,23,26,29,33,36,39-tridecaoxo-3,6,9,12,15,18,21,24,27,30,34,37-dodecaazananotriacontyl)-N-methyl-3,5-bis(2-methylsulfonyl)pyrimidin-5-yl)benzamide (G-4)

(S)-2-(3-aminopropanamido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (22.31 mg, 34.60 μmol) and 1-(3,5-bis(2-(methylsulfonyl) pyrimidin-5-yl)phenyl)-2,5,8,11,14,17,20,23,26,29-decamethyl-1,4,7,10,13,16,19,22,25,28-decaoxo-2,5,8,11,14,17,20,23,26,29-decaazahentriacontan-31-oic acid (22.31 mg, 34.60 μmol) were dissolved in DMF (2 mL), added with 2-(7-azobenzotriazole)-N,N,N′,N′-tetramethyluronium hexafluorophosphate (15.78 mg, 41.53 μmol) and DIPEA (13.42 mg, 103.81 μmol). After the reaction was completed, the reaction solution was directly purified by preparative high performance liquid chromatography and freeze-dried to obtain the title compound (7.90 mg, 4.41 μmol).

Its structural characterization data are as follows:

MS m/z (ESI): 1771.6 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 32 |
| 4.00 | 20 | 80 | 32 |
| 24.00 | 90 | 10 | 32 |

### Example 10: Preparation of 2,2′,2″-(10-(2-((5-((9S,12S)-1-(3,5-bis(2-(methylsulfonyl) pyrimidin-5-yl)phenyl)-9-isopropyl-12-methyl-1,4,7,10-tetraoxo-2,5,8,11-tetraazatridecan-13-amino)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-4)

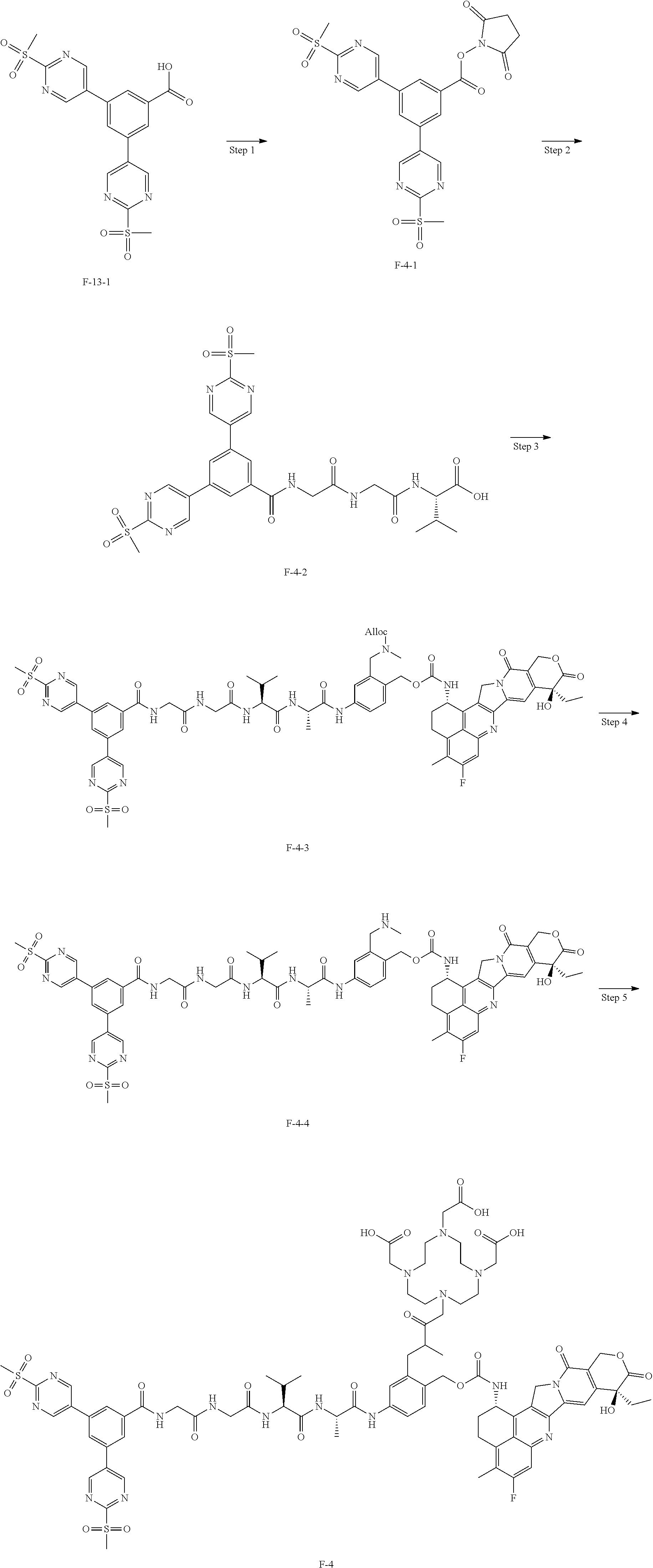

### Step 1: Preparation of 2,5-dioxopyrrolidin-1-yl 3,5-bis(2-(methylsulfonyl)pyrimidin-5-yl)benzoate (F-4-1)

3,5-Bis(2-(methylsulfonyl)pyrimidin-5-yl)benzoic acid (0.1 g, 230.18 μmol) was dissolved in DMF (3 mL), added with EDCI (132.58 mg, 690.54 μmol), and stirred at room temperature for 2 hours. After the reaction was completed, the reaction solution was added with ethyl acetate (20 mL) and water (15 mL), and then the liquids were separated. The organic phase was evaporated under reduced pressure to dryness to obtain the title compound (80 mg, 150.51 μmol).

Its structural characterization data are as follows:

MS m/z (ESI): 532.3[M+H]⁺

### Step 2: Preparation of (S)-2-(((3,5-bis(2-(methylsulfonyl)pyrimidin-5-yl)benzoyl) glycylglycyl)oxy)-3-methylbutanoic acid (F-4-2)

2,5-Dioxopyrrolidin-1-yl 3,5-bis(2-(methylsulfonyl)pyrimidin-5-yl)benzoate (30 mg, 56.44 μmol) was dissolved in DMF (1 mL), added with (2-hydroxyacetyl)glycyl-L-valine (26.22 mg, 112.88 μmol) and DIPEA (14.59 mg, 112.88 μmol), and stirred at room temperature for 1 hour. After the reaction was completed, the product was purified by flash column chromatography (C18, water/acetonitrile=0.5) and then freeze-dried to obtain the title compound (25 mg, 38.54 μmol).

Its structural characterization data are as follows:

MS m/z (ESI): 649.4 [M+H]⁺

### Step 3: Preparation of allyl (5-((9S,12S)-1-(3,5-bis(2-(methylsulfonyl)pyrimidin-5-yl) phenyl)-9-isopropyl-12-methyl-1,4,7,10-tetraoxo-2,5,8,11-tetraazatridecan-13-amino)-2-((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-4-3)

(S)-2-(((3,5-Bis(2-(methylsulfonyl)pyrimidin-5-yl)benzoyl)glycylglycyl)oxy)-3-methylbutanoic acid (25 mg, 38.54 μmol) and (5-((S)-2-aminopropanamido)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (36.20 mg, 46.25 μmol) were dissolved in DMF (2 mL), added with 2-(7-azobenzotriazolyl)-N,N,N′,N′-tetramethyluronium hexafluorophosphate (21.09 mg, 55.50 μmol) and DIPEA (17.93 mg, 138.75 μmol), and stirred at room temperature for 1 hour. After the reaction was completed, the product was purified by flash column chromatography (C18, water/acetonitrile=2:1) and freeze-dried to obtain the title compound (47 mg, 35.26 μmol).

Its structural characterization data are as follows:

MS m/z (ESI): 1413.6 [M+H]⁺

### Step 4: Preparation of 4-((9S,12S)-1-(3,5-bis(2-(methylsulfonyl)pyrimidin-5-yl)phenyl)-9-isopropyl-12-methyl-1,4,7,10-tetraoxo-2,5,8,11-tetraazatridecan-13-amido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (F-4-4)

Allyl (5-((9S,12S)-1-(3,5-bis(2-(methylsulfonyl)pyrimidin-5-yl)phenyl)-9-isopropyl-12-methyl-1,4,7,10-tetraoxo-2,5,8,11-tetraazatridecan-13-amino)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (30 mg, 21.23 μmol) was dissolved in DMF (2 mL), added with tetra(triphenylphosphine) palladium (2.45 mg, 2.12 μmol), then added with 30 μL of formic acid and 60 μL of N-methylmorpholine, and stirred at room temperature for 1 hour under nitrogen protection. After the reaction was completed, the product was purified by flash column chromatography (C18, water/acetonitrile=0.5) and freeze-dried to obtain the title compound (28.22 mg, 21.23 μmol).

Its structural characterization data are as follows:

MS m/z (ESI): 1329.4 [M+H]⁺

### Step 5: Preparation of 2,2′,2″-(10-(2-((5-((9S,12S)-1-(3,5-bis(2-(methylsulfonyl) pyrimidin-5-yl)phenyl)-9-isopropyl-12-methyl-1,4,7,10-tetraoxo-2,5,8,11-tetraazatridecan-13-amino)-2-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-4)

4-((9S,12S)-1-(3,5-Bis(2-(methylsulfonyl)pyrimidin-5-yl)phenyl)-9-isopropyl-12-methyl-1,4,7,10-tetraoxo-2,5,8,11-tetraazatridecan-13-amido)-2-((methylamino)methyl)benzyl ((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (28.22 mg, 21.23 μmol) was dissolved in DMF (2 mL), added with DIPEA (12.9 mg, 0.1 mmol), and then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (83.20 mg, 63.72 μmol). After the reaction was completed, the reaction solution was directly purified by preparative high performance liquid chromatography and freeze-dried to obtain the title compound (5.7 mg, 3.29 μmol).

Its structural characterization data are as follows:

MS m/z (ESI): 1716.8 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 32 |
| 4.00 | 20 | 80 | 32 |
| 24.00 | 90 | 10 | 32 |

### Example 11: Preparation of 2,2′,2″-(10-(2-((5-((S)-2-((S)-2-(3,5-bis(2-(methylsulfonyl) pyrimidin-5-yl)benzamido)-3-methylbutanamido)propanamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-14)

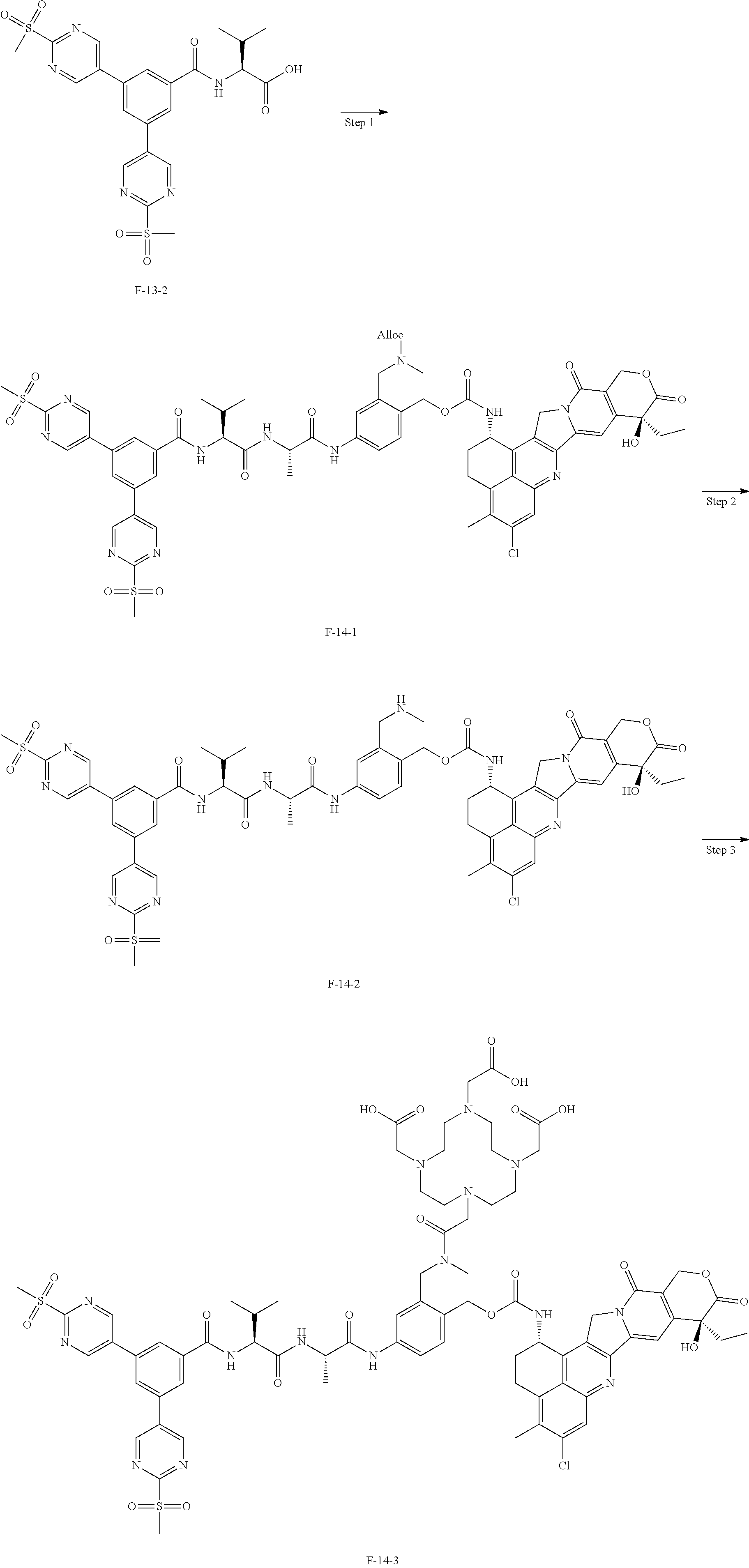

### Step 1: Preparation of allyl (5-((S)-2-((S)-2-(3,5-bis(2-(methylsulfonyl)pyrimidin-5-yl) benzamido)-3-methylbutanamido)propanamido)-2-((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-4-1)

(3,5-Bis(2-(methylsulfonyl)pyrimidin-5-yl)benzoyl)-L-valine (14.80 mg, 27.84 μmol) and allyl (5-((S)-2-aminopropanamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (20.84 mg, 23.20 μmol) were dissolved in DMF (2 mL), added with 2-(7-azobenzotriazolyl)-N,N,N′,N′-tetramethyluronium hexafluorophosphate (12.70 mg, 33.41 μmol) and DIPEA (8.99 mg, 69.59 μmol), and stirred at room temperature for 1 hour. After the reaction was completed, the product was purified by flash column chromatography (C18, water/acetonitrile=2:1) and then freeze-dried to obtain the title compound (26 mg, 19.77 μmol).

Its structural characterization data are as follows:

MS m/z (ESI): 1315.6 [M+H]⁺

### Step 2: Preparation of 4-((S)-2-((S)-2-(3,5-bis(2-(methylsulfonyl)pyrimidin-5-yl) benzamido)-3-methylbutanamido)propanamido)-2-((methylamino)methyl)benzyl ((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (F-14-2)

Allyl (5-((S)-2-((S)-2-(3,5-bis(2-(methylsulfonyl)pyrimidin-5-yl)benzamido)-3-methylbutanamido)propanamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (26 mg, 19.77 μmol) was dissolved in DMF (1 mL), added with tetra(triphenylphosphine) palladium (2.28 mg, 1.98 μmol), then added with 30 μL of formic acid and 60 μL of N-methylmorpholine, and stirred at room temperature for 1 hour under nitrogen protection. After the reaction was completed, the product was purified by flash column chromatography (C18, water/acetonitrile=2:1) and freeze-dried to obtain the title compound (20 mg, 16.25 μmol).

Its structural characterization data are as follows:

MS m/z (ESI): 1231.5 [M+H]+

### Step 3: Preparation of 2,2′,2″-(10-(2-((5-((S)-2-((S)-2-(3,5-bis(2-(methylsulfonyl) pyrimidin-5-yl)benzamido)-3-methylbutanamido)propanamido)-2-(((((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-14)

4-((S)-2-((S)-2-(3,5-Bis(2-(methylsulfonyl)pyrimidin-5-yl)benzamido)-3-methylbutanamido)propanamido)-2-((methylamino)methyl)benzyl ((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (20 mg, 16.25 μmol) was dissolved in DMF (1 mL), added with DIPEA (6.30 mg, 48.75 μmol), and then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (24.45 mg, 48.75 μmol). After the reaction was completed, the reaction solution was directly purified by preparative high performance liquid chromatography and freeze-dried to obtain the title compound (1.14 mg, 0.70 μmol).

Its structural characterization data are as follows:

MS m/z (ESI): 1618.1 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 32 |
| 4.00 | 20 | 80 | 32 |
| 24.00 | 90 | 10 | 32 |

### Example 12: Preparation of 2,2′,2″-(10-(2-((5-((9S,12S)-1-(3,5-bis(2-(methylsulfonyl) pyrimidin-5-yl)phenyl)-9-isopropyl-12-methyl-1,4,7,10-tetraoxo-2,5,8,11-tetraazatridecan-13-amino)-2-((((1S,9S)-9-ethyl-5-chloro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-5)

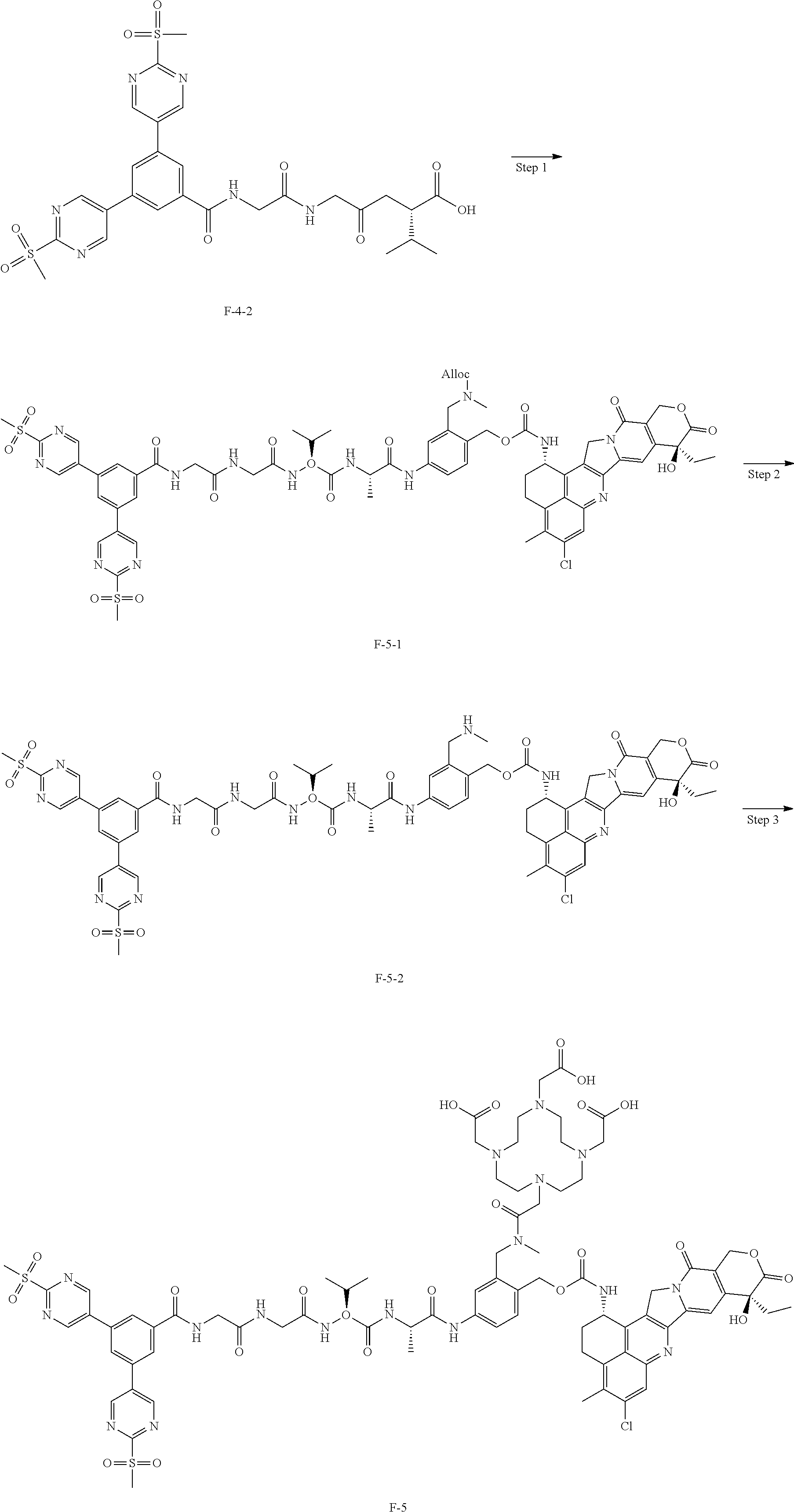

### Step 1: Preparation of allyl (5-((9S,12S)-1-(3,5-bis(2-(methylsulfonyl)pyrimidin-5-yl) phenyl)-9-isopropyl-12-methyl-1,4,7,10-tetraoxo-2,5,8,11-tetraazatridecan-13-amino)-2-((((1S,9S)-9-ethyl-5-chloro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-5-1)

(S)-2-(((3,5-bis(2-(methylsulfonyl)pyrimidin-5-yl)benzoyl)glycylglycyl)oxy)-3-methylbutanoic acid (30 mg, 46.32 μmol) and allyl (5-((S)-2-aminopropanamido)-2-(((((1S,9S)-9-ethyl-5-chloro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (44.43 mg, 55.58 μmol) were dissolved in DMF (2 mL), added with 2-(7-azobenzotriazolyl)-N,N,N′,N′-tetramethyluronium hexafluorophosphate (26.40 mg, 69.48 μmol) and DIPEA (17.96 mg, 138.96 μmol), stirred at room temperature for 1 hour. After the reaction was completed, the product was purified by flash column chromatography (C18, water/acetonitrile=2:1) and freeze-dried to obtain the title compound (30 mg, 20.99 μmol).

Its structural characterization data are as follows:

MS m/z (ESI): 1429.3 [M+H]⁺

### Step 2: Preparation of 4-((9S,12S)-1-(3,5-bis(2-(methylsulfonyl)pyrimidin-5-yl)phenyl)-9-isopropyl-12-methyl-1,4,7,10-tetraoxo-2,5,8,11-tetraazatridecan-13-amido)-2-((methylamino)methyl)benzyl ((1S,9S)-9-ethyl-5-chloro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (F-5-2)

Allyl (5-((9S,12S)-1-(3,5-bis(2-(methylsulfonyl)pyrimidin-5-yl)phenyl)-9-isopropyl-12-methyl-1,4,7,10-tetraoxo-2,5,8,11-tetraazatridecan-13-amino)-2-(((((1S,9S)-9-ethyl-5-chloro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)carbamate (F-5-1) (30 mg, 20.99 μmol) was dissolved in DMF (2 mL), added with tetra(triphenylphosphine) palladium (2.32 mg, 2.01 μmol), then added with 30 μL of formic acid and 60 μL of N-methylmorpholine, and stirred at room temperature for 1 hour under nitrogen protection. After the reaction was completed, the product was purified by flash column chromatography (C18, water/acetonitrile=0.5) and freeze-dried to obtain the title compound (25 mg, 18.59 μmol).

Its structural characterization data are as follows:

MS m/z (ESI): 1331.6 [M+H]⁺

### Step 3: Preparation of 2,2′,2″-(10-(2-((5-((9S,12S)-1-(3,5-bis(2-(methylsulfonyl) pyrimidin-5-yl)phenyl)-9-isopropyl-12-methyl-1,4,7,10-tetraoxo-2,5,8,11-tetraazatridecan-13-amino)-2-((((1S,9S)-9-ethyl-5-chloro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)benzyl)(methyl)amino)-2-oxoethyl)-1,4,7,10-tetraazacyclododecan-1,4,7-triyl)triacetic acid (F-5)

4-((9S,12S)-1-(3,5-Bis(2-(methylsulfonyl)pyrimidin-5-yl)phenyl)-9-isopropyl-12-methyl-1,4,7,10-tetraoxo-2,5,8,11-tetraazatridecan-13-amido)-2-((methylamino)methyl)benzyl ((1S,9S)-9-ethyl-5-chloro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamate (F-5-2) (10 mg, 7.44 μmol) was dissolved in DMF (2 mL), added with DIPEA (2.88 mg, 22.31 μmol), and then added with 2,2′,2″-(10-(2-((2,5-dioxopyrrolidin-1-yl)oxy)-2-oxoethyl)-1,4,7,10-tetraazacyclododecan-1,4,7-triyl)triacetic acid (11.19 mg, 22.31 μmol). After the reaction was completed, the reaction solution was directly purified by preparative high performance liquid chromatography and freeze-dried to obtain the title compound (1.01 mg, 3.29 μmol).

Its structural characterization data are as follows:

MS m/z (ESI): 1732.2 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 32 |
| 4.00 | 20 | 80 | 32 |
| 24.00 | 90 | 10 | 32 |

### Example 13: Preparation of 2,2′,2″-(10-(2-(((S)-5-(3,5-bis(2-(methylsulfonyl)pyrimidin-5-yl) benzamido)-6-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino-6-oxohexyl)amino)-2-oxoethyl)-1,4,7,10-tetraazacyclododecan-1,4,7-triyl)triacetic acid (G-25)

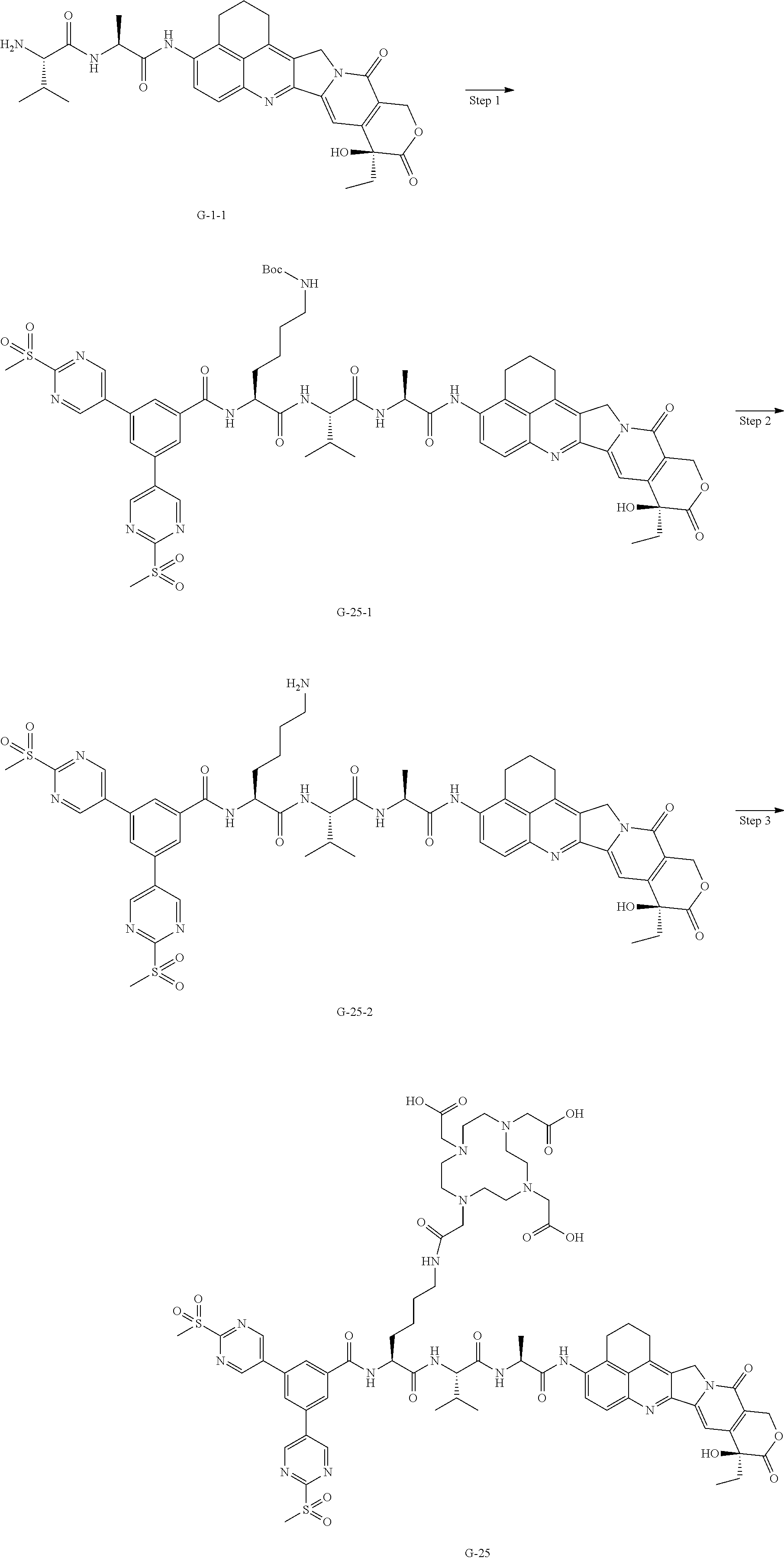

### Step 1: Preparation of tert-butyl ((S)-5-(3,5-bis(2-(methylsulfonyl)pyrimidin-5-yl) benzamido)-6-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-6-oxohexyl)carbamate (G-25-1)

N²-(3,5-Bis(2-(methylsulfonyl)pyrimidin-5-yl)benzoyl)-N⁶-(tert-butoxycarbonyl)-L-lysine (64.12 mg, 96.75 μmol) and (S)-2-amino-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (G-1-1) (37 mg, 64.50 μmol) were dissolved in DMF (2 mL), then added with DIPEA (25.01 mg, 193.50 μmol) and HATU (36.79 mg, 96.75 μmol), and reacted at room temperature for 1 hour. After the reaction was completed, the reaction solution was directly purified by preparative high performance liquid chromatography and freeze-dried to obtain the title compound (20 mg, 16.42 μmol).

Its structural characterization data are as follows:

MS m/z (ESI): 1219.3 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 0 | 100 | 30 |
| 2.00 | 10 | 90 | 30 |
| 16.00 | 90 | 10 | 30 |

### Step 2: Preparation of tert-butyl ((S)-5-(3,5-bis(2-(methylsulfonyl)pyrimidin-5-yl) benzamido)-6-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-6-oxohexyl)carbamate (G-25-2)

Tert-butyl ((S)-5-(3,5-bis(2-(methylsulfonyl)pyrimidin-5-yl)benzamido)-6-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-6-oxohexyl)carbamate (G-25-1) (20 mg, 16.42 μmol) was dissolved in DCM (1 mL), added with TFA (0.1 mL), and reacted at room temperature for 2 hours. After the reaction was completed, the reaction solution was directly purified by preparative high performance liquid chromatography and freeze-dried to obtain the title compound (20 mg, 16.42 μmol).

Its structural characterization data are as follows:

MS m/z (ESI): 1119.3 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 0 | 100 | 30 |
| 4.00 | 10 | 90 | 30 |
| 16.00 | 90 | 10 | 30 |

### Step 3: Preparation of 2,2′,2″-(10-(2-(((S)-5-(3,5-bis(2-(methylsulfonyl)pyrimidin-5-yl) benzamido)-6-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino-6-oxohexyl)amino)-2-oxoethyl)-1,4,7,10-tetraazacyclododecan-1,4,7-triyl)triacetic acid (G-25)

2,2′,2″-(10-(2-(2,5-Dioxopyrrolidin-1-yl)-2-oxoethyl)-1,4,7,10-tetraazacyclododecan-1,4,7-triyl)triacetic acid (2.02 mg, 4.02 μmol) and tert-butyl ((S)-5-(3,5-bis(2-(methylsulfonyl) pyrimidin-5-yl)benzamido)-6-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-6-oxohexyl)carbamate (G-25-2) (3.00 mg, 2.68 μmol) were dissolved in DMF (1 mL), then added with DIPEA (346.72 μg, 2.68 μmol), and reacted at room temperature for 1 hour. After the reaction was completed, the reaction solution was directly purified by preparative high performance liquid chromatography and freeze-dried to obtain the title compound (1.06 mg, 6.55e−1 μmol).

Its structural characterization data are as follows:

MS m/z (ESI): 1504.8 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 0 | 100 | 30 |
| 2.00 | 5 | 95 | 30 |
| 24.00 | 90 | 10 | 30 |

### Example 14: Preparation of 2,2′,2″-(10-((2S,5S,14S)-14-(3,5-bis(2-(methylsulfonyl) pyrimidin-5-yl)benzamido)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,10,13,20-hexaoxo-3,6,9,12,19-pentaazaheneicosan-21-yl)-1,4,7,10-tetraazacyclododecan-1,4,7-triyl)triacetic acid (G-26)

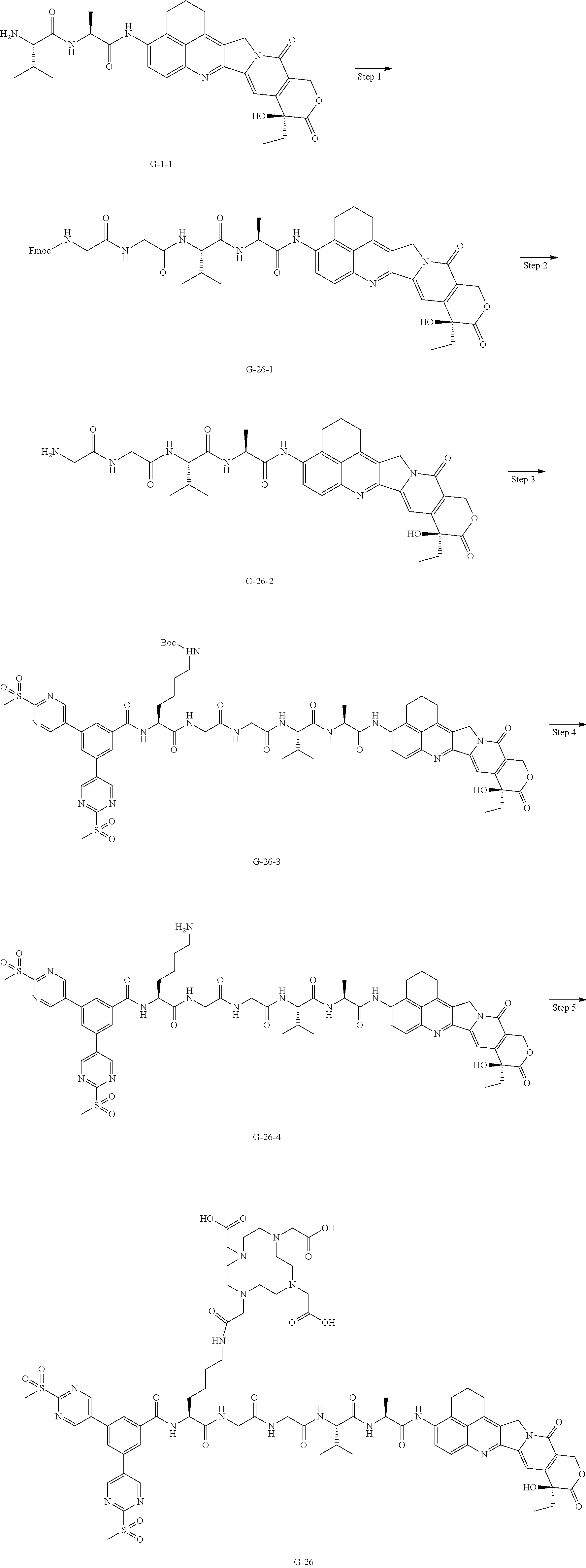

### Step 1: Preparation of (9H-fluoren-9-yl)methyl (2-((2-(((S)-1-(((S)-1-(((S)-9-ethyl-9-hydroxy-10,13)-dioxo-2,3,9,10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutan-2-yl)amino)-2-oxoethyl)amino)-2-oxoethyl)carbamate (G-26-1)

(((9H-Fluoren-9-yl)methoxy)carbonyl)glycylglycine (31.88 mg, 89.95 μmol) and (S)-2-amino-N—((S)-1-(((S)-9-ethyl-9-hydroxy-10,13-dioxo-2,3,9,10,13,15-hexahydro)-1H,12H-benzo[de]pyrano[3′,4′:6,7]indolizino[1,2-b]quinolin-4-yl)amino)-1-oxopropan-2-yl)-3-methylbutanamide (G-1-1) (43 mg, 74.96 μmol) were dissolved in DMF (1 mL), added with HATU (34.20 mg, 89.95 μmol), then added with DIPEA (29.06 mg, 224.88 μmol), and reacted at room temperature for 1 hour. The reaction solution was evaporated under vacuum to remove the solvent to obtain the crude title compound, which was directly used in the next step without purification.

Its structural characterization data are as follows:

MS m/z (ESI): 910.5 [M+H]⁺

### Step 2: Preparation of (S)-2-(2-(2-aminoacetamido)acetamido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (G-26-2)

The crude product of (9H-fluoren-9-yl)methyl (2-((2-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropyl-2-yl)amino)-3-methyl-1-oxobutan-2-yl)amino)-2-oxoethyl)amino)-2-oxoethyl)carbamate (G-26-1) was dissolved in DMF (2.42 mL), added with DEA (54.65 mg, 747.27 μmol, 77.30 μL), and reacted at room temperature for 1 hour. The reaction solution was evaporated under vacuum to remove the solvent to obtain a crude product. The crude product was purified by flash column chromatography (C18, water/acetonitrile=0.7) and freeze-dried to obtain the title compound (38 mg, 55.25 μmol).

Its structural characterization data are as follows:

MS m/z (ESI): 689.2 [M+H]⁺

### Step 3: Preparation of tert-butyl ((2S,5S,14S)-14-(3,5-bis(2-(methylsulfonyl)pyrimidin-5-yl) benzamido)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,10,13-pentaoxo-3,6,9,12-tetraazaoctadecan-18-yl)carbamate (G-26-3)

N²-(3,5-Bis(2-(methylsulfonyl)pyrimidin-5-yl)benzoyl)-N⁶-(tert-butoxycarbonyl)-L-lysine (54.93 mg, 82.88 μmol) and (S)-2-(2-(2-aminoacetamido)acetamido)-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (G-26-2) (38 mg, 55.25 μmol) were dissolved in DMF (2 mL), then added with HATU (31.51 mg, 82.88 μmol) and DIPEA (14.28 mg, 110.51 μmol), and reacted at room temperature for 4 hours. After the reaction was completed, the reaction solution was directly purified by preparative high performance liquid chromatography and freeze-dried to obtain the title compound (27 mg, 20.26 μmol).

Its structural characterization data are as follows:

MS m/z (ESI): 1333.4 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 0 | 100 | 30 |
| 4.00 | 30 | 70 | 30 |
| 24.00 | 90 | 10 | 30 |

### Step 4: Preparation of N-((2S,5S,14S)-18-amino-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,10,13-pentaoxo-3,6,9,12-tetraazaoctadecan-14-yl)-3,5-bis(2-(methylsulfonyl)pyrimidin-5-yl)benzamide (G-26-4)

Tert-butyl ((2S,5S,14S)-14-(3,5-bis(2-(methylsulfonyl)pyrimidin-5-yl)benzamido)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,10,13-pentaoxo-3,6,9,12-tetraazaoctadecan-18-yl)carbamate (G-26-3) (27 mg, 20.26 μmol) was dissolved in DCM (2 mL), added with TFA (0.2 mL), and reacted at room temperature for 2 hours. After that, the reaction solution was evaporated to remove solvent to obtain the crude title compound, which was directly used in to the next step without purification.

Its structural characterization data are as follows:

MS m/z (ESI): 1233.4 [M+H]⁺

### Step 5: Preparation of 2,2′,2″-(10-((2S,5S,14S)-14-(3,5-bis(2-(methylsulfonyl) pyrimidin-5-yl)benzamido)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,10,13,20-hexaoxo-3,6,9,12,19-pentaazaheneicosan-21-yl)-1,4,7,10-tetraazacyclododecan-1,4,7-triyl)triacetic acid (G-26)

2,2′,2″-(10-(2-(2,5-Dioxopyrrolidin-1-yl)-2-oxoethyl)-1,4,7,10-tetraazacyclododecan-1,4,7-triyl)triacetic acid (14.18 mg, 29.21 μmol) and the crude product of N-((2S,5S,14S)-18-amino-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-5-isopropyl-2-methyl-1,4,7,10,13-pentaoxo-3,6,9,12-tetraazaoctadecan-14-yl)-3,5-bis(2-(methylsulfonyl)pyrimidin-5-yl)benzamide (G-26-4) were dissolved in DMF (2 mL), then added with DIPEA (7.55 mg, 58.43 μmol), and reacted at room temperature for 2 hours. After the reaction was completed, the reaction solution was directly purified by preparative high performance liquid chromatography and freeze-dried to obtain the title compound (15.30 mg, 8.79 μmol).

Its structural characterization data are as follows:

MS m/z (ESI): 1619.9 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 0 | 100 | 30 |
| 4.00 | 10 | 90 | 30 |
| 24.00 | 70 | 30 | 30 |

### Example 15: Preparation of pentafluorophenol 1-(N—((S)-1-(((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-2-oxoethoxy)carbonyl)aminosulfonyl)piperidin-4-oate (1-50)

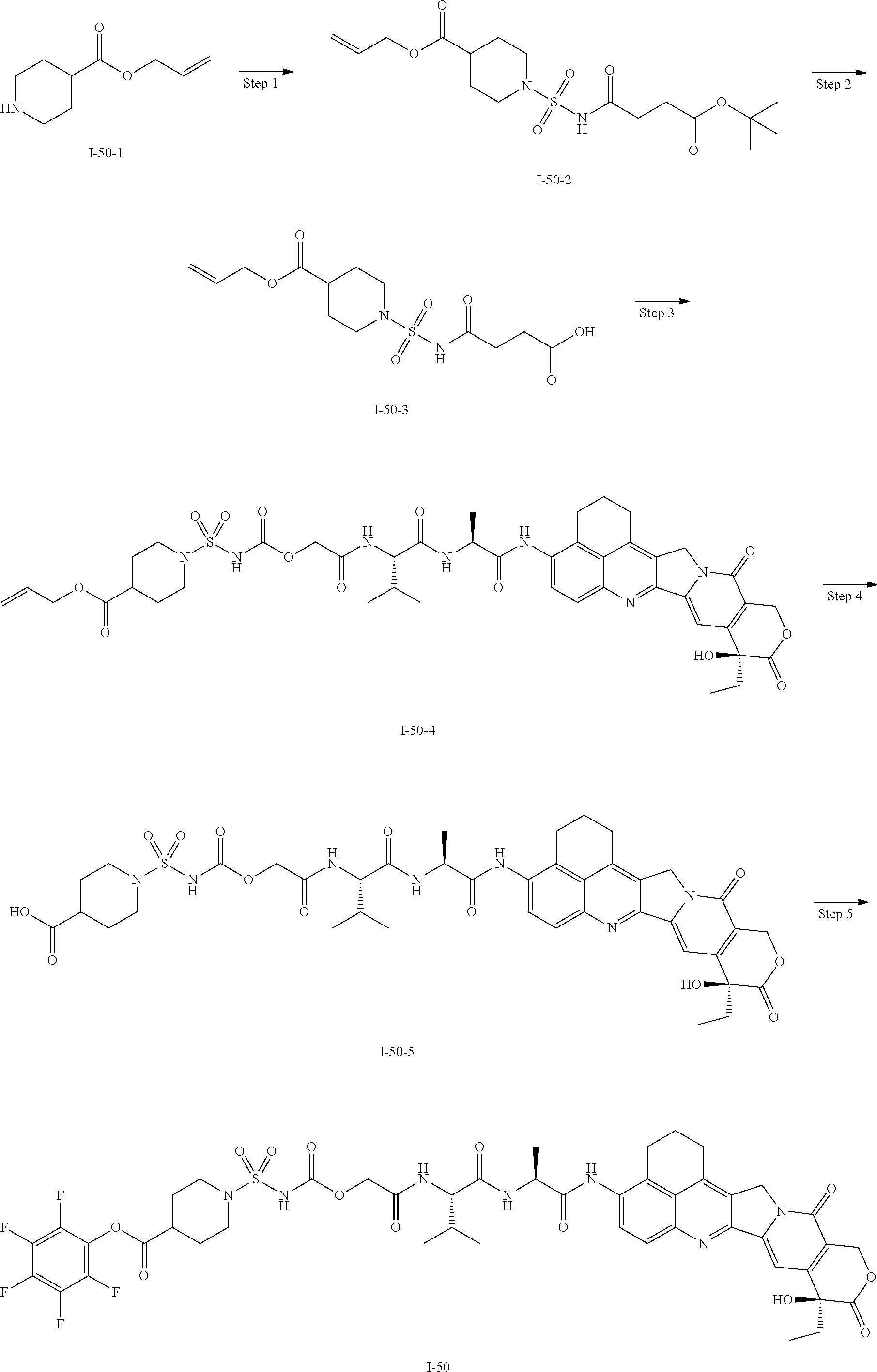

### Step 1

Chlorosulfonyl isocyanate (2.14 g, 15.13 mmol) was added to dichloromethane (50 mL), cooled in an ice-water bath for 10 minutes, then added with tert-butyl 2-hydroxy acetate (2 g, 15.13 mmol) into the above reaction system, and reacted under stirring in an ice-water bath for 2 hours. The reaction system was added with allyl piperidin-4-oate hydrochloride (3.74 g, 18.16 mmol) and triethylamine (4.59 g, 45.40 mmol), the ice-water bath was removed, and the reaction system naturally returned to room temperature and stirred continuously for 3 hours. The reaction system was added with water (100 mL) to quench the reaction, and extracted with ethyl acetate 3 times (50 mL×3). The organic phases were combined and dried with anhydrous sodium sulfate, filtered and concentrated to obtain a crude product, which was purified by silica gel column (DCM/PE=0-100%), and concentrated again to obtain compound I-50-2 (1.87 g, 4.19 mmol).

Its structural characterization data are as follows:

¹H NMR (400 MHz, DMSO) δ 11.64 (s, 1H), 6.06-5.79 (m, 1H), 5.35-5.17 (m, 2H), 4.61-4.48 (m, 4H), 4.03 (q, J=7.1 Hz, 1H), 3.59 (dt, J=12.4, 3.3 Hz, 2H), 3.04-2.88 (m, 2H), 1.93 (dd, J=13.5, 3.2 Hz, 2H), 1.59 (dd, J=13.4, 3.6 Hz, 2H), 1.43 (s, 9H).

### Step 2

Allyl 1-(N-((2-(tert-butoxy)-2-oxoethoxy)carbonyl)aminosulfonyl)piperidin-4-oate (I-50-2) (1.75 g, 4.31 mmol) was added to a mixed solvent of trifluoroacetic acid (5 mL) and dichloromethane (10 mL), and reacted at 25° C. for 2 hours. The reaction system was concentrated to dryness, added with ethyl acetate for dilution, adjusted to pH of about 8 with sodium bicarbonate aqueous solution, extracted to remove some impurities, then adjusted to pH of about 3 with 3N dilute hydrochloric acid, and extracted with ethyl acetate 3 times (30 mL×3). The organic phases were combined and dried over anhydrous sodium sulfate, filtered and concentrated to obtain compound I-50-3 (1.4 g, 4.00 mmol).

Its structural characterization data are as follows:

ESI-MS (m/z): 351.1 [M+H]⁺.

### Step 3

2-((((4-((Allyloxy)carbonyl)piperidin-1-yl)sulfonyl)carbamoyl)oxy)acetic acid (I-50-3) (18.32 mg, 52.30 μmol) and (S)-2-amino-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (30 mg, 52.30 μmol) were dissolved in DMF (1 mL), then added with 2-(7-azobenzotriazole)-N,N,N′,N′-tetramethyluronium hexafluorophosphate (23.85 mg, 62.76 μmol) and DIPEA (20.28 mg, 156.89 μmol), and stirred at room temperature for 1 hour. After the reaction was completed, the product was purified by flash column chromatography (C18, water/acetonitrile=1/2) and freeze-dried to obtain compound I-50-4 (40 mg, 44.15 μmol).

Its structural characterization data are as follows:

MS m/z (ESI): 906.4 [M+H]⁺

### Step 4

Allyl 1-((2-(((S)-1-(((S)-1-(((S)-1-((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)amino)-2-oxoethoxy)carbonyl)aminosulfonyl)piperidin-4-oate (I-50-4) (40 mg, 44.15 μmol) was dissolved in DMF (1 mL), added with tetra(triphenylphosphine) palladium (5.1 mg, 4.41 μmol), then added with 15 μL of formic acid and 30 μL of N-methylmorpholine, and stirred at room temperature for 1 hour under nitrogen protection. After the reaction was completed, the product was purified by flash column chromatography (C18, water/acetonitrile=3/1) and freeze-dried to obtain compound I-50-5 (20 mg, 23.10 μmol).

Its structural characterization data are as follows:

MS m/z (ESI): 867.4 [M+H]⁺

### Step 5

1-(N-((2-(((S)-1-(((S)-1-(((S)-9-Ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutane-2-yl)amino)-2-oxoethoxy)carbonyl)aminosulfonyl)piperidin-4-oic acid (I-50-5) (20 mg, 23.10 μmol) was dissolved in DMF (1 mL), and added with 1-(3-dimethylaminopropyl)-3-ethylcarbodiimide hydrochloride (27.72 mg, 144.36 μmol) and pentafluorophenol (15.94 mg, 86.61 μmol). After the reaction was completed, the reaction solution was directly purified by preparative high performance liquid chromatography and freeze-dried to obtain compound I-50 (12.53 mg, 12.02 mol).

Its structural characterization data are as follows:

MS m/z (ESI): 1033.4 [M+H]⁺

Its preparation method was as follows:

Chromatographic column: Waters Xbridge Prep C18 OBD (5 μm*19 mm*150 mm)

Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 32 |
| 4.00 | 10 | 90 | 32 |
| 24.00 | 90 | 10 | 32 |

### II. Preparation of Antibodies and Determination of Binding Activity

### Obtaining and Purification of Antibodies

According to the amino acid sequence of Trastuzumab (IMGT/mAb-DB ID: 97) and the amino acid sequence of Pertuzumab (IMGT/mAb-DB ID: 80) in the IMGT database, codon optimization was performed and the coding genes were synthesized, constructed into expression vectors, transfected into CHO cells and screened under pressure to construct stable expression cell lines. After expression, the supernatants were collected, and the corresponding antibodies Trastuzumab and Pertuzumab were purified by Protein A affinity filler.

III. Conjugation of Compounds Containing Cell Bioactive Molecules and Linkers with Antibodies

The antibodies Trastuzumab and Pertuzumab involved in the antibody-drug conjugates prepared in the following examples are Trastuzumab and Pertuzumab as described in the second section above.

The conjugation preparation of antibody-drug conjugate samples was as follows:

### 1. Preparation of Trastuzumab-E-5

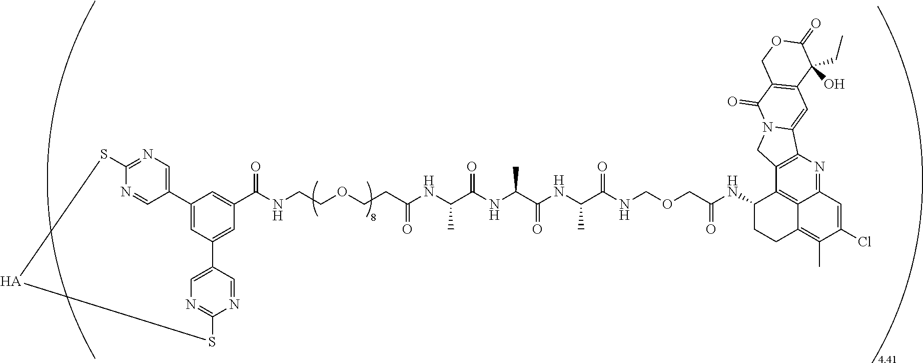

0.617 mL of Trastuzumab antibody (16.2 mg/mL) was taken, diluted with 30.86 μL of 20 mM PB+0.1M EDTA (pH 7.60), then adjusted to pH 7.60 with 1M Na₂HPO₄ solution, added with 19.83 mM TCEP (tris(2-carboxyethyl)phosphine, 19.1 μL, pH 7.60) solution, mixed well, and allowed to stand at room temperature for 1.5 hours. Then, E-5 (38.28 μL, 10 mM) that was in an amount 5.5 times the mole number of the antibody and dissolved in dimethyl sulfoxide solution was added, mixed well, and allowed to stand at room temperature for 18 hours; and then NAP-5 gel column (Cytiva) was used to replace the buffer with 20 mM histidine buffer solution at pH 6.0 to obtain an antibody-drug conjugate (i.e., Trastuzumab-E-5). The DAR value determined by mass spectrometry was 4.41.

### 2. Preparation of Trastuzumab-F-1

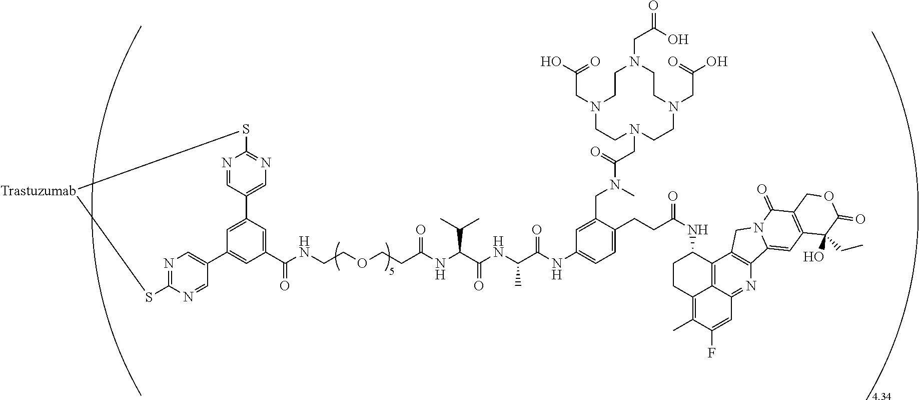

0.884 mL of Trastuzumab (14.7 mg/mL) was taken, diluted with 44 μL of 20 mM PB+0.1M EDTA (pH 7.60), then adjusted to pH 8.0 with 1M Na₂HPO₄ solution, added with 10 mM TCEP (tri(2-carboxyethyl)phosphine, 49.26 μL, pH 7.60) solution, mixed well, and allowed to stand at room temperature for 1.5 hours. Then F-1 (47.65 μL, 10 mM) that was in an amount 5 times the mole number of the antibody and dissolved in dimethyl sulfoxide solution was added slowly, mixed well, and allowed to stand at room temperature for 2 hours; and then NAP gel column (Cytiva) was used to replace the buffer with 20 mM histidine buffer solution at pH 6.0 to obtain an antibody-drug conjugate (i.e. Trastuzumab-F-1). The DAR value determined by mass spectrometry was 4.34.

### 3. Preparation of Trastuzumab-F-2

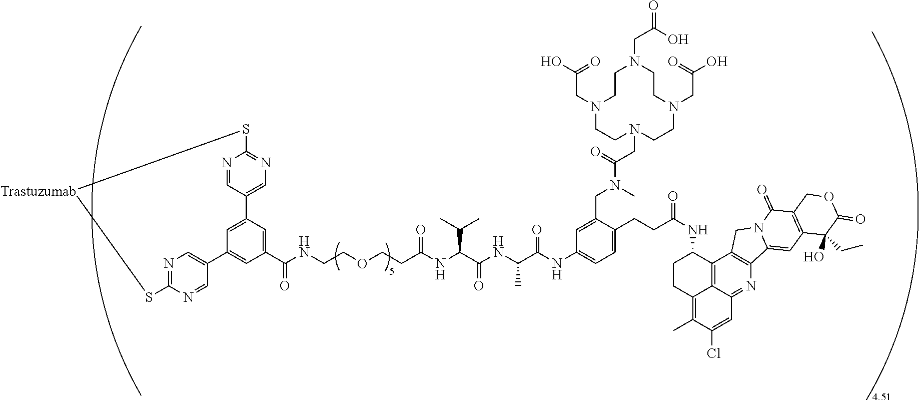

2.333 mL of Trastuzumab (15 mg/mL) was taken, diluted with 117 μL of 20 mM PB+0.1M EDTA (pH 7.60), then adjusted to pH 7.60 with 1M Na₂HPO₄ solution, added with 10 mM TCEP (tri(2-carboxyethyl)phosphine, 132.6 μL, pH 7.60) solution, mixed well, and allowed to stand at room temperature for 1.5 hours. Then F-2 (171.3 μL, 10 mM) that was in an amount 6.5 times the mole number of the antibody and dissolved in dimethyl sulfoxide solution was added, mixed well, and allowed to stand at room temperature for 18 hours; and then NAP gel column (Cytiva) was used to replace the buffer with 20 mM histidine buffer solution at pH 6.0 to obtain an antibody-drug conjugate (i.e. Trastuzumab-F-2). The DAR value determined by mass spectrometry was 4.51.

### 4. Preparation of Trastuzumab-F-4

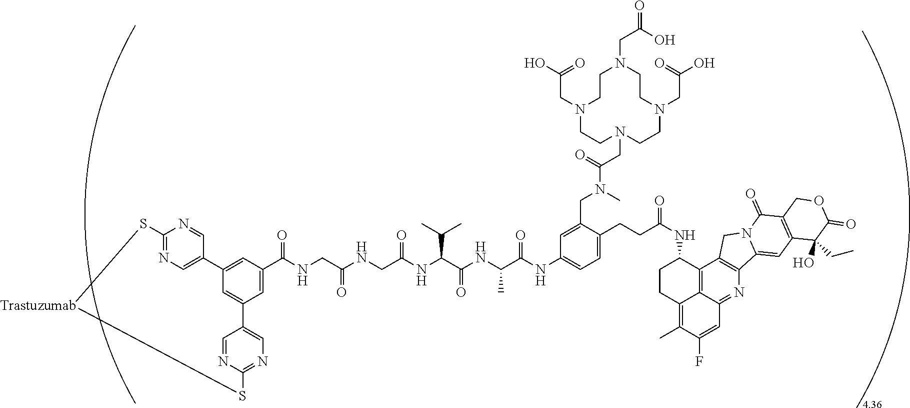

0.238 mL Trastuzumab (14.7 mg/mL) was taken, diluted with 12 μL of 20 mM PB+0.1M EDTA (pH 7.60), then adjusted to pH 7.6 with 1M Na₂HPO₄ solution, added with 10 mM TCEP (tri(2-carboxyethyl)phosphine, 13.26 μL, pH 7.60) solution, mixed well, and allowed to stand at room temperature for 1.5 hours. Then F-4 (12.18 μL, 10 mM) that was in an amount 5 times the mole number of the antibody and dissolved in dimethyl sulfoxide solution was added, mixed well, and allowed to stand at room temperature for 18 hours; and then NAP gel column (Cytiva) was used to replace the buffer with 20 mM histidine buffer solution at pH 6.0 to obtain an antibody-drug conjugate (i.e. Trastuzumab-F-4). The DAR value determined by mass spectrometry was 4.36.

### 5. Preparation of Trastuzumab-F-13

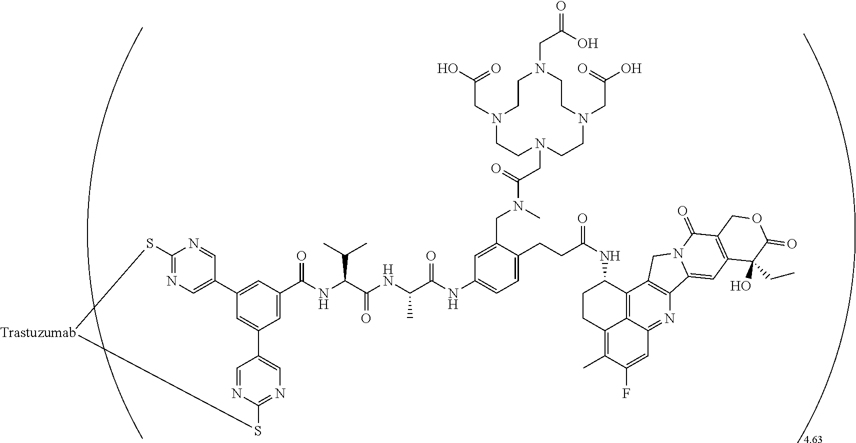

0.204 mL of Trastuzumab (14.7 mg/mL) was taken, diluted with 10 μL of 20 mM PB+0.1M EDTA (pH 7.60), then adjusted to pH 7.6 with 1M Na₂HPO₄ solution, added with 10 mM TCEP (tri(2-carboxyethyl)phosphine, 11.4 μL, pH 7.60) solution, mixed well, and allowed to stand at room temperature for 1.5 hours. Then F-13 (12.5 μL, 10 mM) that was in an amount 6 times the mole number of the antibody and dissolved in dimethyl sulfoxide solution was added, mixed well, and allowed to stand at room temperature for 18 hours; and then NAP gel column (Cytiva) was used to replace the buffer with 20 mM histidine buffer solution at pH 6.0 to obtain an antibody-drug conjugate (i.e. Trastuzumab-F-13). The DAR value determined by mass spectrometry was 4.63.

### 6. Preparation of Trastuzumab-G-1

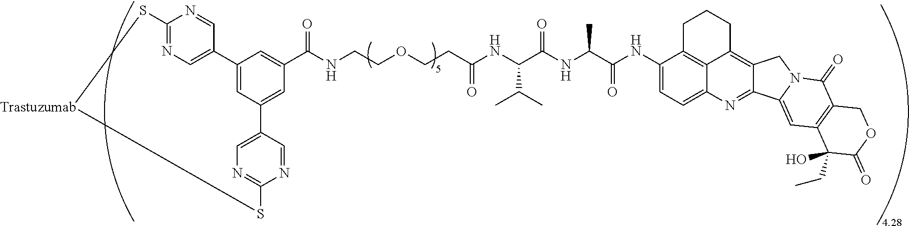

2.041 mL of Trastuzumab (14.7 mg/mL) was taken, diluted with 102 μL of 20 mM PB+0.1M EDTA (pH 7.60), then adjusted to pH 8.0 with 1M Na₂HPO₄ solution, added with 10 mM TCEP (tri(2-carboxyethyl)phosphine, 113.7 μL, pH 7.60) solution, mixed well, and allowed to stand at room temperature for 1.5 hours. Then G-1 (155.4 μL, 10 mM) that was in an amount 7.5 times the mole number of the antibody and dissolved in dimethyl sulfoxide was added, mixed well, and allowed to stand at room temperature for 18 hours; and then NAP gel column (Cytiva) was used to replace the buffer with 20 mM histidine buffer solution at pH 6.0 to obtain an antibody-drug conjugate (i.e. Trastuzumab-G-1). The DAR value determined by mass spectrometry was 4.28.

### 7. Preparation of Trastuzumab-G-2

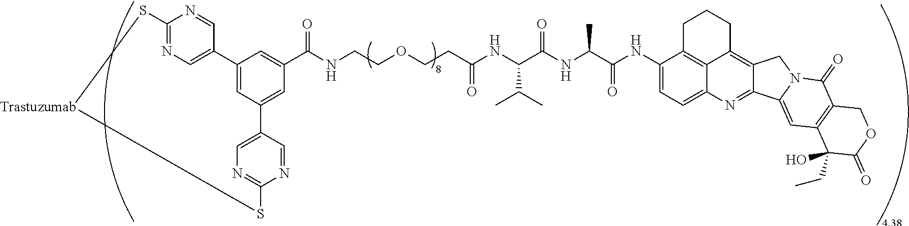

2.041 mL of Trastuzumab (14.7 mg/mL) was taken, diluted with 102 μL of 20 mM PB+0.1M EDTA (pH 7.60), then adjusted to pH 8.0 with 1M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then G-2 (126.2 μL, 10 mM) that was in an amount 6 times mole number of the antibody and dissolved in dimethyl sulfoxide was added, mixed well, and allowed to stand at room temperature for 18 hours; and then NAP gel column (Cytiva) was used to replace the buffer with 20 mM histidine buffer solution at pH 6.0 to obtain an antibody-drug conjugate (i.e., Trastuzumab-G-2). The DAR value determined by mass spectrometry was 4.38.

### 8. Preparation of Trastuzumab-G-3

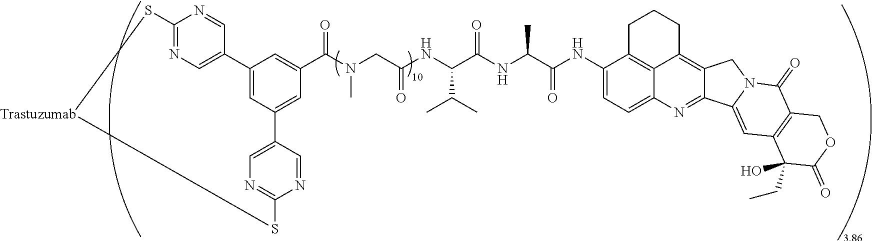

2.041 mL of Trastuzumab (14.7 mg/mL) was taken, diluted with 102 μL of 20 mM PB+0.1 M EDTA (pH 7.60), then adjusted to pH 8.0 with 1 M Na₂HPO₄ solution, added with 10 mM TCEP (tris(2-carboxyethyl)phosphine, 113.7 μL, pH 7.60) solution, mixed well, and allowed to stand at room temperature for 1.5 hours. Then G-3 (125.2 μL, 10 mM) that was in an amount 6 times the mole number of the antibody and dissolved in dimethyl sulfoxide solution was added, mixed well, and allowed to stand at room temperature for 18 hours; and then NAP gel column (Cytiva) was used to replace the buffer with 20 mM histidine buffer solution at pH 6.0 to obtain an antibody-drug conjugate (i.e. Trastuzumab-G-3). The DAR value determined by mass spectrometry was 3.86.

### 9. Preparation of Trastuzumab-G-4

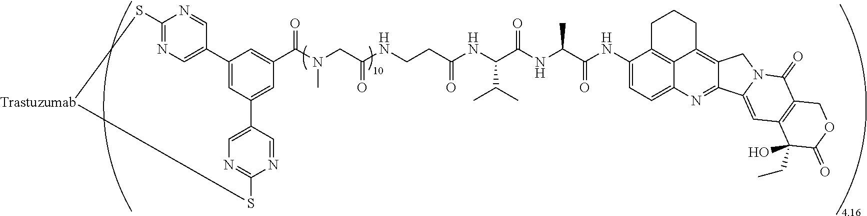

2.041 mL of Trastuzumab (14.7 mg/mL) was taken, diluted with 102 μL of 20 mM PB+0.1M EDTA (pH 7.60), then adjusted to pH 7.6 with 1M Na₂HPO₄ solution, added with 10 mM TCEP (tris (2-carboxyethyl) phosphine, 113.7 μL, pH 7.60) solution, mixed well, and allowed to stand at room temperature for 1.5 hours. Then G-4 (125.3 μL, 10 mM) that was in an amount 6 times the mole number of the antibody and dissolved in dimethyl sulfoxide solution was added, mixed well, and allowed to stand at room temperature for 18 hours; and then NAP gel column (Cytiva) was used to replace the buffer with 20 mM histidine buffer solution at pH 6.0 to obtain an antibody-drug conjugate (i.e. Trastuzumab-G-4). The DAR value determined by mass spectrometry was 4.16.

### 10. Preparation of Trastuzumab-G-5

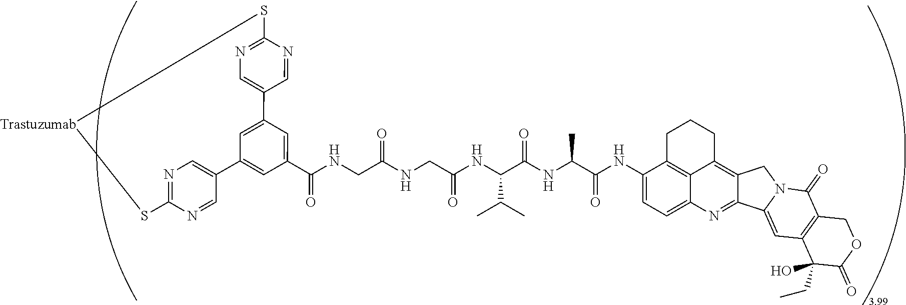

0.884 mL of Trastuzumab (14.7 mg/mL) was taken, diluted with 44.2 μL of 20 mM PB+0.1M EDTA (pH 7.60), then adjusted to pH 7.6 with 1M Na₂HPO₄ solution, added with 10 mM TCEP (tris(2-carboxyethyl)phosphine, 49.3 μL, pH 7.60) solution, mixed well, and allowed to stand at room temperature for 1.5 hours. Then G-5 (54 μL, 10 mM) that was in an amount 6 times the mole number of the antibody and dissolved in dimethyl sulfoxide solution was added, mixed well, and allowed to stand at room temperature for 18 hours; and then NAP gel column (Cytiva) was used to replace the buffer with 20 mM histidine buffer solution at pH 6.0 to obtain an antibody-drug conjugate (i.e. Trastuzumab-G-5). The DAR value determined by mass spectrometry was 3.99.

### 11. Preparation of Trastuzumab-I-50

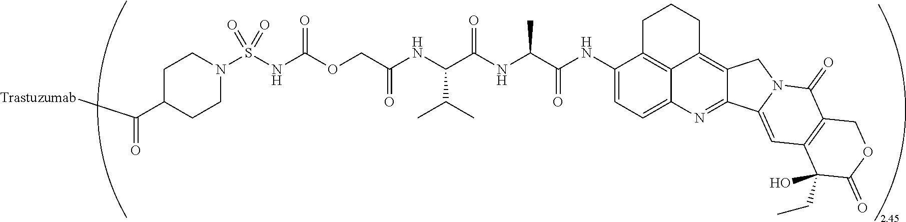

2.04 mL of Trastuzumab (14.7 mg/mL) was taken, adjusted to pH 7.40 with 1M Na₂HPO₄ solution, added with 1-50 (146 μL, 10 mM, equivalent to 7 times the mole number of the antibody) dissolved in dimethyl sulfoxide, mixed well, and allowed to stand at room temperature for 18 hours; and then NAP-5 gel column (Cytiva) was used to replace the buffer with 20 mM histidine buffer solution at pH 6.0 to obtain an antibody-drug conjugate (i.e., Trastuzumab-I-50). The average load (DAR value) determined by mass spectrometry was 2.45.

### IV. Evaluation of Tumor Growth Inhibitory Effects of Antibody-Drug Conjugates in Mouse Subcutaneous Transplanted Tumor Model

The preparations containing ADCs were respectively administered through tail vein injection to the mouse CDX model subcutaneously transplanted with human gastric cancer cell NCI-N87, the changes in tumor volume and animal body weight were measured twice weekly, and the tumor inhibition efficacy of the ADCs on tumor-bearing mice was calculated.

### Test Drug

Drug name, source, preparation method: The ADCs at appropriate amounts were taken; based on the administration dose of 1 mg/kg, 0.9 NaCl injection was used to dilute the stock solution. 0.9 NaCl injection was used as the solvent control (Vehicle).

### Experimental Animal and Cell Line

Balb/c Nude mice (Chengdu Yaokang Biotechnology Co., Ltd., production license number: SCXK (Sichuan) 2020-0034, animal certificate number: 511214900025102)

Human gastric cancer cells NCI-N87 (ATCC)

### Experimental Grouping and Evaluation Method

The tumor-bearing mice with tumors having an average volume of about 150 mm³ were selected, and randomly divided into groups (the number of groups was determined according to the number of samples). According to the groups, 0.9 NaCl injection (hereinafter referred to as solvent control, Vehicle) and ADCs were respectively administered. The frequencies of administration were shown in the specific examples. The administration model was tail vein injection, and the administration volume was 10 mL/kg. After administration, the tumor diameters were measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

The tumor growth inhibition rate TGI (%) was calculated using the following formula to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ) ] * 100 \%$$

$$or$$

$${V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADC of the present invention:

$$T / C = ( {V}_{T end} / {V}_{T 0} ) / ( {V}_{C end} / {V}_{C 0} ) .$$

### Detection of Efficacy of Anti-Human Her2 Antibody-Drug Conjugates in NCI-N87 Model

NCI-N87 cells were cultured in RPMI 1640 medium containing 10% fetal bovine serum at 37° C. and 5% CO₂. NCI-N87 cells in the exponential growth phase were collected, resuspended in PBS to a suitable concentration, and inoculated subcutaneously in female Balb/c-Nude mice to establish a gastric cancer model. When the average tumor volume was about 150 mm³, the mice were randomly divided into groups according to the tumor size, including in order: a vehicle control group (i.e., negative control, Vehicle group), and a Trastuzumab-F-2 at 1 mg/kg group, a Trastuzumab-G-1 at 1 mg/kg group, and a control DS8201 at 1 mg/kg group (Note: DS8201 was an ADC targeting human HER2 that was developed by Daiichi Sankyo, and the sample used in this experiment was prepared internally by Kelun-Biotech). Each group adopted tail vein injection (i.v.), and administration was performed on Day 0, Day 7 and Day 14, three times in total.

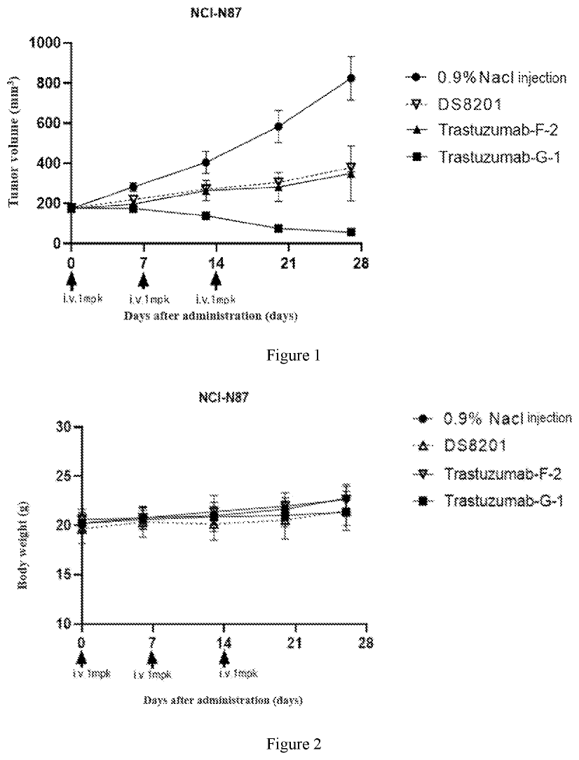

The ADCs of the present invention exhibit significant inhibitory effects on the tumor growth in the NCI-N87 gastric cancer xenograft model. Compared with the Vehicle group, the tumor growth inhibition rate (TGI) values of the Trastuzumab-F-2 at 1 mg/kg group, Trastuzumab-G-1 at 1 mg/kg group of the present invention, and the control DS8201 at 1 mg/kg group are 72.86%, 167.97%, 69.02%, respectively. Until Day 27, there was no animal death and significant animal weight loss in the treatment groups, and no obvious drug toxicity reaction was observed. The mice had good tolerance to the ADCs of the present invention during the treatment period. The specific results are shown in Table 1, and FIGS. 1 and 2.

TABLE 1
Human gastric cancer cell NCI-N87 CDX model

| | | Day 27 | | | |
|---|---|---|---|---|---|
| | Dose | Tumor | | | P value |
| | (mg/ | volume (mm³) | TGI | T/C | (vs. |
| Group | kg) | (x̅ ± SEM) | (%) | (%) | Vehicle) |
| Vehicle | — | 823.72 ± 108.03 | — | — | — |
| DS8201 | 1 | 378.76 ± 21.93 | 69.02 | 45.27 | <0.05 |
| Trastuzumab-F-2 | 1 | 349.90 ± 136.87 | 72.86 | 42.77 | <0.05 |
| Trastuzumab-G-1 | 1 | 56.85 ± 9.81 | 167.97 | 6.81 | <0.001 |

Note:
TGI is the tumor growth inhibition rate, and T/C is the relative tumor proliferation rate, the same below.

### V. Evaluation of Tumor Growth Inhibitory Effects of Antibody-Drug Conjugates in Mouse Subcutaneous Transplanted Tumor Model

The preparations containing ADCs were respectively administered through tail vein injection to the mouse CDX model subcutaneously transplanted with human gastric cancer cell NCI-N87, the changes in tumor volume and animal body weight were measured twice weekly, and the tumor inhibition efficacy of the ADCs on tumor-bearing mice was calculated.

### Test Drug

Drug name, source, preparation method: The ADCs at appropriate amounts were taken; based on the administration doses of 1 mg/kg and 2 mg/kg, 0.9 NaCl injection was used to dilute the stock solution. 0.9 NaCl injection was used as the solvent control (Vehicle).

### Experimental Animal and Cell Line

Balb/c Nude mice (Chengdu Yaokang Biotechnology Co., Ltd., production license number: SCXK (Sichuan) 2020-0034, animal certificate number: 511214900026661)

Human gastric cancer cells NCI-N87 (ATCC)

### Experimental Grouping and Evaluation Method

The tumor-bearing mice with tumors having an average volume of about 150 mm³ were selected, and randomly divided into groups (the number of groups was determined according to the number of samples). According to the groups, 0.9 NaCl injection (hereinafter referred to as solvent control, Vehicle) and ADCs were respectively administered. The frequencies of administration were shown in the specific examples. The administration model was tail vein injection, and the administration volume was 10 mL/kg. After administration, the tumor diameters were measured with a vernier caliper twice a week, and the tumor volume was calculated according to the following calculation formula: V=0.5 a×b², wherein a and b represented the long diameter and short diameter of the tumor, respectively. The death of animals was observed and recorded every day.

The tumor growth inhibition rate TGI (%) was calculated using the following formula to evaluate the tumor inhibition efficacy of the ADCs of the present invention:

$${V}_{T end} > {V}_{T 0} ,$$

$$TGI ( \% ) = [ 1 - ( {V}_{T end} - {V}_{T 0} ) / ( {V}_{C end} - {V}_{C 0} ) ] * 100 \%$$

$$or$$

$${V}_{T end} \leq {V}_{T 0} ,$$

$$TGI ( \% ) = [ 1 - ( {V}_{T end} - {V}_{T 0} ) / {V}_{T 0} ] * 100 \% .$$

wherein,
V_{T end}: mean of tumor volume of the treatment group at the end of experiment
V_{T0}: mean of tumor volume of the treatment group at the beginning of administration
V_{C end}: mean of tumor volume of the negative control group at the end of experiment
V_{C0}: mean of tumor volume of the negative control group at the beginning of administration

The following formula was used to calculate the tumor relative proliferation rate T/C (%), which was used to evaluate the tumor inhibition efficacy of the ADC of the present invention:

$$T / C = ( {V}_{T end} / {V}_{T 0} ) / ( {V}_{C end} / {V}_{C 0} ) .$$

### Detection of Efficacy of Anti-Human Her2 Antibody-Drug Conjugates in NCI-N87 Model

NCI-N87 cells were cultured in RPMI 1640 medium containing 10% fetal bovine serum at 37° C. and 5% CO₂. NCI-N87 cells in the exponential growth phase were collected, resuspended in PBS to a suitable concentration, and inoculated subcutaneously in female Balb/c-Nude mice to establish a gastric cancer model. When the average tumor volume was about 150 mm³, the mice were randomly divided into groups according to the tumor size, including in order: a vehicle control group (i.e., negative control, Vehicle group), and a Trastuzumab-F-1 at 2 mg/kg group, a Trastuzumab-G-5 at 1 mg/kg group, a Trastuzumab-G-2 at 1 mg/kg group, a Trastuzumab-G-3 at 1 mg/kg group, a Trastuzumab-G-4 at 1 mg/kg group, and a control DS8201 at 1 mg/kg group (Note: DS8201 was an ADC targeting human HER2 that was developed by Daiichi Sankyo, and the sample used in this experiment was prepared internally by Kelun-Biotech). Each group adopted tail vein injection (i.v.), and administration was performed on Day 0, Day 7 and Day 14, three times in total.

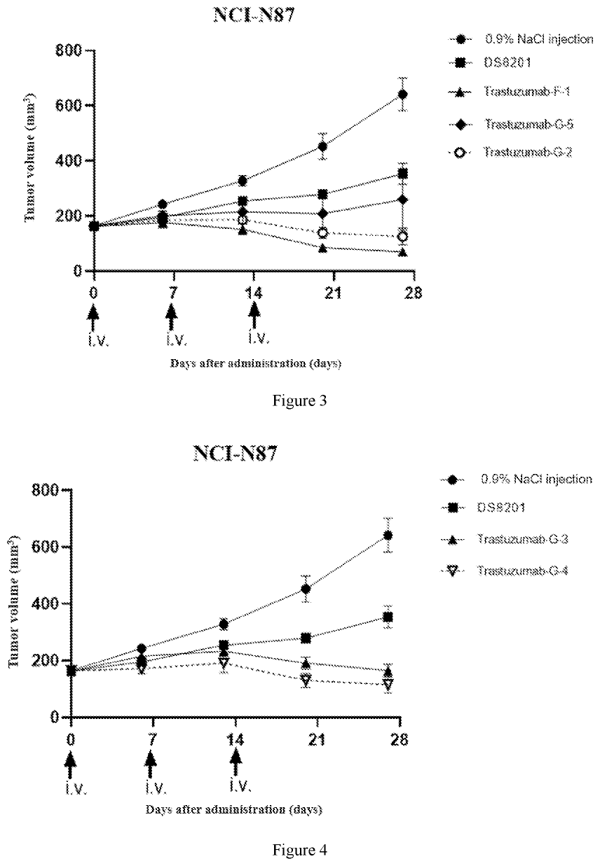

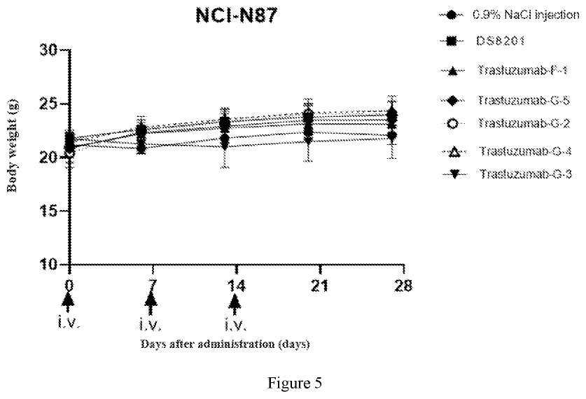

The ADCs of the present invention exhibit significant inhibitory effects on the tumor growth in the NCI-N87 gastric cancer xenograft model. Compared with the Vehicle group, the tumor growth inhibition rate (TGI) values of the Trastuzumab-F-1 at 2 mg/kg group, Trastuzumab-G-5 at 1 mg/kg group, Trastuzumab-G-2 at 1 mg/kg group, Trastuzumab-G-3 at 1 mg/kg group, Trastuzumab-G-4 at 1 mg/kg group of the present invention, and the control DS8201 at 1 mg/kg group are 156.31%, 80.06%, 122.21%, 99.55%, 130.19% and 60.21%, respectively. Until Day 27, there was no animal death and significant animal weight loss in the treatment groups, and no obvious drug toxicity reaction was observed. The mice had good tolerance to the ADCs of the present invention during the treatment period. The specific results are shown in Table 2, and FIGS. 3, 4 and 5.

TABLE 2
Human gastric cancer cell NCI-N87 CDX model

| | | Day 27 | | | |
|---|---|---|---|---|---|
| | Dose | Tumor | | | P value |
| | (mg/ | volume (mm³) | TGI | T/C | (vs. |
| Group | kg) | (x̅ ± SEM) | (%) | (%) | Vehicle) |
| Vehicle | — | 641.88 ± 59.85 | — | — | — |
| DS8201 | 1 | 353.38 ± 38.06 | 60.21 | 55.46 | <0.01 |
| Trastuzumab-F-1 | 2 | 71.28 ± 14.81 | 156.31 | 11.21 | <0.001 |
| Trastuzumab-G-5 | 1 | 260.05 ± 111.38 | 80.06 | 40.47 | <0.05 |
| Trastuzumab-G-2 | 1 | 126.58 ± 30.80 | 122.21 | 19.96 | <0.001 |
| Trastuzumab-G-3 | 1 | 165.03 ± 21.72 | 99.55 | 26.00 | <0.001 |
| Trastuzumab-G-4 | 1 | 114.68 ± 29.68 | 130.19 | 17.92 | <0.001 |

Note:
TGI is the tumor growth inhibition rate, and T/C is the relative tumor proliferation rate.

Although the specific embodiments of the present invention have been described in detail, those skilled in the art will understand that various modifications and substitutions can be made to those details based on all the teachings disclosed, and these changes are all within the scope of protection of the present invention. The full scope of the invention is given by the appended claims and any equivalents thereof.

1. An antibody-drug conjugate, which has a structure represented by the formula Ab-[M-L-E-D]ₓ, wherein:
Ab represents an antibody or antigen-binding fragment thereof capable of specifically binding to epidermal growth factor receptor 2 (Her2), a member of the ErbB family of receptor tyrosine kinases:
M represents a joint connecting to the antibody or antigen-binding fragment thereof;
L represents a structural fragment connecting M and E;
E represents a structural fragment connecting L and D;
D represents a cytotoxic drug fragment;
x is 1 to 10.

2. The antibody-drug conjugate according to claim 1, wherein Mis selected from the group consisting of the following substituted or unsubstituted structural fragments: 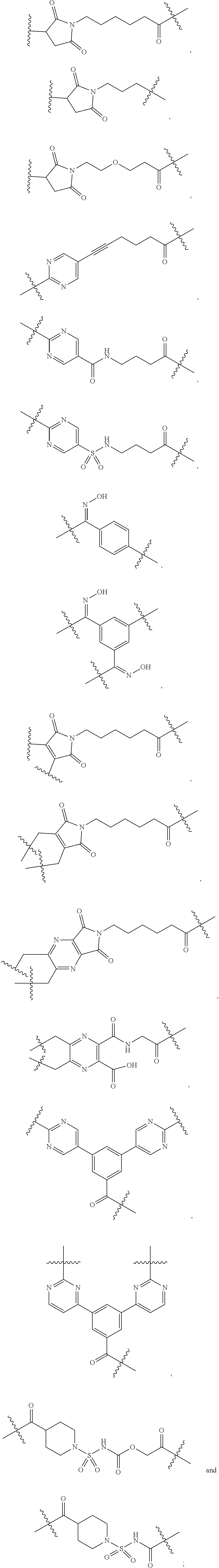
preferably, M is selected from the group consisting of the following substituted or unsubstituted structural fragments: 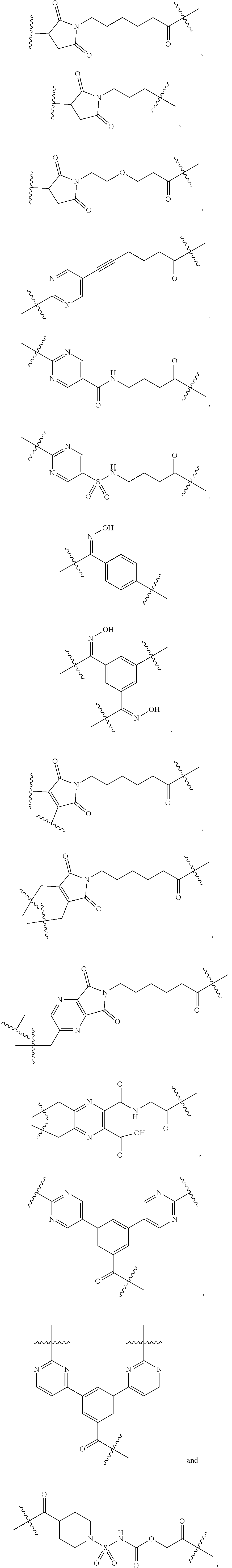
preferably, M is selected from the group consisting of the following substituted or unsubstituted structural fragments: 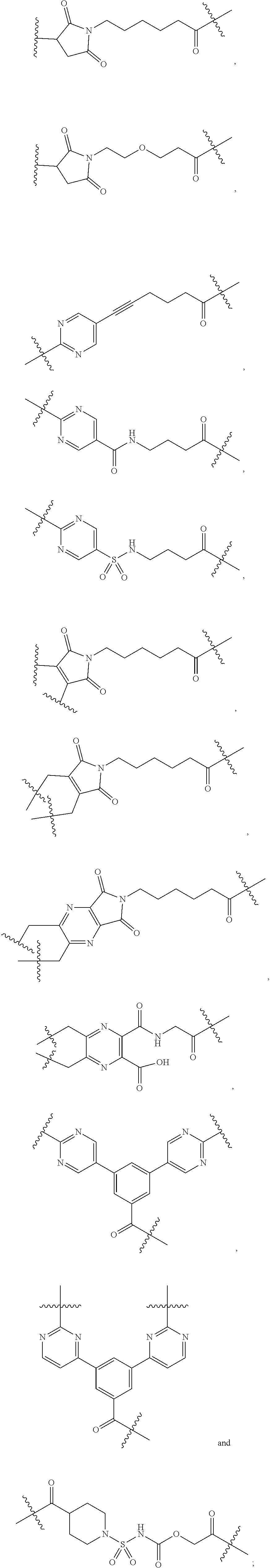
preferably, M is selected from the group consisting of the following substituted or unsubstituted structural fragments: 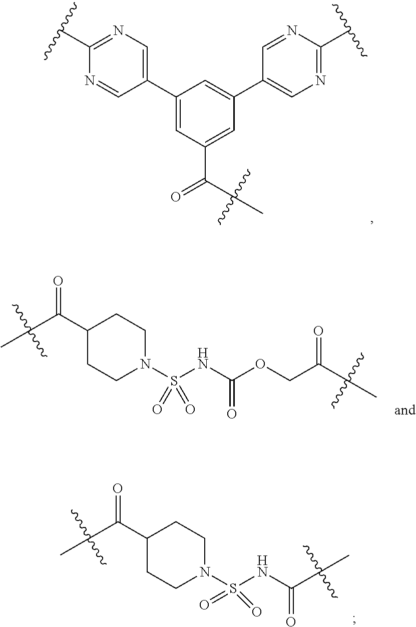 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1 ; *C(=O)NS(=O)(=O)N1CCC(C(*)=O)CC1]
preferably, M is selected from the group consisting of the following substituted or unsubstituted structural fragments: 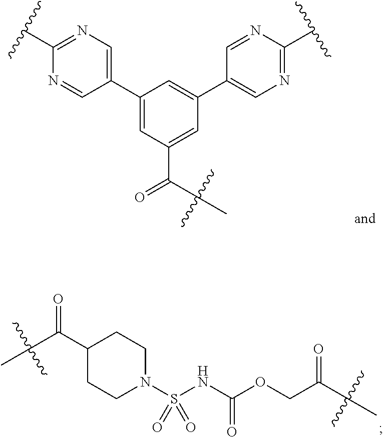 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1]
preferably, M is selected from the group consisting of the following substituted or unsubstituted structural fragments: 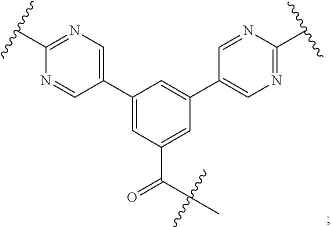 [SMILES: *C(=O)c1cc(-c2cnc(*)nc2)cc(-c2cnc(*)nc2)c1]
or, M is selected from the group consisting of the following substituted or unsubstituted structural fragments: 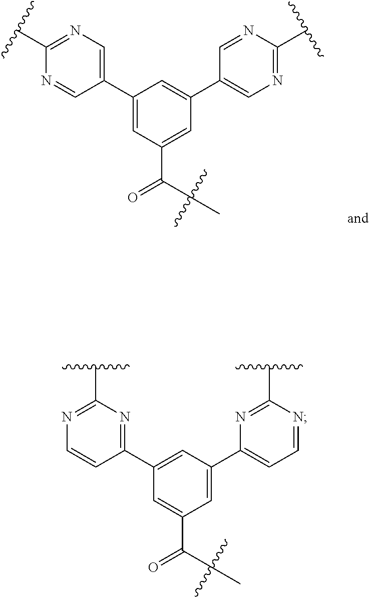 [SMILES: *C(=O)c1cc(-c2cnc(*)nc2)cc(-c2cnc(*)nc2)c1 ; *C(=O)c1cc(-c2ccnc(*)n2)cc(-c2ccnc(*)n2)c1]
or, M is selected from the group consisting of the following substituted or unsubstituted structural fragments:

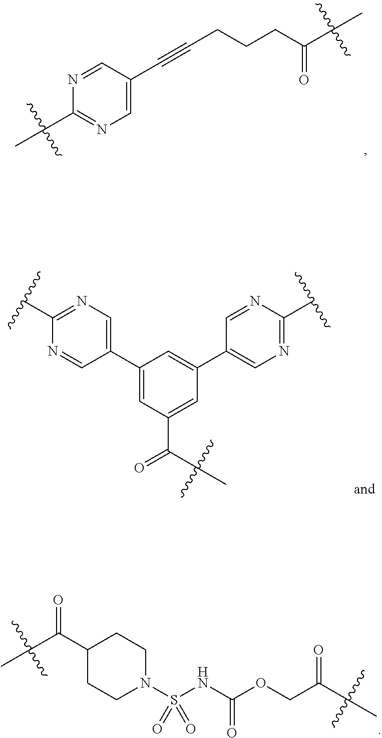 [SMILES: *C(=O)CCCC#Cc1cnc(*)nc1 ; *C(=O)c1cc(-c2cnc(*)nc2)cc(-c2cnc(*)nc2)c1 ; *C(=O)COC(=O)NS(=O)(=O)N1CCC(C(*)=O)CC1]

3. (canceled)

4. The antibody-drug conjugate according to claim 1, wherein L is selected from the group consisting of the substituted or unsubstituted structural fragments composed of one or more of the following groups: C₁₋₆ alkylene, 6- to 10-membered aryl, 5- to 6-membered heteroaryl, 9- to 12-membered nitrogen-containing heterocyclyl, —N(R′)—, —NH(R′), —N(R′)₂, —NHCH(R′)—C(═O)—, carbonyl group, —O—, natural amino acids or unnatural amino acids or analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), Glu(R′), Lys(R′), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly), 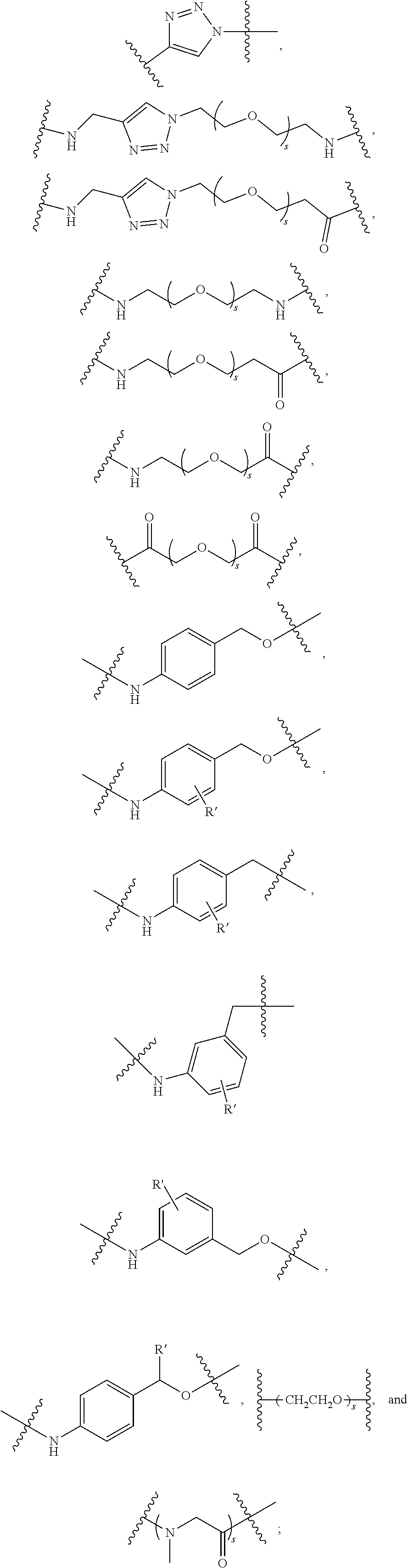
wherein R′ is composed of one or more of the following groups, including but not limited to hydrogen, C₁₋₆ alkyl, C₁₋₆ alkylene, amino, hydroxyl, carboxyl, acyl, —O—, —C₁₋₆ alkylene-CO₂H, —C₁₋₆ alkylene-SO₃H, —SO₃H, —PO₃H₂, —C₁₋₆ alkylene-NHC₁₋₆ alkyl, —C₁₋₆ alkylene-N(C₁₋₆ alkyl)₂, —C₁₋₆ alkylene-heterocycle, —CH₂N(C₁₋₆ alkyl)-C(═O)C₁₋₆ alkylene-heterocycle, —CH₂NH—SO₃H, —CH₂N(C₁₋₆ alkyl)-SO₃H, —CH₂NHC₁₋₆ alkylene-SO₃H, —CH₂N(C₁₋₆ alkyl)C₁₋₆ alkylene-SO₃H, —CH₂N(C₁₋₆ alkylene-SO₃H)₂, —CH₂N⁺(C₁₋₆ alkylene-SO₃H)₃, —CH₂N⁺(C₁₋₆ alkyl)₂-C₁₋₆ alkylene-SO₃H, —CH₂N(C₁₋₆ alkyl)-C(═O)C₁₋₆ alkylene-N⁺(C₁₋₆ alkylene-SO₃H)₃, —CH₂NH—C(═O)C₁₋₆ alkylene-N⁺(C₁₋₆ alkylene-SO₃H)₃, —CH₂N(C₁₋₆ alkyl)-C(═O)C₁₋₆ alkylene-N⁺(C₁₋₆ alkyl)₃, —CH₂NH—C(═O)C₁₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₂-CH₂CO₂H, —CH₂N(C₁₋₆ alkyl)-C₁₋₆ alkylene-CO₂H, —CH₂N⁺(C₁₋₆ alkyl)₂-C₁₋₆ alkylene-CO₂H, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—(OCH₂CH₂)ᵣ—OC₁₋₆ alkyl, —(CH₂N(Me)-C(═O))ᵣ—C₁₋₆ alkyl, polyethylene glycol fragment containing 1 to 10 EO units (i.e. —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-a-propionyl), NOTA (1,4,7-triazacyclononane-N,N′,N″-triacetic acid residue), —C₁₋₆ alkylene-N(C₁₋₆ alkyl)-DOTA, —C₁₋₆ alkyl-N(C₁₋₆ alkyl)-DOTAGA, or —C₁₋₆ alkyl-N(C₁₋₆ alkyl)-NOTA, wherein r is an integer selected from 1 to 20, preferably r is an integer selected from 1 to 15, such as an integer of 1 to 12, 3 to 12, 1 to 10, 1 to 8, 3 to 8, 1 to 6, 1 to 4, 1 to 2, for example, r is 1, 2, 3, 4, 5, 6, 7, 8, 9, 10, 11, 12, 13, 14 or 15; s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15;
preferably, L is selected from the group consisting of the substituted or unsubstituted structural fragments composed of one or more of the following groups: 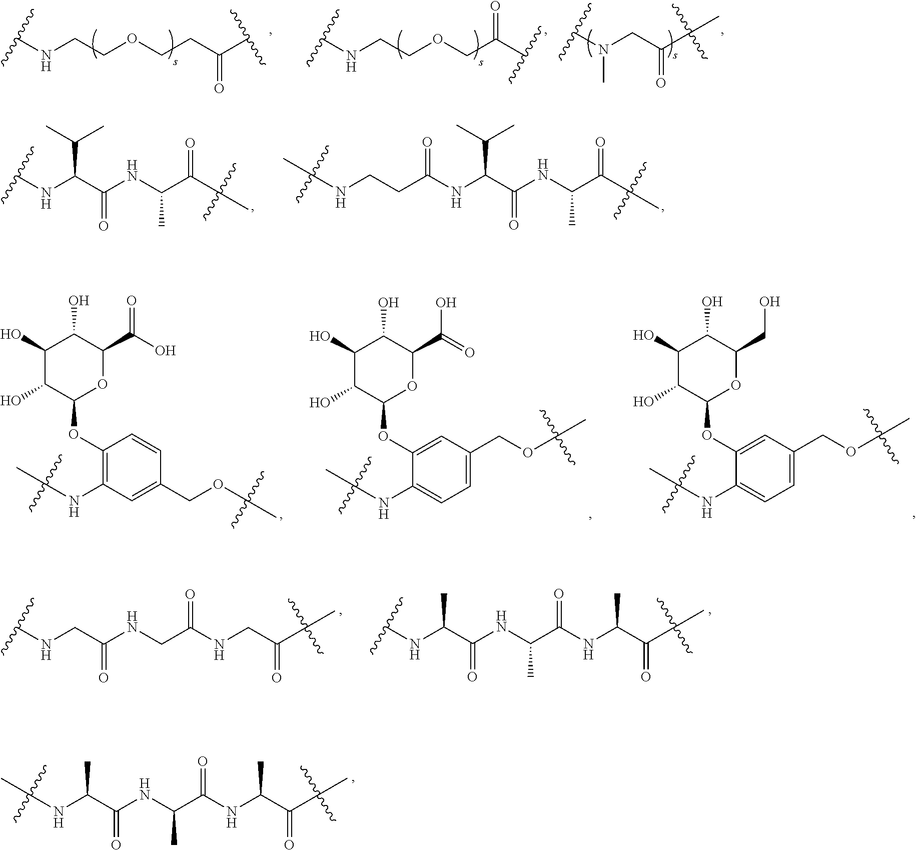 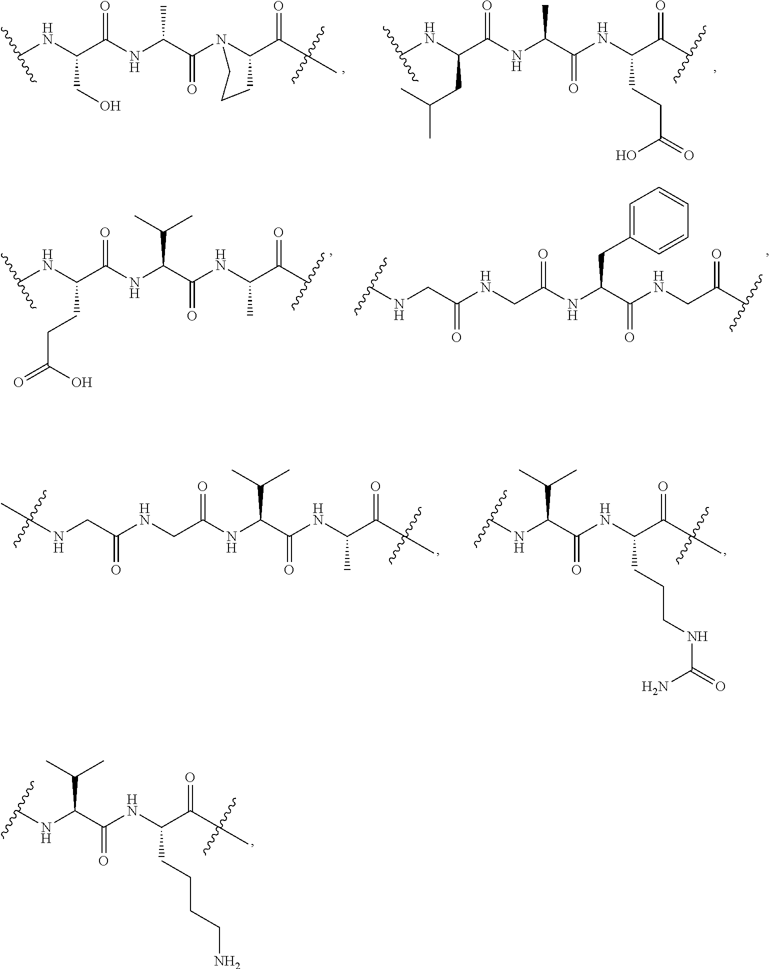 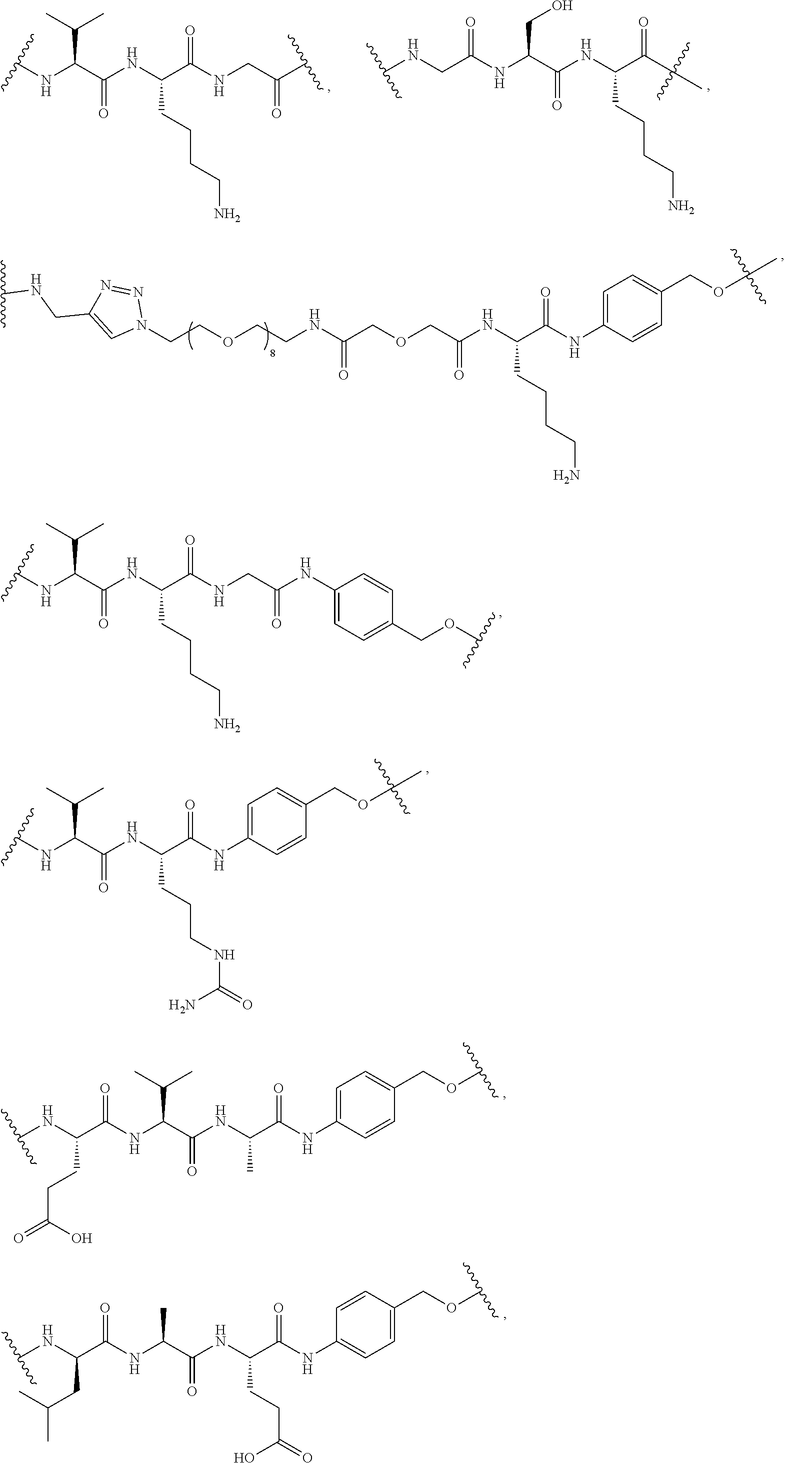 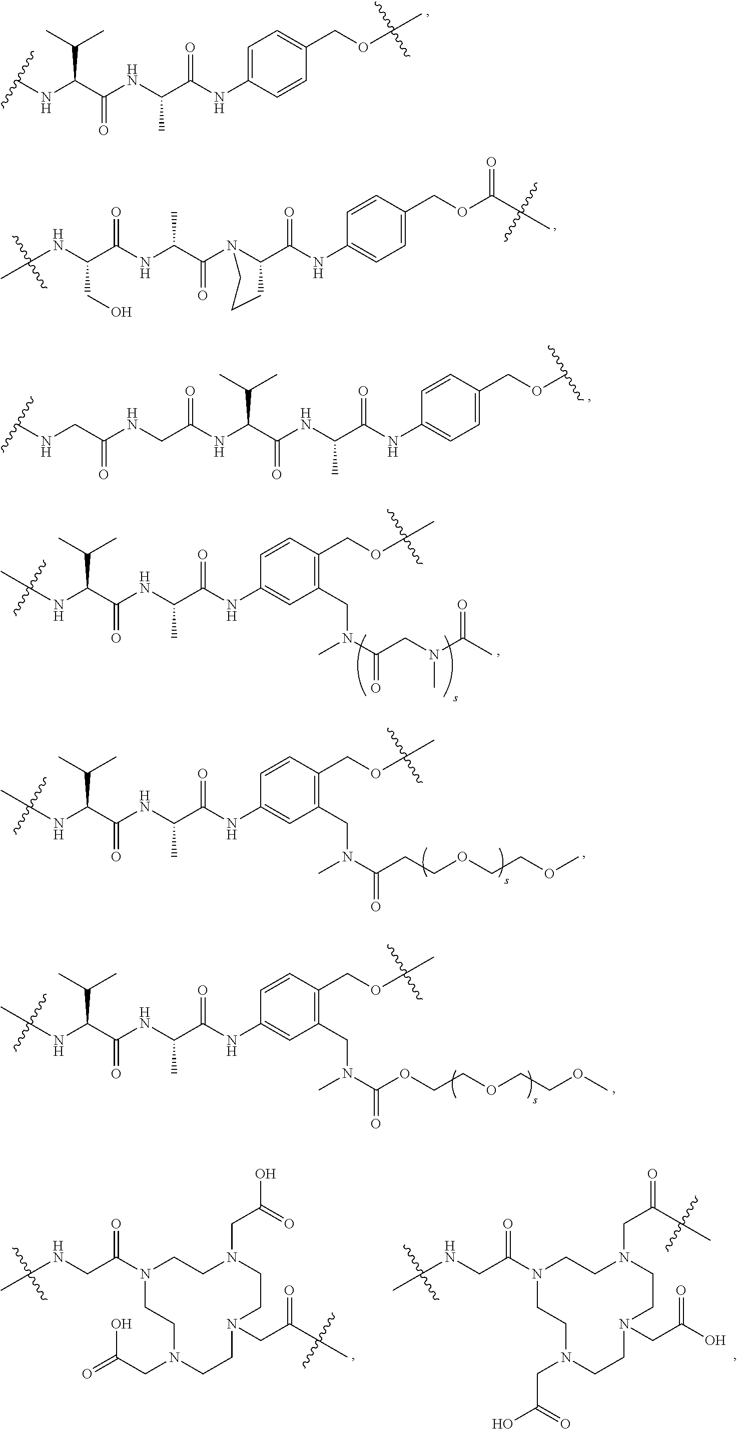 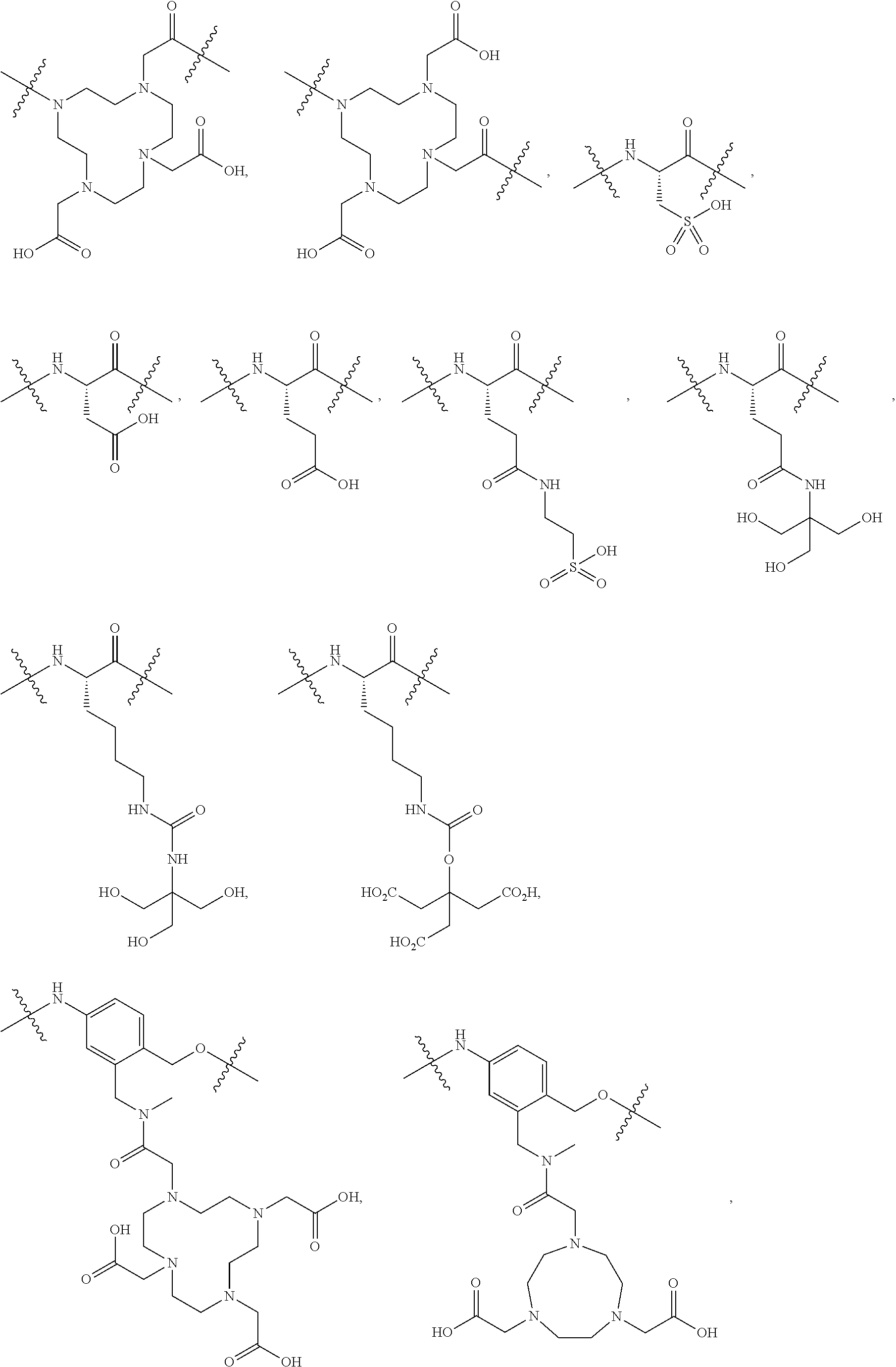 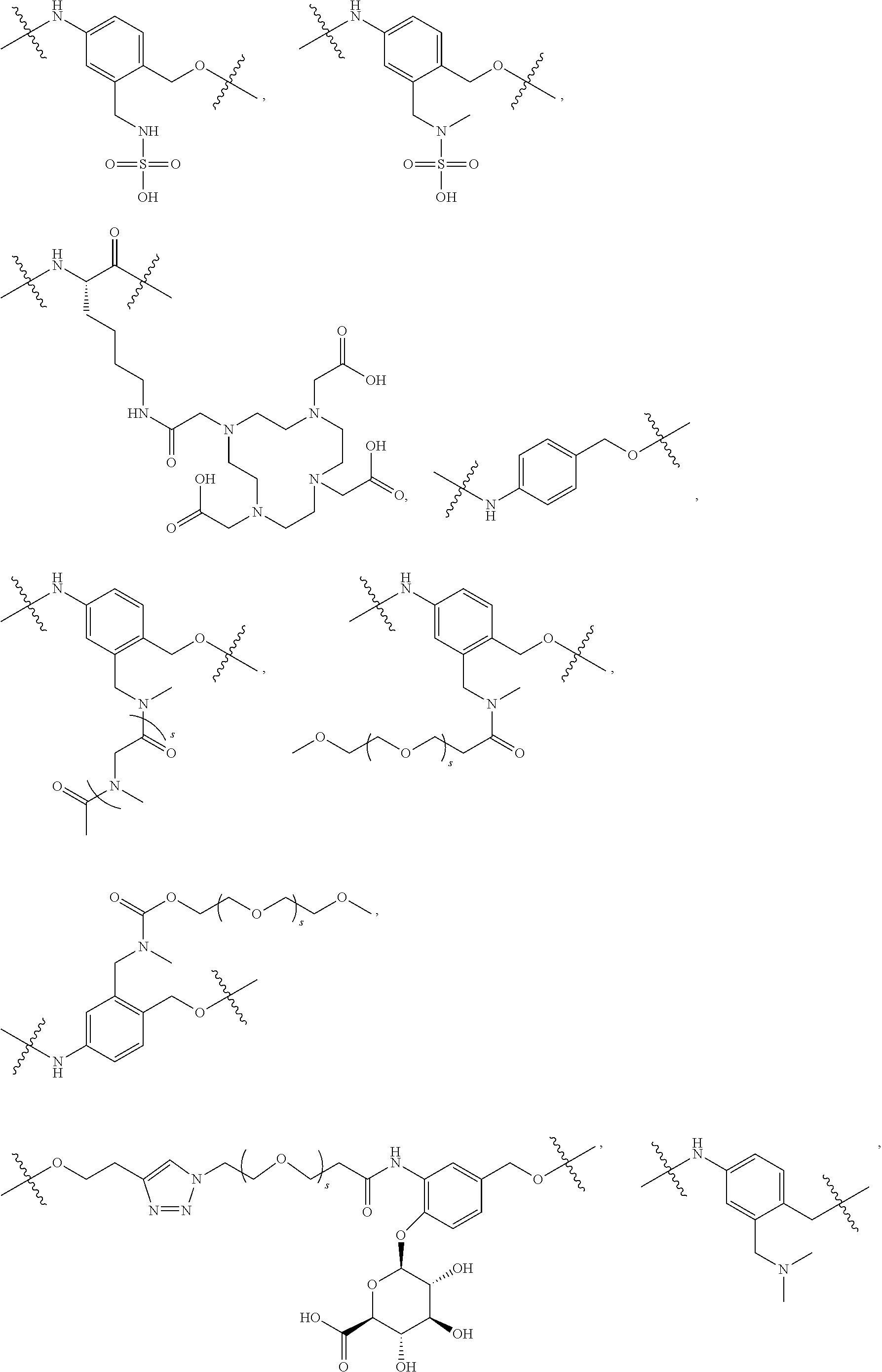 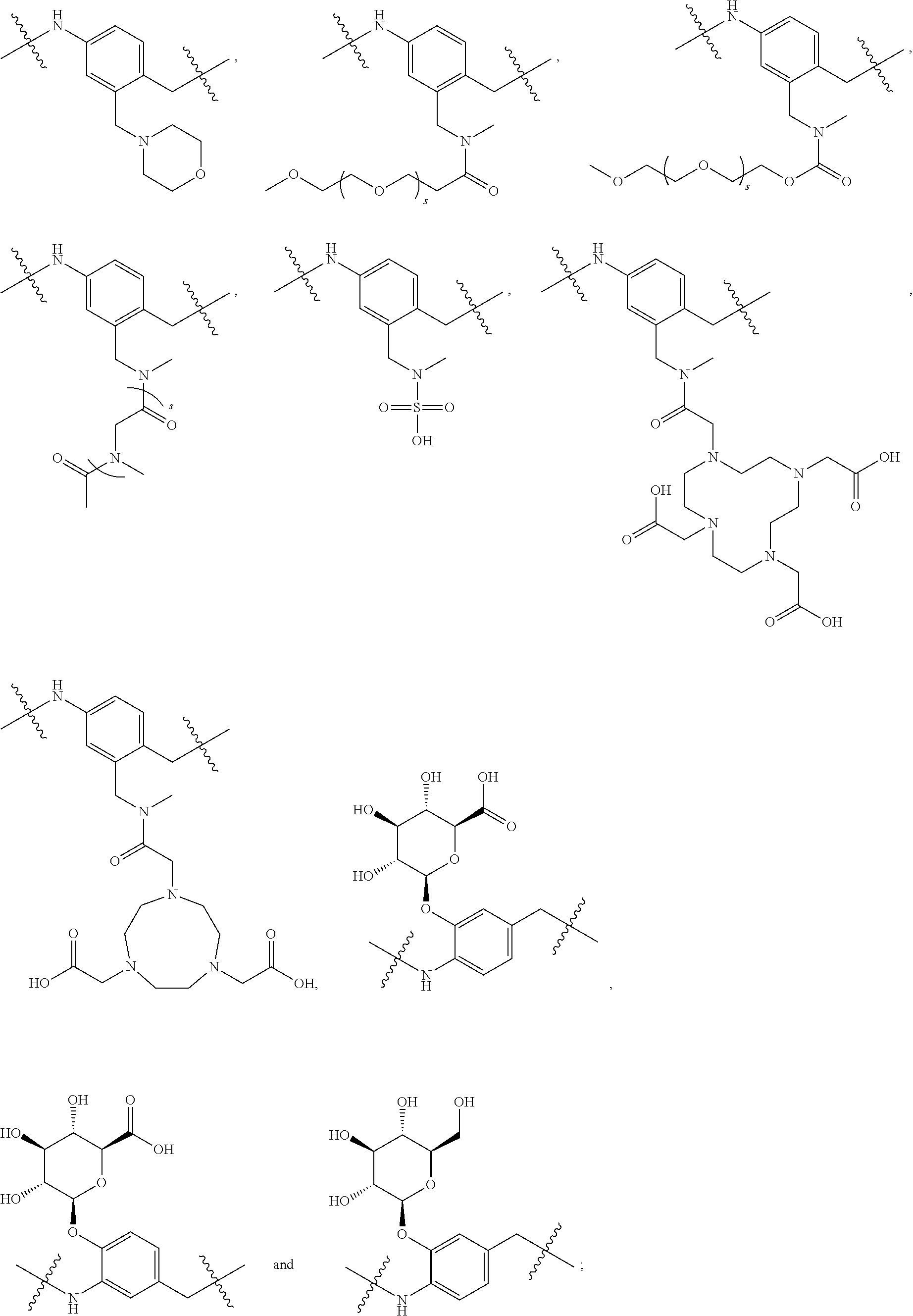
s is an integer selected from 1 to 20, preferably, s is an integer selected from 1 to 15, such as an integer of 1 to 12, 3 to 12, for example, s is 1, 2, 3, 4, 5, 6, 7, 8, 9, 10, 11, 12, 13, 14 or 15;
preferably, L is selected from the group consisting of the substituted or unsubstituted structural fragments composed of one or more of the following groups: 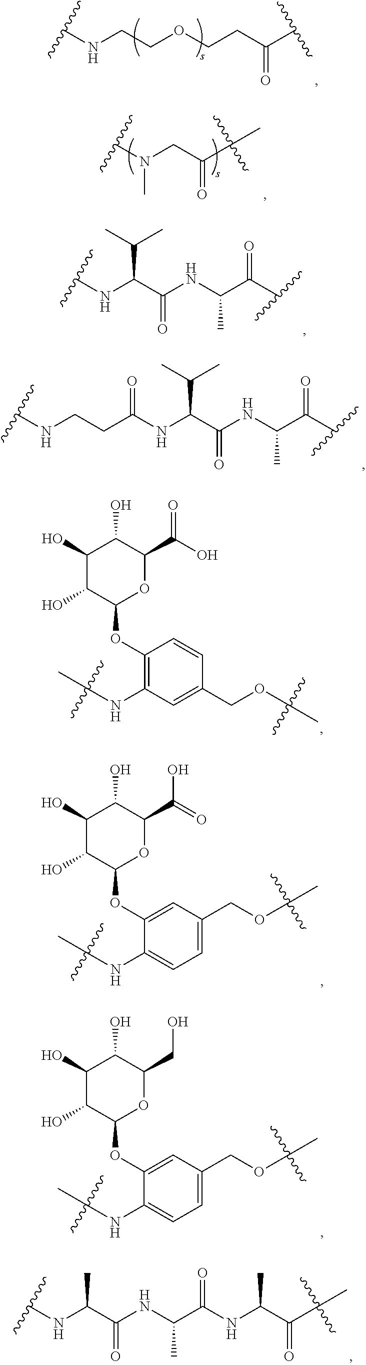 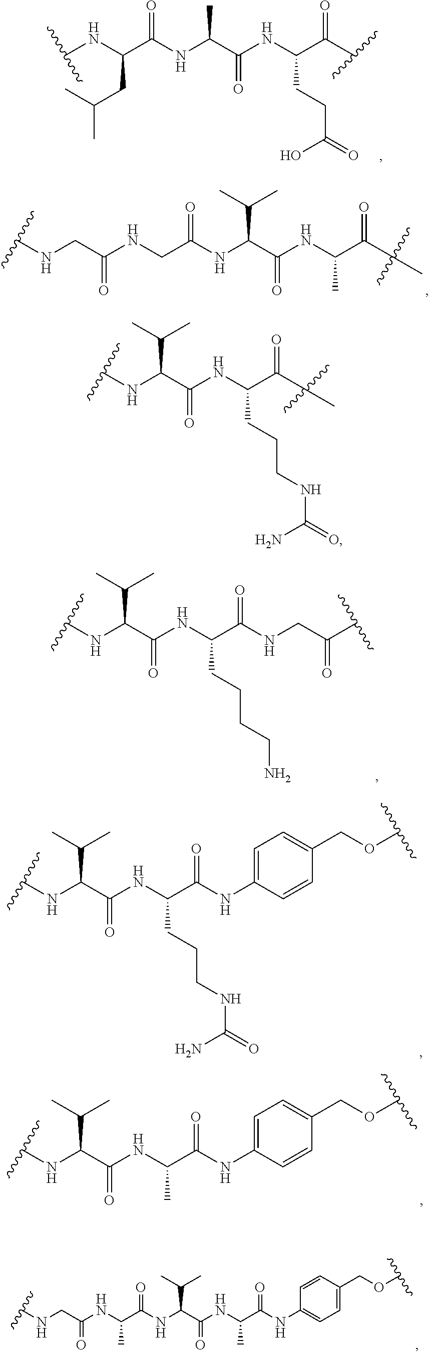 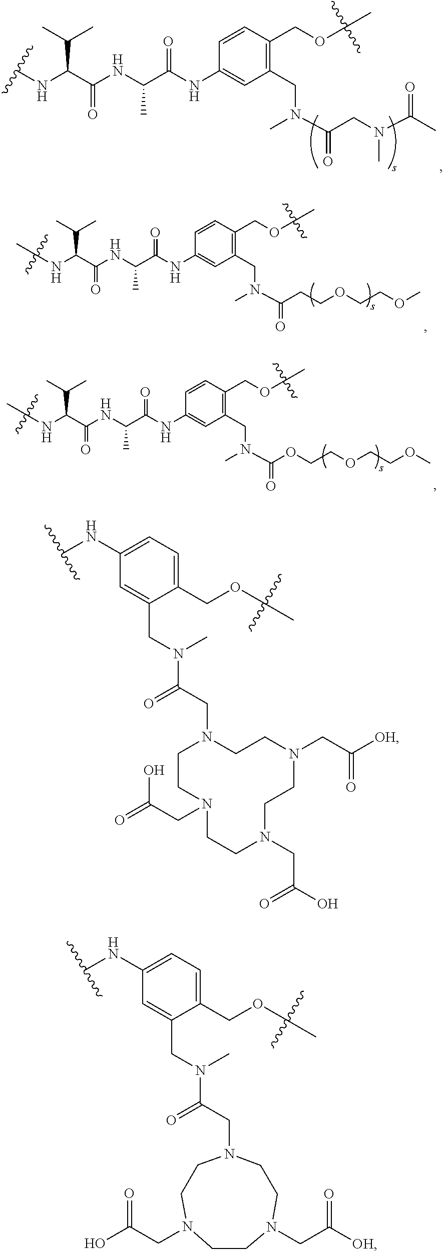 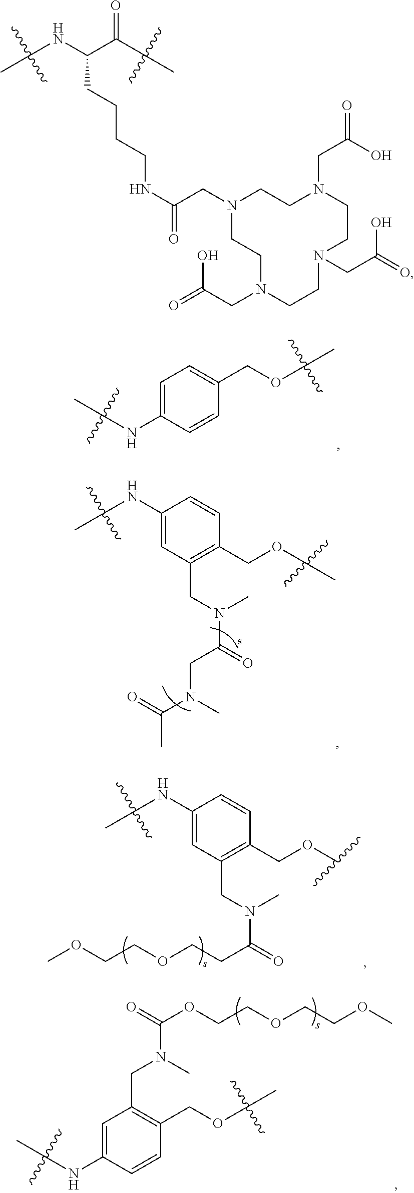 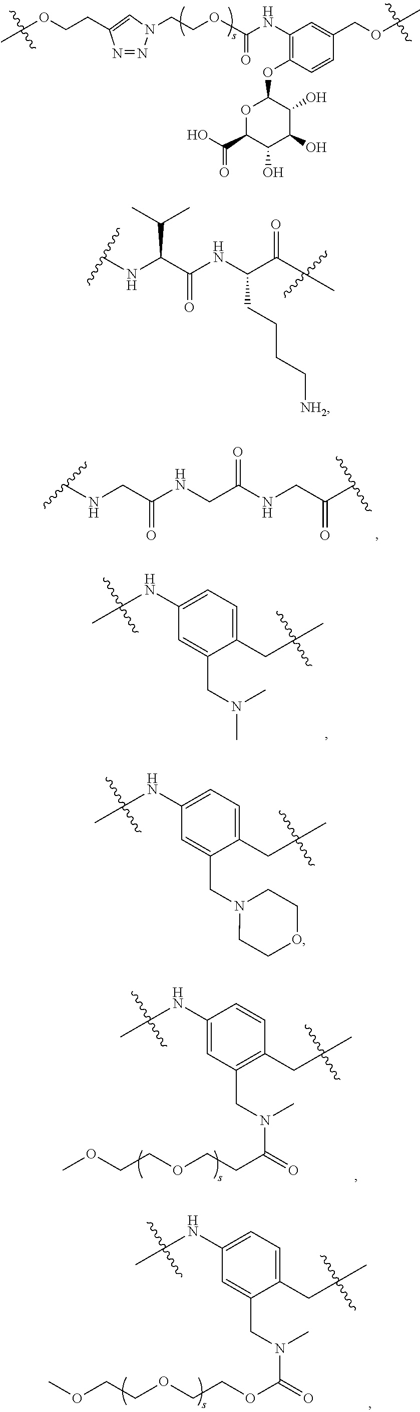 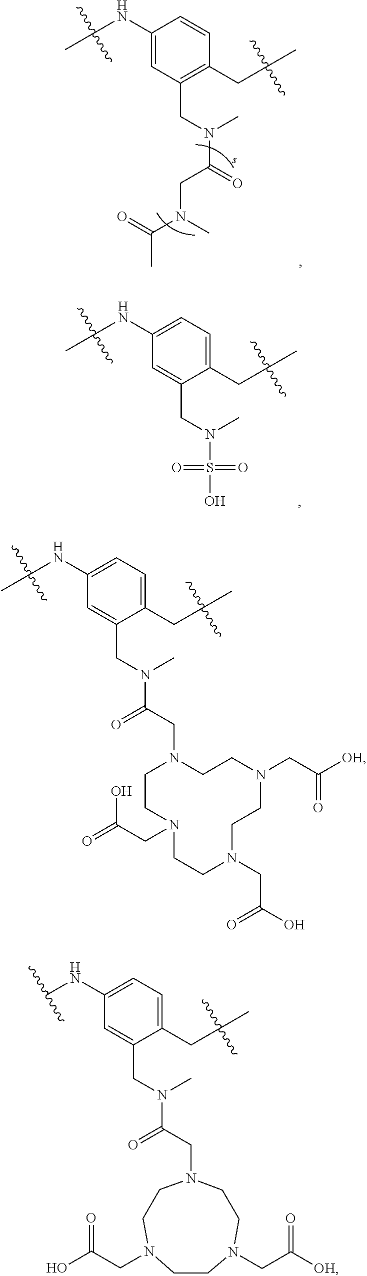 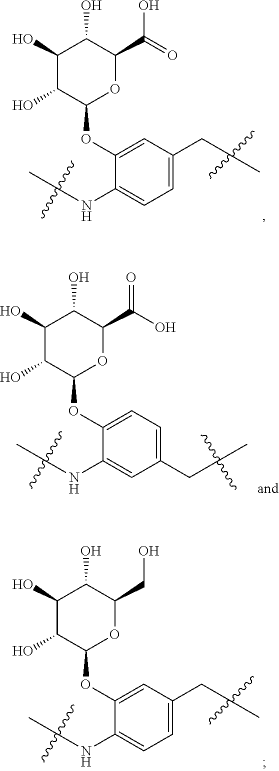
wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15;
preferably, L is selected from the group consisting of the substituted or unsubstituted structural fragments composed of one or more of the following groups: 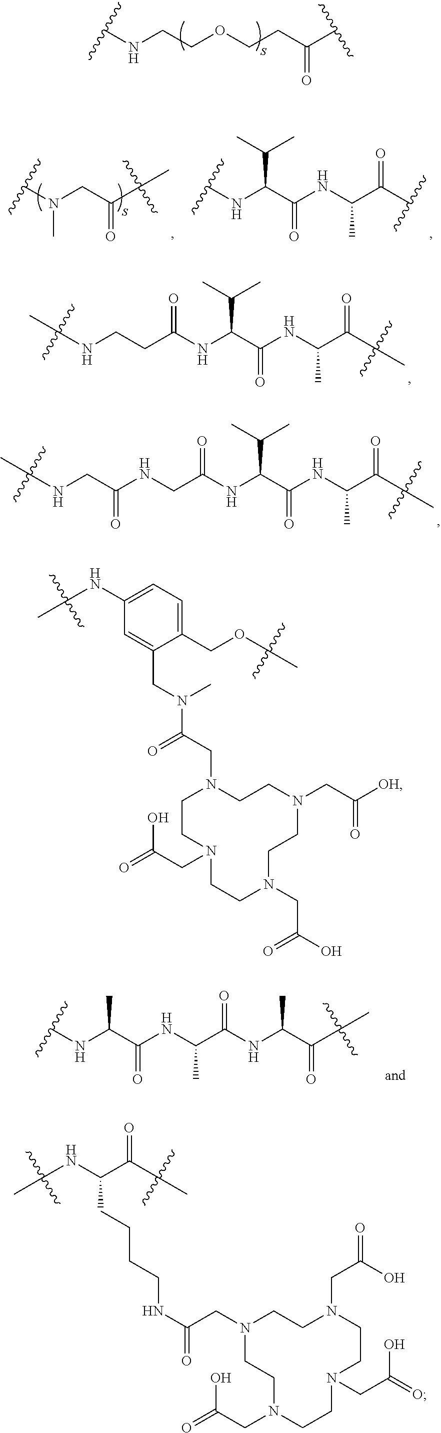
wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably, s is selected from 5, 8, 10;
preferably, L is selected from the group consisting of the substituted or unsubstituted structural fragments composed of one or more of the following groups: 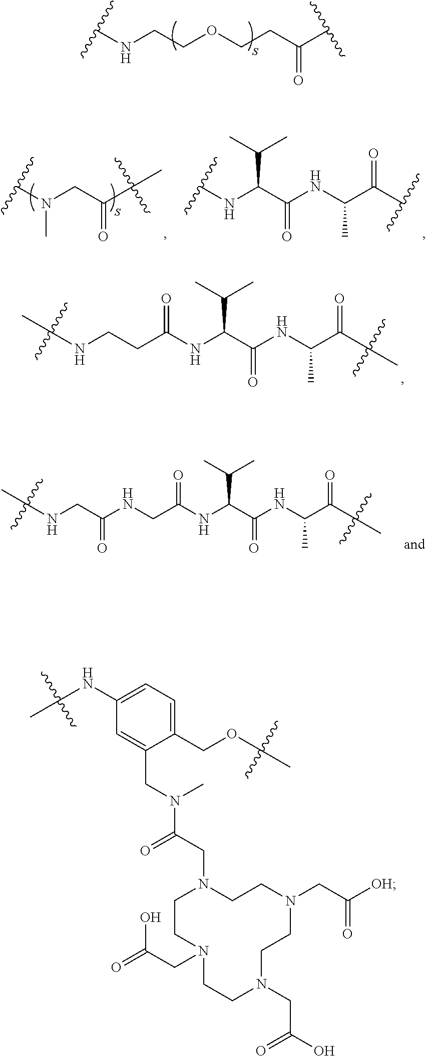
wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably, s is selected from 5, 8, 10;
preferably, L is selected from the group consisting of the substituted or unsubstituted structural fragments composed of one or more of the following groups: 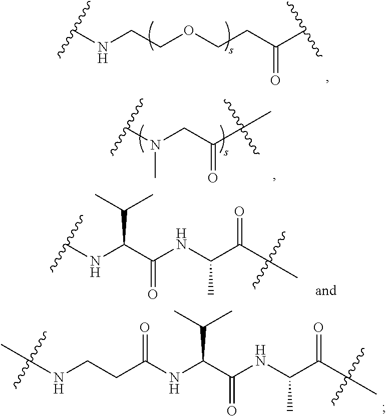
wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably, s is selected from 5, 8, 10;
preferably, L comprises the following structural fragment 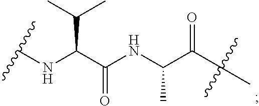 [SMILES: *N[C@H](C(=O)N[C@@H](C)C(*)=O)C(C)C]
preferably, L is formed by connecting a structural fragment of Group I with one or more substituted or unsubstituted structural fragments selected from Group II:
Group I is 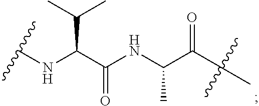 [SMILES: *N[C@H](C(=O)N[C@@H](C)C(*)=O)C(C)C]
Group II is composed of 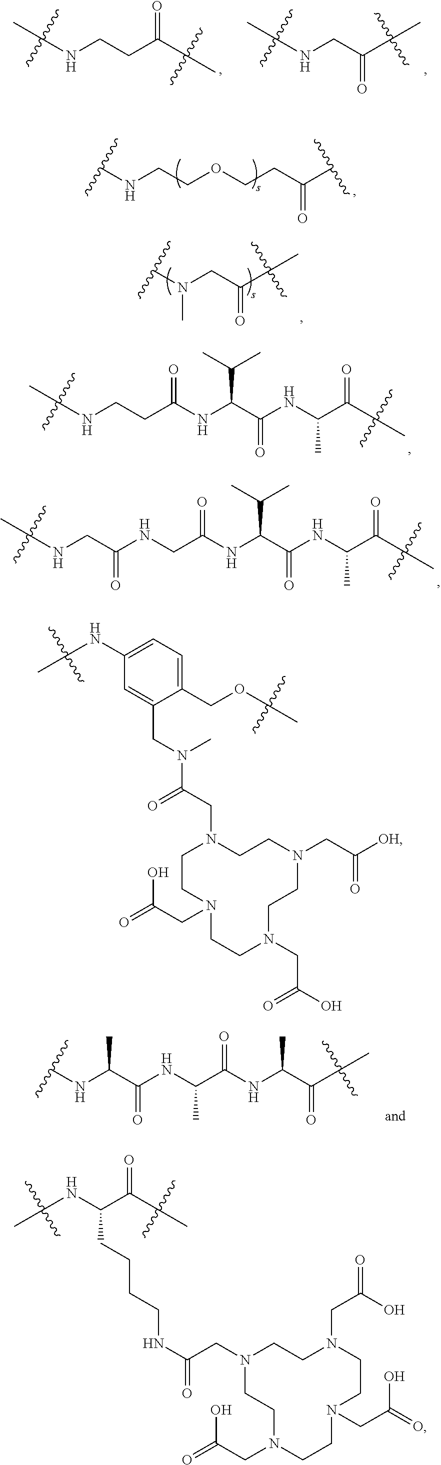
preferably Group II is composed of 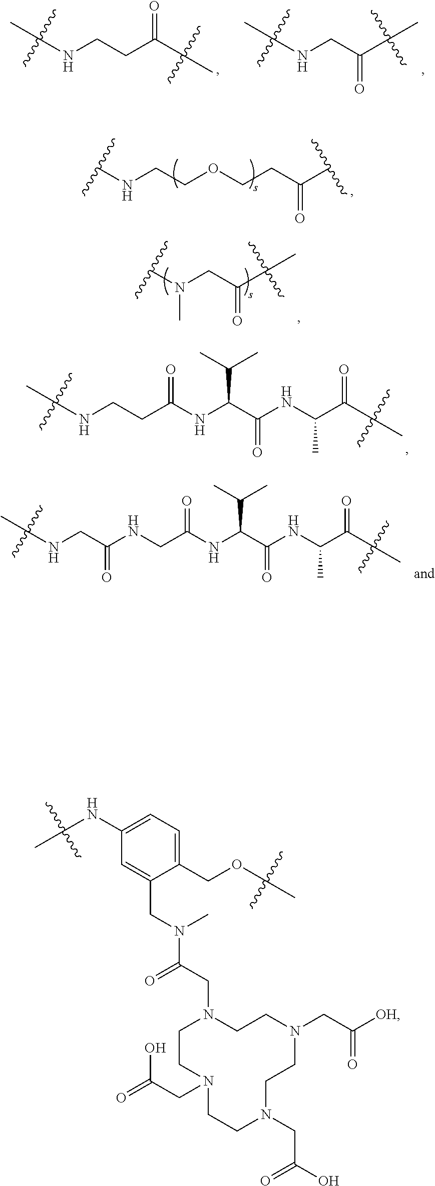
preferably Group II is composed of 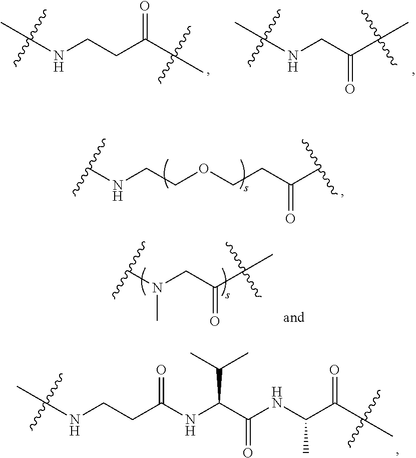
wherein s is an integer selected from 1 to 20, preferably s is an integer selected from 1 to 15, such as an integer of 1 to 12, 3 to 12, for example, s is 1, 2, 3, 4, 5, 6, 7, 8, 9, 10, 11, 12, 13, 14 or 15, preferably s is selected from 5, 8, 10;
or, L is selected from the group consisting of the substituted or unsubstituted structural fragments composed of one or more of the following groups: C₁₋₆ alkylene, 6- to 10-membered aryl, 5- to 6-membered heteroaryl, 9- to 12-membered nitrogen-containing heterocyclic, —N(R′)—, —NH (R′), —N(R′)₂, carbonyl group, —O—, natural amino acids or unnatural amino acids or analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly), 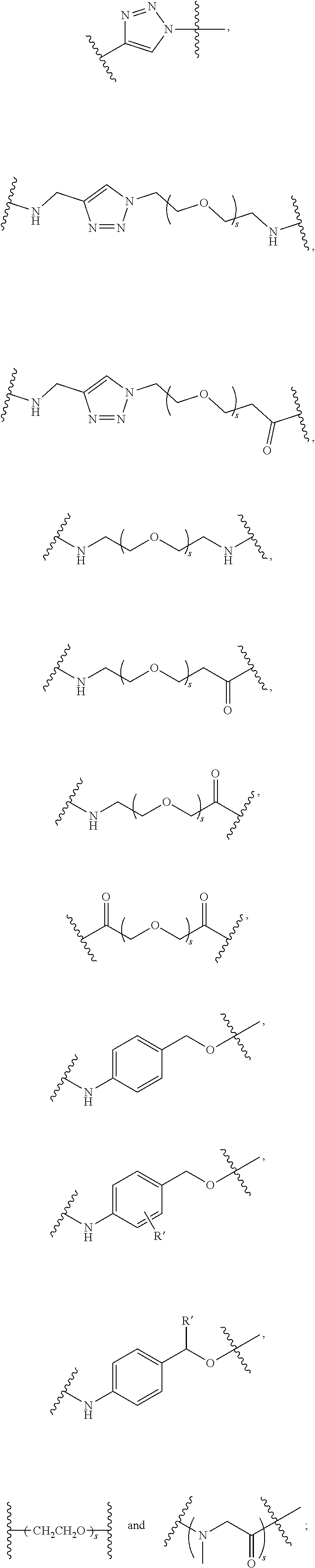
wherein R′ represents hydrogen, C₁₋₆ alkyl, —C₁₋₆ alkylene-CO₂H, —C₁₋₆ alkylene-SO₃H, —SO₃H, —PO₃H₂, —C₁₋₆ alkylene-NHC₁₋₆ alkyl, —C₁₋₆ alkylene-N(C₁₋₆ alkyl)₂, —CH₂N(C₁₋₆ alkyl)-C(═O)C₁₋₆ alkylene-heterocycle, —CH₂NH—SO₃H, —CH₂N(C₁₋₆ alkyl)-SO₃H, —CH₂NHC₁₋₆ alkylene-SO₃H, —CH₂N(C₁₋₆ alkyl)C₁₋₆ alkylene-SO₃H, —CH₂N(C₁₋₆ alkylene-SO₃H)₂, —CH₂N⁺(C₁₋₆ alkylene-SO₃H)₃, —CH₂N⁺(C₁₋₆ alkyl)₂-C₁₋₆ alkylene-SO₃H, —CH₂N(C₁₋₆ alkyl)-C(═O)C₁₋₆ alkylene-N⁺(C₁₋₆ alkylene-SO₃H)₃, —CH₂NH—C(—O)C₁₋₆ alkylene-N⁺(C₁₋₆ alkylene-SO₃H)₃, —CH₂N(C₁₋₆ alkyl)-C(═O)C₁₋₆ alkylene-N⁺(C₁₋₆ alkyl)₃, —CH₂NH—C(═O)C₁₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₃, —CH₂N(C₁₋₆ alkyl)-C(═O)OC₂₋₆ alkylene-N⁺(C₁₋₆ alkyl)₂-CH₂CO₂H, —CH₂N(C₁₋₆ alkyl)-C₁₋₆ alkylene-CO₂H, —CH₂N⁺(C₁₋₆ alkyl)₂-C₁₋₆ alkylene-CO₂H, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—(OCH₂CH₂)ᵣ—OC₁₋₆ alkyl, —(CH₂N(Me)-C(═O))ᵣ—C₁₋₆ alkyl, or polyethylene glycol fragment containing 1 to 10 EO units (i.e. —(CH₂CH₂O)ᵣ—C₁₋₆ alkyl), wherein r is an integer selected from 1 to 20; s is an integer selected from 1 to 20;
preferably, L is selected from the group consisting of the substituted or unsubstituted structural fragments composed of one or more of the following groups: 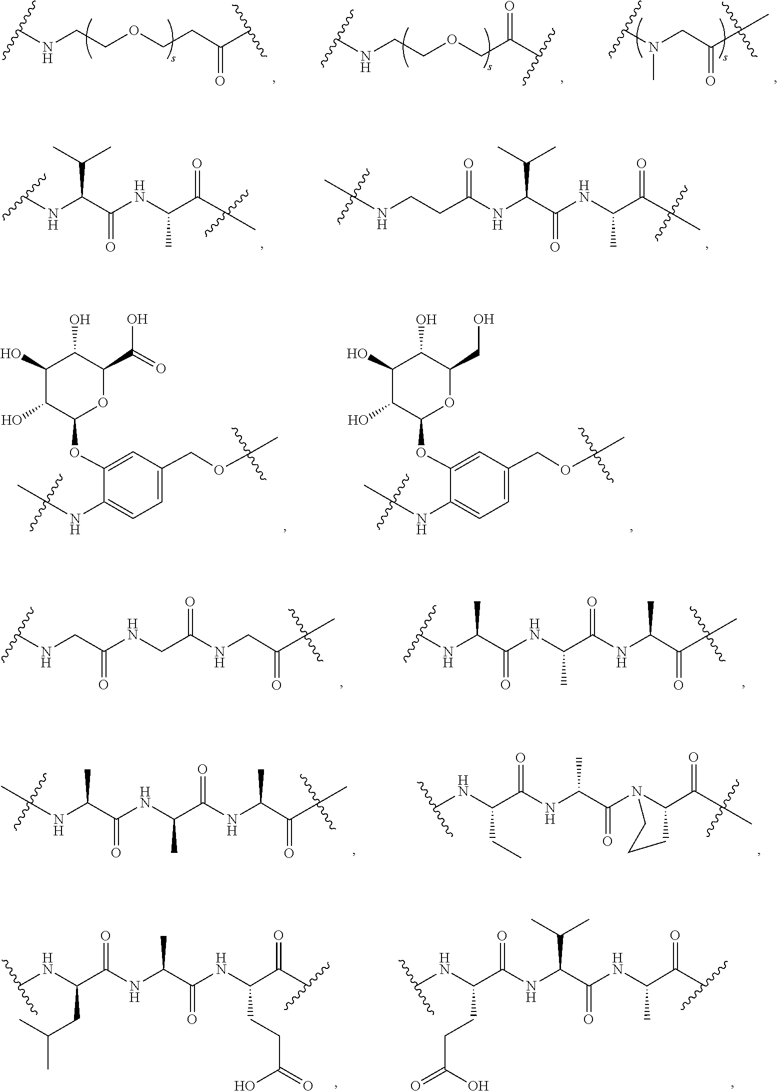 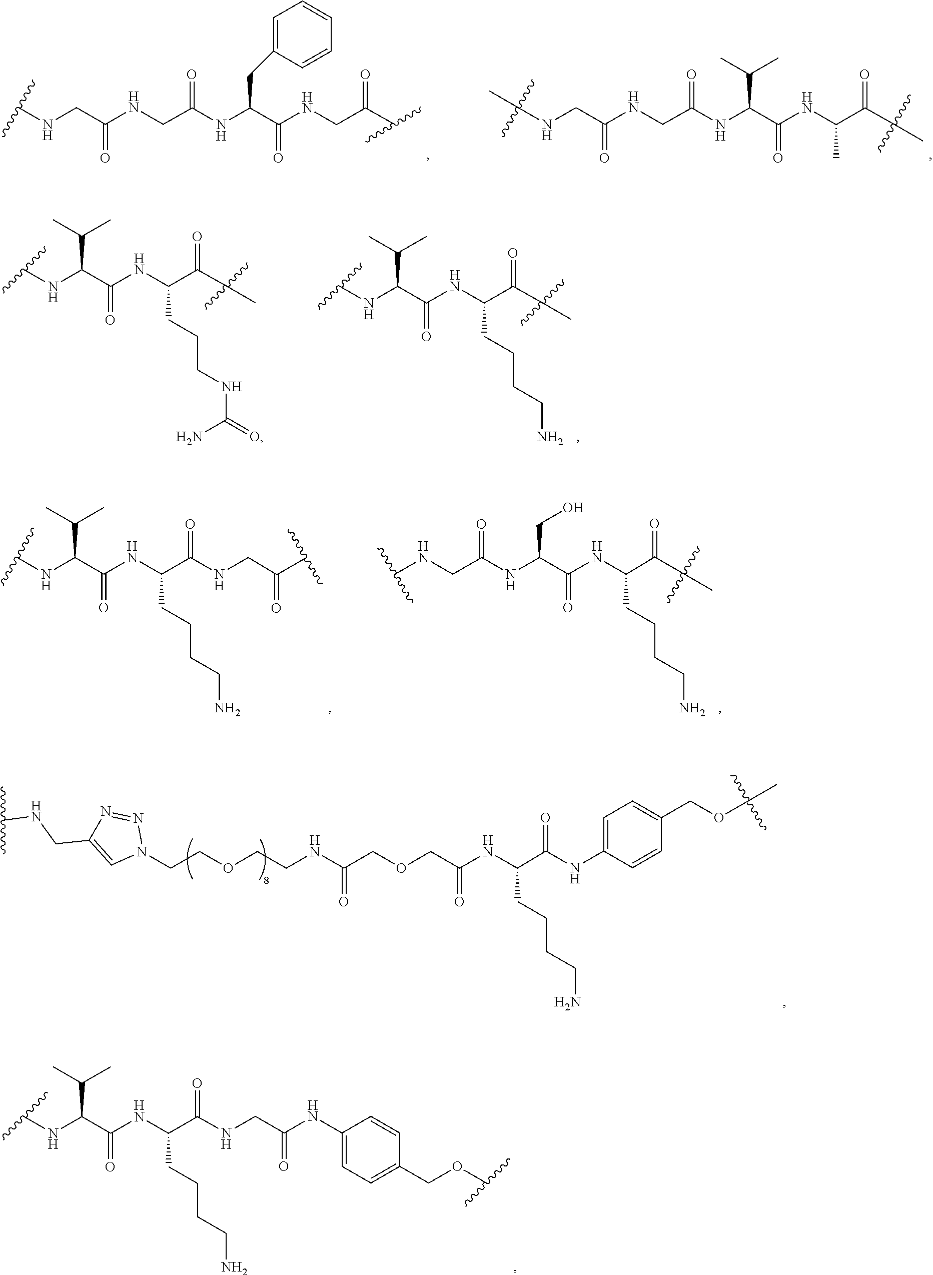 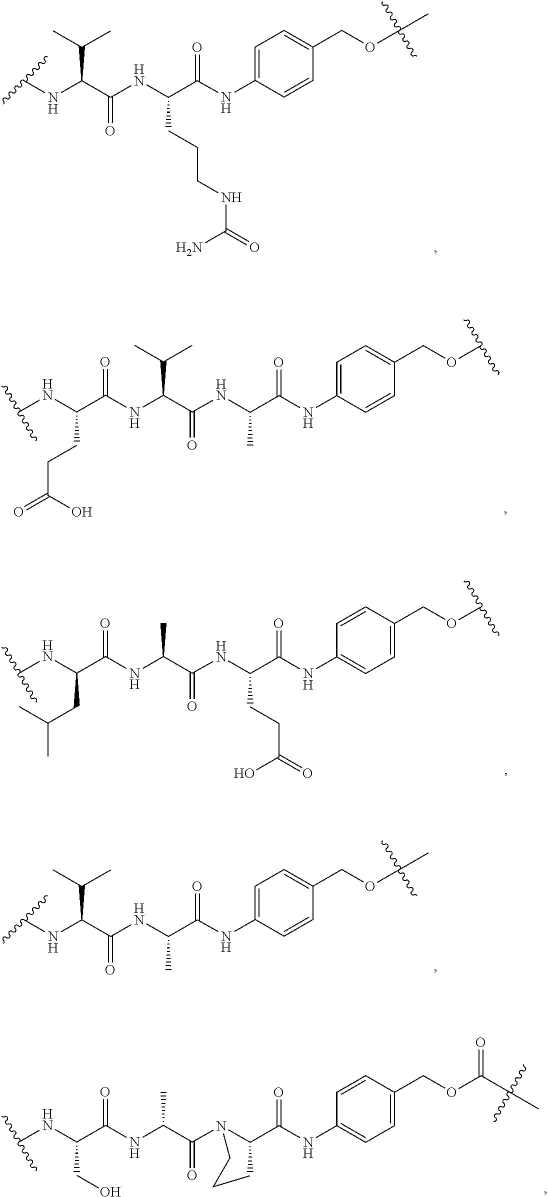 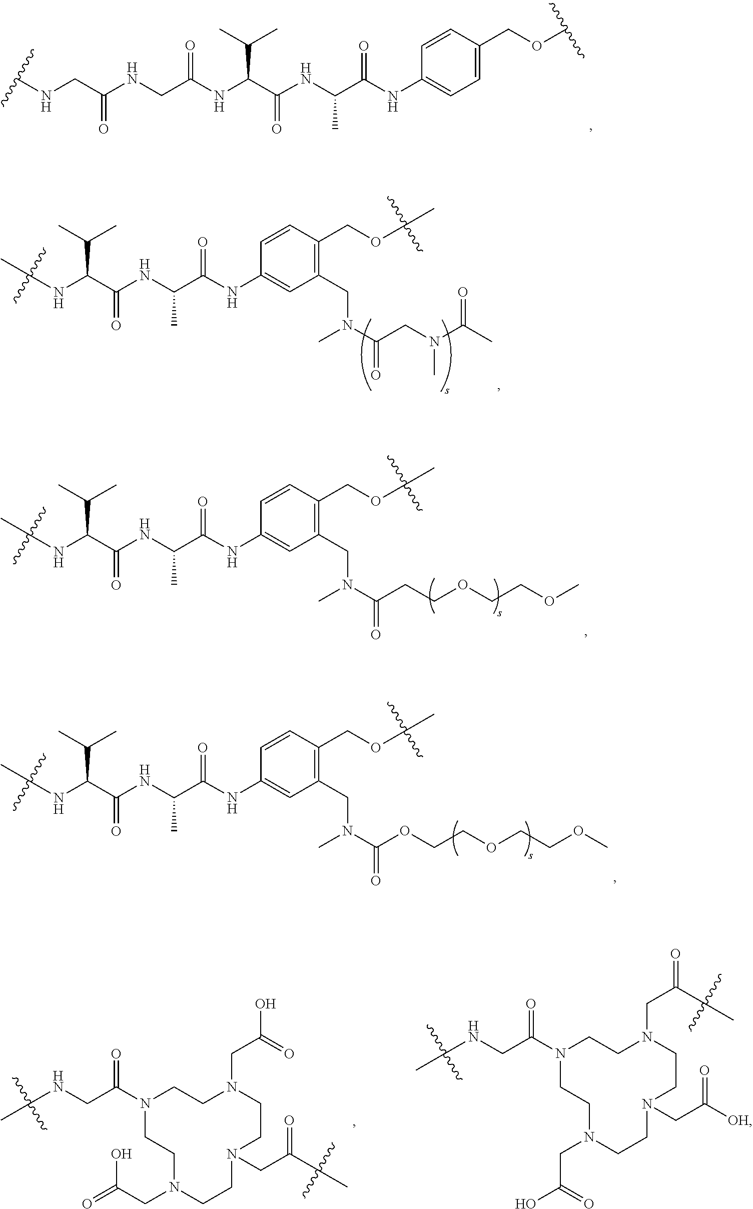 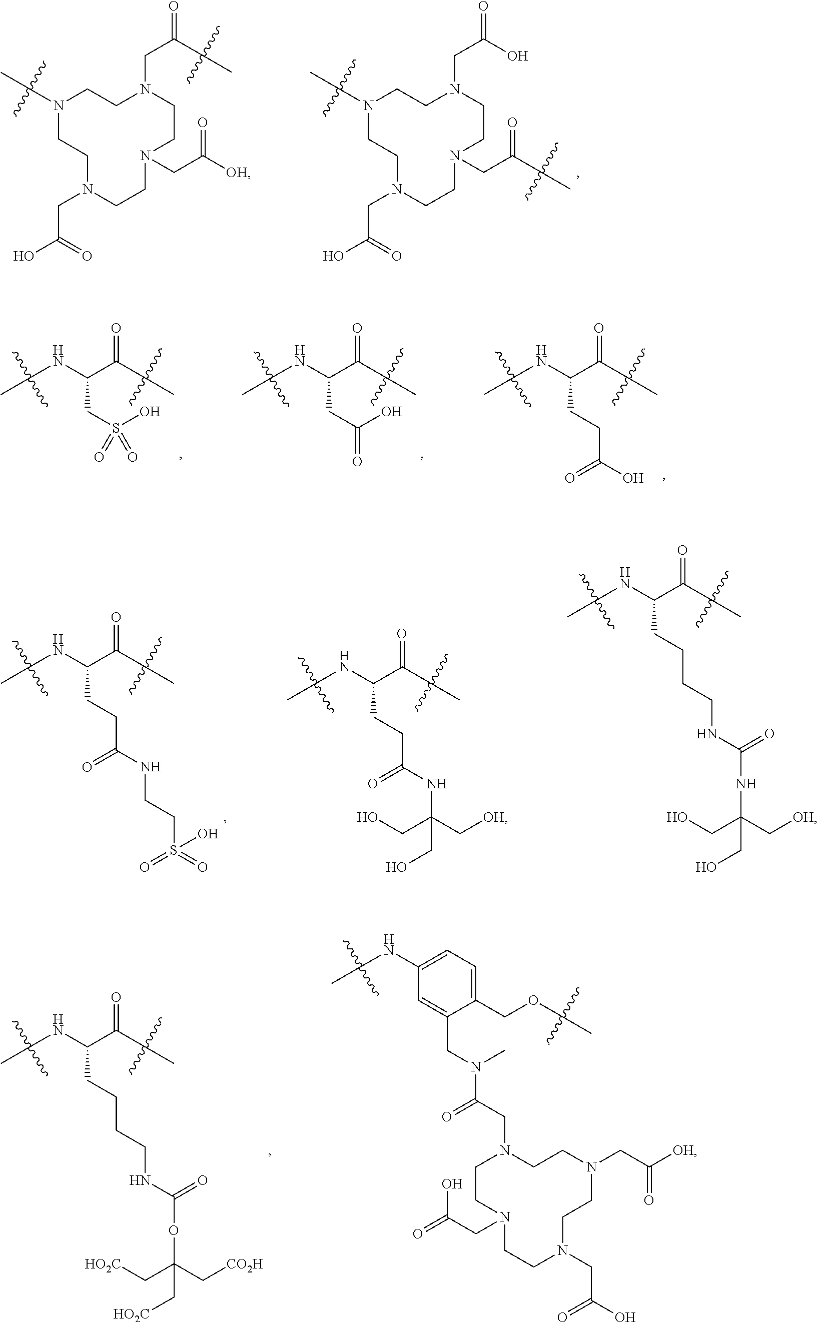 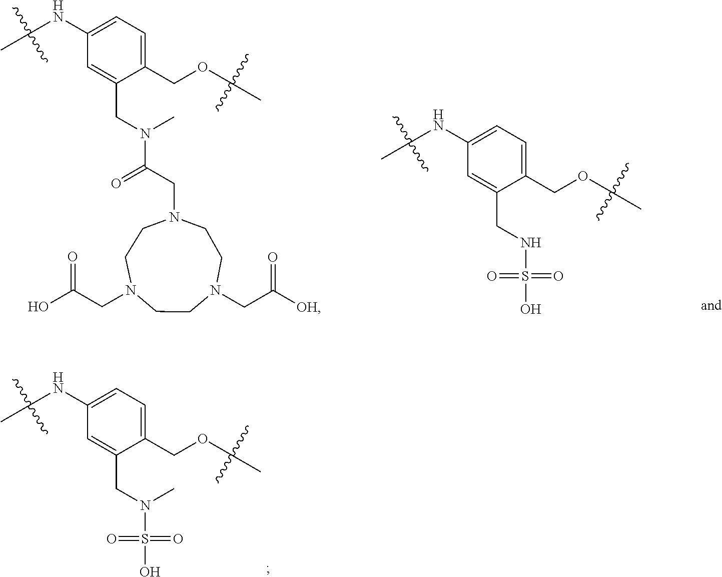 [SMILES: *Nc1ccc(CO*)c(CN(C)C(=O)CN2CCN(CC(=O)O)CCN(CC(=O)O)CC2)c1 ; *Nc1ccc(CO*)c(CNS(=O)(=O)O)c1 ; *Nc1ccc(CO*)c(CN(C)S(=O)(=O)O)c1]
s is an integer selected from 1 to 20;
or, L is selected from the group consisting of divalent substituted or unsubstituted structural fragments composed of one or more of the following groups: C₁₋₆ alkylene, 6- to 10-membered aryl, 5- to 6-membered heteroaryl, —N(R′)—, carbonyl group, —O—, natural amino acids or unnatural amino acids or analogs thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), short peptides composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly), 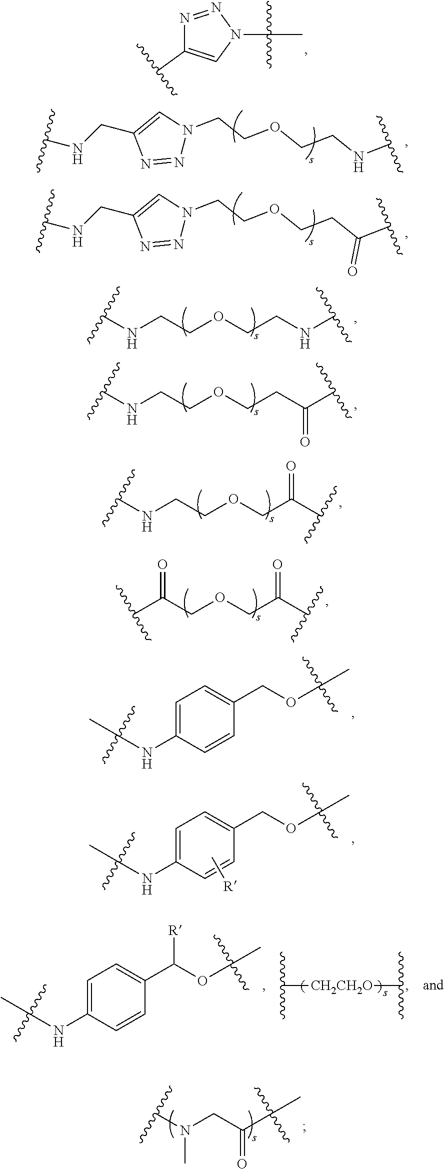
wherein R′ represents hydrogen, C₁₋₆ alkyl, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, or polyethylene glycol fragment containing 1 to 10 EO units (i.e. —(CH₂CH₂O)ᵣ—C₁₋₆ alkyl), wherein r is an integer selected from 1 to 20; s is an integer selected from 1 to 20;
preferably, L is selected from the group consisting of divalent substituted or unsubstituted structural fragments composed of one or more of the following groups: 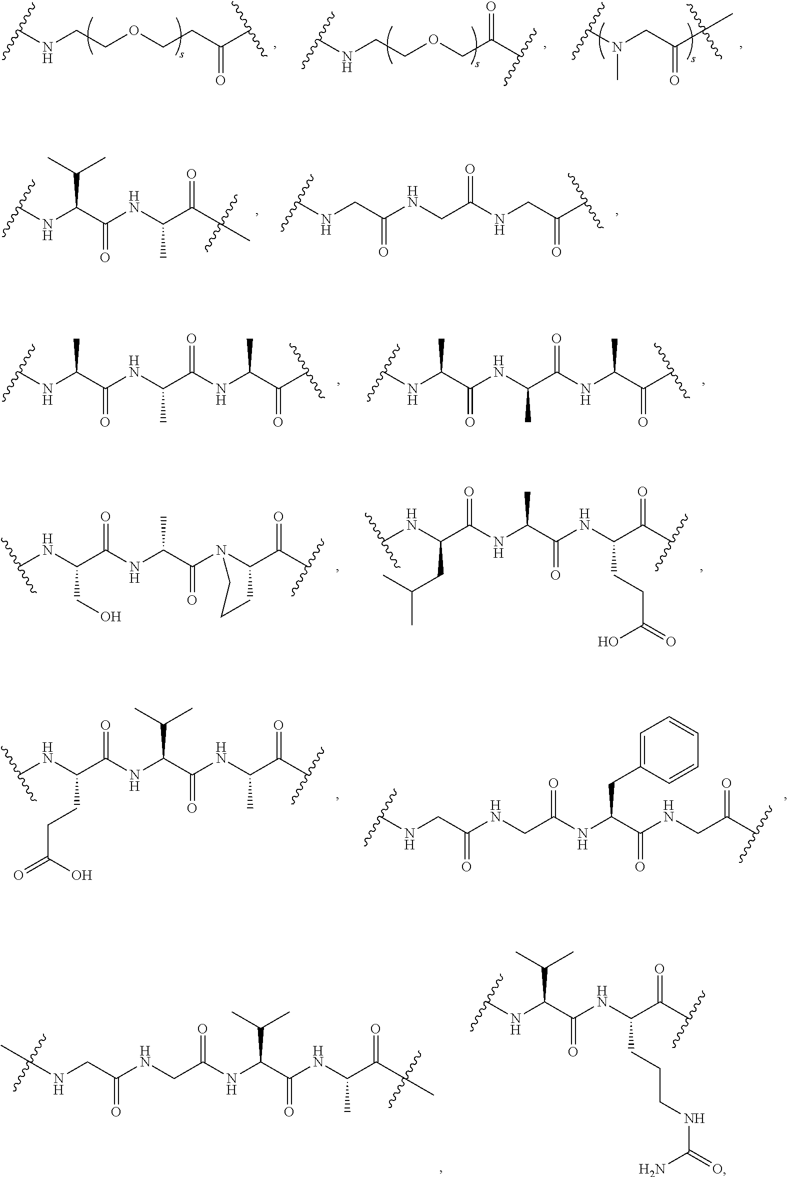 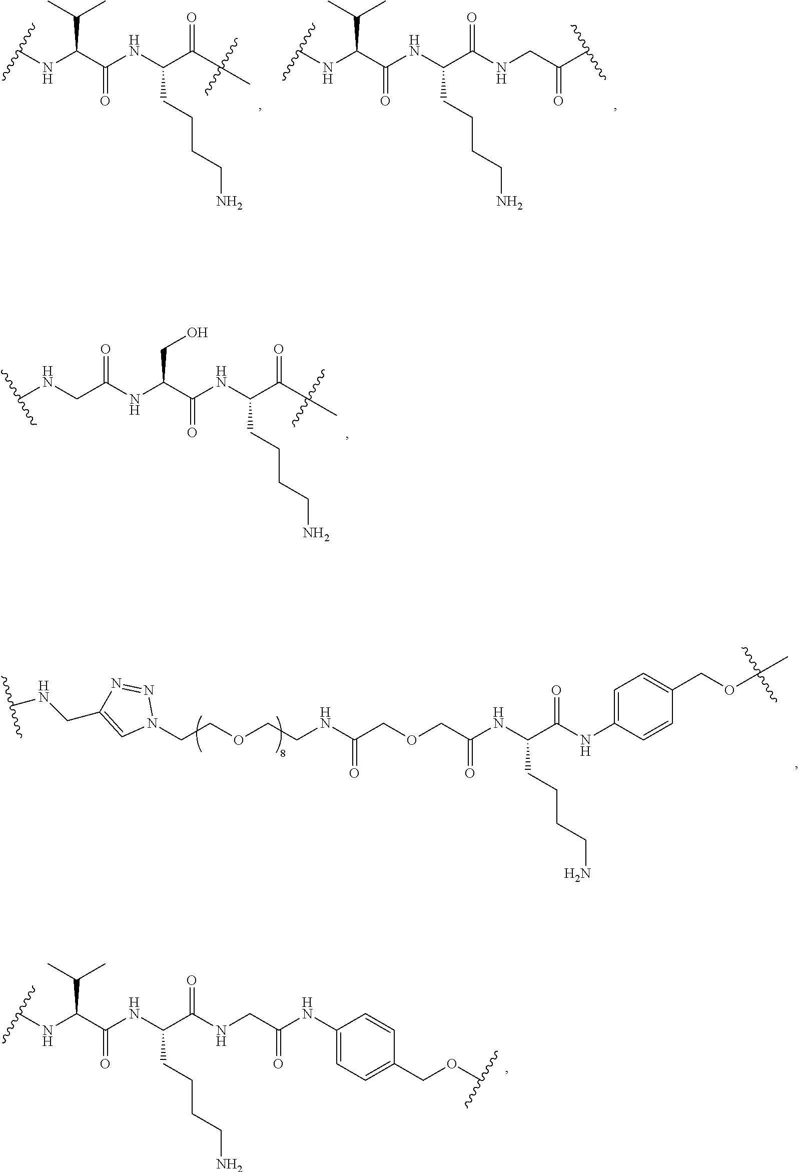 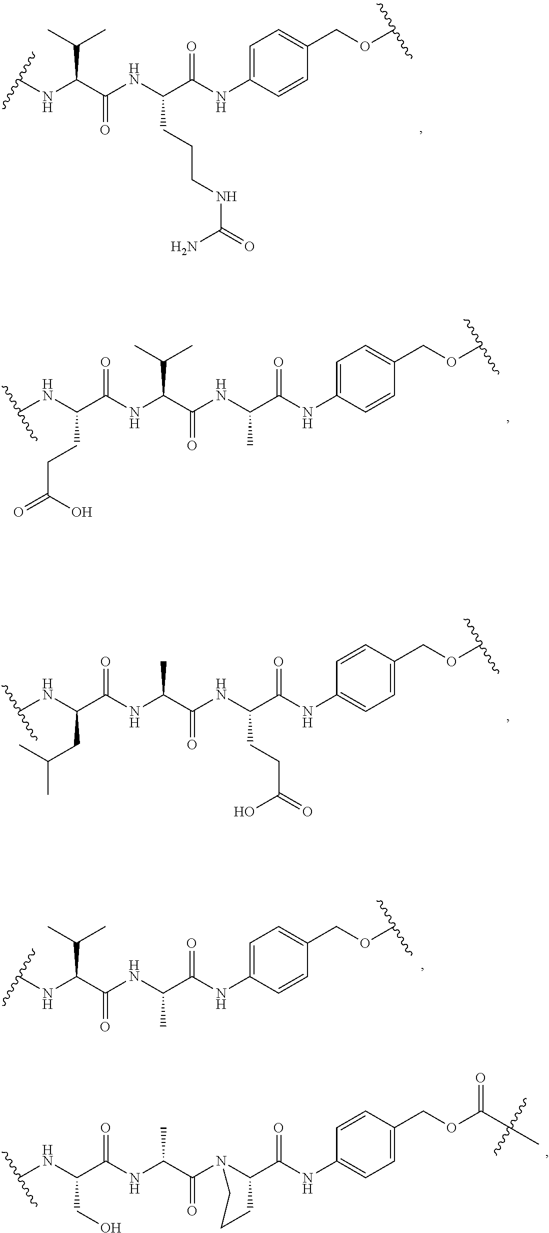 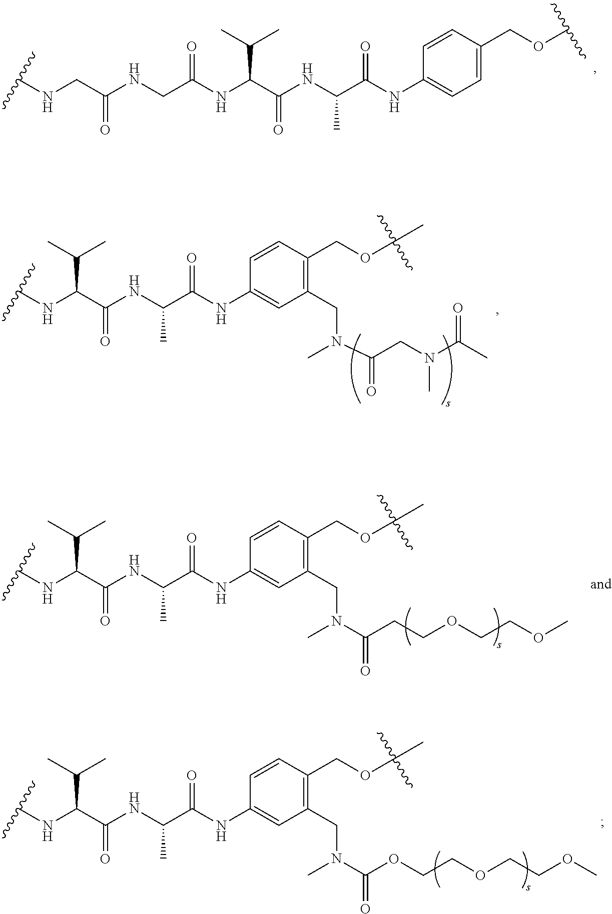
s is an integer selected from 1 to 20.

5. The antibody-drug conjugate according to claim 1, wherein E is a single bond, substituted or unsubstituted —NH—CH₂—, or is selected from the group consisting of the following substituted or unsubstituted structural fragments: 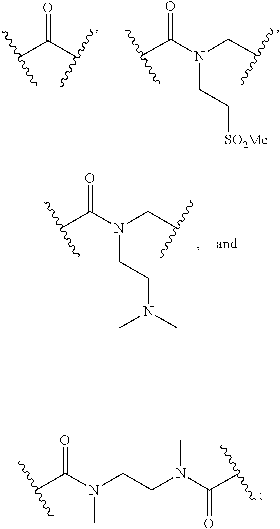
preferably, E is a single bond, substituted or unsubstituted-NH—CH₂—, or

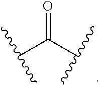 [SMILES: *C(*)=O]

6. The antibody-drug conjugate according to claim 1, wherein the cytotoxic drug is selected from the group consisting of anti-tubulin agents, DNA intercalators, DNA topoisomerase inhibitors, RNA polymerase inhibitors and gene transcription inhibitors;
preferably, the cytotoxic drug is a topoisomerase I inhibitor (e.g., camptothecin, hydroxycamptothecin, 9-aminocamptothecin, SN-38, irinotecan, exatecan, topotecan, belotecan, rubitecan, diflomotecan, lurtotecan, karenitecin, gimatecan, namitecan, simmitecan, chimmitecan, silatecan, or elomotecan);
preferably, the cytotoxic drug is selected from the group consisting of the following compounds or isotopically labeled compounds thereof: 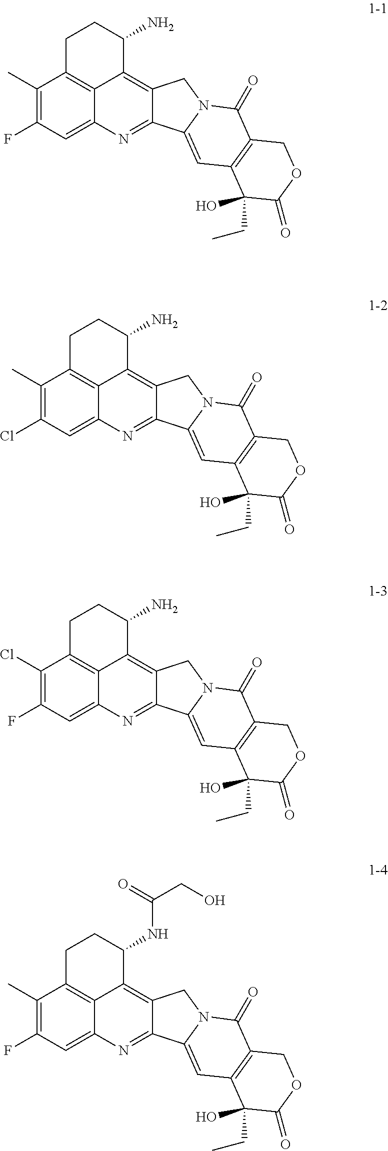 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3] 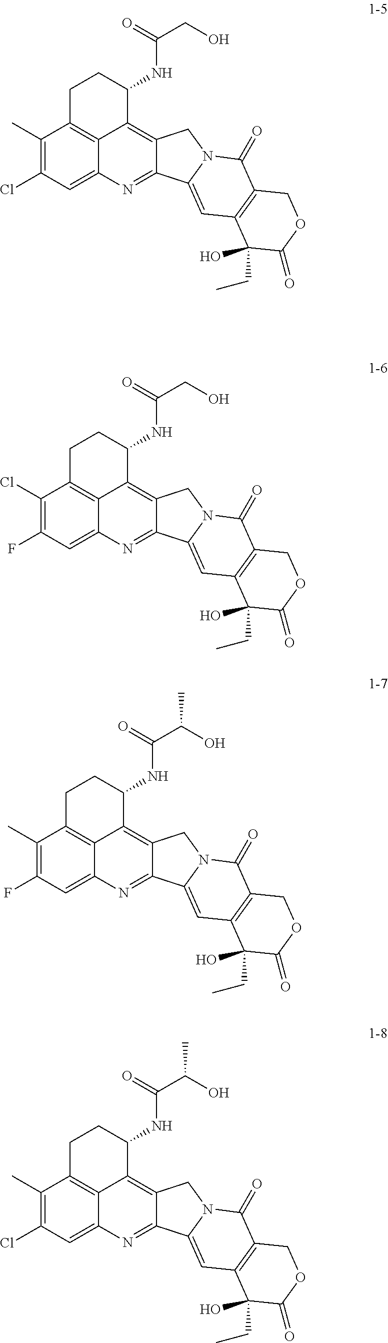 [SMILES: CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3] 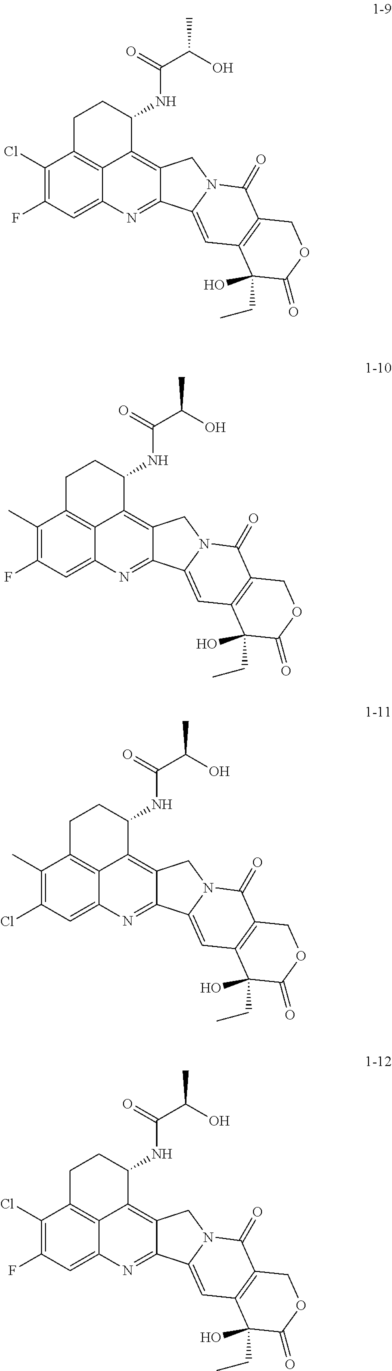 [SMILES: CC[C@@]1(O)C(=O)OCc2c1cc1n(c2=O)Cc2c-1nc1cc(F)c(Cl)c3c1c2[C@@H](NC(=O)[C@H](C)O)CC3 ; CC[C@@]1(O)C(=O)OCc2c1cc1n(c2=O)Cc2c-1nc1cc(F)c(C)c3c1c2[C@@H](NC(=O)[C@@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3 ; CC[C@@]1(O)C(=O)OCc2c1cc1n(c2=O)Cc2c-1nc1cc(F)c(Cl)c3c1c2[C@@H](NC(=O)[C@@H](C)O)CC3] 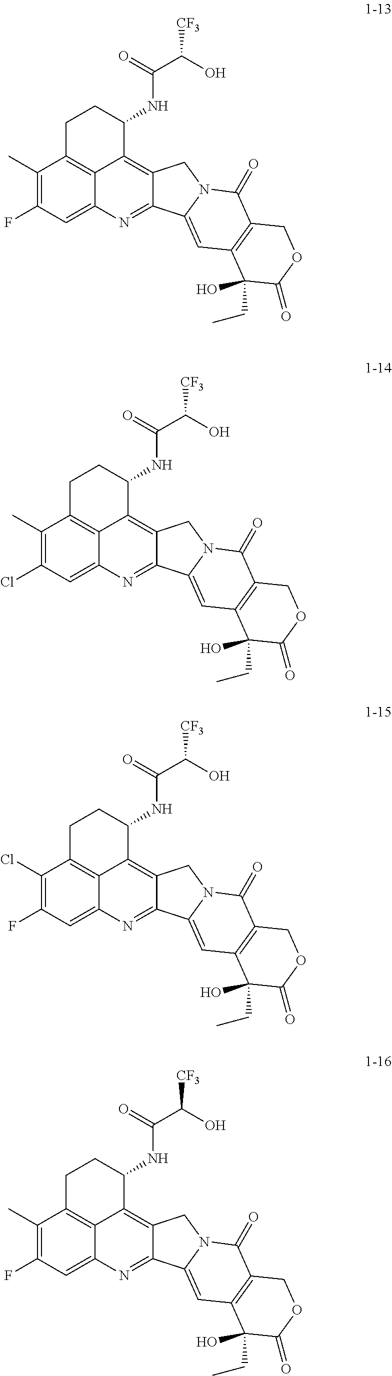 [SMILES: CC[C@@]1(O)C(=O)OCc2c1cc1n(c2=O)Cc2c-1nc1cc(F)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(Cl)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(C)c3c1c2[C@@H](NC(=O)[C@H](O)C(F)(F)F)CC3] 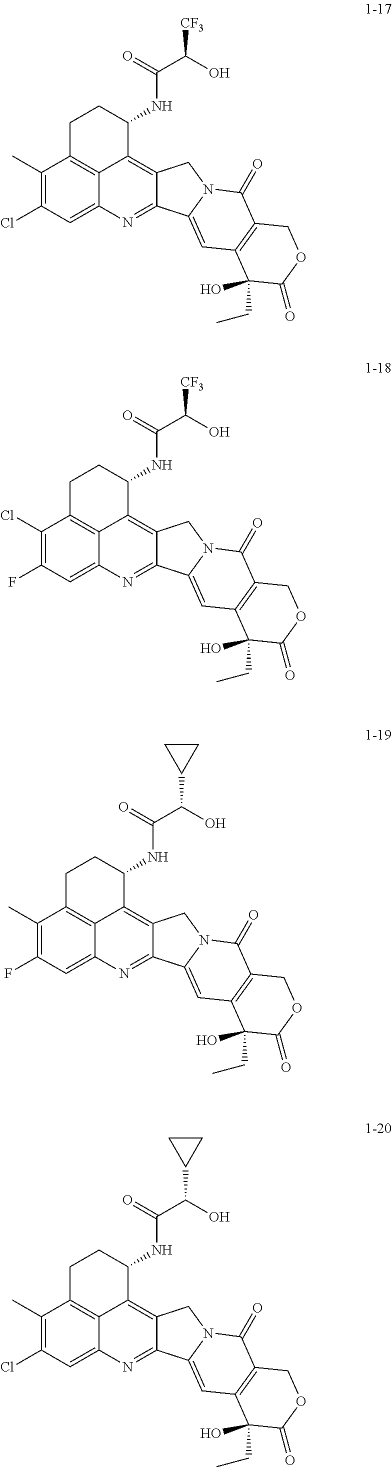 [SMILES: CC[C@@]1(O)C(=O)OCc2c1cc1n(c2=O)Cc2c-1nc1cc(Cl)c(C)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(C)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@@H](O)C1CC1)CC3] 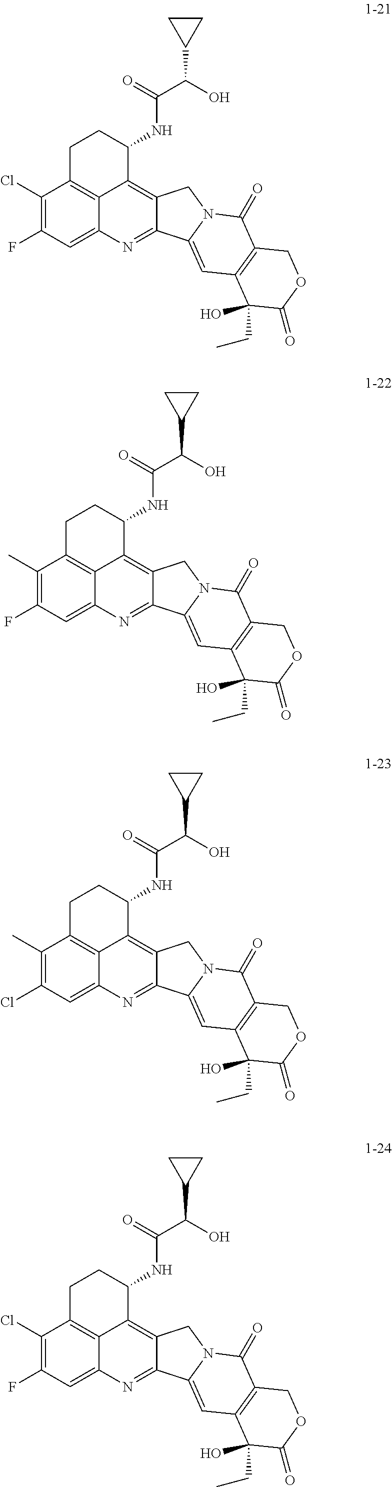 [SMILES: CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(F)c(C)c3c1c2[C@@H](NC(=O)[C@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3 ; CC[C@@]1(O)C(=O)OCc2c1cc1n(c2=O)Cc2c-1nc1cc(F)c(Cl)c3c1c2[C@@H](NC(=O)[C@H](O)C1CC1)CC3] 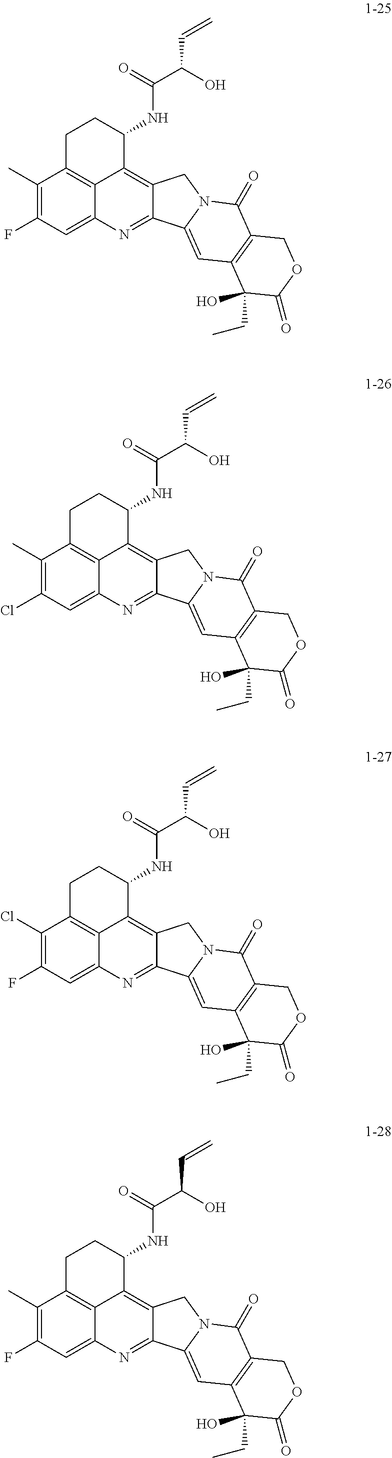 [SMILES: C=C[C@H](O)C(=O)N[C@H]1CCc2c(C)c(F)cc3nc4c(c1c23)Cn1c-4cc2c(c1=O)COC(=O)[C@]2(O)CC ; C=C[C@H](O)C(=O)N[C@H]1CCc2c(C)c(Cl)cc3nc4c(c1c23)Cn1c-4cc2c(c1=O)COC(=O)[C@]2(O)CC ; C=C[C@H](O)C(=O)N[C@H]1CCc2c(Cl)c(F)cc3nc4c(c1c23)Cn1c-4cc2c(c1=O)COC(=O)[C@]2(O)CC ; C=C[C@@H](O)C(=O)N[C@H]1CCc2c(C)c(F)cc3nc4c(c1c23)Cn1c-4cc2c(c1=O)COC(=O)[C@]2(O)CC] 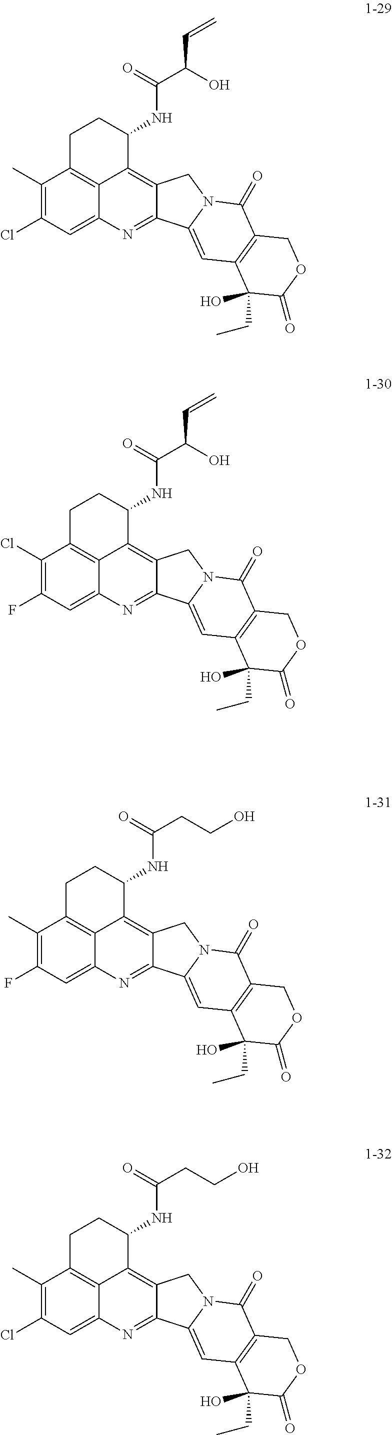 [SMILES: C=C[C@@H](O)C(=O)N[C@H]1CCc2c(C)c(Cl)cc3nc4c(c1c23)Cn1c-4cc2c(c1=O)COC(=O)[C@]2(O)CC ; C=C[C@@H](O)C(=O)N[C@H]1CCc2c(Cl)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2[C@@H](NC(=O)CCO)CC3 ; CC[C@@]1(O)C(=O)OCc2c1cc1n(c2=O)Cc2c-1nc1cc(Cl)c(C)c3c1c2[C@@H](NC(=O)CCO)CC3] 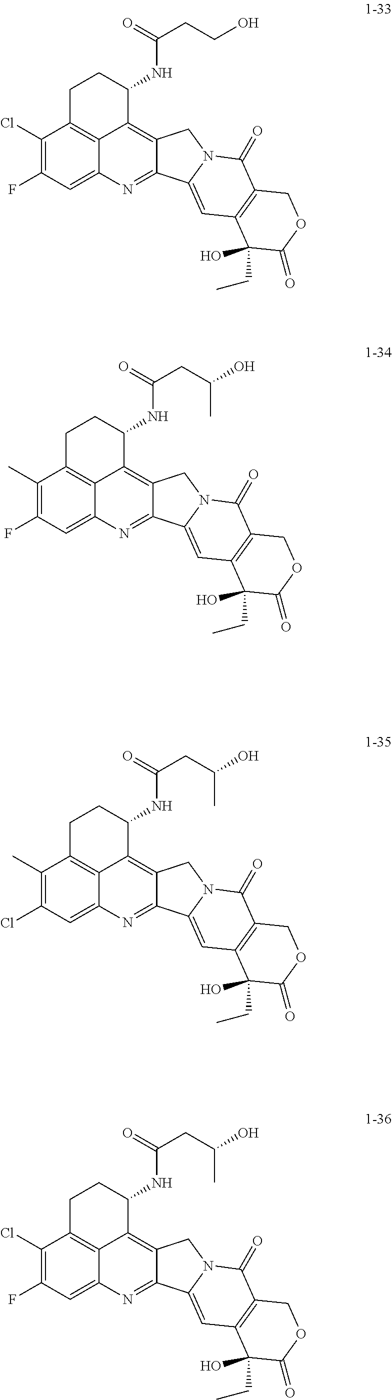 [SMILES: CC[C@@]1(O)C(=O)OCc2c1cc1n(c2=O)Cc2c-1nc1cc(F)c(Cl)c3c1c2[C@@H](NC(=O)CCO)CC3 ; CC[C@@]1(O)C(=O)OCc2c1cc1n(c2=O)Cc2c-1nc1cc(F)c(C)c3c1c2[C@@H](NC(=O)C[C@@H](C)O)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3] 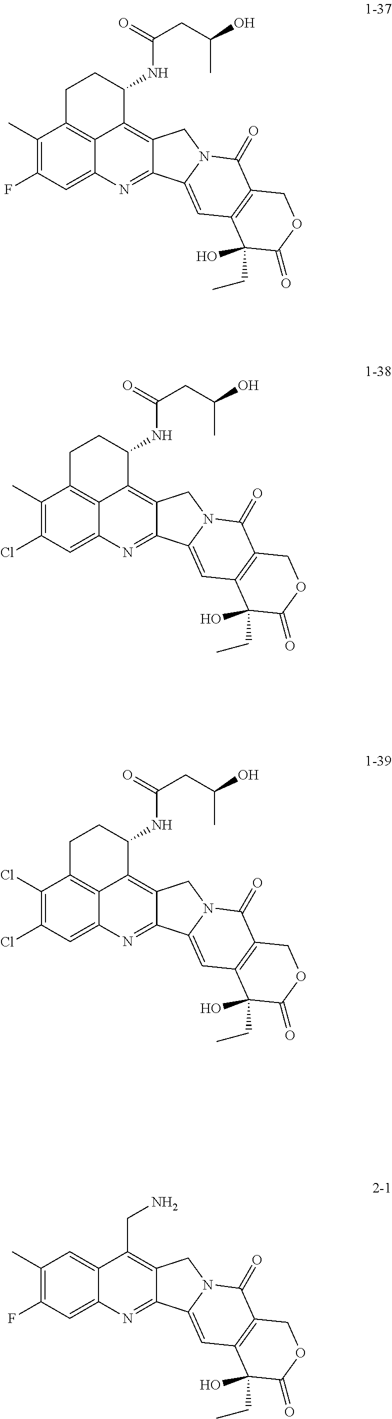 [SMILES: CC[C@@]1(O)C(=O)OCc2c1cc1n(c2=O)Cc2c-1nc1cc(F)c(C)c3c1c2[C@@H](NC(=O)C[C@H](C)O)CC3 ; CC[C@@]1(O)C(=O)OCc2c1cc1n(c2=O)Cc2c-1nc1cc(Cl)c(C)c3c1c2[C@@H](NC(=O)C[C@H](C)O)CC3 ; CC[C@@]1(O)C(=O)OCc2c1cc1n(c2=O)Cc2c-1nc1cc(Cl)c(Cl)c3c1c2[C@@H](NC(=O)C[C@H](C)O)CC3 ; CC[C@@]1(O)C(=O)OCc2c1cc1n(c2=O)Cc2c-1nc1cc(F)c(C)cc1c2CN] 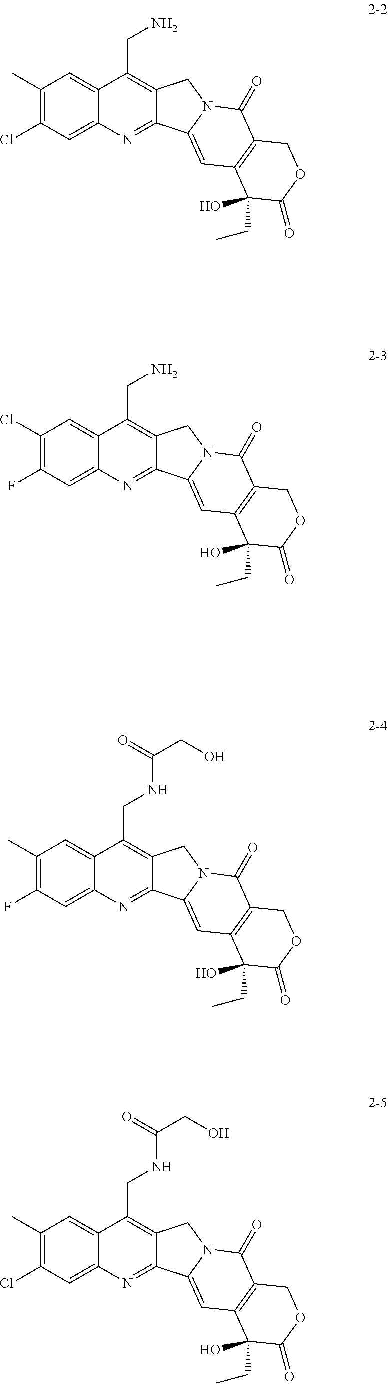 [SMILES: CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(F)c(C)cc1c2CNC(=O)CO ; CC[C@@]1(O)C(=O)OCc2c1cc1n(c2=O)Cc2c-1nc1cc(Cl)c(C)cc1c2CNC(=O)CO] 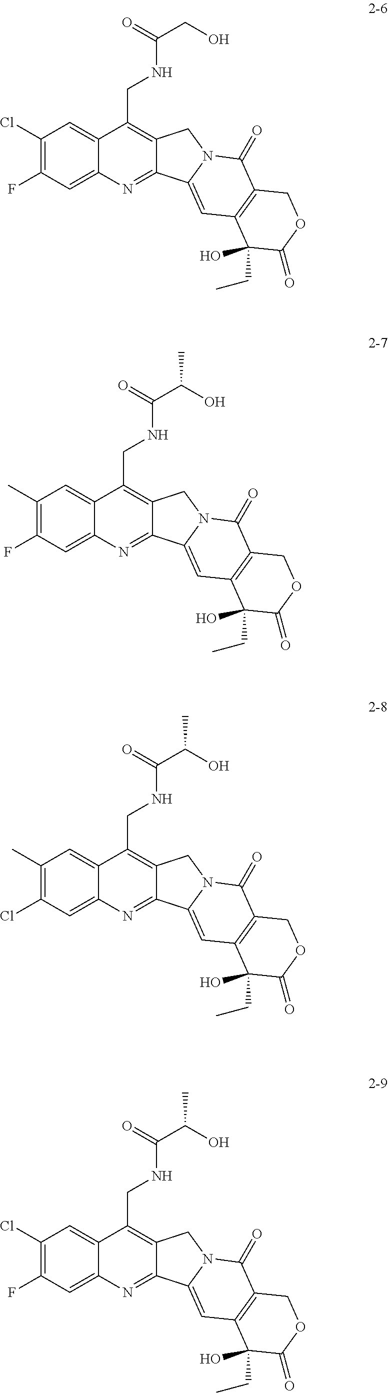 [SMILES: CC[C@@]1(O)C(=O)OCc2c1cc1n(c2=O)Cc2c-1nc1cc(F)c(Cl)cc1c2CNC(=O)CO ; CC[C@@]1(O)C(=O)OCc2c1cc1n(c2=O)Cc2c-1nc1cc(F)c(C)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O] 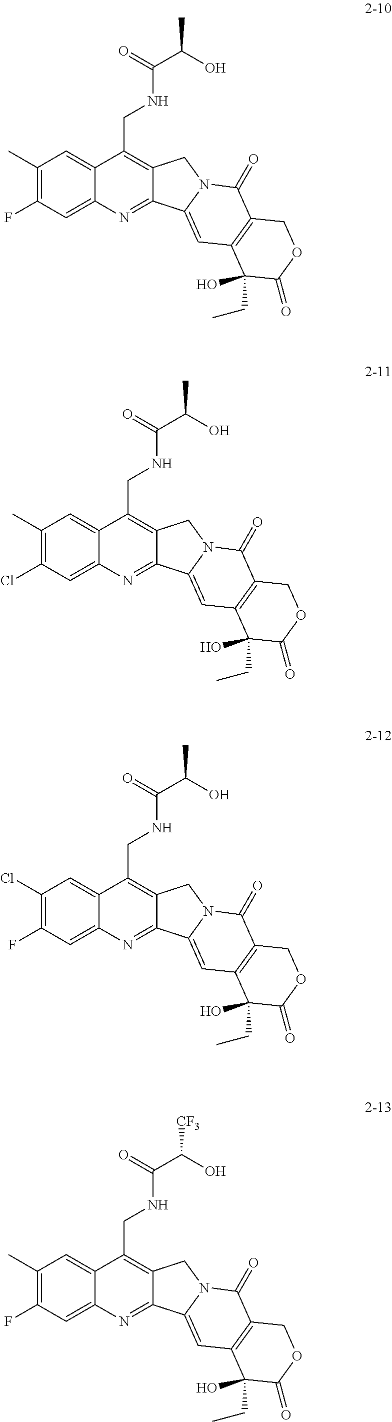 [SMILES: CC[C@@]1(O)C(=O)OCc2c1cc1n(c2=O)Cc2c-1nc1cc(F)c(C)cc1c2CNC(=O)[C@@H](C)O ; CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O ; CC[C@@]1(O)C(=O)OCc2c1cc1n(c2=O)Cc2c-1nc1cc(F)c(C)cc1c2CNC(=O)[C@@H](O)C(F)(F)F] 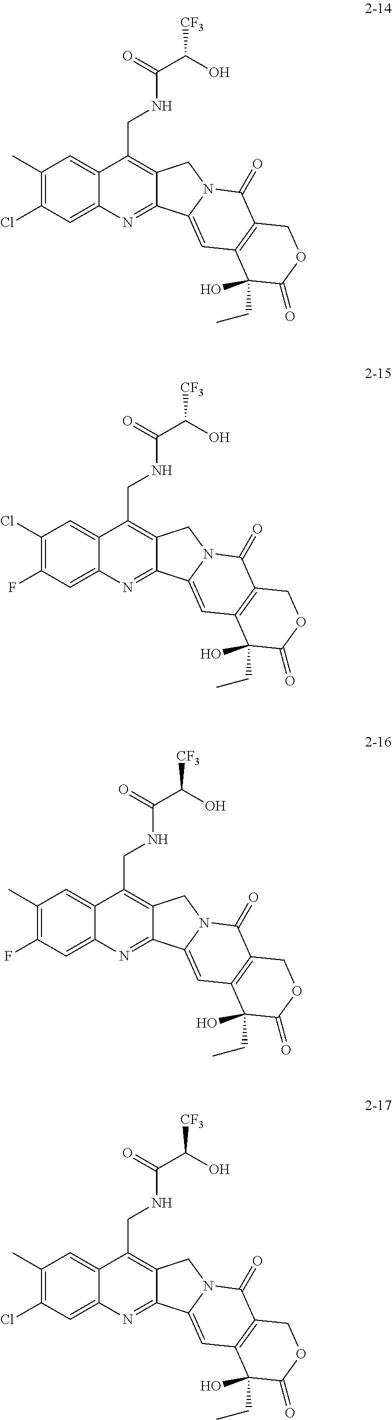 [SMILES: CC[C@@]1(O)C(=O)OCc2c1cc1n(c2=O)Cc2c-1nc1cc(Cl)c(C)cc1c2CNC(=O)[C@@H](O)C(F)(F)F ; CC[C@@]1(O)C(=O)OCc2c1cc1n(c2=O)Cc2c-1nc1cc(F)c(Cl)cc1c2CNC(=O)[C@@H](O)C(F)(F)F ; CC[C@@]1(O)C(=O)OCc2c1cc1n(c2=O)Cc2c-1nc1cc(F)c(C)cc1c2CNC(=O)[C@H](O)C(F)(F)F ; CC[C@@]1(O)C(=O)OCc2c1cc1n(c2=O)Cc2c-1nc1cc(Cl)c(C)cc1c2CNC(=O)[C@H](O)C(F)(F)F] 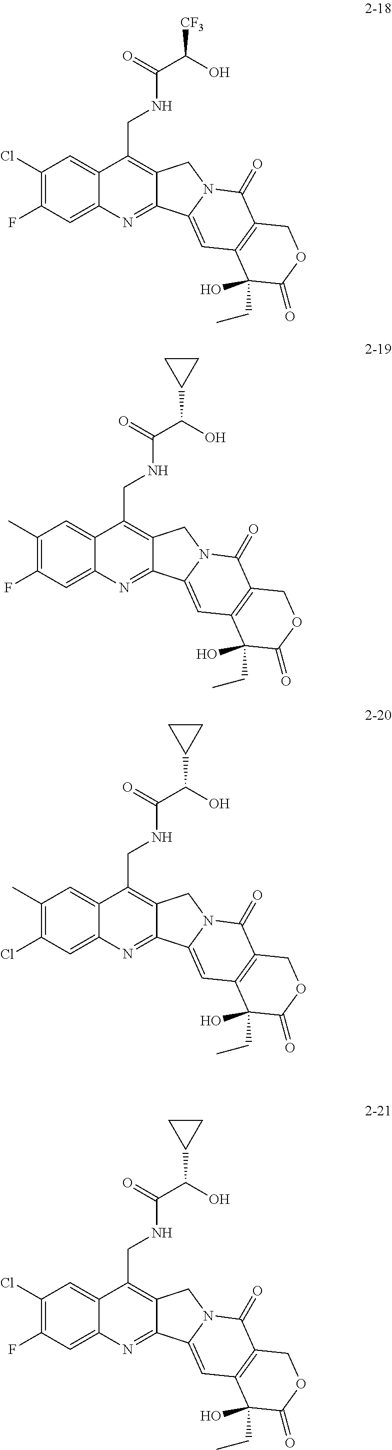 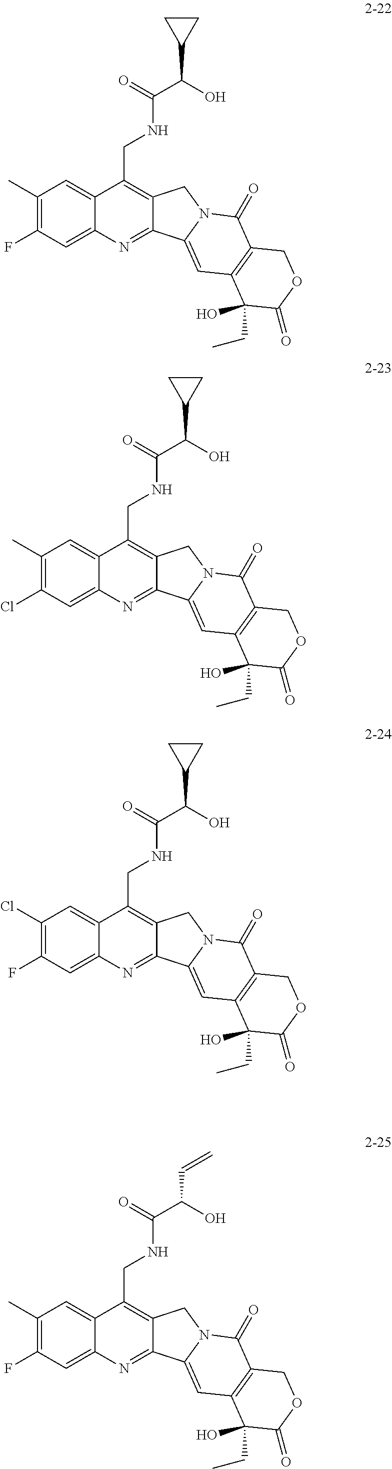 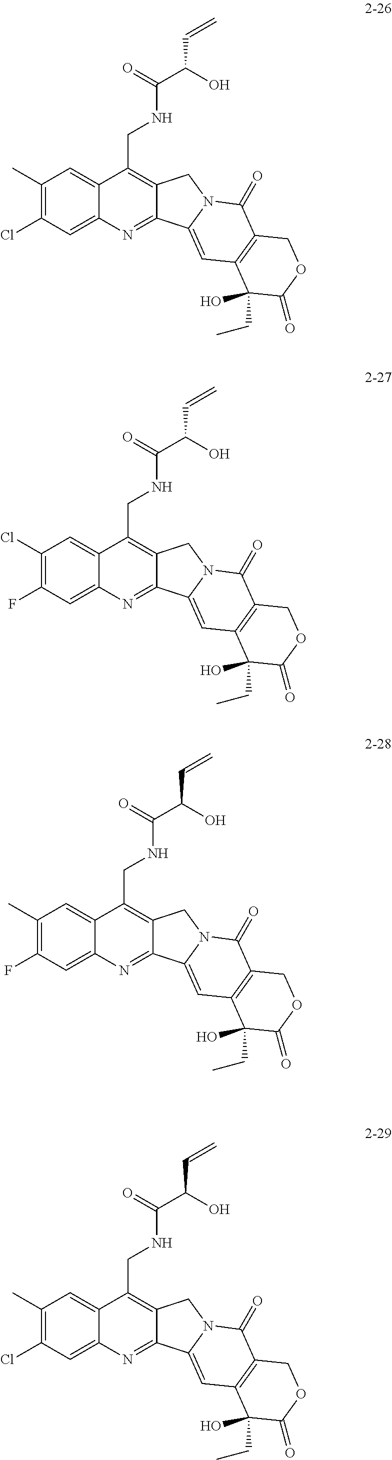 [SMILES: C=C[C@H](O)C(=O)NCc1c2c(nc3cc(Cl)c(C)cc13)-c1cc3c(c(=O)n1C2)COC(=O)[C@]3(O)CC ; C=C[C@H](O)C(=O)NCc1c2c(nc3cc(F)c(Cl)cc13)-c1cc3c(c(=O)n1C2)COC(=O)[C@]3(O)CC ; C=C[C@@H](O)C(=O)NCc1c2c(nc3cc(F)c(C)cc13)-c1cc3c(c(=O)n1C2)COC(=O)[C@]3(O)CC ; C=C[C@@H](O)C(=O)NCc1c2c(nc3cc(Cl)c(C)cc13)-c1cc3c(c(=O)n1C2)COC(=O)[C@]3(O)CC] 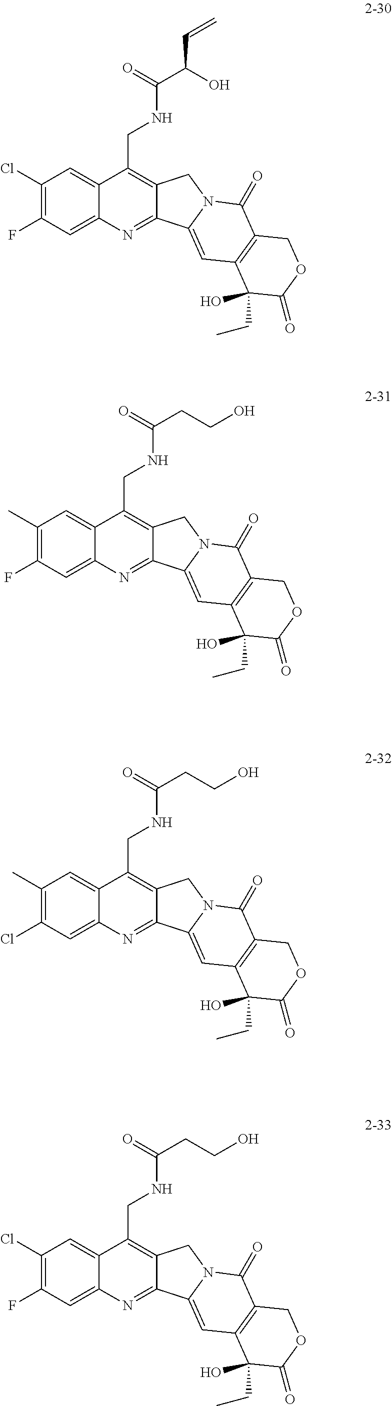 [SMILES: C=C[C@@H](O)C(=O)NCc1c2c(nc3cc(F)c(Cl)cc13)-c1cc3c(c(=O)n1C2)COC(=O)[C@]3(O)CC ; CC[C@@]1(O)C(=O)OCc2c1cc1n(c2=O)Cc2c-1nc1cc(F)c(C)cc1c2CNC(=O)CCO ; CC[C@@]1(O)C(=O)OCc2c1cc1n(c2=O)Cc2c-1nc1cc(Cl)c(C)cc1c2CNC(=O)CCO ; CC[C@@]1(O)C(=O)OCc2c1cc1n(c2=O)Cc2c-1nc1cc(F)c(Cl)cc1c2CNC(=O)CCO] 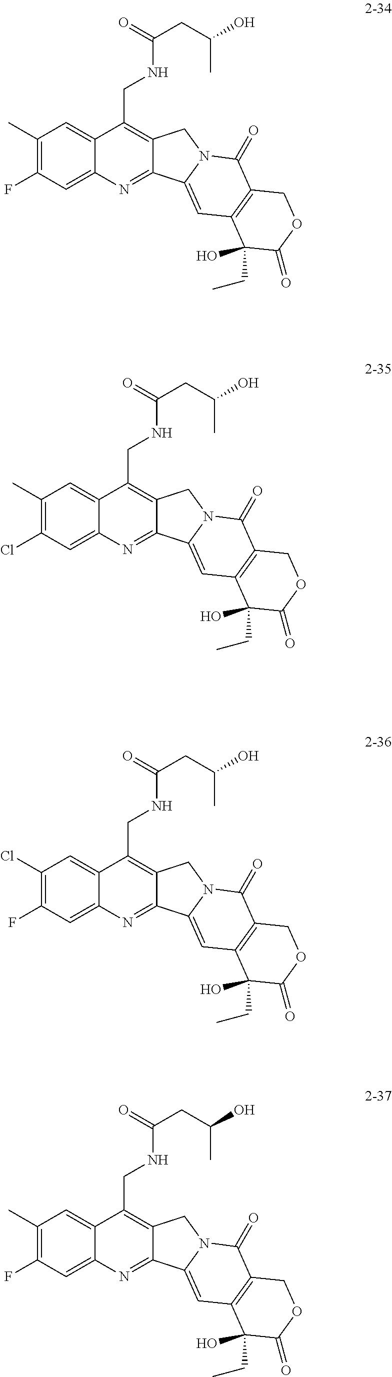 [SMILES: CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O] 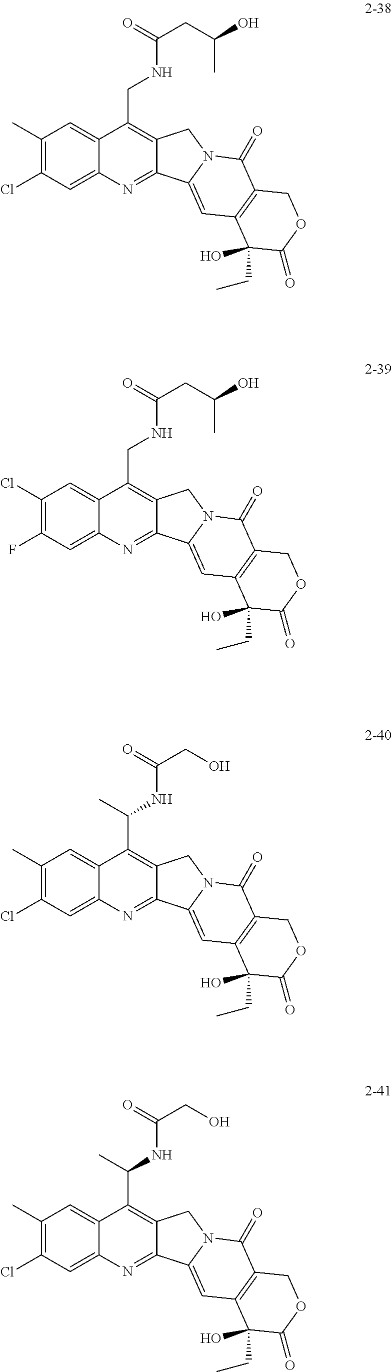 [SMILES: CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O ; CC[C@@]1(O)C(=O)OCc2c1cc1n(c2=O)Cc2c-1nc1cc(Cl)c(C)cc1c2[C@H](C)NC(=O)CO ; CC[C@@]1(O)C(=O)OCc2c1cc1n(c2=O)Cc2c-1nc1cc(Cl)c(C)cc1c2[C@@H](C)NC(=O)CO] 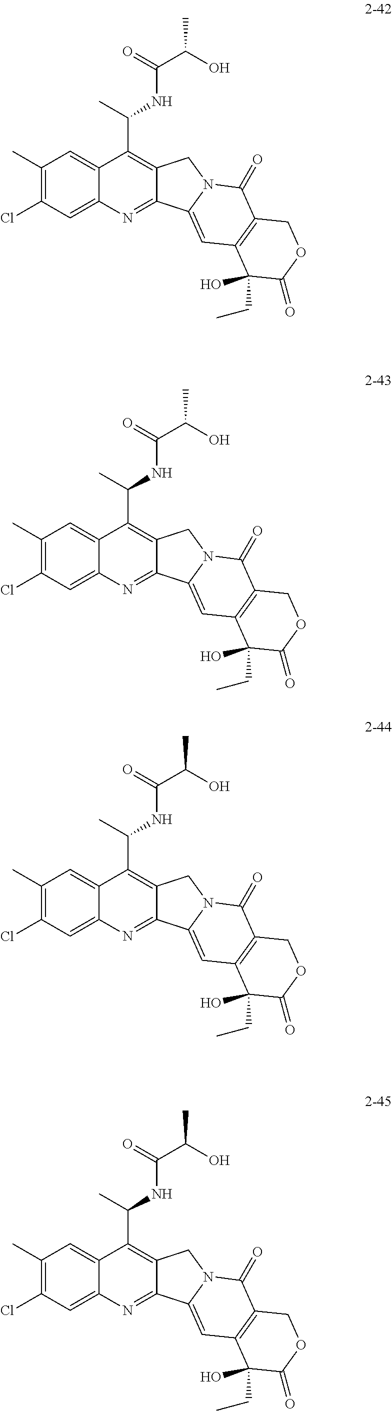 [SMILES: CC[C@@]1(O)C(=O)OCc2c1cc1n(c2=O)Cc2c-1nc1cc(Cl)c(C)cc1c2[C@H](C)NC(=O)[C@H](C)O ; CC[C@@]1(O)C(=O)OCc2c1cc1n(c2=O)Cc2c-1nc1cc(Cl)c(C)cc1c2[C@@H](C)NC(=O)[C@H](C)O ; CC[C@@]1(O)C(=O)OCc2c1cc1n(c2=O)Cc2c-1nc1cc(Cl)c(C)cc1c2[C@H](C)NC(=O)[C@@H](C)O ; CC[C@@]1(O)C(=O)OCc2c1cc1n(c2=O)Cc2c-1nc1cc(Cl)c(C)cc1c2[C@@H](C)NC(=O)[C@@H](C)O] 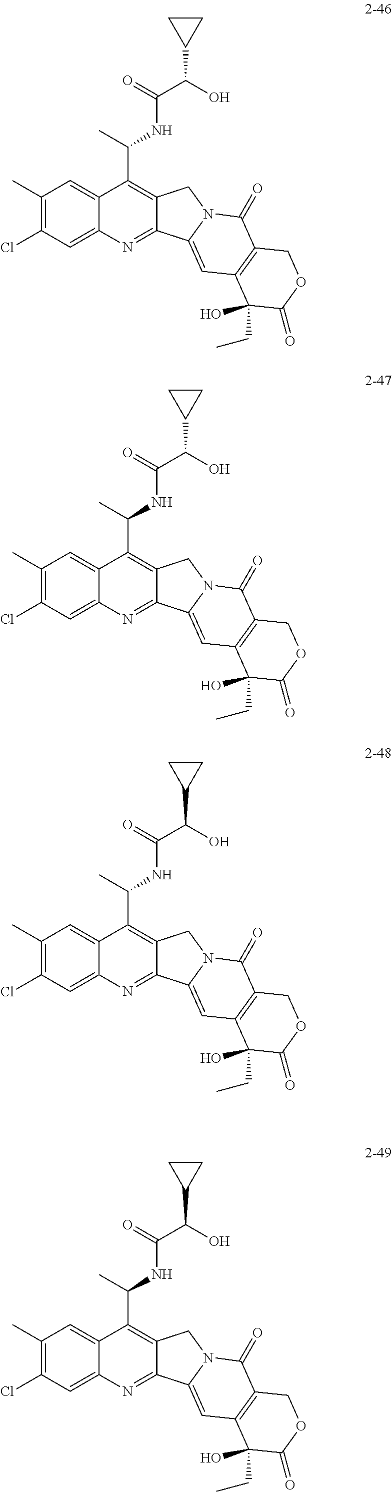 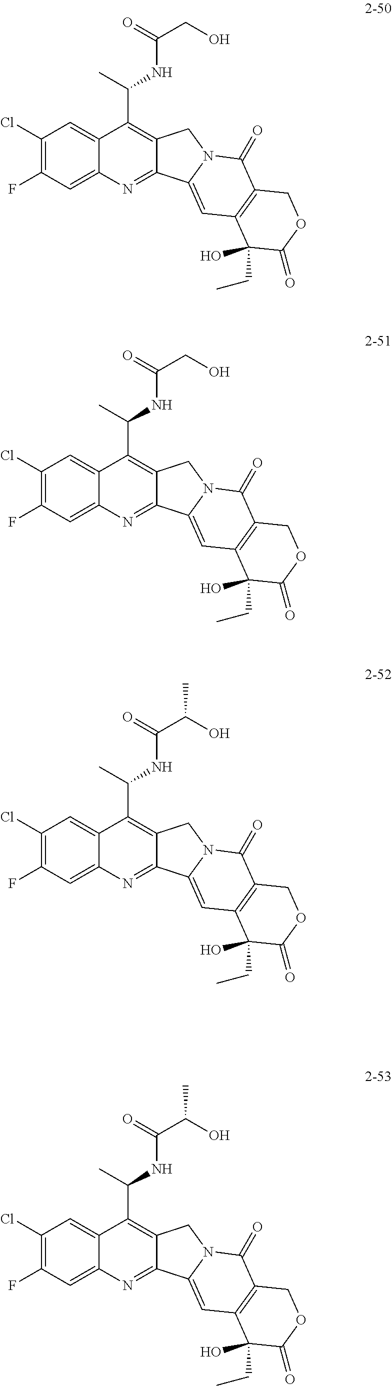 [SMILES: CC[C@@]1(O)C(=O)OCc2c1cc1n(c2=O)Cc2c-1nc1cc(F)c(Cl)cc1c2[C@H](C)NC(=O)CO ; CC[C@@]1(O)C(=O)OCc2c1cc1n(c2=O)Cc2c-1nc1cc(F)c(Cl)cc1c2[C@@H](C)NC(=O)CO ; CC[C@@]1(O)C(=O)OCc2c1cc1n(c2=O)Cc2c-1nc1cc(F)c(Cl)cc1c2[C@H](C)NC(=O)[C@H](C)O ; CC[C@@]1(O)C(=O)OCc2c1cc1n(c2=O)Cc2c-1nc1cc(F)c(Cl)cc1c2[C@@H](C)NC(=O)[C@H](C)O] 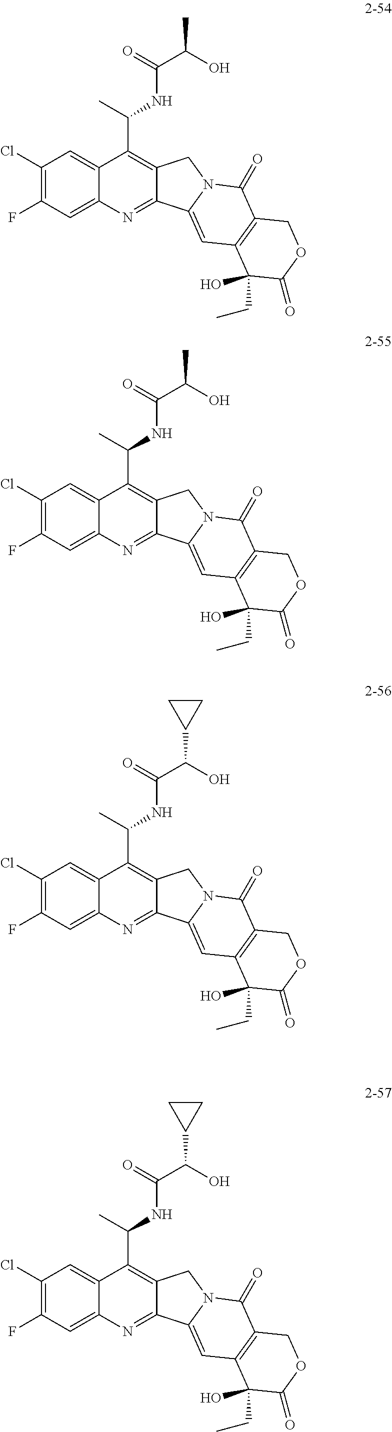 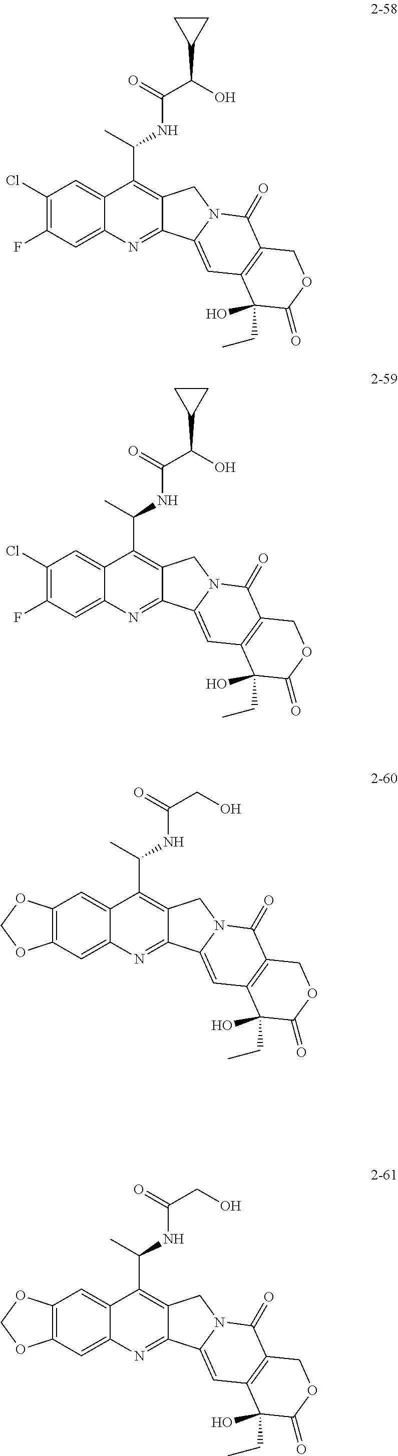 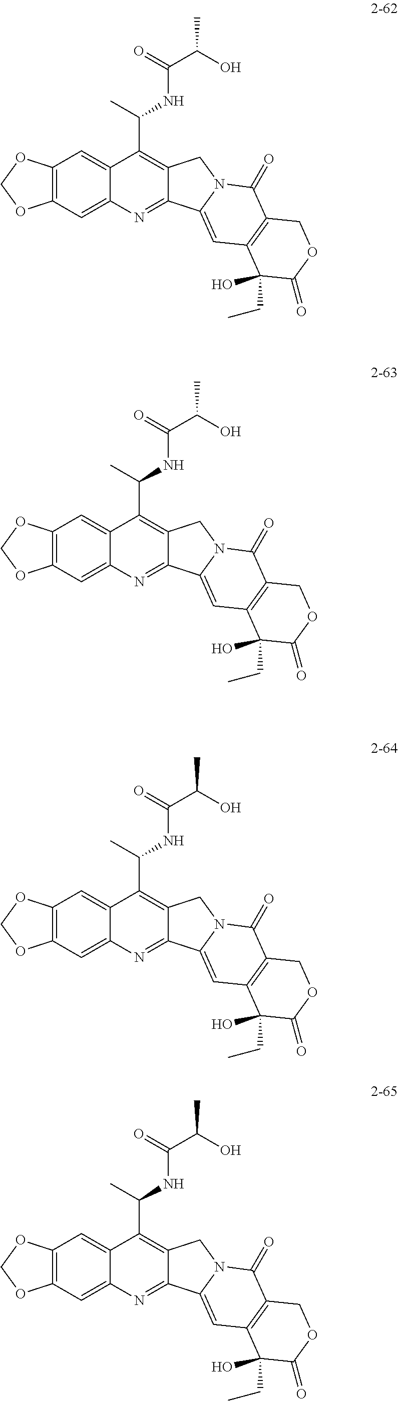 [SMILES: CC[C@@]1(O)C(=O)OCc2c1cc1n(c2=O)Cc2c-1nc1cc3c(cc1c2[C@H](C)NC(=O)[C@H](C)O)OCO3 ; CC[C@@]1(O)C(=O)OCc2c1cc1n(c2=O)Cc2c-1nc1cc3c(cc1c2[C@@H](C)NC(=O)[C@H](C)O)OCO3 ; CC[C@@]1(O)C(=O)OCc2c1cc1n(c2=O)Cc2c-1nc1cc3c(cc1c2[C@H](C)NC(=O)[C@@H](C)O)OCO3 ; CC[C@@]1(O)C(=O)OCc2c1cc1n(c2=O)Cc2c-1nc1cc3c(cc1c2[C@@H](C)NC(=O)[C@@H](C)O)OCO3] 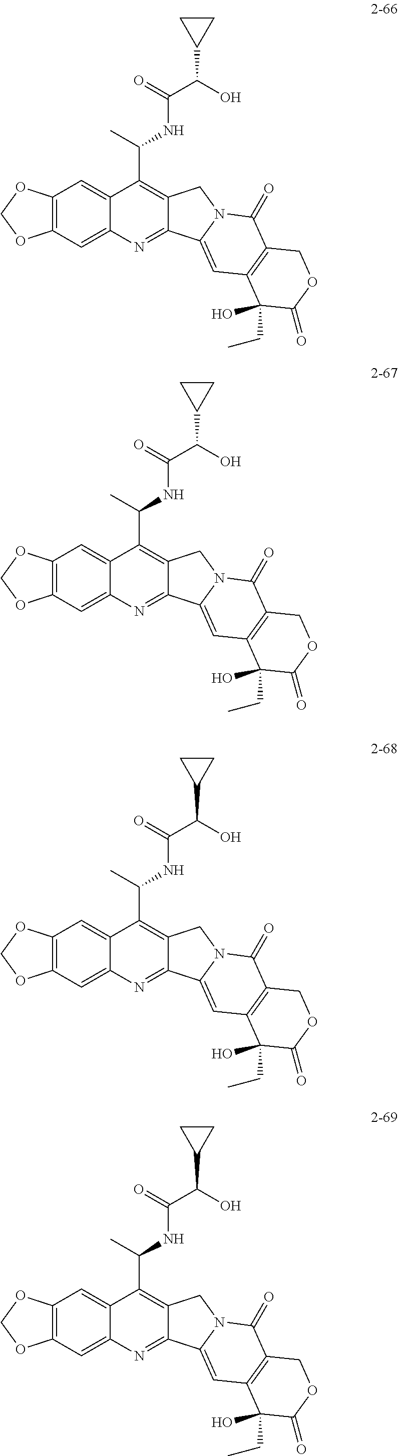 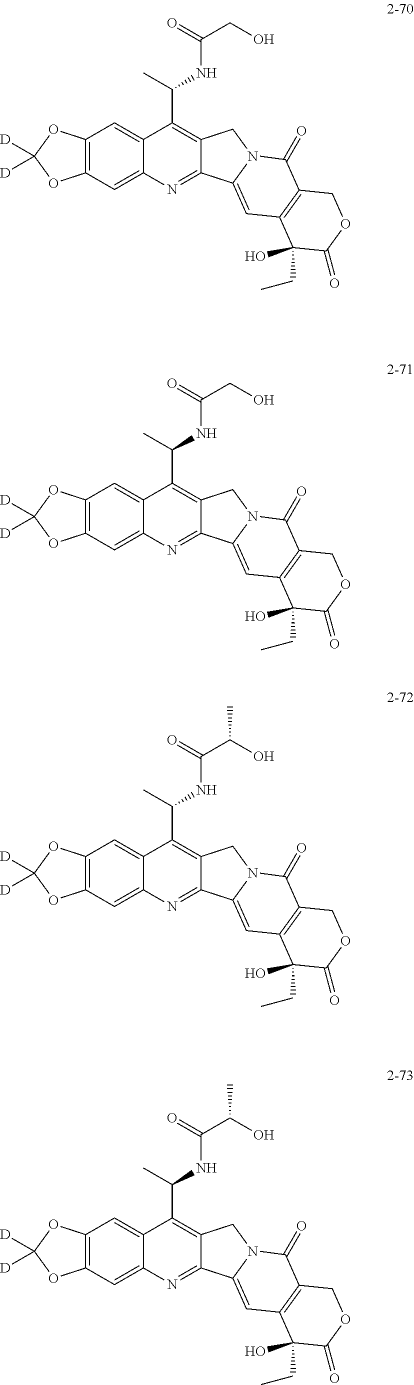 [SMILES: [2H]C1([2H])Oc2cc3nc4c(c([C@H](C)NC(=O)CO)c3cc2O1)Cn1c-4cc2c(c1=O)COC(=O)[C@]2(O)CC ; [2H]C1([2H])Oc2cc3nc4c(c([C@@H](C)NC(=O)CO)c3cc2O1)Cn1c-4cc2c(c1=O)COC(=O)[C@]2(O)CC ; [2H]C1([2H])Oc2cc3nc4c(c([C@H](C)NC(=O)[C@H](C)O)c3cc2O1)Cn1c-4cc2c(c1=O)COC(=O)[C@]2(O)CC ; [2H]C1([2H])Oc2cc3nc4c(c([C@@H](C)NC(=O)[C@H](C)O)c3cc2O1)Cn1c-4cc2c(c1=O)COC(=O)[C@]2(O)CC] 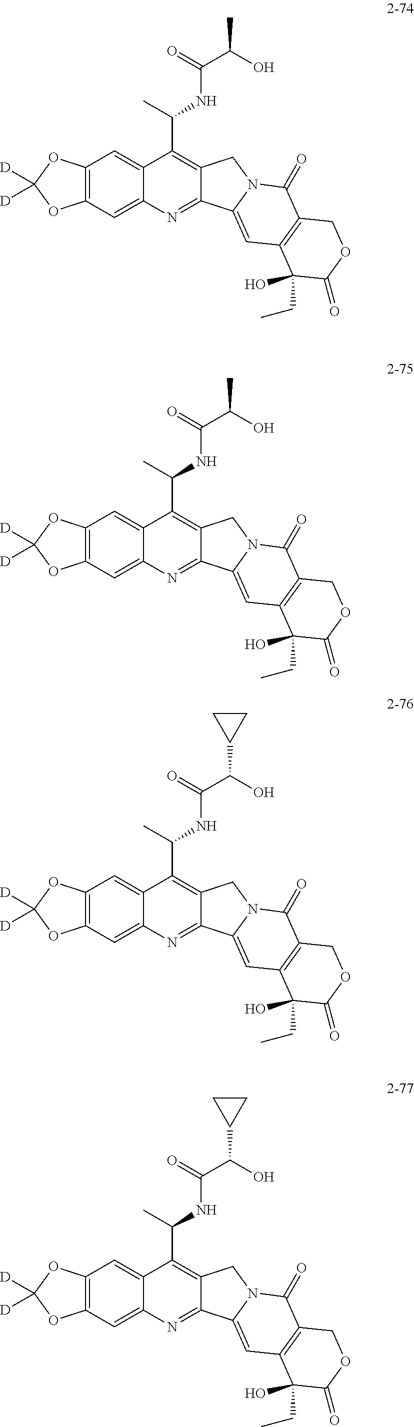 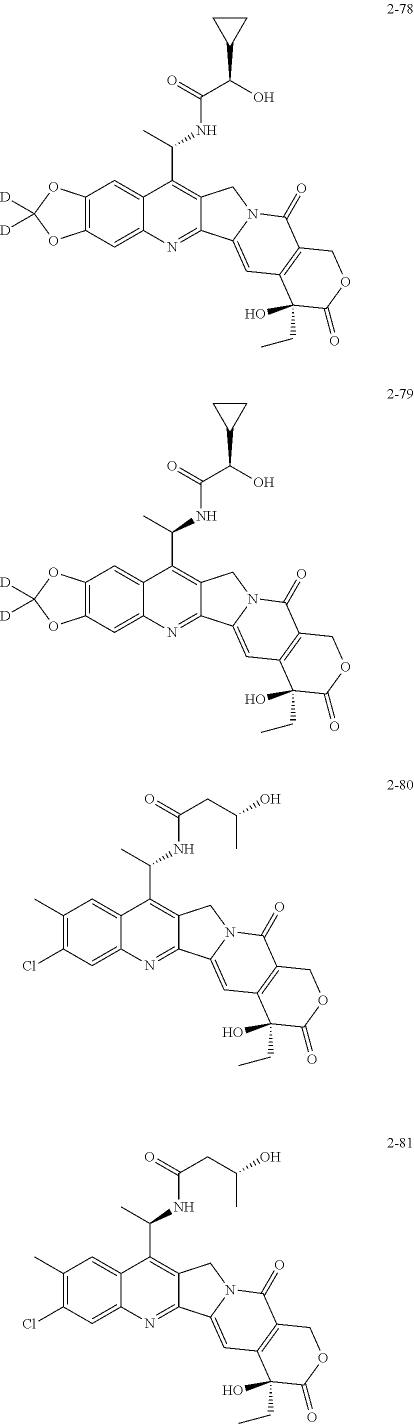 [SMILES: [2H]C1([2H])Oc2cc3nc4c(c([C@H](C)NC(=O)[C@H](O)C5CC5)c3cc2O1)Cn1c-4cc2c(c1=O)COC(=O)[C@]2(O)CC ; [2H]C1([2H])Oc2cc3nc4c(c([C@@H](C)NC(=O)[C@H](O)C5CC5)c3cc2O1)Cn1c-4cc2c(c1=O)COC(=O)[C@]2(O)CC ; CC[C@@]1(O)C(=O)OCc2c1cc1n(c2=O)Cc2c-1nc1cc(Cl)c(C)cc1c2[C@H](C)NC(=O)C[C@@H](C)O ; CC[C@@]1(O)C(=O)OCc2c1cc1n(c2=O)Cc2c-1nc1cc(Cl)c(C)cc1c2[C@@H](C)NC(=O)C[C@@H](C)O] 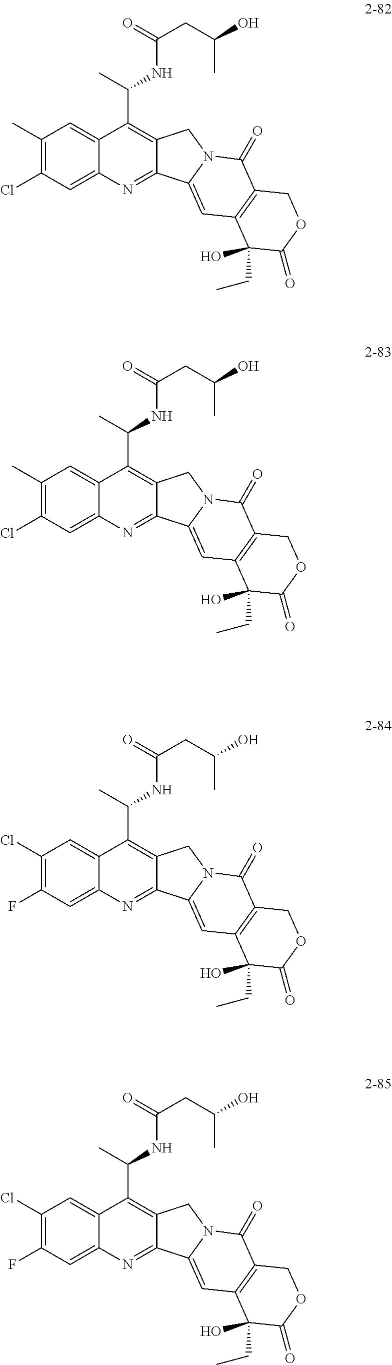 [SMILES: CC[C@@]1(O)C(=O)OCc2c1cc1n(c2=O)Cc2c-1nc1cc(Cl)c(C)cc1c2[C@H](C)NC(=O)C[C@H](C)O ; CC[C@@]1(O)C(=O)OCc2c1cc1n(c2=O)Cc2c-1nc1cc(Cl)c(C)cc1c2[C@@H](C)NC(=O)C[C@H](C)O ; CC[C@@]1(O)C(=O)OCc2c1cc1n(c2=O)Cc2c-1nc1cc(F)c(Cl)cc1c2[C@H](C)NC(=O)C[C@@H](C)O ; CC[C@@]1(O)C(=O)OCc2c1cc1n(c2=O)Cc2c-1nc1cc(F)c(Cl)cc1c2[C@@H](C)NC(=O)C[C@@H](C)O] 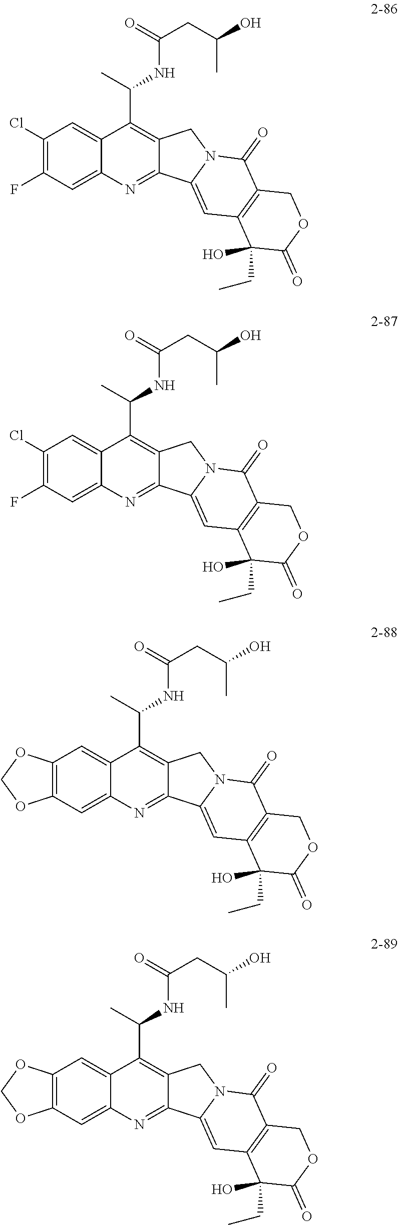 [SMILES: CC[C@@]1(O)C(=O)OCc2c1cc1n(c2=O)Cc2c-1nc1cc(F)c(Cl)cc1c2[C@H](C)NC(=O)C[C@H](C)O ; CC[C@@]1(O)C(=O)OCc2c1cc1n(c2=O)Cc2c-1nc1cc(F)c(Cl)cc1c2[C@@H](C)NC(=O)C[C@H](C)O ; CC[C@@]1(O)C(=O)OCc2c1cc1n(c2=O)Cc2c-1nc1cc3c(cc1c2[C@H](C)NC(=O)C[C@@H](C)O)OCO3 ; CC[C@@]1(O)C(=O)OCc2c1cc1n(c2=O)Cc2c-1nc1cc3c(cc1c2[C@@H](C)NC(=O)C[C@@H](C)O)OCO3] 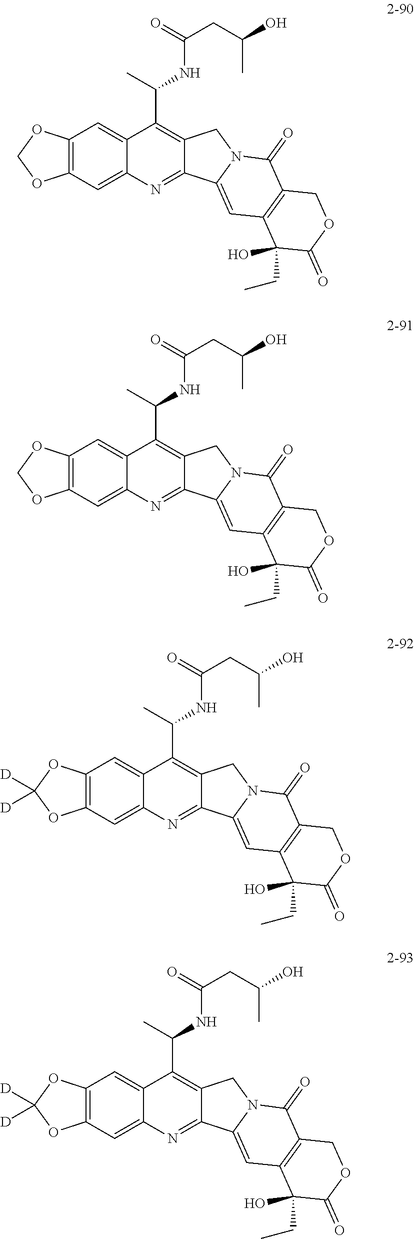 [SMILES: CC[C@@]1(O)C(=O)OCc2c1cc1n(c2=O)Cc2c-1nc1cc3c(cc1c2[C@H](C)NC(=O)C[C@H](C)O)OCO3 ; CC[C@@]1(O)C(=O)OCc2c1cc1n(c2=O)Cc2c-1nc1cc3c(cc1c2[C@@H](C)NC(=O)C[C@H](C)O)OCO3 ; [2H]C1([2H])Oc2cc3nc4c(c([C@H](C)NC(=O)C[C@@H](C)O)c3cc2O1)Cn1c-4cc2c(c1=O)COC(=O)[C@]2(O)CC ; [2H]C1([2H])Oc2cc3nc4c(c([C@@H](C)NC(=O)C[C@@H](C)O)c3cc2O1)Cn1c-4cc2c(c1=O)COC(=O)[C@]2(O)CC] 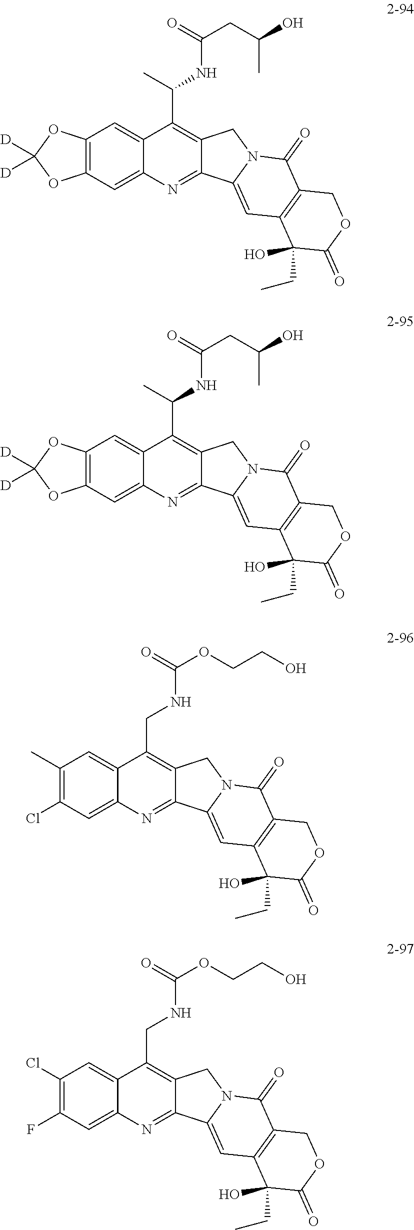 [SMILES: [2H]C1([2H])Oc2cc3nc4c(c([C@H](C)NC(=O)C[C@H](C)O)c3cc2O1)Cn1c-4cc2c(c1=O)COC(=O)[C@]2(O)CC ; [2H]C1([2H])Oc2cc3nc4c(c([C@@H](C)NC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1cc(Cl)c(C)cc1c2CNC(=O)OCCO ; CC[C@@]1(O)C(=O)OCc2c1cc1n(c2=O)Cc2c-1nc1cc(F)c(Cl)cc1c2CNC(=O)OCCO] 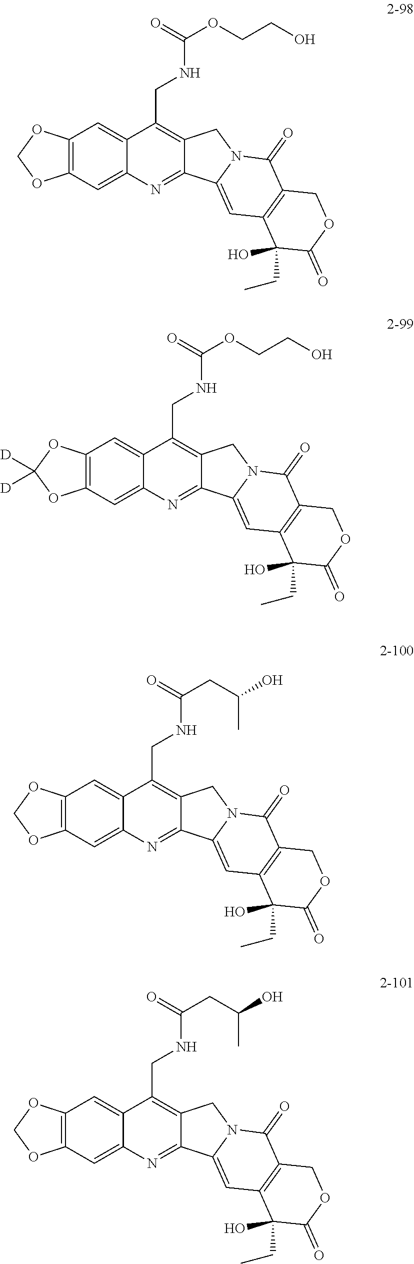 [SMILES: CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC ; CC[C@@]1(O)C(=O)OCc2c1cc1n(c2=O)Cc2c-1nc1cc3c(cc1c2CNC(=O)C[C@@H](C)O)OCO3 ; CC[C@@]1(O)C(=O)OCc2c1cc1n(c2=O)Cc2c-1nc1cc3c(cc1c2CNC(=O)C[C@H](C)O)OCO3] 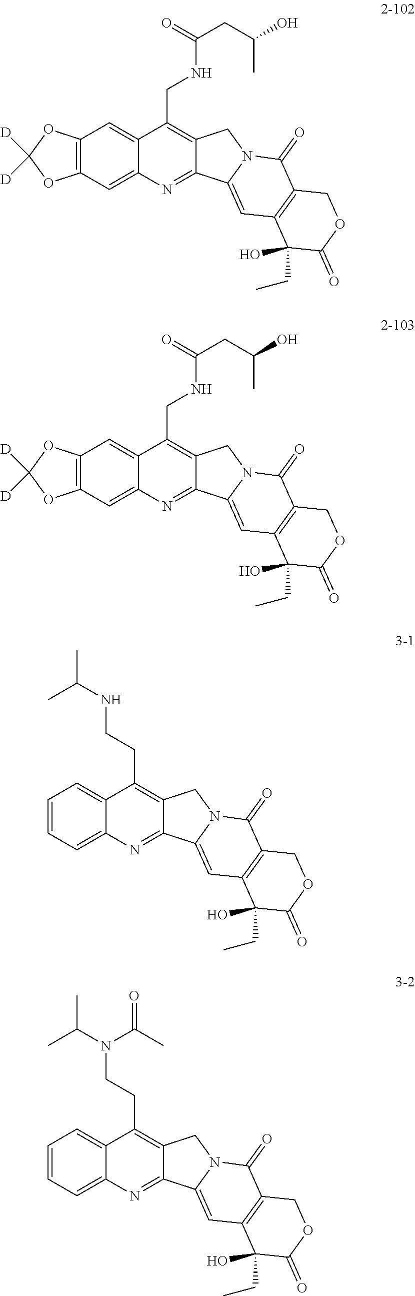 [SMILES: [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C] 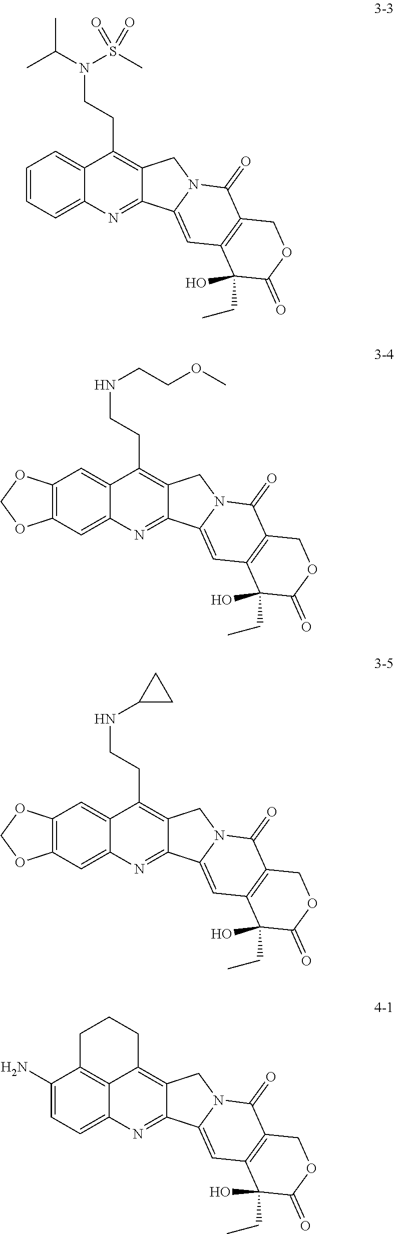 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3 ; CC[C@@]1(O)C(=O)OCc2c1cc1n(c2=O)Cc2c-1nc1cc3c(cc1c2CCNC1CC1)OCO3 ; CC[C@@]1(O)C(=O)OCc2c1cc1n(c2=O)Cc2c-1nc1ccc(N)c3c1c2CCC3] 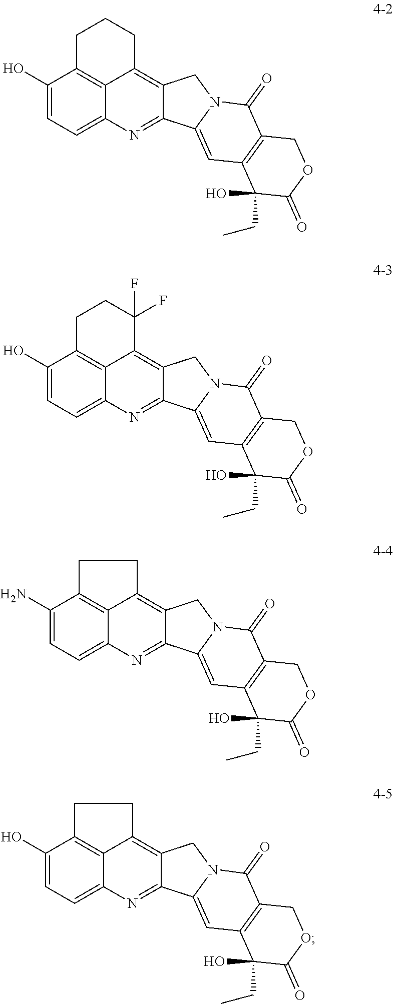 [SMILES: CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2C(F)(F)CC3 ; CC[C@@]1(O)C(=O)OCc2c1cc1n(c2=O)Cc2c-1nc1ccc(N)c3c1c2CC3 ; CC[C@@]1(O)C(=O)OCc2c1cc1n(c2=O)Cc2c-1nc1ccc(O)c3c1c2CC3]
preferably, the cytotoxic drug is selected from the group consisting of the following compounds or isotopically labeled compounds thereof: 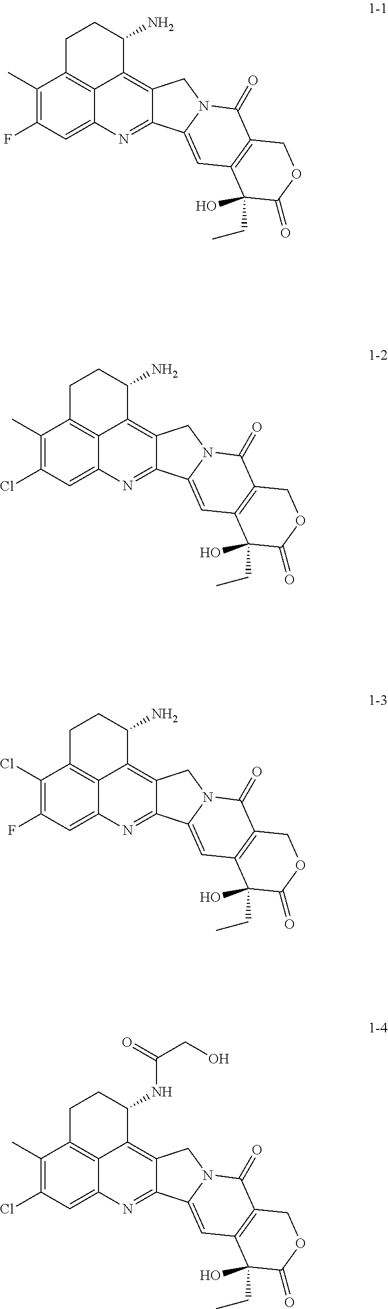 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3] 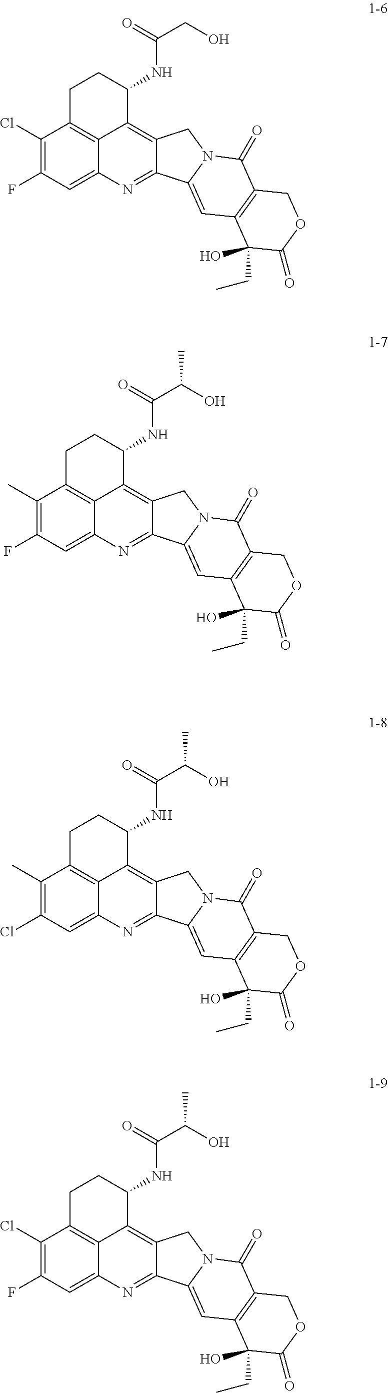 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3] 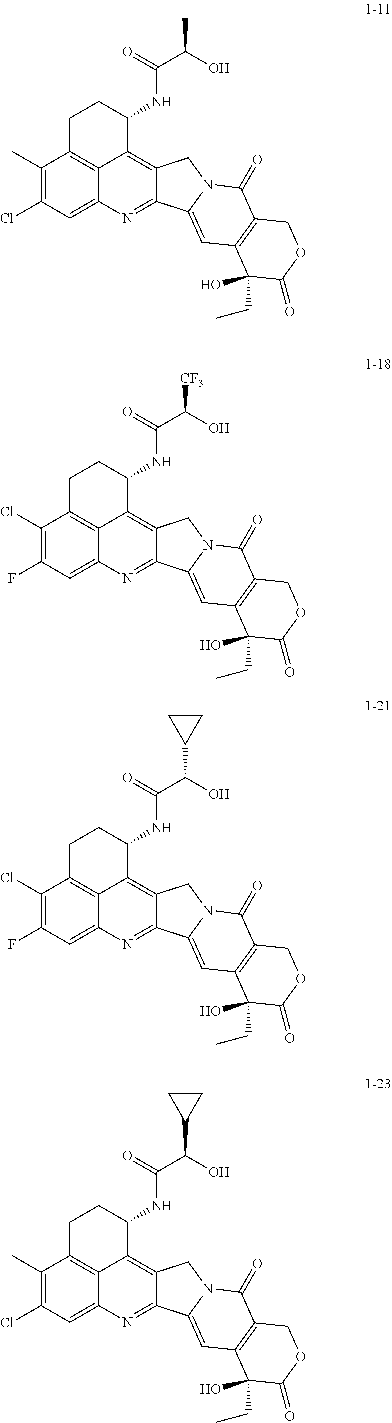 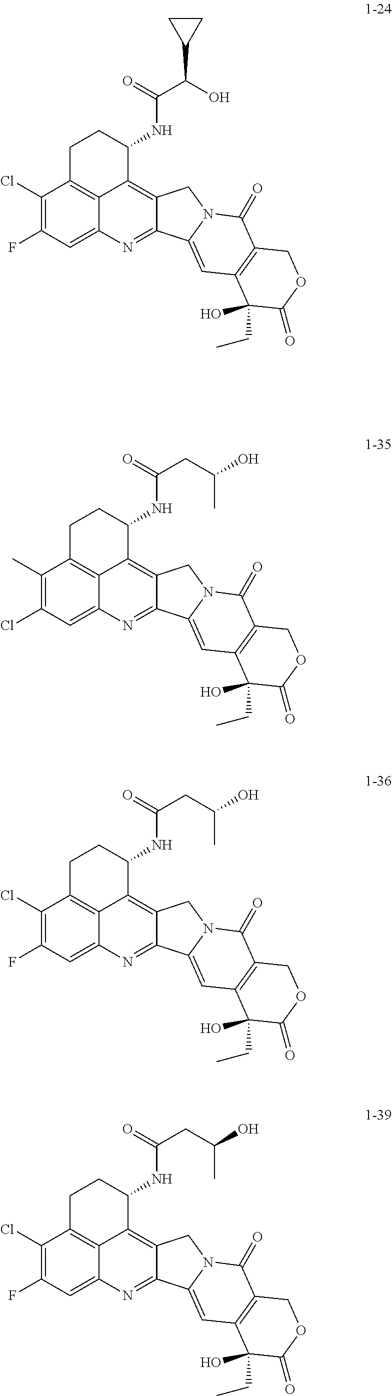 [SMILES: CC[C@@]1(O)C(=O)OCc2c1cc1n(c2=O)Cc2c-1nc1cc(F)c(Cl)c3c1c2[C@@H](NC(=O)[C@H](O)C1CC1)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3] 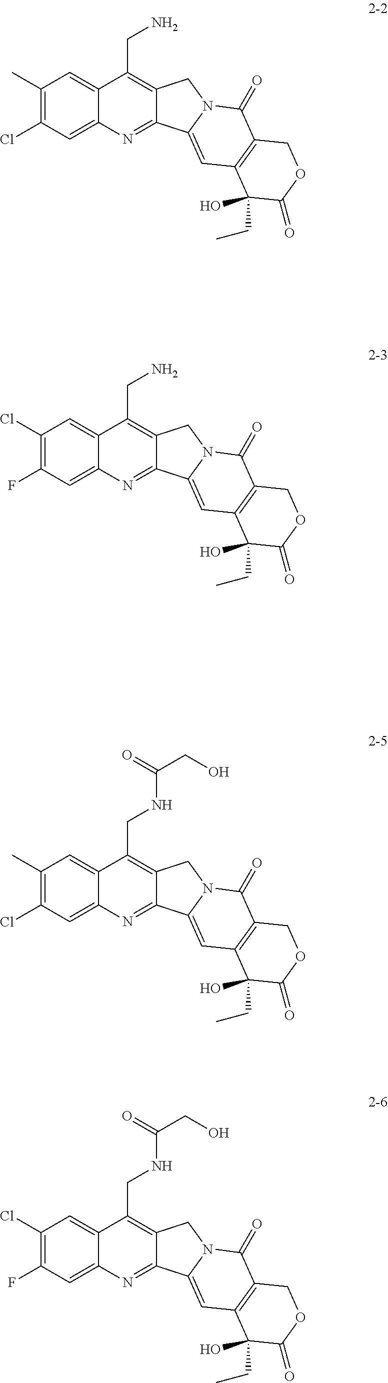 [SMILES: CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO] 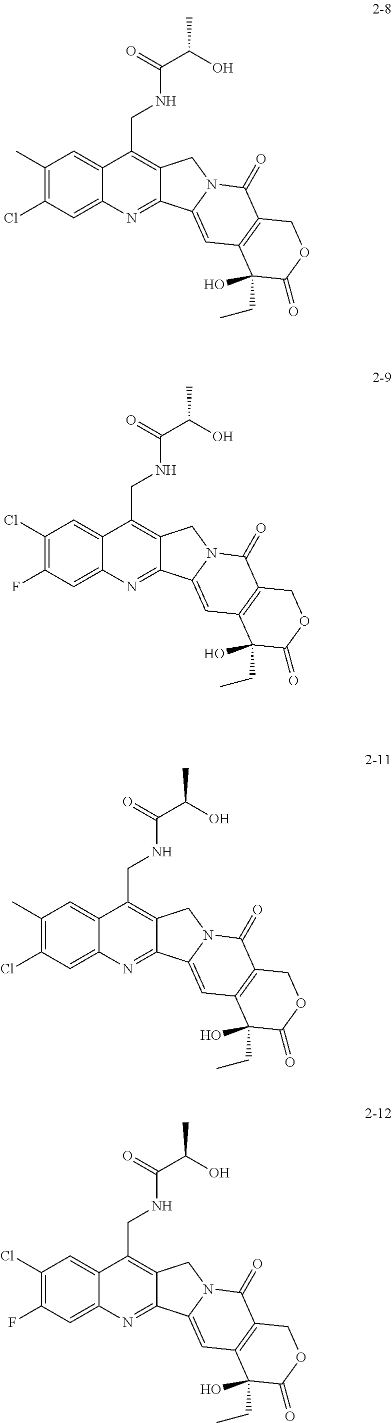 [SMILES: CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O] 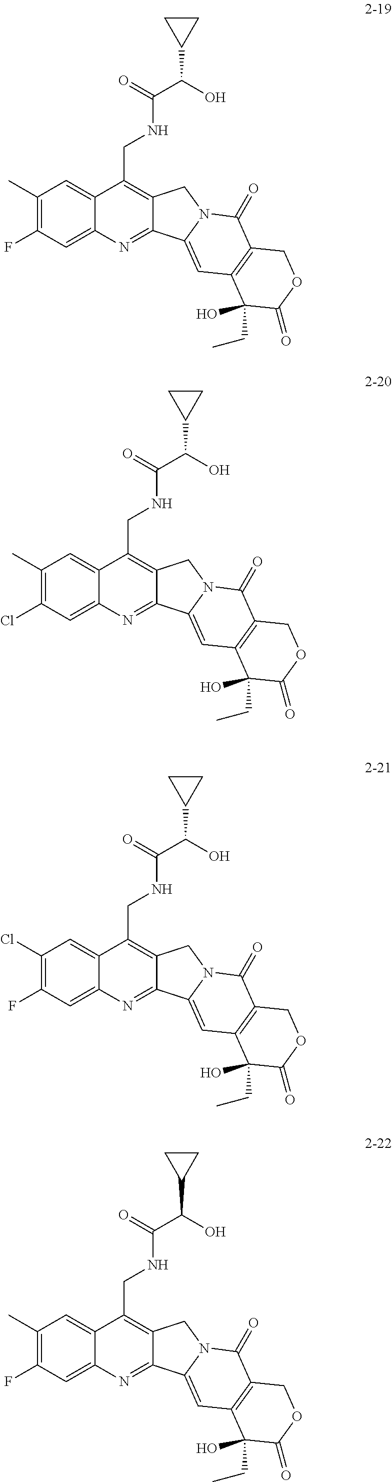 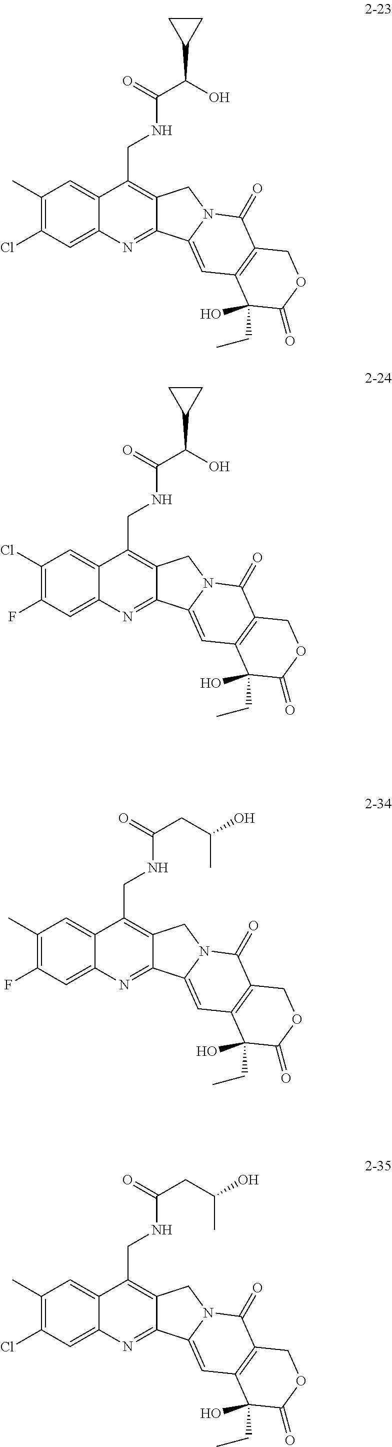 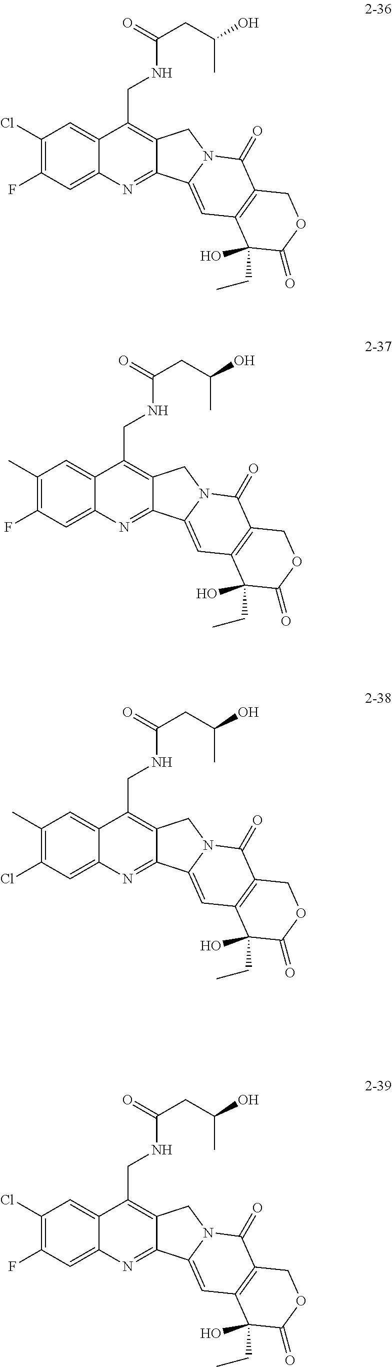 [SMILES: CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O] 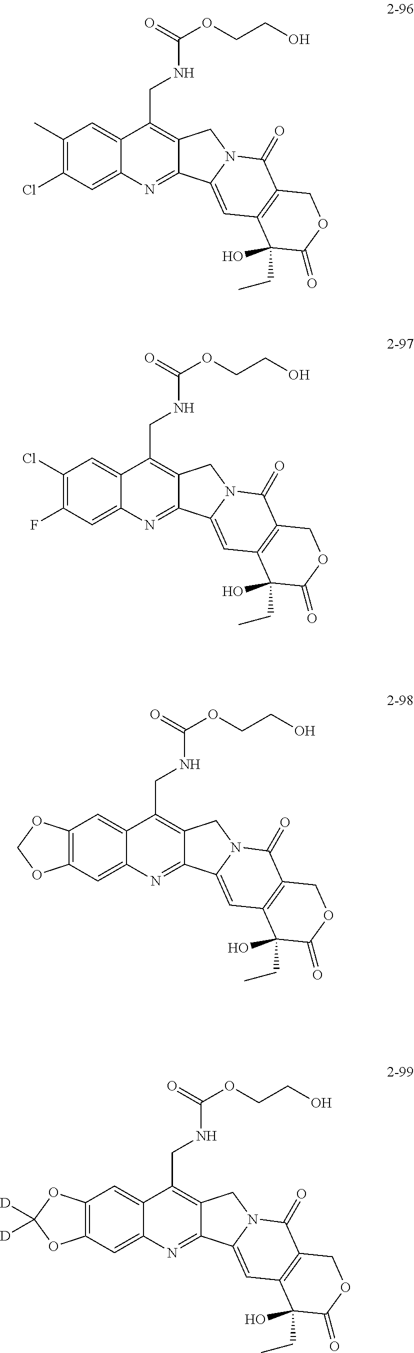 [SMILES: CC[C@@]1(O)C(=O)OCc2c1cc1n(c2=O)Cc2c-1nc1cc(Cl)c(C)cc1c2CNC(=O)OCCO ; CC[C@@]1(O)C(=O)OCc2c1cc1n(c2=O)Cc2c-1nc1cc(F)c(Cl)cc1c2CNC(=O)OCCO ; CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC] 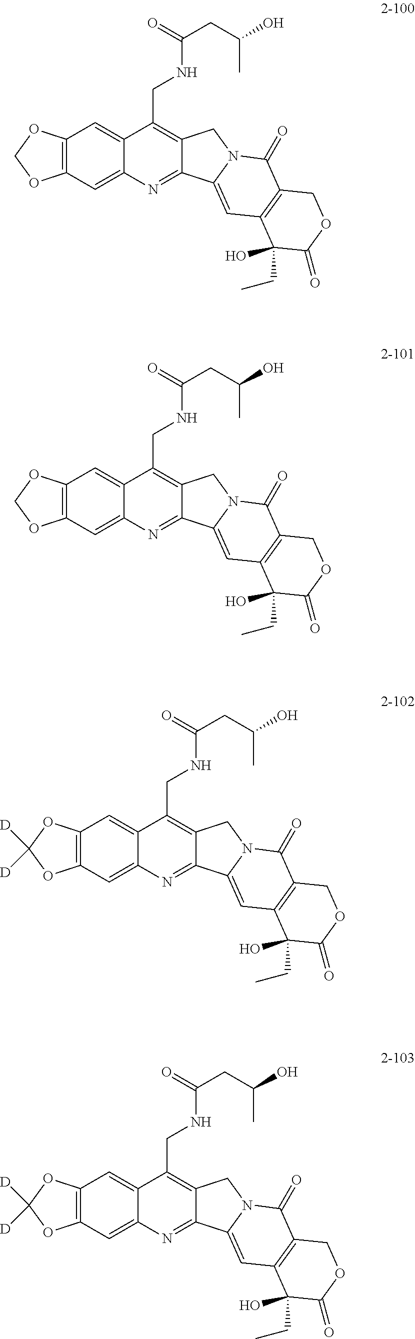 [SMILES: CC[C@@]1(O)C(=O)OCc2c1cc1n(c2=O)Cc2c-1nc1cc3c(cc1c2CNC(=O)C[C@@H](C)O)OCO3 ; CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC] 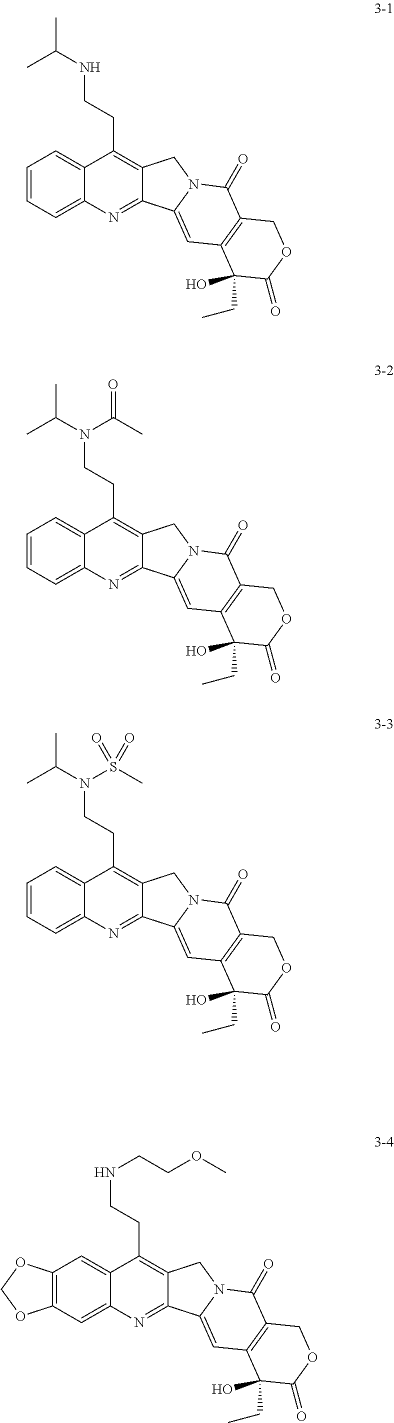 [SMILES: CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3] 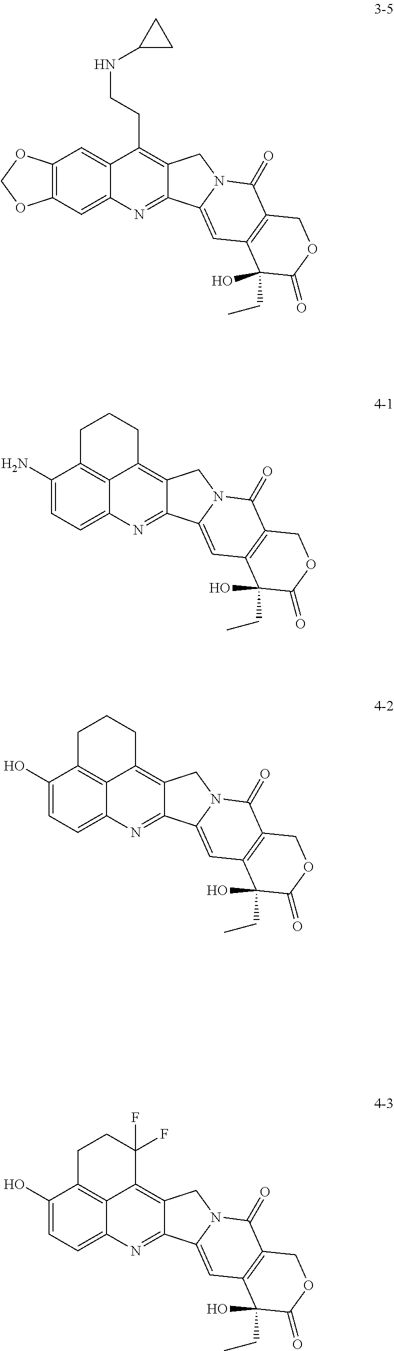 [SMILES: CC[C@@]1(O)C(=O)OCc2c1cc1n(c2=O)Cc2c-1nc1cc3c(cc1c2CCNC1CC1)OCO3 ; CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3 ; CC[C@@]1(O)C(=O)OCc2c1cc1n(c2=O)Cc2c-1nc1ccc(O)c3c1c2C(F)(F)CC3] 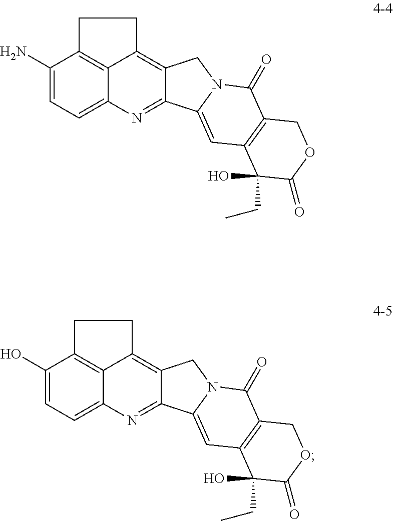 [SMILES: CC[C@@]1(O)C(=O)OCc2c1cc1n(c2=O)Cc2c-1nc1ccc(N)c3c1c2CC3 ; CC[C@@]1(O)C(=O)OCc2c1cc1n(c2=O)Cc2c-1nc1ccc(O)c3c1c2CC3]
preferably, the cytotoxic drug is selected from the group consisting of the following compounds or isotopically labeled compounds thereof: 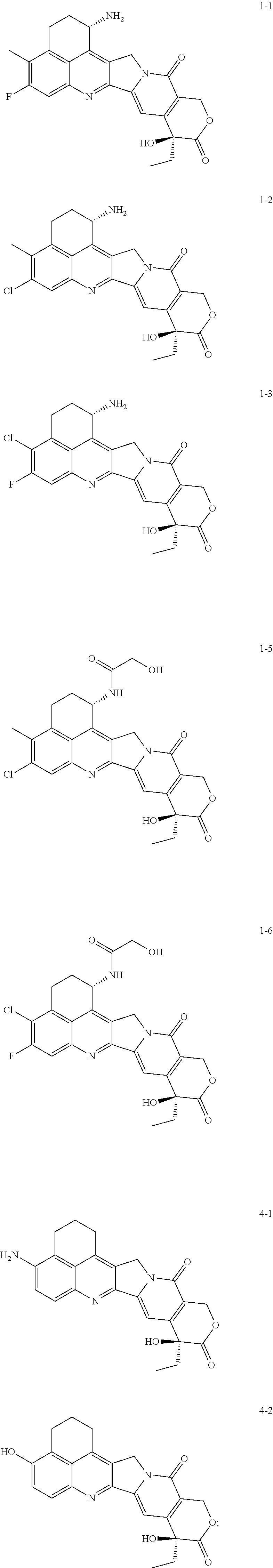
preferably, the cytotoxic drug is selected from the group consisting of the following compounds or isotopically labeled compounds thereof: 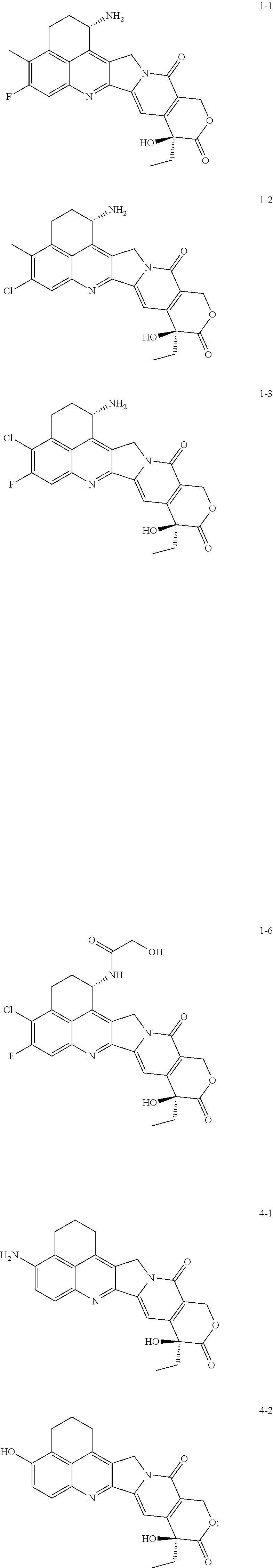 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1ccc(N)c3c1c2CCC3 ; CC[C@@]1(O)C(=O)OCc2c1cc1n(c2=O)Cc2c-1nc1ccc(O)c3c1c2CCC3]
preferably, the cytotoxic drug is selected from the group consisting of the following compounds or isotopically labeled compounds thereof: 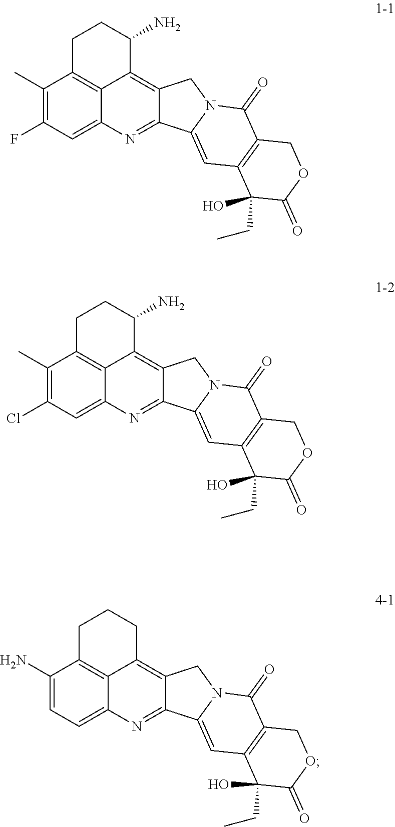 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1ccc(N)c3c1c2CCC3]
preferably, the cytotoxic drug is selected from the group consisting of the following compound or isotopically labeled compound thereof: 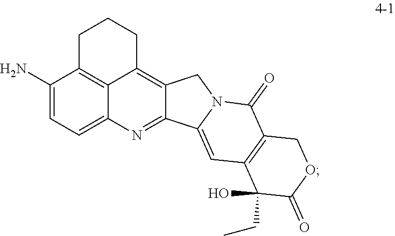 [SMILES: CC[C@@]1(O)C(=O)OCc2c1cc1n(c2=O)Cc2c-1nc1ccc(N)c3c1c2CCC3]
or, the cytotoxic drug is selected from the group consisting of the following compounds or isotopically labeled compounds thereof: 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38, 1-39, 2-1, 2-2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 2-80, 2-81, 2-82, 2-83, 2-84, 2-85, 2-86, 2-87, 2-88, 2-89, 2-90, 2-91, 2-92, 2-93, 2-94, 2-95, 2-96, 2-97, 2-98, 2-99, 2-100, 2-101, 2-102, 2-103, 3-1, 3-2, 3-3, 3-4, 3-5,
preferably, the cytotoxic drug is selected from the group consisting of the following compounds or isotopically labeled compounds thereof: 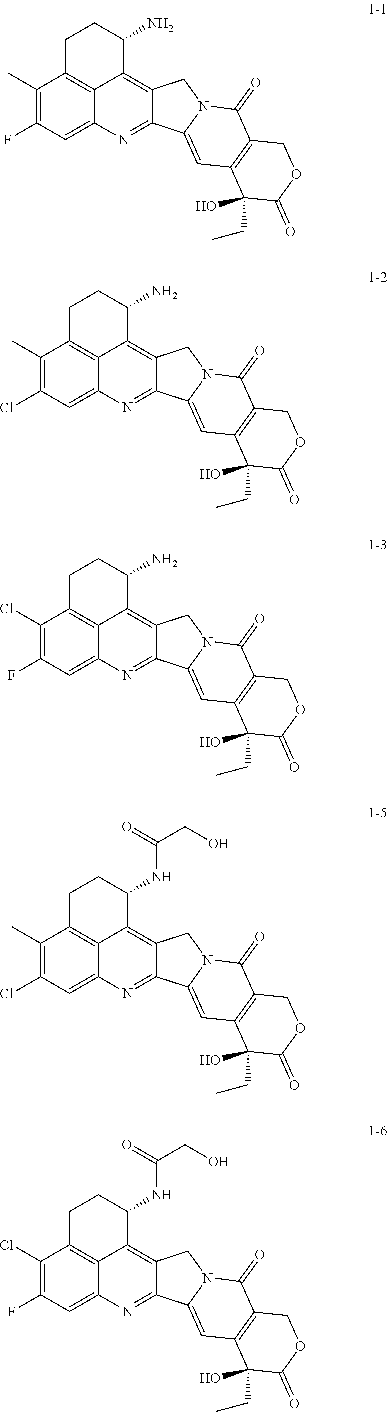 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3] 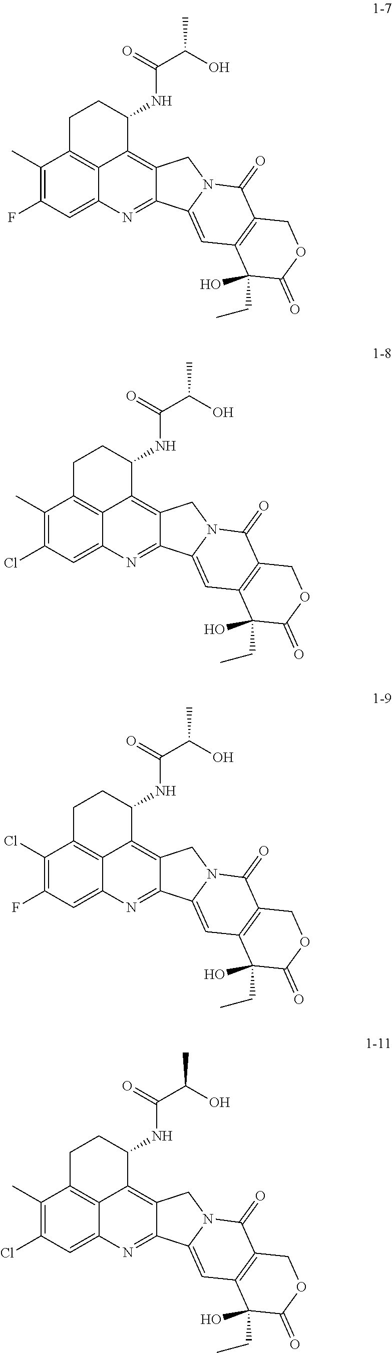 [SMILES: CC[C@@]1(O)C(=O)OCc2c1cc1n(c2=O)Cc2c-1nc1cc(F)c(C)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@H](C)O)CC3 ; CC[C@@]1(O)C(=O)OCc2c1cc1n(c2=O)Cc2c-1nc1cc(F)c(Cl)c3c1c2[C@@H](NC(=O)[C@H](C)O)CC3 ; CC[C@@]1(O)C(=O)OCc2c1cc1n(c2=O)Cc2c-1nc1cc(Cl)c(C)c3c1c2[C@@H](NC(=O)[C@@H](C)O)CC3] 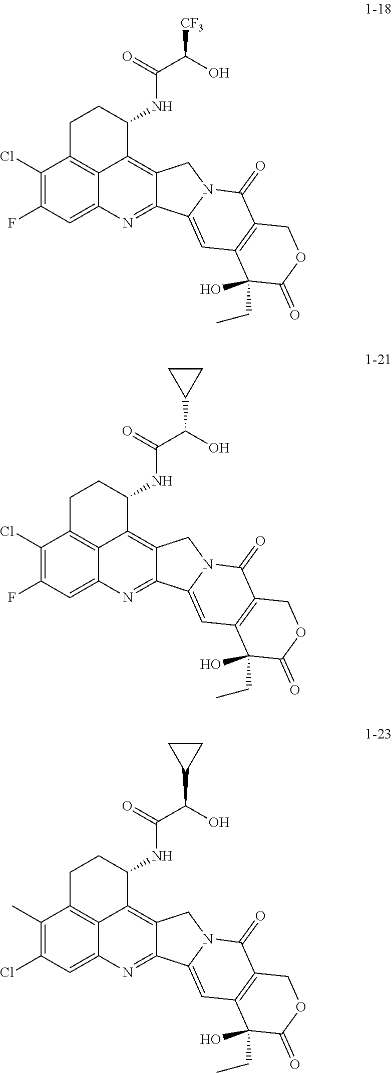 [SMILES: CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C1CC1)CC3 ; CC[C@@]1(O)C(=O)OCc2c1cc1n(c2=O)Cc2c-1nc1cc(Cl)c(C)c3c1c2[C@@H](NC(=O)[C@H](O)C1CC1)CC3] 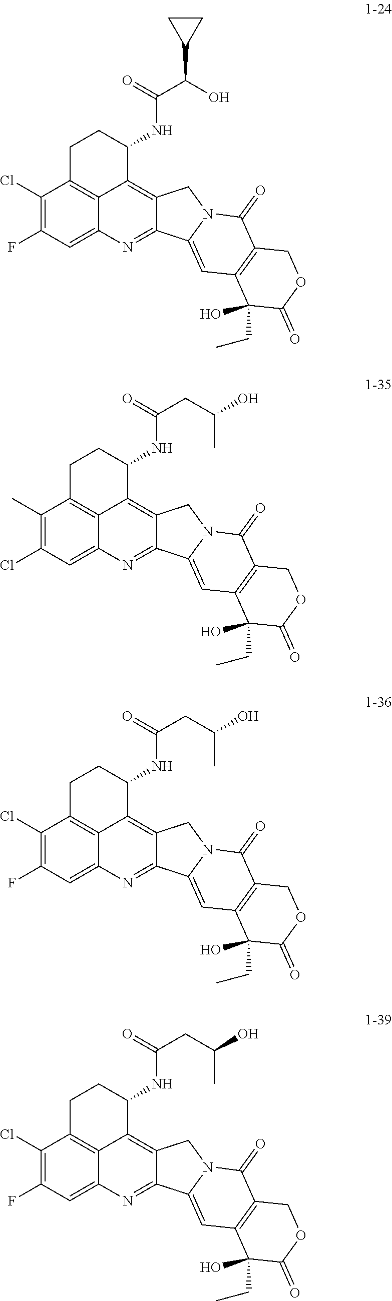 [SMILES: CC[C@@]1(O)C(=O)OCc2c1cc1n(c2=O)Cc2c-1nc1cc(F)c(Cl)c3c1c2[C@@H](NC(=O)[C@H](O)C1CC1)CC3 ; CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3] 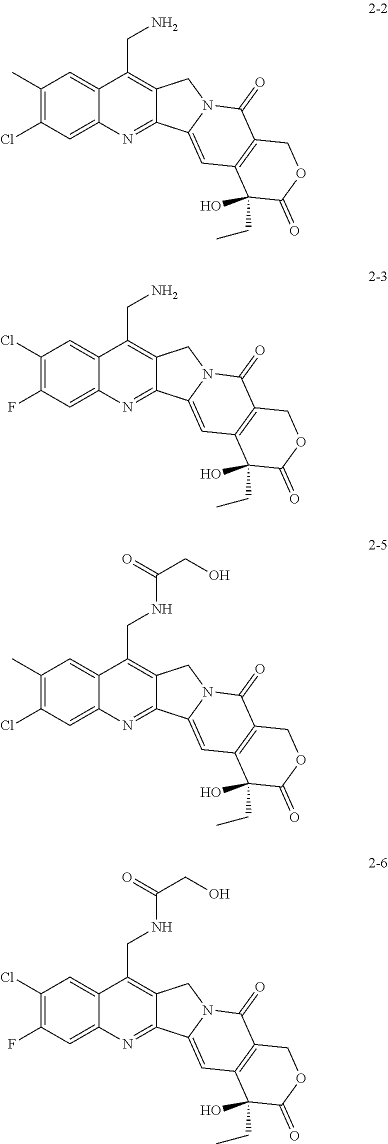 [SMILES: CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO] 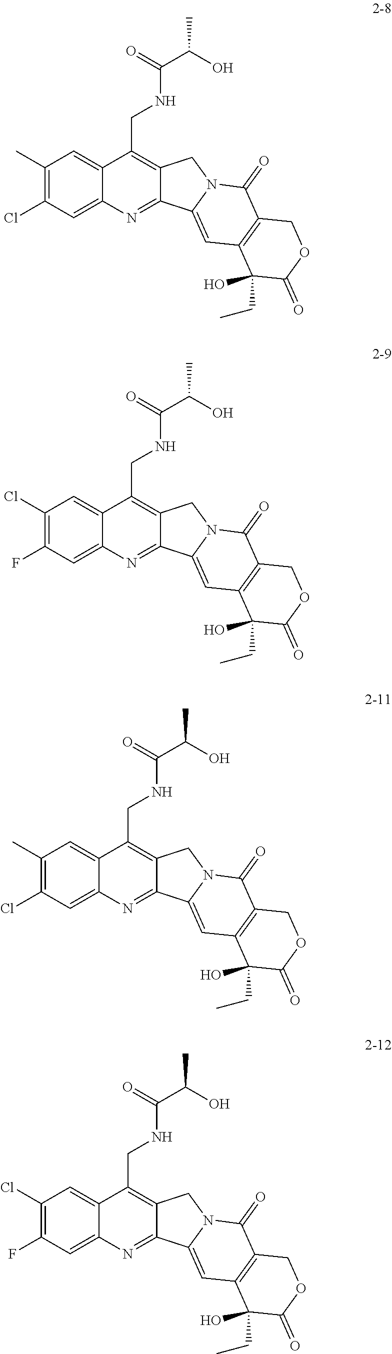 [SMILES: CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O] 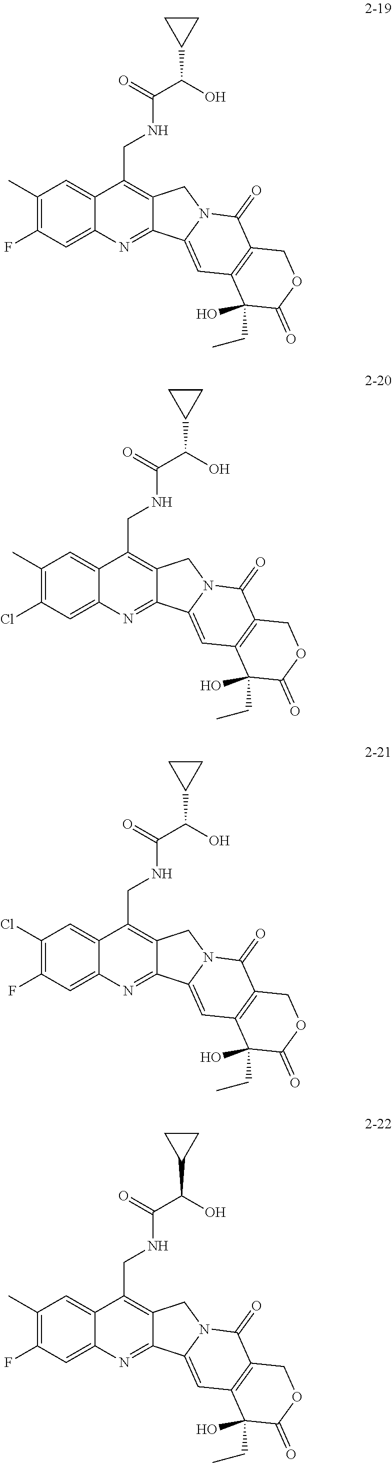 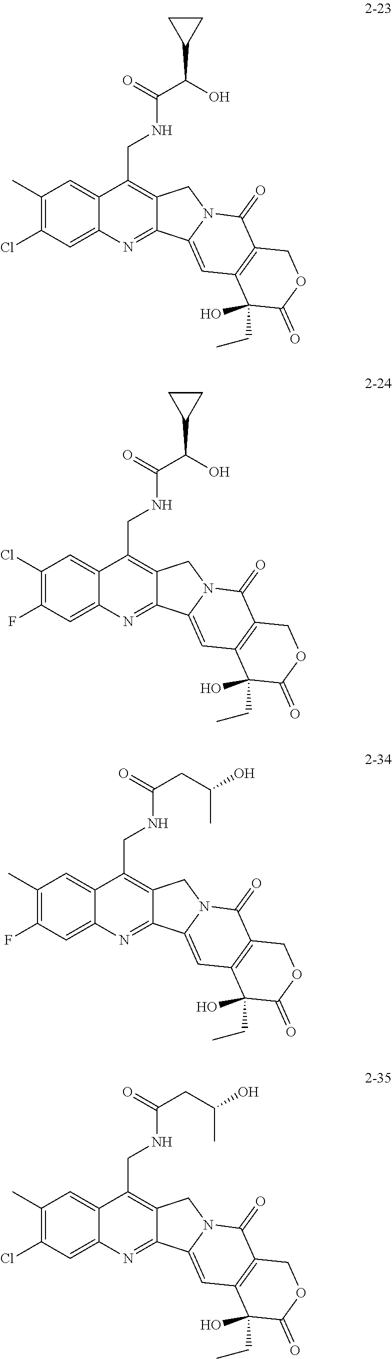 [SMILES: CC[C@@]1(O)C(=O)OCc2c1cc1n(c2=O)Cc2c-1nc1cc(Cl)c(C)cc1c2CNC(=O)[C@H](O)C1CC1 ; CC[C@@]1(O)C(=O)OCc2c1cc1n(c2=O)Cc2c-1nc1cc(F)c(Cl)cc1c2CNC(=O)[C@H](O)C1CC1 ; CC[C@@]1(O)C(=O)OCc2c1cc1n(c2=O)Cc2c-1nc1cc(F)c(C)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@@H](C)O] 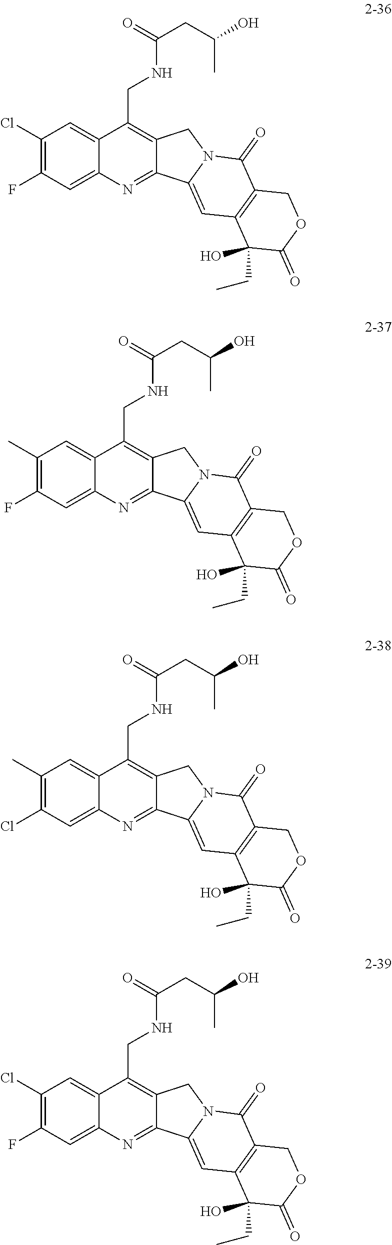 [SMILES: CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(F)c(C)cc1c2CNC(=O)C[C@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O] 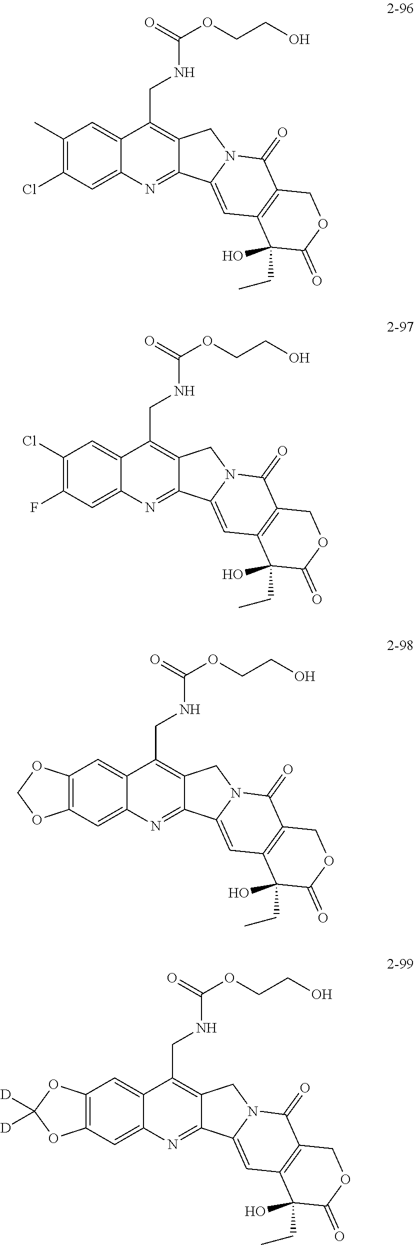 [SMILES: CC[C@@]1(O)C(=O)OCc2c1cc1n(c2=O)Cc2c-1nc1cc(Cl)c(C)cc1c2CNC(=O)OCCO ; CC[C@@]1(O)C(=O)OCc2c1cc1n(c2=O)Cc2c-1nc1cc(F)c(Cl)cc1c2CNC(=O)OCCO ; CC[C@@]1(O)C(=O)OCc2c1cc1n(c2=O)Cc2c-1nc1cc3c(cc1c2CNC(=O)OCCO)OCO3 ; [2H]C1([2H])Oc2cc3nc4c(c(CNC(=O)OCCO)c3cc2O1)Cn1c-4cc2c(c1=O)COC(=O)[C@]2(O)CC] 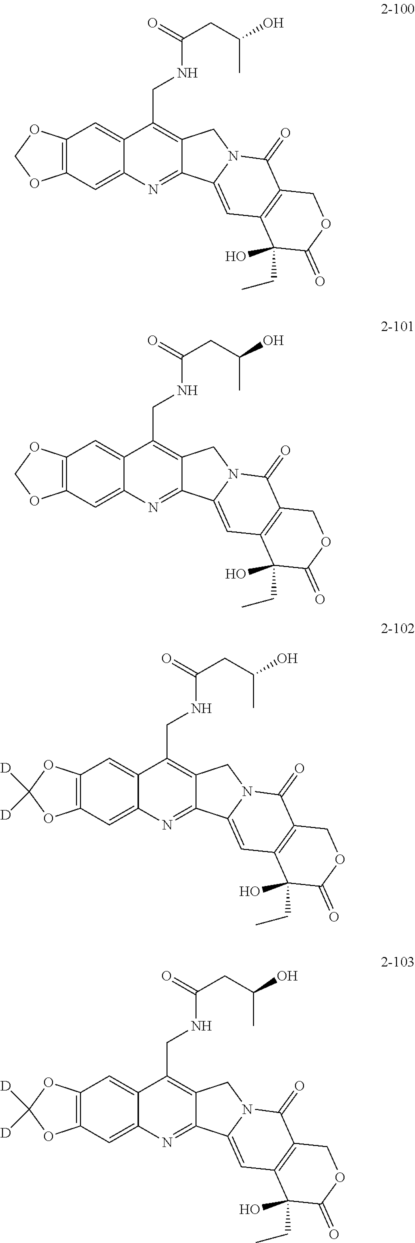 [SMILES: CC[C@@]1(O)C(=O)OCc2c1cc1n(c2=O)Cc2c-1nc1cc3c(cc1c2CNC(=O)C[C@@H](C)O)OCO3 ; CC[C@@]1(O)C(=O)OCc2c1cc1n(c2=O)Cc2c-1nc1cc3c(cc1c2CNC(=O)C[C@H](C)O)OCO3 ; [2H]C1([2H])Oc2cc3nc4c(c(CNC(=O)C[C@@H](C)O)c3cc2O1)Cn1c-4cc2c(c1=O)COC(=O)[C@]2(O)CC ; [2H]C1([2H])Oc2cc3nc4c(c(CNC(=O)C[C@H](C)O)c3cc2O1)Cn1c-4cc2c(c1=O)COC(=O)[C@]2(O)CC] 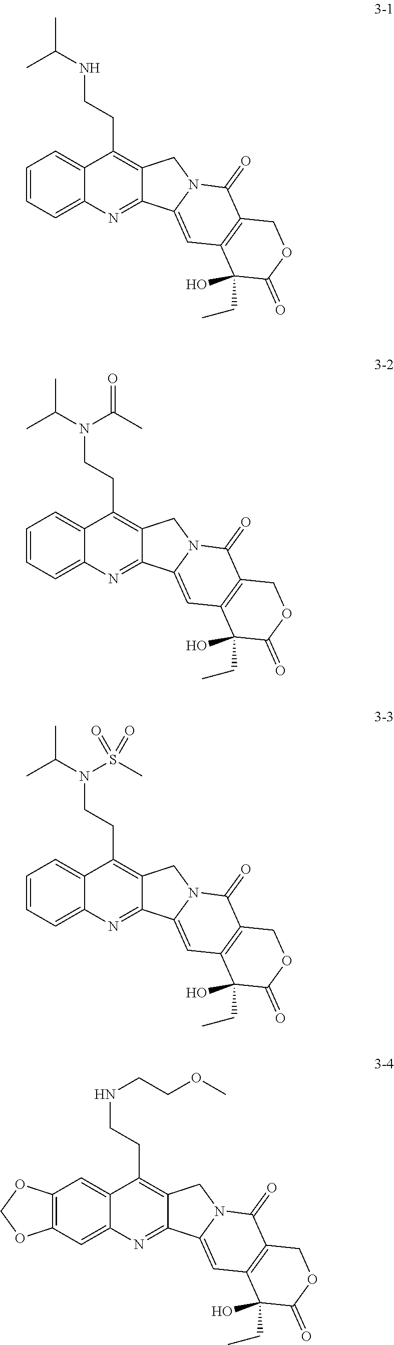 [SMILES: CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1cc3c(cc1c2CCNCCOC)OCO3] 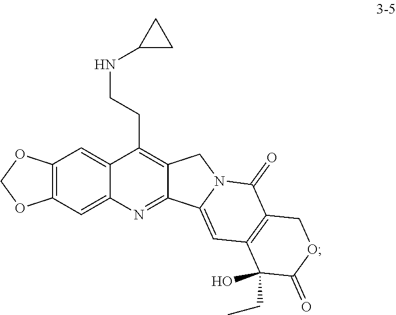 [SMILES: CC[C@@]1(O)C(=O)OCc2c1cc1n(c2=O)Cc2c-1nc1cc3c(cc1c2CCNC1CC1)OCO3]
or, the cytotoxic drug is selected from the group consisting of the following compounds or isotopically labeled compounds thereof: 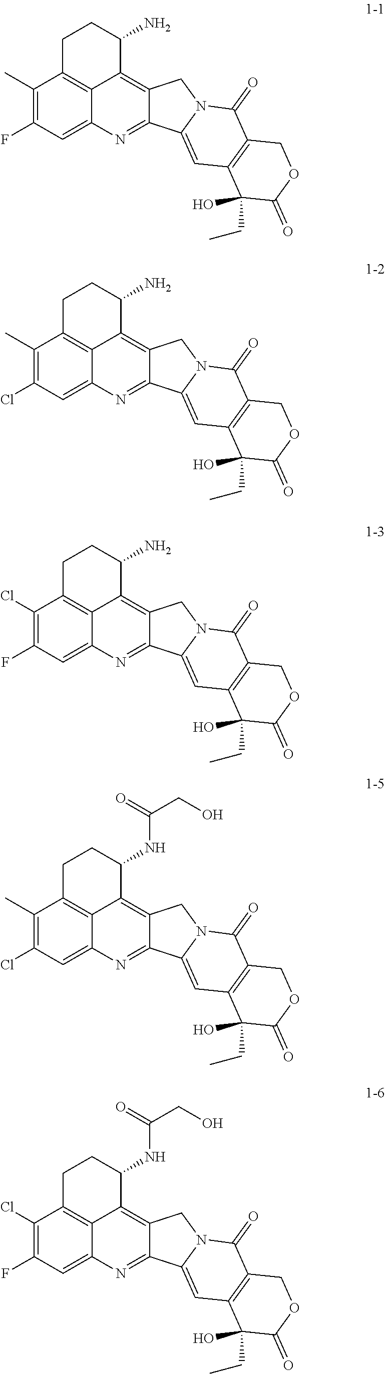 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3] 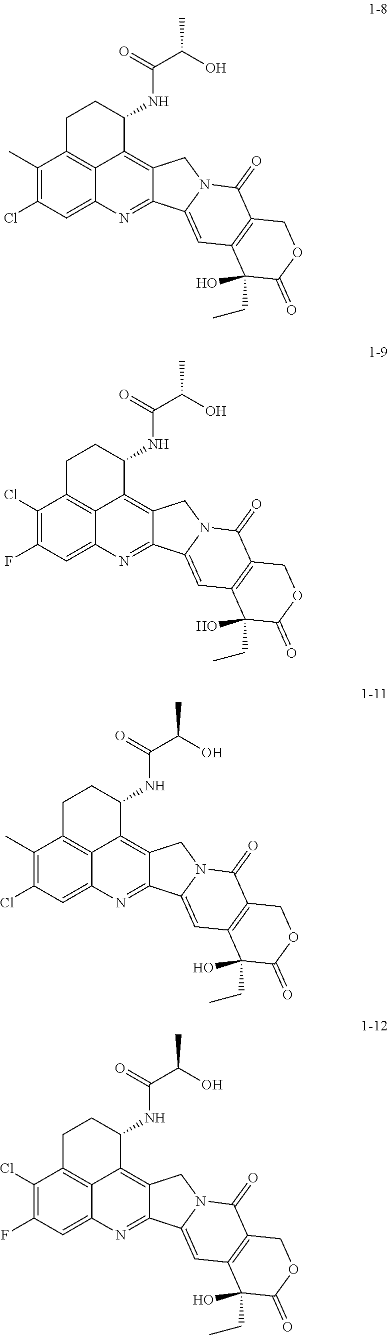 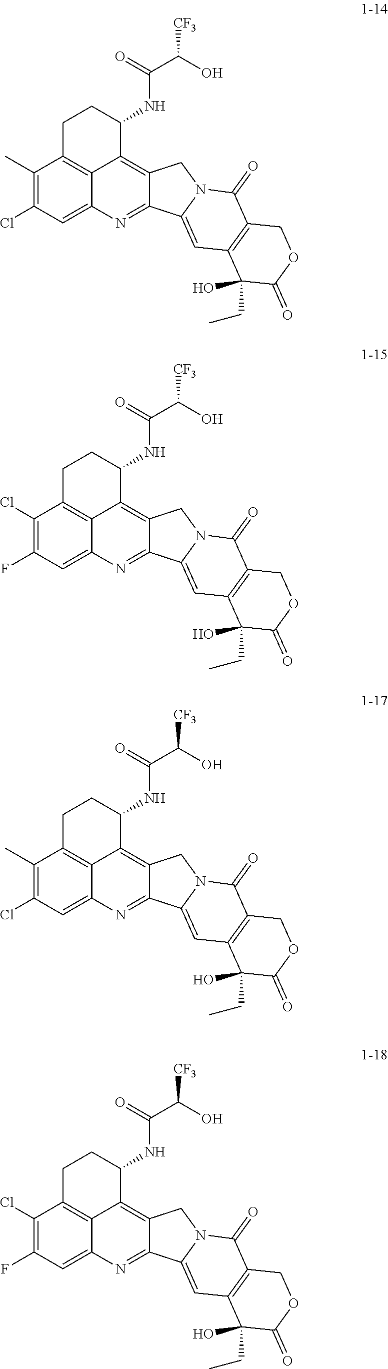 [SMILES: CC[C@@]1(O)C(=O)OCc2c1cc1n(c2=O)Cc2c-1nc1cc(Cl)c(C)c3c1c2[C@@H](NC(=O)[C@@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@@H](O)C(F)(F)F)CC3 ; CC[C@@]1(O)C(=O)OCc2c1cc1n(c2=O)Cc2c-1nc1cc(Cl)c(C)c3c1c2[C@@H](NC(=O)[C@H](O)C(F)(F)F)CC3 ; CC[C@@]1(O)C(=O)OCc2c1cc1n(c2=O)Cc2c-1nc1cc(F)c(Cl)c3c1c2[C@@H](NC(=O)[C@H](O)C(F)(F)F)CC3] 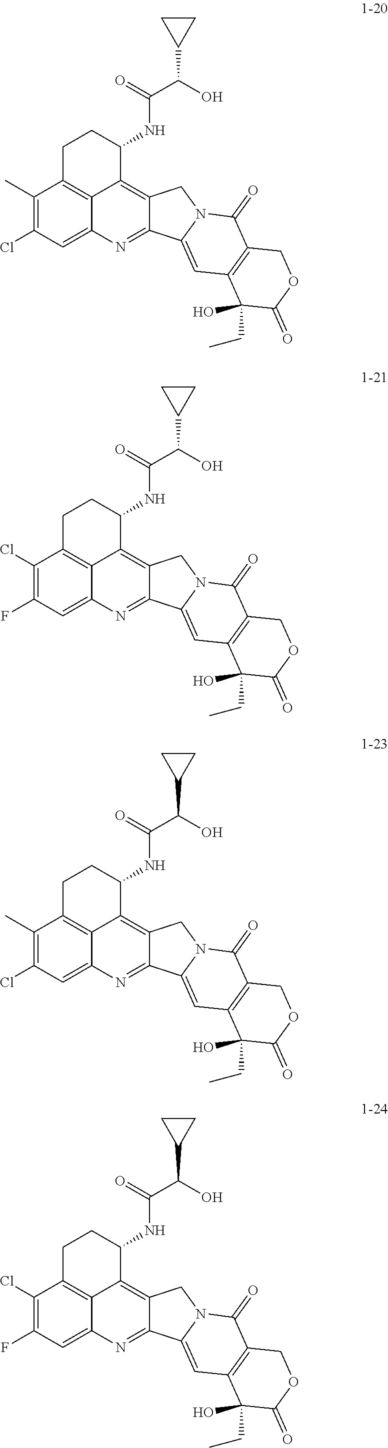 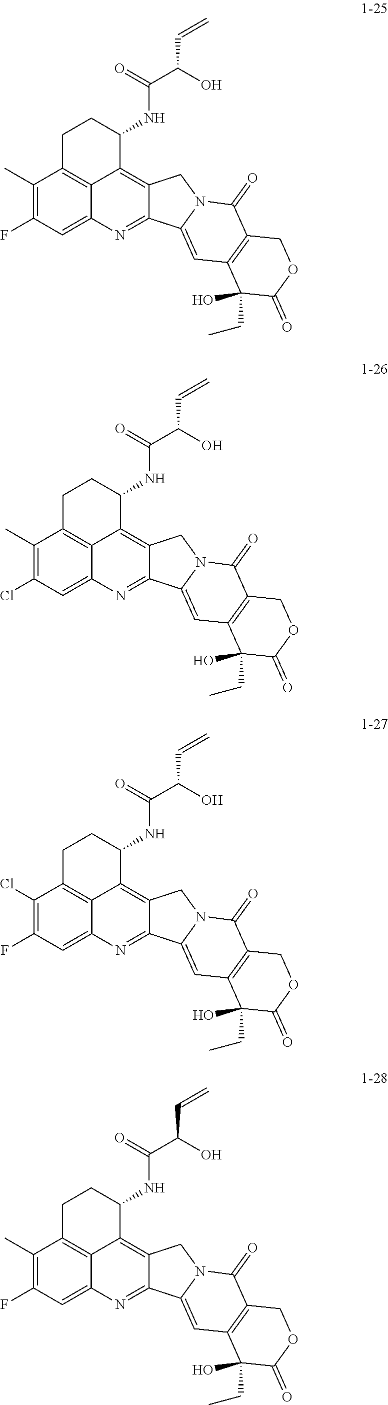 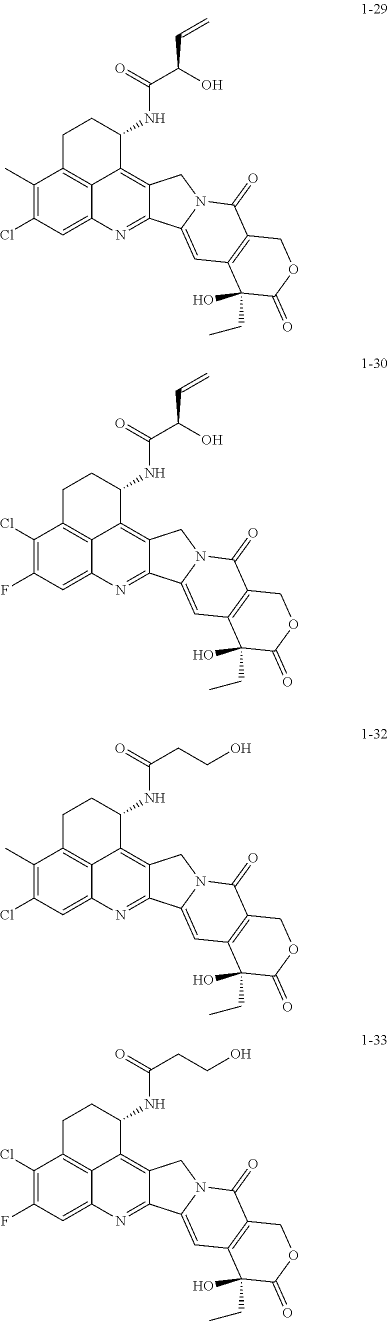 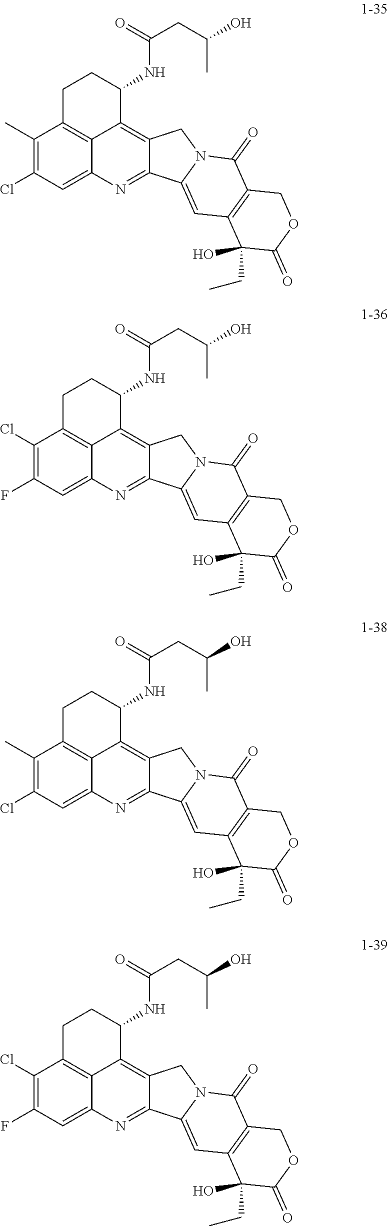 [SMILES: CC[C@@]1(O)C(=O)OCc2c1cc1n(c2=O)Cc2c-1nc1cc(Cl)c(C)c3c1c2[C@@H](NC(=O)C[C@@H](C)O)CC3 ; CC[C@@]1(O)C(=O)OCc2c1cc1n(c2=O)Cc2c-1nc1cc(F)c(Cl)c3c1c2[C@@H](NC(=O)C[C@@H](C)O)CC3 ; CC[C@@]1(O)C(=O)OCc2c1cc1n(c2=O)Cc2c-1nc1cc(Cl)c(C)c3c1c2[C@@H](NC(=O)C[C@H](C)O)CC3 ; CC[C@@]1(O)C(=O)OCc2c1cc1n(c2=O)Cc2c-1nc1cc(F)c(Cl)c3c1c2[C@@H](NC(=O)C[C@H](C)O)CC3] 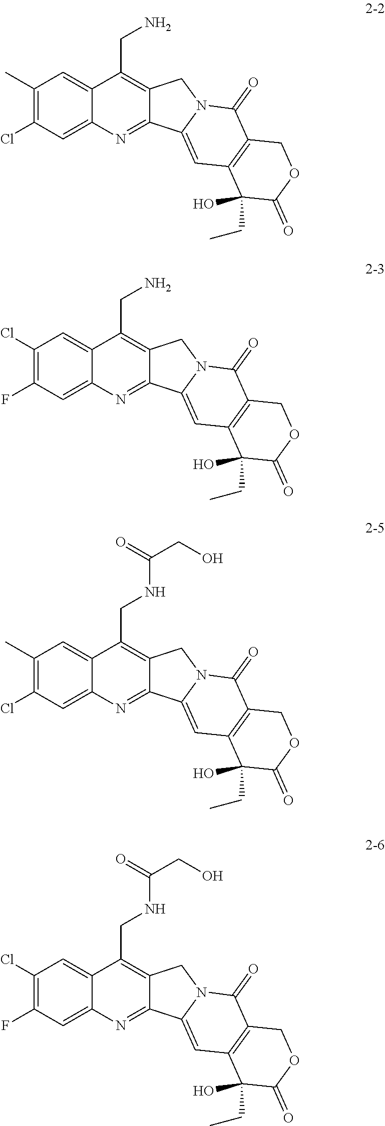 [SMILES: CC[C@@]1(O)C(=O)OCc2c1cc1n(c2=O)Cc2c-1nc1cc(Cl)c(C)cc1c2CN ; CC[C@@]1(O)C(=O)OCc2c1cc1n(c2=O)Cc2c-1nc1cc(F)c(Cl)cc1c2CN ; CC[C@@]1(O)C(=O)OCc2c1cc1n(c2=O)Cc2c-1nc1cc(Cl)c(C)cc1c2CNC(=O)CO ; CC[C@@]1(O)C(=O)OCc2c1cc1n(c2=O)Cc2c-1nc1cc(F)c(Cl)cc1c2CNC(=O)CO] 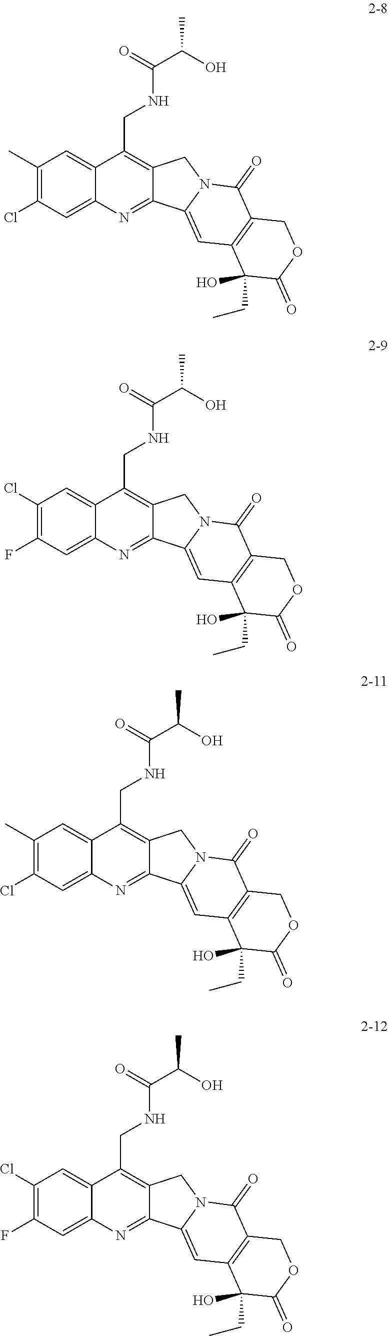 [SMILES: CC[C@@]1(O)C(=O)OCc2c1cc1n(c2=O)Cc2c-1nc1cc(Cl)c(C)cc1c2CNC(=O)[C@H](C)O ; CC[C@@]1(O)C(=O)OCc2c1cc1n(c2=O)Cc2c-1nc1cc(F)c(Cl)cc1c2CNC(=O)[C@H](C)O ; CC[C@@]1(O)C(=O)OCc2c1cc1n(c2=O)Cc2c-1nc1cc(Cl)c(C)cc1c2CNC(=O)[C@@H](C)O ; CC[C@@]1(O)C(=O)OCc2c1cc1n(c2=O)Cc2c-1nc1cc(F)c(Cl)cc1c2CNC(=O)[C@@H](C)O] 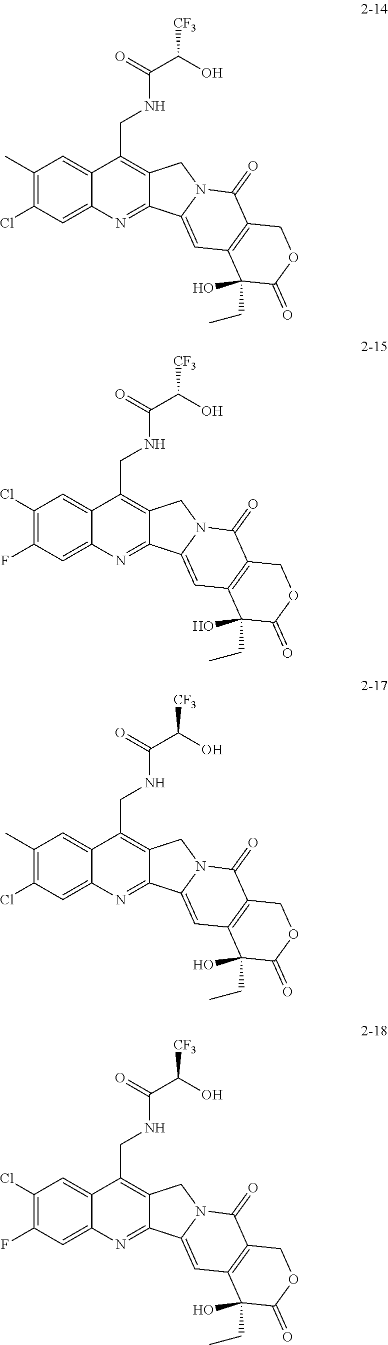 [SMILES: CC[C@@]1(O)C(=O)OCc2c1cc1n(c2=O)Cc2c-1nc1cc(Cl)c(C)cc1c2CNC(=O)[C@@H](O)C(F)(F)F ; CC[C@@]1(O)C(=O)OCc2c1cc1n(c2=O)Cc2c-1nc1cc(F)c(Cl)cc1c2CNC(=O)[C@@H](O)C(F)(F)F ; CC[C@@]1(O)C(=O)OCc2c1cc1n(c2=O)Cc2c-1nc1cc(Cl)c(C)cc1c2CNC(=O)[C@H](O)C(F)(F)F ; CC[C@@]1(O)C(=O)OCc2c1cc1n(c2=O)Cc2c-1nc1cc(F)c(Cl)cc1c2CNC(=O)[C@H](O)C(F)(F)F] 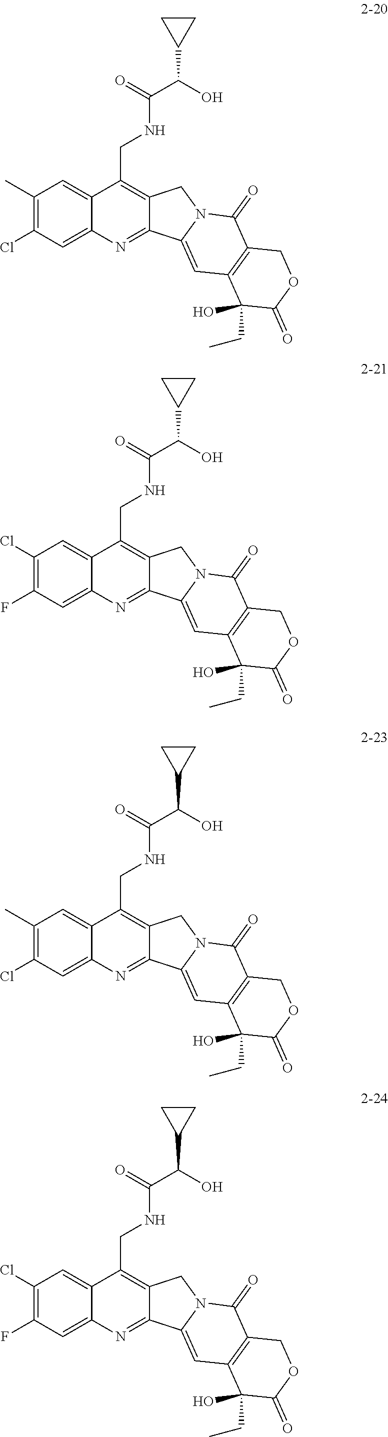 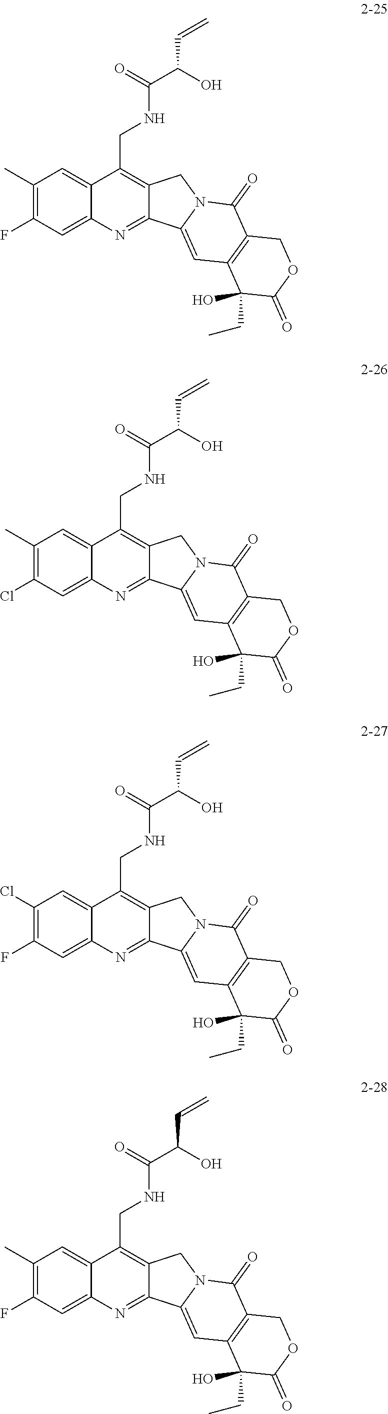 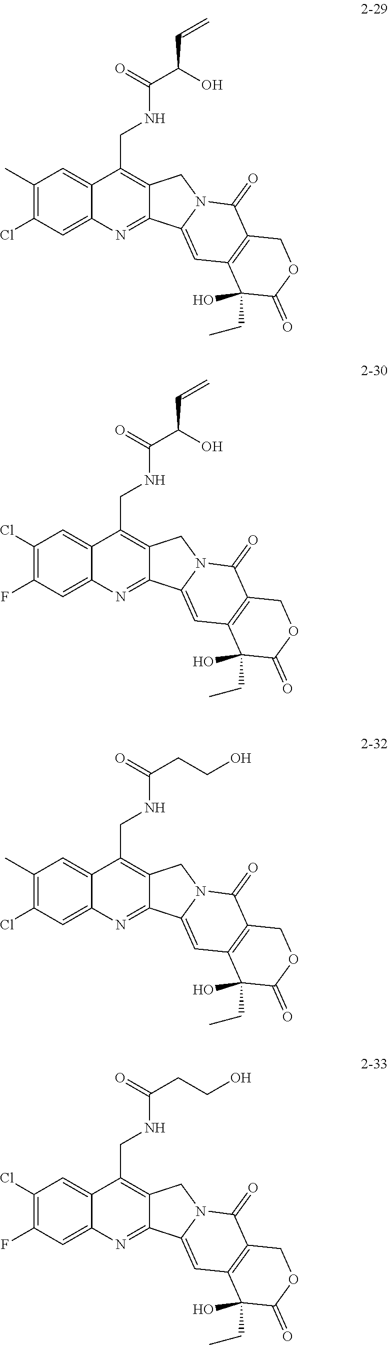 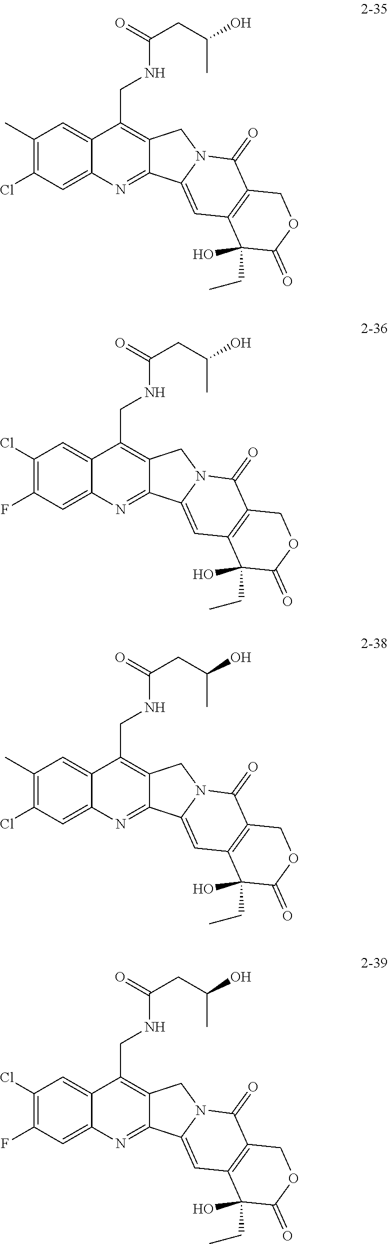 [SMILES: CC[C@@]1(O)C(=O)OCc2c1cc1n(c2=O)Cc2c-1nc1cc(Cl)c(C)cc1c2CNC(=O)C[C@@H](C)O ; CC[C@@]1(O)C(=O)OCc2c1cc1n(c2=O)Cc2c-1nc1cc(F)c(Cl)cc1c2CNC(=O)C[C@@H](C)O ; CC[C@@]1(O)C(=O)OCc2c1cc1n(c2=O)Cc2c-1nc1cc(Cl)c(C)cc1c2CNC(=O)C[C@H](C)O ; CC[C@@]1(O)C(=O)OCc2c1cc1n(c2=O)Cc2c-1nc1cc(F)c(Cl)cc1c2CNC(=O)C[C@H](C)O] 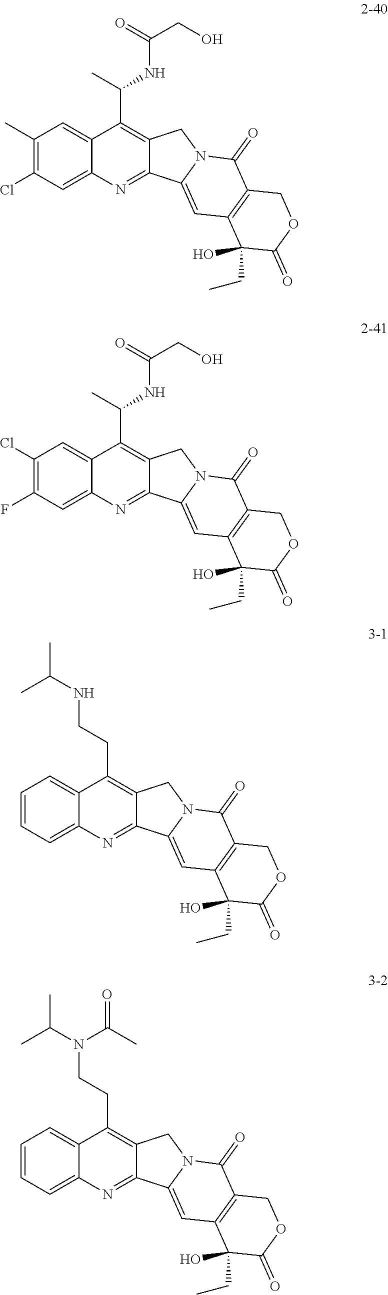 [SMILES: CC[C@@]1(O)C(=O)OCc2c1cc1n(c2=O)Cc2c-1nc1cc(Cl)c(C)cc1c2[C@H](C)NC(=O)CO ; CC[C@@]1(O)C(=O)OCc2c1cc1n(c2=O)Cc2c-1nc1cc(F)c(Cl)cc1c2[C@H](C)NC(=O)CO ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1ccccc1c2CCN(C(C)=O)C(C)C] 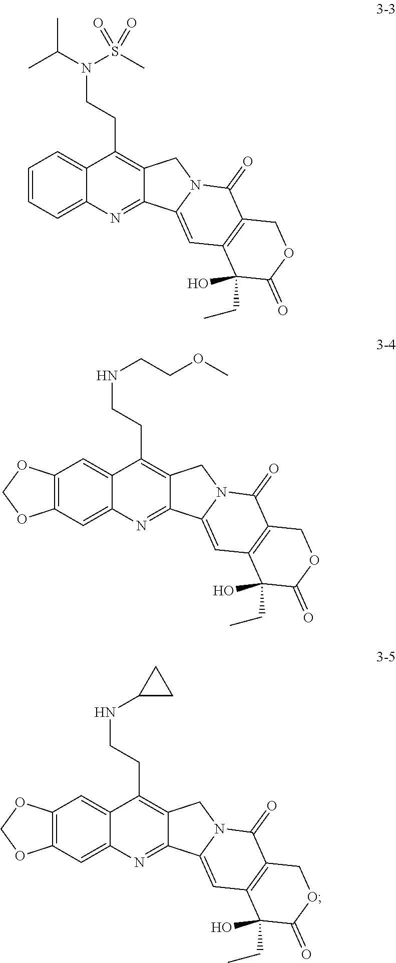
preferably, the cytotoxic drug is connected through-OH, primary amino, secondary amino or tertiary amino thereon to E in the compound.

7. The antibody-drug conjugate according to claim 1, wherein-M-L-E-D can be formed from the following structures: E′-1 to E′-50, F′-1 to F′-3, F′-7 to F′-12, G′-1 to G′-4, I′-10 to I′-49, E-1 to E-50, F-1 to F-14, G-1 to G-26, I-1 to I-50, for example, via a substitution reaction (e.g., through the removal of the methylsulfonyl group thereon): 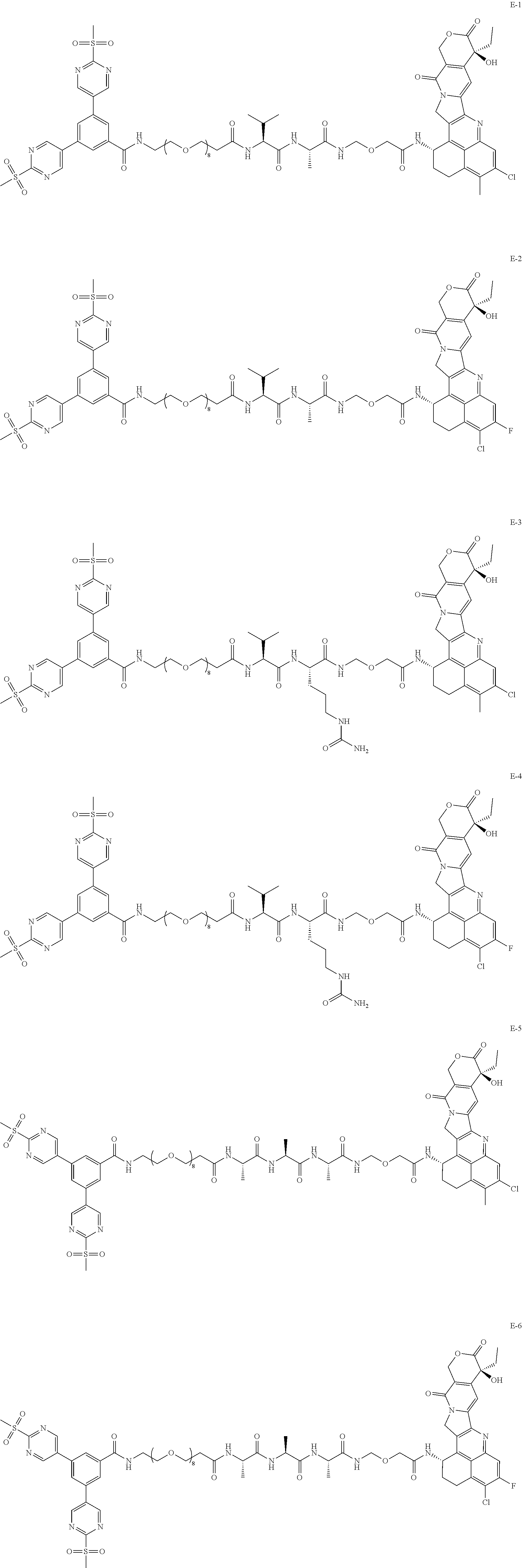 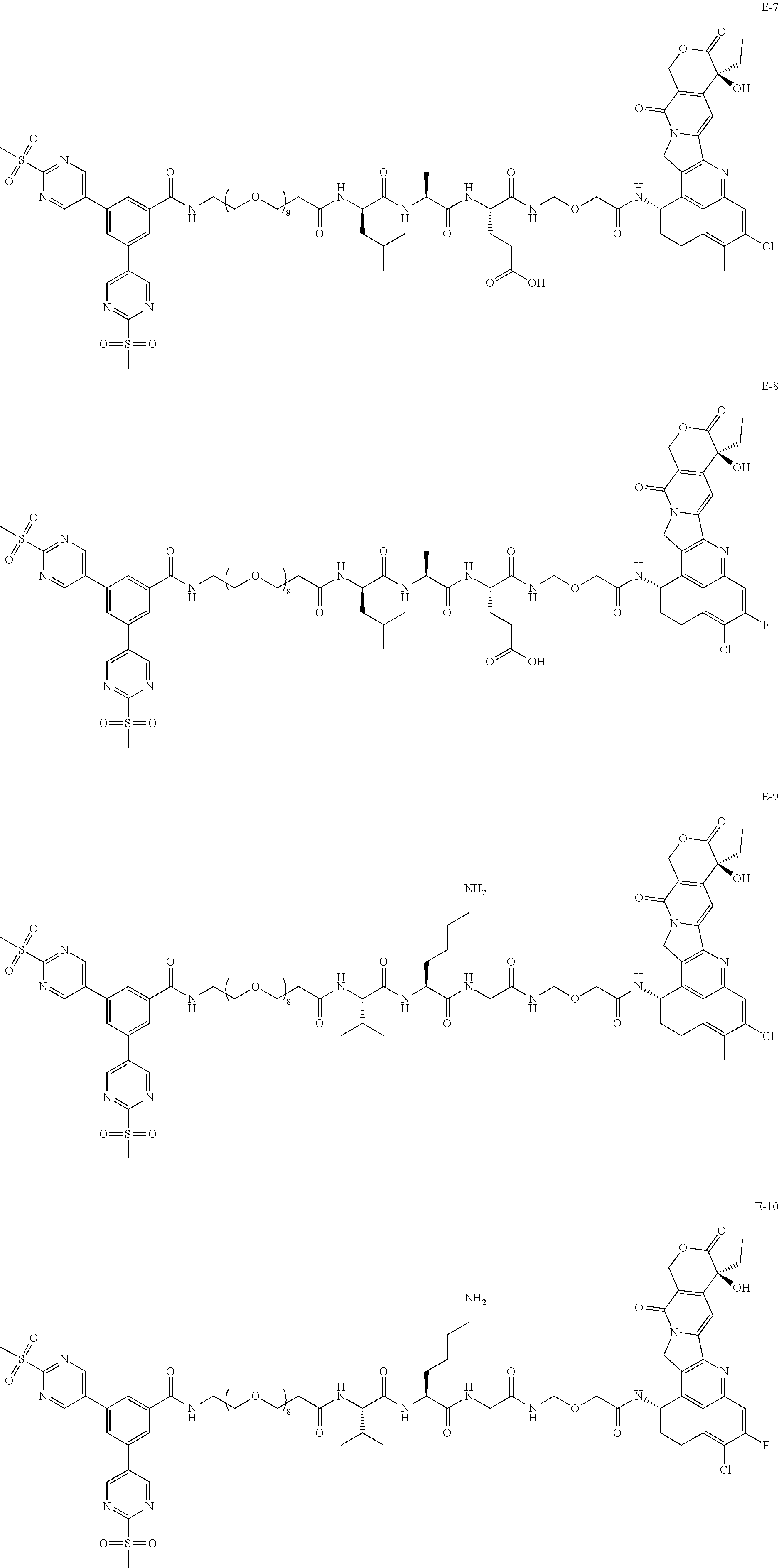 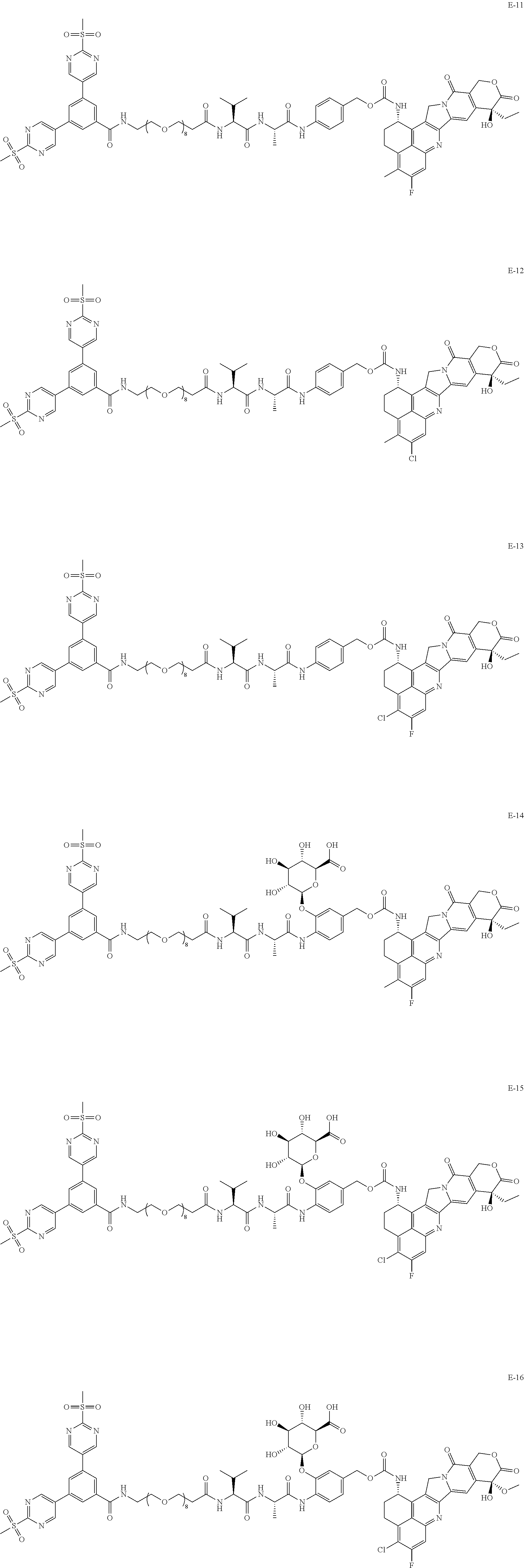 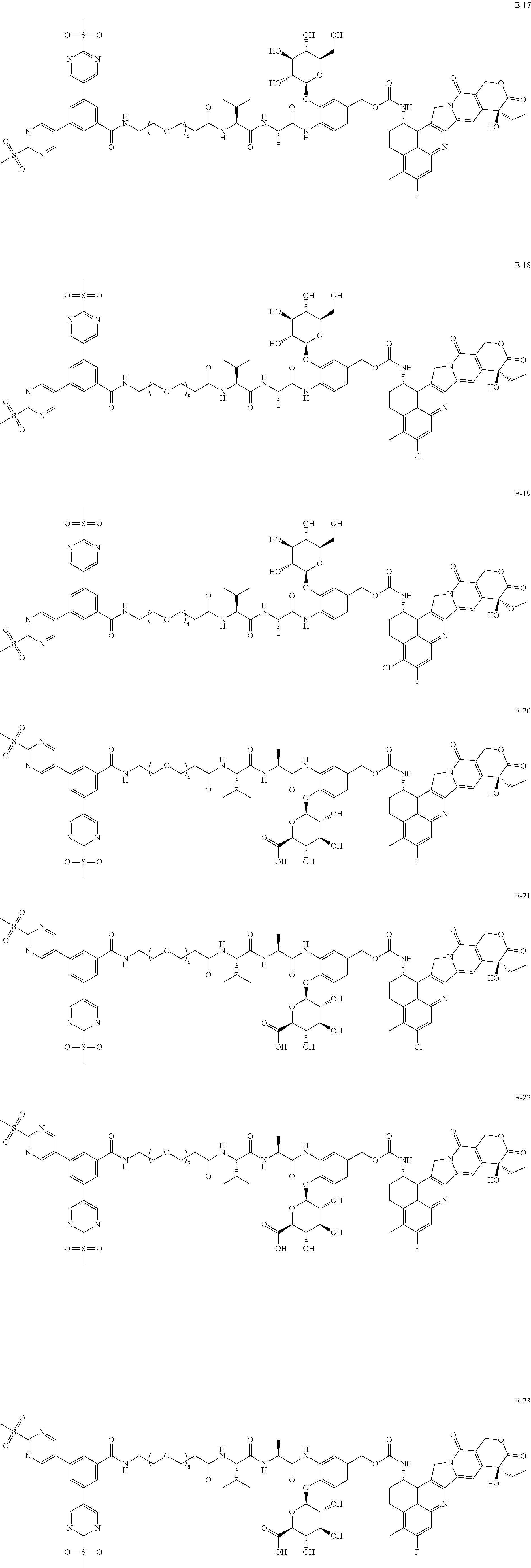 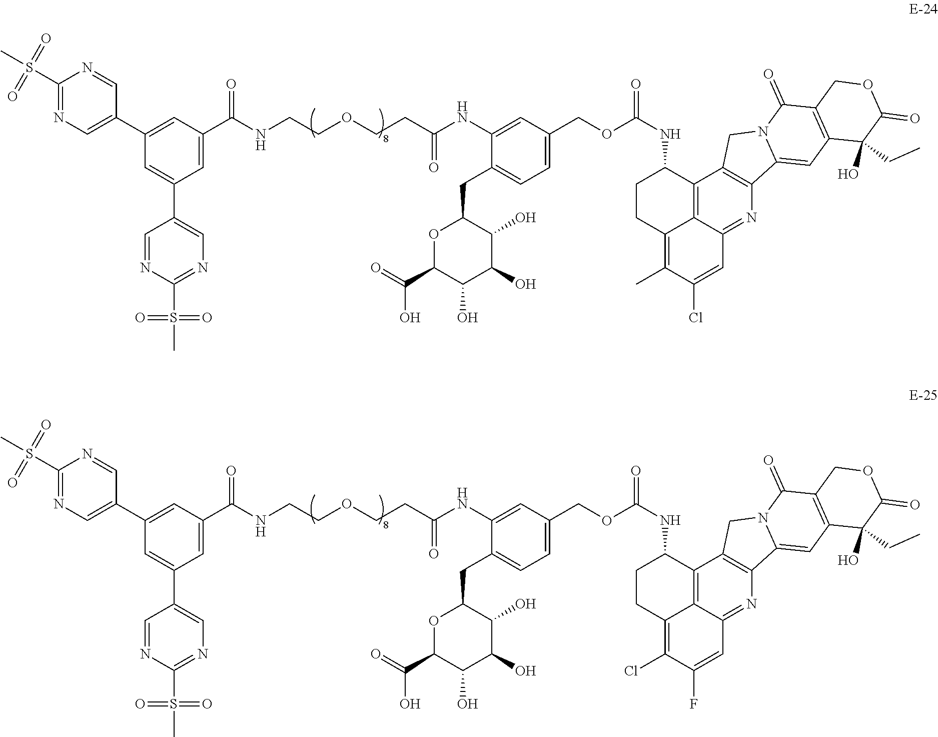 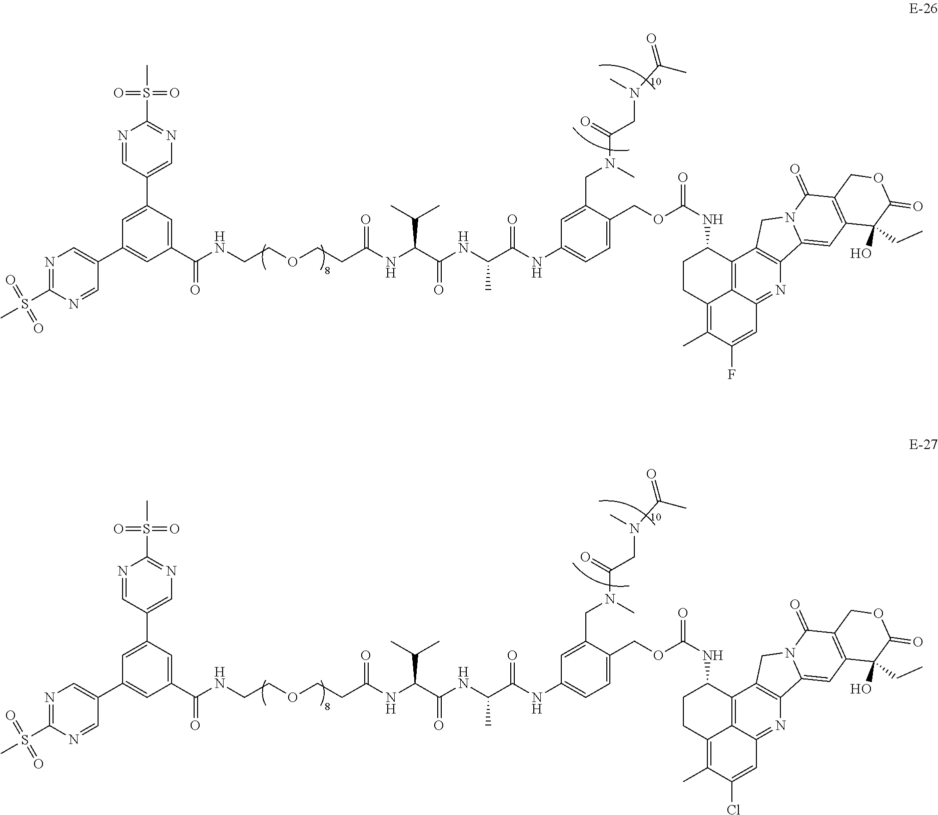 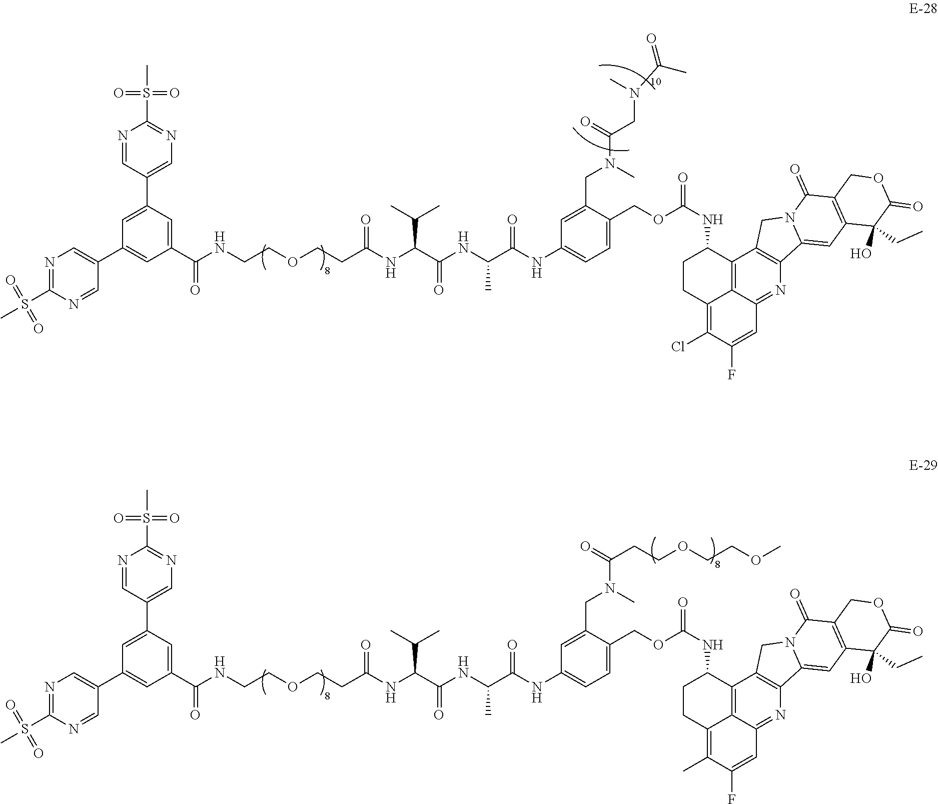 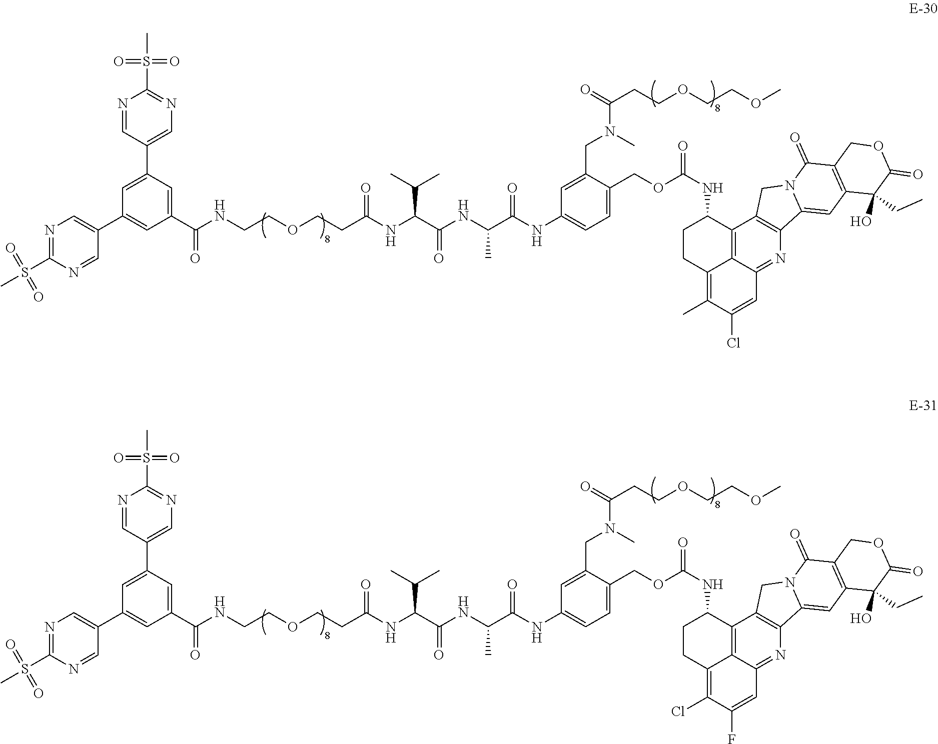 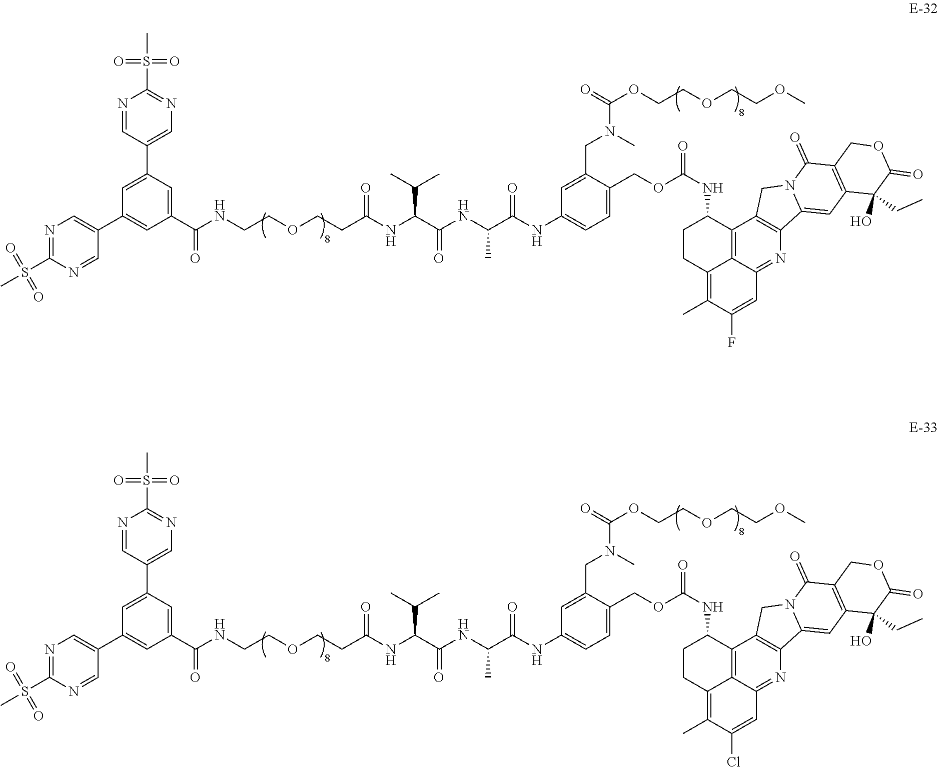 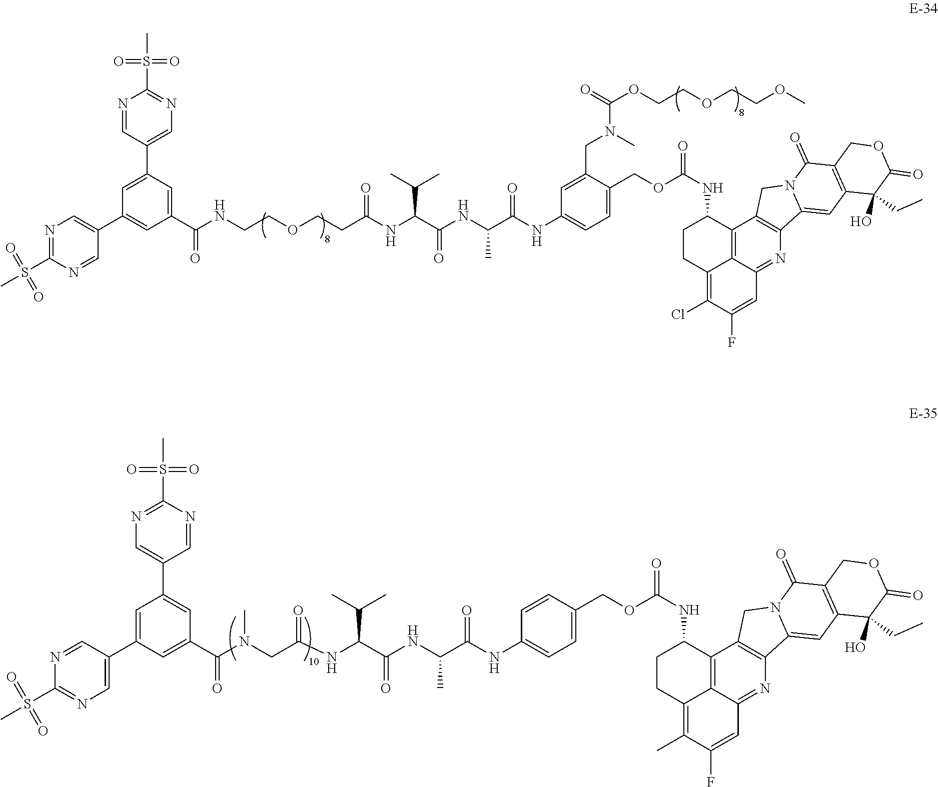 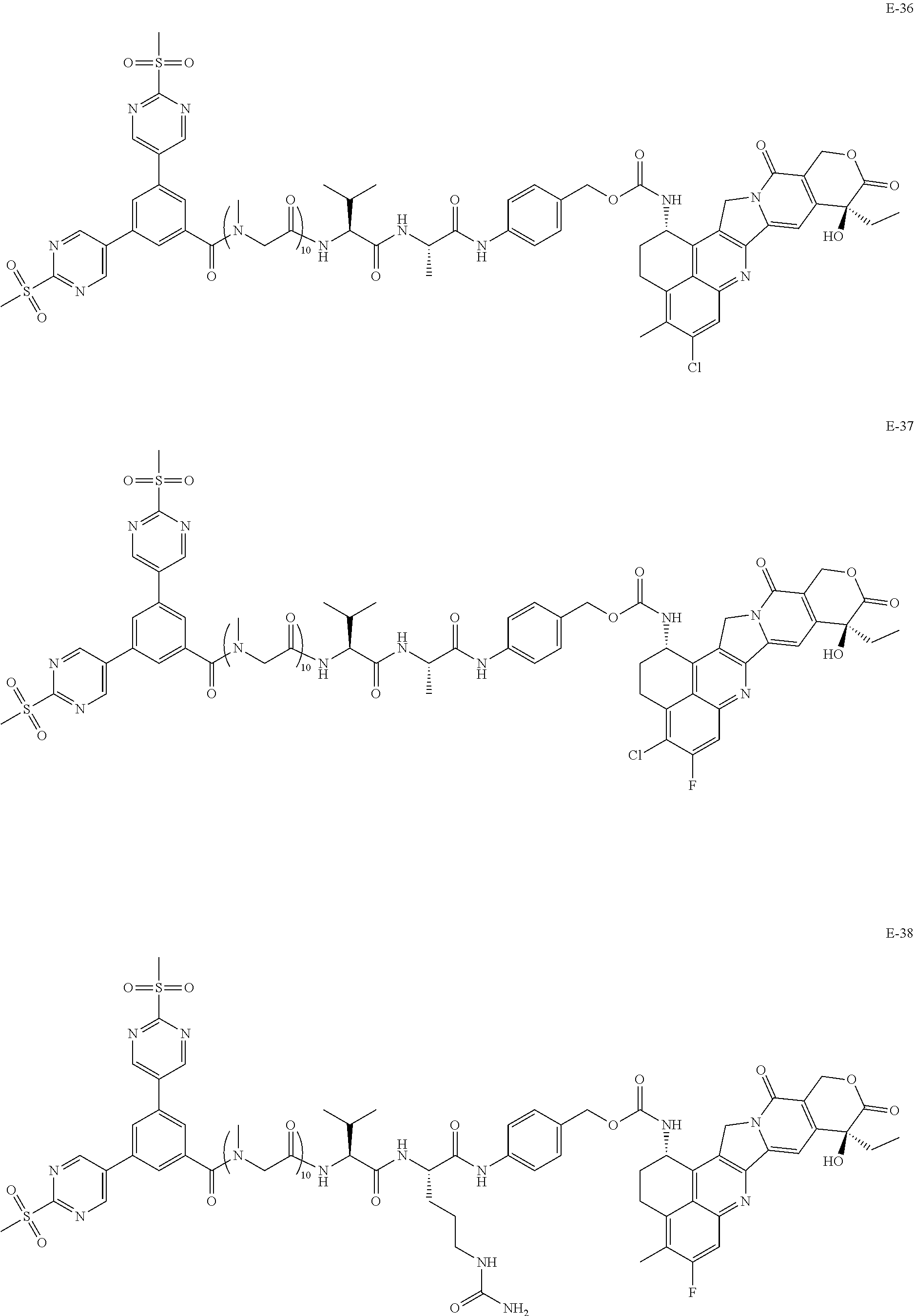 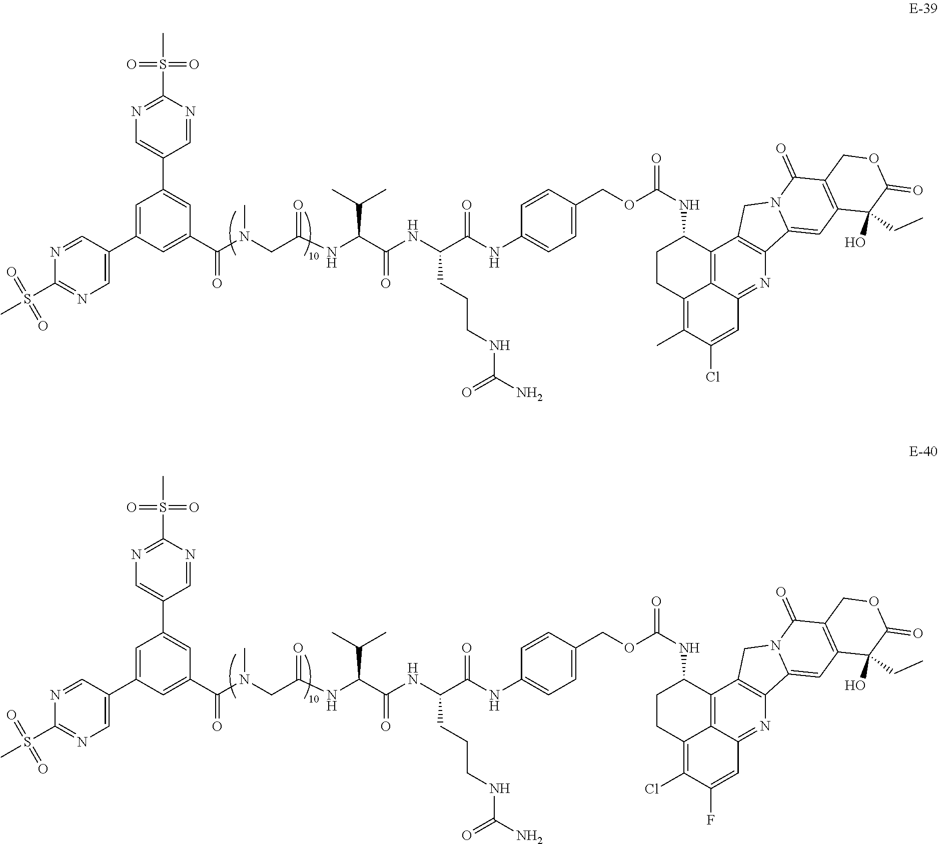 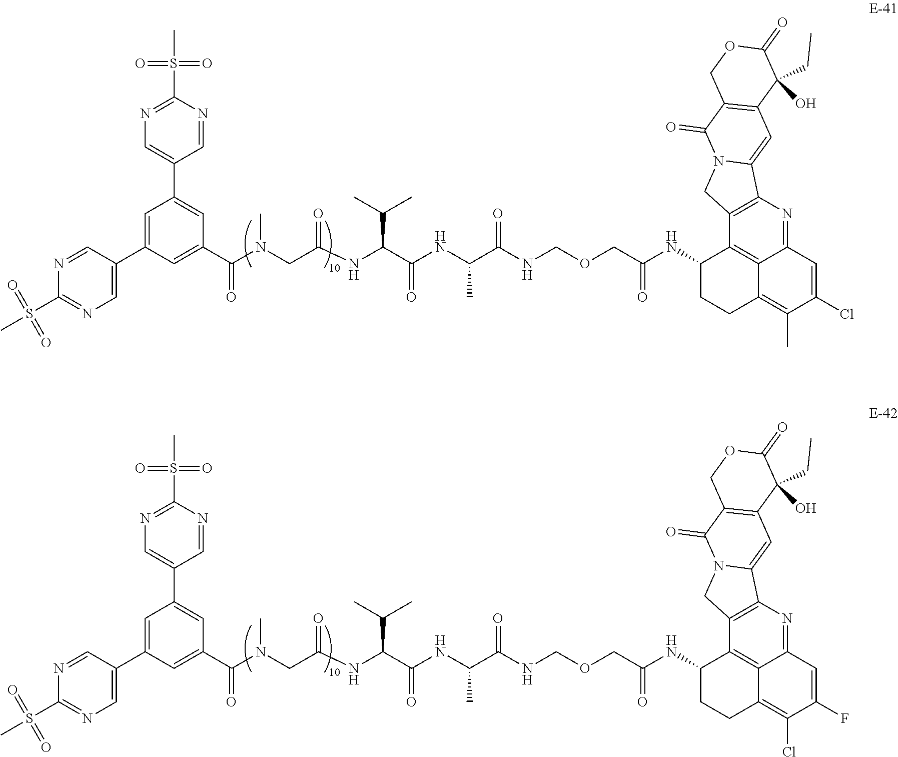 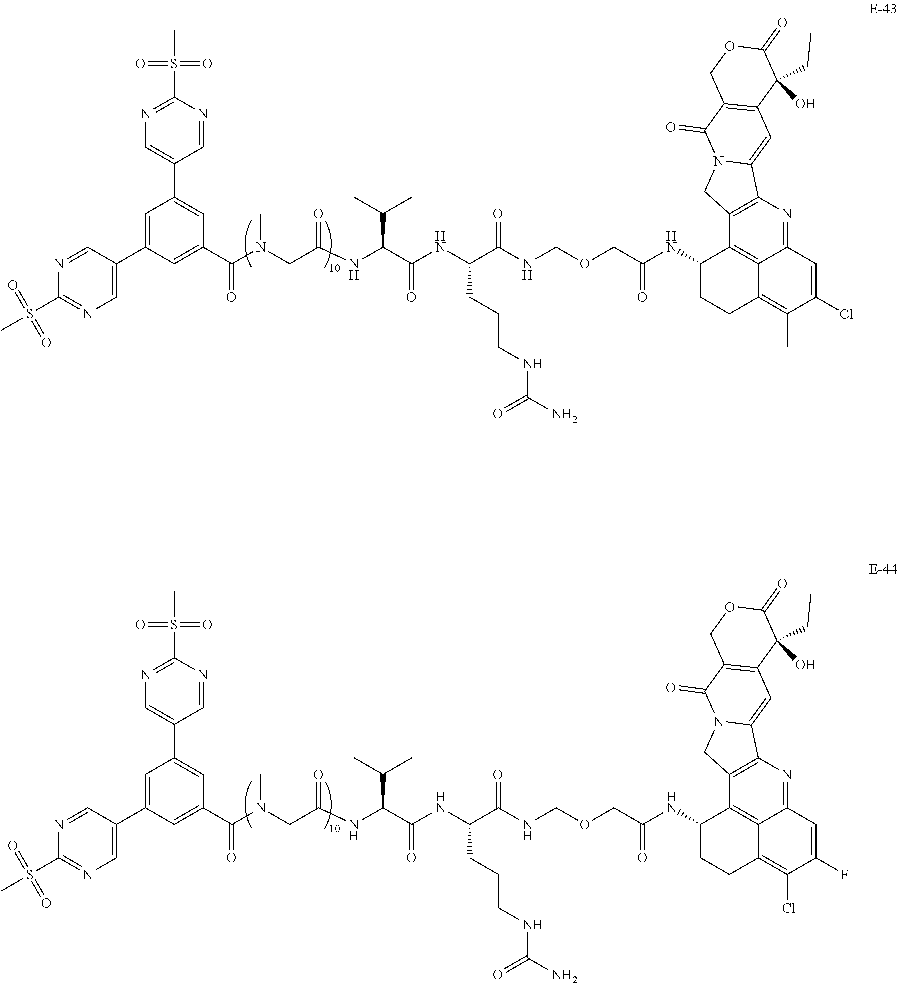 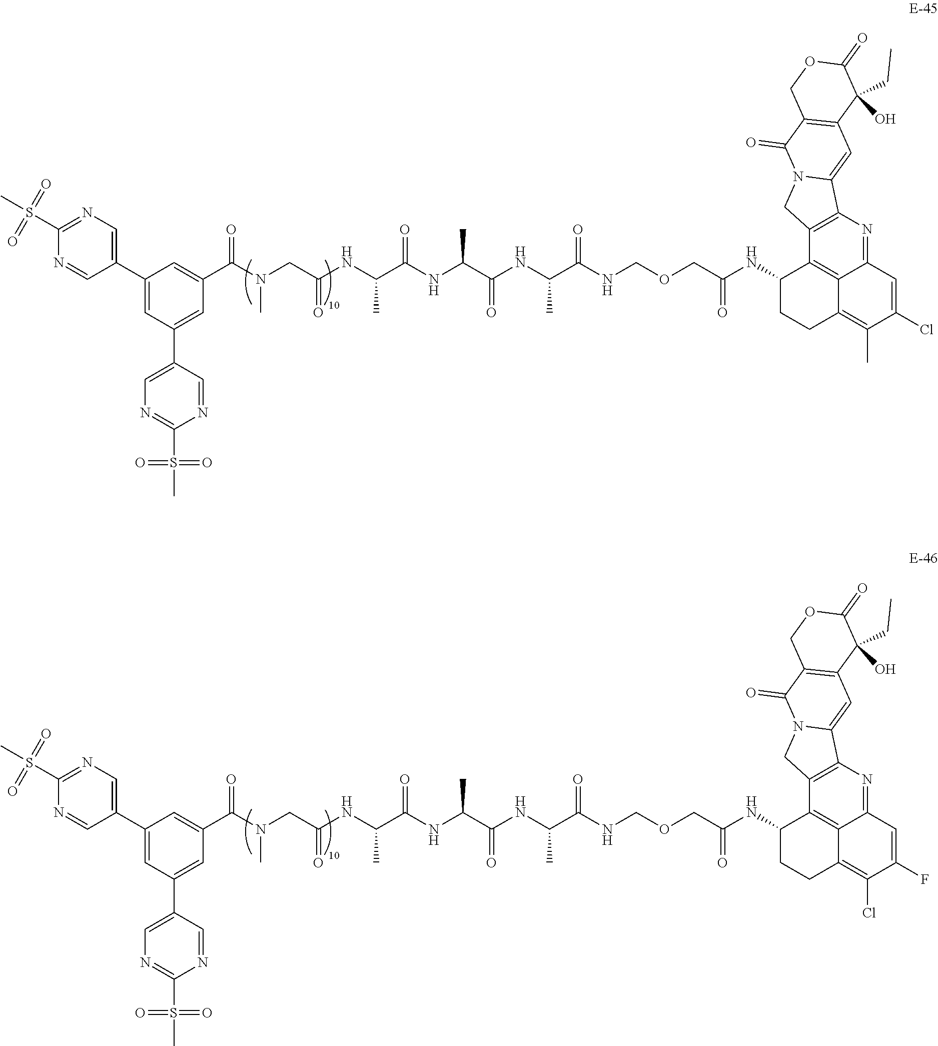 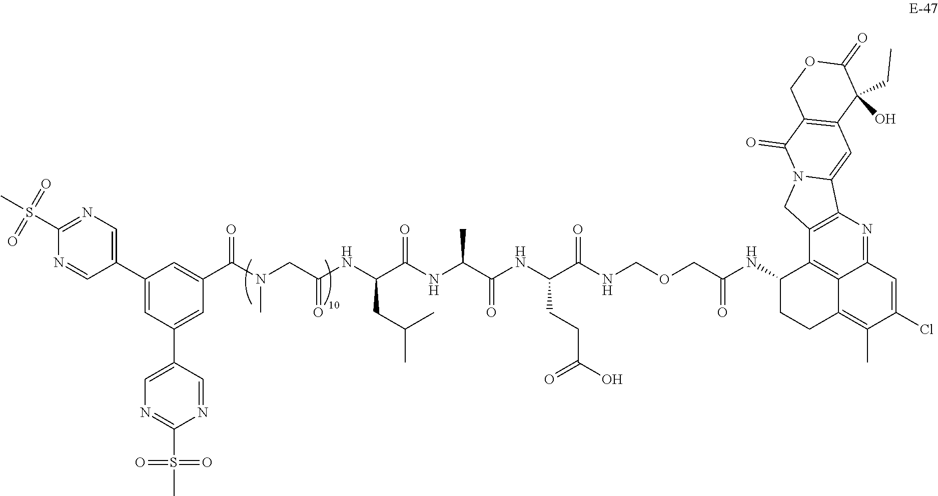 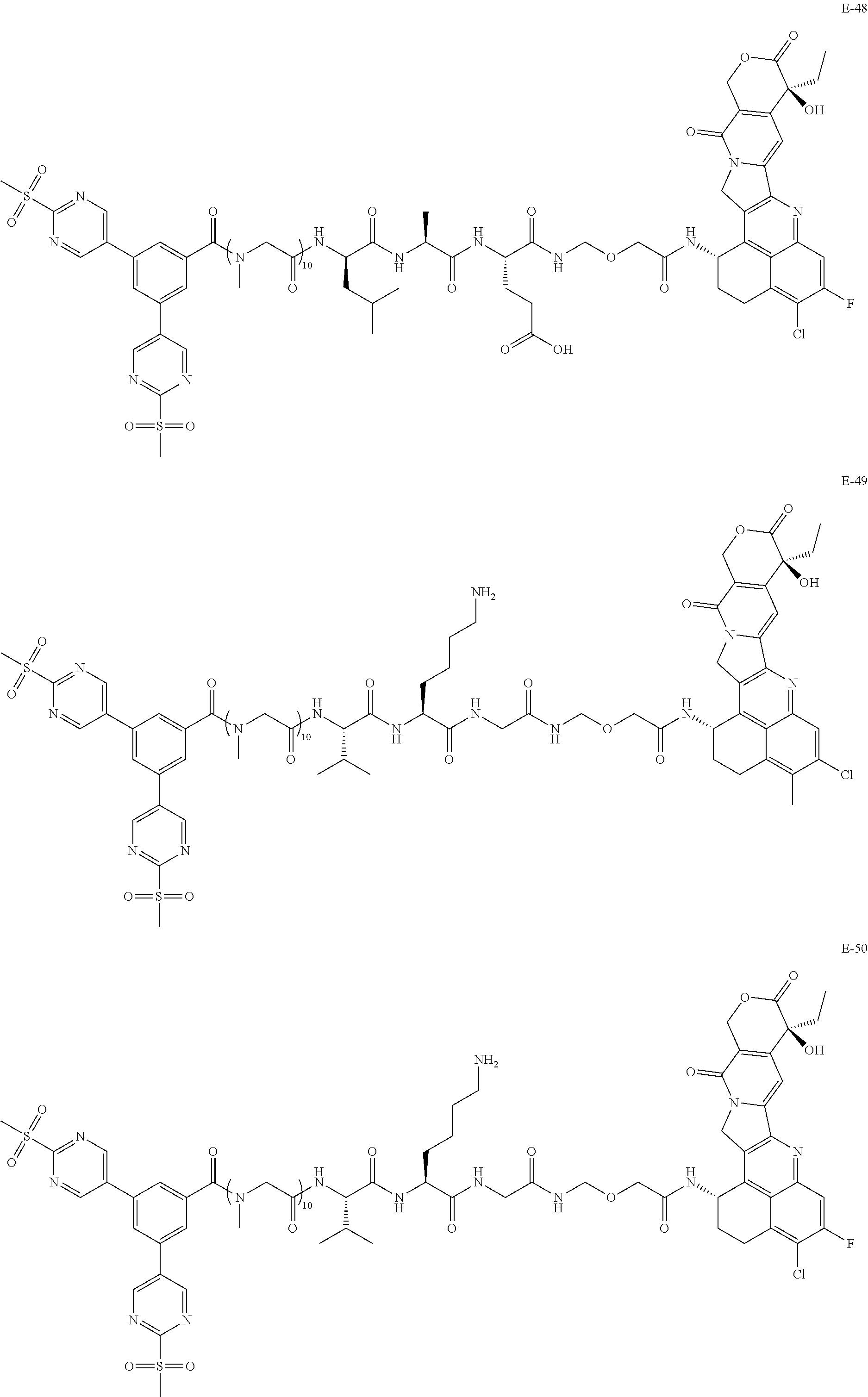 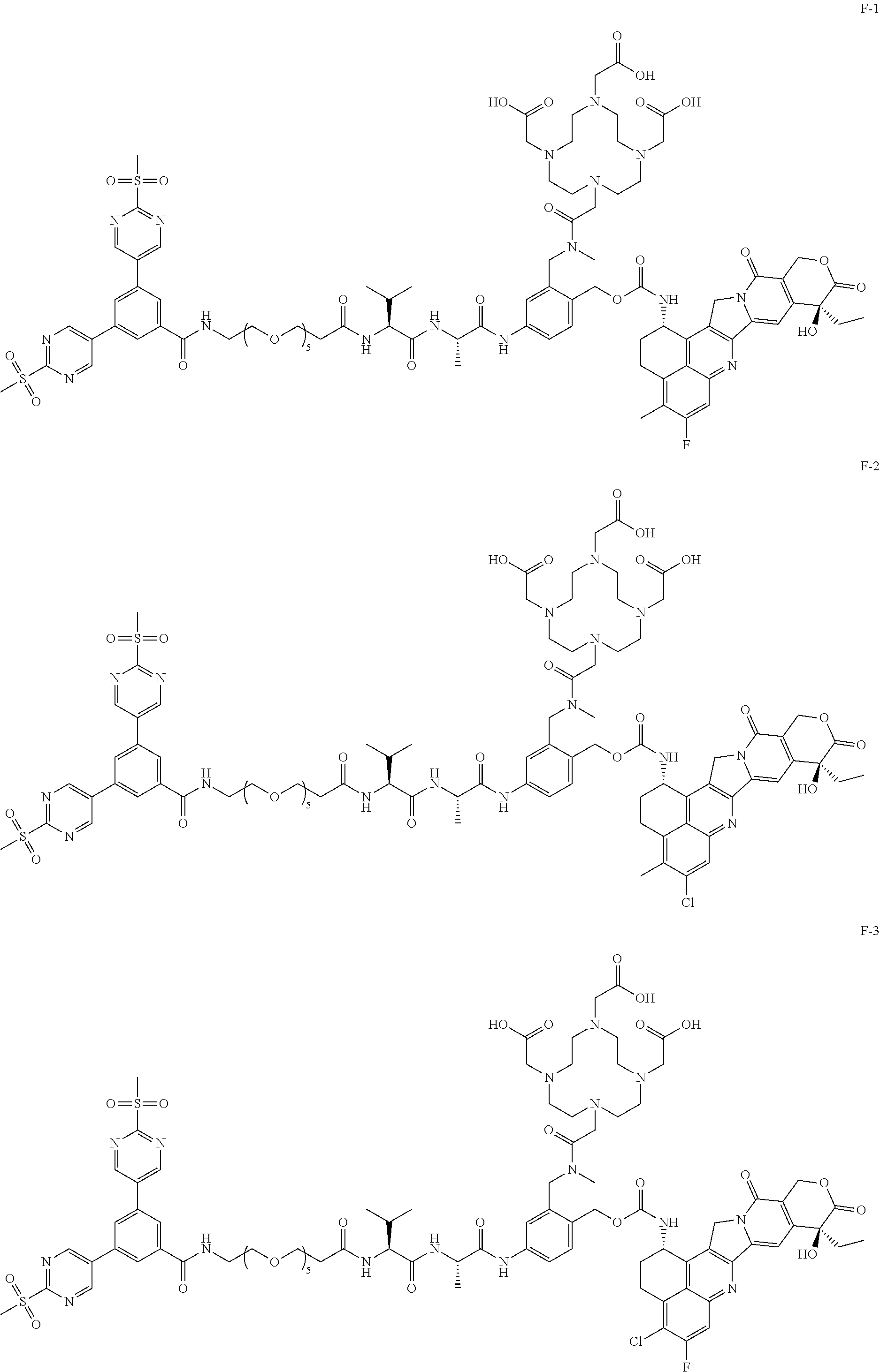 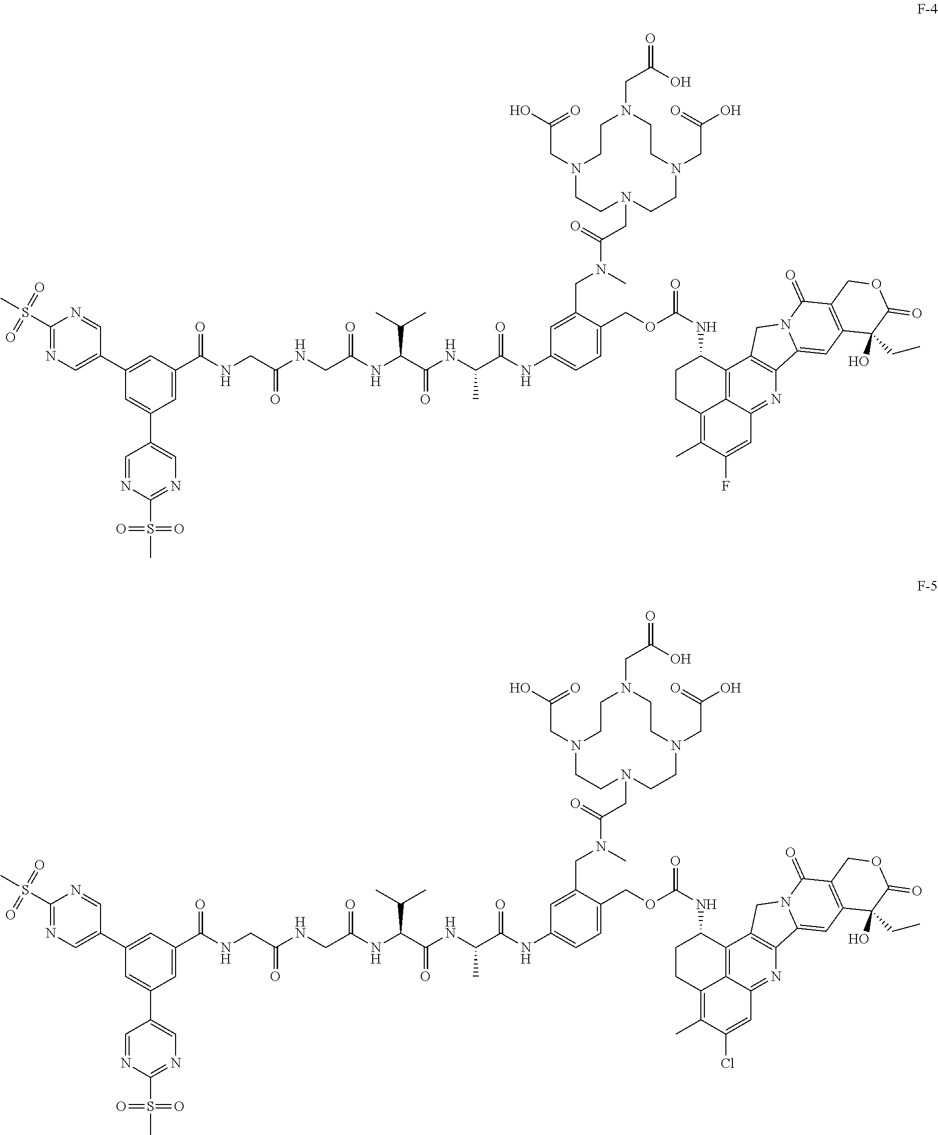 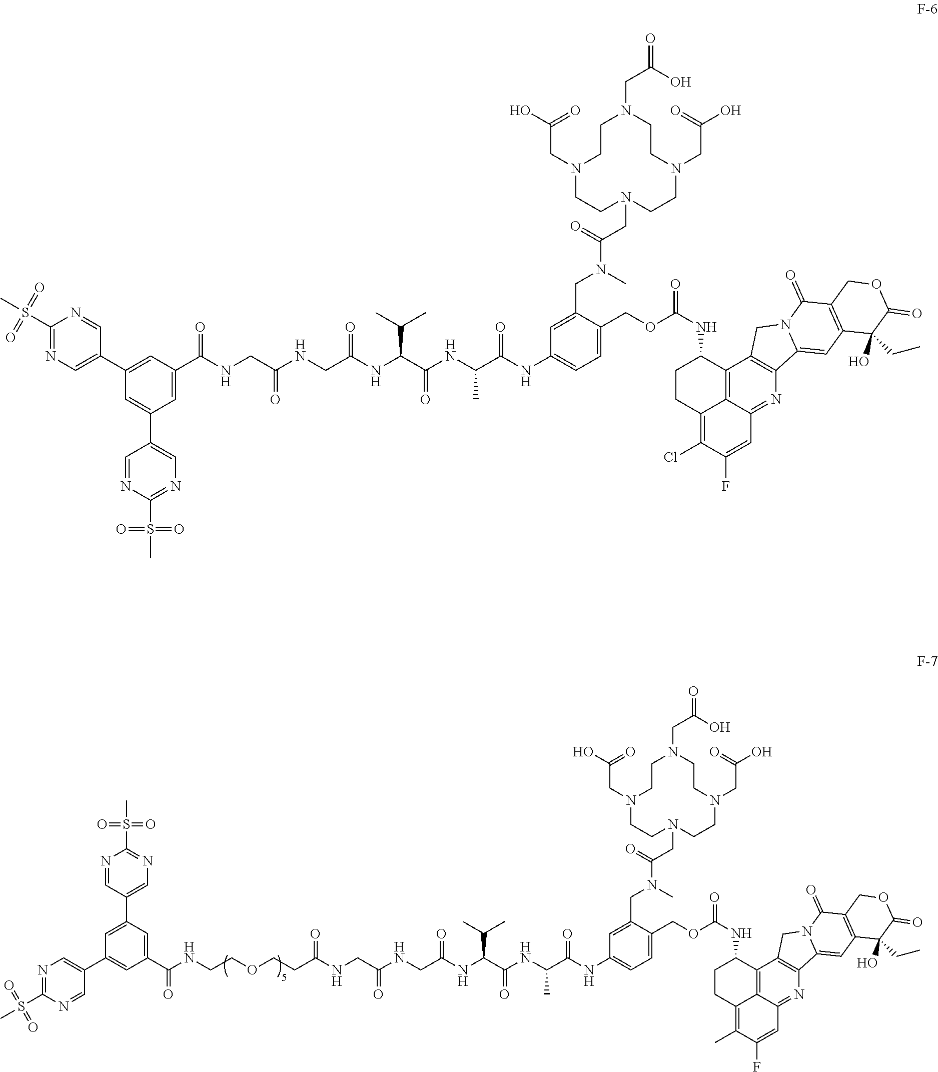 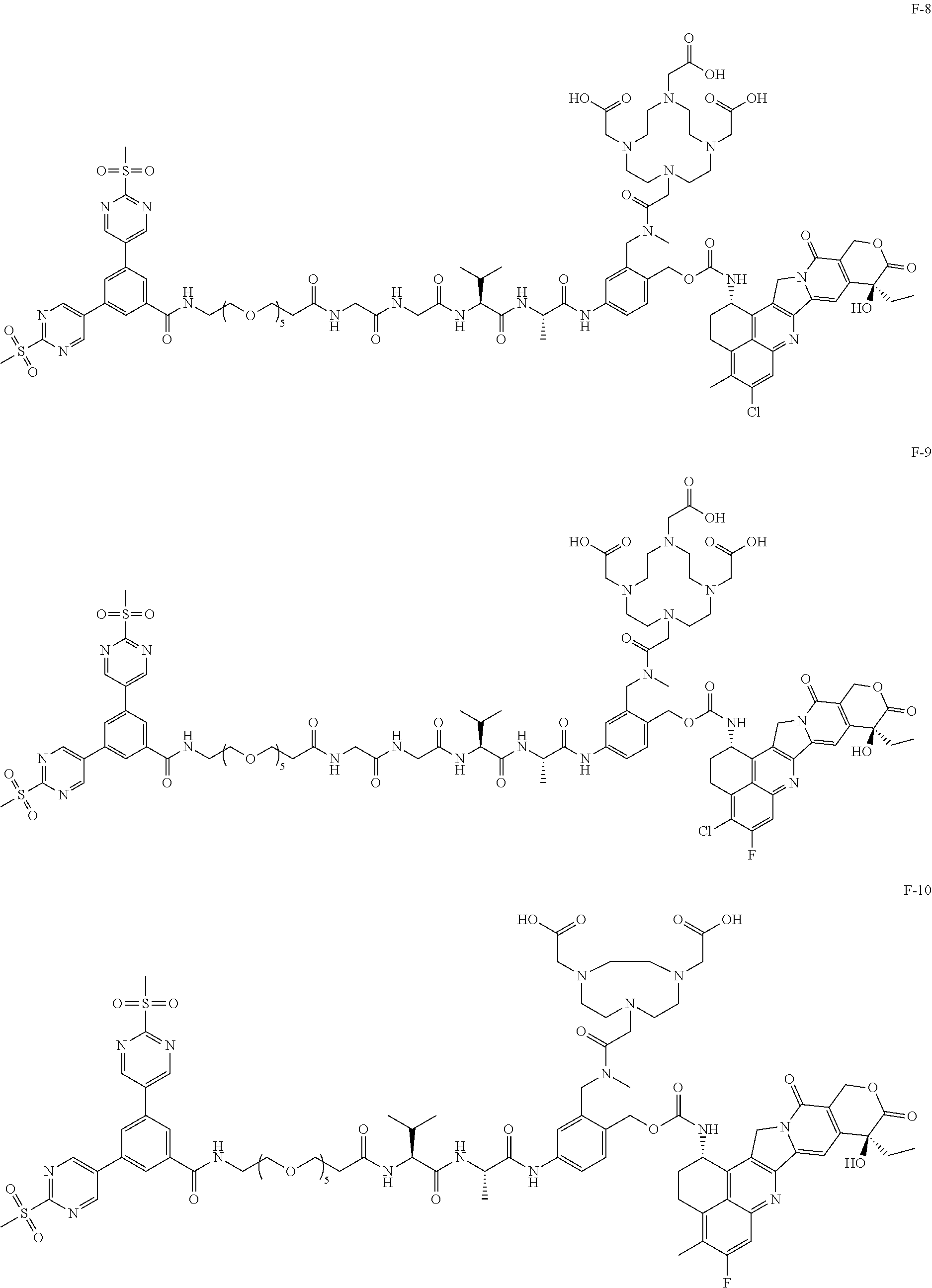 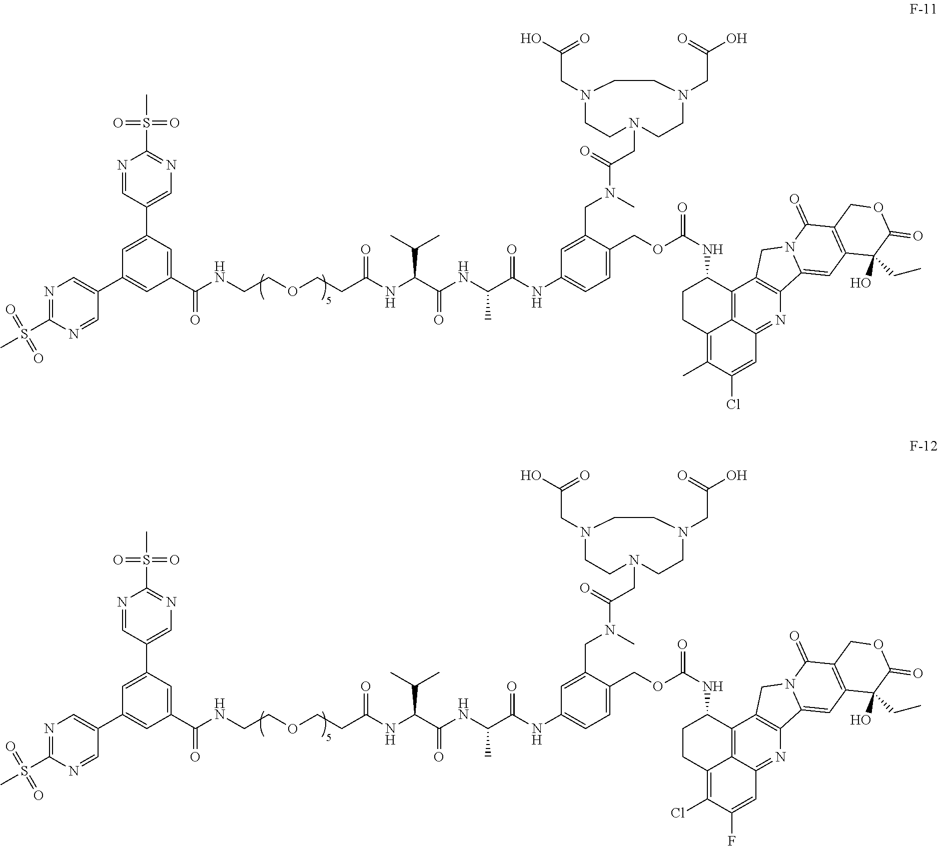 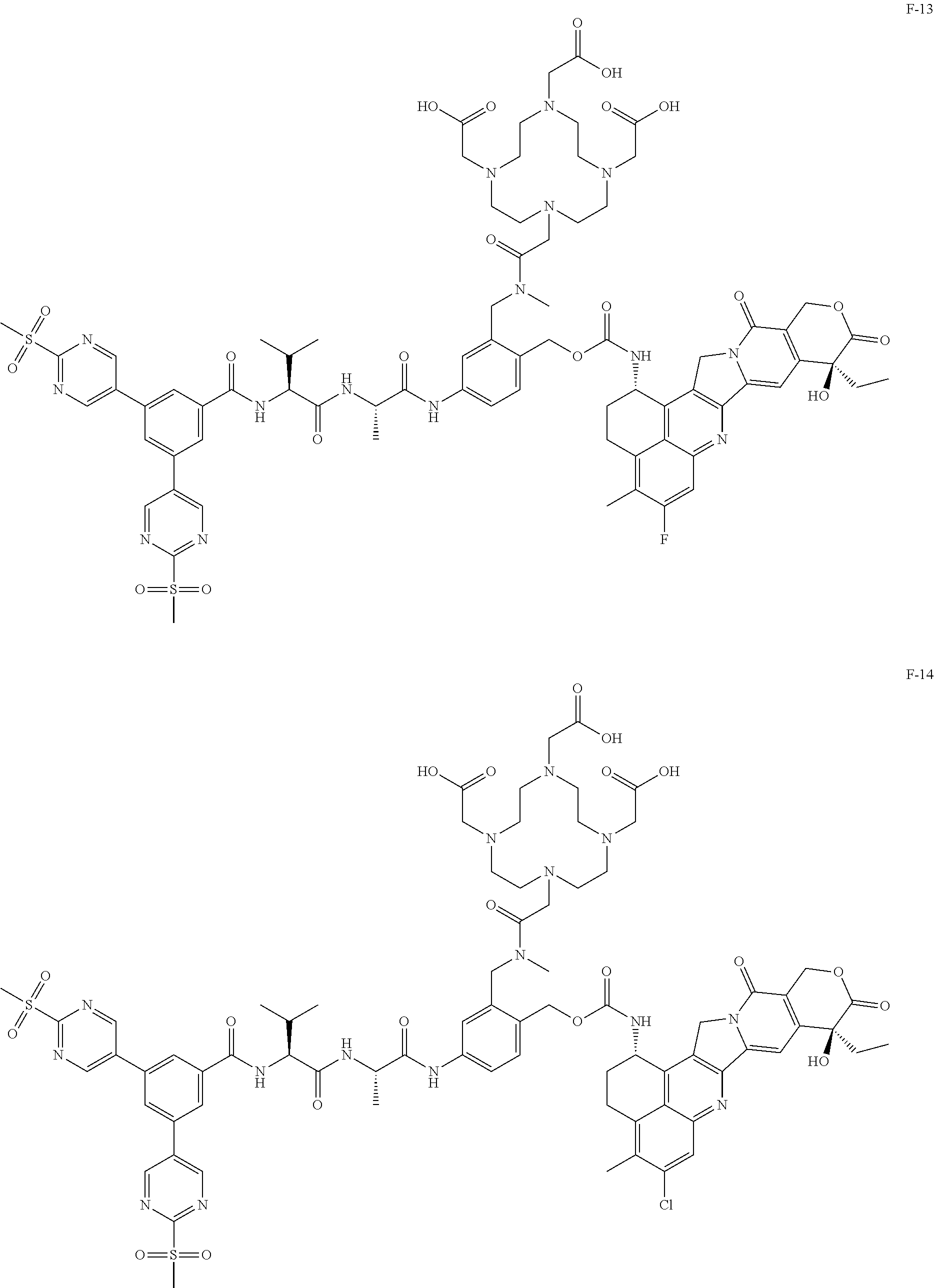 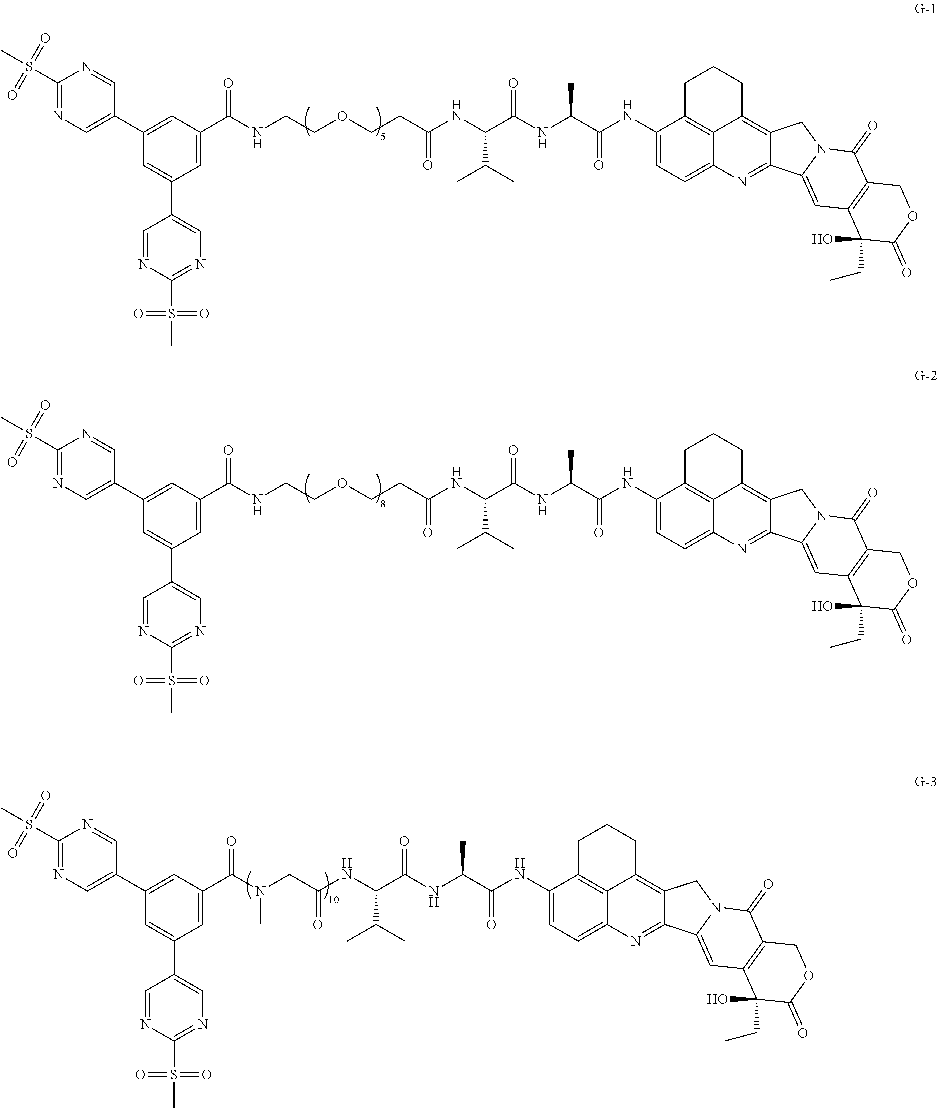 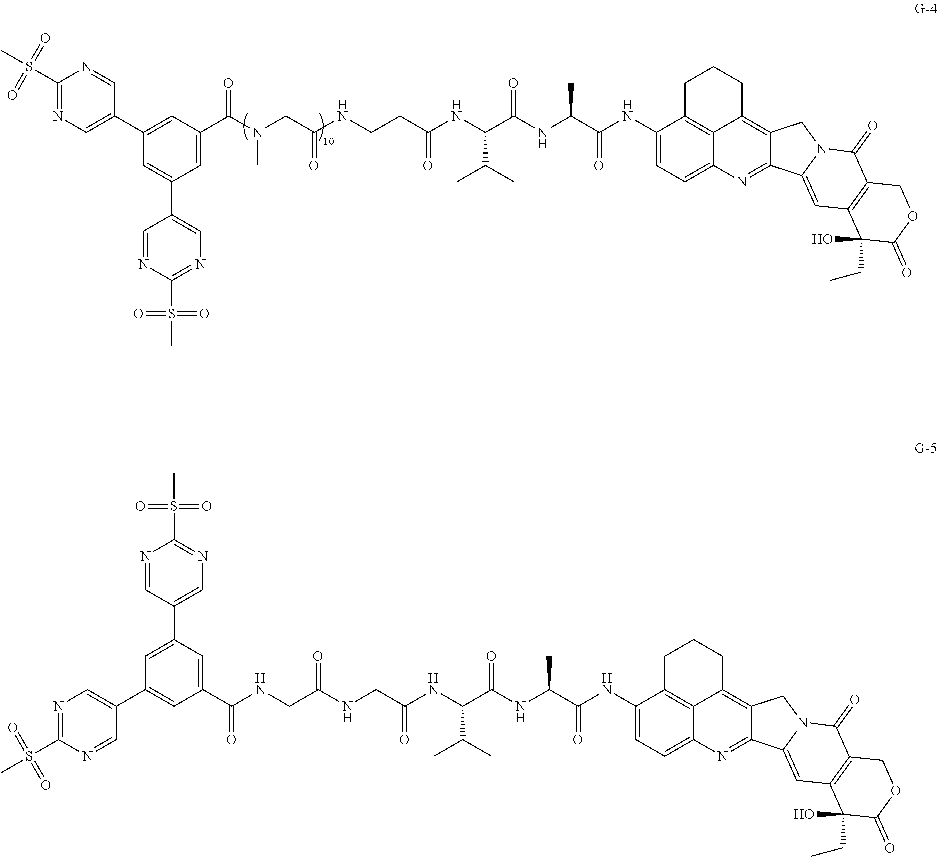 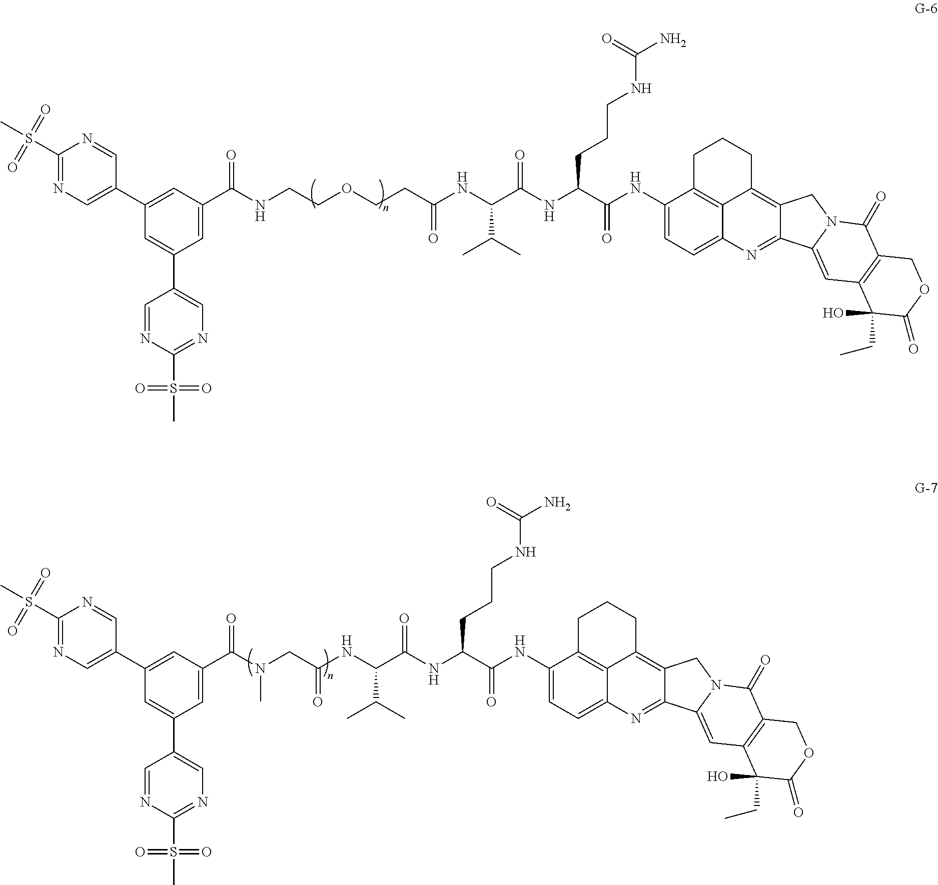 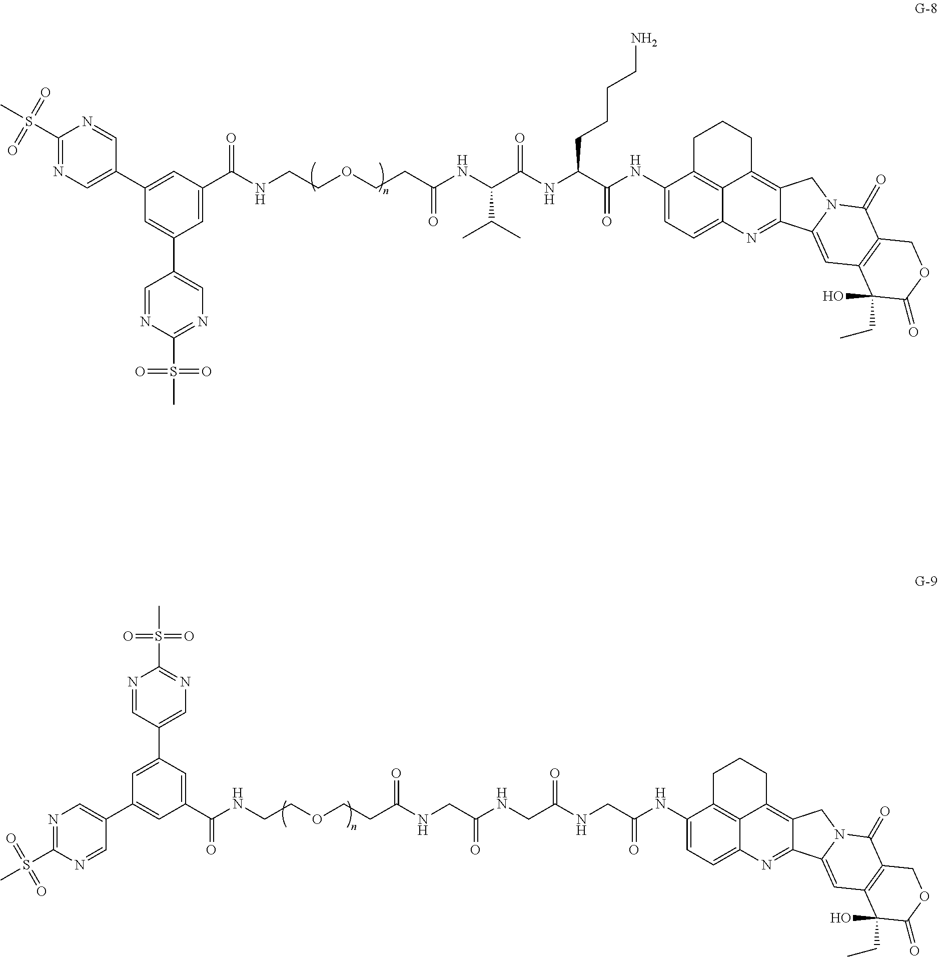 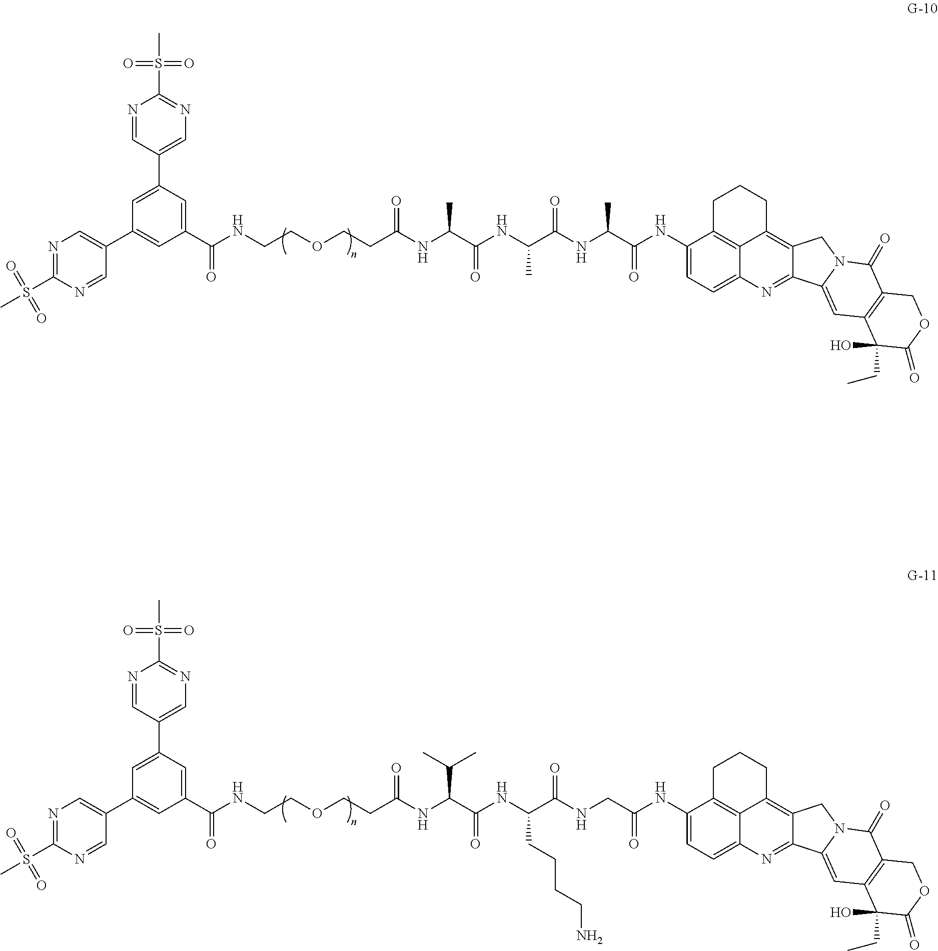 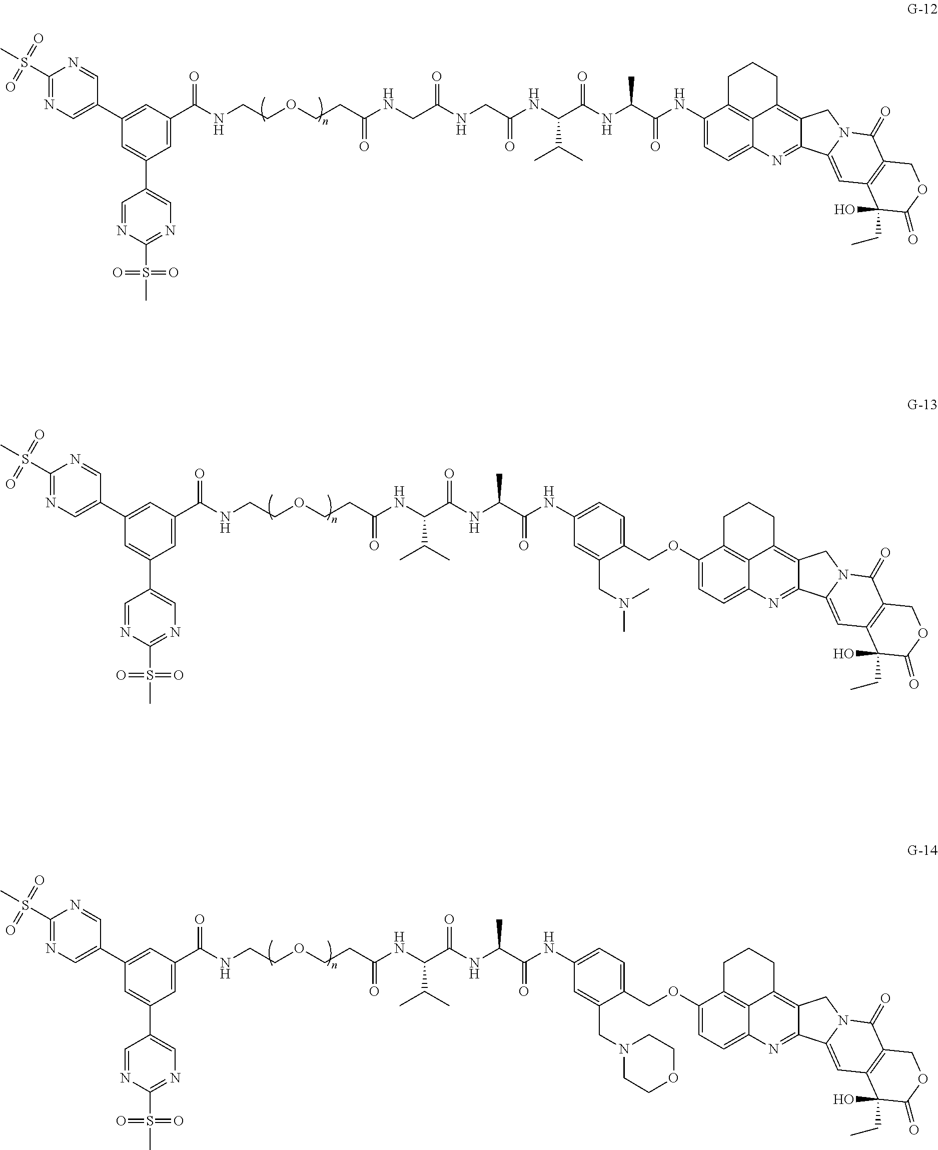 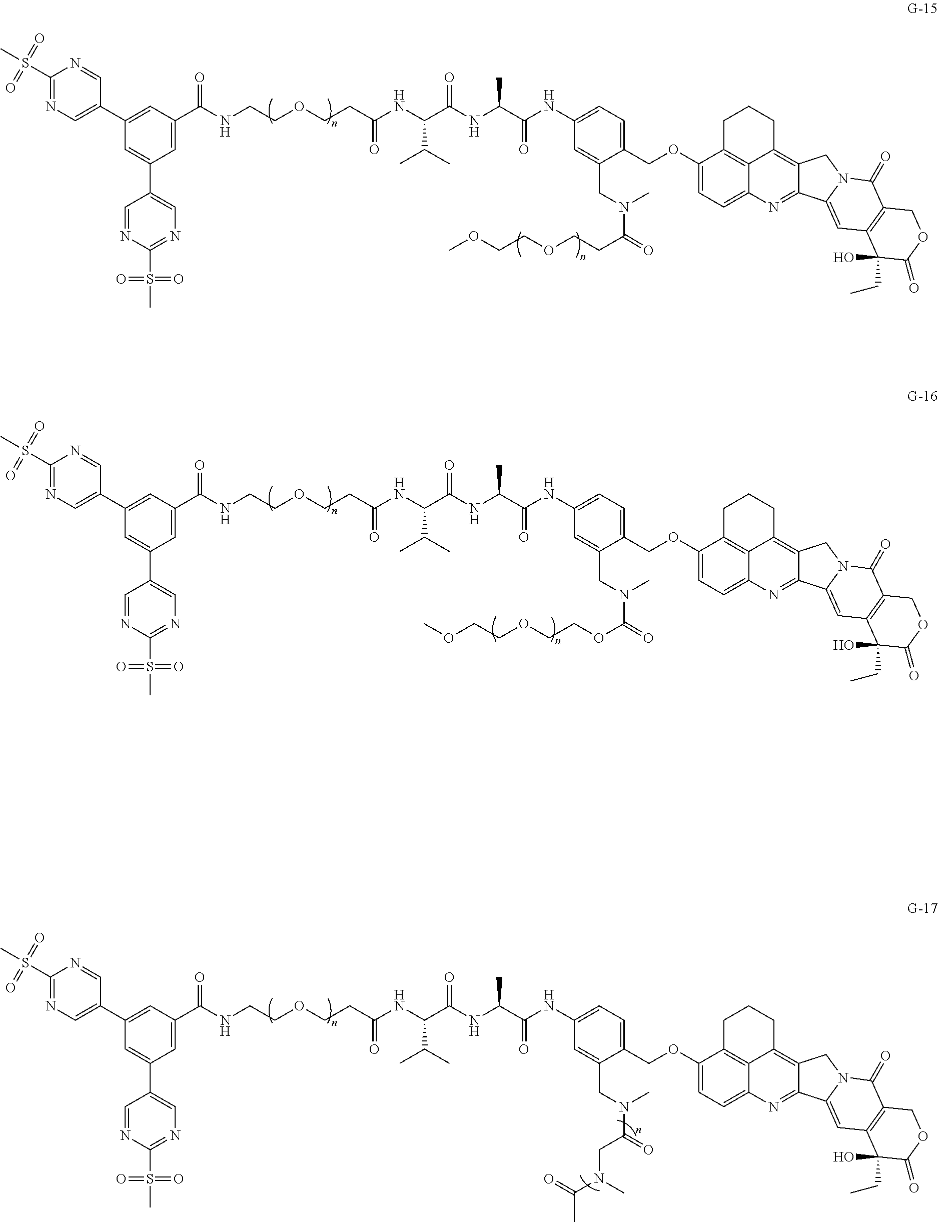 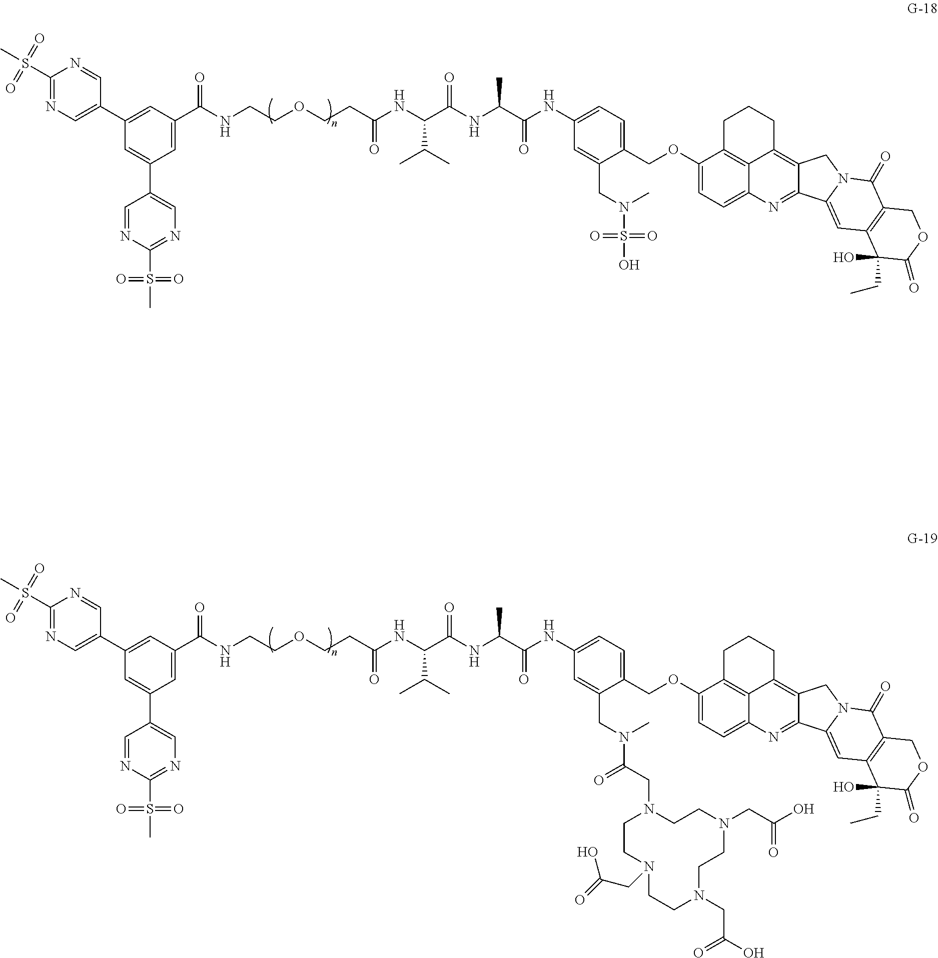 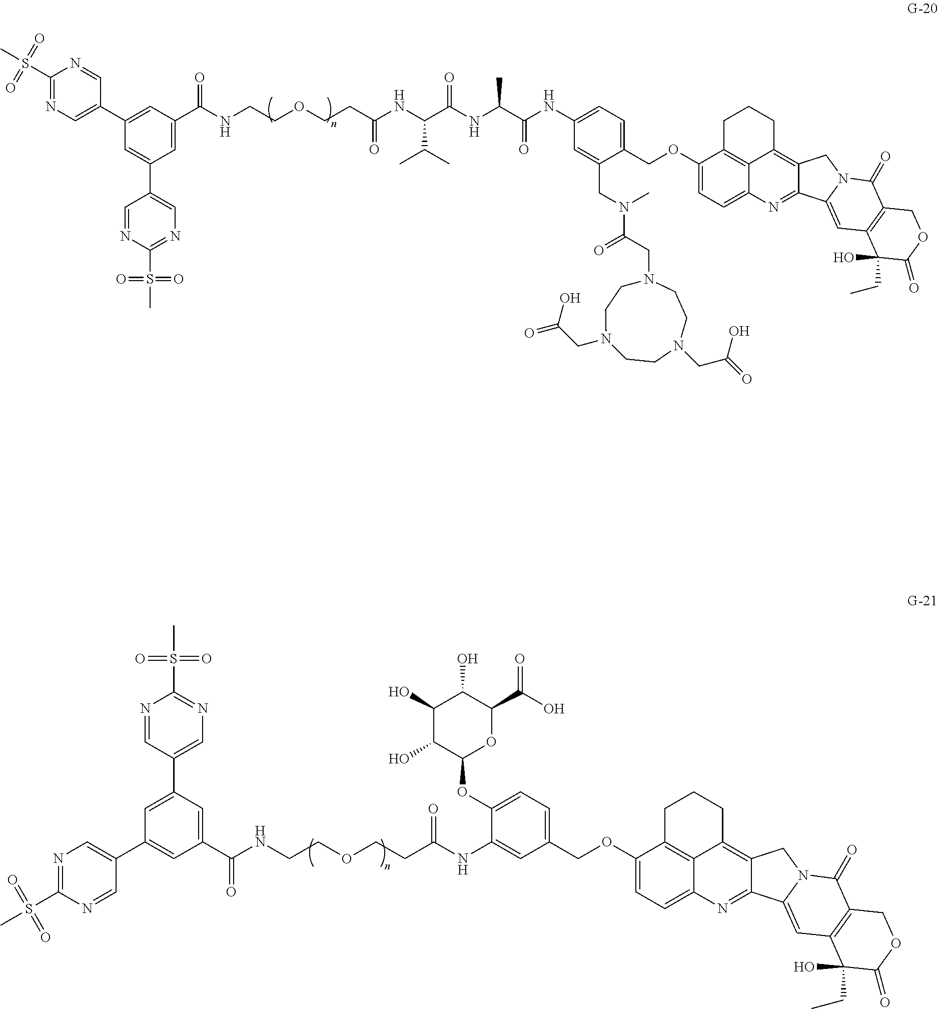 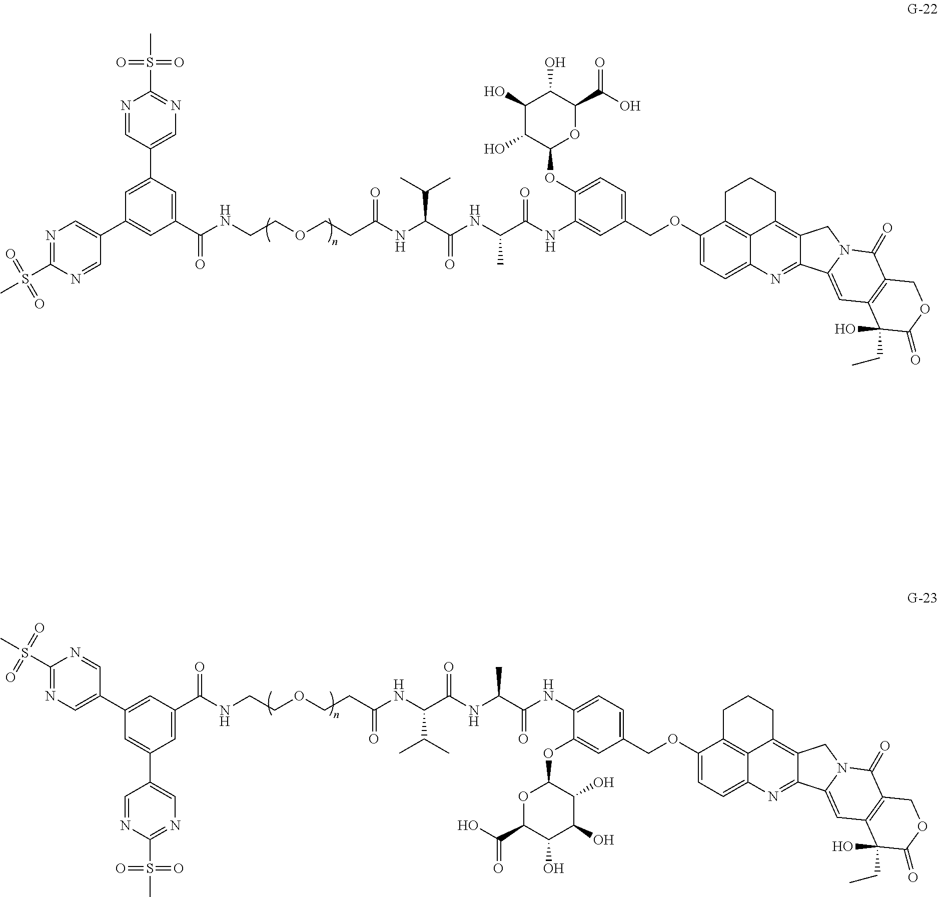 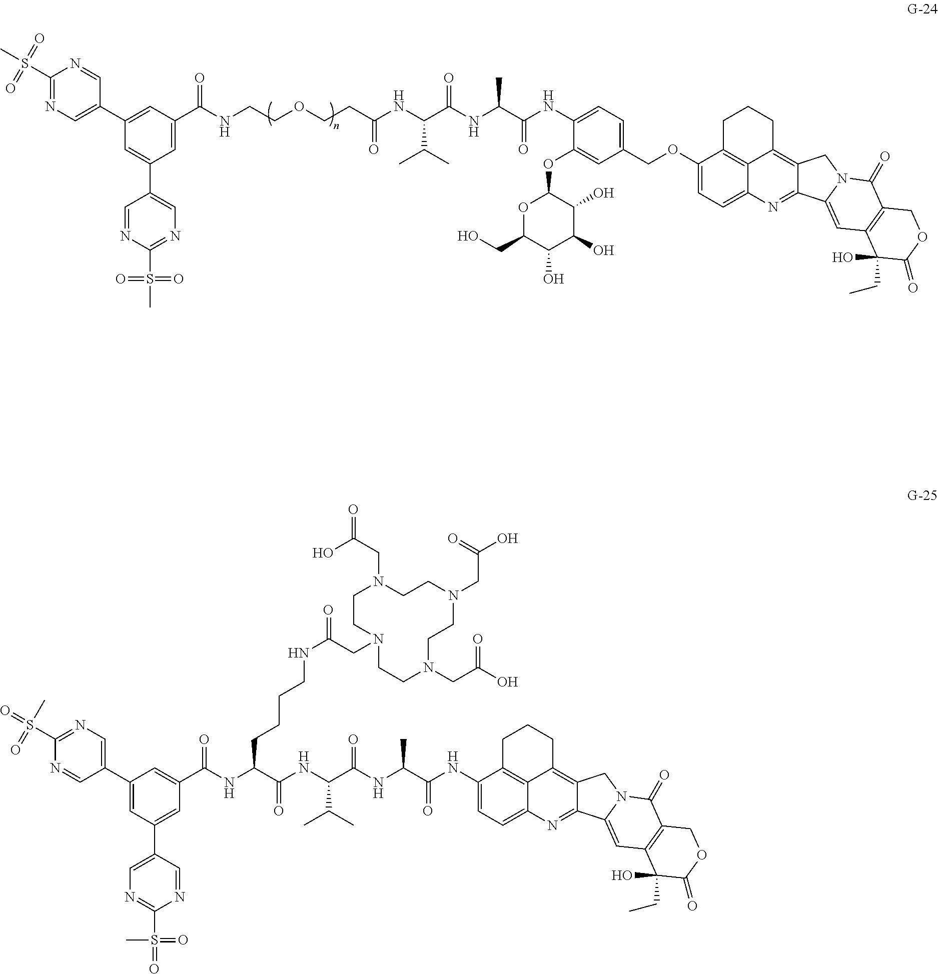 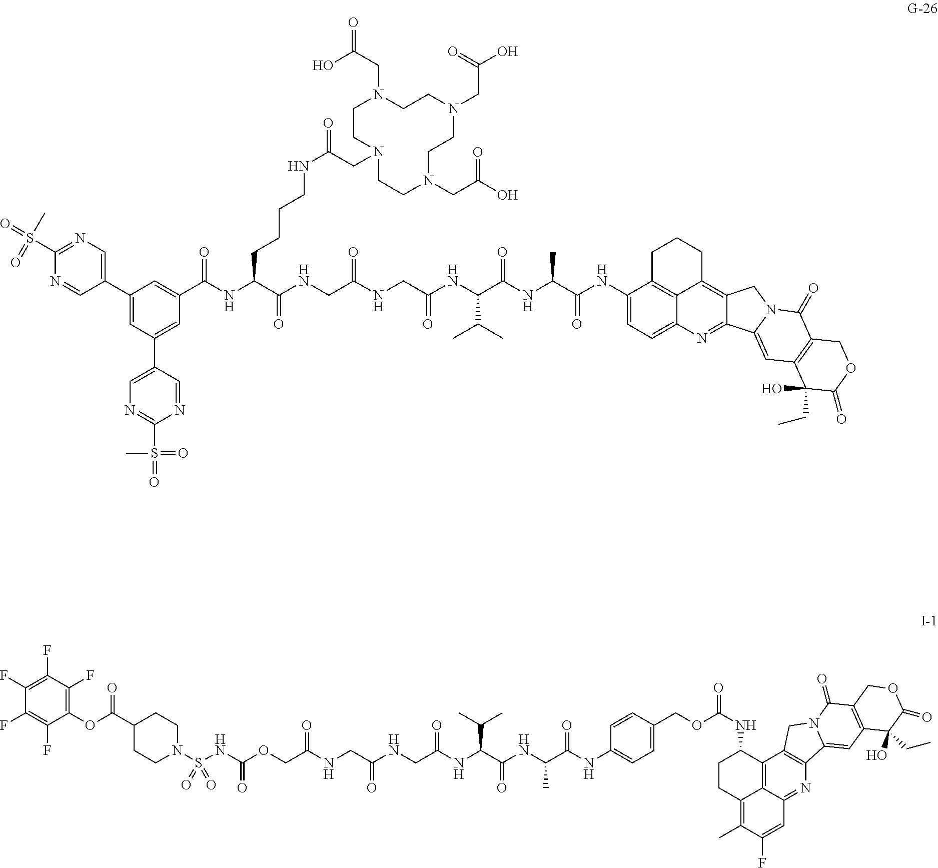 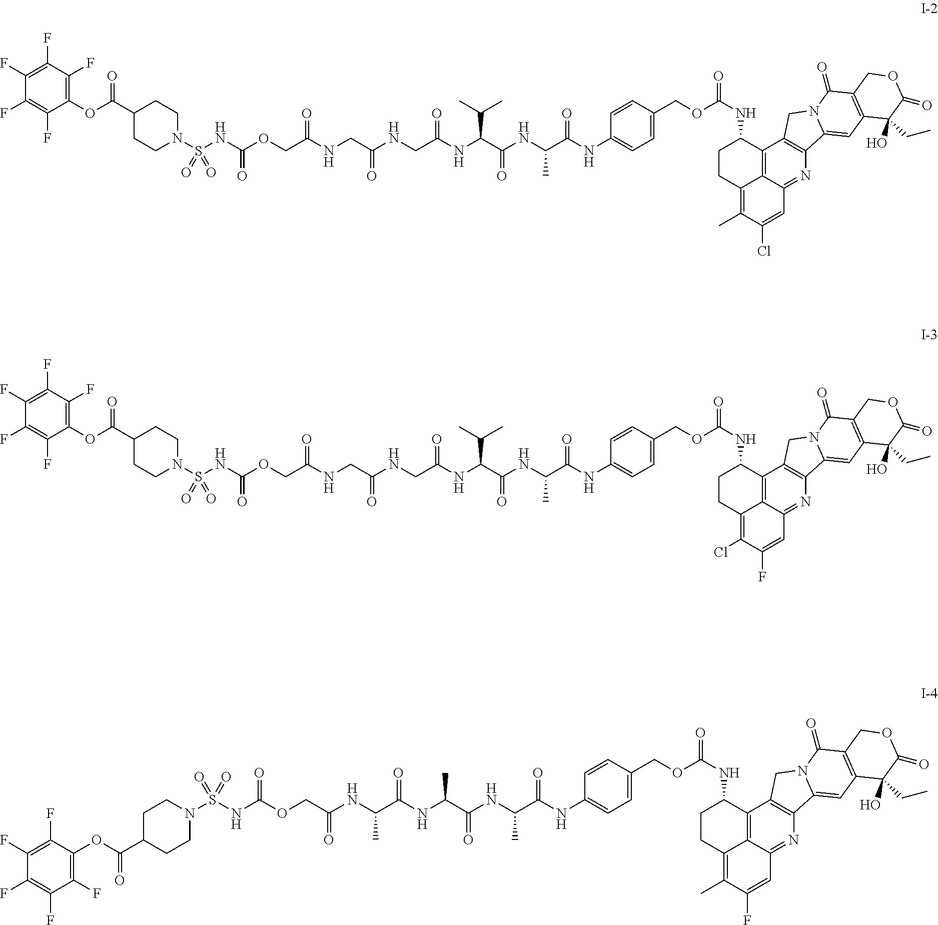 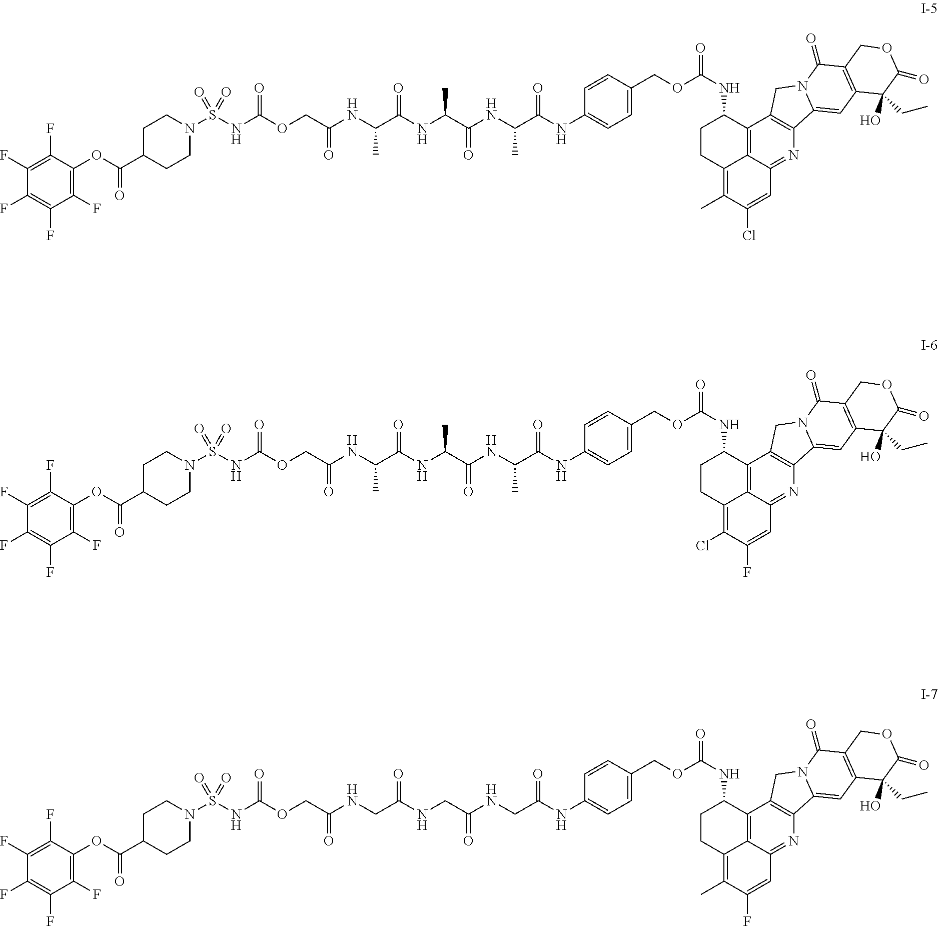 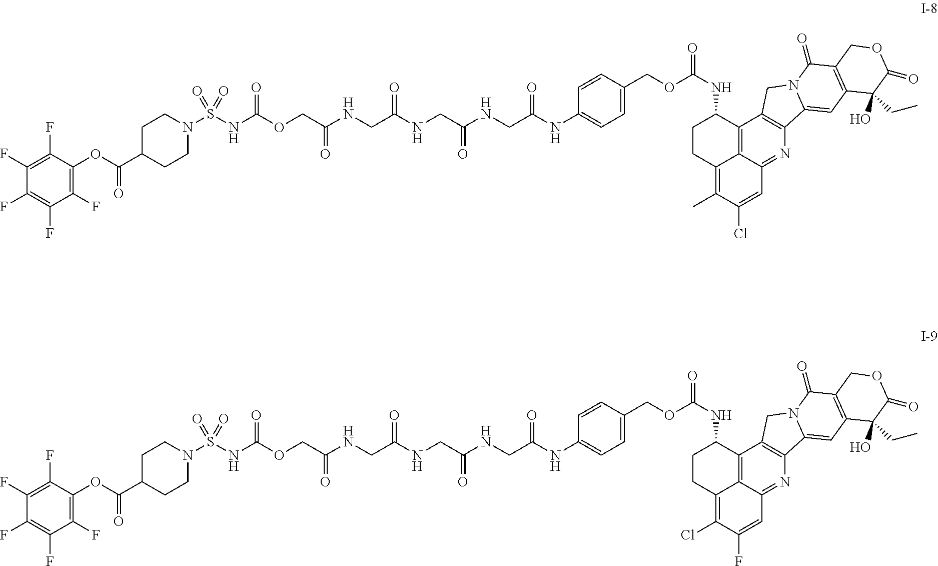 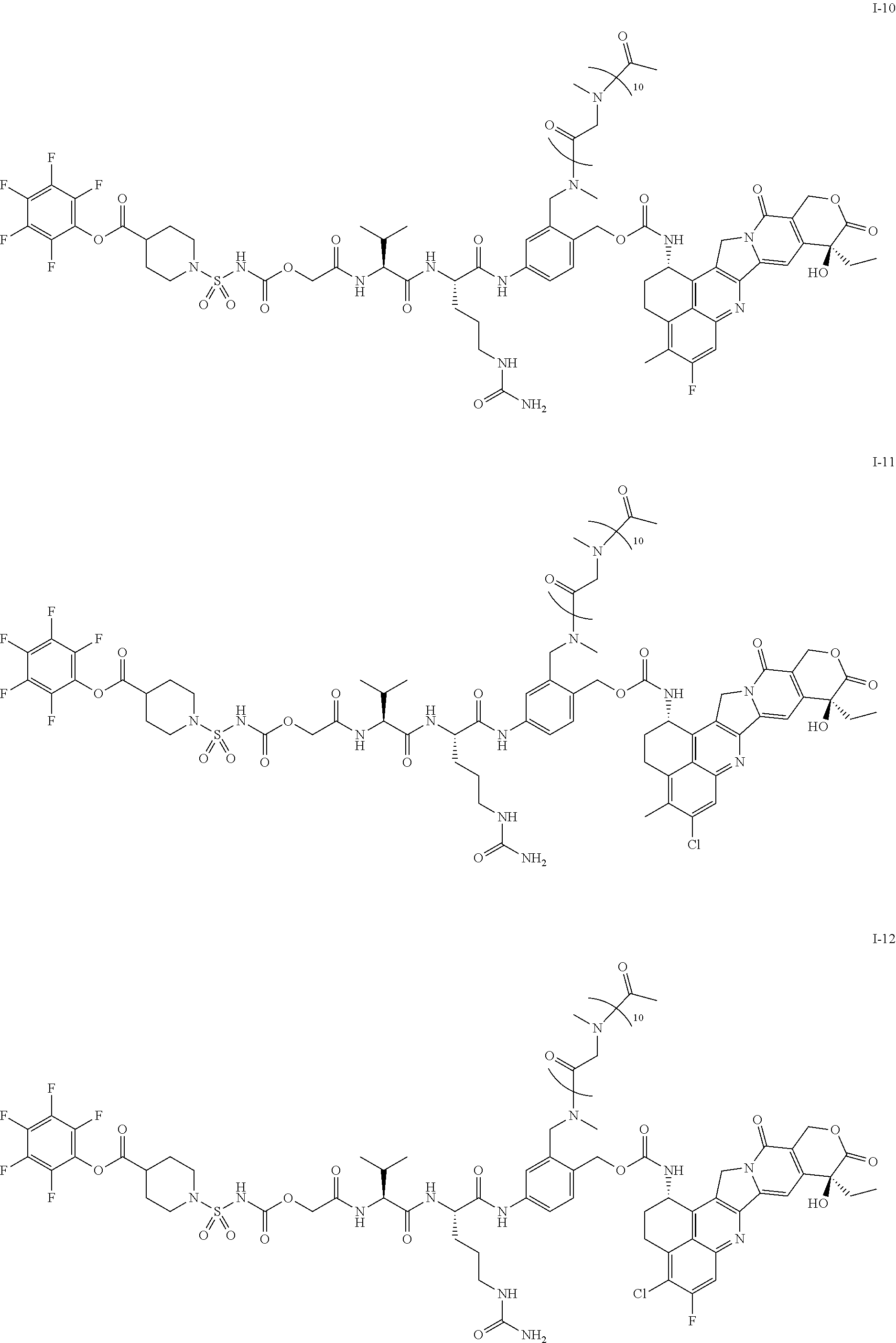 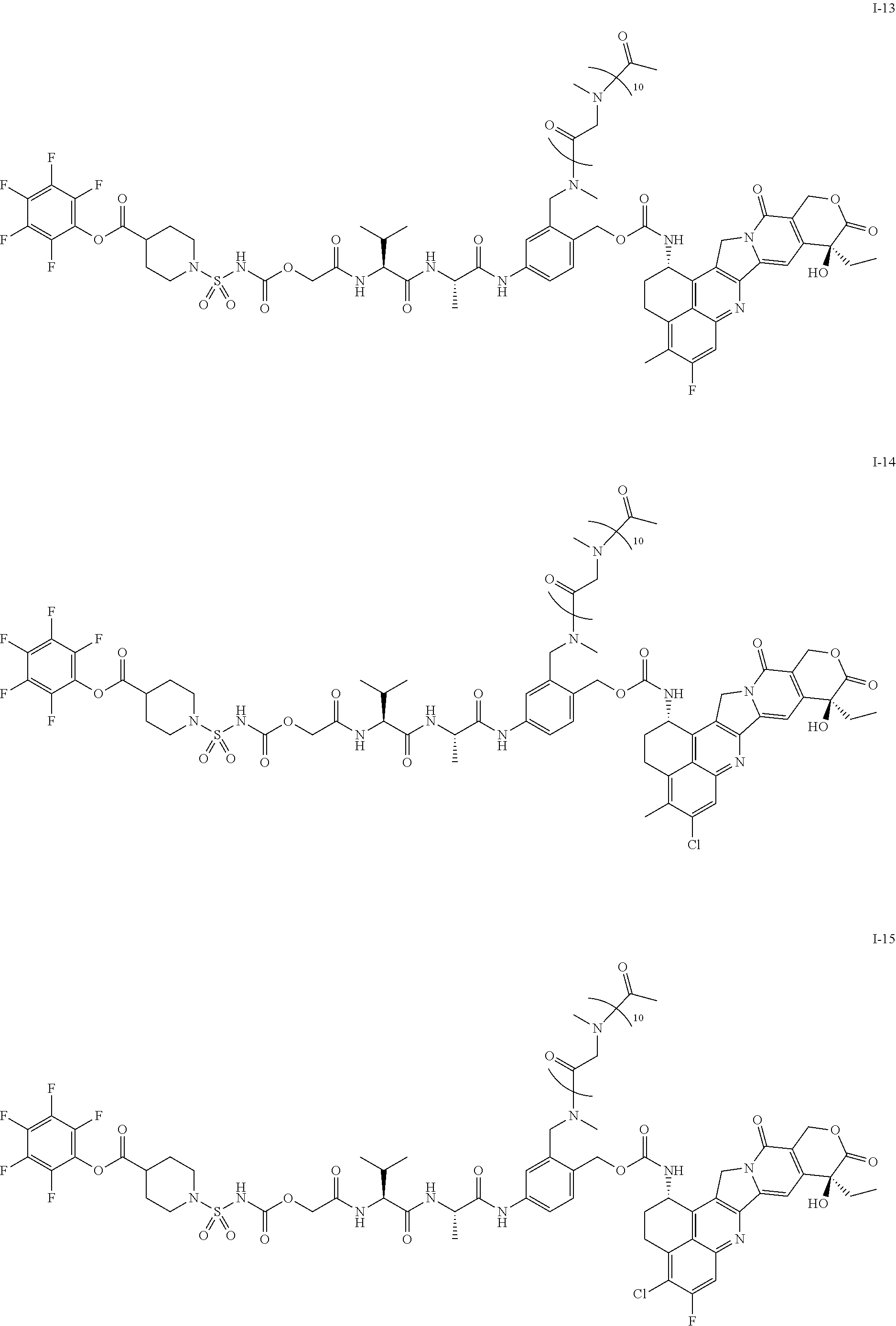 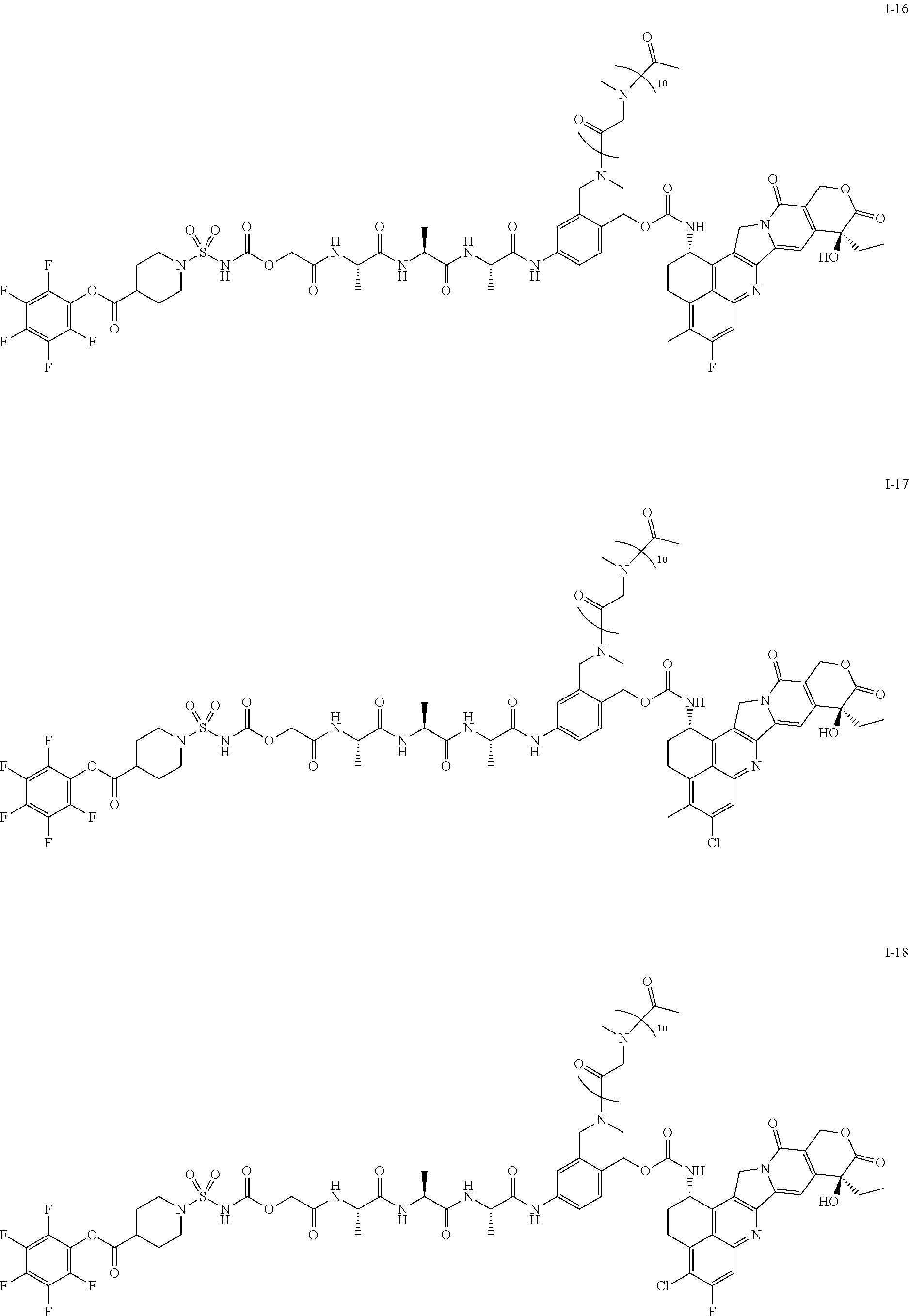 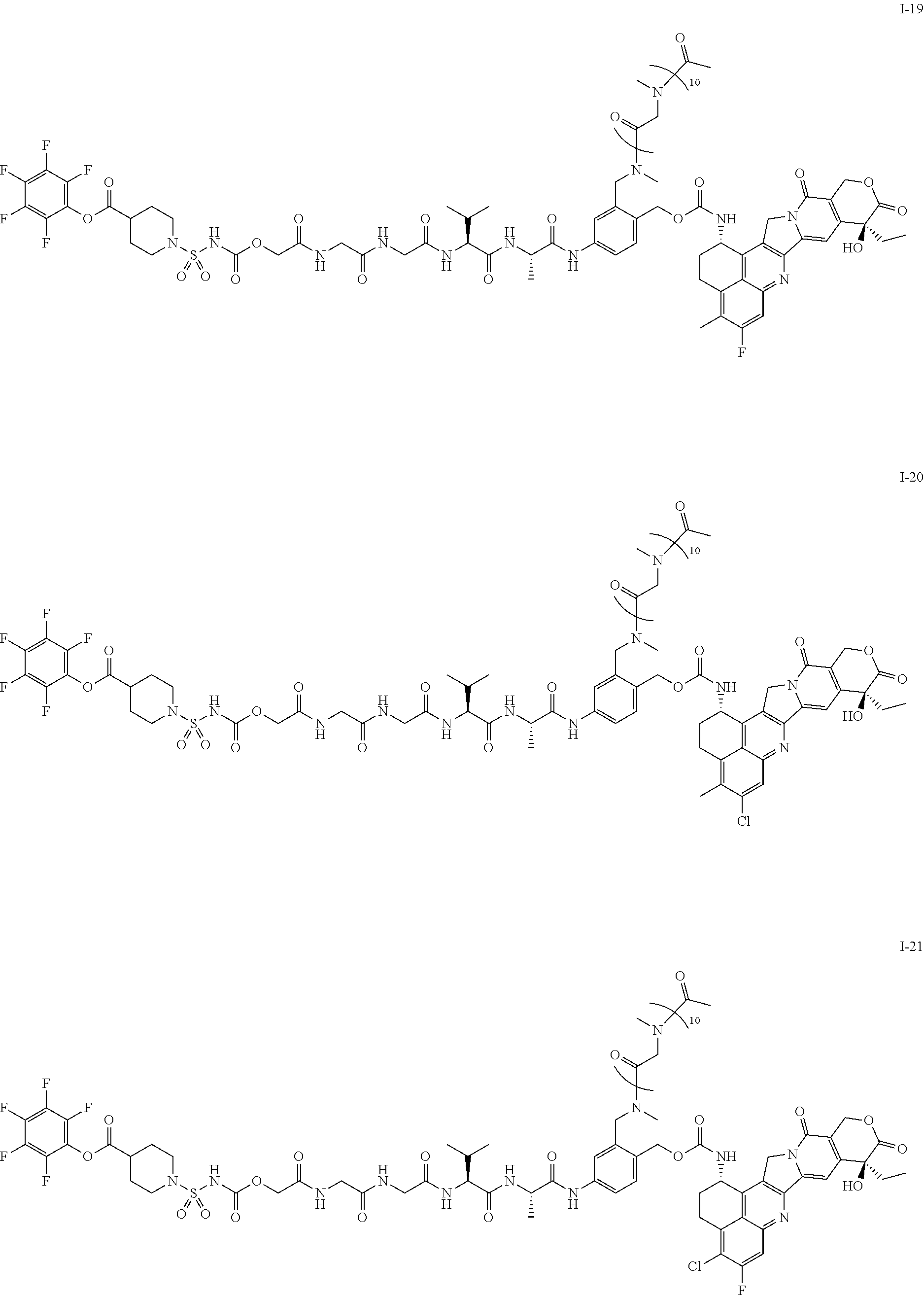 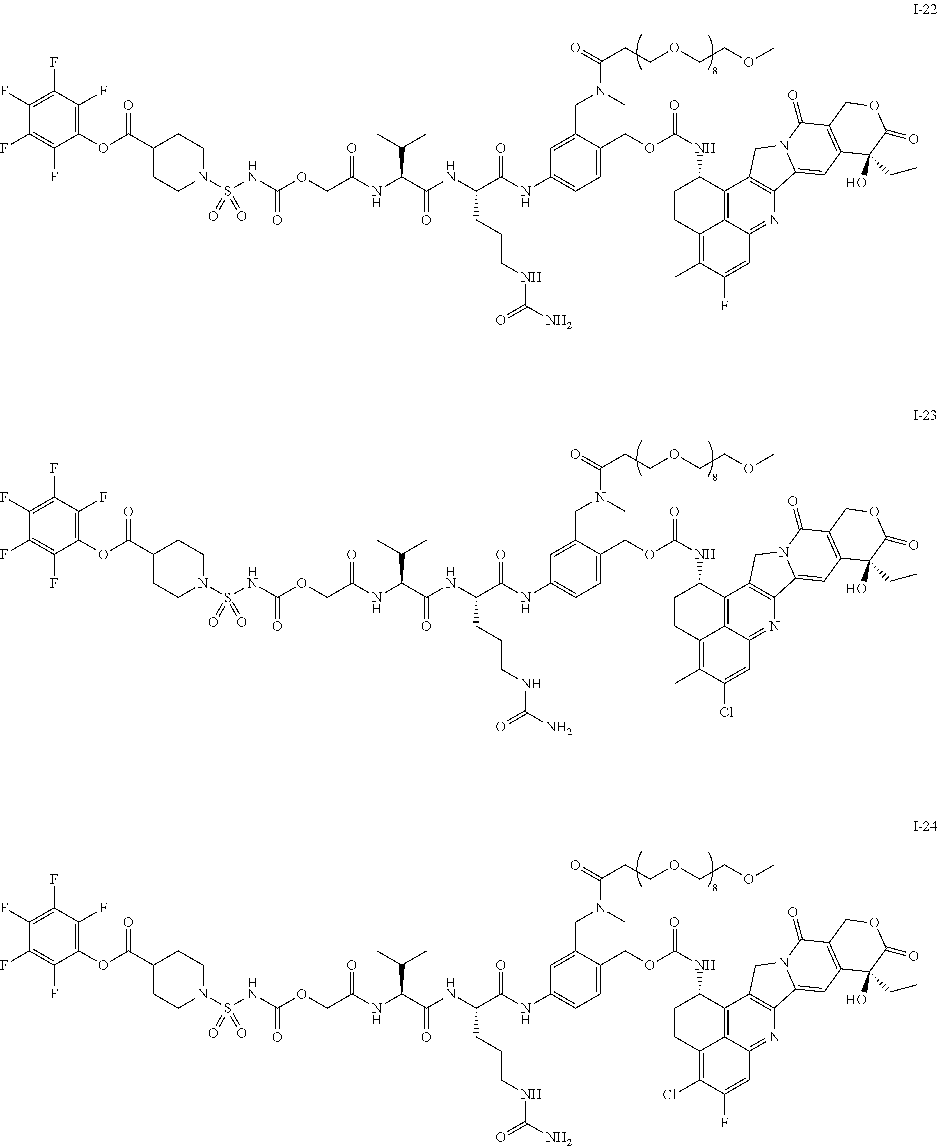 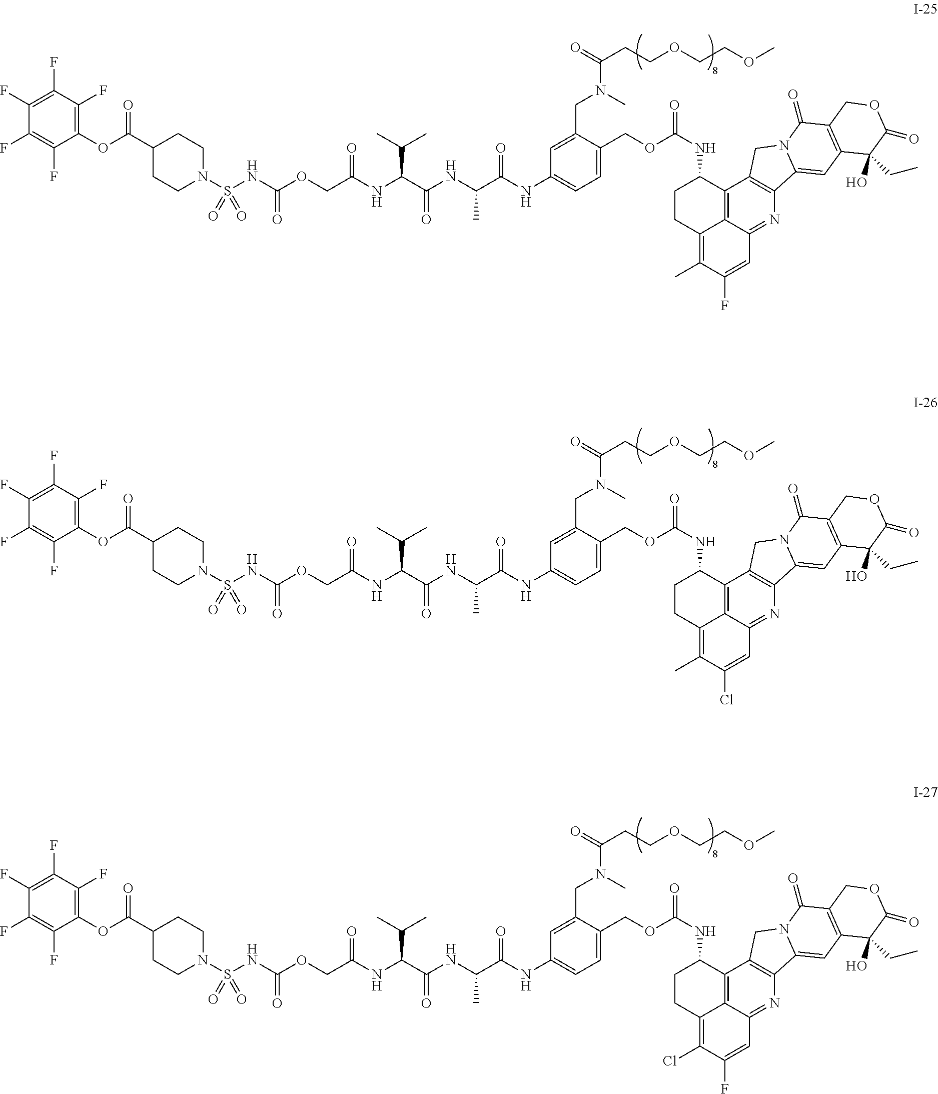 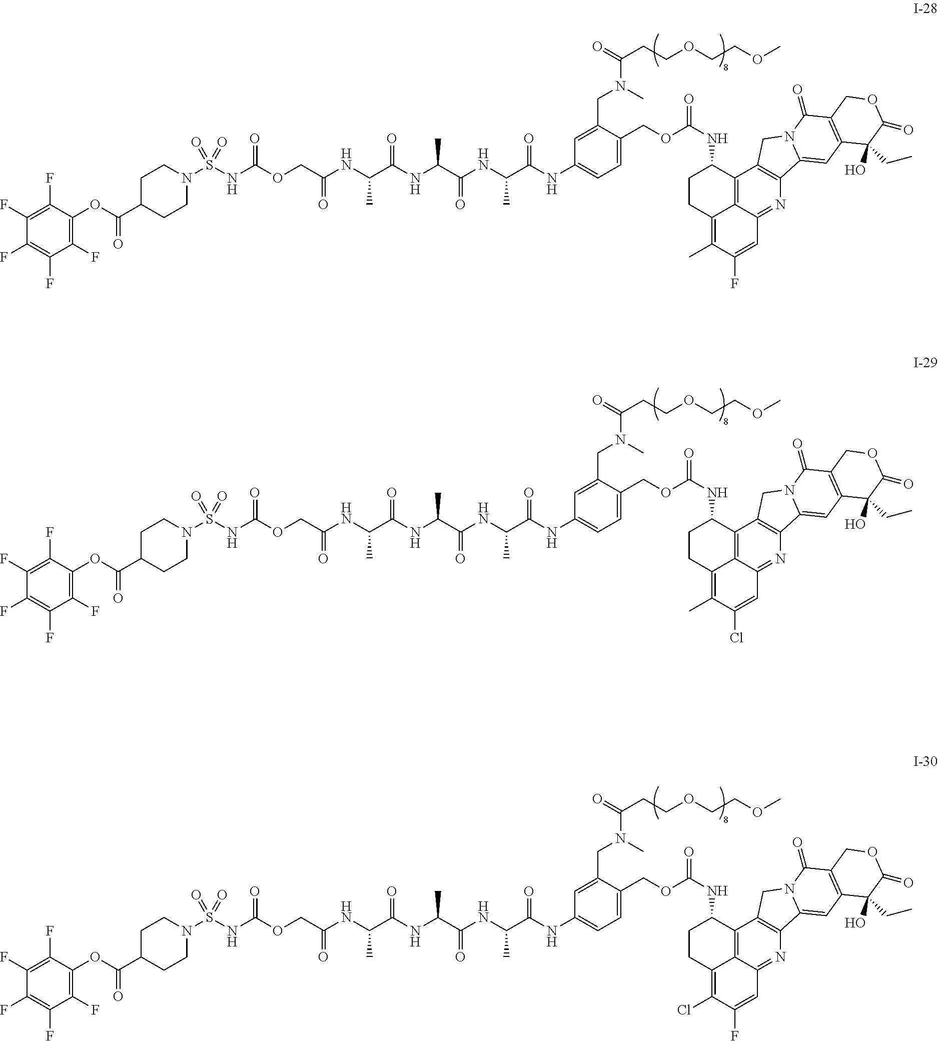 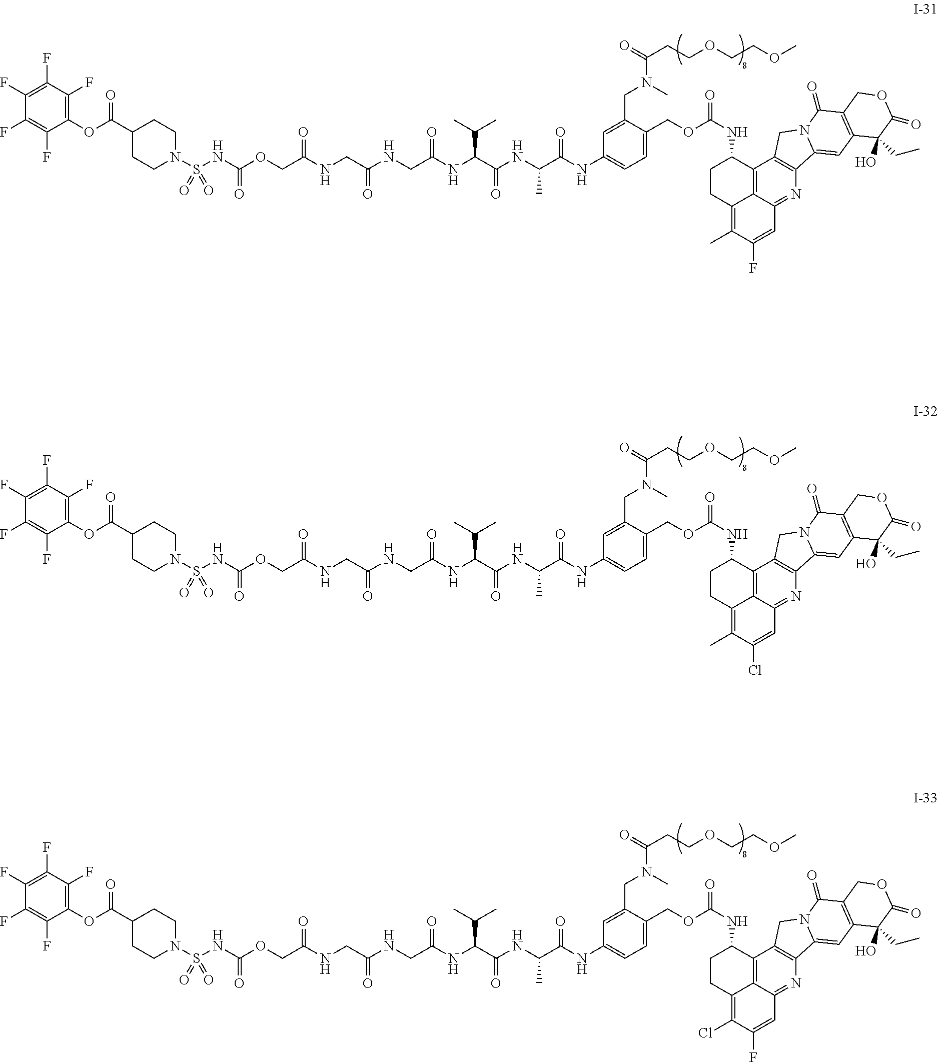 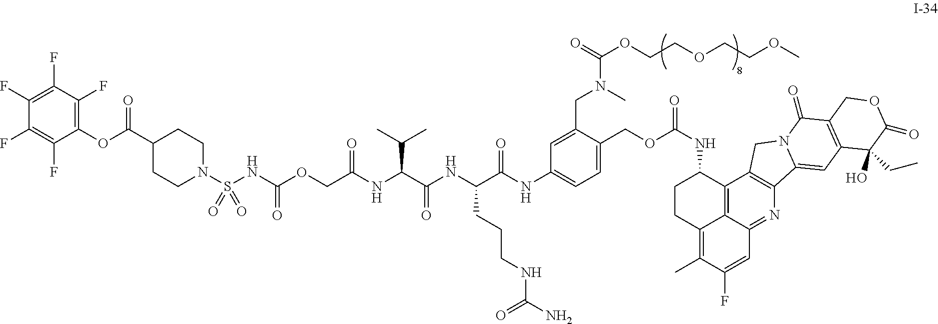 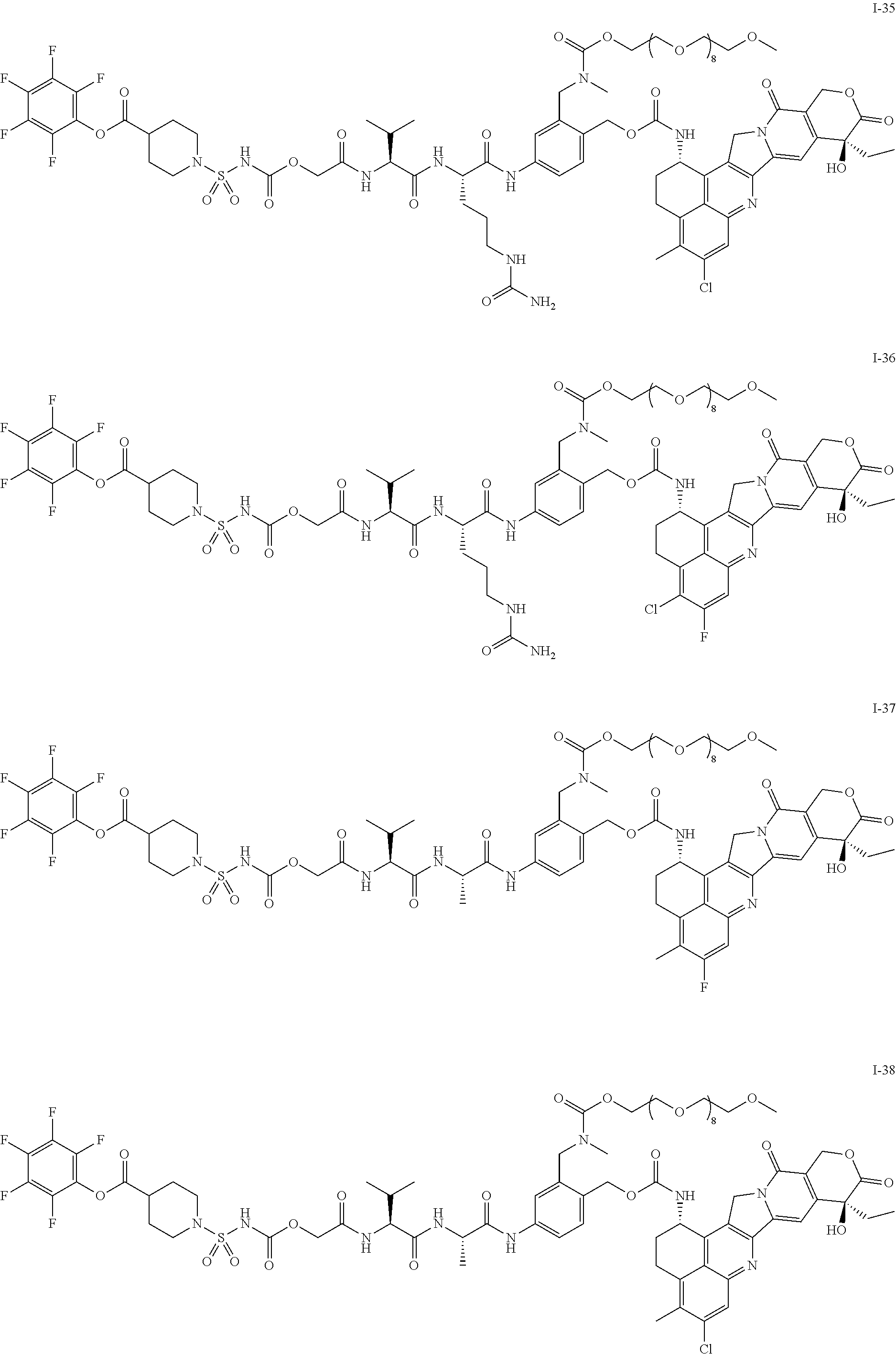 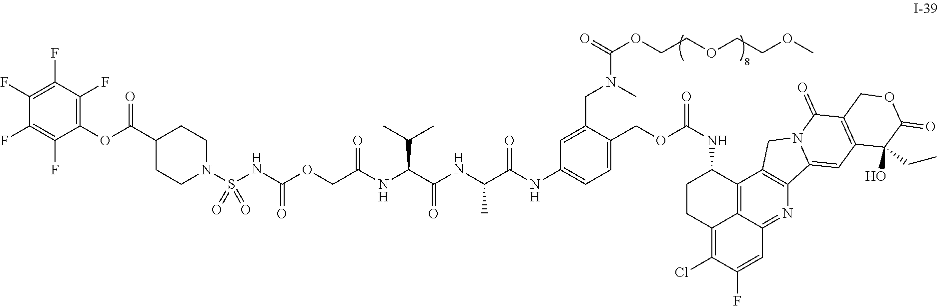 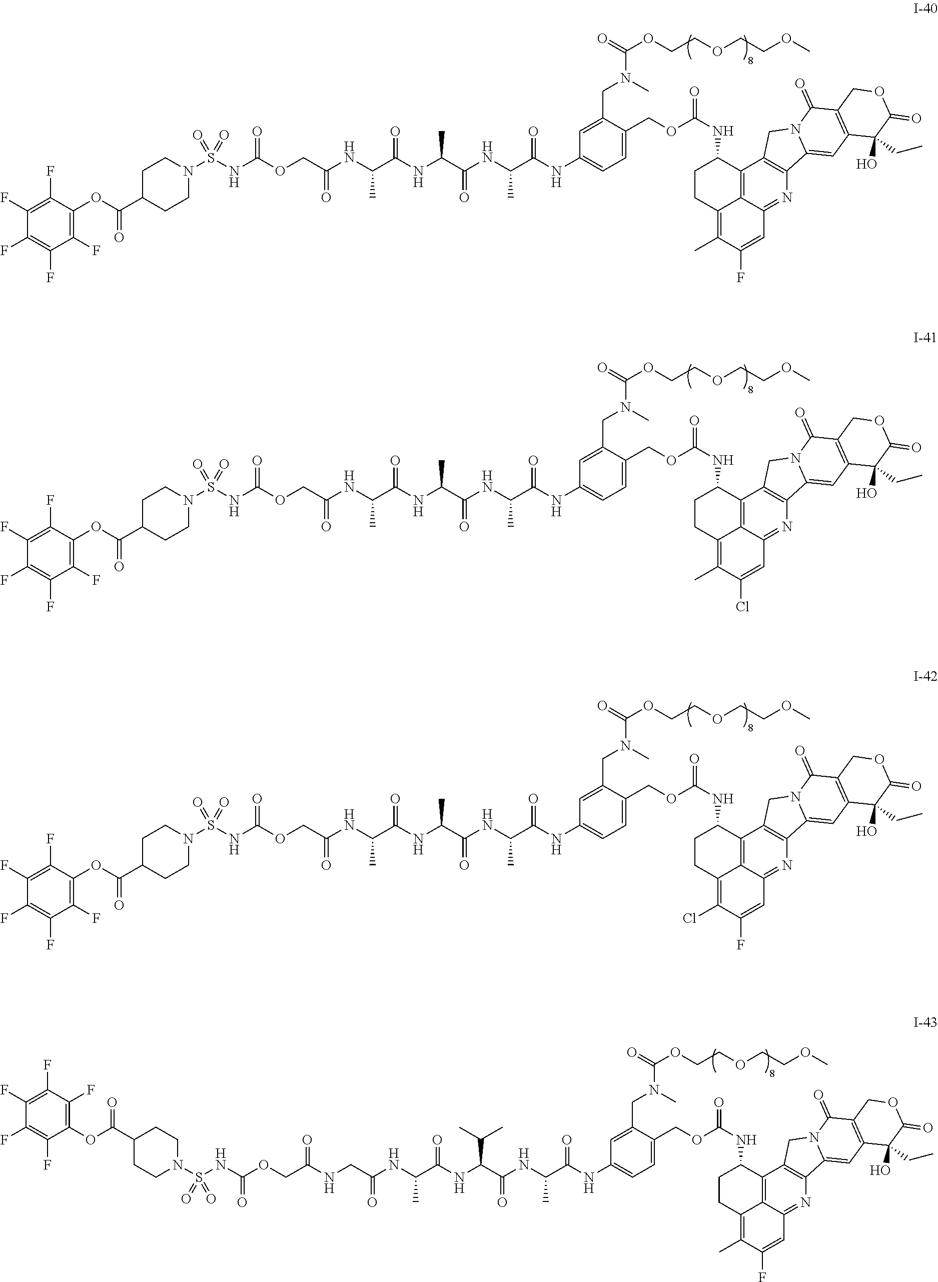 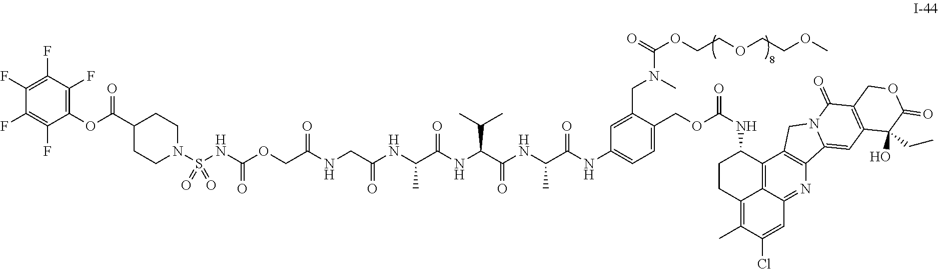 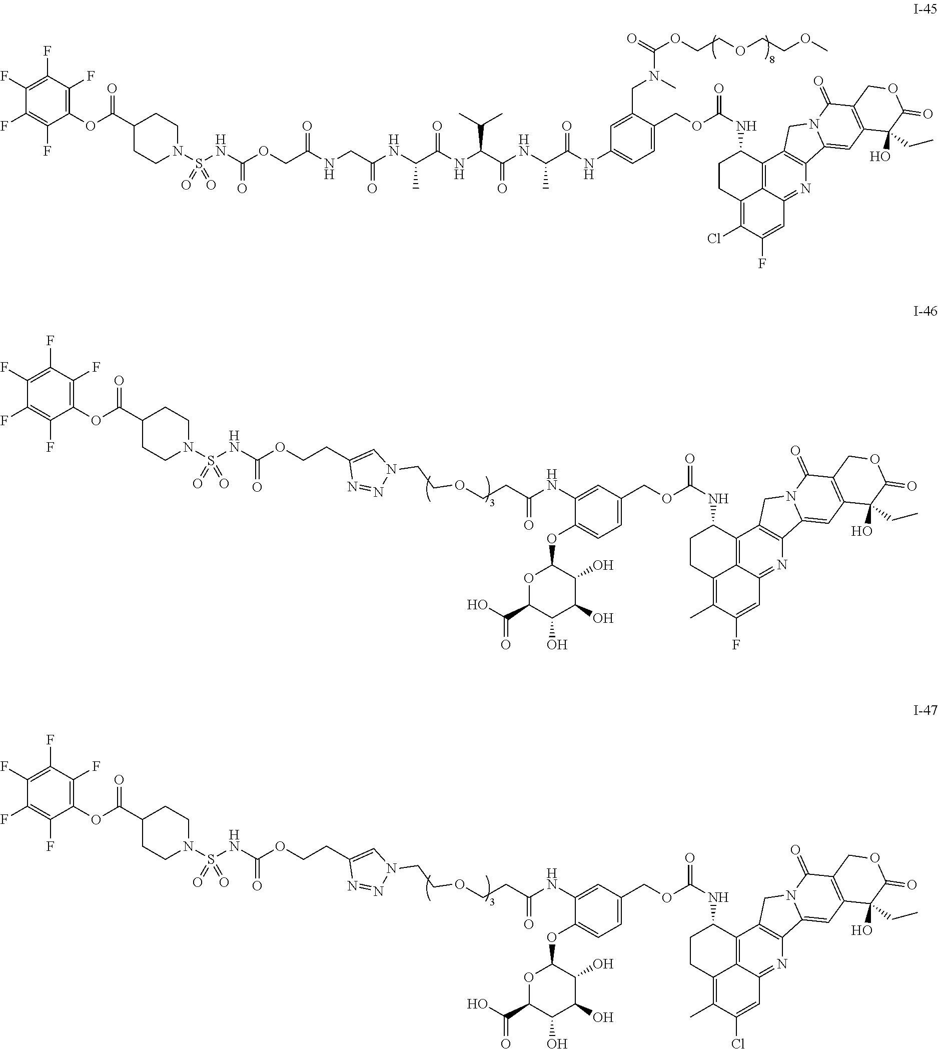 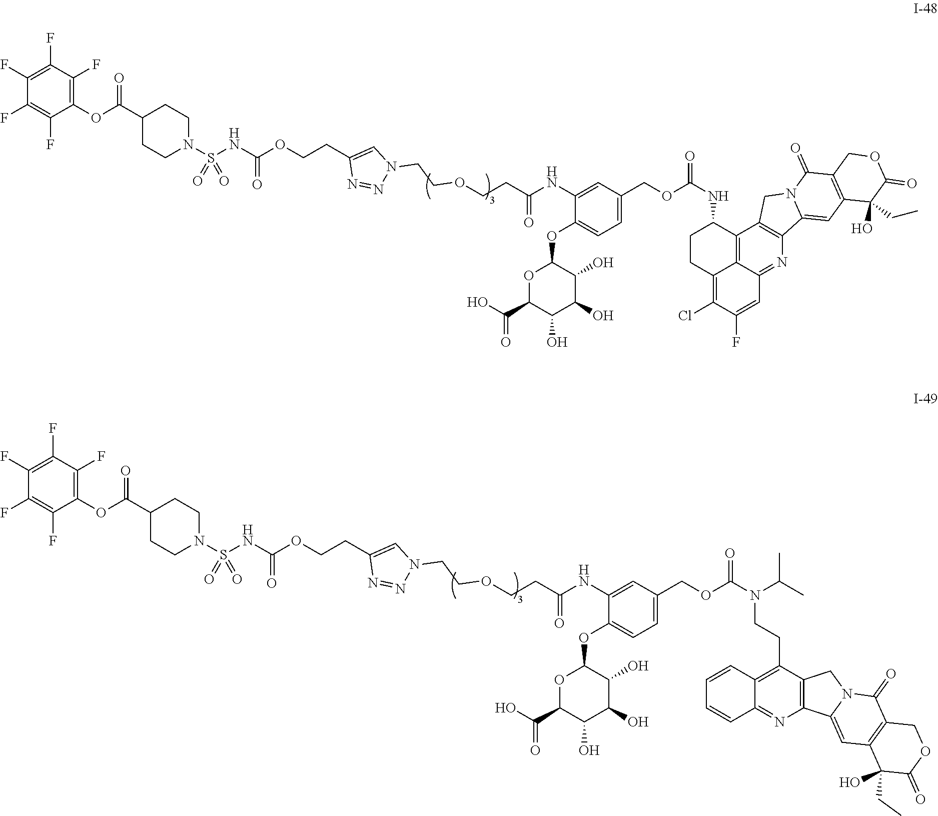 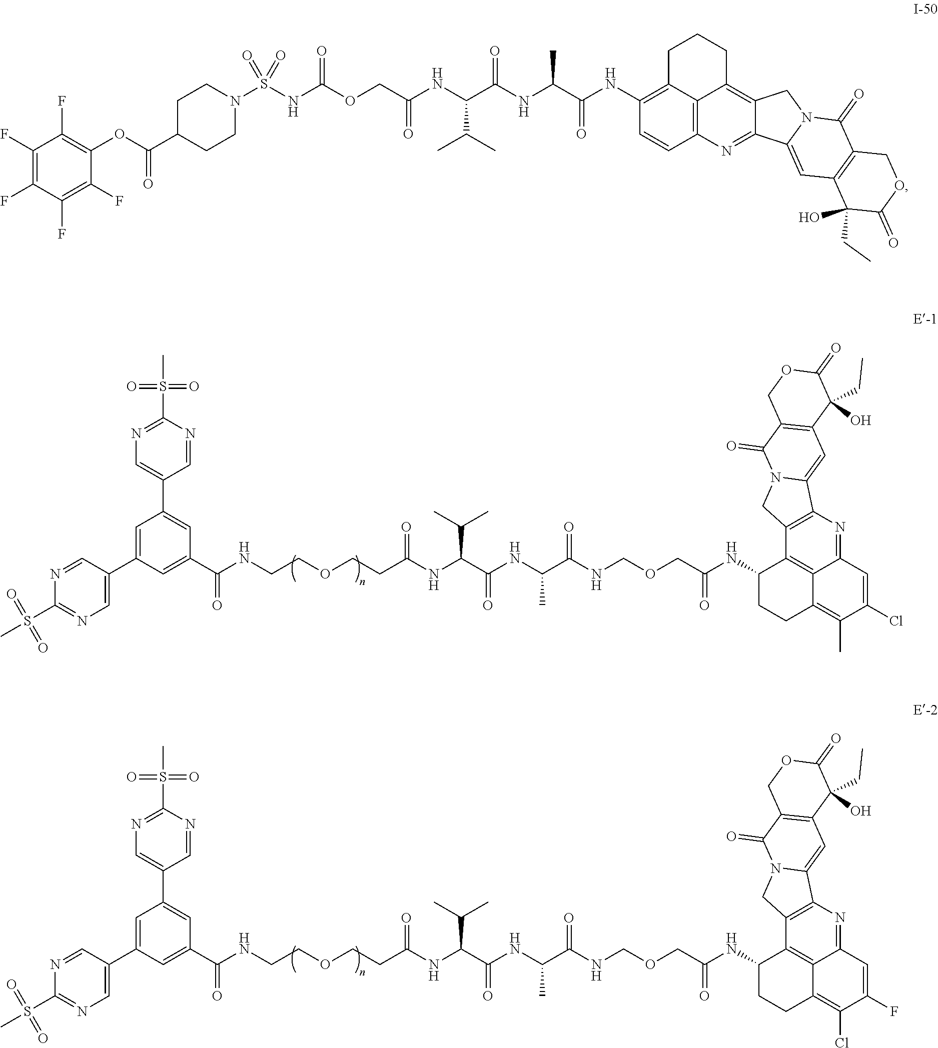 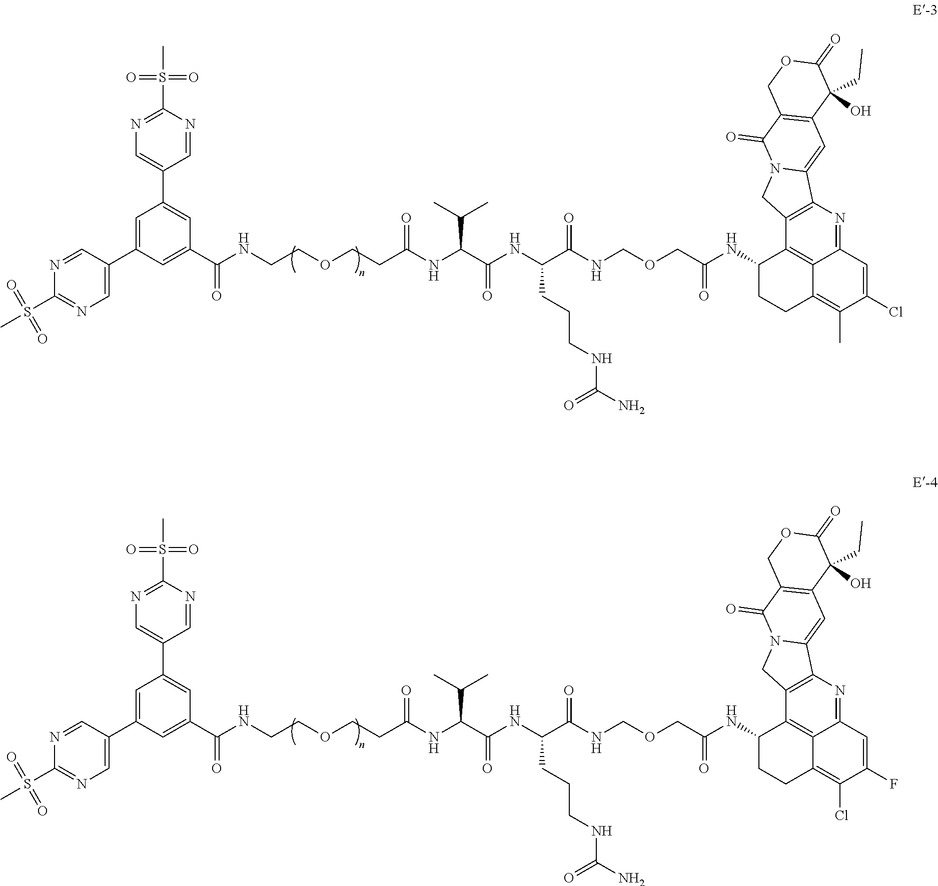 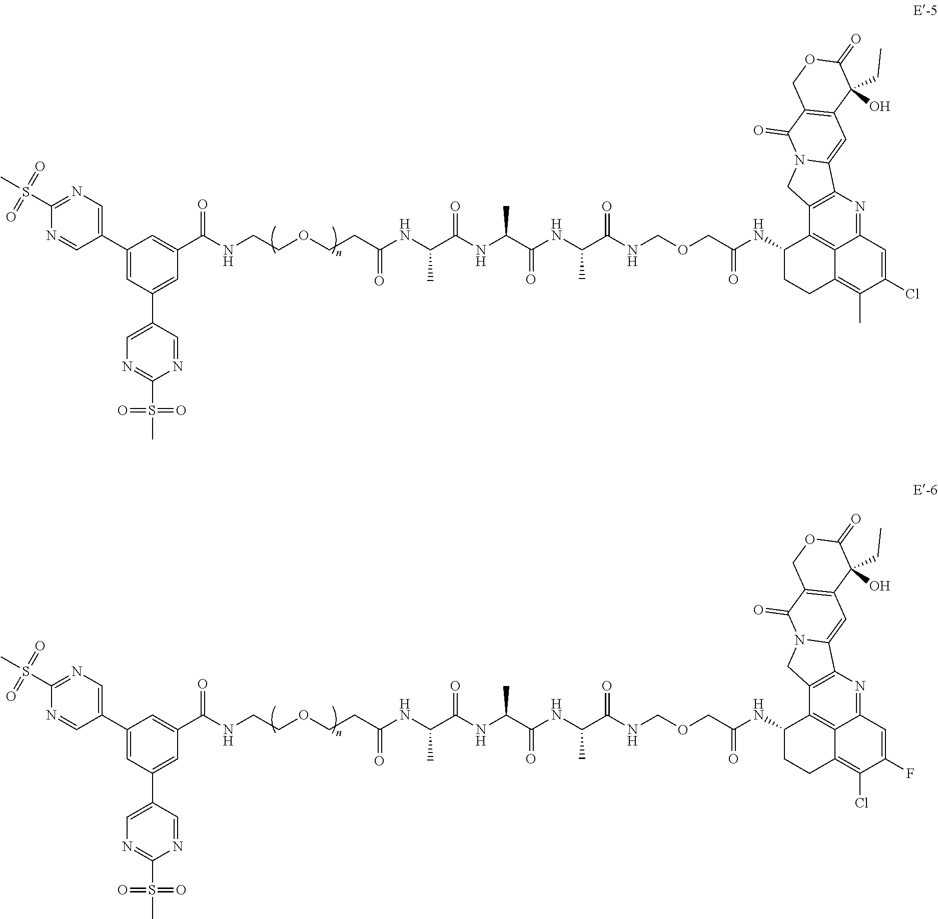 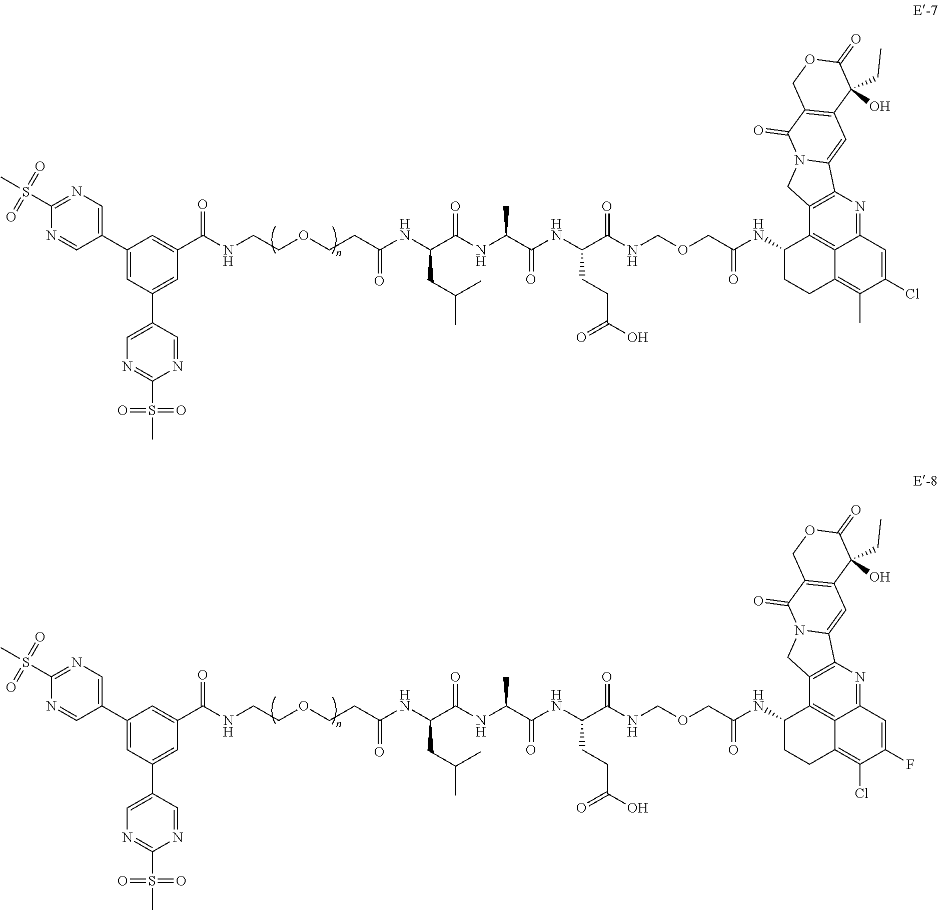 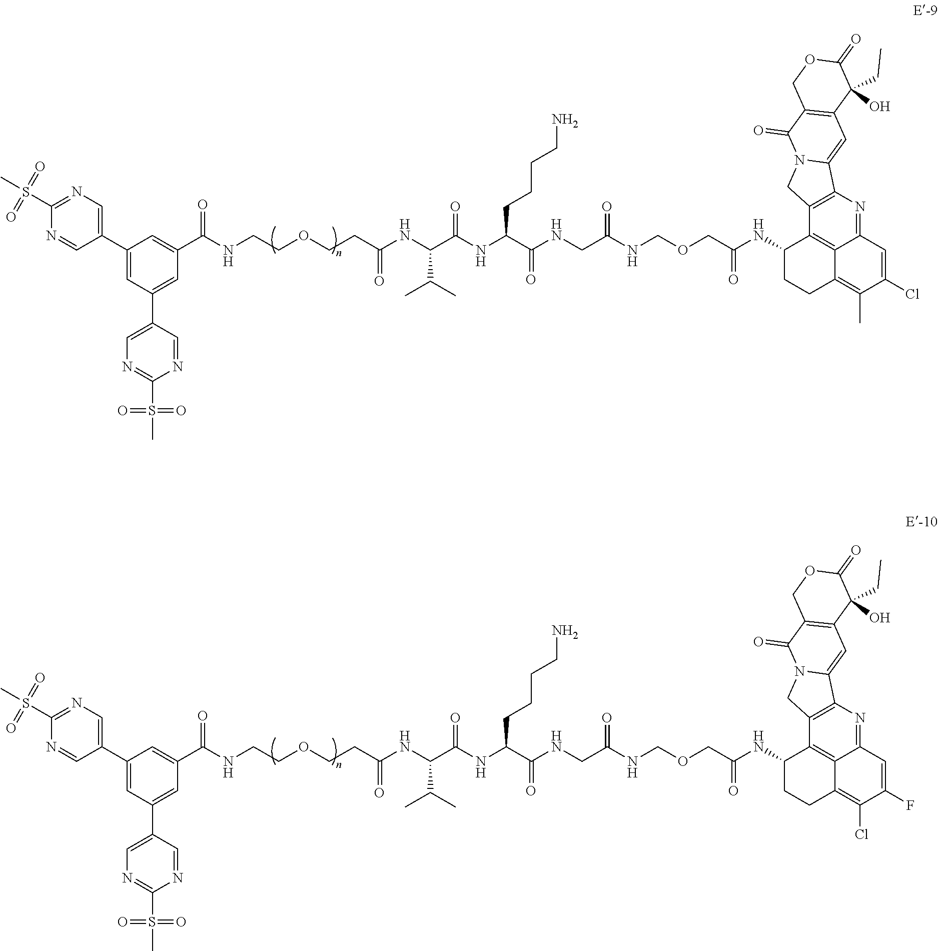 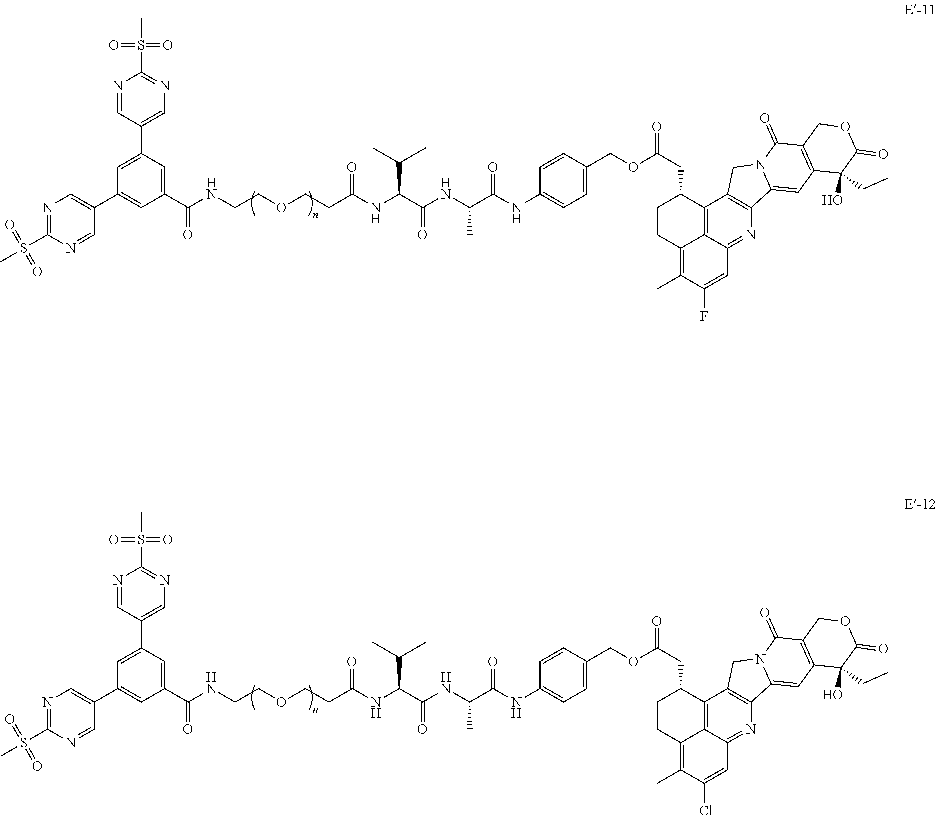 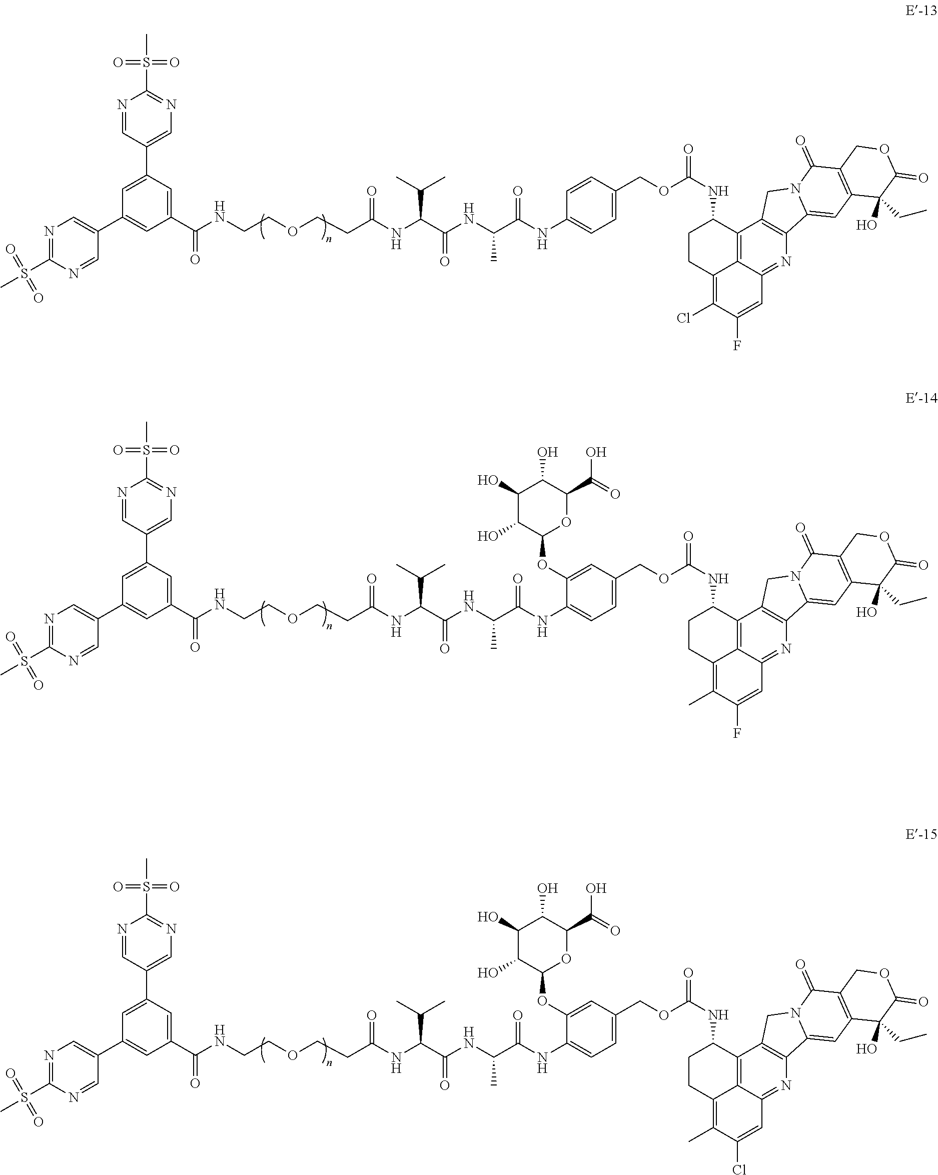 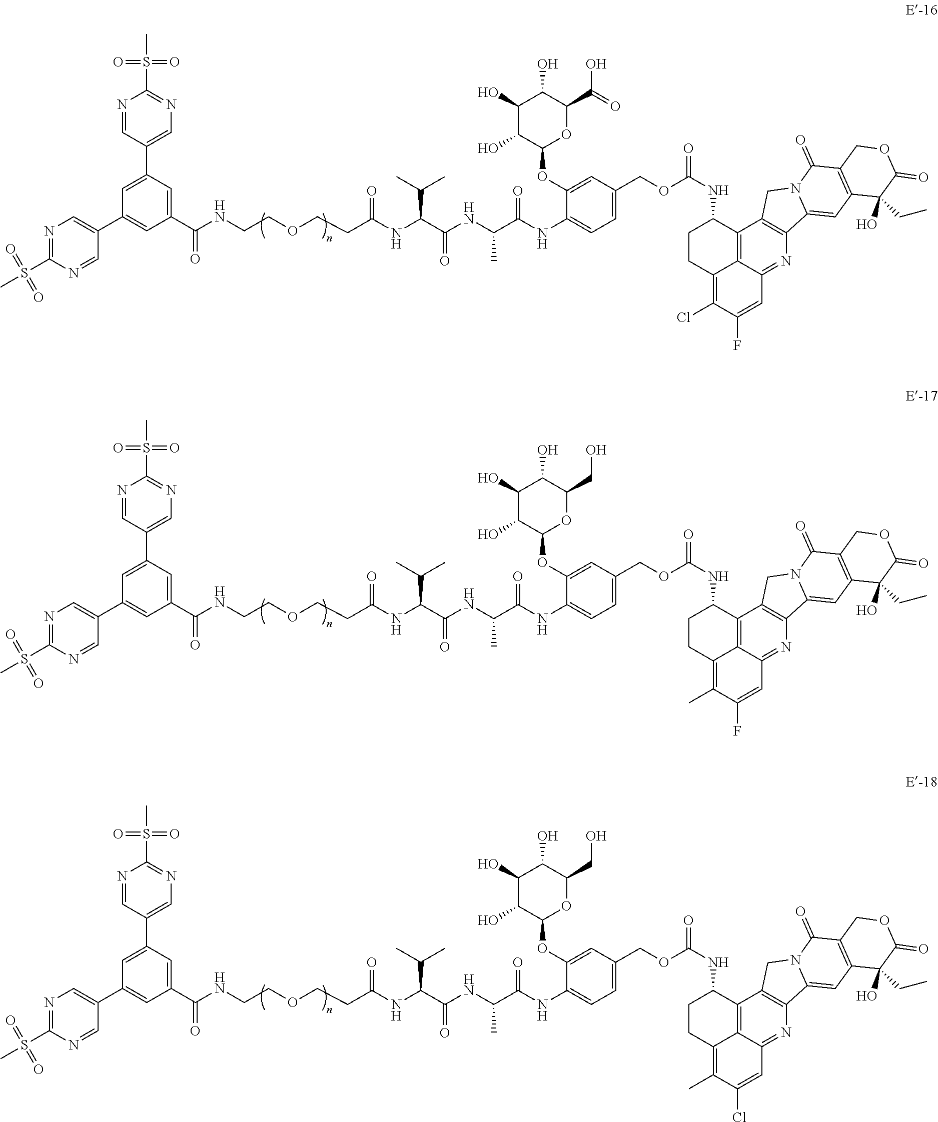 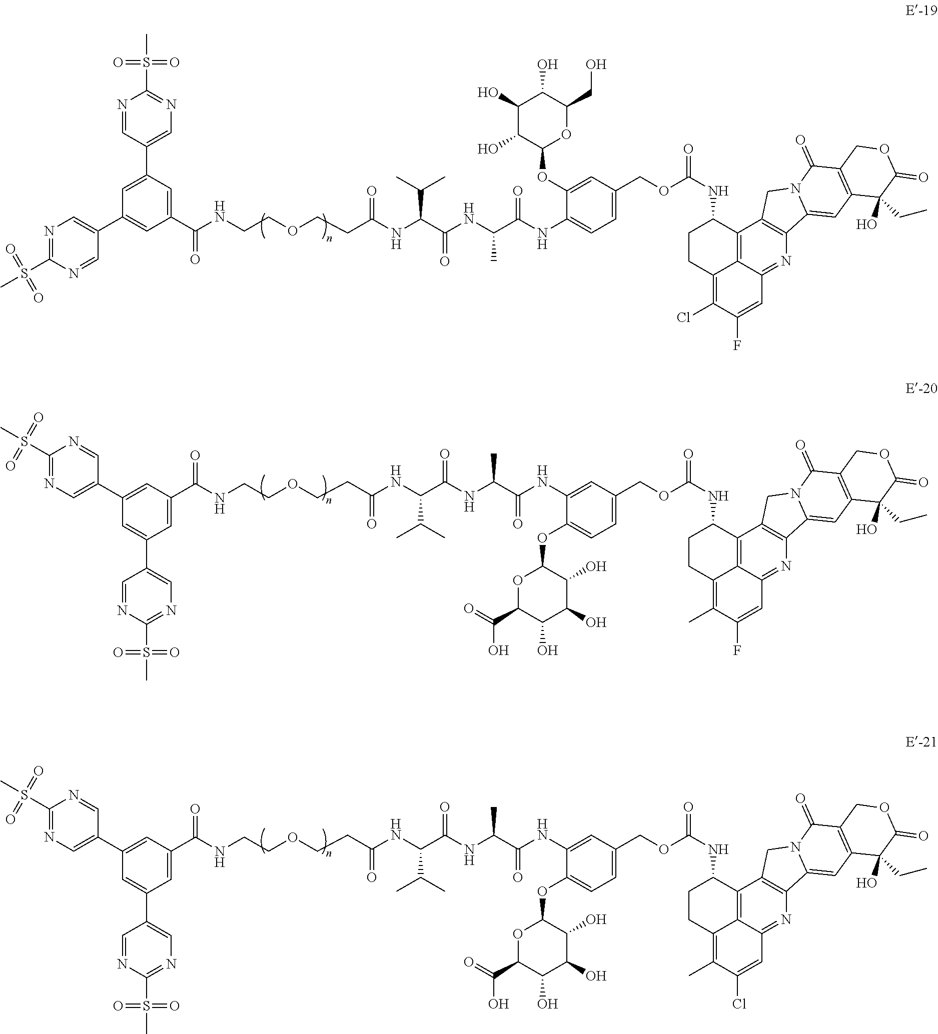 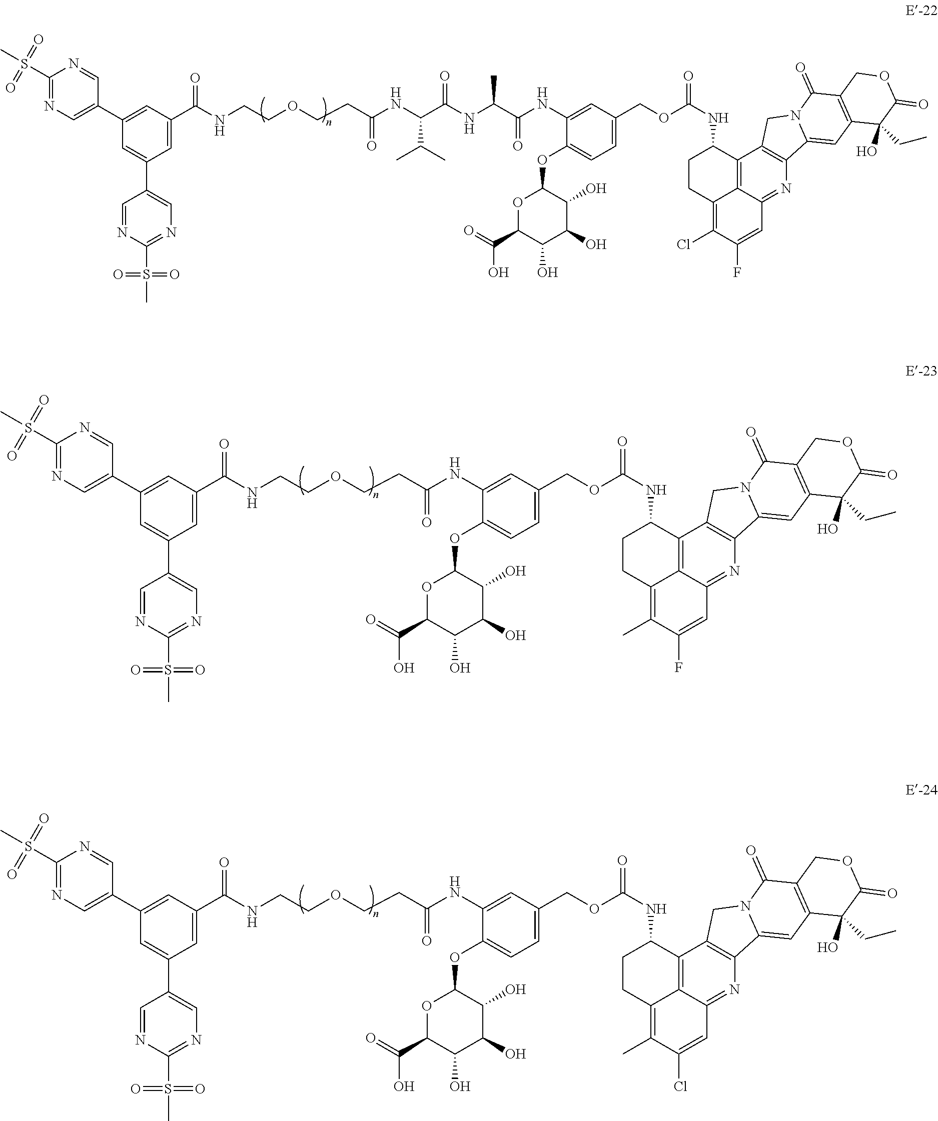 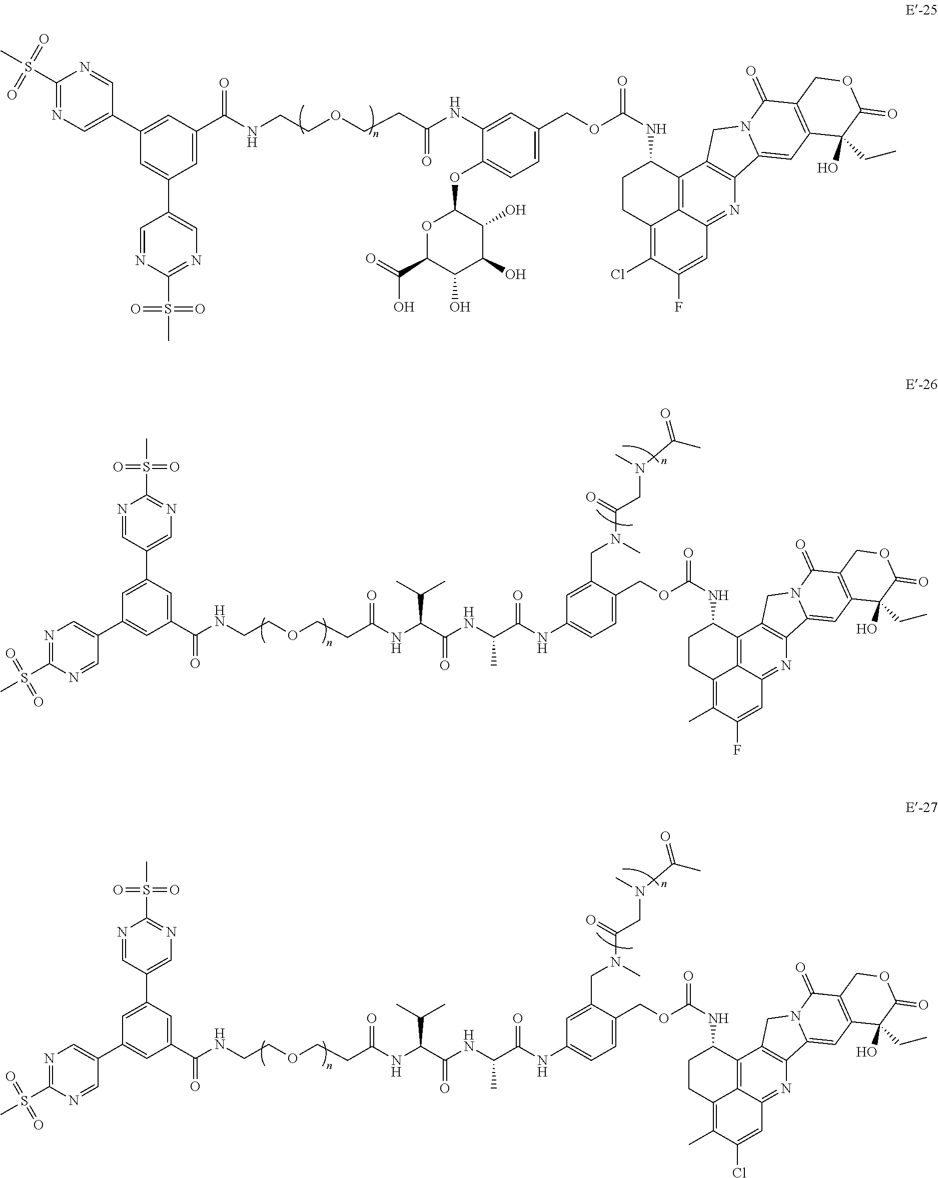 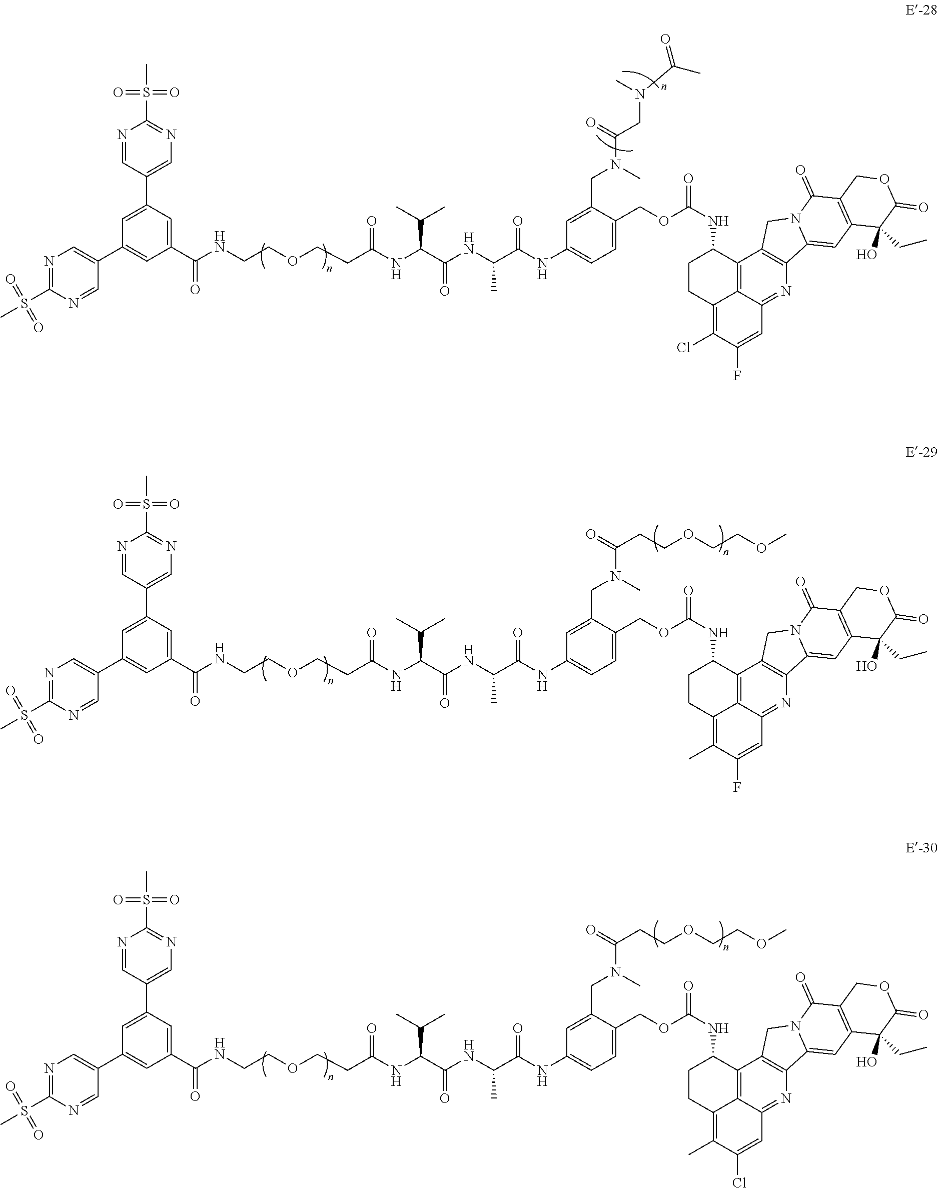 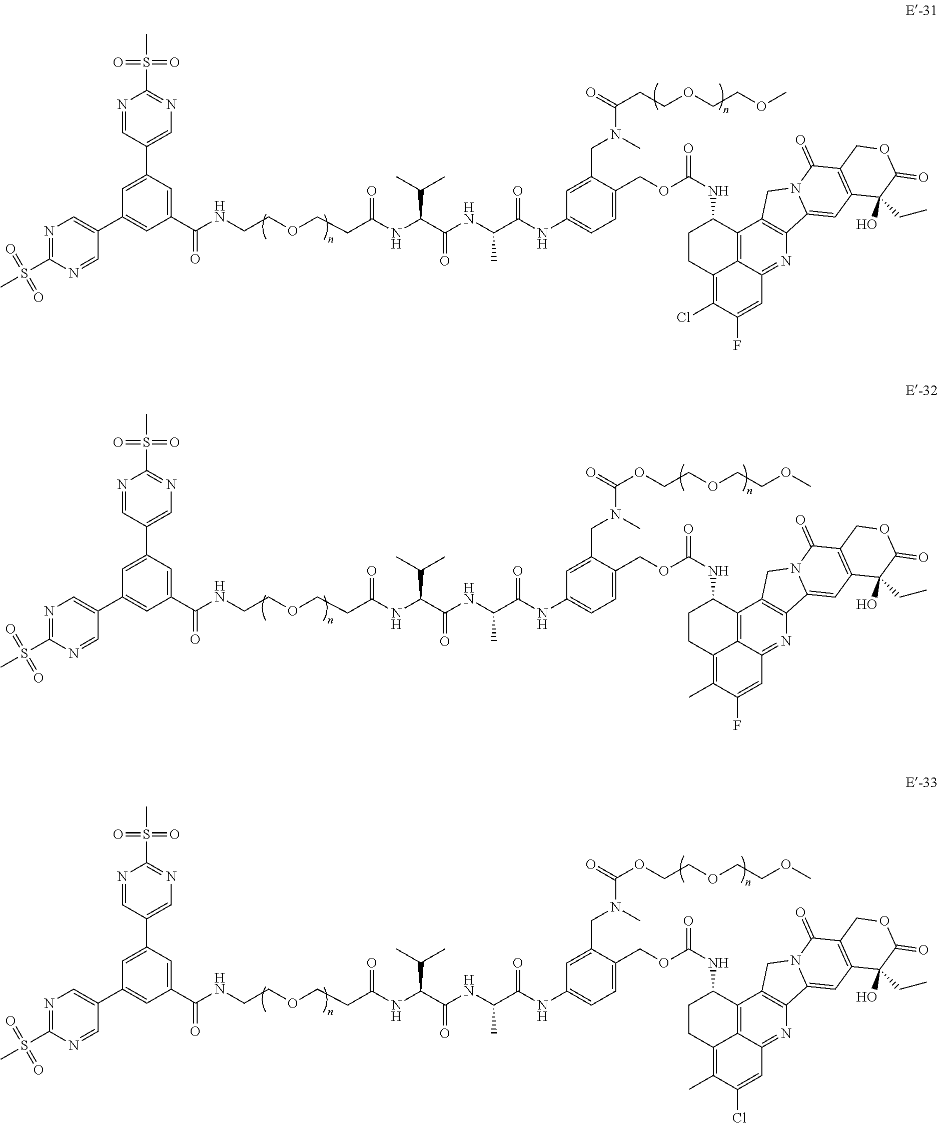 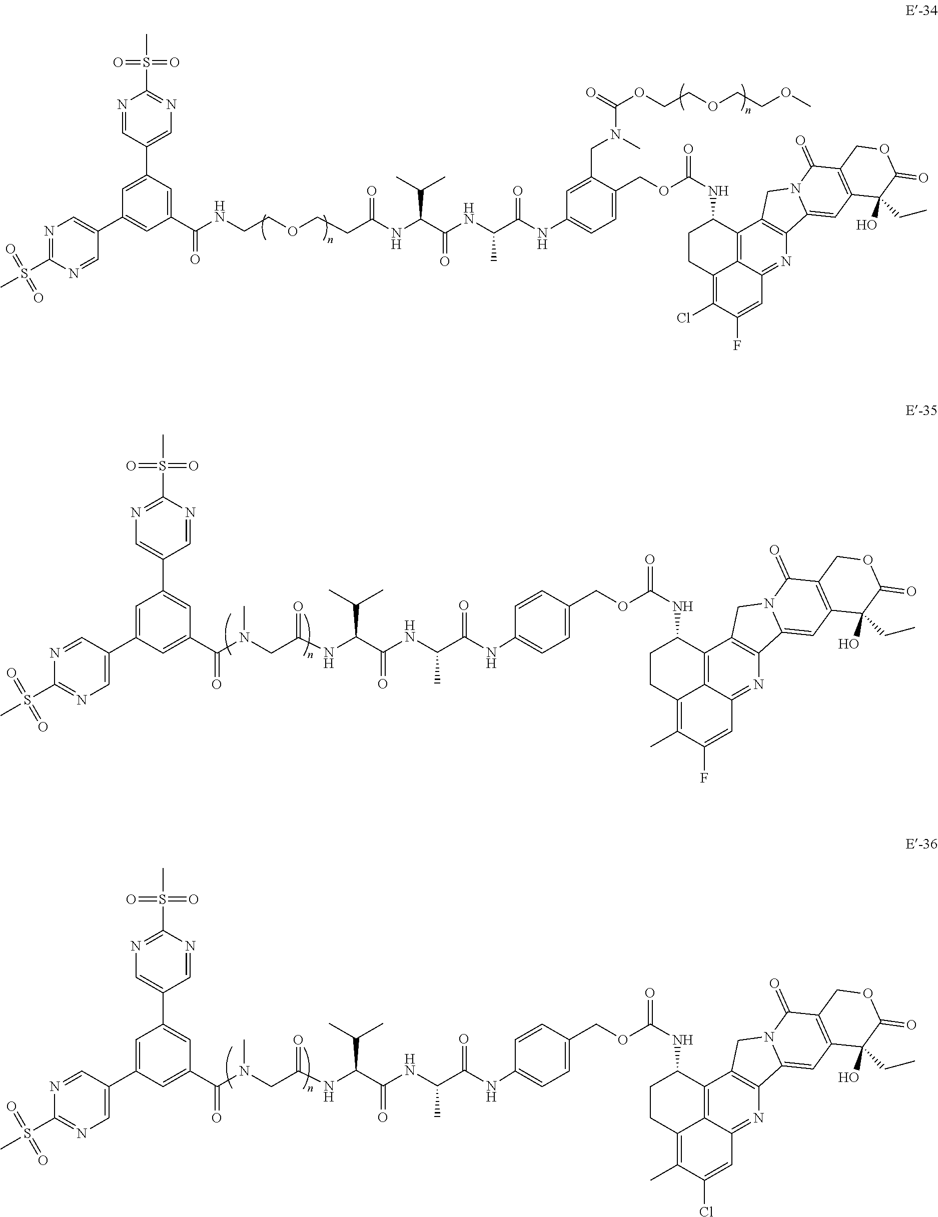 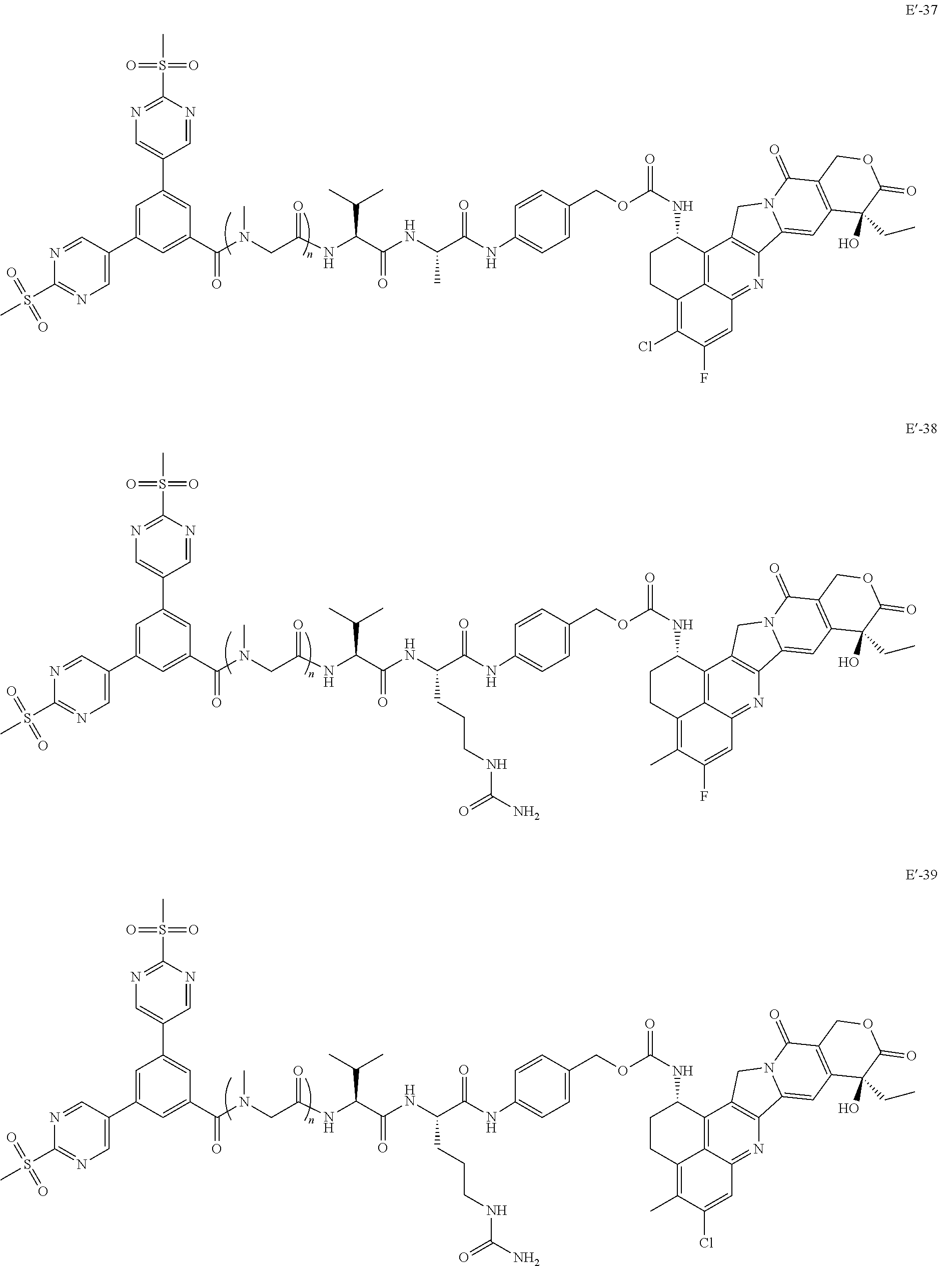 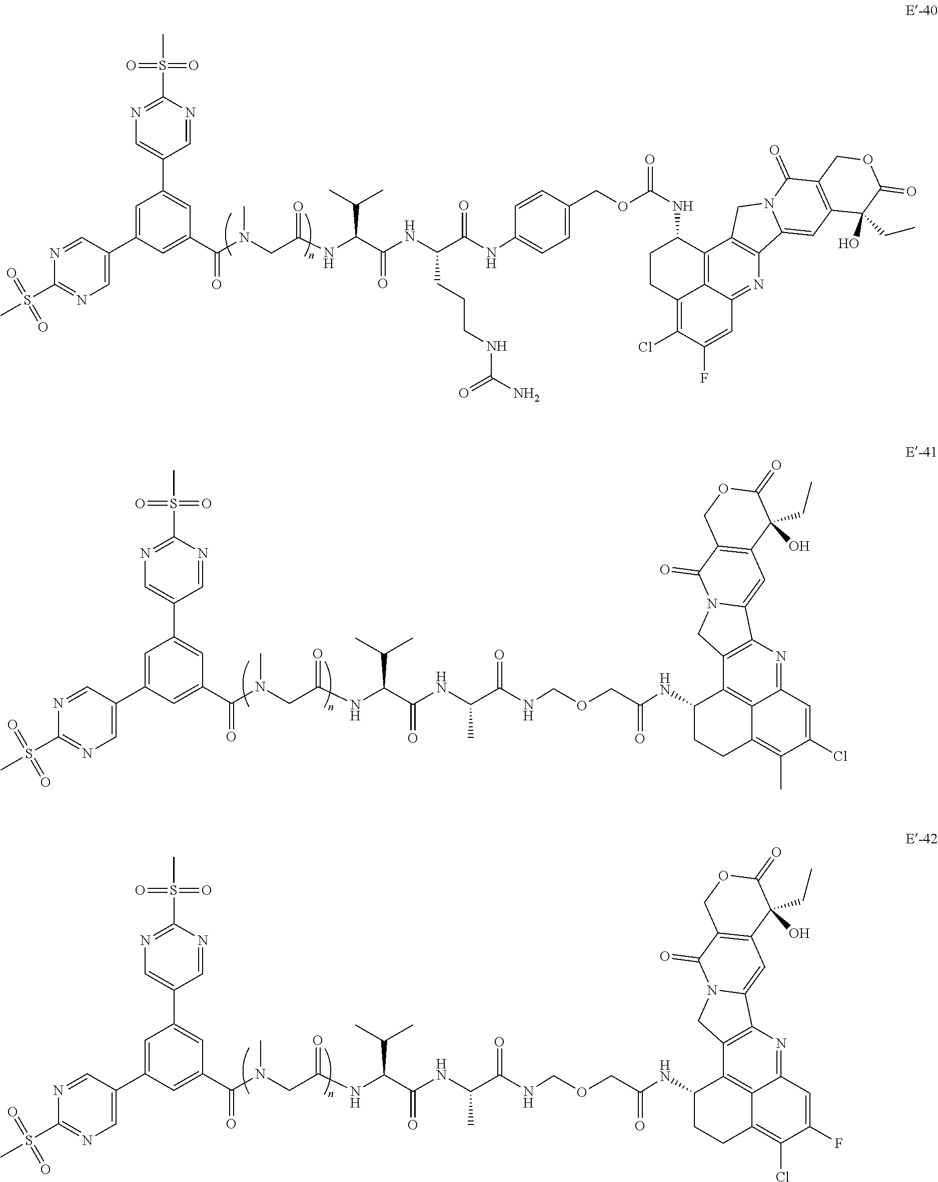 [chemical structure] 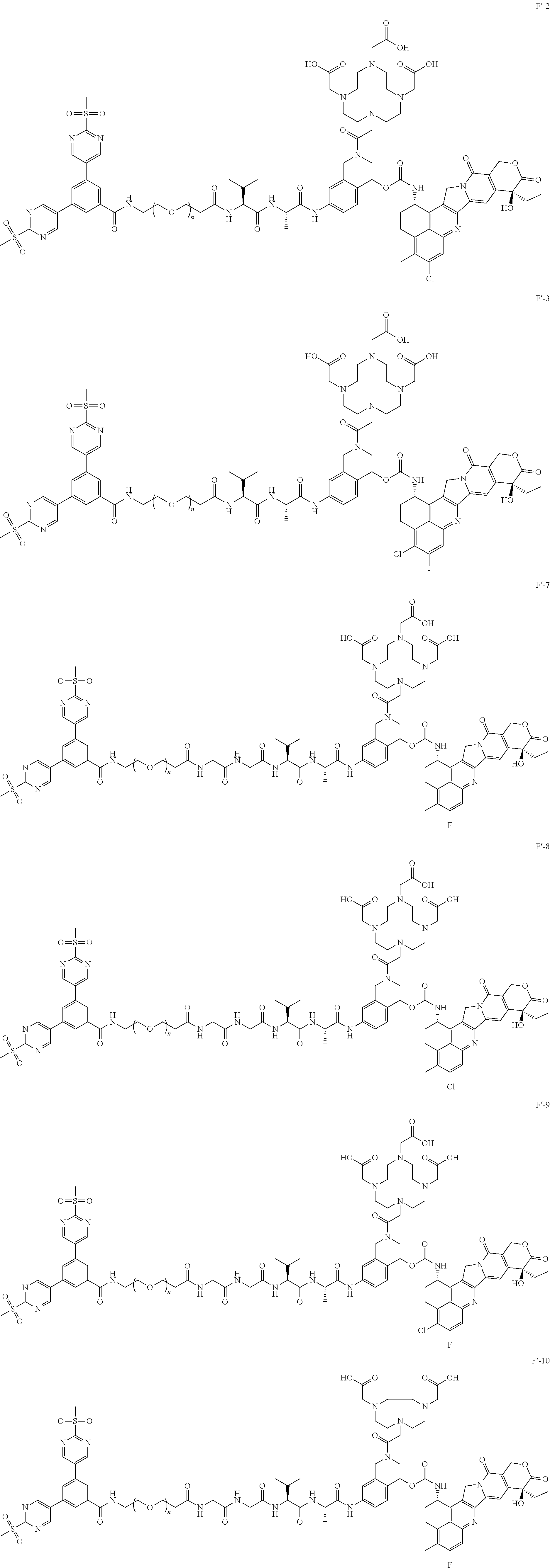 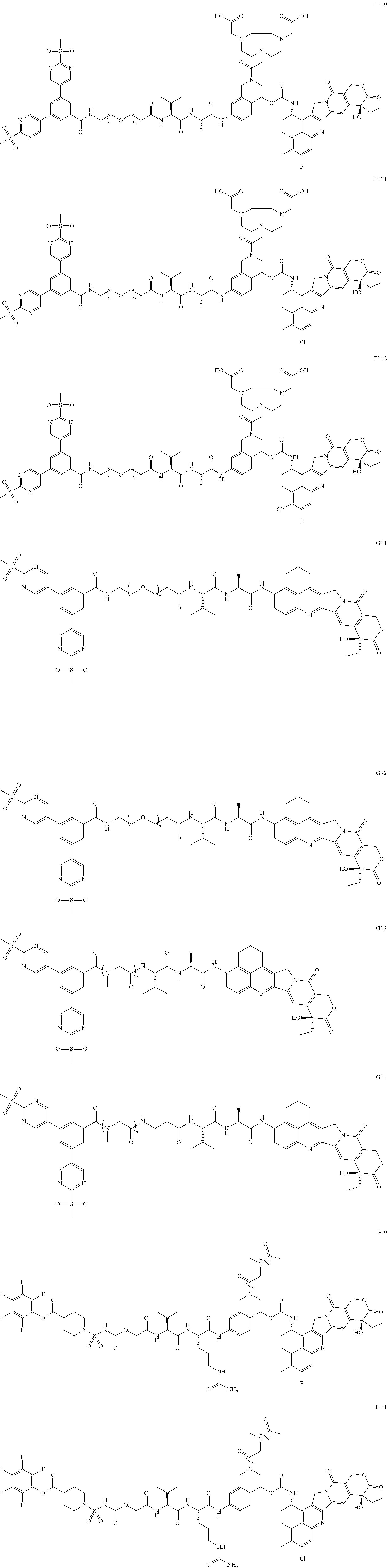 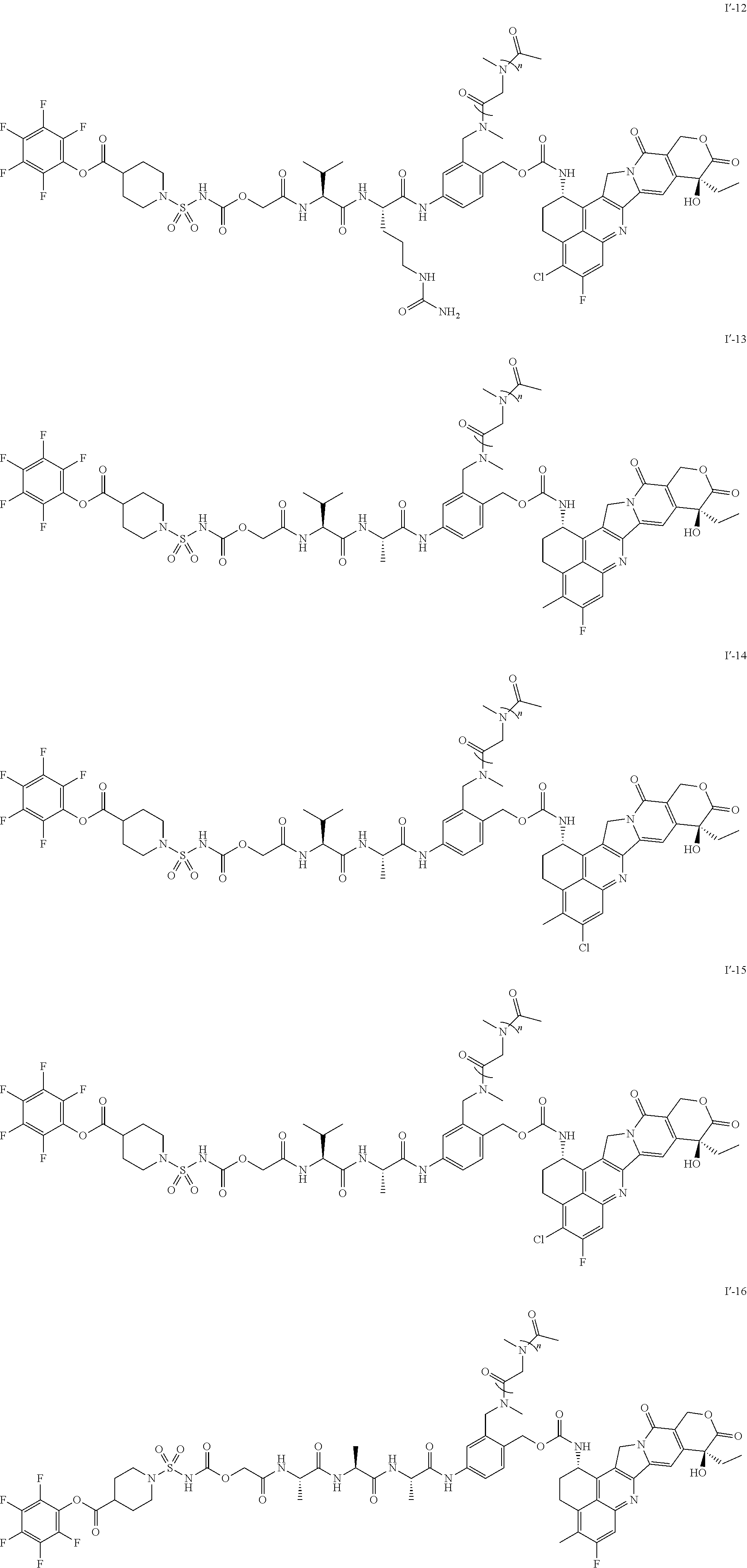 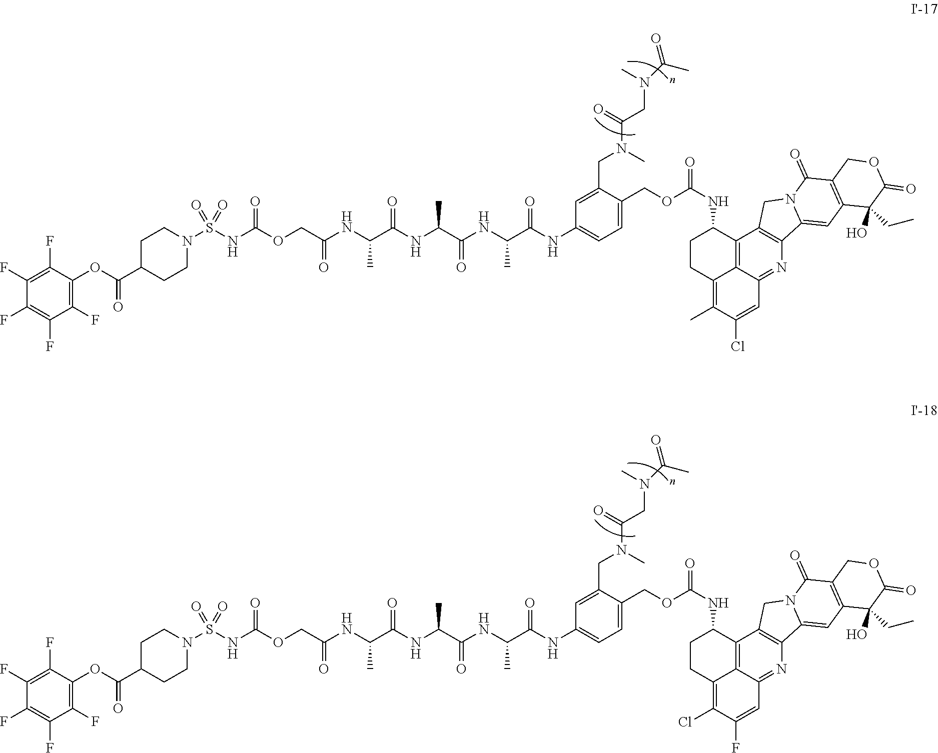 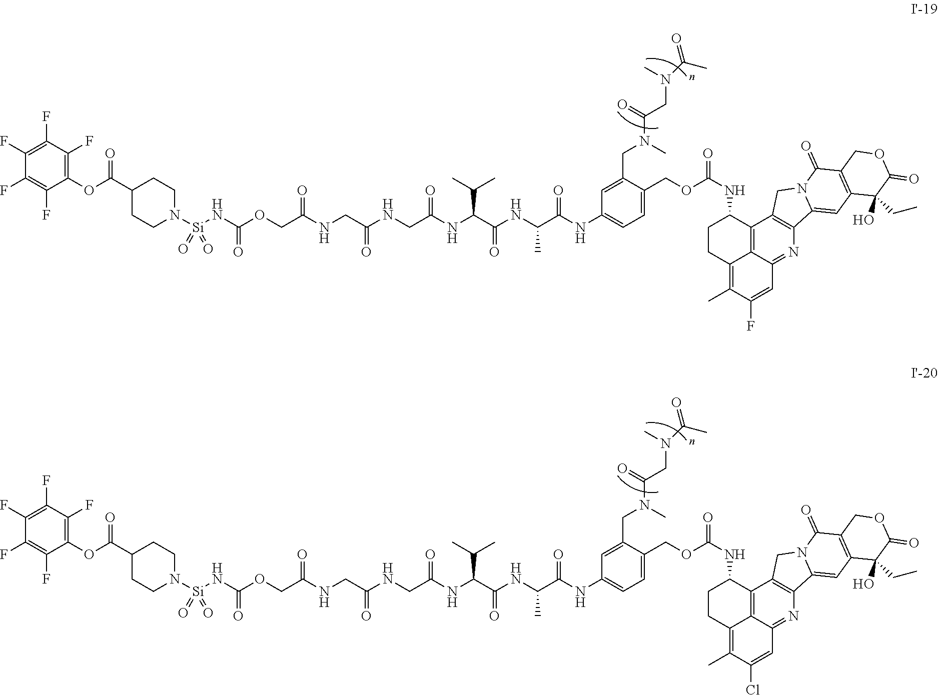 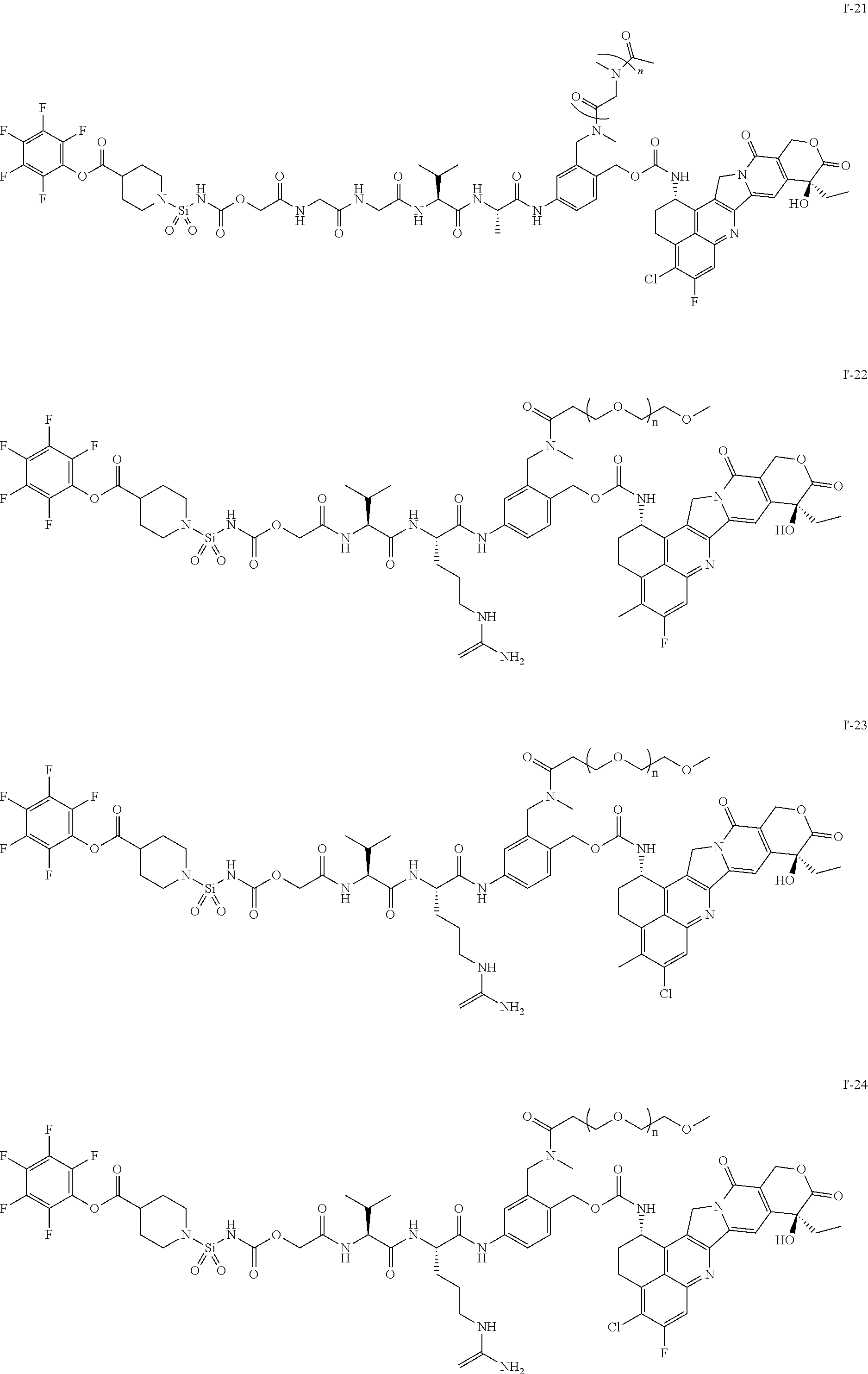 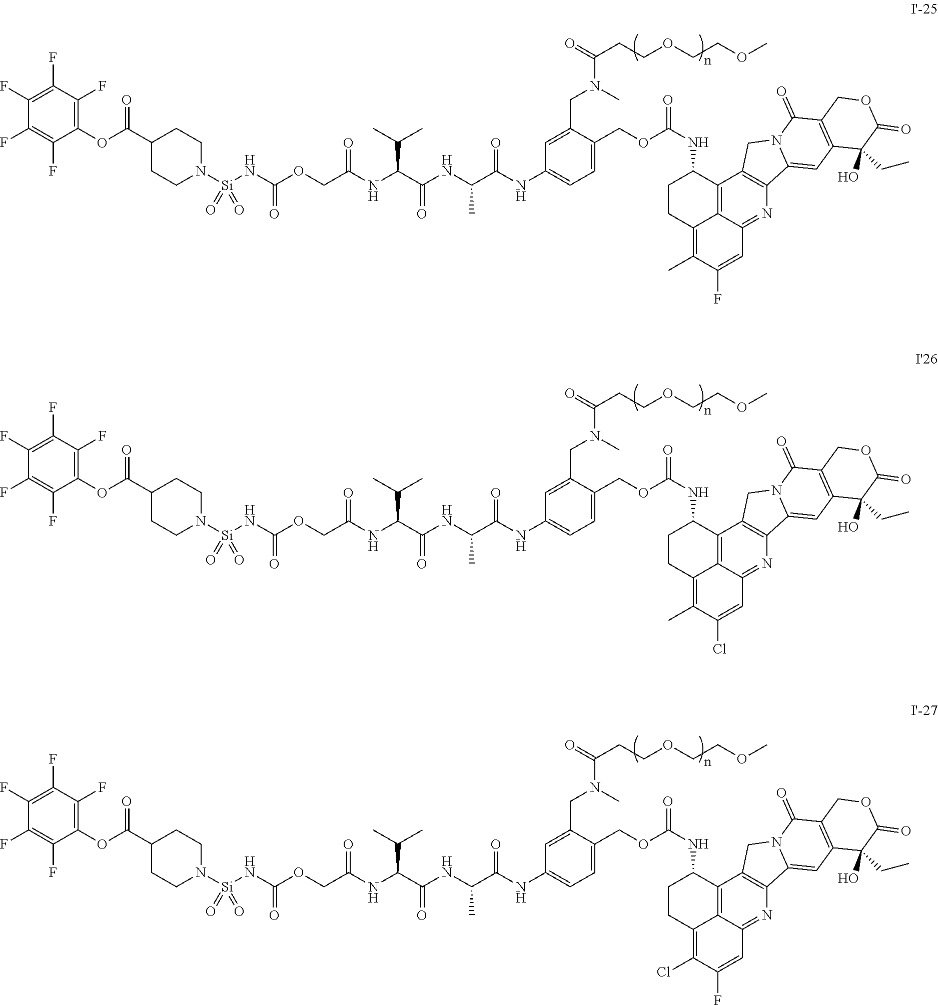 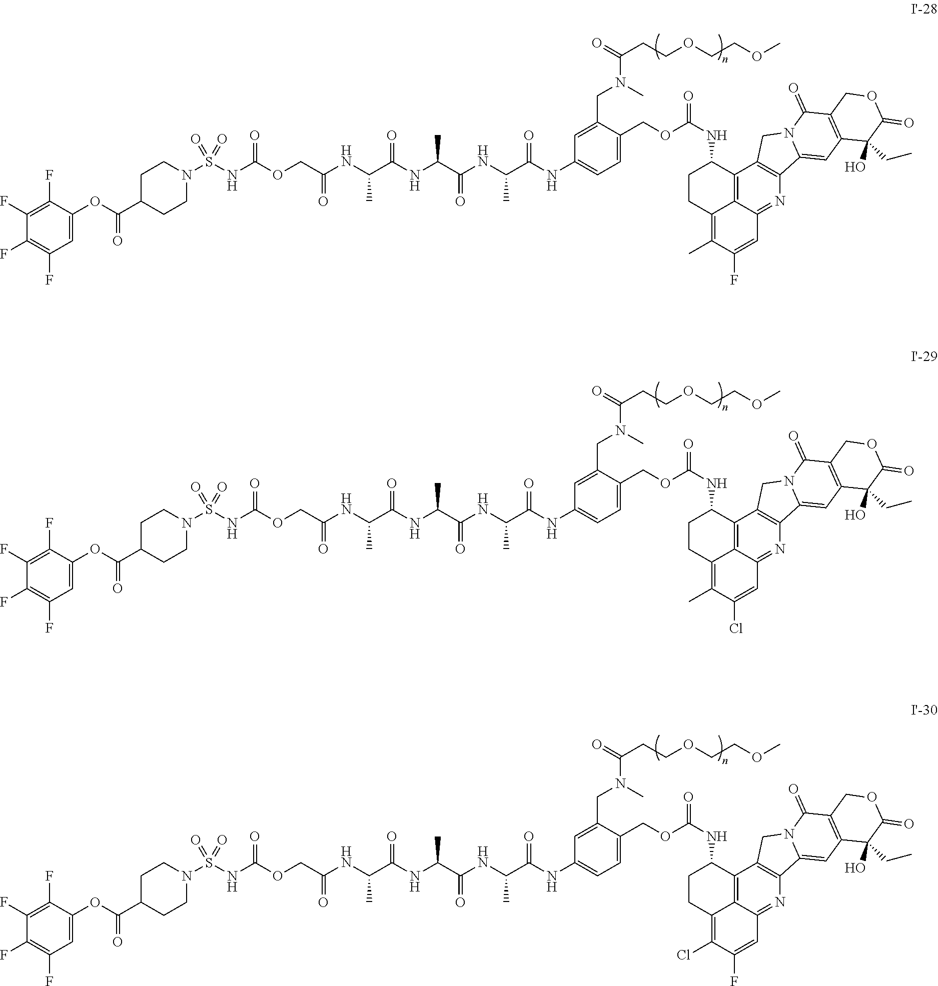 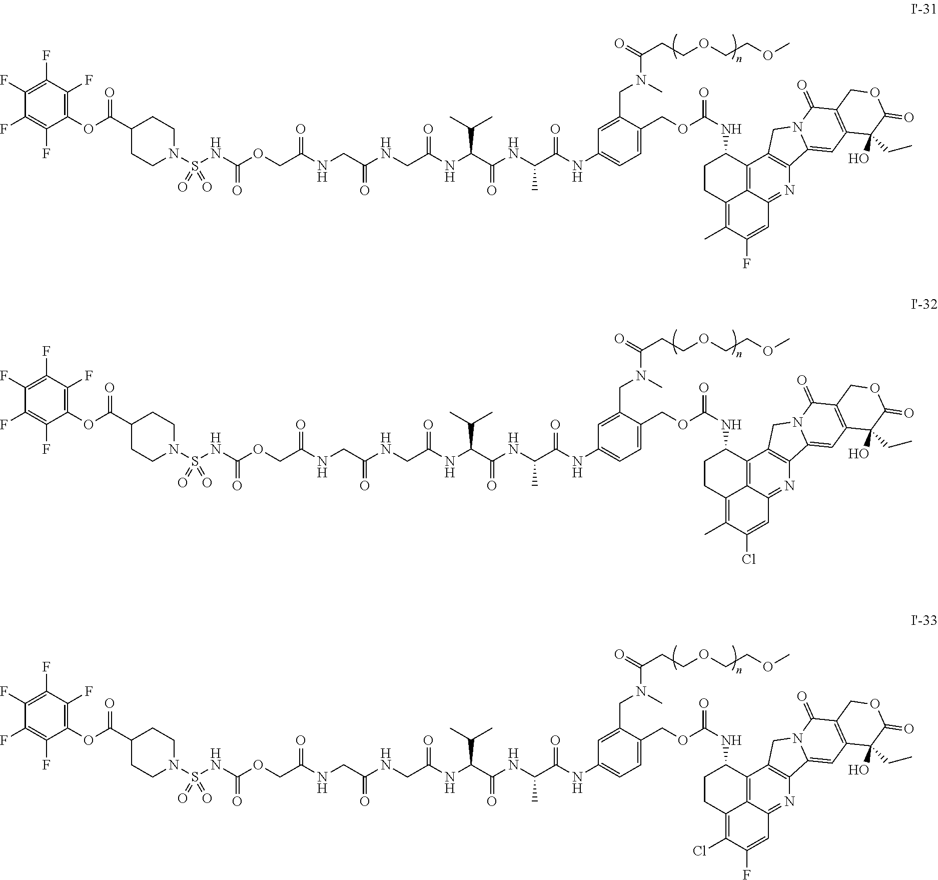 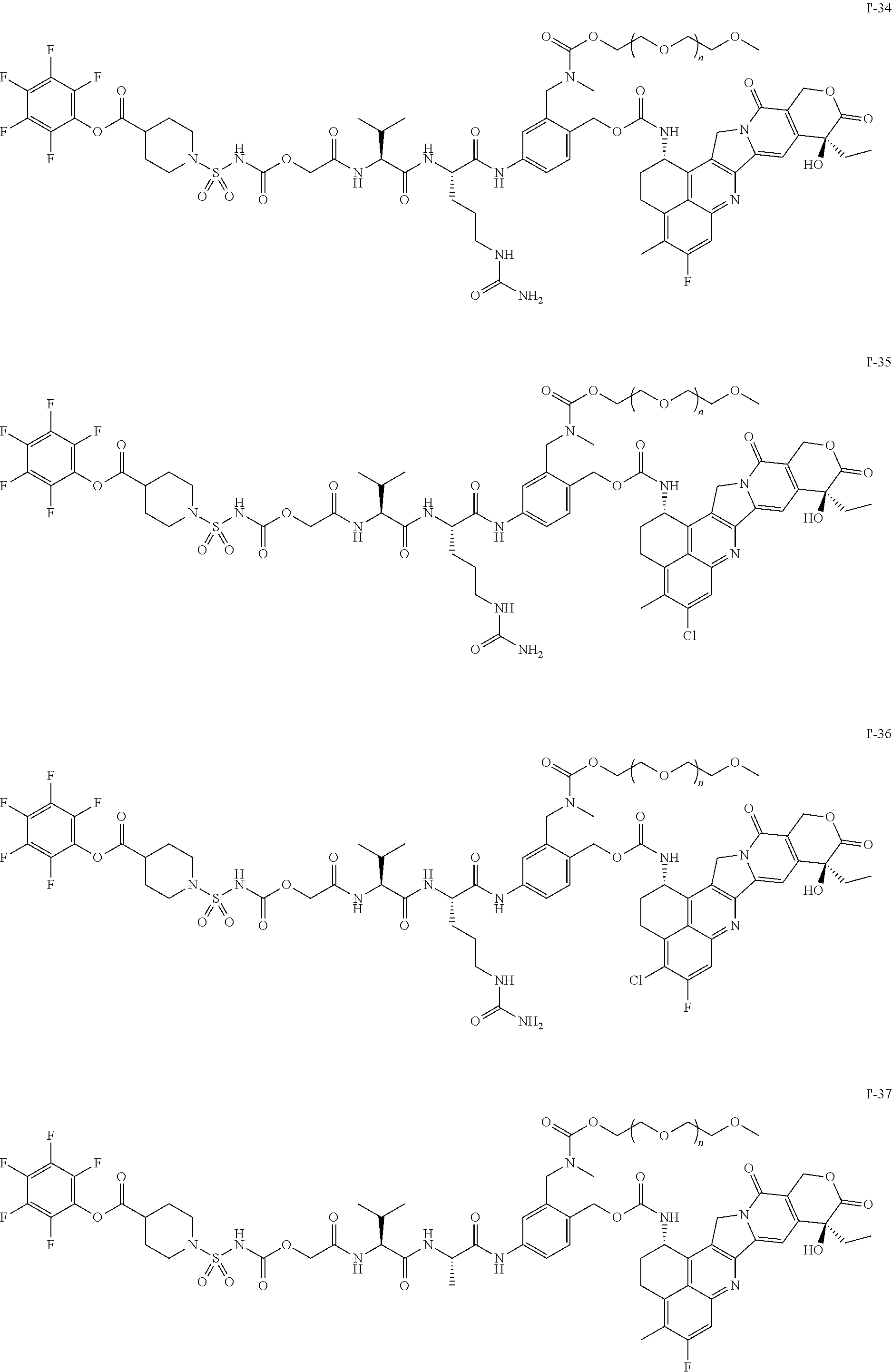 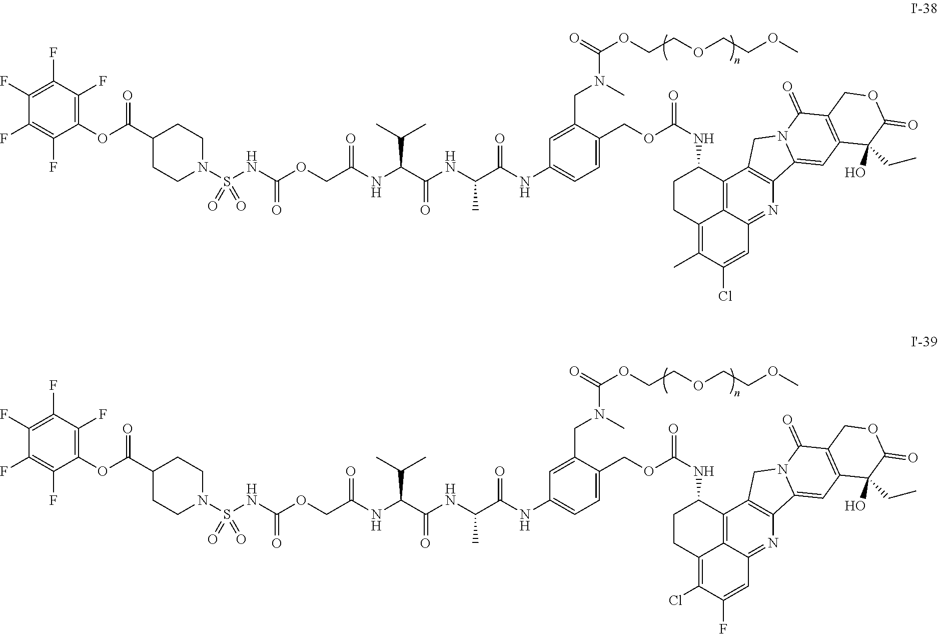 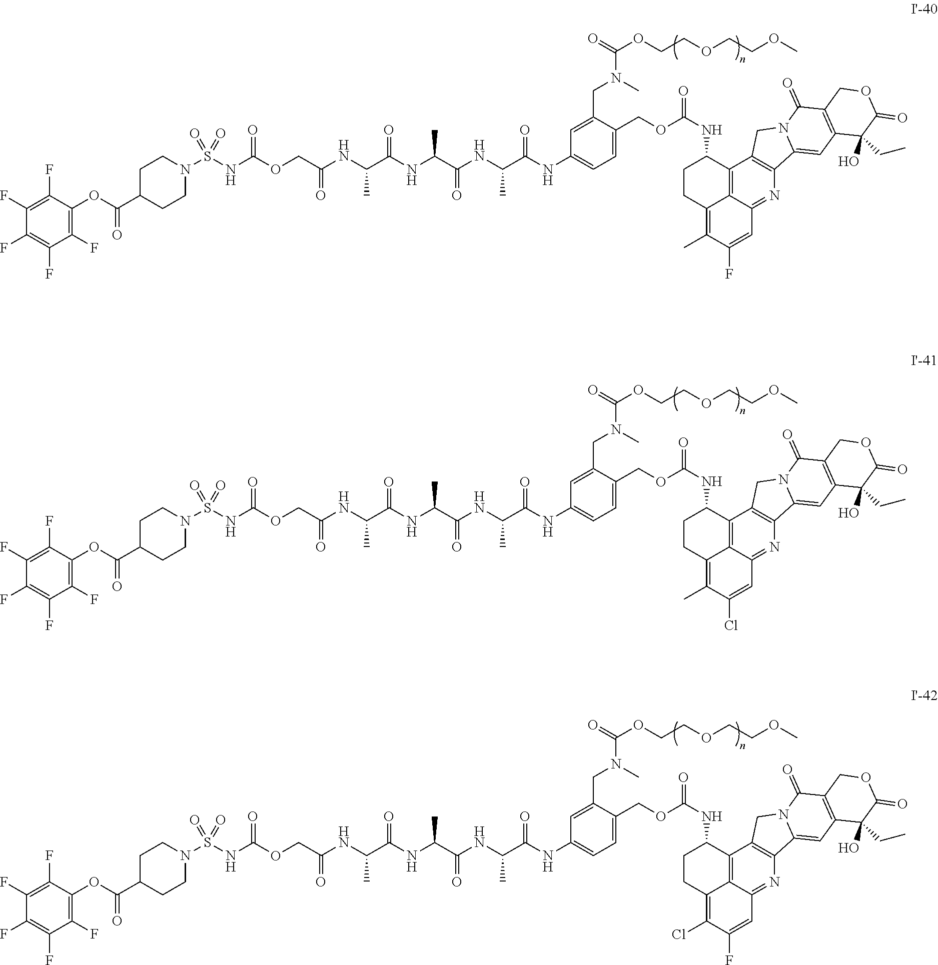 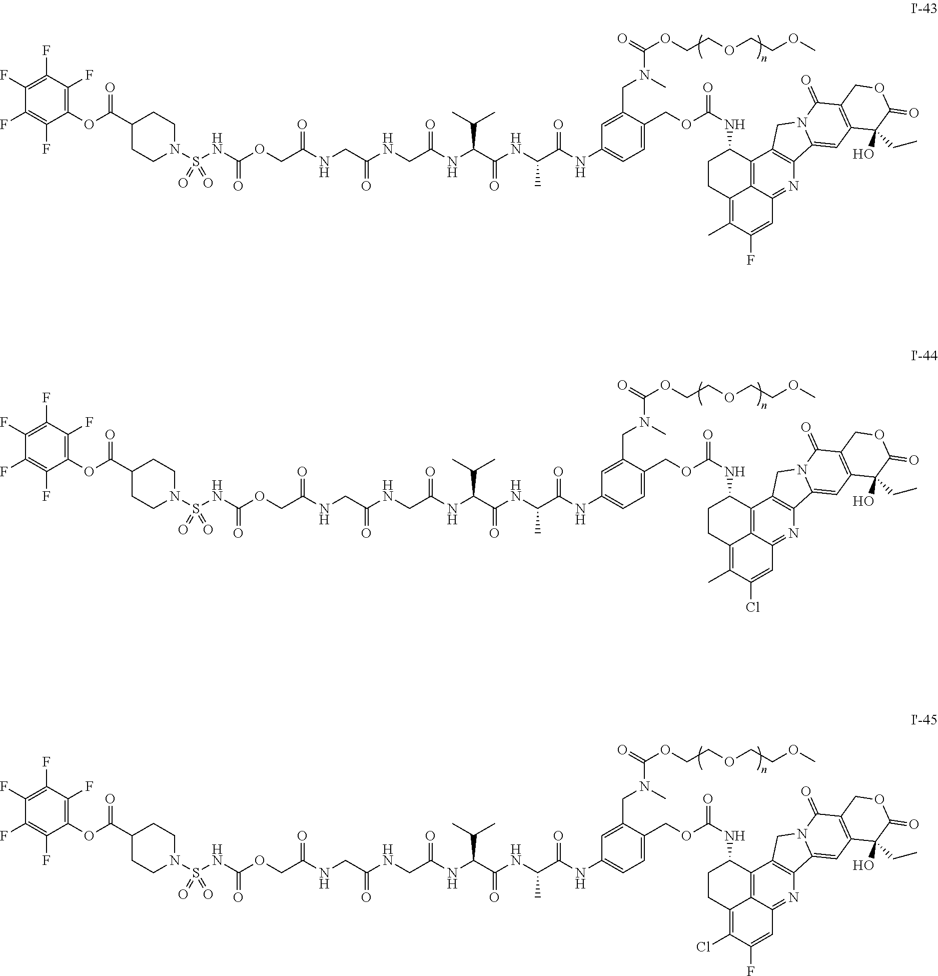 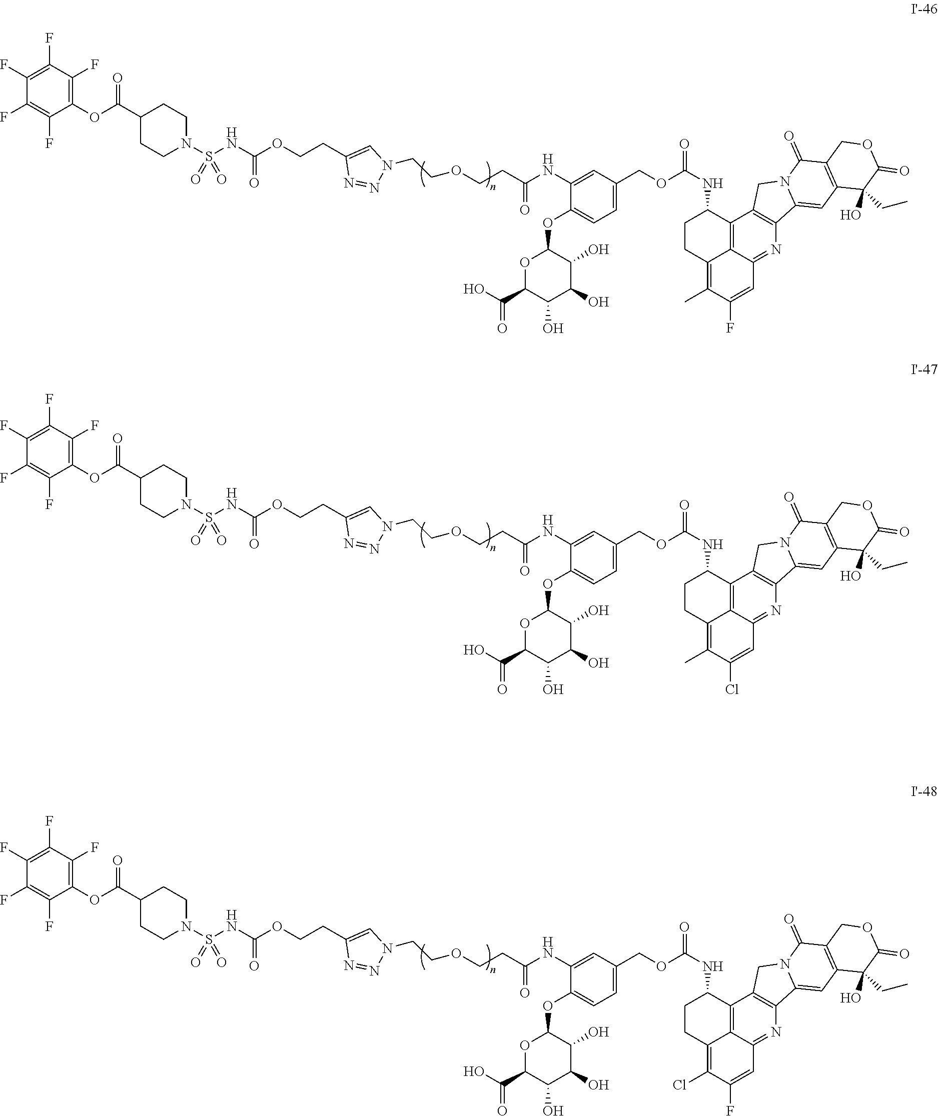 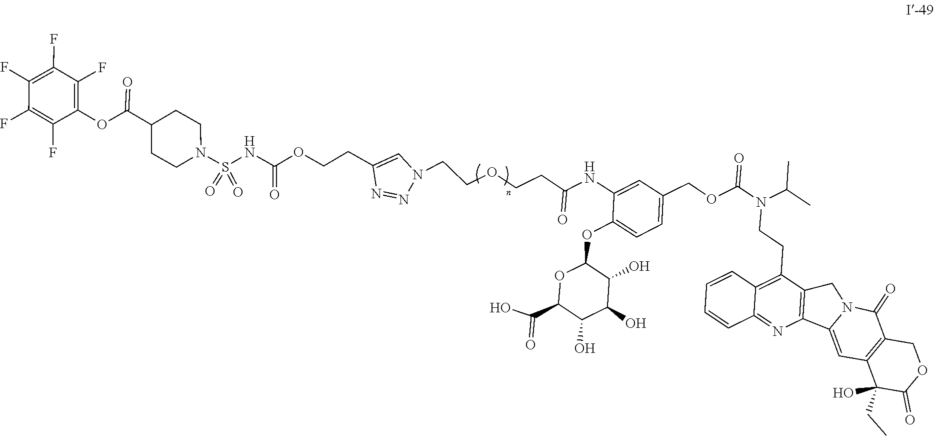
wherein n is an integer selected from 1 to 20, such as an integer of 1 to 12, 3 to 12, for example, n is 1, 2, 3, 4, 5, 6, 7, 8, 9, 10, 11, 12, 13, 14 or 15;
or, -M-L-E-D can be obtained from the following structures: E-1 to E-50, F-1 to F-14, G-1 to G-5 through a substitution reaction (e.g., removal of a methylsulfonyl structure thereon);
or, -M-L-E-D can be obtained from the following structures: E-1 to E-50, F-1 to F-12 through a substitution reaction (e.g., removal of a methylsulfonyl structure thereon);
or, -M-L-E-D can be obtained from the following structures: E-1 to E-50, I-1 to I-49 through a substitution reaction (e.g., removal of a methylsulfonyl structure thereon).

8. The antibody-drug conjugate according to claim 1, wherein the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following 3 CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(1b) a heavy chain variable region (VH) comprising the following 3 CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO:22 or variant thereof; and/or, a light chain variable region (VL) comprising the following 3 CDRs: a CDR-L1 having a sequence as set forth in SEQ ID NO:23 or variant thereof, a CDR-L2 having a sequence as set forth in SEQ ID NO:24 or variant thereof, a CDR-L3 having a sequence as set forth in SEQ ID NO:25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following 3 CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(2b) a heavy chain variable region (VH) comprising the following 3 CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO. NO:: 22 or variant thereof; and/or, a light chain variable region (VL) comprising the following 3 CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition one or more amino acids (e.g., a substitution, deletion or addition 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following 3 CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(3b) a heavy chain variable region (VH) comprising the following 3 CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO. NO:: 22 or variant thereof; and/or, a light chain variable region (VL) comprising the following 3 CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a) and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL), wherein the CDRs are defined according to the IMGT numbering system:
(4a) a heavy chain variable region (VH) comprising the following 3 CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof; and/or, a light chain variable region (VL) comprising the following 3 CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof; or,
(4b) a heavy chain variable region (VH) comprising the following 3 CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO. NO:: 30 or variant thereof; and/or, a light chain variable region (VL) comprising the following 3 CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
preferably, the antibody or antigen-binding fragment thereof comprises:
(1) the following heavy chain variable region (VH) and light chain variable region (VL), wherein the CDRs are defined according to the Chothia numbering system:
(1a) a heavy chain variable region (VH) comprising the following 3 CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following 3 CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and, a light chain variable region (VL) comprising the following 3 CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL), wherein the CDRs are defined according to the AbM numbering system:
(2a) a heavy chain variable region (VH) comprising the following 3 CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following 3 CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and, a light chain variable region (VL) comprising the following 3 CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and light chain variable region (VL), wherein the CDRs are defined according to the Kabat numbering system:
(3a) a heavy chain variable region (VH) comprising the following 3 CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following 3 CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and, a light chain variable region (VL) comprising the following 3 CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and light chain variable region (VL), wherein the CDRs are defined according to the IMGT numbering system:
(4a) a heavy chain variable region (VH) comprising the following 3 CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and, a light chain variable region (VL) comprising the following 3 CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following 3 CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and, a light chain variable region (VL) comprising the following 3 CDRs: a CDR-L1 with a sequence as set forth in SEQ ID NO: 31, a CDR-L2 with a sequence as set forth in SEQ ID NO: 32, and a CDR-L3 with a sequence as set forth in SEQ ID NO: 25.

9. The antibody-drug conjugate according to claim 1, wherein the antibody or antigen-binding fragment thereof comprises:
(a) a VH as set forth in SEQ ID NO: 1 or variant thereof, and/or, a VL as set forth in SEQ ID NO: 2 or variant thereof, or
(b) a VH as set forth in SEQ ID NO: 3 or variant thereof, and/or, a VL as set forth in SEQ ID NO: 4 or variant thereof;
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution;
preferably, the antibody or antigen-binding fragment thereof comprises:
(a) a VH as set forth in SEQ ID NO: 1, and, a VL as set forth in SEQ ID NO: 2; or (b) a VH as set forth in SEQ ID NO: 3, and a VL as set forth in SEQ ID NO: 4.

10. The antibody-drug conjugate according to claim 8, wherein the antibody or antigen-binding fragment thereof further comprises:
(a) a human immunoglobulin heavy chain constant region (CH) or variant thereof, wherein the variant has a substitution, deletion or addition of one or more amino acids (e.g., a substitution, deletion or addition of up to 20, up to 15, up to 10, or up to 5 amino acids; e.g., a substitution, deletion or addition of 1, 2, 3, 4 or 5 amino acids) as compared with the wild-type sequence from which it is derived; and
(b) a human immunoglobulin light chain constant region (CL) or variant thereof, wherein the variant has a substitution, deletion or addition of one or more amino acids (e.g., a substitution, deletion or addition of up to 20, up to 15, up to 10, or up to 5 amino acids; e.g., a substitution, deletion or addition of 1, 2, 3, 4 or 5 amino acids) as compared with the wild-type sequence from which it is derived;
preferably, the heavy chain constant region is an IgG heavy chain constant region, such as an IgG1, IgG2, IgG3 or IgG4 heavy chain constant region, such as a human IgG1 heavy chain constant region or a human IgG4 heavy chain constant region;
preferably, the antibody or antigen-binding fragment thereof comprises a heavy chain constant region (CH) as set forth in SEQ ID NO: 35 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 35;
preferably, the antibody or antigen-binding fragment thereof comprises a light chain constant region (CL) as set forth in SEQ ID NO: 36 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 36;
preferably, the antibody or antigen-binding fragment thereof comprises a heavy chain constant region (CH) as set forth in SEQ ID NO: 35 and a light chain constant region (CL) as set forth in SEQ ID NO: 36.

11. The antibody-drug conjugate according to claim 1, wherein the antibody or antigen-binding fragment thereof comprises:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36;
optionally, the VH having a sequence as set forth in SEQ ID NO: 1 or 3 or variant thereof or the heavy chain having a sequence as set forth in SEQ ID NO: 37 or 39 or variant thereof undergoes cyclization to form pyroglutamic acid or pyroglutamate;
optionally, the heavy chain constant region (CH) as set forth in SEQ ID NO: 35 or variant thereof or the heavy chain having a sequence as set forth in SEQ ID NO: 37 or 39 or variant thereof lacks C-terminal lysine.

12. The antibody-drug conjugate according to claim 1, M is connected to the sulfhydryl group (—SH) or amino group (—NH₂) on Ab;
preferably, M is connected to the sulfhydryl group (—SH) on Ab.

13. The antibody-drug conjugate according to claim 1, wherein the antibody or antigen-binding fragment thereof is selected from the group consisting of the antibody or antigen-binding fragment thereof comprises:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence a set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36;
optionally the VH having a sequence as set forth in SEQ ID NO: 1 or 3 or variant thereof or the heavy chain having a sequence as set forth in SEQ ID NO: 37 or 39 or variant thereof undergoes cyclization to form pyroglutamic acid or pyroglutamate;
optionally, the heavy chain constant region (CH) as set forth in SEQ ID NO: 35 or variant thereof or the heavy chain having a sequence as set forth in SEQ ID NO: 37 or 39 or variant thereof lacks C-terminal lysine;
-M-L-E-D is selected from the group consisting of the structure formed from E′-1 to E′-50, F′-1 to F′-3, F′-7 to F′-12, G′-1 to G′-4, P′-10 to P′-49, E-1 to E-50, F-1 to F-14, G-1 to G-26, I-1 to I-50, 1 example, via a substitution reaction (e.g., through the removal of the methylsulfonyl group thereon);
x is 1 to 10; preferably, x is 1 to 6, or x is 3 to 6, and further preferably, x is about 2, or x is about 4.

14. The antibody-drug conjugate according to claim 8, which is selected from the group consisting of: 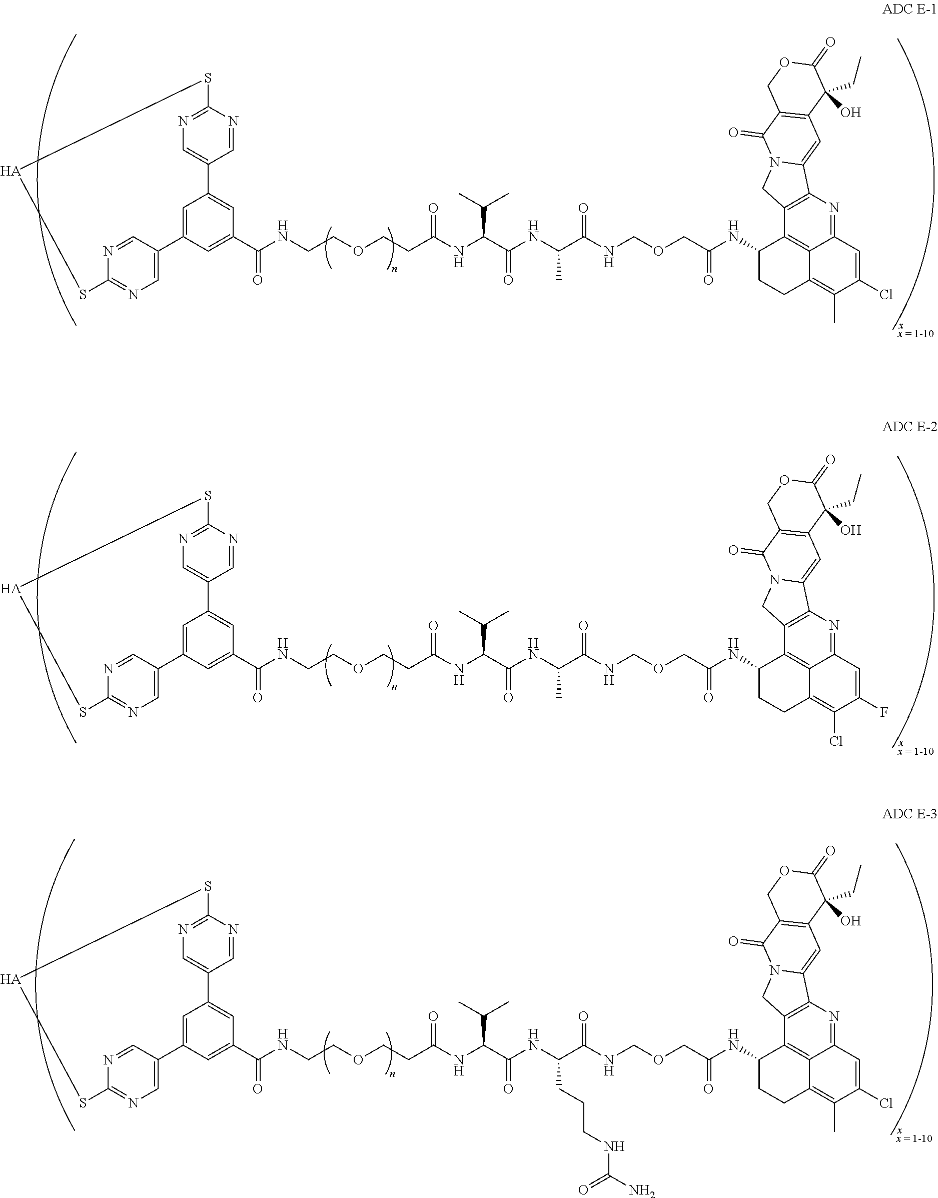 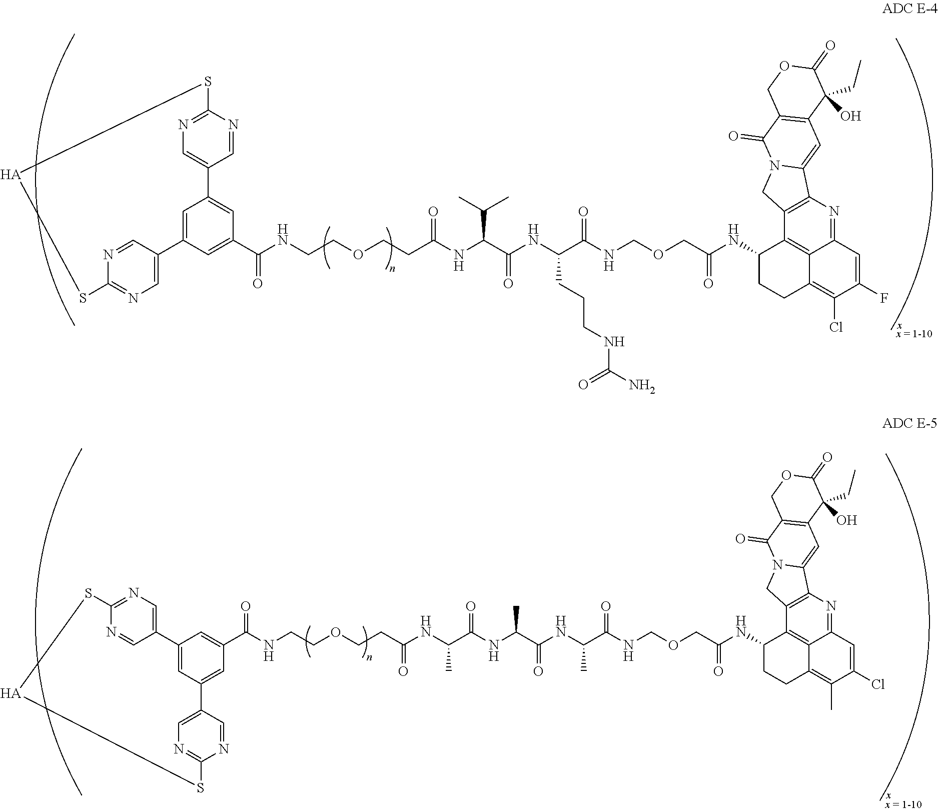 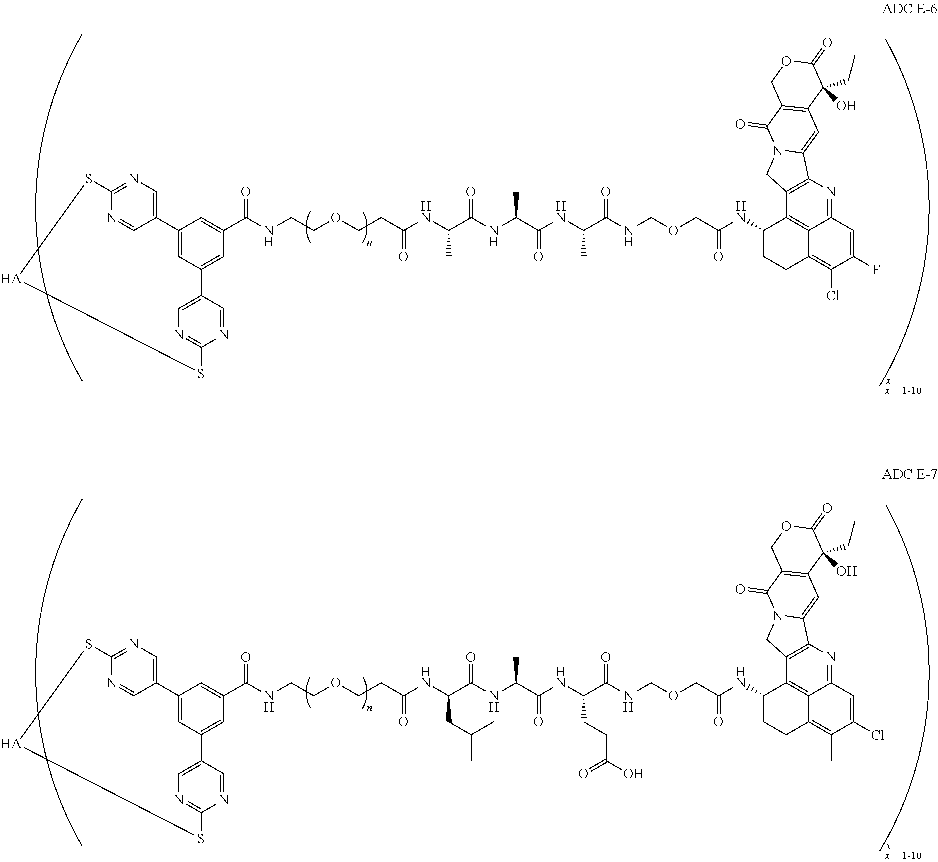 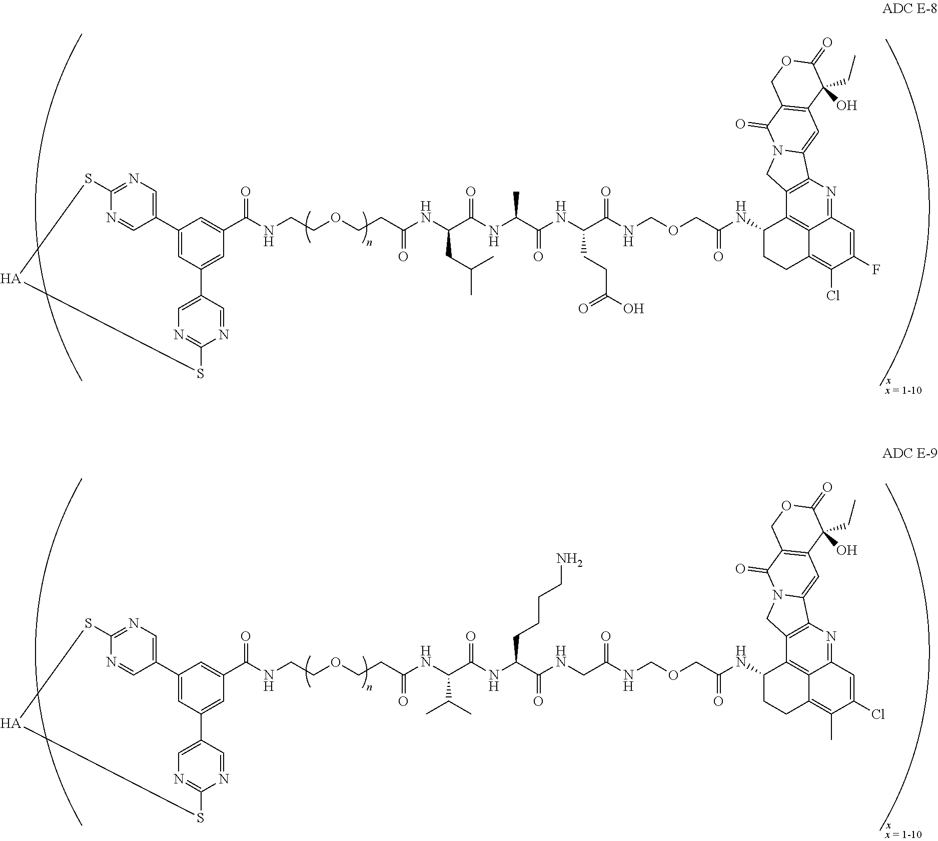 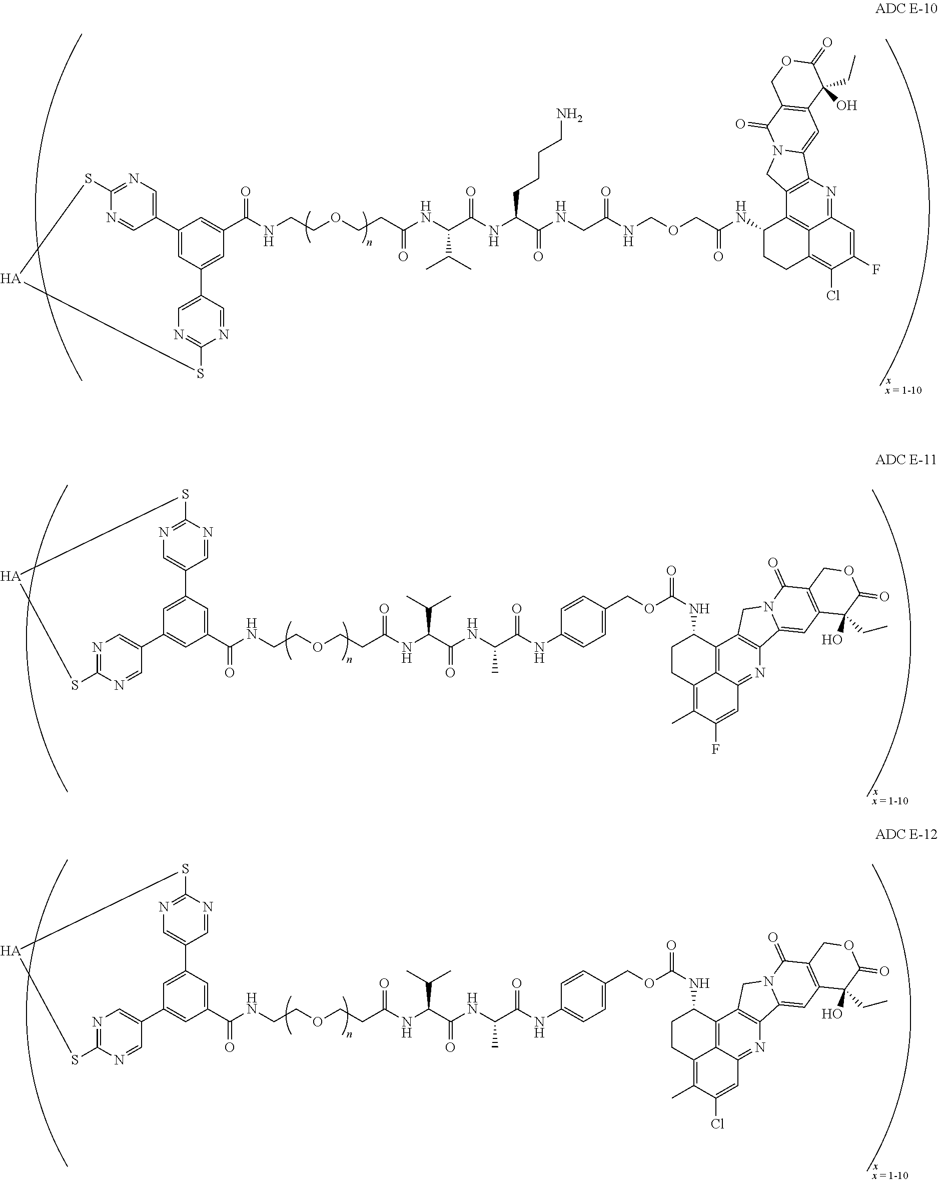 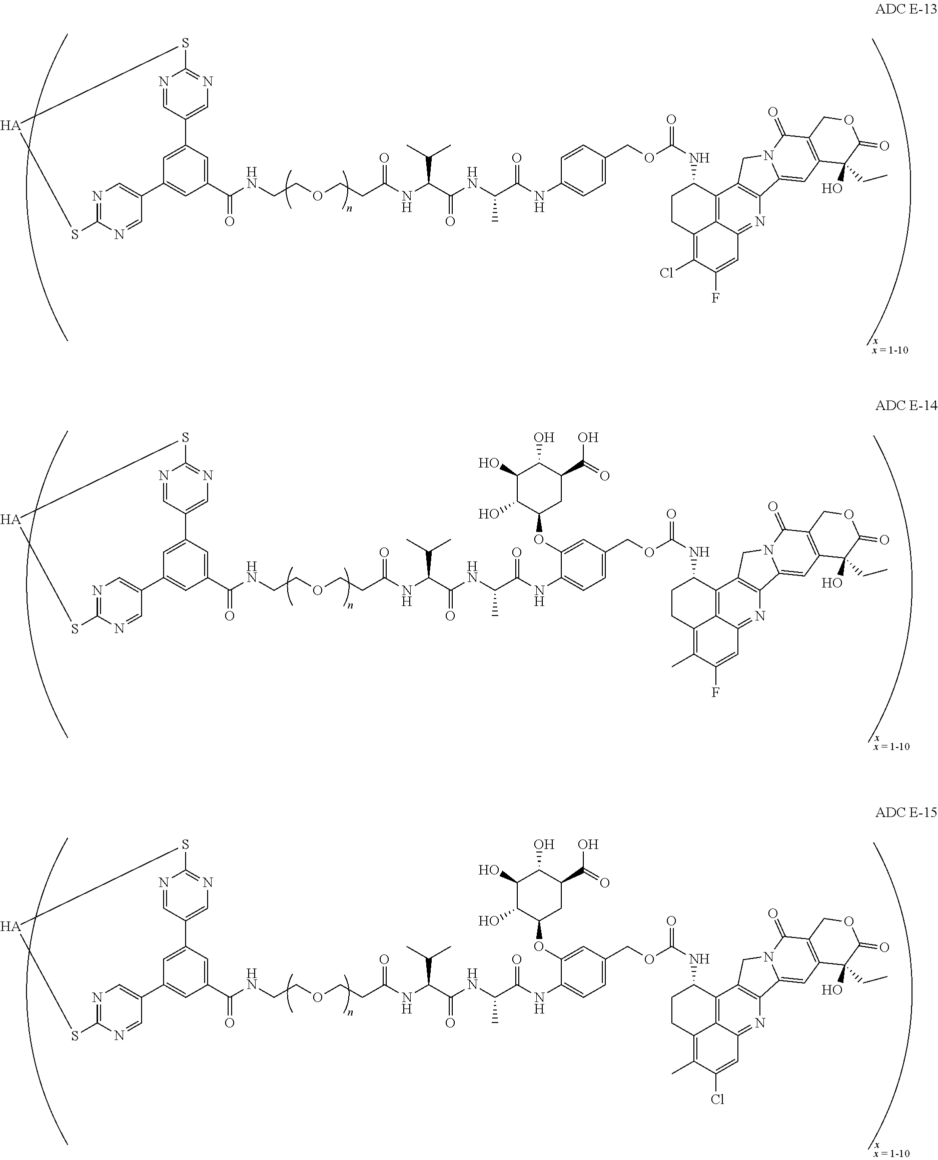 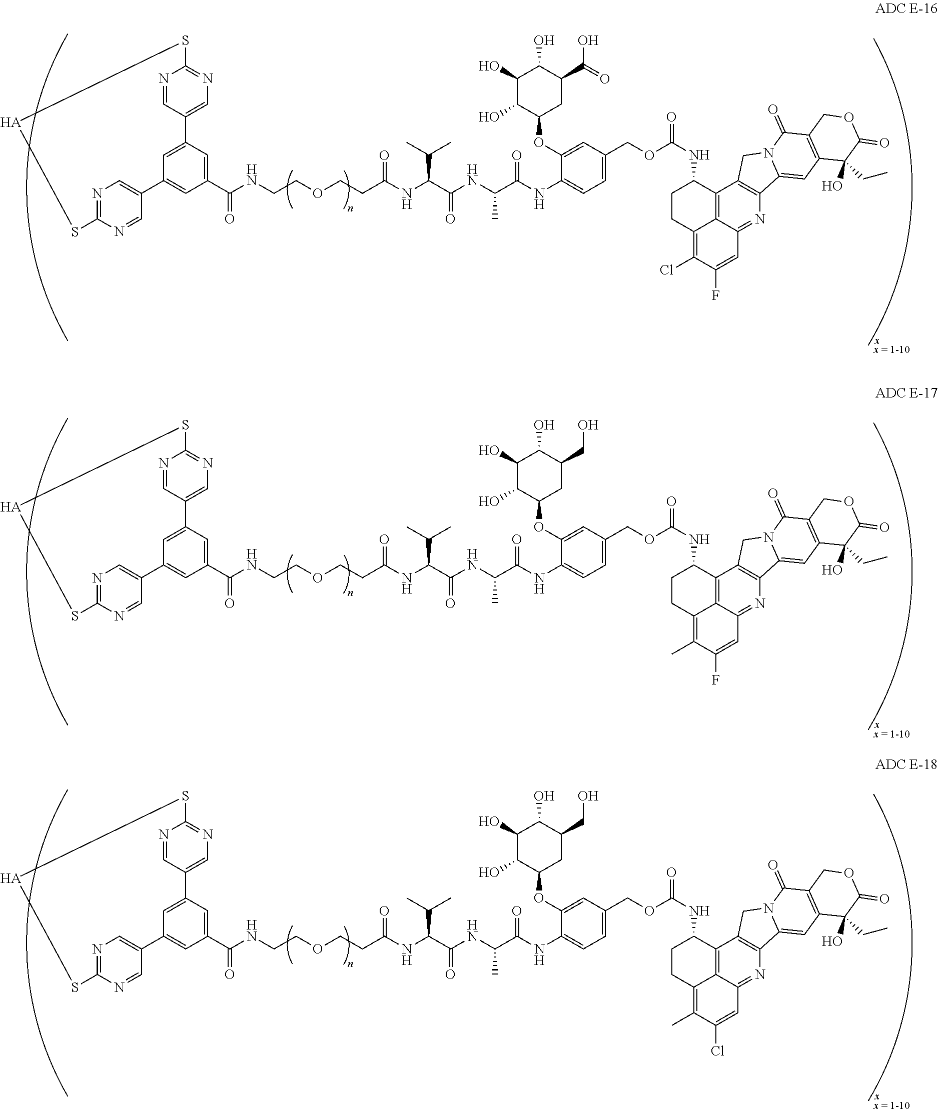 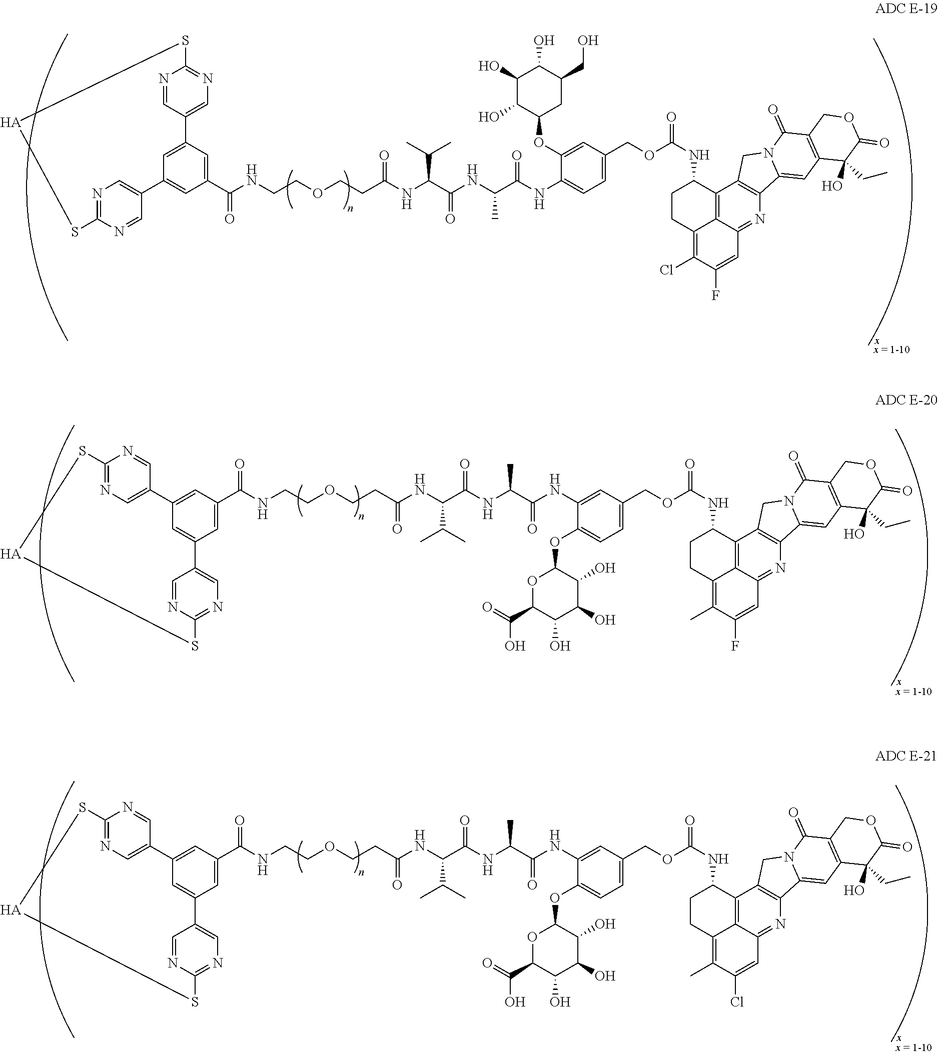 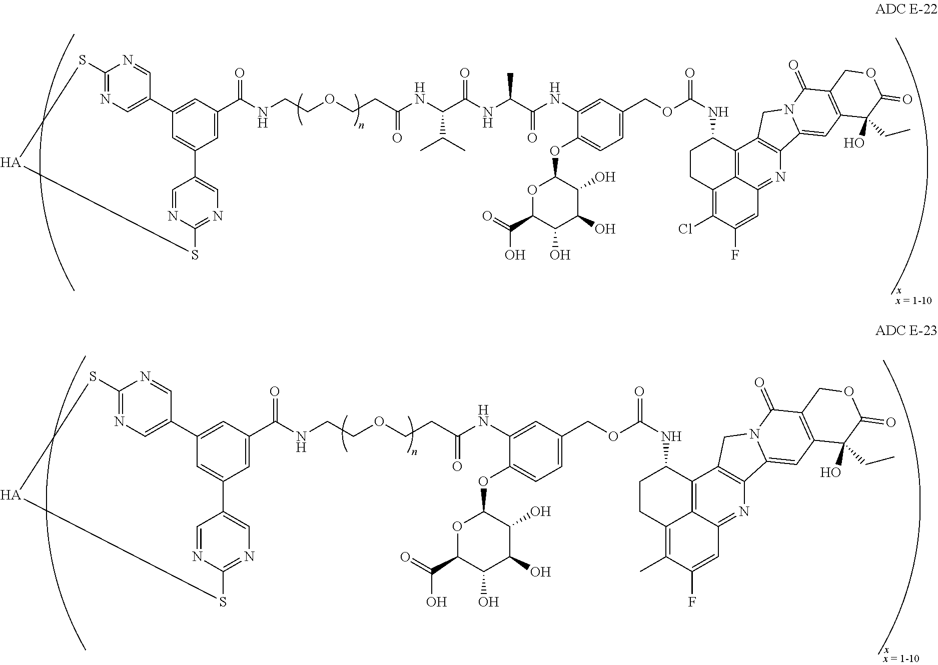 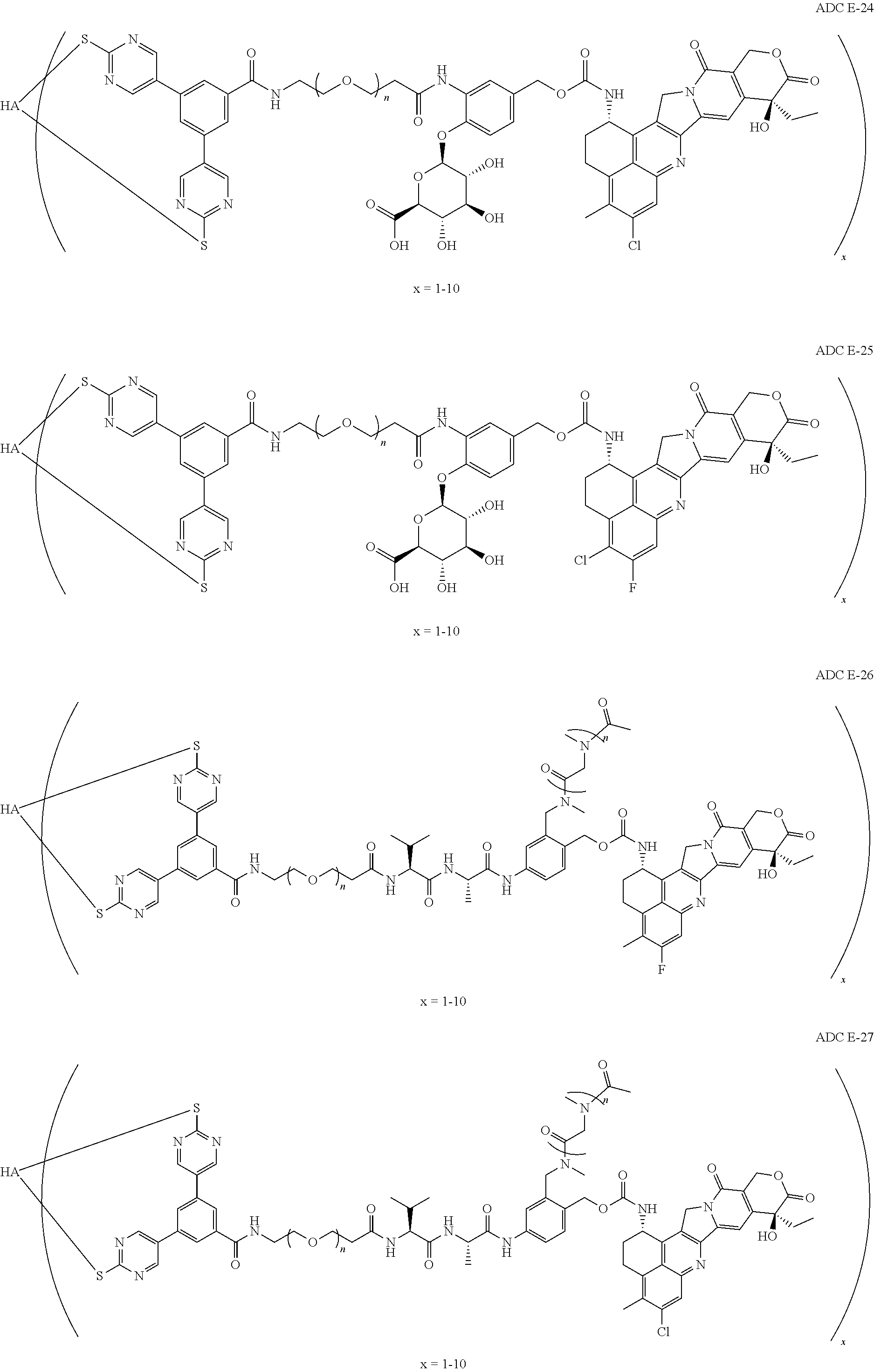 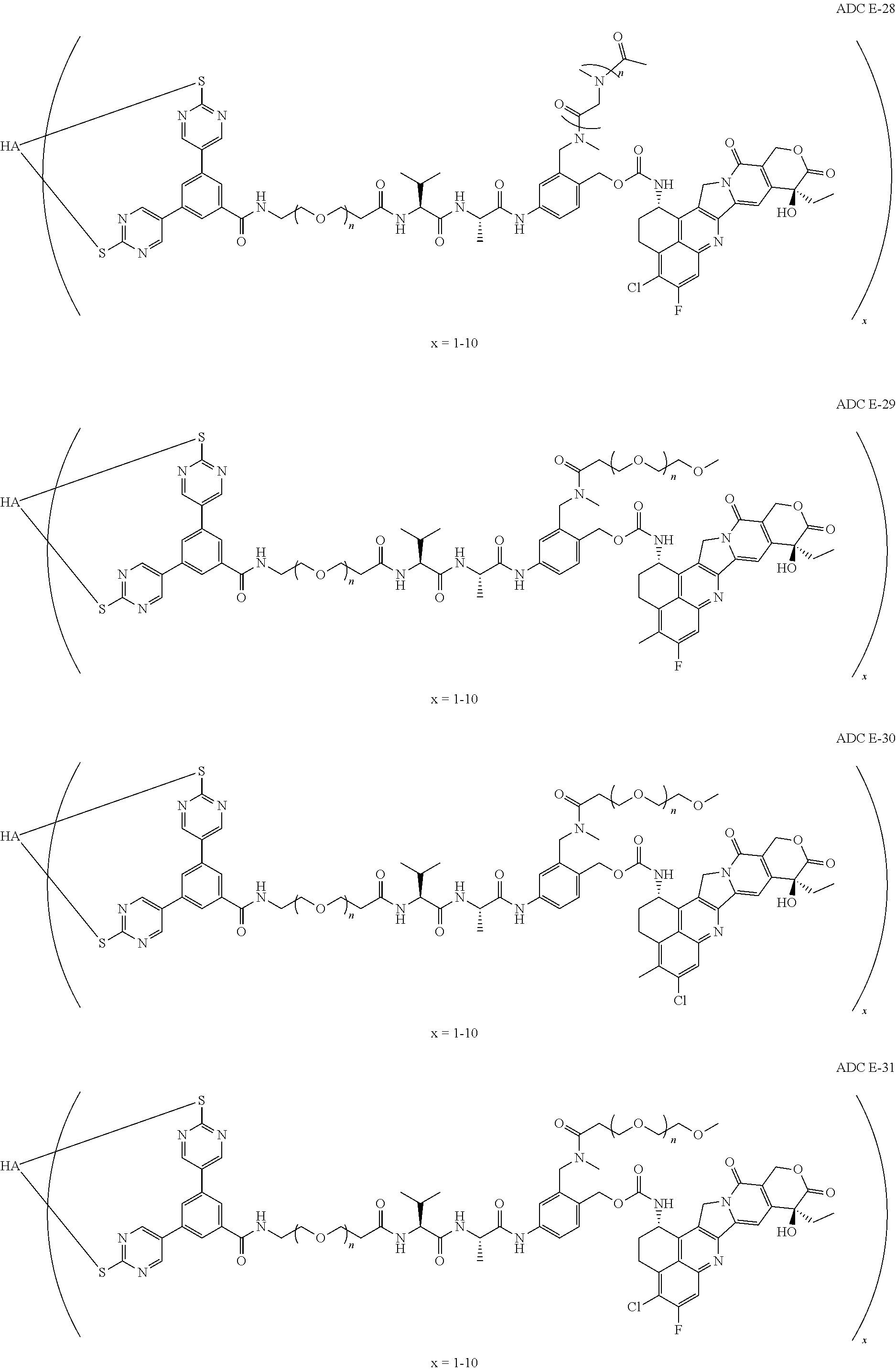 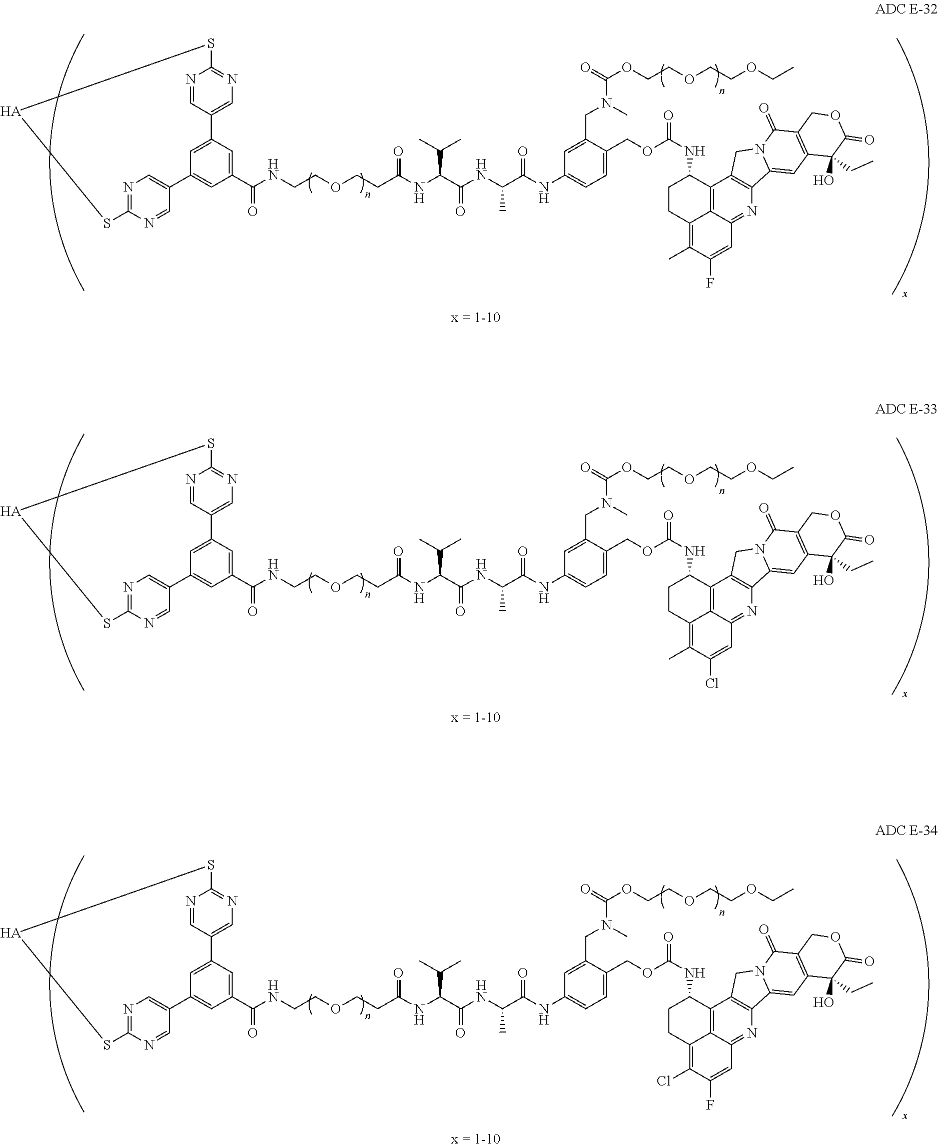 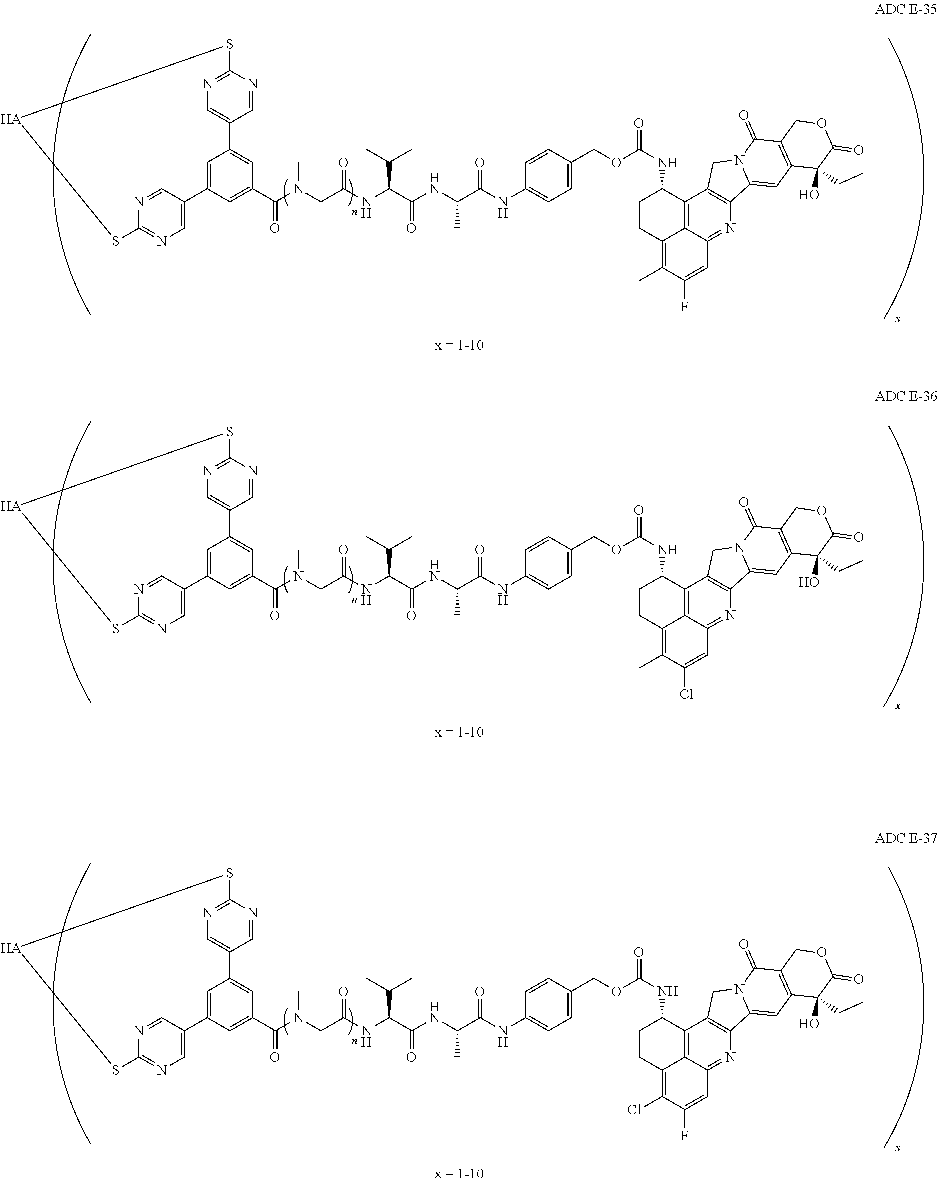 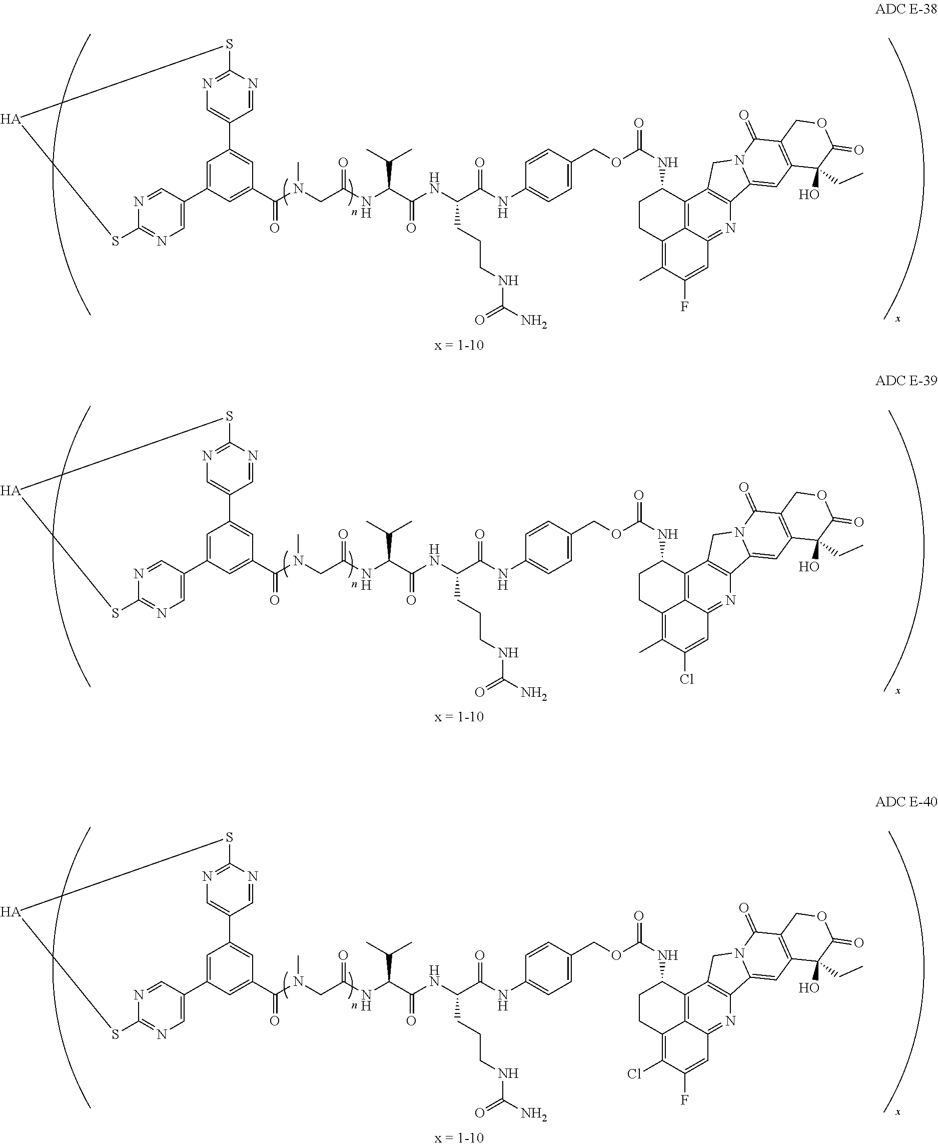 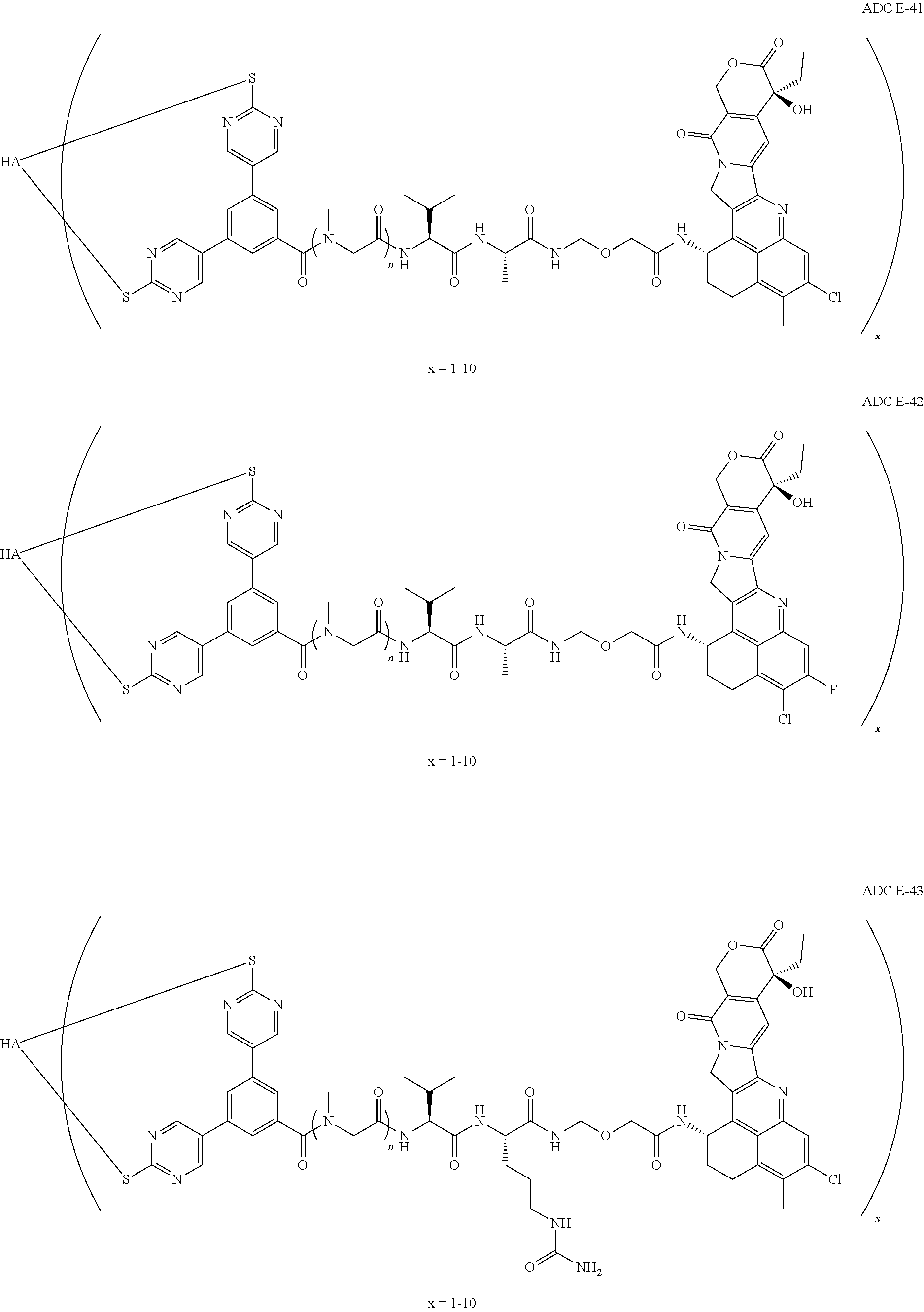 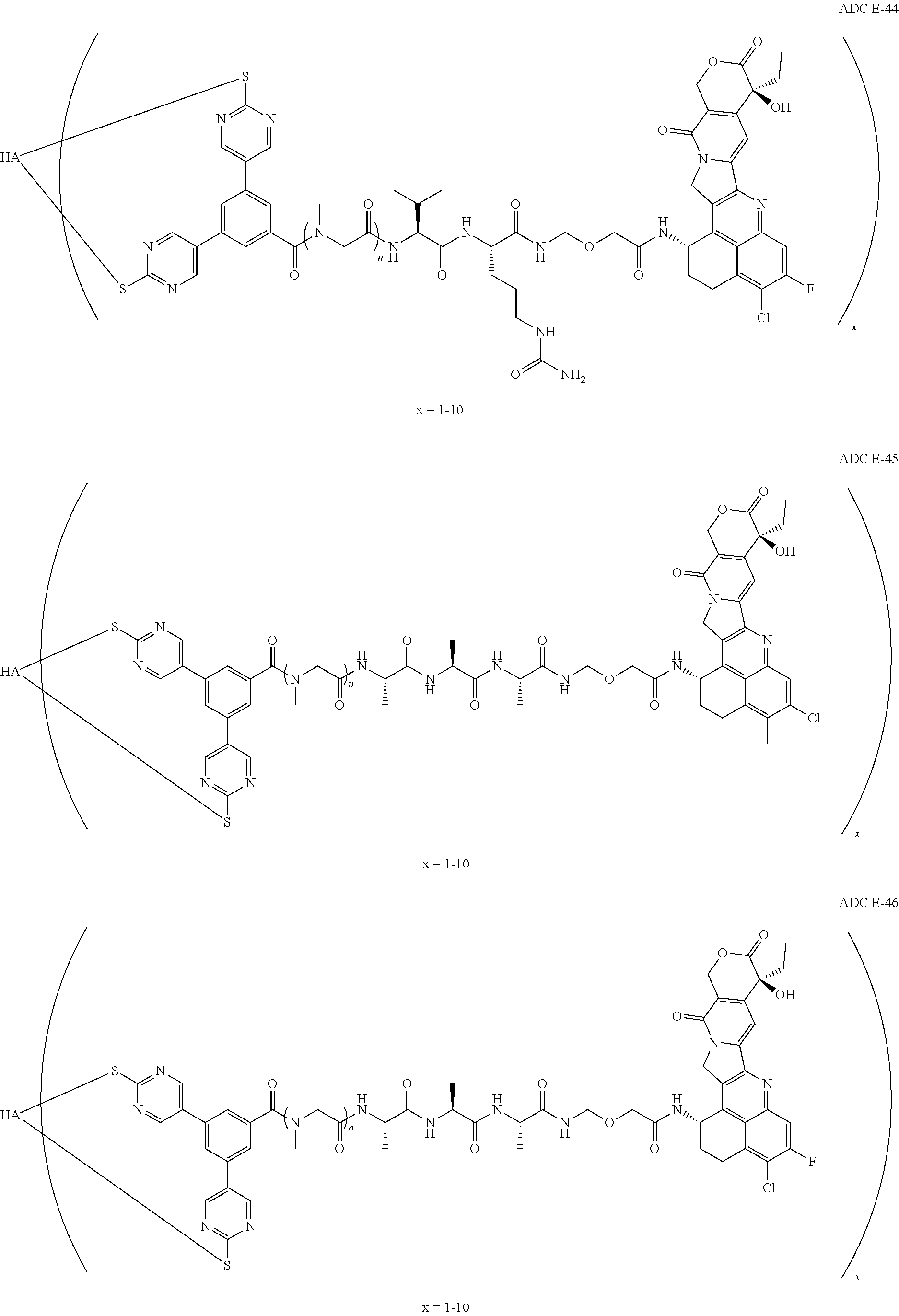 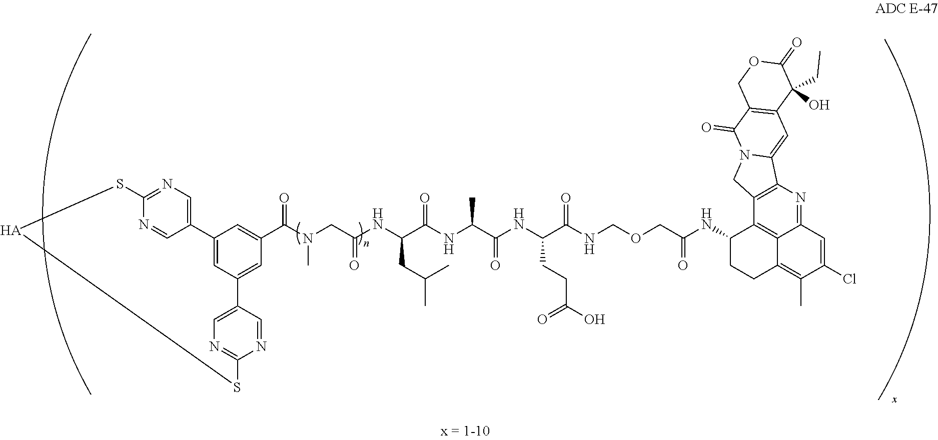 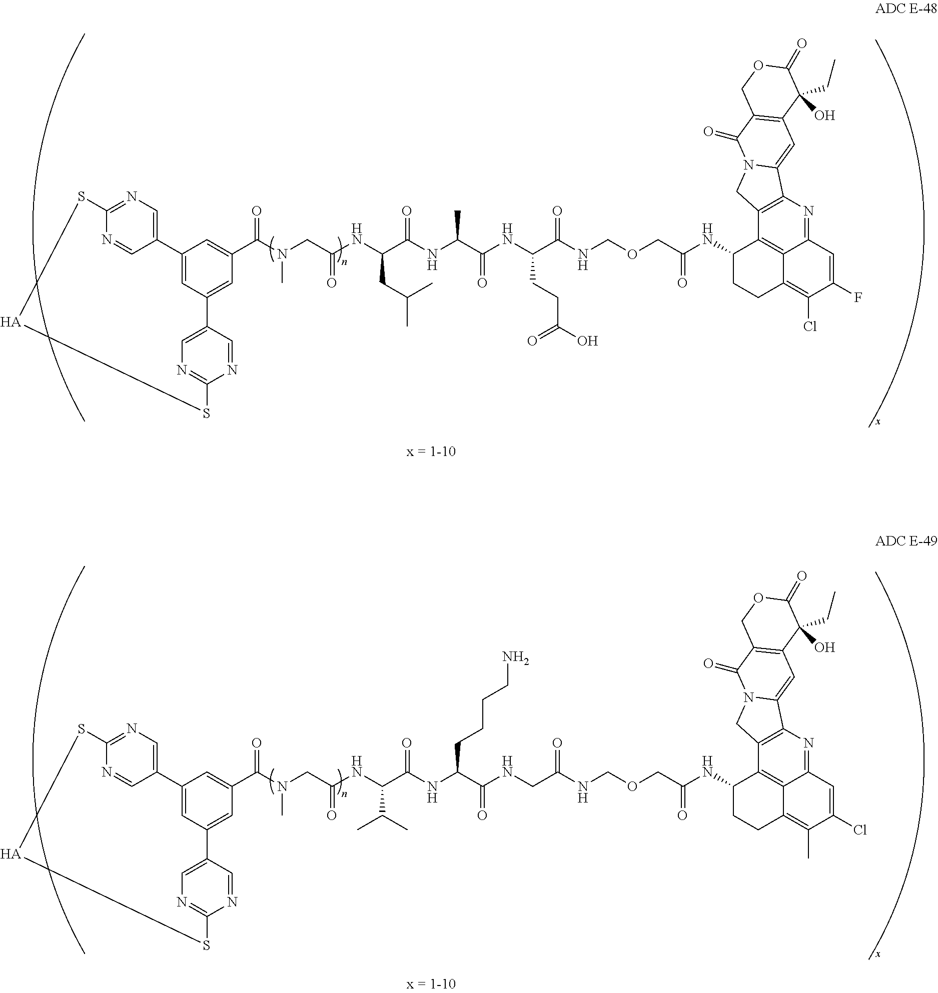 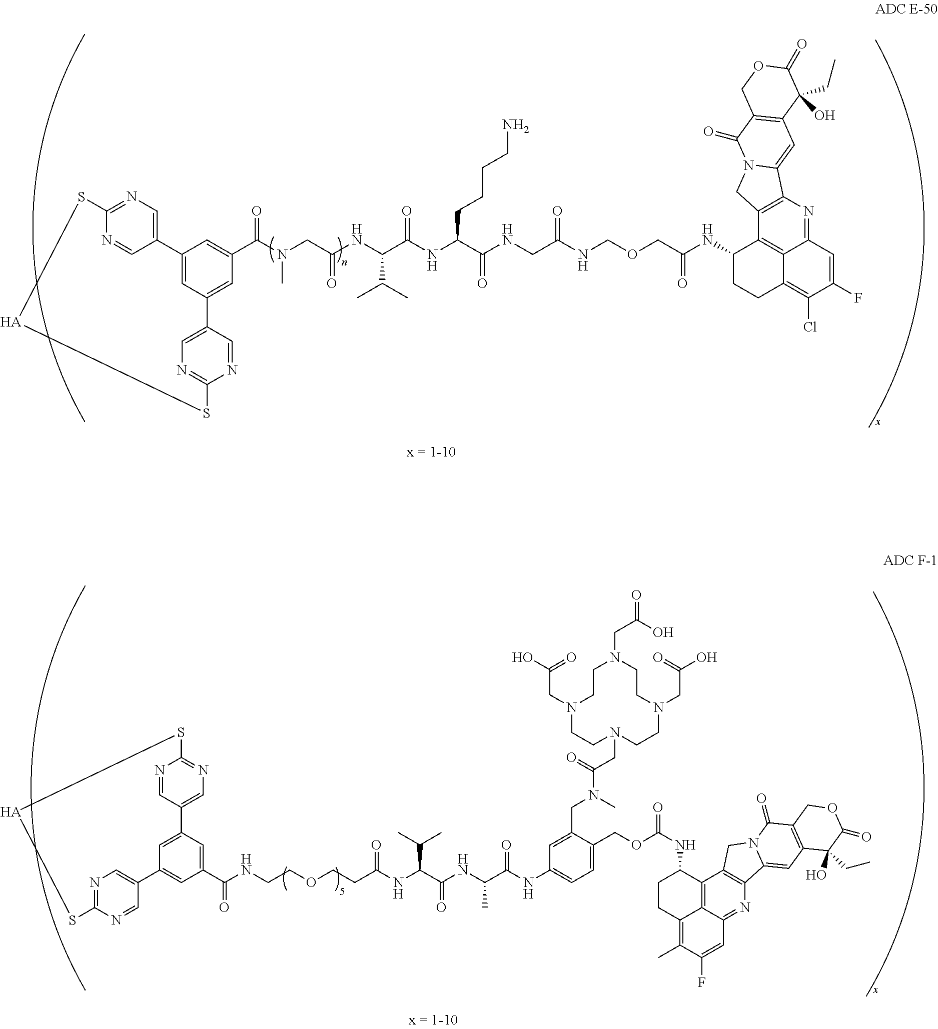 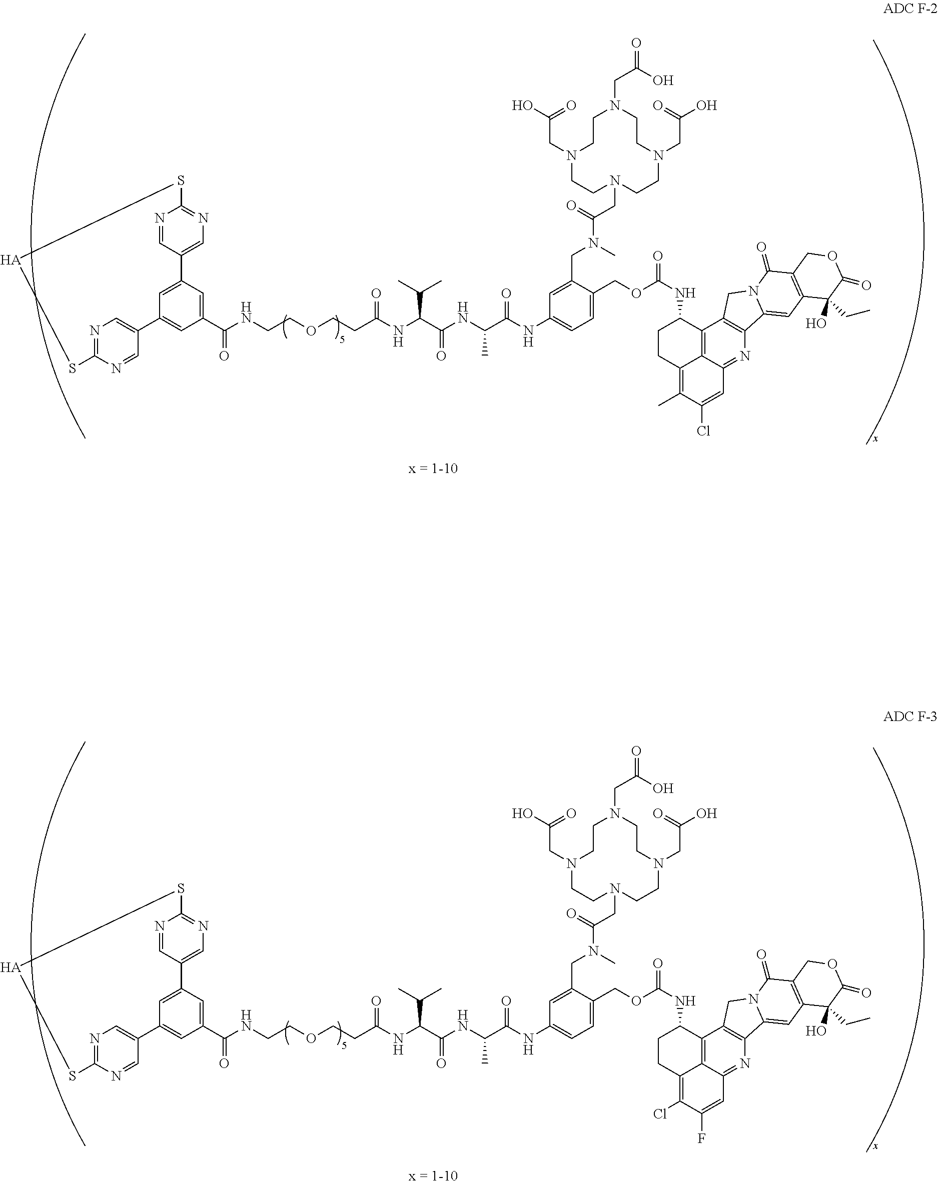 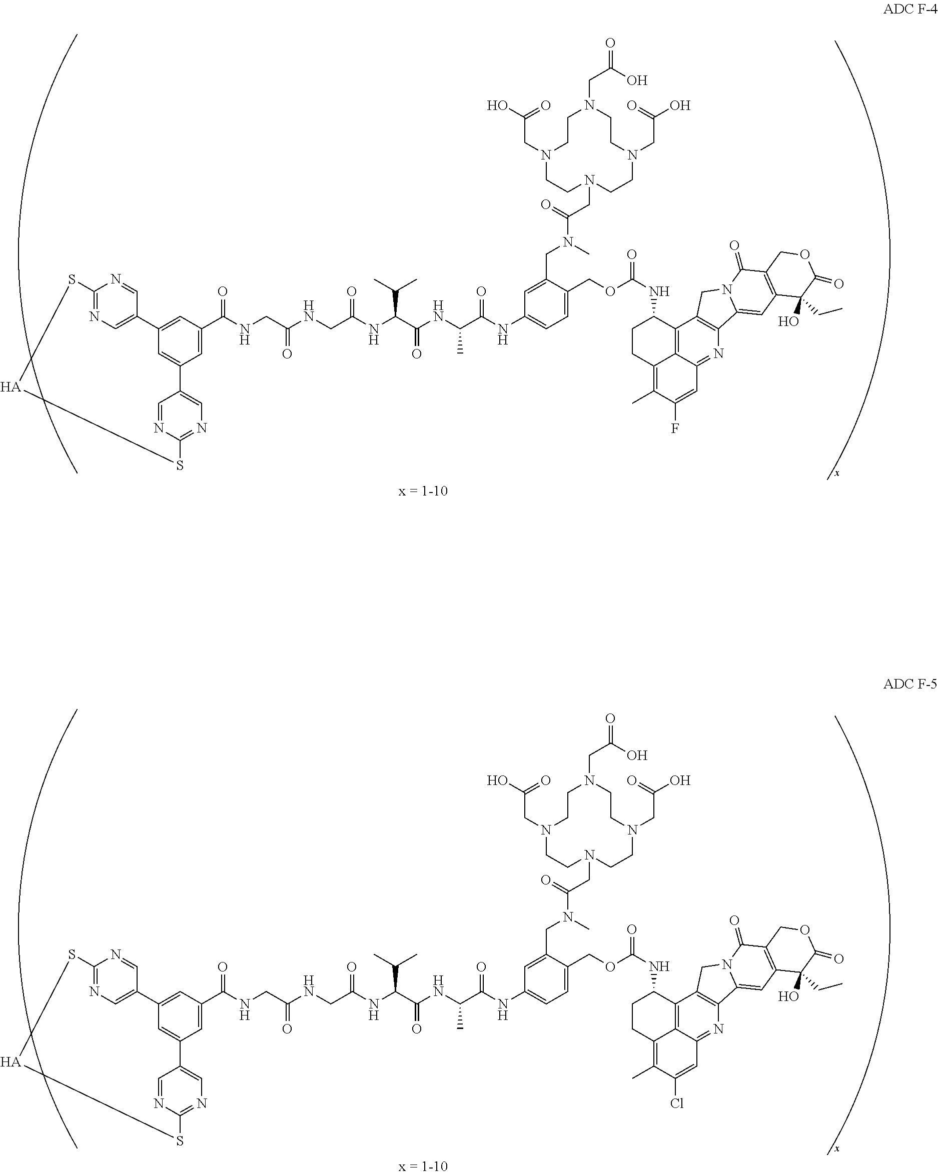 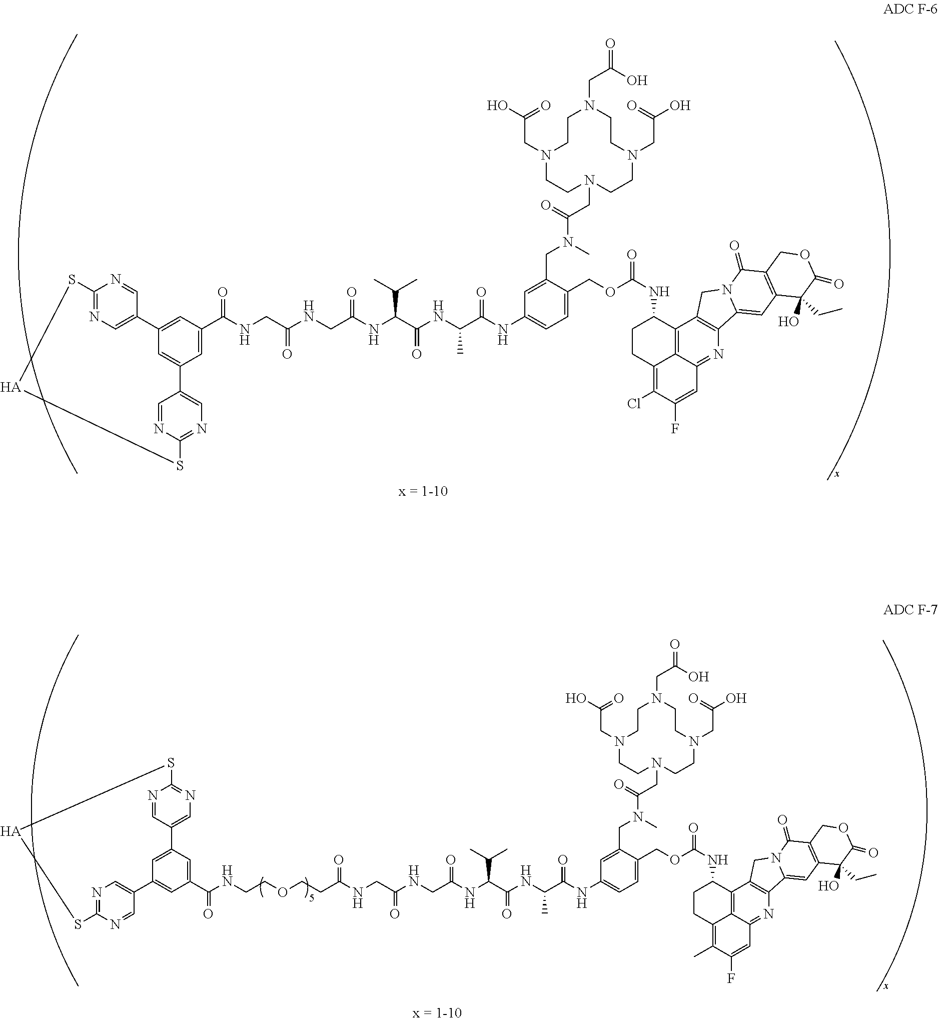 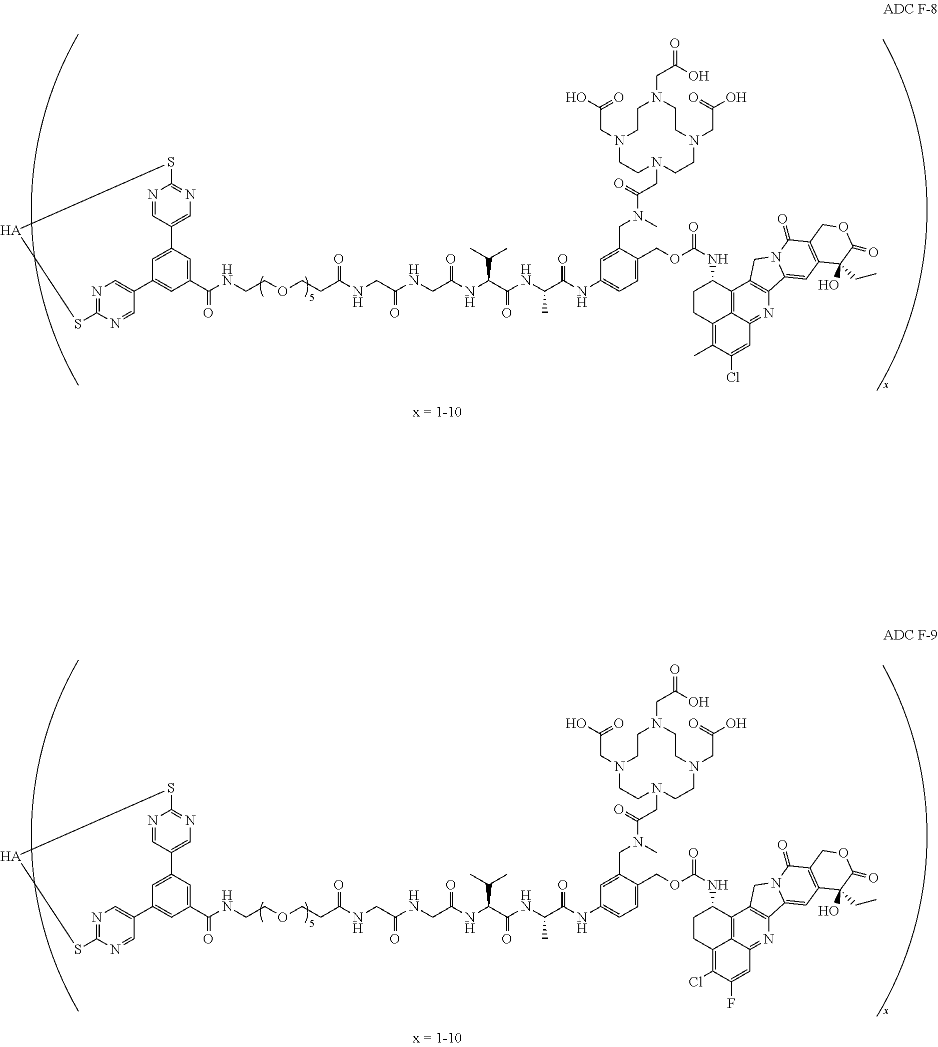 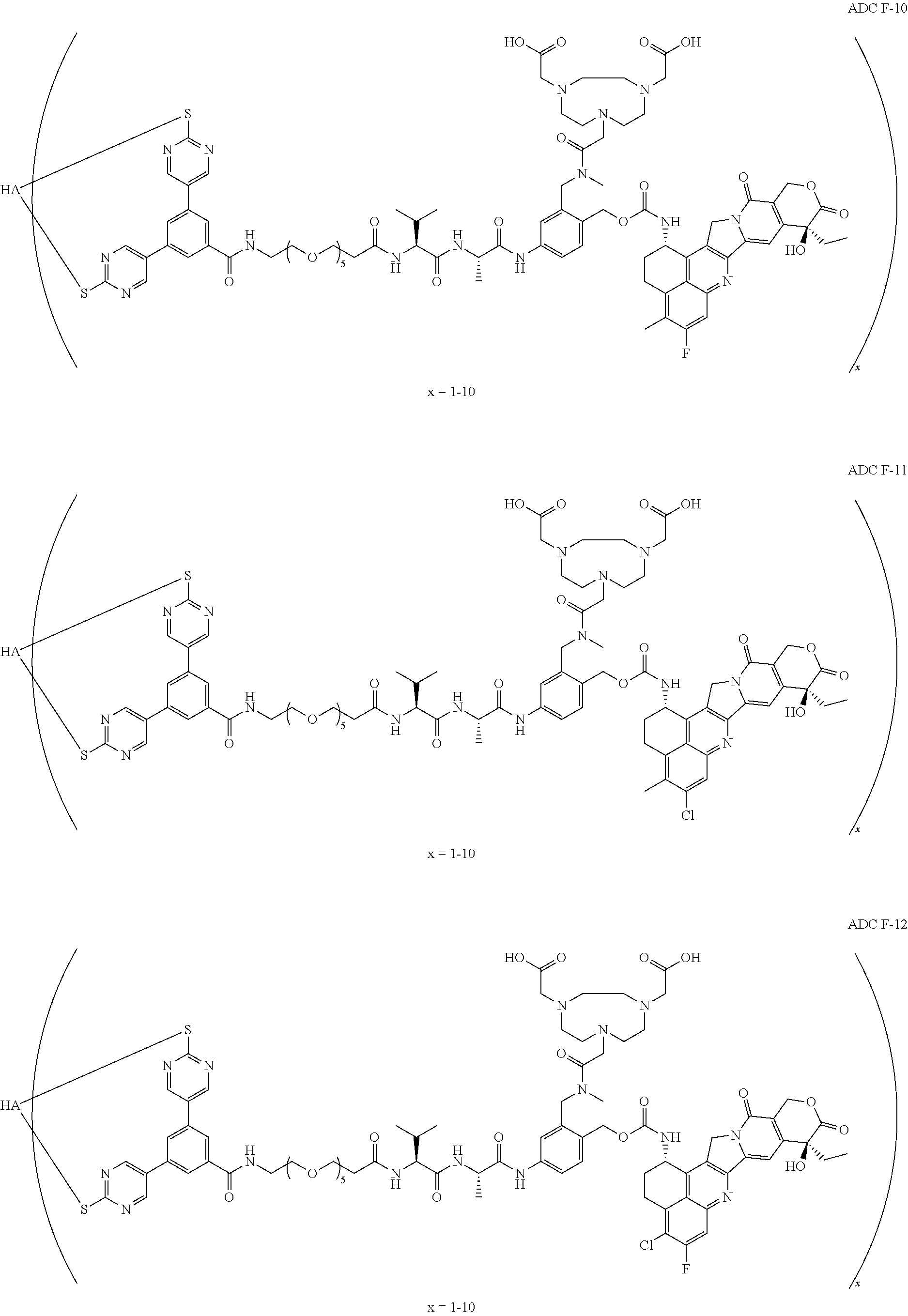 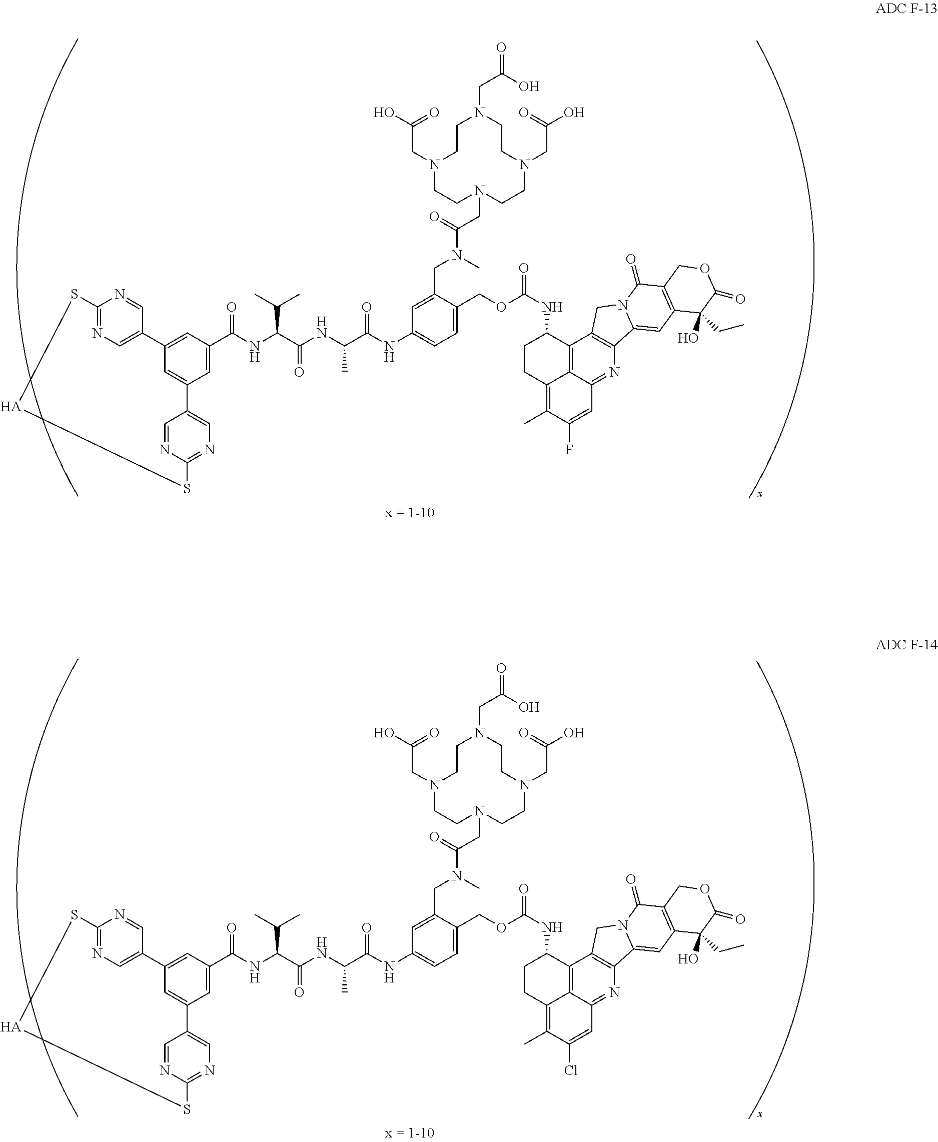 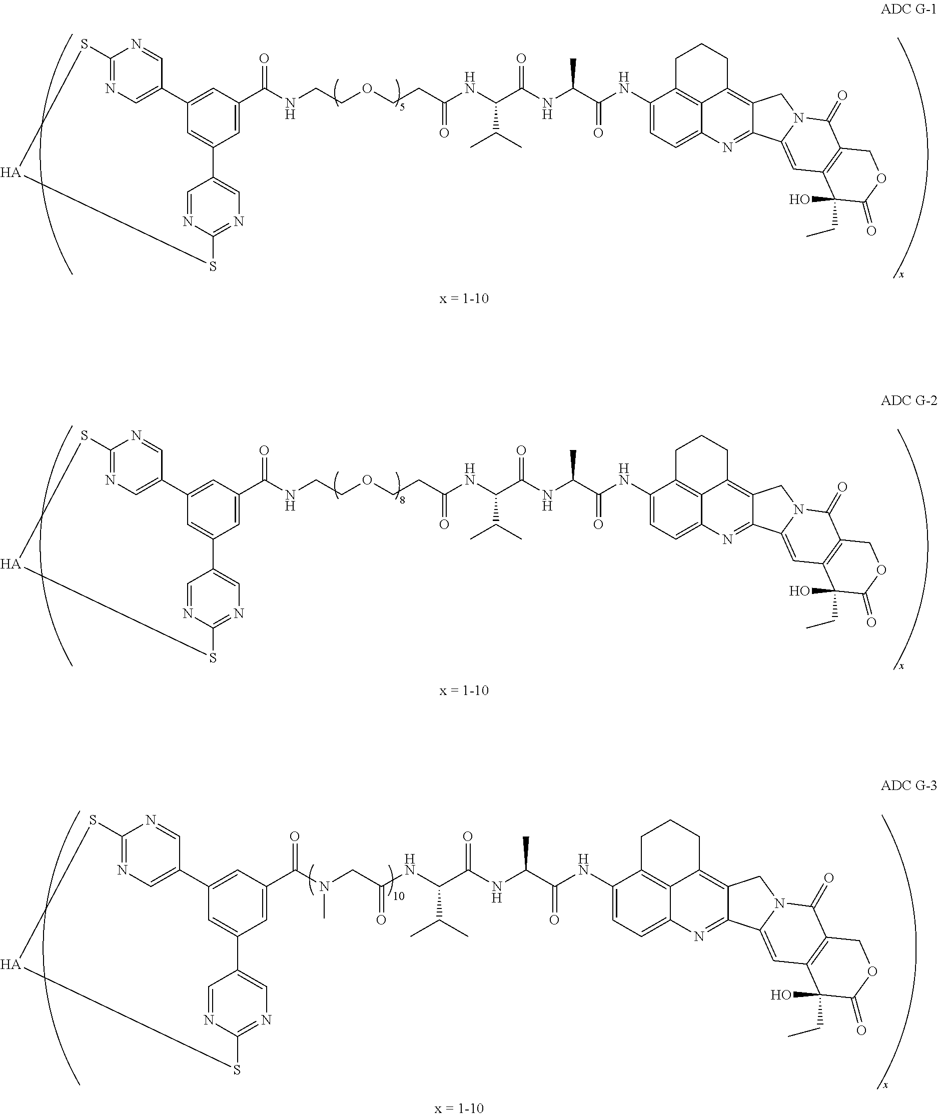 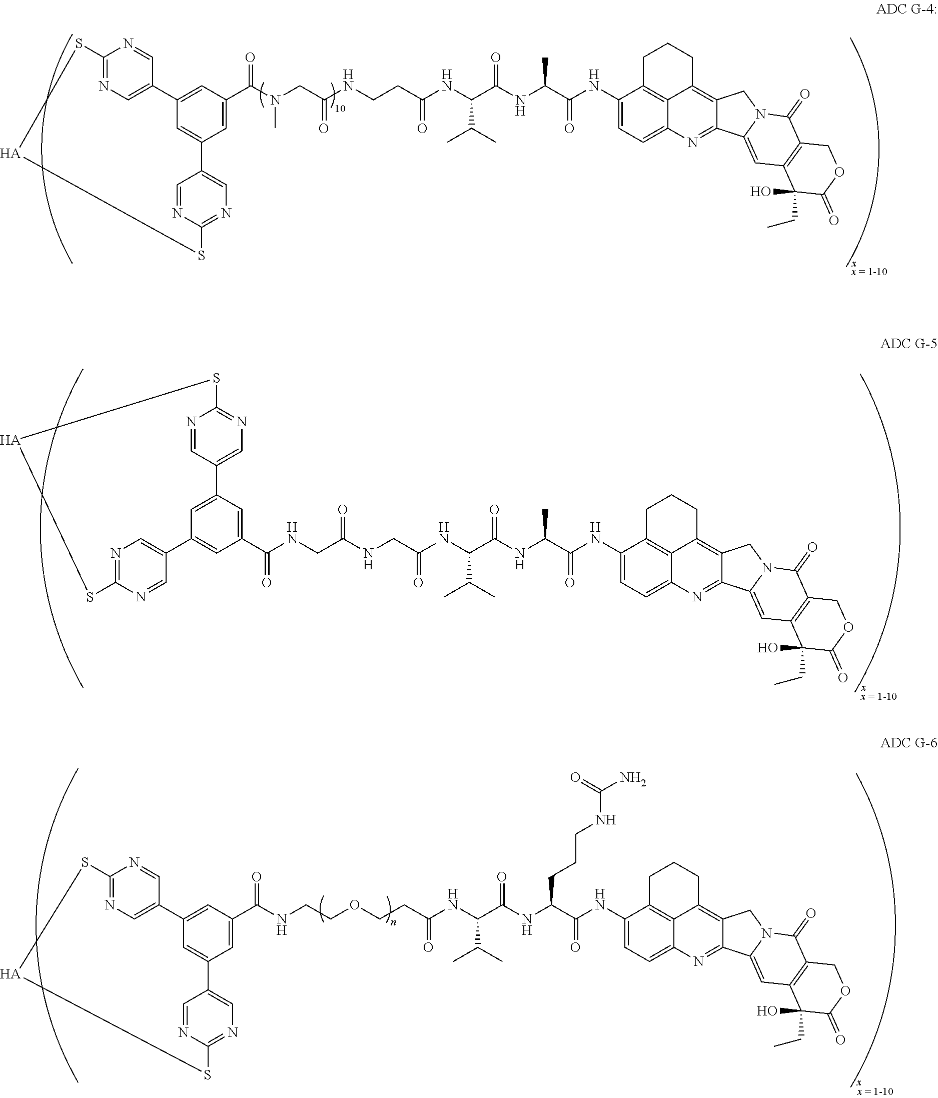 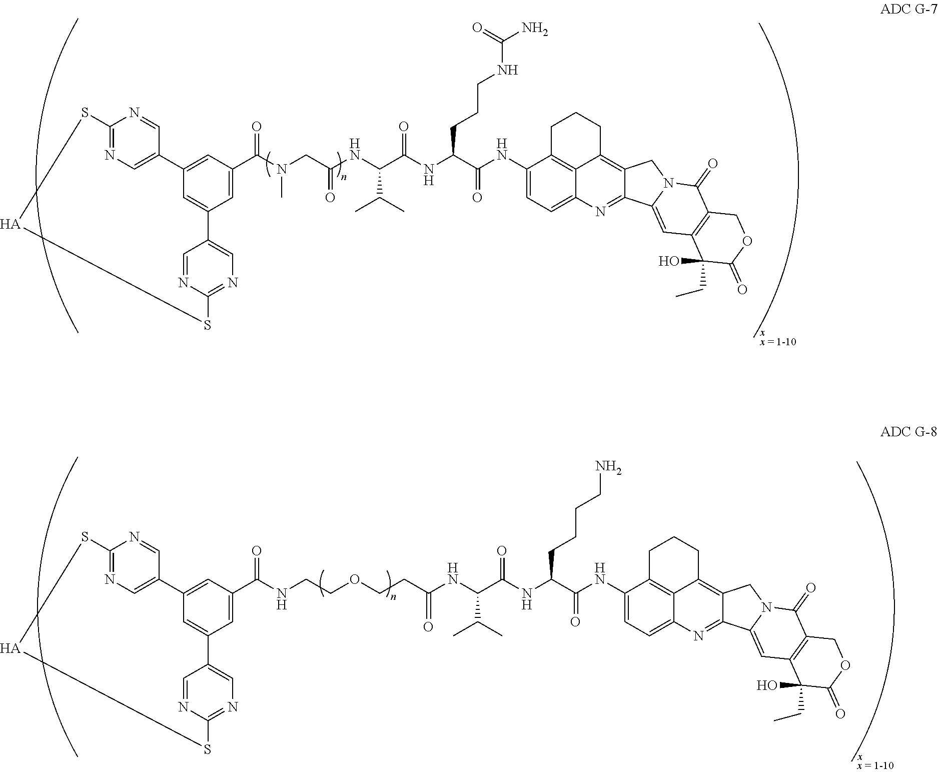 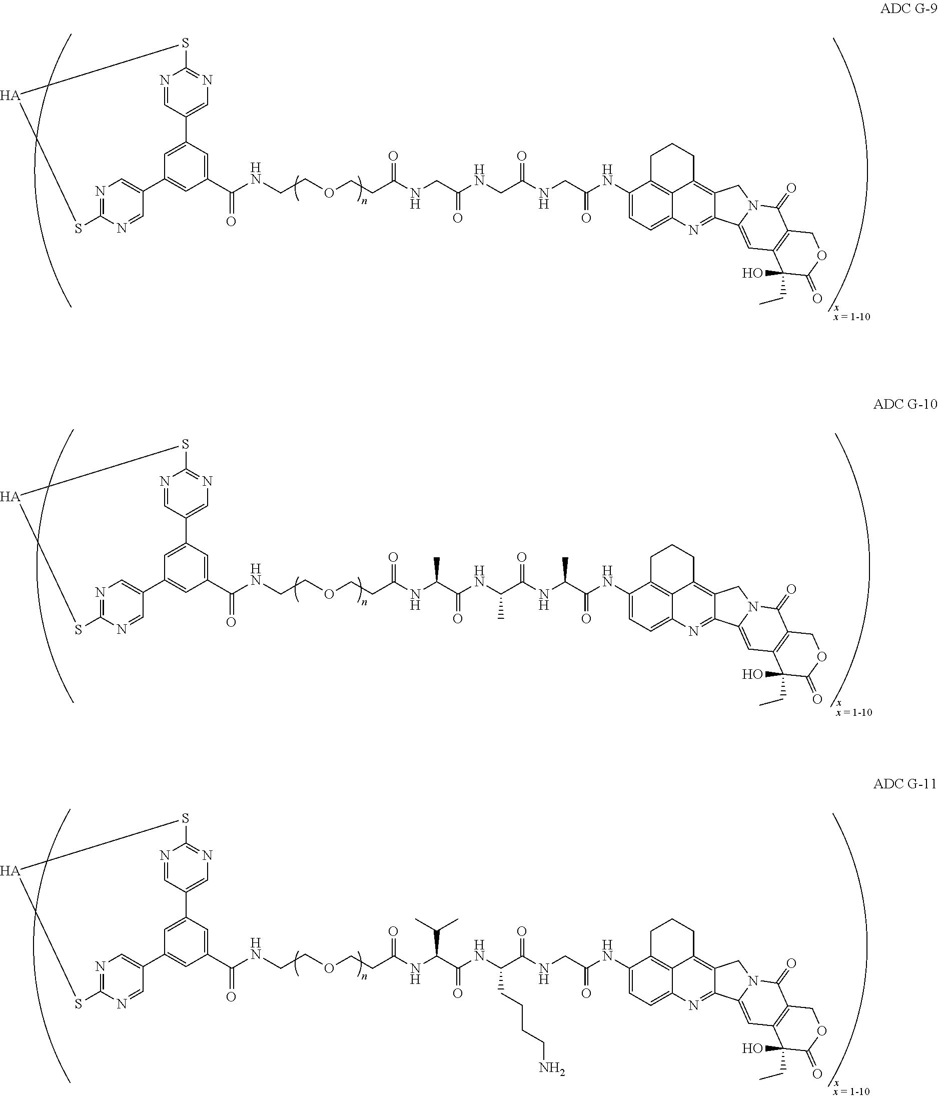 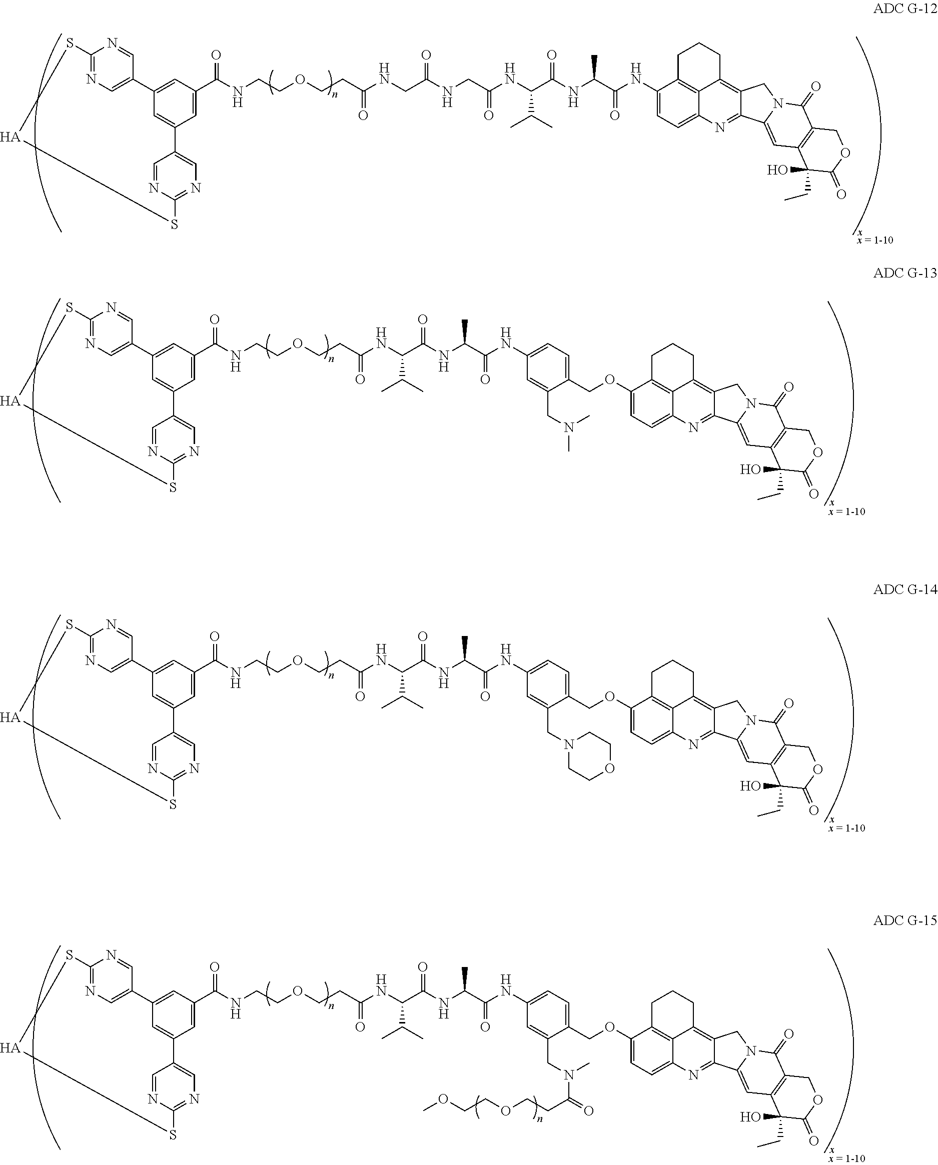 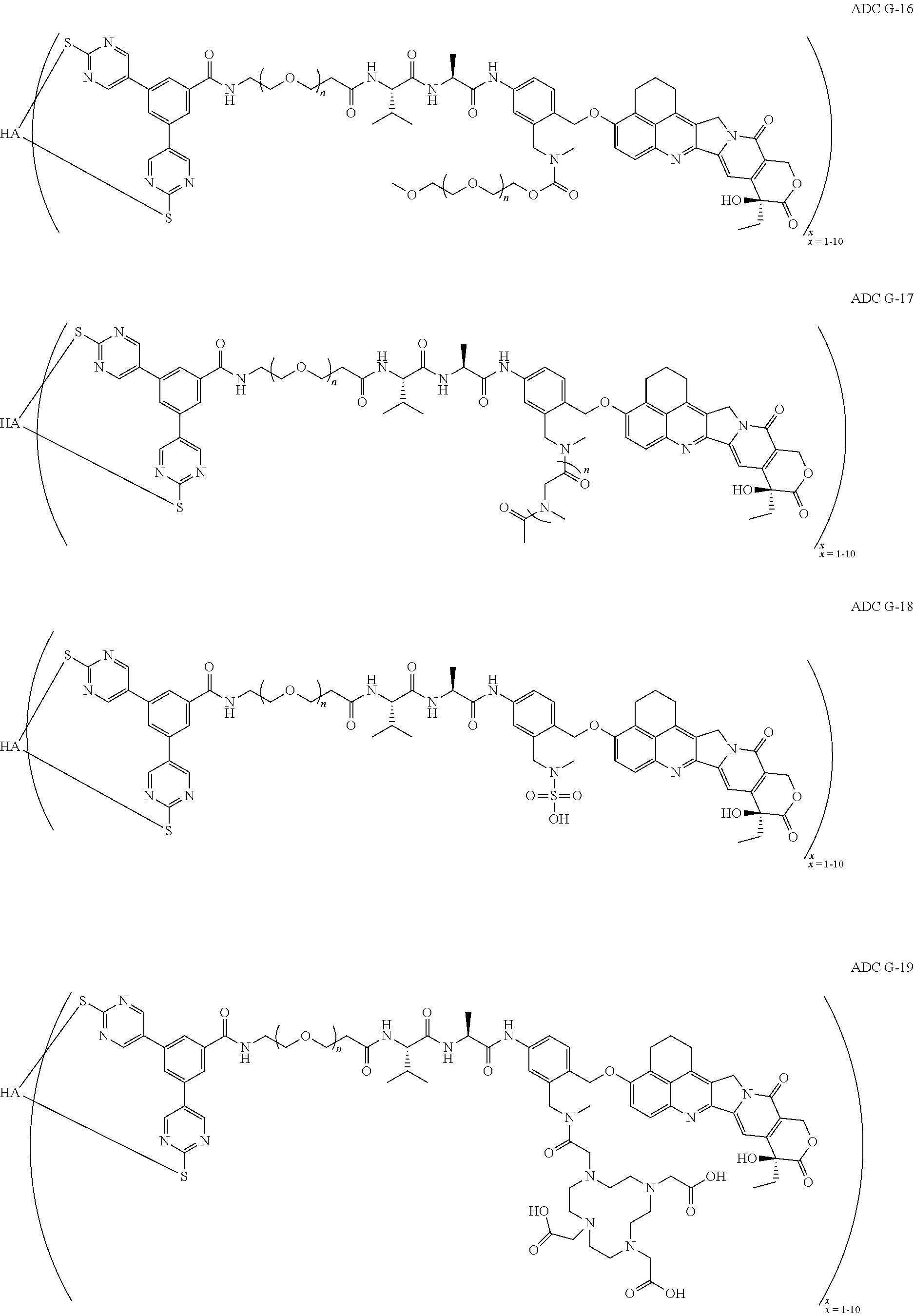 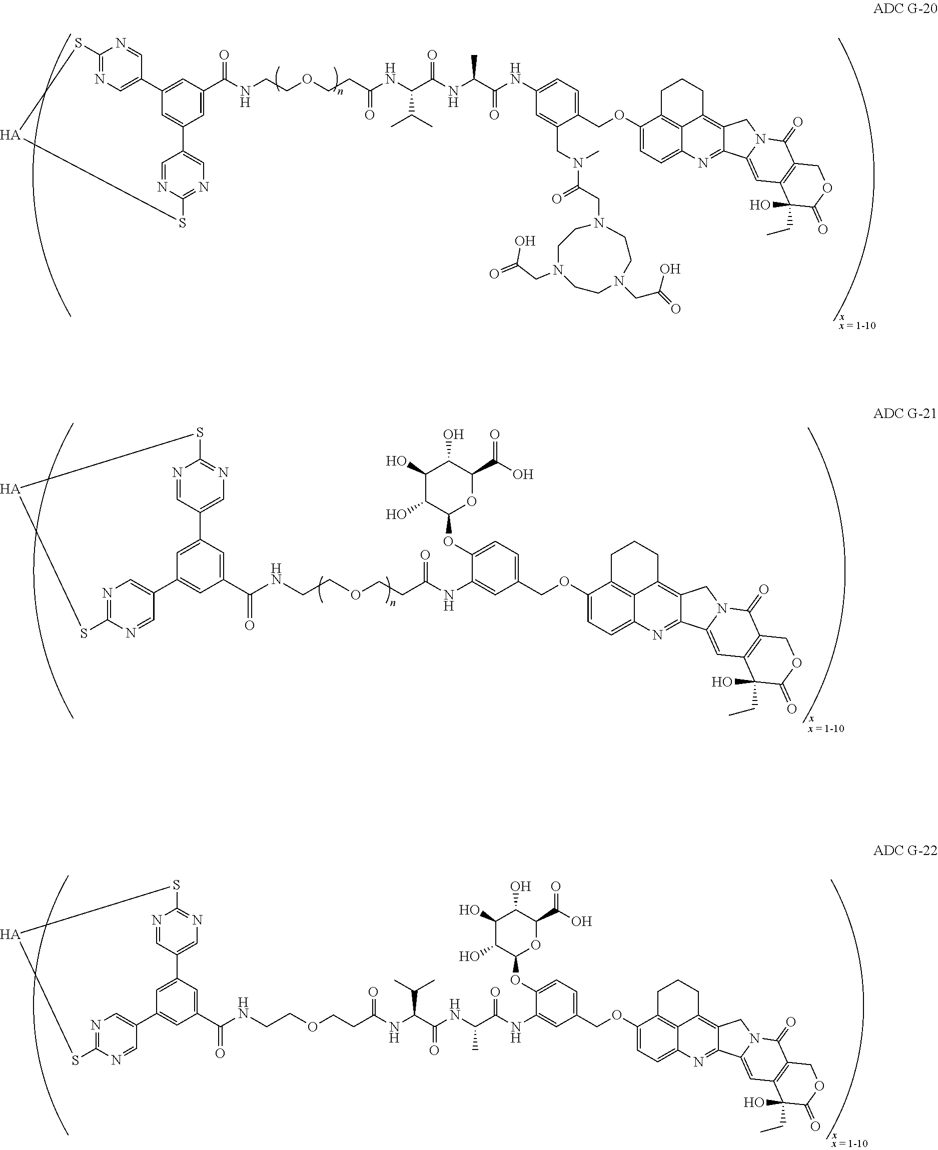 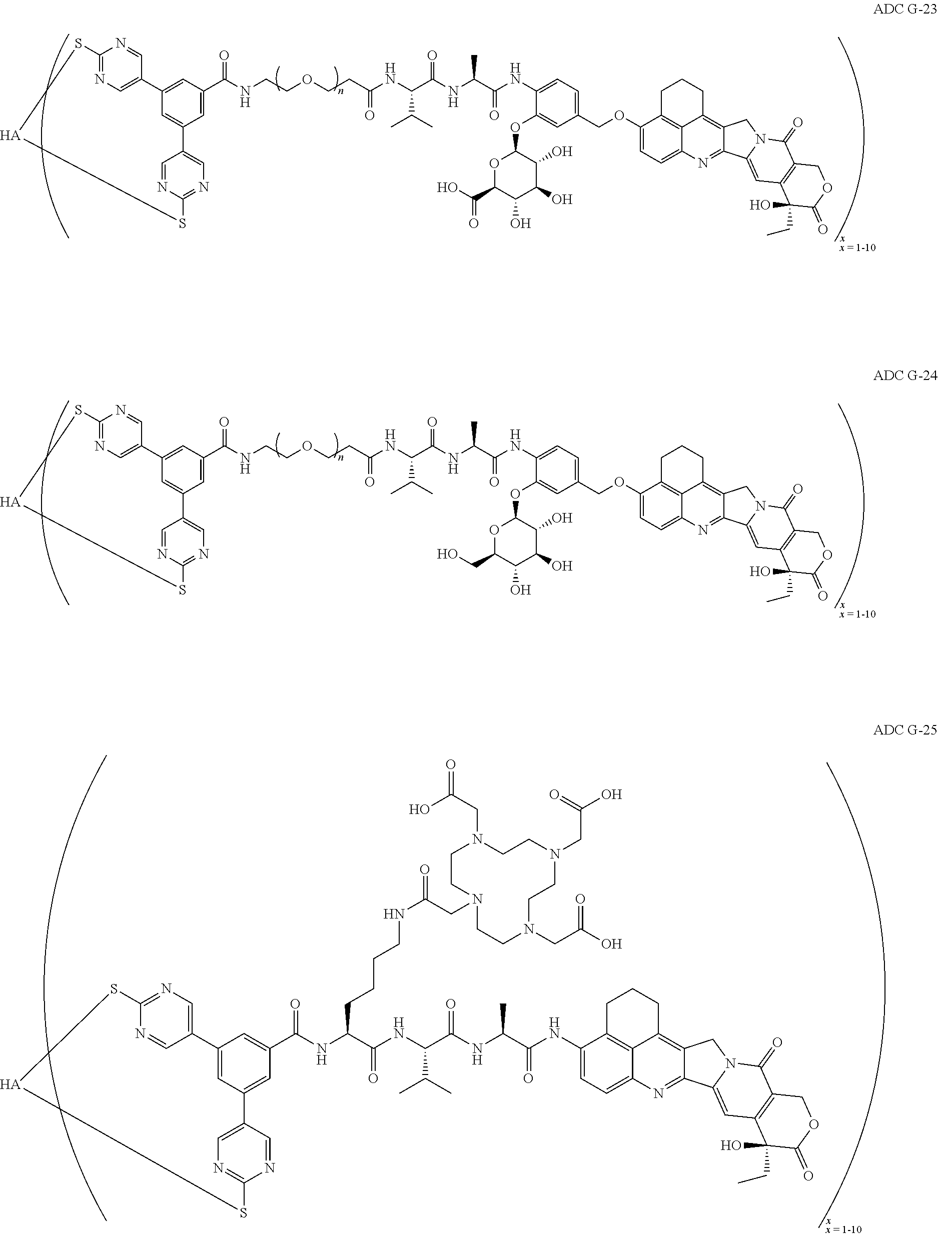 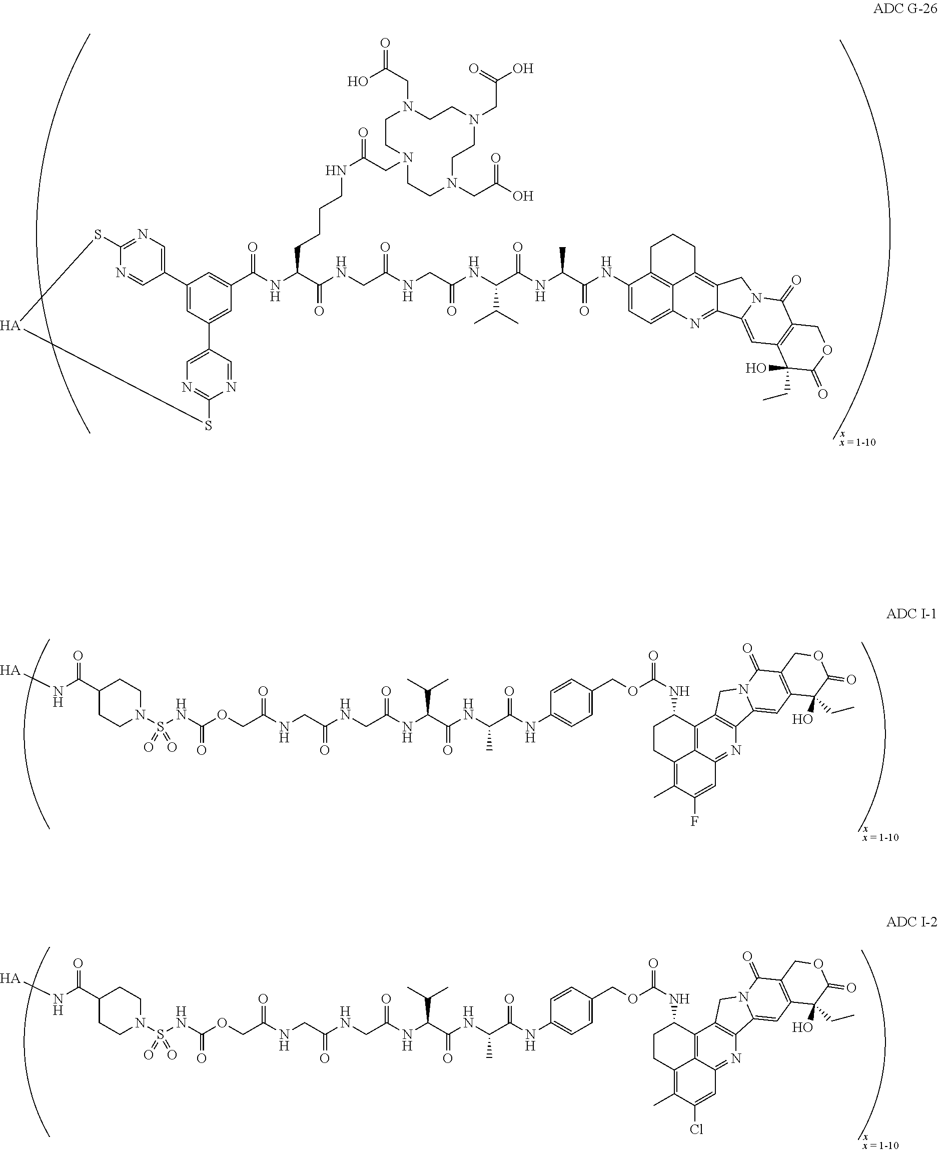 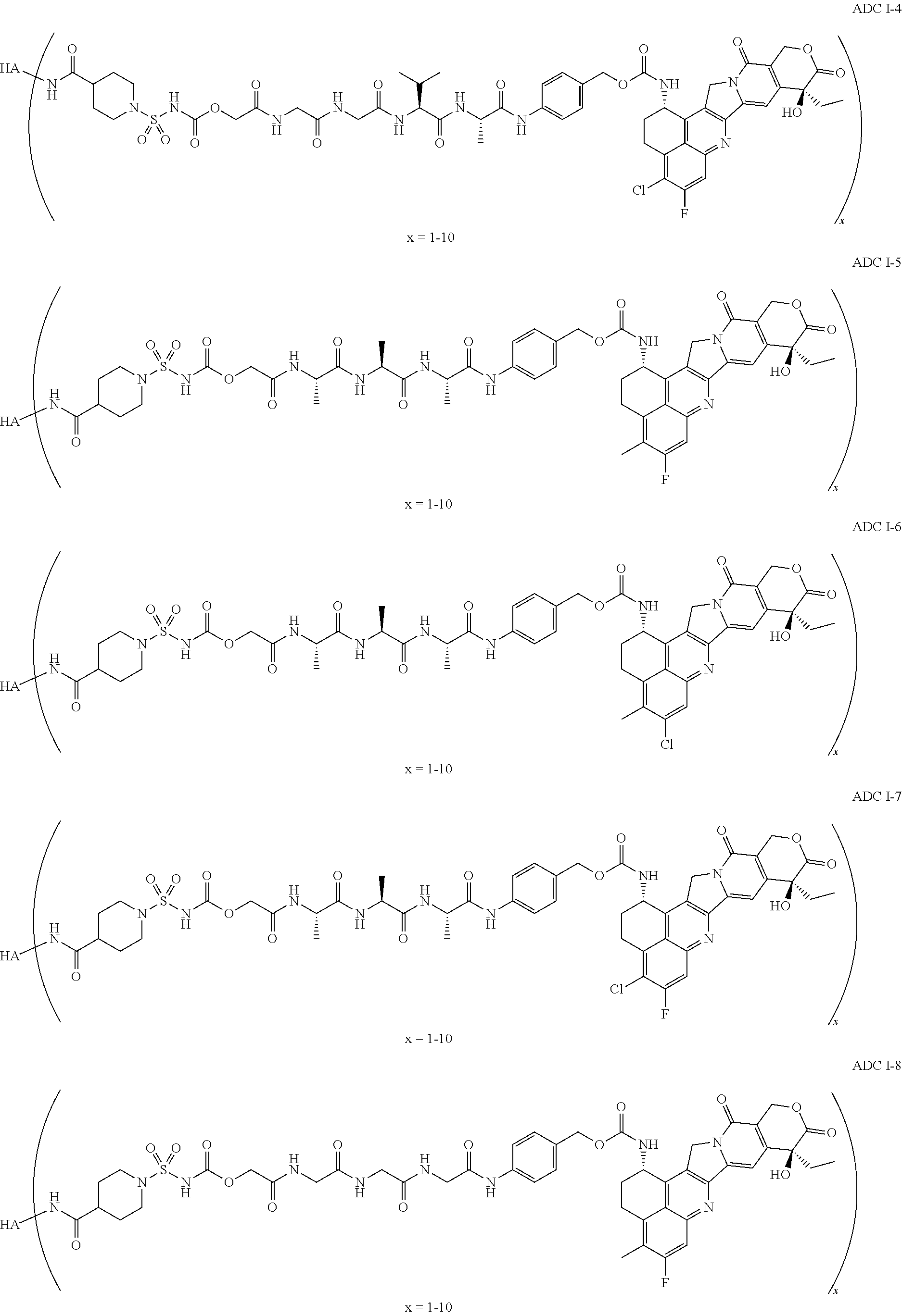 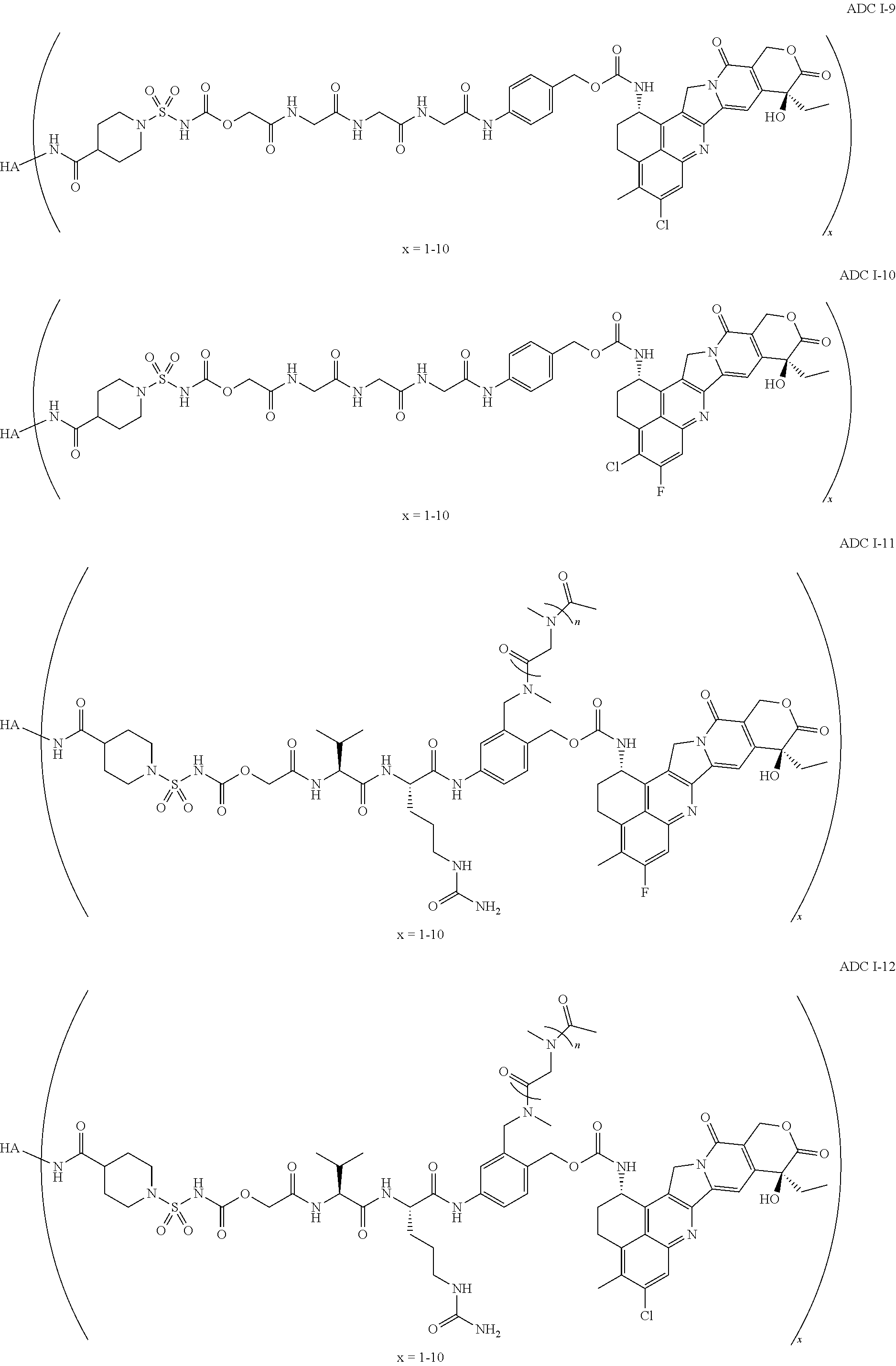 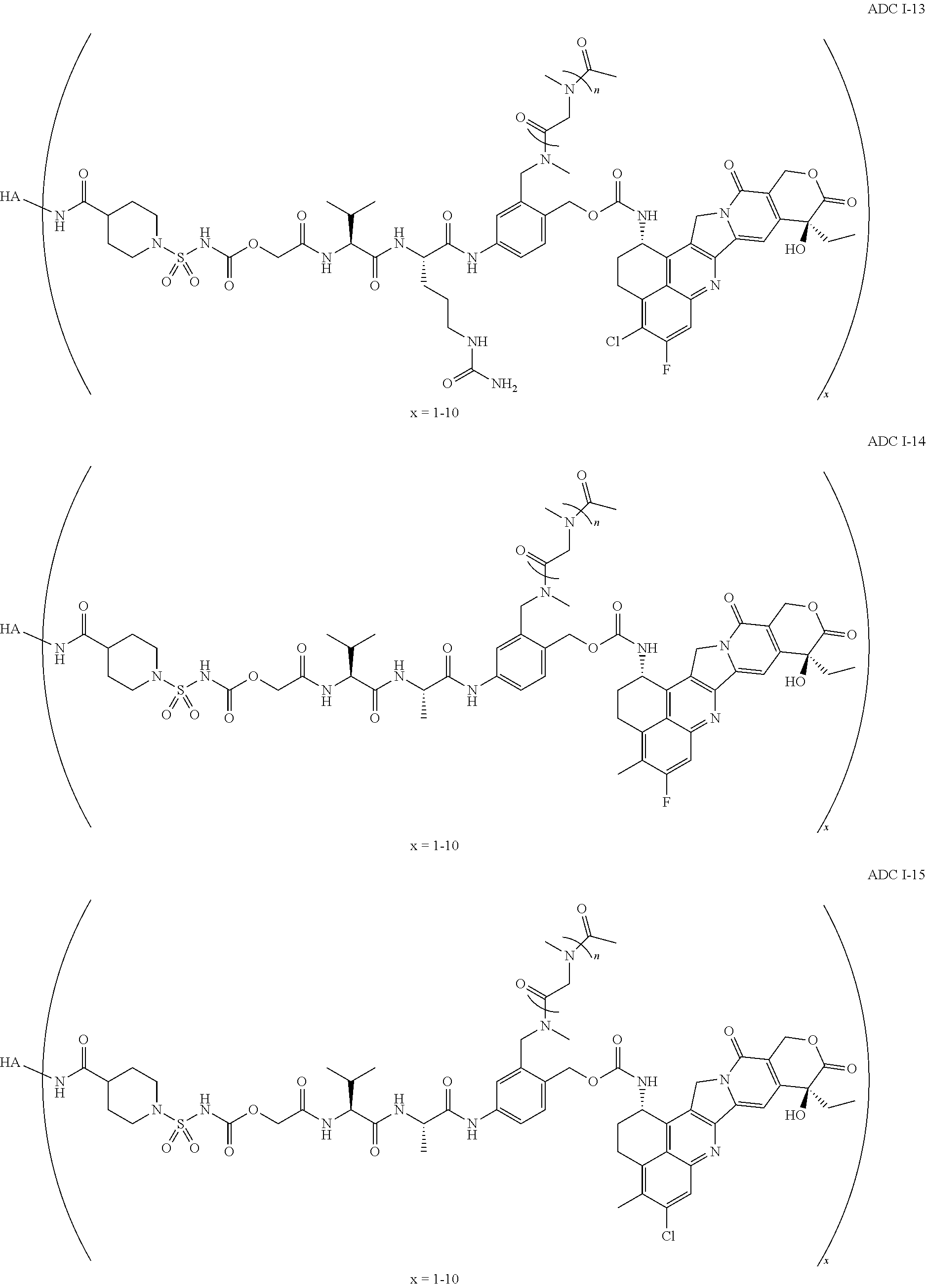 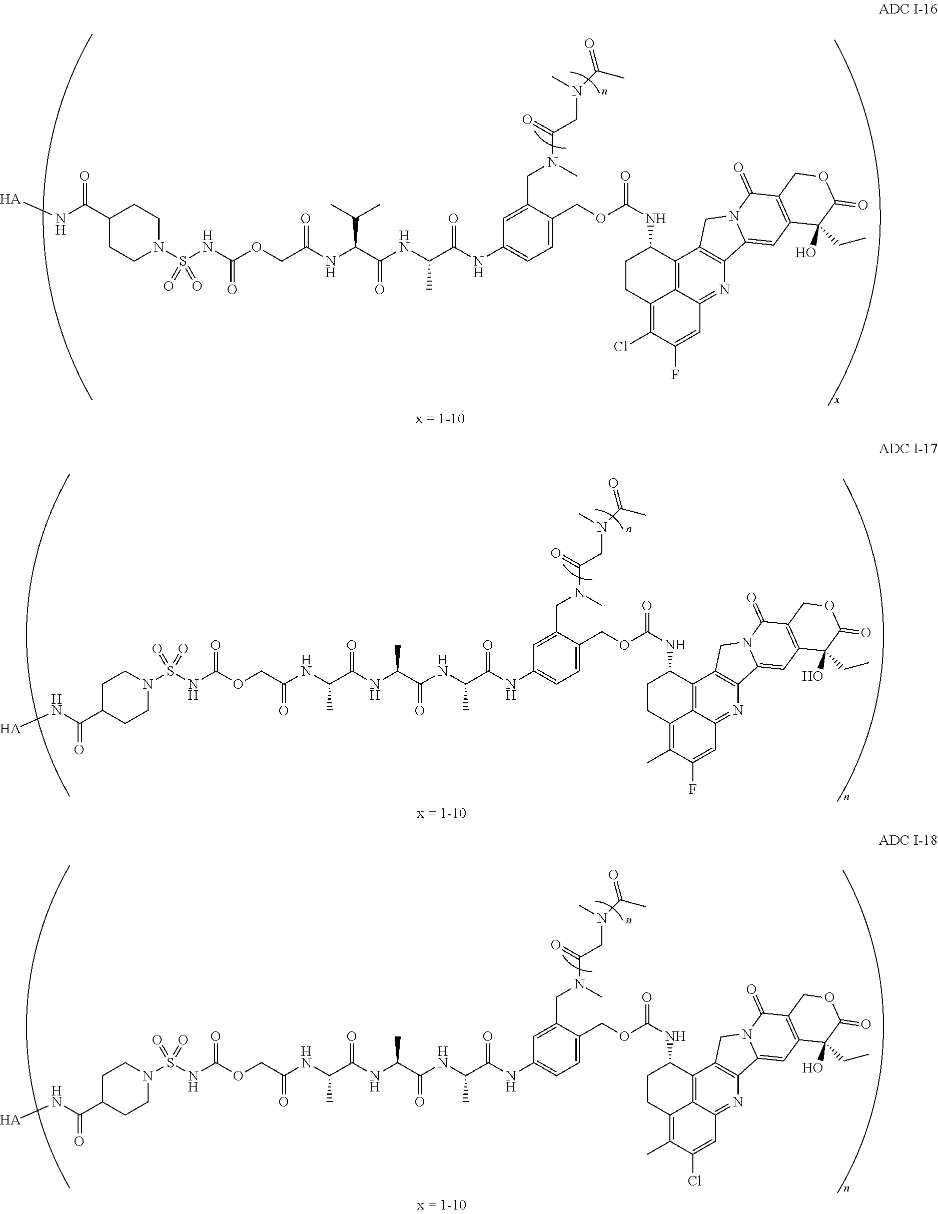 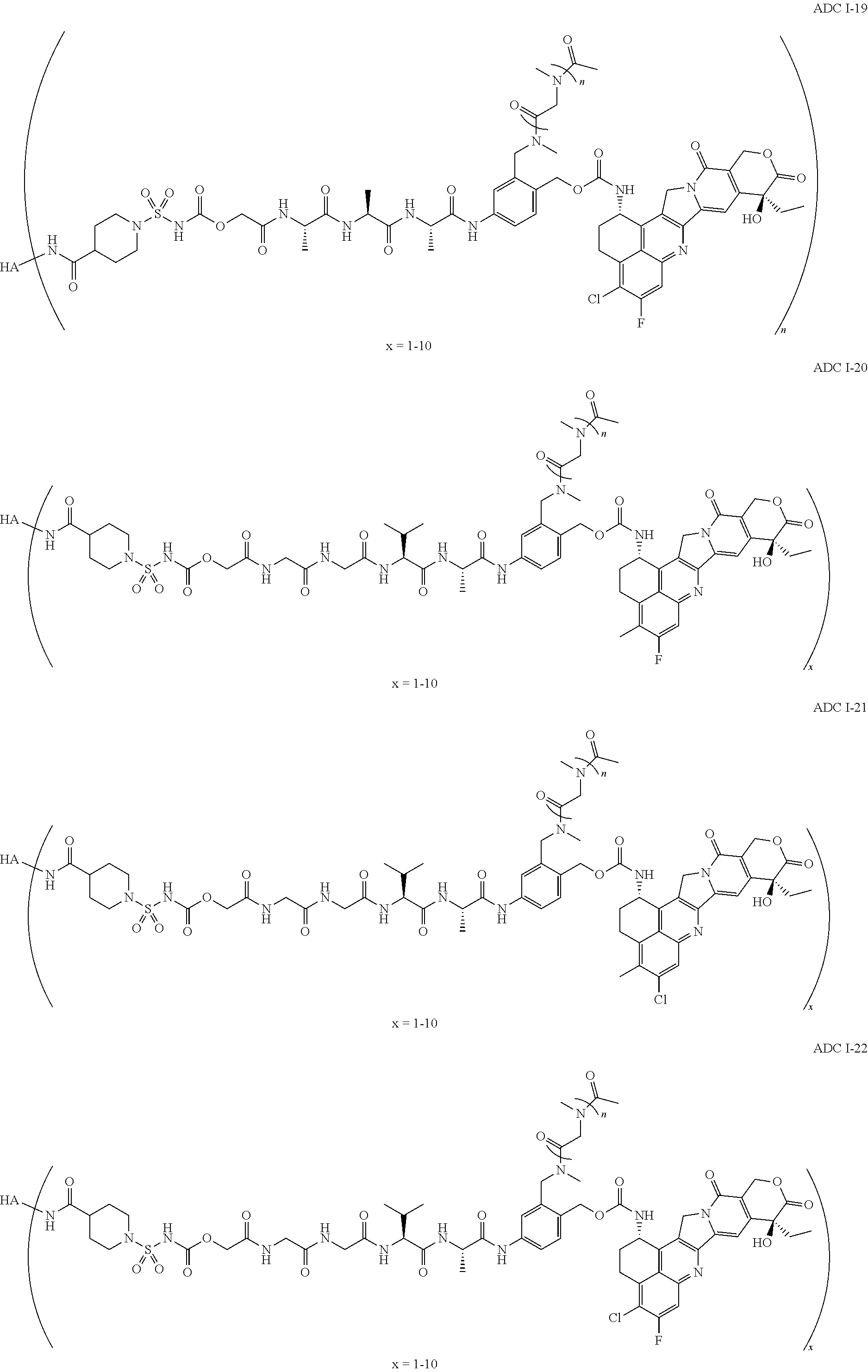 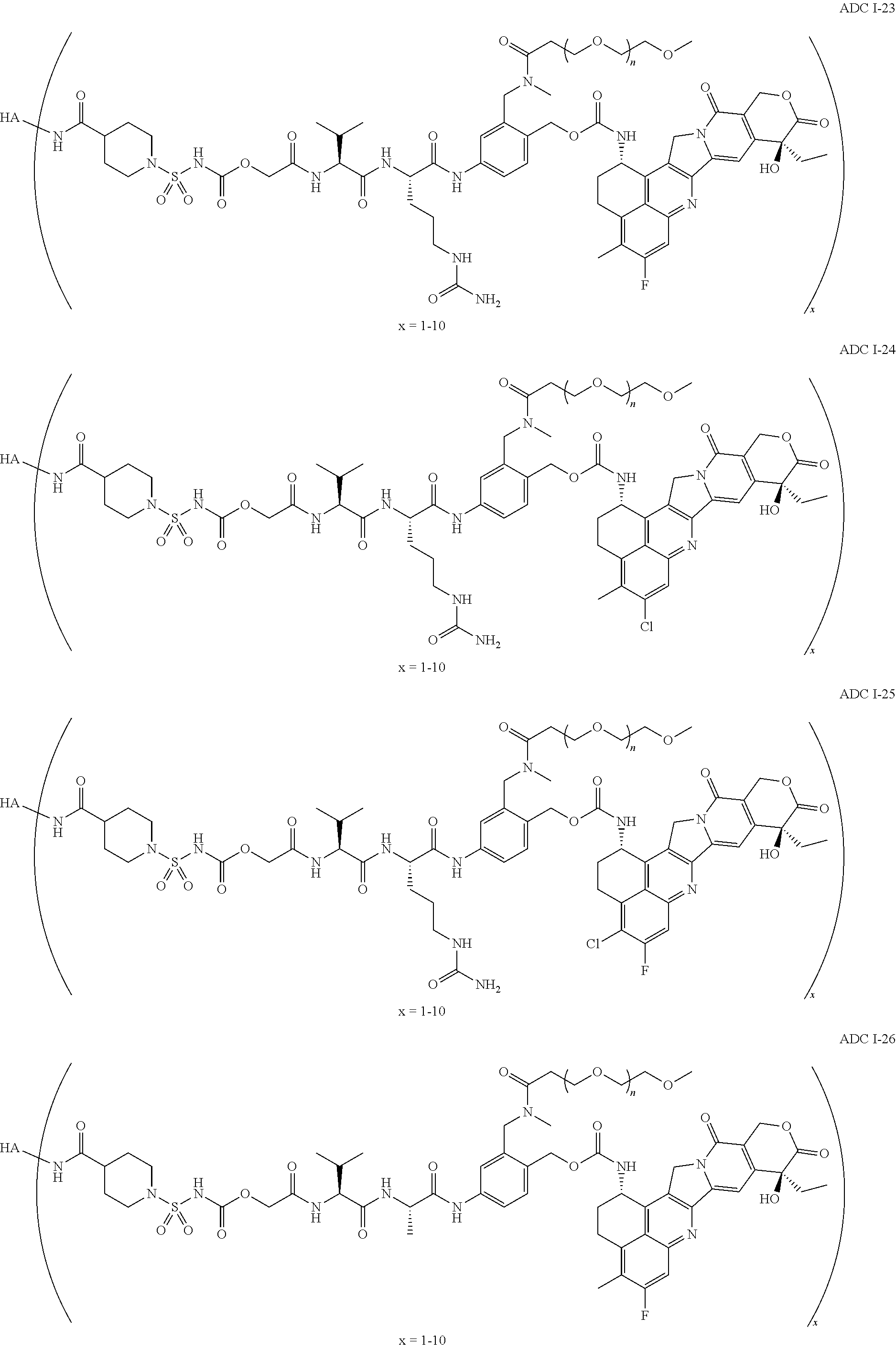 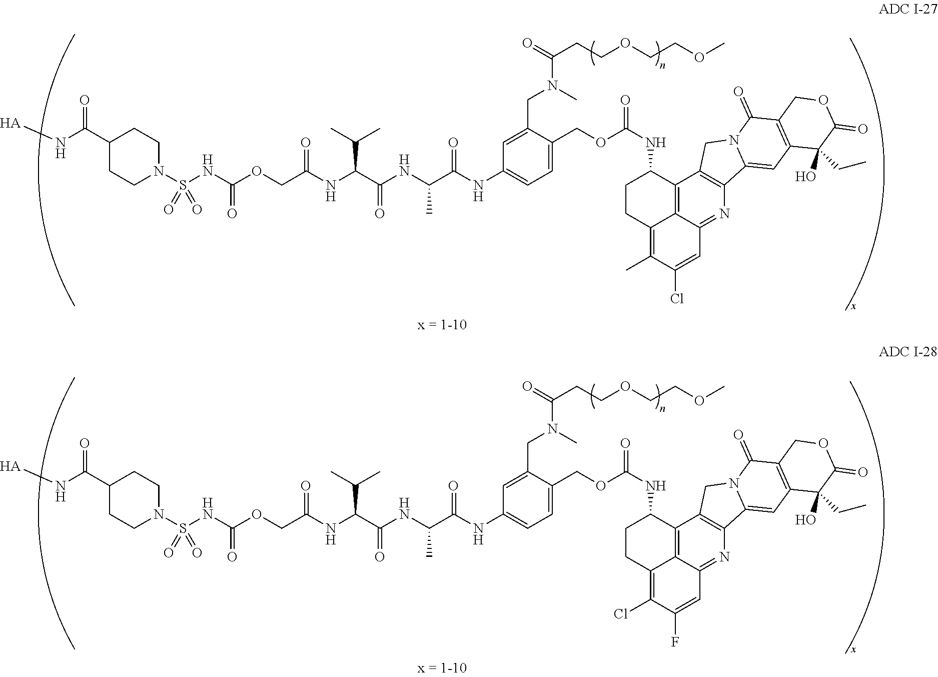 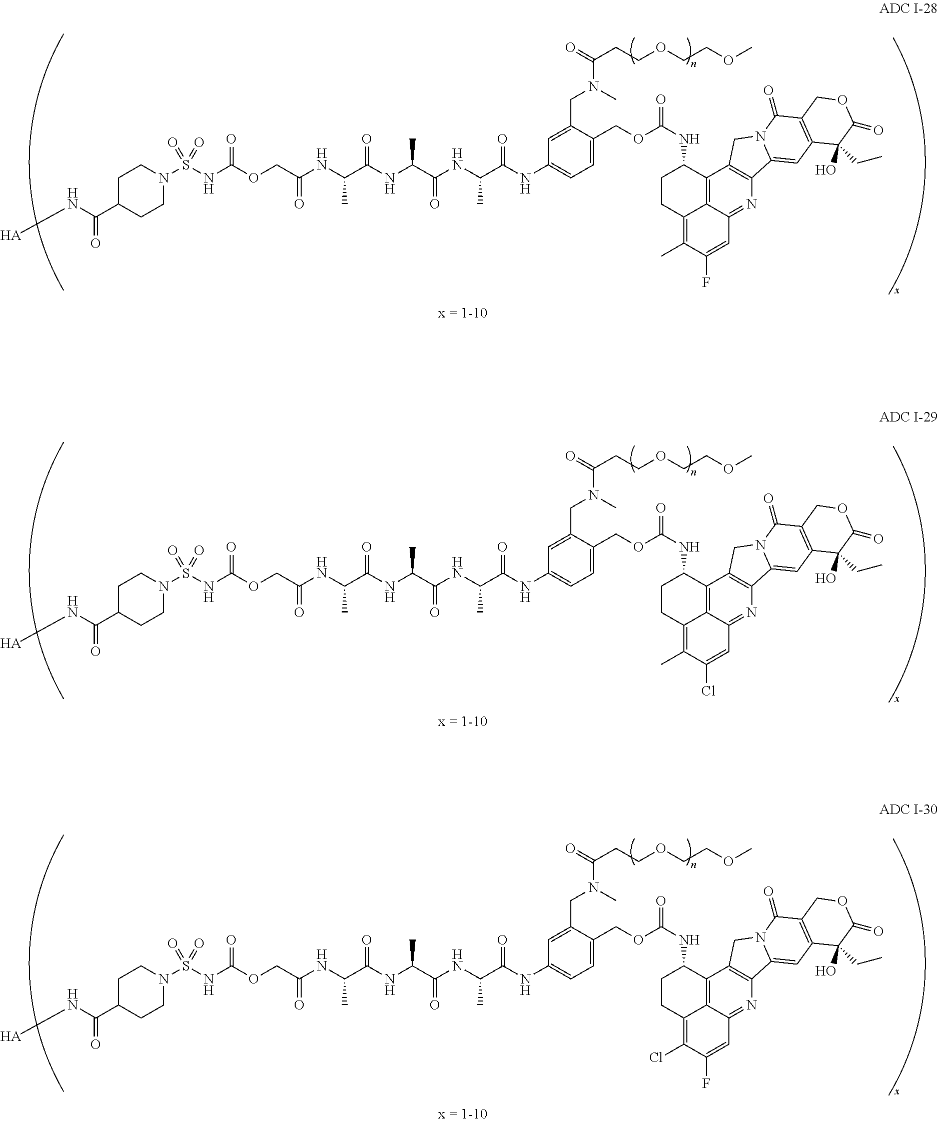 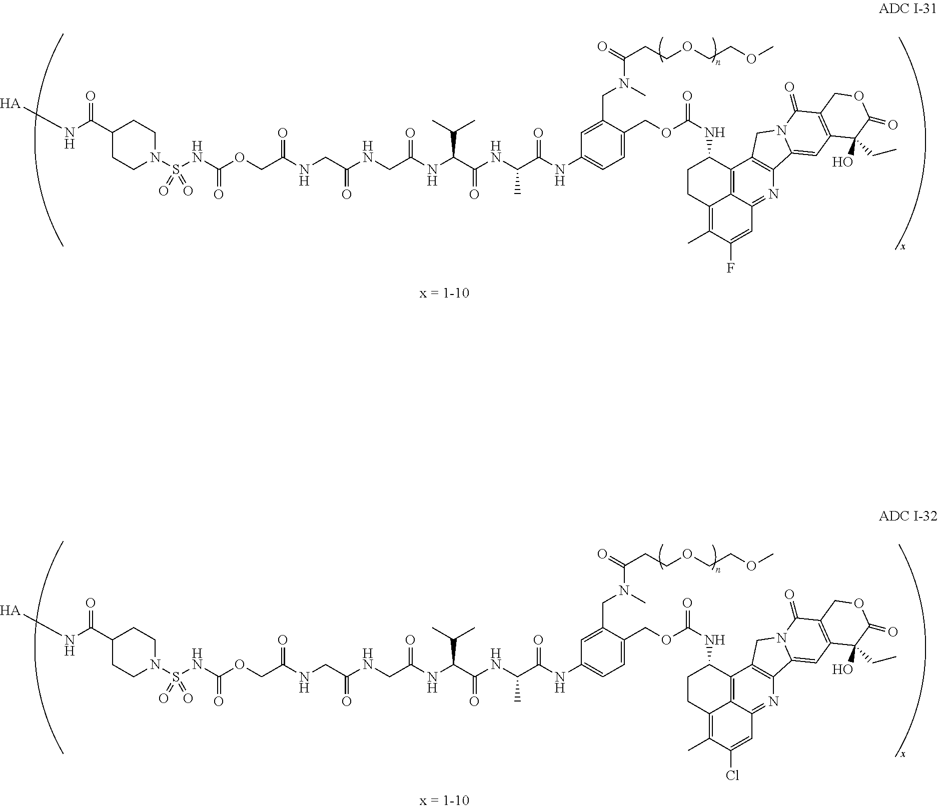 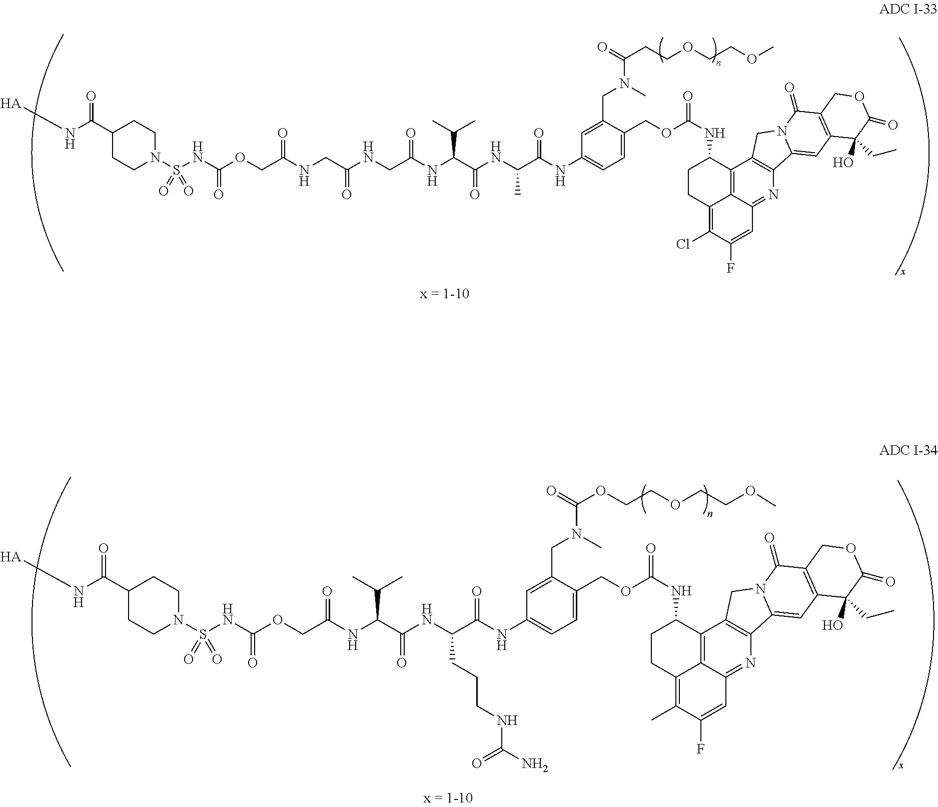 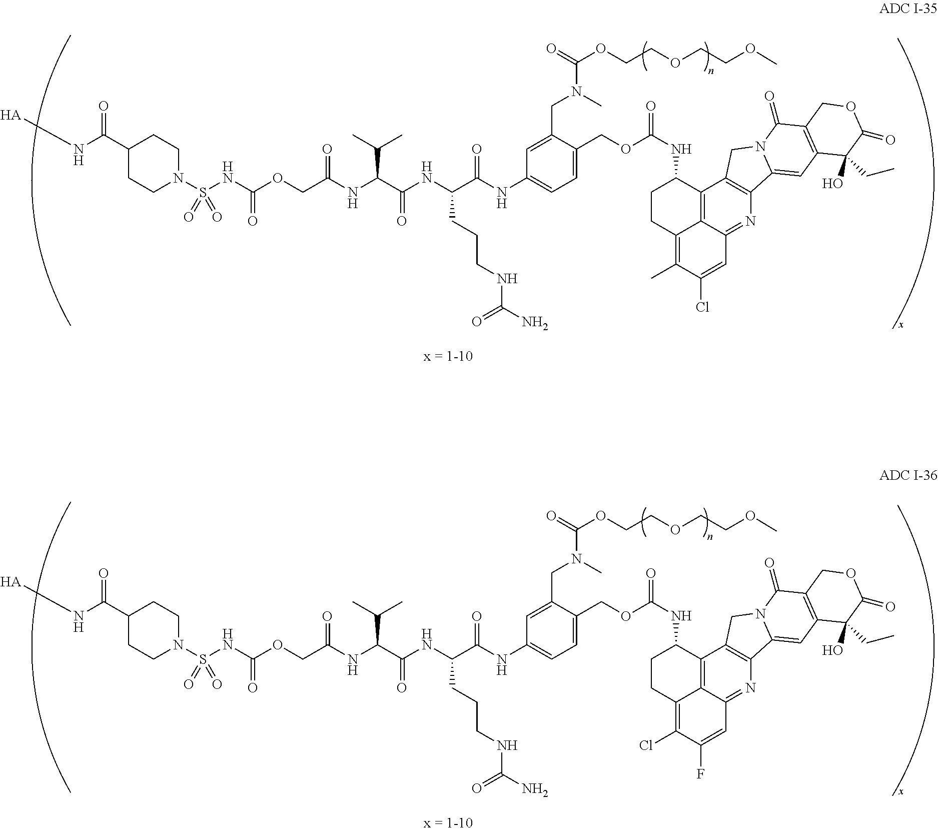 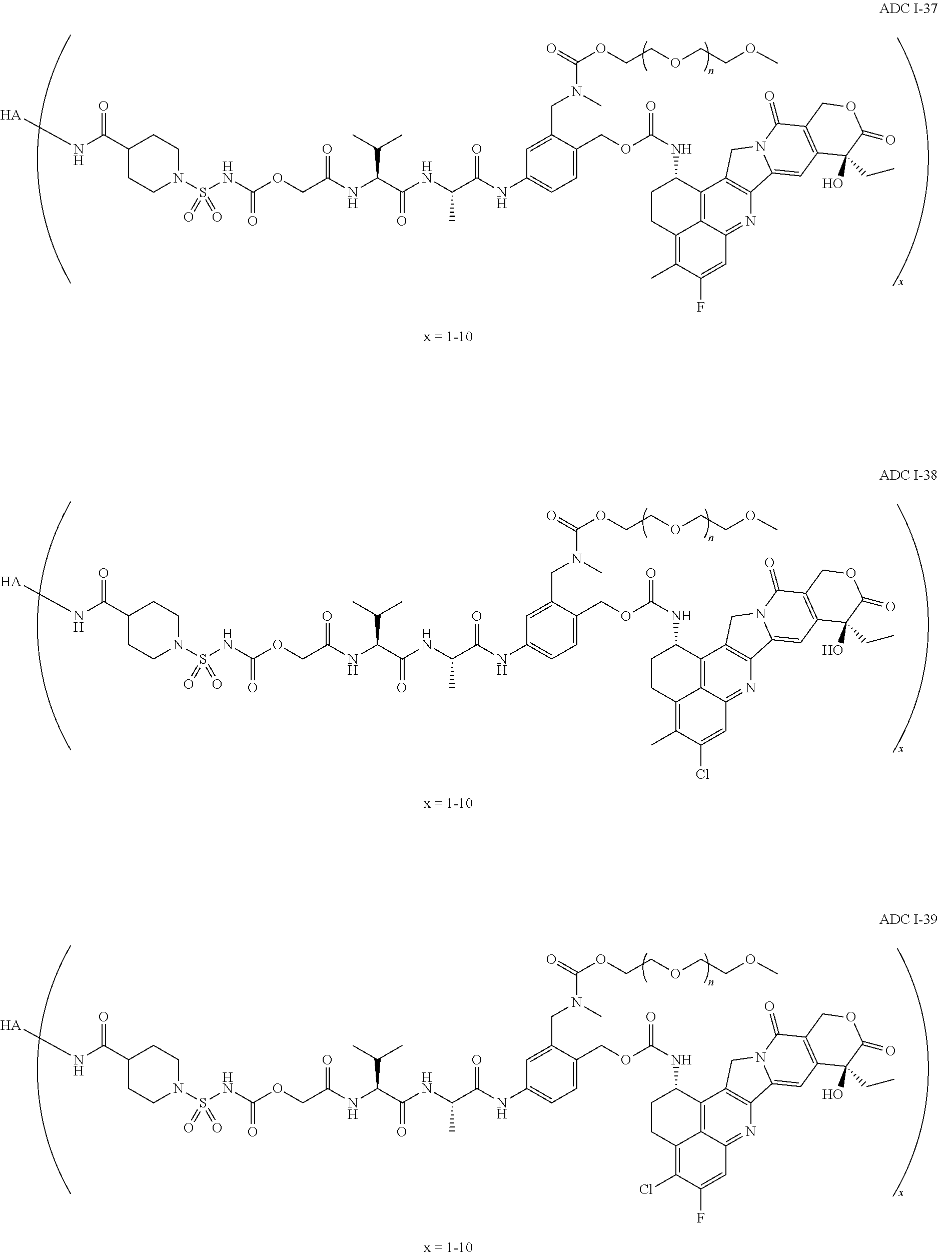 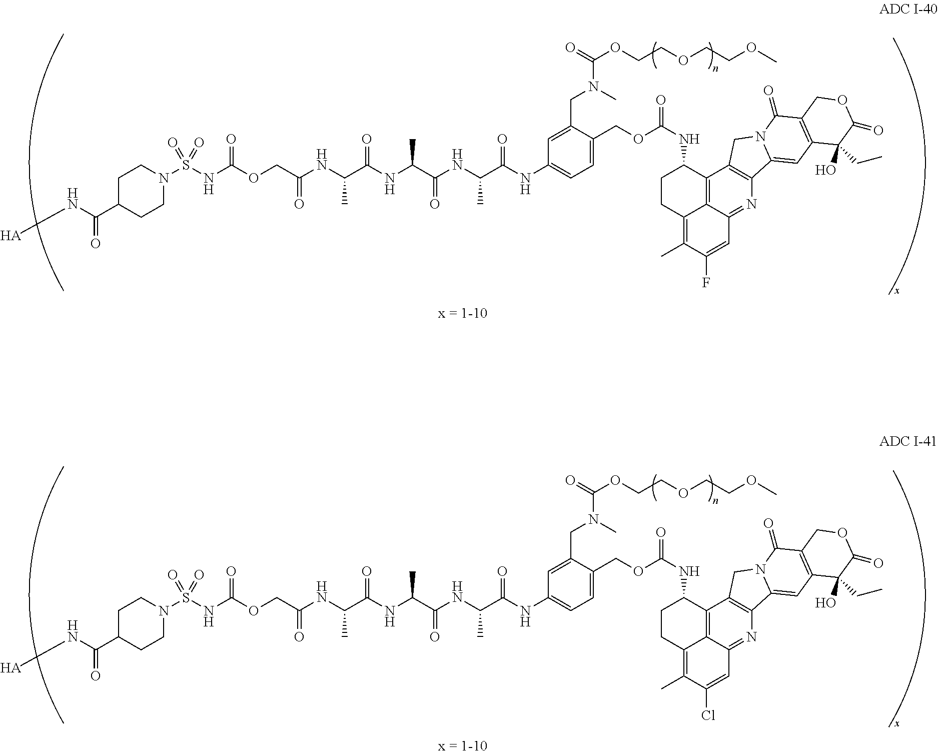 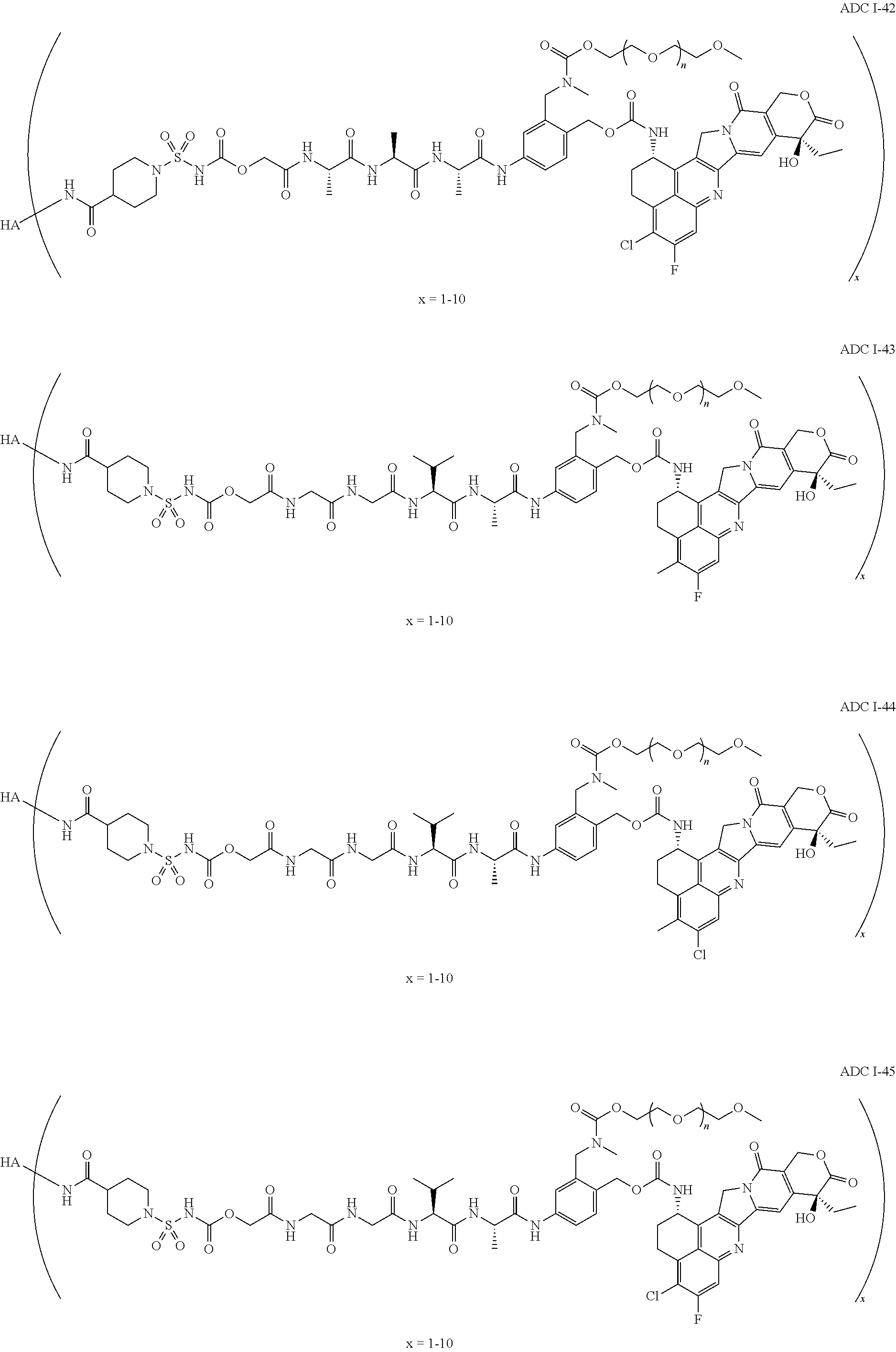 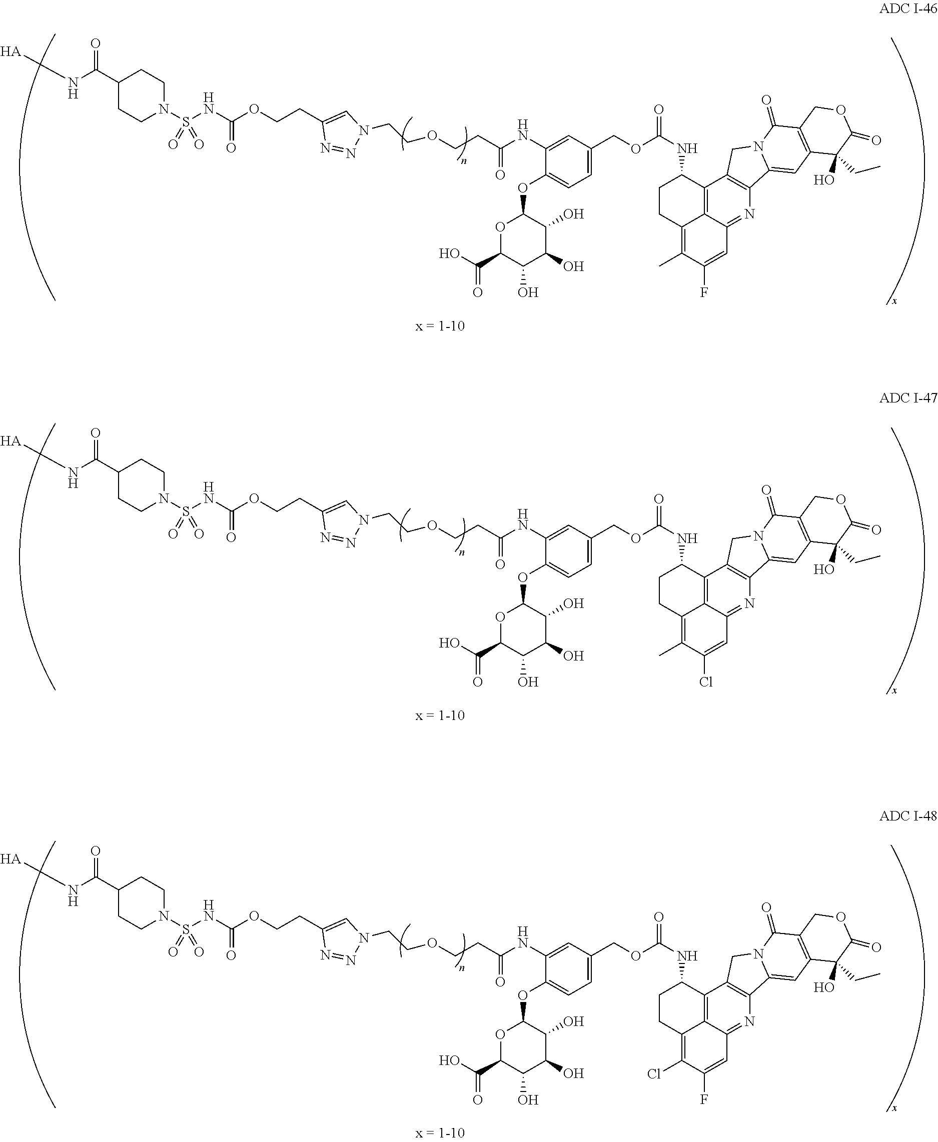 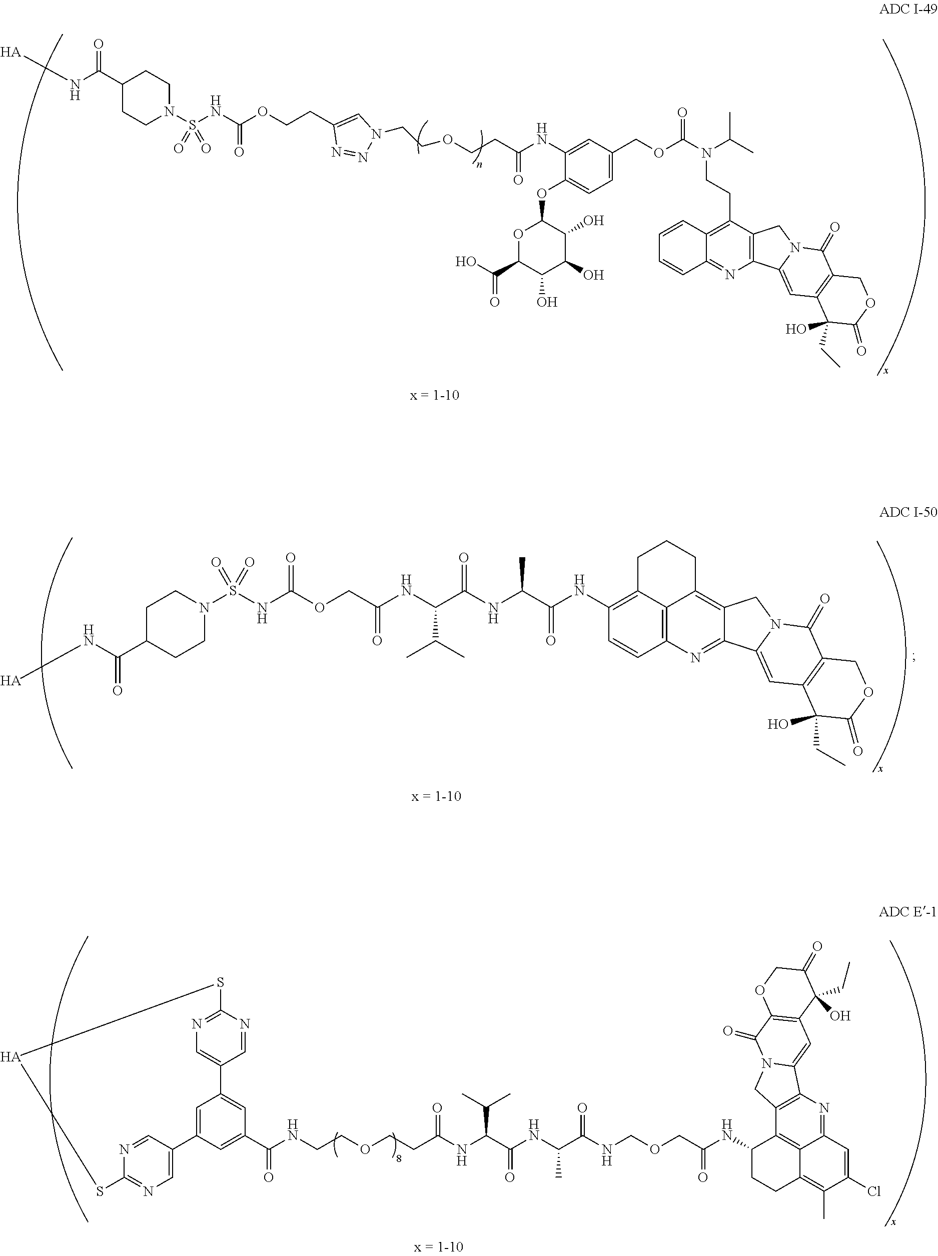 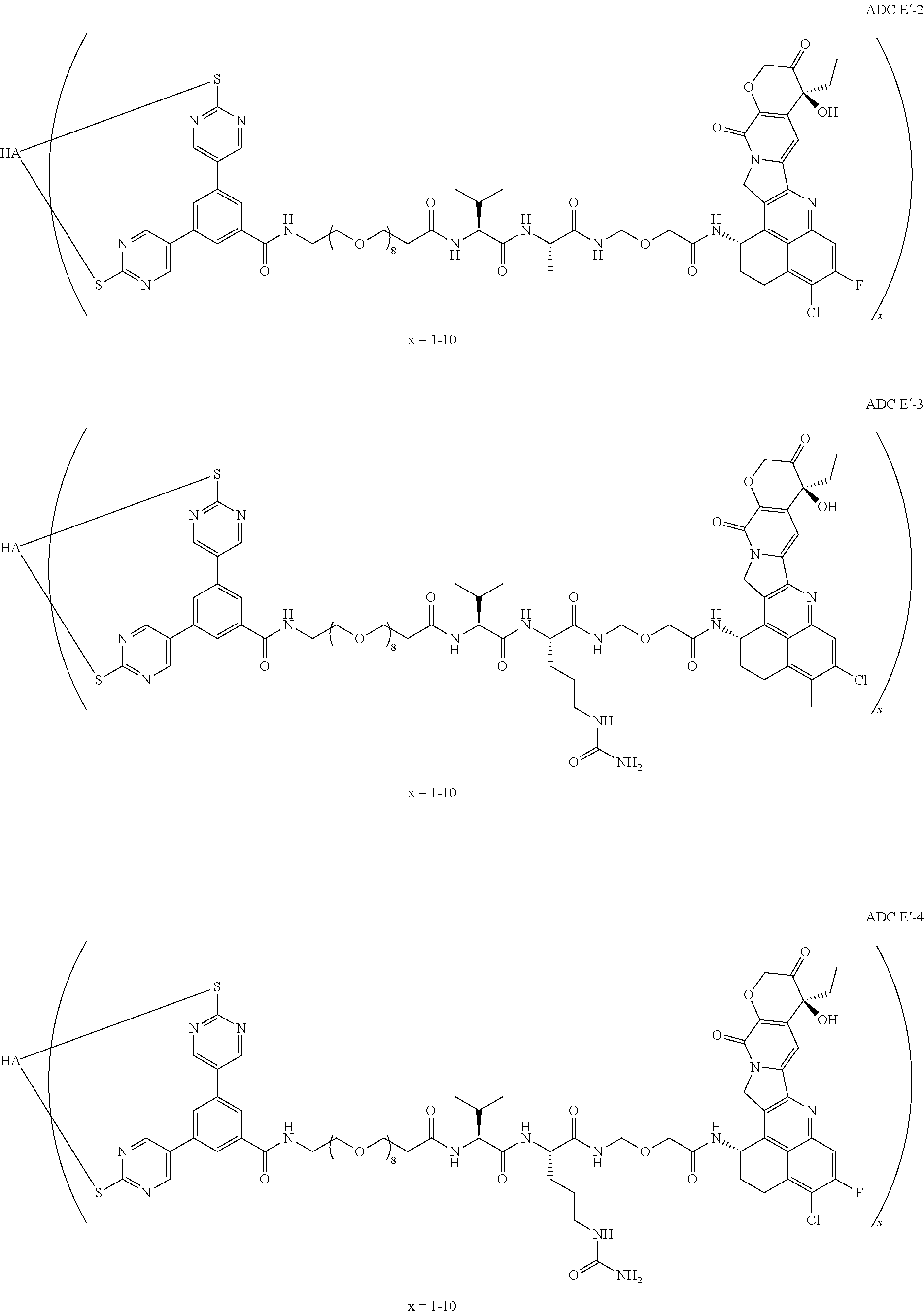 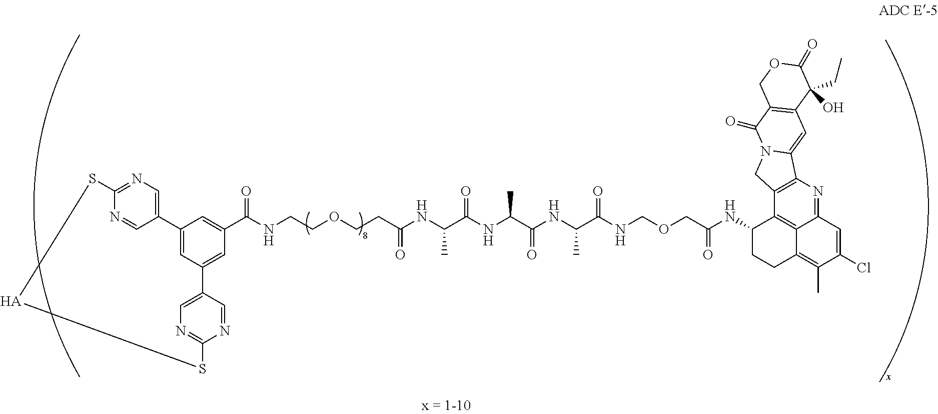 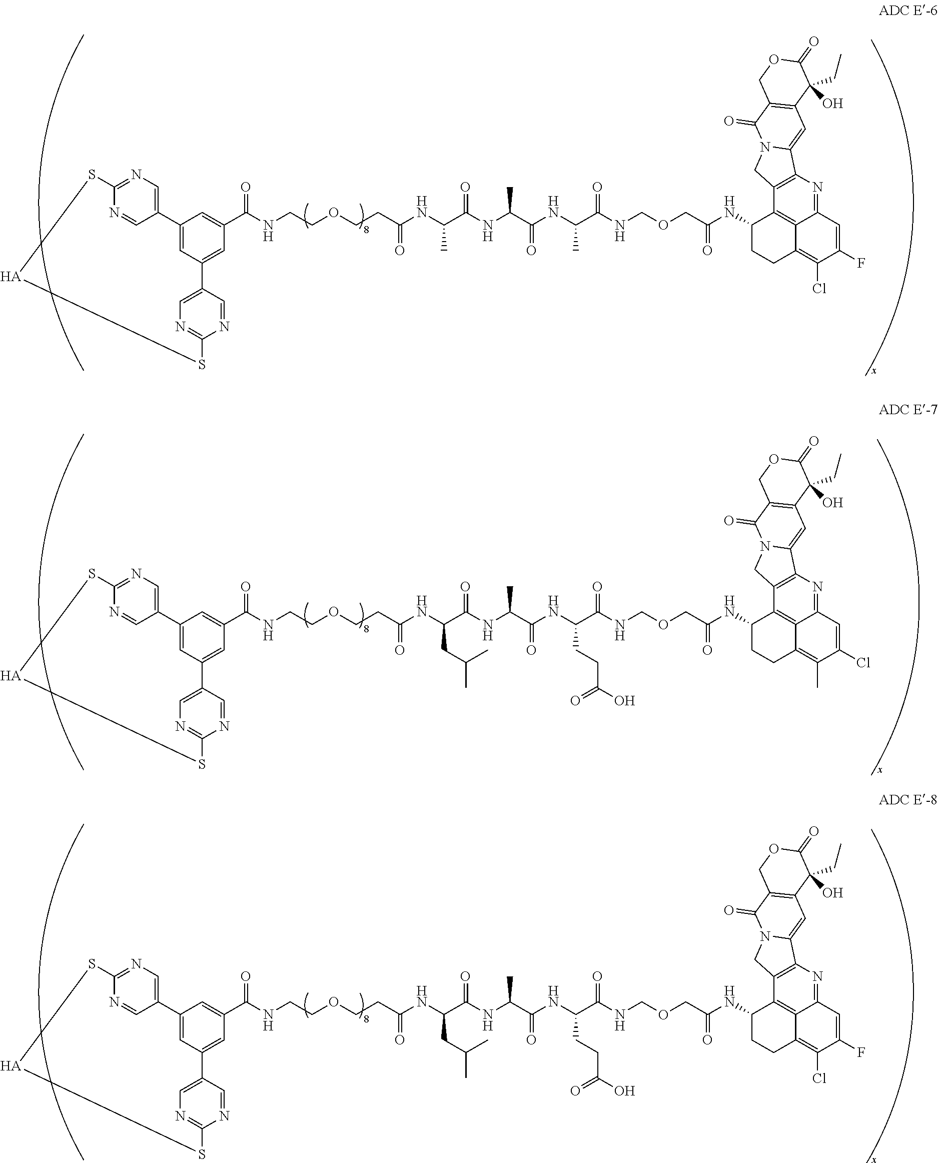 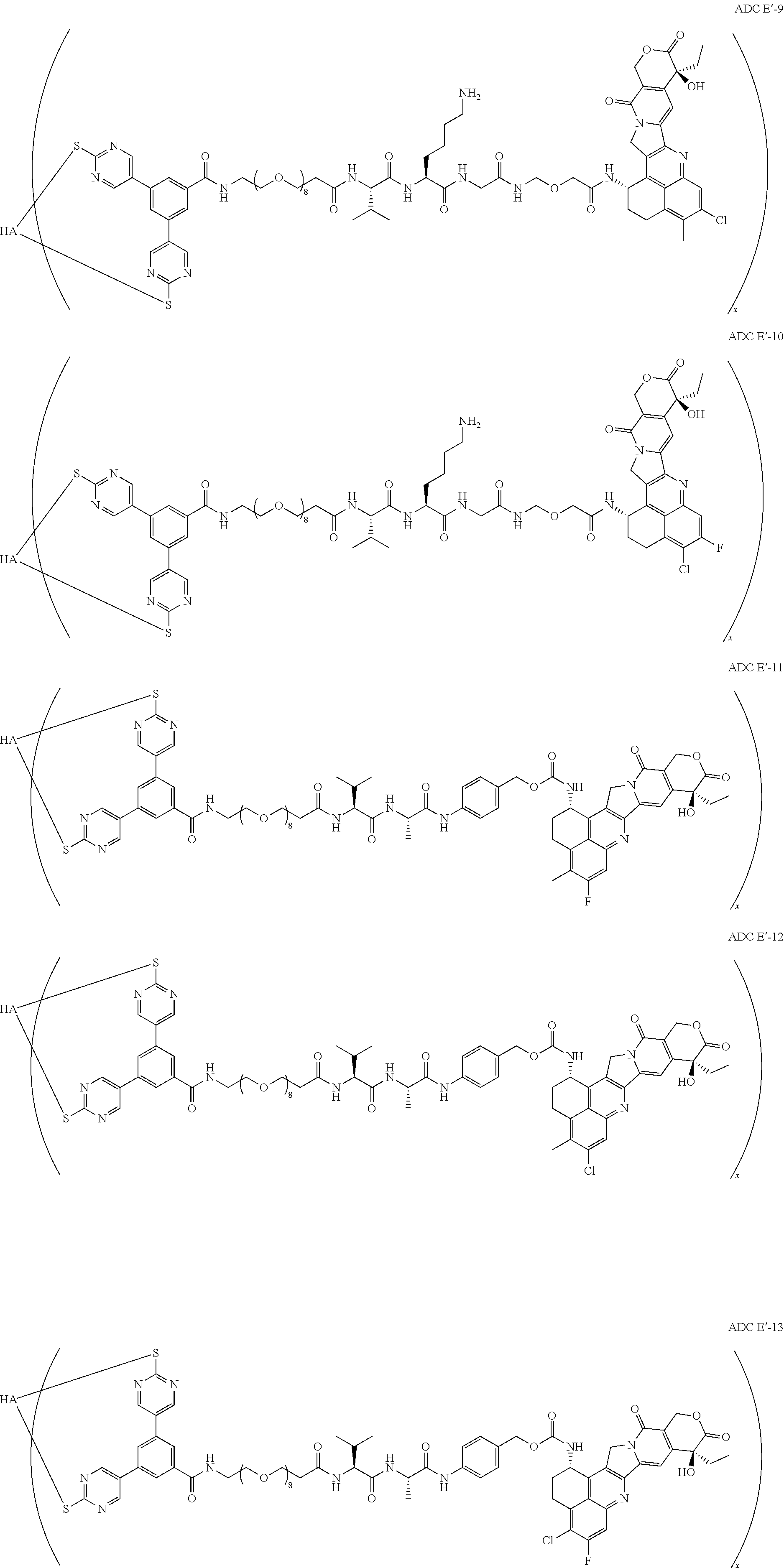 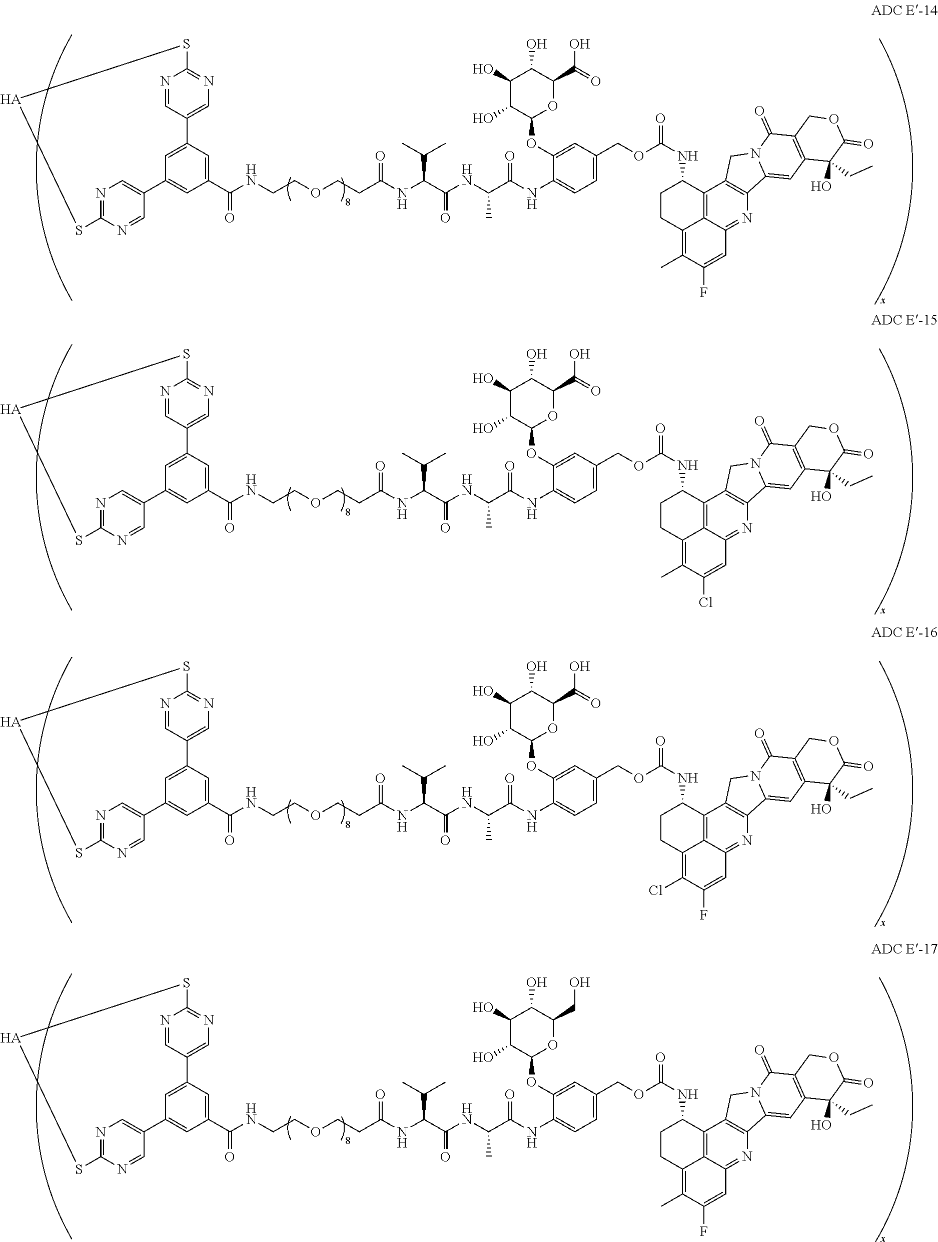 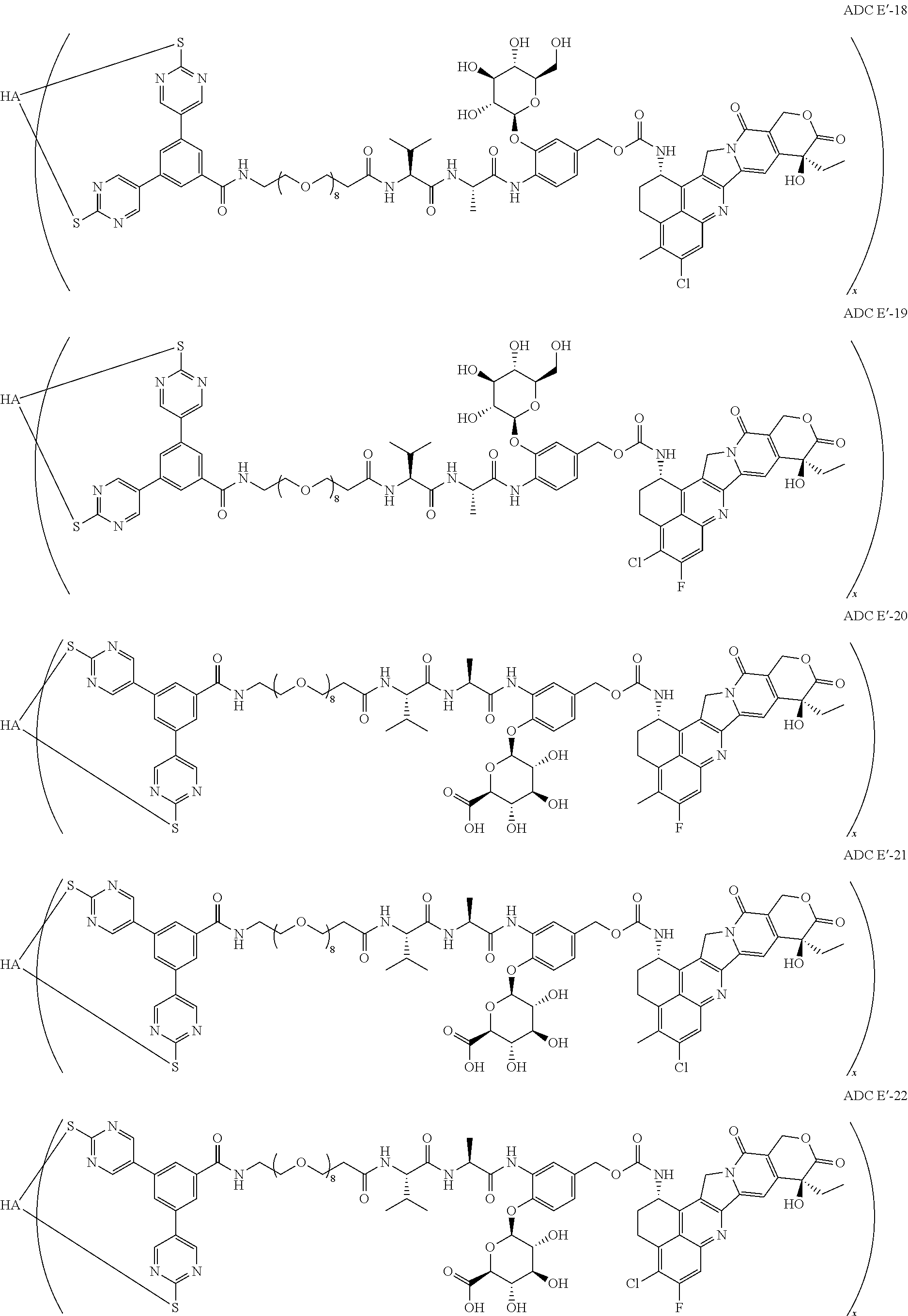 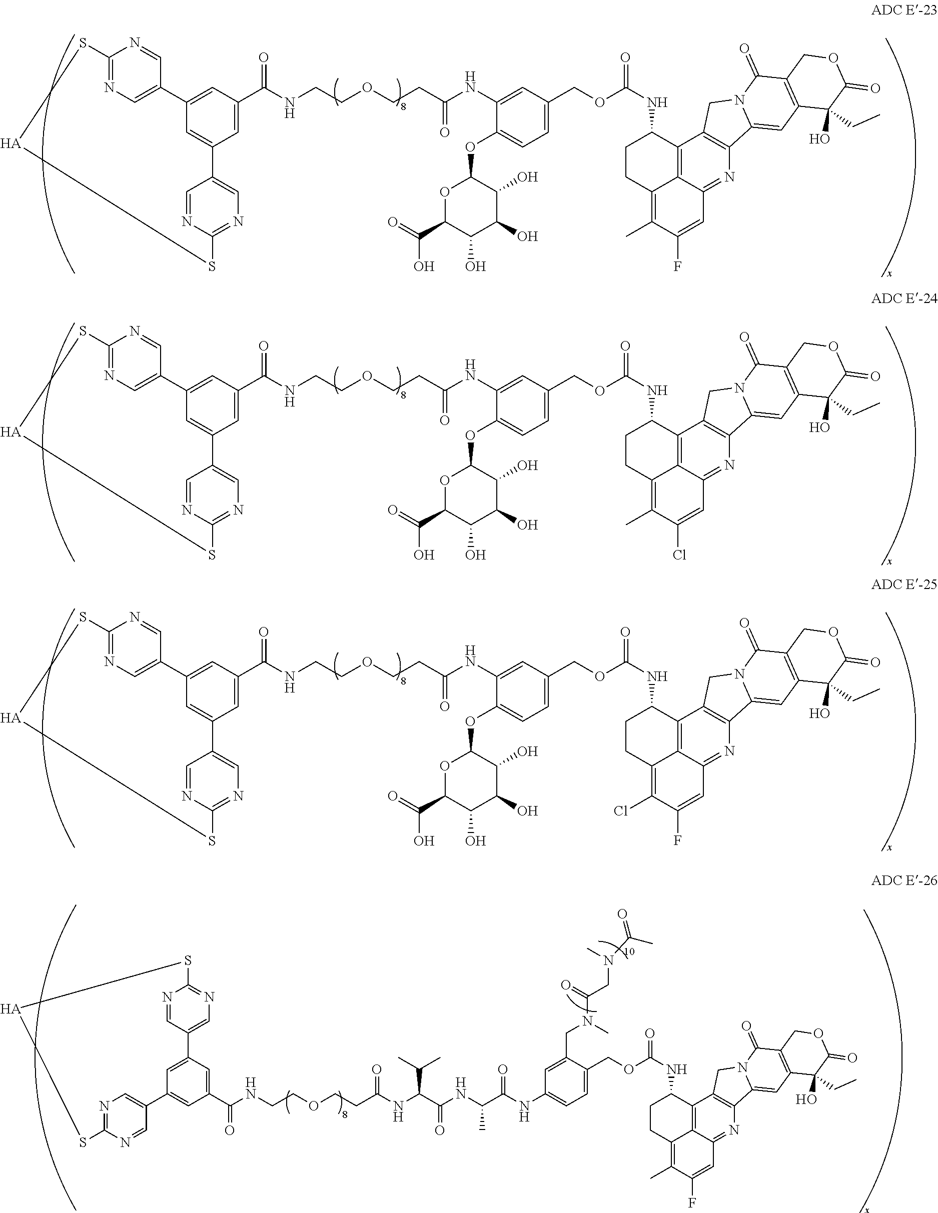 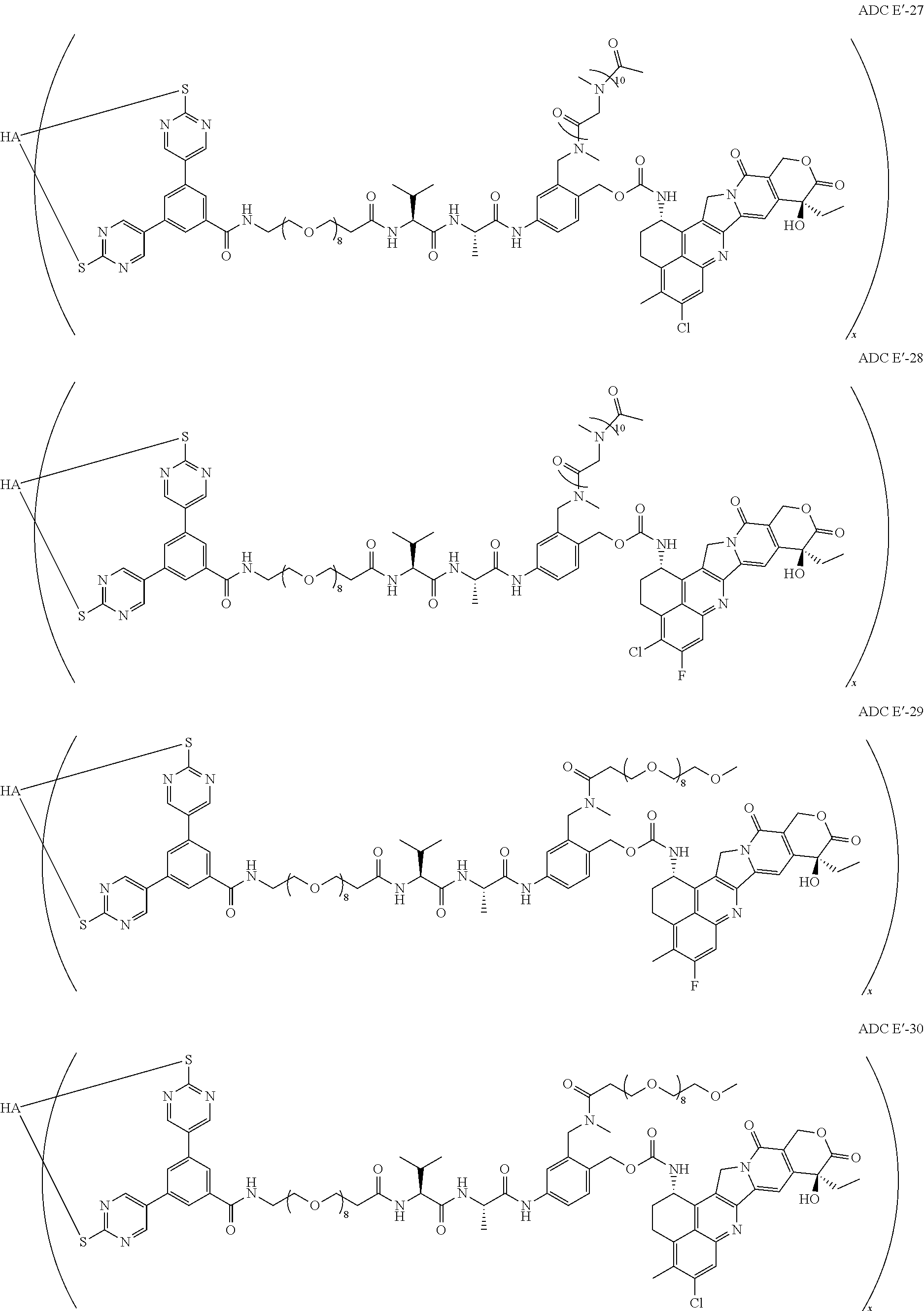 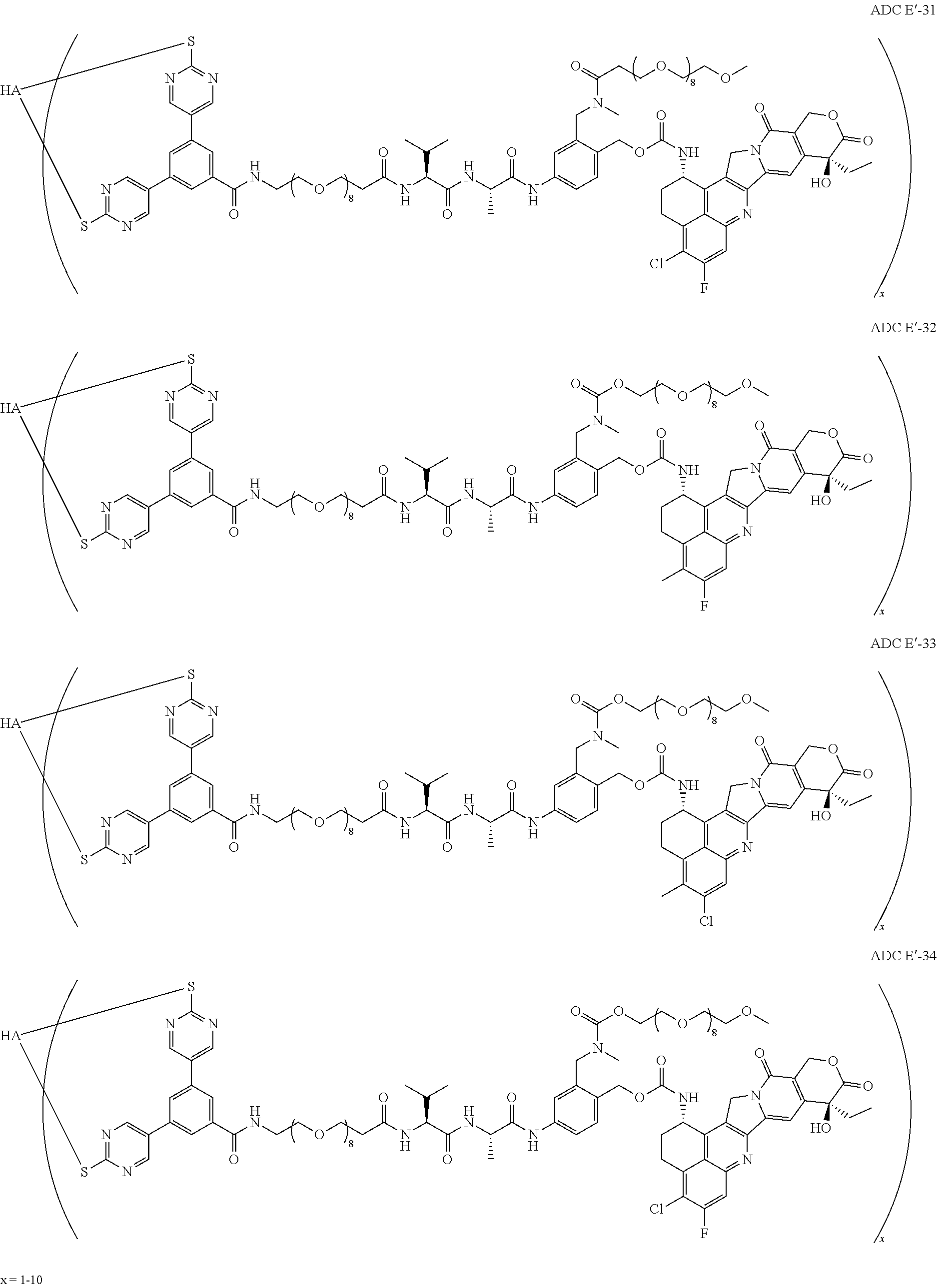 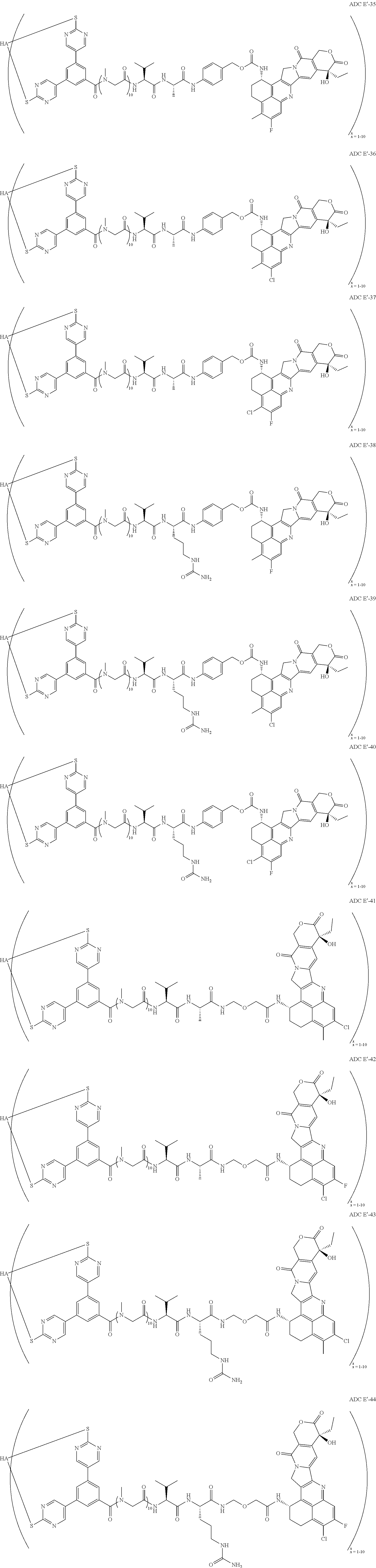 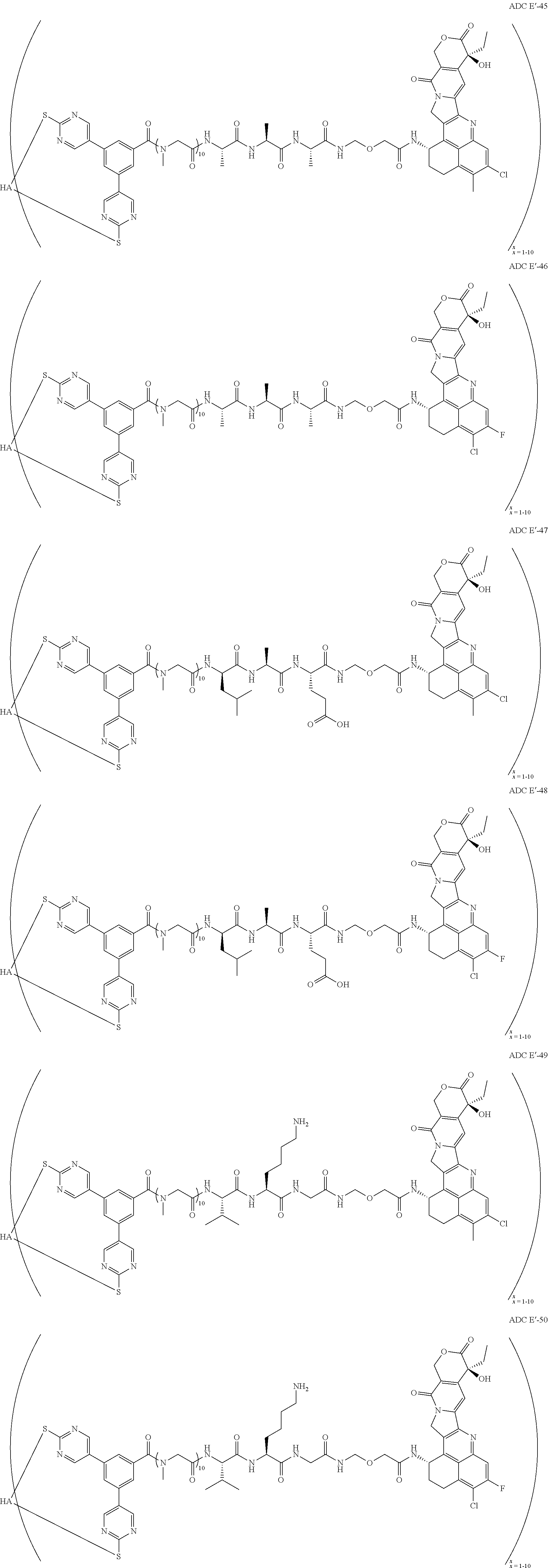 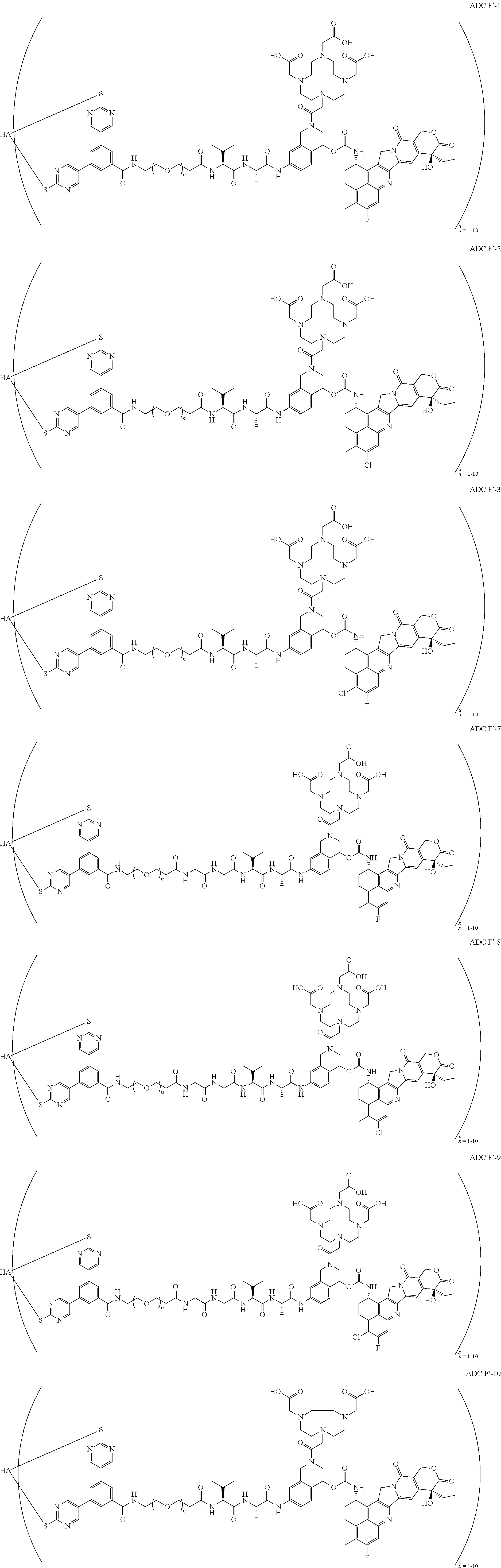 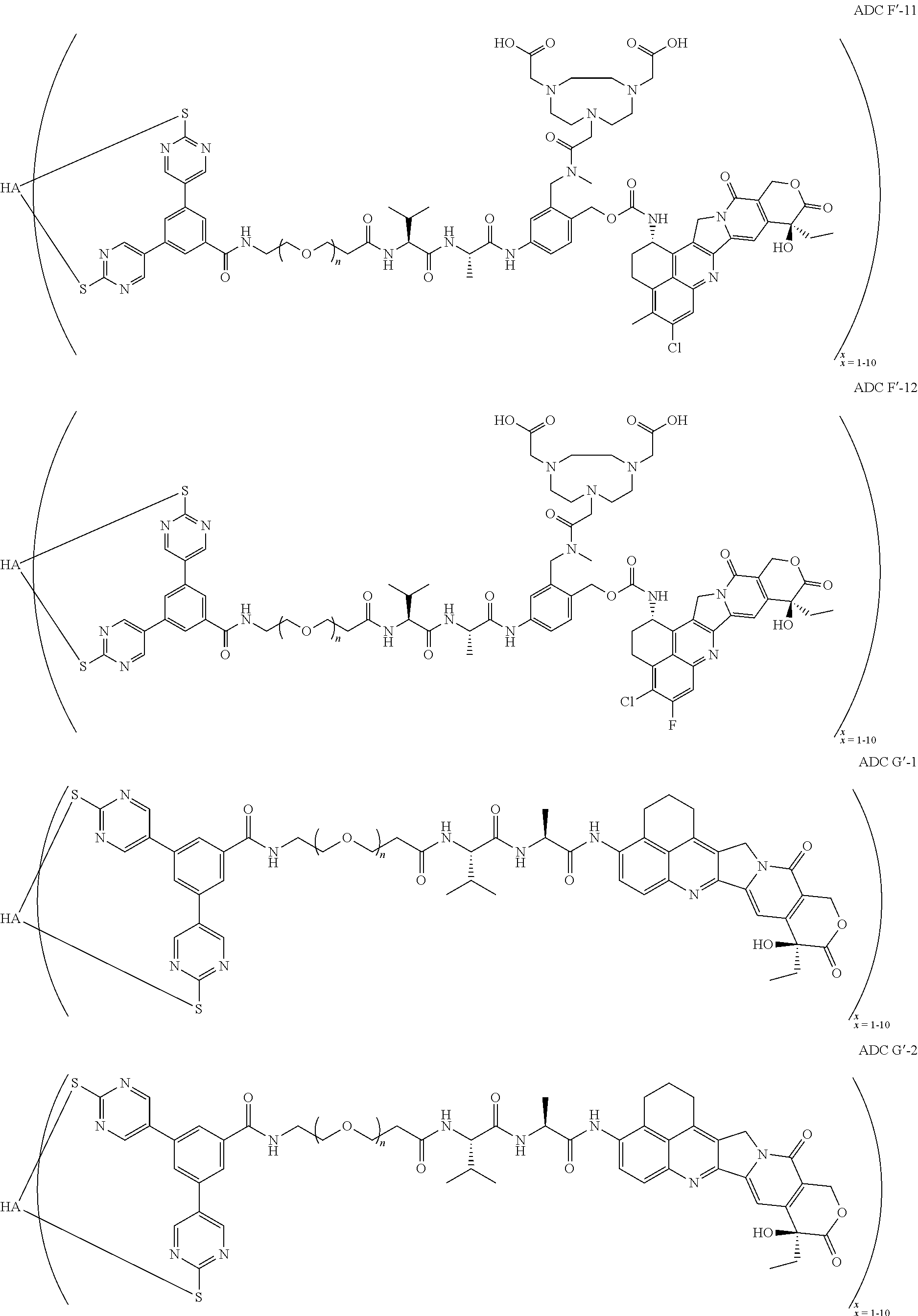 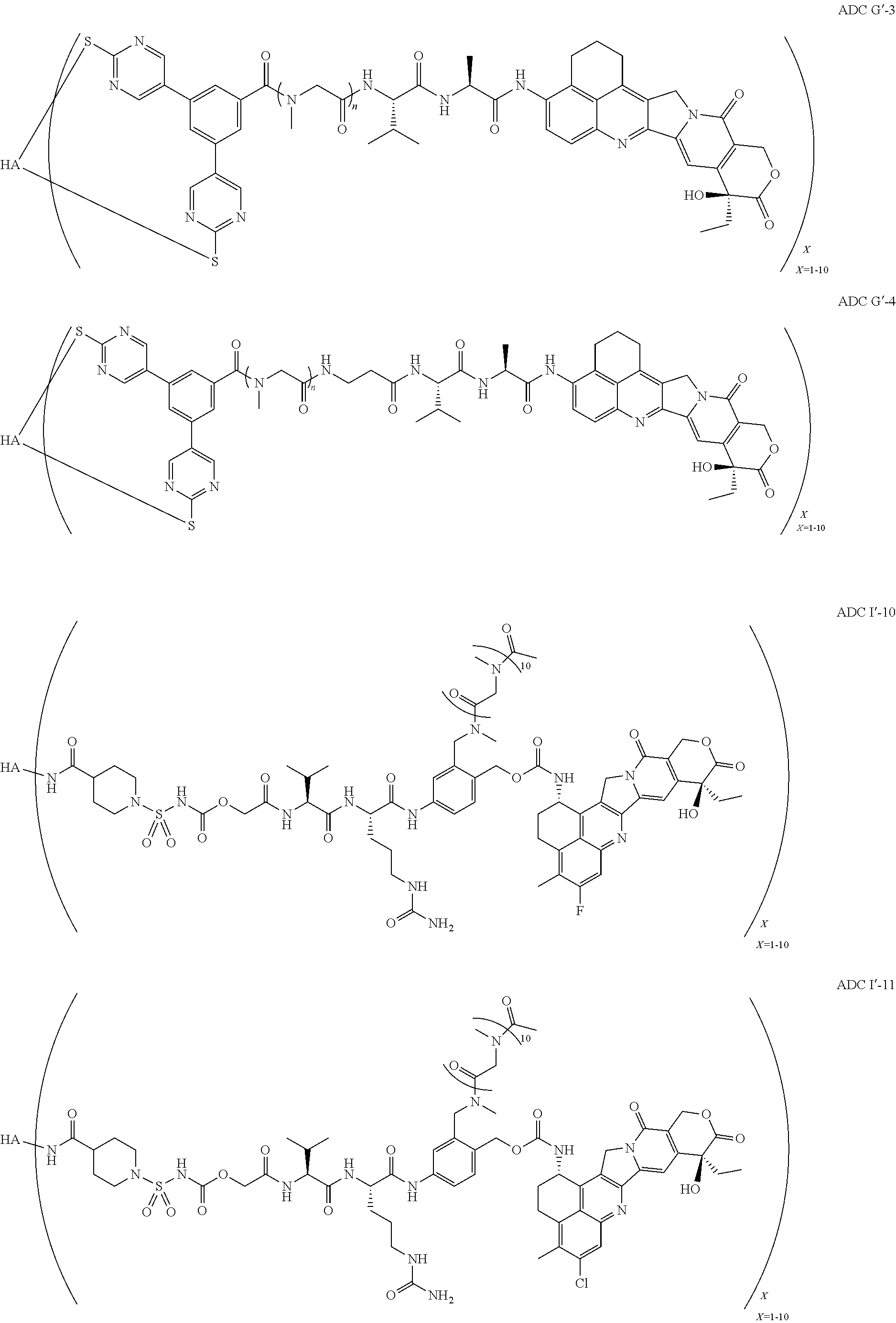 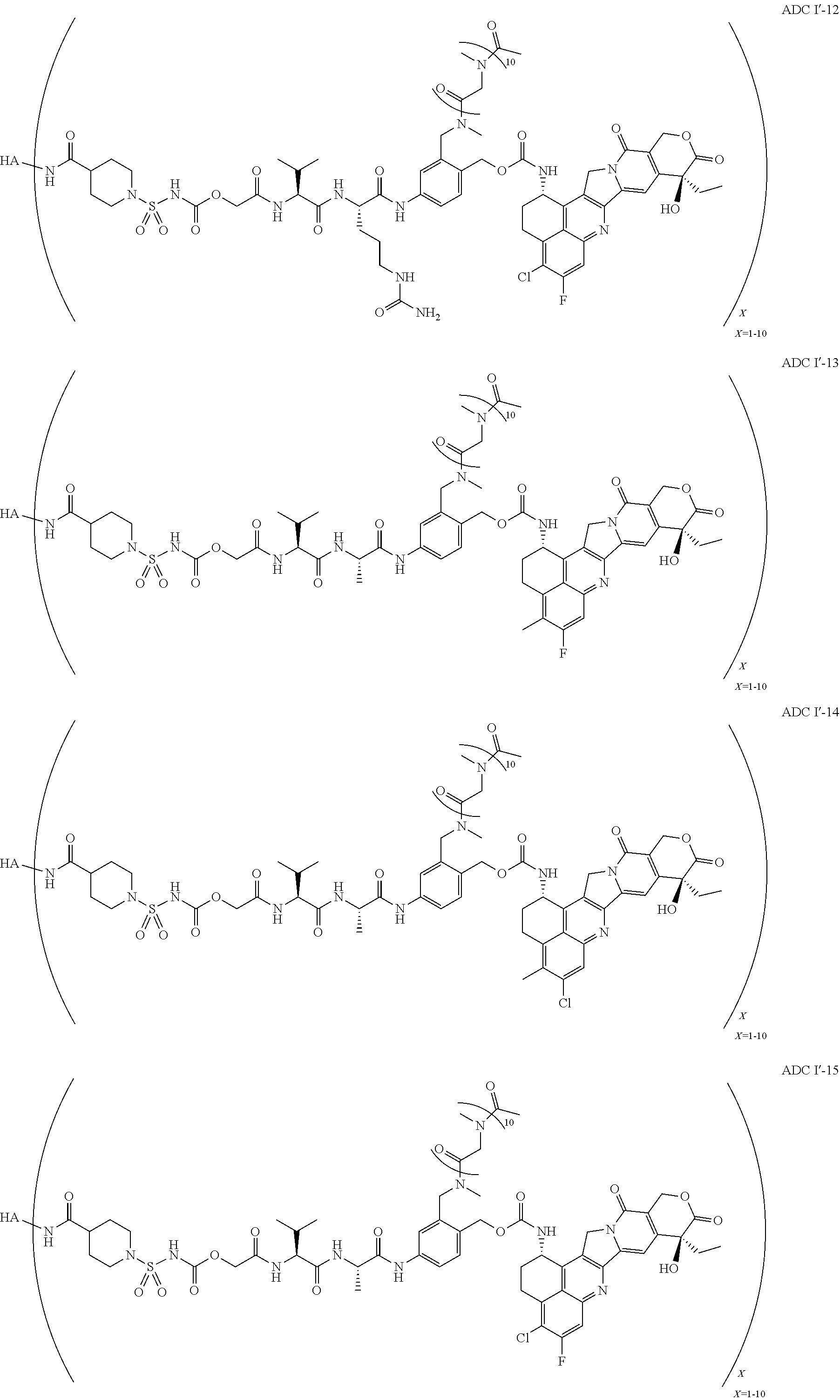 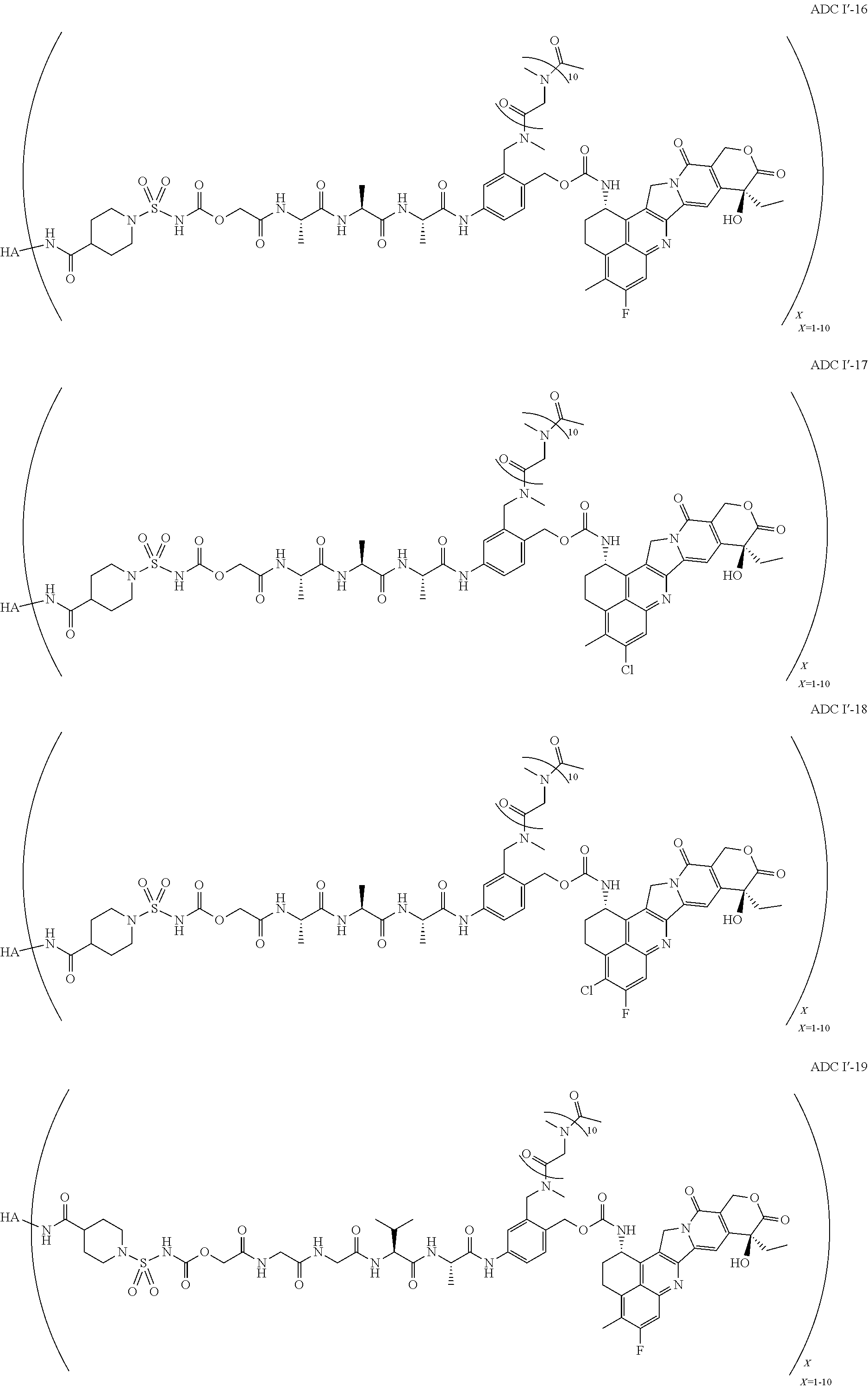 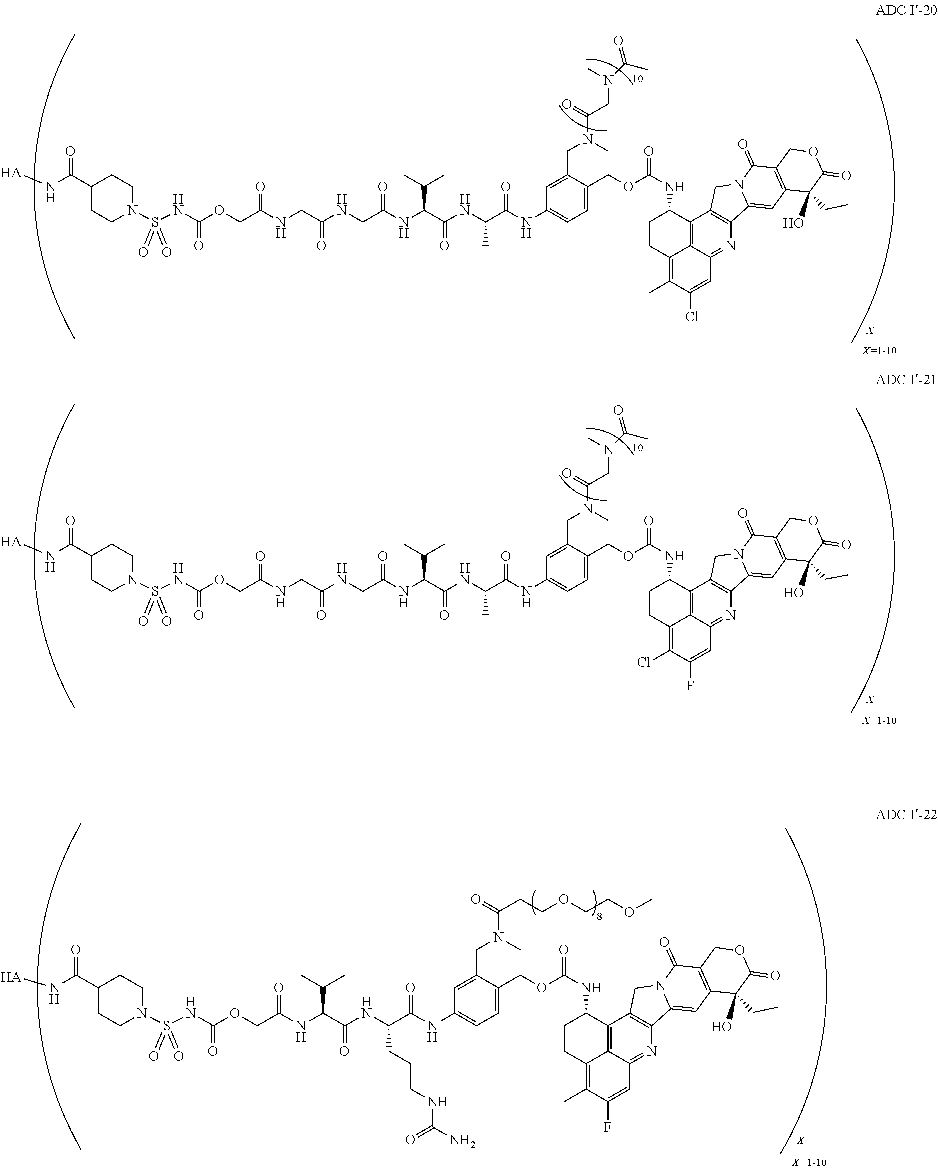 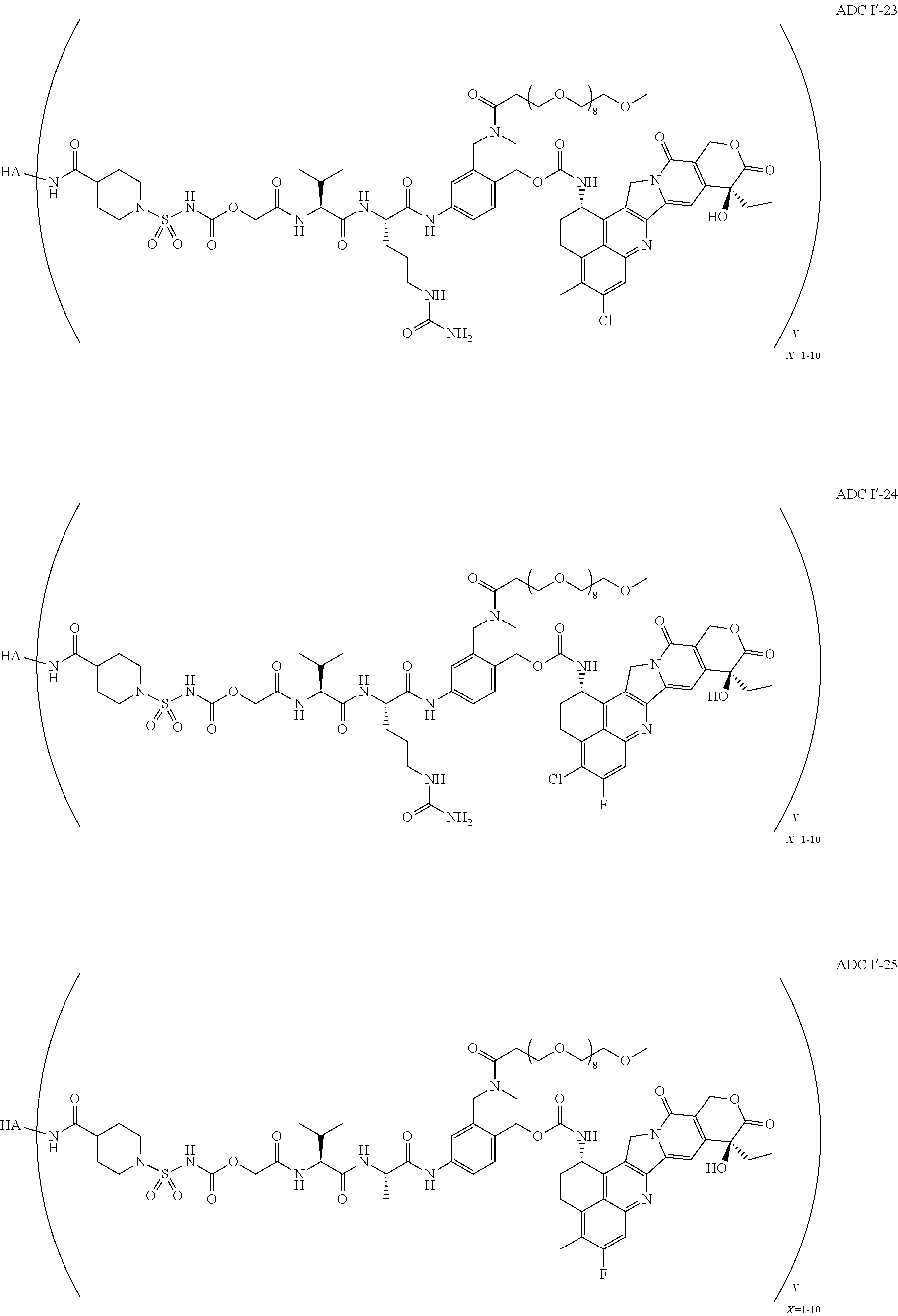 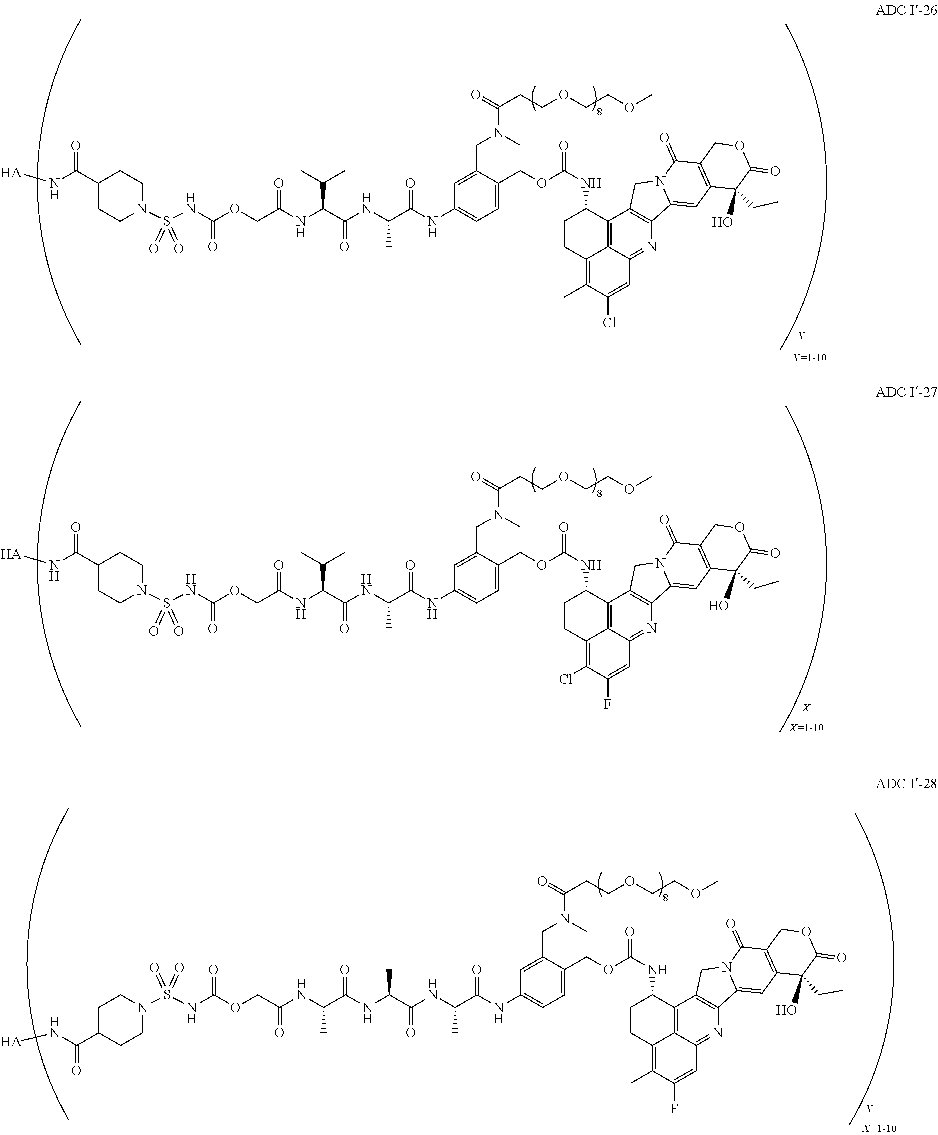 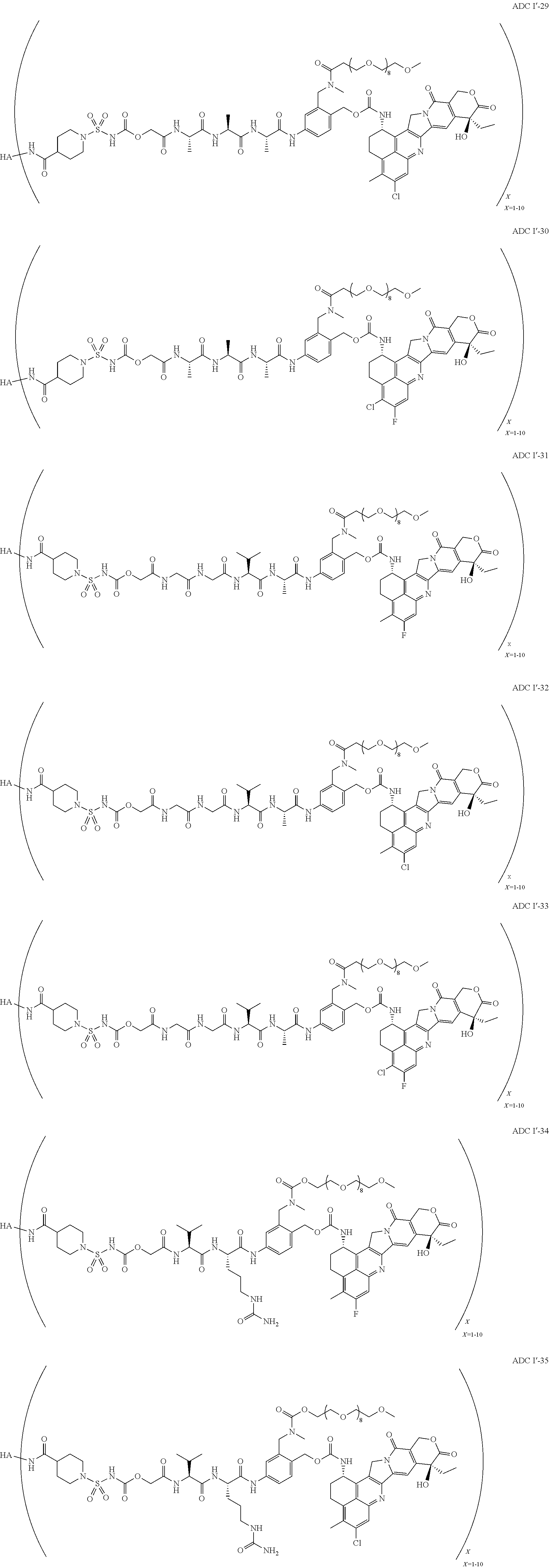 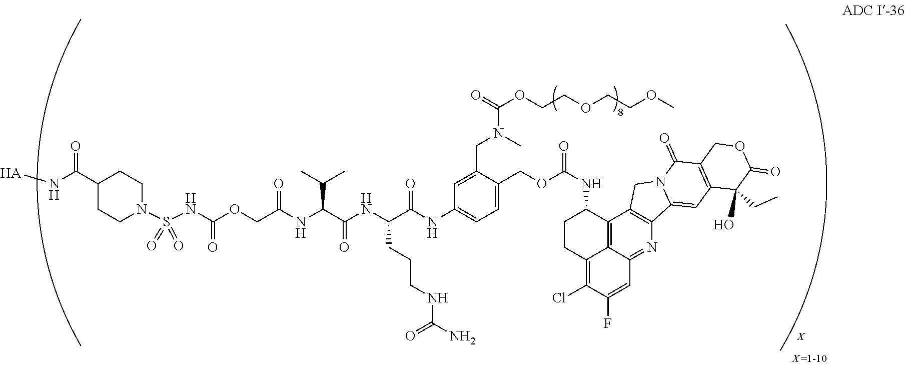 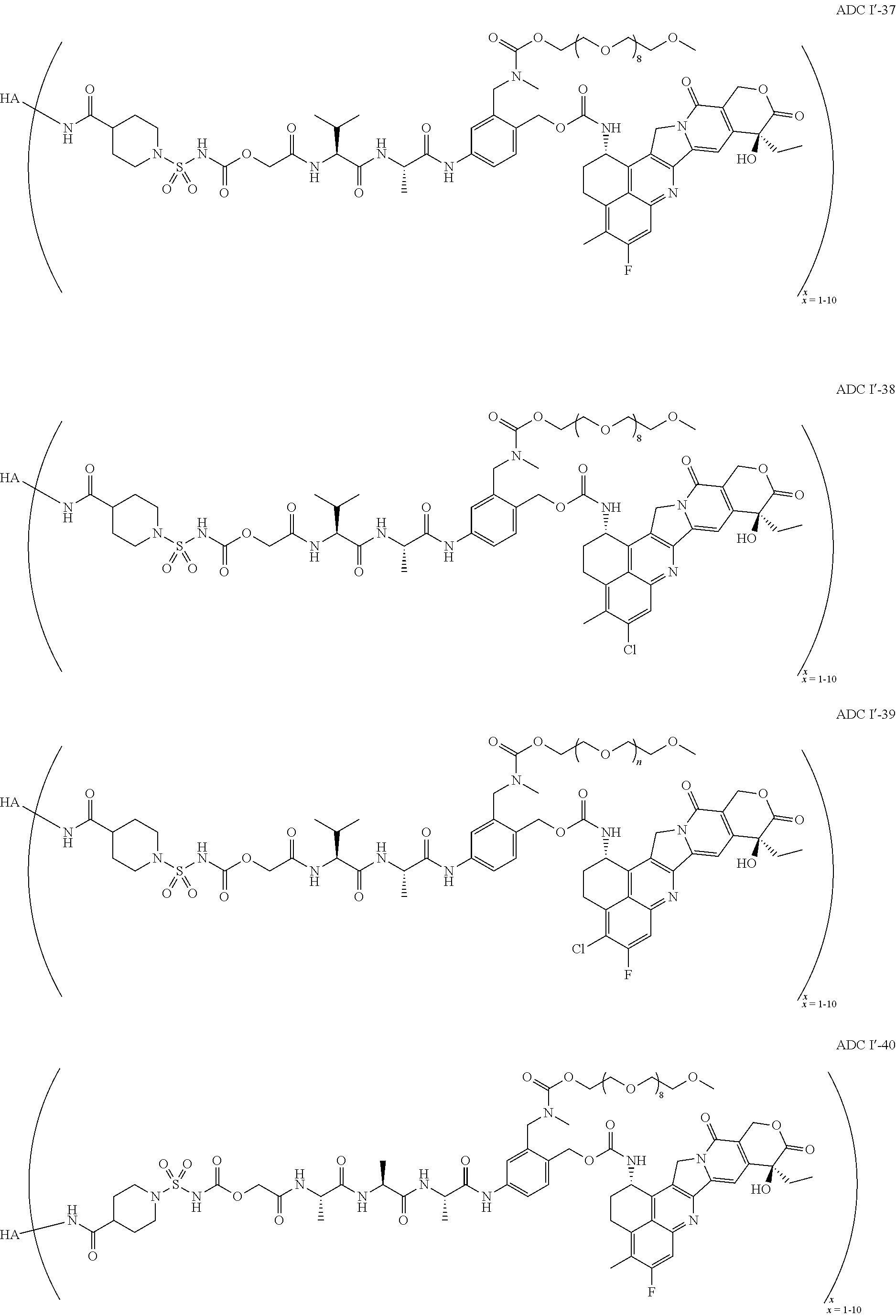 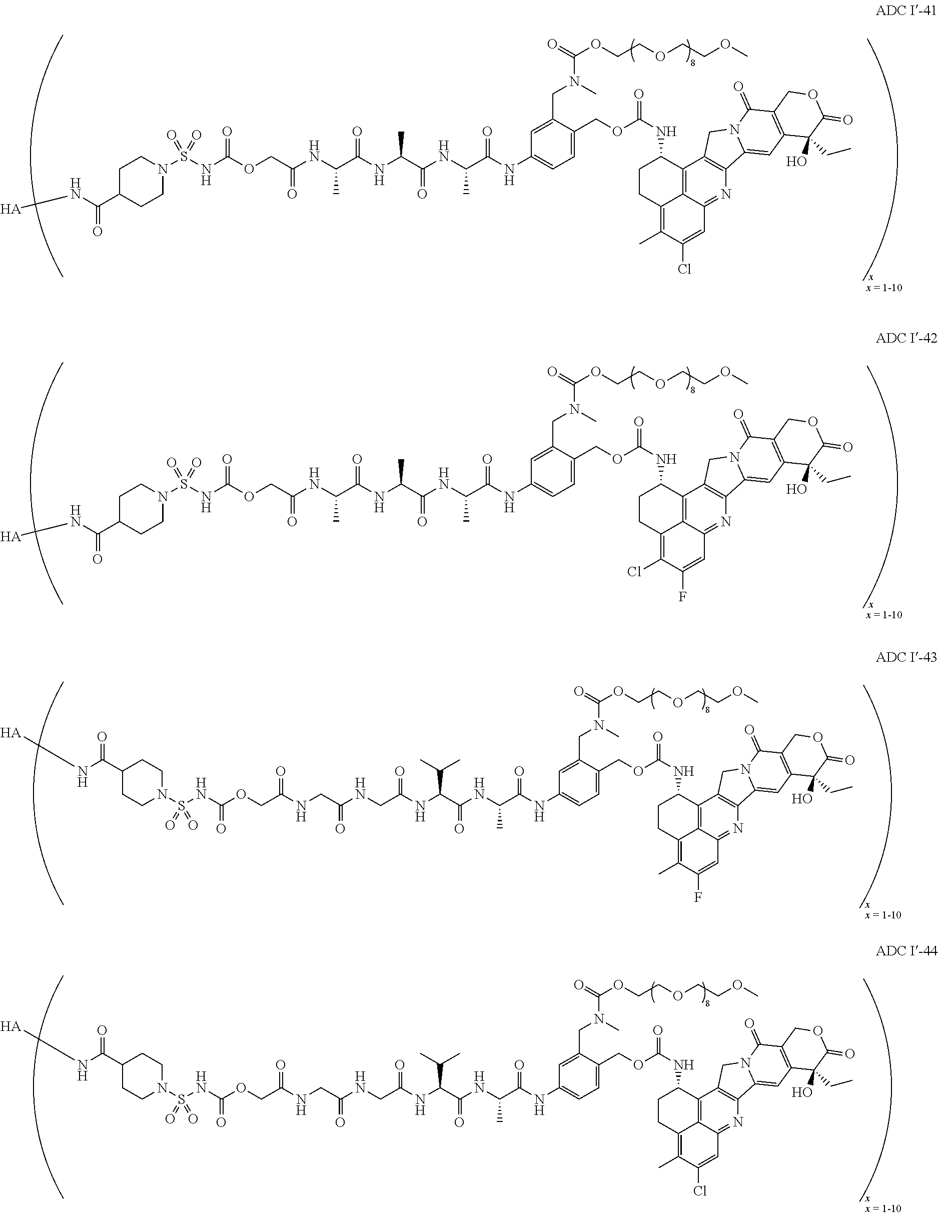 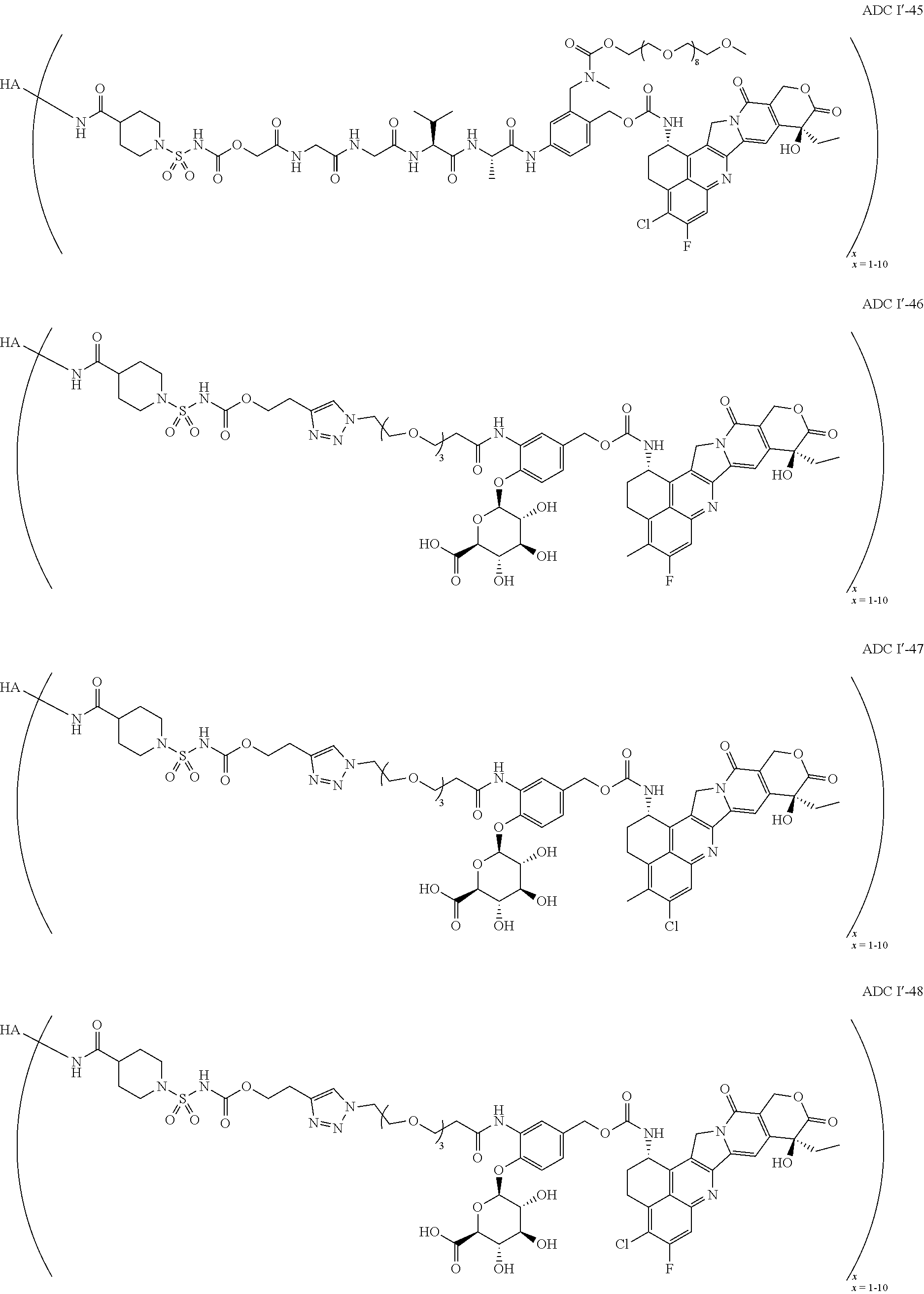 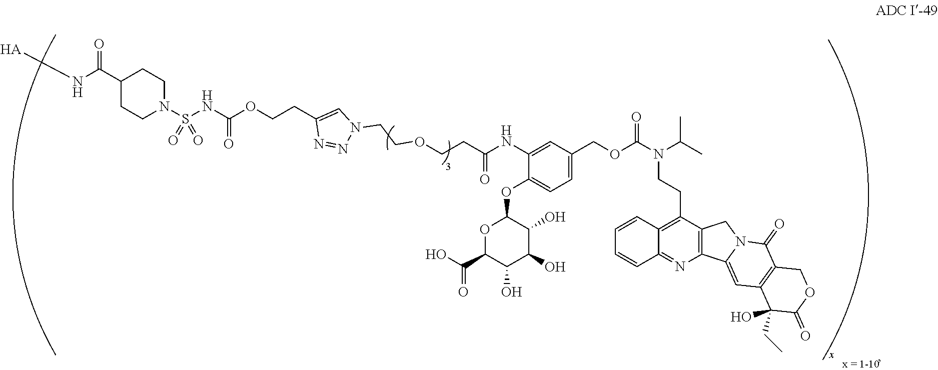
wherein, HA in each of the antibody-drug conjugate represents an antibody or antigen-binding fragment, preferably the antibody or antigen-binding fragment comprises:
(1) the following heavy chain variable region (VH) and/or light chain variable region (VL), wherein the CDRs are defined according to the Chothia numbering system;
(1a) a heavy chain variable region (VH) comprising the following 3 CDRs: a CDR-H1 having a sequence as set forth in SEQ ID NO: 5 or variant thereof, a CDR-H2 having a sequence as set forth in SEO ID NO: 6 or variant thereof, and a CDR-H3 having a sequence as set forth in SEO ID NO: 7 or variant thereof; and/or, a light chain variable region (VL) comprising the following 3 CDRs; a CDR-L1 having sequence as set forth in SEQ ID NO: 8 or variant thereof, a CDR-L2 having a sequence as set forth in SEQ ID NO: 9 or variant thereof, and a CDR-L3 having a sequence as set forth in SEO ID NO: 10 or variant thereof; or,
(1b) a heavy chain variable region (VH) comprising the following 3 CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO:22 or variant thereof, and/or, a light chain variable region (VL) comprising the following 3 CDRs: a CDR-L1 having a sequence as set forth in SEO ID NO: 23 or variant thereof a CDR-L2 having a sequence as set forth in SEQ ID NO:24 or variant thereof, a CDR-L3 having a sequence as set forth in SEO ID NO: 25 or variant thereof;
wherein, the variant described in any one of (1a) and (1b) has a sequence identity of at least 70%, at least 80%, at least 85%, at least 90%, at least 91%, at least 92% at least 93%, at least 94%, at least 95%, at least 96%, at least 97%, at least 98%, at least 99%, or 100% as compared with the sequence from which it is derived, or the variant has a substitution, deletion or addition of one or more am acids (e.g. a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following 3 CDRs; a CDR-H1 having a sequence as set forth in SEO ID NO: 18 or variant thereof, a CDR-H2 having a sequence as set forth in SEQ ID NO: 19 or variant thereof, and a CDR-H3 having a sequence as set forth in SEO ID NO: 7 or variant thereof, and/or, light chain variable region (VL) comprising the following 3 CDRs: a CDR-L1 having a sequence as set forth in SEQ ID NO: 8 or variant thereof a CDR-L2 having a sequence as set f SEQ ID NO: 9 or variant thereof and a CDR-L3 having a sequence as set forth in SEQ ID NO: 10 or variant thereof, or,
(2b) a heavy variable ion (VH) comprising the following 3 CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO. NO: 22 or variant thereof; and/or, a light chain variable region (VL) comprising the following 3 CDRs: a CDR-L1 having a sequence as set forth in SEQ ID NO: 23 or variant thereof, a CDR-L2 having a sequence as set forth in SEO ID NO: 24 or variant thereof, and a CDR-L3 having a sequence as set forth in SEO ID NO: 25 or variant thereof;
wherein the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition one or more amino acids (e.g., a substitution, deletion or addition 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following 3 CDRs: a CDR-H1 having a sequence as set forth in SEO ID NO: 11 or variant thereof, a CDR-H2 having a sequence as set forth in SEO ID NO: 12 or variant thereof, and a CDR-H3 having a sequence as set forth in SEQ ID NO: 7 or variant thereof; and/or, a light chain variable region (VL) comprising the following 3 CDRs: a CDR-L1 having a sequence as set forth in SEO ID NO: 8 or variant thereof a CDR-L2 having, sequence as set forth in SEQ ID NO: 9 or variant thereof, and a CDR-L3 having a sequence as set forth in SEO ID NO: 10 or variant thereof; or,
(3b) a heavy chain variable region (VH) comprising the following 3 CDRs: a CDR-H1 having a sequence as set forth in SEO ID NO: 26 or variant thereof, a CDR-H2 having a sequence as set forth in SEQ ID NO: 27 or variant thereof, and a CDR-H3 having a sequence as se or n SEQ ID NO. NO:: 22 (variant thereof; and/or, light chain variable region (VL) comprising the following 3 CDRs: a CDR-L1 having a sequence as set forth in SEQ ID NO: 23 or variant thereof, a CDR-L2 having a sequence as set forth in SEO ID NO: 24 or variant thereof, and a CDR-L3 having a sequence as set forth in SEQ ID NO: 25 or variant thereof;
wherein, the variant described in any one of (3a) and (3b) has a sequence identity of at least 70%, at least 80%, at least 85%, at least 90%, at least 91%, at least 92%, at least 93%, at least 94%, at least 95%, at least 96%, at least 97%, at least 98%, at least 99% or 100% as compared with the sequence from which it is derived, or the variant has a substitution, deletion or addition of one or more amino acids (e.g. a substitution, deletion or addition of 1, 2 or 3 amino acids) as compared with the sequence from which it is derived; preferably, the substitution is a conservative substitution;
or,
(4) the following heavy chain variable region (VL) and/or light chain variable region (VL) wherein the CDRs are defined according to the IMGT numbering system;
(4a) a heavy chain variable region (VH) comprising the following 3 CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof; and/or, a light chain variable region (VL) comprising the following 3 CDRs: a CDR-L1 having a sequence as set forth in SEQ ID NO: 16 or variant thereof, a CDR-L2 having a sequence as set forth in SEO ID NO: 17 or variant thereof, and a CDR-L3 having a sequence as set forth in SEQ ID NO: 10 or variant thereof; or,
(4b) a heavy chain variable region (VH) comprising the following 3 CDRs: a CDR-H1 having a sequence as set forth in SEQ ID NO: 28 or variant thereof, a CDR-H2 having a sequence as set forth in SEO ID NO: 29 or variant thereof and a CDR-H3 having a sequence as set forth in SEO ID NO. NO:: 30 or variant thereof, and/or, a light chain variable region (VL) comprising the following 3 CDRs: a CDR-L1 having a sequence as set forth in SEQ ID NO: 31 or variant thereof a CDR-L21 variance as set forth in SEQ ID NO: 32 or variant thereof, and a CDR-L3 having a sequence as set forth in SEO ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which derived, or the variant substitution, deletion or addition of one or more amino acids (e.g., a substitution, deletion or addition of 1, 2 or 3 amino acids) as compared with the sequence from which it is derived: preferably, the substitution is a conservative substitution;
preferably, the antibody or antigen-binding fragment thereof comprises:
(1) the following heavy chain variable region (VH) and light chain variable region (VL), wherein the CDRs are defined according to the Chothia numbering system:
(1a) a heavy chain variable region (VH) comprising the following 3 CDRs, a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or
(1b) a heavy chain variable region (VH) comprising the following 3 CDRs: a CDR-H1 having a sequence as set forth in SEQ ID NO: 20, CDR-H2 having a sequence as set forth in SEQ ID NO: 21, and a CDR-H3 having a sequence as set forth in SEQ ID NO: 22; and, a light chain variable region (VL) comprising the following 3 CDRs a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL) wherein the CDRs are defined according to the AbM umbering system:
(2a) a heavy chain variable region (VH) comprising the following 3 CDRs: a CDR-H1 having a sequence as set forth in SEQ ID NO: 18, a CDR-H2 having a sequence as set forth in SEO ID NO: 19, and a CDR-H3 having a sequence as set forth in SEQ ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or
(2b) a heavy chain variable region (VED) comprising the following 3 CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID: 22; and, a light chain variable region (VL) comprising the following 3 CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and light chain variable region (VL), wherein the CDRs are defined according to the Kabat numbering system:
(3a) a heavy chain variable region (VH) comprising the following 3 CDRs, a CDR-H1 having a sequence as set forth in SEQ ID NO: 11, a CDR-H2 having a sequence as set forth in SEQ ID NO: 12, and a CDR-H3 having a sequence as set forth in SEO ID NO: 7; and, a light chain variable region (VL) comprising the following 3 CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or
(3b) a heavy chain variable region (VH) comprising the following 3 CDRs: CDR-H1 having a sequence as set forth in SEQ ID NO: 26, a CDR-32 having a sequence as set forth in SEQ ID NO: 27, and a CDR-H3 having a sequence as set forth in SEQ ID NO: 22; and, a light chain variable region (VL) comprising the following 3 CDRs a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and light chain variable region (VL) wherein the CDRs are defined according to the IMGT numbering system,
(4a) a heavy chain variable region (VH) comprising the following 3 CDRs: a CDR-H1 having a sequence as set forth in SEQ ID NO: 13, a CDR-H2 having a sequence as set forth in SEO ID NO: 14, and a CDR-H3 having a sequence as set forth in SEO ID NO: 15; and, a light chain variable region (VL) comprising the following 3 CDRs: a CDR-L1 having a sequence as set forth in SEQ ID NO: 16, a CDR-12 having a sequence as set forth in SEQ ID NO: 17, and a CDR-L3 having a sequence as set forth in SEQ ID NO: 10; or
(4b) a heavy chain variable region (VED) comprising the following 3 CDRs: a CDR-H1 having a sequence as set forth in SEQ ID NO: 28, a CDR-H2 having sequence as set forth in SEQ ID NO: 29, as set forth in SEQ ID NO: 30; and, a light chain variable region (VL) comprising the following 3 CDRs: a CDR-L1 with a sequence as set forth in SEQ ID NO: 31, a CDR-L2 with a sequence as set forth in SEO ID NO: 32, and a CDR-L3 with a sequence as set forth in SEQ ID NO: 25;
n is an integer selected from 1 to 20, such as an integer of 1 to 12, 3 to 12, for example, n is 1, 2, 3, 4, 5, 6, 7, 8, 9, 10, 11, 12, 13, 14 or 15;
wherein, 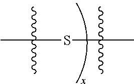 [SMILES: *S*]
represents a specific linkage between a sulfhydryl group in the antibody or antigen-binding fragment thereof and the linker; 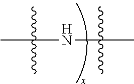 [SMILES: *N*]
represents a specific linkage between an amino group in the antibody or antigen-binding fragment thereof and the M fragment.

15. A composition, which comprises one or more antibody-drug conjugates according to claim 1, wherein the composition has a DAR value (drug-to-antibody conjugation ratio) of 1 to 10, for example: 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10, preferably 3 to 8, for example, 3.0 to 3.5, 3.0 to 4.0, 3.0 to 4.5, 3.0 to 5.0, 6.0 to 6.5, 6.0 to 7.0, 6.0 to 7.5, 6.0 to 8.0, 6.0 to 8.5, 6.5 to 7.0, 6.5 to 7.5, 6.5 to 8.0, 6.5 to 8.5, 7.0 to 7.5, 7.0 to 8.0 or 7.5 to 8.0; preferably 3.5 to 5.0 (e.g. 3.5, 3.6, 3.7, 3.8, 3.9, 4.0, 4.1, 4.2, 4.3, 4.4, 4.5, 4.6, 4.7, 4.8, 4.9, 5.0);
or, the DAR value of the composition is about 1.0 to 6.0, for example, about 1.0 to 5.5, about 1.0 to 5.0, about 1.5 to 6.0, about 1.5 to about 5.5, about 1.5 to 5.0, 2.0 to 5.5, about 2.0 to about 5.0, for example, about 1.0, about 1.01, about 1.02, about 1.03, about 1.04, about 1.05, about 1.06, about 1.07, about 1.08, about 1.09, about 1.1, about 1.11, about 1.12, about 1.13, about 1.14, about 1.15, about 1.16, about 1.17, about 1.18, about 1.19, about 1.2, about 1.21, about 1.22, about 1.23, about 1.24, about 1.25, about 1.26, about 1.27, about 1.28, about 1.29, about 1.3, about 1.31, about 1.32, about 1.33, about 1.34, about 1.35, about 1.36, about 1.37, about 1.38, about 1.39, about 1.4, about 1.41, about 1.42, about 1.43, about 1.44, about 1.45, about 1.46, about 1.47, about 1.48, about 1.49, about 1.5, about 1.51, about 1.52, about 1.53, about 1.54, about 1.55, about 1.56, about 1.57, about 1.58, about 1.59, about 1.6, about 1.61, about 1.62, about 1.63, about 1.64, about 1.65, about 1.66, about 1.67, about 1.68, about 1.69, about 1.7, about 1.71, about 1.72, about 1.73, about 1.74, about 1.75, about 1.76, about 1.77, about 1.78, about 1.79, about 1.8, about 1.81, about 1.82, about 1.83, about 1.84, about 1.85, about 1.86, about 1.87, about 1.88, about 1.89, about 1.9, about 1.91, about 1.92, about 1.93, about 1.94, about 1.95, about 1.96, about 1.97, about 1.98, about 1.99, about 2.0, about 2.01, about 2.02, about 2.03, about 2.04, about 2.05, about 2.06, about 2.07, about 2.08, about 2.09, about 2.1, about 2.11, about 2.12, about 2.13, about 2.14, about 2.15, about 2.16, about 2.17, about 2.18, about 2.19, about 2.2, about 2.21, about 2.22, about 2.23, about 2.24, about 2.25, about 2.26, about 2.27, about 2.28, about 2.29, about 2.3, about 2.31, about 2.32, about 2.33, about 2.34, about 2.35, about 2.36, about 2.37, about 2.38, about 2.39, about 2.4, about 2.41, about 2.42, about 2.43, about 2.44, about 2.45, about 2.46, about 2.47, about 2.48, about 2.49, about 2.5, about 2.51, about 2.52, about 2.53, about 2.54, about 2.55, about 2.56, about 2.57, about 2.58, about 2.59, about 2.6, about 2.61, about 2.62, about 2.63, about 2.64, about 2.65, about 2.66, about 2.67, about 2.68, about 2.69, about 2.7, about 2.71, about 2.72, about 2.73, about 2.74, about 2.75, about 2.76, about 2.77, about 2.78, about 2.79, about 2.8, about 2.81, about 2.82, about 2.83, about 2.84, about 2.85, about 2.86, about 2.87, about 2.88, about 2.89, about 2.9, about 2.91, about 2.92, about 2.93, about 2.94, about 2.95, about 2.96, about 2.97, about 2.98, about 2.99, about 3.0, about 3.01, about 3.02, about 3.03, about 3.04, about 3.05, about 3.06, about 3.07, about 3.08, about 3.09, about 3.1, about 3.11, about 3.12, about 3.13, about 3.14, about 3.15, about 3.16, about 3.17, about 3.18, about 3.19, about 3.2, about 3.21, about 3.22, about 3.23, about 3.24, about 3.25, about 3.26, about 3.27, about 3.28, about 3.29, about 3.3, about 3.31, about 3.32, about 3.33, about 3.34, about 3.35, about 3.36, about 3.37, about 3.38, about 3.39, about 3.4, about 3.41, about 3.42, about 3.43, about 3.44, about 3.45, about 3.46, about 3.47, about 3.48, about 3.49, about 3.5, about 3.51, about 3.52, about 3.53, about 3.54, about 3.55, about 3.56, about 3.57, about 3.58, about 3.59, about 3.6, about 3.61, about 3.62, about 3.63, about 3.64, about 3.65, about 3.66, about 3.67, about 3.68, about 3.69, about 3.7, about 3.71, about 3.72, about 3.73, about 3.74, about 3.75, about 3.76, about 3.77, about 3.78, about 3.79, about 3.8, about 3.81, about 3.82, about 3.83, about 3.84, about 3.85, about 3.86, about 3.87, about 3.88, about 3.89, about 3.9, about 3.91, about 3.92, about 3.93, about 3.94, about 3.95, about 3.96, about 3.97, about 3.98, about 3.99, about 4.0, about 4.01, about 4.02, about 4.03, about 4.04, about 4.05, about 4.06, about 4.07, about 4.08, about 4.09, about 4.1, about 4.11, about 4.12, about 4.13, about 4.14, about 4.15, about 4.16, about 4.17, about 4.18, about 4.19, about 4.2, about 4.21, about 4.22, about 4.23, about 4.24, about 4.25, about 4.26, about 4.27, about 4.28, about 4.29, about 4.3, about 4.31, about 4.32, about 4.33, about 4.34, about 4.35, about 4.36, about 4.37, about 4.38, about 4.39, about 4.4, about 4.41, about 4.42, about 4.43, about 4.44, about 4.45, about 4.46, about 4.47, about 4.48, about 4.49, about 4.5, about 4.51, about 4.52, about 4.53, about 4.54, about 4.55, about 4.56, about 4.57, about 4.58, about 4.59, about 4.6, about 4.61, about 4.62, about 4.63, about 4.64, about 4.65, about 4.66, about 4.67, about 4.68, about 4.69, about 4.7, about 4.71, about 4.72, about 4.73, about 4.74, about 4.75, about 4.76, about 4.77, about 4.78, about 4.79, about 4.8, about 4.81, about 4.82, about 4.83, about 4.84, about 4.85, about 4.86, about 4.87, about 4.88, about 4.89, about 4.9, about 4.91, about 4.92, about 4.93, about 4.94, about 4.95, about 4.96, about 4.97, about 4.98, about 4.99, about 5.0.

16. A pharmaceutical composition, which comprises the antibody-drug conjugate according to claim 1, and one or more pharmaceutical excipients.

17-20. (canceled)

21. A method for treating Her2-expressing cancer, the method comprising administering to an individual in need thereof a therapeutically effective amount of the antibody-drug conjugate according to claim 1;
preferably, wherein the cancer is selected from the group consisting of solid tumors or hematological malignancies, for example, gastric cancer, breast cancer, lung cancer (e.g., non-small cell lung cancer, specifically lung adenocarcinoma) or urothelial carcinoma.

22. (canceled)

23. A pharmaceutical composition, which comprises the composition according to claim 15, and one or more pharmaceutical excipients.

24. A method for treating Her2-expressing cancer, the method comprising administering to an individual in need thereof a therapeutically effective amount of the composition according to claim 15;
preferably, wherein the cancer is selected from the group consisting of solid tumors of hematological malignancies, for example, gastric cancer, breast cancer, lung cancer (e.g., non-small cell lung cancer, specifically lung adenocarcinoma) or urothelial carcinoma.

25. A method for treating Her2-expressing cancer, the method comprising administering to an individual in need thereof a therapeutically effective amount of the pharmaceutical composition according to claim 16;
preferably, wherein the cancer is selected from the group consisting of solid tumors or hematological malignancies, for example, gastric cancer, breast cancer, lung cancer (e.g. non-small cell lung cancer specifically lung adenocarcinoma) or urothelial carcinoma.

26. A method for treating Her2-expressing cancer, the method comprising administering to an individual in need thereof a therapeutically effective amount of the pharmaceutical composition according to claim 23;
preferably, wherein the cancer is selected from the group consisting of solid tumors or hematological malignancies, for example, gastric cancer, breast cancer, lung cancer (e.g., non-small cell lung cancer, specifically lung adenocarcinoma) or urothelial carcinoma.
